(19) United States
(12) Patent Application Publication
(10) Pub. No.: US 2024/0246992 A1
(43) Pub. Date: Jul. 25, 2024

(54) MACROCYCLIC LRRK2 KINASE INHIBITORS

(71) Applicants: LES LABORATOIRES SERVIER, SURESNES (FR); ONCODESIGN PRECISION MEDICINE (OPM), Dijon Cedex (FR)

(72) Inventors: Audrey DUMOULIN, Antony (FR); Petra Marcella BLOM, Destelbergen (BE); Alain DAUGAN, Vaugrigneuse (FR); Maxime LAUGEOIS, Paris (FR); Christopher Gaétan HOUSSEMAN, Massy (FR); Alexis DENIS, Paris (FR); Yann LAMOTTE, ANTONY (FR); Arnaud LE TIRAN, Croissy sur Seine (FR); Kenneth CHRISTENSEN, Copenhagen (DK)

(21) Appl. No.: 18/548,231

(22) PCT Filed: Mar. 17, 2022
(86) PCT No.: PCT/EP2022/056910

§ 371 Date: Aug. 29, 2023

(30) Foreign Application Priority Data
Mar. 18, 2021 (EP) 21305334.1
Sep. 24, 2021 (EP) 21306329.0

(51) Int. Cl. C07D 491/16; A61K 9/20; A61K 31/4162; A61K 31/4188; A61K 31/4192; A61K 31/429; A61K 31/437; A61K 31/496; A61K 31/4985; A61K 31/5025; A61K 31/519; A61K 31/5377; C07D 491/06; C07D 491/22; C07D 498/06; C07D 498/16

(52) U.S. Cl. CPC C07D 491/16; A61K 9/2009; A61K 9/2013; A61K 9/2018; A61K 9/2054; A61K 9/2059; A61K 31/4162; A61K 31/4188; A61K 31/4192; A61K 31/429; A61K 31/437; A61K 31/496; A61K 31/4985; A61K 31/5025; A61K 31/519; A61K 31/5377; C07D 491/06; C07D 491/22; C07D 498/06; C07D 498/16

(57) ABSTRACT

Compounds of formula (I) are useful as LRRK2 kinase inhibitors.

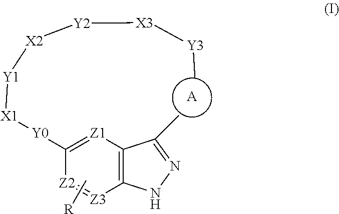

### FIELD OF THE INVENTION

The present invention relates to novel macrocyclic compounds and compositions containing said compounds acting as kinase inhibitors, in particular as inhibitors of LRRK2 (Leucine-Rich Repeat Kinase 2). Moreover, the present invention provides processes for the preparation of the disclosed compounds, pharmaceutical compositions containing them, as well as methods of using them, for instance as a medicine or diagnostic agent, in particular for the treatment and/or diagnosis of diseases impacted or modulated by LRRK2 kinase activity such as neurological disorders including Parkinson's disease and Alzheimer's disease, but also cardiac diseases or inflammatory disorders such as Crohn's disease.

### BACKGROUND OF THE INVENTION

Parkinson's disease is the most common movement disorder and the second most common neurodegenerative disease after Alzheimer's disease. Parkinson's disease affects approximately 1% of the population above 65 years and is characterized by the four classical core motor complications: resting tremor, bradykinesia, postural instability and muscular rigidity. Patients with Parkinson's disease are also impacted by a host of non-motor symptoms such as constipation, hyposmia, orthostatic hypotension, sleep disturbances including REM sleep behavior disorder, dementia, visual disturbances, depression, anxiety, hallucinations and mood swings.

Standard of care in Parkinson's disease is symptomatic relief of motor complications using dopamine replacement therapy such as the dopamine precursor L-dopa, dopamine agonists or compounds that impact the half-life of dopamine such as MAO-B inhibitors. As of today, there is no approved therapy to prevent, cure or delay the progression of Parkinson's disease.

The pathological hallmarks of Parkinson's disease are the loss of dopaminergic neurons in the substantia nigra pars compacta as well as postmortem evidence of protein inclusions, also known as Lewy bodies and Lewy neurites. In postmortem tissue from Parkinson's disease patients Lewy bodies and neurites are seen throughout the central nervous system and in peripheral tissues as well. A major component of the inclusions is the aggregated and misfolded α-synuclein protein phosphorylated at a serine at amino acid position 129 (Nature 388, 839-840, 1997; Nat Cell Biol 4, 160-64, 2002). Lewy bodies and neurites also contain proteins implicated in other neurodegenerative diseases such as the hyperphosphorylated tau protein which is a pathological hallmark of tauopathies such as Alzheimer's disease (AD), frontotemporal dementia (FTD), progressive supranuclear palsy (PSP) and corticobasal degeneration (CBD) (Biochem Soc Trans 26(3), 463-71, 1998; Am J Hum Genet 64(2), 414-21, 1999; J Neuropathol Exp Neurol 62(4), 389-97, 2003). The pathological process in Parkinson's disease is not restricted to the loss of dopaminergic neurons in the basal ganglia system. Distinct neuronal populations in other brain regions such as the neocortex, sleep nuclei or the raphe nucleus as well as peripheral organs and tissues such as the heart and the gastro-intestinal system are also impacted by degenerative processes in Parkinson's disease patients.

Leucine-rich repeat kinase 2 (LRRK2) is a 2527 amino acid protein with a molecular weight of 286 kDa that is encoded by the LRRK2 gene. It consists of the following functional and structural proteins domains: armadillo (ARM), ankyrin (ANK), leucine rich repeat (LRR), Ras of complex domain (Roc), c-terminal of Roc (COR), map kinase (MAPK) and tryptophan-aspartate repeat domain (WD40). LRRK2 exists primarily as a dimeric protein either associated with membrane structures or cytoplasmic localized. The armadillo, ankyrin, LRR and WD40 protein-protein interaction domains enables LRRK2 to interact with a host of different protein partners to impact its own as well as its partner proteins subcellular localization. The central enzymatic core of the LRRK2 protein containing the Roc-COR and the MAPK domain have distinct GTPase and ATPase enzymatic activities enabling LRRK2 to phosphorylate and control the function of intracellular substrates. LRRK2 impacts, via its enzymatic activity and substrate interactions, various subcellular processes and biological mechanisms important for trafficking of intracellular vesicular structures and organelles such as lysosomes, endosomes, autophagosomes, the Golgi and mitochondria. Structural work as well as modelling highlights how naturally occurring missense variation in functional and structural domains of LRRK2 impacts enzymatic activity (bioRxiv 2020.01.06.895367). In the inactive (open) LRRK2 conformation there are major interactions between the enzymatic GTPase (Roc-COR) and ATPase (MAPK) domains. In addition, the ultimate C-terminal proceeding the WD40 domain binds along the entire kinase (MAPK) domain. In the active (closed) LRRK2 conformation the LRR domain positions the autophosphorylation site Ser1292 in proximity to the kinase active site. Phosphorylation of LRRK2 at a cluster of serines immediately preceding the LRR domain enables the LRR domain of LRRK2 to bind to 14-3-3 proteins. Among those phosphorylation sites are serines (Ser) at the following amino acid positions: Ser910, Ser935, Ser955 and Ser973. Pathogenic LRRK2 mutations originating in the GTPase domain has diminished phosphorylation at these sites and therefore reduced 14-3-3 binding leading to increased microtubule network recruitment. All ATP-competitive LRRK2 inhibitors induce dephosphorylation at the Ser910, Ser935, Ser955 and Ser973 sites making these sites useful as surrogate target engagement markers (Biochem J 430(3), 405-13, 2010; J Neurochem 120(1), 37-45, 2012). The bona fide LRRK2 substrates consists of a subset of small Rab GTPases including Rab10 and Rab29. The Golgi-resident protein Rab29 also known as Rab7L1 is a Parkinson's disease susceptibility gene located at the PARK16 locus (Nat Genet 41(12), 1308-12, 2009).

Rare protein-encoding variants in the LRRK2 gene cause Parkinson's disease. The most common pathogenic variant causing autosomal dominant familial Parkinson's disease is the p.G2019S substitution which changes a glycine to a serine in the activation loop of the LRRK2 kinase domain rendering the p.G2019S variant more active than the wild type LRRK2 protein (Lancet 365(9457), 412-5, 2005). This results in increased autophosphorylation at the serine at amino acid position 1292 (Sci Transl Med, 4(164), 164ra161, 2012). The estimated worldwide prevalence of the p.G2019S mutation in patients with PD is 1-2%; whereas, in Ashkenazi Jewish and North African Arab-Berber populations the p.G2019S prevalence in PD patients is up to 30% and 40%, respectively (Lancet Neurol 7, 583-90, 2008; N Engl J Med 354(4), 424-5, 2006; Lancet Neurol 7, 591-4, 2008). The clinical manifestation of Parkinson's disease in patients carrying the p.G2019S mutation is indistinguishable from patients with the sporadic form of Parkinson's disease (Ann Neurol 57(5), 762-5, 2005). Besides p.G2019S seven additional rare LRRK2 exonic variants having non-synonymous amino acid substitutions in the central enzymatic core (p.N1437H; p.R1441C/G/H; p.Y1699C; p.S1761R; p.I2020T) also cause autosomal dominant Parkinson's disease (Parkinsonism Relat Disord 15(6), 466-7, 2009; Mov Disord 25(14), 2340-5, 2010; Neuron 44(4), 601-7, 2004; Parkinsonism Relat Disord 18(4), 332-8, 2012; Ann Neurol 57(6), 918-21, 2005; Mov Disord 27(1), 146-51, 2012). As with p.G2019S the clinical representations are indistinguishable from idiopathic PD (Neurology 70, 1456-60, 2008). LRRK2 missense variants exhibit increased Ser1292 phosphorylation, increased trans-Golgi recruitment by Rab29 and increased phosphorylation of Rab10 at amino acid position 73 (Rab10-Thr73) that can be reversed by LRRK2 inhibition (Sci Transl Med 4(164), 164ra161, 2012; EMBO J 37(1), 1-18, 2018; Proc Natl Acad Sci USA 111, 2626-31, 2014). Common protein-coding variants in the LRRK2 gene are also associated with risk of Parkinson's disease. Variants such as p.A419V, p.M1646T, p.R1628P and p.G2385R increase the risk of Parkinson's disease and have increased kinase activity (bioRxiv 447946, 2018) (Proc Natl Acad Sci USA 116(5), 1579-1584, 2019) whereas the p.N551K variant is associated with reduced risk of Parkinson's disease (Lancet Neurol 10(10), 898-908, 2011) and have reduced kinase activity (bioRxiv 447946, 2018). Evidence that LRRK2 also plays a role in sporadic Parkinson's disease comes from both genetic studies as well as postmortem analyses of PD brains. A single nucleotide polymorphism (SNP) at the LRRK2 genetic locus is genome-wide associated with risk of Parkinson's disease (Nat Genet 46(9), 989-93, 2014). This particular SNP variant is associated with increased LRRK2 expression (Sci Transl Med 9 (421), 2017) which is in agreement with the increased LRRK2 kinase activity observed in surviving dopamine neurons from postmortem brains of sporadic PD patients (Sci Transl Med 10 (451), 2018).

Thus, inhibitors of LRRK2 kinase activity can be used as therapies for both sporadic PD patients as well as for PD patients with LRRK2 mutations or Rab29/Rab7L1 polymorphisms.

Parkinson's disease risk loci containing several genes encoding proteins involved in endosomal-lysosomal processes such as GBA, SCARB2, GALC, VPS35, LAMP1, VPS13C, VPS35, TMEM175, ATP6V0A1 and CTSB have been identified by Genome Wide Association Study (GWAS) and linkage studies. LRRK2 also plays a key role in the endosomal-lysosomal system and in the processes linked to endosomal function such as autophagy and mitophagy.

LRRK2 interacts with the vacuolar H+-ATPase α subunit to regulate lysosomal pH and endosomal-lysosomal dysfunction induced by rotenone, a toxin known to be associated with increased risk of Parkinson's disease, can be alleviated by LRRK2 inhibition (Neurobiol Dis 134, 104626, 2020). Disease-causing LRRK2 mutations induce lysosomal stress by enlarging lysosomes (Hum Mol Genet 24(21), 6013-28, 2015). Likewise, an aspartate to asparagine missense mutation in the retromer complex protein VPS35 at amino acid position 620 (VPS35-D620N) causes late onset autosomal dominant familial Parkinson's disease. In the disease state the VPS35-D620N missense mutation disrupts trafficking of cathepsin D, the protease responsible for degradation of α-synuclein (Traffic 15(2), 230-44, 2014) and activates LRRK2 which leads to increased autophosphorylation at the LRRK2-Ser1292 site and increased Rab10-Thr73 phosphorylation (Biochem J 475(11), 1861-1883, 2018). In the lysosomes LRRK2 interacts with GBA that is causally linked with the lysosomal storage disorder Gaucher's disease and a risk gene for Parkinson's disease. LRRK2 missense mutations reduce GBA activity that can be counteracted by LRRK2 inhibition (Nat Commun 10(1), 5570, 2019). Reversely, GBA disease-relevant deficits in lysosomal biology processes in astrocytes can also be alleviated by LRRK2 inhibition (Mov Disord Feb. 8, 2020, doi: 10.1002 mds.27994). Missense mutations in the mitochondrial kinase PINK1 and the E3 ligase PARKIN both cause autosomal recessive early onset Parkinson's disease that is associated with mitochondrial dysfunction (Science 304(5674), 1158-60, 2004; Nature 392(6676), 605-8, 1998). LRRK2-dependent phosphorylation of Rab8a on threonine at amino acid position 72 is modulated by PINK1 phosphorylation of serine on amino acid position 111 on Rab8a (Biochem J. Mar. 30, 2020, doi: 10.1042/BCJ20190664). Besides this LRRK2 activity impairs mitophagy that under normal conditions is regulated by the PINK1/PARKIN pathway. This can be reversed by LRRK2 inhibition (Hum Mol Genet 28(10), 1645-1660, 2019). LRRK2 missense mutations cause mitochondrial DNA damage that can be reversed by gene corrections (Neurobiol Dis 62, 381-6, 2014) as well as with inhibitors of LRRK2 (Hum Mol Genet. 26(22), 4340-4351, 2017). This suggests that LRRK2 inhibitors are useful for treating lysosomal storage disorders such as Gaucher's disease, Krabbe's disease, Niemann-Pick's disease and Fabry's disease, disorders with mitochondrial deficits including early onset Parkinson's disease associated with PINK1 and PARKIN missense mutations as well as Parkinson's disease in patients with polymorphisms in genes encoding proteins involved in the endosomal-lysosomal system such as GBA, GALC, VPS35, VPS13C, ATP6V0A1, LAMP1, SCARB2, TMEM175 and CTSB.

Postmortem analysis of brains from Parkinson's disease patient carrying LRRK2 mutations show presence of α-synuclein pathology (JAMA Neurol. 72(1), 100-5, 2015). In preclinical Parkinson's disease (PD) models, p.G2019S aggravates PD-related pathology that can be reversed by LRRK2 inhibition. LRRK2 has been identified in Lewy bodies in nigral and brain stem regions (Neuropathol Appl Neurobiol 34(3), 272-83, 2008) and has also been shown to phosphorylate α-synuclein on Ser129 (Biochem Biophys Res Commun 387(1), 149-52, 2009). LRRK2 exonic variation is associated with risk of multiple system atrophy (Neurology 83(24), 2256-61, 2014) and LRRK2 missense mutations have also been reported in patients with multiple system atrophy (J Parkinsons Dis; 8(1), 93-100, 2018). Single nucleotide polymorphisms in the MAPT (tau) locus is associated with increased risk of Parkinson's disease and multiple system atrophy (Hum Genet 124(6), 593-605, 2009; Parkinsonism Relat Disord 30, 40-5, 2016). Tau pathology is also a prominent feature seen in Parkinson's disease patients with LRRK2 missense mutations (Acta Neuropathol Commun 7(1), 183, 2019). Overexpression of pathogenic LRRK2 in animal models increase tau pathology (Neurobiol Dis 40(3), 503-17, 2010). LRRK2 missense mutations have been reported in patients suffering from tauopathies such as progressive supranuclear palsy and corticobasal degeneration (Mov Disord. 32(1), 115-123, 2017). Common variation at the LRRK2 locus is associated with survival in the primary tauopathy progressive supranuclear palsy (bioRxiv 2020.02.04.932335) and GWAS studies have identified risk for frontotemporal dementia at the LRRK2 locus (PLoS Med 15(1), e1002487, 2018).

This suggests that LRRK2 inhibitors are useful for treating synucleinopathies and tauopathies including frontotemporal dementia, progressive supranuclear palsy, corticobasal degeneration and Alzheimer's disease.

LRRK2 mRNA and protein are broadly expressed but particular enriched in brain tissue as well as in peripheral organs more specifically kidney, lung, intestine and spleen. Besides this LRRK2 expression is highly enriched in immune cells in the brain and in neutrophils, B-cells, macrophages and monocytes in the periphery. LRRK2 mRNA and protein expression is induced after pro-inflammatory stimuli or pathogens thereby increasing LRRK2 kinase activity. In human peripheral blood mononuclear cells, the LRRK2 substrates Rab10 and Rab12 are phosphorylated after stimulation with reagents mimicking viral infections (Sci Rep 7(1), 10300, 2017). Consistent with LRRK2 biology playing a role in response to inflammatory stimuli LRRK2 missense mutations are associated with risk of the inflammatory bowel disorder Crohn's disease and GWAS studies has identified single nucleotide polymorphisms in the LRRK2 locus associated with genome wide significant risk of Crohn's disease (Inflamm Bowel Dis 17(12), 2407-15, 2011). In Ashkenazi Jewish populations there is a two- to four-fold increased prevalence of Crohn's disease and in the same population LRRK2 variants are associated with increased risk of Crohn's disease (PLoS Genet 14(5), e1007329, 2018). LRRK2 exonic variants such as p.N2081D and p.M2397T increase the risk of Crohn's disease and as observed for Parkinson's disease the protective haplotype variant p.N551K/p.R1348H lowers the risk of Crohn's disease. In cell-based studies the p.N2081D variant has increased kinase activity which leads to augmented Rab10 phosphorylation (bioRxiv 447946, 2018; Sci Transl Med 10(423), 2018). The biological link between Parkinson's disease and autoimmune disorders are further supported by studies finding that common genetic pathways which also includes LRRK2 are shared between Parkinson's disease and autoimmune disorders such as rheumatoid arthritis, ulcerative colitis and Crohn's disease (JAMA Neurol 74(7), 780-92, 2017). Consistent with this LRRK2 is also associated with risk of lupus (Oncotarget 8, 13754-61, 2017; J Transl Med 17(1), 37, 2019) and leprosy (N Engl J Med 361(27), 2609-18, 2009; PLoS One 8(8), e73103, 2013; PLoS Negl Trop Dis 10(2), e0004412, 2016).

Thus, LRRK2 inhibitors can be used for treatment of Crohn's disease and other autoimmune disorder such as but not restricted to rheumatoid arthritis, ulcerative colitis, lupus and leprosy. LRRK2 plays a role in tumor growth in renal and thyroid cancers by impacting MET signaling, and lowering of LRRK2 expression induces growth arrest (Proc Natl Acad Sci USA 108(4), 1439-44, 2011). LRRK2-PD patients have increased risks of leukemia as well as skin and colon cancers (Mov Disord 34(9), 1392-8, 2019). Carriers of p.G2019S also have an overall increased risk of non-skin cancer; in particular breast cancer and hormone-related cancers in females (JAMA Neurol 72(1), 58-65, 2015). Studies have shown that LRRK2 silencing promotes T-cell growth inhibition and facilitates apoptosis and cell cycle arrest (Int J Oncol 55(1), 21-34, 2019). LRRK2 is also differentially expressed in lung adeno- and lung squamous cell carcinomas as well as non-small-cell lung cancer (J Cell Physiol 234(7), 10918-25, 2019; J Cell Physiol 234(12), 22742-52, 2019).

Thus, LRRK2 inhibitors have anti-carcinogenic effects and can be used for treatment of skin cancer and non-skin cancers such as renal cancer, colon cancer, adeno- and squamous lung cancers, non-small-cell lung cancer, hormone-related cancer, thyroid cancer, leukemia and breast cancer.

Extended prior art is known in the field of LRRK2 inhibitors. The most recent patent applications filed in the field cover oligomeric derivatives such as compounds disclosed in WO2020/006267, non-macrocyclic or polycyclic structures such as compounds disclosed in WO2019/222173, WO2019/112269, WO2019/074809, WO2018/217946, WO2018/163066, WO2018/155916, WO2018/137618, WO2018/06931, and also macrocyclic derivatives such as compounds disclosed in WO2019/012093, WO2016/042089. Notwithstanding the huge amounts of structures elaborated over the last years, there is a continuing need to design new scaffolds having a better potency and selectivity to meet the unmet medical needs.

### DETAILED DESCRIPTION OF THE INVENTION

The present invention will be described below. In the following passages, different aspects of the invention are defined in more details. Each aspect so defined may be combined with any other aspect or aspects unless clearly indicated to the contrary. In particular, any feature indicated as being preferred or advantageous may be combined with any other feature or features indicated as being preferred or advantageous.

In a first aspect the present invention provides a compound of Formula (I)

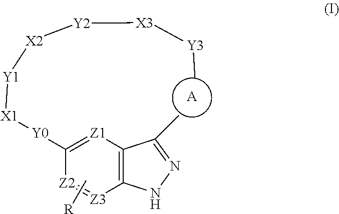

wherein:
R represents a hydrogen atom, a halogen atom or an alkyl group,
Z1, Z2, Z3, independently each represents a carbon or a nitrogen atom, it being understood that the 6-membered cycle containing Z1, Z2 and Z3 can have 0, 1 or 2 nitrogen atoms,
—X1- represents —O—, —S—, —S(O)—, S(O)₂— or —N(Ra)—, wherein Ra represents a hydrogen atom or an alkyl group,
—X2- represents —O—, —S—, —S(O), S(O)₂— or —N(Ra)—, wherein Ra represents a hydrogen atom or an alkyl group,
—X3- represents —O—, —S—, —S(O)—, S(O)₂—, —N(Ra)— or a bond, wherein Ra represents a hydrogen atom or an alkyl group,
—Y0- represents a bond or a linear or branched (C₁-C₃) alkanediyl group optionally substituted with one or more substituents, identical or different, selected from halogen atoms, polyhalogenoalkyl group, alkoxy group, hydroxy group, amino group, alkylamino group, dialkylamino group and cyano group,
—Y1- and —Y2-, identical or different, each represent a linear or branched (C₂-C₆) alkanediyl group, a linear or branched (C₂-C₆) alkenediyl group, or a linear or branched (C₃-C₆) cycloalkanediyl group, those groups being optionally substituted:
   with one or more halogen atoms,
   or with one substituent selected from polyhalogenoalkyl group, alkoxy group, hydroxy group, oxo group, amino group, alkylamino group, dialkylamino group and cyano group,
      it being understood that the carbon atom of the such defined —Y0-, —Y1- or —Y2- groups which is in alpha position of a heteroatom —X1-, —X2- or —X3-, cannot be substituted with an oxygen or a nitrogen heteroatom, unless it is an oxo group,
—Y3- represents a bond, a linear or branched (C₁-C₆) alkanediyl group, a linear or branched (C₂-C₆) alkenediyl group, or a linear or branched (C₃-C₆) cycloalkanediyl group, those groups being optionally substituted with one or more substituents, identical or different, selected from halogen atoms, polyhalogenoalkyl group, alkoxy group, hydroxy group, amino group, alkylamino group, dialkylamino group and cyano group,
   it being understood that the carbon atom of the such defined —Y3- group which is in alpha position of a heteroatom —X3- or A1 when A1 represents a nitrogen atom, cannot be substituted with an oxygen or a nitrogen heteroatom,
A represents
   an aromatic or partially hydrogenated cyclic group of the formula (a):

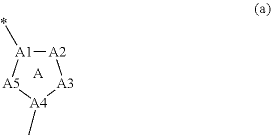

wherein
   A1, A4 each independently represents a carbon atom or a nitrogen atom,
   A2, A3, A5 each independently represents a carbon atom, an oxygen atom, a sulfur atom or a nitrogen atom,
      it being understood that when A1 represents a nitrogen atom, it cannot be linked to a heteroatom of the —Y0-X1-Y1-X2-Y2-X3-Y3- chain,
      it being understood that * means that the bond is linked to Y3,
   or an aromatic or partially hydrogenated cyclic group of the formula (b):

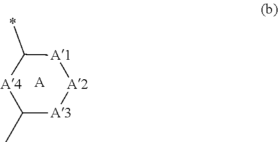

wherein
A′1, A′2, A′3, A′4 each independently represents a carbon atom or a nitrogen atom,
   it being understood that * means that the bond is linked to Y3,
      the cyclic group A such defined being optionally substituted with one or more substituents, identical or different, selected from halogen atoms, alkyl group, alkoxy group, hydroxy group, oxo group, alkoxyalkyl group, alkoxyalkoxy group, polyhalogenoalkyl group, polyhalogenoalkoxy group, heterocycloalkyl group, heterocycloalkylalkyl group, (alkoxyalkyl)(alkyl)amino group, amino group, alkylamino group, dialkylamino group, cycloalkyl group, (heterocycloalkyl)(alkyl)amino group, dialkylaminoalkyl group, heterocycloalkylalkoxy group, cyano group and cyanoalkyl group,
      wherein the heterocycloalkyl and cycloalkyl group such defined can be optionally substituted by one or more substituents chosen from alkyl group, halogen atoms, polyhalogenoalkyl group, polyhalogenoalkoxy group, alkoxy group, alkoxyalkyl group, hydroxy group, cyano group and oxo group,
      their enantiomers, diastereoisomers, tautomers, racemic, hydrate, solvates, N-oxide, isotopes, deuterated derivatives and addition salts thereof with a pharmaceutically acceptable acid or base.

When describing the compounds of the invention, the terms used are to be construed in accordance with the following definitions, unless a context dictates otherwise:

The term “alkyl” by itself or as part of another substituent refers to fully saturated monovalent hydrocarbon radical, including corresponding deuterated derivatives. Alkyl groups of this invention have from 1 to 6 carbon atoms (also represented by (C₁-C₆)), unless specified otherwise. Alkyl groups may be linear or branched and may be optionally substituted as indicated herein. Examples of alkyl groups are methyl, ethyl, n-propyl, i-propyl, butyl and its isomers (e.g. n-butyl, i-butyl and t-butyl), pentyl and its isomers, hexyl and its isomers.

The term “alkanediyl” means a fully saturated divalent hydrocarbon radical having two single bonds for attachment to two other groups, and can be represented as “-(alkyl)-” group wherein alkyl is as defined above. Alkanediyl groups of this invention can have from 1 to 6 carbon atoms (also represented by (C₁-C₆)), unless specified otherwise (for example (C₁-C₃) meaning from 1 to 3 carbon atoms, (C₂-C₆) meaning from 2 to 6 carbon atoms . . . ), may be linear or branched, may include spiranic structure, and may be substituted as indicated herein. Non-limiting examples of alkanediyl groups includes: —CH₂—, —CH₂—CH₂—, —CD₂-, —CD₂-CD₂-, —CH(CH₃)—, —CH(CH₂—CH₃)—, —CH(i-Pr)—, —C(CH₃)(CH₃)—, —CH₂—C(CH₃)(CH₃)—, —CH₂—CH₂—C(CH₃)(CH₃)—,

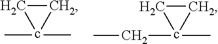

—CH₂—CH(i-Pr)—, —CH(i-Pr)—CH₂—, —CH₂—CH(i-Bu)-, —CH(i-Bu)-CH₂—, —CH(CH₃)—CH₂—, —CH₂—CH(CH₃)—, —CH₂—CH₂—CH₂—, —CD₂-CD₂-CD₂-, —CH(CH₃)—CH₂—CH₂—, —CH₂—CH₂—CH(CH₃)—, —CH₂—CH(CH₃)—CH₂—, —CH(CH₃)—CH₂—CH(CH₃)—, —CH₂—CH₂—CH(CH₂—CH₃)—, —CH(CH₂—CH₃)—CH₂—CH₂—, —CH(CH₂—CH₃)—CH₂—CH(CH₃)—, —CH(CH₃)—CH₂—CH(CH₂—CH₃)—, it being possible for those groups, when indicated, to be further substituted. For example, an alkanediyl group substituted by an alkoxy group will include, but will not be limited to, —CH(OCH₃)—, —CH(OCH₃)—CH(CH₃)—, —CH₂—CH₂—CH(OCH₃)—, —CH(OCH₃)—CH₂—CH₂—, —CH₂—CH₂—CH(CH₂—OCH₃)—, —CH(CH₂—OCH₃)—CH₂—CH₂—, —CH(O—CH₂—CH₃)—CH₂—, —CH₂—CH(O—CH₂—CH₃)—. As nonlimited other example, an alkanediyl group substituted by a cycloalkyl group will include —CH₂—CH(cy-Pr)—, —CH(cy-Pr)—CH₂—, wherein cy-Pr means cyclopropyl. An alkanediyl group substituted by an oxo group will include for example, but will not be limited to —C(O)—CH₂—, —CH₂—C(O)—, —C(O)—C(CH₃)₂—, —C(CH₃)₂—C(O)—, —C(O)—CH(CH₃)—, —CH(CH₃)—C(O)—, —C(O)—(CH₂)₂—, —(CH₂)₂—C(O)—, —C(O)—CH₂—CH(CH₃)—, —C(O)—CH(CH₃)—CH₂—, —CH₂—CH(CH₃)—C(O)—, —CH(CH₃)—CH₂—C(O)—,

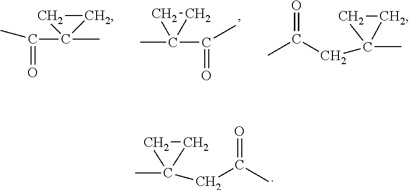

An alkanediyl group substituted by one or more halogen atoms includes for example, but will not be limited to —CHF—, —CHF—CH₂—, —CF₂—, —CF₂—CH₂—, —CH₂—CF₂—. An alkanediyl group substituted by a heterocycloalkyl group will include for example, but will not be limited to, —CH₂—CH(tetrahydropyranyl)-, —CH(tetrahydropyranyl)-CH₂—, —CH₂—CH(oxolanyl)-, —CH(oxolanyl)-CH₂—.

The term “alkenediyl” means an alkanediyl as described above containing one or more double bond. Alkenediyl groups of this invention comprise from 2 to 6 carbon atoms, may be linear or branched, and may be substituted as indicated herein. Non-limiting examples of alkenediyl groups includes: —CH₂—CH═CH—CH₂—, —CH(Me)-CH═CH—CH₂—.

The term “cycloalkanediyl” means an alkanediyl as described above wherein two consecutive carbon atoms of the chain can be part of a cyclic hydrocarbon structure. Cycloalkanediyl groups of this invention comprise from 3 to 6 carbon atoms, may be linear or branched, and may be substituted as indicated herein. Non-limiting examples of cycloalkanediyl groups includes:

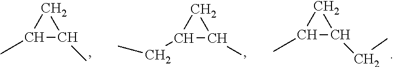

The term “cycloalkyl” by itself or as part of another substituent is a monovalent, saturated, or unsaturated hydrocarbon group having one or two cyclic structures, including corresponding deuterated derivatives. Cycloalkyl includes all saturated, partially saturated or aromatic hydrocarbon groups having one or two cyclic structures. Cycloalkyl groups comprise 3 or more carbon atoms and generally, according to this invention comprise from 3 to 10 carbon atoms.

Examples of cycloalkyl groups having one cyclic structure include but are not limited to phenyl, cyclopropyl, cyclobutyl, cyclopentyl, cyclohexyl.

When a bi-cyclic ring structure is envisaged, the two rings can be:
fused, meaning they share a common bond; exemplary cycloalkyl bi-cyclic fused systems include but is not limited to naphthalenyl, bicyclo[1.1.0]butanyl, octahydropentalenyl, decahydronaphthalenyl, octahydro-1H-indenyl;
linked via a bond between the two cyclic structures; exemplary cycloalkyl bi-cyclic linked systems include but is not limited to bi-phenyl, bi-cyclopropanyl, bi-cyclopentenyl, bi-cyclohexanyl, cyclopropylcyclohexanyl, cyclopropylcyclopentanyl;
bridged meaning that the two rings share three or more atoms, separating the two bridgehead atoms by a bridge containing at least one atom; exemplary cycloalkyl bi-cyclic bridged systems include but is not limited to bicyclo[2.2.1]heptanyl, bicyclo[2.2.2]octanyl;
or represent a spiro bi-cyclic ring system wherein the two rings are connected through a single atom; exemplary cycloalkyl spiro bi-cyclic systems include but is not limited to spiro[2.2]pentanyl, spiro[2.4]heptanyl, spiro[4.4]nonanyl, spiro[5.5]undecanyl.

The “cycloalkyl group” such defined can be optionally substituted by 1 to 3 substituents chosen from alkyl group, halogen atoms, polyhalogenoalkyl group, polyhalogenoalkoxy group, alkoxy group, alkoxyalkyl group, hydroxy group, cyano group and oxo group.

The term “alkoxy” by itself or as part of another substituent refers to an (alkyl)-O— group wherein “alkyl” is as defined above. Non-limiting examples of alkoxy groups includes methoxy, ethyloxy, n-propyloxy, i-propyloxy, butyloxy (and its isomers), pentyloxy (and its isomers), hexyloxy (and its isomers).

The term “alkoxyalkyl” refers to an (alkyl)-O-(alkyl)- group wherein “alkyl” is as defined above. Non-limiting examples include CH₃—O—CH₂—, CH₃—O—CH₂—CH₂—.

The term “alkoxyalkoxy” refers to an (alkyl)-O-(alkyl)-O— group wherein “alkyl” is as defined above. Non-limiting examples include CH₃—O—CH₂—CH₂—O—.

The term “alkylamino” refers to an —NH-(alkyl) group wherein “alkyl” is as defined above. Non-limiting examples include —NH—CH₃, —NH—CH₂—CH₃, —NH—CH(CH₃)₂.

The term “dialkylamino” refers to an —N(alkyl)(alkyl) group wherein “alkyl” is as defined above. Non-limiting examples include —N(CH₃)₂, —N(CH₃)(CH₂—CH₃).

The term “polyhalogenoalkyl” refers to an alkyl group as defined above wherein one or more hydrogen atom, carried by the same or different carbon atoms, is replaced by one or more halogen atoms. Non-limiting examples include fluoromethyl, difluoromethyl, trifluoromethyl, 2-chloroethyl.

The term “polyhalogenoalkoxy” refers to a (polyhalogenoalkyl)-O— group wherein “polyhalogenoalkyl” is as defined above. Non-limiting examples includes fluoromethoxy, difluoromethoxy, trifluoromethoxy, 2-chloroethoxy.

The term “heterocycloalkyl” means a monovalent mono- or bi-cyclic aromatic or non-aromatic carbocyclic group containing from 3 to 10 ring members and containing from 1 to 3 heteroatoms selected from oxygen atom, sulfur atom and nitrogen atom. The heterocycloalkyl group can be linked by a carbon or a nitrogen atom when possible. The heterocycloalkyl group such defined can be a monocyclic ring system or a bi-cyclic ring system. Heterocycloalkyl monocyclic ring system include but is not limited to pyridinyl, piperazinyl, piperidinyl, tetrahydropyridinyl, tetrahydropyranyl, pyrrolidinyl, dihydropyrrolyl, oxolanyl, dihydrofuranyl, morpholinyl, pyrazolyl, azetidinyl, oxetanyl, triazolyl. When a bi-cyclic ring system is envisaged, the two rings can be:
fused, meaning they share a common bond; exemplary heterocycloalkyl bi-cyclic fused systems include but is not limited to indolyl, indolinyl, benzopyranyl, benzofuranyl, naphthyridinyl, quinolinyl, pyridopyrazinyl, pyridopyridazinyl, pyridopyrimidinyl, dihydroquinolinyl, tetrahydroquinolinyl, dihydrobenzofuranyl, benzopyranyl, dihydrobenzopyranyl;
linked via a bond between the two cyclic structures; exemplary heterocycloalkyl bi-cyclic linked systems include but is not limited to phenylpyridinyl, bipyridinyl, oxetanylpyridinyl, oxetanylpiperidinyl, oxetanyltetrahydropyridinyl, pyrrolidinylpiperidinyl, pyrrolidinyltetrahydropyridinyl, pyrrolidinylpyridinyl, oxetanylpiperazinyl, pyrrolidinylpiperazinyl;
bridged meaning that the two rings share three or more atoms, separating the two bridgehead atoms by a bridge containing at least one atom; exemplary heterocycloalkyl bi-cyclic bridged systems include but is not limited to azabicyclo[2.2.1]heptanyl, oxaazabicyclo[2.2.1]heptanyl;
or represent a spiro bi-cyclic ring system wherein the two rings are connected through a single atom; exemplary heterocycloalkyl spiro bi-cyclic systems include but is not limited to oxaspirooctane, azaspirooctane, diazaspirooctane, oxaazaspirooctane, oxaspirononane, azaspirononane, diazaspirononane, oxaazaspirononane.

The “heterocycloalkyl group” such defined can be optionally substituted by 1 to 3 substituents chosen from alkyl group, halogen atoms, polyhalogenoalkyl group, polyhalogenoalkoxy group, alkoxy group, alkoxyalkyl group, hydroxy group, cyano group and oxo group.

The term “heterocycloalkylalkyl” refers to a (heterocycloalkyl)-(alkyl)- group wherein the heterocycloalkyl and the alkyl moieties are as defined above. Non-limiting examples include morpholinylmethyl, pyrrolidinylmethyl, piperazinylmethyl, piperidinylmethyl.

The term “halogen atoms” means a fluorine, chlorine, bromine or iodine atom.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid etc. . . . .

Among the pharmaceutically acceptable bases there may be mentioned, without implying any limitation, sodium hydroxide, potassium hydroxide, triethylamine, tert-butylamine etc. . . .

Specific embodiments and most preferred features of compounds of formula (I) of the invention are described below. Characteristics of those specific embodiments and features can be taken alone or combined to generate new specific embodiments.

In a specific embodiment, the invention more preferably refers to compounds of formula (I) wherein R represents a hydrogen atom.

In another embodiment, R represents advantageously a halogen atom, and most preferably a fluorine or a chlorine atom. Alternatively R is an alkyl group, and most preferably a methyl group.

R is preferably linked to Z2 or Z3 when Z2 or Z3 represents a carbon atom.

In another specific preferred embodiment of the invention, Z1, Z2 and Z3 represent simultaneously a carbon atom.

In an advantageous alternative embodiment, one of Z1, Z2 or Z3 is a nitrogen atom while the two others represent a carbon atom. More particularly when one of Z1, Z2 or Z3 represents a nitrogen atom, it is preferentially Z1 or Z2.

Another specific embodiment of the invention relates to compounds of formula (I) wherein —X1- represents —O— or —NH—. More preferably, —X1- represents —O—.

In another specific embodiment of the invention, —X2- advantageously represents —O—.

Alternatively, —X2- represents —NH— or —N(Me)-.

Another specific embodiment of the invention relates to compounds of formula (I) wherein preferred values for —X3- are —O— or a bond. More advantageously —X3- is a bond.

Another specific embodiment of the invention relates to compounds of formula (I) wherein —Y0- represents a bond.

In another specific embodiment of the invention, —Y1- represents an alkanediyl group linear or branched having 2, 3, 4 or 5 carbon atoms. —Y1- is preferably not substituted or substituted with one or more halogen atom. Advantageously —Y1- represents —(CH₂)₂—, —CF₂—CH₂—, —CH(CH₃)—CH₂—, —CH₂—CH(CH₃)—, —CH(CH₃)—CH(CH₃)—, —(CH₂)₃—, —(CH₂)₄—, —CH(CH₃)—(CH₂)₂—, —(CH₂)₂—CH(CH₃)—, —CH₂—CF₂—CH₂—, —CH₂—CHF—CH₂— or —CH(CH₃)—CH₂—CH(CH₃)—.

In another specific embodiment of the invention, —Y2- represents an alkanediyl group linear or branched having 2, 3, 4 or 5 carbon atoms. —Y2- is preferably not substituted. Advantageously —Y2- represents —(CH₂)₂—, —(CD₂)₂-, —(CH₂)₃—, —(CD₂)₃-, —CH(CH₃)—CH₂—, —CH₂—CH(CH₃)—, —CH₂—CH(CH₃)—CH₂—, —CH(CH₃)—(CH₂)₂—, —(CH₂)₂—CH(CH₃)—, —CH₂—CF₂—CH₂— or —(CH₂)₄—. An advantageous alternative for —Y2- is *—C(O)—(CH₂)₂—, *—C(O)—CH₂—CH(CH₃)—, *—C(O)—CH(CH₃)— or *—C(O)—CH₂—, wherein * means the link to —X2-.

Another specific embodiment of the invention relates to compounds of formula (I) wherein —Y3- represents a bond. Alternatively, —Y3- represents an alkanediyl group linear or branched having 1, 2 or 3 carbon atoms. Advantageously —Y3- represents —CH₂—, —(CH₂)₂—, —(CD₂)₂-, —CH(CH₃)—, —CH₂—CH(CH₃)—, —CH₂—CHF—, —CHF—CH₂— or —CH(CH₃)—CH₂—.

In another specific embodiment —Y1- and —Y2-, identical or different, each represents an unsubstituted alkanediyl group linear or branched having 3 or 4 carbon atoms, and —Y3- represents a bond.

Another specific embodiment of the present invention is represented by compounds of formula (I) for which A represents a group of formula (b):

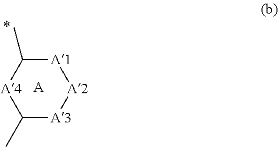

Preferred values for (A′ 1, A′2, A′3, A′4) are:
four carbon atoms, or
three carbon atoms and one nitrogen atom, more preferably the nitrogen atom being in A′4 or A′2,
or two carbon atoms and two nitrogen atoms.

A′3 is advantageously a carbon atom.

As a particular embodiment of the invention, A represents the following preferred scaffolds, being represented herein without any substitution:

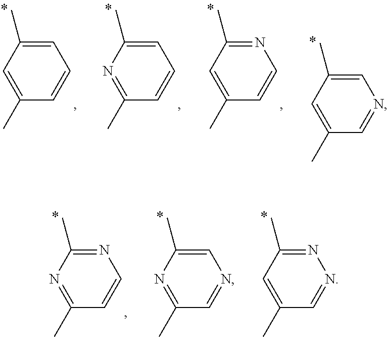

Most preferred embodiment for A is phenyl or pyridinyl group. An advantageous alternative for A is pyrazinyl group.

Another specific embodiment for A is represented by a group of formula (a):

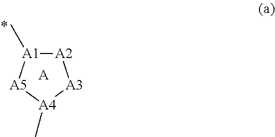

Most preferred scaffold of formula (a) contains one, two or three heteroatoms, one of them being a nitrogen atom. Representative scaffolds of formula (a) are as follows, being represented herein without any substitution:

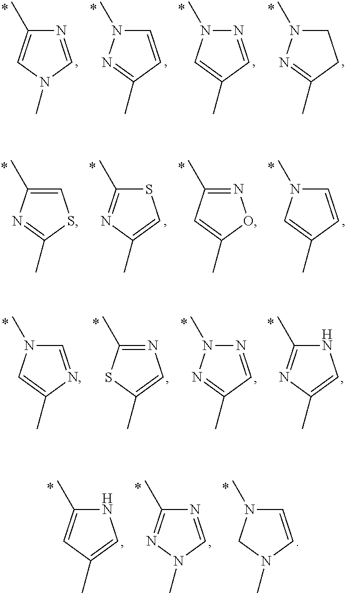

Advantageously, A represents a triazolyl or a pyrazolyl groups.

Preferentially the group A of the compounds of formula (I) is not substituted.

When the group A of the compounds of formula (I) is substituted, most preferred substitutions include halogen atoms, cyano group, cyanoalkyl group, alkoxy group, alkyl group, oxo group, cycloalkyl group and heterocycloalkyl group. Particularly, cycloalkyl group and heterocycloalkyl group are not substituted, or substituted with halogen atoms, alkoxy group, hydroxy group, or heterocycloalkyl group as preferred optional substituents.

Most preferred heterocycloalkyl group include pyrrolidinyl group, piperazinyl group, morpholinyl group, azetidinyl group, piperidinyl group, tetrahydropyridinyl group, tetrahydrofuranyl group, dihydrofuranyl group, oxetanyl group, pyrazolidinyl group.

Most preferred substitutions of the group A are fluorine or bromine atom, methoxy group, methyl group, ethyl group, cyanomethyl group, —C(CH₃)₂—CN group, pyrrolidinyl group unsubstituted or substituted, piperazinyl group unsubstituted or substituted, azetidinyl group unsubstituted or substituted, and morpholinyl group.

Another specific embodiment of the invention is represented by compounds of formula (I-a):

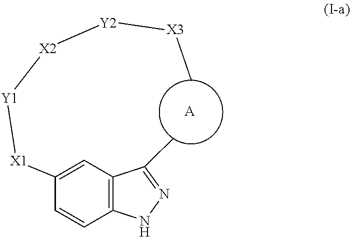

wherein X1, X2, X3, Y1, Y2 and A are as defined for formula (I).

In another preferred embodiment the invention concerns compounds of formula (I-b):

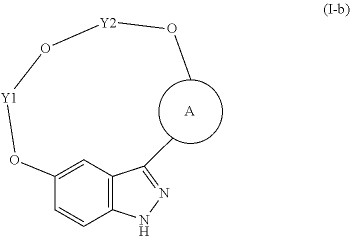

wherein Y1, Y2 and A are as defined for formula (I).

In another preferred embodiment the invention concerns compounds of formula (I-ba):

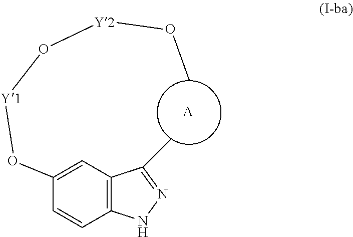

wherein A is as defined for formula (I), and Y′1 and Y′2, identical or different, each represents a linear or branched (C₂-C₆) alkanediyl group unsubstituted or substituted with one or more groups selected from halogen atoms and alkoxy group.

In another preferred embodiment the invention concerns compounds of formula (I-b1):

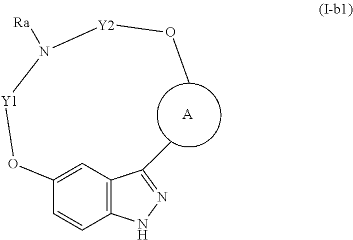

wherein Y1, Y2, Ra and A are as defined for formula (I).

In another preferred embodiment the invention concerns compounds of formula (I-b1a):

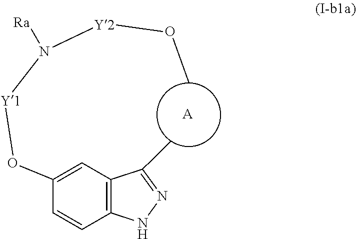

wherein A and Ra are as defined for formula (I), and Y′1 and Y′2, identical or different, each represents a linear or branched (C₂-C₆) alkanediyl group unsubstituted or substituted with one or more groups selected from halogen atoms and alkoxy group.

In another preferred embodiment the invention concerns compounds of formula (I-b1b):

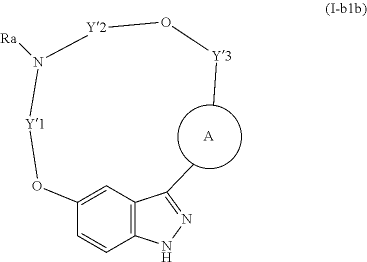

wherein A and Ra are as defined for formula (I), Y′1 and Y′2, identical or different, each represents a linear or branched (C₂-C₆) alkanediyl group unsubstituted or substituted with one or more groups selected from halogen atoms and alkoxy group, and Y′3 represents a linear or branched (C₁-C₆) alkanediyl group unsubstituted or substituted with one or more groups selected from halogen atoms and alkoxy group.

In another preferred embodiment the invention concerns compounds of formula (I-b1c):

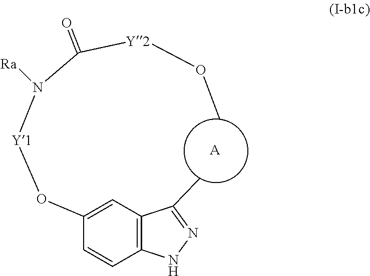

wherein A and Ra are as defined for formula (I), Y′1 represents a linear or branched (C₂-C₆) alkanediyl group unsubstituted or substituted with one or more groups selected from halogen atoms and alkoxy group, and Y″2 represents a linear or branched (C₁-C₅) alkanediyl group unsubstituted or substituted with one or more groups selected from halogen atoms and alkoxy group.

In another preferred embodiment the invention concerns compounds of formula (I-b1d):

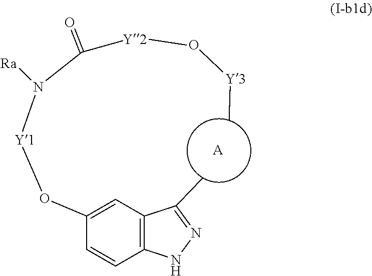

wherein A and Ra are as defined for formula (I), Y′1 represents a linear or branched (C₂-C₆) alkanediyl group unsubstituted or substituted with one or more groups selected from halogen atoms and alkoxy group, Y″2 represents a linear or branched (C₁-C₅) alkanediyl group unsubstituted or substituted with one or more groups selected from halogen atoms and alkoxy group, and Y′3 represents a linear or branched (C₁-C₆) alkanediyl group unsubstituted or substituted with one or more groups selected from halogen atoms and alkoxy group.

Another preferred specific embodiment of the invention concerns compounds of formula (I-c):

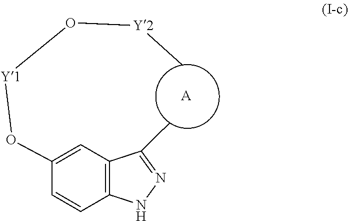

wherein Y1, Y2 and A are as defined for formula (I).

Another preferred specific embodiment of the invention concerns compounds of formula (I-ca):

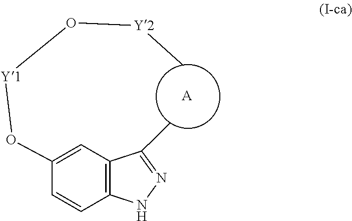

wherein A is as defined for formula (I), and Y′1 and Y′2, identical or different, each represents a linear or branched (C₂-C₆) alkanediyl group unsubstituted or substituted with one or more groups selected from halogen atoms and alkoxy group.

Another preferred specific embodiment of the invention concerns compounds of formula (I-c1):

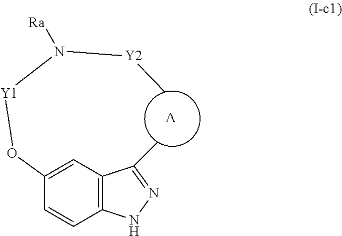

wherein Y1, Y2, Ra and A are as defined for formula (I).

Another preferred specific embodiment of the invention concerns compounds of formula (I-c1a):

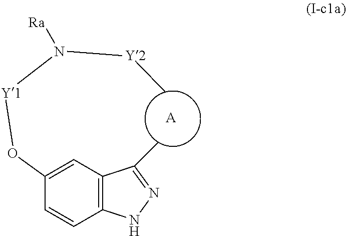

wherein A and Ra are as defined for formula (I), and Y′1 and Y′2, identical or different, each represents a linear or branched (C₂-C₆) alkanediyl group unsubstituted or substituted with one or more groups selected from halogen atoms and alkoxy group.

Another preferred specific embodiment of the invention concerns compounds of formula (I-c1b):

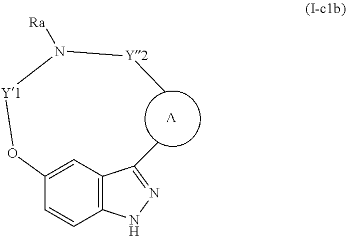

wherein A and Ra are as defined for formula (I), Y′1 represents a linear or branched (C₂-C₆) alkanediyl group unsubstituted or substituted with one or more groups selected from halogen atoms and alkoxy group, and Y″2 represents a linear or branched (C₁-C₅) alkanediyl group unsubstituted or substituted with one or more groups selected from halogen atoms and alkoxy group.

In another preferred embodiment the invention concerns compounds of formula (I-d):

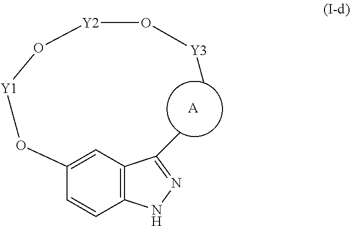

wherein Y1, Y2, Y3 and A are as defined for formula (I).

In another preferred embodiment the invention concerns compounds of formula (I-da):

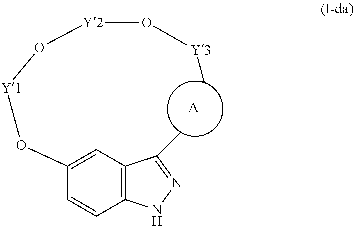

wherein A is as defined for formula (I), Y′1 and Y′2, identical or different, each represent a linear or branched (C₂-C₆) alkanediyl group unsubstituted or substituted with one or more groups selected from halogen atoms and alkoxy group, and Y′3 represents linear or branched (C₁-C₆) alkanediyl group unsubstituted or substituted with one or more groups selected from halogen atoms and alkoxy group.

Another preferred specific embodiment of the invention concerns compounds of formula (I-e):

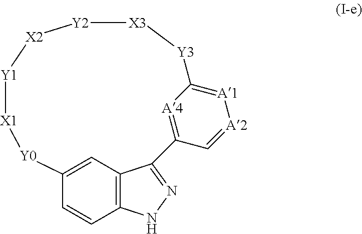

wherein X1, X2, X3, Y0, Y1, Y2, Y3, A′ 1, A′2 and A′4 are as defined for formula (I).

In another preferred embodiment the invention concerns compounds of formula (I-f):

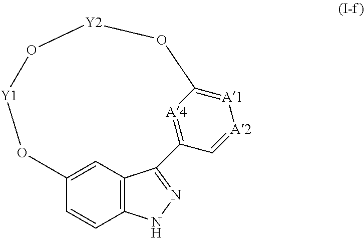

wherein Y1, Y2, A′1, A′2 and A′4 are as defined for formula (I).

In another preferred embodiment the invention concerns compounds of formula (I-fa):

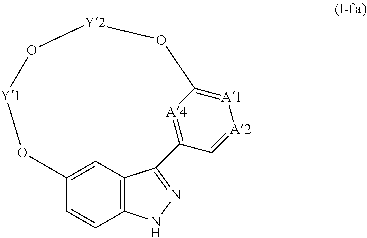

wherein A′ 1, A′2 and A′4 are as defined for formula (I), and Y′1 and Y′2, identical or different, each represents a linear or branched (C₂-C₆) alkanediyl group unsubstituted or substituted with one or more groups selected from halogen atoms and alkoxy group.

In another preferred embodiment the invention concerns compounds of formula (I-fb):

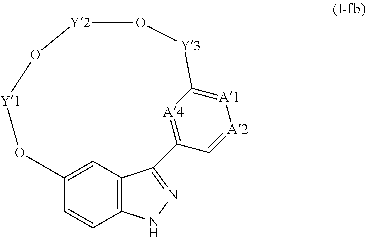

wherein A′ 1, A′2 and A′4 are as defined for formula (I), and Y′1 and Y′2, identical or different, each represents a linear or branched (C₂-C₆) alkanediyl group unsubstituted or substituted with one or more groups selected from halogen atoms and alkoxy group, and Y′3 represents a linear or branched (C₁-C₆) alkanediyl group unsubstituted or substituted with one or more groups selected from halogen atoms and alkoxy group.

In another preferred embodiment, compounds of the invention are compounds of formula (I-f1):

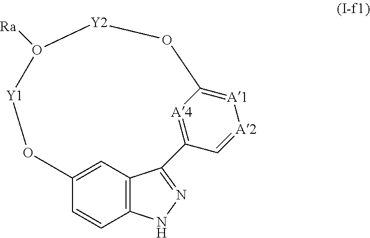

wherein Y1, Y2, A′1, A′2 and A′4 are as defined for formula (I).

In another preferred embodiment, compounds of the invention are compounds of formula (I-f1a):

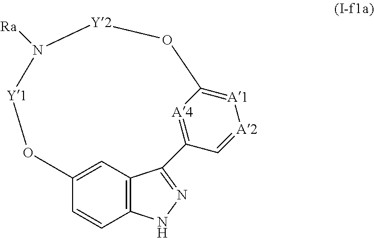

wherein Ra, A′1, A′2 and A′4 are as defined for formula (I), and Y′1 and Y′2, identical or substituted with one or more groups selected from halogen atoms and alkoxy group.

In another preferred embodiment, compounds of the invention are compounds of formula (I-f1b):

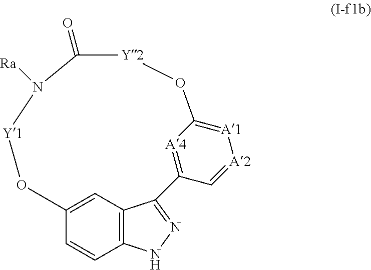

wherein Ra, A′ 1, A′2 and A′4 are as defined for formula (I), Y′1 represents a linear or branched (C₂-C₆) alkanediyl group unsubstituted or substituted with one or more groups selected from halogen atoms and alkoxy group, and Y″2 represents a linear or branched (C₁—C) alkanediyl group unsubstituted or substituted with one or more groups selected from halogen atoms and alkoxy group.

In another preferred embodiment, compounds of the invention are compounds of formula (I-f1c):

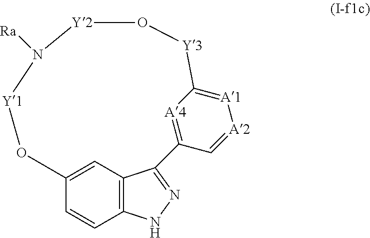

wherein Ra, A′1, A′2 and A′4 are as defined for formula (I), Y′1 and Y′2, identical or different, each represents a linear or branched (C₂-C₆) alkanediyl group unsubstituted or substituted with one or more groups selected from halogen atoms and alkoxy group, and Y′3 represents a linear or branched (C₁-C₆) alkanediyl group unsubstituted or substituted with one or more groups selected from halogen atoms and alkoxy group.

In another preferred embodiment, compounds of the invention are compounds of formula (I-f1d):

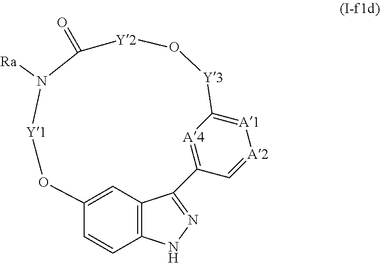

wherein Ra, A′ 1, A′2 and A′4 are as defined for formula (I), Y′1 represents a linear or branched (C₂-C₆) alkanediyl group unsubstituted or substituted with one or more groups selected from halogen atoms and alkoxy group, Y″2 represents a linear or branched (C₁-C₅) alkanediyl group unsubstituted or substituted with one or more groups selected from halogen atoms and alkoxy group, and Y′3 represents a linear or branched (C₁-C₆) alkanediyl group unsubstituted or substituted with one or more groups selected from halogen atoms and alkoxy group.

Another specific embodiment is related to compounds of formula (J-g):

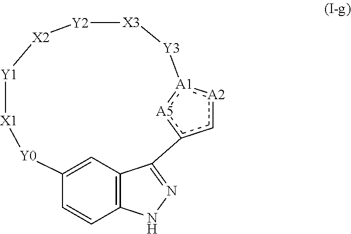

wherein X1, X2, X3, Y0, Y1, Y2, Y3, A1, A2, A5 are as defined for formula (I), and the dotted lines means that the bond can be single or double.

Another specific embodiment is related to compounds of formula (I-h):

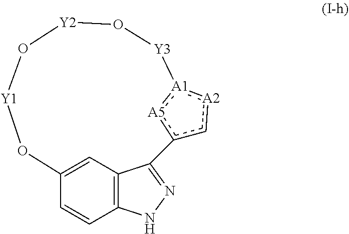

wherein Y1, Y2, Y3, A1, A2, A5 are as defined for formula (I), and the dotted lines means that the bond can be single or double.

Another specific embodiment is related to compounds of formula (I-ha):

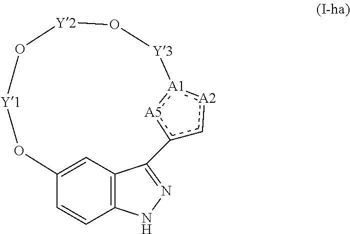

wherein A1, A2, A5 are as defined for formula (I), the dotted lines means that the bond can be single or double, Y′1 and Y′2, identical or different, each represents a linear or branched (C₂-C₆) alkanediyl group unsubstituted or substituted with one or more groups selected from halogen atoms and alkoxy group, and Y′3 represents a linear or branched (C₁-C₆) alkanediyl group unsubstituted or substituted with one or more groups selected from halogen atoms and alkoxy group.

Another specific embodiment is related to compounds of formula (I-h1):

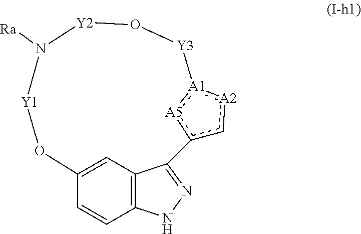

wherein Y1, Y2, Y3, Ra, A1, A2, A5 are as defined for formula (I), and the dotted lines means that the bond can be single or double.

Another specific embodiment is related to compounds of formula (I-h1a):

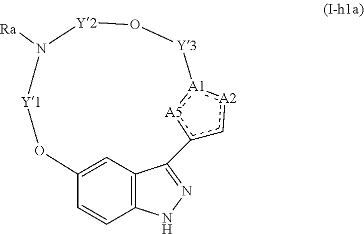

wherein Ra, A1, A2, A5 are as defined for formula (I), the dotted lines means that the bond can be single or double, Y′1 and Y′2, identical or different, each represents a linear or branched (C₂-C₆) alkanediyl group unsubstituted or substituted with one or more groups selected from halogen atoms and alkoxy group, and Y′3 represents a linear or branched (C₁-C₆) alkanediyl group unsubstituted or substituted with one or more groups selected from halogen atoms and alkoxy group.

Another specific embodiment is related to compounds of formula (I-h1b):

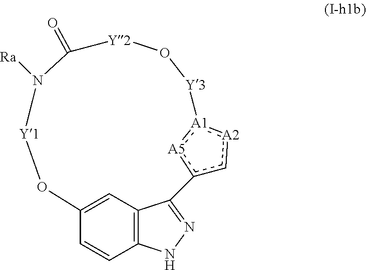

wherein Ra, A1, A2, A5 are as defined for formula (I), the dotted lines means that the bond can be single or double, Y′1 represents a linear or branched (C₂-C₆) alkanediyl group unsubstituted or substituted with one or more groups selected from halogen atoms and alkoxy group, Y″2 represents a linear or branched (C₁-C₅) alkanediyl group unsubstituted or substituted with one or more groups selected from halogen atoms and alkoxy group, and Y′3 represents a linear or branched (C₁-C₆) alkanediyl group unsubstituted or substituted with one or more groups selected from halogen atoms and alkoxy group.

Another specific embodiment is related to compounds of formula (I-i):

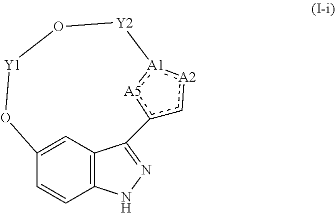

wherein Y1, Y2, A1, A2, A5 are as defined for formula (I), and the dotted lines means that the bond can be single or double.

Another specific embodiment is related to compounds of formula (I-ia):

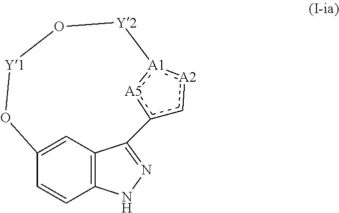

wherein A1, A2, A5 are as defined for formula (I), the dotted lines means that the bond can be single or double, and Y′1 and Y′2, identical or different, each represents a linear or branched (C₂-C₆) alkanediyl group unsubstituted or substituted with one or more groups selected from halogen atoms and alkoxy group.

Another specific embodiment is related to compounds of formula (I-i1):

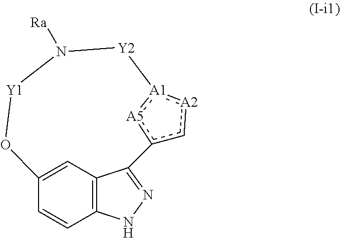

wherein Y1, Y2, Ra, A1, A2, A5 are as defined for formula (I), and the dotted lines means that the bond can be single or double.

Another specific embodiment is related to compounds of formula (I-i1a):

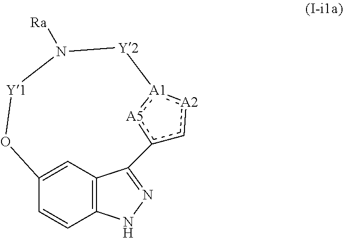

wherein Ra, A1, A2, A5 are as defined for formula (I), the dotted lines means that the bond can be single or double, and Y′1 and Y′2, identical or different, each represents a linear or branched (C₂-C₆) alkanediyl group unsubstituted or substituted with one or more groups selected from halogen atoms and alkoxy group.

Another specific embodiment is related to compounds of formula (I-i1b):

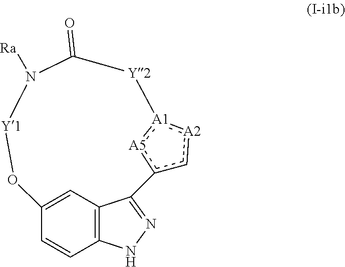

wherein Ra, A1, A2, A5 are as defined for formula (I), the dotted lines means that the bond can be single or double, Y′1 represents a linear or branched (C₂-C₆) alkanediyl group unsubstituted or substituted with one or more groups selected from halogen atoms and alkoxy group, and Y′3 represents a linear or branched (C₁-C₆) alkanediyl group unsubstituted or substituted with one or more groups selected from halogen atoms and alkoxy group.

Another specific embodiment of the invention concerns compounds of formula (I) for which the —Y0-X1-Y1-X2-Y2-X3-Y3- chain represents preferentially the followings, to be read in the same direction from left to right:
—O—(CH₂)₂—O—(CH₂)₂—O—(CH₂)₂—,
—O—CH₂—CH(Me)-O—(CH₂)₂—O—(CH₂)₂—,
—O—CH(Me)-CH₂—O—(CH₂)₂—O—(CH₂)₂—,
—O—(CH₂)₂—O—(CH₂)₂O—CH(Me)-CH₂—,
—O—(CH₂)₂—O—(CH₂)₂O—CH₂—CH(Me)-,
—O—CH₂—CH(Me)-O—(CH₂)₂O—CH₂—CH(Me)-,
—O—(CH₂)₂O—CH(Me)-CH₂—O—(CH₂)₂—,
—O—CH(Me)-CH₂—O—(CH₂)₂O—CH₂—CH(Me)-,
—O—CH(Me)-CH₂—O—(CH₂)₂O—CH₂—CH(F)—,
—O—CH(Me)-CH(Me)-O—(CH₂)₂—O—(CH₂)₂—,
—O—CF₂—CH₂—O—(CH₂)₂—O—(CH₂)₂—,
—O—(CH₂)₃—O—(CH₂)₃—,
—O—CH₂—CF₂—CH₂—O—(CH₂)₃—,
—O—CH(Me)-(CH₂)₂—O—(CH₂)₃—,
—O—CH(Me)-CH₂—CH(Me)-O—(CH₂)₃—,
—O—(CH₂)₃O—CH(Me)-CH₂—CH(Me)-,
—O—CH(Me)-(CH₂)₂—O—(CD₂)₃-,
—O—CH(Me)-(CH₂)₂—O—(CH₂)₂—CH(Me)-,
—O—CH(Me)-(CH₂)₂O—CH(Me)-(CH₂)₂,
—O—CH(Me)-(CH₂)₂O—CH₂—CH(Me)-CH₂—,
—O—(CH₂)₃O—CH(Me)-(CH₂)₂—,
—O—(CH₂)₃O—CH₂—C(Me)₂-CH₂—,
—O—(CH₂)₃—O—(CH₂)₂—CH(Me)-,
—O—CH(Me)-CH₂—CH(Me)-O—CH(Me)-(CH₂)₂—,
—O—CH(Me)-CH₂—O—(CH₂)₄—,
—O—(CH₂)₃—O—(CH₂)₂—O—,
—O—CH(Me)-(CH₂)₂O—CH₂—CH(Me)-O—,
—O—CH(Me)-(CH₂)₂O—CH(Me)-CH₂—O—,
—O—CH(Me)-(CH₂)₂—O—(CH₂)₂—O—,
—O—(CH₂)₃O—CH₂—CH(Me)-O—,
—O—(CH₂)₂—O—(CH₂)₃—O—,
—O—CH(Me)-CH₂—O—(CH₂)₃—O—,
—O—CH(Me)-CH₂—O—(CH₂)₂—CH(Me)-O—,
—O—(CH₂)₃—O—(CH₂)₄—O—,
—O—CH(Me)-(CH₂)₂—O—(CH₂)₂—O—CH₂—,
—O—CH(Me)-CH₂—O—(CH₂)₂O—CH(Me)-,
—O—(CH₂)₂—O—(CH₂)₃—O—CH₂—,
—O—CH(Me)-(CH₂)₂—O—(CH₂)₂—O—(CH₂)₂—.

Alternatively, another preferred —Y0-X1-Y1-X2-Y2-X3-Y3- chain is represented by the followings, to be read in the same direction from left to right:
—O—(CH₂)₃—NHC(O)—CH₂—O—,
—O—(CH₂)₃—N(Me)C(O)—CH₂—O—,
—O—(CH₂)₂—NHC(O)—CH₂—O—,
—O—CH(Me)-(CH₂)₂—NHC(O)—CH₂—O—,
—O—CH₂—CF₂—CH₂—NHC(O)—CH₂—O—,
—O—CH₂—(CH(Me))₂—NHC(O)—CH₂—O—,
—O—(CH₂)₃—NHC(O)—CH(Me)-O—,
—O—CH₂—CHF—CH(Me)-NHC(O)—CH₂—O—,
—O—(CH₂)₃—NHC(O)—(CH₂)₂—,
—O—CH(Me)-(CH₂)₂—NHC(O)—(CH₂)₂—,
—O—CH₂—CF₂—CH₂—NHC(O)—(CH₂)₂—,
—O—CH₂—CHF—CH₂—NHC(O)—(CH₂)₂—,
—O—CH(Me)-(CH₂)₂—NHC(O)—CH₂—CH(Me)-,
—O—(CH₂)₃—NH—(CH₂)₂—O—,
—O—(CH₂)₂—NH—(CH₂)₃—O—,
—O—(CH₂)₃—NH—CH₂—CH(Me)-O—,
—O—CH(Me)-(CH₂)₂—NH—(CH₂)₂—O—,
—O—CH(Me)-CH₂—NH—(CH₂)₃—O—,
—O—CH(Me)-CH₂—NH—(CH₂)₂—CH(Me)O—,
—O—CH(Me)-(CH₂)₂—NH—CH₂—CH(Me)-O—,
—O—CH₂—(CH(Me))₂—NH—(CH₂)₂—O—,
—O—CH₂—CHF—CH(Me)-NH—(CH₂)₂—O—,
—O—(CH₂)₂—CH(Me)-NH—(CH₂)₂—O—,
—O—(CH₂)₃—NH—(CH₂)₃—,
—O—CH(Me)-(CH₂)₂—NH—(CH₂)₃—,
—O—CH(Me)-(CH₂)₂—NH—(CH₂)₂—CH(Me)-
—O—CH(Me)-(CH₂)₂—N(Me)-CH₂—CH(Me)-.

Preferentially compounds of formula (I) of the present invention are:
4-(morpholin-4-yl)-7,10-dioxa-13,18,19-triazatetracyclo[12.5.2.1^{2,6}.0^{17,20}]docosa-1(19), 2(22),3,5,14(21),15,17(20)-heptaene
7,10,14-trioxa-4,19,20-triazatetracyclo[13.5.2.1^{2,6}.0^{18,21}]tricosa-1(20),2(23),3,5,15(22), 16,18(21)-heptaene
4-(morpholin-4-yl)-7,11,14-trioxa-19,20-diazatetracyclo[13.5.2.1^{2,6}.0^{18,21}]tricosa-1(20), 2(23),3,5,15(22),16,18(21)-heptaene
7,10-dioxa-4,14,19,20-tetraazatetracyclo[13.5.2.1^{2,6}.0^{18,21}]tricosa-1(20),2(23),3,5,15(22), 16,18(21)-heptaene
9,13-dioxa-4,5,18,19-tetraazatetracyclo[12.5.2.1^{2,5}.0^{17,20}]docosa-1(19),2(22),3,14(21),15, 17(20)-hexaene
10,14-dioxa-4,5,19,20-tetraazatetracyclo[13.5.2.1^{2,5}.0^{18,21}]tricosa-1(20),2(23),3,15(22),16, 18(21)-hexaene
(13R)-13-methyl-7,11,14-trioxa-5,19,20,23-tetraazatetracyclo[13.5.2.1^{2,6}.0^{18,21}]tricosa-1(20),2(23),3,5,15(22),16,18(21)-heptaene
(6R)-6-methyl-9,13-dioxa-4,5,18,19-tetraazatetracyclo[12.5.2.1^{2,5}.0^{17,20}]docosa-1(19), 2(22),3,14(21),15,17(20)-hexaene
(7S,13S)-7,13-dimethyl-8,11,14-trioxa-4,19,20-triazatetracyclo[13.5.2.1^{2,6}.0^{18,21}]tricosa-1(20),2(23),3,5,15(22),16,18(21)-heptaene
(7R,13S)-7,13-dimethyl-8,11,14-trioxa-4,19,20-triazatetracyclo[13.5.2.1^{2,6}.0^{18,21}]tricosa-1(20),2(23),3,5,15(22),16,18(21)-heptaene
(8S,13R)-8,13-dimethyl-7,11,14-trioxa-4,19,20-triazatetracyclo[13.5.2.1^{2,6}.0^{18,21}]tricosa-1(20),2(23),3,5,15(22),16,18(21)-heptaene
(8S,13R)-8,13-dimethyl-7,11,14-trioxa-4,19,20-triazatetracyclo[13.5.2.1^{2,6}.0^{18,21}]tricosa-1(20),2(23),3,5,15(22),16,18(21)-heptaene
(7R,13R)-7,13-dimethyl-8,11,14-trioxa-4,19,20-triazatetracyclo[13.5.2.1^{2,6}.0^{18,21}]tricosa-1(20),2(23),3,5,15(22),16,18(21)-heptaene hydrochloride
(7S,13R)-7,13-dimethyl-8,11,14-trioxa-4,19,20-triazatetracyclo[13.5.2.1^{2,6}.0^{18,21}]tricosa-1(20),2(23),3,5,15(22),16,18(21)-heptaene hydrochloride
(6S)-6-methyl-9,13-dioxa-4,5,18,19-tetraazatetracyclo[12.5.2.1^{2,5}.0^{17,20}]docosa-1(19), 2(22),3,14(21),15,17(20)-hexaene
(13S)-13-methyl-7,10,14-trioxa-5,19,20,23-tetraazatetracyclo[13.5.2.1^{2,6}.0^{18,21}]tricosa-1(20),2(23),3,5,15(22),16,18(21)-heptaene
(8R,13R)-8,13-dimethyl-7,11,14-trioxa-4,19,20-triazatetracyclo[13.5.2.1^{2,6}.0^{18,21}]tricosa-1(20),2(23),3,5,15(22),16,18(21)-heptaene
(8R,13S)-8,13-dimethyl-7,11,14-trioxa-4,19,20-triazatetracyclo[13.5.2.1^{2,6}.0^{18,21}]tricosa-1(20),2(23),3,5,15(22),16,18(21)-heptaene
(13S)-13-methyl-4-(pyrrolidin-1-yl)-7,11,14-trioxa-5,19,20,23-tetraazatetracyclo [13.5.2.1^{2,6}.0^{18,21}]tricosa-1(20),2(23),3,5,15(22),16,18(21)heptaene
(8S,13S)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene
(8R,13S)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene
(8S,13R)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene
(8R,13R)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene
(13S)-13-methyl-8,11,14-trioxa-4,5,19,20-tetraazatetracyclo[13.5.2.1^{2,5}.0^{18,21}]tricosa-1(20),2(23),3,15(22),16,18(21)-hexaene
(6R,12S)-6,12-dimethyl-9,13-dioxa-4,5,18,19-tetraazatetracyclo[12.5.2.1^{2,5}.0^{17,20}]docosa-1(19),2(22),3,14(21),15,17(20)-hexaene
(13S)-13-methyl-7,11,14-trioxa-5,19,20,23-tetraazatetracyclo[13.5.2.1^{2,6}.0^{18,21}]tricosa-1(20),2(23),3,5,15(22),16,18(21)-heptaene
(13S)-4-(3-methoxyazetidin-1-yl)-13-methyl-7,11,14-trioxa-5,19,20,23-tetraazatetracyclo [13.5.2.1^{2,6}.0^{18,21}]tricosa-1(20),2(23),3,5,15(22),16,18(21)heptaene
(13S)-13-methyl-7,10,14-trioxa-19,20,23-triazatetracyclo[13.5.2.1^{2,6}.0^{18,21}]tricosa-1(20), 2(23),3,5,15(22),16,18(21)-heptaene
(13S)-4,13-dimethyl-7,11,14-trioxa-5,19,20,23-tetraazatetracyclo[13.5.2.1^{2,6}.0^{18,21}]tricosa-1(20),2(23),3,5,15(22),16,18(21)-heptaene
(13S)-13-methyl-7,10-dioxa-5,14,19,20,23-pentaazatetracyclo[13.5.2.1^{2,6}.0^{18,21}]tricosa-1(20),2(23),3,5,15(22),16,18(21)-heptaene
7,7-dimethyl-9,13-dioxa-4,5,18,19-tetraazatetracyclo[12.5.2.1^{2,5}.0^{17,20}]docosa-1(19),2(22), 3,14(21),15,17(20)-hexaene
(12S)-12-methyl-9,13-dioxa-4,5,18,19-tetraazatetracyclo[12.5.2.1^{2,5}.0^{17,20}]docosa-1(19), 2(22),3,14(21),15,17(20)-hexaene
(13S)-13-methyl-7,10,14-trioxa-19,20,23-triazatetracyclo[13.5.2.1^{2,6}.0^{18,21}]tricosa-1(20), 2(23),3,5,15(22),16,18(21)-heptaene
(13S)-4-(3-methoxyazetidin-1-yl)-13-methyl-7,10-dioxa-5,14,19,20,23-pentaazatetracyclo [13.5.2.1^{2,6}.0^{18,21}]tricosa-1(20),2,4,6(23),15(22),16,18(21)-heptaene
(6R,12R)-6,12-dimethyl-9,13-dioxa-4,5,18,19-tetraazatetracyclo[12.5.2.1^{2,5}.0^{17,20}]docosa-1(19),2(22),3,14(21),15,17(20)-hexaene
(6S,12R)-6,12-dimethyl-9,13-dioxa-4,5,18,19-tetraazatetracyclo[12.5.2.1^{2,5}.0^{17,20}]docosa-1(19),2(22),3,14(21),15,17(20)-hexaene
(6S)-6-methyl-9,13-dioxa-4,5,18,19-tetraazatetracyclo[12.5.2.1^{2,5}.0^{17,20}]docosa-1(19), 2(22),3,14(21),15,17(20)-hexaene
(6S,12S)-6,12-dimethyl-9,13-dioxa-4,5,18,19-tetraazatetracyclo[12.5.2.1^{2,5}.0^{17,20}]docosa-1(19),2(22),3,14(21),15,17(20)-hexaene
(13S)-13-methyl-4-(pyrrolidin-1-yl)-7,10-dioxa-5,14,19,20,23-pentaazatetracyclo [13.5.2.1^{2,6}.0^{18,21}]tricosa-1(20),2(23),3,5,15(22),16,18(21)-heptaene
(13S)-4,13-dimethyl-7,10-dioxa-5,14,19,20,23-pentaazatetracyclo[13.5.2.1^{2,6}.0^{18,21}] tricosa-1(20),2(23),3,5,15(22),16,18(21)-heptaene
(13R)-13-methyl-7,10,14-trioxa-4,19,20,23-tetraazatetracyclo[13.5.2.1^{2,6}.0^{18,21}]tricosa-1(20),2(23),3,5,15(22),16,18(21)-heptaene
(12R)-12-methyl-9,13-dioxa-4,5,18,19-tetraazatetracyclo[12.5.2.1^{2,5}.0^{17,20}]docosa-1(19), 2(22),3,14(21),15,17(20)-hexaene
(13S)-13-methyl-10,14-dioxa-19,20-diazatetracyclo[13.5.2.1^{2,6}.0^{18,21}]tricosa-1(20),2(23), 3,5,15(22),16,18(21)-heptaene
(8R,13S)-4-(3-methoxyazetidin-1-yl)-8,13-dimethyl-7,10,14-trioxa-5,19,20,23-tetraaza tetracyclo[13.5.2.1^{2,6}.0^{18,21}]tricosa-1(20),2(23),3,5,15(22),16,18(21)-heptaene
(13R)-13-methyl-7,10,14-trioxa-5,19,20,23-tetraazatetracyclo[13.5.2.1^{2,6}.0^{18,21}]tricosa-1(20),2(23),3,5,15(22),16,18(21)-heptaene
(8S,13S)-8,13-dimethyl-7,10,14-trioxa-5,19,20,23-tetraazatetracyclo[13.5.2.1^{2,6}.0^{18,21}] tricosa-1(20),2(23),3,5,15(22),16,18(21)-heptaene
(8S,13S)-8,13-dimethyl-7,10,14-trioxa-4,19,20,23-tetraazatetracyclo[13.5.2.1^{2,6}.0^{18,21}] tricosa-1(20),2(23),3,5,15(22),16,18(21)-heptaene
(12S)-12-methyl-9,13-dioxa-3-thia-18,19,22-triazatetracyclo[12.5.2.1^{2,5}.0^{17,20}]docosa-1(19),2(22),4,14(21),15,17(20)-hexaene
(8R,13S)-4-[(3R)-3-methoxypyrrolidin-1-yl]-8,13-dimethyl-7,10,14-trioxa-5,19,20,23-tetraazatetracyclo[13.5.2.1^{2,6}.0^{18,21}]tricosa-1(20),2(23),3,5,15(22),16,18(21)-heptaene
(13R)-13-methyl-8,11,14-trioxa-4,5,19,20-tetraazatetracyclo[13.5.2.1^{2,5}.0^{18,21}]tricosa-1(20),2(23),3,15(22),16,18(21)-hexaene
(9S,13S)-9,13-dimethyl-7,10,14-trioxa-4,19,20,23-tetraazatetracyclo[13.5.2.1^{2,6}.0^{18,21}] tricosa-1(20),2(23),3,5,15(22),16,18(21)-heptaene
(7R,12S)-7,12-dimethyl-9,13-dioxa-4,5,18,19-tetraazatetracyclo[12.5.2.1^{2,5}.0^{17,20}]docosa-1(19),2(22),3,14(21),15,17(20)-hexaene
(8R,13S)-8,13-dimethyl-7,11,14-trioxa-5,19,20,23-tetraazatetracyclo[13.5.2.1^{2,6}.0^{18,21}] tricosa-1(20),2(23),3,5,15(22),16,18(21)-heptaene
(12S)-12-methyl-8,11,14-trioxa-4,5,19,20-tetraazatetracyclo[13.5.2.1^{2,5}.0^{18,21}]tricosa-1(20),2(23),3,15(22),16,18(21)-hexaene
(8R)-8-methyl-7,10,14-trioxa-5,19,20,23-tetraazatetracyclo[13.5.2.1^{2,6}.0^{18,21}]tricosa-1(20) 2(23),3,5,15(22),16,18(21)heptaene
(13R)-13-methyl-7,10,14-trioxa-19,20,23-triazatetracyclo[13.5.2.1^{2,6}.0^{18,21}]tricosa-1(20), 2(23),3,5,15(22),16,18(21)-heptaene
(12S)-12-methyl-9,13-dioxa-3,5,18,19-tetraazatetracyclo[12.5.2.1^{2,5}.0^{17,20}]docosa-1(19), 2(22),3,14(21),15,17(20)-hexaene
(9R,13S)-9,13-dimethyl-7,10,14-trioxa-4,19,20,23-tetraazatetracyclo[13.5.2.1^{2,6}.0^{18,21}] tricosa-1(20),2(23),3,5,15(22),16,18(21)-heptaene
(9S,13S)-9,13-dimethyl-7,10,14-trioxa-5,19,20,23-tetraazatetracyclo[13.5.2.1^{2,6}.0^{18,21}] tricosa-1(20),2(23),3,5,15(22),16,18(21)-heptaene
(9R)-9-methyl-8,11,14-trioxa-4,5,19,20-tetraazatetracyclo[13.5.2.1^{2,5}.0^{18,21}]tricosa-1(20), 2(23),3,15(22),16,18(21)-hexaene
(9S)-9-methyl-8,11,14-trioxa-4,5,19,20-tetraazatetracyclo[13.5.2.1^{2,5}.0^{18,21}]tricosa-1(20), 2(23),3,15(22),16,18(21)-hexaene
(13S)-13-methyl-7,10,14-trioxa-4-thia-19,20,23-triazatetracyclo[13.5.2.1^{2,5}.0^{18,21}]tricosa-1(20),2,5(23),15(22),16,18(21)-hexaene
8,11,14-trioxa-4,5,19,20-tetraazatetracyclo[13.5.2.1^{2,5}.0^{18,21}]tricosa-1(20),2(23),3,15(22), 16,18(21)-hexaene
(6S)-6-methyl-8,11,14-trioxa-4,5,19,20-tetraazatetracyclo[13.5.2.1^{2,5}.0^{18,21}]tricosa-1(20), 2(23),3,15(22),16,18(21)-hexaene
(6R)-6-methyl-8,11,14-trioxa-4,5,19,20-tetraazatetracyclo[13.5.2.1^{2,5}.0^{18,21}]tricosa-1(20), 2(23),3,15(22),16,18(21)-hexaene
(13S)-13-methyl-7,10,14-trioxa-23-thia-4,19,20-triazatetracyclo[13.5.2.1^{2,5}.0^{18,21}]tricosa-1(20),2,4,15(22),16,18(21)-hexaene
(12R)-12-methyl-8,11,14-trioxa-4,5,19,20-tetraazatetracyclo[13.5.2.1^{2,5}.0^{18,21}]tricosa-1(20),2(23),3,15(22),16,18(21)-hexaene
9,12,15-trioxa-4,20,21-triazatetracyclo[14.5.2.1^{2,6}.0^{19,22}]tetracosa-1(21),2(24),3,5,16(23), 17,19(22)-heptaene
(14S)-14-methyl-9,12,15-trioxa-4,20,21-triazatetracyclo[14.5.2.1^{2,6}.0^{19,22}]tetracosa-1(21), 2(24),3,5,16(23),17,19(22)-heptaene
(10R)-10-methyl-8,11,14-trioxa-4,5,19,20-tetraazatetracyclo[13.5.2.1^{2,5}.0^{18,21}]tricosa-1(20),2(23),3,15(22),16,18(21)-hexaene
(13R)-13-methyl-8,11,14-trioxa-4,5,19,20,23-pentaazatetracyclo[13.5.2.1^{2,5}.0^{18,21}]tricosa-1(20),2(23),3,15(22),16,18(21)-hexaene
(13S)-13-methyl-8,11,14-trioxa-5,19,20-triazatetracyclo[13.5.2.1^{2,5}.0^{18,21}]tricosa-1(20),2(23),3,15(22),16,18(21)-hexaene-4-carbonitrile
8,11,14-trioxa-4,5,19,20,22-pentaazatetracyclo[13.5.2.1^{2,5}.0^{18,21}]tricosa-1(20),2(23),3,15(22),16,18(21)-hexaene
9,13-dioxa-4,5,18,19,22-pentaazatetracyclo[12.5.2.1^{2,5}.0^{17,20}]docosa-1(19),2(22),3,14(21), 15,17(20)-hexaene
(13R)-13-methyl-7,10,14-trioxa-23-thia-4,19,20-triazatetracyclo[13.5.2.1^{2,6}.0^{18,21}]tricosa-1(20),2,4,15(22),16,18(21)-hexaene
(13R)-4,13-dimethyl-8,11,14-trioxa-4,19,20,23-tetraazatetracyclo[13.5.2.1^{2,5}.0^{18,21}]tricosa-1(20),2,5(23),15(22),16,18(21)-hexaene
(7R)-7-methyl-8,11,14-trioxa-4,5,19,20-tetraazatetracyclo[13.5.2.1^{2,5}.0^{18,21}]tricosa-1(20), 2(23),3,15(22),16,18(21)-hexaene
(13,13-difluoro-8,11,14-trioxa-4,5,19,20-tetraazatetracyclo[13.5.2.1^{2,5}.0^{18,21}]tricosa-1(20), 2(23),3,15(22),16,18(21)-hexaene
(13S)-4,13-dimethyl-7,10,14-trioxa-4,19,20,23-tetraazatetracyclo[13.5.2.1^{2,5}.0^{18,21}]tricosa-1(20),2,5(23),15(22),16,18(21)-hexaene
(13S)-13-methyl-8,11,14-trioxa-5,19,20,23-tetraazatetracyclo[13.5.2.1^{2,5}.0^{18,21}]tricosa-1(20),2(23),3,15(22),16,18(21)-hexaene
(13S)-13-methyl-8,11,14-trioxa-4,5,19,20,23-pentaazatetracyclo[13.5.2.1^{2,5}.0^{18,21}]tricosa-1(20),2(23),3,15(22),16,18(21)-hexaene
(13R)-13-methyl-8,11,14-trioxa-5,19,20-triazatetracyclo[13.5.2.1^{2,5}.0^{18,21}]tricosa-1(20), 2(23),3,15(22),16,18(21)-hexaene-4-carbonitrile
(13R)-13-methyl-8,11,14-trioxa-5,19,20,23-tetraazatetracyclo[13.5.2.1^{2,5}.0^{18,21}]tricosa-1(20),2(23),3,15(22),16,18(21)-hexaene
(13S)-4,13-dimethyl-7,10,14-trioxa-4,19,20,23-tetraazatetracyclo[13.5.2.1^{2,5}.0^{18,21}]tricosa-1(20),2,5(23),15(22),16,18(21)-hexaene
(12S)-12-methyl-8,11,14-trioxa-5,19,20-triazatetracyclo[13.5.2.1^{2,6}.0^{18,21}]tricosa-1(20), 2(23),3,15(22),16,18(21)-hexaene-4-carbonitrile
(12R)-12-methyl-8,11,14-trioxa-5,19,20-triazatetracyclo[13.5.2.1^{2,6}.0^{18,21}]tricosa-1(20), 2(23),3,15(22),16,18(21)-hexaene-4-carbonitrile
(6R,12S)-6,12-dimethyl-8,11,14-trioxa-4,5,19,20-tetraazatetracyclo[13.5.2.1^{2,5}.0^{18,21}] tricosa-1(20),2(23),3,15(22),16,18(21)-hexaene
(13R)-17-fluoro-13-methyl-8,11,14-trioxa-4,5,19,20-tetraazatetracyclo[13.5.2.1^{2,5}.0^{18,21}] tricosa-1(20),2(23),3,15(22),16,18(21)-hexaene
(6S,12S)-6,12-dimethyl-8,11,14-trioxa-4,5,19,20-tetraazatetracyclo[13.5.2.1^{2,5}.0^{18,21}] tricosa-1(20),2(23),3,15(22),16,18(21)-hexaene
(12R)-12-methyl-9,13-dioxa-4,5,18,19,22-pentaazatetracyclo[12.5.212, 0^{17,20}]docosa-1(19),2(22),3,14(21),15,17(20)-hexaene
13-methyl-4-(morpholin-4-yl)-7,10-dioxa-13,19,20-triazatetracyclo[13.5.2.1^{2,6}.0^{18,21}] tricosa-1(20),2,4,6(23),15,17,21-heptaene
(12S)-12-methyl-9,13-dioxa-4,5,18,19,22-pentaazatetracyclo[12.5.2.1^{2,5}.0^{17,20}]docosa-1(19),2(22),3,14(21),15,17(20)-hexaene
(6S,13R)-6,13-dimethyl-8,11,14-trioxa-4,5,19,20-tetraazatetracyclo[13.5.2.1^{2,5}.0^{18,21}] tricosa-1(20),2(23),3,15(22),16,18(21)-hexaene
(6R,12R)-6,12-dimethyl-9,13-dioxa-4,5,18,19,22-pentaazatetracyclo[12.5.2.1^{2,5}.0^{17,20}] docosa-1(19),2(22),3,14(21),15,17(20)-hexaene
(13R)-6-methoxy-13-methyl-8,11,14-trioxa-4,5,19,20-tetraazatetracyclo[13.5.2.1^{2,5}.0^{18,21}] tricosa-1(20),2(23),3,15(22),16,18(21)-hexaene
8,11,14-trioxa-4,5,16,19,20-pentaazatetracyclo[13.5.2.1^{2,5}.0^{18,21}]tricosa-1(20),2(23),3, 15(22),16,18(21)-hexaene
(8S,12R)-8,12-dimethyl-9,13-dioxa-4,5,18,19-tetraazatetracyclo[12.5.2.1^{2,5}.0^{17,20}]docosa-1(19),2(22),3,14(21),15,17(20)-hexaene
(8R,12R)-8,12-dimethyl-9,13-dioxa-4,5,18,19-tetraazatetracyclo[12.5.2.1^{2,5}.0^{17,20}]docosa-1(19),2(22),3,14(21),15,17(20)-hexaene
(12S,13R)-12,13-dimethyl-8,11,14-trioxa-4,5,19,20-tetraazatetracyclo[13.5.2.1^{2,5}.0^{18,21}] tricosa-1(20),2(23),3,15(22),16,18(21)-hexaene
(12R,13S)-12,13-dimethyl-8,11,14-trioxa-4,5,19,20-tetraazatetracyclo[13.5.2.1^{2,5}.0^{18,21}] tricosa-1(20),2(23),3,15(22),16,18(21)-hexaene
(6S,13S)-6,13-dimethyl-8,11,14-trioxa-4,5,19,20-tetraazatetracyclo[13.5.2.1^{2,5}.0^{18,21}] tricosa-1(20),2(23),3,15(22),16,18(21)-hexaene
8,13-dioxa-4,5,18,19-tetraazatetracyclo[12.5.2.1^{2,5}.0^{17,20}]docosa-1(19),2(22),3,14(21),15, 17(20)-hexaene
(6S,12R)-6,12-dimethyl-9,13-dioxa-4,5,18,19,22-pentaazatetracyclo[12.5.2.1^{2,5}.0^{17,20}] docosa-1(19),2(22),3,14(21),15,17(20)-hexaene
10,13-dioxa-4,5,18,19-tetraazatetracyclo[12.5.2.1^{2,5}.0^{17,20}]docosa-1(19),2(22),3,14(21), 15,17(20)-hexaene
(6R or 6S,13R)-6-fluoro-13-methyl-8,11,14-trioxa-4,5,19,20-tetraazatetracyclo[13.5.2.1^{2,5}.0^{18,21}]tricosa-1(20),2(23),3,15(22),16,18(21)-hexaene
(6S or 6R,13R)-6-fluoro-13-methyl-8,11,14-trioxa-4,5,19,20-tetraazatetracyclo[13.5.2.1^{2,5}.0^{18,21}] tricosa-1(20),2(23),3,15(22),16,18(21)-hexaene
(12S)-12-methyl(9,9,10,10-²H₄)-8,11,14-trioxa-4,5,19,20-tetraazatetracyclo[13.5.2.1^{2,5}.0^{18,21}] tricosa-1(20),2(23),3,15,17,21-hexaene
8,14-dioxa-4,5,19,20-tetraazatetracyclo[13.5.2.1^{2,5}.0^{18,21}]tricosa-1(20),2(23),3,15(22), 16,18(21)-hexaene
(6R)-11,11-difluoro-6-methyl-9,13-dioxa-4,5,18,19-tetraazatetracyclo[12.5.2.1^{2,5}.0^{17,20}] docosa-1(19),2(22),3,14(21),15,17(20)-hexaene
(12S)-12-methyl(6,6,7,7-2H4)-8,11,14-trioxa-4,5,19,20-tetraazatetracyclo[13.5.2.1^{2,5}.0^{18,21}]tricosa-1(20),2(23),3,15(22),16,18(21)-hexaene
11,11-difluoro-9,13-dioxa-4,5,18,19-tetraazatetracyclo[12.5.2.1^{2,5}.0^{17,20}]docosa-1(19),2(22),3,14(21),15,17(20)-hexaene
(12S)-12-methyl-11,14-dioxa-4,5,19,20-tetraazatetracyclo[13.5.2.1^{2,5}.0^{18,21}]tricosa-1(20),2(23),3,15(22),16,18(21)-hexaene
(12R,13R)-12,13-dimethyl-8,11,14-trioxa-4,5,19,20-tetraazatetracyclo[13.5.2.1^{2,5}.0^{18,21}] tricosa-1(20),2(23),3,15(22),16,18(21)-hexaene
(12S,13S)-12,13-dimethyl-8,11,14-trioxa-4,5,19,20-tetraazatetracyclo[13.5.2.1^{2,5}.0^{18,21}] tricosa-1(20),2(23),3,15(22),16,18(21)-hexaene
8,13-dioxa-4,5,18,19,22-pentaazatetracyclo[12.5.2.1^{2,5}.0^{17,20}]docosa-1(19),2(22),3,14(21),15,17(20)-hexaene
(6R,10S)-6,10-dimethyl-9,13-dioxa-4,5,18,19-tetraazatetracyclo[12.5.2.1^{2,5}.0^{17,20}]docosa-1(19),2(22),3,14(21),15,17(20)-hexaene
(6R,8R)-6,8-dimethyl-9,13-dioxa-4,5,18,19-tetraazatetracyclo[12.5.2.1^{2,5}.0^{17,20}]docosa-1(19),2(22),3,14(21),15,17(20)-hexaene
7,7-difluoro-9,13-dioxa-4,5,18,19-tetraazatetracyclo[12.5.2.1^{2,5}.0^{17,20}]docosa-1(19),2(22),3,14(21),15,17(20)-hexaene
(13S)-4,13-dimethyl-7,11,14-trioxa-4,19,20,23-tetraazatetracyclo[13.5.2.1^{2,5}.0^{18,21}]tricosa-1(20),2,5(23),15(22),16,18(21)-hexaene
(12R)-7,7-difluoro-12-methyl-9,13-dioxa-4,5,18,19-tetraazatetracyclo[12.5.2.1^{2,5}.0^{17,20}] docosa-1(19),2(22),3,14(21),15,17(20)-hexaene
(8E)-11,14-dioxa-4,5,19,20-tetraazatetracyclo[13.5.2.1^{2,5}.0^{18,21}]tricosa-1(20),2(23),3,8,15(22),16,18(21)-heptaene
(12S)-12-methyl-8,13-dioxa-4,5,18,19,22-pentaazatetracyclo[12.5.2.1^{2,5}.0^{17,20}]docosa-1(19),2(22),3,14(21),15,17(20)-hexaene
(6R,10R)-6,10-dimethyl-9,13-dioxa-4,5,18,19-tetraazatetracyclo[12.5.2.1^{2,5}.0^{17,20}]docosa-1(19),2(22),3,14(21),15,17(20)-hexaene
(12R)-12-methyl-8,13-dioxa-4,5,18,19,22-pentaazatetracyclo[12.5.2.1^{2,5}.0^{17,20}]docosa-1(19),2(22),3,14(21),15,17(20)-hexaene
(12R)-12-methyl-9,13-dioxa-2,4,18,19,22-pentaazatetracyclo[12.5.2.1^{2,5}.0^{17,20}]docosa-1(19),3,5(22),14(21),15,17(20)-hexaene
(10R,12R)-10,12-dimethyl-9,13-dioxa-4,5,18,19-tetraazatetracyclo[12.5.2.1^{2,5}.0^{17,20}]docosa-1(19),2(22),3,14(21),15,17(20)-hexaene
(8R,12R)-8,12-dimethyl-9,13-dioxa-4,5,18,19,22-pentaazatetracyclo[12.5.2.1^{2,5}.0^{17,20}]docosa-1(19),2(22),3,14(21),15,17(20)-hexaene
(6R,12R)-6,12-dimethyl-8,13-dioxa-4,5,18,19-tetraazatetracyclo[12.5.2.1^{2,5}.0^{17,20}]docosa-1(19),2(22),3,14(21),15,17(20)-hexaene
(13S)-13-methyl-9,12,15-trioxa-4,5,20,21-tetraazatetracyclo[14.5.2.1^{2,5}.0^{19,22}]tetracosa-1(21),2(24),3,16(23),17,19(22)-hexaene
(12R)-12-methyl-10,13-dioxa-4,5,18,19,22-pentaazatetracyclo[12.5.212, 0^{17,20}]docosa-1(19),2(22),3,14(21),15,17(20)-hexaene
(14R)-14-methyl-8,11,15-trioxa-4,5,20,21-tetraazatetracyclo[14.5.2.1^{2,5}.0^{19,22}]tetracosa-1(21),2(24),3,16(23),17,19(22)-hexaene
(6R,8S)-6,8-dimethyl-9,13-dioxa-4,5,18,19-tetraazatetracyclo[12.5.2.1^{2,5}.0^{17,20}]docosa-1(19),2(22),3,14(21),15,17(20)-hexaene
(14R)-14-methyl-8,11,15-trioxa-4,5,20,21,24-pentaazatetracyclo[14.5.2.1^{2,5}.0^{19,22}]tetracosa-1(21),2(24),3,16(23),17,19(22)-hexaene
7,14-dioxa-10,19,20-triazatetracyclo[13.5.2.1^{2,6}.0^{18,21}]tricosa-1(20),2(23),3,5,15(22), 16,18(21)-heptaen-9-one
7,14-dioxa-10,19,20-triazatetracyclo[13.5.2.1^{2,6}.0^{18,21}]tricosa-1(20),2(23),3,5,15(22), 16,18(21)-heptaene
7,14-dioxa-4,10,19,20-tetraazatetracyclo[13.5.2.1^{2,6}.0^{18,21}]tricosa-1(20),2,4,6(23),15, 17,21-heptaen-9-one
7,14-dioxa-4,10,19,20-tetraazatetracyclo[13.5.2.1^{2,6}.0^{18,21}]tricosa-1(20),2,4,6(23),15, 17,21-heptaene
5-fluoro-7,14-dioxa-10,19,20-triazatetracyclo[13.5.2.1^{2,6}.0^{18,21}]tricosa-1(20),2,4,6(23), 15,17,21-heptaen-9-one
5-fluoro-7,14-dioxa-10,19,20-triazatetracyclo[13.5.2.1^{2,6}.0^{18,21}]tricosa-1(20),2,4,6(23), 15,17,21-heptaene
5-methyl-7,14-dioxa-10,19,20-triazatetracyclo[13.5.2.1^{2,6}.0^{18,21}]tricosa-1(20),2,4,6(23),15, 17,21-heptaen-9-one
5-methyl-7,14-dioxa-10,19,20-triazatetracyclo[13.5.2.1^{2,6}.0^{18,21}]tricosa-1(20),2,4,6(23),15, 17,21-heptaene
4-chloro-7,14-dioxa-10,19,20-triazatetracyclo[13.5.2.1^{2,6}.0^{18,21}]tricosa-1(20),2,4,6(23),15, 17,21-heptaen-9-one
4-chloro-7,14-dioxa-10,19,20-triazatetracyclo[13.5.2.1^{2,6}.0^{18,21}]tricosa-1(20),2,4,6(23),15, 17,21-heptaene
4-methyl-7,14-dioxa-10,19,20-triazatetracyclo[13.5.2.1^{2,6}.0^{18,21}]tricosa-1(20),2(23),3,5,15, 17,21-heptaen-9-one
4-methyl-7,14-dioxa-10,19,20-triazatetracyclo[13.5.2.1^{2,6}.0^{18,21}]tricosa-1(20),2(23),3,5,15, 17,21-heptaene
4-(oxan-4-yl)-7,14-dioxa-10,19,20-triazatetracyclo[13.5.2.1^{2,6}.0^{18,21}]tricosa-1(20),2(23), 3,5,15,17,21-heptaen-9-one
4-(oxan-4-yl)-7,14-dioxa-10,19,20-triazatetracyclo[13.5.2.1^{2,6}.0^{18,21}]tricosa-1(20),2(23), 3,5,15,17,21-heptaene
4-(oxan-3-yl)-7,14-dioxa-10,19,20-triazatetracyclo[13.5.2.1^{2,6}.0^{18,21}]tricosa-1(20),2(23), 3,5,15,17,21-heptaen-9-one
4-(oxan-3-yl)-7,14-dioxa-10,19,20-triazatetracyclo[13.5.2.1^{2,6}.0^{18,21}]tricosa-1(20),2(23), 3,5,15,17,21-heptaene
(11S) or (11R)-11-(methoxymethyl)-7,14-dioxa-10,19,20-triazatetracyclo[13.5.2.1^{2,6}.0^{18,21}]tricosa-1(20),2(23),3,5,15,17,21-heptaen-9-one
(11S) or (11R)-11-(methoxymethyl)-7,14-dioxa-10,19,20-triazatetracyclo[13.5.2.1^{2,6}.0^{18,21}]tricosa-1(20),2(23),3,5,15,17,21-heptaene
(11R) or (11S)-11-(methoxymethyl)-7,14-dioxa-10,19,20-triazatetracyclo[13.5.2.1^{2,6}.0^{18,21}]tricosa-1(20),2(23),3,5,15,17,21-heptaen-9-one
(11R) or (11S)-11-(methoxymethyl)-7,14-dioxa-10,19,20-triazatetracyclo[13.5.2.1^{2,6}.0^{18,21}]tricosa-1(20),2(23),3,5,15,17,21-heptaene
11-(fluoromethyl)-7,14-dioxa-10,19,20-triazatetracyclo[13.5.2.1^{2,6}.0^{18,21}]tricosa-1(20),2,4,6(23),15,17,21-heptaen-9-one
11-(fluoromethyl)-7,14-dioxa-10,19,20-triazatetracyclo[13.5.2.1^{2,6}.0^{18,21}]tricosa-1(20),2,4,6(23),15,17,21-heptaene
7′,14′-dioxa-4′,10′,19′,20′-tetraazaspiro[cyclopropane-1,8′-tetracyclo[13.5.2.1^{2,6}.0^{18,21}] tricosane]-1′(20′),2′(23′),3′,5′,15′,17′,21′-heptaen-9′-one
7′,14′-dioxa-4′,10′,19′,20′-tetraazaspiro[cyclopropane-1,8′-tetracyclo[13.5.2.1^{2,6}.0^{18,21}] tricosane]-1′(20′),2′(23′),3′,5′,15′,17′,21′-heptaene
13-methyl-7,14-dioxa-4,10,19,20-tetraazatetracyclo[13.5.2.1^{2,6}.0^{18,21}]tricosa-1(20),2,4, 6(23),15,17,21-heptaen-9-one
13-methyl-7,14-dioxa-4,10,19,20-tetraazatetracyclo[13.5.2.1^{2,6}.0^{18,21}]tricosa-1(20),2,4, 6(23),15,17,21-heptaene
8,10-dimethyl-7,14-dioxa-4,10,19,20-tetraazatetracyclo[13.5.2.1^{2,6}.0^{18,21}]tricosa-1(20),2,4, 6(23),15,17,21-heptaen-9-one
8,10-dimethyl-7,14-dioxa-4,10,19,20-tetraazatetracyclo[13.5.2.1^{2,6}.0^{18,21}]tricosa-1(20),2,4, 6(23),15,17,21-heptaene
(13R)-13-methyl-7,14-dioxa-4,10,19,20-tetraazatetracyclo[13.5.2.1^{2,6}.0^{18,21}]tricosa-1(20),2,4,6(23),15(22),16,18(21)-heptaen-9-one
(13R)-13-methyl-7,14-dioxa-4,10,19,20-tetraazatetracyclo[13.5.2.1^{2,6}.0^{18,21}]tricosa-1(20),2,4,6(23),15(22),16,18(21)-heptaene
(7S,9S)-15-oxa-11,20,21-triazapentacyclo[14.5.2.1^{2,6}.0^{7,9}.0^{19,22}]tetracosa-1(21),2(24),3, 5,16(23),17,19(22)-heptaen-10-one
(7S,9S)-15-oxa-11,20,21-triazapentacyclo[14.5.2.1^{2,6}.0^{7,9}.0^{19,22}]tetracosa-1(21),2(24),3,5, 16(23),17,19(22)-heptaene
14-oxa-10,19,20-triazatetracyclo[13.5.2.1^{2,6}.0^{18,21}]tricosa-1(20),2(23),3,5,15(22),16, 18(21)-heptaen-9-one
14-oxa-10,19,20-triazatetracyclo[13.5.2.1^{2,6}.0^{18,21}]tricosa-1(20),2(23),3,5,15(22),16, 18(21)-heptaene
(11S,12S)-11,12-dimethyl-7,14-dioxa-10,19,20-triazatetracyclo[13.5.2.1^{2,6}.0^{18,21}]tricosa-1(20),2(23),3,5,15(22),16,18(21)-heptaen-9-one
(11S,12S)-11,12-dimethyl-7,14-dioxa-10,19,20-triazatetracyclo[13.5.2.1^{2,6}.0^{18,21}]tricosa-1(20),2(23),3,5,15(22),16,18(21)-heptaene
12-methoxy-7,14-dioxa-10,19,20-triazatetracyclo[13.5.2.1^{2,6}.0^{18,21}]tricosa-1(20),2(23),3, 5, 15,17,21-heptaen-9-one
12-methoxy-7,14-dioxa-10,19,20-triazatetracyclo[13.5.2.1^{2,6}.0^{18,21}]tricosa-1(20),2(23),3, 5, 15,17,21-heptaene
(13R)-13-methyl-4-(morpholin-4-yl)-7,14-dioxa-10,19,20-triazatetracyclo[13.5.2.1^{2,6}.0^{18,21}]tricosa-1(20),2(23),3,5,15,17,21-heptaen-9-one
(13R)-13-methyl-4-(morpholin-4-yl)-7,14-dioxa-10,19,20-triazatetracyclo[13.5.2.1^{2,6}.0^{18,21}]tricosa-1(20),2(23),3,5,15,17,21-heptaene
(13S)-13-methyl-4-(morpholin-4-yl)-7,14-dioxa-10,19,20-triazatetracyclo[13.5.2.1^{2,6}.0^{18,21}]tricosa-1(20),2(23),3,5,15,17,21-heptaen-9-one
(13S)-13-methyl-4-(morpholin-4-yl)-7,14-dioxa-10,19,20-triazatetracyclo[13.5.2.1^{2,6}.0^{18,21}]tricosa-1(20),2(23),3,5,15,17,21-heptaene
8-methyl-4-(morpholin-4-yl)-7,14-dioxa-10,19,20-triazatetracyclo[13.5.2.1^{2,6}.0^{18,21}] tricosa-1(20),2,4,6(23),15,17,21-heptaen-9-one
(8R) or (8S)-8-methyl-4-(morpholin-4-yl)-7,14-dioxa-10,19,20-triazatetracyclo[13.5.2.1^{2,6}.0^{18,21}]tricosa-1(20),2,4,6(23),15,17,21-heptaene
(8S) or (8R)-8-methyl-4-(morpholin-4-yl)-7,14-dioxa-10,19,20-triazatetracyclo[13.5.2.1^{2,6}.0^{18,21}]tricosa-1(20),2,4,6(23),15,17,21-heptaene
(12R)-12-methyl-13-oxa-4,5,9,18,19-pentaazatetracyclo[12.5.2.1^{2,5}.0^{17,20}]docosa-1(19), 2(22),3,14(21),15,17(20)-hexaen-8-one
(12R)-12-methyl-13-oxa-4,5,9,18,19-pentaazatetracyclo[12.5.2.1^{2,5}.0^{17,20}]docosa-1(19),2(22),3,14(21),15,17(20)-hexaene
13-oxa-4,5,9,18,19,22-hexaazatetracyclo[12.5.2.1^{2,5}.0^{17,20}]docosa-1(19),2(22),3,14(21),15, 17(20)-hexaen-8-one
13-oxa-4,5,9,18,19,22-hexaazatetracyclo[12.5.2.1^{2,5}.0^{17,20}]docosa-1(19),2(22),3,14(21),15, 17(20)-hexaene
(11S,12S)-12-fluoro-11-methyl-7,14-dioxa-10,19,20-triazatetracyclo[13.5.2.1^{2,6}.0^{18,21}] tricosa-1(20),2,4,6(23),15,17,21-heptaen-9-one
(11S,12S)-12-fluoro-11-methyl-7,14-dioxa-10,19,20-triazatetracyclo[13.5.2.1^{2,6}.0^{18,21}] tricosa-1(20),2,4,6(23),15,17,21-heptaene
4-(trifluoromethyl)-7,14-dioxa-10,19,20-triazatetracyclo[13.5.2.1^{2,6}.0^{18,21}]tricosa-1(20),2,4,6(23),15,17,21-heptaen-9-one
4-(trifluoromethyl)-7,14-dioxa-10,19,20-triazatetracyclo[13.5.2.1^{2,6}.0^{18,21}]tricosa-1(20),2,4,6(23),15,17,21-heptaene
16-methyl-7,14-dioxa-4,10,19,20-tetraazatetracyclo[13.5.2.1^{2,6}.0^{18,21}]tricosa-1(20),2(23), 3,5,15(22),16,18(21)-heptaen-9-one
16-methyl-7,14-dioxa-4,10,19,20-tetraazatetracyclo[13.5.2.1^{2,6}.0^{18,21}]tricosa-1(20),2(23), 3,5,15(22),16,18(21)-heptaene
10-methyl-7,14-dioxa-5,10,19,20-tetraazatetracyclo[13.5.2.1^{2,6}.0^{18,21}]tricosa-1(20),2(23), 3,5,15(22),16,18(21)-heptaen-9-one
10-methyl-7-oxa-4,10,14,19,20-pentaazatetracyclo[13.5.2.1^{2,6}.0^{18,21}]tricosa-1(20),2,4, 6(23),15,17,21-heptaen-9-one
10-methyl-9-oxo-7,14-dioxa-10,19,20-triazatetracyclo[13.5.2.1^{2,6}.0^{18,21}]tricosa-1(20),2,4,6(23),15,17,21-heptaene-4-carbonitrile
7,13-dioxa-10,18,19-triazatetracyclo[12.5.2.1^{2,6}.0^{17,20}]docosa-1(19),2(22),3,5,14(21), 15,17(20)-heptaen-9-one
7,14-dioxa-4,10,19,20,23-pentaazatetracyclo[13.5.2.1^{2,6}.0^{18,21}]tricosa-1(20),2(23),3,5, 15(22),16,18(21)-heptaen-9-one
10-methyl-7,14-dioxa-3,10,19,20-tetraazatetracyclo[13.5.2.1^{2,6}.0^{18,21}]tricosa-1(20),2(23),3, 5,15(22),16,18(21)-heptaen-9-one
4-fluoro-7,14-dioxa-10,19,20-triazatetracyclo[13.5.2.1^{2,6}.0^{18,21}]tricosa-1(20),2(23),3,5, 15(22),16,18(21)-heptaen-9-one
7,14-dioxa-5,10,19,20,23-pentaazatetracyclo[13.5.2.1^{2,6}.0^{18,21}]tricosa-1(20),2(23),3, 5, 15(22),16,18(21)-heptaen-9-one
8,8,10-trimethyl-7,14-dioxa-4,10,19,20-tetraazatetracyclo[13.5.2.1^{2,6}.0^{18,21}]tricosa-1(20),2(23),3,5,15(22),16,18(21)-heptaen-9-one
11-methyl-7,15-dioxa-4,11,20,21-tetraazatetracyclo[14.5.2.1^{2,6}.0^{19,22}]tetracosa-1(21),2,4, 6(24),16,18,22-heptaen-10-one
7,13-dioxa-4,10,18,19-tetraazatetracyclo[12.5.2.1^{2,6}.0^{17,20}]docosa-1(19),2(22),3,5,14(21), 15,17(20)-heptaen-9-one
3-fluoro-10-methyl-7,14-dioxa-4,10,19,20-tetraazatetracyclo[13.5.2.1^{2,6}.0^{18,21}]tricosa-1(20),2,4,6(23),15(22),16,18(21)-heptaen-9-one
8,8-dimethyl-7,14-dioxa-4,10,19,20-tetraazatetracyclo[13.5.2.1^{2,6}.0^{18,21}]tricosa-1(20),2(23),3, 5,15,17,21-heptaen-9-one
12,12-difluoro-7,14-dioxa-4,10,19,20-tetraazatetracyclo[13.5.2.1^{2,6}.0^{18,21}]tricosa-1(20),2,4,6(23),15,17,21-heptaen-9-one
4-bromo-7,14-dioxa-10,19,20-triazatetracyclo[13.5.2.1^{2,6}.0^{18,21}]tricosa-1(20),2(23),3, 5,15,17,21-heptaen-9-one
4-(piperidin-1-yl)-7,14-dioxa-10,19,20-triazatetracyclo[13.5.2.1^{2,6}.0^{18,21}]tricosa-1(20),2(23),3, 5,15,17,21-heptaen-9-one
4-(pyrrolidin-1-yl)-7,14-dioxa-10,19,20-triazatetracyclo[13.5.2.1^{2,6}.0^{18,21}]tricosa-1(20),2(23),3, 5,15,17,21-heptaen-9-one
4-(3,3-difluoropyrrolidin-1-yl)-7,14-dioxa-10,19,20-triazatetracyclo[13.5.2.1^{2,6}.0^{18,21}] tricosa-1(20),2(23),3,5,15,17,21-heptaen-9-one
4-(morpholin-4-yl)-7,14-dioxa-10,19,20-triazatetracyclo[13.5.2.1^{2,6}.0^{18,21}]tricosa-1(20),2(23),3,5,15,17,21-heptaen-9-one
4-(3,6-dihydro-2H-pyran-4-yl)-7,14-dioxa-10,19,20-triazatetracyclo[13.5.2.1^{2,6}.0^{18,21}] tricosa-1(20),2(23),3,5,15,17,21-heptaen-9-one
4-[4-(cyclopropylmethyl)piperazin-1-yl]-7,14-dioxa-10,19,20-triazatetracyclo[13.5.2.1^{2,6}.0^{18,21}]tricosa-1(20),2(23),3,5,15,17,21-heptaen-9-one
4-(5,6-dihydro-2H-pyran-3-yl)-7,14-dioxa-10,19,20-triazatetracyclo[13.5.2.1^{2,6}.0^{18,21}] tricosa-1(20),2(23),3,5,15,17,21-heptaen-9-one
4-(2,5-dihydrofuran-3-yl)-7,14-dioxa-10,19,20-triazatetracyclo[13.5.2.1^{2,6}.0^{18,21}]tricosa-1(20),2(23),3,5,15,17,21-heptaen-9-one
4-(piperazin-1-yl)-7,14-dioxa-10,19,20-triazatetracyclo[13.5.2.1^{2,6}.0^{18,21}]tricosa-1(20),2(23),3,5,15,17,21-heptaen-9-one
4-(furan-3-yl)-7,14-dioxa-10,19,20-triazatetracyclo[13.5.2.1^{2,6}.0^{18,21}]tricosa-1(20),2(23),3, 5,15,17,21-heptaen-9-one
4-(oxolan-3-yl)-7,14-dioxa-10,19,20-triazatetracyclo[13.5.2.1^{2,6}.0^{18,21}]tricosa-1(20),2(23),3, 5,15,17,21-heptaen-9-one
(11R,12R)-11,12-dimethyl-7,14-dioxa-10,19,20-triazatetracyclo[13.5.2.1^{2,6}.0^{18,21}]tricosa-1(20),2,4,6(23),15(22),16,18(21)-heptaen-9-one
4-[(3R,4R)-3,4-difluoropyrrolidin-1-yl]-7,14-dioxa-10,19,20-triazatetracyclo[13.5.2.1^{2,6}.0^{18,21}]tricosa-1(20),2(23),3,5,15,17,21-heptaen-9-one
4-(4-cyclopropylpiperazin-1-yl)-7,14-dioxa-10,19,20-triazatetracyclo[13.5.2.1^{2,6}.0^{18,21}] tricosa-1(20),2(23),3,5,15,17,21-heptaen-9-one
4-(oxan-2-yl)-7,14-dioxa-10,19,20-triazatetracyclo[13.5.2.1^{2,6}.0^{18,21}]tricosa-1(20),2(23),3, 5,15,17,21-heptaen-9-one
4-fluoro-14-oxa-10,19,20-triazatetracyclo[13.5.2.1^{2,6}.0^{18,21}]tricosa-1(20),2(23),3,5,15(22), 16,18(21)-heptaen-9-one
14-oxa-4,10,19,20-tetraazatetracyclo[13.5.2.1^{2,6}.0^{18,21}]tricosa-1(20),2(23),3,5,15(22), 16,18(21)-heptaen-9-one
11-methyl-7,14-dioxa-11,19,20-triazatetracyclo[13.5.2.1^{2,6}.0^{18,21}]tricosa-1(20),2(23),3,5, 15,17,21-heptaen-10-one
8,14-dioxa-4,5,11,19,20,23-hexaazatetracyclo[13.5.2.1^{2,5}.0^{18,21}]tricosa-1(20),2(23),3, 15(22),16,18(21)-hexaen-10-one
(12R)-12-methyl-13-oxa-4,5,9,18,19,22-hexaazatetracyclo[12.5.2.1^{2,5}.0^{17,20}]docosa-1(19),2(22),3,14(21),15,17(20)-hexaen-8-one
(12R)-12-methyl-13-oxa-4,5,9,18,19,22-hexaazatetracyclo[12.5.2.1^{2,5}.0^{17,20}]docosa-1(19),2(22),3,14(21),15,17(20)-hexaen-8-one
(11S)-11-fluoro-13-oxa-4,5,9,18,19,22-hexaazatetracyclo[12.5.2.1^{2,5}.0^{17,20}]docosa-1(19),2(22),3,14(21),15,17(20)-hexaen-8-one
(12S)-12-methyl-13-oxa-4,5,9,18,19,22-hexaazatetracyclo[12.5.2.1^{2,6}.0^{17,20}]docosa-1(19),2(22),3,14(21),15,17(20)-hexaen-8-one
(11R)-11-fluoro-13-oxa-4,5,9,18,19,22-hexaazatetracyclo[12.5.2.1^{2,5}.0^{17,20}]docosa-1(19),2(22),3,14(21),15,17(20)-hexaen-8-one
(11R,12S)-11,12-dimethyl-7,14-dioxa-10,19,20-triazatetracyclo[13.5.2.1^{2,6}.0^{18,21}]tricosa-1(20),2,4,6(23),15(22),16,18(21)-heptaene
(8R or 8S,13 S)-8,13-dimethyl-7,14-dioxa-4,10,19,20-tetraazatetracyclo[13.5.2.1^{2,6}.0^{18,21}] tricosa-1(20),2,4,6(23),15(22),16,18(21)-heptaene
(8S or 8R,13 S)-8,13-dimethyl-7,14-dioxa-4,10,19,20-tetraazatetracyclo[13.5.2.1^{2,6}.0^{18,21}] tricosa-1(20),2,4,6(23),15(22),16,18(21)-heptaene
(11R,12R)-12-fluoro-11-methyl-7,14-dioxa-10,19,20-triazatetracyclo[13.5.2.1^{2,6}.0^{18,21}] tricosa-1(20),2,4,6(23),15(22),16,18(21)-heptaene
(13S)-13-methyl-7,14-dioxa-4,10,19,20-tetraazatetracyclo[13.5.2.1^{2,6}.0^{18,21}]tricosa-1(20),2,4,6(23),15(22),16,18(21)-heptaene
(12S)-12-methyl-13-oxa-4,5,9,18,19-pentaazatetracyclo[12.5.2.1^{2,6}.0^{17,20}]docosa-1(19),2(22),3,14(21),15,17(20)-hexaene
(7S or 7R,13R)-7,13-dimethyl-8,14-dioxa-4,11,19,20-tetraazatetracyclo[13.5.2.1^{2,6}.0^{18,21}] tricosa-1(20),2(23),3,5,15(22),16,18(21)-heptaene
(7R or 7S,13R)-7,13-dimethyl-8,14-dioxa-4,11,19,20-tetraazatetracyclo[13.5.2.1^{2,6}.0^{18,21}] tricosa-1(20),2(23),3,5,15(22),16,18(21)-heptaene
4-(morpholin-4-yl)-7,14-dioxa-11,19,20-triazatetracyclo[13.5.2.1^{2,6}.0^{18,21}]tricosa-1(20),2,4,6(23),15,17,21-heptaene
7,14-dioxa-10,19,20,23-tetraazatetracyclo[13.5.2.1^{2,6}.0^{18,21}]tricosa-1(20),2,4,6(23),15, 17,21-heptaene
7,14-dioxa-4,11,19,20-tetraazatetracyclo[13.5.2.1^{2,6}.0^{18,21}]tricosa-1(20),2,4,6(23),15, 17,21-heptaene
10-methyl-4-(propan-2-yloxy)-7,14-dioxa-10,19,20-triazatetracyclo[13.5.2.1^{2,6}.0^{18,21}] tricosa-1(20),2,4,6(23),15,17,21-heptaene
11-methyl-7,14-dioxa-10,19,20,23-tetraazatetracyclo[13.5.2.1^{2,6}.0^{18,21}]tricosa-1(20),2,4,6(23),15,17,21-heptaene
11,11-difluoro-13-oxa-4,5,9,18,19,22-hexaazatetracyclo[12.5.2.1^{2,5}.0^{17,20}]docosa-1(19),2(22),3,14(21),15,17(20)-hexaen-8-one
(6S,12R)-6,12-dimethyl-18-(oxan-2-yl)-13-oxa-4,5,9,18,19,22-hexaazatetracyclo[12.5.2.1^{2,5}.0^{17,20}]docosa-1(19),2(22),3,14(21),15,17(20)-hexaen-8-one
(6S,12R)-6,9,12-trimethyl-18-(oxan-2-yl)-13-oxa-4,5,9,18,19,22-hexaazatetracyclo [12.5.2.1^{2,5}.0^{17,20}]docosa-1(19),2(22),3,14(21),15,17(20)-hexaen-8-one
(6S,12R)-6,9,12-trimethyl-13-oxa-4,5,9,18,19,22-hexaazatetracyclo[12.5.2.1^{2,5}.0^{17,20}] docosa-1(19),2(22),3,14(21),15,17(20)-hexaene
(12R)-12-methyl-9,13-dioxa-4,5,15,18,19-pentaazatetracyclo[12.5.2.1^{2,5}.0^{17,20}]docosa-1(19),2(22),3,14(21),15,17(20)-hexaene
(12S)-12-methyl-8,11,14-trioxa-4,5,16,19,20-pentaazatetracyclo[13.5.2.1^{2,6}.0^{18,21}]tricosa-1(20),2(23),3,15(22),16,18(21)-hexaene.

Pharmacological studies of the compounds of the invention of formula (I) exhibit inhibitory activity against LRRK2 kinase, including LRRK2 mutant kinase, such as mutant p.G2019S. Kinase activity can be determined using a kinase assay, which typically employs a kinase substrate and a phosphate group donor such as ATP (or a derivative thereof). An exemplary kinase assay is described in the Pharmacological Study.

Compounds of formula (I) of the invention or pharmaceutically acceptable salts thereof are inhibitors of LRRK2 kinase activity and are thus believed to be of potential use in the treatment of or prevention of diseases associated with or characterized by LRRK2 kinase activity such as neurological diseases, endosomal-lysosomal disorders, inflammatory diseases, bacterial, viral and parasitic infections, cardiovascular diseases, autoimmune diseases and cancers.

Particularly compounds of the invention are useful in the treatment of neurological diseases including but not limited to Parkinson's disease (including sporadic Parkinson's disease patients as well as patients with LRRK2 mutations such as p.G2019S or Rab29/Rab7L1 polymorphisms), Alzheimer's disease, amyotrophic lateral sclerosis (ALS), dementia (including Lewy body dementia and vascular dementia, HIV-induced dementia), diabetic neuropathy, age related memory disfunction, mild cognitive impairment, argyrophilic grain disease, Pick's disease, epilepsy, tauopathies such as progressive supranuclear palsy and corticobasal degeneration, synucleinopathies such as multiple system atrophy, frontotemporal dementia, inherited frontotemporal dementia and parkinsonism linked to chromosome 17 (FTDP-17), withdrawal symptoms/relapse associated with drug addiction, L-Dopa induced dyskinesia, ischemic stroke, traumatic brain injury, spinal cord injury and multiple sclerosis.

Other diseases potentially treatable by inhibition of LRRK2 activity are endosomal-lysosomal diseases including but not limited to Niemann-Pick Type A, B or C disease, Gaucher's disease, Krabbe's disease, Fabry's disease and disorders with mitochondrial deficits; inflammatory diseases including but not limited to vasculitis, pulmonary diseases such as chronic obstructive pulmonary disease, idiopathic pulmonary fibrosis, inflammatory myopathies, ankylosing spondylitis; autoimmune diseases including but not limited to Crohn's disease, inflammatory bowel disease, rheumatoid arthritis, ulcerative colitis, lupus, autoimmune hemolytic anemia, pure red cell aplasia, idiopathic thrombocytopenic purpura, type I diabetes mellitus, obesity, Evans syndrome, bullous skin disorders, Sjogren's syndrome, Devic's disease and leprosy. Compounds of the invention have also anti carcinogenic effects and are potentially useful in the treatment of cancers including but not limited to thyroid cancer, renal cancer (including papillary renal), breast cancer, hormone-related cancer, adeno-and squamous lung cancer, non-small-cell lung cancer, colon cancer, prostate cancers, skin cancers, leukemias (including acute myelogenous leukemia) and lymphomas.

Compounds of the invention are also potentially useful in the treatment of cardiovascular diseases including but not limited to stroke.

Other diseases potentially treatable by compounds of the invention are bacterial infections such as but not limited to leprosy and tuberculosis; viral infections such as but not limited to coronavirus such as SARS-CoV, MERS-CoV and SARS-CoV-2, HIV, West Nile virus and chikungunya virus.

Another aspect of the invention is related to pharmaceutical compositions comprising at least one compound of formula (I) in combination with one or more pharmaceutically acceptable excipients. In particular, these pharmaceutical compositions are interesting for use in the treatment or prevention of diseases associated with or characterized by LRRK2 kinase activity such as but not limited to neurological diseases, endosomal-lysosomal disorders, inflammatory diseases, bacterial, viral and parasitic infections, cardiovascular diseases, autoimmune diseases and cancers. In a specific embodiment, pharmaceutical compositions of the invention are useful for the treatment or prevention of Parkinson's disease (including sporadic Parkinson's disease patients as well as patients with LRRK2 mutations or Rab29/Rab7L1 polymorphisms), Alzheimer's disease, amyotrophic lateral sclerosis (ALS), dementia (including Lewy body dementia and vascular dementia, HIV-induced dementia), diabetic neuropathy, age related memory disfunction, mild cognitive impairment, argyrophilic grain disease, Pick's disease, epilepsy, tauopathies such as progressive supranuclear palsy and corticobasal degeneration, other synucleinopathies such as multiple system atrophy, frontotemporal dementia, inherited frontotemporal dementia and parkinsonism linked to chromosome 17 (FTDP-17), withdrawal symptoms/relapse associated with drug addiction, L-Dopa induced dyskinesia, ischemic stroke, traumatic brain injury, spinal cord injury, multiple sclerosis, Niemann-Pick Type A, B or C disease, Gaucher's disease, Krabbe's disease, Fabry's disease, disorders with mitochondrial deficits, Crohn's disease, inflammatory bowel disease, rheumatoid arthritis, ulcerative colitis, lupus, autoimmune hemolytic anemia, pure red cell aplasia, idiopathic thrombocytopenic purpura, type I diabetes mellitus, obesity, Evans syndrome, bullous skin disorders, Sjogren's syndrome, Devic's disease, leprosy, thyroid cancer, renal cancer (including papillary renal), breast cancer, hormone-related cancer, adeno-and squamous lung cancer, non-small-cell lung cancer, colon cancer, prostate cancers, skin cancers, leukemias (including acute myelogenous leukemia), lymphomas, stroke, leprosy, tuberculosis, and SARS-CoV, MERS-CoV, SARS-CoV-2, HIV, West Nile virus and chikungunya virus infections.

Among the pharmaceutical compositions according to the invention there may be mentioned more especially those that are suitable for oral, parenteral, nasal, per- or trans-cutaneous, rectal, perlingual, ocular or respiratory administration, especially tablets or dragées, sublingual tablets, sachets, paquets, capsules, glossettes, lozenges, suppositories, creams, ointments, dermal gels, and drinkable or injectable ampoules.

The pharmaceutical compositions according to the invention comprise one or more excipients or carriers selected from diluents, lubricants, binders, disintegration agents, stabilisers, preservatives, absorbents, colorants, sweeteners, flavourings etc.

By Way of Non-Limiting Example there May be Mentioned:
as diluents: lactose, dextrose, sucrose, mannitol, sorbitol, cellulose, glycerol,
as lubricants: silica, talc, stearic acid and its magnesium and calcium salts, polyethylene glycol,
as binders: magnesium aluminium silicate, starch, gelatin, tragacanth, methylcellulose, sodium carboxymethylcellulose and polyvinylpyrrolidone,
as disintegrants: agar, alginic acid and its sodium salt, effervescent mixtures.

The dosage varies according to the sex, age and weight of the patient, the administration route, the nature of the therapeutic indication, or of any associated treatments, and ranges from 0.01 mg to 1 g per 24 hours in one or more administrations.

The following Preparations and Examples illustrate the invention but do not limit it in any way. The compounds of this invention can be prepared by any of several standard synthetic processes commonly used by those skilled in the art of organic chemistry. The compounds are generally prepared from starting materials which are either commercially available or prepared by standard means obvious to those skilled in the art.

### General Schemes

As indicated herein before, the present invention provides compounds according to formula (I):

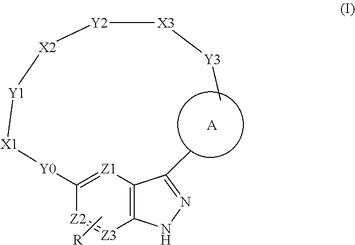

wherein R, Z1, Z2, Z3, Y0, X1, Y1, X2, Y2, X3, Y3 and A are as defined for formula (I). With reference to the general reaction schemes suitable for preparing said compounds, these compounds can be represented by formula (I), for which the general reaction schemes can be found herein below. In the general schemes below, R, Z1, Z2, Z3, Y0, X1, Y1, X2, Y2, X3, Y3, Ra and A will have the same meaning as defined for formula (I).

The fused pyrazolo bicyclic structure containing Z1, Z2, Z3 and R will be referred to as fused pyrazolo structure in the followings.

In the general schemes below, Lg1, Lg2, Lg3, and Lg4, each independently represent suitable leaving groups. Pg1 represents a protective group suitable to protect the NH of the fused pyrazolo structure. Pg2 and Pg3 each independently represent a suitable protecting group that can be used to protect X1 and/or X2.

Rb in the schemes below can be either H, alkyl or a cyclic alkyl.

In all of the general schemes below, before deprotection of the NH of the fused pyrazolo structure, an optional cross-coupling reaction such as a Buchwald, Suzuki, Sonogashira reaction or alternatively an O-alkylation or nucleophilic aromatic substitution can be carried out on the (hetero-) aromatic ring which contains a leaving group such as a halide, to form a compound of formula (XVI). After the cross-coupling reaction such as a Buchwald, Suzuki, Sonogashira reaction or alternatively an O-alkylation or nucleophilic aromatic substitution, the NH of the fused pyrazolo structure can be deprotected to result in the final compound of formula (I).

### Scheme A

The compounds of formula (I), can be prepared as shown in general Scheme A below wherein the 1H-fused pyrazolo structure of formula (II) is alkylated with a compound of formula (III) containing a functional group FgA, which can be transformed during the synthesis route into a moiety suitable for cyclization such as for example mesylate or tosylate. After alkylation, the compound of formula (IV) is coupled in a cross-coupling reaction such as a Suzuki reaction with a compound of formula (V) to yield a compound of formula (VI). The resulting compound of formula (VI) can be macrocyclized by an etherification reaction such as a Williamson or Mitsunobu reaction affording a compound of formula (VII). Final deprotection of the fused pyrazolo structure nitrogen, either or not after substitution of the A ring results in the compound of formula (I).

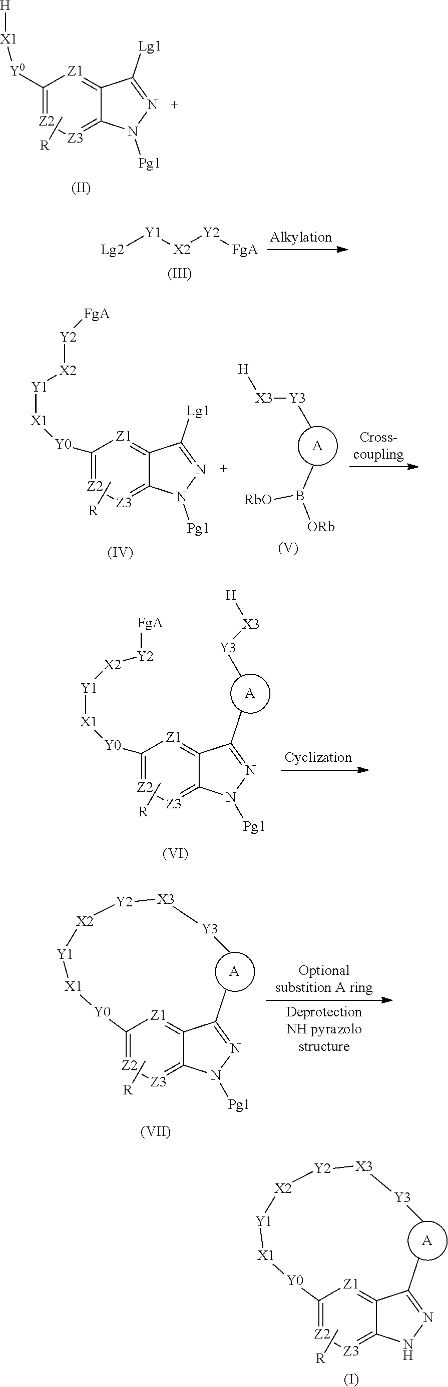

### In the Above Scheme A

The alkylation between a compound of formula (II) with a compound of formula (III) can be accomplished in a solvent such as DMF or DMA and a base such as cesium carbonate at RT or at an elevated temperature such as 60° C. or 100° C.

Organometallic cross coupling such as Suzuki coupling of the compound of formula (IV) with a compound of formula (V) can be done using palladium catalysts such as for example tetrakis(triphenylphosphine)palladium(0) combined or not with 2-dicyclohexylphosphino-2′,4′,6′-triisopropylbiphenyl (Xphos) in the presence of potassium phosphate tribasic or sodium carbonate in a solvent mixture such as for example dioxane/water or DME/water at an elevated temperature such as for example 80° C. or 90° C.

The cyclisation of the compound of formula (VI) to give compound of formula (VII) can be performed by an etherification reaction such as a Williamson reaction using cesium carbonate in a solvent such as dry DMF or DMA at for example an elevated temperature of 80° C. or 90° C.

Alternatively, the compound of formula (VI) can be cyclized under Mitsunobu conditions using for example DIAD and Ph₃P in a solvent mixture such as MeTHF/toluene at an elevated temperature such as 90° C.

Final deprotection of the fused pyrazolo structure NH in the compound of formula (VII) can be achieved under acidic conditions such as 4N HCl in dioxane or TFA in DCM at room temperature, either or not after substitution of the A ring to yield the final compound of formula (I).

### Scheme B

Alternatively, the compounds of formula (I) can be prepared as shown in general Scheme B below wherein the boronate of formula (VIII) is alkylated with a compound of formula (IX) containing a functional group FgA, which can be transformed during the synthesis route into a moiety suitable for cyclization such as for example mesylate or tosylate. After alkylation, the compound of formula (X) is coupled in a cross-coupling reaction such as a Suzuki reaction with the fused pyrazolo structure of formula (II) to yield a compound of formula (XI). The resulting compound of formula (XI) can be macrocyclized by an etherification reaction such as a Williamson or Mitsunobu reaction affording a compound of formula (VII). Final deprotection of the fused pyrazolo structure nitrogen, either or not after substitution of the A ring results in the compound of formula (I).

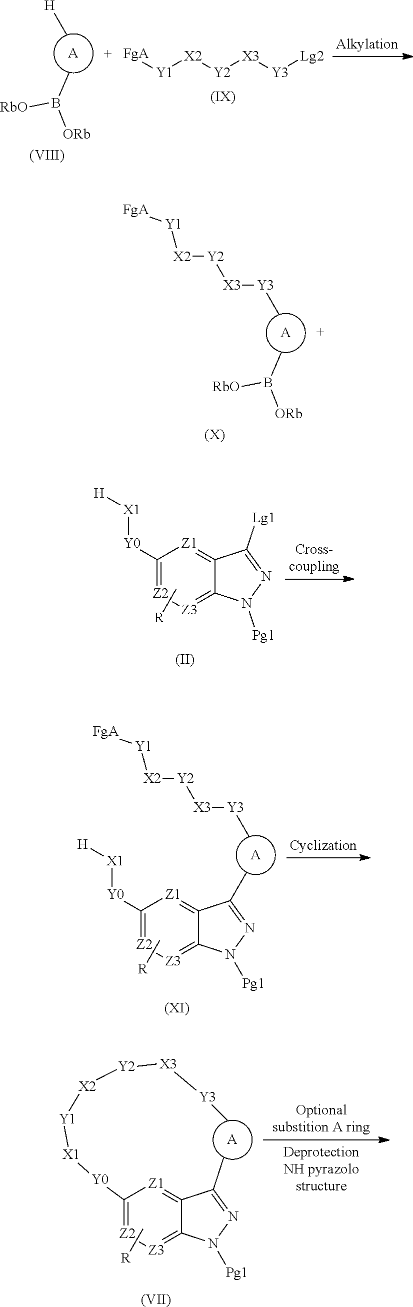

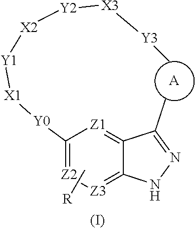

### In the Above Scheme B

The alkylation between a compound of formula (VIII) with a compound of formula (IX) can be accomplished using for example sodium hydride in a dry solvent such as dry DMF at a temperature ranging from 0° C. to RT.

Organometallic cross coupling such as Suzuki coupling of the compound of formula (X) with a compound of formula (II) can be done using palladium catalysts such as for example tetrakis(triphenylphosphine)palladium(0) combined or not with 2-dicyclohexylphosphino-2′,4′,6′-triisopropylbiphenyl (Xphos) in the presence of a base such as potassium phosphate tribasic or sodium carbonate in a solvent mixture such as for example dioxane/water or DME/water at an elevated temperature such as for example 80° C. or 90° C.

The cyclisation of the compound of formula (XI) to give compound of formula (VII) can be performed by an etherification reaction such as a Williamson reaction using for example cesium carbonate in a solvent such as dry DMF, DMA or ACN at for example an elevated temperature of 50° C., 80° C. or 90° C. Alternatively, the compound of formula (VI) can be cyclized under Mitsunobu conditions using for example DIAD and Ph₃P in a solvent mixture such as MeTHF/toluene at an elevated temperature such as 90° C.

Final deprotection of the fused pyrazolo structure NH in the compound of formula (VII) can be achieved under acidic conditions such as 4N HCl in dioxane or TFA in DCM or pTSA in a solvent mixture such as MeOH/H₂O at room temperature, either or not after substitution of the A ring to yield the final compound of formula (I).

### Scheme C

Alternatively, the compounds of formula (I) can be prepared as shown in general Scheme C below wherein the boronate of formula (XII) is coupled in a cross-coupling reaction with a group of formula (XIII). After cross-coupling the compound of formula (XIV) is alkylated with a compound of formula (XV) containing a functional group FgA, which can be transformed during the synthesis route into a moiety suitable for cyclization such as for example mesylate or tosylate. After alkylation, the compound of formula (XVI) is deprotected to yield a compound of formula (XI). The resulting compound of formula (XI) can be macrocyclized by an etherification reaction such as a Williamson or Mitsunobu reaction affording a compound of formula (VII). Final deprotection of the fused pyrazolo structure nitrogen, either or not after substitution of the A ring results in the compound of formula (I).

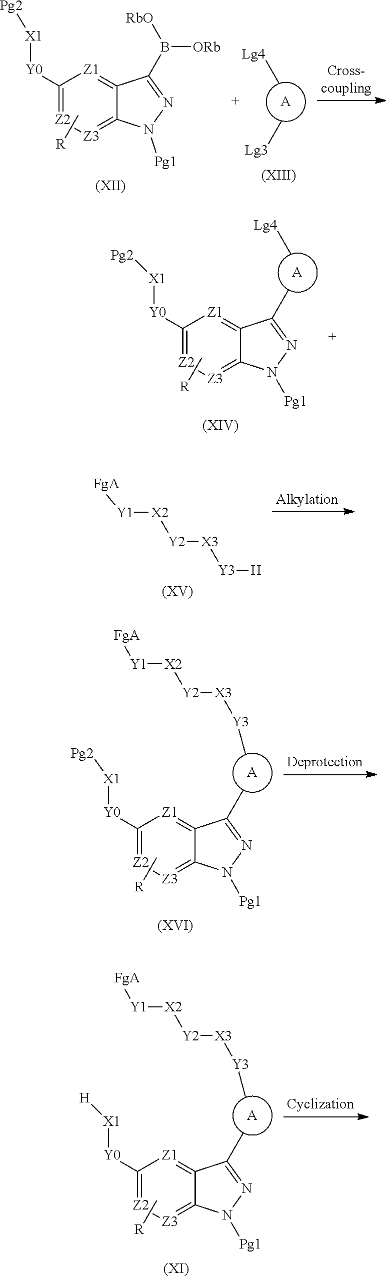

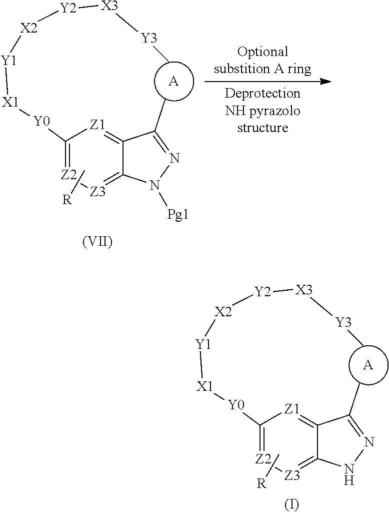

### In the Above Scheme C

Organometallic cross coupling such as Suzuki coupling of the compound of formula (XII) with a compound of formula (XIII) can be done using palladium catalysts such as for example tetrakis(triphenylphosphine)palladium(0) combined or not with 2-dicyclohexylphosphino-2′,4′,6′-triisopropylbiphenyl (Xphos) in the presence of a base such as for example potassium phosphate tribasic or sodium carbonate in a solvent mixture such as for example dioxane/water or DME/water at an elevated temperature such as for example 80° C. or 90° C.

The alkylation between a compound of formula (XIV) with a compound of formula (XV) can be accomplished using for example sodium hydride in a dry solvent such as dry THF at a temperature ranging from 0° C. to RT.

Deprotection of the compound of formula (XVI) can be done using for example TBAF in THF at a temperature ranging from 0° C. to RT.

The cyclisation of the compound of formula (XI) to give compound of formula (VII) can be performed by an etherification reaction such as a Williamson reaction using a base such as for example cesium carbonate in a solvent such as dry DMF, DMA or ACN at for example an elevated temperature of 50° C., 80° C. or 90° C. Alternatively, the compound of formula (VI) can be cyclized under Mitsunobu conditions using for example DIAD and Ph₃P in a solvent mixture such as MeTHF/toluene at an elevated temperature such as 90° C.

Final deprotection of the fused pyrazolo structure NH in the compound of formula (VII) can be achieved under acidic conditions such as 4N HCl in dioxane or TFA in DCM or pTSA in a solvent mixture such as MeOH/H₂O at room temperature, either or not after substitution of the A ring to yield the final compound of formula (I).

### Scheme D

Alternatively, the compounds of formula (I) can be prepared as shown in general Scheme D below wherein the compound of formula (XVII) is alkylated with a compound of formula (IX) containing a functional group FgA, which can be transformed during the synthesis route into a moiety suitable for cyclization such as for example mesylate or tosylate. The resulting compound of formula (XVIII) is coupled in a cross-coupling reaction with a compound of formula (XII). After cross-coupling the compound of formula (XVI) can be optionally deprotected into a compound of formula (XI). The compound of formula (XVI) can be macrocyclized by a one-pot etherification reaction such as a Williamson affording a compound of formula (VII). Alternatively, the compound of formula (XI) can be macrocyclized by an etherification reaction such as a Williamson or Mitsunobu reaction affording a compound of formula (VII). Final deprotection of the fused pyrazolo structure nitrogen, either or not after substitution of the A ring results in the compound of formula (I).

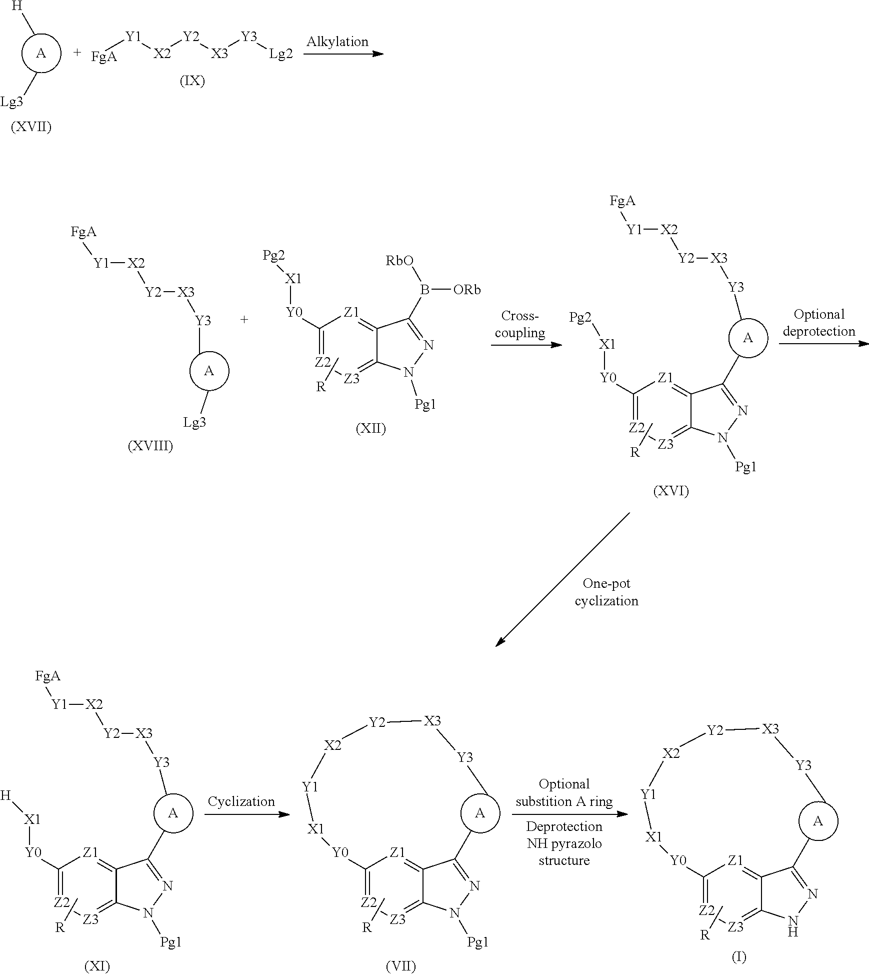

### In the Above Scheme D

The alkylation between a compound of formula (XVII) with a compound of formula (IX) can be accomplished using for example sodium hydride in a dry solvent such as dry DMF at a temperature ranging from 0° C. to RT.

Organometallic cross coupling such as Suzuki coupling of the compound of formula (XVIII) with a compound of formula (XII) can be done using palladium catalysts such as for example tetrakis(triphenylphosphine)palladium(0) combined or not with 2-dicyclohexylphosphino-2′,4′,6′-triisopropylbiphenyl (Xphos) in the presence of a base such as for example potassium phosphate tribasic or sodium carbonate in a solvent mixture such as for example dioxane/water or DME/water at an elevated temperature such as for example 80° C. or 90° C.

Optional deprotection of the compound of formula (XVI) can be done using for example TBAF in THF at a temperature ranging from 0° C. to RT.

The cyclisation of the compound of formula (XVI) or formula (XI) to give compound of formula (VII) can be performed by an etherification reaction such as a Williamson reaction using a base such as for example cesium carbonate in a solvent such as dry DMF, DMA or ACN at for example an elevated temperature of 50° C., 80° C. or 90° C. Alternatively, the compound of formula (XI) can be cyclized under Mitsunobu conditions using for example DIAD and Ph₃P in a solvent mixture such as MeTHF/toluene at an elevated temperature such as 90° C.

Final deprotection of the fused pyrazolo structure NH in the compound of formula (VII) can be achieved under acidic conditions such as 4N HCl in dioxane or TFA in DCM or pTSA in a solvent mixture such as MeOH/H₂O at room temperature, either or not after substitution of the A ring to yield the final compound of formula (I).

### Scheme E

Alternatively, the compounds of formula (I) can be prepared as shown in general Scheme E below wherein the compound of formula (XII) is coupled in a cross-coupling reaction with a compound of formula (XIX) containing a functional group FgB, which can be transformed during the synthesis route into a moiety suitable for cyclization such as for example mesylate or tosylate. The compound of formula (XXI) is alkylated with a compound of formula (XXII).

The compound of formula (XXIII) can be optionally deprotected into a compound of formula (XXIV). The compounds of formula (XXIII) or (XXIV) can be macrocyclized by an etherification reaction such as a Williamson reaction affording a compound of formula (VII).

Final deprotection of the fused pyrazolo structure nitrogen, either or not after substitution of the A ring results in the compound of formula (I).

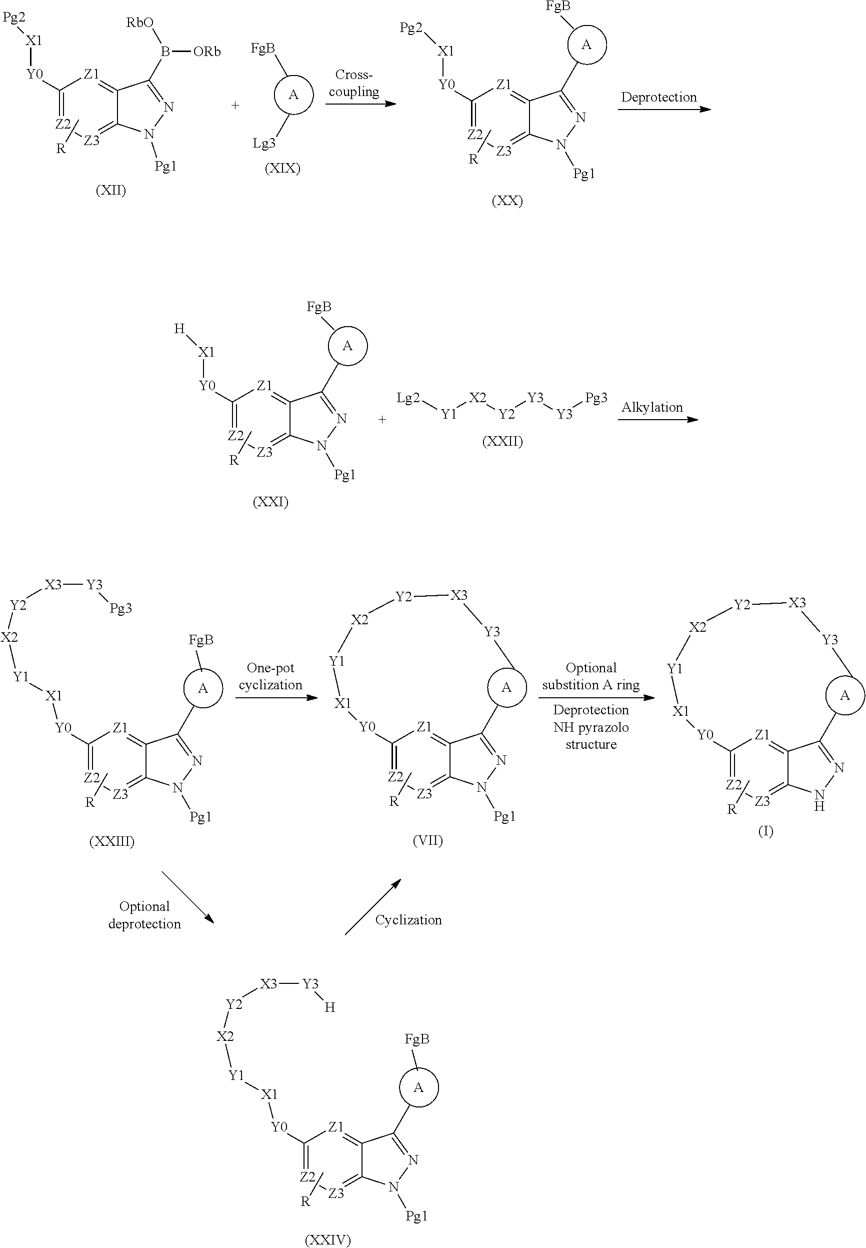

### In the Above Scheme E

Organometallic cross coupling such as Suzuki coupling of the compound of formula (XII) with a compound of formula (XIX) can be done using palladium catalysts such as for example tetrakis(triphenylphosphine)palladium(0) combined or not with 2-dicyclohexylphosphino-2′,4′,6′-triisopropylbiphenyl (Xphos) in the presence of potassium phosphate tribasic or sodium carbonate in a solvent mixture such as for example dioxane/water or DME/water at an elevated temperature such as for example 80° C. or 90° C.

Deprotection of the compound of formula (XX) can be done using for example TBAF in THF at a temperature ranging from 0° C. to RT.

The alkylation between a compound of formula (XXI) with a compound of formula (XXII) can be accomplished using sodium hydride in a dry solvent such as dry THF at a temperature ranging from 0° C. to RT or can be accomplished using a base such as cesium carbonate in DMF at RT. Alternatively, the alkylation can be done using Mitsunobu conditions using for example DIAD and Ph₃P in a solvent such as THF at RT or at an elevated temperature such as 90° C.

Optional deprotection of the compound of formula (XXIII) can be done using TBAF in THF at a temperature ranging from 0° C. to RT.

The cyclisation of the compound of formula (XXIII) or formula (XXIV) to give compound of formula (VII) can be performed by an etherification reaction such as a Williamson reaction using a base such as for example cesium carbonate in a solvent such as dry DMF, DMA or ACN at for example an elevated temperature of 50° C., 80° C. or 90° C. The cyclisation of the compound of formula (XXIV) can also be done using sodium hydride in dry THF at an elevated temperature such as 60° C.

Final deprotection of the fused pyrazolo structure NH in the compound of formula (VII) can be achieved under acidic conditions such as 4N HCl in dioxane or TFA in DCM or pTSA in a solvent mixture such as MeOH/H₂O at room temperature, either or not after substitution of the A ring to yield the final compound of formula (I).

### Scheme F

Alternatively, the compounds of formula (I), can be prepared as shown in general Scheme F below wherein the 1H-fused pyrazolo structure of formula (II) is coupled in a cross-coupling reaction such as a Suzuki reaction with a protected compound of formula (XXV) to yield a compound of formula (XXVI). The compound of formula (XXVI) is alkylated with a compound of formula (XXVII) containing a functional group FgA, which can be transformed during the synthesis route into a moiety suitable for cyclization such as for example mesylate or tosylate. The compound of formula (XXVIII) can be deprotected to yield a compound of formula (XXIX). The resulting compound of formula (XXIX) can be macrocyclized by an etherification reaction such as a Williamson reaction affording a compound of formula (VII). Final deprotection of the fused pyrazolo structure nitrogen, either or not after substitution of the A ring results in the compound of formula (I).

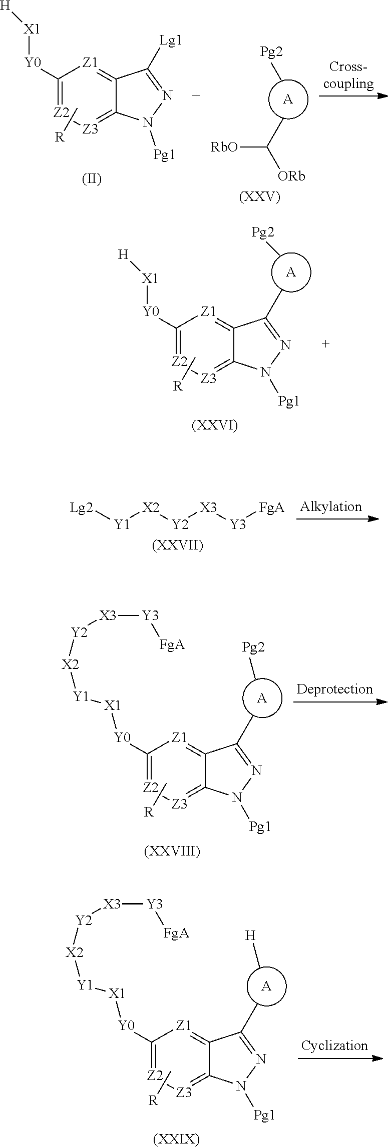

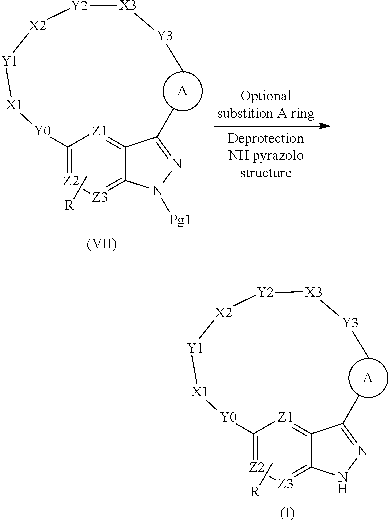

### In the Above Scheme F

Organometallic cross coupling such as Suzuki coupling of the protected compound of formula (XXV) with a compound of formula (II) can be done using palladium catalysts such as for example tetrakis(triphenylphosphine)palladium(0) combined or not with 2-dicyclohexylphosphino-2′,4′,6′-triisopropylbiphenyl (Xphos) in the presence of a base such as for example potassium phosphate tribasic or sodium carbonate in a solvent mixture such as for example dioxane/water or DME/water at an elevated temperature such as for example 100° C. The alkylation between a compound of formula (XXVI) with a compound of formula (XXVII) can be accomplished in a solvent such as DMF or DMA and a base such as cesium carbonate at RT or at an elevated temperature such as 70° C.

Deprotection of the compound of formula (XXVIII) can be achieved using TBAF in a solvent such as THF at an elevated temperature such as 60° C.

The cyclisation of the compound of formula (XXIX) to give compound of formula (VII) can be performed by an etherification reaction such as a Williamson reaction using a base such as for example cesium carbonate in a solvent such as dry DMF or DMA at for example an elevated temperature of 80° C. or 90° C.

Final deprotection of the fused pyrazolo structure NH in the compound of formula (VII) can be achieved under acidic conditions such as 4N HCl in dioxane or TFA in DCM at room temperature, either or not after substitution of the A ring to yield the final compound of formula MI.

### Scheme G

The compounds of formula (I), can be prepared as shown in general Scheme G below wherein the 1H-fused pyrazolo structure of formula (XXX) is alkylated with a compound of formula (XXII). After alkylation, the compound of formula (XXXI) is boronated to a compound of formula (XXXII) and then coupled in a cross-coupling reaction such as a Suzuki reaction with a compound of formula (XIX) containing a functional group FgB, which can be transformed during the synthesis route into a moiety suitable for cyclization such as for example mesylate or tosylate. The resulting compound of formula (XXIII) is deprotected to a compound of formula (XXIV) which can be macrocyclized affording a compound of formula (VII). Final deprotection of the fused pyrazolo structure nitrogen, either or not after substitution of the A ring results in the compound of formula (I).

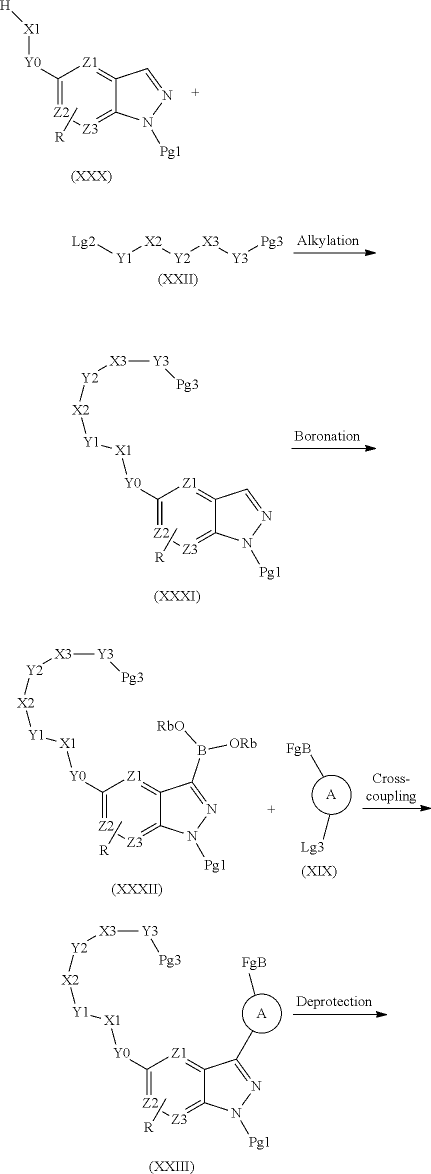

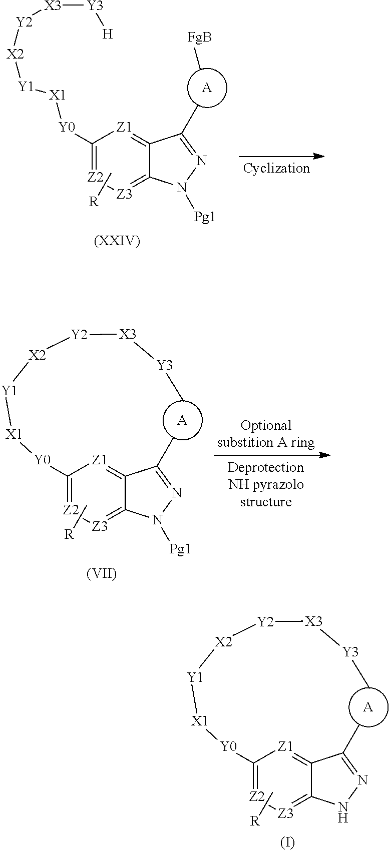

### In the Above Scheme G

The alkylation between a compound of formula (XXX) with a compound of formula (XXII) can be accomplished through a Mitsunobu reaction using for example DIAD and PPh₃ in a solvent such as dry THF at an elevated temperature such as 90° C.

The boronation of the compound of formula (XXXI) can be achieved using bis(pinacolato)diboron, 4,4′-di-tert-butyl-2,2′-bipyridine and 1,5-cyclooctadiene)(methoxy) iridium(I) dimer in a solvent such as dry MTBE at an elevated temperature such as 90° C.

Organometallic cross coupling such as Suzuki coupling of the compound of formula (XXXII) with a compound of formula (XIX) can be done using palladium catalysts such as for example tetrakis(triphenylphosphine)palladium(0) combined or not with 2-dicyclohexylphosphino-2′,4′,6′-triisopropylbiphenyl (Xphos) in the presence of a base such as for example potassium phosphate tribasic or sodium carbonate in a solvent mixture such as for example dioxane/water or DME/water at an elevated temperature such as for example 80° C. or 90° C.

Deprotection of the compound of formula (XXIII) can be done using for example TBAF in THF at a temperature ranging from 0° C. to RT.

The cyclisation of the compound of formula (XXIV) to give compound of formula (VII) can be performed by using for example a lithium bis(trimethylsilyl)amide solution in a solvent such as dry THF at RT.

Final deprotection of the fused pyrazolo structure NH in the compound of formula (VII) can be achieved under acidic conditions such as 4N HCl in dioxane or TFA in DCM at room temperature, either or not after substitution of the A ring to yield the compound of formula (I).

### Scheme H

The compounds of formula (I/a), a particular case of compound of formula (I) wherein —Y0- represents a —CH₂— group, can be prepared as shown in general Scheme H below wherein the 1H-fused pyrazolo structure of formula (XXXIII) is coupled in a cross-coupling reaction such as a Suzuki reaction with a compound of formula (V) and then alkylated with a compound of formula (IX). The resulting compound of formula (XXXV) is deprotected to a compound of formula (XXXVI) which can be macrocyclized affording a compound of formula (XXXVII). Reduction and final deprotection of the fused pyrazolo structure nitrogen, either or not after substitution of the A ring results in the compound of formula (I/a).

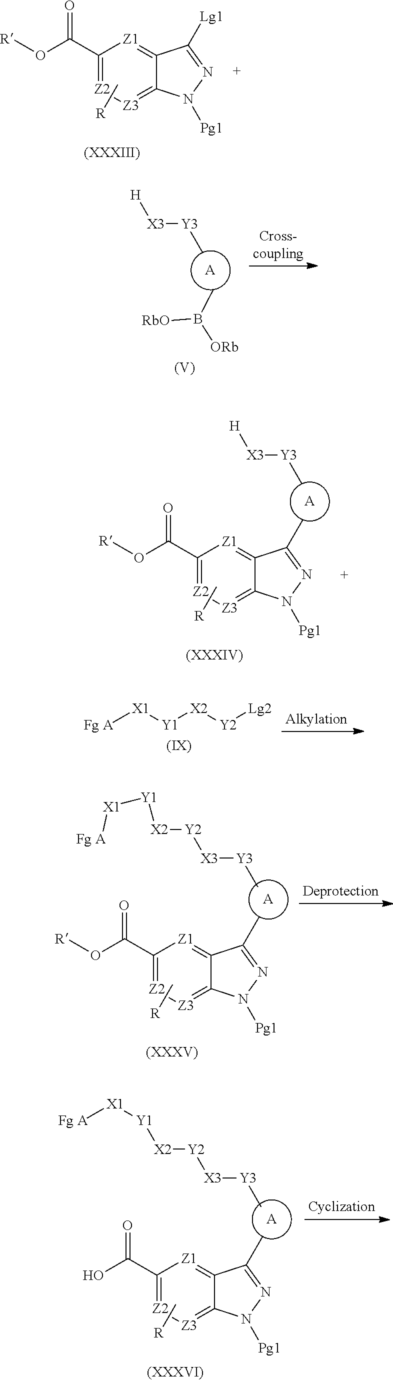

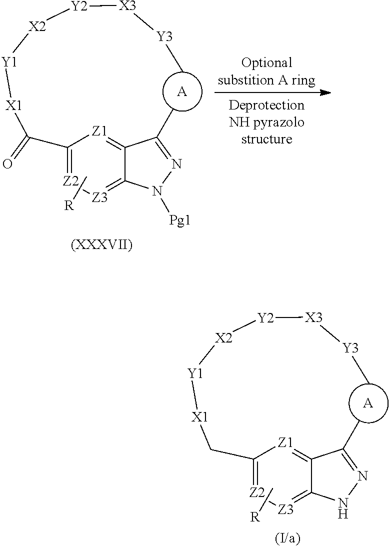

### In the Above Scheme H

Compound of formula (I/a) is a particular case of compound of formula (I) wherein —Y0- represents a —CH₂— group.

Organometallic cross coupling such as Suzuki coupling of the compound of formula (XXXIII) with a compound of formula (V) can be done using palladium catalysts such as for example tetrakis(triphenylphosphine)palladium(0) combined or not with 2-dicyclohexylphosphino-2′,4′,6′-triisopropylbiphenyl (Xphos) in the presence of a base such as for example potassium phosphate tribasic or sodium carbonate in a solvent mixture such as for example dioxane/water or DME/water at an elevated temperature such as for example 110° C.

The alkylation between a compound of formula (XXXIV) with a compound of formula (IX) can be accomplished through a nucleophilic substitution using a base such as for example cesium carbonate in a solvent such as DMF at an elevated temperature such as 80° C.

Deprotection of the compound of formula (XXXV) can be done using for example LiOH in a mixture of THF and water at an elevated temperature such as 60° C.

The cyclisation of the compound of formula (XXXVI) to give compound of formula (XXXVII) can be performed by using for example HBTU and DIPEA in a solvent such as DMA at RT.

Reduction of the carbonyl group of the compound of formula (XXXVII) can be achieved using for example titanium tetrachloride and lithium aluminum hydride in a solvent such as dioxane at a temperature such as RT.

The fused pyrazolo structure NPg1 in the compound of formula (XXXVII) can be deprotected during the reduction step or during subsequent deprotection step.

### Scheme I

The compounds of formula (I/b), a particular case of compound of formula (I) wherein —Y2- represents a —CH₂—Y′2- group, or (I/b′), a particular case of compound of formula (I) wherein —Y2- represents a —C(O)—Y′2- group, can be prepared as shown in general Scheme I below wherein the 1H-fused pyrazolo structure of formula (II) is alkylated with a compound of formula (XXXVIII) containing a leaving group Lg2 such as for example mesylate or bromide. After alkylation, the compound of formula (XXXIX) is coupled in a cross-coupling reaction such as a Suzuki reaction with a compound of formula (V) to yield a compound of formula (XL). The resulting compound of formula (XL) can be alkylated to form a compound of formula (XLI). Alternatively, the compound of formula (XXXIX) can be coupled in a cross-coupling reaction such as a Suzuki reaction with a compound of formula (XLII) to yield a compound of formula (XLI). Deprotection of the compound of formula (XLI) results in a compound of formula (XLIII) or a compound of formula (XLIV). The compound of formula (XLIII) or (XLIV) can be macrocyclized by an amidation reaction, the A ring can optionally be substituted and/or the amide can be reduced to the amine resulting in the compounds of formula (I/b) or (VII′). Eventually, final deprotection of the compound of formula (VII′) gives the compound of formula (I/b′), that can be reduced to amine resulting in the compound of formula (I/b).

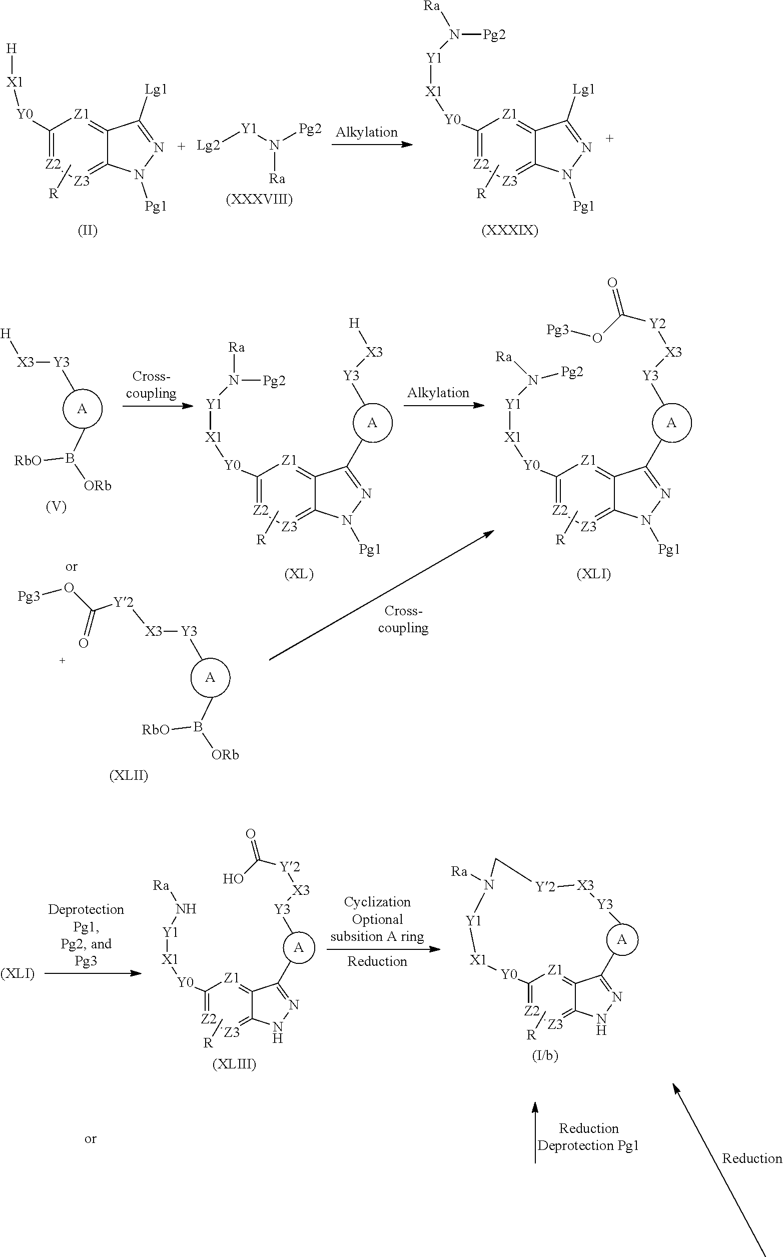

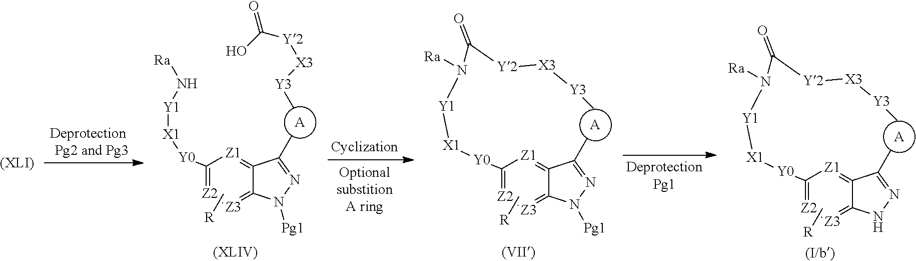

### In the Above Scheme I

Compounds of formula (I/b) and (I/b′) are a particular case of compound of formula (I) wherein —Y2- represents a —CH₂—Y′2- group and a —C(O)—Y′2- group respectively, wherein —Y′2- represents a linear or branched (C₁-C₅) alkanediyl group, a linear or branched (C₂-C₅) alkenediyl group, or a linear or branched (C₃-C₅) cycloalkanediyl group, those groups being optionally substituted as defined for —Y2-.

The alkylation between a compound of formula (II) with a compound of formula (XXXVIII) can be accomplished in a solvent such as DMF or DMA and a base such as cesium carbonate at RT or at an elevated temperature such as 50° C. or 85° C.

Organometallic cross coupling such as Suzuki coupling of the compound of formula (XXXIX) with a compound of formula (V) or a compound of formula (XLII) can be done using palladium catalysts such as for example tetrakis(triphenylphosphine)palladium(O) combined or not with 2-dicyclohexylphosphino-2′,4′,6′-triisopropylbiphenyl (Xphos) in the presence of potassium phosphate tribasic or sodium carbonate in a solvent mixture such as for example dioxane/water or DME/water at an elevated temperature such as for example 110° C. or at 100° C. under microwave conditions.

The alkylation of a compound of formula (XL) can be accomplished using for example a bromide in a solvent such as DMF, DMA or ACN and a base such as cesium carbonate at RT or at an elevated temperature such as 60° C. or 100° C.

Deprotection of the compound of formula (XLI) can be achieved under acidic conditions such as 4N HCl in dioxane or TFA in DCM at room temperature. Alternatively, Pg2 and Pg3 can be removed under hydrogenation conditions using hydrogen gas, Pd/C in a solvent such as ethyl acetate at a temperature such as RT.

The cyclisation of the compound of formula (XLIII) or (XLIV) can be performed by an amidation reaction using N-(dimethylamino)-1H-1,2,3-triazolo-4,5-bpyridin-1-ylmethylene-N-methylmethan aminium hexafluorophosphate N-oxide (HATU) or (benzotriazol-1-yloxy)tripyrrolidino phosphonium hexafluorophosphate (PyBOP) or N,N,N′,N-Tetramethyl-O-(1H-benzotriazol-1-yl)uronium hexafluorophosphate (HBTU), a base such as N,N-diisopropylethylamine (DIPEA) or trimethylamine in a solvent such as DMF or DMA at for example RT.

Reduction of the amide, either or not after substitution of the A ring, can be achieved using borane dimethyl sulfide complex 2N solution in THF at a temperature such as RT or can be done using lithium aluminium hydride (1.0 M solution in THF) and trimethylsilyl chloride in a solvent such as THF at a temperature such as 0° C. and/or RT to yield the compound of formula (I/b).

The deprotection of Pg1 group can be achieved under acidic conditions such as 4N HCl in dioxane or TFA in DCM at room temperature or PTSA in a solvent mixture such as methanol/water at an elevated temperature such as 65° C. to yield the compound of formula (I/b) or (I/b′).

### Scheme J

The compounds of formula (I/c), a particular case of compound of formula (I) wherein —X2- represents —NRa—, can be prepared as shown in general Scheme J below wherein the 1H-fused pyrazolo structure of formula (II) is alkylated with a compound of formula (XXXVIII′) containing a leaving group Lg2 such as for example mesylate or bromide. After alkylation, the compound of formula (XXXIX′) is coupled in a cross-coupling reaction such as a Suzuki reaction with a compound of formula (V′), containing a functional group FgA, which can be transformed during the synthesis route into a moiety suitable for cyclization such as for example mesylate, tosylate or iodine to yield a compound of formula (XLV).

Alternatively, the compound of formula (XXX) can be alkylated with a compound of formula (XXXVIII′) containing a leaving group Lg2 such as for example mesylate or bromide. The compound of formula (XLVI) can be transformed into the boronate of formula (XLVII) which can be coupled in a cross-coupling reaction such as a Suzuki reaction with a compound of formula (XLVIII).

The resulting compound of formula (XLV) can be macrocyclized by a nucleophilic substitution reaction. After macrocyclization, the A ring can optionally be substituted resulting in the compound of formula (XLIX). Eventually, final deprotection of the compound of formula (XLIX) gives a particular case of the compound of formula (I/c) wherein Ra represents hydrogen. Alternatively, an alkylation step can be done before final deprotection of Pg1 resulting in particular case of the compound of formula (I/c) wherein Ra is not a hydrogen.

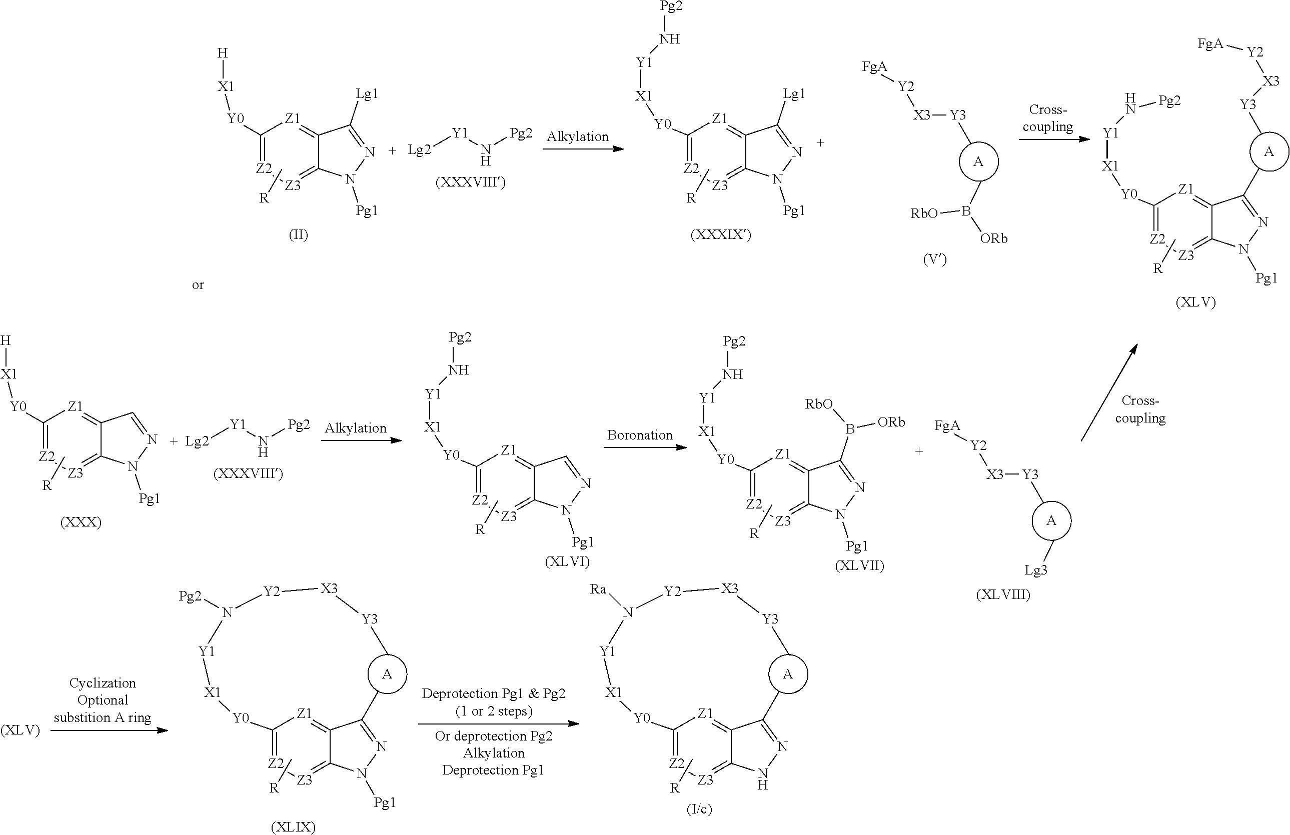

### In the Above Scheme J

Compound of formula (I/c) is a particular case of compound of formula (I) wherein —X2- represents —NRa—.

The alkylation between a compound of formula (II) or a compound of formula (XXX) with a compound of formula (XXXVIII′) can be accomplished in a solvent such as DMF or DMA and a base such as cesium carbonate at RT or at an elevated temperature such as 50° C. or 85° C.

The boronation of the compound of formula (XLVI) can be achieved using bis(pinacolato)diboron, 4,4′-di-tert-butyl-2,2′-bipyridine and 1,5-cyclooctadiene)(methoxy) iridium(I) dimer in a solvent such as dry MTBE at an elevated temperature such as 90° C.

Organometallic cross coupling such as Suzuki coupling of the compound of formula (XXXIX′) or of formula (XLVII) with a compound of formula (V′) or a compound of formula (XLVIII), respectively, can be done using palladium catalysts such as for example tetrakis(triphenylphosphine)palladium(0) combined or not with 2-dicyclohexylphosphino-2′,4′,6′-triisopropylbiphenyl (Xphos) in the presence of potassium phosphate tribasic or sodium carbonate in a solvent mixture such as for example dioxane/water or DME/water at an elevated temperature such as for example 110° C. or at 100° C. under microwave conditions.

The cyclisation of the compound of formula (XLV) can be performed by a substitution reaction using a base such as sodium hydride in a solvent such as DMF at for example RT or using cesium carbonate in a solvent such as DMA at an elevated temperature such as 90° C.

After cyclisation, either or not after substitution of the A ring, the protecting group Pg1 of the fused pyrazolo structure NH and the protecting group Pg2 in the compound of formula (XLIX) can be removed in one step under acidic conditions such as 4N HCl in dioxane or TFA in DCM at room temperature or PTSA in a solvent mixture such as methanol/water at an elevated temperature such as 65° C. to yield the final compound of formula (I/c).

Alternatively, either or not after substitution of the A ring, the protecting group Pg2 in the compound of formula (XLIX) can be removed using thiophenol and a base such as cesium carbonate in a solvent such as DMF at a temperature ranging from 0° C. to RT. The Pg1 of the fused pyrazolo structure NH in the compound of formula (XLIX) can be removed under acidic conditions such as 4N HCl in dioxane or TFA in DCM at room temperature or PTSA in a solvent mixture such as methanol/water at an elevated temperature such as 65° C. to yield the compound of formula (I/c).

Alternatively, either or not after substitution of the A ring, the protecting group Pg2 in the compound of formula (XLIX) can be removed using thiophenol and a base such as cesium carbonate in a solvent such as DMF at a temperature ranging from 0° C. to RT. An alkylation step can be done using standard conditions. The Pg1 of the fused pyrazolo structure NH can be removed under acidic conditions such as 4N HCl in dioxane or TFA in DCM at room temperature or PTSA in a solvent mixture such as methanol/water at an elevated temperature such as 65° C. to yield the compound of formula (I/c).

### Scheme K

The compounds of formula (I/b) and (I/b′) as described in Scheme I, can also be prepared as shown in general Scheme K below wherein the structure of formula (XVII) is alkylated with a compound of formula (L) containing a leaving group Lg2 such as for example mesylate or bromide. After alkylation, the compound of formula (LI) is coupled in a cross-coupling reaction such as a Suzuki reaction with a compound of formula (XII) to yield a compound of formula (LIII) which can be deprotected to give (LIV) that can be alkylated to form a compound of formula (LV). Deprotection of the compound of formula (LV) results in a compound of formula (LVI). The compound of formula (LVI) can macrocyclized by an amidation reaction. After macrocyclization, the A ring of compound (VII″) can optionally be substituted and/or the amide can be reduced to the amine and the amine can be alkylated. Final deprotection of the compound of formula (VII″) gives the compound of formula (I/b).

Alternatively, compound of formula (VII″) can be optionally substituted on the A ring, and/or optionally alkylated to give after Pg1 deprotection compound (I/b′). Compound (I/b′) can be further reduced to give compound (I/b).

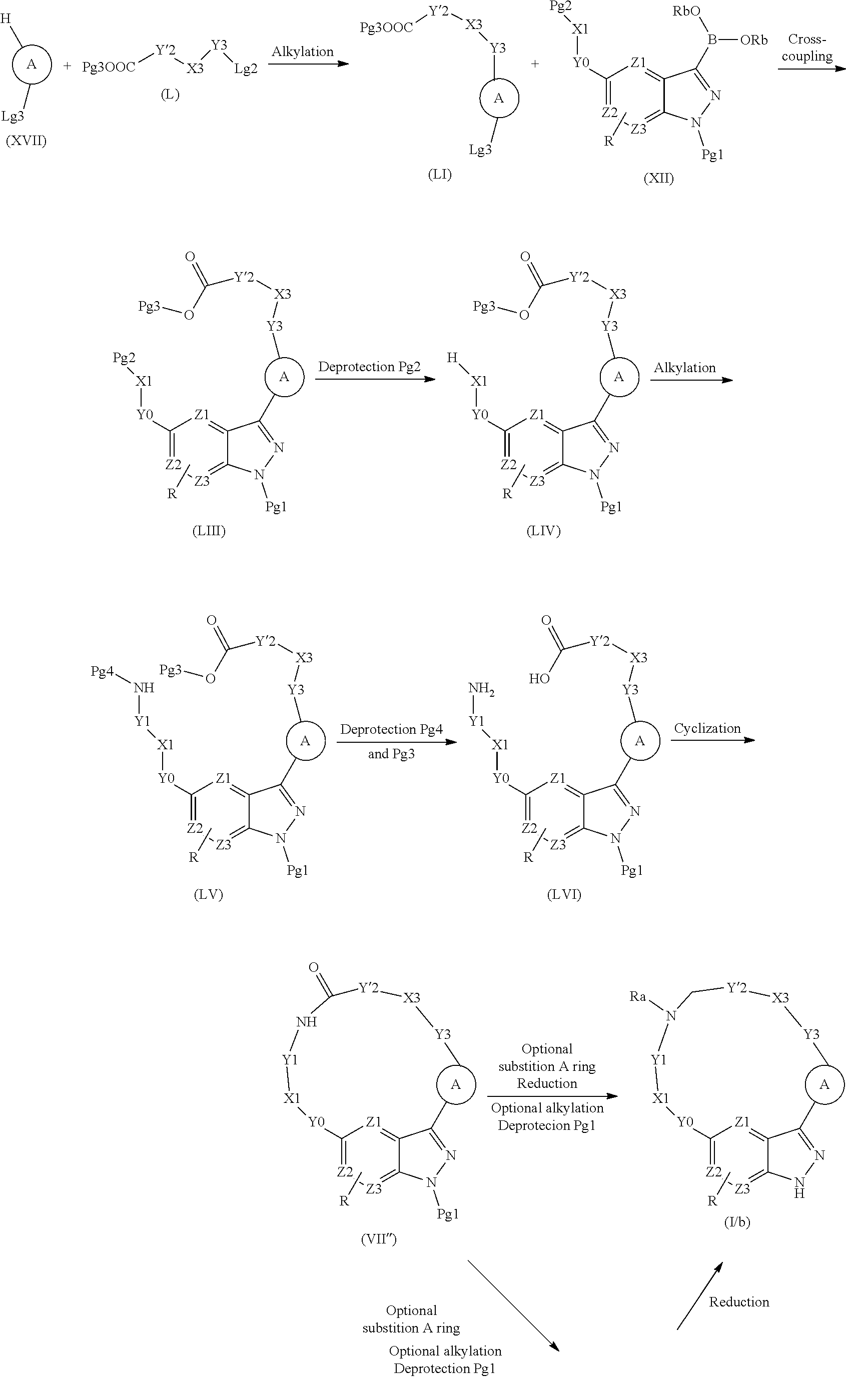

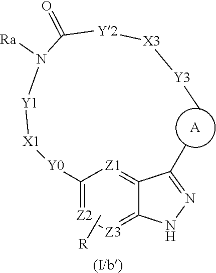

### In the Above Scheme K

The alkylation between a compound of formula (XVII) with a compound of formula (L) can be accomplished in a solvent such as DMF or DMA and a base such as potassium carbonate at temperature such as −10° C. to RT.

Organometallic cross coupling such as Suzuki coupling of the compound of formula (LI) with a compound of formula (XII) or can be done using palladium catalysts such as for example tetrakis(triphenylphosphine)palladium(O) combined or not with 2-dicyclohexylphosphino-2′,4′,6′-triisopropylbiphenyl (Xphos) in the presence of potassium phosphate tribasic or sodium carbonate in a solvent mixture such as for example dioxane/water or DME/water at an elevated temperature such as for example 100° C. or at 100° C. under microwave conditions.

The deprotection of the compound of formula (LIII) can be done using TBAF in a solvent such as THF at a temperature such as RT.

The alkylation of a compound of formula (LIV) can be accomplished using for example a triflate in a solvent such as DMF, DMA or ACN and a base such as cesium carbonate at RT or at an elevated temperature such as 60° C., 90° C. or 100° C.

Deprotection of the compound of formula (LV) can be achieved using hydrogen gas, Pd/C or Pd(OH)₂ in a solvent such as THF at a temperature such as 50° C.

The cyclisation of the compound of formula (LVI) can be performed by an amidation reaction using N-(dimethylamino)-1H-1,2,3-triazolo-4,5-bpyridin-1-ylmethylene-N-methylmethan-aminium hexafluorophosphate N-oxide (HATU) or (benzotriazol-1-yloxy)tripyrrolidino phosphonium hexafluorophosphate (PyBOP) or N,N,N′,N-Tetramethyl-O-(1H-benzotriazol-1-yl)uronium hexafluorophosphate (HBTU), a base such as N,N-diisopropylethylamine (DIPEA) or trimethylamine in a solvent such as DMF or DMA at for example RT.

Reduction of the amide, either or not after substitution of the A ring, can be achieved using borane dimethyl sulfide complex 2N solution in THF at a temperature such as RT or can be done using lithium aluminium hydride (1.0 M solution in THF) and trimethylsilyl chloride in a solvent such as THF at a temperature such as 0° C. and/or RT. An optional alkylation using standard conditions can be performed followed by deprotection of the Pg1 under acidic conditions such as 4N HCl in dioxane or TFA in DCM at room temperature or PTSA in a solvent mixture such as methanol/water at an elevated temperature such as 65° C. to yield the compound of formula (I/b). Alternatively, compound (I/b) can be prepared after reduction of compound (I/b′) under standard conditions. With (I/b′) being synthesized from compound (VII″) after optional A ring substitution and/or an optional alkylation under standard conditions followed by Pg1 deprotection.

### EXAMPLES

IUPAC names of compounds of the invention were generated using the following software:
Product version: MarvinSketch 19.18.0
Build Date: 2019 Jul. 30
Internal Build id: 19.18.0-10265
Operating System: amd64 Windows 10.10.0
Character encoding: windows-1252
Java: Jeroen Frijters Java 1.8.0
Memory: 55.8M total, 10.0M free
Environment: Application
NET Version: v2.0.50727
IKVM Version: 8.10.1.11
JChem NET API Assembly Version: 19.18.0.10265
JChem NET API File Version: 19.18.0.10265
Marvin .NET Version: 19.18.0.339
Process type: x64
http://www.chemaxon.com

In case of a discrepancy between the drawn chemical structures and the corresponding chemical names, the drawn chemical structures will be considered as true structures.

To prepare the compounds described in the examples, the following experimental protocols were followed unless otherwise indicated.

Unless otherwise stated, reaction mixtures were stirred magnetically at room temperature. When organic solutions were “dried”, they were generally dried over a drying agent such as sodium sulfate or magnesium sulfate. When mixtures, solutions and extracts were “concentrated”, they were typically concentrated on a rotary evaporator under reduced pressure.

All intermediates and final exemplified compounds were analyzed by high-performance liquid chromatography (HPLC) following one of the described methods below.

### LCMS Method A

Analyses were carried out on a Thermo Scientific Accucore C18 (50 mm long×2.1 mm I.D., 2.6 μm) at 35° C., with a flow rate of 1.50 mL/min. A gradient elution was performed from 95% (Water+0.1% Formic acid)/5% Acetonitrile to 5% (Water+0.1% Formic acid)/95% Acetonitrile in 1.30 minutes; the resulting composition was held for 0.5 min; then the final mobile phase composition; from 5% (Water+0.1% Formic acid)/95% Acetonitrile to 90% (Water+0.1% Formic acid)/10% Acetonitrile in 0.10 minutes. The injection volume was 1 μL. MS acquisition range and UV detector were set to 100-1000 m/z and 190-400 nm respectively.

### LCMS Method B

Analyses were carried out on a Phenomenex Kinetex 00B-4475-AN C18 column (50 mm long×2.1 mm I.D.; 1.7 μm particles) at 60° C., with a flow rate of 1.5 mL/min. A gradient elution was performed from 90% (Water+0.1% Formic acid)/10% Acetonitrile to 10% (Water+0.1% Formic acid)/90% Acetonitrile in 1.50 minutes; the resulting composition was held for 0.40 min; then the final mobile phase composition; from 10% (Water+0.1% Formic acid)/90% Acetonitrile to 90% (Water+0.1% Formic acid)/10% Acetonitrile in 0.10 minutes. The injection volume was 2 μL with Agilent autosampler injector or 5 μL with Gerstel MPS injector. MS acquisition range and DAD detector were set to 100-800 m/z and 190-400 nm respectively.

LCMS method C

Analyses were carried out on an YMC pack ODS-AQ C18 column (50 mm long×4.6 mm ID.; 3 μm particle size) at 35° C., with a flow rate of 2.6 mL/min. A gradient elution was performed from 95% (Water+0.1% Formic acid)/5% Acetonitrile to 5% (Water+0.1% Formic acid)/95% Acetonitrile in 4.8 min; the resulting composition was held for 1.0 min; from 5% (Water+0.1% formic acid)/95% Acetonitrile to 95% (Water+0.1% formic acid)/5% Acetonitrile in 0.2 min. The standard injection volume was 2 μL. Acquisition ranges were set to 190-400 nm for the UV-PDA detector and 100-1400 m/z for the TOF-LCMS detector. Total run time: 6.2 minutes.

### LCMS Method D

Analyses were carried out on a Phenomenex Kinetex C18 column (50 mm long×2.1 mm ID.; 2.6 μm particle size) at 35° C., with a flow rate of 0.7 mL/min. A gradient elution was performed from 95% (Water+50 mM Ammonium Acetate)/5% Acetonitrile to 5% (Water+50 mM Ammonium Acetate)/95% Acetonitrile in 4.8 min; the resulting composition was held for 1.0 min; from 5% (Water+50 mM Ammonium Acetate)/95% Acetonitrile to 95% (Water+50 mM Ammonium Acetate)/5% Acetonitrile in 0.2 min. The standard injection volume was 2 μL. Acquisition ranges were set to 190-400 nm for the UV-PDA detector and 100-1400 m/z for the MS detector. Total run time: 6.2 minutes.

LCMS method E

Analyses were carried out on an YMC pack ODS-AQ C18 column (50 mm long×4.6 mm ID.; 3 μm particle size) at 35° C., with a flow rate of 2.6 mL/min. A gradient elution was performed from 95% (Water+0.1% Formic acid)/5% Acetonitrile to 5% (Water+0.1% Formic acid)/95% Acetonitrile in 4.8 min; the resulting composition was held for 1.0 min; from 5% (Water+0.1% formic acid)/95% Acetonitrile to 95% (Water+0.1% formic acid)/5% Acetonitrile in 0.2 min.

The standard injection volume was 2 μL. Acquisition ranges were set to 190-400 nm for the UV-PDA detector and 100-1400 m/z for the MS detector.

### LCMS Method F (Current 20V)

Analytical HPLC was conducted on a X-Select CSH C18 XP column (2.5 μm 30×4.6 mm id) eluting with 0.1% formic acid in water (solvent A) and 0.1% formic acid in acetonitrile (solvent B), using the following elution gradient 0-3 minutes: 5% to 100% B, 3-4 minutes 100% B, at a flow rate of 1.8 mL/minute at 40° C. The mass spectra (MS) were recorded on a Waters ZQ mass spectrometer (scan 200-900 uma) using electrospray positive ionisation [ES+ to give [M+H]⁺ molecular ions] or electrospray negative ionisation [ES− to give [M−H]⁻ molecular ions] modes with a 20 V cone voltage.

### LCMS Method G (Basic Current 20V)

Analytical HPLC was conducted on a X-Select CSH C18 XP column (2.5 μm 30×4.6 mm id) eluting with (NH₄)₂CO₃ aq. 2 g/L in water (solvent A) and acetonitrile (solvent B), using the following elution gradient 0-3 minutes: 5% to 100% B, 3-4 minutes 100% B, at a flow rate of 1.8 mL/minute at 40° C. The mass spectra (MS) were recorded on a Waters ZQ mass spectrometer (scan 200-900 uma) using electrospray positive ionisation [ES+ to give [M+H]⁺ molecular ions] or electrospray negative ionisation [ES− to give [M−H]⁻ molecular ions] modes with a 20 V cone voltage.

### LCMS Method H (Polar 20V Current)

Analytical HPLC was conducted on a X-Select CSH C18 XP column (2.5 μm 30×4.6 mm id) eluting with 0.1% formic acid in water (solvent A) and 0.1% formic acid in acetonitrile (solvent B), using the following elution gradient 0-4 minutes: 0% to 50% B at a flow rate of 1.8 mL/minute at 40° C. The mass spectra (MS) were recorded on a Waters ZQ mass spectrometer (scan 200-900 uma) using electrospray positive ionisation [ES+ to give [M+H]⁺ molecular ions] or electrospray negative ionisation [ES− to give [M−H]⁻ molecular ions] modes with a 20 V cone voltage.

### LCMS Method I (Apolar 20V Current)

Analytical HPLC was conducted on a X-Select CSH C18 XP column (2.5 μm 30×4.6 mm id) eluting with 0.1% formic acid in water (solvent A) and 0.1% formic acid in acetonitrile (solvent B), using the following elution gradient 0-4 minutes: 40% to 100% B, 4-5 min: 100% B at a flow rate of 1.8 mL/minute at 40° C. The mass spectra (MS) were recorded on a Waters ZQ mass spectrometer (scan 200-900 uma) using electrospray positive ionisation [ES+ to give [M+H]⁺ molecular ions] or electrospray negative ionisation [ES− to give [M−H]⁻ molecular ions] modes with a 20 V cone voltage.

LCMS Method J (Current 20V 7 min)

Analytical HPLC was conducted on a X-Select CSH C18 XP column (2.5 μm 30×4.6 mm id) eluting with 0.1% formic acid in water (solvent A) and 0.1% formic acid in acetonitrile (solvent B), using the following elution gradient 0-6 minutes: 5% to 100% B, 6-7 minutes 100% B, at a flow rate of 1.8 ml/minute at 40° C. The mass spectra (MS) were recorded on a Waters ZQ mass spectrometer (scan 200-900uma) using electrospray positive ionisation [ES+ to give MH⁺ molecular ions] or electrospray negative ionisation [ES− to give (M−H)− molecular ions] modes with a 20V cone voltage.

### LCMS Method K (Current 40V)

Analytical HPLC was conducted on a X-Select CSH C18 XP column (2.5 μm 30×4.6 mm id) eluting with 0.1% formic acid in water (solvent A) and 0.1% formic acid in acetonitrile (solvent B), using the following elution gradient 0-3 minutes: 5% to 100% B, 3-4 minutes 100% B, at a flow rate of 1.8 ml/minute at 40° C. The mass spectra (MS) were recorded on a Waters ZQ mass spectrometer (scan 200-900uma) using electrospray positive ionisation [ES+ to give MH⁺ molecular ions] or electrospray negative ionisation [ES− to give (M−H)− molecular ions] modes with a 40V cone voltage.

### LCMS Method L (Current 5V)

Analytical HPLC was conducted on a X-Select CSH C18 XP column (2.5 μm 30×4.6 mm id) eluting with 0.1% formic acid in water (solvent A) and 0.1% formic acid in acetonitrile (solvent B), using the following elution gradient 0-3 minutes: 5% to 100% B, 3-4 minutes 100% B, at a flow rate of 1.8 ml/minute at 40° C. The mass spectra (MS) were recorded on a Waters ZQ mass spectrometer (scan 200-900uma) using electrospray positive ionisation [ES+ to give MH⁺ molecular ions] or electrospray negative ionisation [ES− to give (M−H)− molecular ions] modes with a 40V cone voltage.

LCMS Method M (Apolar 20V Current 7 min)

Analytical HPLC was conducted on a X-Select CSH C18 XP column (2.5 μm 30×4.6 mm id) eluting with 0.1% formic acid in water (solvent A) and 0.1% formic acid in acetonitrile (solvent B), using the following elution gradient 0-6 minutes: 40% to 100% B, 6-7 min: 100% B at a flow rate of 1.8 ml/minute at 40° C. The mass spectra (MS) were recorded on a Waters ZQ mass spectrometer (scan 200-900uma) using electrospray positive ionisation [ES+ to give MH⁺ molecular ions] or electrospray negative ionisation [ES− to give (M−H)− molecular ions] modes with a 20V cone voltage.

LCMS Method N (Basic Current 20V 7 min)

Analytical HPLC was conducted on a X-Select CSH C18 XP column (2.5 μm 30×4.6 mm id) eluting with (NH₄)₂CO₃ aq. 2 g/L in water (solvent A) and acetonitrile (solvent B), using the following elution gradient 0-6 minutes: 5% to 100% B, 6-7 minutes 100% B, at a flow rate of 1.8 mL/minute at 40° C. The mass spectra (MS) were recorded on a Waters ZQ mass spectrometer (scan 200-900 uma) using electrospray positive ionisation [ES+ to give [M+H]⁺ molecular ions] or electrospray negative ionisation [ES− to give [M−H]⁻ molecular ions] modes with a 20 V cone voltage.

### LCMS Method O

All analyses were performed using an Agilent 6120 LC/MSD quadrupole coupled to an Agilent 1200 series liquid chromatography (LC) system consisting of a quaternary pump with degasser, autosampler, thermostated column compartment and diode array detector. The mass spectrometer (MS) was operated with an atmospheric pressure electro-spray ionization (API-ES) source in positive ion mode. The capillary voltage was set to 3000 V, the fragmentor voltage to 70 V and the quadrupole temperature was maintained at 100° C. The drying gas flow and temperature values were 12.0 L/min and 350° C. respectively. Nitrogen was used as the nebulizer gas, at a pressure of 35 psig. Data acquisition was performed with Agilent Chemstation software

Analyses were carried out on a Phenomenex Gemini C18 column (100 mm long×4.6 mm I.D.; 5 μm particles) at 25° C., with a flow rate of 3.014 mL/min. A gradient elution was performed from 95% of a NH4OAc aqueous solution (65 mM+Acetonitrile 9:1)/5% (Acetonitrile/Methanol 1:1) to 45% of a NH4OAc aqueous solution (65 mM+Acetonitrile 10%)/55% (Acetonitrile/Methanol 1:1) in 4.56 min to 0% of a NH4OAc aqueous solution (65 mM+Acetonitrile 10%)/100% (Acetonitrile/Methanol 1:1) in 0.6 min.; the resulting composition was held for 1.14 min; from 100% (Acetonitrile/Methanol 1:1) to 95% NH4OAc aqueous solution (65 mM+Acetonitrile 9:1)/5% (Acetonitrile/Methanol 1:1) in 0.3 min. The standard injection volume was 5 μL. Acquisition ranges were set to 200-400 nm for the UV-PDA detector and 100-1000 m/z for the MS detector.

GCMS Method a (20 minMB)

All analyses were performed using an Agilent 6850 Gas Chromatography (GC) system, consisting of autosampler, oven and thermostated column, coupled to an Agilent 5975C quadrupole mass spectrometer (MS) with a diffusion pump. The MS was operated with an electronic impact (EI) ionization source in positive ion mode. The quadrupole and the ionization source temperatures were set at 150 and 230° C., respectively. Helium was used as carrier gas. Data acquisition was performed with Agilent Chemstation software.

Analyses were carried out on a HP-5MS 5% Phenyl Methyl Siloxane column, (30.0 μm length×250 μm diameter×0.25 μm film thickness), with a flow rate of 1.0 mL/min, an initial pressure of 0.6 bar, and an average velocity of 37 cm/s. An oven ramp was performed as follows: The temperature was held at 70° C. for 2.0 min; from 70° C. to 270° C. in 10.0 min; the temperature was held at 270° C. for 8.0 min. The injection volume was 1 μL. MS acquisition range was set to 45-500 m/z.

Chiral analytical SFC was conducted on a Whelk O1 (R,R) column (1.8 μm 100×4.6 mmid) eluting with CO₂/methanol (70/30) at a flow rate of 2.5 mL/minute at 35° C.

All final exemplified compounds were analysed by proton NMR.

¹H NMR spectra were recorded in either CDCl₃, d6-DMSO or CD₃OD on a Bruker Avance 400 MHz or were recorded on a Bruker Ultrashield AV300 MHz spectrometer, with a Bruker 5 mm BBI 1H/D-BB Z-GRD probe, using a BACS-60 sample changer, and registered with Bruker Topspin 2.1 software. Chemical shifts are reported in parts per million (ppm) relative to the residual protiated solvent (7.26 ppm for CDCl₃, 2.50 ppm for d6-DMSO and 3.31 ppm for CD₃OD). For ¹H NMR spectra, multiplicities, coupling constants in hertz and numbers of protons are indicated parenthetically. Abbreviations for NMR data are as follows: s=singlet, d=doublet, t=triplet, q=quadruplet, m=multiplet, br s=broad singlet.

### Abbreviations

The following abbreviations are employed herein:
Ph=phenyl
Ac=acetate
Bn=benzyl
t-Bu=tert-butyl
n-Bu=linear butyl
Me=methyl
Et=ethyl
Pr=propyl
iPr=isopropyl
Bu=butyl
TMS=trimethylsilyl
TBS=tert-butyldimethylsilyl
TFA=trifluoroacetic acid
i-Pr₂NEt or DIPEA=N,N-diisopropylethylamine
TEA=triethylamine
DMAP=4-dimethylaminopyridine
Pd/C=palladium on carbon
KOH=potassium hydroxide
NaOH=sodium hydroxide
LiOH=lithium hydroxide
Ar=argon
N₂=nitrogen
H₂=hydrogen
LAH=lithium Aluminium Hydride
Boc=tert-butoxycarbonyl
Cbz=carboxybenzyl
LDA=lithium diisopropylamide
NBS=N-bromosuccinimide
NIS=N-iodosuccinimide
ACN=acetonitrile
PTSA=p-toluenesulfonic acid
THF=tetrahydrofuran
DCM=dichloromethane
DMF=N,N-dimethylformamide
AA=acetic acid
TBME=methyl tert-butyl ether
Hept=heptane
EtOAc=ethyl acetate
DHP=3,4-Dihydro-2H-pyran
THP=Tetrahydropyran
TBAF=tetrabutylammonium fluoride
cataCXium=di(1-adamantyl)-n-butylphosphine
XPhos=2-Dicyclohexylphosphino-2′,4′,6′-triisopropylbiphenyl
dppf=1,1′-Bis(diphenylphosphino)ferrocene
wt %=weight %
e.e.=enantiomeric excess
min=minute(s)
h or hr=hour(s)
L=liter(s)
mL=milliliter(s)
μL=microliter(s)
g=gram(s)
mg=milligram(s)
mol=moles
mmol=millimole(s)
RT=room temperature
tR=retention time
sat=saturated
aq.=aqueous
TLC=thin layer chromatography
HPLC=high performance liquid chromatography
LC/MS=high performance liquid chromatography/mass spectrometry
MS or Mass Spec=mass spectrometry
NMR=nuclear magnetic resonance
ppm=parts per million
Pd₂(dba)₃=tris(dibenzylideneacetone)dipalladium(0)

### Example 1: 4-(morpholin-4-yl)-7,10-dioxa-13,18,19-triazatetracyclo[12.5.2.1^{2,6}.0^{17,20}] docosa-1(19),2(22),3,5,14(21),15,17(20)-heptaene

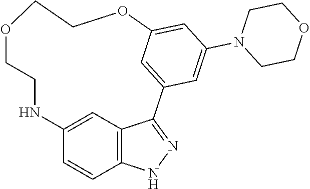 [SMILES: c1cc2[nH]nc3c2cc1NCCOCCOc1cc-3cc(N2CCOCC2)c1]

Example 1 is prepared according to the synthesis route described in general Scheme A.

### Preparation of Intermediate 1: 3-iodo-5-nitro-1H-indazole

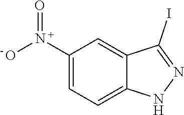

To a solution of 5-nitro-1H-indazole (5 g, 30.65 mmol) in DMF (92 mL) was added N-Iodosuccinimide (7.24 g, 32.18 mmol). The reaction mixture was stirred at RT for 4 days. The reaction mixture was diluted with a saturated aqueous NaHCO₃ solution and extracted with DCM. The organic layer was dried over anhydrous magnesium sulfate, filtered and the solvent was removed under reduced pressure. The solid was washed with water and filtered yielding 3-iodo-5-nitro-1H-indazole as a salmon solid which was used in the next step without further purification.

LCMS method B: [M+H]⁺=290.0, t_{R}=0.650 min.

### Preparation of Intermediate 2: 3-iodo-5-nitro-1-tetrahydropyran-2-yl-indazole

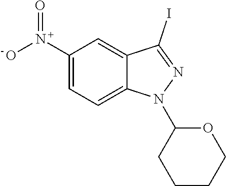

DHP (9.49 mL, 103.8 mmol) and p-toluenesulfonic acid monohydrate (0.98 g, 5.19 mmol) were added to a solution of 3-iodo-5-nitro-1H-indazole (7.50 g, 25.95 mmol) in DCM (78 mL) at RT. The reaction mixture was stirred at RT for 16 h. Additional DHP (2.37 mL, 25.95 mmol) and p-toluenesulfonic acid monohydrate (0.49 g, 2.595 mmol) were added and mixture was stirred at RT for 5 h. The reaction mixture was diluted with a saturated aqeuous NaHCO₃ solution and extracted with DCM. The combined organic layers were dried over anhydrous magnesium sulfate, filtered and the solvent was removed under reduced pressure. The crude was triturated with heptane and filtered off, yielding 3-iodo-5-nitro-1-tetrahydropyran-2-yl-indazole as a pale yellow solid which was used in the next step without further purification.

LCMS method B: [M+H]⁺=373.9, t_{R}=1.088 min.

### Preparation of Intermediate 3: 3-iodo-1-tetrahydropyran-2-yl-indazol-5-amine

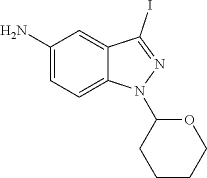 [SMILES: Nc1ccc2c(c1)c(I)nn2C1CCCCO1]

3-iodo-5-nitro-1-tetrahydropyran-2-yl-indazole (7.079 g, 18.97 mmol) and ammonium chloride (1.015 g, 18.97 mmol) were dissolved in a mixture of ethanol (45.6 mL) and water (11.4 mL). The reaction mixture was stirred at 35° C. for 15 min. Iron (5.297 g, 94.85 mmol) was added and the mixture was stirred at 50° C. for 1 h and at RT for 16 h. The mixture was filtered over a path of celite and was extracted with ethyl acetate. The organic layer was separated, dried over anhydrous magnesium sulfate, filtered and the solvent was removed under reduced pressure. The crude was purified by silica gel column chromatography using DCM/EtOAc 100/0 to 80/20 as eluent. The desired fractions were combined and the solvent was removed under reduced pressure, yielding 3-iodo-1-tetrahydropyran-2-yl-indazol-5-amine as a salmon oil, that solidified upon standing.

LCMS method B: [M+H]⁺=344.0, t_{R}=0.496 min.

### Preparation of Intermediate 4: N-(3-iodo-1-tetrahydropyran-2-yl-indazol-5-yl)-2-nitro-benzenesulfonamide

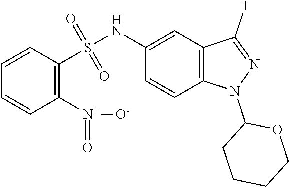 [SMILES: O=[N+]([O-])c1ccccc1S(=O)(=O)Nc1ccc2c(c1)c(I)nn2C1CCCCO1]

3-iodo-1-tetrahydropyran-2-yl-indazol-5-amine (5.75 g, 16.76 mmol), pyridine (2.706 mL, 33.52 mmol) and DMAP (205 mg, 1.676 mmol) were dissolved in dioxane (25 mL). 2-nitrobenzenesulfonyl chloride (5.572 g, 25.14 mmol) in dioxane (25 mL) was added slowly at 0° C. The reaction mixture was stirred for 16 h. The suspension was diluted with DCM and extracted with a saturated aqueous NaHCO₃ solution. The organic layer was separated, dried over anhydrous magnesium sulfate, filtered and the solvent was removed under reduced pressure. The crude was purified by silica gel column chromatography using heptane/ethyl acetate 100/0 to 60/40 as eluent. The solid was triturated with heptane and filtered to afford N-(3-iodo-1-tetrahydropyran-2-yl-indazol-5-yl)-2-nitro-benzenesulfonamide as a pink/orange solid.

LCMS method A: [M+H]⁺=528.8, t_{R}=0.945 min.

### Preparation of Intermediate 5: tert-butyl-[2-(2-chloroethoxy)ethoxy]-dimethyl-silane

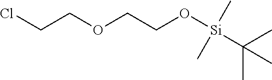

To a solution of 2-(2-chloroethoxy)ethanol (5 g, 40.14 mmol) and triethylamine (5.6 mL, 41.14 mmol) in THF (120 mL) was added tert-butyldimethylsilyl chloride (6.05 g, 40.14 mmol). The reaction mixture was stirred at RT for 16 h. Additional tert-butyldimethylsilyl chloride (1.21 g, 8.02 mmol) and triethylamine (1.1 mL, 8.02 mmol) were added and the mixture was stirred at RT for 23 h. Ethyl acetate was added and the mixture was washed with water then brine. The organic layer was dried over anhydrous magnesium sulfate, filtered and the solvent was removed under reduced pressure to afford tert-butyl-[2-(2-chloroethoxy)ethoxy]-dimethyl-silane as a pale yellow oil which was used in the next step without further purification.

LCMS method B: [M+H]⁺=239.1, t_{R}=1.144 min.

### Preparation of Intermediate 6: N-[2-[2-[tert-butyl(dimethyl)silyl]oxyethoxy]ethyl]-N-(3-iodo-1-tetrahydropyran-2-yl-indazol-5-yl)-2-nitro-benzenesulfonamide

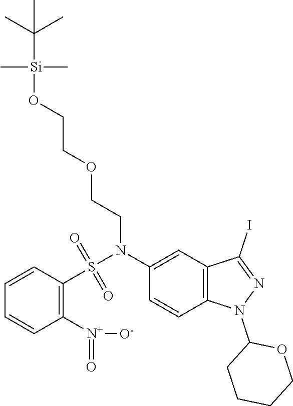 [SMILES: CC(C)(C)[Si](C)(C)OCCOCCN(c1ccc2c(c1)c(I)nn2C1CCCCO1)S(=O)(=O)c1ccccc1[N+](=O)[O-]]

A mixture of N-[3-iodo-1-(oxan-2-yl)-1H-indazol-5-yl]-2-nitrobenzene-1-sulfonamide (700 mg, 1.32 mmol), tert-butyl-[2-(2-chloroethoxy)ethoxy]-dimethyl-silane (473 mg, 1.98 mmol), cesium carbonate (1.29 g, 3.96 mmol) and potassium iodide (219 mg, 1.32 mmol) in DMA (8 mL) was stirred at 100° C. for 16 h. The mixture was diluted with water and extracted with ethyl acetate. The organic layer was separated, dried over anhydrous magnesium sulfate, filtered and the solvent was removed under reduced pressure. The crude was purified by silica gel column chromatography using heptane/ethyl acetate 100/0 to 70/30 as eluent to afford N-[2-[2-[tert-butyl(dimethyl)silyl]oxyethoxy]ethyl]-N-(3-iodo-1-tetrahydropyran-2-yl-indazol-5-yl)-2-nitro-benzene sulfonamide.

LCMS method B: [M+H]⁺=731.2, t_{R}=1.387 min.

### Preparation of Intermediate 8: N-[2-[2-[tert-butyl(dimethyl)silyl]oxyethoxy]ethyl]-N-[3-(3-chloro-5-hydroxy-phenyl)-1-tetrahydropyran-2-yl-indazol-5-yl]-2-nitro-benzenesulfonamide

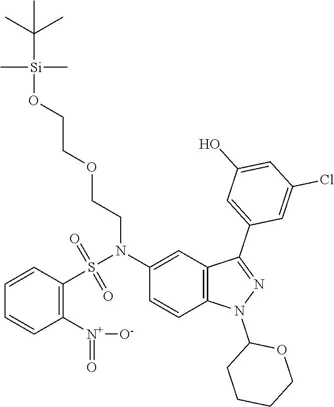 [SMILES: CC(C)(C)[Si](C)(C)OCCOCCN(c1ccc2c(c1)c(-c1cc(O)cc(Cl)c1)nn2C1CCCCO1)S(=O)(=O)c1ccccc1[N+](=O)[O-]]

The reaction was performed in two batches.

### Batch 1

To a degassed solution of N-[2-[2-[tert-butyl(dimethyl)silyl]oxyethoxy]ethyl]-N-(3-iodo-1-tetrahydropyran-2-yl-indazol-5-yl)-2-nitro-benzenesulfonamide (1.10 g, 1.51 mmol), 3-chloro-5-(4,4,5,5-tetramethyl-1,3,2-dioxaborolan-2-yl)phenol (578 mg, 2.27 mmol) and potassium triphosphate (961 mg, 4.53 mmol) in dioxane (13.5 mL) and water (4.5 mL) were added tetrakis(triphenylphosphine)palladium(0) (87 mg, 0.0755 mmol) and XPhos (72 mg, 0.151 mmol). The reaction mixture was stirred at 90° C. for 16 h. The organic layer was separated, dried over anhydrous magnesium sulfate, filtered and the solvent was removed under reduced pressure. The crude was joined with batch 2 for purification.

### Batch 2

To a degassed solution of N-[2-[2-[tert-butyl(dimethyl)silyl]oxyethoxy]ethyl]-N-(3-iodo-1-tetrahydropyran-2-yl-indazol-5-yl)-2-nitro-benzenesulfonamide (576 mg, 0.79 mmol), 3-chloro-5-(4,4,5,5-tetramethyl-1,3,2-dioxaborolan-2-yl)phenol (300 mg, 1.18 mmol) and potassium triphosphate (503 mg, 2.37 mmol) in dioxane (1.8 mL) and water (0.6 mL) were added tetrakis(triphenylphosphine)palladium(0) (46 mg, 0.0395 mmol) and XPhos (38 mg, 0.079 mmol). The reaction mixture was stirred at 90° C. for 16 h. The organic layer was separated, dried over anhydrous magnesium sulfate, filtered and the solvent was removed under reduced pressure. The combined crude compounds from batch 1 and batch 2 were purified by silica gel column chromatography using heptane/ethyl acetate 100/0 to 0/100 as eluent to afford N-[2-[2-[tert-butyl(dimethyl)silyl]oxyethoxy]ethyl]-N-[3-(3-chloro-5-hydroxy-phenyl)-1-tetrahydropyran-2-yl-indazol-5-yl]-2-nitro-benzenesulfonamide as an orange oil.

LCMS method B: [M+H]⁺=731.3-733.3, t_{R}=1.384 min

### Preparation of Intermediate 9: N-[3-(3-chloro-5-hydroxy-phenyl)-1-tetrahydro pyran-2-yl-indazol-5-yl]-N-[2-(2-hydroxyethoxy)ethyl]-2-nitro-benzenesulfonamide

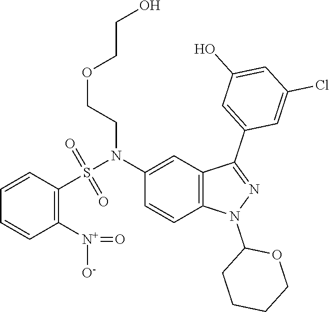 [SMILES: O=[N+]([O-])c1ccccc1S(=O)(=O)N(CCOCCO)c1ccc2c(c1)c(-c1cc(O)cc(Cl)c1)nn2C1CCCCO1]

To a solution of N-[2-[2-[tert-butyl(dimethyl)silyl]oxyethoxy]ethyl]-N-[3-(3-chloro-5-hydroxy-phenyl)-1-tetrahydropyran-2-yl-indazol-5-yl]-2-nitro-benzenesulfonamide (700 mg, 0.96 mmol) in THF (2.88 mL) was added TBAF (1M in THF, 2.88 mL, 2.88 mmol) at 0° C. The reaction mixture was stirred at RT for 21 h. The mixture was washed with a saturated aqueous NaHCO₃ solution and extracted with ethyl acetate. The organic layer was separated, dried over anhydrous magnesium sulfate, filtered and the solvent was removed under reduced pressure. The residue was purified by silica gel column chromatography using heptane/ethyl acetate 100/0 to 20/80 as eluent to afford N-[3-(3-chloro-5-hydroxy-phenyl)-1-tetrahydropyran-2-yl-indazol-5-yl]-N-[2-(2-hydroxyethoxy)ethyl]-2-nitro-benzenesulfonamide as a pale pink foam.

LCMS method B: [M+H]⁺=617.2-619.2, t_{R}=0.989 min.

### Preparation of Intermediate 10: N-[2-(2-chloroethoxy)ethyl]-N-[3-(3-chloro-5-hydroxy-phenyl)-1-tetrahydropyran-2-yl-indazol-5-yl]-2-nitro-benzenesulfonamide

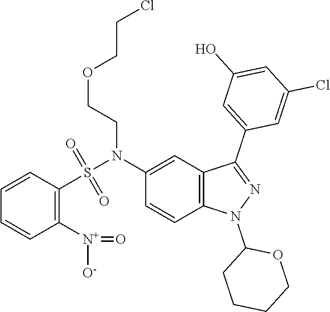 [SMILES: O=[N+]([O-])c1ccccc1S(=O)(=O)N(CCOCCCl)c1ccc2c(c1)c(-c1cc(O)cc(Cl)c1)nn2C1CCCCO1]

To a solution of N-[3-(3-chloro-5-hydroxy-phenyl)-1-tetrahydropyran-2-yl-indazol-5-yl]-N-[2-(2-hydroxyethoxy)ethyl]-2-nitro-benzenesulfonamide (442 mg, 0.72 mmol) and pyridine (465 μL, 5.76 mmol) in the dry DCM (2.16 mL) at 0° C. was added thionyl chloride (315 μl, 4.32 mmol). The reaction mixture was stirred at RT for 20 h. The mixture was washed with a saturated aqueous NaHCO₃ solution and extracted with ethyl acetate. The organic layer was separated, dried over anhydrous magnesium sulfate, filtered and the solvent was removed under reduced pressure. The crude product was purified by silica gel column chromatography using heptane/ethyl acetate 100/0 to 0/100 as eluent to afford N-[2-(2-chloroethoxy)ethyl]-N-[3-(3-chloro-5-hydroxy-phenyl)-1-tetrahydropyran-2-yl-indazol-5-yl]-2-nitro-benzenesulfonamide as a yellow oil.

LCMS method B: [M+H]⁺=635.2-637.2, t_{R}=1.1 min.

### Preparation of Intermediate 11: 4-chloro-13-(2-nitrobenzenesulfonyl)-18-(oxan-2-yl)-7,10-dioxa-13,18,19-triazatetracyclo[12.5.2.1^{2,6}.0^{17,20}]docosa-1(19),2(22),3,5, 14(21),15,17(20)-heptaene

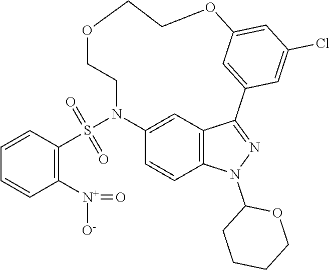 [SMILES: O=[N+]([O-])c1ccccc1S(=O)(=O)N1CCOCCOc2cc(Cl)cc(c2)-c2nn(C3CCCCO3)c3ccc1cc23]

A solution of N-[2-(2-chloroethoxy)ethyl]-N-[3-(3-chloro-5-hydroxy-phenyl)-1-tetrahydro pyran-2-yl-indazol-5-yl]-2-nitro-benzenesulfonamide (283 mg, 0.45 mmol) in dry DMF (70 mL) was added dropwise to a stirred suspension of Cs₂CO₃ (733 mg, 2.25 mmol) in dry DMF (65 mL) at 90° C. The reaction mixture was stirred at 90° C. for 3 h. The solvent was removed under reduced pressure and the residue was diluted with ethyl acetate and washed with a saturated aqueous NaHCO₃ solution. The organic layer was separated, dried over anhydrous magnesium sulfate, filtered and the solvent was removed under reduced pressure. The residue was purified by silica gel column chromatography using heptane/ethyl acetate 100/0 to 70/30/as eluent to afford 4-chloro-13-(2-nitrobenzenesulfonyl)-18-(oxan-2-yl)-7,10-dioxa-13,18,19-triazatetracyclo[12.5.2.1^{2,6}.0^{17,20}]docosa-1(19),2(22),3,5,14(21),15,17(20)-heptaene as a pale yellow solid.

LCMS method B: [M+H]⁺=599.2-601.2, t_{R}=1.243 min.

### Preparation of Intermediate 12: 4-(morpholin-4-yl)-18-(oxan-2-yl)-7,10-dioxa-13,18,19-triazatetracyclo[12.5.2.1^{2,6}.0^{17,20}]docosa-1(19),2(22),3,5,14(21),15,17(20)-heptaene

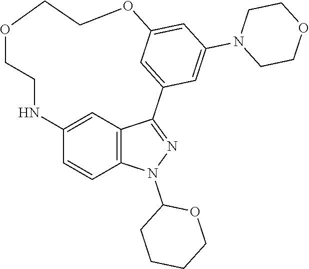 [SMILES: c1cc2c3cc1NCCOCCOc1cc(cc(N4CCOCC4)c1)-c3nn2C1CCCCO1]

A suspension, of 4-chloro-13-(2-nitrobenzenesulfonyl)-18-(oxan-2-yl)-7,10-dioxa-13,18,19-triazatetracyclo[12.5.2.1^{2,6}.0^{17,20}]docosa-1(19),2(22),3,5,14(21),15,17(20)-heptaene (146 mg, 0.24 mmol), morpholine (62 μL, 0.72 mmol) and Cs₂CO₃ (469 mg, 1.44 mmol) DMA (1.44 mL) was degassed with N₂. Palladium(II) acetate (22 mg, 0.024 mmol) and Pd(dppf)Cl₂ (23 mg, 0.048 mmol) were added and the reaction mixture was stirred at 150° C. for 16 h. The reaction mixture was filtered over a pad of celite and washed with ethyl acetate. The solvent was removed under reduced pressure. The crude product was purified by silica gel column chromatography using dichloromethane/ethyl acetate 100/0 to 90/10 as eluent to afford 4-(morpholin-4-yl)-18-(oxan-2-yl)-7,10-dioxa-13,18,19-triazatetracyclo[12.5.2.1^{2,6}.0^{17,20}] docosa-1(19),2(22),3,5,14(21),15,17(20)-heptaene as an orange oil.

LCMS method B: [M+H]⁺=465.2, t_{R}=0.895 min.

### Preparation of Example 1: 4-(morpholin-4-yl)-7,10-dioxa-13,18,19-triazatetracyclo [12.5.2.1^{2,6}.0^{17,20}]docosa-1(19),2(22),3,5,14(21),15,17(20)-heptaene

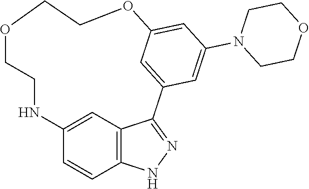 [SMILES: c1cc2[nH]nc3c2cc1NCCOCCOc1cc-3cc(N2CCOCC2)c1]

A mixture of 4-(morpholin-4-yl)-18-(oxan-2-yl)-7,10-dioxa-13,18,19-triazatetracyclo [12.5.2.1^{2,6}.0^{17,20}]docosa-1(19),2(22),3,5,14(21),15,17(20)-heptaene (29 mg, 0.06 mmol) and 4N HCl in dioxane (5 mL) was stirred at RT for 16 h. The solvent was evaporated under reduced pressure, co-evaporating with toluene and heptane. The residue was diluted with ethyl acetate and washed with a saturated aqueous NaHCO₃ solution. The organic layer was separated, dried over anhydrous magnesium sulfate, filtered and the solvent was removed under reduced pressure. The residue was purified by silica gel column chromatography using dichloromethane/ethyl acetate 100/0 to 50/50 as eluent. The solid was triturated with diethylether and filtered, yielding 4-(morpholin-4-yl)-7,10-dioxa-13,18,19-triazatetracyclo [12.5.2.1^{2,6}.0^{17,20}]docosa-1(19),2(22),3,5,14(21),15,17(20)-heptaene as a solid.

LCMS method E: [M+H]⁺=381.1, t_{R}=2.38 min.

¹H NMR (300 MHz, d6-DMSO) δ 12.61 (s, 1H), 7.70 (s, 1H), 7.38 (s, 1H), 7.25-7.21 (m, 1H), 6.93 (s, 1H), 6.80-6.77 (d, 1H), 6.40 (s, 1H), 5.77 (bs, 1H), 4.34 (m, 2H), 3.83-3.64 (m, 8H), 3.43-3.36 (m, 2H), 3.13 (m, 2H) ppm.

### Example 2: 7,10,14-trioxa-4,19,20-triazatetracyclo[13.5.2.1^{2,6}.0^{18,21}]tricosa-1(20),2(23), 3,5,15(22),16,18(21)-heptaene

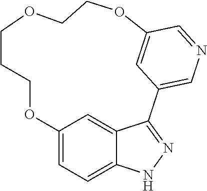 [SMILES: c1cc2[nH]nc3c2cc1OCCCOCCOc1cncc-3c1]

Example 2 is prepared according to the synthesis route described in general Scheme A.

### Preparation of Intermediate 13: 5-((tert-butyldimethylsilyl)oxy)-1H-indazole

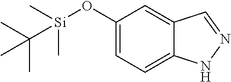 [SMILES: CC(C)(C)[Si](C)(C)Oc1ccc2[nH]ncc2c1]

To a solution of 1H-indazol-5-ol (9.75 g, 72.685 mmol) in DCM (218 mL) was added imidazole (5.94 g, 87.22 mmol) and tert-butylchlorodimethylsilane (12.05 g, 79.95 mmol). The reaction mixture was stirred at RT for 16 h. The reaction was diluted with DCM and washed with a saturated aqueous NaHCO₃ solution then brine. The organic layer was dried over anhydrous magnesium sulfate, filtered and evaporated under reduced pressure. The residue was purified by silica gel column chromatography using heptane/ethyl acetate 100/0 to 70/30 as eluent to afford 5-((tert-butyldimethylsilyl)oxy)-1H-indazole as a yellow oil.

LCMS method B: [M+H]⁺=249.1, t_{R}=1.335 min

### Preparation of Intermediate 14: 5-((tert-butyldimethylsilyl)oxy)-3-iodo-1H-indazole

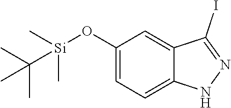 [SMILES: CC(C)(C)[Si](C)(C)Oc1ccc2[nH]nc(I)c2c1]

To a solution of 5-((tert-butyldimethylsilyl)oxy)-3-iodo-1H-indazole (9.29 g, 37.399 mmol) in DCM (112 mL) was added N-iodosuccinimide (9.256 g, 41.139 mmol). The reaction mixture was stirred at RT for 16 h. A saturated aqueous NaHCO₃ solution was added. The two layers were separated and the aqueous layer was extracted with DCM. The combined organic layers were dried over anhydrous magnesium sulfate, filtered and the solvent was removed under reduced pressure. The residue was purified by silica gel column chromatography using heptane/ethyl acetate using 100/0 to 80/20 as eluents to afford 5-((tert-butyldimethylsilyl)oxy)-3-iodo-1H-indazole as a white solid.

LCMS method B: [M+H]⁺=375.0, t_{R}=1.50 min

### Preparation of Intermediate 15: 5-((tert-butyldimethylsilyl)oxy)-3-iodo-1-(tetra hydro-2H-pyran-2-yl)-1H-indazole

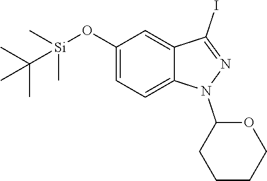 [SMILES: CC(C)(C)[Si](C)(C)Oc1ccc2c(c1)c(I)nn2C1CCCCO1]

To a solution of 5-((tert-butyldimethylsilyl)oxy)-3-iodo-1H-indazole (11.620 g, 30.777 mmol) in DCM (95 mL) was added 3,4-dihydro-2H-pyran (7.767 mL, 92.331 mmol) and 4-methylbenzenesulfonic acid monohydrate (0.585 g, 3.078 mmol). The reaction mixture was stirred at RT for 120 h. A saturated aqueous NaHCO₃ solution was added and the two layers were separated. The aqueous layer was extracted with ethyl acetate. The combined organic layers were dried over anhydrous magnesium sulfate, filtered and evaporated under reduced pressure. The residue was purified by silica gel column chromatography using heptane/ethyl acetate 100/0 to 90/10 as eluent to afford 5-((tert-butyldimethylsilyl)oxy)-3-iodo-1-(tetrahydro-2H-pyran-2-yl)-1H-indazole as a red solid.

LCMS method B: [M+H]⁺=459.1, t_{R}=1.528 min

### Preparation of Intermediate 16: 3-iodo-1-(tetrahydro-pyran-2-yl)-1H-indazol-5-ol

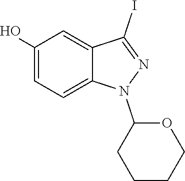 [SMILES: Oc1ccc2c(c1)c(I)nn2C1CCCCO1]

To a solution of 5-((tert-butyldimethylsilyl)oxy)-3-iodo-1-(tetrahydro-2H-pyran-2-yl)-1H-indazole (12.27 g, 26.76 mmol) in THF (80 mL) was added at 0° C. TBAF (1M solution in THF) (40.15 mL, 40.15 mmol). The reaction mixture was stirred at RT for 1 h. The mixture was diluted with ethyl acetate, washed with a saturated aqeuous NaHCO₃ solution. The organic layer was dried over anhydrous magnesium sulfate, filtered and evaporated under reduced pressure. The residue was purified by silica gel column chromatography using heptane/ethyl acetate 100/0 to 70/30 as eluent to afford 3-iodo-1-(tetrahydro-pyran-2-yl)-1H-indazol-5-ol as a white solid.

LCMS method B: [M+H]⁺=345.0, t_{R}=0.766 min

### Preparation of Intermediate 17: tert-butyl-[3-(3-iodo-1-tetrahydropyran-2-yl-indazol-5-yl)oxypropoxy]-dimethyl-silane

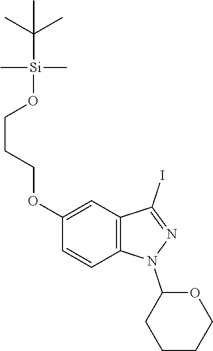 [SMILES: CC(C)(C)[Si](C)(C)OCCCOc1ccc2c(c1)c(I)nn2C1CCCCO1]

To a stirred to solution of 3-iodo-1-(tetrahydro-2H-pyran-2-yl)-1H-indazol-5-ol (2.47 g, 7.177 mmol) in DMA (22 mL) was added cesium carbonate (4.677 g, 14.354 mmol). The reaction mixture was stirred at RT for 30 min, then (3-bromopropoxy)(tert-butyl)dimethylsilane (1.834 mL, 7.895 mmol) was added. The reaction mixture was stirred at RT for 16 h. The mixture was diluted with ethyl acetate, washed with water and extracted with ethyl acetate. The combined organic layers were dried over anhydrous magnesium sulfate, filtered and evaporated under reduced pressure. The residue was purified by silica gel column chromatography using heptane/ethyl acetate 100/0 to 85/15 as eluent to afford tert-butyl-[3-(3-iodo-1-tetrahydropyran-2-yl-indazol-5-yl)oxypropoxy]-dimethyl-silane as a colourless oil.

LCMS method B: [M+H]⁺=517.2, t_{R}=1.568 min.

### Preparation of Intermediate 18: 3-(3-iodo-1-tetrahydropyran-2-yl-indazol-5-yl) oxypropan-1-ol

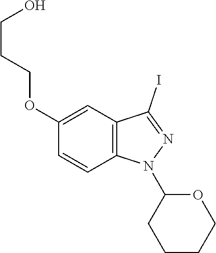 [SMILES: OCCCOc1ccc2c(c1)c(I)nn2C1CCCCO1]

To a solution of tert-butyl-[3-(3-iodo-1-tetrahydropyran-2-yl-indazol-5-yl)oxypropoxy]-dimethyl-silane (3.6 g, 6.97 mmol) in THF (21 mL) was added TBAF (mL solution in THF) (10.5 mL, 10.5 mmol) at 0° C. The reaction mixture was stirred at RT for 16 h. The mixture was washed with a saturated aqeous NaHCO₃ solution and extracted with ethyl acetate. The combined organic layers were dried over anhydrous magnesium sulfate, filtered and evaporated under reduced pressure. The residue was purified by silica gel column chromatography using heptane/ethyl acetate 100/0 to 50/50 as eluent to afford 3-(3-iodo-1-tetrahydropyran-2-yl-indazol-5-yl)oxypropan-1-ol as a beige sticky solid.

LCMS method A: [M+H]⁺=402.9, t_{R}=0.765 min

### Preparation of Intermediate 19: tert-butyl 2-[3-(3-iodo-1-tetrahydropyran-2-yl-indazol-5-yl)oxypropoxy]acetate

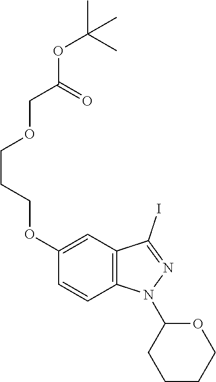 [SMILES: CC(C)(C)OC(=O)COCCCOc1ccc2c(c1)c(I)nn2C1CCCCO1]

A mixture of 3-(3-iodo-1-tetrahydropyran-2-yl-indazol-5-yl)oxypropan-1-ol (2.0 g, 4.972 mmol), tert-butyl 2-bromoacetate (1.94 mL, 9.944 mmol) and tetrabutylammonium hydrogen sulfate (169 mg, 0.497 mmol) was dissolved in toluene (16 mL) and aqueous sodium hydroxide solution (50% w/w) (16 mL). The reaction mixture was stirred at RT for 16 h. The reaction mixture was diluted with ethyl acetate and acidified with hydrochloric acid (37% v/v) until pH 6. Water was added and the mixture was extracted with ethyl acetate. The combined organic layers were dried over anhydrous magnesium sulfate, filtered and evaporated under reduced pressure. The residue was purified by silica gel column chromatography using heptane/ethyl acetate 100/0 to 80/20 as eluent to afford tert-butyl 2-[3-(3-iodo-1-tetrahydropyran-2-yl-indazol-5-yl)oxypropoxy]acetate as a yellowish oil.

LCMS method B: [M+H]⁺=517.0, t_{R}=1.236 min.

### Preparation of Intermediate 22: tert-butyl 2-[3-[3-(5-benzyloxy-3-pyridyl)-1-tetrahydropyran-2-yl-indazol-5-yl]oxypropoxy]acetate

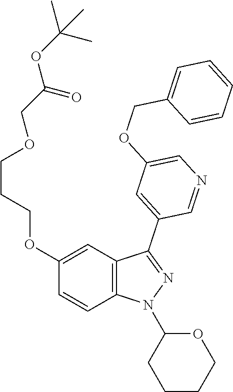 [SMILES: CC(C)(C)OC(=O)COCCCOc1ccc2c(c1)c(-c1cncc(OCc3ccccc3)c1)nn2C1CCCCO1]

To a mixture of tert-butyl 2-(3-((3-iodo-1-(tetrahydro-2H-pyran-2-yl)-1H-indazol-5-yl)oxy) propoxy)acetate (2.5 g, 4.841 mmol), (5-(benzyloxy)pyridin-3-yl)boronic acid (1.663 g, 7.261 mmol) and potassium phosphate tribasic (3.08 g, 14.523 mmol) in dioxane (36 mL) and water (12 mL) was added tetrakis(triphenylphosphine)palladium(0) (280 mg, 0.242 mmol) and XPhos (231 mg, 0.484 mmol). The reaction mixture was degassed with N₂ for 5 min and stirred at 90° C. for 16 h. The reaction mixture was quenched with a saturated aqueous NaHCO₃ solution and extracted with ethyl acetate. The combined organic layers were dried over anhydrous magnesium sulfate, filtered and evaporated under reduced pressure. The residue was purified by silica gel column chromatography using heptane/ethyl acetate 100/0 to 50/50 as eluent to afford tert-butyl2-[3-[3-(5-benzyloxy-3-pyridyl)-1-tetrahydropyran-2-yl-indazol-5-yl]oxypropoxy] acetate as yellow oil.

LCMS method B: [M+H]⁺=574.3, t_{R}=1.318 min.

### Preparation of Intermediate 23: 2-[3-[3-(5-benzyloxy-3-pyridyl)-1-tetrahydropyran-2-yl-indazol-5-yl]oxypropoxy]ethanol

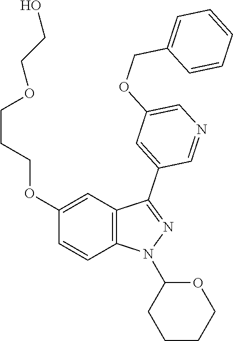 [SMILES: OCCOCCCOc1ccc2c(c1)c(-c1cncc(OCc3ccccc3)c1)nn2C1CCCCO1]

To a solution of tert-butyl 2-[3-[3-(5-benzyloxy-3-pyridyl)-1-tetrahydropyran-2-yl-indazol-5-yl]oxypropoxy]acetate (1.023 g, 1.783 mmol) in dry THF (69 mL) at 0° C. was added lithium aluminum hydride (677 mg, 17.83 mmol). The reaction mixture was stirred at RT for 3 h. The reaction mixture was quenched with 1M aqueous sodium hydroxide solution and extracted with ethyl acetate. The combined organic layers were dried over anhydrous magnesium sulfate, filtered and evaporated under reduced pressure. The residue was purified by silica gel column chromatography using dichloromethane/methanol 100/0 to 97/3 as eluent to afford 2-[3-[3-(5-benzyloxy-3-pyridyl)-1-tetrahydropyran-2-yl-indazol-5-yl]oxypropoxy]ethanol as a yellow oil.

LCMS method B: [M+H]⁺=504.2, t_{R}=1.05 min.

### Preparation of Intermediate 24: 5-[5-[3-(2-hydroxyethoxy)propoxy]-1-tetrahydro pyran-2-yl-indazol-3-yl]pyridin-3-ol

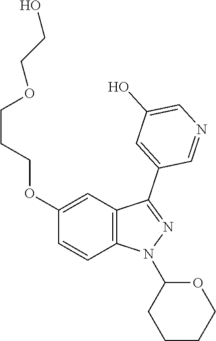 [SMILES: OCCOCCCOc1ccc2c(c1)c(-c1cncc(O)c1)nn2C1CCCCO1]

To a solution of 2-[3-[3-(5-benzyloxy-3-pyridyl)-1-tetrahydropyran-2-yl-indazol-5-yl]oxy propoxy]ethanol (206 mg, 0.409 mmol) in ethanol (6 mL) was added palladium on activated carbon 10% (80 mg). The reaction mixture was stirred under hydrogen atmosphere at RT for 1 h. The reaction mixture was filtered through a pad of celite rinsing with ethyl acetate and the solvent was evaporated under reduced pressure. The residue was purified by silica gel column chromatography using dichloromethane/(methanol/ammonia) 100/0 to 90/10 as eluent to afford 5-[5-[3-(2-hydroxyethoxy)propoxy]-1-tetrahydropyran-2-yl-indazol-3-yl]pyridin-3-ol as white solid.

LCMS method B: [M+H]⁺=414.2, t_{R}=0.612 min.

### Preparation of Intermediate 25: 5-[5-[3-(2-chloroethoxy)propoxy]-1-tetrahydro pyran-2-yl-indazol-3-yl]pyridin-3-ol

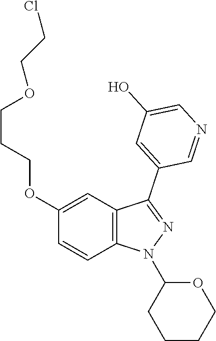 [SMILES: Oc1cncc(-c2nn(C3CCCCO3)c3ccc(OCCCOCCCl)cc23)c1]

To a solution of 5-[5-[3-(2-hydroxyethoxy)propoxy]-1-tetrahydropyran-2-yl-indazol-3-yl]pyridin-3-ol (75 mg, 0.181 mmol) and pyridine (0.073 mL, 0.905 mmol) in dry DCM (1.6 mL) at 0° C. was added thionyl chloride (0.066 mL, 0.905 mmol). The reaction mixture was stirred at RT for 24 h. The reaction was quenched with a 1M aqueous NaHCO₃ solution and extracted with ethyl acetate. The organic layer was dried over anhydrous magnesium sulfate, filtered and evaporated under reduced pressure. The residue was purified by silica gel column chromatography using dichloromethane/methanol 100/0 to 90/10 as eluent to afford 5-[5-[3-(2-chloroethoxy)propoxy]-1-tetrahydropyran-2-yl-indazol-3-yl]pyridin-3-ol as a colourless oil.

LCMS method B: [M+H]⁺=432.1-434.1, t_{R}=0.836 min.

### Preparation of Intermediate 26: 19-(oxan-2-yl)-7,10,14-trioxa-4,19,20-triazatetra cyclo[13.5.2.1^{2,6}.0^{18,21}]tricosa-1(20),2(23),3,5,15(22),16,18(21)-heptaene

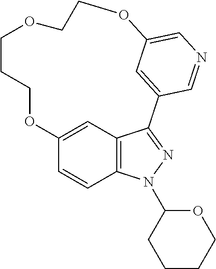 [SMILES: c1cc2c3cc1OCCCOCCOc1cncc(c1)-c3nn2C1CCCCO1]

To a solution of 5-[5-[3-(2-chloroethoxy)propoxy]-1-tetrahydropyran-2-yl-indazol-3-yl] pyridin-3-ol (33 mg, 0.076 mmol) in dry DMA (10 mL) was added dropwise a suspension of cesium carbonate (74 mg, 0.228 mmol) in dry DMA (5 mL) at RT under nitrogen atmosphere. The reaction mixture was stirred at 90° C. for 16 h. A saturated aqueous NaHCO₃ solution was added and the mixture was extracted with ethyl acetate. The combined organic phases were dried over anhydrous magnesium sulfate, filtered and evaporated under reduced pressure. The residue was purified by silica gel column chromatography heptane/ethyl acetate 100/0 to 20/80 as eluent to afford 19-(oxan-2-yl)-7,10,14-trioxa-4,19,20-triazatetracyclo[13.5.2.1^{2,6}.0^{18,21}] tricosa-1(20),2(23),3,5,15,17,21-heptaene as a yellow oil.

LCMS method B: [M+H]⁺=396.2, t_{R}=0.974 min.

### Preparation of Example 2: 7,10,14-trioxa-4,19,20-triazatetracyclo[13.5.2.1^{2,6}.0^{18,21}] tricosa-1(20),2(23),3,5,15(22),16,18(21)-heptaene

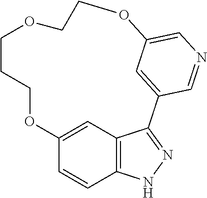 [SMILES: c1cc2[nH]nc3c2cc1OCCCOCCOc1cncc-3c1]

A solution of 19-(oxan-2-yl)-7,10,14-trioxa-4,19,20-triazatetracyclo[13.5.2.1^{2,6}.0^{18,21}]tricosa-1(20),2(23),3,5,15,17,21-heptaene (16 mg, 0.04 mmol) in HCl (4M solution in dioxane) (6 mL) was stirred at RT for 16 h. The solvent was removed under reduced pressure. A saturated aqueous NaHCO₃ solution was added and the mixture was extracted with ethyl acetate. The combined organic layers were dried over anhydrous magnesium sulfate, filtered and evaporated under reduced pressure. The residue was purified by silica gel column chromatography using dichloromethane/methanol 100/0 to 90/10 as eluent to afford 7,10,14-trioxa-4,19,20-triazatetracyclo[13.5.2.1^{2,6}.0^{18,21}]tricosa-1(20),2(23),3,5,15,17,22-heptaene as a solid.

LCMS method E: [M+H]⁺=312.1, t_{R}=2.375 min.

LCMS method D: [M+H]⁺=312.2, t_{R}=4.164 min.

¹H NMR (300 MHz, d6-DMSO) δ 13.22 (s, 1H), 8.74 (s, 1H), 8.40 (s, 1H), 8.20 (s, 1H), 7.93 (s, 1H), 7.51 (d, J=8.9 Hz, 1H), 7.01 (d, J=8.5 Hz, 1H), 4.54 (s, 2H), 4.40 (s, 2H), 3.83 (s, 2H), 3.55 (s, 2H), 2.07 (brs, 2H) ppm.

### Example 3: 4-(morpholin-4-yl)-7,11,14-trioxa-19,20-diazatetracyclo[13.5.2.1^{2,6}.0^{18,21}] tricosa-1(20),2(23),3,5,15(22),16,18(21)-heptaene

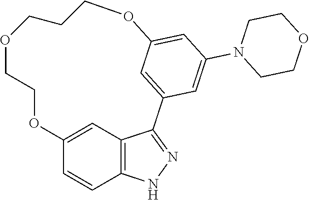 [SMILES: c1cc2[nH]nc3c2cc1OCCOCCCOc1cc-3cc(N2CCOCC2)c1]

Example 3 is prepared according to the synthesis route described in general Scheme A.

### Preparation of Intermediate 27: 2-(3-benzyloxypropoxy)ethanol

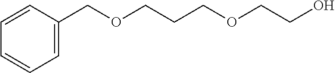 [SMILES: OCCOCCCOCc1ccccc1]

To a solution of ethylene glycol (5.57 mL, 100 mmol) in DMF (30 mL) at 0° C. was added portionwise sodium hydride (60% dispersion in mineral oil) (2.0 g, 50 mmol). The suspension was stirred at 0° C. for 1 h and 3-bromopropoxymethylbenzene (1.76 mL, 10 mmol) was added dropwise. The reaction mixture was warmed to RT and stirred overnight. Water was added to the reaction mixture and extracted with ethyl acetate. The combined organic layers were washed with brine, dried over anhydrous sodium sulfate, filtered and evaporated under reduced pressure. The residue was purified by silica gel column chromatography using dichloro methane/methanol 98/2 as eluent to afford 2-(3-benzyloxypropoxy)ethanol as a colorless oil.

LCMS method F: [M+H]⁺=211.2, t_{R}=2.06 min.

### Preparation of Intermediate 28: 2-(3-benzyloxypropoxy)ethyl methanesulfonate

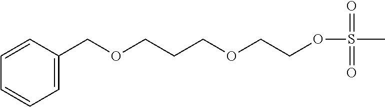 [SMILES: CS(=O)(=O)OCCOCCCOCc1ccccc1]

To a cooled solution of 2-(3-benzyloxypropoxy)ethanol (1.1 g, 5.23 mmol) and DIPEA (1.82 mL, 10.46 mmol) in DCM (20 mL) was added dropwise methanesulfonyl chloride (485 μL, 6.27 mmol). The reaction mixture was stirred at RT for 3 h. The organic phase was washed with a saturated aqueous solution of ammonium chloride, saturated aqueous NaHCO₃ solution and brine. The organic phase was dried over anhydrous sodium sulfate, filtered and evaporated under reduced pressure to afford 2-(3-benzyloxypropoxy)ethyl methanesulfonate as an orange oil which was used in the next step without further purification.

LCMS method F: [M+H]⁺=289.1, t_{R}=2.46 min.

### Preparation of Intermediate 29: 5-[2-(3-benzyloxypropoxy)ethoxy]-3-iodo-1-tetra hydropyran-2-yl-indazole

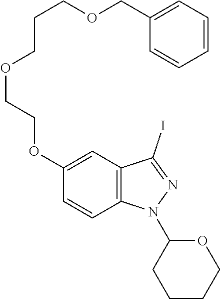 [SMILES: Ic1nn(C2CCCCO2)c2ccc(OCCOCCCOCc3ccccc3)cc12]

To a solution of 3-iodo-1-tetrahydropyran-2-yl-indazol-5-ol (1.51 g, 4.58 mmol) and cesium carbonate (4.28 g, 13.16 mmol) in DMF (15 mL) was added dropwise a solution of tert-butyl 3-(methylsulfonyloxymethyl)pyrrolidine-1-carboxylate (1.39 g, 4.82 mmol) in DMF (5 mL). The reaction mixture was stirred at 60° C. overnight. The reaction mixture was cooled to RT and water was added. The aqueous phase was extracted with ethyl acetate and the combined organic layers were washed with brine, dried over anhydrous sodium sulfate, filtered and evaporated under reduced pressure to afford 5-[2-(3-benzyloxypropoxy)ethoxy]-3-iodo-1-tetrahydro pyran-2-yl-indazole as an orange oil which was used in the next step without further purification.

LCMS method F: [M+H]⁺=537.1, t_{R}=3.40 min.

### Preparation of Intermediate 30: 3-chloro-5-morpholino-phenol

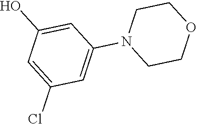 [SMILES: Oc1cc(Cl)cc(N2CCOCC2)c1]

To a degassed solution of 3-bromo-5-chlorophenol (6.21 g, 30 mmol), morpholine (2.89 mL, 33 mmol), tBuONa (5.76 g, 60 mmol) and SPhos (490 mg, 1.2 mmol) in dioxane (40 mL) was added Pd₂(dba)₃ (550 mg, 0.6 mmol). The reaction mixture was stirred at 80° C. for 2 h. After being cooled to RT, the reaction mixture was diluted with water and extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent to afford 3-chloro-5-morpholino-phenol as a yellow solid.

LCMS method F: [M+H]⁺=214.1, t_{R}=2.10 min.

### Preparation of Intermediate 31: 3-(5,5-dimethyl-1,3,2-dioxaborinan-2-yl)-5-morpholino-phenol

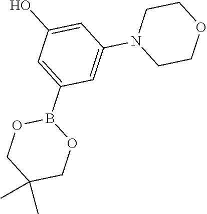 [SMILES: CC1(C)COB(c2cc(O)cc(N3CCOCC3)c2)OC1]

To a degassed solution of 3-chloro-5-morpholino-phenol (3.5 g, 16.43 mmol), bis(neopentyl glycolato)diboron (5.57 g, 24.65 mmol), potassium acetate (3.22 g, 32.86 mmol) and XPhos (314 mg, 0.66 mmol) in dioxane (40 mL) was added XPhos-Pd G2 (260 mg, 0.33 mmol) at RT. The reaction mixture was stirred for 3 h at 80° C. After being cooled to RT, the reaction mixture was concentrated under reduced pressure then diluted with 1N aqueous NaOH solution and extracted with ethyl acetate. The combined aqueous layers were neutralized with 1N HCl and extracted with ethyl acetate. The combined organic layers were dried over anhydrous sodium sulfate. The residue was purified by silica gel column chromatography using cyclohexane/ethyl acetate 100/0 to 40/60 as eluent to afford 3-(5,5-dimethyl-1,3,2-dioxaborinan-2-yl)-5-morpholino-phenol as a white solid.

LCMS method H: [M+H]⁺=224.1, t_{R}=1.55 min (corresponding to boronic acid mass).

### Preparation of Intermediate 32: 3-[5-[2-(3-benzyloxypropoxy)ethoxy]-1-tetrahydro pyran-2-yl-indazol-3-yl]-5-morpholino-phenol

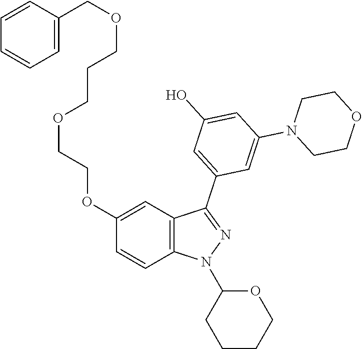 [SMILES: Oc1cc(-c2nn(C3CCCCO3)c3ccc(OCCOCCCOCc4ccccc4)cc23)cc(N2CCOCC2)c1]

To a solution of 5-[2-(3-benzyloxypropoxy)ethoxy]-3-iodo-1-tetrahydropyran-2-yl-indazole (500 mg, 0.93 mmol), 3-(5,5-dimethyl-1,3,2-dioxaborinan-2-yl)-5-morpholino-phenol (271 mg, 0.93 mmol) and Na₂CO₃ (296 mg, 2.79 mmol) in DME (10 mL) and water (1 mL) was added tetrakis(triphenylphosphine)-palladium(0) (54 mg, 0.046 mmol). The reaction mixture was stirred at 80° C. overnight. After being cooled to RT, the reaction mixture was diluted with water and extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 70/30 to 50/50 as eluent to afford 3-[5-[2-(3-benzyloxypropoxy)ethoxy]-1-tetrahydropyran-2-yl-indazol-3-yl]-5-morpholino-phenol as a yellow oil.

LCMS method F: [M+H]⁺=588.3, t_{R}=3.06 min.

### Preparation of Intermediate 33: 3-[5-[2-(3-hydroxypropoxy)ethoxy]-1-tetrahydro pyran-2-yl-indazol-3-yl]-5-morpholino-phenol

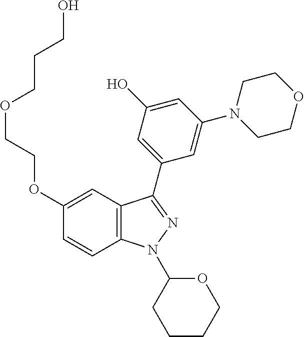 [SMILES: OCCCOCCOc1ccc2c(c1)c(-c1cc(O)cc(N3CCOCC3)c1)nn2C1CCCCO1]

To a mixture of 3-[5-[2-(3-benzyloxypropoxy)ethoxy]-1-tetrahydropyran-2-yl-indazol-3-yl]-5-morpholino-phenol (250 mg, 0.43 mmol) in methanol (2 mL) and ethyl acetate (2 mL) was added Pd/C 10% (23 mg, 0.21 mmol). The reaction mixture was stirred at RT overnight under hydrogen atmosphere. The palladium was filtered off and the solvent was removed under reduced pressure to afford 3-[5-[2-(3-hydroxypropoxy)ethoxy]-1-tetrahydropyran-2-yl-indazol-3-yl]-5-morpholino-phenol as colourless oil. The product was used in the next step without further purification.

LCMS method F: [M+H]⁺=498.3, t_{R}=2.27 min

### Preparation of Intermediate 34: 4-(morpholin-4-yl)-19-(oxan-2-yl)-7,11,14-trioxa-19,20-diazatetracyclo[13.5.2.1^{2,6}.0^{18,21}]tricosa-1(20),2(23),3,5,15(22),16,18(21)-heptaene

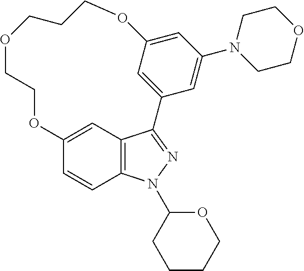 [SMILES: c1cc2c3cc1OCCOCCCOc1cc(cc(N4CCOCC4)c1)-c3nn2C1CCCCO1]

To a mixture of triphenylphosphine (237 mg, 0.9 mmol) in toluene (50 mL) at 90° C. was added simultaneously DIAD (178 μL, 0.9 mmol) in MeTHF (15 mL) and 3-[5-[2-(3-hydroxypropoxy) ethoxy]-1-tetrahydropyran-2-yl-indazol-3-yl]-5-morpholino-phenol (150 mg, 0.3 mmol) in MeTHF (15 mL) over a period of 10 min. The reaction mixture was stirred at 90° C. overnight. The solvent was evaporated under reduced pressure. The residue was purified by silica gel column chromatography using cyclohexane/ethyl acetate 100/0 to 70/30 as eluent to afford 4-(morpholin-4-yl)-19-(oxan-2-yl)-7,11,14-trioxa-19,20-diazatetracyclo[13.5.2.1^{2,6}.0^{18,21}] tricosa-1(20),2(23),3,5,15(22),16,18(21)-heptaene as a colourless oil.

LCMS method F: [M+H]⁺=480.3, t_{R}=3.12 min

### Preparation of Example 3: 4-(morpholin-4-yl)-7,11,14-trioxa-19,20-diazatetracyclo [13.5.2.1^{2,6}.0^{18,21}]tricosa-1(20),2(23),3,5,15(22),16,18(21)-heptaene

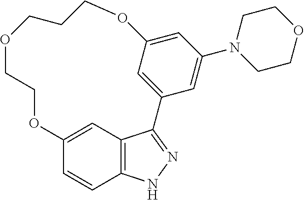 [SMILES: c1cc2[nH]nc3c2cc1OCCOCCCOc1cc-3cc(N2CCOCC2)c1]

To a mixture of 4-(morpholin-4-yl)-19-(oxan-2-yl)-7,11,14-trioxa-19,20-diazatetracyclo [13.5.2.1^{2,6}.0^{18,21}]tricosa-1(20),2(23),3,5,15(22),16,18(21)-heptaene (66 mg, 0.14 mmol) in dioxane (3 mL) was added HCl (4 M solution in dioxane) (345 μL; 1.38 mmol). The reaction mixture was stirred at RT for 2 h. The solid was filtered and washed with diisopropylether, dried under reduced pressure to afford 4-(morpholin-4-yl)-7,11,14-trioxa-19,20-diazatetracyclo [13.5.2.1^{2,6}.0^{18,21}]tricosa-1(20),2(23),3,5,15(22),16,18(21)-heptaene as a powder.

LCMS method F: [M+H]⁺=396.2, t_{R}=2.50 min

LCMS method G: [M+H]⁺=396.3, t_{R}=2.53 min

¹H NMR (400 MHz, d6-DMSO) δ 8.46 (1H, d, J=2.1 Hz), 7.45-7.38 (2H, m), 7.29 (1H, d, J=1.3 Hz), 7.02 (1H, dd, J=2.3, 8.9 Hz), 6.43 (1H, t, J=2.1 Hz), 4.47-4.37 (4H, m), 3.81-3.74 (6H, m), 3.6 (2H, m), 3.18-3.14 (4H, m), 2.11-2.05 (2H, m) ppm.

### Example 4: 7,10-dioxa-4,14,19,20-tetraazatetracyclo[13.5.2.1^{2,6}.0^{18,21}]tricosa-1(20),2(23), 3,5,15(22),16,18(21)-heptaene

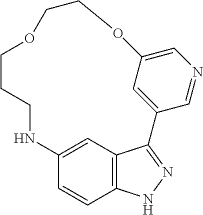 [SMILES: c1cc2[nH]nc3c2cc1NCCCOCCOc1cncc-3c1]

Example 4 is prepared according to the synthesis route described in general Scheme A.

### Preparation of Intermediate 35: tert-butyl-2-[3-[tert-butyl(dimethyl)silyl]oxypropoxy] acetate

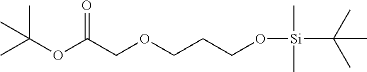 [SMILES: CC(C)(C)OC(=O)COCCCO[Si](C)(C)C(C)(C)C]

A mixture of 3-((tert-butyldimethylsilyl)oxy)propan-1-ol (3.0 g, 15.76 mmol), tert-butyl 2-bromoacetate (6.148 mL, 31.52 mmol) and tetrabutylammonium hydrogen sulfate (535 mg, 1.576 mmol) in toluene (51 mL) and aqueous NaOH solution (50% w/w) (51 mL) was stirred at RT for 16 h. Ethyl acetate was added and the mixture was washed with a saturated aqueous NaHCO₃ solution. The aqueous phase was extracted with ethyl acetate. The combined organic layers were dried over anhydrous magnesium sulfate, filtered and evaporated under reduced pressure. The residue was purified by silica gel column chromatography using heptane/ethyl acetate 100/0 to 80/20 as eluent to afford tert-butyl-2-[3-[tert-butyl(dimethyl)silyl] oxypropoxy]acetate as a colourless oil.

LCMS method A: [M+Na]⁺=327.0, t_{R}=1.244 min.

### Preparation of Intermediate 36: tert-butyl 2-(3-hydroxypropoxy)acetate

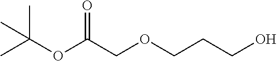

To a solution of tert-butyl 2-(3-((tert-butyldimethylsilyl)oxy)propoxy)acetate (4.77 g, 15.76 mmol) in THF (47 mL) at 0° C. was added TBAF (1M solution in THF) (23.64 mL, 23.64 mmol). The reaction mixture was stirred at RT for 16 h. A saturated aqeous NaHCO₃ solution was added and the mixture was extracted with ethyl acetate. The combined organic layers were dried over anhydrous magnesium sulfate, filtered and evaporated under reduced pressure. The residue was purified by silica gel column chromatography using heptane/ethyl acetate 100/0 to 50/50 as eluent. The desired fractions were combined and the solvent was removed under reduced pressure yielding tert-butyl 2-(3-hydroxypropoxy)acetate as a yellowish oil.

LCMS method A: [M+Na]⁺=213.0, t_{R}=0.444 min.

### Preparation of Intermediate 37: tert-butyl 2-[3-[(3-iodo-1-tetrahydropyran-2-yl-indazol-5-yl)-(2-nitrophenyl)sulfonyl-amino]propoxy]acetate

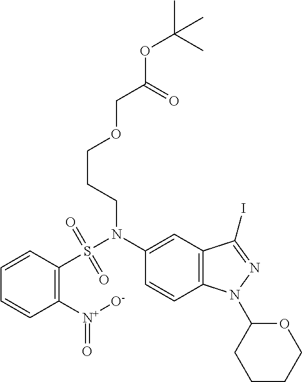 [SMILES: CC(C)(C)OC(=O)COCCCN(c1ccc2c(c1)c(I)nn2C1CCCCO1)S(=O)(=O)c1ccccc1[N+](=O)[O-]]

To a solution of N-(3-iodo-1-tetrahydropyran-2-yl-indazol-5-yl)-2-nitro-benzenesulfonamide (1.10 g, 2.082 mmol) in dry THF (15 mL) under N₂ atmosphere was added tert-butyl 2-(3-hydroxypropoxy)acetate (0.792 g, 4.164 mmol) and triphenylphosphine (1.092 g, 4.164 mmol). The solution was cooled to 0° C. and diisopropyl azodicarboxylate (0.8 mL, 4.075 mmol) was added dropwise. The solution was allowed to warm up to RT, heated to 90° C. and stirred for 4 h. The solvent was removed under reduced pressure and the residue was purified by silica gel column chromatography using dichloromethane/methanol 100/0 to 90/10 as eluent to afford tert-butyl-2-[3-[(3-iodo-1-tetrahydropyran-2-yl-indazol-5-yl)-(2-nitrophenyl)sulfonyl-amino] propoxy]acetate as an orange oil.

LCMS method B: [M+Na]⁺=723.2, t_{R}=1.258 min.

### Preparation of Intermediate 38: tert-butyl 2-[3-[[3-(5-hydroxy-3-pyridyl)-1-tetrahydro pyran-2-yl-indazol-5-yl]-(2-nitrophenyl)sulfonyl-amino]propoxy]acetate

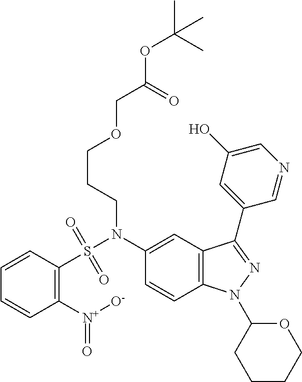 [SMILES: CC(C)(C)OC(=O)COCCCN(c1ccc2c(c1)c(-c1cncc(O)c1)nn2C1CCCCO1)S(=O)(=O)c1ccccc1[N+](=O)[O-]]

To a mixture of tert-butyl 2-[3-[(3-iodo-1-tetrahydropyran-2-yl-indazol-5-yl)-(2-nitrophenyl) sulfonylamino]propoxy]acetate (151 mg, 0.216 mmol), 5-(4,4,5,5-tetramethyl-1,3,2-dioxa borolan-2-yl)pyridin-3-ol (55 mg, 0.248 mmol) and potassium phosphate tribasic (138 mg, 0.648 mmol) in dioxane/water (3:1) (11 mL) was added Pd(PPh₃)₄ (13 mg, 0.011 mmol) and XPhos (10 mg, 0.022 mmol), degassed with nitrogen for 5 min and was heated at 100° C. for 1 h under microwave irradiations. Additional Pd(PPh₃)₄ (13 mg, 0.011 mmol), XPhos (10 mg, 0.022 mmol), 5-(4,4,5,5-tetramethyl-1,3,2-dioxaborolan-2-yl)pyridin-3-ol (55 mg, 0.248 mmol) and potassium phosphate tribasic (138 mg, 0.648 mmol) were added, degassed with nitrogen for 5 min and heated to 100° C. for 1 h under microwave irradiations. Water was added and the mixture was extracted with ethyl acetate. The combined organic layers were dried over anhydrous magnesium sulfate and evaporated under reduced pressure. The residue was purified by silica gel column chromatography using dichloromethane/methanol 100/0 to 80/20 as eluent to afford tert-butyl-2-[3-[[3-(5-hydroxy-3-pyridyl)-1-tetrahydropyran-2-yl-indazol-5-yl]-(2-nitrophenyl) sulfonyl-amino]propoxy]acetate.

LCMS method B: [M+H]⁺=668.2, t_{R}=1.07 min.

### Preparation of Intermediate 39: 2-(3-((N-(3-(5-hydroxypyridin-3-yl)-1H-indazol-5-yl)-2-nitrophenyl)sulfonamido)propoxy)acetic acid hydrochloride

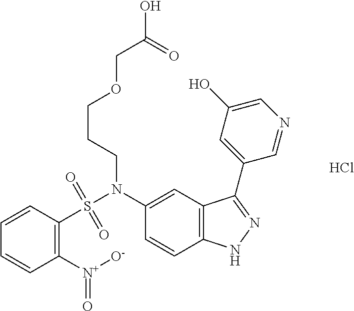 [SMILES: O=C(O)COCCCN(c1ccc2[nH]nc(-c3cncc(O)c3)c2c1)S(=O)(=O)c1ccccc1[N+](=O)[O-] ; Cl]

A mixture of tert-butyl 2-[3-[[3-(5-hydroxy-3-pyridyl)-1-tetrahydropyran-2-yl-indazol-5-yl]-(2-nitrophenyl)sulfonyl-amino]propoxy]acetate (161 mg, 0.241 mmol) and HCl (4M solution in dioxane) (2.5 mL) was stirred at RT for 16 h. The solvent was removed under reduced pressure. The residue was successively diluted with toluene and heptane and the solvents were removed under reduced pressure to yield 2-(3-((N-(3-(5-hydroxypyridin-3-yl)-1H-indazol-5-yl)-2-nitrophenyl)sulfonamido)propoxy)acetic acid hydrochloride as an orange foam which was used in the next step without further purification.

LCMS method B: [M+H]⁺=528.2, t_{R}=0.50 min.

### Preparation of Intermediate 40: N-[3-(2-hydroxyethoxy)propyl]-N-[3-(5-hydroxy-3-pyridyl)-1H-indazol-5-yl]-2-nitro-benzenesulfonamide

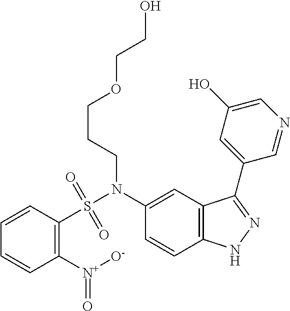 [SMILES: O=[N+]([O-])c1ccccc1S(=O)(=O)N(CCCOCCO)c1ccc2[nH]nc(-c3cncc(O)c3)c2c1]

A mixture of 2-(3-((N-(3-(5-hydroxypyridin-3-yl)-1H-indazol-5-yl)-2-nitrophenyl) sulfonamido)propoxy)acetic acid hydrochloride (129 mg, 0.309 mmol) in borane dimethyl sulfide complex (2M solution in THF) (1.5 mL) was stirred at RT for 18 h. The mixture was cooled to 0° C. and quenched with methanol. The reaction mixture was concentrated under reduced pressure and co-evaporated twice with heptane. HCl (4M solution in dioxane) (5 mL) was added to the residue and the reaction mixture was stirred at RT for 16 h. The reaction mixture was concentrated under reduced pressure and co-evaporated with heptane. Ethyl acetate was added and the mixture was neutralized with a saturated aqueous NaHCO₃ solution. The mixture was extracted with ethyl acetate. The combined organic layers were dried over anhydrous magnesium sulfate, filtered and evaporated under reduced pressure. The residue was purified by silica gel column chromatography using dichloromethane/methanol 100/0 to 93/7 as eluent to afford N-(3-(2-hydroxyethoxy)propyl)-N-(3-(5-hydroxypyridin-3-yl)-1H-indazol-5-yl)-2-nitrobenzenesulfonamide as a colourless oil.

LCMS method B: [M+H]⁺=514.1, t_{R}=0.509 min.

### Preparation of Intermediate 41: N-[3-(2-chloroethoxy)propyl]-N-[3-(5-hydroxy-3-pyridyl)-1H-indazol-5-yl]-2-nitro-benzenesulfonamide

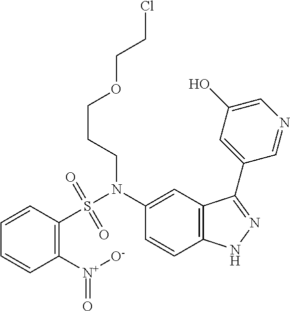 [SMILES: O=[N+]([O-])c1ccccc1S(=O)(=O)N(CCCOCCCl)c1ccc2[nH]nc(-c3cncc(O)c3)c2c1]

In a sealed tube, a mixture of N-(3-(2-hydroxyethoxy)propyl)-N-(3-(5-hydroxypyridin-3-yl)-1H-indazol-5-yl)-2-nitrobenzenesulfonamide (45 mg, 0.087 mmol), thionyl chloride (26 □L, 0.352 mmol) in CHCl₃ (0.9 mL) was heated at 80° C. for 3 h. The reaction mixture was diluted with dichloromethane, cooled to 0° C. and quenched with a saturated aqueous NaHCO₃ solution.

The mixture was extracted with dichloromethane. The combined organic layers were dried over anhydrous magnesium sulfate, filtered and evaporated under reduced pressure. The residue was purified by silica gel column chromatography using dichloromethane/methanol 100/0 to 95/5 as eluent to afford N-[3-(2-chloroethoxy)propyl]-N-[3-(5-hydroxy-3-pyridyl)-1H-indazol-5-yl]-2-nitro-benzenesulfonamide as a colourless foam.

LCMS method A: [M+H]⁺=531.9-533.9, t_{R}=0.673 min.

### Preparation of Intermediate 42: 14-(2-nitrobenzenesulfonyl)-7,10-dioxa-4,14,19,20-tetraazatetracyclo[13.5.2.1^{2,6}.0^{18,21}]tricosa-1(20),2(23),3,5,15(22),16,18(21)-heptaene

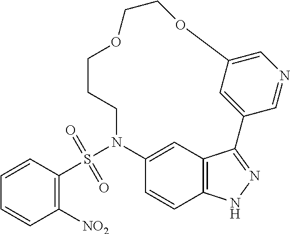 [SMILES: O=[N+]([O-])c1ccccc1S(=O)(=O)N1CCCOCCOc2cncc(c2)-c2n[nH]c3ccc1cc23]

N-[3-(2-Chloro-ethoxy)-propyl]-N-[3-(5-hydroxy-pyridin-3-yl)-1H-indazol-5-yl]-2-nitro-benzenesulfonamide (24 mg, 0.046 mmol) in dry DMA (7 mL) at 90° C. was added dropwise to a stirred suspension of cesium carbonate (73 mg, 0.225 mmol) in dry DMA (2 mL) at 90° C. The reaction mixture was stirred at 90° C. for 4 h. The mixture was diluted with ethyl acetate and water. The aqeous layer was extracted with ethyl acetate. The combined organic layers were washed with brine, dried over anhydrous magnesium sulfate, filtered and evaporated under reduced pressure. The residue was purified by silica gel column chromatography using dichloromethane/methanol 100/0 to 97/3 as eluent to afford 14-(2-nitrobenzenesulfonyl)-7,10-dioxa-4,14,19,20-tetraazatetracyclo[13.5.2.1^{2,6}.0^{18,21}]tricosa-1(20),2(23),3,5,15(22),16,18(21)-heptaene as a yellow oil.

LCMS method B: [M+H]⁺=496.1, t_{R}=0.80 min.

### Preparation of Example 4: 7,10-Dioxa-4,14,19,20-Tetraazatetracyclo[13.5.2.1^{2,6}.0^{18,21}] tricosa-1(20),2(23),3,5,15(22),16,18(21)-heptaene

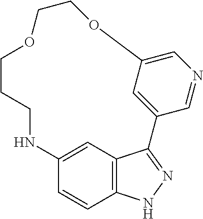 [SMILES: c1cc2[nH]nc3c2cc1NCCCOCCOc1cncc-3c1]

To a suspension of 14-(2-nitrobenzenesulfonyl)-7,10-dioxa-4,14,19,20-tetraazatetracyclo [13.5.2.1^{2,6}.0^{18,21}]tricosa-1(20),2(23),3,5,15(22),16,18(21)-heptaene (17 mg, 0.034 mmol) and cesium carbonate (10 mg, 0.102 mmol) in DMF (0.5 mL) at 0° C. was added thiophenol (10 μL, 0.102 mmol). The reaction mixture was stirred at RT for 2 h. Ethyl acetate and water were added and the aqueous layer was extracted with ethyl acetate. The combined organic layers were dried over anhydrous magnesium sulfate, filtered and evaporated under reduced pressure. The residue was purified by silica gel column chromatography using dichloromethne/methanol 100/0 to 97/3 as eluent to afford 7,10-dioxa-4,14,19,20-tetraazatetracyclo[13.5.2.1^{2,6}.0^{18,21}] tricosa-1(20),2(23),3,5,15(22),16,18(21)-heptaene as a solid.

LCMS method E: [M+H]⁺=311.2, t_{R}=1.901 min.

LCMS method D: [M+H]⁺=311.1, t_{R}=4.06 min.

¹H NMR (300 MHz, d6-DMSO) δ 12.94 (s, 1H), 8.75 (s, 1H), 8.41 (s, 1H), 8.15 (s, 1H), 7.38 (s, 1H), 7.34 (d, J=9.3 Hz, 1H), 6.83 (d, J=9.0 Hz, 1H), 5.93 (dd, J=18.7, 13.4 Hz, 1H), 4.49 (t, J=4.8 Hz, 2H), 3.80 (t, J=4.6 Hz, 2H), 3.54 (dt, J=21.4, 16.4 Hz, 3H), 1.98-1.70 (m, 2H) ppm.

### Example 5: 9,13-dioxa-4,5,18,19-tetraazatetracyclo[12.5.2.1^{2,6}.0^{17,20}]docosa-1(19),2(22), 3,14(21),15,17(20)-hexaene

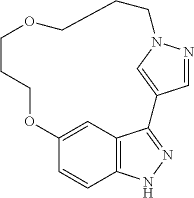 [SMILES: c1cc2[nH]nc3c2cc1OCCCOCCCn1cc-3cn1]

Example 5 is prepared according to the synthesis route described in general Scheme B.

### Preparation of Intermediate 43: 3-[3-(benzyloxy)propoxy]propan-1-ol

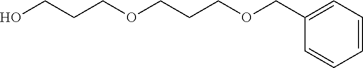 [SMILES: OCCCOCCCOCc1ccccc1]

To a solution of 1,3-propanediol (11.415 g, 150 mmol) in anhydrous DMF (85 mL) at 0° C. was added portionwise NaH (60% dispersion in mineral oil) (3.0 g, 75 mmol). After 1 h at 0° C., [(3-bromopropoxy)methyl]benzene (2.65 mL, 15 mmol) in DMF (5 mL) was added dropwise. The resulting reaction mixture was stirred at RT overnight. The reaction mixture was quenched by addition of water and the solvent was evaporated under reduced pressure. The residue was diluted with brine and extracted with ethyl acetate. The combined organic layers were dried over anhydrous sodium sulfate, filtered and evaporated under reduced pressure. The residue was purified by silica gel column chromatography using cyclohexane/ethyl acetate 100/00 to 30/70 as eluent to afford 3-[3-(benzyloxy)propoxy]propan-1-ol as a colorless oil.

LCMS method F: [M+H]⁺=225.2, t_{R}=2.1 min

### Preparation of Intermediate 44: 3-[3-(benzyloxy)propoxy]propyl methanesulfonate

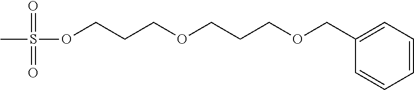 [SMILES: CS(=O)(=O)OCCCOCCCOCc1ccccc1]

To a solution of 3-[3-(benzyloxy)propoxy]propan-1-ol (2.5 g, 11.15 mmol) and triethylamine (3.107 mL, 22.29 mmol) in dichloromethane (45 mL) at 0° C. was added methanesulfonyl chloride (1.122 mL, 14.49 mmol) in dichloromethane (5 mL). The reaction mixture was stirred at RT overnight. The reaction mixture was diluted with brine and extracted with dichloromethane. The combined organic layers were dried over anhydrous sodium sulfate, filtered and evaporated under reduced pressure to afford 3-[3-(benzyloxy)propoxy]propyl methanesulfonate as a colorless oil which was used in the next step without further purification.

LCMS method F: [M+H]⁺=303.2, t_{R}=2.49 min

### Preparation of Intermediate 45: 1-{3-[3-(benzyloxy)propoxy]propyl}-4-(4,4,5,5-tetramethyl-1,3,2-dioxaborolan-2-yl)-1H-pyrazole

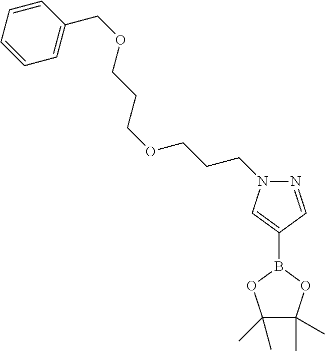 [SMILES: CC1(C)OB(c2cnn(CCCOCCCOCc3ccccc3)c2)OC1(C)C]

To a solution of 4-(4,4,5,5-tetramethyl-1,3,2-dioxaborolan-2-yl)-1H-pyrazole (2.225 g, 11.47 mmol) and 3-[3-(benzyloxy)propoxy]propylmethanesulfonate (2.89 g, 9.56 mmol) in anhydrous acetonitrile (80 mL) at RT was added cesium carbonate (4.048 g, 12.42 mmol). The reaction mixture was stirred at 70° C. overnight. The reaction mixture was filtered and the filtrate was concentrated under reduced pressure. The residue was purified by silica gel column chromatography using dichloromethane/ethyl acetate 100/0 to 80/20 as eluent to afford 1-{3-[3-(benzyloxy)propoxy]propyl}-4-(4,4,5,5-tetramethyl-1,3,2-dioxaborolan-2-yl)-1H-pyrazole as a colorless oil.

LCMS method F: [M+H]⁺=401.3, t_{R}=2.95 min

### Preparation of Intermediate 46: 3-(1-{3-[3-(benzyloxy)propoxy]propyl}-1H-pyrazol-4-yl)-5-[(tert-butyldimethylsilyl)oxy]-1-(oxan-2-yl)-1H-indazole

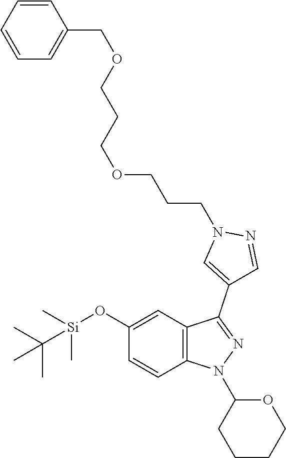 [SMILES: CC(C)(C)[Si](C)(C)Oc1ccc2c(c1)c(-c1cnn(CCCOCCCOCc3ccccc3)c1)nn2C1CCCCO1]

To a solution of 5-[(tert-butyldimethylsilyl)oxy]-3-iodo-1-(oxan-2-yl)-1H-indazole (2.337 g, 5.1 mmol) in dioxane (22.95 mL) and water (2.55 mL) at RT was added 1-{3-[3-(benzyloxy)propoxy]propyl}-4-(4,4,5,5-tetramethyl-1,3,2-dioxaborolan-2-yl)-1H-pyrazole (2.143 g, 5.35 mmol), potassium phosphate tribasic (3.247 g, 15.29 mmol), XPhos (243 mg, 0.51 mmol) and Pd(PPh₃)₄ (295 mg, 0.25 mmol). The resulting reaction mixture was stirred at 90° C. for 24 h. The residue was diluted with brine and extracted with ethyl acetate. The combined organic layers were dried over anhydrous sodium sulfate and concentrated under reduced pressure. The residue was purified by silica gel column chromatography cyclohexane/ethyl acetate 100/0 to 70/30 as eluent to afford 3-(1-{3-[3-(benzyloxy)propoxy] propyl}-1H-pyrazol-4-yl)-5-[(tert-butyldimethylsilyl)oxy]-1-(oxan-2-yl)-1H-indazole as a colorless oil.

LCMS method F: [M+H]⁺=605.5, t_{R}=3.81 min

### Preparation of Intermediate 47: 3-[3-(4-{5-[(tert-butyldimethylsilyl)oxy]-1-(oxan-2-yl)-1H-indazol-3-yl}-1H-pyrazol-1-yl)propoxy]propan-1-ol

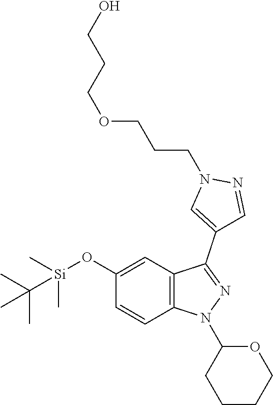 [SMILES: CC(C)(C)[Si](C)(C)Oc1ccc2c(c1)c(-c1cnn(CCCOCCCO)c1)nn2C1CCCCO1]

To a solution of 3-(1-{3-[3-(benzyloxy)propoxy]propyl}-1H-pyrazol-4-yl)-5-[(tert-butyl dimethylsilyl)oxy]-1-(oxan-2-yl)-1H-indazole (1.58 g, 2.61 mmol) in ethanol (30 mL) at RT was added palladium 10% on carbon (158 mg). The reaction mixture was stirred under hydrogen atmosphere at RT overnight. The reaction mixture was filtered and the filtrate was concentrated under reduced pressure. The residue was purified by silica gel column chromatography using cyclohexane/ethyl acetate 100/0 to 20/80 as eluent to afford 3-[3-(4-{5-[(tert-butyldimethylsilyl)oxy]-1-(oxan-2-yl)-1H-indazol-3-yl}-1H-pyrazol-1-yl)propoxy] propan-1-ol as a colorless oil.

LCMS method F: [M+H]⁺=515.4, t_{R}=3.27 min

### Preparation of Intermediate 48: 3-[3-(4-{5-[(tert-butyldimethylsilyl)oxy]-1-(oxan-2-yl)-1H-indazol-3-yl}-1H-pyrazol-1-yl)propoxy]propyl methanesulfonate

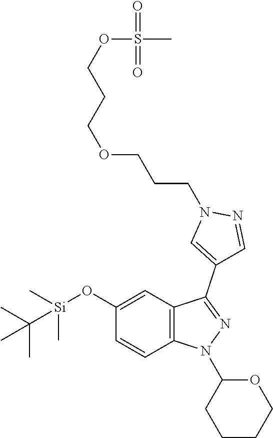 [SMILES: CC(C)(C)[Si](C)(C)Oc1ccc2c(c1)c(-c1cnn(CCCOCCCOS(C)(=O)=O)c1)nn2C1CCCCO1]

To a solution of 3-[3-(4-{5-[(tert-butyldimethylsilyl)oxy]-1-(oxan-2-yl)-1H-indazol-3-yl}-1H-pyrazol-1-yl)propoxy]propan-1-ol (700 mg, 1.36 mmol) and triethylamine (379 μL, 2.72 mmol) in dichloromethane (10 mL) at 0° C. was added methanesulfonyl chloride (137 μL, 1.77 mmol) in dichloromethane (2 mL). The reaction mixture was stirred at RT overnight. The residue was diluted with brine and extracted with dichloromethane. The combined organic layers were dried over anhydrous sodium sulfate, filtered and evaporated under reduced pressure to afford 3-[3-(4-{5-[(tert-butyldimethylsilyl)oxy]-1-(oxan-2-yl)-1H-indazol-3-yl}-1H-pyrazol-1-yl) propoxy]propylmethanesulfonate as a colorless oil which was used in the next step without further purification.

LCMS method F: [M+H]⁺=593.3, t_{R}=3.42 min

### Preparation of Intermediate 49: 3-(3-{4-[5-hydroxy-1-(oxan-2-yl)-1H-indazol-3-yl]-1H-pyrazol-1-yl}propoxy)propyl methanesulfonate

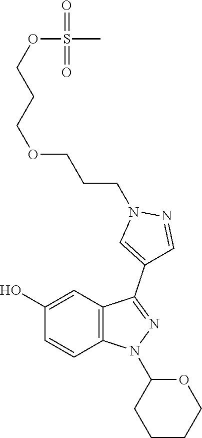 [SMILES: CS(=O)(=O)OCCCOCCCn1cc(-c2nn(C3CCCCO3)c3ccc(O)cc23)cn1]

To a solution of 3-[3-(4-{5-[(tert-butyldimethylsilyl)oxy]-1-(oxan-2-yl)-1H-indazol-3-yl}-1H-pyrazol-1-yl)propoxy]propyl methanesulfonate (806 mg, 1.36 mmol) in THF (5 mL) at RT was added tetrabutylammonium fluoride (1M solution in THF) (2.04 mL, 2.04 mmol). The reaction mixture was stirred at RT for 2 h. The reaction mixture was diluted brine and extracted with ethyl acetate. The combined organic layers were dried over anhydrous sodium sulfate, filtered and evaporated under reduced pressure. The residue was purified by silica gel column chromatography using dichloromethane/ethyl acetate 100/0 to 0/100 as eluent to afford 3-(3-{4-[5-hydroxy-1-(oxan-2-yl)-1H-indazol-3-yl]-1H-pyrazol-1-yl}propoxy)propylmethane sulfonate as a colorless oil.

LCMS method F: [M+H]⁺=479.2, t_{R}=2.29 min

### Preparation of Intermediate 50: 18-(oxan-2-yl)-9,13-dioxa-4,5,18,19-tetraazatetra cyclo[12.5.2.1^{2,5}.0^{17,20}]docosa-1(19),2(22),3,14(21),15,17(20)-hexaene

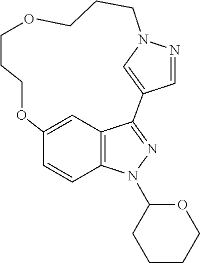 [SMILES: c1cc2c3cc1OCCCOCCCn1cc(cn1)-c3nn2C1CCCCO1]

A suspension of cesium carbonate (368 mg, 1.13 mmol) in anhydrous DMF (100 mL) at 80° C. was added dropwise to a solution of 3-(3-{4-[5-hydroxy-1-(oxan-2-yl)-1H-indazol-3-yl]-1H-pyrazol-1-yl}propoxy)propyl methanesulfonate (180 mg, 0.38 mmol) in DMF (90 mL). The reaction mixture was stirred at 80° C. for 30 min. The solvent was evaporated under reduced pressure, diluted with brine and extracted with ethyl acetate. The combined organic layers were dried over anhydrous sodium sulfate, filtered and evaporated under reduced pressure. The residue was purified by silica gel column chromatography using dichloromethane/ethyl acetate 100/0 to 60/40 as eluent to afford 18-(oxan-2-yl)-9,13-dioxa-4,5,18,19-tetraazatetracyclo [12.5.2.1^{2,5}.0^{17,20}]docosa-1(19),2(22),3,14(21),15,17(20)-hexaene as a colorless oil.

LCMS method F: [M+H]⁺=383.3, t_{R}=2.60 min

### Preparation of Example 5: 9,13-dioxa-4,5,18,19-tetraazatetracyclo[12.5.2.1^{2,5}.0^{17,20}] docosa-1(19),2(22),3,14(21),15,17(20)-hexaene

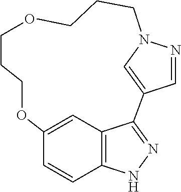 [SMILES: c1cc2[nH]nc3c2cc1OCCCOCCCn1cc-3cn1]

To a solution of 18-(oxan-2-yl)-9,13-dioxa-4,5,18,19-tetraazatetracyclo[12.5.2.1^{2,5}.0^{17,20}] docosa-1(19),2(22),3,14(21),15,17(20)-hexaene (128 mg, 0.33 mmol) in dichloromethane (3 mL) at RT was added TFA (498 μL, 6.69 mmol). The reaction mixture was stirred under microwave irradiations at 80° C. for 1 h. The solvent was evaporated under reduced pressure and the residue was diluted with brine and extracted with ethyl acetate. The combined organic layers were dried over anhydrous sodium sulfate, filtered and evaporated under reduced pressure. The residue was purified by silica gel column chromatography using cyclohexane/(ethyl acetate/ethanol (3/1)) 100/0 to 60/40 as eluent. The resulting solid was triturated with acetonitrile, filtered and dried to afford 9,13-dioxa-4,5,18,19-tetraazatetracyclo [12.5.2.1^{2,5}.0^{17,20}]docosa-1(19),2(22),3,14(21),15,17(20)-hexaene as a solid.

LCMS method F: [M+H]⁺=299.2, t_{R}=2.07 min

LCMS method G: [M+H]⁺=299.3, t_{R}=2.09 min

¹H NMR (400 MHz, d6-DMSO) δ 12.69 (1H, s), 8.62 (1H, d, J=0.6 Hz), 7.67-7.67 (1H, m), 7.47-7.38 (2H, m), 6.94 (1H, dd, J=2.3, 8.9 Hz), 4.40-4.28 (4H, m), 3.57-3.52 (2H, m), 3.33-3.33 (2H, m), 2.17-2.04 (4H, m) ppm.

### Example 6: 10,14-dioxa-4,5,19,20-tetraazatetracyclo[13.5.2.1^{2,5}.0^{18,21}]tricosa-1(20),2(23), 3,15(22),16,18(21)-hexaene

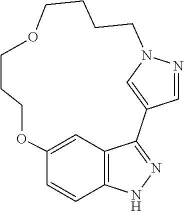 [SMILES: c1cc2[nH]nc3c2cc1OCCCOCCCCn1cc-3cn1]

Example 6 is prepared according to the synthesis route described in general Scheme B.

### Preparation of Intermediate 51: 4-[3-(benzyloxy)propoxy]butan-1-ol

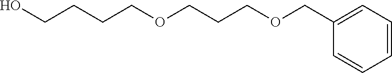 [SMILES: OCCCCOCCCOCc1ccccc1]

To a solution of 1,4-butanediol (9.01 g, 100 mmol) in anhydrous DMF (55 mL) at 0° C. was added portionwise NaH (60% dispersion in mineral oil) (2 g, 50 mmol). After 1 h at 0° C., [(3-bromopropoxy)methyl]benzene (1.77 mL, 10 mmol) in DMF (5 mL) was added dropwise. The reaction mixture was stirred at RT overnight. The reaction mixture was quenched by addition of water and the solvent was evaporated under reduced pressure. The residue was diluted with brine and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 30/70 as eluent to afford 4-[3-(benzyloxy)propoxy]butan-1-ol as a colorless oil.

LCMS method F: [M+H]⁺=239.2, t_{R}=2.22 min

### Preparation of Intermediate 52: 4-[3-(benzyloxy)propoxy]butyl methanesulfonate

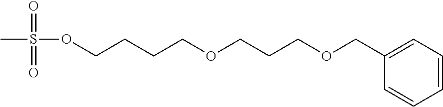 [SMILES: CS(=O)(=O)OCCCCOCCCOCc1ccccc1]

To a solution of 4-[3-(benzyloxy)propoxy]butan-1-ol (2.26 g, 9.48 mmol) and triethylamine (2.644 mL, 18.97 mmol) in dichloromethane (35 mL) at 0° C. was added dropwise methanesulfonyl chloride (955 μL, 12.33 mmol) in dichloromethane (5 mL). The reaction mixture was stirred at RT overnight. The reaction mixture was diluted with brine and extracted with dichloromethane. The combined organic layers were dried over anhydrous sodium sulfate, filtered and evaporated under reduced pressure to afford 4-[3-(benzyloxy)propoxy]butyl methanesulfonate as a colourless oil which was used in the next step without further purification.

LCMS method F: [M+H]⁺=317.2, t_{R}=2.59 min

### Preparation of Intermediate 53: 1-{4-[3-(benzyloxy)propoxy]butyl}-4-(4,4,5,5-tetramethyl-1,3,2-dioxaborolan-2-yl)-1H-pyrazole

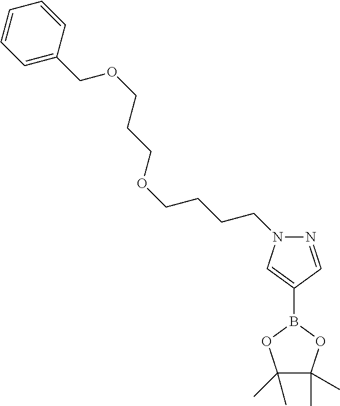 [SMILES: CC1(C)OB(c2cnn(CCCCOCCCOCc3ccccc3)c2)OC1(C)C]

To a solution of 4-(4,4,5,5-tetramethyl-1,3,2-dioxaborolan-2-yl)-1H-pyrazole (3 g, 9.48 mmol) in acetonitrile (40 mL) at RT was added cesium carbonate (4.634 g, 14.22 mmol) and 4-[3-(benzyloxy)propoxy]butyl methanesulfonate (2.023 g, 10.43 mmol). The resulting reaction mixture was stirred at 70° C. overnight. The reaction mixture was filtered and the filtrate was concentrated under reduced pressure. The residue was purified by silica gel column chromatography using cyclohexane/(ethyl acetate/ethanol (3/1)) 100/0 to 90/10 as eluent to afford 1-{4-[3-(benzyloxy)propoxy]butyl}-4-(4,4,5,5-tetramethyl-1,3,2-dioxaborolan-2-yl)-1H-pyrazole as a colorless oil.

LCMS method F: [M+H]⁺=415.3, t_{R}=3.02 min

### Preparation of Intermediate 54: 3-(1-{4-[3-(benzyloxy)propoxy]butyl}-1H-pyrazol-4-yl)-1-(oxan-2-yl)-1H-indazol-5-ol

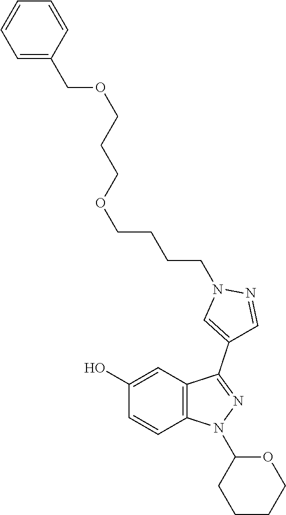 [SMILES: Oc1ccc2c(c1)c(-c1cnn(CCCCOCCCOCc3ccccc3)c1)nn2C1CCCCO1]

To a solution of 5-[(tert-butyldimethylsilyl)oxy]-3-iodo-1-(oxan-2-yl)-1H-indazole (1.834 g, 4 mmol) in dioxane (36 mL) and water (4 mL) at RT was added 1-{4-[3-(benzyloxy)propoxy]butyl}-4-(4,4,5,5-tetramethyl-1,3,2-dioxaborolan-2-yl)-1H-pyrazole (2.155 g, 5.2 mmol), potassium phosphate tribasic (2.548 g, 12 mmol), XPhos (191 mg, 0.4 mmol) and Pd(PPh₃)₄ (231 mg, 0.2 mmol). The reaction mixture was stirred at 90° C. for 24 h. The residue was diluted with brine and extracted with ethyl acetate. The combined organic layers were dried over anhydrous sodium sulfate, filtered and the solvent was evaporated under reduced pressure. The residue was purified by silica gel column chromatography dichloromethane/ethyl acetate 100/0 to 50/50 as eluent to afford 3-(1-{4-[3-(benzyloxy)propoxy]butyl}-1H-pyrazol-4-yl)-1-(oxan-2-yl)-1H-indazol-5-ol as a colorless oil.

LCMS method F: [M+H]⁺=505.5, t_{R}=2.84 min

### Preparation of Intermediate 55: 3-(1-{4-[3-(benzyloxy)propoxy]butyl}-1H-pyrazol-4-yl)-5-[(tert-butyldimethylsilyl)oxy]-1-(oxan-2-yl)-1H-indazole

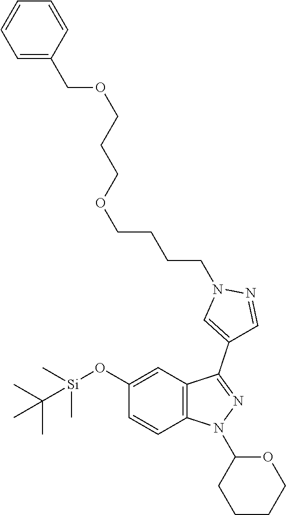 [SMILES: CC(C)(C)[Si](C)(C)Oc1ccc2c(c1)c(-c1cnn(CCCCOCCCOCc3ccccc3)c1)nn2C1CCCCO1]

To a solution of 3-(1-{4-[3-(benzyloxy)propoxy]butyl}-1H-pyrazol-4-yl)-1-(oxan-2-yl)-1H-indazol-5-ol (1.8 g, 3.57 mmol) in DMF (10 mL) at 0° C. was added imidazole (292 mg, 4.28 mmol) and TBDMSCl (591 mg, 3.92 mmol). The reaction mixture was stirred at RT overnight. The reaction was poured into water and extracted ethyl acetate. The organic layer was dried over anhydrous sodium sulfate, filtered and evaporated under reduced pressure. The residue was purified by silica gel column chromatography using cyclohexane/ethyl acetate 100/0 to 70/30 as eluent to afford 3-(1-{4-[3-(benzyloxy)propoxy]butyl}-1H-pyrazol-4-yl)-5-[(tert-butyldimethylsilyl)oxy]-1-(oxan-2-yl)-1H-indazole as a colorless oil.

LCMS method F: [M+H]⁺=619.5, t_{R}=3.84 min

### Preparation of Intermediate 56: 3-[4-(4-{5-[(tert-butyldimethylsilyl)oxy-]-(oxan-2-yl)-1H-indazol-3-yl}-1H-pyrazol-1-yl)butoxy]propan-1-ol

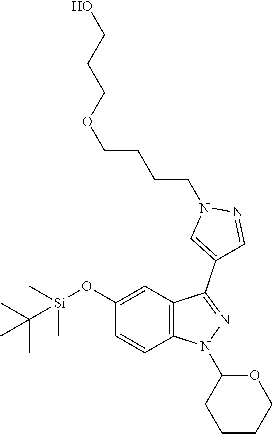 [SMILES: CC(C)(C)[Si](C)(C)Oc1ccc2c(c1)c(-c1cnn(CCCCOCCCO)c1)nn2C1CCCCO1]

To a solution of 3-(1-{4-[3-(benzyloxy)propoxy]butyl}-1H-pyrazol-4-yl)-5-[(tert-butyl dimethylsilyl)oxy]-1-(oxan-2-yl)-1H-indazole (1.25 g, 2.02 mmol) in ethanol (30 mL) at RT was added palladium 10% on carbon (125 mg) The reaction mixture was stirred under hydrogen atmosphere at RT overnight. The reaction mixture was filtered and the filtrate was concentrated under reduced pressure. The residue was purified by silica gel column chromatography using cyclohexane/ethyl acetate 100/0 to 20/80 as eluent to afford 3-[4-(4-{5-[(tert-butyldimethylsilyl)oxy]-1-(oxan-2-yl)-1H-indazol-3-yl}-1H-pyrazol-1-yl)butoxy] propan-1-ol as a colorless oil.

LCMS method F: [M+H]⁺=529.4, t_{R}=3.31 min

### Preparation of Intermediate 57: 3-[4-(4-{5-[(tert-butyldimethylsilyl)oxy]-1-(oxan-2-yl)-1H-indazol-3-yl}-1H-pyrazol-1-yl)butoxy]propyl methanesulfonate

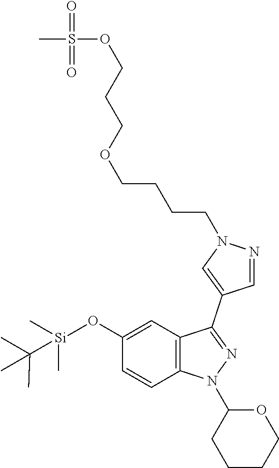 [SMILES: CC(C)(C)[Si](C)(C)Oc1ccc2c(c1)c(-c1cnn(CCCCOCCCOS(C)(=O)=O)c1)nn2C1CCCCO1]

To a solution of 3-[4-(4-{5-[(tert-butyldimethylsilyl)oxy]-1-(oxan-2-yl)-1H-indazol-3-yl}-1H-pyrazol-1-yl) butoxy]propan-1-ol (650 mg, 1.23 mmol) and triethylamine (343 □L, 2.46 mmol) in dichloromethane (10 mL) at 0° C. was added methanesulfonyl chloride (124 □L, 1.60 mmol) in dichloromethane (2 mL). The reaction mixture was stirred at RT for 2 h. The residue was diluted with brine and extracted with dichloromethane. The combined organic layers were dried over anhydrous sodium sulfate, filtered and evaporated under reduced pressure to afford 3-[4-(4-{5-[(tert-butyldimethylsilyl)oxy]-1-(oxan-2-yl)-1H-indazol-3-yl}-1H-pyrazol-1-yl)butoxy] propylmethanesulfonate as a colorless oil which was used in the next step without further purification.

LCMS method F: [M+H]⁺=607.4, t_{R}=3.45 min

### Preparation of Intermediate 58: 3-(4-{4-[5-hydroxy-1-(oxan-2-yl)-1H-indazol-3-yl]-1H-pyrazol-1-yl}butoxy)propyl methanesulfonate

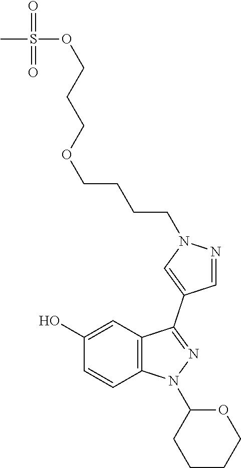 [SMILES: CS(=O)(=O)OCCCOCCCCn1cc(-c2nn(C3CCCCO3)c3ccc(O)cc23)cn1]

To a solution of 3-[4-(4-{5-[(tert-butyldimethylsilyl)oxy]-1-(oxan-2-yl)-1H-indazol-3-yl}-1H-pyrazol-1-yl)butoxy]propyl methanesulfonate (746 mg, 1.23 mmol) in THF (5 mL) at 0° C. was added TBAF (1M solution in THF) (1.35 mL, 1.35 mmol). The reaction mixture was stirred at RT for 2 h. The reaction mixture was diluted with saturated ammonium chloride solution and extracted with ethyl acetate. The combined organic layers were dried over anhydrous sodium sulfate, filtered and evaporated under reduced pressure. The residue was purified by silica gel column chromatography using dichloromethane/ethyl acetate 100/0 to 40/60 as eluent to afford 3-(4-{4-[5-hydroxy-1-(oxan-2-yl)-1H-indazol-3-yl]-1H-pyrazol-1-yl}butoxy)propylmethane sulfonate as a colorless oil.

LCMS method F: [M+H]⁺=493.3, t_{R}=2.24 min

### Preparation of Intermediate 59: 19-(oxan-2-yl)-10,14-dioxa-4,5,19,20-tetraazatetra cyclo [13.5.2.1^{2,5}.0^{18,21}]tricosa-1(20),2(23),3,15(22),16,18(21)-hexaene

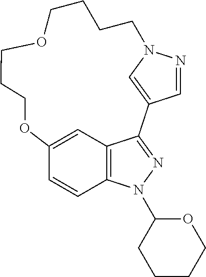 [SMILES: c1cc2c3cc1OCCCOCCCCn1cc(cn1)-c3nn2C1CCCCO1]

To a suspension of cesium carbonate (893 mg, 2.74 mmol) in DMF (92 mL) at 80° C. was added dropwise a solution of 3-(4-{4-[5-hydroxy-1-(oxan-2-yl)-1H-indazol-3-yl]-1H-pyrazol-1-yl}butoxy)propyl methanesulfonate (450 mg, 0.91 mmol) in DMF (90 mL). The reaction mixture was stirred at 80° C. for 30 min. The solvent was evaporated under reduced pressure and the residue was diluted with brine and extracted with ethyl acetate. The combined organic layers were dried over anhydrous sodium sulfate, filtered and evaporated under reduced pressure. The residue was purified by silica gel column chromatography using dichloromethane/ethyl acetate 100/0 to 60/40 as eluent to afford 19-(oxan-2-yl)-10,14-dioxa-4,5,19,20-tetraazatetracyclo [13.5.2.1^{2,5}.0^{18,21}]tricosa-1(20),2(23),3,15(22),16,18(21)-hexaene as a cream solid.

LCMS method F: [M+H]⁺=397.2, t_{R}=2.82 min

### Preparation of Example 6: 10,14-dioxa-4,5,19,20-tetraazatetracyclo[13.5.2.1^{2,5}.0^{18,21}] tricosa-1(20),2(23),3,15(22),16,18(21)-hexaene

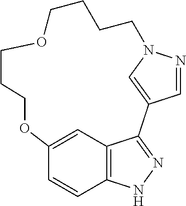 [SMILES: c1cc2[nH]nc3c2cc1OCCCOCCCCn1cc-3cn1]

To a solution of 19-(oxan-2-yl)-10,14-dioxa-4,5,19,20-tetraazatetracyclo[13.5.2.1^{2,5}.0^{18,21}] tricosa-1(20),2(23),3,15(22),16,18(21)-hexaene (290 mg, 0.73 mmol) in dichloromethane (3 mL) at RT was added TFA (1.089 mL, 14.63 mmol). The reaction mixture was stirred under microwave conditions at 80° C. for 1 h. The solvent was evaporated under reduced pressure, diluted with saturated sodium bicarbonate solution and extracted with ethyl acetate. The organic layer was dried over anhydrous sodium sulfate, filtered and evaporated under reduced pressure. The residue was purified by silica gel column chromatography using cyclohexane/(ethyl acetate/ethanol (3/1)) 100/0 to 50/50 as eluent. The resulting solid was triturated with acetonitrile, filtered and dried to afford 10,14-dioxa-4,5,19,20-tetraazatetracyclo [13.5.2.1^{2,5}.0^{18,21}]tricosa-1(20),2(23),3,15(22),16,18(21)-hexaene as a solid.

LCMS method F: [M+H]⁺=313.3, t_{R}=2.14 min

LCMS method G: [M+H]⁺=313.3, t_{R}=2.15 min H NMR (400 MHz, d6-DMSO) δ 12.75 (1H, s), 8.34 (1H, s), 7.78 (1H, s), 7.43-7.36 (2H, m), 6.95 (1H, dd, J=2.4, 9.0 Hz), 4.30-4.19 (4H, m), 3.34-3.33 (4H, m), 2.14-2.02 (4H, m), 1.62-1.56 (2H, m) ppm.

### Example 7: (13R)-13-methyl-7,11,14-trioxa-5,19,20,23-tetraazatetracyclo[13.5.2.1^{2,6}.0^{18,21}]tricosa-1(20),2(23),3,5,15(22),16,18(21)-heptaene

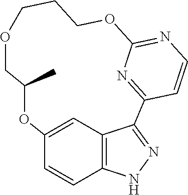 [SMILES: C[C@@H]1COCCCOc2nccc(n2)-c2n[nH]c3ccc(cc23)O1]

Example 7 is prepared according to the synthesis route described in general Scheme C.

### Preparation of Intermediate 60: tert-butyl-dimethyl-(1-tetrahydropyran-2-ylindazol-5-yl)oxy-silane

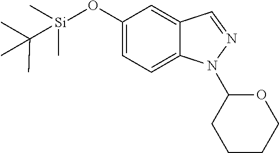 [SMILES: CC(C)(C)[Si](C)(C)Oc1ccc2c(cnn2C2CCCCO2)c1]

To a solution of tert-butyl-(1H-indazol-5-yloxy)-dimethyl-silane (47.9 g, 193.04 mmol) in chloroform (1.5 L) at RT was added p-toluenesulfonic acid monohydrate (3.67 g, 19.3 mmol) and DHP (52.83 mL, 579.13 mmol). The reaction mixture was stirred at 50° C. 2 h. The solvent was removed under reduced pressure and the residue was dissolved in ethyl acetate and washed with water. The organic layer was dried over anhydrous sodium sulfate, filtered and evaporated under reduced pressure. The residue was purified by silica gel column chromatography using cyclohexane/ethyl acetate 100/0 to 95/5 as eluent to afford tert-butyl-dimethyl-(1-tetrahydropyran-2-ylindazol-5-yl)oxy-silane as a orange oil.

LCMS method F: [M+H]⁺=333.3, t_{R}=3.51 min

### Preparation of Intermediate 61: tert-butyl-dimethyl-[1-tetrahydropyran-2-yl-3-(4,4,5,5-tetramethyl-1,3,2-dioxaborolan-2-yl)indazol-5-yl]oxy-silane

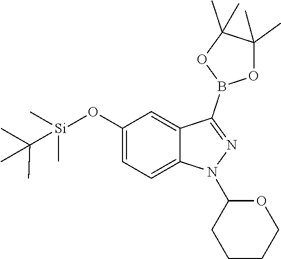 [SMILES: CC1(C)OB(c2nn(C3CCCCO3)c3ccc(O[Si](C)(C)C(C)(C)C)cc23)OC1(C)C]

To a solution of tert-butyl-dimethyl-(1-tetrahydropyran-2-ylindazol-5-yl)oxy-silane (10.5 g; 31.61 mmol) in TBME (60 mL) in a Schlenk tube was added 4,4,5,5-tetramethyl-2-(4,4,5,5-tetramethyl-1,3,2-dioxaborolan-2-yl)-1,3,2-dioxaborolane (8.06 g; 31.61 mmol), 4,4′-di-tert-butyl-2,2′-bipyridine (509 mg, 1.90 mmol) and (1,5-cyclooctadiene)(methoxy)iridium(I) dimer (439 mg; 0.66 mmol). The reaction mixture was purged with argon for 10 min then stirred at 80° C. overnight. The solvent was removed under reduced pressure and the residue was dissolved with ethyl acetate and water. The layers were separated and the aqueous layer was extracted with ethyl acetate. The combined organic layers were washed with 1N aqueous sodium hydride solution and water. The organic layer was dried over anhydrous sodium sulfate, filtered and evaporated under reduced pressure to afford tert-butyl-dimethyl-[1-tetrahydropyran-2-yl-3-(4,4,5,5-tetramethyl-1,3,2-dioxaborolan-2-yl)indazol-5-yl]oxy-silane as a light yellow solid.

LCMS method F: [M+H]⁺=459.4, t_{R}=3.86 min

### Preparation of Intermediate 62: tert-butyl-dimethyl-[3-(2-methylsulfanylpyrimidin-4-yl)-1-tetrahydropyran-2-yl-indazol-5-yl]oxy-silane

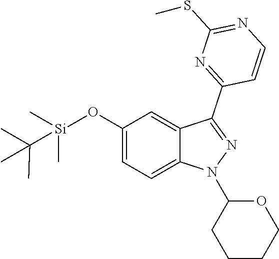 [SMILES: CSc1nccc(-c2nn(C3CCCCO3)c3ccc(O[Si](C)(C)C(C)(C)C)cc23)n1]

Tert-butyl-dimethyl-[1-tetrahydropyran-2-yl-3-(4,4,5,5-tetramethyl-1,3,2-dioxaborolan-2-yl) indazol-5-yl]oxy-silane (2.8 g, 6.107 mmol), 4-chloro-2-methylsulfanyl-pyrimidine (701 mg, 4.362 mmol), XPhos (208 mg, 0.436 mmol) and potassium phosphate tribasic (2.778 g, 13.086 mmol) were suspended in dioxane (42.5 mL) and water (2.5 mL) In a Schlenk tube and the mixture was degassed with nitrogen for 15 min. Pd(PPh₃)₄ (252 mg, 0.218 mmol) was added and the sealed vial was heated at 90° C. for 2 h. The reaction mixture was cooled to RT, poured in ethyl acetate and water. The layers were separated. The aqueous layer was extracted with ethyl acetate and the organic layer was washed with brine, dried over anhydrous sodium sulfate, filtered and evaporated under reduced pressure. The residue was purified by silica gel column chromatography using cyclohexane/ethyl acetate 100/0 to 70/30 as eluent to afford tert-butyl-dimethyl-[3-(2-methylsulfanylpyrimidin-4-yl)-1-tetrahydropyran-2-yl-indazol-5-yl]oxy-silane as an orange gum.

LCMS method I: [M+H]⁺=457.4, t_{R}=3.63 min

### Preparation of Intermediate 63: tert-butyl-dimethyl-[3-(2-methylsulfonylpyrimidin-4-yl)-1-tetrahydropyran-2-yl-indazol-5-yl]oxy-silane

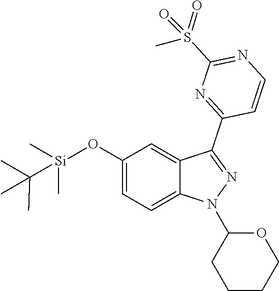 [SMILES: CC(C)(C)[Si](C)(C)Oc1ccc2c(c1)c(-c1ccnc(S(C)(=O)=O)n1)nn2C1CCCCO1]

To a solution of tert-butyl-dimethyl-[3-(2-methylsulfanylpyrimidin-4-yl)-1-tetrahydropyran-2-yl-indazol-5-yl]oxy-silane (1.866 g, 4.086 mmol) and ammonium molybdate (168 mg, 0.136 mmol) in ethanol (40 mL) was added hydrogen peroxyde (30% wt aqueous solution) (1.13 mL, 36.774 mmol) dropwise over 5 min. The reaction mixture was stirred at 0° C. for 5 min and was stirred at RT for 62 h. Additional H₂O₂ (30% wt aqueous solution) (1.13 mL, 36.774 mmol) was added and the reaction mixture was heated at 50° C. The solvent was evaporated under reduced pressure and the residue was partitioned between ethyl acetate and water. The aqueous layer was extracted with ethyl acetate and the combined organic layers were washed with brine, dried over anhydrous sodium sulfate, filtered and evaporated under reduced pressure to afford tert-butyl-dimethyl-[3-(2-methylsulfonylpyrimidin-4-yl)-1-tetrahydropyran-2-yl-indazol-5-yl]oxy-silane as a yellow solid.

LCMS method I: [M+H]⁺=489.4, t_{R}=2.77 min

### Preparation of Intermediate 64: (S)-1-((tert-butyldiphenylsilyl)oxy)propan-2-ol

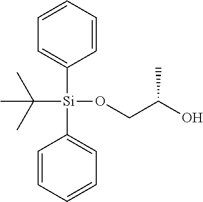 [SMILES: C[C@H](O)CO[Si](c1ccccc1)(c1ccccc1)C(C)(C)C]

To a solution of (S)-(+)-1,2-propanediol (15.165 g, 199.27 mmol) in THF (1000 mL) at RT were sequentially added imidazole (16.28 g, 239.132 mmol) and tert-butyldiphenylchlorosilane (53.75 mL, 209.24 mmol). The reaction mixture was stirred at RT for 15 h. The reaction mixture was diluted with ethyl acetate and washed with a saturated aqueous NaHCO₃ solution then brine. The organic layer was dried over anhydrous magnesium sulfate, filtered and evaporated under reduced pressure. The residue was purified by silica gel chromatography using heptane/ethyl acetate 100/0 to 50/50/as eluent to afford (S)-1-((tert-butyldiphenylsilyl) oxy)propan-2-ol as a colorless oil.

LCMS method B: [M+H]⁺=not detected, t_{R}=1.258 min

### Preparation of Intermediate 65: (S)-(2-(benzyloxy)propoxy)(tert-butyl)diphenylsilane

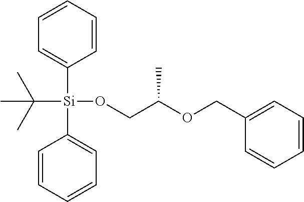 [SMILES: C[C@@H](CO[Si](c1ccccc1)(c1ccccc1)C(C)(C)C)OCc1ccccc1]

In a pressure flask were added (S)-1-((tert-butyldiphenylsilyl)oxy)propan-2-ol (30 g, 95.39 mmol), benzyl bromide (16.995 mL, 143.08 mmol), DIPEA (26.586 mL, 152.62 mmol) and potassium iodide (1.584 g, 9.53 mmol). The reaction mixture was stirred at 150° C. for 15 h. The reaction mixture was diluted with ethyl acetate and washed with a saturated aqueous NaHCO₃ solution. The aqueous phase was extracted with ethyl acetate. The organic layer was washed with brine, dried over anhydrous magnesium sulfate, filtered and evaporated under reduced pressure. The residue was purified by silica gel column chromatography using heptane/ethyl acetate 100/0 to 95/5 as eluent to afford (S)-(2-(benzyloxy)propoxy)(tert-butyl)diphenylsilane as a colorless oil.

LCMS method E: [M+H]⁺=not detected, t_{R}=5.496 min

### Preparation of Intermediate 66: (S)-2-(benzyloxy)propan-1-ol

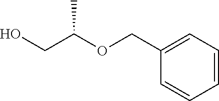

To a solution of (S)-(2-(benzyloxy)propoxy)(tert-butyl)diphenylsilane (23 g, 138.37 mmol) in THE (415 mL) at 0° C. was added TBAF (1M solution in THF) (208 mL, 208 mmol). The reaction mixture was stirred at RT for 15 h. The reaction mixture was diluted with ethyl acetate, washed with a saturated aqueous NaHCO₃ solution and brine. The organic layer was dried over anhydrous magnesium sulfate, filtered and evaporated under reduced pressure. The residue was purified by silica gel column chromatography using heptane/ethyl acetate 100/0 to 80/20 as eluent to afford (S)-2-(benzyloxy)propan-1-ol as a colorless oil.

LCMS method B: [M+H]⁺=167.0, t_{R}=0.508 min

### Preparation of Intermediate 67: 3-[(2S)-2-benzyloxypropoxy]propoxy-tert-butyl-dimethyl-silane

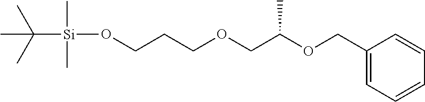 [SMILES: C[C@@H](COCCCO[Si](C)(C)C(C)(C)C)OCc1ccccc1]

To a solution of (S)-2-(benzyloxy)propan-1-ol (4 g, 24.064 mmol) in dry THF (121 mL) at 0° C. under nitrogen atmosphere was added sodium hydride (60% dispersion in mineral oil) (2.88 g, 72.192 mmol) The reaction mixture was stirred at 0° C. for 1 h. 3-bromopropoxy)-tert-butyldimethylsilane (6.71 mL, 28.877 mmol) was added and the reaction mixture was stirred at 80° C. for 15 h. The reaction mixture was cooled to 0° C., diluted with ethyl acetate and quenched with a saturated aqueous ammonium chloride solution. The organic layer was washed with brine, dried over anhydrous magnesium sulfate, filtered and evaporated under reduced pressure. The residue was purified by silica gel column chromatography using heptane/ethyl acetate 99/1 to 90/10 as eluent to afford 3-[(2S)-2-benzyloxypropoxy]propoxy-tert-butyl-dimethyl-silane as a colorless oil.

LCMS method B: [M+H]⁺=339.1, t_{R}=1.479 min

### Preparation of Intermediate 68: (S)-3-(2-(benzyloxy)propoxy)propan-1-ol

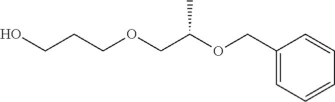 [SMILES: C[C@@H](COCCCO)OCc1ccccc1]

To a solution of 3-[(2S)-2-benzyloxypropoxy]propoxy-tert-butyl-dimethyl-silane (1.52 g, 4.489 mmol) in THF (23 mL) at 0° C. was added TBAF (1M solution in THF) (6.734 mL, 6.734 mmol). The reaction mixture was stirred at RT for 15 h. The reaction mixture was diluted with ethyl acetate, washed with a saturated aqueous NaHCO₃ solution, brine and water. The pH was adjusted to pH 4-5 using 1N HCl. The organic layer was dried over anhydrous magnesium sulfate, filtered and evaporated under reduced pressure. The residue was purified by silica gel column chromatography using heptane/ethyl acetate 100/0 to 90/10 as eluent to afford (S)-3-(2-(benzyloxy)propoxy)propan-1-ol as a colourless oil.

LCMS method B: [M+H]⁺=225.0, t_{R}=0.640 min

### Preparation of Intermediate 69: [3-[2-[3-[(2S)-2-benzyloxypropoxy]propoxy] pyrimidin-4-yl]-1-tetrahydropyran-2-yl-indazol-5-yl]oxy-tert-butyl-dimethyl-silane

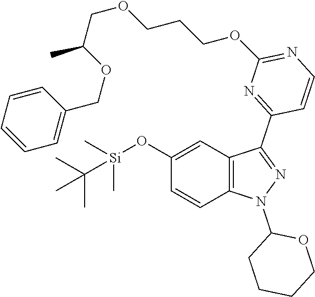 [SMILES: C[C@@H](COCCCOc1nccc(-c2nn(C3CCCCO3)c3ccc(O[Si](C)(C)C(C)(C)C)cc23)n1)OCc1ccccc1]

To a cooled solution of 3-[(2S)-2-benzyloxypropoxy]propan-1-ol (22 mg, 0.1 mmol) in THF (0.5 mL) at 0° C. was added NaH (60% dispersion in mineral oil) (5 mg, 0.110 mmol). The reaction mixture was stirred at RT for 15 min. The mixture was cooled to 0° C. and a solution of tert-butyl-dimethyl-[3-(2-methylsulfonylpyrimidin-4-yl)-1-tetrahydropyran-2-yl-indazol-5-yl] oxy-silane (49 mg, 0.1 mmol) in THF (0.5 mL) was added. The reaction mixture was stirred at 0° C. for 5 min and at RT 16 h. Saturated aqueous ammonium chloride was added, the mixture was poured in ethyl acetate and the two layers were separated. The aqueous layer was extracted with ethyl acetate. The organic layer was washed with brine, dried over anhydrous sodium sulfate, filtered and evaporated under reduced pressure to afford [3-[2-[3-[(2S)-2-benzyloxypropoxy]propoxy]pyrimidin-4-yl]-1-tetrahydropyran-2-yl-indazol-5-yl]oxy-tert-butyl-dimethyl-silane as a pale yellow oil.

LCMS method I: [M+H]⁺=655.5, t_{R}=3.81 min

### Preparation of Intermediate 70: (2S)-1-[3-[4-[5-[tert-butyl(dimethyl)silyl]oxy-1-tetrahydropyran-2-yl-indazol-3-yl]pyrimidin-2-yl]oxypropoxy]propan-2-ol

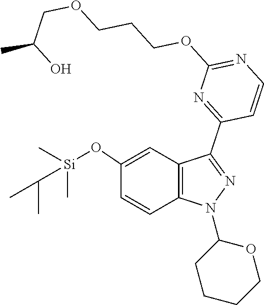 [SMILES: CC(C)[Si](C)(C)Oc1ccc2c(c1)c(-c1ccnc(OCCCOC[C@H](C)O)n1)nn2C1CCCCO1]

To a solution of [3-[2-[3-[(2S)-2-benzyloxypropoxy]propoxy]pyrimidin-4-yl]-1-tetrahydro pyran-2-yl-indazol-5-yl]oxy-tert-butyl-dimethyl-silane (63 mg, 0.1 mmol) in ethanol (2 mL) under nitrogen was added Pd/C 10% wt. (10 mg). The reaction mixture stirred at RT under hydrogen atmosphere for 16 h. The mixture was heated to 50° C. for 5 h. The reaction mixture was filtered over a pad of Celite, washed with ethanol and the solvent was evaporated under reduced pressure to afford (2S)-1-[3-[4-[5-[tert-butyl(dimethyl)silyl]oxy-1-tetrahydropyran-2-yl-indazol-3-yl]pyrimidin-2-yl]oxypropoxy]propan-2-ol as a transparent oil which was used in the next step without further purification.

LCMS method F: [M+H]⁺=543.4, t_{R}=3.56 min

### Preparation of Intermediate 71: [(1S)-2-[3-[4-[5-[tert-butyl(dimethyl)silyl]oxy-1-tetrahydropyran-2-yl-indazol-3-yl]pyrimidin-2-yl]oxypropoxy]-1-methyl-ethyl] methanesulfonate

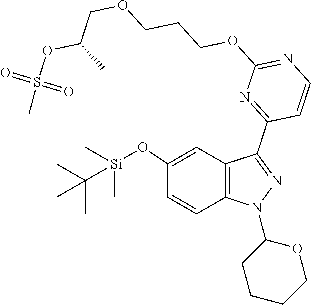 [SMILES: C[C@@H](COCCCOc1nccc(-c2nn(C3CCCCO3)c3ccc(O[Si](C)(C)C(C)(C)C)cc23)n1)OS(C)(=O)=O]

To solution of (2S)-1-[3-[4-[5-[tert-butyl(dimethyl)silyl]oxy-1-tetrahydropyran-2-yl-indazol-3-yl]pyrimidin-2-yl]oxypropoxy]propan-2-ol (54 mg, 0.1 mmol) and DIPEA (26 μL, 0.150 mmol) in dichloromethane (2 mL) at 0° C. was added methanesulfonyl chloride (9 μL, 0.120 mmol). The reaction mixture was stirred at 0° C. for 5 min then at RT for 2 h. Saturated aqueous ammonium chloride was added and the layers were separated. The aqueous layer was extracted with dichloromethane and the organic layer was washed with brine, dried over anhydrous sodium sulfate, filtered and evaporated under reduced pressure to afford [(1S)-2-[3-[4-[5-[tert-butyl(dimethyl)silyl]oxy-1-tetrahydropyran-2-yl-indazol-3-yl]pyrimidin-2-yl]oxypropoxy]-1-methyl-ethyl]methanesulfonate as a pale yellow oil which was used in the next step without further purification.

LCMS method I: [M+H]⁺=621.4, t_{R}=3.15 min

### Preparation of Intermediate 72: [(1S)-2-[3-[4-(5-hydroxy-1-tetrahydropyran-2-yl-indazol-3-yl)pyrimidin-2-yl]oxypropoxy]-1-methyl-ethyl]methanesulfonate

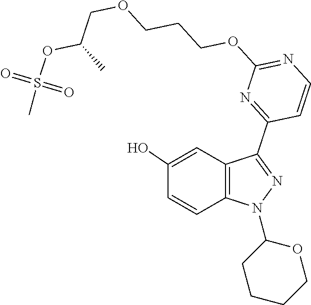 [SMILES: C[C@@H](COCCCOc1nccc(-c2nn(C3CCCCO3)c3ccc(O)cc23)n1)OS(C)(=O)=O]

To a solution of [(1S)-2-[3-[4-[5-[tert-butyl(dimethyl)silyl]oxy-1-tetrahydropyran-2-yl-indazol-3-yl]pyrimidin-2-yl]oxypropoxy]-1-methyl-ethyl] methanesulfonate (56 mg, 0.09 mmol) in THF (2 mL) at 0° C. was added TBAF (1.0 M solution in THF) (0.11 mL, 0.11 mmol). The reaction mixture was stirred at 0° C. for 5 min and at RT for 30 min. Saturated aqueous ammonium chloride and ethyl acetate were added and the two layers were separated. The aqueous layer was extracted with ethyl acetate. The organic layer was washed with brine, dried over anhydrous sodium sulfate, filtered and evaporated under reduced pressure to afford [(1S)-2-[3-[4-(5-hydroxy-1-tetrahydropyran-2-yl-indazol-3-yl)pyrimidin-2-yl]oxypropoxy]-1-methyl-ethyl]methanesulfonate as a pale yellow oil which was used in the next step without further purification.

LCMS method F: [M+H]⁺=507.3, t_{R}=2.53 min

### Preparation of Intermediate 73: (13R)-13-methyl-19-(oxan-2-yl)-7,11,14-trioxa-5,19, 20,23-tetraazatetracyclo[13.5.2.1^{2,6}.0^{18,21}]tricosa-1(20),2(23),3,5,15(22),16,18(21)-heptaene

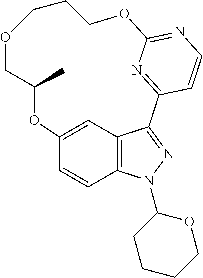 [SMILES: C[C@@H]1COCCCOc2nccc(n2)-c2nn(C3CCCCO3)c3ccc(cc23)O1]

A suspension of cesium carbonate (292 mg, 0.9 mmol) in acetonitrile (15 mL) was added at 50° C. to a solution of [(1S)-2-[3-[4-(5-hydroxy-1-tetrahydropyran-2-yl-indazol-3-yl)pyrimidin-2-yl]oxypropoxy]-1-methyl-ethyl]methanesulfonate (46 mg, 0.09 mmol) in acetonitrile (30 mL) dropwise. The reaction mixture was stirred at 50° C. for 30 min. The mixture was cooled to RT, filtered and the solvent was evaporated under reduced pressure. The residue was purified by silica gel column chromatography using cyclohexane/ethyl acetate 100/0 to 20/80 as eluent to afford (13R)-13-methyl-19-(oxan-2-yl)-7,11,14-trioxa-5,19,20,23-tetraazatetracyclo[13.5.2.1^{2,6}.0^{18,21}]tricosa-1(20),2(23),3,5,15(22),16,18(21)-heptaene as a transparent oil.

LCMS method F: [M+H]⁺=411.4, t_{R}=3.00 min

### Preparation of Example 7: (13R)-13-methyl-7,11,14-trioxa-5,19,20,23-tetraaza tetracyclo[13.5.2.1^{2,6}.0^{18,21}]tricosa-1(20),2(23),3,5,15(22),16,18(21)-heptaene

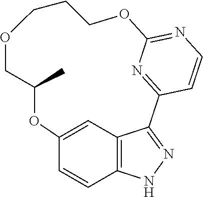 [SMILES: C[C@@H]1COCCCOc2nccc(n2)-c2n[nH]c3ccc(cc23)O1]

To a solution of (13R)-13-methyl-19-(oxan-2-yl)-7,11,14-trioxa-5,19,20,23-tetraazatetra cyclo[13.5.2.1^{2,6}.0^{18,21}]tricosa-1(20),2(23),3,5,15(22),16,18(21)-heptaene (121 mg, 0.295 mmol) in methanol (3 mL) and water (0.5 mL) was added p-toluenesulfonic acid monohydrate (281 mg, 1.475 mmol). The reaction mixture was heated to 60° C. for 16 h. The solvent was evaporated under reduced pressure and the residue was partitioned between ethyl acetate and a saturated aqueous NaHCO₃ solution. The aqueous layer was extracted with ethyl acetate. The organic layer was washed with brine, dried over anhydrous sodium sulfate, filtered and evaporated under reduced pressure. The residue was purified by silica gel column chromatography using dichloromethane/methanol 100/0 to 95/5 as eluent. The resulting solid was triturated with diisopropylether/acetonitrile (20:1 v:v), filtered and dried to afford (13R)-13-methyl-7,11,14-trioxa-5,19,20,23-tetraazatetracyclo[13.5.2.1^{2,6}.0^{18,21}]tricosa-1(20),2(23), 3,5,15(22),16,18(21)-heptaene as a solid.

LCMS method F: [M+H]⁺=327.3, t_{R}=3.13 min

LCMS method G: [M+H]⁺=327.3, t_{R}=3.15 min

¹H NMR (400 MHz, d6-DMSO) δ 13.63 (s, 1H), 8.75 (d, J=2.2 Hz, 1H), 8.59 (d, J=5.0 Hz, 1H), 7.76 (d, J=5.3 Hz, 1H), 7.51 (d, J=8.5 Hz, 1H), 7.06 (dd, J=2.4, 8.6 Hz, 1H), 5.20 (dt, J=4.5, 11.8 Hz, 1H), 4.40-4.34 (m, 2H), 3.79 (dd, J=6.4, 9.9 Hz, 1H), 3.69-3.56 (m, 2H), 3.45 (dd, J=3.7, 9.7 Hz, 1H), 2.41-2.30 (m, 1H), 1.87-1.80 (m, 1H), 1.34 (d, J=6.8 Hz, 3H) ppm.

### Example 8: (6R)-6-methyl-9,13-dioxa-4,5,18,19-tetraazatetracyclo[12.5.2.1^{2,5}.0^{17,20}] docosa-1(19),2(22),3,14(21),15,17(20)-hexaene

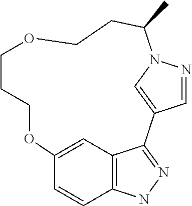 [SMILES: C[C@@H]1CCOCCCOc2ccc3[nH]nc(c3c2)-c2cnn1c2]

Example 8 is prepared according to the synthesis route described in general Scheme B.

### Preparation of Intermediate 74: ethyl (S)-3-((tert-butyldiphenylsilyl)oxy)butanoate

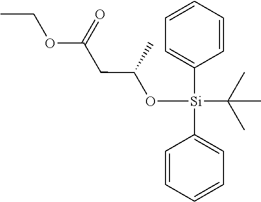 [SMILES: CCOC(=O)C[C@H](C)O[Si](c1ccccc1)(c1ccccc1)C(C)(C)C]

To a solution of ethyl (S)-3-hydroxybutanoate (5 g, 37.833 mmol) in dichloromethane (114 mL) was added imidazole (3.348 g, 49.183 mmol) and tert-butyl(chloro)diphenylsilane (11.449 mL, 45.4 mmol). The reaction mixture was stirred at RT for 3 h. The mixture was diluted with dichloromethane and saturated aqueous NaHCO₃ solution. The aqueous layer was extracted with dichloromethane. The organic layer was dried over anhydrous magnesium sulfate, filtered and evaporated under reduced pressure. The residue was purified by silica gel column chromatography using heptane/ethyl acetate 100/0 to 95/5 as eluent to afford ethyl (S)-3-((tert-butyldiphenylsilyl)oxy)butanoate as a pale yellow oil.

LCMS method B: [M+H]⁺=not detected, t_{R}=1.482 min

### Preparation of Intermediate 75: (S)-3-((tert-butyldiphenylsilyl)oxy)butan-1-ol

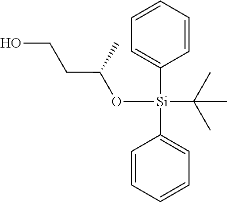 [SMILES: C[C@@H](CCO)O[Si](c1ccccc1)(c1ccccc1)C(C)(C)C]

To a solution of ethyl (S)-3-((tert-butyldiphenylsilyl)oxy)butanoate (12.85 g, 34.676 mmol) in ethanol (173 mL) and THF (173 mL) at 0° C. was added sodium borohydride (2.624 g, 69.352 mmol) and calcium dichloride (3.849 g, 34.676 mmol). The reaction mixture was stirred at RT for 20 h. The mixture was cooled at 0° C. and additional sodium borohydride (1.312 g, 34.676 mmol) was added. The reaction mixture was stirred at RT for 20 h. The reaction mixture was filtered and the filtrate was diluted with ethyl acetate and water. The aqueous layer was extracted with ethyl acetate. The organic layer was dried over anhydrous magnesium sulfate, filtered and evaporated under reduced pressure. The residue was purified by silica gel column chromatography using heptane/ethyl acetate 100/0 to 80/20 as eluent to afford (S)-3-((tert-butyldiphenylsilyl)oxy)butan-1-ol as a colorless oil.

LCMS method B: [M+H]⁺=not detected, t_{R}=1.284 min

### Preparation of Intermediate 76: (S)-3-((tert-butyldiphenylsilyl)oxy)butylmethane sulfonate

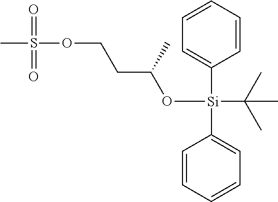 [SMILES: C[C@@H](CCOS(C)(=O)=O)O[Si](c1ccccc1)(c1ccccc1)C(C)(C)C]

To a stirred solution of (S)-3-((tert-butyldiphenylsilyl)oxy)butan-1-ol (7.75 g, 23.59 mmol) in dichloromethane (70 mL) was added triethylamine (4.932 ml, 35.385 mmol). The reaction mixture was cooled at 0° C. and methanesulfonyl chloride (2.374 mL, 30 667 mmol) was added under nitrogen atmosphere. The reaction mixture was stirred at RT for 1.5 h. The mixture was diluted with dichloromethane and 10% aqueous NaHCO₃ solution. The aqueous layer was extracted with dichloromethane. The organic layer was dried over anhydrous magnesium sulfate, filtered and evaporated under reduced pressure to afford (S)-3-((tert-butyldiphenylsilyl)oxy)butylmethanesulfonate as an orange oil which was used in the next step without purification.

LCMS method B: [M+H]⁺=not detected, t_{R}=1.35 min

### Preparation of Intermediate 77: [(1S)-3-(3-benzyloxypropoxy)-1-methyl-propoxy]-tert-butyl-diphenyl-silane

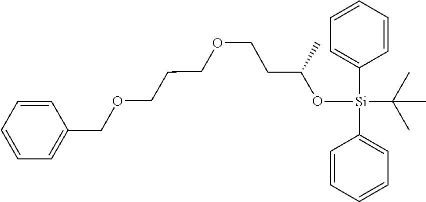 [SMILES: C[C@@H](CCOCCCOCc1ccccc1)O[Si](c1ccccc1)(c1ccccc1)C(C)(C)C]

To a solution of 3-(benzyloxy)propan-1-ol (3.26 g, 19.613 mmol) in dry THF (88 mL) at 0° C. under nitrogen atmosphere was added sodium hydride (60% dispersion in mineral oil) (2.354 g, 58.839 mmol). The reaction mixture was stirred at 0° C. for 1 h. (S)-3-((tert-butyldiphenylsilyl)oxy)butyl methanesulfonate (10.98 g, 23.536 mmol) in dry THF (10 mL) was added and the reaction mixture was stirred at 80° C. for 16 h. The mixture was diluted with ethyl acetate and water. The aqueous layer was extracted with ethyl acetate. The organic layer was dried over anhydrous magnesium sulfate, filtered and evaporated under reduced pressure. The residue was purified by silica gel column chromatography using heptane/ethyl acetate 100/0 to 98.5/1.5 as eluent to afford [(1S)-3-(3-benzyloxypropoxy)-1-methyl-propoxy]-tert-butyl-diphenyl-silane as a colorless oil.

LCMS method B: [M+H]⁺=not detected, t_{R}=1.667 min

### Preparation of Intermediate 78: (S)-4-(3-(benzyloxy)propoxy)butan-2-ol

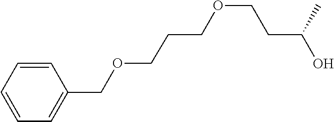 [SMILES: C[C@H](O)CCOCCCOCc1ccccc1]

To a solution of [(1S)-3-(3-benzyloxypropoxy)-1-methyl-propoxy]-tert-butyl-diphenyl-silane (4.28 g, 8.978 mmol) in THF (45 mL) at 0° C. was added TBAF (1 M solution in THF) (13.467 mL, 13.467 mmol). The reaction mixture was stirred at RT for 20 h. The reaction mixture was cooled at 0° C. then diluted with ethyl acetate, saturated aqueous NaHCO₃ solution. The aqueous layer was extracted with ethyl acetate. The organic layer was dried over anhydrous magnesium sulfate, filtered and evaporated under reduced pressure. The residue was purified by silica gel column chromatography using heptane/ethyl acetate 100/0 to 60/40 as eluent to afford (S)-4-(3-(benzyloxy)propoxy)butan-2-ol as a colorless oil.

LCMS method B: [M+H]⁺=239.1, t_{R}=0.720 min

### Preparation of Intermediate 79: (S)-4-(3-(benzyloxy)propoxy)butan-2-yl methane sulfonate

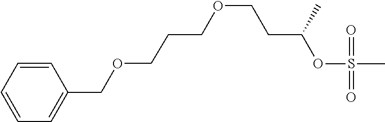 [SMILES: C[C@@H](CCOCCCOCc1ccccc1)OS(C)(=O)=O]

To a stirred solution of (S)-4-(3-(benzyloxy)propoxy)butan-2-ol (1.85 g, 7.762 mmol) in dichloromethane (23 mL) was added triethylamine (1.623 mL, 11.643 mmol). The reaction mixture was cooled at 0° C. and methanesulfonyl chloride (781 □L, 10.091 mmol) was added under nitrogen atmosphere. The reaction mixture was stirred at RT for 1 h. The mixture was diluted with dichloromethane and an 10% aqueous NaHCO₃ solution. The aqueous layer was extracted with dichloromethane. The organic layer was dried over anhydrous magnesium sulfate, filtered and evaporated under reduced pressure to afford (S)-4-(3-(benzyloxy)propoxy)butan-2-yl methanesulfonate as a yellow oil which was used in the next step without purification.

LCMS method B: [M+H]⁺=317.0, t_{R}=0.899 min

### Preparation of Intermediate 80: 1-[(1R)-3-(3-benzyloxypropoxy)-1-methyl-propyl]-4-(4,4,5,5-tetramethyl-1,3,2-dioxaborolan-2-yl)pyrazole

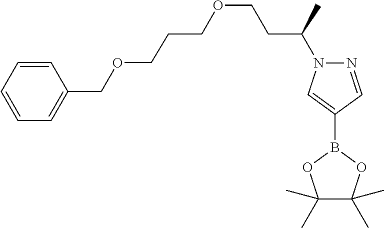 [SMILES: C[C@H](CCOCCCOCc1ccccc1)n1cc(B2OC(C)(C)C(C)(C)O2)cn1]

A mixture of (S)-4-(3-(benzyloxy)propoxy)butan-2-yl methanesulfonate (2.66 g, 7.762 mmol), 4-(4,4,5,5-tetramethyl-1,3,2-dioxaborolan-2-yl)-1H-pyrazole (1.657 g, 8.538 mmol) and cesium carbonate (3.035 g, 9.314 mmol) in acetonitrile (44 mL) was stirred at 70° C. for 42 h. Additional cesium carbonate (2.529 g, 7.762 mmol) was added and the reaction mixture was stirred at 70° C. for 22 h. Additional cesium carbonate (1.265 g, 3.881 mmol) was added and the reaction mixture was stirred at 70° C. for 18 h. Additional 4-(4,4,5,5-tetramethyl-1,3,2-dioxaborolan-2-yl)-1H-pyrazole (452 mg, 2.329 mmol) was added and the mixture was stirred at 80° C. for 6 h. The mixture was diluted with acetonitrile and filtered. The filtrate was evaporated under reduced pressure and the residue was purified by silica gel column chromatography using dichloromethane/ethyl acetate 98/2 to 87/13 as eluent to afford 1-[(1R)-3-(3-benzyloxypropoxy)-1-methyl-propyl]-4-(4,4,5,5-tetramethyl-1,3,2-dioxaborolan-2-yl)pyrazole as a colorless oil.

LCMS method E: [M+H]⁺=415.3, t_{R}=4.124 min

### Preparation of Intermediate 81: [3-[1-[(1R)-3-(3-benzyloxypropoxy)-1-methyl-propyl]pyrazol-4-yl]-1-tetrahydropyran-2-yl-indazol-5-yl]oxy-tert-butyl-dimethyl-silane

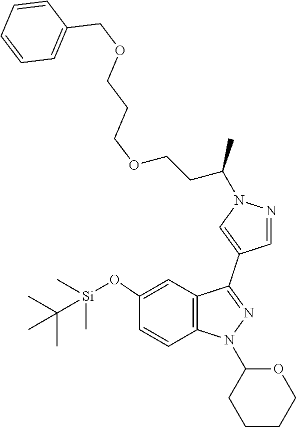 [SMILES: C[C@H](CCOCCCOCc1ccccc1)n1cc(-c2nn(C3CCCCO3)c3ccc(O[Si](C)(C)C(C)(C)C)cc23)cn1]

To a solution of tert-butyl-(3-iodo-1-tetrahydropyran-2-yl-indazol-5-yl)oxy-dimethyl-silane (1.5 g, 3.27 mmol) in dioxane (15 mL) and water (1.5 mL) at RT was added 1-[(1R)-3-(3-benzyloxypropoxy)-1-methyl-propyl]-4-(4,4,5,5-tetramethyl-1,3,2-dioxaborolan-2-yl) pyrazole (1.491 g, 3.60 mmol), potassium phosphate tribasic (2.084 g, 9.82 mmol), XPhos (156 mg, 0.33 mmol) and Pd(PPh₃)₄ (189 mg, 0.16 mmol). The reaction mixture was stirred under microwave irradiations at 120° C. for 10 h. The mixture was diluted with brine and extracted with ethyl acetate. The organic layer was dried over anhydrous sodium sulfate and the solvent was evaporated under reduced pressure. The residue was purified by silica gel column chromatography using cyclohexane/(ethyl acetate/ethanol (3/1)) 100/0 to 90/10 as eluent to afford [3-[1-[(1R)-3-(3-benzyloxypropoxy)-1-methyl-propyl]pyrazol-4-yl]-1-tetrahydropyran-2-yl-indazol-5-yl]oxy-tert-butyl-dimethyl-silane as a colorless oil.

LCMS method J: [M+H]⁺=619.4, t_{R}=5.26 min

### Preparation of Intermediate 82: 3-[(3R)-3-[4-[5-[tert-butyl(dimethyl)silyl]oxy-1-tetrahydropyran-2-yl-indazol-3-yl]pyrazol-1-yl]butoxy]propan-1-ol

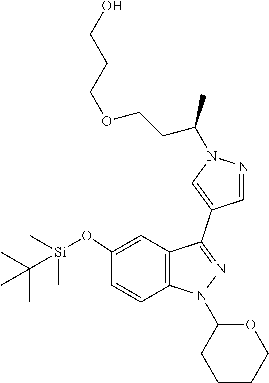 [SMILES: C[C@H](CCOCCCO)n1cc(-c2nn(C3CCCCO3)c3ccc(O[Si](C)(C)C(C)(C)C)cc23)cn1]

To a solution of [3-[1-[(1R)-3-(3-benzyloxypropoxy)-1-methyl-propyl]pyrazol-4-yl]-1-tetrahydropyran-2-yl-indazol-5-yl]oxy-tert-butyl-dimethyl-silane (520 mg, 0.84 mmol) in methanol (20 mL) at RT was added palladium hydroxide (52 mg). The reaction mixture was stirred under hydrogen atmosphere at RT overnight. The reaction mixture was filtered and the filtrate was evaporated under reduced pressure. The residue was purified by silica gel column chromatography using dichloromethane/ethyl acetate 100/0 to 40/60 as eluent to afford 3-[(3R)-3-[4-[5-[tert-butyl(dimethyl)silyl]oxy-1-tetrahydropyran-2-yl-indazol-3-yl]pyrazol-1-yl]butoxy]propan-1-ol as a colorless oil.

LCMS method F: [M+H]⁺=529.4, t_{R}=3.34 min

### Preparation of Intermediate 83: 3-[(3R)-3-[4-[5-[tert-butyl(dimethyl)silyl]oxy-1-tetrahydropyran-2-yl-indazol-3-yl]pyrazol-1-yl]butoxy]propyl methanesulfonate

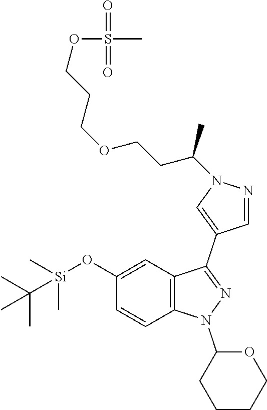 [SMILES: C[C@H](CCOCCCOS(C)(=O)=O)n1cc(-c2nn(C3CCCCO3)c3ccc(O[Si](C)(C)C(C)(C)C)cc23)cn1]

To a solution of 3-[(3R)-3-[4-[5-[tert-butyl(dimethyl)silyl]oxy-1-tetrahydropyran-2-yl-indazol-3-yl]pyrazol-1-yl]butoxy]propan-1-ol (140 mg, 0.26 mmol) and triethylamine (74 μL, 0.53 mmol) in dichloromethane (4 mL) at 0° C. was added methanesulfonyl chloride (27 μL, 0.34 mmol) in dichloromethane (1 mL). The reaction mixture was stirred at RT for 1 h. The mixture was diluted with brine and extracted with dichloromethane. The organic layer was dried over anhydrous sodium sulfate, filtered and evaporated under reduced pressure to afford 3-[(3R)-3-[4-[5-[tert-butyl(dimethyl)silyl]oxy-1-tetrahydropyran-2-yl-indazol-3-yl]pyrazol-1-yl]butoxy]propylmethanesulfonate as a yellow oil which was used in the next step without further purification.

LCMS method F: [M+H]⁺=607.3, t_{R}=3.48 min

### Preparation of Intermediate 84: 3-[(3R)-3-[4-(5-hydroxy-1-tetrahydropyran-2-yl-indazol-3-yl)pyrazol-1-yl]butoxy]propylmethanesulfonate

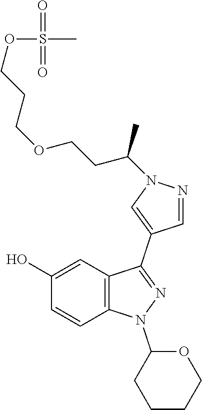 [SMILES: C[C@H](CCOCCCOS(C)(=O)=O)n1cc(-c2nn(C3CCCCO3)c3ccc(O)cc23)cn1]

To a solution of 3-[(3R)-3-[4-[5-[tert-butyl(dimethyl)silyl]oxy-1-tetrahydropyran-2-yl-indazol-3-yl]pyrazol-1-yl]butoxy]propyl methanesulfonate (160 mg, 0.26 mmol) in THF (5 mL) at −15° C. was added TBAF (1M solution in THF) (290 μL, 0.29 mmol). The reaction mixture was stirred at −15° C. for 10 min. The reaction mixture was diluted with a saturated aqueous ammonium chloride solution and extracted with ethyl acetate. The organic layer was dried over anhydrous sodium sulfate, filtered and evaporated under reduced pressure to afford 3-[(3R)-3-[4-(5-hydroxy-1-tetrahydropyran-2-yl-indazol-3-yl)pyrazol-1-yl]butoxy]propyl methanesulfonate as a yellow oil which was used in the next step without further purification.

LCMS method F: [M+H]⁺=493.2, t_{R}=2.37 min

### Preparation of Intermediate 85: (6R)-6-methyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene

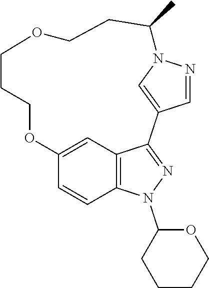 [SMILES: C[C@@H]1CCOCCCOc2ccc3c(c2)c(nn3C2CCCCO2)-c2cnn1c2]

To a suspension of cesium carbonate (179 mg, 0.55 mmol) in anhydrous DMF (45 mL) at 80° C. was added dropwise 3-[(3R)-3-[4-(5-hydroxy-1-tetrahydropyran-2-yl-indazol-3-yl)pyrazol-1-yl]butoxy]propylmethanesulfonate (90 mg, 0.18 mmol) in DMF (45 mL). The reaction mixture was stirred at 80° C. for 30 min. The reaction mixture was concentrated under reduced pressure, diluted with brine and extracted with ethyl acetate. The organic layer was dried over anhydrous sodium sulfate, filtered and evaporated under reduced pressure. The residue was purified by silica gel column chromatography using dichloromethane/ethyl/acetate 100/0 to 60/40 as eluent to afford (6R)-6-methyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene as a yellow oil.

LCMS method F: [M+H]⁺=397.4, t_{R}=2.62 min

### Preparation of Example 8: (6R)-6-methyl-9,13-dioxa-4,5,18,19-tetraazatetracyclo [12.5.2.1^{2,5}.0^{17,20}]docosa-1(19),2(22),3,14(21),15,17(20)-hexaene

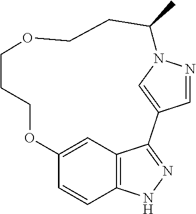 [SMILES: C[C@@H]1CCOCCCOc2ccc3[nH]nc(c3c2)-c2cnn1c2]

To a solution of (6R)-6-methyl-18-(oxan-2-yl)-9,13-dioxa-4,5,18,19-tetraazatetracyclo [12.5.2.1^{2,5}.0^{17,20}]docosa-1(19),2(22),3,14(21),15,17(20)-hexaene (40 mg, 0.10 mmol) in methanol (3.5 mL) and water (0.5 mL) was added p-toluenesulfonic acid monohydrate (96 mg, 0.50 mmol). The reaction mixture was stirred at 65° C. for 2 h. The reaction mixture was concentrated under reduced pressure and the residue was neutralized by slow addition of a saturated aqueous NaHCO₃ solution. The aqueous phase was extracted with ethyl acetate. The organic layer was washed with brine, dried over anhydrous sodium sulfate, filtered and evaporated under reduced pressure. The resulting solid was recrystallized with acetonitrile to afford (6R)-6-methyl-9,13-dioxa-4,5,18,19-tetraazatetracyclo[12.5.2.1^{2,5}.0^{17,20}]docosa-1(19), 2(22),3,14(21),15,17(20)-hexaene as a solid.

LCMS method F: [M+H]⁺=313.3, t_{R}=2.04 min

LCMS method G: [M+H]⁺=313.3, t_{R}=2.05 min

¹H NMR (400 MHz, d6-DMSO) δ 12.68 (1H, s), 8.59 (1H, s), 7.68 (1H, d, J=0.6 Hz), 7.46-7.37 (2H, m), 6.94 (1H, dd, J=2.5, 8.9 Hz), 4.60-4.53 (1H, m), 4.36-4.29 (2H, m), 3.56-3.43 (4H, m), 2.24-2.19 (2H, m), 2.08-2.00 (2H, m), 1.53 (3H, d, J=6.8 Hz) ppm.

### Example 9: (7S,13S)-7,13-dimethyl-8,11,14-trioxa-4,19,20-triazatetracyclo[13.5.2.1^{2,6}.0^{18,21}]tricosa-1(20),2(23),3,5,15(22),16,18(21)-heptaene

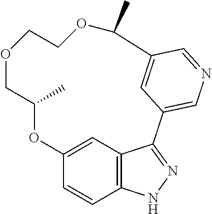 [SMILES: C[C@@H]1OCCOC[C@H](C)Oc2ccc3[nH]nc(c3c2)-c2cncc1c2]

Example 9 is prepared according to the synthesis route described in general Scheme D.

### Preparation of Intermediate 86: methyl (2R)-2-benzyloxypropanoate

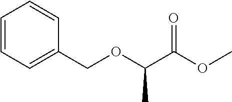

To a solution of methyl (R)-(+)-lactate (5 g, 48.07 mmol) in dichloromethane (24 mL) at RT was added sequentially a solution of benzyl 2,2,2-trichloroacetimidate (17.8 mL, 96.14 mmol) in pentane (145 mL) and triflic acid (210 μL, 2.40 mmol). The suspension was stirred for 16 h at RT. The reaction mixture was filtered off and rinsed with hexane. The filtrate was then washed with a saturated aqueous NaHCO₃ solution, the layers were separated and the aqueous layer was extracted with hexane. The combined organic layers were dried over anhydrous magnesium sulfate, filtered and evaporated under reduced pressure. The residue was purified by silica gel column chromatography using cyclohexane/ethyl acetate 100/0 to 70/30 as eluent to afford methyl (2R)-2-benzyloxypropanoate as a colorless oil.

¹H NMR (400 MHz, CDCl₃) 7.42-7.27 (5H, m), 4.72 (1H, J_{AB}=11.6 Hz), 4.48 (1H, AB syst, J_{AB}=11.6 Hz), 4.10 (1H, q, J=6.8 Hz), 3.78 (3H, s), 1.46 (3H, d, J=7.2 Hz) ppm.

### Preparation of Intermediate 87: (2R)-2-benzyloxypropan-1-ol

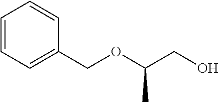

To a suspension of lithium borohydride (959 mg, 43.60 mmol) in dry THF (50 mL) at −17° C. was added dropwise a solution of methyl (2R)-2-benzyloxypropanoate (8.056 g, 2.57 mmol) in dry THF (30 mL). The reaction mixture was stirred at −12° C. for 2 h then at RT overnight. Ice-cold water was added and the aqueous layer was extracted with diethyl ether. The combined organic layers were washed with a saturated aqueous NaHCO₃ solution, a saturated aqueous ammonium chloride solution and brine, dried over anhydrous magnesium sulfate, filtered and evaporated under reduced pressure to afford (2R)-2-benzyloxypropan-1-ol as a colorless oil.

¹H NMR (400 MHz, CDCl₃) 7.43-7.27 (5H, m), 4.68 (1H, J_{AB}=11.2 Hz), 4.51 (1H, AB syst, J_{AB}=11.2 Hz), 3.75-3.68 (1H, m), 3.66-3.60 (1H, m), 3.56-3.50 (1H, m), 2.13 (1H, br. s, OH), 1.20 (3H, d, J=6.4 Hz) ppm.

### Preparation of Intermediate 88: (2R)-2-(benzyloxy)propyl-4-methylbenzene-1-sulfonate

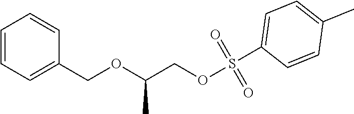 [SMILES: Cc1ccc(S(=O)(=O)OC[C@@H](C)OCc2ccccc2)cc1]

To a suspension (2R)-2-(benzyloxy)propan-1-ol (2.7 g, 16.24 mmol) in pyridine (27 mL) at 0° C. was added p-toluenesulfonyl chloride (3.407 g, 17.87 mmol). The reaction mixture was stirred at RT overnight. The mixture was diluted in ethyl acetate washed with water and brine. The organic layer was dried over anhydrous sodium sulfate, filtered and evaporated under reduced pressure. The residue was purified by silica gel column chromatography using cyclohexane/ethyl acetate 100/0 to 90/10 as eluent to afford (2R)-2-(benzyloxy)propyl 4-methylbenzene-1-sulfonate as a yellow oil.

LCMS method F: [M+H]⁺=321.1, t_{R}=2.95 min

### Preparation of Intermediate 89: 1-(5-bromopyridin-3-yl)ethanol

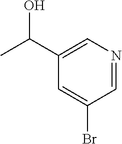

A solution of (S)-(−)-α,α-diphenyl-2-pyrrolidineMeOH (253 mg, 1.00 mmol) and trimethyl borate (134 μL, 1.20 mmol) in tetrahydrofuran (20 mL) was stirred at RT for 1 h. Borane tetrahydrofuran complex 1M solution (20 mL, 20 mmol) was added in one portion and the reaction mixture was cooled to 0° C. A solution of 1-(5-bromopyridin-3-yl)ethan-1-one (2 g, 10 mmol) in THF (20 mL) was added dropwise at 0° C. The reaction mixture was stirred at RT overnight. 1M aqueous hydrochloric acid solution (9 mL) was added and the reaction mixture was stirred at RT for 2 h. The solvent was evaporated under reduced pressure and the resulting solution was basified to pH 11 with aqueous ammonia, extracted with ethyl acetate. The combined organic layers were washed with brine, dried over anhydrous magnesium sulfate, filtered and evaporated under reduced pressure to afford 1-(5-bromopyridin-3-yl)ethanol as a yellow oil.

LCMS method F: [M+H]⁺=202, t_{R}=1.46 min

### Preparation of Intermediates 90A and 90B: (1R)-1-(5-bromopyridin-3-yl)ethan-1-ol and (1S)-1-(5-bromopyridin-3-yl)ethan-1-ol

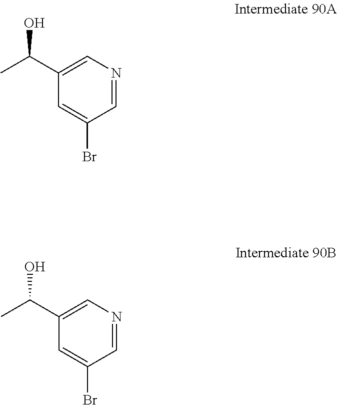

The racemic mixture of 1-(5-bromopyridin-3-yl)ethan-1-ol (1.664 g, 8.24 mmol) was purified by chiral HPLC using the following conditions:

Analyses were carried out on Chiralpak IA 20×250 mm 5 μm [C7/EtOH]⁺0.1% DEA [95/5] 30 min with a flow rate of 19 mL/min at RT. The desired fractions were collected and the solvent was removed under reduced pressure to afford (1R)-1-(5-bromopyridin-3-yl)ethan-1-ol as a yellow oil and (1S)-1-(5-bromopyridin-3-yl)ethan-1-ol as a yellow oil.

Intermediate 90A: LCMS method F: [M+H]⁺=202.0-204.0, t_{R}=1.49 min

Intermediate 90B: LCMS method F: [M+H]⁺=202.1-204.1, t_{R}=1.37 min

### Preparation of Intermediate 91: 3-bromo-5-[(1S)-1-[2-(oxan-2-yloxy)ethoxy]ethyl] pyridine

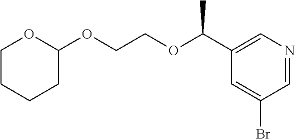 [SMILES: C[C@H](OCCOC1CCCCO1)c1cncc(Br)c1]

To a solution of (1S)-1-(5-bromopyridin-3-yl)ethan-1-ol (570 mg, 2.82 mmol) in DMF (28 mL) at 0° C. was added sodium hydride (60% dispersion in mineral oil) (169 mg, 4.23 mmol) and 2-(2-bromoethoxy)oxane (852 μL, 5.64 mmol). The reaction mixture was stirred at RT overnight. The mixture was diluted with ethyl acetate washed with water then brine. The organic layer was dried over anhydrous sodium sulfate, filtered and evaporated under reduced pressure. The residue was purified by silica gel column chromatography using cyclohexane/ethyl acetate 100/0 to 80/20 as eluent to afford 3-bromo-5-[(1S)-1-[2-(oxan-2-yloxy)ethoxy]ethyl]pyridine as a yellow oil.

LCMS method F: [M+H]⁺=330.1-332.1, t_{R}=2.52 min

### Preparation of Intermediate 92: 2-[(1S)-1-(5-bromopyridin-3-yl)ethoxy]ethan-1-ol

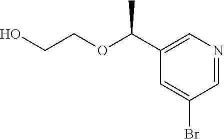

To a solution of 3-bromo-5-[(1S)-1-[2-(oxan-2-yloxy)ethoxy]ethyl]pyridine (801 mg, 2.43 mmol) in methanol (40 mL) was added 1M aqueous hydrochloric acid solution (4.851 mL, 4.85 mmol). The reaction mixture was stirred at RT for 2 h. The pH of the solution was adjusted 10 by the addition of 1M aqueous sodium hydroxide solution. The aqueous layer was extracted with ethyl acetate. The combined organic layers were dried over anhydrous sodium sulfate, filtered and evaporated under reduced pressure to afford 2-[(1S)-1-(5-bromopyridin-3-yl)ethoxy]ethan-1-ol as a yellow oil.

LCMS method F: [M+H]⁺=246.1-248.1, t_{R}=1.64 min

### Preparation of Intermediate 93: 3-[(1S)-1-{2-[(2R)-2-(benzyloxy)propoxy]ethoxy}ethyl]-5-bromopyridine

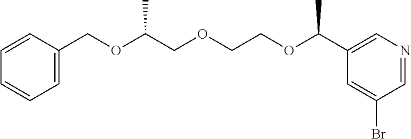 [SMILES: C[C@H](COCCO[C@@H](C)c1cncc(Br)c1)OCc1ccccc1]

To a solution of 2-[(1S)-1-(5-bromopyridin-3-yl)ethoxy]ethan-1-ol (582 mg, 2.36 mmol) in DMF (7 mL) at 0° C. was added sodium hydride (60% dispersion in mineral oil) (142 mg, 3.55 mmol). After 10 min, (2R)-2-(benzyloxy)propyl 4-methylbenzene-1-sulfonate (1.515 g, 4.73 mmol) in DMF (3 mL) was added. The reaction mixture was stirred at RT overnight. The mixture was diluted with ethyl acetate and washed with water and brine. The organic layer was dried over anhydrous sodium sulfate, filtered and evaporated under reduced pressure. The residue was purified by silica gel column chromatography using cyclohexane/ethyl acetate 100/0 to 80/20 as eluent to afford 3-[(1S)-1-{2-[(2R)-2-(benzyloxy)propoxy]ethoxy}ethyl]-5-bromopyridine as a colorless oil.

LCMS method F: [M+H]⁺=394.1-396.1, t_{R}=2.94 min

### Preparation of Intermediate 94: [3-[5-[(1S)-1-[2-[(2R)-2-benzyloxypropoxy]ethoxy] ethyl]-3-pyridyl]-1-tetrahydropyran-2-yl-indazol-5-yl]oxy-tert-butyl-dimethyl-silane

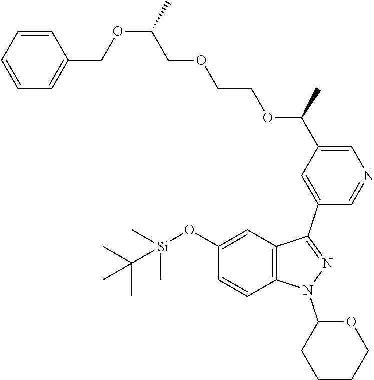 [SMILES: C[C@H](COCCO[C@@H](C)c1cncc(-c2nn(C3CCCCO3)c3ccc(O[Si](C)(C)C(C)(C)C)cc23)c1)OCc1ccccc1]

To a mixture of 3-[(1S)-1-{2-[(2R)-2-(benzyloxy)propoxy]ethoxy}ethyl]-5-bromopyridine (627 mg, 1.59 mmol), 5-[(tert-butyldimethylsilyl)oxy]-1-(oxan-2-yl)-3-(4,4,5,5-tetramethyl-1,3,2-dioxaborolan-2-yl)-1H-indazole (948 mg, 2.07 mmol), XPhos (76 mg, 0.16 mmol), and potassium phosphate tribasic (1.013 g, 4.77 mmol) in dioxane (15.1 mL) and water (1.7 mL) stirred under argon for 15 min was added tetrakis(triphenylphosphine)palladium(O) (92 mg, 0.08 mmol). The mixture was heated at 90° C. for 30 min under microwave irradiations. The reaction mixture was cooled and filtered over celite. The solvent was evaporated under reduced pressure. The residue was purified by silica gel column chromatography using cyclohexane/ethyl acetate 100/0 to 70/30 as eluent afford [3-[5-[(1S)-1-[2-[(2R)-2-benzyloxy propoxy]ethoxy]ethyl]-3-pyridyl]-1-tetrahydropyran-2-yl-indazol-5-yl]oxy-tert-butyl-dimethyl-silane as a pale yellow oil.

LCMS method F: [M+H]⁺=646.4, t_{R}=3.88 min

### Preparation of Intermediate 95: (2R)-1-{2-[(1S)-1-(5-{5-[(tert-butyldimethylsilyl) oxy]-1-(oxan-2-yl)-1H-indazol-3-yl}pyridin-3-yl)ethoxy]ethoxy}propan-2-ol

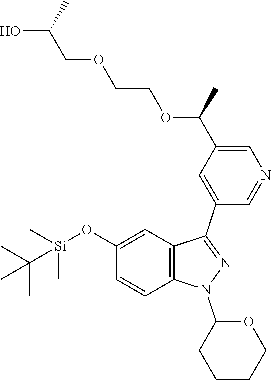 [SMILES: C[C@H](OCCOC[C@@H](C)O)c1cncc(-c2nn(C3CCCCO3)c3ccc(O[Si](C)(C)C(C)(C)C)cc23)c1]

To a solution of [3-[5-[(1S)-1-[2-[(2R)-2-benzyloxypropoxy]ethoxy]ethyl]-3-pyridyl]-1-tetrahydropyran-2-yl-indazol-5-yl]oxy-tert-butyl-dimethyl-silane (963 mg, 1.49 mmol) in ethanol (20 mL) under argon was added palladium hydroxide on charcoal 10% (96 mg, 0.90 mmol). The reaction mixture was stirred under hydrogen atmosphere at RT overnight then under hydrogen atmosphere at 50° C. overnight. The reaction mixture was filtered and the solvent was evaporated under reduced pressure to afford (2R)-1-{2-[(1S)-1-(5-{5-[(tert-butyldimethylsilyl)oxy]-1-(oxan-2-yl)-1H-indazol-3-yl}pyridin-3-yl)ethoxy]ethoxy}propan-2-ol as a colorless oil.

LCMS method F: [M+H]⁺=556.3, t_{R}=3.25 min

### Preparation of Intermediate 96: [(1R)-2-[2-[(1S)-1-[5-[5-[tert-butyl(dimethyl)silyl] oxy-1-tetrahydropyran-2-yl-indazol-3-yl]-3-pyridyl]ethoxy]ethoxy]-1-methyl-ethyl] methanesulfonate

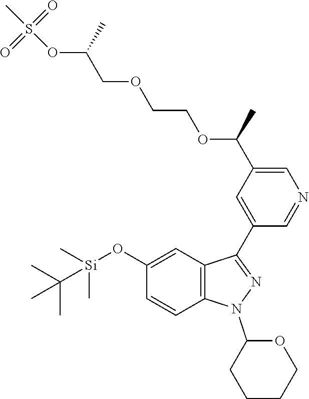 [SMILES: C[C@H](COCCO[C@@H](C)c1cncc(-c2nn(C3CCCCO3)c3ccc(O[Si](C)(C)C(C)(C)C)cc23)c1)OS(C)(=O)=O]

To a suspension (2R)-1-{2-[(1S)-1-(5-{5-[(tert-butyldimethylsilyl)oxy]-1-(oxan-2-yl)-1H-indazol-3-yl}pyridin-3-yl)ethoxy]ethoxy}propan-2-ol (500 mg, 0.90 mmol) in dichloro methane (10 mL) at 0° C. was added triethylamine (188 μL, 1.35 mmol) and methanesulfonyl chloride (77 μL, 0.99 mmol). The reaction mixture was stirred at RT for 30 min. The mixture was diluted with water and the aqueous layer was extracted with dichloromethane. The organic layer was dried over anhydrous sodium sulfate, filtered and evaporated under reduced pressure to afford [(1R)-2-[2-[(1S)-1-[5-[5-[tert-butyl(dimethyl)silyl]oxy-1-tetrahydropyran-2-yl-indazol-3-yl]-3-pyridyl]ethoxy]ethoxy]-1-methyl-ethyl] methanesulfonate as a yellow oil.

LCMS method F: [M+H]⁺=634.4, t_{R}=3.48 min

### Preparation of Intermediate 97: [(1R)-2-[2-[(1S)-1-[5-(5-hydroxy-1-tetrahydropyran-2-yl-indazol-3-yl)-3-pyridyl]ethoxy]ethoxy]-1-methyl-ethyl]methanesulfonate

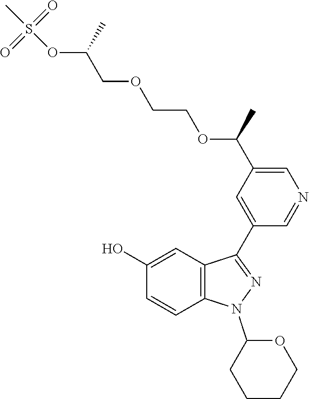 [SMILES: C[C@H](COCCO[C@@H](C)c1cncc(-c2nn(C3CCCCO3)c3ccc(O)cc23)c1)OS(C)(=O)=O]

To a solution of [(1R)-2-[2-[(1S)-1-[5-[5-[tert-butyl(dimethyl)silyl]oxy-1-tetrahydropyran-2-yl-indazol-3-yl]-3-pyridyl]ethoxy]ethoxy]-1-methyl-ethyl]methanesulfonate (570 mg, 0.90 mmol) in THF (8 mL) at 0° C. was added TBAF (1M solution in THF) (1.798 mL, 1.80 mmol). The reaction mixture was stirred at 0° C. for 30 min. The reaction mixture was diluted with saturated ammonium chloride solution and extracted with ethyl acetate. The organic layer was dried over anhydrous sodium sulfate, filtered and evaporated under reduced pressure to afford [(1R)-2-[2-[(1S)-1-[5-(5-hydroxy-1-tetrahydropyran-2-yl-indazol-3-yl)-3-pyridyl]ethoxy] ethoxy]-1-methyl-ethyl]methanesulfonate as a yellow oil which was used in the next step without further purification.

LCMS method F: [M+H]⁺=520.3, t_{R}=2.08 min

### Preparation of Intermediate 98: (7S,13S)-7,13-dimethyl-19-(oxan-2-yl)-8,11,14-trioxa-4,19,20-triazatetracyclo[13.5.2.1^{2,6}.0^{18,21}]tricosa-1(20),2(23),3, 5,15(22),16, 18(21)-heptaene

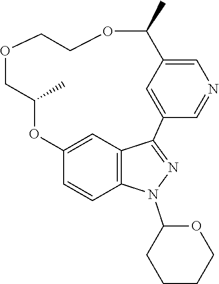 [SMILES: C[C@@H]1OCCOC[C@H](C)Oc2ccc3c(c2)c(nn3C2CCCCO2)-c2cncc1c2]

To a suspension of cesium carbonate (2.928 g, 8.99 mmol) in anhydrous acetonitrile (150 mL) at 50° C. was added dropwise [(1R)-2-[2-[(1S)-1-[5-(5-hydroxy-1-tetrahydropyran-2-yl-indazol-3-yl)-3-pyridyl]ethoxy]ethoxy]-1-methyl-ethyl]methanesulfonate (467 mg, 0.90 mmol) in acetonitrile (200 mL). The reaction mixture was stirred at 50° C. for 30 min. The mixture was cooled to RT, filtered and the filtrate was evaporated under reduced pressure. The residue was purified by silica gel column chromatography using cyclohexane/ethyl acetate 100/0 to 50/50 as eluent to afford (7S,13S)-7,13-dimethyl-19-(oxan-2-yl)-8,11,14-trioxa-4,19,20-triazatetracyclo[13.5.2.1^{2,6}.0^{18,21}]tricosa-1(20),2(23),3,5,15(22),16,18(21)-heptaene as a yellow oil.

LCMS method F: [M+H]⁺=424.3, t_{R}=2.50 min

### Preparation of Example 9: (7S,13S)-7,13-dimethyl-8,11,14-trioxa-4,19,20-triazatetra cyclo[13.5.2.1^{2,6}.0^{18,21}]tricosa-1(20),2(23),3,5,15(22),16,18(21)-heptaene

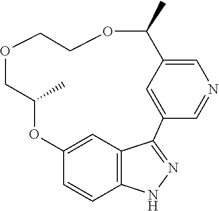 [SMILES: C[C@@H]1OCCOC[C@H](C)Oc2ccc3[nH]nc(c3c2)-c2cncc1c2]

To a solution of (7S,13S)-7,13-dimethyl-19-(oxan-2-yl)-8,11,14-trioxa-4,19,20-triazatetra cyclo[13.5.2.1^{2,6}.0^{18,21}]tricosa-1(20),2(23),3,5,15(22),16,18(21)-heptaene (86 mg, 0.20 mmol) in methanol (2 mL) and water (0.3 mL) was added p-toluenesulfonic acid monohydrate (193 mg, 1.02 mmol). The reaction mixture was stirred at 80° C. for 5 h. The solvent was evaporated under reduced pressure and the residue was partitioned between ethyl acetate and saturated aqueous NaHCO₃ solution. The aqueous layer was extracted with ethyl acetate and the organic layer was washed with brine, dried over anhydrous sodium sulfate, filtered and evaporated under reduced pressure. The residue was purified by silica gel column chromatography using cyclohexane/ethyl acetate 100/0 to 30/70 as eluent to afford (7S,13S)-7,13-dimethyl-8,11,14-trioxa-4,19,20-triazatetracyclo[13.5.2.1^{2,6}.0^{18,21}]tricosa-1(20),2(23),3,5,15(22),16,18(21)-heptaene as a powder.

LCMS method F: [M+H]⁺=340.2, t_{R}=1.87 min

LCMS method G: [M+H]⁺=340.2, t_{R}=2.35 min

¹H NMR (400 MHz, d6-DMSO) δ 13.21-13.20 (1H, m), 8.95 (1H, d, J=2.3 Hz), 8.50-8.44 (2H, m), 8.35 (1H, d, J=2.1 Hz), 7.48-7.45 (1H, m), 7.05-7.02 (1H, m), 4.73-4.68 (1H, m), 4.27-4.22 (1H, m), 3.87-3.75 (3H, m), 3.65-3.49 (3H, m), 1.41-1.31 (6H, m) ppm.

### Example 10: (7R,13S)-7,13-dimethyl-8,11,14-trioxa-4,19,20-triazatetracyclo[13.5.2.1^{2,6}.0^{18,21}]tricosa-1(20),2,4,6(23),15(22),16,18(21)-heptaene

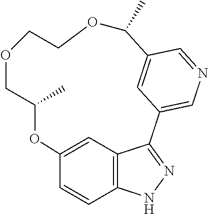 [SMILES: C[C@H]1COCCO[C@H](C)c2cncc(c2)-c2n[nH]c3ccc(cc23)O1]

Example 10 is prepared according to the synthesis route described in general Scheme D, following the same synthesis procedures as for Example 9.

To a solution of (7R,13S)-7,13-dimethyl-19-(oxan-2-yl)-8,11,14-trioxa-4,19,20-triazatetra cyclo[13.5.2.1^{2,6}.0^{18,21}]tricosa-1(20),2,4,6(23),15(22),16,18(21)-heptaene (219 mg, 0.52 mmol) in methanol (5.2 mL) and water (0.8 mL) was added p-toluenesulfonic acid monohydrate (492 mg, 2.59 mmol). The reaction mixture was stirred at 80° C. for 5 h. The solvent was evaporated under reduced pressure and the residue was partitioned between ethyl acetate and saturated aqueous NaHCO₃ solution. The aqueous layer was extracted with ethyl acetate and the combined organic layer was washed with brine, dried over anhydrous sodium sulfate, filtered and evaporated under reduced pressure. The residue was purified by silica gel column chromatography using cycloxane/ethyl acetate 100/0 to 30/70 as eluent to afford (7R,13S)-7,13-dimethyl-8,11,14-trioxa-4,19,20-triazatetracyclo[13.5.2.1^{2,6}.0^{18,21}]tricosa-1(20),2,4,6(23),15(22),16,18(21)-heptaene as a white powder.

LCMS method F: [M+H]⁺=340.2, t_{R}=1.84 min

LCMS method G: [M+H]⁺=340.2, t_{R}=2.30 min

¹H NMR (400 MHz, d6-DMSO) δ 13.24-13.22 (1H, m), 9.06 (1H, d, J=2.3 Hz), 8.67 (1H, d, J=1.9 Hz), 8.54-8.46 (2H, m), 7.47-7.44 (1H, m), 7.07-7.03 (1H, m), 4.71 (1H, q, J=6.5 Hz), 4.39-4.35 (1H, m), 3.83-3.57 (6H, m), 1.45 (3H, d, J=6.6 Hz), 1.34 (3H, d, J=6.8 Hz) ppm.

### Example 11: (8S,13R)-8,13-dimethyl-7,11,14-trioxa-4,19,20-triazatetracyclo[13.5.2.1^{2,6}.0^{18,21}]tricosa-1(20),2(23),3,5,15(22),16,18(21)-heptaene

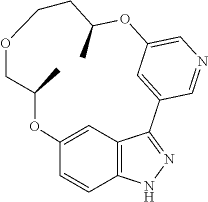 [SMILES: C[C@@H]1COCC[C@H](C)Oc2cncc(c2)-c2n[nH]c3ccc(cc23)O1]

Example 11 is prepared according to the synthesis route described in general Scheme D.

### Preparation of Intermediate 99: (2R)-4-[(tert-butyldiphenylsilyl)oxy]butan-2-ol

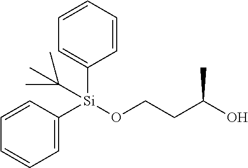 [SMILES: C[C@@H](O)CCO[Si](c1ccccc1)(c1ccccc1)C(C)(C)C]

To a solution of (3R)-butane-1,3-diol (5 g, 55.48 mmol) in THF (277 mL) was added imidazole (7.554 g, 110.96 mmol) and tert-butyldiphenylchlorosilane (14.427 mL, 55.48 mmol). The reaction mixture was stirred at RT for 4 h. Water and ethyl acetate were added and the layers were separated. The aqueous layer was extracted with ethyl acetate and the combined organic layers were dried over anhydrous sodium sulfate, filtered and evaporated under reduced pressure. The residue was purified by silica gel column chromatography using cyclohexan/ethyl acetate 100/0 to 90/10 as eluent to afford (2R)-4-[(tert-butyldiphenylsilyl)oxy]butan-2-ol as a colorless oil.

LCMS method F: [M+H]⁺=329.3, t_{R}=3.39 min

### Preparation of Intermediate 100: 3-bromo-5-{[(2S)-4-[(tert-butyldiphenylsilyl)oxy] butan-2-yl]oxy}pyridine

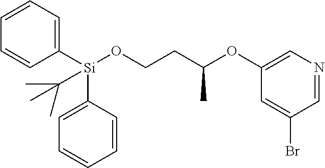 [SMILES: C[C@@H](CCO[Si](c1ccccc1)(c1ccccc1)C(C)(C)C)Oc1cncc(Br)c1]

To a solution of 5-bromopyridin-3-ol (1 g, 5.75 mmol), (2R)-4-[(tert-butyldiphenylsilyl)oxy] butan-2-ol (2.454 g, 7.47 mmol) and triphenylphosphine (2.261 g, 8.62 mmol) in dry THF (30 mL) under argon was added DIAD (1.697 mL, 8.62 mmol). The reaction mixture was stirred at RT overnight. The solvent was evaporated under reduced pressure. The residue was purified by silica gel column chromatography using cyclohexane/ethyl acetate 95/5 as eluent to afford 3-bromo-5-{[(2S)-4-[(tert-butyldiphenylsilyl)oxy]butan-2-yl]oxy}pyridine as a pink oil.

LCMS method F: [M+H]⁺=486.1, t_{R}=3.91 min

### Preparation of Intermediate 101: (3S)-3-[(5-bromopyridin-3-yl)oxy]butan-1-ol

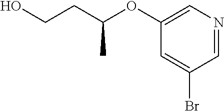

To a solution of 3-bromo-5-{[(2S)-4-[(tert-butyldiphenylsilyl)oxy]butan-2-yl]oxy}pyridine (2.815 g, 5.81 mmol) in THF (30 mL) was added dropwise at RT TBAF (1M solution in THF) (6.39 mL, 6.39 mmol). The reaction mixture was stirred at RT for 16 h. The reaction mixture was poured into ice water and stirred for 20 min. The aqueous phase was extracted with ethyl acetate and the combined organic layer were washed with brine, dried over anhydrous sodium sulfate and evaporated under reduced pressure. The residue was purified by silica gel column chromatography using cyclohexane/ethyl acetate 90/10 as eluent to afford (3S)-3-[(5-bromopyridin-3-yl)oxy]butan-1-ol as a white solid.

LCMS method F: [M+H]⁺=246.1, t_{R}=1.91 min

### Preparation of Intermediate 102: (2S)-4-trityloxybutan-2-ol

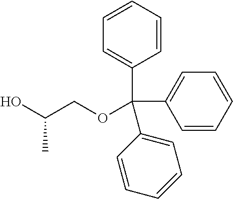 [SMILES: C[C@H](O)COC(c1ccccc1)(c1ccccc1)c1ccccc1]

To solution of (2S)-propane-1,2-diol (962 μL, 13.14 mmol) in dichloromethane (20 mL) at 0° C. was added triethylamine (2.381 mL, 17.09 mmol) followed with a dropwise addition of trityl chloride (3.664 g, 13.14 mmol) in dichloromethane (10 mL). The reaction mixture was stirred at RT overnight. Water was added to the suspension and the phases were separated. The aqueous phase was extracted with dichloromethane and the combined organic layers were washed with brine, dried over anhydrous sodium sulfate, filtered and the solvent was removed under reduced pressure to afford (2S)-4-trityloxybutan-2-ol was obtained as a colorless oil.

LCMS method F: [M+Na]⁺=341.2, t_{R}=3.03 min

### Preparation of Intermediate 103: {[(2S)-2-(benzyloxy)propoxy]diphenylmethyl}benzene

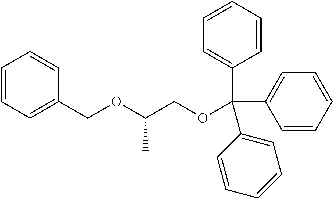 [SMILES: C[C@@H](COC(c1ccccc1)(c1ccccc1)c1ccccc1)OCc1ccccc1]

To a solution of [[(2S)-2-benzyloxypropoxy]-diphenyl-methyl]benzene (4.407 g, 13.84 mmol) in DMF (30 mL) at 0° C. was added sodium hydride (60% dispersion in mineral oil) (830 mg, 20.76 mmol). The reaction mixture was stirred for 20 min. A solution of benzyl bromide (3.292 mL, 27.68 mmol) in DMF (5 mL) was added dropwise and the suspension was stirred at RT for 5 h. Water was added and the aqueous phase was extracted with ethyl acetate. The combined organic layers were washed with brine, dried over anhydrous sodium sulfate, filtered and the solvent was evaporated under reduced pressure. The residue was purified by silica gel column chromatography using cyclohexane/ethyl acetate 90/10 as eluent to afford {[(2S)-2-(benzyloxy)propoxy]diphenylmethyl}benzene as a colorless oil.

LCMS method F: [M+H]⁺=431.2, t_{R}=3.69 min

### Preparation of Intermediate 104: (2S)-2-benzyloxypropan-1-ol

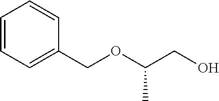

A solution of {[(2S)-2-(benzyloxy)propoxy]diphenylmethyl}benzene (4.324 g, 10.58 mmol) in a mixture of methanol, acetic acid and water (53 mL) was stirred at 90° C. overnight. The solvent was evaporated under reduced pressure and the residue was dissolved in ethyl acetate. The organic phase was washed with a saturated aqueous NaHCO₃ solution and brine, dried over anhydrous sodium sulfate, filtered and evaporated under reduced pressure. The residue was purified by silica gel column chromatography using cyclohexane/ethyl acetate 90/10 to 70/30 as eluent to afford (2S)-2-benzyloxypropan-1-ol as a colorless oil.

LCMS method F: [M+H]⁺=167.1, t_{R}=1.91 min

### Preparation of Intermediate 105: (2S)-2-(benzyloxy)propyl 4-methylbenzene-1-sulfonate

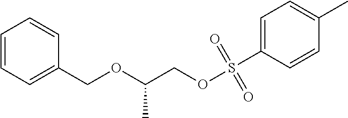 [SMILES: Cc1ccc(S(=O)(=O)OC[C@H](C)OCc2ccccc2)cc1]

To a suspension of (2S)-2-(benzyloxy)propan-1-ol (900 mg, 5.41 mmol) in pyridine (6 mL) at 0° C. was added p-toluenesulfonyl chloride (1.239 g, 6.5 mmol). The reaction mixture was stirred at RT overnight. The mixture was diluted with ethyl acetate, washed with water and brine. The organic layer was dried over anhydrous sodium sulfate, filtered and evaporated under reduced pressure to afford (2S)-2-(benzyloxy)propyl 4-methylbenzene-1-sulfonate as a yellow oil.

LCMS method F: [M+H]⁺=321.1, t_{R}=2.95 min

### Preparation of Intermediate 106: 3-{[(2S)-4-[(2S)-2-(benzyloxy)propoxy]butan-2-yl]oxy}-5-bromopyridine

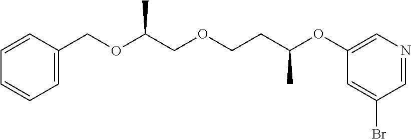 [SMILES: C[C@@H](COCC[C@H](C)Oc1cncc(Br)c1)OCc1ccccc1]

To a solution of (3S)-3-[(5-bromopyridin-3-yl)oxy]butan-1-ol (425 mg, 1.73 mmol) in DMF (6 mL) at 0° C. was added sodium hydride (60% dispersion in mineral oil) (104 mg, 2.59 mmol) and (2S)-2-(benzyloxy)propyl 4-methylbenzene-1-sulfonate (1.66 g, 5.18 mmol) in DMF (2 mL). The reaction mixture was stirred at RT for 4 h. The mixture was diluted with ethyl acetate, washed with water and brine. The organic layer was dried over anhydrous sodium sulfate, filtered and evaporated under reduced pressure. The residue was purified by silica gel column chromatography using cyclohexane/ethyl acetate 100/0 to 85/15 as eluent to afford 3-{[(2S)-4-[(2S)-2-(benzyloxy)propoxy]butan-2-yl]oxy}-5-bromopyridine as a clear oil.

LCMS method F: [M+H]⁺=394.1-396.1, t_{R}=3.18 min

### Preparation of Intermediate 107: [3-[5-[(1S)-3-[(2S)-2-benzyloxypropoxy]-1-methyl-propoxy]-3-pyridyl]-1-tetrahydropyran-2-yl-indazol-5-yl]oxy-tert-butyl-dimethyl-silane

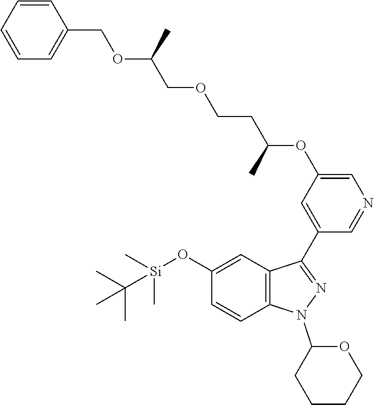 [SMILES: C[C@@H](COCC[C@H](C)Oc1cncc(-c2nn(C3CCCCO3)c3ccc(O[Si](C)(C)C(C)(C)C)cc23)c1)OCc1ccccc1]

To a suspension of 3-{[(2S)-4-[(2S)-2-(benzyloxy)propoxy]butan-2-yl]oxy}-5-bromopyridine (530 mg, 1.34 mmol) in dioxane (6 mL) and water (0.7 mL) was added 5-[(tert-butyldimethylsilyl)oxy]-1-(oxan-2-yl)-3-(4,4,5,5-tetramethyl-1,3,2-dioxaborolan-2-yl)-1H-indazole (801 mg, 1.75 mmol) and potassium phosphate tribasic (856 mg, 4.03 mmol). The reaction mixture was degassed by bubbling under argon for 15 min and tetrakis(triphenylphosphine)palladium(0) (78 mg, 0.07 mmol) and Xphos (64 mg, 0.13 mmol) were added. The reaction was heated at 90° C. under microwave irradiations for 1 h. The reaction mixture was poured into water and extracted with ethyl acetate. The organic layer was washed with brine, dried over anhydrous sodium sulfate and evaporated under reduced pressure. The residue was purified by silica gel column chromatography using cyclohexane/ethyl acetate 80/20 as eluent to afford [3-[5-[(1S)-3-[(2S)-2-benzyloxypropoxy]-1-methyl-propoxy]-3-pyridyl]-1-tetrahydropyran-2-yl-indazol-5-yl]oxy-tert-butyl-dimethyl-silane as a light yellow oil.

LCMS method F: [M+H]⁺=646.4, t_{R}=3.96 min

### Preparation of Intermediate 108: (2S)-1-[(3S)-3-[[5-[5-[tert-butyl(dimethyl)silyl]oxy-1-tetrahydropyran-2-yl-indazol-3-yl]-3-pyridyl]oxy]butoxy]propan-2-ol

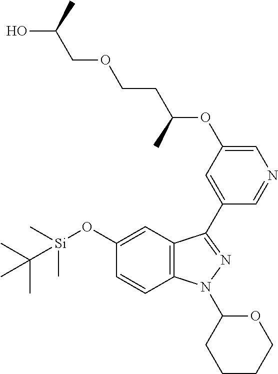 [SMILES: C[C@H](O)COCC[C@H](C)Oc1cncc(-c2nn(C3CCCCO3)c3ccc(O[Si](C)(C)C(C)(C)C)cc23)c1]

To a solution of [3-[5-[(1S)-3-[(2S)-2-benzyloxypropoxy]-1-methyl-propoxy]-3-pyridyl]-1-tetrahydropyran-2-yl-indazol-5-yl]oxy-tert-butyl-dimethyl-silane (800 mg, 1.24 mmol) in methanol (5 mL) under argon was added palladium hydroxyde (80 mg, 0.75 mmol). The reaction mixture was stirred under hydrogen atmosphere at 50° C. for 16 h. Palladium hydroxyde was filtered and the solvent was evaporated under reduced pressure to afford (2S)-1-[(3S)-3-[[5-[5-[tert-butyl(dimethyl)silyl]oxy-1-tetrahydropyran-2-yl-indazol-3-yl]-3-pyridyl]oxy]butoxy] propan-2-ol as a light yellow oil.

LCMS method F: [M+H]⁺=556.4, t_{R}=3.45 min

### Preparation of Intermediate 109: [(1S)-2-[(3S)-3-[[5-[5-[tert-butyl(dimethyl)silyl] oxy-1-tetrahydropyran-2-yl-indazol-3-yl]-3-pyridyl]oxy]butoxy]-1-methyl-ethyl] methanesulfonate

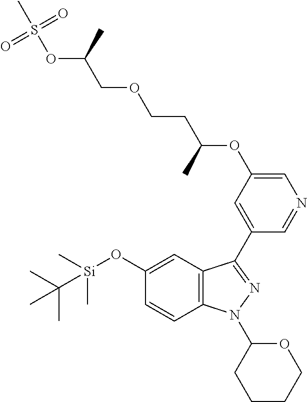 [SMILES: C[C@@H](CCOC[C@H](C)OS(C)(=O)=O)Oc1cncc(-c2nn(C3CCCCO3)c3ccc(O[Si](C)(C)C(C)(C)C)cc23)c1]

To a solution of (2S)-1-[(3S)-3-[[5-[5-[tert-butyl(dimethyl)silyl]oxy-1-tetrahydropyran-2-yl-indazol-3-yl]-3-pyridyl]oxy]butoxy]propan-2-ol (637 mg, 1.15 mmol) in dichloromethane (6 mL) was added triethylamine (319 μL, 2.29 mmol) and dropwise methanesulfonyl chloride (44 μL, 0.57 mmol). The reaction mixture was stirred at RT for 2 h. The reaction mixture was poured into water and the organic layer was washed with brine, dried over anhydrous sodium sulfate and evaporated under reduced pressure to afford [(1S)-2-[(3S)-3-[[5-[5-[tert-butyl(dimethyl)silyl]oxy-1-tetrahydropyran-2-yl-indazol-3-yl]-3-pyridyl]oxy]butoxy]-1-methyl-ethyl] methanesulfonate as a yellow oil.

LCMS method F: [M+H]⁺=634.3, t_{R}=3.56 min

### Preparation of Intermediate 110: [(1S)-2-[(3S)-3-[[5-(5-hydroxy-1-tetrahydropyran-2-yl-indazol-3-yl)-3-pyridyl]oxy]butoxy]-1-methyl-ethyl]methanesulfonate

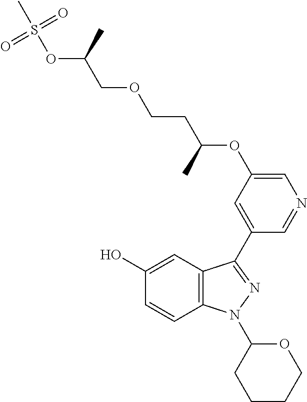 [SMILES: C[C@@H](CCOC[C@H](C)OS(C)(=O)=O)Oc1cncc(-c2nn(C3CCCCO3)c3ccc(O)cc23)c1]

To a solution of [(1S)-2-[(3S)-3-[[5-[5-[tert-butyl(dimethyl)silyl]oxy-1-tetrahydropyran-2-yl-indazol-3-yl]-3-pyridyl]oxy]butoxy]-1-methyl-ethyl] methanesulfonate (727 mg, 1.15 mmol) in THF (3 mL) at RT was added TBAF (1M solution in THF) (1.72 mL, 1.72 mmol). The reaction mixture was stirred at RT for 1 h. The reaction mixture was diluted with a saturated aqueous ammonium chloride solution and extracted with ethyl acetate. The combined organic layers were dried over anhydrous sodium sulfate and evaporated under reduced pressure to afford [(1S)-2-[(3S)-3-[[5-(5-hydroxy-1-tetrahydropyran-2-yl-indazol-3-yl)-3-pyridyl]oxy] butoxy]-1-methyl-ethyl] methanesulfonate as a yellow oil which was used in the next step without further purification.

LCMS method F: [M+H]⁺=520.3, t_{R}=2.40 min

### Preparation of Intermediate 111: (8S,13R)-8,13-dimethyl-19-(oxan-2-yl)-7,11,14-trioxa-4,19,20-triazatetracyclo[13.5.2.1^{2,6}.0^{18,21}]tricosa-1(20),2(23),3,5,15(22),16, 18(21)-heptaene

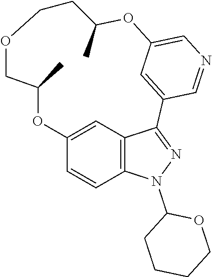 [SMILES: C[C@@H]1COCC[C@H](C)Oc2cncc(c2)-c2nn(C3CCCCO3)c3ccc(cc23)O1]

To a suspension of cesium carbonate (1.317 g, 4.04 mmol) in anhydrous DMF (70 mL) at 80° C. was added dropwise [(1S)-2-[(3S)-3-[[5-(5-hydroxy-1-tetrahydropyran-2-yl-indazol-3-yl)-3-pyridyl]oxy]butoxy]-1-methyl-ethyl] methanesulfonate (700 mg, 1.35 mmol) in DMF (60 mL). The reaction mixture was stirred at 80° C. for 30 min. The reaction mixture was filtered and the solvent was removed under reduced pressure. The residue was purified by silica gel column chromatography using cyclohexane/ethyl acetate 50/50 as eluent to afford (8S,13R)-8,13-dimethyl-19-(oxan-2-yl)-7,11,14-trioxa-4,19,20-triazatetracyclo[13.5.2.1^{2,6}.0^{18,21}]tricosa-1(20),2(23),3,5,15(22),16,18(21)-heptaene as a white foam.

LCMS method F: [M+H]⁺=424.2, t_{R}=2.93 min

### Preparation of Example 11: (8S,13R)-8,13-dimethyl-7,11,14-trioxa-4,19,20-triazatetracyclo[13.5.2.1^{2,6}.0^{18,21}]tricosa-1(20),2(23),3, 5,15(22),16,18(21)-heptaene

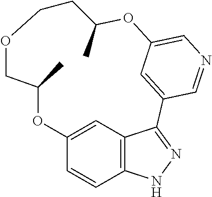 [SMILES: C[C@@H]1COCC[C@H](C)Oc2cncc(c2)-c2n[nH]c3ccc(cc23)O1]

To a solution of ((8S,13R)-8,13-dimethyl-19-(oxan-2-yl)-7,11,14-trioxa-4,19,20-triazatetracyclo[13.5.2.1^{2,6}.0^{18,21}]tricosa-1(20),2(23),3,5,15(22),16,18(21)-heptaene (314 mg, 0.74 mmol) in methanol (2.5 mL) and water (0.4 mL) was added p-toluenesulfonic acid monohydrate (705 mg, 3.71 mmol). The reaction mixture was stirred at 80° C. for 16 h. The reaction mixture was dissolved in ethyl acetate and a saturated aqueous NaHCO₃ solution. After separation, the aqueous layer was extracted with ethyl acetate. The combined organic layers were washed with brine, dried over sodium sulfate, filtered and evaporated under reduced pressure. The resulting solid was recrystallized from acetonitrile, filtered and dried to afford (8S,13R)-8,13-dimethyl-7,11,14-trioxa-4,19,20-triazatetracyclo[13.5.2.1^{2,6}.0^{18,21}]tricosa-1(20),2(23),3,5,15(22),16,18(21)-heptaene as a solid.

LCMS method F: [M+H]⁺=340.3, t_{R}=2.14 min

LCMS method G: [M+H]⁺=340.2, t_{R}=2.62 min

¹H NMR (400 MHz, d6-DMSO) δ 13.25 (1H, s), 8.84 (1H, d, J=1.3 Hz), 8.49 (1H, d, J=2.1 Hz), 8.18-8.16 (2H, m), 7.49-7.46 (1H, m), 7.04 (1H, dd, J=2.3, 8.9 Hz), 4.92-4.85 (1H, m), 4.57-4.50 (1H, m), 3.76 (1H, dd, J=7.0, 10.8 Hz), 3.69-3.53 (3H, m), 2.38-2.30 (1H, m), 1.67-1.60 (1H, m), 1.49-1.46 (3H, m), 1.35 (3H, d, J=6.6 Hz) ppm.

### Example 12: (8S,13S)-8,13-dimethyl-7,11,14-trioxa-4,19,20-triazatetracyclo[13.5.2.1^{2,6}.0^{18,21}]tricosa-1(20),2(23),3,5,15(22),16,18(21)-heptaene

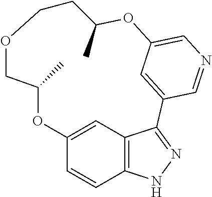 [SMILES: C[C@H]1CCOC[C@H](C)Oc2ccc3[nH]nc(c3c2)-c2cncc(c2)O1]

Example 12 is prepared according to the synthesis route described in general Scheme D, following the same synthesis procedures as for Example 11.

To a solution of ((8S,13S)-8,13-dimethyl-19-(oxan-2-yl)-7,11,14-trioxa-4,19,20-triazatetra cyclo[13.5.2.1^{2,6}.0^{18,21}]tricosa-1(20),2(23),3,5,15(22),16,18(21)-heptaene (61 mg, 0.14 mmol) in methanol (0.9 mL) and water (0.15 mL) was added p-toluenesulfonic acid monohydrate (137 mg, 0.72 mmol). The reaction mixture was stirred at 80° C. for 16 h. The reaction mixture was dissolved in ethyl acetate and a saturated aqueous NaHCO₃ solution. After separation, the aqueous layer was extracted with ethyl acetate. The combined organic layers were washed with brine, dried over sodium sulfate, filtered and evaporated under reduced pressure. The resulting solid was recrystallized from acetonitrile, filtered and dried to afford (8S,13S)-8,13-dimethyl-7,11,14-trioxa-4,19,20-triazatetracyclo[13.5.2.1^{2,6}.0^{18,21}]tricosa-1(20),2(23),3,5,15(22),16, 18(21)-heptaene as a solid.

LCMS method F: [M+H]⁺=340.3, t_{R}=2.12 min

LCMS method G: [M+H]⁺=340.2, t_{R}=2.56 min Chiral analysis shows e.e. 98.5%

¹H NMR (400 MHz, d6-DMSO) δ 13.24 (1H, s), 8.70 (1H, d, J=1.5 Hz), 8.25-8.18 (2H, m), 8.00 (1H, t, J=2.1 Hz), 7.51-7.48 (1H, m), 7.03 (1H, dd, J=2.3, 8.9 Hz), 4.83-4.75 (1H, m), 4.52-4.46 (1H, m), 3.87 (1H, dd, J=4.6, 12.7 Hz), 3.76-3.71 (1H, m), 3.58 (2H, t, J=13.0 Hz), 2.41-2.32 (1H, m), 1.73-1.64 (1H, m), 1.40 (6H, dd, J=6.5, 19.4 Hz) ppm.

### Example 13: (7R,13R)-7,13-dimethyl-8,11,14-trioxa-4,19,20-triazatetracyclo[13.5.2.1^{2,6}. 0^{18,21}]tricosa-1(20),2(23),3,5,15(22),16,18(21)-heptaene hydrochloride

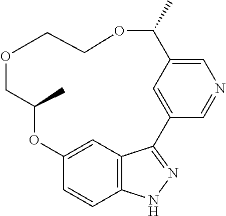 [SMILES: C[C@@H]1COCCO[C@H](C)c2cncc(c2)-c2n[nH]c3ccc(cc23)O1]

Example 13 is prepared according to the synthesis route described in general Scheme D, following the same synthesis procedures as for Example 9.

To a solution of (7R,13R)-7,13-dimethyl-19-(oxan-2-yl)-8,11,14-trioxa-4,19,20-triazatetra cyclo[13.5.2.1^{2,6}.0^{18,21}]tricosa-1(20),2(23),3,5,15(22),16,18(21)-heptaene (371 mg, 0.88 mmol) in methanol (8.8 mL) and water (1.3 mL) was added p-toluenesulfonic acid monohydrate (833 mg, 4.38 mmol). The reaction mixture was heated to 80° C. for 5 h. The solvent was evaporated under reduced pressure and the residue was partitioned between ethyl acetate and a saturated aqueous NaHCO₃ solution. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 30/70 as eluent. The resulting solid was dissolved in ethanol (2.2 mL) and 1M aqueous hydrochloric acid solution (454 μL, 0.45 mmol) was added. The solution was stirred at RT for 10 min. The solvent was evaporated under reduced pressure. The resulting solid was recrystallized from ethanol to afford (7R,13R)-7,13-dimethyl-8,11,14-trioxa-4,19,20-triazatetracyclo[13.5.2.1^{2,6}.0^{18,21}]tricosa-1(20),2(23),3, 5, 15(22),16,18(21)-heptaene hydrochloride as a solid.

LCMS method F: [M+H]⁺=340.3, t_{R}=1.78 min

LCMS method G: [M+H]⁺=340.3, t_{R}=2.17 min

¹H NMR (400 MHz, d6-DMSO) δ 13.56-13.52 (1H, m), 9.15 (1H, d, J=1.9 Hz), 8.90-8.87 (1H, m), 8.75 (1H, d, J=1.1 Hz), 8.35 (1H, d, J=2.1 Hz), 7.55-7.52 (1H, m), 7.08 (1H, dd, J=2.1, 8.9 Hz), 4.86-4.80 (1H, m), 4.29-4.24 (1H, m), 3.88-3.79 (3H, m), 3.75-3.60 (2H, m), 3.60-3.50 (1H, m), 1.47-1.43 (3H, m), 1.35 (3H, d, J=6.6 Hz) ppm.

### Example 14: (7S,13R)-7,13-dimethyl-8,11,14-trioxa-4,19,20-triazatetracyclo[13.5.2.1^{2,6}. 0^{18,21}]tricosa-1(20),2(23),3,5,15(22),16,18(21)-heptaene hydrochloride

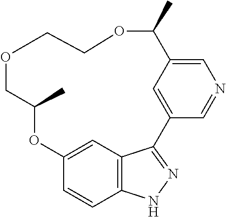 [SMILES: C[C@@H]1COCCO[C@@H](C)c2cncc(c2)-c2n[nH]c3ccc(cc23)O1]

Example 14 is prepared according to the synthesis route described in general Scheme D, following the same synthesis procedures as for Example 9.

To a solution of (7S,13R)-7,13-dimethyl-19-(oxan-2-yl)-8,11,14-trioxa-4,19,20-triazatetra cyclo[13.5.2.1^{2,6}.0^{18,21}]tricosa-1(20),2(23),3,5,15(22),16,18(21)-heptaene (367 mg, 0.87 mmol) in methanol (8.7 mL) and water (1.2 mL) was added p-toluenesulfonic acid monohydrate (824 mg, 4.33 mmol). The reaction mixture was heated to 80° C. for 5 h. The solvent was evaporated under reduced pressure and the residue was partitioned between ethyl acetate and a saturated aqueous NaHCO₃ solution. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 30/70 as eluent. The resulting solid was dissolved in ethanol (3 mL) and 1M aqueous hydrochloric acid solution (603 μL, 0.60 mmol) was added. The solution was stirred at RT for 2 h. The solvent was evaporated under reduced pressure. The resulting product was recrystallized from acetonitrile to afford (7S,13R)-7,13-dimethyl-8,11,14-trioxa-4,19,20-triazatetracyclo[13.5.2.1^{2,6}.0^{18,21}]tricosa-1(20),2(23),3, 5,15(22),16,18(21)-heptaene hydrochloride as a powder.

LCMS method F: [M+H]⁺=340.3, t_{R}=2.32 min

LCMS method J: [M+H]⁺=340.3, t_{R}=2.15 min

¹H NMR (400 MHz, d6-DMSO) δ 13.58-13.52 (1H, m), 9.22-9.21 (1H, m), 8.92-8.90 (1H, m), 8.72-8.66 (2H, m), 7.53-7.50 (1H, m), 7.09 (1H, dd, J=2.3, 8.9 Hz), 4.82 (1H, q, J=6.5 Hz), 4.46-4.39 (1H, m), 3.67 (6H, d, J=16.3 Hz), 1.50 (3H, d, J=6.6 Hz), 1.35 (3H, d, J=6.6 Hz) ppm.

### Example 15: (6S)-6-methyl-9,13-dioxa-4,5,18,19-tetraazatetracyclo[12.5.2.1^{2,5}.0^{17,20}] docosa-1(19),2(22),3,14(21),15,17(20)-hexaene

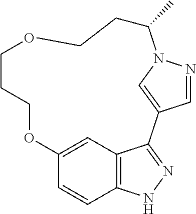 [SMILES: C[C@H]1CCOCCCOc2ccc3[nH]nc(c3c2)-c2cnn1c2]

Example 15 is prepared according to the synthesis route described in general Scheme B, following the same synthesis procedures as for Example 8. The TBDMS removal and macrocyclization were performed in one step.

### Preparation of Intermediate 112: (6S)-6-methyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene

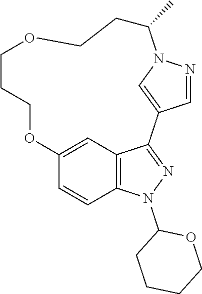 [SMILES: C[C@H]1CCOCCCOc2ccc3c(c2)c(nn3C2CCCCO2)-c2cnn1c2]

To a suspension of cesium carbonate (97 mg, 0.30 mmol) in DMF (17.5 mL) at 90° C. was added dropwise 3-[(3S)-3-[4-[5-[tert-butyl(dimethyl)silyl]oxy-1-tetrahydropyran-2-yl-indazol-3-yl] pyrazol-1-yl]butoxy]propylmethanesulfonate (45 mg, 0.07 mmol) in DMF (17.5 mL). After addition the resulting reaction mixture was stirred at 90° C. for 1 h. The reaction mixture was concentrated under reduced pressure, diluted with brine and extracted with ethyl acetate. The combined organic layers were dried over anhydrous sodium sulfate and evaporated under reduced pressure. The residue was purified by silica gel column chromatography dichloromethane/ethyl acetate 100/00 to 70/30 as eluent afford (6S)-6-methyl-18-(oxan-2-yl)-9,13-dioxa-4,5,18,19-tetraazatetracyclo[12.5.2.1^{2,5}.0^{17,20}]docosa-1(19),2(22),3,14(21),15, 17(20)-hexaene as a colorless oil.

LCMS method F: [M+H]⁺=397.4, t_{R}=2.62 min

### Preparation of Example 15: (6S)-6-methyl-9,13-dioxa-4,5,18,19-tetraazatetracyclo [12.5.2.1^{2,5}.0^{17,20}]docosa-1(19),2(22),3,14(21),15,17(20)-hexaene

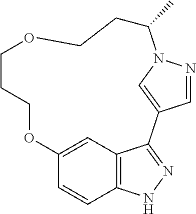 [SMILES: C[C@H]1CCOCCCOc2ccc3[nH]nc(c3c2)-c2cnn1c2]

To a solution of (6S)-6-methyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene (25 mg, 0.06 mmol) in methanol (3.5 mL) and water (0.5 mL) was added p-toluenesulfonic acid monohydrate (60 mg, 0.32 mmol). The reaction mixture was stirred at 65° C. for 2 h. The reaction mixture was concentrated under reduced pressure and the residue was neutralized by slow addition of a saturated aqueous NaHCO₃ solution. The residue was diluted with ethyl acetate. The layers were separated and the aqueous layer was extracted with ethyl acetate. The combined organic layers were washed with brine, dried over anhydrous sodium sulfate, filtered and evaporated under reduced pressure. The solid was recrystallized from acetonitrile to afford (6S)-6-methyl-9,13-dioxa-4,5,18,19-tetraazatetracyclo[12.5.2.1^{2,5}.0^{17,20}]docosa-1(19),2(22),3,14(21),15,17(20)-hexaene as a cream solid.

LCMS method F: [M+H]⁺=313.3, t_{R}=2.04 min

LCMS method G: [M+H]⁺=313.3, t_{R}=2.04 min

¹H NMR (400 MHz, d6-DMSO) δ 12.68 (1H, s), 8.60-8.59 (1H, m), 7.68 (1H, s), 7.47-7.37 (2H, m), 6.94 (1H, dd, J=2.5, 8.9 Hz), 4.60-4.54 (1H, m), 4.36-4.29 (2H, m), 3.60-3.41 (4H, m), 2.25-2.19 (2H, m), 2.08-1.98 (2H, m), 1.53 (3H, d, J=6.8 Hz) ppm.

### Example 16: (13S)-13-methyl-7,10,14-trioxa-5,19,20,23-tetraazatetracyclo[13.5.2.1^{2,6}. 0^{18,21}]tricosa-1(20),2(23),3,5,15(22),16,18(21)-heptaene

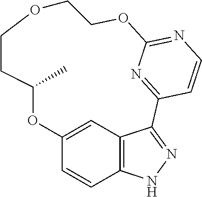 [SMILES: C[C@H]1CCOCCOc2nccc(n2)-c2n[nH]c3ccc(cc23)O1]

Example 16 is prepared according to the synthesis route described in general Scheme C, following the same synthesis procedures as for Example 7. A Mitsunobu reaction is used for the macrocyclization step.

### Preparation of Intermediate 113: (13S)-13-methyl-19-(oxan-2-yl)-7,10,14-trioxa-5, 19,20,23-tetraazatetracyclo[13.5.2.1^{2,6}.0^{18,21}]tricosa-1(20),2(23),3,5,15(22),16,18(21)-heptaene

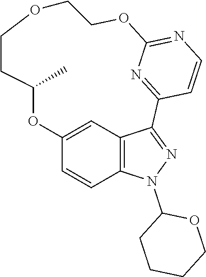 [SMILES: C[C@H]1CCOCCOc2nccc(n2)-c2nn(C3CCCCO3)c3ccc(cc23)O1]

To a solution of 3-[2-[2-[(3R)-3-hydroxybutoxy]ethoxy]pyrimidin-4-yl]-1-tetrahydropyran-2-yl-indazol-5-ol (90 mg, 0.21 mmol) in dry 2-methyltetrahydrofuran (0.40 mL) and toluene (1.70 ml) were added triphenylphosphine (110 mg, 0.42 mmol). The reaction mixture was stirred at 0° C. for 30 min and DIAD (82 μL, 0.42 mmol) was added dropwise. The reaction mixture was stirred at RT for 4 h. The solvent was evaporated under reduced pressure. The residue was purified by silica gel column chromatography using cyclohexane/ethyl acetate 100/0 to 60/40 as eluent to afford (13S)-13-methyl-19-(oxan-2-yl)-7,10,14-trioxa-5,19,20,23-tetraazatetracyclo[13.5.2.1^{2,6}.0^{18,21}]tricosa-1(20),2(23),3,5,15(22),16,18(21)-heptaene as a white powder.

LCMS method F: [M+H]⁺=411.2, t_{R}=3.07 min

### Preparation of Example 16: (13S)-13-methyl-7,10,14-trioxa-5,19,20,23-tetraazatetra cyclo[13.5.2.1^{2,6}.0^{18,21}]tricosa-1(20),2(23),3,5,15(22),16,18(21)-heptaene

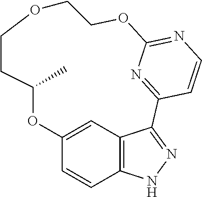 [SMILES: C[C@H]1CCOCCOc2nccc(n2)-c2n[nH]c3ccc(cc23)O1]

To a solution of (13S)-13-methyl-19-(oxan-2-yl)-7,10,14-trioxa-5,19,20,23-tetraazatetra cyclo[13.5.2.1^{2,6}.0^{18,21}]tricosa-1(20),2(23),3,5,15(22),16,18(21)-heptaene (35 mg, 0.085 mmol) in methanol (1.1 mL) and water (0.10 mL) was added p-toluenesulfonic acid monohydrate (81 mg, 0.426 mmol). The reaction mixture was stirred at 70° C. for 48 h. The solvent was evaporated under reduced pressure and the residue was dissolved in ethyl acetate. A saturated aqueous NaHCO₃ solution was added and the aqueous layer was extracted with ethyl acetate. The combined organic layers were washed with brine dried over anhydrous magnesium sulfate, filtered and evaporated under reduced pressure. The residue was purified by silica gel column chromatography using cyclohaxane/ethyl acetate 100/0 to 60/40 as eluent to afford (13S)-13-methyl-7,10,14-trioxa-5,19,20,23-tetraazatetracyclo[13.5.2.1^{2,6}.0^{18,21}]tricosa-1(20),2(23),3,5, 15(22),16,18(21)-heptaene as a powder.

LCMS method F: [M+H]⁺=327.2, t_{R}=2.34 min

LCMS method G: [M+H]⁺=327.2, t_{R}=2.44 min

¹H NMR (400 MHz, d6-DMSO) δ 13.70 (1H, s), 8.60 (1H, d, J=5.1 Hz), 8.16-8.15 (1H, m), 7.76 (1H, d, J=5.1 Hz), 7.57-7.54 (1H, m), 7.03 (1H, dd, J=2.5, 8.9 Hz), 5.01-4.90 (1H, m), 4.75-4.63 (1H, m), 4.40-4.27 (1H, m), 4.23-4.13 (1H, m), 3.71-3.65 (3H, m), 2.42-2.33 (1H, m), 1.40 (3H, d, J=6.1 Hz), 1.24 (1H, s) ppm.

### Example 17: (8R,13R)-8,13-dimethyl-7,11,14-trioxa-4,19,20-triazatetracyclo[13.5.2.1^{2,6}. 0^{18,21}]tricosa-1(20),2(23),3,5,15(22),16,18(21)-heptaene

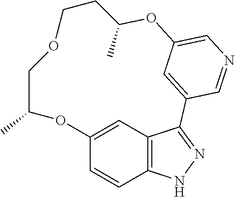 [SMILES: C[C@@H]1CCOC[C@@H](C)Oc2ccc3[nH]nc(c3c2)-c2cncc(c2)O1]

Example 17 is prepared according to the synthesis route described in general Scheme D, following the same synthesis procedures as for Example 11.

To a solution of ((8R,13R)-8,13-dimethyl-19-(oxan-2-yl)-7,11,14-trioxa-4,19,20-triazatetra cyclo[13.5.2.1^{2,6}.0^{18,21}]tricosa-1(20),2(23),3,5,15(22),16,18(21)-heptaene (67 mg, 0.16 mmol) in methanol (0.9 mL) and water (0.15 mL) was added p-toluenesulfonic acid monohydrate (90 mg, 0.47 mmol). The reaction mixture was stirred at 80° C. for 16 h. The reaction mixture was diluted with ethyl acetate and a saturated aqueous NaHCO₃ solution. The aqueous layer was extracted with ethyl acetate. The combined organic layers were washed with brine, dried over anhydrous sodium sulfate, filtered and evaporated under reduced pressure. The resulting solid was recrystallized from acetonitrile, filtered and dried under reduced pressure to afford (8R,13R)-8,13-dimethyl-7,11,14-trioxa-4,19,20-triazatetracyclo[13.5.2.1^{2,6}.0^{18,21}]tricosa-1(20),2(23),3,5,15(22),16,18(21)-heptaene as a solid.

LCMS method F: [M+H]⁺=340.3, t_{R}=2.13 min

LCMS method G: [M+H]⁺=340.3, t_{R}=2.46 min

¹H NMR (400 MHz, d6-DMSO) 13.25-13.25 (1H, m), 8.70 (1H, d, J=1.1 Hz), 8.25-8.18 (2H, m), 8.00 (1H, s), 7.52-7.48 (1H, m), 7.03 (1H, dd, J=2.3, 8.9 Hz), 4.82-4.75 (1H, m), 4.52-4.46 (1H, m), 3.87 (1H, dd, J=4.6, 12.7 Hz), 3.76-3.71 (1H, m), 3.64-3.55 (2H, m), 2.41-2.34 (1H, m), 1.68 (1H, dd, J=11.1, 13.6 Hz), 1.40 (6H, dd, J=6.3, 19.4 Hz) ppm.

### Example 18: (8R,13S)-8,13-dimethyl-7,11,14-trioxa-4,19,20-triazatetracyclo[13.5.2.1^{2,6}. 0^{18,21}]tricosa-1(20),2(23),3,5,15(22),16,18(21)-heptaene

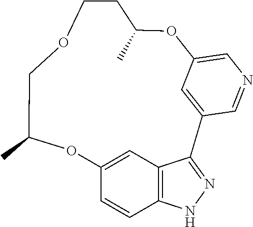 [SMILES: C[C@@H]1CCOC[C@H](C)Oc2ccc3[nH]nc(c3c2)-c2cncc(c2)O1]

Example 18 is prepared according to the synthesis route described in general Scheme D, following the same synthesis procedures as for Example 11.

To a solution of ((8R,13S)-8,13-dimethyl-19-(oxan-2-yl)-7,11,14-trioxa-4,19,20-triaza tetracyclo[13.5.2.1^{2,6}.0^{18,21}]tricosa-1(20),2(23),3,5,15(22),16,18(21)-heptaene (34 mg, 0.08 mmol) in methanol (900 μL) and water (150 μL) was added p-toluenesulfonic acid monohydrate (76 mg, 0.4 mmol). The reaction mixture was stirred at 80° C. for 16 h. The reaction mixture was diluted with ethyl acetate and a saturated aqueous NaHCO₃ solution. The aqueous layer was extracted with ethyl acetate. The combined organic layers were washed with brine, dried over anhydrous sodium sulfate, filtered and evaporated under reduced pressure. The resulting solid was recrystallized from acetonitrile, filtered and dried under reduced pressure to afford (8R,13S)-8,13-dimethyl-7,11,14-trioxa-4,19,20-triazatetracyclo[13.5.2.1^{2,6}.0^{18,21}] tricosa-1(20),2(23),3,5,15(22),16,18(21)-heptaene as a solid.

LCMS method F: [M+H]⁺=340.3, t_{R}=2.13 min

LCMS method G: [M+H]⁺=340.3, t_{R}=2.49 min

¹H NMR (400 MHz, d6-DMSO) 13.27-13.25 (1H, m), 8.84 (1H, s), 8.49 (1H, d, J=1.7 Hz), 8.18-8.16 (2H, m), 7.50-7.45 (1H, m), 7.04 (1H, dd, J=2.1, 8.9 Hz), 4.92-4.85 (1H, m), 4.57-4.50 (1H, m), 3.77 (1H, dd, J=7.0, 10.8 Hz), 3.69-3.54 (3H, m), 2.38-2.31 (1H, m), 1.67-1.60 (1H, m), 1.48 (3H, d, J=5.9 Hz), 1.35 (3H, d, J=6.6 Hz) ppm.

### Example 19: (13S)-13-methyl-4-(pyrrolidin-1-yl)-7,11,14-trioxa-5,19,20,23-tetraazatetra cyclo[13.5.2.1^{2,6}.0^{18,21}]tricosa-1(20),2(23),3,5,15(22),16,18(21)heptaene

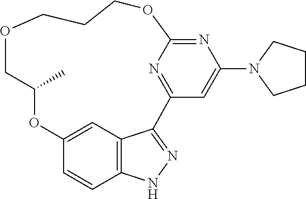 [SMILES: C[C@H]1COCCCOc2nc(cc(N3CCCC3)n2)-c2n[nH]c3ccc(cc23)O1]

Example 19 is prepared according to the synthesis route described in general Scheme E.

### Preparation of Intermediate 116: 3-[(2R)-2-benzyloxypropoxy]propoxy-tert-butyl-dimethyl-silane

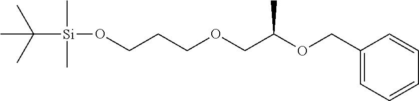 [SMILES: C[C@H](COCCCO[Si](C)(C)C(C)(C)C)OCc1ccccc1]

To a solution of (R)-2-(benzyloxy)propan-1-ol (intermediate 87) (7.1 g, 42.71 mmol) in dry THF (130 mL) at 0° C. under nitrogen atmosphere was added sodium hydride (60% dispersion in mineral oil) (5.12 g, 128.14 mmol). The reaction mixture was stirred at 0° C. for 1 h then (3-bromopropoxy)-tert-butyldimethylsilane (11.9 mL, 51.25 mmol) in dry THF (85 mL) was added and the mixture was stirred at 80° C. for 15 h. The reaction mixture was cooled to 0° C., diluted with ethyl acetate and quenched with a saturated aqueous NH₄Cl solution. The organic layer was washed with brine, dried over anhydrous magnesium sulfate, filtered and evaporated under reduced pressure. The residue was purified by silica gel column chromatography using a heptane/ethyl acetate 99:1 to 90:10 as eluent to afford 3-[(2R)-2-benzyloxypropoxy]propoxy-tert-butyl-dimethyl-silane as a colorless oil.

LCMS method B: [M+H]⁺=339.0, t_{R}=1.469 min

### Preparation of Intermediate 117: (2R)-1-(3-((tert-butyldimethylsilyl)oxy)propoxy) propan-2-ol

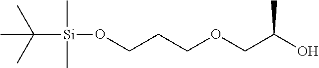 [SMILES: C[C@@H](O)COCCCO[Si](C)(C)C(C)(C)C]

To a solution of 3-[(2R)-2-benzyloxypropoxy]propoxy-tert-butyl-dimethyl-silane (4.5 g, 13.29 mmol) in ethyl acetate (133 mL) was added palladium 10% w on carbon (900 mg). The reaction mixture was stirred under hydrogen atmosphere at RT for 63 h. The reaction mixture was filtered over a pad of celite, rinsed with ethyl acetate and the solvent was removed under reduced pressure. The residue was purified silica gel column chromatography using dichloromethane/methanol 100/0 to 90/10 as eluent to afford (2R)-1-(3-((tert-butyldimethylsilyl)oxy)propoxy)propan-2-ol as a colorless oil.

LCMS method B: [M+H]⁺=249.1, t_{R}=Not detected by UV

### Preparation of Intermediate 118: [(1R)-2-[3-[tert-butyl(dimethyl)silyl]oxypropoxy]-1-methyl-ethyl]methanesulfonate

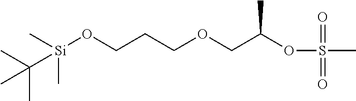 [SMILES: C[C@H](COCCCO[Si](C)(C)C(C)(C)C)OS(C)(=O)=O]

To a solution of (2R)-1-(3-((tert-butyldimethylsilyl)oxy)propoxy)propan-2-ol (220 mg, 0.886 mmol) in dichloromethane (9 mL) at 0° C. under nitrogen atmosphere was added triethylamine (185 μL, 1.329 mmol) and methanesulfonyl chloride (89 μL, 1.152 mmol). The reaction mixture was stirred for 1 h form 0° C. to RT. The reaction mixture was diluted with dichloromethane and washed with a saturated aqueous NaHCO₃ solution. The aqueous phase was extracted with dichloromethane and the combined organic layers were washed with brine, dried over anhydrous magnesium sulfate, filtered and evaporated under reduced pressure to afford [(1R)-2-[3-[tert-butyl(dimethyl)silyl]oxypropoxy]-1-methyl-ethyl]methanesulfonate as a colorless oil. The product was used in the next step without further purification.

LCMS method B: [M+H]⁺=327.0, t_{R}=Not detected by UV

### Preparation of Intermediate 119: 5-((tert-butyldimethylsilyl)oxy)-3-(6-chloro-2-(methylthio)pyrimidin-4-yl)-1-(tetrahydro-2H-pyran-2-yl)-1H-indazole

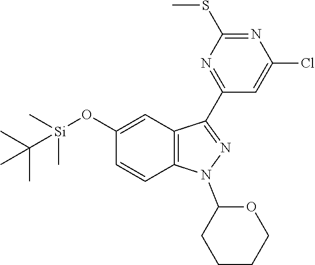 [SMILES: CSc1nc(Cl)cc(-c2nn(C3CCCCO3)c3ccc(O[Si](C)(C)C(C)(C)C)cc23)n1]

Tetrakis(triphenylphosphine)palladium(0) (756 mg, 0.654 mmol) and XPhos (624 mg, 1.309 mmol) were added to a mixture of 5-((tert-butyldimethylsilyl)oxy)-1-(tetrahydro-2H-pyran-2-yl)-3-(4,4,5,5-tetramethyl-1,3,2-dioxaborolan-2-yl)-1H-indazole and (5-((tert-butyldimethyl silyl)oxy)-1-(tetrahydro-2H-pyran-2-yl)-1H-indazol-3-yl)boronic acid (6 g, 13.086 mmol), 4,6-dichloro-2-(methylthio)pyrimidine (2.55 g 13.08 mmol) and triethylamine (5.47 mL, 39.25 mmol) in dioxane (72 mL) and water (24 mL). The reaction mixture was degassed with N₂ for 5 min and stirred at 900 for 1 h. The reaction mixture was diluted with ethyl acetate and water was added. The aqueous layer was extracted with ethyl acetate. The combined organic layers were washed with brine, dried over anhydrous magnesium sulfate, filtered and evaporated under reduced pressure. The residue was purified by silica gel column chromatography using heptane/ethyl acetate 100/0 to 90/10 as eluent. The resulting solid was triturated with heptane, filtered and dried to afford 5-((tert-butyldimethylsilyl)oxy)-3-(6-chloro-2-(methylthio) pyrimidin-4-yl)-1-(tetrahydro-2H-pyran-2-yl)-1H-indazole as a white solid.

LCMS method B: [M+H]⁺=491.0-493.0, t_{R}=1.691 min

### Preparation of Intermediate 120: 5-((tert-butyldimethylsilyl)oxy)-3-(2-(methylthio)-6-(pyrrolidin-1-yl)pyrimidin-4-yl)-1-(tetrahydro-2H-pyran-2-yl)-1H-indazole

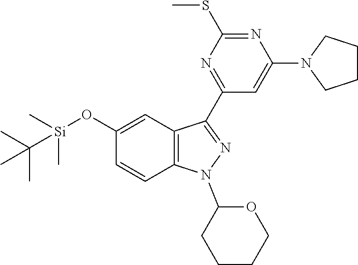 [SMILES: CSc1nc(-c2nn(C3CCCCO3)c3ccc(O[Si](C)(C)C(C)(C)C)cc23)cc(N2CCCC2)n1]

To a solution of 5-((tert-butyldimethylsilyl)oxy)-3-(6-chloro-2-(methylthio)pyrimidin-4-yl)-1-(tetrahydro-2H-pyran-2-yl)-1H-indazole (1 g, 2.036 ml) in dioxane (15 mL) was added pyrrolidine (256 μl, 3.054 mmol). The reaction mixture was heated at 100° C. for 16 h. The solvent was evaporated under reduced pressure. The residue was purified by silica gel column chromatography using heptane/ethyl acetate 100/0 to 85/15 as eluent to afford 5-((tert-butyldimethylsilyl)oxy)-3-(2-(methylthio)-6-(pyrrolidin-1-yl)pyrimidin-4-yl)-1-(tetrahydro-2H-pyran-2-yl)-1H-indazole as a colorless foam.

LCMS method B: [M+H]⁺=526.0, t_{R}=1.679 min

### Preparation of Intermediate 121: 3-(2-(methylthio)-6-(pyrrolidin-1-yl)pyrimidin-4-yl)-1-(tetrahydro-2H-pyran-2-yl)-1H-indazol-5-ol

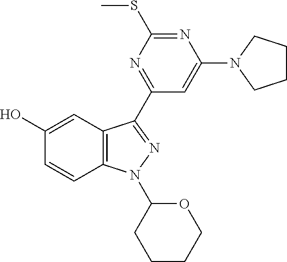 [SMILES: CSc1nc(-c2nn(C3CCCCO3)c3ccc(O)cc23)cc(N2CCCC2)n1]

To a solution of 5-((tert-butyldimethylsilyl)oxy)-3-(2-(methylthio)-6-(pyrrolidin-1-yl) pyrimidin-4-yl)-1-(tetrahydro-2H-pyran-2-yl)-1H-indazole (431 mg, 0.82 mmol) in THF (5 mL) at 0° C. was added TBAF (1M solution in THF) (1.23 mL, 1.23 mmol). The reaction mixture was stirred at RT for 15 h. The reaction mixture was diluted with ethyl acetate and washed with a saturated aqueous NaHCO₃ solution then brine. The organic layer was dried over anhydrous magnesium sulfate, filtered and evaporated under reduced pressure. The residue was purified by silica gel column chromatography using a heptane/ethyl acetate 100/0 to 80/20 as eluent to afford 3-(2-(methylthio)-6-(pyrrolidin-1-yl)pyrimidin-4-yl)-1-(tetrahydro-2H-pyran-2-yl)-1H-indazol-5-ol as a white solid.

LCMS method B: [M+H]⁺=412.0, t_{R}=0.976 min

LCMS method E: [M+H]⁺=412.1, t_{R}=3.210 min

### Preparation of Intermediate 122: tert-butyl-dimethyl-[3-[(2S)-2-[3-(2-methyl sulfanyl-6-pyrrolidin-1-yl-pyrimidin-4-yl)-1-tetrahydropyran-2-yl-indazol-5-yl]oxypropoxy]propoxy]silane

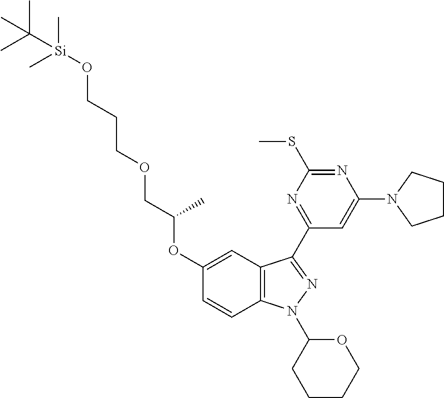 [SMILES: CSc1nc(-c2nn(C3CCCCO3)c3ccc(O[C@@H](C)COCCCO[Si](C)(C)C(C)(C)C)cc23)cc(N2CCCC2)n1]

To a solution of 3-(2-(methylthio)-6-(pyrrolidin-1-yl)pyrimidin-4-yl)-1-(tetrahydro-2H-pyran-2-yl)-1H-indazol-5-ol (300 mg, 0.729 mmol) in DMF (4 mL) was added cesium carbonate (356 mg, 1.093 mmol) and [(1R)-2-[3-[tert-butyl(dimethyl)silyl]oxypropoxy]-1-methyl-ethyl] methanesulfonate (286 mg, 0.875 mmol). The reaction mixture was stirred at RT for 15 h. The mixture was diluted with ethyl acetate and washed with brine. The organic layer was dried over anhydrous magnesium sulfate, filtered and evaporated under reduced pressure. The residue was purified by silica gel column chromatography using heptane/ethyl acetate 100/0 to 30/70 as eluent. to afford tert-butyl-dimethyl-[3-[(2S)-2-[3-(2-methylsulfanyl-6-pyrrolidin-1-yl-pyrimidin-4-yl)-1-tetrahydropyran-2-yl-indazol-5-yl]oxypropoxy]propoxy]silane as a colorless oil.

LCMS method B: [M+H]⁺=642.0, t_{R}=1.933 min

### Preparation of Intermediate 123: 3-((2S)-2-((3-(2-(methylthio)-6-(pyrrolidin-1-yl) pyrimidin-4-yl)-1-(tetrahydro-2H-pyran-2-yl)-1H-indazol-5-yl)oxy)propoxy)propan-1-ol

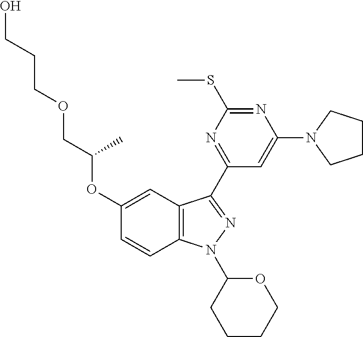 [SMILES: CSc1nc(-c2nn(C3CCCCO3)c3ccc(O[C@@H](C)COCCCO)cc23)cc(N2CCCC2)n1]

To a solution of tert-butyl-dimethyl-[3-[(2S)-2-[3-(2-methylsulfanyl-6-pyrrolidin-1-yl-pyrimidin-4-yl)-1-tetrahydropyran-2-yl-indazol-5-yl]oxypropoxy] propoxy]silane (300 mg, 0.467 mmol) in THF (2 mL) at 0° C. was added TBAF (1M solution in THF) (701 μL, 0.701 mmol). The reaction mixture was stirred at RT for 15 h. The reaction mixture was diluted with ethyl acetate, washed with a saturated aqueous NaHCO₃ solution and brine. The organic layer was dried over anhydrous magnesium sulfate, filtered and evaporated under reduced pressure. The residue was purified by silica gel column chromatography using heptane/ethyl acetate 100/0 to 80/20 as eluent to afford 3-((2S)-2-((3-(2-(methylthio)-6-(pyrrolidin-1-yl)pyrimidin-4-yl)-1-(tetrahydro-2H-pyran-2-yl)-1H-indazol-5-yl)oxy)propoxy)propan-1-ol as a yellow oil.

LCMS method B: [M+H]⁺=528.0, t_{R}=1.179 min

### Preparation of Intermediate 124: 3-((2S)-2-((3-(2-(methylsulfonyl)-6-(pyrrolidin-1-yl)pyrimidin-4-yl)-1-(tetrahydro-2H-pyran-2-yl)-1H-indazol-5-yl)oxy)propoxy) propan-1-ol

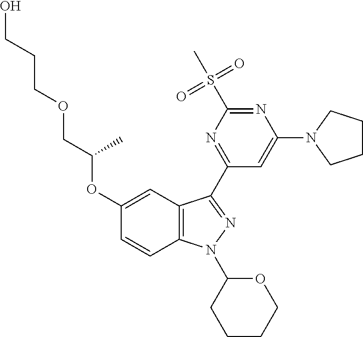 [SMILES: C[C@@H](COCCCO)Oc1ccc2c(c1)c(-c1cc(N3CCCC3)nc(S(C)(=O)=O)n1)nn2C1CCCCO1]

To a solution of 3-((2S)-2-((3-(2-(methylthio)-6-(pyrrolidin-1-yl)pyrimidin-4-yl)-1-(tetrahydro-2H-pyran-2-yl)-1H-indazol-5-yl)oxy)propoxy)propan-1-ol (222 mg, 0.421 mmol) in dichloromethane (4 mL) at 0° C. was added 3-chloroperbenzoic acid (242 mg, 1.053 mmol). The reaction mixture was stirred at RT for 2 h. The reaction mixture was diluted with dichloromethane and washed with a saturated aqueous NaHCO₃ solution. The aqueous layer was extracted with dichloromethane and the combined organic layers were washed with brine, dried over anhydrous magnesium sulfate, filtered and evaporated under reduced pressure. The residue was purified by silica gel column chromatography using heptane/ethyl acetate 100/0 to 20/80 as eluent to afford 3-((2S)-2-((3-(2-(methylsulfonyl)-6-(pyrrolidin-1-yl)pyrimidin-4-yl)-1-(tetrahydro-2H-pyran-2-yl)-1H-indazol-5-yl)oxy)propoxy)propan-1-ol as a yellow solid.

LCMS method A: [M+H]⁺=560.0, t_{R}=1.025 min

### Preparation of Intermediate 125: (13S)-13-methyl-19-(oxan-2-yl)-4-(pyrrolidin-1-yl)-7,11,14-trioxa-5,19,20,23-tetraazatetracyclo[13.5.2.1^{2,6}.0^{18,21}]tricosa-1(20),2(23), 3,5,15(22),16,18(21)-heptaene

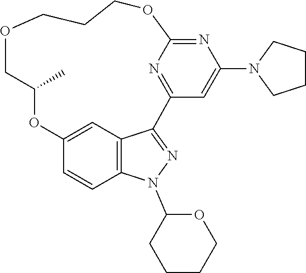 [SMILES: C[C@H]1COCCCOc2nc(cc(N3CCCC3)n2)-c2nn(C3CCCCO3)c3ccc(cc23)O1]

A solution of 3-((2S)-2-((3-(2-(methylsulfonyl)-6-(pyrrolidin-1-yl)pyrimidin-4-yl)-1-(tetrahydro-2H-pyran-2-yl)-1H-indazol-5-yl)oxy)propoxy)propan-1-ol (132 mg, 0.236 mmol) in dry THF (12 mL) was added dropwise under nitrogen atmosphere to stirred solution of sodium hydride (60% dispersion in mineral oil) (30 mg, 0.708 mmol) in dry THF (12 mL). The reaction mixture was stirred at RT for 1 h. The reaction mixture was cooled to 0° C., diluted with ethyl acetate and quenched with a saturated aqueous NH₄Cl solution. The organic layer was washed with brine, dried over anhydrous magnesium sulfate, filtered and evaporated under reduced pressure. The residue was purified by silica gel column chromatography using heptane/ethyl acetate 100/0 to 60/40 as eluent to afford (13S)-13-methyl-19-(oxan-2-yl)-4-(pyrrolidin-1-yl)-7,11,14-trioxa-5,19,20,23-tetraazatetracyclo[13.5.2.1^{2,6}.0^{18,21}]tricosa-1(20), 2(23),3,5,15(22),16,18(21)-heptaene as a yellow oil.

LCMS method B: [M+H]⁺=480.0, t_{R}=1.273 min

### Preparation of Example 19: (13S)-13-methyl-4-(pyrrolidin-1-yl)-7,11,14-trioxa-5,19,20,23-tetraazatetracyclo[13.5.2.1^{2,6}.0^{18,21}]tricosa-1(20),2(23),3,5,15(22),16, 18(21)heptaene

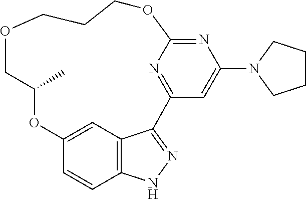 [SMILES: C[C@H]1COCCCOc2nc(cc(N3CCCC3)n2)-c2n[nH]c3ccc(cc23)O1]

HCl (4M in dioxane) (10.45 mL) was added to (13S)-13-methyl-19-(oxan-2-yl)-4-(pyrrolidin-1-yl)-7,11,14-trioxa-5,19,20,23-tetraazatetracyclo[13.5.2.1^{2,6}.0^{18,21}]tricosa-1(20),2(23),3,5, 15(22),16,18(21)-heptaene (100 mg, 0.209 mmol). The reaction mixture was stirred at RT for 15 h. The solvent was evaporated under reduced pressure and the residue was basified to pH=8 using a saturated aqueous NaHCO₃ solution. The aqueous layer was extracted with dichloromethane. The combined organic layers were dried over anhydrous magnesium sulfate, filtered and evaporated under reduced pressure. The residue was purified by silica gel column chromatography using heptane/ethyl acetate 100/0 to 50/50/as eluent to afford (13S)-13-methyl-4-(pyrrolidin-1-yl)-7,11,14-trioxa-5,19,20,23-tetraazatetracyclo[13.5.2.1^{2,6}.0^{18,21}] tricosa-1(20),2(23),3,5,15(22),16,18(21)heptaene as a solid.

LCMS method E: [M+H]⁺=396.2, t_{R}=3.328 min

LCMS method D: [M+H]⁺=396.2, t_{R}=3.858 min

¹H NMR (400 MHz, d6-DMSO) 13.32 (s, 1H), 8.71 (d, J=2.4 Hz, 1H), 7.44 (d, J=8.9 Hz, 1H), 7.00 (dd, J=8.9, 2.4 Hz, 1H), 6.85 (s, 1H), 5.10 (td, J=11.5, 4.7 Hz, 1H), 4.31 (qd, J=6.4, 3.9 Hz, 1H), 4.22 (td, J=11.5, 4.8 Hz, 1H), 3.78 (dd, J=9.7, 6.2 Hz, 1H), 3.59 (qd, J=10.3, 4.3 Hz, 2H), 3.46 (brs, 4H), 3.40 (dd, J=9.7, 3.9 Hz, 1H), 2.42-2.28 (m, 1H), 1.95 (brs, 4H), 1.77 (d, J=13.0 Hz, 1H), 1.32 (d, J=6.4 Hz, 3H) ppm.

### Example 20: (8S,13S)-8,13-dimethyl-7,10,14-trioxa-4,19,20-triazatetracyclo[13.5.2.1^{2,6}. 0^{18,21}]tricosa-1(20),2(23),3,5,15(22),16,18(21)-heptaene

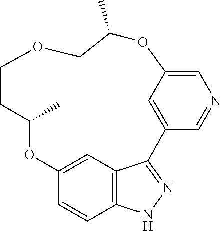 [SMILES: C[C@H]1CCOC[C@H](C)Oc2cncc(c2)-c2n[nH]c3ccc(cc23)O1]

Example 20 is prepared according to the synthesis route described in general Scheme D.

### Preparation of Intermediate 126: (2R)-4-trityloxybutan-2-ol

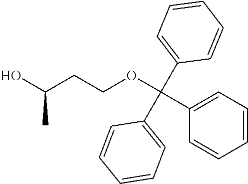 [SMILES: C[C@@H](O)CCOC(c1ccccc1)(c1ccccc1)c1ccccc1]

To solution of (3R)-butane-1,3-diol (3 g, 33.29 mmol) in dichloromethane (30 mL) at 0° C. was added triethylamine (2 mL, 14.43 mmol) followed by a dropwise addition of trityl chloride (9.28 g, 33.29 mmol). The reaction mixture was stirred at RT overnight. Water was added to the suspension and the phases were separated. The aqueous layer was extracted with dichloromethane and the combined organic layers were washed with brine, dried over anhydrous magnesium sulfate, filtered and evaporated under reduced pressure. The residue was purified by silica gel column chromatography using cyclohexane/ethyl acetate 95/5 to 80/20 as eluent to afford (2R)-4-trityloxybutan-2-ol as a colorless oil.

LCMS method F: [M−H]⁺=not detected, t_{R}=3.05 min

### Preparation of Intermediate 127: [[(3R)-3-benzyloxybutoxy]-diphenyl-methyl] benzene

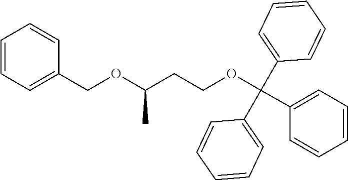 [SMILES: C[C@H](CCOC(c1ccccc1)(c1ccccc1)c1ccccc1)OCc1ccccc1]

To a solution of (2R)-4-trityloxybutan-2-ol (7 g, 21 mmol) in DMF (40 mL) at 0° C. was added portionwise sodium hydride (60% dispersion in mineral oil) (1.26 g, 31.58 mmol). The reaction mixture was stirred for 20 min. A solution of benzyl bromide (3.74 mL, 31.5 mmol) in DMF (5 mL) was added dropwise and the suspension was stirred at RT for 16 h. The reaction mixture was quenched by water the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 90/10 as eluent to afford [[(3R)-3-benzyloxybutoxy]-diphenyl-methyl]benzene as a colorless oil.

LCMS method F: [M+Na]⁺=445.2, t_{R}=3.75 min

### Preparation of Intermediate 128: (3R)-3-benzyloxybutan-1-ol

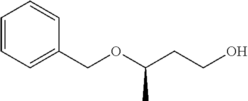

A solution of [[(3R)-3-benzyloxybutoxy]-diphenyl-methyl]benzene (7 g, 16.56 mmol) in a mixture of water (19.8 mL), acetic acid (26.4 mL) and methanol (19.8 mL) was stirred at 50° C. for 20 h then 48 h at RT. The suspended solid was filtered, washed with water, cyclohexane and discarded. The filtrate was extracted with ethyl acetate and the combined organic layers were washed with brine, dried over anhydrous magnesium sulfate, filtered and evaporated under reduced pressure. The residue was purified by silica gel column chromatography using cyclohexane/ethyl acetate 95/5 to 65/35 as eluent to afford (3R)-3-benzyloxybutan-1-ol as a colorless liquid.

LCMS method F: [M+H]⁺=not detected, t_{R}=1.89 min

### Preparation of Intermediate 129: [(3R)-3-benzyloxybutyl] 4-methylbenzenesulfonate

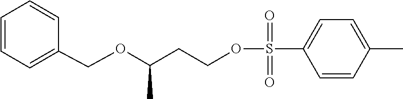 [SMILES: Cc1ccc(S(=O)(=O)OCC[C@@H](C)OCc2ccccc2)cc1]

To a solution of (3R)-3-benzyloxybutan-1-ol (1.3 g, 7.21 mmol) in pyridine (10 mL) at 0° C. was added p-toluenesulfonyl chloride (1.512 g, 7.93 mmol). The reaction mixture was allowed to warm up to RT and stirred at RT for 3 h. The solvent was evaporated under reduced pressure. The residue was diluted with a saturated NH₄Cl solution and ethyl acetate. The layers were separated. The aqueous layer was extracted with ethyl acetate and the combined organic layers were dried over anhydrous sodium sulfate, filtered and evaporated under reduced pressure to give [(3R)-3-benzyloxybutyl]4-methylbenzenesulfonate as a white solid which was used in the next step without further purification.

LCMS method F: [M+H]⁺=335.1, t_{R}=3.03 min

### Preparation of Intermediate 130: methyl (2R)-2-tetrahydropyran-2-yloxypropanoate

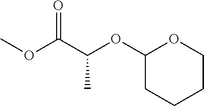

To a solution of methyl (2R)-2-hydroxypropanoate (6.3 g, 60.52 mmol) in diethylether (30 mL) was added (1S)-camphor-10-sulfonic acid (703 mg, 3.03 mmol) and 3,4-dihydro-2H-pyran (6.63 mL, 72.62 mmol). The reaction mixture was stirred at RT for 5 h. Water was added and the layers were separated. The organic layer was washed with a saturated aqeuous NaHCO₃ solution, dried over anhydrous sodium sulfate, filtered and evaporated under reduced pressure to afford methyl (2R)-2-tetrahydropyran-2-yloxypropanoate as a yellow oil which was used in the next step without further purification.

¹H NMR (400 MHz, CDCl₃), 4.74-4.69 (1H, m), 4.45 (1H, q, J=7.0 Hz), 4.05-3.83 (2H, m), 3.75 (3H, s), 3.57-3.46 (2H, m), 1.92-1.52 (4H, m), 1.47 (3H, d, J=7.0 Hz) ppm.

### Preparation of Intermediate 131: (2R)-2-tetrahydropyran-2-yloxypropan-1-ol

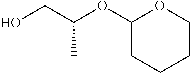

To a suspension of lithium borohydride (2.41 g, 63.55 mmol) in dry THF (75 mL) at 0° C. was added dropwise methyl (2R)-2-tetrahydropyran-2-yloxypropanoate (11.39 g, 60.52 mmol) in dry THF (45 mL). The reaction mixture was stirred at 0° C. for 1 h and at RT overnight. Cold water was added. The reaction mixture was filtered, washed with ethyl acetate and the layers were separated. The aqueous layer was extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 50/50 as eluent to afford (2R)-2-tetrahydropyran-2-yloxypropan-1-ol as a colorless oil.

¹H NMR (400 MHz, CDCl₃), 4.75 (1H, dd, J=2.7, 5.1 Hz), 4.01-3.84 (1H, m), 3.65-3.45 (2H, m), 2.16 (1H, brs), 1.90-1.53 (8H, m), 1.24 (3H, d, J=6.5 Hz) ppm.

### Preparation of Intermediate 132: 2-[(1R)-2-[(3R)-3-benzyloxybutoxy]-1-methyl-ethoxy]tetrahydropyran

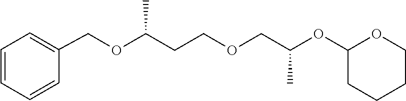 [SMILES: C[C@H](CCOC[C@@H](C)OC1CCCCO1)OCc1ccccc1]

To a solution of (2R)-2-tetrahydropyran-2-yloxypropan-1-ol (316 mg, 1.97 mmol) in dry DMF (12 mL) at 0° C. was added sodium hydride (60% dispersion in mineral oil) (118 mg, 2.95 mmol). The reaction mixture was stirred at 0° C. for 10 min. A solution of [(3R)-3-benzyloxybutyl] 4-methylbenzenesulfonate (792 mg, 2.37 mmol) in dry DMF (5 mL) was added dropwise. The reaction was allowed to warm up to RT and the mixture was stirred at RT for 30 min. The mixture was stirred at 50° C. for 1 h and at 70° C. for 3 h. The mixture was cooled to RT and quenched with water and ethyl acetate was added. The layers were separated. The aqueous layer was extracted with ethyl acetate and the combined organic layers were dried over anhydrous sodium sulfate, filtered and evaporated under reduced pressure. The residue was purified by silica gel column chromatography using cyclohexane/ethyl acetate 100/0 to 80/20 as eluent to afford 2-[(1R)-2-[(3R)-3-benzyloxybutoxy]-1-methyl-ethoxy]tetrahydropyran as a colorless oil.

LCMS method F: [M+H]⁺=not detected, t_{R}=3.08 min

### Preparation of Intermediate 133: (2R)-1-[(3R)-3-benzyloxybutoxy]propan-2-ol

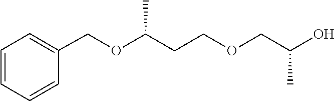 [SMILES: C[C@H](CCOC[C@@H](C)O)OCc1ccccc1]

To a solution of 2-[(1R)-2-[(3R)-3-benzyloxybutoxy]-1-methyl-ethoxy]tetrahydropyran (380 mg, 1.18 mmol) in methanol (15 mL) and water (3 mL) was added p-toluenesulfonic acid monohydrate (1.121 g, 5.89 mmol). The reaction mixture was stirred at 65° C. for 1 h. The solvent was removed under reduced pressure. The residue was diluted with a saturated aqueous NaHCO₃ solution and ethyl acetate. The layers were separated. The aqueous layer was extracted with ethyl acetate and the combined organic layers were dried over anhydrous sodium sulfate, filtered and evaporated under reduced pressure to afford (2R)-1-[(3R)-3-benzyloxybutoxy]propan-2-ol as a colorless oil which was used in the next step without further purification.

LCMS method F: [M+H]⁺=239.3, t_{R}=2.26 min

### Preparation of Intermediate 134: 3-[(1S)-2-[(3R)-3-benzyloxybutoxy]-1-methyl-ethoxy]-5-bromo-pyridine

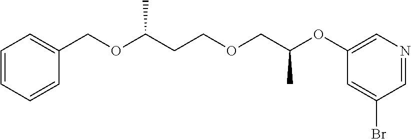 [SMILES: C[C@H](CCOC[C@H](C)Oc1cncc(Br)c1)OCc1ccccc1]

To a solution of (2R)-1-[(3R)-3-benzyloxybutoxy]propan-2-ol (258 mg, 1.08 mmol) in dry THF (3.5 mL) was added under argon atmosphere 5-bromopyridin-3-ol (171 mg, 0.98 mmol), triphenylphosphine (385 mg, 1.47 mmol) and dropwise DIAD (290 μL, 1.47 mmol). The reaction mixture was stirred at RT for 1 h. The solvent was evaporated under reduced pressure. The residue was purified by silica gel column chromatography using cyclohexane/ethyl acetate 100/0 to 75/25 as eluent to afford 3-[(1S)-2-[(3R)-3-benzyloxybutoxy]-1-methyl-ethoxy]-5-bromo-pyridine as a pale yellow oil.

LCMS method F: [M+H]⁺=394.1-396.1, t_{R}=3.14 min

### Preparation of Intermediate 135: [3-[5-[(1S)-2-[(3R)-3-benzyloxybutoxy]-1-methyl-ethoxy]-3-pyridyl]-1-tetrahydropyran-2-yl-indazol-5-yl]oxy-tert-butyl-dimethyl-silane

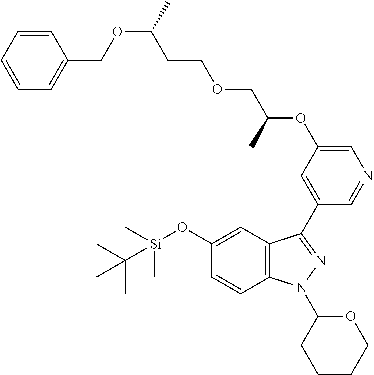 [SMILES: C[C@H](CCOC[C@H](C)Oc1cncc(-c2nn(C3CCCCO3)c3ccc(O[Si](C)(C)C(C)(C)C)cc23)c1)OCc1ccccc1]

To a suspension of 3-[(1S)-2-[(3R)-3-benzyloxybutoxy]-1-methyl-ethoxy]-5-bromo-pyridine (342 mg, 0.87 mmol) in dioxane and water 20:1 (4.2 mL) were added tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (477 mg, 1.04 mmol), tetrakis(triphenylphosphine)palladium(0) (20 mg, 0.02 mmol), Xphos (17 mg, 0.03 mmol) and potassium phosphate tribasic (552 mg, 2.60 mmol). The reaction mixture was degassed by bubbling under argon for 15 min and was stirred at 90° C. for 1 h. Additional tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (80 mg, 0.17 mmol) was added and the reaction mixture was stirred at 90° C. for 3 h. The mixture was cooled to RT and the solvent was evaporated under reduced pressure. The residue was purified on silica gel column chromatography using cyclohexane/ethyl acetate 100/0 to 60/40 as eluent to afford [3-[5-[(1S)-2-[(3R)-3-benzyloxybutoxy]-1-methyl-ethoxy]-3-pyridyl]-1-tetrahydropyran-2-yl-indazol-5-yl]oxy-tert-butyl-dimethyl-silane as a pink oil.

LCMS method F: [M+H]⁺=646.4, t_{R}=3.94 min

### Preparation of Intermediate 136: (2R)-4-[(2S)-2-[[5-[5-[tert-butyl(dimethyl)silyl]oxy-1-tetrahydropyran-2-yl-indazol-3-yl]-3-pyridyl]oxy]propoxy]butan-2-ol

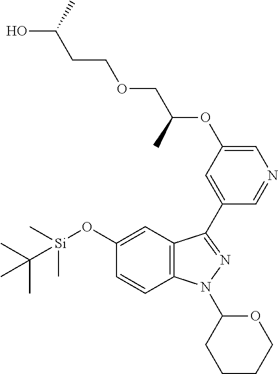 [SMILES: C[C@@H](O)CCOC[C@H](C)Oc1cncc(-c2nn(C3CCCCO3)c3ccc(O[Si](C)(C)C(C)(C)C)cc23)c1]

To a suspension of [3-[5-[(1S)-2-[(3R)-3-benzyloxybutoxy]-1-methyl-ethoxy]-3-pyridyl]-1-tetrahydropyran-2-yl-indazol-5-yl]oxy-tert-butyl-dimethyl-silane (229 mg, 0.35 mmol) in ethanol (2.5 mL) was added under argon atmosphere palladium on charcoal 10% (23 mg). The reaction mixture was stirred under hydrogen atmosphere at RT overnight. The mixture was filtered over a pad of celite and washed with methanol and ethyl acetate. The filtrate was evaporated under reduced pressure to give (2R)-4-[(2S)-2-[[5-[5-[tert-butyl(dimethyl) silyl]oxy-1-tetrahydropyran-2-yl-indazol-3-yl]-3-pyridyl]oxy]propoxy]butan-2-ol as a pale brown oil which was used in the next step without further purification.

LCMS method F: [M+H]⁺=556.3, t_{R}=3.40 min

### Preparation of Intermediate 137: [(1R)-3-[(2S)-2-[[5-[5-[tert-butyl(dimethyl)silyl] oxy-1-tetrahydropyran-2-yl-indazol-3-yl]-3-pyridyl]oxy]propoxy]-1-methyl-propyl] methanesulfonate

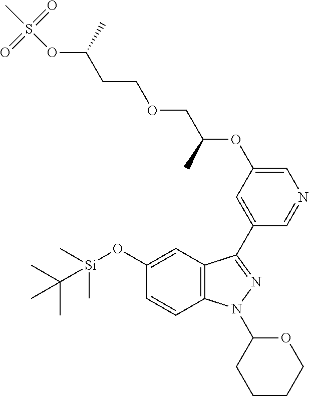 [SMILES: C[C@H](CCOC[C@H](C)Oc1cncc(-c2nn(C3CCCCO3)c3ccc(O[Si](C)(C)C(C)(C)C)cc23)c1)OS(C)(=O)=O]

To a solution of (2R)-4-[(2S)-2-[[5-[5-[tert-butyl(dimethyl)silyl]oxy-1-tetrahydropyran-2-yl-indazol-3-yl]-3-pyridyl]oxy]propoxy]butan-2-ol (197 mg, 0.35 mmol) in dichloromethane (1.8 mL) at 0° C. were added triethylamine (73 μL, 0.52 mmol) and methanesulfonyl chloride (32 μL, 0.42 mmol). The reaction mixture was stirred at 0° C. for 10 min and at RT for 1 h. Additional triethylamine (73 μL, 0.52 mmol) and methanesulfonyl chloride (32 μL, 0.42 mmol) were added. The reaction was stirred at RT for 1 h. Additional triethylamine (73 μL, 0.52 mmol) and methanesulfonyl chloride (32 μL, 0.42 mmol) were added and the reaction was stirred at RT for 2 h. Water was added and the layers were separated. The aqueous layer was extracted with dichloromethane and the combined organic layers were dried over anhydrous sodium sulfate, filtered and evaporated reduced pressure to afford [(1R)-3-[(2S)-2-[[5-[5-[tert-butyl(dimethyl)silyl]oxy-1-tetrahydropyran-2-yl-indazol-3-yl]-3-pyridyl]oxy]propoxy]-1-methyl-propyl] methanesulfonate as a yellow oil which was used in the next step without further purification.

LCMS method F: [M+H]⁺=634.3, t_{R}=3.51 min

### Preparation of Intermediate 138: [(1R)-3-[(2S)-2-[[5-(5-hydroxy-1-tetrahydropyran-2-yl-indazol-3-yl)-3-pyridyl]oxy]propoxy]-1-methyl-propyl]methanesulfonate

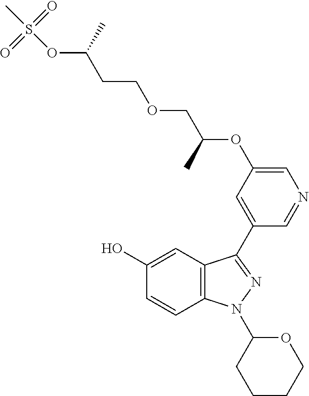 [SMILES: C[C@H](CCOC[C@H](C)Oc1cncc(-c2nn(C3CCCCO3)c3ccc(O)cc23)c1)OS(C)(=O)=O]

To a solution of [(1R)-3-[(2S)-2-[[5-[5-[tert-butyl(dimethyl)silyl]oxy-1-tetrahydropyran-2-yl-indazol-3-yl]-3-pyridyl]oxy]propoxy]-1-methyl-propyl] methanesulfonate (224 mg, 0.35 mmol) in THF (1.8 mL) at 0° C. was added TBAF (1M solution in THF) (0.38 mL, 0.38 mmol). The reaction mixture was stirred at RT for 30 min. Additional TBAF (1M solution in THF) (0.19 mL, 0.19 mmol) was added and the reaction mixture was stirred at RT for 2 h. The solvent was removed under reduced pressure to afford [(1R)-3-[(2S)-2-[[5-(5-hydroxy-1-tetrahydropyran-2-yl-indazol-3-yl)-3-pyridyl]oxy]propoxy]-1-methyl-propyl]methane sulfonate as an orange oil which was used in the next step without further purification.

LCMS method F: [M+H]⁺=520.2, t_{R}=2.35 min

### Preparation of Intermediate 139: [3-[5-[(1S)-1-methyl-2-[(3R)-3-methylsulfonyloxy butoxy]ethoxy]-3-pyridyl]-1-tetrahydropyran-2-yl-indazol-5-yl]methanesulfonate

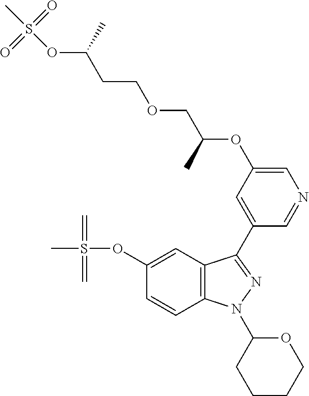 [SMILES: C=S(=C)(C)Oc1ccc2c(c1)c(-c1cncc(O[C@@H](C)COCC[C@@H](C)OS(C)(=O)=O)c1)nn2C1CCCCO1]

To a solution of cesium carbonate (285 mg, 0.87 mmol) in dry DMF (10 mL) heated at 60° C. was added dropwise under argon atmosphere a solution of [(1R)-3-[(2S)-2-[[5-(5-hydroxy-1-tetrahydropyran-2-yl-indazol-3-yl)-3-pyridyl]oxy]propoxy]-1-methyl-propyl]methane sulfonate (182 mg, 0.35 mmol) in dry DMF (10 mL). The reaction mixture was stirred at 60° C. for 15 min. The reaction was cooled to RT and the reaction was diluted with water and ethyl acetate. The layers were separated. The aqueous layer was extracted with ethyl acetate and the combined organic layers were dried over anhydrous sodium sulfate, filtered and evaporated under reduced pressure to afford [3-[5-[(1S)-1-methyl-2-[(3R)-3-methylsulfonyloxybutoxy] ethoxy]-3-pyridyl]-1-tetrahydropyran-2-yl-indazol-5-yl] methanesulfonate as an orange oil which was used in the next step without further purification.

LCMS method F: [M+H]⁺=598.2, t_{R}=2.62 min

### Preparation of Intermediate 140: (8S,13S)-8,13-dimethyl-19-(oxan-2-yl)-7,10,14-trioxa-4,19,20-triazatetracyclo[13.5.2.1^{2,6}.0^{18,21}]tricosa-1(20),2(23),3,5,15(22),16, 18(21)-heptaene

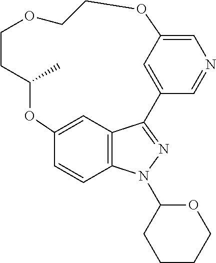 [SMILES: C[C@H]1CCOCCOc2cncc(c2)-c2nn(C3CCCCO3)c3ccc(cc23)O1]

To a solution of [3-[5-[(1S)-1-methyl-2-[(3R)-3-methylsulfonyloxybutoxy]ethoxy]-3-pyridyl]-1-tetrahydropyran-2-yl-indazol-5-yl]methanesulfonate (210 mg, 0.35 mmol) in methanol (7 mL) and THF (7 mL) was added dropwise 1M aqueous NaOH solution (1.75 mL, 1.75 mmol). The reaction mixture was stirred at 50° C. for 5 h. The reaction was cooled to RT and the solvent was evaporated under reduced pressure. The residue was diluted with water and ethyl acetate. The layers were separated. The aqueous layer was extracted with ethyl acetate and the combined organic layers were dried over anhydrous sodium sulfate, filtered and evaporated under reduced pressure. The residue was purified by silica gel column chromatography using cyclohexane/ethyl acetate 100/0 to 50/50 as eluent to afford (8S,13S)-8,13-dimethyl-19-(oxan-2-yl)-7,10,14-trioxa-4,19,20-triazatetracyclo[13.5.2.1^{2,6}.0^{18,21}]tricosa-1(20),2(23),3,5,15(22), 16,18(21)-heptaene as a colorless oil.

LCMS method F: [M+H]⁺=424.4, t_{R}=2.88 min

### Preparation of Example 20: (8S,13S)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene

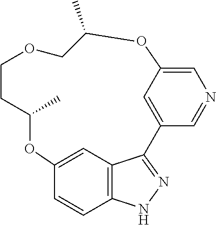 [SMILES: C[C@H]1CCOC[C@H](C)Oc2cncc(c2)-c2n[nH]c3ccc(cc23)O1]

To a solution of (8S,13S)-8,13-dimethyl-19-(oxan-2-yl)-7,10,14-trioxa-4,19,20-triazatetra cyclo[13.5.2.1^{2,6}.0^{18,21}]tricosa-1(20),2(23),3,5,15(22),16,18(21)-heptaene (19 mg, 0.045 mmol) in methanol (0.15 mL) and water (0.025 mL) was added p-toluenesulfonic acid monohydrate (42 mg, 0.22 mmol). The reaction mixture was stirred at 65° C. for 96 h. The reaction mixture was cooled to RT and diluted with a saturated aqueous NaHCO₃ solution and ethyl acetate. The layers were separated. The aqueous layer was extracted with ethyl acetate and the combined organic layers were dried over anhydrous sodium sulfate, filtered and evaporated under reduced pressure to afford (8S,13S)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene as a solid.

LCMS method F: [M+H]⁺=340.3, t_{R}=2.18 min

LCMS method G: [M+H]⁺=340.3, t_{R}=2.47 min

¹H NMR (400 MHz, CDCl3) δ 10.17 (1H, ls), 8.83 (1H, d, J=1.5 Hz), 8.41-8.33 (2H, m), 7.72 (1H, d, J=2.3 Hz), 7.44 (1H, d, J=9.5 Hz), 7.10-7.07 (1H, dd, J=2.3 Hz, J=8.9 Hz), 4.65-4.53 (2H, m), 4.05-4.00 (1H, m), 3.73-3.68 (1H, m), 3.66-3.60 (1H, m), 3.55-3.51 (1H, m), 2.54-2.46 (1H, m), 1.74-1.66 (1H, m), 1.47 (3H, d, J=6.1 Hz), 1.44 (3H, d, J=6.7 Hz) ppm.

### Example 21: (8R,13S)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene

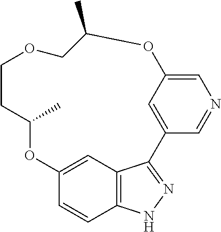 [SMILES: C[C@@H]1COCC[C@H](C)Oc2ccc3[nH]nc(c3c2)-c2cncc(c2)O1]

Example 21 is prepared according to the synthesis route described in general Scheme D, following the same synthesis procedures as for Example 20.

### Preparation of Intermediate 141: (8R,13S)-8,13-dimethyl-19-(oxan-2-yl)-7,10,14-trioxa-4,19,20-triazatetracyclo[13.5.2.1^{2,6}.0^{18,21}]tricosa-1(20),2(23),3,5,15(22),16, 18(21)-heptaene

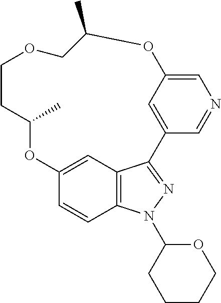 [SMILES: C[C@@H]1COCC[C@H](C)Oc2ccc3c(c2)c(nn3C2CCCCO2)-c2cncc(c2)O1]

To a solution of cesium carbonate (424 mg, 1.3 mmol) in dry DMF (14 mL) heated at 60° C. was added dropwise under argon atmosphere a solution of [(1R)-3-[(2R)-2-[[5-(5-hydroxy-1-tetrahydropyran-2-yl-indazol-3-yl)-3-pyridyl]oxy]propoxy]-1-methyl-propyl]methane sulfonate (270 mg, 0.52 mmol) in dry DMF (14 mL). The reaction mixture was stirred at 60° C. for 1 h. The reaction was cooled to RT and diluted with water and ethyl acetate. The layers were separated. The aqueous layer was extracted with ethyl acetate and the combined organic layers were dried over anhydrous sodium sulfate, filtered and evaporated under reduced pressure. The residue was purified by silica gel column chromatography using cyclohexane/ethyl acetate 100/0 to 70/30 as eluent to afford (8R,13S)-8,13-dimethyl-19-(oxan-2-yl)-7,10,14-trioxa-4,19,20-triazatetracyclo[13.5.2.1^{2,6}.0^{18,21}]tricosa-1(20),2(23),3,5,15(22),16,18(21)-heptaene as a colorless oil.

LCMS method F: [M+H]⁺=424.4, t_{R}=2.85 min

### Preparation of Example 21: (8R,13S)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene

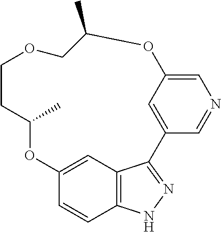 [SMILES: C[C@@H]1COCC[C@H](C)Oc2ccc3[nH]nc(c3c2)-c2cncc(c2)O1]

To a solution of (8R,13S)-8,13-dimethyl-19-(oxan-2-yl)-7,10,14-trioxa-4,19,20-triazatetra cyclo[13.5.2.1^{2,6}.0^{18,21}]tricosa-1(20),2(23),3,5,15(22),16,18(21)-heptaene (80 mg, 0.19 mmol) in methanol (0.6 mL) and water (0.1 mL) was added p-toluenesulfonic acid monohydrate (180 mg, 0.95 mmol). The reaction mixture was stirred at 65° C. for 96 h. The mixture was cooled to RT and diluted with a saturated aqeuous NaHCO₃ solution and ethyl acetate. The layers were separated. The aqueous layer was extracted with ethyl acetate and the combined organic layers were dried over anhydrous sodium sulfate, filtered and evaporated under reduced pressure. The residue was purified by silica gel column chromatography using dichloromethane/methanol 100/0 to 97/3 as eluent to afford (8R,13S)-8,13-dimethyl-7,10,14-trioxa-4,19,20-triazatetra cyclo[13.5.2.1^{2,6}.0^{18,21}]tricosa-1(20),2(23),3,5,15(22),16,18(21)-heptaene as a solid.

LCMS method F: [M+H]⁺=340.3, t_{R}=2.13 min

LCMS method G: [M+H]⁺=340.3, t_{R}=2.48 min

¹H NMR (400 MHz, CDCl₃) δ 10.34 (1H, s), 9.02 (1H, s), 8.54 (1H, s), 8.32 (1H, d, J=2.6 Hz), 7.97 (1H, d, J=2.3 Hz), 7.46 (1H, d, J=8.9 Hz), 7.10 (1H, dd, J=8.9 Hz, J=2.3 Hz), 4.73-4.65 (2H, m), 3.93-3.82 (2H, m), 3.61 (1H, dd, J=1.4, 10.5 Hz), 3.54-3.47 (1H, m), 2.70-2.63 (1H, m), 1.65-1.58 (1H, m), 1.52 (3H, d, J=6.0 Hz), 1.48 (3H, d, J=6.5 Hz) ppm.

### Example 22: (8S,13R)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene

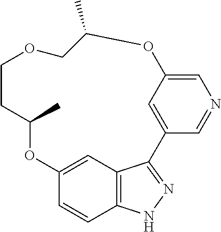 [SMILES: C[C@@H]1CCOC[C@H](C)Oc2cncc(c2)-c2n[nH]c3ccc(cc23)O1]

Example 22 is prepared according to the synthesis route described in general Scheme D following the same synthesis procedures as for Example 20.

### Preparation of Intermediate 142: [(1S)-3-[(2S)-2-[[5-(5-hydroxy-1-tetrahydropyran-2-yl-indazol-3-yl)-3-pyridyl]oxy]propoxy]-1-methyl-propyl] methanesulfonate

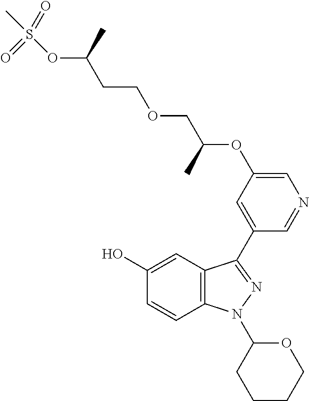 [SMILES: C[C@@H](COCC[C@H](C)OS(C)(=O)=O)Oc1cncc(-c2nn(C3CCCCO3)c3ccc(O)cc23)c1]

To a solution of [(1S)-3-[(2S)-2-[[5-[5-[tert-butyl(dimethyl)silyl]oxy-1-tetrahydropyran-2-yl-indazol-3-yl]-3-pyridyl]oxy]propoxy]-1-methyl-propyl]methanesulfonate (671 mg, 1.06 mmol) in THF (5.5 mL) at 0° C. was added TBAF (1M solution in THF) (1.06 mL, 1.06 mmol). The reaction mixture was allowed to warm up to RT and was stirred at RT for 2 h. The solvent was evaporated under reduced pressure. The residue was purified by silica gel column chromatography using dichloromethane/methanol 100/0 to 95/5 as eluent. The desired fractions were combined and the solvent was removed under reduced pressure to afford [(1S)-3-[(2S)-2-[[5-(5-hydroxy-1-tetrahydropyran-2-yl-indazol-3-yl)-3-pyridyl]oxy]propoxy]-1-methyl-propyl]methanesulfonate as a yellow oil.

LCMS method F intermediate 142: [M+H]⁺=520.4, t_{R}=2.26 min

### Preparation of Intermediate 143: (8S,13R)-8,13-dimethyl-19-(oxan-2-yl)-7,10,14-trioxa-4,19,20-triazatetracyclo[13.5.2.1^{2,6}.0^{18,21}]tricosa-1(20),2(23),3, 5,15(22),16, 18(21)-heptaene

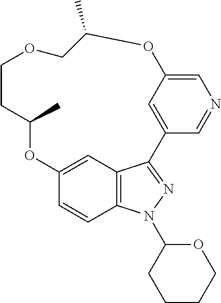 [SMILES: C[C@@H]1CCOC[C@H](C)Oc2cncc(c2)-c2nn(C3CCCCO3)c3ccc(cc23)O1]

To a solution of [[(1S)-3-[(2S)-2-[[5-(5-hydroxy-1-tetrahydropyran-2-yl-indazol-3-yl)-3-pyridyl]oxy]propoxy]-1-methyl-propyl]methanesulfonate (435 mg, 0.73 mmol) in methanol (14 mL) and THF (14 mL) was added dropwise 1M aqueous NaOH solution (3.65 mL, 3.65 mmol). The reaction mixture was stirred at 50° C. for 48 h. The reaction was cooled to RT and the solvents were removed under reduced pressure. The residue was diluted with water and ethyl acetate. The aqueous layer was extracted with ethyl acetate and the combined organic layers were dried over anhydrous sodium sulfate, filtered and the solvent was evaporated under reduced pressure. The residue was purified by silica gel column chromatography using cyclohexane/ethyl acetate 100/0 to 50/50 as eluent to afford (8S,13R)-8,13-dimethyl-19-(oxan-2-yl)-7,10,14-trioxa-4,19,20-triazatetracyclo[13.5.2.1^{2,6}.0^{18,21}]tricosa-1(20),2(23),3,5,15(22), 16,18(21)-heptaene as a white foam.

LCMS method F: [M+H]⁺=424.4, t_{R}=2.84 min

### Preparation of Example 22: (8S,13R)-8,13-dimethyl-7,10,14-trioxa-4,19,20-triaza tetracyclo[13.5.2.1^{2,6}.0^{18,21}]tricosa-1 (20),2(23),3,5,15(22) 16,18(21)-heptaene

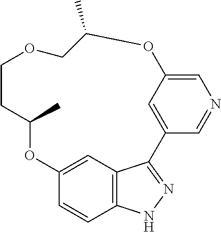 [SMILES: C[C@@H]1CCOC[C@H](C)Oc2cncc(c2)-c2n[nH]c3ccc(cc23)O1]

To a solution of (8S,13R)-8,13-dimethyl-19-(oxan-2-yl)-7,10,14-trioxa-4,19,20-triazatetra cyclo[13.5.2.1^{2,6}.0^{18,21}]tricosa-1(20),2(23),3,5,15(22),16,18(21)-heptaene (164 mg, 0.38 mmol) in methanol (1.2 mL) and water (0.2 mL) was added p-toluenesulfonic acid monohydrate (368 mg, 1.93 mmol). The reaction mixture was stirred at 65° C. for 96 h. The mixture was cooled to RT and diluted with a saturated aqueous NaHCO₃ solution and ethyl acetate. The layers were separated. The aqueous layer was extracted with ethyl acetate and the combined organic layers were dried over anhydrous sodium sulfate, filtered and evaporated under reduced pressure. The residue was purified by silica gel column chromatography using dichloromethane/methanol 100/0 to 97/3 as eluent to afford (8S,13R)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene as a white solid. NMR analysis showed presence of another diastereoisomer (S,S). A recrystallization was performed from acetonitrile. The mixture was filtered. A second recrystallization was performed from acetonitrile. The mixture was filtered to give (8S,13R)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene as a solid.

LCMS method F: [M+H]⁺=340.3, t_{R}=2.13 min

LCMS method G: [M+H]⁺=340.3, t_{R}=2.48 min

¹H NMR (400 MHz, CDCl₃) δ 10.53 (1H, s), 9.05 (1H, d, J=1.5 Hz), 8.59-8.56 (1H, m), 8.31 (1H, d, J=2.7 Hz), 7.96 (1H, d, J=2.1 Hz), 7.49-7.46 (1H, m), 7.10 (1H, dd, J=2.3, 8.9 Hz), 4.73-4.67 (2H, m), 3.94-3.82 (2H, m), 3.62 (1H, dd, J=1.7, 10.4 Hz), 3.54-3.47 (1H, m), 2.71-2.63 (1H, m), 1.65-1.58 (1H, m), 1.52 (3H, d, J=6.0 Hz), 1.48 (3H, d, J=6.7 Hz) ppm.

### Example 23: (8R,13R)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene

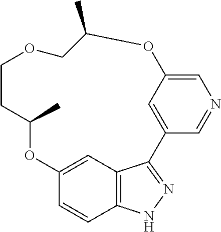 [SMILES: C[C@@H]1CCOC[C@@H](C)Oc2cncc(c2)-c2n[nH]c3ccc(cc23)O1]

Example 23 is prepared according to the synthesis route described in general Scheme D following the same synthesis procedures as for Example 20.

### Preparation of Intermediate 144: (8R,13R)-8,13-dimethyl-19-(oxan-2-yl)-7,10,14-trioxa-4,19,20-triazatetracyclo[13.5.2.1^{2,6}.0^{18,21}]tricosa-1(20),2(23),3,5,15(22),16, 18(21)-heptaene

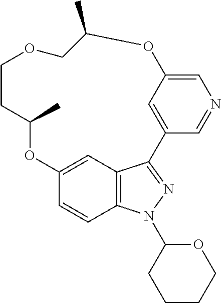 [SMILES: C[C@@H]1CCOC[C@@H](C)Oc2cncc(c2)-c2nn(C3CCCCO3)c3ccc(cc23)O1]

To a solution of cesium carbonate (556 mg, 1.71 mmol) in dry DMF (18 mL) heated at 60° C. was added dropwise under argon atmosphere a solution of [(1S)-3-[(2R)-2-[[5-(5-hydroxy-1-tetrahydropyran-2-yl-indazol-3-yl)-3-pyridyl]oxy]propoxy]-1-methyl-propyl]methane sulfonate (355 mg, 0.68 mmol) in dry DMF (18 mL). The reaction mixture was stirred at 60° C. for 1 h. The reaction was cooled to RT and diluted with water and ethyl acetate. The layers were separated. The aqueous layer was extracted with ethyl acetate and the combined organic layers were dried over anhydrous sodium sulfate, filtered and evaporated under reduced pressure. The residue was purified by silica gel column chromatography using cyclohexane/ethyl acetate 100/0 to 70/30 as eluent to afford (8R,13R)-8,13-dimethyl-19-(oxan-2-yl)-7,10,14-trioxa-4,19,20-triazatetracyclo[13.5.2.1^{2,6}.0^{18,21}]tricosa-1(20),2(23),3,5,15(22),16,18(21)-heptaene as a white solid.

LCMS method F: [M+H]⁺=424.4, t_{R}=2.87 min

### Preparation of Example 23: (8R,13R)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene

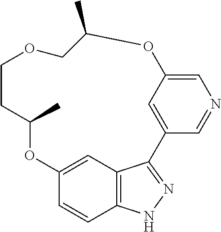 [SMILES: C[C@@H]1CCOC[C@@H](C)Oc2cncc(c2)-c2n[nH]c3ccc(cc23)O1]

To a solution of (8R,13R)-8,13-dimethyl-19-(oxan-2-yl)-7,10,14-trioxa-4,19,20-triazatetra cyclo[13.5.2.1^{2,6}.0^{18,21}]tricosa-1(20),2(23),3,5,15(22),16,18(21)-heptaene (135 mg, 0.32 mmol) in methanol (1 mL) and water (0.2 mL) was added p-toluenesulfonic acid monohydrate (304 mg, 1.60 mmol). The reaction mixture was stirred at 65° C. for 1 h. The mixture was cooled to RT and diluted with a saturated aqueous NaHCO₃ solution and ethyl acetate. The layers were separated. The aqueous layer was extracted with ethyl acetate and the combined organic layers were dried over anhydrous sodium sulfate, filtered and evaporated under reduced pressure. The residue was purified by silica gel column chromatography using dichloromethane/methanol 100/0 to 97/3 as eluent to afford (8R,13R)-8,13-dimethyl-7,10,14-trioxa-4,19,20-triazatetra cyclo[13.5.2.1^{2,6}.0^{18,21}]tricosa-1(20),2(23),3,5,15(22),16,18(21)-heptaene as a solid.

LCMS method F: [M+H]⁺=340.3, t_{R}=2.15 min

LCMS method G: [M+H]⁺=340.3, t_{R}=2.47 min

¹H NMR (400 MHz, d6-DMSO) δ 13.2 (1H, s), 8.64 (1H, d, J=1.5 Hz), 8.31-8.30 (1H, m), 8.21 (1H, d, J=2.7 Hz), 7.65 (1H, d, J=1.9 Hz), 7.52 (1H, d, J=9.0 Hz), 7.00 (1H, dd, J=2.2, 9.0 Hz), 4.65-4.61 (1H, m), 4.54-4.46 (1H, m), 3.93 (1H, dd, J=6.0, 12.1 Hz), 3.72-3.68 (1H, m), 3.64-3.55 (2H, m), 2.38-2.30 (1H, m), 1.68-1.60 (1H, m), 1.39 (3H, d, J=6.1 Hz), 1.37 (3H, d, J=6.8 Hz) ppm.

### Example 24: (13S)-13-methyl-8,11,14-trioxa-4,5,19,20-tetraazatetracyclo[13.5.2.1^{2,5}.0^{18,21}]tricosa-1(20),2(23),3,15(22),16,18(21)-hexaene

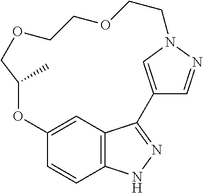 [SMILES: C[C@H]1COCCOCCn2cc(cn2)-c2n[nH]c3ccc(cc23)O1]

Example 24 is prepared according to the synthesis route described in general Scheme D.

### Preparation of Intermediate 145: 2-(2-benzyloxyethoxy)ethyl methanesulfonate

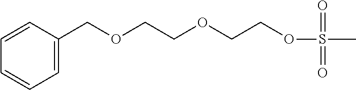 [SMILES: CS(=O)(=O)OCCOCCOCc1ccccc1]

To a suspension 2-(2-benzyloxyethoxy)ethanol (2 g, 10.20 mmol) in dichloromethane (40 mL) at 0° C. was added triethylamine (2.12 mL, 15.30 mmol) and methanesulfonyl chloride (0.86 mL, 11.22 mmol). The reaction mixture was stirred at RT for 1 h. The mixture was diluted with water. The layers were separated. The aqueous layer was extracted with dichloromethane and the combined organic layer were washed with a saturated aqueous NH₄Cl solution, water and brine. The organic layer was dried over anhydrous sodium sulfate, filtered and the solvent was evaporated under reduced pressure to afford 2-(2-benzyloxyethoxy)ethylmethanesulfonate as a colorless oil which was used in the next step without any further purification.

LCMS method F: [M+H]⁺=275.2, t_{R}=2.12 min

### Preparation of Intermediate 146: 1-[2-(2-benzyloxyethoxy)ethyl]-4-bromo-pyrazole

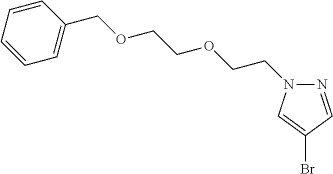 [SMILES: Brc1cnn(CCOCCOCc2ccccc2)c1]

A suspension of 4-bromo-1H-pyrazole (1.308 g, 8.96 mmol), 2-(2-benzyloxyethoxy)ethyl methanesulfonate (2.702 g, 9.86 mmol), and cesium carbonate (3.783 g, 11.64 mmol) in acetonitrile (36 mL) was stirred at 85° C. for 16 h. The reaction was quenched by water and the resulting solution was extracted with ethyl acetate. The combined organic layers were dried over anhydrous magnesium sulfate, filtered, and the solvent was removed under reduced pressure to afford 1-[2-(2-benzyloxyethoxy)ethyl]-4-bromo-pyrazole as a colorless oil which was used in the next step without further purification.

LCMS method F: [M+H]⁺=325.1, t_{R}=2.54 min

### Preparation of Intermediate 147: 2-[2-(4-bromopyrazol-1-yl)ethoxy]ethanol

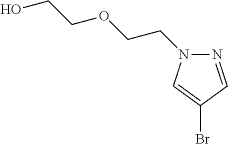 [SMILES: OCCOCCn1cc(Br)cn1]

To a solution of 1-[2-(2-benzyloxyethoxy)ethyl]-4-bromo-pyrazole (2.912 g, 8.96 mmol) in ethanol (60 mL) was added hydrochloric acid (37% aqueous solution) (45 mL). The reaction mixture was stirred at 80° C. for 24 h. The reaction mixture was cooled down to RT and concentrated under reduced pressure. The residue was dissolved in a saturated aqueous NaHCO₃ solution and extracted with ethyl acetate. The combined organic layers were washed with water, brine, dried over anhydrous magnesium sulfate, filtered and the solvent was removed under reduced pressure to afford 2-[2-(4-bromopyrazol-1-yl)ethoxy]ethanol as a colorless oil.

LCMS method F: [M+H]⁺=235.1-237.1, t_{R}=1.40 min

### Preparation of Intermediate 148: 1-[2-[2-[(2R)-2-benzyloxypropoxy]ethoxy]ethyl]-4-bromo-pyrazole

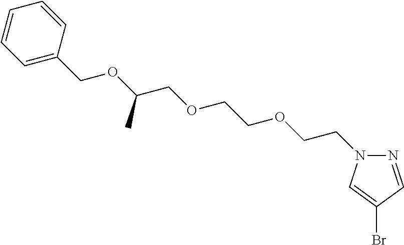 [SMILES: C[C@H](COCCOCCn1cc(Br)cn1)OCc1ccccc1]

To a stirred solution of 2-[2-(4-bromopyrazol-1-yl)ethoxy]ethanol (1.827 g, 7.77 mmol) in DMF (45 mL) was added portionwise sodium hydride (60% dispersion in mineral oil) (621 mg, 9.32 mmol). The reaction mixture was stirred at RT for 30 min and [(2R)-2-benzyloxypropyl] 4-methylbenzenesulfonate (intermediate 88) (2.736 g, 8.55 mmol) was added. The reaction mixture was stirred at 55° C. for 3 h. The solvent was evaporated under reduced pressure, diluted with water and extracted with ethyl acetate. The combined organic layers were washed with water, dried over anhydrous magnesium sulfate, filtered and the solvent was removed under reduced pressure. The residue was purified by silica gel column chromatography using dichloromethane/methanol 100/0 to 97/3 as eluent to afford 1-[2-[2-[(2R)-2-benzyloxypropoxy]ethoxy]ethyl]-4-bromo-pyrazole as a yellow oil.

LCMS method F: [M+H]⁺=383.3-385.3, t_{R}=2.70 min

### Preparation of Intermediate 149: [3-[1-[2-[2-[(2R)-2-benzyloxypropoxy]ethoxy] ethyl]pyrazol-4-yl]-1-tetrahydropyran-2-yl-indazol-5-yl]oxy-tert-butyl-dimethyl-silane

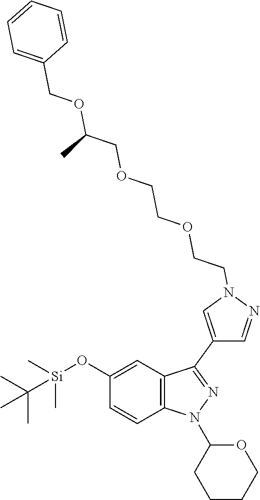 [SMILES: C[C@H](COCCOCCn1cc(-c2nn(C3CCCCO3)c3ccc(O[Si](C)(C)C(C)(C)C)cc23)cn1)OCc1ccccc1]

To solution of 1-[2-[2-[(2R)-2-benzyloxypropoxy]ethoxy]ethyl]-4-bromo-pyrazole (2.103 g, 5.49 mmol), tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (3.013 g, 6.58 mmol) and potassium phosphate tribasic (3.491 g, 16.47 mmol) in dioxane (50 mL) and water (2 mL) were added tetrakis(triphenylphosphine)palladium(0) (312 mg, 0.27 mmol) and 2-dicyclohexylphosphino-2′,4′,6′-triisopropylbiphenyl (261 mg, 0.55 mmol). The reaction mixture was stirred at 150° C. for 3 h. The reaction mixture was filtered through a pad of celite and was washed with ethyl acetate. The filtrate was diluted with water and extracted with ethyl acetate. The combined organic layers were washed with water, brine, dried over anhydrous magnesium sulfate, filtered and the solvent was removed under reduced pressure. The residue was purified by silica gel column chromatography using dichloromethane/methanol 100/0 to 98/2 as eluent to afford [3-[1-[2-[2-[(2R)-2-benzyloxypropoxy]ethoxy]ethyl]pyrazol-4-yl]-1-tetrahydropyran-2-yl-indazol-5-yl]oxy-tert-butyl-dimethyl-silane as a yellow oil.

LCMS method F: [M+H]⁺=635.5, t_{R}=3.76 min

### Preparation of Intermediate 150: (2R)-1-[2-[2-[4-[5-[tert-butyl(dimethyl)silyl]oxy-1-tetrahydropyran-2-yl-indazol-3-yl]pyrazol-1-yl]ethoxy]ethoxy]propan-2-ol

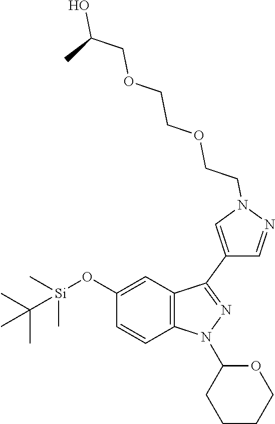 [SMILES: C[C@@H](O)COCCOCCn1cc(-c2nn(C3CCCCO3)c3ccc(O[Si](C)(C)C(C)(C)C)cc23)cn1]

To a solution of [3-[1-[2-[2-[(2R)-2-benzyloxypropoxy]ethoxy]ethyl]pyrazol-4-yl]-1-tetrahydropyran-2-yl-indazol-5-yl]oxy-tert-butyl-dimethyl-silane (1.618 g, 2.55 mmol) in ethanol (37 mL) at RT was added palladium hydroxide on carbon (200 mg). The reaction mixture was stirred under hydrogen atmosphere at RT overnight. The reaction mixture filtered and the filtrate was concentrated under reduced pressure. The residue was purified by silica gel column chromatography using cyclohexane/ethyl acetate 100/0 to 20/80 as eluent to afford (2R)-1-[2-[2-[4-[5-[tert-butyl(dimethyl)silyl]oxy-1-tetrahydropyran-2-yl-indazol-3-yl] pyrazol-1-yl]ethoxy]ethoxy]propan-2-ol as a colorless oil.

LCMS method F: [M+H]⁺=545.4, t_{R}=3.21 min

### Preparation of Intermediate 151: [3-[1-[2-[2-[(2R)-2-benzyloxypropoxy]ethoxy] ethyl]pyrazol-4-yl]-1-tetrahydropyran-2-yl-indazol-5-yl]oxy-tert-butyl-dimethyl-silane

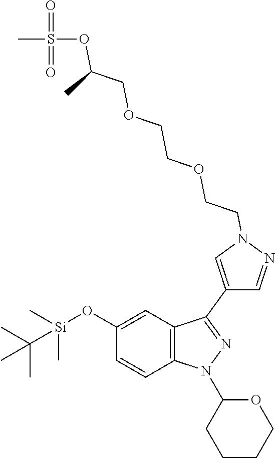 [SMILES: C[C@H](COCCOCCn1cc(-c2nn(C3CCCCO3)c3ccc(O[Si](C)(C)C(C)(C)C)cc23)cn1)OS(C)(=O)=O]

To a solution of (2R)-1-[2-[2-[4-[5-[tert-butyl(dimethyl)silyl]oxy-1-tetrahydropyran-2-yl-indazol-3-yl]pyrazol-1-yl]ethoxy]ethoxy]propan-2-ol (858 mg, 1.58 mmol) and triethylamine (440 μL, 3.16 mmol) in dichloromethane (20 mL) at 0° C. was added dropwise methanesulfonyl chloride (360 μL, 4.73 mmol). The reaction mixture was stirred RT for 2 h. The mixture was diluted with water and extracted with dichloromethane. The combined organic layers were washed with brine, dried over anhydrous magnesium sulfate and the solvent was removed under reduced pressure to afford [(1R)-2-[2-[2-[4-[5-[tert-butyl(dimethyl)silyl]oxy-1-tetrahydro pyran-2-yl-indazol-3-yl]pyrazol-1-yl]ethoxy]ethoxy]-1-methyl-ethyl] methanesulfonate as a colorless oil which was used in the next step without further purification.

LCMS method K: [M+H]⁺=623.5, t_{R}=3.36 min

### Preparation of Intermediate 152: (13S)-13-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene

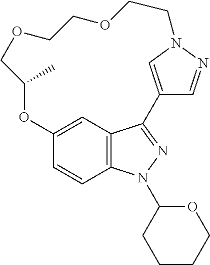 [SMILES: C[C@H]1COCCOCCn2cc(cn2)-c2nn(C3CCCCO3)c3ccc(cc23)O1]

To a suspension of cesium carbonate (1.405 g, 4.32 mmol) in anhydrous DMF (75 mL) at 60° C. was added dropwise [(1R)-2-[2-[2-[4-[5-[tert-butyl(dimethyl)silyl]oxy-1-tetrahydropyran-2-yl-indazol-3-yl]pyrazol-1-yl]ethoxy]ethoxy]-1-methyl-ethyl]methanesulfonate (900 mg, 1.44 mmol) in DMF (35 mL). The reaction mixture was stirred at 60° C. for 16 h. The reaction mixture filtered over a pad of celite and rinsed with ethyl acetate. The filtrate was concentrated under reduced pressure. The residue was purified by silica gel column chromatography using dichloromethane/methanol 100/0 to 95/5 as eluent. The desired fractions were combined and the solvent was removed under reduced pressure to afford (13S)-13-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16, 18(21)-hexaene as an orange solid.

LCMS method F: [M+H]⁺=413.4, t_{R}=2.54 min

### Preparation of Example 24: (13S)-13-methyl-8,11,14-trioxa-4,5,19,20-tetraazatetra cyclo[13.5.2.1^{2,5}.0^{18,21}]tricosa-1(20),2(23),3,15(22),16,18(21)-hexaene

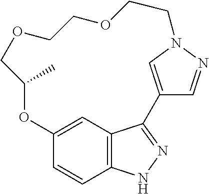 [SMILES: C[C@H]1COCCOCCn2cc(cn2)-c2n[nH]c3ccc(cc23)O1]

To a solution of (13S)-13-methyl-19-(oxan-2-yl)-8,11,14-trioxa-4,5,19,20-tetraaza tetracyclo[13.5.2.1^{2,5}.0^{18,21}]tricosa-1(20),2(23),3,15(22),16,18(21)-hexaene (348 mg, 0.84 mmol) in dichloromethane (5 mL) at RT was added TFA (1.25 mL, 16.8 mmol). The reaction mixture was stirred at RT for 16 h. The reaction mixture was concentrated under reduced pressure, diluted with a saturated sodium bicarbonate solution and extracted with ethyl acetate. The combined organic layers were dried over anhydrous sodium sulfate, filtered and evaporated under reduced pressure The resulting solid was triturated in diethyl ether and filtered. The collecting solid was diluted in dichloromethane, evaporated under reduced pressure and triturated in diethyl ether, then collected by filtration. This procedure was repeated until the obtained solid was completely pure to afford (13S)-13-methyl-8,11,14-trioxa-4,5,19,20-tetraazatetracyclo[13.5.2.1^{2,5}.0^{18,21}]tricosa-1(20),2(23),3,15(22),16,18(21)-hexaene as a solid.

LCMS method F: [M+H]⁺=329.3, t_{R}=1.96 min

LCMS method H: [M+H]⁺=329.3, t_{R}=3.01 min

¹H NMR (400 MHz, d6-DMSO) δ 12.74 (1H, s), 8.46 (1H, s), 7.82-7.79 (2H, m), 7.38 (1H, d, J=8.8 Hz), 6.98 (1H, dd, J=1.6, 8.8 Hz), 4.41-4.33 (3H, m), 3.90-3.87 (1H, m), 3.80-3.61 (6H, m), 3.53 (1H, dd, J=2.2, 10.2 Hz), 1.35 (3H, d, J=6.5 Hz) ppm.

### Example 25: (6R,12S)-6,12-dimethyl-9,13-dioxa-4,5,18,19-tetraazatetracyclo[12.5.2.1^{2,5}.0^{17,20}]docosa-1(19),2(22),3,14(21),15,17(20)-hexaene

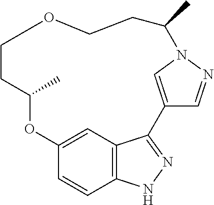 [SMILES: C[C@@H]1CCOCC[C@H](C)Oc2ccc3[nH]nc(c3c2)-c2cnn1c2]

Example 25 is prepared according to the synthesis route described in general Scheme D.

### Preparation of Intermediate 153: (2S)-4-[(tert-butyldiphenylsilyl)oxy]butan-2-ol

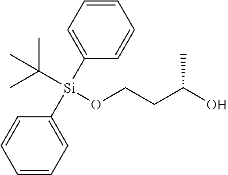 [SMILES: C[C@H](O)CCO[Si](c1ccccc1)(c1ccccc1)C(C)(C)C]

To a solution of (3S)-butane-1,3-diol (5.05 g, 56.04 mmol) in THF (277 mL) was added imidazole (7.63 g, 112.07 mmol) and tert-butyldiphenylchlorosilane (14.572 mL, 56.04 mmol). The reaction mixture was stirred at RT for 4 h. Water and ethyl acetate were added and the layers were separated. The aqueous layer was extracted with ethyl acetate and the combined organic layers were dried over anhydrous sodium sulfate, filtered and evaporated under reduced pressure. The residue was purified by silica gel column chromatography using cyclohexane/ethyl acetate 100/0 to 90/10 as eluent to afford (2S)-4-[(tert-butyldiphenylsilyl)oxy]butan-2-ol as a colorless oil.

LCMS method F: [M+H]⁺=329.3, t_{R}=3.36 min

### Preparation of Intermediate 154: [(1S)-3-[tert-butyl(diphenyl)silyl]oxy-1-methyl-propyl] methanesulfonate

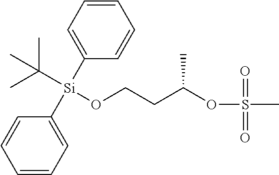 [SMILES: C[C@@H](CCO[Si](c1ccccc1)(c1ccccc1)C(C)(C)C)OS(C)(=O)=O]

To a solution of (2S)-4-[tert-butyl(diphenyl)silyl]oxybutan-2-ol (1.515 g, 4.619 mmol) and triethylamine (1.288 mL, 9.238 mmol) in dry dichloromethane (32 mL) at 0° C. was added dropwise methanesulfonyl chloride (0.465 mL, 6.005 mmol). The reaction mixture was stirred at RT for 20 h. The reaction mixture was quenched by water. The aqueous layer was extracted with dichloromethane. The combined organic layers were washed with water and brine, dried over anhydrous sodium sulfate, filtered and evaporated under reduced pressure to give [(1S)-3-[tert-butyl(diphenyl)silyl]oxy-1-methyl-propyl]methanesulfonate as an orange oil.

LCMS method L: [M+H]⁺=407.4, t_{R}=3.46 min

### Preparation of Intermediate 155: [(3R)-3-(4-bromopyrazol-1-yl)butoxy]-tert-butyl-diphenyl-silane

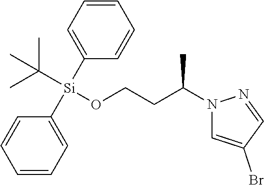 [SMILES: C[C@H](CCO[Si](c1ccccc1)(c1ccccc1)C(C)(C)C)n1cc(Br)cn1]

To a suspension of 4-bromo-1H-pyrazole (562 mg, 3.849 mmol) and cesium carbonate (1.881 g, 5.774 mmol) in dry acetonitrile (25 mL) at RT was added [(1S)-3-[tert-butyl(diphenyl) silyl]oxy-1-methyl-propyl] methanesulfonate (1.875 g, 4.619 mmol) in dry acetonitrile (5 mL). The reaction mixture was stirred at 85° C. for 16 h. The reaction mixture was filtered and ethyl acetate and water were added. The aqueous layer was extracted with ethyl acetate. The combined organic layers were washed with water and brine, dried over sodium sulfate, filtered and the solvent was removed under reduced pressure. The residue was purified by silica gel column chromatography using cyclohexane/ethyl acetate 100/0 to 80/20 as eluent to afford [(3R)-3-(4-bromopyrazol-1-yl)butoxy]-tert-butyl-diphenyl-silane as a colorless oil.

LCMS method F: [M+H]⁺=457.1-459.1, t_{R}=3.84 min

### Preparation of Intermediate 156: (3R)-3-(4-bromopyrazol-1-yl)butan-1-ol

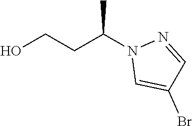

To a solution of [(3R)-3-(4-bromopyrazol-1-yl)butoxy]-tert-butyl-diphenyl-silane (1.675 g, 3.673 mmol) in dry THF (8 mL) was added TBAF (1M solution in THF) (4.04 mL, 4.04 mmol). The reaction mixture was stirred at RT for 16 h. Ice water was added and the reaction mixture was stirred for 20 min. The aqueous phase was extracted with ethyl acetate. The combined organic layers were washed with brine dried over anhydrous sodium sulfate, filtered and evaporated under reduced pressure. The residue was purified by silica gel column chromatography using cyclohexane/ethyl acetate 100/0 to 80/20 as eluent to afford (3R)-3-(4-bromopyrazol-1-yl)butan-1-ol as a colorless oil.

LCMS method F: [M+H]⁺=219.1-221.1, t_{R}=1.77 min

### Preparation of Intermediate 157: 1-methoxy-4-[[(1R)-1-methyl-3-trityloxy-propoxy] methyl]benzene

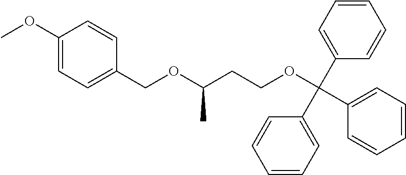 [SMILES: COc1ccc(CO[C@H](C)CCOC(c2ccccc2)(c2ccccc2)c2ccccc2)cc1]

To solution of (2R)-4-trityloxybutan-2-ol (intermediate 126) (5.89 g, 17.73 mmol) in dry DMF (30 mL) at 0° C. was added dropwise sodium hydride (60% dispersion in oil) (1.064 g, 26.60 mmol). The reaction mixture was stirred at 0° C. for 20 min. A solution of 4-methoxybenzyl chloride (5 g, 31.92 mmol) in dry DMF (4 mL) was added dropwise and the suspension was stirred at RT for 48 h. The reaction mixture was quenched by water and the aqueous layer was extracted with ethyl acetate. The combined organic layers were washed with water and brine, dried over anhydrous sodium sulfate, filtered and evaporated under reduced pressure. The residue was purified by silica gel column chromatography using cyclohexane/ethyl acetate 100/0 to 90/10 as eluent to afford 1-methoxy-4-[[(1R)-1-methyl-3-trityloxy-propoxy]methyl]benzene as a colorless oil.

LCMS method F: [M+Na]⁺=475.2, t_{R}=3.69 min

### Preparation of Intermediate 158: (3R)-3-[(4-methoxyphenyl)methoxy]butan-1-ol

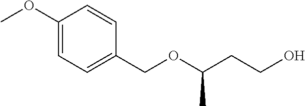

A solution of 1-methoxy-4-[[(1R)-1-methyl-3-trityloxy-propoxy]methyl]benzene (7.407 g, 16.38 mmol) in a mixture of water (19.5 mL), acetic acid (26 mL) and methanol (19.5 ml) was heated to 50° C. for 24 h. The reaction mixture was filtered, washed with water and cyclohexane. The filtrate was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 70/30 as eluent to afford (3R)-3-[(4-methoxyphenyl)methoxy]butan-1-ol as a colorless liquid.

LCMS method F: [M+Na]⁺=233.1, t_{R}=2.02 min

### Preparation of Intermediate 159: [(3R)-3-[(4-methoxyphenyl)methoxy]butyl] methanesulfonate

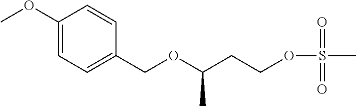

To a solution of (3R)-3-[(4-methoxyphenyl)methoxy]butan-1-ol (950 mg, 4.52 mmol) and triethylamine (1.26 mL, 9.04 mmol) in dry dichloromethane (31 mL) at 0° C. was added dropwise methanesulfonyl chloride (455 μL, 5.88 mmol). The reaction mixture was stirred at RT for 3 h. The reaction mixture was quenched by water and the aqueous layer was extracted with dichloromethane. The combined organic layers were washed with water then brine, dried over anhydrous sodium sulfate, filtered and evaporated under reduced pressure to give [(3R)-3-[(4-methoxyphenyl)methoxy]butyl] methanesulfonate as a pale yellow oil.

LCMS method F: [M+Na]⁺=311.1, t_{R}=2.43 min

### Preparation of Intermediate 160: 4-bromo-1-[(1R)-3-[(3R)-3-[(4-methoxyphenyl) methoxy]butoxy]-1-methyl-propyl]pyrazole

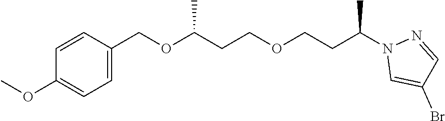 [SMILES: COc1ccc(CO[C@H](C)CCOCC[C@@H](C)n2cc(Br)cn2)cc1]

To a solution of (3R)-3-(4-bromopyrazol-1-yl)butan-1-ol (706 mg, 3.22 mmol) in dry DMF (25 mL) at 0° C. was added portionwise sodium hydride (60% dispersion in oil) (193 mg, 4.83 mmol). The reaction mixture was stirred at 0° C. for 1 h. A solution of [(3R)-3-[(4-methoxyphenyl)methoxy]butyl] methanesulfonate (1.26 g, 4.38 mmol) in dry DMF (5 mL) was added dropwise at 0° C. The reaction mixture was stirred at RT for 72 h. Water was added and the aqueous layer was extracted with ethyl acetate. The combined organic layers were washed with water and brine, dried over anhydrous sodium sulfate, filtered and evaporated under reduced pressure. The residue was purified by silica gel column chromatography using cyclohexane/(ethyl acetate/ethanol (3-1)), 100/0 to 80/20 as eluent to afford 4-bromo-1-[(1R)-3-[(3R)-3-[(4-methoxyphenyl)methoxy]butoxy]-1-methyl-propyl]pyrazole as a pale yellow oil.

LCMS method L: [M+H]⁺=411.3413.3, t_{R}=3.02 min

### Preparation of Intermediate 161: tert-butyl-[3-[1-[(1R)-3-[(3R)-3-[(4-methoxy phenyl)methoxy]butoxy]-1-methyl-propyl]pyrazol-4-yl]-1-tetrahydropyran-2-yl-indazol-5-yl]oxy-dimethyl-silane

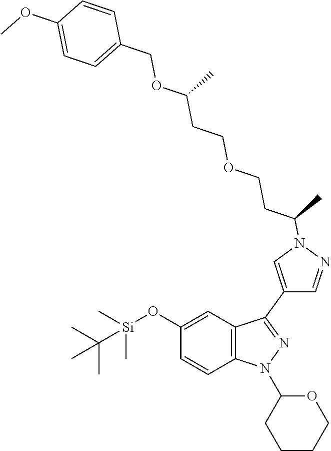 [SMILES: COc1ccc(CO[C@H](C)CCOCC[C@@H](C)n2cc(-c3nn(C4CCCCO4)c4ccc(O[Si](C)(C)C(C)(C)C)cc34)cn2)cc1]

To a suspension of 4-bromo-1-[(1R)-3-[(3R)-3-[(4-methoxyphenyl)methoxy]butoxy]-1-methyl-propyl]pyrazole (1.069 g, 2.60 mmol), tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (1.549 g, 3.38 mmol), potassium phosphate tribasic (1.657 g, 7.80 mmol) and XPhos (124 mg, 0.26 mmol) in dioxane (22.5 mL) and water (7.5 mL) was added tetrakis(triphenylphosphine) palladium(0) (150 mg, 0.130 mmol). The reaction mixture was stirred at 90° C. under microwave irradiations for 1 h. The reaction mixture was filtered and washed with ethyl acetate. Water was added and the aqueous layer was extracted with ethyl acetate. The combined organic layers were washed with brine, dried over anhydrous sodium sulfate and evaporated under reduced pressure. The residue was purified by silica gel column chromatography using cyclohexane/ethyl acetate 100/0 to 80/20 as eluent to afford tert-butyl-[3-[1-[(1R)-3-[(3R)-3-[(4-methoxyphenyl)methoxy]butoxy]-1-methyl-propyl]pyrazol-4-yl]-1-tetrahydropyran-2-yl-indazol-5-yl]oxy-dimethyl-silane as an orange pale/brown oil.

LCMS method F: [M+H]⁺=663.4, t_{R}=3.92 min

### Preparation of Intermediate 162: (2R)-4-[(3R)-3-[4-[5-[tert-butyl(dimethyl)silyl]oxy-1-tetrahydropyran-2-yl-indazol-3-yl]pyrazol-1-yl]butoxy]butan-2-ol

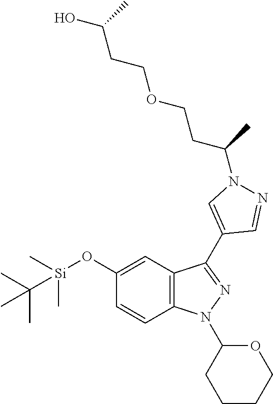 [SMILES: C[C@H](CCOCC[C@@H](C)O)n1cc(-c2nn(C3CCCCO3)c3ccc(O[Si](C)(C)C(C)(C)C)cc23)cn1]

To a solution of tert-butyl-[3-[1-[(1R)-3-[(3R)-3-[(4-methoxyphenyl)methoxy]butoxy]-1-methyl-propyl]pyrazol-4-yl]-1-tetrahydropyran-2-yl-indazol-5-yl]oxy-dimethyl-silane (933 mg, 1.409 mmol) in methanol (35 mL) at RT was added palladium on carbon (93 mg). The reaction mixture was stirred under hydrogen atmosphere at RT for 32 h. The reaction mixture was filtered and washed with methanol. The reaction mixture was concentrated under reduced pressure. The residue was purified by silica gel column chromatography using cyclohexane/(ethyl acetate/ethanol (3-1)) 100/0 to 60/40 as eluent to afford (2R)-4-[(3R)-3-[4-[5-[tert-butyl(dimethyl)silyl]oxy-1-tetrahydropyran-2-yl-indazol-3-yl]pyrazol-1-yl]butoxy] butan-2-ol as a pale yellow oil.

LCMS method F: [M+H]⁺=543.3, t_{R}=3.43 min

### Preparation of Intermediate 163: [(1R)-3-[(3R)-3-[4-[5-[tert-butyl(dimethyl)silyl] oxy-1-tetrahydropyran-2-yl-indazol-3-yl]pyrazol-1-yl]butoxy]-1-methyl-propyl] methanesulfonate

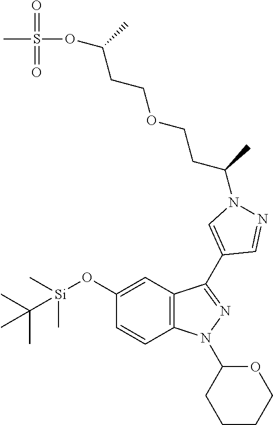 [SMILES: C[C@H](CCOCC[C@@H](C)n1cc(-c2nn(C3CCCCO3)c3ccc(O[Si](C)(C)C(C)(C)C)cc23)cn1)OS(C)(=O)=O]

To a solution of (2R)-4-[(3R)-3-[4-[5-[tert-butyl(dimethyl)silyl]oxy-1-tetrahydropyran-2-yl-indazol-3-yl]pyrazol-1-yl]butoxy]butan-2-ol (480 mg, 0.886 mmol) and triethylamine (247 μL, 1.772 mmol) in dry dichloromethane (19 mL) at 0° C. was added methanesulfonyl chloride (89 μL, 1.152 mmol). The reaction mixture was stirred at RT for 16 h. Additional methanesulfonyl chloride (14 μL, 0.177 mmol) was added at RT. The reaction mixture was stirred at RT for 4 h. The reaction was quenched with brine and extracted with dichloromethane. The combined organic layers were dried over anhydrous sodium sulfate, filtered and evaporated under reduced pressure to afford [(1R)-3-[(3R)-3-[4-[5-[tert-butyl(dimethyl)silyl]oxy-1-tetrahydropyran-2-yl-indazol-3-yl]pyrazol-1-yl]butoxy]-1-methyl-propyl]methanesulfonate as a colorless oil which was used in the next step without further purification.

LCMS method F: [M+H]⁺=621, t_{R}=5.49 min

### Preparation of Intermediate 164: (6R,12S)-6,12-dimethyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene

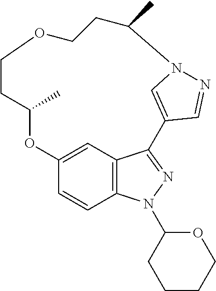 [SMILES: C[C@@H]1CCOCC[C@H](C)Oc2ccc3c(c2)c(nn3C2CCCCO2)-c2cnn1c2]

To a suspension of cesium carbonate (873 mg, 2.68 mmol) in dry DMF (200 mL) at 90° C. was added dropwise [(1R)-3-[(3R)-3-[4-[5-[tert-butyl(dimethyl)silyl]oxy-1-tetrahydropyran-2-yl-indazol-3-yl]pyrazol-1-yl]butoxy]-1-methyl-propyl]methanesulfonate (415 mg, 0.670 mmol) in dry DMF (200 mL). The reaction mixture was heated at 90° C. for 2 h and at RT for 16 h. The solvent was evaporated under reduced pressure then diluted with brine and extracted with ethyl acetate. The combined organic layers were dried over anhydrous sodium sulfate, filtered and evaporated under reduced pressure. The residue was purified by flash-column chromatography using dichloromethane/methanol 100/0 to 98/2 as eluent to afford (6R,12S)-6,12-dimethyl-18-(oxan-2-yl)-9,13-dioxa-4,5,18,19-tetraazatetracyclo[12.5.2.1^{2,5}.0^{17,20}]docosa-1(19),2(22),3, 14(21),15,17(20)-hexaene as a yellow oil.

LCMS method J: [M+H]⁺=411.3, t_{R}=4.20 min

### Preparation of Example 25: (6R,12S)-6,12-dimethyl-9,13-dioxa-4,5,18,19-tetraaza tetracyclo[12.5.2.1^{2,5}.0^{17,20}]docosa-1(19),2(22),3,14(21),15,17(20)-hexaene

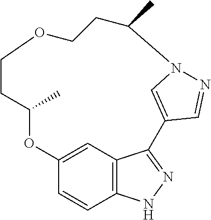 [SMILES: C[C@@H]1CCOCC[C@H](C)Oc2ccc3[nH]nc(c3c2)-c2cnn1c2]

To a solution of (6R,12S)-6,12-dimethyl-18-(oxan-2-yl)-9,13-dioxa-4,5,18,19-tetraazatetra cyclo[12.5.2.1^{2,5}.0^{17,20}]docosa-1(19),2(22),3,14(21),15,17(20)-hexaene (32 mg, 0.078 mmol) in methanol (4.6 mL) and water (0.6 mL) was added p-toluenesulfonic acid monohydrate (74 mg, 0.390 mmol). The reaction mixture was stirred at 65° C. for 4 h. The solvent was evaporated under reduced pressure and the residue was neutralized by addition of a saturated aqueous NaHCO₃ solution. The mixture was diluted with ethyl acetate and the aqueous layer was extracted with ethyl acetate. The combined organic layers were washed with water and brine, dried over sodium sulfate, filtered and evaporated under reduced pressure. The residue was purified by silica gel column chromatography using cyclohexane/(ethyl acetate/ethanol (3-1)) 100/0 to 60/40 as eluent. The resulting solid was triturated from hot diisopropyl ether, filtered and dried to afford (6R,12S)-6,12-dimethyl-9,13-dioxa-4,5,18,19-tetraazatetracyclo[12.5.2.1^{2,5}.0^{17,20}]docosa-1(19),2(22),3,14(21),15,17(20)-hexaene as a solid.

LCMS method F: [M+H]⁺=327.3, t_{R}=2.25 min

LCMS method H: [M+H]⁺=327.2, t_{R}=2.26 min

¹H NMR (400 MHz, MeOD) δ 8.79 (1H, s), 7.80 (1H, m), 7.58 (1H, m), 7.41-7.39 (1H, d), 7.02-7.00 (1H, dd, J=2.4, 9.0 Hz), 4.79-4.71 (1H, m), 4.62-4.55 (1H, m), 3.85-3.80 (1H, m), 3.77-3.71 (1H, m), 3.67-3.59 (2H, m), 2.62-2.56 (1H, m), 2.38-2.29 (1H, m), 2.28-2.20 (1H, m), 1.63 (3H, d), 1.61-1.55 (1H, m), 1.46 (3H, d, J=5.9 Hz) ppm.

### Example 26: (13S)-13-methyl-7,11,14-trioxa-5,19,20,23-tetraazatetracyclo[13.5.2.1^{2,6}.0^{18,21}]tricosa-1(20),2(23),3,5,15(22),16,18(21)-heptaene

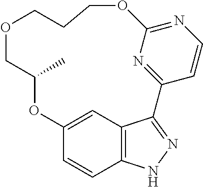 [SMILES: C[C@H]1COCCCOc2nccc(n2)-c2n[nH]c3ccc(cc23)O1]

Example 26 is prepared according to the synthesis route described in general Scheme C, following the same synthesis procedures as for Example 7.

### Preparation of Intermediate 165: (13S)-13-methyl-19-(oxan-2-yl)-7,11,14-trioxa-5,19,20,23-tetraazatetracyclo[13.5.2.1^{2,6}.0^{18,21}]tricosa-1(20),2(23),3,5,15(22),16, 18(21)-heptaene

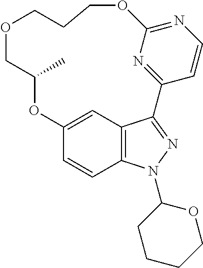 [SMILES: C[C@H]1COCCCOc2nccc(n2)-c2nn(C3CCCCO3)c3ccc(cc23)O1]

To a solution of 3-[2-[3-[(2R)-2-hydroxypropoxy]propoxy]pyrimidin-4-yl]-1-tetrahydropyran-2-yl-indazol-5-ol (240 mg, 0.560 mmol) in dry toluene (28 mL) purged with nitrogen for 5 min. was added (tributylphosphoranylidene)acetonitrile (220 μL, 0.840 mmol). The reaction mixture was purged with nitrogen for 5 min and the reaction mixture was stirred at 150° C. for 2 h under microware irradiations. The reaction mixture was cooled to RT, filtered over a pad of Celite and rinsed with ethyl acetate. The residue was diluted with ethyl acetate and washed with water and brine. The organic layer was dried over anhydrous magnesium sulfate, filtered and evaporated under reduced pressure. The residue was purified by silica gel column chromatography using heptane/ethyl acetate 1000 to 60/40 as eluent to afford (13S)-13-methyl-19-(oxan-2-yl)-7,11,14-trioxa-5,19,20,23-tetraazatetracyclo[13.5.2.1^{2,6}.0^{18,21}]tricosa-1(20), 2(23),3,5,15(22),16,18(21)-heptaene as a yellow solid.

LCMS method B: [M+H]⁺=411.0, t_{R}=1.135 min

### Preparation of Example 26: (13S)-13-methyl-7,11,14-trioxa-5,19,20,23-tetraazatetra cyclo[13.5.2.1^{2,6}.0^{18,21}]tricosa-1(20),2(23),3, 5,15(22),16,18(21)-heptaene

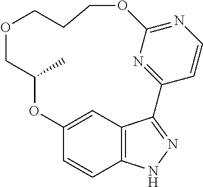 [SMILES: C[C@H]1COCCCOc2nccc(n2)-c2n[nH]c3ccc(cc23)O1]

HCl (4M solution in dioxane) (9.75 mL) was added to (13S)-13-methyl-19-(oxan-2-yl)-7,11,14-trioxa-5,19,20,23-tetraazatetracyclo[13.5.2.1^{2,6}.0^{18,21}]tricosa-1(20),2(23),3,5,15(22), 16,18(21)-heptaene (80 mg, 0.195 mmol). The reaction mixture was stirred at RT for 63 h. The solvent was evaporated reduced pressure and the residue was basified to pH=8 using a saturated aqueous NaHCO₃ solution. The aqueous layer was extracted with dichloromethane. The combined organic layers were dried over anhydrous magnesium sulfate, filtered and evaporated under reduced pressure. The residue was purified by silica gel column chromatography using heptane/ethyl acetate 100/0 to 50/50/ as eluent to afford (13S)-13-methyl-7,11,14-trioxa-5,19,20,23-tetraazatetracyclo[13.5.2.1^{2,6}.0^{18,21}]tricosa-1(20),2(23),3,5,15(22),16,18(21)-heptaene as a solid.

LCMS method E: [M+H]⁺=327.1, t_{R}=3.036 min

LCMS method D: [M+H]⁺=327.1, t_{R}=3.506 min

¹H NMR (400 MHz, d6-DMSO) δ 13.62 (s, 1H), 8.74 (d, J=2.3 Hz, 1H), 8.58 (d, J=5.1 Hz, 1H), 7.75 (d, J=5.1 Hz, 1H), 7.49 (d, J=8.9 Hz, 1H), 7.05 (dd, J=8.9, 2.3 Hz, 1H), 5.18 (ddd, J=12.2, 10.9, 4.5 Hz, 1H), 4.42-4.28 (m, 2H), 3.78 (dd, J=9.8, 6.5 Hz, 1H), 3.69-3.54 (m, 2H), 3.44 (dd, J=9.8, 3.5 Hz, 1H), 2.43-2.27 (m, 1H), 1.82 (s, 1H), 1.32 (d, J=6.5 Hz, 3H) ppm.

### Example 27: (13S)-4-(3-methoxyazetidin-1-yl)-13-methyl-7,11,14-trioxa-5,19,20,23-tetra azatetracyclo[13.5.2.1^{2,6}.0^{18,21}]tricosa-1(20),2(23),3,5,15(22),16,18(21) heptaene

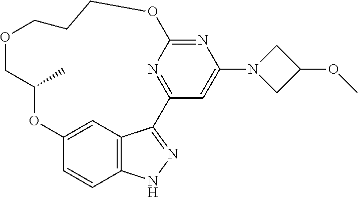 [SMILES: COC1CN(c2cc3nc(n2)OCCCOC[C@H](C)Oc2ccc4[nH]nc-3c4c2)C1]

Example 27 is prepared according to the synthesis route described in general Scheme E, following the same synthesis procedures as for Example 19.

To a solution of TFA (3 mL) in dichloromethane (3 mL) was added (13S)-4-(3-methoxyazetidin-1-yl)-13-methyl-19-(oxan-2-yl)-7,11,14-trioxa-5,19,20,23-tetraazatetracyclo [13.5.2.1^{2,6}.0^{18,21}]tricosa-1(20),2(23),3,5,15(22),16,18(21)-heptaene (300 mg, 0.605 mmol). The reaction mixture was stirred at RT for 2 h. The solvent was evaporated under reduced pressure and the residue was basified to pH=8 using a saturated aqueous NaHCO₃ solution. The aqueous layer was extracted with dichloromethane. The combined organic layers were dried over anhydrous magnesium sulfate, filtered and evaporated under reduced pressure. The residue was purified by silica gel column chromatography using heptane/ethyl acetate 100/0 to 40/60 as eluent to afford (13S)-4-(3-methoxyazetidin-1-yl)-13-methyl-7,11,14-trioxa-5,19,20,23-tetraazatetracyclo[13.5.2.1^{2,6}.0^{18,21}]tricosa-1(20),2(23),3,5,15(22),16,18(21)heptaene as a solid.

LCMS method E: [M+H]⁺=412.2, t_{R}=3.195 min

LCMS method D: [M+H]⁺=412.1, t_{R}=3.623 min

¹H NMR (400 MHz, DMSO) 13.38 (1H, s), 8.70 (1H, d, J=2.1 Hz), 7.47-7.43 (1H, m), 7.03-6.99 (1H, m), 6.74 (1H, m), 5.77 (1H, m), 5.15-5.06 (1H, m), 4.39-4.19 (4H, m), 3.88 (2H, d, J=9.3 Hz), 3.77 (1H, dd, J=6.3, 9.7 Hz), 3.65-3.54 (2H, m), 3.40 (1H, dd, J=3.9, 9.7 Hz), 3.26 (s, 3H), 2.41-2.27 (1H, m), 1.81-1.73 (1H, m), 1.32 (3H, d, J=6.5 Hz) ppm.

### Example 28: (13S)-13-methyl-7,10,14-trioxa-19,20,23-triazatetracyclo [13.5.2.1^{2,6}.0^{18,21}] tricosa-1(20),2(23),3,5,15(22),16,18(21)-heptaene

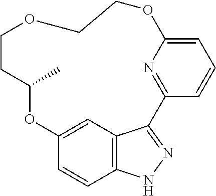 [SMILES: C[C@H]1CCOCCOc2cccc(n2)-c2n[nH]c3ccc(cc23)O1]

Example 28 is prepared according to the synthesis route described in general Scheme D.

### Preparation of Intermediate 166: [(1R)-3-bromo-1-methyl-propoxy]methylbenzene

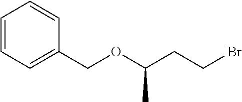

To a solution of (3R)-3-benzyloxybutan-1-ol (intermediate 128) (4.5 g, 24.97 mmol) and triphenylphosphine (7.21 g, 27.47 mmol) in dichloromethane (140 mL) at 0° C. was added dropwise carbon tetrachloride (9.11 g, 27.47 mmol) in dichloromethane (10 mL). The reaction mixture was stirred at RT for 4 h. The solvent was evaporated under reduced pressure. The residue was purified by silica gel column chromatography using cyclohexane/ethyl acetate 100/0 to 90/10 as eluent to afford [(1R)-3-bromo-1-methyl-propoxy]methylbenzene as a colorless oil.

¹H NMR (400 MHz, CDCl₃) 7.40-7.29 (5H, m), 4.64 (1H, d, J=11.4 Hz), 4.48 (1H, d, J=11.4 Hz), 3.82-3.74 (1H, m), 3.62-3.49 (2H, m), 2.20-2.09 (1H, m), 2.03-1.94 (1H, m), 1.27-1.25 (3H, m) ppm.

### Preparation of Intermediate 167: 2-[(3R)-3-benzyloxybutoxy]ethanol

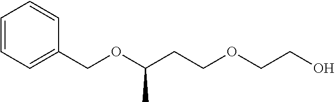 [SMILES: C[C@H](CCOCCO)OCc1ccccc1]

To a suspension of sodium hydride (60% dispersion in mineral oil) (362 mg, 9.042 mmol) in DMF (40 mL) at 0° C. was added ethylene glycol (4.6 mL, 82.2 mmol). The reaction mixture was stirred at RT for 15 min then cooled to 0° C. A solution of [(1R)-3-bromo-1-methyl-propoxy]methylbenzene (2 g, 8.22 mmol) in DMF (40 mL) was added dropwise. The reaction mixture was stirred at 0° C. for 30 min then RT overnight. The mixture was quenched with a saturated aqueous NH₄Cl solution and poured in ethyl acetate. The aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent to afford 2-[(3R)-3-benzyloxybutoxy]ethanol as a colorless oil.

¹H NMR (400 MHz, DMSO) 7.35-7.33 (5H, m), 4.58-4.51 (2H, m), 4.40 (1H, d, J=12.0 Hz), 3.65-3.58 (1H, m), 3.52-3.43 (4H, m), 3.39-3.34 (2H, m), 1.79-1.59 (2H, m), 1.16-1.14 (3H, m) ppm.

### Preparation of Intermediate 168: 2-[2-[(3R)-3-benzyloxybutoxy]ethoxy]-6-bromo-pyridine

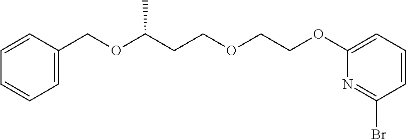 [SMILES: C[C@H](CCOCCOc1cccc(Br)n1)OCc1ccccc1]

To a solution of 6-bromopyridin-2-ol (375 mg, 2.16 mmol), 2-[(3R)-3-benzyloxybutoxy] ethanol (628 mg, 2.8 mmol) and triphenylphosphine (848 g, 3.23 mmol) in dry THF (15 mL) under argon atmosphere was added DIAD (637 μL, 3.23 mmol). The reaction mixture was stirred at RT overnight. The solvent was removed under reduced pressure. The residue was purified by silica gel column chromatography using cyclohexane/ethyl acetate 100/0 to 70/30 as eluent to afford 2-[2-[(3R)-3-benzyloxybutoxy]ethoxy]-6-bromo-pyridine as a colorless oil.

LCMS method F: [M+H]⁺=380.1-382.2, t_{R}=3.23 min

### Preparation of Intermediate 169: [3-[6-[2-[(3R)-3-benzyloxybutoxy]ethoxy]-2-pyridyl]-1-tetrahydropyran-2-yl-indazol-5-yl]oxy-tert-butyl-dimethyl-silane

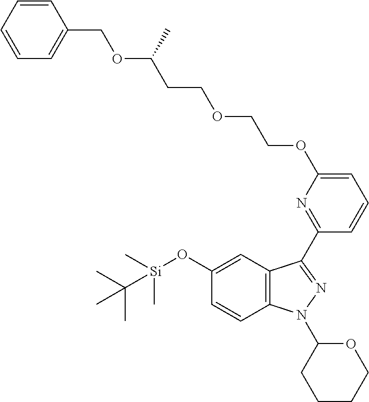 [SMILES: C[C@H](CCOCCOc1cccc(-c2nn(C3CCCCO3)c3ccc(O[Si](C)(C)C(C)(C)C)cc23)n1)OCc1ccccc1]

To a degassed solution of tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (940 mg, 2.05 mmol), 2-[2-[(3R)-3-benzyloxybutoxy]ethoxy]-6-bromo-pyridine (650 mg, 1.71 mmol), potassium phosphate tribasic (1.09 g, 5.13 mmol) in dioxane (30 mL) and water (3 mL) was added tetrakis(triphenylphosphine)palladium(0) (98 mg, 0.0855 mmol). The reaction mixture was stirred at 110° C. for 2 h. The reaction mixture was filtered over a pad of Celite and was washed with ethyl acetate. The filtrate was diluted with water and the aqueous layer was extracted with ethyl acetate. The combined organic layers were washed with water and brine, dried over anhydrous sodium sulfate and evaporated under reduced pressure. The residue was purified by silica gel column chromatography using cyclohexane/ethyl acetate 100/0 to 90/10 as eluent to afford [3-[6-[2-[(3R)-3-benzyloxybutoxy]ethoxy]-2-pyridyl]-1-tetrahydropyran-2-yl-indazol-5-yl]oxy-tert-butyl-dimethyl-silane as a colorless oil.

LCMS method M: [M+H]⁺=632.4, t_{R}=6.05 min

### Preparation of Intermediate 170: (2R)-4-[2-[[6-[5-[tert-butyl(dimethyl)silyl]oxy-1-tetrahydropyran-2-yl-indazol-3-yl]-2-pyridyl]oxy]ethoxy]butan-2-ol

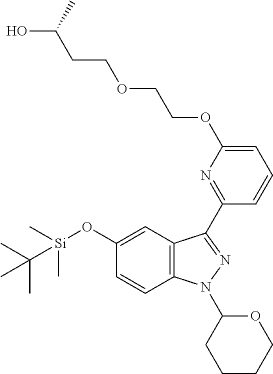 [SMILES: C[C@@H](O)CCOCCOc1cccc(-c2nn(C3CCCCO3)c3ccc(O[Si](C)(C)C(C)(C)C)cc23)n1]

To a solution of [3-[6-[2-[(3R)-3-benzyloxybutoxy]ethoxy]-2-pyridyl]-1-tetrahydropyran-2-yl-indazol-5-yl]oxy-tert-butyl-dimethyl-silane (801 mg, 1.27 mmol) in methanol (20 mL) at RT was added Pd/C 10% (80 mg). The reaction mixture was stirred under hydrogen atmosphere at 60° C. for 3 h. The reaction mixture was filtered and the solvent was removed under reduced pressure to afford (2R)-4-[2-[[6-[5-[tert-butyl(dimethyl)silyl]oxy-1-tetrahydropyran-2-yl-indazol-3-yl]-2-pyridyl]oxy]ethoxy]butan-2-ol as a colorless oil.

LCMS method F: [M+H]⁺=542.3, t_{R}=3.69 min

### Preparation of Intermediate 171: [(1R)-3-[2-[[6-[5-[tert-butyl(dimethyl)silyl]oxy-1-tetrahydropyran-2-yl-indazol-3-yl]-2-pyridyl]oxy]ethoxy]-1-methyl-propyl]methane sulfonate

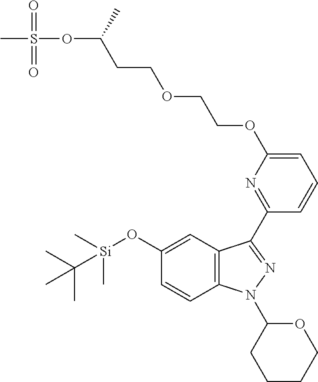 [SMILES: C[C@H](CCOCCOc1cccc(-c2nn(C3CCCCO3)c3ccc(O[Si](C)(C)C(C)(C)C)cc23)n1)OS(C)(=O)=O]

To a solution of (2R)-4-[2-[[6-[5-[tert-butyl(dimethyl)silyl]oxy-1-tetrahydropyran-2-yl-indazol-3-yl]-2-pyridyl]oxy]ethoxy]butan-2-ol (607 mg, 1.12 mmol) and triethylamine (313 μL, 2.24 mmol) in dichloromethane (15 mL) at 0° C. was added methanesulfonyl chloride (113 μL, 1.46 mmol) in dichloromethane (5 mL). The reaction mixture was stirred at RT for 1 h. The mixture was diluted with brine and extracted with dichloromethane. The combined organic layers were dried over anhydrous sodium sulfate and the solvent was removed under reduced pressure to afford [(1R)-3-[2-[[6-[5-[tert-butyl(dimethyl)silyl]oxy-1-tetrahydropyran-2-yl-indazol-3-yl]-2-pyridyl]oxy]ethoxy]-1-methyl-propyl]methanesulfonate as a yellow oil which was used in the next step without further purification.

LCMS method M: [M+H]⁺=620.3, t_{R}=4.95 min

### Preparation of Intermediate 172: (13S)-13-methyl-19-(oxan-2-yl)-7,10,14-trioxa-19,20,23-triazatetracyclo[13.5.2.1^{2,6}.0^{18,21}]tricosa-1(20),2(23),3,5,15(22),16,18(21)-heptaene

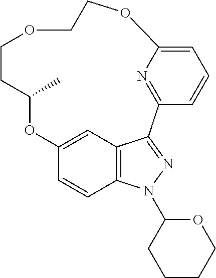 [SMILES: C[C@H]1CCOCCOc2cccc(n2)-c2nn(C3CCCCO3)c3ccc(cc23)O1]

To a suspension of sodium hydride (60% dispersion in mineral oil) (111 mg, 2.78 mmol) in anhydrous DMF (290 mL) at RT was added dropwise [(1R)-3-[2-[[6-[5-[tert-butyl(dimethyl)silyl]oxy-1-tetrahydropyran-2-yl-indazol-3-yl]-2-pyridyl]oxy]ethoxy]-1-methyl-propyl] methanesulfonate (574 mg, 0.93 mmol) in DMF (290 mL). The reaction mixture was stirred at RT overnight. The solvent was removed under reduced pressure and the solid was triturated in water. The solid was filtered, washed with water and dried under reduced pressure to afford (13S)-13-methyl-19-(oxan-2-yl)-7,10,14-trioxa-19,20,23-triazatetracyclo[13.5.2.1^{2,6}.0^{18,21}]tricosa-1(20),2(23),3,5,15(22),16,18(21)-heptaene as a pink powder.

LCMS method F: [M+H]⁺=410.3, t_{R}=3.45 min

### Preparation of Example 28: (13S)-13-methyl-7,10,14-trioxa-19,20,23-triazatetracyclo [13.5.2.1^{2,6}.0^{18,21}]tricosa-1(20),2(23),3,5,15(22),16,18(21)-heptaene

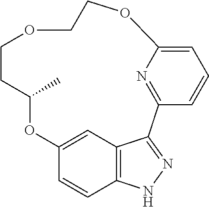 [SMILES: C[C@H]1CCOCCOc2cccc(n2)-c2n[nH]c3ccc(cc23)O1]

To a solution of (13S)-13-methyl-19-(oxan-2-yl)-7,10,14-trioxa-19,20,23-triazatetracyclo [13.5.2.1^{2,6}.0^{18,21}]tricosa-1(20),2(23),3,5,15(22),16,18(21)-heptaene (304 mg, 0.74 mmol) in methanol (31 mL) and water (4.6 mL) was added p-toluenesulfonic acid monohydrate (707 mg, 3.71 mmol). The reaction mixture was stirred at 65° C. for 2 h. The reaction mixture was concentrated under reduced pressure and the residue was neutralized by slow addition of saturated aqueous NaHCO₃ solution. The residue was diluted with ethyl acetate and the layers were separated. The aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent. The resulting solid was crystallized in diisopropylether to afford (13S)-13-methyl-7,10,14-trioxa-19,20,23-triazatetracyclo [13.5.2.1^{2,6}.0^{18,21}]tricosa-1(20),2(23),3,5,15(22),16,18(21)-heptaene as a powder.

LCMS method L: [M+H]⁺=326.3, t_{R}=2.75 min

LCMS method G: [M+H]⁺=326.3, t_{R}=2.72 min

¹H NMR (400 MHz, d6-DMSO) 13.24 (1H, s), 8.15 (1H, d, J=2.1 Hz), 7.80-7.77 (2H, m), 7.49-7.46 (1H, m), 6.99 (1H, dd, J=2.3, 8.9 Hz), 6.75-6.72 (1H, m), 5.06-4.99 (1H, m), 4.69-4.62 (1H, m), 4.26-4.14 (2H, m), 3.75-3.60 (3H, m), 2.41-2.34 (1H, m), 1.40-1.37 (4H, m) ppm.

### Example 29: (13S)-4,13-dimethyl-7,11,14-trioxa-5,19,20,23-tetraazatetracyclo[13.5.2.1^{2,6}.0^{18,21}]tricosa-1(20),2(23),3,5,15(22),16,18(21)-heptaene

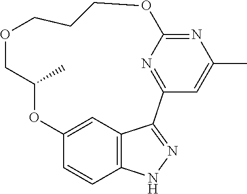 [SMILES: Cc1cc2nc(n1)OCCCOC[C@H](C)Oc1ccc3[nH]nc-2c3c1]

Example 29 is prepared according to the synthesis route described in general Scheme A, following the same synthesis procedures as for Example 19.

HCl (4M solution in dioxane) (22.05 mL) was added to (13S)-4,13-dimethyl-19-(oxan-2-yl)-7,11,14-trioxa-5,19,20,23-tetraazatetracyclo[13.5.2.1^{2,6}.0^{18,21}]tricosa-1(20),2(23),3,5,15(22), 16,18(21)-heptaene (187 mg, 0.441 mmol). The reaction mixture was stirred at RT for 15 h. The solvent was removed under reduced pressure and the residue was basified to pH=8 using a saturated aqueous NaHCO₃ solution. The aqueous layer was extracted with dichloromethane. The combined organic layers were dried over anhydrous magnesium sulfate, filtered and evaporated under reduced pressure. The residue was purified by silica gel column chromatography using heptane/ethyl acetate 100/0 to 50/50 as eluent to afford (13S)-4,13-dimethyl-7,11,14-trioxa-5,19,20,23-tetraazatetracyclo[13.5.2.1^{2,6}.0^{18,21}]tricosa-1(20),2(23),3, 5,15(22),16,18(21)-heptaene as a solid.

LCMS method E: [M+H]⁺=341.1, t_{R}=3.153 min

LCMS method D: [M+H]⁺=341.1, t_{R}=3.607 min

¹H NMR (400 MHz, d6-DMSO) 13.56 (s, 1H), 8.73 (d, J=2.4 Hz, 1H), 7.65 (s, 1H), 7.47 (d, J=8.9 Hz, 1H), 7.03 (dd, J=8.9, 2.3 Hz, 1H), 5.16 (td, J=11.6, 4.5 Hz, 1H), 4.40-4.25 (m, 2H), 3.77 (dd, J=9.8, 6.4 Hz, 1H), 3.67-3.52 (m, 2H), 3.42 (dd, J=9.8, 3.6 Hz, 1H), 2.42 (s, 3H), 2.39-2.26 (m, 1H), 1.80 (q, J=12.9, 9.0 Hz, 1H), 1.32 (d, J=6.5 Hz, 3H) ppm.

### Example 30: (13S)-13-methyl-7,10-dioxa-5,14,19,20,23-pentaazatetracyclo[13.5.2.1^{2,6}.0^{18,21}]tricosa-1(20),2(23),3,5,15(22),16,18(21)-heptaene

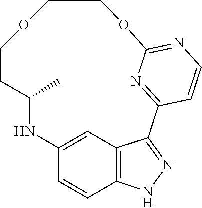 [SMILES: C[C@H]1CCOCCOc2nccc(n2)-c2n[nH]c3ccc(cc23)N1]

Example 30 is prepared according to the synthesis route described in general Scheme E.

### Preparation of Intermediate 173: 3-(2-methylsulfanylpyrimidin-4-yl)-1-tetrahydro pyran-2-yl-indazol-5-ol

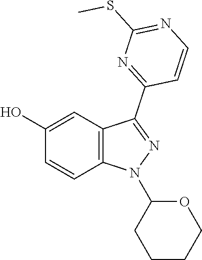 [SMILES: CSc1nccc(-c2nn(C3CCCCO3)c3ccc(O)cc23)n1]

To a solution of tert-butyl-dimethyl-[3-(2-methylsulfanylpyrimidin-4-yl)-1-tetrahydropyran-2-yl-indazol-5-yl]oxy-silane (intermediate 62) (3.57 g, 7.817 mmol) in THF (25 mL) at 0° C. under nitrogen atmosphere was added TBAF (1M in THF solution) (11.725 mL, 11.725 mmol). The reaction mixture was stirred at RT for 3 h. The reaction mixture was diluted with ethyl acetate and washed with a saturated aqueous NaHCO₃ solution. The organic layer was dried over anhydrous magnesium sulfate, filtered and evaporated removed under reduced pressure. The residue was purified by silica gel chromatography using heptane/ethyl acetate 100/0 to 70/30 as eluent to afford 3-(2-methylsulfanylpyrimidin-4-yl)-1-tetrahydropyran-2-yl-indazol-5-ol as a salmon solid.

LCMS method B: [M+H]⁺=343.0, t_{R}=0.908 min

### Preparation of Intermediate 174: [3-(2-methylsulfanylpyrimidin-4-yl)-1-tetrahydro pyran-2-yl-indazol-5-yl] trifluoromethanesulfonate

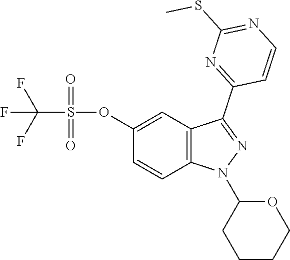 [SMILES: CSc1nccc(-c2nn(C3CCCCO3)c3ccc(OS(=O)(=O)C(F)(F)F)cc23)n1]

To a solution of 3-(2-methylsulfanylpyrimidin-4-yl)-1-tetrahydropyran-2-yl-indazol-5-ol (2.56 g, 7.476 mmol) and triethylamine (10.39 mL, 74.76 mmol) in dry dichloromethane (37 mL) at 0° C. under nitrogen atmosphere was added trifluoromethanesulfonic anhydride (2.52 mL, 14.95 mmol). The reaction mixture was stirred at RT for 3 h. Water was added and the mixture was extracted with dichloromethane. The combined organic layers were dried over anhydrous magnesium sulfate, filtered and evaporated under reduced pressure. The residue was purified by silica gel column chromatography using heptane/ethyl acetate 100:0 to 85:15 as eluent to afford [3-(2-methylsulfanylpyrimidin-4-yl)-1-tetrahydropyran-2-yl-indazol-5-yl]trifluoro methanesulfonate as a sand solid.

LCMS method B: [M+H]⁺=474.9, t_{R}=1.338 min

### Preparation of Intermediate 175: N-[3-(2-methylsulfanylpyrimidin-4-yl)-1-tetra hydropyran-2-yl-indazol-5-yl]-1,1-diphenyl-methanimine

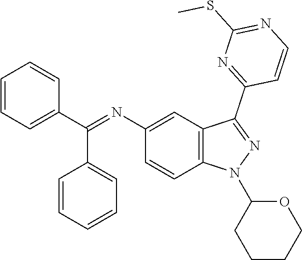 [SMILES: CSc1nccc(-c2nn(C3CCCCO3)c3ccc(N=C(c4ccccc4)c4ccccc4)cc23)n1]

To a solution of [3-(2-methylsulfanylpyrimidin-4-yl)-1-tetrahydropyran-2-yl-indazol-5-yl] trifluoromethanesulfonate (2.13 g, 4.50 mmol), cesium carbonate (2.93 g, 9.00 mmol) and diphenylmethanimine (1.51 ml, 9.00 mmol) in dioxane (24 mL) were added palladium (II) acetate (202 mg, 0.901 mmol) and Binap (56 mg, 0.09 mmol). The reaction mixture was degassed with nitrogen for 5 min and stirred at 100° C. for 6 h. The reaction mixture was diluted with ethyl acetate and water was added. The aqueous layer was extracted with ethyl acetate.

The combined organic layers were dried over anhydrous magnesium sulfate, filtered and evaporated under reduced pressure. The residue was purified by silica gel column chromatography using heptane/ethyl acetate 100:0 to 90:10 as eluent to afford N-[3-(2-methylsulfanylpyrimidin-4-yl)-1-tetrahydropyran-2-yl-indazol-5-yl]-1,1-diphenyl-methanimine as a yellow foam.

LCMS method B: [M+H]⁺=506.0, t_{R}=1.405 min

### Preparation of Intermediate 176: 3-(2-methylsulfanylpyrimidin-4-yl)-1-tetrahydro pyran-2-yl-indazol-5-amine

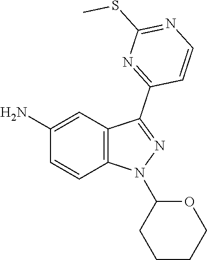 [SMILES: CSc1nccc(-c2nn(C3CCCCO3)c3ccc(N)cc23)n1]

A mixture of N-[3-(2-methylsulfanylpyrimidin-4-yl)-1-tetrahydropyran-2-yl-indazol-5-yl]-1,1-diphenyl-methanimine (1.67 g, 3.30 mmol), Pd/C 10% w/w (334 mg) and ammonium formate (4.166 g, 66.06 mmol) was heated at 70° C. for 16 h. The reaction mixture was diluted with ethyl acetate, filtered over a pad of celite and washed with ethyl acetate. The filtrate was concentrated under reduced pressure and the residue was diluted with ethyl acetate and washed with a saturated aqueous NaHCO₃ solution and extracted with ethyl acetate. The combined organic layers were dried over anhydrous magnesium sulfate, filtered and evaporated under reduced pressure. The residue was purified on silica gel column chromatography using heptane/ethyl acetate 100/0 to 40/60 as eluent to afford 3-(2-methylsulfanylpyrimidin-4-yl)-1-tetrahydropyran-2-yl-indazol-5-amine as a yellow foam.

LCMS method B: [M+H]⁺=342.0, t_{R}=0.765 min

### Preparation of Intermediate 177: N-[3-(2-methylsulfanylpyrimidin-4-yl)-1-tetra hydropyran-2-yl-indazol-5-yl]-2-nitro-benzenesulfonamide

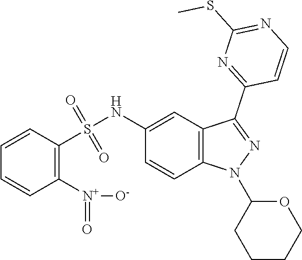 [SMILES: CSc1nccc(-c2nn(C3CCCCO3)c3ccc(NS(=O)(=O)c4ccccc4[N+](=O)[O-])cc23)n1]

To a solution of 3-(2-methylsulfanylpyrimidin-4-yl)-1-tetrahydropyran-2-yl-indazol-5-amine (488 mg, 1.429 mmol) in dioxane (7 mL) was added pyridine (231 μL, 2.85 mmol) and DMAP (17 mg, 0.143 mmol). The reaction mixture was stirred at 0° C. 2-nitro-benzenesulfonyl chloride (475 mg, 2.143 mmol) was added at 0° C. and the reaction mixture was stirred at 60° C. for 15 h. The reaction mixture was diluted with ethyl acetate and washed with a saturated aqueous NaHCO₃ solution. The aqueous phase was extracted with ethyl acetate and the combined organic layers were washed with brine, dried over anhydrous magnesium sulfate, filtered and evaporated under reduced pressure. The residue was triturated with heptane, filtered and dried to afford N-[3-(2-methylsulfanylpyrimidin-4-yl)-1-tetrahydropyran-2-yl-indazol-5-yl]-2-nitro-benzenesulfonamide as a beige solid which was used in the next step without further purification.

LCMS method B: [M+H]⁺=526.9, t_{R}=1.101 min

### Preparation of Intermediate 178: 2-[(3R)-3-benzyloxybutoxy]ethoxy-tert-butyl-dimethyl-silane

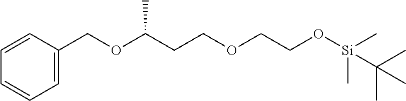 [SMILES: C[C@H](CCOCCO[Si](C)(C)C(C)(C)C)OCc1ccccc1]

To a solution of (R)-3-(benzyloxy)butan-1-ol (intermediate 128) (11.16 g, 61.914 mmol) in dry THF (186.0 mL) at 0° C. and under nitrogen atmosphere was added sodium hydride (7.43 g, 185.742 mmol, 60% in mineral oil) and the mixture was stirred at 0° C. for 1 h. 2-Bromoethoxy-t-butyl dimethylsilane (15.87 mL, 74.297 mmol) in dry THF (124.0 mL) was added and the reaction mixture was stirred at 80° C. for 15 h. The reaction mixture was cooled to 0° C., diluted with ethyl acetate and quenched with a saturated aqueous NH₄Cl solution and washed with brine (×3). The organic phase was dried over magnesium sulfate, filtered and the solvent was removed under reduced pressure. The crude product was purified by flash column chromatography on silica gel using a gradient of n-heptane/ethyl acetate (99:1 to 90:10) as eluents. The desired fractions were combined and the solvent was removed under reduced pressure to afford 2-[(3R)-3-benzyloxybutoxy]ethoxy-tert-butyl-dimethyl-silane as a yellow oil.

LCMS method E: [M+H]⁺=339.2, t_{R}=5.151 min

### Preparation of Intermediate 179: (R)-4-(2-((tert-butyldimethylsilyl)oxy)ethoxy) butan-2-ol

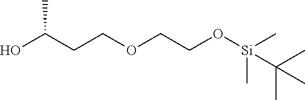 [SMILES: C[C@@H](O)CCOCCO[Si](C)(C)C(C)(C)C]

To a solution of 2-[(3R)-3-benzyloxybutoxy]ethoxy-tert-butyl-dimethyl-silane (3.5 g, 10.33 mmol) in ethyl acetate (50 mL) was added palladium 10% w on carbon (700 mg, 20% w/w). The reaction mixture was stirred under hydrogen atmosphere at RT for 15 h. The reaction mixture was filtered through a pad of celite, rinsed with ethyl acetate and the solvent was evaporated under reduced pressure to afford (R)-4-(2-((tert-butyldimethylsilyl)oxy)ethoxy) butan-2-ol as a yellow oil which was used in the next step without further purification.

LCMS method B: [M+H]⁺=249.1, t_{R}=not detected

### Preparation of Intermediate 180: N-[(1S)-3-[2-[tert-butyl(dimethyl)silyl]oxyethoxy]-1-methyl-propyl]-N-[3-(2-methylsulfanylpyrimidin-4-yl)-1-tetrahydropyran-2-yl-indazol-5-yl]-2-nitro-benzenesulfonamide

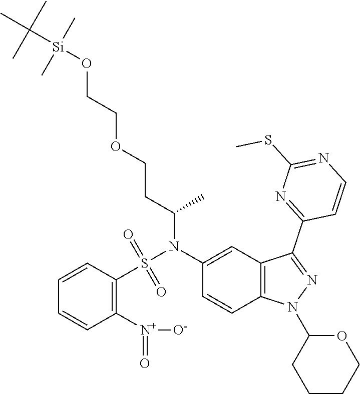 [SMILES: CSc1nccc(-c2nn(C3CCCCO3)c3ccc(N([C@@H](C)CCOCCO[Si](C)(C)C(C)(C)C)S(=O)(=O)c4ccccc4[N+](=O)[O-])cc23)n1]

To a solution of N-[3-(2-methylsulfanylpyrimidin-4-yl)-1-tetrahydropyran-2-yl-indazol-5-yl]-2-nitro-benzenesulfonamide (intermediate 177) (515 mg, 0.978 mmol) in dry THF (9 mL) under nitrogen atmosphere was added (R)-4-(2-((tert-butyldimethylsilyl)oxy)ethoxy)butan-2-ol (intermediate 179) (292 mg, 1.174 mmol) and triphenylphosphine (513 mg, 1.956 mmol). The reaction mixture was cooled down to 0° C. and diisopropyl azodicarboxylate (384 μL, 1.956 mmol) was added dropwise. The reaction mixture was stirred at RT for 4 h. The solvent was removed under reduced pressure and the residue was purified by silica gel column chromatography using heptane/ethyl acetate 100/0 to 75/25 as eluent to afford N-[(1S)-3-[2-[tert-butyl(dimethyl)silyl]oxyethoxy]-1-methyl-propyl]-N-[3-(2-methylsulfanylpyrimidin-4-yl)-1-tetrahydropyran-2-yl-indazol-5-yl]-2-nitro-benzenesulfonamide as a salmon solid.

LCMS method B: [M+H]⁺=757.0, t_{R}=1.573 min

### Preparation of Intermediate 181: N-[(1S)-3-(2-hydroxyethoxy)-1-methyl-propyl]-N-[3-(2-methylsulfanylpyrimidin-4-yl)-1-tetrahydropyran-2-yl-indazol-5-yl]-2-nitro-benzenesulfonamide

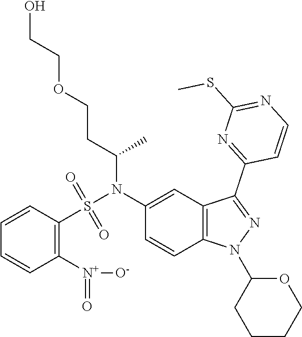 [SMILES: CSc1nccc(-c2nn(C3CCCCO3)c3ccc(N([C@@H](C)CCOCCO)S(=O)(=O)c4ccccc4[N+](=O)[O-])cc23)n1]

To a solution of N-[(1S)-3-[2-[tert-butyl(dimethyl)silyl]oxyethoxy]-1-methyl-propyl]-N-[3-(2-methylsulfanylpyrimidin-4-yl)-1-tetrahydropyran-2-yl-indazol-5-yl]-2-nitro-benzene sulfonamide (850 mg, 1.123 mmol) in THF (11 mL) at 0° C. under nitrogen atmosphere was added TBAF (1M solution in THF) (1.348 mL, 1.348 mmol). The reaction mixture was stirred at RT for 2 h. The reaction mixture was diluted with ethyl acetate and washed with a saturated aqueous NaHCO₃ solution. The organic layer was dried over anhydrous magnesium sulfate, filtered and the solvent was removed under reduced pressure. The residue was purified by silica gel column chromatography using heptanet/ethyl acetate 100/0 to 40/60 as eluent to afford N-[(1S)-3-(2-hydroxyethoxy)-1-methyl-propyl]-N-[3-(2-methylsulfanylpyrimidin-4-yl)-1-tetra hydropyran-2-yl-indazol-5-yl]-2-nitro-benzenesulfonamide as a colorless oil.

LCMS method B: [M+H]⁺=643.0, t_{R}=1.128 min

### Preparation of Intermediate 182: N-[(1S)-3-(2-hydroxyethoxy)-1-methyl-propyl]-N-[3-(2-methylsulfonylpyrimidin-4-yl)-1-tetrahydropyran-2-yl-indazol-5-yl]-2-nitro-benzenesulfonamide

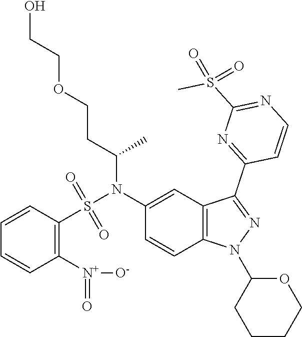 [SMILES: C[C@@H](CCOCCO)N(c1ccc2c(c1)c(-c1ccnc(S(C)(=O)=O)n1)nn2C1CCCCO1)S(=O)(=O)c1ccccc1[N+](=O)[O-]]

A solution of N-[(1S)-3-(2-hydroxyethoxy)-1-methyl-propyl]-N-[3-(2-methylsulfanyl pyrimidin-4-yl)-1-tetrahydropyran-2-yl-indazol-5-yl]-2-nitro-benzenesulfonamide (425 mg, 0.661 mmol) in dichloromethane (6 mL) at 0° C. was added 3-chloroperbenzoic acid (380 mg, 1.652 mmol). The reaction mixture was stirred at RT for 48 h. The mixture was diluted with dichloromethane and filtered. The filtrate was washed with a saturated aqueous NaHCO₃ solution and the organic layer was dried over anhydrous magnesium sulfate, filtered and the solvent was removed under reduced pressure. The residue was purified by silica gel column chromatography using heptane/ethyl acetate 100/0 to 0/100 as eluent to afford N-[(1S)-3-(2-hydroxyethoxy)-1-methyl-propyl]-N-[3-(2-methylsulfonylpyrimidin-4-yl)-1-tetrahydropyran-2-yl-indazol-5-yl]-2-nitro-benzenesulfonamide as a white foam.

LCMS method B: [M+H]⁺=674.9, t_{R}=0.946 min

### Preparation of Intermediate 183: (13S)-13-methyl-14-(2-nitrobenzenesulfonyl)-19-(oxan-2-yl)-7,10-dioxa-5,14,19,20,23-pentaazatetracyclo[13.5.2.1^{2,6}.0^{18,21}]tricosa-1(20),2(23),3,5,15(22),16,18(21)-heptaene

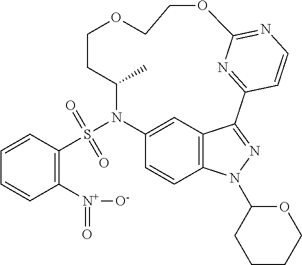 [SMILES: C[C@H]1CCOCCOc2nccc(n2)-c2nn(C3CCCCO3)c3ccc(cc23)N1S(=O)(=O)c1ccccc1[N+](=O)[O-]]

To a solution sodium hydride (60% dispersion in mineral oil) (41 mg, 1.023 mmol) in dry THF (17 mL) at 60° C. under nitrogen atmosphere was added dropwise a solution of N-[(1S)-3-(2-hydroxyethoxy)-1-methyl-propyl]-N-[3-(2-methylsulfonylpyrimidin-4-yl)-1-tetrahydropyran-2-yl-indazol-5-yl]-2-nitro-benzenesulfonamide (230 mg, 0.341 mmol) in dry THF (17 mL). The reaction mixture was stirred at 60° C. for 3 h. The mixture was cooled at 0° C. diluted with ethyl acetate and quenched by water. The aqueous layer was extracted with ethyl acetate. The combined organic layers were dried over anhydrous magnesium sulfate, filtered and evaporated under reduced pressure. The residue was purified by silica gel column chromatography using heptane/ethyl acetate 100/0 to 40/60 as eluent to afford (13S)-13-methyl-14-(2-nitrobenzenesulfonyl)-19-(oxan-2-yl)-7,10-dioxa-5,14,19,20,23-pentaazatetracyclo[13.5.2.1^{2,6}.0^{18,21}]tricosa-1(20),2(23),3,5,15(22),16,18(21)-heptaene as a yellowish solid.

LCMS method B: [M+H]⁺=595.0, t_{R}=1.152 min

### Preparation of Intermediate 184: (13S)-13-methyl-19-(oxan-2-yl)-7,10-dioxa-5,14,19,20,23-pentaazatetracyclo[13.5.2.1^{2,6}.0^{18,21}]tricosa-1(20),2(23),3,5,15(22),16, 18(21)-heptaene

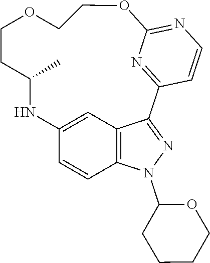 [SMILES: C[C@H]1CCOCCOc2nccc(n2)-c2nn(C3CCCCO3)c3ccc(cc23)N1]

To a suspension of (13S)-13-methyl-14-(2-nitrobenzenesulfonyl)-19-(oxan-2-yl)-7,10-dioxa-5,14,19,20,23-pentaazatetracyclo[13.5.2.1^{2,6}.0^{18,21}]tricosa-1(20),2(23),3,5,15(22),16,18(21)-heptaene (140 mg, 0.235 mmol) and cesium carbonate (230 mg, 0.705 mmol) in DMF (33 mL) at 0° C. was added thiophenol (72 μL, 0.705 mmol). The reaction mixture was stirred at RT for 2 h. The mixture was diluted with ethyl acetate and 1N aqueous NaOH solution. The aqueous layer was extracted with ethyl acetate. The combined organic layers were dried over anhydrous magnesium sulfate, filtered and evaporated under reduced pressure. The residue was purified by silica gel column chromatography using heptane/ethyl acetate 100/0 to 50/50 as eluent to afford (13S)-13-methyl-19-(oxan-2-yl)-7,10-dioxa-5,14,19,20,23-pentaazatetracyclo[13.5.2.1^{2,6}.0^{18,21}]tricosa-1(20),2(23),3,5,15(22),16,18(21)-heptaene as a yellow solid.

LCMS method B: [M+H]⁺=410.1, t_{R}=1.028 min

### Preparation of Example 30: (13S)-13-methyl-7,10-dioxa-5,14,19,20,23-pentaazatetra cyclo[13.5.2.1^{2,6}.0^{18,21}]tricosa-1(20),2(23),3,5,15(22),16,18(21)-heptaene

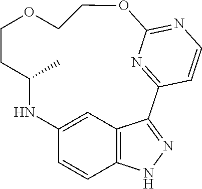 [SMILES: C[C@H]1CCOCCOc2nccc(n2)-c2n[nH]c3ccc(cc23)N1]

To a solution of (13S)-13-methyl-19-(oxan-2-yl)-7,10-dioxa-5,14,19,20,23-pentaazatetracyclo [13.5.2.1^{2,6}.0^{18,21}]tricosa-1(20),2(23),3,5,15(22),16,18(21)-heptaene (48 mg, 0.097 mmol) in dichloromethane (750 μL) at 0° C. was added TFA (0.75 mL). The reaction was stirred at RT for 5 h. The reaction mixture was concentrated under reduced pressure and co-evaporated with methanol. The resulting solid was diluted with dichloromethane, cooled at 0° C. and neutralized with a saturated aqueous NaHCO₃ solution. The aqueous layer was extracted with dichloromethane. The combined organic layers were dried over anhydrous magnesium sulfate, filtered and evaporated under reduced pressure. The residue was purified by silica el column chromatography using dichloromethane/ethyl acetate 100/0 to 70/30 as eluent to afford (13S)-13-methyl-7,10-dioxa-5,14,19,20,23-pentaazatetracyclo[13.5.2.1^{2,6}.0^{18,21}]tricosa-1(20),2(23), 3,5,15(22),16,18(21)-heptaene as a solid.

LCMS method E: [M+H]⁺=411.1, t_{R}=2.409 min

LCMS method D: [M+H]⁺=411.1, t_{R}=3.355 min

¹H NMR (400 MHz, d6-DMSO) 13.12 (s, 1H), 7.53 (d, J=2.1 Hz, 1H), 7.31 (d, J=8.9 Hz, 1H), 6.85 (dd, J=9.0, 2.1 Hz, 1H), 6.68 (s, 1H), 5.41 (d, J=6.5 Hz, 1H), 4.89 (td, J=10.4, 5.3 Hz, 1H), 4.35 (tt, J=6.2, 3.9 Hz, 1H), 4.30-4.17 (m, 3H), 4.10 (td, J=10.6, 5.1 Hz, 1H), 3.85 (dd, J=9.7, 3.8 Hz, 2H), 3.72 (td, J=11.9, 2.5 Hz, 1H), 3.69-3.55 (m, 1H), 3.54-3.41 (m, 2H), 3.26 (s, 3H), 2.47-2.38 (m, 1H), 1.24 (d, J=6.5 Hz, 3H), 1.01-0.89 (m, 1H) ppm.

### Example 31: 7,7-dimethyl-9,13-dioxa-4,5,18,19-tetraazatetracyclo[12.5.2.1^{2,5}.0^{17,20}] docosa-1(19),2(22),3,14(21),15,17(20)-hexaene

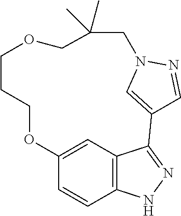 [SMILES: CC1(C)COCCCOc2ccc3[nH]nc(c3c2)-c2cnn(c2)C1]

Example 31 is prepared according to the synthesis route described in general Scheme D.

### Preparation of Intermediate 185: 3-(3-(benzyloxy)propoxy)-2,2-dimethylpropan-1-ol

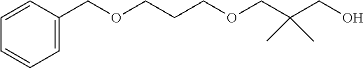 [SMILES: CC(C)(CO)COCCCOCc1ccccc1]

To a solution of 2,2-dimethylpropane-1,3-diol (4.95 g, 47.53 mmol) in dry DMF (75 mL) at 0° C. under nitrogen atmosphere was added sodium hydride (60% dispersion in mineral oil) (2.016 g, 50.41 mmol). The reaction mixture was stirred at 0° C. for 30 min then 3-bromopropoxymethylbenzene (3.3 g, 14.403 mmol) was added. The reaction mixture was stirred at RT for 16 h. The reaction mixture was diluted with dichloromethane and water. The aqueous layer was extracted with dichloromethane. The combined organic layers were washed with brine, dried over anhydrous magnesium sulfate, filtered and evaporated under reduced pressure. The residue was purified by silica gel column chromatography using heptane/ethyl acetate 100/0 to 85/15 as eluent to afford 3-(3-(benzyloxy)propoxy)-2,2-dimethylpropan-1-ol as a colorless oil.

LCMS method B: [M+H]⁺=253.1, t_{R}=0.886 min

### Preparation of Intermediate 186: 1-(3-(3-(benzyloxy)propoxy)-2,2-dimethylpropyl)-4-bromo-pyrazole

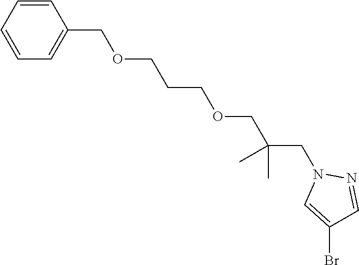 [SMILES: CC(C)(COCCCOCc1ccccc1)Cn1cc(Br)cn1]

To a solution of 4-bromopyrazole (1.60 g, 10.89 mmol) and 3-(3-(benzyloxy)propoxy)-2,2-dimethylpropan-1-ol (2.5 g, 9.90 mmol) in dry toluene (30 mL) was added (tributyl phosphoranylidene)acetonitrile (3.89 mL, 14.85 mmol). The reaction mixture was stirred at 150° C. for 2 h under microwave irradiations. The solvent was removed under reduced pressure and the residue was purified by silica gel column chromatography using heptane/ethyl acetate 100/0 to 90/10 as eluent to afford 1-(3-(3-(benzyloxy)propoxy)-2,2-dimethylpropyl)-4-bromo-pyrazole as a colorless oil.

LCMS method E: [M+H]⁺=381.1, t_{R}=4.48 min

### Preparation of Intermediate 187: [3-[1-[3-(3-benzyloxypropoxy)-2,2-dimethyl-propyl]pyrazol-4-yl]-1-tetrahydropyran-2-yl-indazol-5-yl]oxy-tert-butyl-dimethyl-silane

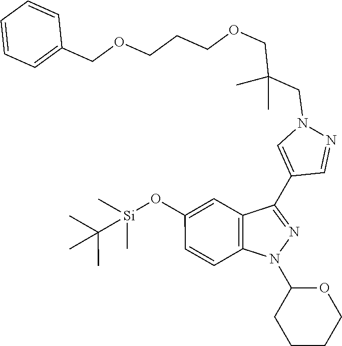 [SMILES: CC(C)(COCCCOCc1ccccc1)Cn1cc(-c2nn(C3CCCCO3)c3ccc(O[Si](C)(C)C(C)(C)C)cc23)cn1]

To a degassed solution of tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (1 g, 2.18 mmol), 1-[3-(3-benzyloxypropoxy)-2,2-dimethyl-propyl]-4-bromo-pyrazole (995 mg, 2.62 mmol) and potassium phosphate tribasic (1.386 g, 6.54 mmol) in dioxane (22 mL) and water (0.5 mL) were added tetrakis(triphenylphosphine)palladium(0) (127 mg, 0.11 mmol) and 2-dicyclohexyl phosphino-2′,4′,6′-triisopropylbiphenyl (105 mg, 0.22 mmol). The reaction mixture was stirred at 150° C. for 3 h. The reaction mixture was filtered over a pad of Celite and washed with ethyl acetate. The filtrate was diluted with water and extracted with ethyl acetate. The combined organic layers were washed with water, brine, dried over anhydrous magnesium sulfate, filtered and evaporated reduced pressure. The residue was purified by silica gel column chromatography using dichloromethane/methanol 100/0 to 98/2 as eluent to afford [3-[1-[3-(3-benzyloxypropoxy)-2,2-dimethyl-propyl]pyrazol-4-yl]-1-tetrahydropyran-2-yl-indazol-5-yl]oxy-tert-butyl-dimethyl-silane as a green oil.

LCMS method F: [M+H]⁺=633.4, t_{R}=3.98 min

### Preparation of Intermediate 188: 3-[3-[4-[5-[tert-butyl(dimethyl)silyl]oxy-1-tetra hydropyran-2-yl-indazol-3-yl]pyrazol-1-yl]-2,2-dimethyl-propoxy]propan-1-ol

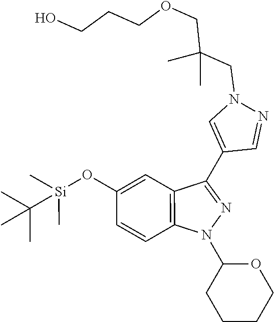 [SMILES: CC(C)(COCCCO)Cn1cc(-c2nn(C3CCCCO3)c3ccc(O[Si](C)(C)C(C)(C)C)cc23)cn1]

To a solution of [3-[1-[3-(3-benzyloxypropoxy)-2,2-dimethyl-propyl]pyrazol-4-yl]-1-tetra hydropyran-2-yl-indazol-5-yl]oxy-tert-butyl-dimethyl-silane (1.07 g, 1.69 mmol) in ethanol (25 mL) at RT was added palladium hydroxide on carbon (160 mg). The reaction mixture was stirred under hydrogen atmosphere at RT overnight. The reaction mixture filtered and the filtrate was concentrated under reduced pressure. The residue was purified by silica gel column chromatography using cyclohexane/ethyl acetate 100/0 to 20/80 as eluent to afford 3-[3-[4-[5-[tert-butyl(dimethyl)silyl]oxy-1-tetrahydropyran-2-yl-indazol-3-yl]pyrazol-1-yl]-2,2-dimethyl-propoxy]propan-1-ol as a colorless oil.

LCMS method F: [M+H]⁺=543.3, t_{R}=3.54 min

### Preparation of Intermediate 189: 3-[3-[4-[5-[tert-butyl(dimethyl)silyl]oxy-1-tetra hydropyran-2-yl-indazol-3-yl]pyrazol-1-yl]-2,2-dimethyl-propoxy]propylmethane sulfonate

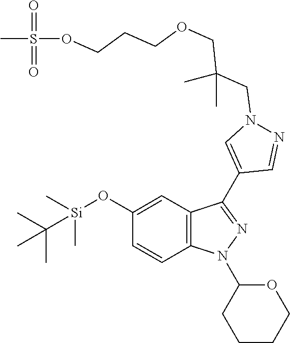 [SMILES: CC(C)(COCCCOS(C)(=O)=O)Cn1cc(-c2nn(C3CCCCO3)c3ccc(O[Si](C)(C)C(C)(C)C)cc23)cn1]

To a solution of 3-[3-[4-[5-[tert-butyl(dimethyl)silyl]oxy-1-tetrahydropyran-2-yl-indazol-3-yl]pyrazol-1-yl]-2,2-dimethyl-propoxy]propan-1-ol (700 mg, 1.29 mmol) and triethylamine (360 μL, 2.58 mmol) in dichloromethane (15 mL) at 0° C. was added dropwise methanesulfonyl chloride (120 μL, 1.67 mmol). The reaction mixture was stirred at RT for 2 h. The mixture was diluted with water and the aqueous layer was extracted with dichloromethane. The combined organic layers were washed with brine, dried over anhydrous magnesium sulfate, filtered and evaporated under reduced pressure to provide 3-[3-[4-[5-[tert-butyl(dimethyl)silyl]oxy-1-tetra hydropyran-2-yl-indazol-3-yl]pyrazol-1-yl]-2,2-dimethyl-propoxy]propylmethanesulfonate as a colorless oil which was used in the next step without further purification.

LCMS method F: [M+H]⁺=621.3, t_{R}=3.59 min

### Preparation of Intermediate 190: 7,7-dimethyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene

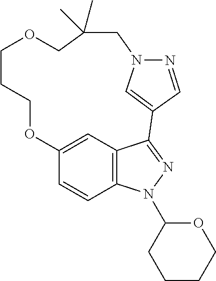 [SMILES: CC1(C)COCCCOc2ccc3c(c2)c(nn3C2CCCCO2)-c2cnn(c2)C1]

To a suspension of cesium carbonate (1.257 g, 3.87 mmol) in anhydrous DMF (250 mL) at 80° C. was added dropwise 3-[3-[4-[5-[tert-butyl(dimethyl)silyl]oxy-1-tetrahydropyran-2-yl-indazol-3-yl]pyrazol-1-yl]-2,2-dimethyl-propoxy]propylmethanesulfonate (800 mg, 1.29 mmol) in DMF (50 mL). The reaction mixture was stirred at 90° C. for 3 h. The reaction mixture filtered over a pad of Celite and washed with ethyl acetate. The filtrate was concentrated under reduced pressure. The residue was purified by filtration over a short pad of silica gel eluting with dichloromethane/methanol 96/4 as eluent to afford 7,7-dimethyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene as a yellow solid.

LCMS method F: [M+H]⁺=411.3, t_{R}=2.90 min

### Preparation of Example 31: 7,7-dimethyl-9,13-dioxa-4,5,18,19-tetraazatetracyclo [12.5.2.1^{2,5}.0^{17,20}]docosa-1(19),2(22),3,14(21),15,17(20)-hexaene

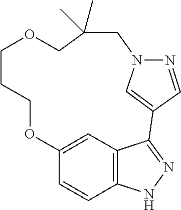 [SMILES: CC1(C)COCCCOc2ccc3[nH]nc(c3c2)-c2cnn(c2)C1]

To a solution of 7,7-dimethyl-18-(oxan-2-yl)-9,13-dioxa-4,5,18,19-tetraazatetracyclo [12.5.2.1^{2,5}.0^{17,20}]docosa-1(19),2(22),3,14(21),15,17(20)-hexaene (426 mg, 1.04 mmol) in methanol (58 mL) and water (7.5 mL) was added p-toluenesulfonic acid monohydrate (987 mg, 5.20 mmol). The reaction mixture was stirred at 65° C. for 4 h. The solvent was evaporated under reduced pressure and the residue was neutralized by slow addition of a saturated aqueous NaHCO₃ solution. The resulting suspension was diluted with ethyl acetate. The aqueous layer was extracted with ethyl acetate. The combined organic layers were washed with water and brine, dried over anhydrous magnesium sulfate, filtered and evaporated under reduced pressure. The residue was purified by silica gel column chromatography using dichloromethane/methanol 100/0 to 95/5 as eluent to afford 7,7-dimethyl-9,13-dioxa-4,5,18,19-tetraazatetracyclo[12.5.2.1^{2,5}.0^{17,20}]docosa-1(19),2(22),3,14(21),15,17(20)-hexaene as a solid.

LCMS method E: [M+H]⁺=327.2, t_{R}=2.39 min

LCMS method D: [M+H]⁺=327.3, t_{R}=2.26 min

¹H NMR (400 MHz, d6-DMSO) 12.71 (1H, s), 8.43 (1H, s), 7.63 (1H, s), 7.44-7.39 (2H, m), 6.95 (1H, dd, J=2.3, 8.9 Hz), 4.27 (2H, d, J=8.0 Hz), 4.20 (2H, s), 3.58 (2H, d, J=5.2 Hz), 3.30 (2H, s), 2.13-2.05 (2H, m), 0.87 (6H, s) ppm.

### Example 32: (12S)-12-methyl-9,13-dioxa-4,5,18,19-tetraazatetracyclo[12.5.2.1^{2,5}.0^{17,20}] docosa-1(19),2(22),3,14(21),15,17(20)-hexaene

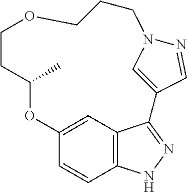 [SMILES: C[C@H]1CCOCCCn2cc(cn2)-c2n[nH]c3ccc(cc23)O1]

Example 32 is prepared according to the synthesis route described in general Scheme D.

### Preparation of Intermediate 191: [(1R)-3-bromo-1-methyl-propoxy]methylbenzene

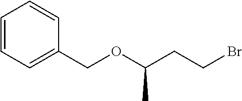

To a solution of (3R)-3-benzyloxybutan-1-ol (intermediate 128) (1.600 g, 8.88 mmol) and PPh3 (2.562 g, 9.77 mmol) in dichloromethane (20 mL) was added dropwise at 0° C. CBr4 (3.239 g, 9.77 mmol) in dichloromethane (10 mL). The reaction mixture was stirred at RT overnight. The solvent was removed under reduced pressure and the residue was purified by flash-column (40 g silica Macherey Nagel) chromatography (cyclohexane/ethyl acetate, 100:0 to 80:20). The desired fractions were combined and the solvent was removed under reduced pressure affording [(1R)-3-bromo-1-methyl-propoxy]methylbenzene as a colorless liquid.

¹H NMR (400 MHz, CDCl₃) 7.42-7.26 (5H, m), 4.64 (1H, d, J=11.4 Hz), 4.50-4.46 (1H, d, J=11.4 Hz), 3.82-3.74 (1H, m), 3.62-3.49 (2H, m), 2.18-1.94 (2H, m), 1.27-1.25 (3H, d, J=6.1 Hz) ppm.

### Preparation of Intermediate 192: 1-[3-[(3R)-3-benzyloxybutoxy]propyl]-4-bromo-pyrazole

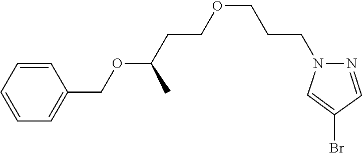 [SMILES: C[C@H](CCOCCCn1cc(Br)cn1)OCc1ccccc1]

To a solution of 3-(4-bromopyrazol-1-yl)propan-1-ol (900 mg, 4.39 mmol) in anhydrous DMF (10 mL) at 0° C. was added portionwise sodium hydride (60% dispersion in mineral oil) (211 mg, 5.27 mmol). The reaction was stirred 20 min then [(1R)-3-bromo-1-methyl-propoxy] methylbenzene (1.12 g, 4.61 mmol) in DMF (5 mL) was added dropwise at −40° C. The reaction mixture was stirred at RT overnight then quenched by addition of ethanol. The solvent was removed under reduced pressure. The residue was diluted with brine and the aqueous layer was extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 80/20 as eluent to afford 1-[3-[(3R)-3-benzyloxybutoxy]propyl]-4-bromo-pyrazole as a colorless oil.

LCMS method F: [M+H]⁺=367.1-369.1, t_{R}=2.99 min

### Preparation of Intermediate 193: [3-[1-[3-[(3R)-3-benzyloxybutoxy]propyl]pyrazol-4-yl]-1-tetrahydro pyran-2-yl-indazol-5-yl]oxy-tert-butyl-dimethyl-silane

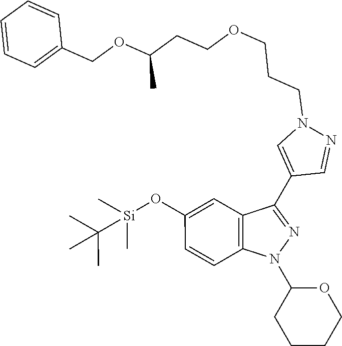 [SMILES: C[C@H](CCOCCCn1cc(-c2nn(C3CCCCO3)c3ccc(O[Si](C)(C)C(C)(C)C)cc23)cn1)OCc1ccccc1]

To a solution of tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (769 mg, 1.68 mmol) in dioxane (4 mL) and water (0.4 mL) at RT were added 1-[3-[(3R)-3-benzyloxybutoxy]propyl]-4-bromo-pyrazole (440 mg, 1.20 mmol), potassium phosphate tribasic (763 mg, 3.59 mmol), XPhos (57 mg, 0.12 mmol) and tetrakis(triphenylphosphine)palladium(0) (69 mg, 0.06 mmol). The reaction mixture was stirred under microwave irradiations at 90° C. for 2 h. The residue was diluted with brine and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 70/30 as eluent to afford [3-[1-[3-[(3R)-3-benzyloxybutoxy]propyl]pyrazol-4-yl]-1-tetrahydro pyran-2-yl-indazol-5-yl]oxy-tert-butyl-dimethyl-silane as an orange oil.

LCMS method F: [M+H]⁺=619.4, t_{R}=3.85 min

### Preparation of Intermediate 194: (2R)-4-[3-[4-[5-[tert-butyl(dimethyl)silyl]oxy-1-tetrahydropyran-2-yl-indazol-3-yl]pyrazol-1-yl]propoxy]butan-2-ol

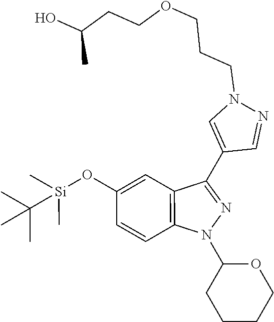 [SMILES: C[C@@H](O)CCOCCCn1cc(-c2nn(C3CCCCO3)c3ccc(O[Si](C)(C)C(C)(C)C)cc23)cn1]

To a solution of [3-[1-[3-[(3R)-3-benzyloxybutoxy]propyl]pyrazol-4-yl]-1-tetrahydropyran-2-yl-indazol-5-yl]oxy-tert-butyl-dimethyl-silane (430 mg, 0.69 mmol) in ethyl acetate (10 mL) at RT was added palladium 10% on carbon (21 mg). The reaction mixture was stirred under hydrogen atmosphere at RT overnight then at 50° C. for 6 h. The reaction mixture was filtered and palladium hydroxyde on carbon (21 mg) was added to the filtrate. The reaction mixture was stirred under hydrogen atmosphere at RT for 48 h. The reaction mixture was filtered and the filtrate was concentrated under reduced pressure. The residue was purified by silica gel column chromatography using dichloromethane/ethyl acetate 100/0 to 20/80 as eluent to afford (2R)-4-[3-[4-[5-[tert-butyl(dimethyl)silyl]oxy-1-tetrahydropyran-2-yl-indazol-3-yl]pyrazol-1-yl]propoxy]butan-2-ol as a colorless oil.

LCMS method F: [M+H]⁺=529.3, t_{R}=3.34 min

### Preparation of Intermediate 195: [(1R)-3-[3-[4-[5-[tert-butyl(dimethyl)silyl]oxy-1-tetrahydropyran-2-yl-indazol-3-yl]pyrazol-1-yl]propoxy]-1-methyl-propyl]methane sulfonate

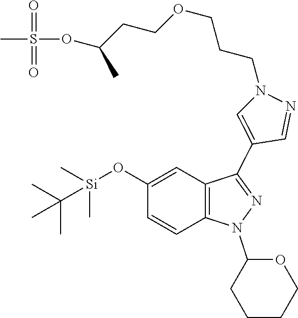 [SMILES: C[C@H](CCOCCCn1cc(-c2nn(C3CCCCO3)c3ccc(O[Si](C)(C)C(C)(C)C)cc23)cn1)OS(C)(=O)=O]

To a solution of (2R)-4-[3-[4-[5-[tert-butyl(dimethyl)silyl]oxy-1-tetrahydropyran-2-yl-indazol-3-yl]pyrazol-1-yl]propoxy]butan-2-ol (210 mg, 0.40 mmol) and triethylamine (111 μL, 0.79 mmol) in dichloromethane (4 mL) at 0° C. was added methanesulfonyl chloride (40 μL, 0.52 mmol) in dichloromethane (1 mL). The reaction mixture was stirred at RT for 2 h. The residue was diluted with brine and the aqueous layer was extracted with dichloromethane. The combined organic layers were dried over anhydrous sodium sulfate, filtered and evaporated under reduced pressure to afford [(1R)-3-[3-[4-[5-[tert-butyl(dimethyl)silyl]oxy-1-tetrahydro pyran-2-yl-indazol-3-yl]pyrazol-1-yl]propoxy]-1-methyl-propyl]methanesulfonate as a yellow oil which was used in the next step without further purification.

LCMS method F: [M+H]⁺=607.3, t_{R}=3.46 min

### Preparation of Intermediate 196: [(1R)-3-[3-[4-(5-hydroxy-1-tetrahydropyran-2-yl-indazol-3-yl)pyrazol-1-yl]propoxy]-1-methyl-propyl]methanesulfonate

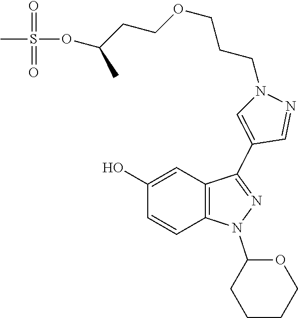 [SMILES: C[C@H](CCOCCCn1cc(-c2nn(C3CCCCO3)c3ccc(O)cc23)cn1)OS(C)(=O)=O]

To a solution of [(1R)-3-[3-[4-[5-[tert-butyl(dimethyl)silyl]oxy-1-tetrahydropyran-2-yl-indazol-3-yl]pyrazol-1-yl]propoxy]-1-methyl-propyl] methanesulfonate (241 mg, 0.40 mmol) in THF (5 mL) at −40° C. was added TBAF (1M solution in THF) (437 □L, 0.44 mmol). The reaction mixture was stirred at −40° C. for 15 min. The reaction mixture was quenched at −40° C. with saturated aqueous ammonium chloride solution and the aqueous layer was extracted with ethyl acetate. The combined organic layers were dried over anhydrous sodium sulfate, filtered and evaporated under reduced pressure affording [(1R)-3-[3-[4-(5-hydroxy-1-tetrahydropyran-2-yl-indazol-3-yl)pyrazol-1-yl]propoxy]-1-methyl-propyl]methanesulfonate as a yellow oil which was used in the next step without further purification.

LCMS method F: [M+H]⁺=493.2, t_{R}=2.38 min

### Preparation of Intermediate 197: (12S)-12-methyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene

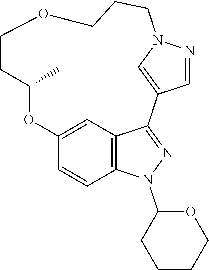 [SMILES: C[C@H]1CCOCCCn2cc(cn2)-c2nn(C3CCCCO3)c3ccc(cc23)O1]

To a suspension of cesium carbonate (195 mg, 0.60 mmol) in anhydrous DMF (80 mL) at 80° C. was added dropwise [(1R)-3-[3-[4-(5-hydroxy-1-tetrahydropyran-2-yl-indazol-3-yl)pyrazol-1-yl]propoxy]-1-methyl-propyl] methanesulfonate (98 mg, 0.20 mmol) in DMF (80 mL). The reaction mixture was stirred at 80° C. for 1 h. The solvent was evaporated under reduced pressure. The residue was diluted with brine and extracted with ethyl acetate. The combined organic layers were dried over anhydrous sodium sulfate, filtered and evaporated under reduced pressure. The residue was purified by silica gel column chromatography using cyclohexane/(ethyl acetate/ethanol 3/1)) 100/0 to 80/20 as eluent to afford (12S)-12-methyl-18-(oxan-2-yl)-9,13-dioxa-4,5,18,19-tetraazatetracyclo[12.5.2.1^{2,5}.0^{17,20}]docosa-1(19),2(22),3, 14(21),15,17(20)-hexaene as a colorless oil.

LCMS method J: [M+H]⁺=397.2, t_{R}=4.01 min

### Preparation of Example 32: (12S)-12-methyl-9,13-dioxa-4,5,18,19-tetraazatetracyclo [12.5.2.1^{2,5}.0^{17,20}]docosa-1(19),2(22),3,14(21),15,17(20)-hexaene

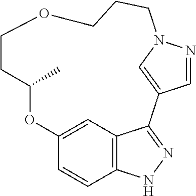 [SMILES: C[C@H]1CCOCCCn2cc(cn2)-c2n[nH]c3ccc(cc23)O1]

To a solution of (12S)-12-methyl-18-(oxan-2-yl)-9,13-dioxa-4,5,18,19-tetraaza tetracyclo [12.5.2.1^{2,5}.0^{17,20}]docosa-1(19),2(22),3,14(21),15,17(20)-hexaene (20 mg, 0.05 mmol) in methanol (3.5 mL) and water (0.5 mL) was added p-toluenesulfonic acid monohydrate (48 mg, 0.25 mmol). The reaction mixture was stirred at 65° C. for 2 h. The solvent was evaporated under reduced pressure and the residue was neutralized by addition of a saturated aqueous NaHCO₃ solution. ethyl acetate was added, the layers were separated and the aqueous phase was extracted with ethyl acetate. The combined organic layers were washed with brine, dried over anhydrous sodium sulfate, filtered and evaporated under reduced pressure. The residue was purified by silica gel column chromatography using dichloromethane/methanol 100/0 to 98/2 as eluent. The resulting solid was crystallized in diisopropylether, filtered and dried to afford (12S)-12-methyl-9,13-dioxa-4,5,18,19-tetraazatetracyclo[12.5.2.1^{2,5}.0^{17,20}]docosa-1(19),2(22), 3,14(21),15,17(20)-hexaene as a solid.

LCMS method F: [M+H]⁺=313.3, t_{R}=2.12 min

LCMS method G: [M+H]⁺=313.3, t_{R}=2.12 min

¹H NMR (400 MHz, d6-DMSO) 12.67 (1H, s), 8.63 (1H, s), 7.67 (1H, s), 7.46 (1H, d, J=2.1 Hz), 7.40-7.37 (1H, m), 6.93 (1H, dd, J=2.3, 8.9 Hz), 4.55-4.42 (2H, m), 4.31 (1H, ddd, J=1.5, 8.8, 14.3 Hz), 3.75-3.51 (4H, m), 2.49-2.39 (1H, m), 2.24-2.05 (2H, m), 1.56-1.45 (1H, m), 1.41 (3H, d) ppm.

### Example 33: (8R,13S)-8,13-dimethyl-7,10,14-trioxa-19,20,23-triazatetracyclo[13.5.2.1^{2,6}.0^{18,21}]tricosa-1(20),2(23),3,5,15(22),16,18(21)-heptaene

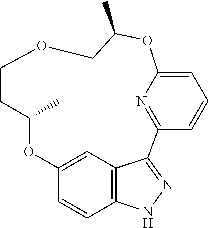 [SMILES: C[C@@H]1COCC[C@H](C)Oc2ccc3[nH]nc(c3c2)-c2cccc(n2)O1]

Example 33 is prepared according to the synthesis route described in general Scheme D.

### Preparation of Intermediate 198: 2-[(1R)-2-[(3R)-3-benzyloxybutoxy]-1-methyl-ethoxy]-6-bromo-pyridine

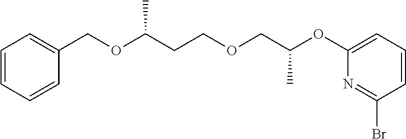 [SMILES: C[C@H](CCOC[C@@H](C)Oc1cccc(Br)n1)OCc1ccccc1]

(2S)-1-[(3R)-3-benzyloxybutoxy]propan-2-ol was prepared according to the same synthesis procedures as (2R)-1-[(3R)-3-benzyloxybutoxy]propan-2-ol (intermediate 133) starting from 2-[(1R)-2-[(3R)-3-benzyloxybutoxy]-1-methyl-ethoxy]tetrahydropyran.

To a solution of 6-bromopyridin-2-ol (150 mg, 0.86 mmol), (2S)-1-[(3R)-3-benzyloxybutoxy] propan-2-ol (267 mg, 1.12 mmol) and triphenylphosphine (339 mg, 1.29 mmol) in dry THF (15 mL), was added under argon atmosphere DIAD (255 μL, 1.29 mmol). The reaction mixture was stirred at RT overnight. The solvent was removed under reduced pressure. The residue was purified by silica gel column chromatography using cyclohexane/ethyl acetate 100/0 to 70/30 as eluent to afford 2-[(1R)-2-[(3R)-3-benzyloxybutoxy]-1-methyl-ethoxy]-6-bromo-pyridine as a colorless oil.

LCMS method F: [M+H]⁺=396.1, t_{R}=3.37 min

### Preparation of Intermediate 199: [3-[6-[(1R)-2-[(3R)-3-benzyloxybutoxy]-1-methyl-ethoxy]-2-pyridyl]-1-tetrahydropyran-2-yl-indazol-5-yl]oxy-tert-butyl-dimethyl-silane

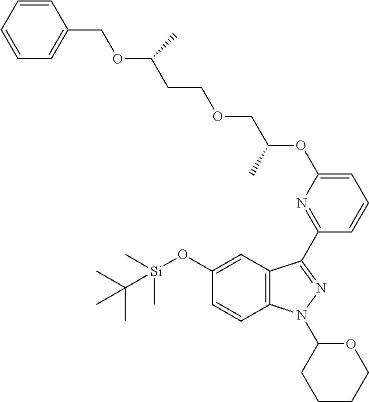 [SMILES: C[C@H](CCOC[C@@H](C)Oc1cccc(-c2nn(C3CCCCO3)c3ccc(O[Si](C)(C)C(C)(C)C)cc23)n1)OCc1ccccc1]

To a degassed solution of tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (560 mg, 1.22 mmol), 2-[(1R)-2-[(3R)-3-benzyloxybutoxy]-1-methyl-ethoxy]-6-bromo-pyridine (400 mg, 1.017 mmol), potassium phosphate tribasic (648 mg, 3.051 mmol) in dioxane (30 mL) and water (3 mL) was added tetrakis(triphenylphosphine)palladium(0) (59 mg, 0.051 mmol). The reaction mixture was stirred at 110° C. for 2 h. The reaction mixture was filtered through a pad of Celite and washed with ethyl acetate. The filtrate was diluted with water and the aqueous layer was extracted with ethyl acetate. The combined organic layers were washed with water and brine, dried over anhydrous sodium sulfate and evaporated under reduced pressure. The residue was purified by silica gel column chromatography using cyclohexane/ethyl acetate 100/0 to 90/10 as eluent to afford [3-[6-[(1R)-2-[(3R)-3-benzyloxybutoxy]-1-methyl-ethoxy]-2-pyridyl]-1-tetrahydropyran-2-yl-indazol-5-yl]oxy-tert-butyl-dimethyl-silane as a colorless oil.

LCMS method M: [M+H]⁺=646.4, t_{R}=6.19 min

### Preparation of Intermediate 200: (2R)-4-[(2R)-2-[[6-[5-[tert-butyl(dimethyl)silyl] oxy-1-tetrahydropyran-2-yl-indazol-3-yl]-2-pyridyl]oxy]propoxy]butan-2-ol

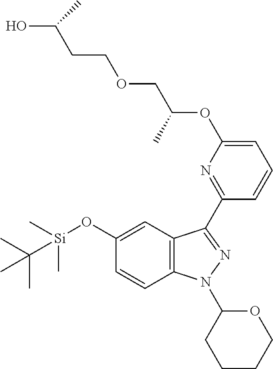 [SMILES: C[C@H](COCC[C@@H](C)O)Oc1cccc(-c2nn(C3CCCCO3)c3ccc(O[Si](C)(C)C(C)(C)C)cc23)n1]

To a solution of [3-[6-[(1R)-2-[(3R)-3-benzyloxybutoxy]-1-methyl-ethoxy]-2-pyridyl]-1-tetrahydropyran-2-yl-indazol-5-yl]oxy-tert-butyl-dimethyl-silane (510 mg, 0.79 mmol) in ethanol (20 mL) at RT was added Pd/C 10% (50 mg). The reaction mixture was stirred under hydrogen atmosphere at 60° C. for 3 h. The reaction mixture was filtered and the solvent was removed under reduced pressure to afford (2R)-4-[(2R)-2-[[6-[5-[tert-butyl(dimethyl)silyl] oxy-1-tetrahydropyran-2-yl-indazol-3-yl]-2-pyridyl]oxy]propoxy]butan-2-ol as a colorless oil which was used in the next step without further purification.

LCMS method M: [M+H]⁺=556.3, t_{R}=4.89 min

### Preparation of Intermediate 201: [(1R)-3-[(2R)-2-[[6-[5-[tert-butyl(dimethyl)silyl] oxy-1-tetrahydropyran-2-yl-indazol-3-yl]-2-pyridyl]oxy]propoxy]-1-methyl-propyl] methanesulfonate

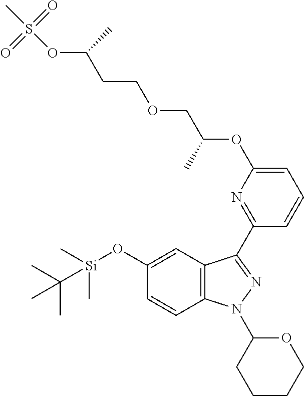 [SMILES: C[C@H](COCC[C@@H](C)OS(C)(=O)=O)Oc1cccc(-c2nn(C3CCCCO3)c3ccc(O[Si](C)(C)C(C)(C)C)cc23)n1]

To a solution of (2R)-4-[(2R)-2-[[6-[5-[tert-butyl(dimethyl)silyl]oxy-1-tetrahydropyran-2-yl-indazol-3-yl]-2-pyridyl]oxy]propoxy]butan-2-ol (330 mg, 0.59 mmol) and triethylamine (166 μL, 1.19 mmol) in dichloromethane (10 mL) at 0° C. was added methanesulfonyl chloride (60 μL, 0.77 mmol) in dichloromethane (5 mL). The reaction mixture was stirred at RT for 1 h. The residue was diluted with water and the aqueous layer was extracted with dichloromethane. The combined organic layers were dried over anhydrous sodium sulfate, filtered and evaporated under reduced pressure to afford [(1R)-3-[(2R)-2-[[6-[5-[tert-butyl(dimethyl)silyl]oxy-1-tetrahydropyran-2-yl-indazol-3-yl]-2-pyridyl]oxy]propoxy]-1-methyl-propyl]methane sulfonate as a colorless oil which was used in the next step without further purification.

LCMS method M: [M+H]⁺=634.5, t_{R}=5.17-5.28 min

### Preparation of Intermediate 202: (8R,13S)-8,13-dimethyl-19-(oxan-2-yl)-7,10,14-trioxa-19,20,23-triazatetracyclo[13.5.2.1^{2,6}.0^{18,21}]tricosa-1(20),2(23),3,5,15(22),16, 18(21)-heptaene

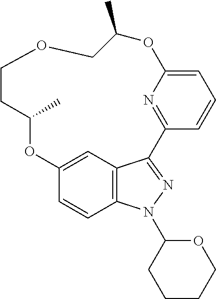 [SMILES: C[C@@H]1COCC[C@H](C)Oc2ccc3c(c2)c(nn3C2CCCCO2)-c2cccc(n2)O1]

To a suspension of sodium hydride (60% dispersion in mineral oil) (0.076 g, 1.89 mmol) in anhydrous DMF (200 mL) at RT was added dropwise [(1R)-3-[(2R)-2-[[6-[5-[tert-butyl (dimethyl)silyl]oxy-1-tetrahydropyran-2-yl-indazol-3-yl]-2-pyridyl]oxy]propoxy]-1-methyl-propyl]methanesulfonate (400 mg, 0.63 mmol) in DMF (200 mL). The reaction mixture was stirred at RT overnight. The solvent was removed under reduced pressure and the resulting solid was triturated with water. The solid was filtered, washed with water and dried under reduced pressure to afford (8R,13S)-8,13-dimethyl-19-(oxan-2-yl)-7,10,14-trioxa-19,20,23-triazatetra cyclo[13.5.2.1^{2,6}.0^{18,21}]tricosa-1(20),2(23),3,5,15(22),16,18(21)-heptaene as a pink powder.

LCMS method F: [M+H]⁺=424.2, t_{R}=3.67 min

### Preparation of Example 33: (8R,13S)-8,13-dimethyl-7,10,14-trioxa-19,20,23-triaza tetracyclo[13.5.2.1^{2,6}.0^{18,21}]tricosa-1(20),2(23),3,5,15(22),16,18(21)-heptaene

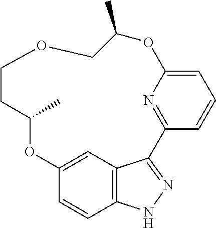 [SMILES: C[C@@H]1COCC[C@H](C)Oc2ccc3[nH]nc(c3c2)-c2cccc(n2)O1]

To a solution of (8R,13S)-8,13-dimethyl-19-(oxan-2-yl)-7,10,14-trioxa-19,20,23-triazatetra cyclo[13.5.2.1^{2,6}.0^{18,21}]tricosa-1(20),2(23),3,5,15(22),16,18(21)-heptaene (226 mg, 0.53 mmol) in methanol (25 mL) and water (3 mL) was added p-toluenesulfonic acid monohydrate (508 mg, 2.67 mmol). The reaction mixture was stirred at 65° C. for 2 h. The solvent was evaporated under reduced pressure and the residue was neutralized by addition of a saturated aqueous NaHCO₃ solution. ethyl acetate was added, the layers were separated and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent. The resulting solid was crystallized from diisopropylether to afford (8R,13S)-8,13-dimethyl-7,10,14-trioxa-19,20,23-triazatetracyclo[13.5.2.1^{2,6}.0^{18,21}]tricosa-1(20),2(23),3,5, 15(22),16,18(21)-heptaene as a powder.

LCMS method F: [M+H]⁺=340.3, t_{R}=3.04 min

LCMS method G: [M+H]⁺=340.3, t_{R}=3.01 min

¹H NMR (400 MHz, d6-DMSO) 13.25-13.22 (1H, m), 8.07 (1H, d, J=2.3 Hz), 7.78-7.76 (2H, m), 7.49-7.45 (1H, m), 7.00 (1H, dd, J=2.3, 8.9 Hz), 6.73-6.70 (1H, m), 5.27-5.22 (1H, m), 4.63 (1H, dd, J=6.1, 12.5 Hz), 4.25 (1H, dd, J=3.7, 7.5 Hz), 3.79-3.72 (1H, m), 3.63-3.58 (1H, m), 3.34 (1H, s), 2.35-2.28 (1H, m), 1.46-1.35 (7H, m) ppm.

### Example 34: (13S)-4-(3-methoxyazetidin-1-yl)-13-methyl-19-(oxan-2-yl)-7,10-dioxa-5,14, 19,20,23-pentaazatetracyclo[13.5.2.1^{2,6}.0^{18,21}]tricosa-1(20),2,4,6(23),15(22), 16,18(21)-heptaene

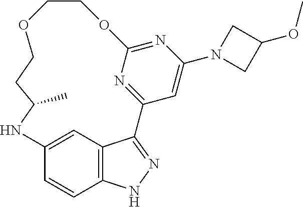 [SMILES: COC1CN(c2cc3nc(n2)OCCOCC[C@H](C)Nc2ccc4[nH]nc-3c4c2)C1]

Example 34 is prepared according to the synthesis route described in general Scheme E, following the same synthesis procedures as for Example 30.

The Suzuki reaction was carried out on 5-((tert-butyldimethylsilyl)oxy)-1-(tetrahydro-2H-pyran-2-yl)-3-(4,4,5,5-tetramethyl-1,3,2-dioxaborolan-2-yl)-1H-indazole with 4,6-dichloro-2-(methylthio)pyrimidine to afford 5-((tert-butyldimethylsilyl)oxy)-3-(6-chloro-2-(methylthio) pyrimidin-4-yl)-1-(tetrahydro-2H-pyran-2-yl)-1H-indazole, which was then reacted with 3-methoxyazetidine hydrochloride yielding 5-((tert-butyldimethylsilyl)oxy)-3-(6-(3-methoxy azetidin-1-yl)-2-(methylthio)pyrimidin-4-yl)-1-(tetrahydro-2H-pyran-2-yl)-1H-indazole. The next steps were carried out according to the same experimental procedures as for example 30.

### Preparation of Intermediate 203: (13S)-4-(3-methoxyazetidin-1-yl)-13-methyl-14-(2-nitrobenzenesulfonyl)-19-(oxan-2-yl)-7,10-dioxa-5,14,19,20,23-pentaazatetracyclo [13.5.2.1^{2,6}.0^{18,21}]tricosa-1(20),2(23),3,5,15(22),16,18(21)-heptaene

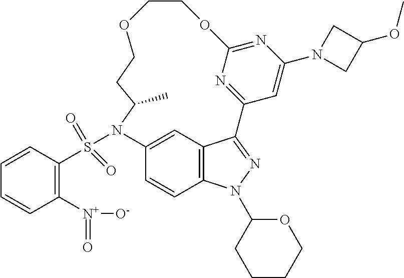 [SMILES: COC1CN(c2cc3nc(n2)OCCOCC[C@H](C)N(S(=O)(=O)c2ccccc2[N+](=O)[O-])c2ccc4c(c2)c-3nn4C2CCCCO2)C1]

A solution of N—((R)-4-(2-hydroxyethoxy)butan-2-yl)-N-(3-(6-(3-methoxyazetidin-1-yl)-2-(methylsulfonyl)pyrimidin-4-yl)-1-(tetrahydro-2H-pyran-2-yl)-1H-indazol-5-yl)-2-nitro benzenesulfonamide (117 mg, 0.154 mmol) in dry THF (8 mL) was added dropwise under nitrogen atmosphere to a stirred solution of sodium hydride (60% dispersion in mineral oil) (2 mg, 0.045 mmol) in dry THF (8 mL) at 60° C. The reaction mixture was stirred at 60° C. for 1.5 h. The reaction mixture was cooled at 0° C. diluted with ethyl acetate and quenched with water. The aqueous layer was extracted with ethyl acetate. The combined organic layers were dried over anhydrous magnesium sulfate, filtered and evaporated under reduced pressure. The residue was purified by silica gel column chromatography using heptane/ethyl acetate 100/0 to 25/75 as eluent to afford (13S)-4-(3-methoxyazetidin-1-yl)-13-methyl-14-(2-nitrobenzenesulfonyl)-19-(oxan-2-yl)-7,10-dioxa-5,14,19,20,23-pentaazatetracyclo[13.5.2.1^{2,6}.0^{18,21}]tricosa-1(20),2(23),3,5,15(22),16,18(21)-heptaene as a colorless oil.

LCMS method C: [M+H]⁺=680.4, t_{R}=4.68 and 4.76 min

### Preparation of Intermediate 204: (13S)-4-(3-methoxyazetidin-1-yl)-13-methyl-19-(oxan-2-yl)-7,10-dioxa-5,14,19,20,23-pentaazatetracyclo[13.5.2.1^{2,6}.0^{18,21}]tricosa-1(20),2,4,6(23),15(22),16,18(21)-heptaene

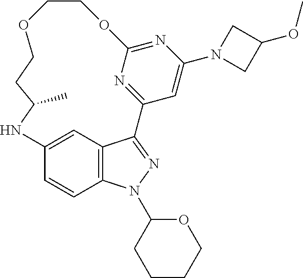 [SMILES: COC1CN(c2cc3nc(n2)OCCOCC[C@H](C)Nc2ccc4c(c2)c-3nn4C2CCCCO2)C1]

To a suspension of (13S)-4-(3-methoxyazetidin-1-yl)-13-methyl-14-(2-nitrobenzenesulfonyl)-19-(oxan-2-yl)-7,10-dioxa-5,14,19,20,23-pentaazatetracyclo[13.5.2.1^{2,6}.0^{18,21}]tricosa-1(20), 2(23),3,5,15(22),16,18(21)-heptaene (90 mg, 0.132 mmol) and cesium carbonate (129 mg, 0.396 mmol) in DMF (1.5 mL) at 0° C. was added thiophenol (40 μL, 0.396 mmol). The reaction mixture was stirred at RT for 2 h. The reaction mixture was diluted with ethyl acetate and 1N aqueous sodium hydroxide solution and the aqueous layer was extracted with ethyl acetate. The combined organic layers were dried over anhydrous magnesium sulfate, filtered and evaporated under reduced pressure. The residue was purified by silica gel column chromatography using heptane/ethyl acetate 100/0 to 40/60 as eluent to afford (13S)-4-(3-methoxyazetidin-1-yl)-13-methyl-19-(oxan-2-yl)-7,10-dioxa-5,14,19,20,23-pentaazatetracyclo[13.5.2.1^{2,6}.0^{18,21}]tricosa-1(20),2,4,6(23),15(22),16,18(21)-heptaene as a yellow oil.

LCMS method B: [M+H]⁺=495.2, t_{R}=1.044 min

### Preparation of Example 34: (13S)-4-(3-methoxyazetidin-1-yl)-13-methyl-7,10-dioxa-5,14,19,20,23-pentaazatetracyclo[13.5.2.1^{2,6}.0^{18,21}]tricosa-1(20),2,4,6(23),15(22),16, 18(21)-heptaene

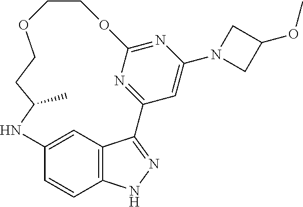 [SMILES: COC1CN(c2cc3nc(n2)OCCOCC[C@H](C)Nc2ccc4[nH]nc-3c4c2)C1]

To a solution of (13S)-4-(3-methoxyazetidin-1-yl)-13-methyl-19-(oxan-2-yl)-7,10-dioxa-5,14,19,20,23-pentaazatetracyclo[13.5.2.1^{2,6}.0^{18,21}]tricosa-1(20),2,4,6(23),15(22),16,18(21)-heptaene (48 mg, 0.097 mmol) in dichloromethane (0.75 mL) at 0° C. was added TFA (0.75 mL). The reaction mixture was stirred at RT for 5 h. The solvent was evaporated under reduced pressure and co-evaporated with methanol. The resulting solid was diluted with dichloromethane, cooled at 0° C. and neutralized with a saturated aqueous NaHCO₃ solution. The aqueous layer was extracted with dichloromethane. The combined organic layers were dried over anhydrous magnesium sulfate, filtered and evaporated under reduced pressure. The residue was purified by silica gel column chromatography using dichloromethane/ethyl acetate 100/0 to 70/30 as eluent. The desired fractions were combined and the solvent was removed under reduced pressure to afford (13S)-4-(3-methoxyazetidin-1-yl)-13-methyl-7,10-dioxa-5,14,19,20,23-pentaazatetracyclo[13.5.2.1^{2,6}.0^{18,21}]tricosa-1(20),2,4,6(23),15(22),16,18(21)-heptaene as a solid.

LCMS method E: [M+H]⁺=411.1, t_{R}=2.409 min

LCMS method D: [M+H]⁺=411.1, t_{R}=3.355 min

¹H NMR (400 MHz, d6-DMSO) 13.12 (s, 1H), 7.53 (d, J=2.1 Hz, 1H), 7.31 (d, J=8.9 Hz, 1H), 6.85 (dd, J=9.0, 2.1 Hz, 1H), 6.68 (s, 1H), 5.41 (d, J=6.5 Hz, 1H), 4.89 (td, J=10.4, 5.3 Hz, 1H), 4.35 (tt, J=6.2, 3.9 Hz, 1H), 4.30-4.17 (m, 3H), 4.10 (td, J=10.6, 5.1 Hz, 1H), 3.85 (dd, J=9.7, 3.8 Hz, 2H), 3.72 (td, J=11.9, 2.5 Hz, 1H), 3.69-3.55 (m, 1H), 3.54-3.41 (m, 2H), 3.26 (s, 3H), 2.47-2.38 (m, 1H), 1.24 (d, J=6.5 Hz, 3H), 1.01-0.89 (m, 1H) ppm.

### Example 35: (6R,12R)-6,12-dimethyl-9,13-dioxa-4,5,18,19-tetraazatetracyclo[12.5.2.1^{2,5}.0^{17,20}]docosa-1(19),2(22),3,14(21),15,17(20)-hexaene

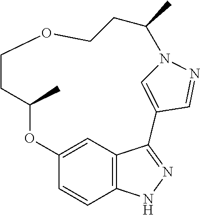 [SMILES: C[C@@H]1CCOCC[C@@H](C)n2cc(cn2)-c2n[nH]c3ccc(cc23)O1]

Example 35 is prepared according to the synthesis route described in general Scheme D, following the same synthesis procedures as for Example 25.

### Preparation of Intermediate 205: (6R,12R)-6,12-dimethyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene

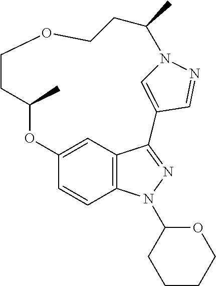 [SMILES: C[C@@H]1CCOCC[C@@H](C)n2cc(cn2)-c2nn(C3CCCCO3)c3ccc(cc23)O1]

To a heated suspension of cesium carbonate (779 mg, 2.392 mmol) in dry DMF (210 mL) at 90° C. was added dropwise [(1S)-3-[(3R)-3-[4-[5-[tert-butyl(dimethyl)silyl]oxy-1-tetrahydro pyran-2-yl-indazol-3-yl]pyrazol-1-yl]butoxy]-1-methyl-propyl]methanesulfonate (371 mg, 0.598 mmol) in dry DMF (200 mL). The reaction mixture was stirred at 90° C. for 1 h. The solvent was evaporated under reduced pressure and diluted with brine and ethyl acetate. The aqueous layer was extracted with ethyl acetate. The combined organic layers were dried over anhydrous sodium sulfate, filtered and evaporated under reduced pressure. The residue was firstly purified by silica gel column chromatography using dichloromethane/methanol 100/0 to 95/5 as eluent. The resulting product was further purified by preparative-TLC eluting with dichloromethane/methanol 95/5 to afford (6R,12R)-6,12-dimethyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene as a pale yellow oil.

LCMS method L: [M+H]⁺=411, t_{R}=2.81 min

### Preparation of Example 35: (6R,12R)-6,12-dimethyl-9,13-dioxa-4,5,18,19-tetraaza tetracyclo[12.5.2.1^{2,5}.0^{17,20}]docosa-1(19),2(22),3,14(21),15,17(20)-hexaene

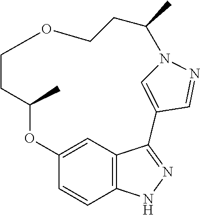 [SMILES: C[C@@H]1CCOCC[C@@H](C)n2cc(cn2)-c2n[nH]c3ccc(cc23)O1]

To a solution of (6R,12R)-6,12-dimethyl-18-(oxan-2-yl)-9,13-dioxa-4,5,18,19-tetraazatetra cyclo[12.5.2.1^{2,5}.0^{17,20}]docosa-1(19),2(22),3,14(21),15,17(20)-hexaene (52 mg, 0.127 mmol) in methanol (7.5 mL) and water (1 mL) was added p-toluenesulfonic acid monohydrate (121 mg, 0.635 mmol). The reaction mixture was stirred at 65° C. for 5 h. The solvent was evaporated under reduced pressure and the residue was neutralized by slow addition of a saturated aqueous NaHCO₃ solution. ethyl acetate was added and the aqueous layer was extracted with ethyl acetate. The combined organic layers were washed with brine, dried over anhydrous sodium sulfate, filtered and the solvent was removed under reduced pressure. The residue was purified by silica gel column chromatography using cyclohexane/(ethyl acetate/ethanol (3/1)) 100/0 to 50/50 as eluent. The resulting solid was triturated from diisopropyl ether, filtered and dried to afford (6R,12R)-6,12-dimethyl-9,13-dioxa-4,5,18,19-tetraazatetracyclo[12.5.2.1^{2,5}.0^{17,20}] docosa-1(19),2(22),3,14(21),15,17(20)-hexaene as a solid.

LCMS method L: [M+H]⁺=327.3, t_{R}=2.23 min

LCMS method G: [M+H]⁺=327.3, t_{R}=2.22 min

¹H NMR (400 MHz, CDCl₃) δ 8.52 (1H, s), 7.97 (1H, s), 7.60 (1H, m), 7.42 (1H, d, J=9.08 Hz), 7.09 (1H, dd, J=2.0, 9.0 Hz), 4.68-4.55 (2H, m), 3.80-3.75 (1H, m), 3.71-3.61 (2H, m), 3.58-3.52 (1H, m), 2.58-2.49 (1H, m), 2.43-2.25 (2H, m), 1.67 (3H, d, J=6.78 Hz), 1.61-1.52 (1H, m), 1.49 (3H, d, J=6.12 Hz) ppm.

### Example 36: (6S,12R)-6,12-dimethyl-9,13-dioxa-4,5,18,19-tetraazatetracyclo[12.5.2.1^{2,5}.0^{17,20}]docosa-1(19),2(22),3,14(21),15,17(20)-hexaene

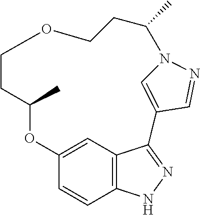 [SMILES: C[C@@H]1CCOCC[C@H](C)n2cc(cn2)-c2n[nH]c3ccc(cc23)O1]

Example 36 is prepared according to the synthesis route described in general Scheme D, following the same synthesis procedures as for Example 25.

### Preparation of Intermediate 206: (6R,12R)-6,12-dimethyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene

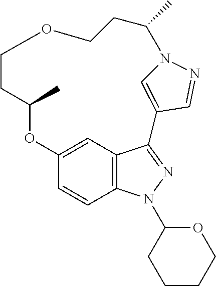 [SMILES: C[C@@H]1CCOCC[C@H](C)n2cc(cn2)-c2nn(C3CCCCO3)c3ccc(cc23)O1]

To a suspension of cesium carbonate (779 mg, 2.392 mmol) in dry DMF (210 mL) at 90° C. was added dropwise [(1S)-3-[(3R)-3-[4-[5-[tert-butyl(dimethyl)silyl]oxy-1-tetrahydropyran-2-yl-indazol-3-yl]pyrazol-1-yl]butoxy]-1-methyl-propyl]methanesulfonate (371 mg, 0.598 mmol) in dry DMF (200 mL). The reaction mixture was stirred at 90° C. for 1 h. The reaction mixture was concentrated under reduced pressure and diluted with brine and ethyl acetate. The aqueous layer was extracted with ethyl acetate. The combined organic layers were dried over anhydrous sodium sulfate, filtered and evaporated under reduced pressure. The residue was purified by silica gel column chromatography using dichloromethane/methanol 100/0 to 95/5 as eluent. The resulting product was purified preparative TLC eluting with dichloromethane/methanol 95/5 to afford (6R,12R)-6,12-dimethyl-18-(oxan-2-yl)-9,13-dioxa-4,5,18,19-tetraazatetracyclo [12.5.2.1^{2,5}.0^{17,20}]docosa-1(19),2(22),3,14(21),15,17(20)-hexaene as a pale yellow oil.

LCMS method F: [M+H]⁺=411.4, t_{R}=2.82 min

### Preparation of Example 36: (6S,12R)-6,12-dimethyl-9,13-dioxa-4,5,18,19-tetraaza tetracyclo[12.5.2.1^{2,5}.0^{17,20}]docosa-1(19),2(22),3,14(21),15,17(20)-hexaene

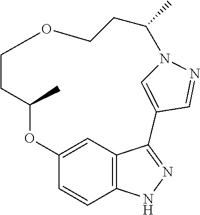 [SMILES: C[C@@H]1CCOCC[C@H](C)n2cc(cn2)-c2n[nH]c3ccc(cc23)O1]

To a solution of (6S,12R)-6,12-dimethyl-18-(oxan-2-yl)-9,13-dioxa-4,5,18,19-tetraazatetra cyclo[12.5.2.1^{2,5}.0^{17,20}]docosa-1(19),2(22),3,14(21),15,17(20)-hexaene (60 mg, 0.146 mmol) in methanol (8.6 mL) and water (1.1 mL) was added p-toluenesulfonic acid monohydrate (139 mg, 0.730 mmol). The reaction mixture was stirred at 65° C. for 5 h. The solvent was evaporated under reduced pressure and the residue was neutralized by slow addition of a saturated aqueous NaHCO₃ solution. ethyl acetate was added and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/(ethyl acetate/ethanol (3/1)) 100/0 to 50/50 as eluent. The resulting product was then purified by preparative-TLC eluting with cyclohexane/(ethyl acetate/ethanol (3-1)) 50/50 to afford (6S,12R)-6,12-dimethyl-9,13-dioxa-4,5,18,19-tetraazatetracyclo[12.5.2.1^{2,5}.0^{17,20}]docosa-1(19),2(22),3,14(21),15,17(20)-hexaene as a solid.

LCMS method L: [M+H]⁺=327.3, t_{R}=2.24 min

LCMS method G: [M+H]⁺=327.3, t_{R}=2.23 min

¹H NMR (400 MHz, CDCl3) δ 8.66 (1H, s), 7.92 (1H, s), 7.57 (1H, d), 7.39-7.37 (1H, d, J=9.0 Hz), 7.08-7.04 (1H, dd, J=2.3, 8.9 Hz), 4.83-4.75 (1H, m), 4.65-4.57 (1H, m), 3.80-3.75 (1H, m), 3.71-3.64 (1H, m), 3.62-3.55 (2H, m), 2.64-2.52 (1H, m), 2.42-2.32 (1H, m), 2.21-2.11 (1H, m), 1.66 (3H, d, J=6.8 Hz), 1.64-1.57 (1H, m), 1.49 (3H, d, J=6 Hz) ppm.

### Example 37: (8S)-8-Methyl-9,13-dioxa-4,5,18,19-tetraazatetracyclo[12.5.2.1^{2,5}.0^{17,20}] docosa-1(19),2(22),3,14(21),15,17(20)-hexaene

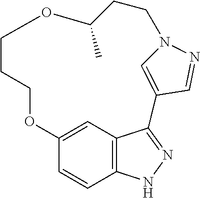 [SMILES: C[C@H]1CCn2cc(cn2)-c2n[nH]c3ccc(cc23)OCCCO1]

Example 37 is prepared according to the synthesis route described in general Scheme D.

### Preparation of Intermediate 207: 3-(benzyloxy)propyl methanesulfonate

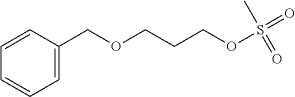 [SMILES: CS(=O)(=O)OCCCOCc1ccccc1]

To a solution of 3-benzyloxy-1-propanol (4 g, 24.064 mmol) in dichloromethane (73 mL) under nitrogen at 0° C. was added triethylamine (5.031 mL, 36.096 mmol) and methanesulfonyl chloride (2.421 mL, 31.283 mmol). The reaction mixture was stirred for 2 h at RT. The reaction mixture was diluted with dichloromethane and washed with a saturated aqueous NaHCO₃ solution. The aqueous phase was extracted with dichloromethane. The combined organic layers were washed with brine, dried over anhydrous magnesium sulfate, filtered and evaporated under reduced pressure to afford 3-(benzyloxy)propylmethanesulfonate as a yellow oil which was used in the next step without further purification.

LCMS method B: [M+H]+=244.9, tR=0.701 min

### Preparation of Intermediate 208: [(3S)-3-(3-benzyloxypropoxy)butoxy]-tert-butyl-diphenyl-silane

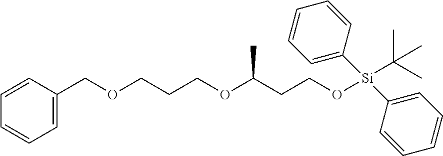 [SMILES: C[C@@H](CCO[Si](c1ccccc1)(c1ccccc1)C(C)(C)C)OCCCOCc1ccccc1]

To a solution of (S)-4-((tert-butyldiphenylsilyl)oxy)butan-2-ol (intermediate 153) (6.59 g, 20.059 mmol) in dry THF (100 mL) at 0° C. under nitrogen atmosphere was added sodium hydride (60% dispersion in mineral oil) (2.4 g, 60.177 mmol). The reaction mixture was stirred at 0° C. for 1 h. 3-(Benzyloxy)propyl methanesulfonate (5.881 g, 24.071 mmol) was added and the reaction mixture was stirred at 80° C. for 5 h. The reaction mixture was cooled to 0° C., diluted with ethyl acetate, quenched with a saturated aqueous ammonium chloride solution and washed with brine. The organic layer was dried over anhydrous magnesium sulfate, filtered and evaporated under reduced pressure. The residue was purified by silica gel column chromatography using heptane/ethyl acetate 100/0 to 50/50 as eluent to afford [(3S)-3-(3-benzyloxypropoxy)butoxy]-tert-butyl-diphenyl-silane as a colorless oil.

LCMS method B: [M+H]+=not detected, t_{R}=1.645 min

### Preparation of Intermediate 209: (S)-3-(3-(benzyloxy)propoxy)butan-1-ol

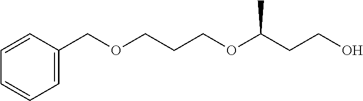 [SMILES: C[C@@H](CCO)OCCCOCc1ccccc1]

To a solution of [(3S)-3-(3-benzyloxypropoxy)butoxy]-tert-butyl-diphenyl-silane (5.1 g, 10.698 mmol) in THF (55 mL) at 0° C. was added TBAF (1M solution in THF) (15.732 mL, 15.732 mmol). The reaction mixture was stirred at RT for 63 h then warmed up to RT. The reaction mixture was diluted with ethyl acetate, washed with a saturated aqueous NaHCO₃ solution and brine. The organic layer was dried over anhydrous magnesium sulfate, filtered and evaporated under reduced pressure. The residue was purified by silica gel column chromatography using a heptane/ethyl acetate 100/0 to 30/70 as eluent to afford (S)-3-(3-(benzyloxy)propoxy)butan-1-ol as a colorless oil.

LCMS method B: [M+H]+=239.1, t_{R}=0.711 min

### Preparation of Intermediate 210: (S)-3-(3-(benzyloxy)propoxy)butylmethane sulfonate

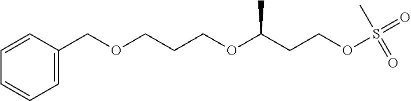 [SMILES: C[C@@H](CCOS(C)(=O)=O)OCCCOCc1ccccc1]

To a solution of (S)-3-(3-(benzyloxy)propoxy)butan-1-ol (1.7 g, 7.133 mmol) in dichloromethane (21.4 mL) at 0° C. under nitrogen atmosphere was added triethylamine (1.491 mL, 10.7 mmol) and methanesulfonyl chloride (718 μL, 9.273 mmol). The reaction mixture was stirred for 2 h then warmed up to RT. The reaction mixture was diluted with dichloromethane and washed with a saturated aqueous NaHCO₃ solutions. The aqueous layer was extracted with dichloromethane and the combined organic layers were washed with brine, dried over magnesium sulfate, filtered and evaporated under reduced pressure to afford (S)-3-(3-(benzyloxy)propoxy)butylmethanesulfonate as a yellow oil which was used in the next step without further purification.

LCMS method B: [M+H]+=317.0, t_{R}=0.875 min

### Preparation of Intermediate 211: 1-[(3S)-3-(3-benzyloxypropoxy)butyl]-4-(4,4,5,5-tetramethyl-1,3,2-dioxaborolan-2-yl)pyrazole

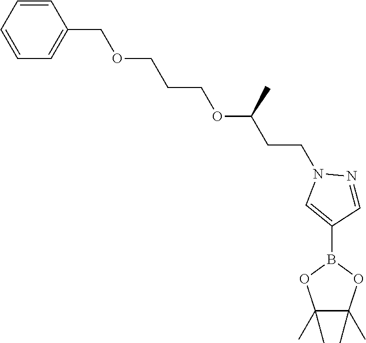 [SMILES: C[C@@H](CCn1cc(B2OC(C)(C)C(C)(C)O2)cn1)OCCCOCc1ccccc1]

To a solution of (S)-3-(3-(benzyloxy)propoxy)butyl methanesulfonate (2.2 g, 6.95 mmol) in dry acetonitrile (21 mL) was added cesium carbonate (4.53 g, 13.90 mmol) and the reaction mixture was stirred at RT for 10 min. 4-(4,4,5,5-tetramethyl-1,3,2-dioxaborolan-2-yl)1H-pyrazole (1.48 g, 7.64 mmol) was added. The reaction mixture was stirred at 90° C. for 15 h. The reaction mixture was diluted with ethyl acetate and washed with brine. The organic layer was dried over anhydrous magnesium sulfate, filtered and evaporated reduced pressure. The residue was purified by silica gel column chromatography using dichloromethane/ethyl acetate 99/1 to 95/5 as eluent to afford 1-[(3S)-3-(3-benzyloxypropoxy)butyl]-4-(4,4,5,5-tetramethyl-1,3,2-dioxaborolan-2-yl)pyrazole as a colorless oil.

LCMS method C: [M+H]⁺=415.3, t_{R}=5.302 min Preparation of intermediates 212: [3-[1-[(3S)-3-(3-benzyloxypropoxy) butyl]pyrazol-4-yl]-1-tetrahydropyran-2-yl-indazol-5-yl]oxy-tert-butyl-dimethyl-silane

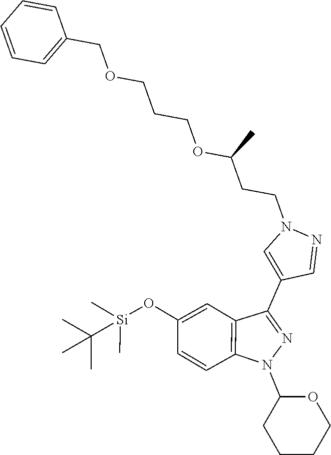 [SMILES: C[C@@H](CCn1cc(-c2nn(C3CCCCO3)c3ccc(O[Si](C)(C)C(C)(C)C)cc23)cn1)OCCCOCc1ccccc1]

To a degassed solution of tert-butyl-(3-iodo-1-tetrahydropyran-2-yl-indazol-5-yl)oxy-dimethyl-silane (780 mg, 1.7 mmol), 1-[(3S)-3-(3-benzyloxypropoxy)butyl]-4-bromo-pyrazole (845 mg, 2.04 mmol), potassium phosphate tribasic (1.08 g, 5.1 mmol) in dioxane (15 mL) and water (2 mL) was added tetrakis(triphenylphosphine)palladium(0) (99 mg, 0.085 mmol). The reaction mixture was stirred at 100° C. for 1 h. The reaction mixture was concentrated under reduced pressure and the resulting oily residue was poured into water and extracted with ethyl acetate. The combined organic layers were washed with water and brine, dried over anhydrous sodium sulfate, filtered and evaporated under reduced pressure. The residue was purified by silica gel column chromatography using cyclohexane/ethyl acetate 100/0 to 50/50 as eluent to afford [3-[1-[(3S)-3-(3-benzyloxypropoxy)butyl]pyrazol-4-yl]-1-tetrahydropyran-2-yl-indazol-5-yl]oxy-tert-butyl-dimethyl-silane as a colorless oil. LCMS method F [M+H]⁺=619.4, t_{R}=3.85 min

### Preparation of Intermediate 213: 3-[(1S)-3-[4-[5-[tert-butyl(dimethyl)silyl]oxy-1-tetrahydropyran-2-yl-indazol-3-yl]pyrazol-1-yl]-1-methyl-propoxy]propan-1-ol

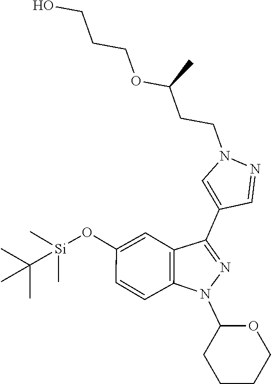 [SMILES: C[C@@H](CCn1cc(-c2nn(C3CCCCO3)c3ccc(O[Si](C)(C)C(C)(C)C)cc23)cn1)OCCCO]

To a solution of [3-[1-[(3S)-3-(3-benzyloxypropoxy)butyl]pyrazol-4-yl]-1-tetrahydropyran-2-yl-indazol-5-yl]oxy-tert-butyl-dimethyl-silane (370 mg, 0.6 mmol) in methanol (20 mL) was added at RT Pd(OH)₂ (50 mg). The reaction mixture was stirred under hydrogen atmosphere at 60° C. for 2.5 h. The reaction mixture was filtered and the solvent was removed under reduced pressure to afford 3-[(1S)-3-[4-[5-[tert-butyl(dimethyl)silyl]oxy-1-tetrahydropyran-2-yl-indazol-3-yl]pyrazol-1-yl]-1-methyl-propoxy]propan-1-ol as a colorless oil which was used in the next step without further purification.

LCMS method F: [M+H]⁺=529.5, t_{R}=3.34 min

### Preparation of Intermediate 214: 3-[(1S)-3-[4-[5-[tert-butyl(dimethyl)silyl]oxy-1-tetrahydropyran-2-yl-indazol-3-yl]pyrazol-1-yl]-1-methyl-propoxy]propyl methanesulfonate

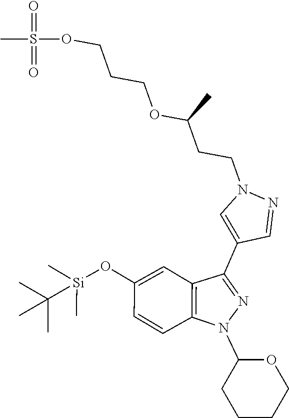 [SMILES: C[C@@H](CCn1cc(-c2nn(C3CCCCO3)c3ccc(O[Si](C)(C)C(C)(C)C)cc23)cn1)OCCCOS(C)(=O)=O]

To a solution of 3-[(1S)-3-[4-[5-[tert-butyl(dimethyl)silyl]oxy-1-tetrahydropyran-2-yl-indazol-3-yl]pyrazol-1-yl]-1-methyl-propoxy]propan-1-ol (343 mg, 0.65 mmol) and triethylamine (181 μL, 1.3 mmol) in dichloromethane (15 mL) at 0° C. was added methanesulfonyl chloride (65 μL, 0.84 mmol) in dichloromethane (5 mL). The reaction mixture was stirred at RT for 1 h. The reaction mixture was diluted with water and extracted with dichloromethane. The combined organic layers were dried over anhydrous sodium sulfate, filtered and evaporated under reduced pressure to afford 3-[(1S)-3-[4-[5-[tert-butyl(dimethyl)silyl]oxy-1-tetrahydropyran-2-yl-indazol-3-yl]pyrazol-1-yl]-1-methyl-propoxy]propyl methanesulfonate as a yellow oil which was used in the next step without further purification.

LCMS method F: [M+H]⁺=607.5, t_{R}=3.49 min

### Preparation of Intermediate 215: (6R)-6-methyl-18-(oxan-2-yl)-9,13-dioxa-4,5,18, 19-tetraazatetracyclo[12.5.2.1^{2,5}.0^{17,20}]docosa-1(19),2(22),3,14(21),15,17(20)-hexaene

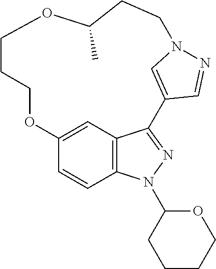 [SMILES: C[C@H]1CCn2cc(cn2)-c2nn(C3CCCCO3)c3ccc(cc23)OCCCO1]

To a suspension of sodium hydride (60% dispersion in mineral oil) (53 mg, 1.32 mmol) in anhydrous DMF (100 mL) at 80° C. was added dropwise 3-[(1S)-3-[4-[5-[tert-butyl(dimethyl) silyl]oxy-1-tetrahydropyran-2-yl-indazol-3-yl]pyrazol-1-yl]-1-methyl-propoxy]propyl methanesulfonate (200 mg, 0.33 mmol) in DMF (100 mL). The reaction mixture was stirred at 80° C. for 30 min. The reaction mixture was concentrated under reduced pressure, diluted with brine and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 60/40 as eluent to afford (6R)-6-methyl-18-(oxan-2-yl)-9,13-dioxa-4,5,18,19-tetraazatetracyclo[12.5.2.1²,0^{17,20}] docosa-1(19),2(22),3,14(21),15,17(20)-hexaene as a yellow oil.

LCMS method F: [M+H]⁺=397.3, t_{R}=2.68 min

### Preparation of Example 37: (8S)-8-methyl-9,13-dioxa-4,5,18,19-tetraazatetracyclo [12.5.2.1^{2,5}.0^{17,20}]docosa-1(19),2(22),3,14(21),15,17(20)-hexaene

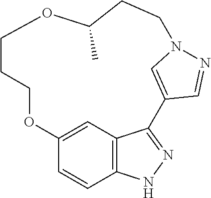 [SMILES: C[C@H]1CCn2cc(cn2)-c2n[nH]c3ccc(cc23)OCCCO1]

To a solution of (6S)-6-methyl-18-(oxan-2-yl)-9,13-dioxa-4,5,18,19-tetraazatetracyclo [12.5.2.1^{2,5}.0^{17,20}]docosa-1(19),2(22),3,14(21),15,17(20)-hexaene (100 g, 0.25 mmol) in methanol (10 mL) and water (1.5 mL) was added p-toluenesulfonic acid monohydrate (240 mg, 1.26 mmol). The reaction mixture was stirred at 65° C. for 2 h. The reaction mixture was concentrated under vacuo and the residue was neutralized by addition of a saturated aqueous NaHCO₃ solution. The residue was diluted with ethyl acetate. The aqueous layer was extracted with ethyl acetate. The combined organic layers were washed with brine, dried over anhydrous sodium sulfate, filtered and evaporated under reduced pressure. The resulting solid was recrystallized from diisopropylether. The resulting product was further purified by chiral HPLC. The resulting residue was triturated with diisopropylether, filtered and dried to afford (8S)-8-methyl-9,13-dioxa-4,5,18,19-tetraazatetracyclo[12.5.2.1^{2,5}.0^{17,20}]docosa-1(19),2(22),3, 14(21),15,17(20)-hexaene as a powder.

LCMS method F: [M+H]⁺=313.3, t_{R}=2.07 min

LCMS method H: [M+H]⁺=313.3, t_{R}=2.06 min

¹H NMR (400 MHz, d6-DMSO) δ 12.68 (1H, s), 8.58 (1H, s), 7.65 (1H, s), 7.41-7.37 (2H, m), 6.96-6.93 (1H, m), 4.44 (1H, dd, J=8.0, 14.2 Hz), 4.32-4.25 (3H, m), 3.70-3.64 (2H, m), 3.51-3.44 (1H, m), 2.17-2.10 (3H, m), 1.95-1.88 (1H, m), 1.22-1.18 (3H, m) ppm.

### Example 38: (6S,12S)-6,12-dimethyl-9,13-dioxa-4,5,18,19-tetraazatetracyclo [12.5.2.1^{2,5}.0^{17,20}]docosa-1(19),2(22),3,14(21),15,17(20)-hexaene

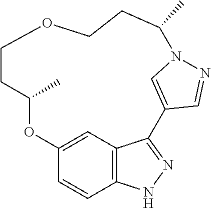 [SMILES: C[C@H]1CCOCC[C@H](C)n2cc(cn2)-c2n[nH]c3ccc(cc23)O1]

Example 38 is prepared according to the synthesis route described in general Scheme D, following the same synthesis procedures as for Example 25.

### Preparation of Intermediate 216: (6S,12S)-6,12-dimethyl-18-(oxan-2-yl)-9,13-dioxa-4,5,18,19-tetraazatetracyclo[12.5.2.^{12,5}.0^{17,20}]docosa-1(19),2(22),3,14(21),15,17(20)-hexaene

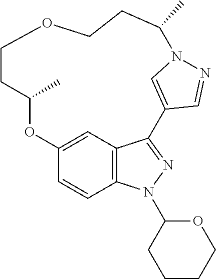 [SMILES: C[C@H]1CCOCC[C@H](C)n2cc(cn2)-c2nn(C3CCCCO3)c3ccc(cc23)O1]

To a heated suspension of cesium carbonate (357 mg, 1.096 mmol) in dry DMF (100 mL) at 80° C. was added dropwise [(1R)-3-[(3S)-3-[4-[5-[tert-butyl(dimethyl)silyl]oxy-1-tetrahydro pyran-2-yl-indazol-3-yl]pyrazol-1-yl]butoxy]-1-methyl-propyl] methanesulfonate (170 mg, 0.274 mmol) in dry DMF (80 mL). The reaction mixture was stirred at 80° C. for 1 h. The solvent was removed under reduced pressure, diluted with brine and extracted with ethyl acetate. The combined organic layers were dried over anhydrous sodium sulfate, filtered and evaporated under reduced pressure. The residue was purified by silica gel column chromatography using dichloromethane/ethyl acetate 100/0 to 60/40 as eluent. The resulting product was further purified by preparative TLC eluting with dichloromethane/methanol 95/5 to afford (6S,12S)-6,12-dimethyl-18-(oxan-2-yl)-9,13-dioxa-4,5,18,19-tetraazatetracyclo[12.5.2.^{12,5}.0^{17,20}] docosa-1(19),2(22),3,14(21),15,17(20)-hexaene as an orange oil.

LCMS method L: [M+H]+=411, t_{R}=2.81 min

### Preparation of Example 38: (6S,12S)-6,12-dimethyl-9,13-dioxa-4,5,18,19-tetraaza tetracyclo[12.5.2.1^{2,5}.0^{17,20}]docosa-1(19),2(22),3,14(21),15,17(20)-hexaene

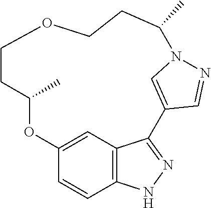 [SMILES: C[C@H]1CCOCC[C@H](C)n2cc(cn2)-c2n[nH]c3ccc(cc23)O1]

To a solution of (6S,12S)-6,12-dimethyl-18-(oxan-2-yl)-9,13-dioxa-4,5,18,19-tetraazatetra cyclo[12.5.2.1^{2,5}.0^{17,20}]docosa-1(19),2(22),3,14(21),15,17(20)-hexaene (13 mg, 0.032 mmol) in methanol (1.9 mL) and water (0.25 mL) was added p-toluenesulfonic acid monohydrate (30.4 mg, 0.160 mmol). The reaction mixture was stirred at 65° C. for 16 h. The reaction mixture was concentrated under reduced pressure and the crude was neutralized by addition of a saturated aqueous NaHCO₃ solution. The aqueous layer was extracted with ethyl acetate. The combined organic layers were washed with brine, dried over anhydrous sodium sulfate, filtered and evaporated under reduced pressure. The residue was purified by preparative TLC eluting with dichloromethane/methanol 95/5 to afford (6S,12S)-6,12-dimethyl-9,13-dioxa-4,5,18,19-tetraazatetracyclo[12.5.2.1^{2,5}.0^{17,20}]docosa-1(19),2(22),3,14(21),15,17(20)-hexaene as a solid.

LCMS method F: [M+H]⁺=327, t_{R}=2.23 min

LCMS method H: [M+H]⁺=327, t_{R}=2.21 min

¹H NMR (400 MHz, MeOD) δ 8.60 (1H, s), 7.79 (1H, s), 7.60 (1H, m), 7.40-7.38 (1H, d), 7.02-6.99 (1H, dd, J=2.3, 8.8 Hz), 4.68-4.55 (2H, m), 3.81-3.76 (1H, m), 3.71-3.55 (3H, m), 2.54-2.38 (2H, m), 2.34-2.25 (1H, m), 1.62 (3H, d, J=6.9 Hz), 1.56-1.48 (1H, m), 1.47-1.45 (3H, d, J=6.1 Hz) ppm.

### Example 39: (13S)-13-methyl-4-(pyrrolidin-1-yl)-7,10-dioxa-5,14,19,20,23-pentaazatetra cyclo[13.5.2.1^{2,6}.0^{18,21}]tricosa-1(20),2(23),3,5,15(22),16,18(21)-heptaene

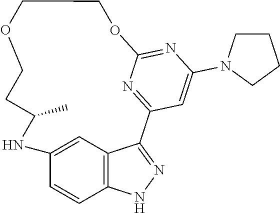 [SMILES: C[C@H]1CCOCCOc2nc(cc(N3CCCC3)n2)-c2n[nH]c3ccc(cc23)N1]

Example 39 is prepared according to the synthesis route described in general Scheme E, following the same synthesis procedures as for Example 30.

The Suzuki reaction was carried out on 5-((tert-butyldimethylsilyl)oxy)-1-(tetrahydro-2H-pyran-2-yl)-3-(4,4,5,5-tetramethyl-1,3,2-dioxaborolan-2-yl)-1H-indazole with 4,6-dichloro-2-(methylthio)pyrimidine to afford 5-((tert-butyldimethylsilyl)oxy)-3-(6-chloro-2-(methylthio) pyrimidin-4-yl)-1-(tetrahydro-2H-pyran-2-yl)-1H-indazole, which was then reacted with pyrrolidine yielding 5-((tert-butyldimethylsilyl)oxy)-3-(2-(methylthio)-6-(pyrrolidin-1-yl) pyrimidin-4-yl)-1-(tetrahydro-2H-pyran-2-yl)-1H-indazole. The next steps were carried out according to the same experimental procedures as for example 30.

### Preparation of Intermediate 217: (13S)-13-methyl-14-(2-nitrobenzenesulfonyl)-19-(oxan-2-yl)-4-(pyrrolidin-1-yl)-7,10-dioxa-5,14,19,20,23-pentaazatetracyclo [13.5.2.1^{2,6}.0^{18,21}]tricosa-1(20),2(23),3,5,15(22),16,18(21)-heptaene

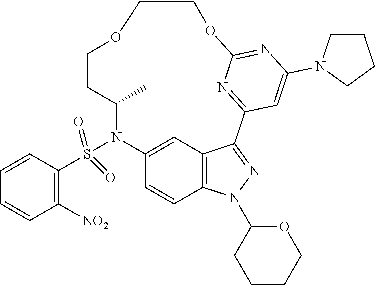 [SMILES: C[C@H]1CCOCCOc2nc(cc(N3CCCC3)n2)-c2nn(C3CCCCO3)c3ccc(cc23)N1S(=O)(=O)c1ccccc1[N+](=O)[O-]]

To a solution of N—((S)-4-(2-hydroxyethoxy)butan-2-yl)-N-(3-(2-(methylsulfonyl)-6-(pyrrolidin-1-yl)pyrimidin-4-yl)-1-(tetrahydro-2H-pyran-2-yl)-1H-indazol-5-yl)-2-nitro benzenesulfonamide (185 mg, 0.249 mmol) in dry THF (13 mL) at 60° C. under nitrogen atmosphere was added dropwise sodium hydride (60% dispersion in mineral oil) (30 mg, 0.747 mmol) in dry THF (12 mL). The reaction mixture was stirred at 60° C. for 1 h. The reaction mixture was cooled at 0° C. diluted with ethyl acetate and quenched with water. The aqueous layer was extracted with ethyl acetate. The combined organic layers were dried over anhydrous magnesium sulfate, filtered and evaporated under reduced pressure. The residue was purified by silica gel column chromatography using heptane/ethyl acetate 100/0 to 50/50 as eluent to afford (13S)-13-methyl-14-(2-nitrobenzenesulfonyl)-19-(oxan-2-yl)-4-(pyrrolidin-1-yl)-7,10-dioxa-5,14,19,20,23-pentaazatetracyclo[13.5.2.1^{2,6}.0^{18,21}]tricosa-1(20),2(23),3,5,15(22),16, 18(21)-heptaene as a white solid.

LCMS method C: [M+H]⁺=664.0, t_{R}=1.29 min

### Preparation of Intermediate 218: (13S)-13-methyl-19-(oxan-2-yl)-4-(pyrrolidin-1-yl)-7,10-dioxa-5,14,19,20,23-pentaazatetracyclo[13.5.2.1^{2,6}.0^{18,21}]tricosa-1(20),2(23), 3,5,15(22),16,18(21)-heptaene

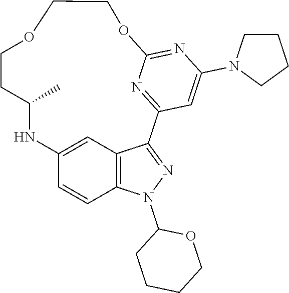 [SMILES: C[C@H]1CCOCCOc2nc(cc(N3CCCC3)n2)-c2nn(C3CCCCO3)c3ccc(cc23)N1]

To a suspension of (13S)-13-methyl-14-(2-nitrobenzenesulfonyl)-19-(oxan-2-yl)-4-(pyrrolidin-1-yl)-7,10-dioxa-5,14,19,20,23-pentaazatetracyclo[13.5.2.1^{2,6}.0^{18,21}]tricosa-1(20), 2(23),3,5,15(22),16,18(21)-heptaene (90 mg, 0.136 mmol) and cesium carbonate (133 mg, 0.408 mmol) in DMF (1.5 mL) at 0° C. was added thiophenol (42 □L, 0.408 mmol). The reaction mixture was stirred at RT for 2 h. The reaction mixture was diluted with ethyl acetate and 1N aqueous sodium hydroxide solution. The aqueous layer was extracted with ethyl acetate. The combined organic layers were dried over anhydrous magnesium sulfate, filtered and evaporated under reduced pressure. The residue was purified by silica gel column chromatography using heptane/ethyl acetate 100/0 to 60/40 as eluent to afford (13S)-13-methyl-19-(oxan-2-yl)-4-(pyrrolidin-1-yl)-7,10-dioxa-5,14,19,20,23-pentaazatetracyclo[13.5.2.1^{2,6}.0^{18,21}]tricosa-1(20),2(23),3,5,15(22),16,18(21)-heptaene as a yellowish solid.

LCMS method B: [M+H]⁺=479.1, t_{R}=1.154 min

### Preparation of Example 39: (13S)-13-methyl-4-(pyrrolidin-1-yl)-7,10-dioxa-5,14,19, 20,23-pentaazatetracyclo[13.5.2.1^{2,6}.0^{18,21}]tricosa-1(20),2(23),3,5,15(22),16,18(21)-heptaene

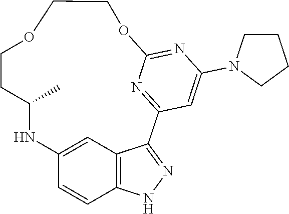 [SMILES: C[C@H]1CCOCCOc2nc(cc(N3CCCC3)n2)-c2n[nH]c3ccc(cc23)N1]

A mixture of (13S)-13-methyl-19-(oxan-2-yl)-4-(pyrrolidin-1-yl)-7,10-dioxa-5,14,19,20,23-pentaazatetracyclo[13.5.2.1^{2,6}.0^{18,21}]tricosa-1(20),2(23),3,5,15(22),16,18(21 (55 mg, 0.115 mmol) and hydrogen chloride (4M solution in dioxane) (1.5 mL) was stirred at RT for 18 h. The reaction mixture was concentrated under reduced pressure and co-evaporated with toluene and methanol. The resulting solid was diluted with dichloromethane, cooled at 0° C. and neutralized with a saturated aqueous NaHCO₃ solution. The aqueous layer was extracted with dichloromethane and chloroform/isopropyl alcohol (3/1) mixture. The combined organic layers were dried over anhydrous magnesium sulfate, filtered and evaporated under reduced pressure. The residue was purified by silica gel column chromatography using heptane/ethyl acetate 100/0 to 60/40 as eluent to afford (13S)-13-methyl-4-(pyrrolidin-1-yl)-7,10-dioxa-5,14,19, 20,23-pentaazatetracyclo[13.5.2.1^{2,6}.0^{18,21}]tricosa-1(20),2(23),3,5,15(22),16,18(21)-heptaene as a cream solid.

LCMS method E: [M+H]⁺=395.1, t_{R}=2.644 min

LCMS method D: [M+H]⁺=395.1, t_{R}=2.469 min

¹H NMR (400 MHz, d6-DMSO) 13.07 (s, 1H), 7.56 (s, 1H), 7.31 (d, J=8.9 Hz, 1H), 6.85 (dd, J=8.9, 2.1 Hz, 1H), 6.80 (s, 1H), 5.41 (brs, 1H), 4.88 (td, J=10.4, 5.3 Hz, 1H), 4.26 (ddd, J=10.6, 7.2, 5.3 Hz, 1H), 4.09 (td, J=10.6, 5.1 Hz, 1H), 3.73 (td, J=11.9, 2.5 Hz, 1H), 3.63 (brt, 1H), 3.56-3.38 (m, 6H), 2.47-2.39 (m, 1H), 1.94 (brs, 4H), 1.24 (d, J=6.5 Hz, 3H), 0.95 (ddt, J=14.6, 9.6, 2.7 Hz, 1H) ppm.

### Example 40: (13S)-4,13-dimethyl-7,10-dioxa-5,14,19,20,23-pentaazatetracyclo[13.5.2.1^{2,6}.0^{18,21}]tricosa-1(20),2(23),3,5,15(22),16,18(21)-heptaene

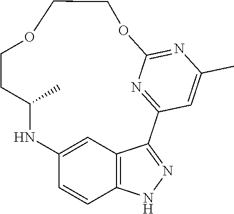 [SMILES: Cc1cc2nc(n1)OCCOCC[C@H](C)Nc1ccc3[nH]nc-2c3c1]

Example 40 is prepared according to the synthesis route described in general Scheme E, following the same synthesis procedures as for Example 30.

The Suzuki reaction was carried out on 5-((tert-butyldimethylsilyl)oxy)-1-(tetrahydro-2H-pyran-2-yl)-3-(4,4,5,5-tetramethyl-1,3,2-dioxaborolan-2-yl)-1H-indazole with 4,6-dichloro-2-(methylthio)pyrimidine to afford 5-((tert-butyldimethylsilyl)oxy)-3-(6-chloro-2-(methylthio) pyrimidin-4-yl)-1-(tetrahydro-2H-pyran-2-yl)-1H-indazole, which was then reacted with trimethylboroxine yielding 5-((tert-butyldimethylsilyl)oxy)-3-(6-methyl-2-(methylthio) pyrimidin-4-yl)-1-(tetrahydro-2H-pyran-2-yl)-1H-indazole.

The next steps were carried out according to the same experimental procedures as for example 30.

### Preparation of Intermediate 219: (13S)-4,13-dimethyl-14-(2-nitrobenzenesulfonyl)-19-(oxan-2-yl)-7,10-dioxa-5,14,19,20,23-pentaazatetracyclo[13.5.2.1^{2,6}.0^{18,21}]tricosa-1(20),2(23),3,5,15(22),16,18(21)-heptaene

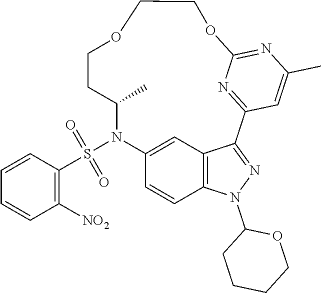 [SMILES: Cc1cc2nc(n1)OCCOCC[C@H](C)N(S(=O)(=O)c1ccccc1[N+](=O)[O-])c1ccc3c(c1)c-2nn3C1CCCCO1]

To a solution of N—((S)-4-(2-hydroxyethoxy)butan-2-yl)-N-(3-(6-methyl-2-(methylsulfonyl) pyrimidin-4-yl)-1-(tetrahydro-2H-pyran-2-yl)-1H-indazol-5-yl)-2-nitrobenzenesulfonamide (320 mg, 0.465 mmol) in dry THF (23.5 mL) at 60° C. under argon was added dropwise a solution of sodium hydride (60% dispersion in mineral oil) (56 mg, 1.395 mmol) in dry THF (23.5 mL). The reaction mixture was stirred at 60° C. for 60 min. The reaction mixture was cooled at 0° C. diluted with ethyl acetate and quenched with water. The aqueous layer was extracted with ethyl acetate. The combined organic layers were dried over anhydrous magnesium sulfate, filtered and evaporated under reduced pressure. The residue was purified by silica gel column chromatography using heptane/ethyl acetate 100/0 to 50/50 as eluent to afford (13S)-4,13-dimethyl-14-(2-nitrobenzenesulfonyl)-19-(oxan-2-yl)-7,10-dioxa-5,14,19, 20,23-pentaazatetracyclo[13.5.2.1^{2,6}.0^{18,21}]tricosa-1(20),2(23),3,5,15(22),16,18(21)-heptaene as a yellowish oil.

LCMS method C: [M+H]⁺=609.3, t_{R}=5.887 and 6.007 min

### Preparation of Intermediate 220: (13S)-4,13-dimethyl-19-(oxan-2-yl)-7,10-dioxa-5,14,19,20,23-pentaazatetracyclo[13.5.2.1^{2,6}.0^{18,21}]tricosa-1(20),2(23),3,5,15(22),16, 18(21)-heptaene

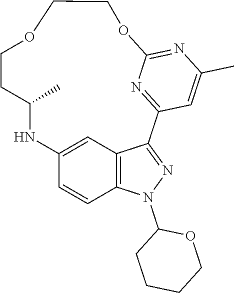 [SMILES: Cc1cc2nc(n1)OCCOCC[C@H](C)Nc1ccc3c(c1)c-2nn3C1CCCCO1]

To a suspension of (13S)-4,13-dimethyl-14-(2-nitrobenzenesulfonyl)-19-(oxan-2-yl)-7,10-dioxa-5,14,19,20,23-pentaazatetracyclo[13.5.2.1^{2,6}.0^{18,21}]tricosa-1(20),2(23),3,5,15(22),16, 18(21)-heptaene (168 mg, 0.276 mmol) and cesium carbonate (270 mg, 0.828 mmol) in DMF (3 mL) at 0° C. was added thiophenol (0.084 ml, 0.828 mmol). The reaction mixture was stirred at RT for 2 h. The reaction mixture was diluted with ethyl acetate and 1N aqueous sodium hydroxide solution and the mixture was extracted with ethyl acetate. The combined organic layers were dried over anhydrous magnesium sulfate, filtered and evaporated under reduced pressure. The residue was purified by silica gel column chromatography using heptane/ethyl acetate 100/0 to 60/40 as eluent to afford (13S)-4,13-dimethyl-19-(oxan-2-yl)-7,10-dioxa-5,14,19,20,23-pentaazatetracyclo[13.5.2.1^{2,6}.0^{18,21}]tricosa-1(20),2(23),3,5,15(22),16,18(21)-heptaene as a yellow solid.

LCMS method B: [M+H]⁺=424.0, t_{R}=1.076 min

### Preparation of Example 40: (13S)-4,13-dimethyl-7,10-dioxa-5,14,19,20,23-penta azatetracyclo[13.5.2.1^{2,6}.0^{18,21}]tricosa-1(20),2(23),3,5,15(22),16,18(21)-heptaene

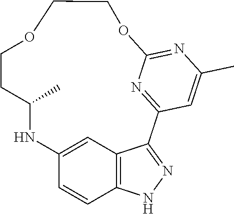 [SMILES: Cc1cc2nc(n1)OCCOCC[C@H](C)Nc1ccc3[nH]nc-2c3c1]

A mixture of (13S)-4,13-dimethyl-19-(oxan-2-yl)-7,10-dioxa-5,14,19,20,23-pentaazatetra cyclo[13.5.2.1^{2,6}.0^{18,21}]tricosa-1(20),2(23),3,5,15(22),16,18(21)-heptaene (82 mg, 0.194 mmol) and hydrogen chloride (4M in dioxane) (4 mL) was stirred at RT for 72 h. The reaction mixture was concentrated under reduced pressure and co-evaporated with toluene and methanol. The resulting solid was diluted with dichloromethane, cooled to 0° C. and neutralized with a saturated aqueous NaHCO₃ solution. The aqueous layer was extracted with dichloromethane. The combined organic layers were dried over anhydrous magnesium sulfate, filtered and evaporated under reduced pressure. The residue was purified by silica gel column chromatography using heptane/ethyl acetate 100/0 to 40/60 as eluent. to afford (13S)-4,13-dimethyl-7,10-dioxa-5,14,19,20,23-pentaazatetracyclo[13.5.2.1^{2,6}.0^{18,21}]tricosa-1(20),2(23),3, 5,15(22),16,18(21)-heptaene as a solid.

LCMS method E: [M+H]⁺=340.1, t_{R}=2.451 min

LCMS method D: [M+H]⁺=340.1, t_{R}=2.138 min

¹H NMR (400 MHz, d6-DMSO) 13.33 (s, 1H), 7.61 (s, 1H), 7.55 (d, J=2.2 Hz, 1H), 7.35 (d, J=8.9 Hz, 1H), 6.88 (dd, J=9.0, 2.1 Hz, 1H), 5.51 (d, J=6.5 Hz, 1H), 4.99-4.84 (m, 1H), 4.32-4.13 (m, 2H), 3.73 (td, J=11.8, 2.5 Hz, 1H), 3.65 (dtd, J=9.4, 6.1, 2.6 Hz, 1H), 3.53 (ddd, J=10.3, 5.1, 2.6 Hz, 2H), 2.47-2.42 (m, 1H), 2.41 (s, 3H), 1.25 (d, J=6.5 Hz, 3H), 0.98 (ddt, J=14.6, 9.7, 2.9 Hz, 1H) ppm.

### Example 41: (13R)-13-methyl-7,10,14-trioxa-4,19,20,23-tetraazatetracyclo[13.5.2.1^{2,6}.0^{18,21}]tricosa-1(20),2(23),3,5,15(22),16,18(21)-heptaene

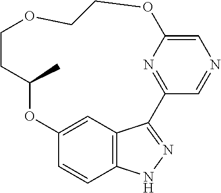 [SMILES: C[C@@H]1CCOCCOc2cncc(n2)-c2n[nH]c3ccc(cc23)O1]

Example 41 is prepared according to the synthesis route described in general Scheme D.

### Preparation of Intermediate 221: 2-[2-[(3S)-3-benzyloxybutoxy]ethoxy]tetrahydro pyran

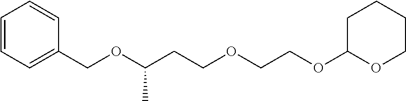 [SMILES: C[C@@H](CCOCCOC1CCCCO1)OCc1ccccc1]

[(3S)-3-benzyloxybutyl]-4-methylbenzenesulfonate was prepared according to the same procedures as [(3R)-3-benzyloxybutyl] 4-methylbenzenesulfonate (intermediate 129) starting from (3S)-butane-1,3-diol.

To a solution of 2-tetrahydropyran-2-yloxyethanol (730 mg, 5 mmol) in dry DMF (20 mL) at 0° C. was added portionwise sodium hydride (60% dispersion in mineral oil) (300 mg, 7.5 mmol). The reaction mixture was stirred at 0° C. for 10 min. A solution of [(3S)-3-benzyloxybutyl] 4-methylbenzenesulfonate (2 g, 6 mmol) in dry DMF (5 mL) was added dropwise and the reaction mixture was stirred at 70° C. overnight. The reaction mixture was quenched with water and ethyl acetate was added. The layers were separated. The aqueous layer was extracted with ethyl acetate. The combined organic layers were dried over anhydrous sodium sulfate, filtered and evaporated under reduced pressure. The residue was purified by silica gel column chromatography using cyclohexane/ethyl acetate: 90/10 to 80/20 as eluent to afford 2-[2-[(3S)-3-benzyloxybutoxy]ethoxy]tetrahydropyran as a colorless oil.

LCMS method F: [M+Na]⁺=331.0, t_{R}=2.83 min

### Preparation of Intermediate 222: 2-[(3S)-3-benzyloxybutoxy]ethanol

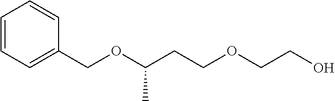 [SMILES: C[C@@H](CCOCCO)OCc1ccccc1]

To a solution of 2-[2-[(3S)-3-benzyloxybutoxy]ethoxy]tetrahydropyran (1.2 g, 3.89 mmol) in methanol (30 mL) and water (6 mL) was added p-toluenesulfonic acid monohydrate (2.22 g, 11.97 mmol). The reaction mixture was stirred at 65° C. for 3 h. The reaction mixture was cooled to RT and the solvent was removed under reduced pressure. The residue was diluted with a saturated aqueous solution of NaHCO₃ and ethyl acetate. The layers were separated and the aqueous layer was extracted with ethyl acetate. The combined organic layers were washed with brine, dried over anhydrous sodium sulfate, filtered and evaporated under reduced pressure to afford 2-[(3S)-3-benzyloxybutoxy]ethanol as a pale yellow oil which was used in the next step without further purification.

LCMS method F: [M+H]⁺=225.2, t_{R}=2.07 min

### Preparation of Intermediate 223: 2-[2-[(3S)-3-benzyloxybutoxy]ethoxy]-6-chloro-pyrazine

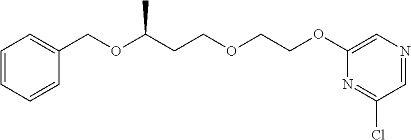 [SMILES: C[C@@H](CCOCCOc1cncc(Cl)n1)OCc1ccccc1]

To a solution of 2-[(3 S)-3-benzyloxybutoxy]ethanol (112 mg, 0.5 mmol) in THF (4 mL) at 0° C. was added sodium hydride (60% dispersion in mineral oil) (24 mg, 0.6 mmol). The reaction mixture was stirred at 0° C. for 30 min. 2,6-Dichloropyrazine (74 mg, 0.5 mmol) was added and the solution was stirred at 0° C. for 2 h.

The reaction mixture was quenched by addition of a saturated NH₄Cl solution and the aqueous layer was extracted with ethyl acetate. The combined organic layers were washed with brine, dried over anhydrous sodium sulfate, filtered and evaporated under reduced pressure to afford 2-[2-[(3S)-3-benzyloxybutoxy]ethoxy]-6-chloro-pyrazine as a pale yellow oil which was used in the next step without further purification.

LCMS method F: [M+H]⁺=337.1, t_{R}=3.01 min

### Preparation of Intermediate 224: [3-[6-[2-[(3S)-3-benzyloxybutoxy]ethoxy]pyrazin-2-yl]-1-tetrahydropyran-2-yl-indazol-5-yl]oxy-tert-butyl-dimethyl-silane

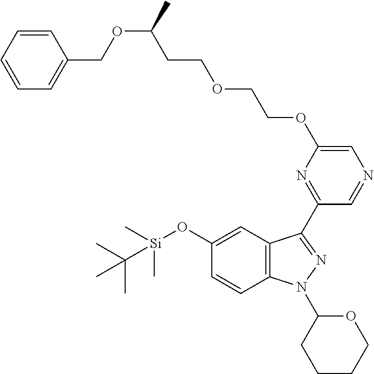 [SMILES: C[C@@H](CCOCCOc1cncc(-c2nn(C3CCCCO3)c3ccc(O[Si](C)(C)C(C)(C)C)cc23)n1)OCc1ccccc1]

To a degassed solution of tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (238 mg, 0.52 mmol), 2-[2-[(3S)-3-benzyloxybutoxy]ethoxy]-6-chloro-pyrazine (146 mg, 0.43 mmol) potassium phosphate tribasic (276 mg, 1.3 mmol) in dioxane (6 mL) and water (0.6 mL) was added tetrakis(triphenylphosphine)palladium(0) (25 mg, 0.021 mmol). The reaction mixture was stirred at 100° C. for 3 h. The reaction mixture was cooled to RT and diluted with water. The aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford [3-[6-[2-[(3S)-3-benzyloxybutoxy]ethoxy]pyrazin-2-yl]-1-tetrahydropyran-2-yl-indazol-5-yl]oxy-tert-butyl-dimethyl-silane as a colorless oil.

LCMS method I: [M+H]⁺=633.4, t_{R}=3.81 min

### Preparation of Intermediate 225: (2S)-4-[2-[6-[5-[tert-butyl(dimethyl)silyl]oxy-1-tetrahydropyran-2-yl-indazol-3-yl]pyrazin-2-yl]oxyethoxy]butan-2-ol

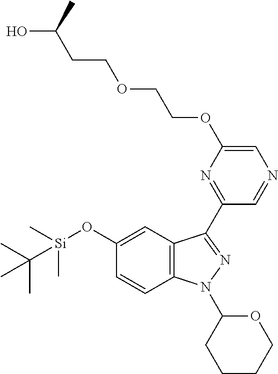 [SMILES: C[C@H](O)CCOCCOc1cncc(-c2nn(C3CCCCO3)c3ccc(O[Si](C)(C)C(C)(C)C)cc23)n1]

To a solution of [3-[6-[2-[(3S)-3-benzyloxybutoxy]ethoxy]pyrazin-2-yl]-1-tetrahydropyran-2-yl-indazol-5-yl]oxy-tert-butyl-dimethyl-silane (1 g, 1.58 mmol) in ethyl acetate (25 mL) under a nitrogen atmosphere was added 10% palladium on carbon (100 mg). The solution was stirred at RT under hydrogen atmosphere for 7 days. The reaction mixture was filtered and the filtrate was evaporated under reduced pressure to afford (2S)-4-[2-[6-[5-[tert-butyl(dimethyl)silyl] oxy-1-tetrahydropyran-2-yl-indazol-3-yl]pyrazin-2-yl]oxyethoxy]butan-2-ol as a pale yellow oil which was used in the next step without further purification.

LCMS method F: [M+H]⁺=543.3, t_{R}=3.56 min

### Preparation of Intermediate 226: [(1S)-3-[2-[6-[5-[tert-butyl(dimethyl)silyl]oxy-1-tetrahydropyran-2-yl-indazol-3-yl]pyrazin-2-yl]oxyethoxy]-1-methyl-propyl]methane sulfonate

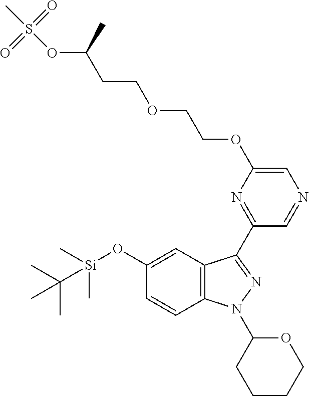 [SMILES: C[C@@H](CCOCCOc1cncc(-c2nn(C3CCCCO3)c3ccc(O[Si](C)(C)C(C)(C)C)cc23)n1)OS(C)(=O)=O]

To a solution of (2S)-4-[2-[6-[5-[tert-butyl(dimethyl)silyl]oxy-1-tetrahydropyran-2-yl-indazol-3-yl]pyrazin-2-yl]oxyethoxy]butan-2-ol (800 mg, 1.47 mmol) and triethylamine (410 μL, 2.94 mmol) in dichloromethane (20 mL) at 0° C. was added methanesulfonyl chloride (148 μL, 1.91 mmol) in dichloromethane (5 mL). The reaction mixture was stirred at RT for 3 h. Additional triethylamine (410 μL, 2.94 mmol) and methanesulfonyl chloride (148 μL, 1.91 mmol) in dichloromethane (5 mL) were added and the reaction mixture was stirred at RT overnight. The reaction mixture was diluted with dichloromethane and the organic layer was washed with brine, dried over anhydrous sodium sulfate, filtered and evaporated under reduced pressure to afford [(1S)-3-[2-[6-[5-[tert-butyl(dimethyl)silyl]oxy-1-tetrahydropyran-2-yl-indazol-3-yl] pyrazin-2-yl]oxyethoxy]-1-methyl-propyl]methanesulfonate as a yellow oil which was used in the next step without further purification.

LCMS method F: [M+H]⁺=621.3, t_{R}=3.65 min

### Preparation of Intermediate 227: (13R)-13-methyl-19-(oxan-2-yl)-7,10,14-trioxa-4,19,20,23-tetraazatetracyclo[13.5.2.1^{2,6}.0^{18,21}]tricosa-1(20),2(23),3,5,15(22),16, 18(21)-heptaene

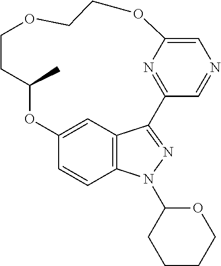 [SMILES: C[C@@H]1CCOCCOc2cncc(n2)-c2nn(C3CCCCO3)c3ccc(cc23)O1]

To a solution of sodium hydride (60% dispersion in mineral oil) (86.8 mg, 2.17 mmol) in DMF (100 mL) was added dropwise [(1S)-3-[2-[6-[5-[tert-butyl(dimethyl)silyl]oxy-1-tetrahydro pyran-2-yl-indazol-3-yl]pyrazin-2-yl]oxyethoxy]-1-methyl-propyl]methanesulfonate (450 mg, 0.72 mmol) in DMF (50 mL). The reaction mixture was stirred at RT overnight. The reaction mixture was quenched with water and concentrated under reduced pressure. The solid residue was suspended in water and stirred for 2 h, filtered and dried. The residue was purified by silica gel column chromatography using cyclohexane/ethyl acetate 90/10 to 70/30 as eluent to afford (13R)-13-methyl-19-(oxan-2-yl)-7,10,14-trioxa-4,19,20,23-tetraazatetracyclo[13.5.2.1^{2,6}.0^{18,21}]tricosa-1(20),2(23),3,5,15(22),16,18(21)-heptaene as a pink solid.

LCMS method F: [M+H]⁺=411.2, t_{R}=3.23 min

### Preparation of Example 41: (13R)-13-methyl-7,10,14-trioxa-4,19,20,23-tetraazatetra cyclo[13.5.2.1^{2,6}.0^{18,21}]tricosa-1(20),2(23),3, 5,15(22),16,18(21)-heptaene

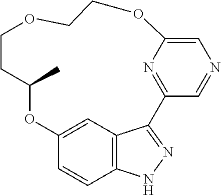 [SMILES: C[C@@H]1CCOCCOc2cncc(n2)-c2n[nH]c3ccc(cc23)O1]

To a suspension of (13R)-13-methyl-19-(oxan-2-yl)-7,10,14-trioxa-4,19,20,23-tetraazatetra cyclo[13.5.2.1^{2,6}.0^{18,21}]tricosa-1(20),2(23),3,5,15(22),16,18(21)-heptaene (390 mg, 0.95 mmol) in methanol (70 mL) and water (10 mL) was added p-toluenesulfonic acid monohydrate (903 mg, 4.75 mmol). The reaction mixture was stirred at 65° C. for 24 h. The solvent was evaporated under reduced pressure and a saturated solution of NaHCO₃ solution was added. The aqueous layer was extracted with ethyl acetate. The combined organic layers were washed with brine, dried over anhydrous sodium sulfate, filtered and evaporated under reduced pressure. The resulting solid was triturated with diisopropylether, filtered and dried under reduced pressure to afford (13R)-13-methyl-7,10,14-trioxa-4,19,20,23-tetraazatetracyclo[13.5.2.1^{2,6}.0^{18,21}] tricosa-1(20),2(23),3,5,15(22),16,18(21)-heptaene as a solid.

LCMS method F: [M+H]⁺=327.2, t_{R}=2.54 min

LCMS method: [M+H]⁺=327.2, t_{R}=2.47 min

¹H NMR (400 MHz, CDCl₃) δ 10.24 (1H, s), 9.09 (1H, s), 8.29 (1H, d, J=2.3 Hz), 8.18-8.17 (1H, s), 7.44 (1H, d, J=9.0 Hz), 7.13 (1H, dd, J=2.4, 9.0 Hz), 5.17-5.10 (1H, m), 4.82-4.76 (1H, m), 4.43-4.36 (1H, m), 4.29-4.23 (1H, m), 3.79-3.74 (3H, m), 2.51-2.42 (1H, m), 1.57 (1H, m), 1.47 (3H, d, J=6.3 Hz) ppm.

### Example 42: (12R)-12-methyl-9,13-dioxa-4,5,18,19-tetraazatetracyclo[12.5.2.1^{2,5}.0^{17,20}] docosa-1(19),2(22),3, 14(21),15,17(20)-hexaene

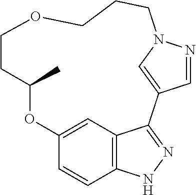 [SMILES: C[C@@H]1CCOCCCn2cc(cn2)-c2n[nH]c3ccc(cc23)O1]

Example 42 is prepared according to the synthesis route described in general Scheme F.

### Preparation of Intermediate 228: trimethyl-[2-[[4-(4,4,5,5-tetramethyl-1,3,2-dioxa borolan-2-yl)pyrazol-1-yl]methoxy]ethyl]silane

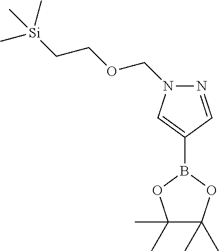

To a solution of 4-(4,4,5,5-tetramethyl-1,3,2-dioxaborolan-2-yl)-1H-pyrazole (1.94 g, 10 mmol) in NMP (15 mL) at RT was added potassium carbonate (2.76 g, 20 mmol) and 2-(trimethylsilyl)ethoxymethyl chloride (1.86 mL, 10.5 mmol). The reaction mixture was stirred at RT for 48 h. The reaction mixture was diluted with ethyl acetate and filtered. The filtrate was washed with water then brine. The organic layer was dried over anhydrous sodium sulfate, filtered and evaporated under reduced pressure. The residue was purified by silica gel column chromatography using cyclohexane/ethyl acetate 100/0 to 90/10 as eluent to afford trimethyl-[2-[[4-(4,4,5,5-tetramethyl-1,3,2-dioxaborolan-2-yl)pyrazol-1-yl]methoxy]ethyl]silane as a yellow oil.

LCMS method F: [M+H]⁺=325.2, t_{R}=3.07 min

### Preparation of Intermediate 229: 1-tetrahydropyran-2-yl-3-[1-(2-trimethylsilylethoxy methyl)pyrazol-4-yl]indazol-5-ol

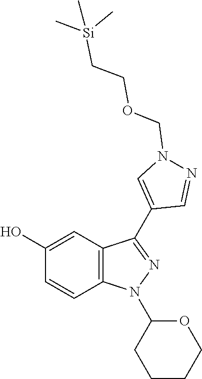 [SMILES: C[Si](C)(C)CCOCn1cc(-c2nn(C3CCCCO3)c3ccc(O)cc23)cn1]

To a solution of 3-iodo-1-tetrahydropyran-2-yl-indazol-5-ol (intermediate 16) (1.617 g, 4.70 mmol) in dioxane (14.1 mL) and water (4.7 mL) at RT was added trimethyl-[2-[[4-(4,4,5,5-tetramethyl-1,3,2-dioxaborolan-2-yl)pyrazol-1-yl]methoxy]ethyl]silane (2.134 g, 6.58 mmol), potassium phosphate tribasic (2.993 g, 14.1 mmol), XPhos (224 mg, 0.47 mmol) and tetrakis(triphenylphosphine)palladium(0) (272 mg, 0.24 mmol). The reaction mixture was stirred under microwave irradiations at 100° C. for 1 h. The reaction mixture was diluted with brine and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 60/40 as eluent to afford 1-tetrahydropyran-2-yl-3-[1-(2-trimethylsilylethoxymethyl)pyrazol-4-yl]indazol-5-ol as a white solid.

LCMS method F: [M+H]⁺=415.2, t_{R}=2.89 min

### Preparation of Intermediate 230: benzyl 2-[[4-[5-[(1R)-3-(3-benzyloxypropoxy)-1-methyl-propoxy]-1-tetrahydropyran-2-yl-indazol-3-yl]pyrazol-1-yl]methoxy]ethyl-trimethyl-silane

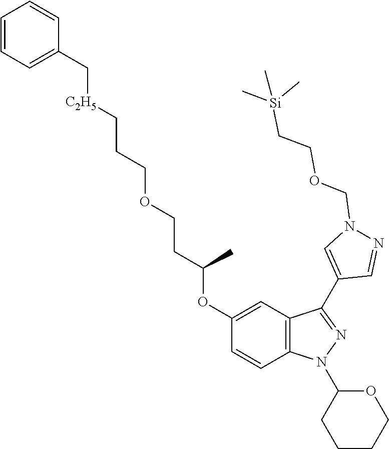

To a solution of 1-tetrahydropyran-2-yl-3-[1-(2-trimethylsilylethoxymethyl)pyrazol-4-yl] indazol-5-ol (880 mg, 2.12 mmol) in DMF (20 mL) was added cesium carbonate (1.037 g, 3.18 mmol) and [(1S)-3-(3-benzyloxypropoxy)-1-methyl-propyl]methanesulfonate (intermediate 79) (739 mg, 2.33 mmol). The reaction mixture was stirred at 70° C. for 24 h. The reaction mixture was filtered and concentrated under reduced pressure. The residue was dissolved in ethyl acetate and water was added. The aqueous layer was extracted with ethyl acetate. The combined organic layers were washed with brine, dried over anhydrous sodium sulfate, filtered and evaporated under reduced pressure. The residue was purified by silica gel column chromatography using dichloromethane/ethyl acetate 100/0 to 50/50 as eluent to afford benzyl 2-[[4-[5-[(1R)-3-(3-benzyloxypropoxy)-1-methyl-propoxy]-1-tetrahydropyran-2-yl-indazol-3-yl]pyrazol-1-yl]methoxy]ethyl-trimethyl-silane as a yellow oil.

LCMS method F: [M+H]⁺=635.6, t_{R}=3.75 min

### Preparation of Intermediate 231: 3-[(3R)-3-[1-tetrahydropyran-2-yl-3-[1-(2-trimethylsilylethoxymethyl)pyrazol-4-yl]indazol-5-yl]oxybutoxy]propan-1-ol

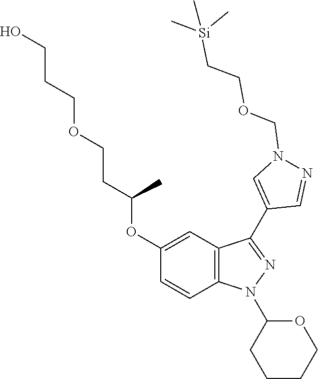 [SMILES: C[C@H](CCOCCCO)Oc1ccc2c(c1)c(-c1cnn(COCC[Si](C)(C)C)c1)nn2C1CCCCO1]

To a solution of 2-[[4-[5-[(1R)-3-(3-benzyloxypropoxy)-1-methyl-propoxy]-1-tetrahydro pyran-2-yl-indazol-3-yl]pyrazol-1-yl]methoxy]ethyl-trimethyl-silane (1.050 g, 1.65 mmol) in ethyl acetate (10 mL) at RT was added palladium 10% on carbon (105 mg). The reaction mixture was stirred under hydrogen atmosphere at RT for 48 h. The reaction mixture was filtered and the filtrate was concentrated under reduced pressure. The residue was purified by silica gel column chromatography using dichloromethane/ethyl acetate 100/0 to 20/80 as eluent to afford 3-[(3R)-3-[1-tetrahydropyran-2-yl-3-[1-(2-trimethylsilylethoxymethyl)pyrazol-4-yl] indazol-5-yl]oxybutoxy]propan-1-ol as a colorless oil.

LCMS method F: [M+H]⁺=545.4, t_{R}=3.13 min

### Preparation of Intermediate 232: 3-[(3R)-3-[3-(1H-pyrazol-4-yl)-1-tetrahydropyran-2-yl-indazol-5-yl]oxybutoxy]propan-1-ol

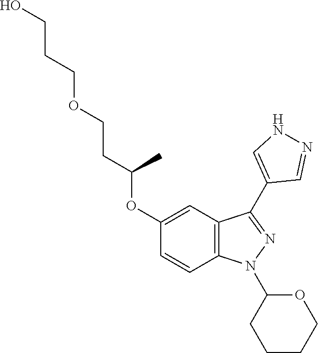 [SMILES: C[C@H](CCOCCCO)Oc1ccc2c(c1)c(-c1cn[nH]c1)nn2C1CCCCO1]

To a solution of 3-[(3R)-3-[1-tetrahydropyran-2-yl-3-[1-(2-trimethylsilylethoxymethyl) pyrazol-4-yl]indazol-5-yl]oxybutoxy]propan-1-ol (530 mg, 0.97 mmol) in THF (10 mL) at RT was added TBAF (1M solution in THF) (1.95 mL, 1.95 mmol). The reaction mixture was stirred at 60° C. for 30 h. The reaction mixture was quenched with a saturated aqueous ammonium chloride solution and extracted with ethyl acetate. The combined organic layers were dried over anhydrous sodium sulfate, filtered and evaporated under reduced pressure. The residue was purified by silica gel column chromatography using dichloromethane/ethyl acetate 100/0 to 60/40 as eluent to afford 3-[(3R)-3-[3-(1H-pyrazol-4-yl)-1-tetrahydropyran-2-yl-indazol-5-yl] oxybutoxy]propan-1-ol as a colorless oil.

LCMS method F: [M+H]⁺=415.4, t_{R}=2.17 min

### Preparation of Intermediate 233: 3-[(3R)-3-[3-(1H-pyrazol-4-yl)-1-tetrahydropyran-2-yl-indazol-5-yl]oxybutoxy]propyl 4-methylbenzenesulfonate

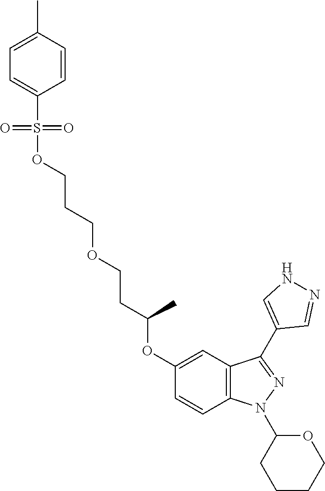 [SMILES: Cc1ccc(S(=O)(=O)OCCCOCC[C@@H](C)Oc2ccc3c(c2)c(-c2cn[nH]c2)nn3C2CCCCO2)cc1]

To a solution of 3-[(3R)-3-[3-(1H-pyrazol-4-yl)-1-tetrahydropyran-2-yl-indazol-5-yl]oxy butoxy]propan-1-ol (150 mg, 0.36 mmol) in pyridine (2 mL) at −10° C. was added dropwise p-toluenesulfonyl chloride (76 mg, 0.40 mmol) in pyridine (1 mL). The reaction mixture was allowed to warm up to RT and stirred at for 1 h. The reaction mixture was diluted with a saturated aqueous NaHCO₃ solution and extracted with ethyl acetate. The combined organic layers were washed with water and brine, dried over anhydrous sodium sulfate, filtered and evaporated under reduced pressure to afford 3-[(3R)-3-[3-(1H-pyrazol-4-yl)-1-tetrahydropyran-2-yl-indazol-5-yl]oxybutoxy]propyl 4-methylbenzenesulfonate as a colorless oil which was used in the next step without further purification.

LCMS method F: [M+H]⁺=569.3, t_{R}=2.98 min

### Preparation of Intermediate 234: (12R)-12-methyl-18-(oxan-2-yl)-9,13-dioxa-4,5,18, 19-tetraazatetracyclo[12.5.2.1^{2,5}.0^{17,20}]docosa-1(19),2(22),3,14(21),15,17(20)-hexaene

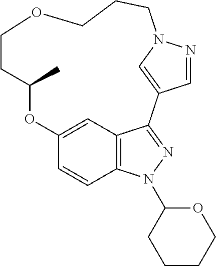 [SMILES: C[C@@H]1CCOCCCn2cc(cn2)-c2nn(C3CCCCO3)c3ccc(cc23)O1]

To a suspension of cesium carbonate (472 mg, 1.45 mmol) in DMF (144 mL) at 90° C. was added dropwise 3-[(3R)-3-[3-(1H-pyrazol-4-yl)-1-tetrahydropyran-2-yl-indazol-5-yl]oxy butoxy]propyl 4-methylbenzenesulfonate (206 mg, 0.36 mmol) in DMF (144 mL). The reaction mixture was stirred at 90° C. for 1 h. The solvent was evaporated under reduced pressure and the residue was diluted with brine and extracted with ethyl acetate. The combined organic layers were dried over anhydrous sodium sulfate, filtered and evaporated under reduced pressure. The residue was purified by silica gel column chromatography using dichloromethane/methanol 100/0 to 98/2 as eluent to afford (12R)-12-methyl-18-(oxan-2-yl)-9,13-dioxa-4,5,18,19-tetra azatetracyclo[12.5.2.1^{2,5}.0^{17,20}]docosa-1(19),2(22),3,14(21),15,17(20)-hexaene as a colorless oil.

LCMS method F: [M+H]⁺=397.2, t_{R}=3.90 min

### Preparation of Example 42: (12R)-12-methyl-9,13-dioxa-4,5,18,19-tetraazatetracyclo [12.5.2.1^{2,5}.0^{17,20}]docosa-1(19),2(22),3,14(21),15,17(20)-hexaene

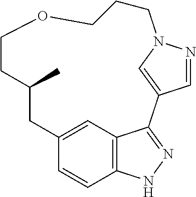 [SMILES: C[C@@H]1CCOCCCn2cc(cn2)-c2n[nH]c3ccc(cc23)C1]

To a solution of (12R)-12-methyl-18-(oxan-2-yl)-9,13-dioxa-4,5,18,19-tetraazatetracyclo [12.5.2.1^{2,5}.0^{17,20}]docosa-1(19),2(22),3,14(21),15,17(20)-hexaene (40 mg, 0.10 mmol) in methanol (3.5 mL) and water (0.5 mL) was added p-toluenesulfonic acid monohydrate (96 mg, 0.50 mmol). The reaction mixture was stirred at 65° C. for 2 h. The solvent was evaporated under reduced pressure and the residue was neutralized by slow addition of a saturated aqueous NaHCO₃ solution. The aqueous layer was extracted with ethyl acetate. The combined organic layers were washed with brine, dried over anhydrous sodium sulfate, filtered and evaporated under reduced pressure. The resulting solid was recrystallized from acetonitrile to afford (12R)-12-methyl-9,13-dioxa-4,5,18,19-tetraazatetracyclo[12.5.2.1^{2,5}.0^{17,20}]docosa-1(19),2(22),3, 14(21),15,17(20)-hexaene as a solid.

LCMS method F: [M+H]⁺=313.2, t_{R}=2.15 min

LCMS method G: [M+H]⁺=313.2, t_{R}=2.10 min H NMR (400 MHz, d6-DMSO) 12.67 (1H, s), 8.63 (1H, s), 7.67 (1H, d, J=0.8 Hz), 7.46 (1H, d, J=2.5 Hz), 7.40-7.37 (1H, m), 6.93 (1H, dd, J=2.5, 8.9 Hz), 4.54-4.42 (2H, m), 4.35-4.27 (1H, m), 3.75-3.51 (4H, m), 2.50-2.40 (1H, m), 2.22-2.07 (2H, m), 1.55-1.45 (1H, m), 1.41 (3H, d, J=5.9 Hz) ppm.

### Example 43: (13S)-13-methyl-10,14-dioxa-19,20-diazatetracyclo[13.5.2.1^{2,6}.0^{18,21}]tricosa-1(20),2(23),3,5,15(22),16,18(21)-heptaene

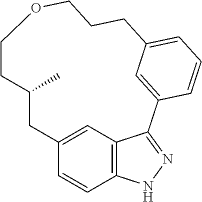 [SMILES: C[C@H]1CCOCCCc2cccc(c2)-c2n[nH]c3ccc(cc23)C1]

Example 43 is prepared according to the synthesis route described in general Scheme D.

### Preparation of Intermediate 235: (3R)-3-tetrahydropyran-2-yloxybutanoate

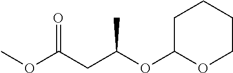

To a solution of methyl (3R)-3-hydroxybutanoate (5 g, 42.3 mmol) in diethyl ether (45 mL) at 0° C. was added p-toluenesulfonic acid monohydrate (80 mg, 4.2 mmol) and 3,4-dihydro-2H-pyran (5.8 mL, 63.5 mmol). The reaction mixture was stirred at 0° C. for 1 h then RT overnight. Saturated aqueous NaHCO₃ solution was added. The aqueous layer was extracted with diethyl ether. The combined organic layers were dried over anhydrous magnesium sulfate, filtered and evaporated under reduced pressure to afford methyl (3R)-3-tetrahydropyran-2-yloxybutanoate as a colorless oil which was used in the next step without further purification.

¹H NMR (400 MHz, DMSO) 4.72-4.63 (1H, m), 4.13-4.01 (1H, m), 3.81-3.67 (1H, m) 3.60-3.58 (3H, m), 3.46-3.30 (1H, m), 2.56-2.52 (1H, m), 2.49-2.41 (1H, m), 1.73-1.52 (2H, m), 1.51-1.32 (4H, m), 1.20-1.09 (3H, m) ppm.

### Preparation of Intermediate 236: (3R)-3-tetrahydropyran-2-yloxybutan-1-ol

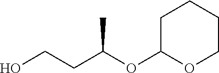

To a solution of methyl (3R)-3-tetrahydropyran-2-yloxybutanoate (3.75 g, 18.5 mmol) in dry THF (40 mL) at 0° C. under nitrogen atmosphere was added dropwise LiAlH₄ (1.0 M solution in THF) (18.5 mL, 18.5 mmol). The reaction mixture was stirred at 0° C. under atmosphere of nitrogen for 1 h. The reaction mixture was quenched at 0° C. by addition of water (760 μL) in 5 mL of THF, 1M aqueous sodium hydroxide solution (3.32 mL) and water (2.28 mL). After stirring at RT for 30 min, the precipitate was filtered through a pad of Celite, washed with ethyl acetate and the solvent was removed under reduced pressure. The residue was purified on silica gel column chromatography using cyclohexane/ethyl acetate 100/0 to 50/50 as eluent to afford (3R)-3-tetrahydropyran-2-yloxybutan-1-ol as a colorless oil.

¹H NMR (400 MHz, DMSO) 4.68-4.59 (1H, m), 4.38-4.29 (1H, m), 3.87-3.73 (2H, m), 3.52-3.38 (3H, m), 1.79-1.34 (8H, m), 1.16-1.03 (3H, m) ppm.

### Preparation of Intermediate 237: [(3R)-3-tetrahydropyran-2-yloxybutyl]-4-methyl benzenesulfonate

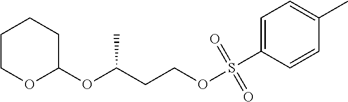 [SMILES: Cc1ccc(S(=O)(=O)OCC[C@@H](C)OC2CCCCO2)cc1]

To a solution of methyl (3R)-3-tetrahydropyran-2-yloxybutan-1-ol (2 g, 11.48 mmol) in pyridine (8 mL) at 0° C. was added p-toluenesulfonyl chloride (2.408 g, 12.63 mmol) The reaction mixture was stirred at RT for 5 h. The solvent was revaporated under reduced pressure and the residue was diluted with ethyl acetate and a saturated aqueous NaHCO₃ solution. The aqueous layer was extracted with ethyl acetate. The combined organic layers were washed with brine, dried over anhydrous magnesium sulfate, filtered and evaporated under reduce pressure to afford [(3R)-3-tetrahydropyran-2-yloxybutyl] 4-methylbenzenesulfonate as a pale yellow liquid which was used in the next step without further purification.

LCMS method F: [M+H]⁺=351.1, t_{R}=2.84 min

### Preparation of Intermediate 238: 3-(3-bromophenyl)propan-1-ol

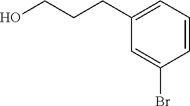 [SMILES: OCCCc1cccc(Br)c1]

To a solution of 3-(3-bromophenyl)propionic acid (1.5 g, 6.55 mmol) in anhydrous THF (60 mL) was added BH₃ (1.0 M solution in THF) (13.1 mL, 13.1 mmol). The reaction mixture was refluxed for 2 h then quenched with water (50 mL) and 1N aqueous HCl solution (25 mL). The aqueous layer extracted with ethyl acetate. The organic layer was washed with brine and dried over anhydrous magnesium sulfate, filtered and evaporated under reduced pressure to afford 3-(3-bromophenyl)propan-1-ol as a colorless oil which was used in the next step without further purification.

LCMS method F: [M+H]⁺=217, t_{R}=2.29 min

### Preparation of Intermediate 239: 2-[(1R)-3-[3-(3-bromophenyl)propoxy]-1-methyl-propoxy]tetrahydropyran

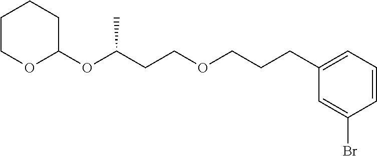 [SMILES: C[C@H](CCOCCCc1cccc(Br)c1)OC1CCCCO1]

To a solution of 3-(3-bromophenyl)propan-1-ol (970 mg, 4.50 mmol) in dry DMF (10 mL) at 0° C. was added portionwise sodium hydride (60% dispersion in mineral oil) (200 mg, 6.50 mmol). The reaction mixture was stirred at 0° C. for 10 min then a solution of [(3R)-3-tetrahydropyran-2-yloxybutyl] 4-methylbenzenesulfonate (1.772 g, 5.4 mmol) in dry DMF (10.0 mL) was added dropwise. The reaction mixture was allowed to warm up to RT and stirred for 16 h The solvent was removed under reduced pressure. The residue was diluted with a saturated aqueous NaHCO₃ solution and ethyl acetate. The aqueous layer was extracted with ethyl acetate. The combined organic layers were dried over anhydrous sodium sulfate, filtered and evaporated under reduced pressure. The residue was purified by silica gel column chromatography using cyclohexane/ethyl acetate 90/10 to 80/20 as eluent to afford 2-[(1R)-3-[3-(3-bromophenyl)propoxy]-1-methyl-propoxy]tetrahydropyran as a colorless oil.

LCMS method F: [M+Na]⁺=393.1-395.1, t_{R}=3.39 min

### Preparation of Intermediate 240: (2R)-4-[3-(3-bromophenyl)propoxy]butan-2-ol

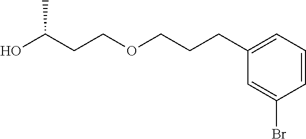 [SMILES: C[C@@H](O)CCOCCCc1cccc(Br)c1]

To a solution of 2-[(1R)-3-[3-(3-bromophenyl)propoxy]-1-methyl-propoxy]tetrahydropyran (675 mg, 1.82 mmol) in methanol (28 mL) and water (7 mL) was added p-toluenesulfonic acid (519 mg, 2.73 mmol). The reaction mixture was stirred at RT for 5 h. The solvent was evaporated under reduced pressure. The residue was diluted with a saturated aqueous NaHCO₃ solution and ethyl acetate. The aqueous layer was extracted with ethyl acetate. The combined organic layers were washed with brine, dried over anhydrous magnesium sulfate, filtered and evaporated under reduced pressure to afford (2R)-4-[3-(3-bromophenyl)propoxy]butan-2-ol as a pale yellow oil which was used in the next step without further purification.

LCMS method F: [M+H]⁺=287.1-289.1, t_{R}=2.65 min

### Preparation of Intermediate 241: (2R)-4-[3-[3-[5-[tert-butyl(dimethyl)silyl]oxy-1-tetrahydropyran-2-yl-indazol-3-yl]phenyl]propoxy]butan-2-ol

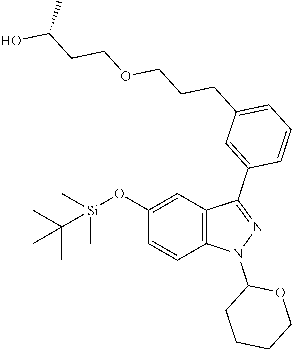 [SMILES: C[C@@H](O)CCOCCCc1cccc(-c2nn(C3CCCCO3)c3ccc(O[Si](C)(C)C(C)(C)C)cc23)c1]

To a degassed solution of [5-[tert-butyl(dimethyl)silyl]oxy-1-tetrahydropyran-2-yl-indazol-3-yl]boronic acid (482 mg, 1.282 mmol), (2R)-4-[3-(3-bromophenyl)propoxy]butan-2-ol (368 mg, 1.282 mmol), potassium phosphate tribasic (815 mg, 3.846 mmol) and XPhos (61 mg, 0.128 mmol) in dioxane (11.7 mL) and water (2.3 mL) was added tetrakis(triphenylphosphine) palladium(0) (15 mg, 0.064 mmol). The reaction mixture was stirred at 90° C. for 4 h. Water (25 ml) and ethyl acetate (25 ml) were added. The aqueous layer was extracted with ethyl acetate. The combined organic layers were washed with brine, dried over anhydrous magnesium sulfate, filtered and evaporated under reduced pressure to afford (2R)-4-[3-[3-[5-[tert-butyl(dimethyl) silyl]oxy-1-tetrahydropyran-2-yl-indazol-3-yl]phenyl] propoxy]butan-2-ol as a pale yellow oil which was used in the next step without further purification.

LCMS method F: [M+H]⁺=539.3, t_{R}=3.79 min

### Preparation of Intermediate 242: [(1R)-3-[3-[3-[5-[tert-butyl(dimethyl)silyl]oxy-1-tetrahydropyran-2-yl-indazol-3-yl]phenyl]propoxy]-1-methyl-propyl]methane sulfonate

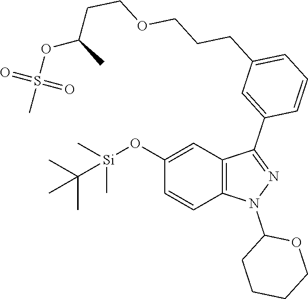 [SMILES: C[C@H](CCOCCCc1cccc(-c2nn(C3CCCCO3)c3ccc(O[Si](C)(C)C(C)(C)C)cc23)c1)OS(C)(=O)=O]

To a solution of (2R)-4-[3-[3-[5-[tert-butyl(dimethyl)silyl]oxy-1-tetrahydropyran-2-yl-indazol-3-yl]phenyl]propoxy]butan-2-ol (430 mg, 0.798 mmol) and triethylamine (170 μL, 1.197 mmol) in dichloromethane (20 mL) at 0° C. was added methanesulfonyl chloride (110 μL, 0.958 mmol). The reaction mixture was stirred at 0° C. for 5 min then warmed up to RT and stirred for 16 h. Saturated aqueous ammonium chloride solution (2 mL) was added. The aqueous layer was extracted with dichloromethane and the combined organic layers were washed with a saturated aqueous NaHCO₃ solution and brine, dried over anhydrous sodium sulfate, filtered and the solvent was removed under reduced pressure to afford [(1R)-3-[3-[3-[5-[tert-butyl(dimethyl)silyl]oxy-1-tetrahydropyran-2-yl-indazol-3-yl]phenyl]propoxy]-1-methyl-propyl]methanesulfonate as a pale yellow oil which was used in the next step without further purification.

LCMS method F: [M+H]⁺=617.3, t_{R}=3.82 min

### Preparation of Intermediate 243: (13S)-13-methyl-19-(oxan-2-yl)-10,14-dioxa-19,20-diazatetracyclo[13.5.2.1^{2,6}.0^{18,21}]tricosa-1(20), 2(23),3,5,15(22),16,18(21)-heptaene

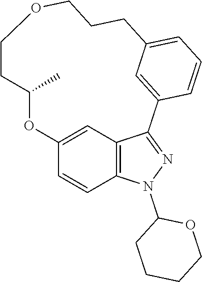 [SMILES: C[C@H]1CCOCCCc2cccc(c2)-c2nn(C3CCCCO3)c3ccc(cc23)O1]

To a suspension of cesium carbonate (1.022 g, 3.144 mmol) in dry DMF (40 mL) was added dropwise [(1R)-3-[3-[3-[5-[tert-butyl(dimethyl)silyl]oxy-1-tetrahydropyran-2-yl-indazol-3-yl] phenyl]propoxy]-1-methyl-propyl] methanesulfonate (485 mg, 0.786 mmol) in dry DMF (10 mL) and the reaction mixture was stirred at 70° C. for 2 h. The reaction mixture was filtered and the solvent was removed under reduced pressure. The residue was purified by silica gel column chromatography using cyclohexane/ethyl acetate 100/0 to 80/20 as eluent to afford (13S)-13-methyl-19-(oxan-2-yl)-10,14-dioxa-19,20-diazatetracyclo[13.5.2.1^{2,6}.0^{18,21}]tricosa-1(20), 2(23),3,5,15(22),16,18(21)-heptaene as a colorless oil.

LCMS method F: [M+H]⁺=407.3, t_{R}=3.56 min

### Preparation of Example 43: (13S)-13-methyl-10,14-dioxa-19,20-diazatetracyclo [13.5.2.1^{2,6}.0^{18,21}]tricosa-1(20),2(23),3,5,15(22),16,18(21)-heptaene

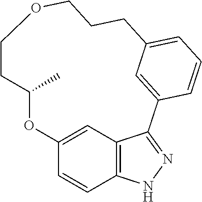 [SMILES: C[C@H]1CCOCCCc2cccc(c2)-c2n[nH]c3ccc(cc23)O1]

To a solution of (13S)-13-methyl-19-(oxan-2-yl)-10,14-dioxa-19,20-diazatetracyclo[13.5.2.1^{2,6}.0^{18,21}]tricosa-1(20),2(23),3,5,15(22),16,18(21)-heptaene (160 mg, 0.394 mmol) in methanol (4.5 mL) and water (0.5 mL) was added p-toluenesulfonic acid monohydrate (374 mg, 1.968 mmol). The reaction mixture was stirred at 65° C. for 2 h. The solvent was evaporated under reduced pressure. A saturated aqueous NaHCO₃ solution and ethyl acetate were added. The aqueous layer was extracted with ethyl acetate, and the organic layer was washed with brine. The organic layer was dried over anhydrous magnesium sulfate, filtered and evaporated under reduce pressure. The residue was purified by silica gel column chromatography using cyclohexane/ethyl acetate 100/0 to 70/30 as eluent. The resulting solid was triturated with dichloromethane, filtered and dried to afford (13S)-13-methyl-10,14-dioxa-19,20-diazatetra cyclo[13.5.2.1^{2,6}.0^{18,21}]tricosa-1(20),2(23),3,5,15(22),16,18(21)-heptaene as a powder.

LCMS method F: [M+H]⁺=323.3, t_{R}=2.90 min

LCMS method G: [M+H]⁺=323.3, t_{R}=2.87 min

¹H NMR (400 MHz, d6-DMSO) 13.01 (1H, s), 8.08 (1H, t, J=1.3 Hz), 7.75-7.72 (2H, m), 7.48-7.45 (1H, m), 7.38 (1H, t, J=7.7 Hz), 7.17-7.15 (1H, m), 6.98 (1H, dd, J=2.1, 8.9 Hz), 4.57 (1H, s), 4.12-4.08 (3H, m), 3.78-3.67 (1H, m), 3.58-3.53 (3H, m), 2.96-2.85 (1H, m), 2.13-2.03 (1H, m), 1.95-1.84 (1H, m), 1.40 (3H, d, J=6.1 Hz) ppm.

### Example 44: (8R,13S)-4-(3-methoxyazetidin-1-yl)-8,13-dimethyl-7,10,14-trioxa-5,19,20, 23-tetraazatetracyclo[13.5.2.1^{2,6}.0^{18,21}]tricosa-1(20),2(23),3,5,15(22),16, 18(21)-heptaene

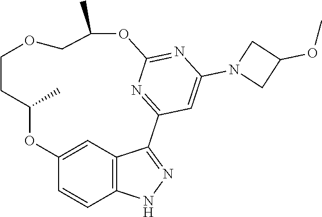 [SMILES: COC1CN(c2cc3nc(n2)O[C@H](C)COCC[C@H](C)Oc2ccc4[nH]nc-3c4c2)C1]

Example 44 is prepared according to the synthesis route described in general Scheme C.

### Preparation of Intermediate 244: 4-chloro-6-(3-methoxyazetidin-1-yl)-2-methyl sulfanyl-pyrimidine

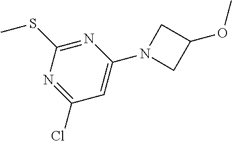

To a solution of 4,6-dichloro-2-methylsulfanyl-pyrimidine (500 mg, 2.58 mmol) and DIPEA (900 μL, 5.16 mmol) in THF (25 mL) was added 3-methoxyazetidine hydrochloride (319 mg, 2.58 mmol). The reaction mixture was stirred at 60° C. for 72 h. The reaction mixture was cooled to RT and diluted with ethyl acetate and water. The organic layer was washed with brine, dried over anhydrous magnesium sulfate, filtered and evaporated under reduced pressure to afford 4-chloro-6-(3-methoxyazetidin-1-yl)-2-methylsulfanyl-pyrimidine as a colorless oil which solidified upon standing.

LCMS method F: [M+H]⁺=246.2, t_{R}=2.38 min

### Preparation of Intermediate 245: tert-butyl-[3-[6-(3-methoxyazetidin-1-yl)-2-methyl sulfanyl-pyrimidin-4-yl]-1-tetrahydropyran-2-yl-indazol-5-yl]oxy-dimethyl-silane

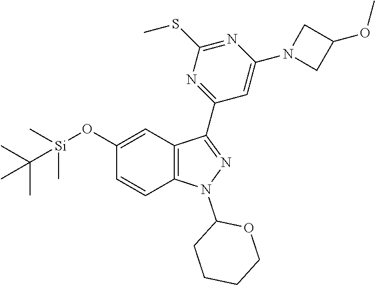 [SMILES: COC1CN(c2cc(-c3nn(C4CCCCO4)c4ccc(O[Si](C)(C)C(C)(C)C)cc34)nc(SC)n2)C1]

In a Schlenk tube, tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (710 mg, 1.55 mmol), 4-chloro-6-(3-methoxyazetidin-1-yl)-2-methylsulfanyl-pyrimidine (320 mg, 1.29 mmol), XPhos (62 mg, 0.13 mmol) and potassium phosphate tribasic (110 mg, 3.87 mmol) were suspended in a mixture of dioxane (12.5 mL) and water (0.5 mL). The reaction mixture was degassed with N₂ for 15 min. Tetrakis(triphenylphosphine)palladium(0) (75 mg, 0.07 mmol) was added and the vial was sealed and heated at 90° C. for 2 h. The mixture was cooled to RT and directly purified by silica gel column chromatography using cyclohexane/ethyl acetate 100/0 to 80/20 as eluent to afford tert-butyl-[3-[6-(3-methoxyazetidin-1-yl)-2-methylsulfanyl-pyrimidin-4-yl]-1-tetrahydropyran-2-yl-indazol-5-yl]oxy-dimethyl-silane as a white solid.

LCMS method I: [M+H]⁺=542.4, t_{R}=3.55 min

### Preparation of Intermediate 246: tert-butyl-[3-[6-(3-methoxyazetidin-1-yl)-2-methyl sulfonyl-pyrimidin-4-yl]-1-tetrahydropyran-2-yl-indazol-5-yl]oxy-dimethyl-silane

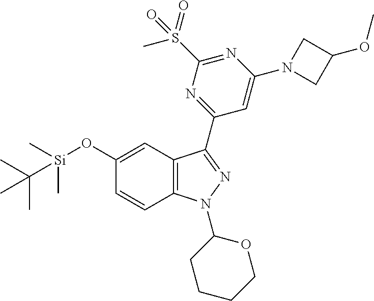 [SMILES: COC1CN(c2cc(-c3nn(C4CCCCO4)c4ccc(O[Si](C)(C)C(C)(C)C)cc34)nc(S(C)(=O)=O)n2)C1]

To a solution of tert-butyl-[3-[6-(3-methoxyazetidin-1-yl)-2-methylsulfanyl-pyrimidin-4-yl]-1-tetrahydropyran-2-yl-indazol-5-yl]oxy-dimethyl-silane (970 mg, 1.79 mmol) in ethanol (40 mL) at 0° C. was added hexaammonium heptamolybdate tetrahydrate (66 mg, 0.054 mmol) and hydrogen peroxide (3.6 mL, 35.8 mmol). The reaction mixture was stirred at RT for 24 h. Additional hydrogen peroxide (1.62 mL, 16.11 mmol) was added and the reaction mixture was stirred at RT for 48 h. Additional hydrogen peroxide (0.54 mL, 5.37 mmol) was added and the reaction mixture was stirred at RT for 24 h. The reaction mixture was filtered and the solid was rinsed with ethanol. The solvent was removed under reduce pressure to afford tert-butyl-[3-[6-(3-methoxyazetidin-1-yl)-2-methylsulfonyl-pyrimidin-4-yl]-1-tetrahydropyran-2-yl-indazol-5-yl]oxy-dimethyl-silane as a white powder which was used in the next step without further purification.

LCMS method F: [M+H]⁺=574.3, t_{R}=3.49 min

### Preparation of Intermediate 247: 3-[2-[(1R)-2-[(3R)-3-benzyloxybutoxy]-1-methyl-ethoxy]-6-(3-methoxyazetidin-1-yl)pyrimidin-4-yl]-1-tetrahydropyran-2-yl-indazol-5-ol

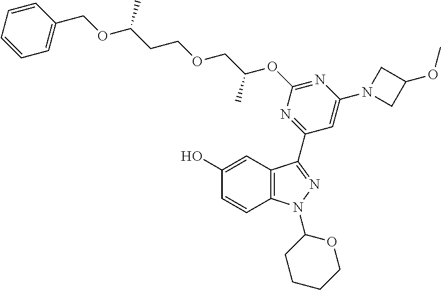 [SMILES: COC1CN(c2cc(-c3nn(C4CCCCO4)c4ccc(O)cc34)nc(O[C@H](C)COCC[C@@H](C)OCc3ccccc3)n2)C1]

To a suspension of sodium hydride (60% dispersion in mineral oil) (25 mg, 0.62 mmol) in DMF (3 mL) at 0° C. was added (2R)-1-[(3R)-3-benzyloxybutoxy]propan-2-ol (intermediate 133) (133 mg, 0.56 mmol). The reaction mixture was stirred at 0° C. for 30 min and a solution of tert-butyl-[3-[6-(3-methoxyazetidin-1-yl)-2-methylsulfonyl-pyrimidin-4-yl]-1-tetrahydropyran-2-yl-indazol-5-yl]oxy-dimethyl-silane (320 mg, 0.56 mmol) in DMF (2.6 mL) was added dropwise. The reaction mixture was stirred at 0° C. for 5 min then warms up to RT and stirred for 16 h. The reaction mixture was quenched with a saturated aqueous ammonium chloride solution and poured in ethyl acetate. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent to afford 3-[2-[(1R)-2-[(3R)-3-benzyloxybutoxy]-1-methyl-ethoxy]-6-(3-methoxyazetidin-1-yl)pyrimidin-4-yl]-1-tetrahydropyran-2-yl-indazol-5-ol as a white foam.

LCMS method F: [M+H]⁺=618.4, t_{R}=2.65 min

### Preparation of Intermediate 248: 3-[2-[(1R)-2-[(3R)-3-hydroxybutoxy]-1-methyl-ethoxy]-6-(3-methoxyazetidin-1-yl)pyrimidin-4-yl]-1-tetrahydropyran-2-yl-indazol-5-ol

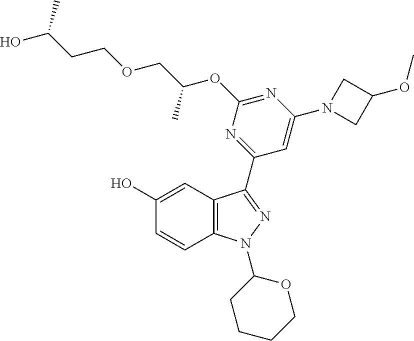 [SMILES: COC1CN(c2cc(-c3nn(C4CCCCO4)c4ccc(O)cc34)nc(O[C@H](C)COCC[C@@H](C)O)n2)C1]

To a solution of 3-[2-[(1R)-2-[(3R)-3-benzyloxybutoxy]-1-methyl-ethoxy]-6-(3-methoxy azetidin-1-yl)pyrimidin-4-yl]-1-tetrahydropyran-2-yl-indazol-5-ol (180 mg, 0.29 mmol) in ethyl acetate (29 mL) was added palladium on charcoal 10% (32 mg, 0.03 mmol). The reaction mixture was stirred under hydrogen atmosphere at 20° C. for 96 h. The reaction mixture was filtered and the solvent was removed under reduced pressure. The residue was purified by silica gel column chromatography using cyclohexane/ethyl acetate 100/0 to 50/50 as eluent to afford 3-[2-[(1R)-2-[(3R)-3-hydroxybutoxy]-1-methyl-ethoxy]-6-(3-methoxyazetidin-1-yl) pyrimidin-4-yl]-1-tetrahydropyran-2-yl-indazol-5-ol as a yellow oil.

LCMS method F: [M+H]⁺=528.4, t_{R}=1.97 min

### Preparation of Intermediate 249: (8R,13S)-4-(3-methoxyazetidin-1-yl)-8,13-dimethyl-19-(oxan-2-yl)-7,10,14-trioxa-5,19,20,23-tetraazatetracyclo[13.5.2.1^{2,6}.0^{18,21}]tricosa-1(20),2(23),3,5,15(22), 16,18(21)-heptaene

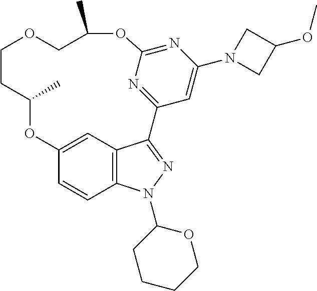 [SMILES: COC1CN(c2cc3nc(n2)O[C@H](C)COCC[C@H](C)Oc2ccc4c(c2)c-3nn4C2CCCCO2)C1]

To a solution of diisopropyl azodicarboxylate (125 μL, 0.63 mmol) in THF (5 mL) at 60° C. was added triphenylphosphine (165 mg, 0.63 mmol) in toluene (10 mL) and a solution of 3-[2-[(1R)-2-[(3R)-3-hydroxybutoxy]-1-methyl-ethoxy]-6-(3-methoxyazetidin-1-yl)pyrimidin-4-yl]-1-tetrahydropyran-2-yl-indazol-5-ol (140 mg, 0.21 mmol) in THF (5 mL). The reaction mixture was stirred at 60° C. for 1 h. The solvent was removed under reduced pressure. The residue was partitioned between ethyl acetate and water. The layers were separated and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford (8R,13S)-4-(3-methoxyazetidin-1-yl)-8,13-dimethyl-19-(oxan-2-yl)-7,10,14-trioxa-5,19,20,23-tetraazatetracyclo[13.5.2.1^{2,6}.0^{18,21}]tricosa-1(20),2(23),3,5,15(22), 16,18(21)-heptaene as a colorless oil.

LCMS method F: [M+H]⁺=510.4, t_{R}=3.32 min

### Preparation of Example 44: (8R,13S)-4-(3-methoxyazetidin-1-yl)-8,13-dimethyl-7,10,14-trioxa-5,19,20,23-tetraazatetracyclo[13.5.2.1^{2,6}.0^{18,21}]tricosa-1(20),2(23),3, 5, 15(22),16,18(21)-heptaene

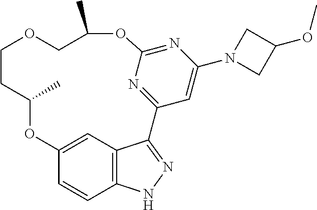 [SMILES: COC1CN(c2cc3nc(n2)O[C@H](C)COCC[C@H](C)Oc2ccc4[nH]nc-3c4c2)C1]

To a solution of (8R,13S)-4-(3-methoxyazetidin-1-yl)-8,13-dimethyl-19-(oxan-2-yl)-7,10,14-trioxa-5,19,20,23-tetraazatetracyclo[13.5.2.1^{2,6}.0^{18,21}]tricosa-1(20),2(23),3,5,15(22),16, 18(21)-heptaene (120 mg, 0.16 mmol) in methanol (14 mL) and water (2 mL) was added p-toluenesulfonic acid monohydrate (152 mg, 0.80 mmol). The reaction mixture was stirred at 65° C. for 24 h. The solvent was evaporated under reduced pressure and the residue was neutralized by addition of a saturated aqueous NaHCO₃ solution. The residue was diluted with ethyl acetate. The aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent. The resulting solid was further purified by preparative HPLC to afford (8R,13S)-4-(3-methoxyazetidin-1-yl)-8,13-dimethyl-7,10,14-trioxa-5,19,20,23-tetraazatetracyclo[13.5.2.1^{2,6}.0^{18,21}]tricosa-1(20),2(23),3,5,15(22),16, 18(21)-heptaene as a powder.

LCMS method F: [M+H]⁺=426.5, t_{R}=3.79 min

LCMS method G: [M+H]⁺=426.5, t_{R}=3.83 min

¹H NMR (400 MHz, d6-DMSO) 13.43 (1H, s), 8.03 (1H, d, J=2.5 Hz), 7.49 (1H, d, J=9.1 Hz), 6.99 (1H, dd, J=2.3, 8.9 Hz), 6.71 (1H, s), 5.13-5.08 (1H, m), 4.65-4.60 (1H, m), 4.39-4.33 (1H, m), 4.28-4.21 (3H, m), 3.91-3.86 (2H, m), 3.79-3.73 (1H, m), 3.60-3.54 (1H, m), 3.27 (3H, s), 2.38-2.31 (1H, m), 1.47-1.43 (2H, m), 1.42 (3H, d, J=6.2 Hz), 1.37 (3H, d, J=6.1 Hz) ppm.

### Example 45: (13R)-13-methyl-7,10,14-trioxa-5,19,20,23-tetraazatetracyclo[13.5.2.1^{2,6}.0^{18,21}]tricosa-1(20),2(23),3,5, 15(22),16,18(21)-heptaene

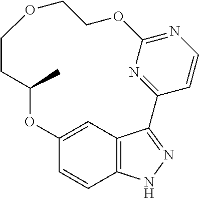 [SMILES: C[C@@H]1CCOCCOc2nccc(n2)-c2n[nH]c3ccc(cc23)O1]

Example 45 is prepared according to the synthesis route described in general Scheme C, following the same synthesis procedures as for Example 16. A Mitsunobu reaction is used for the macrocyclization step.

### Preparation of Intermediate 250: (13R)-13-methyl-19-(oxan-2-yl)-7,10,14-trioxa-5,19,20,23-tetraazatetracyclo[13.5.2.1^{2,6}.0^{18,21}]tricosa-1(20),2(23),3,5,15(22),16, 18(21)-heptaene

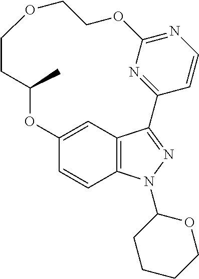 [SMILES: C[C@@H]1CCOCCOc2nccc(n2)-c2nn(C3CCCCO3)c3ccc(cc23)O1]

To a solution of 3-[2-[2-[(3S)-3-hydroxybutoxy]ethoxy]pyrimidin-4-yl]-1-tetrahydropyran-2-yl-indazol-5-ol (130 mg, 0.303 mmol) in dry Me-THF (0.40 mL) and toluene (1.70 ml) was added triphenylphosphine (159 mg, 0.606 mmol). The solution was stirred at 0° C. for 30 min and DIAD (0.119 mL, 0.606 mmol) was added dropwise. The reaction mixture was stirred at RT for 3 h. The solvent was removed under reduced pressure. The residue was purified by silica gel column chromatography using cyclohexane/ethyl acetate 100/0 to 60/40 as eluent to afford (13R)-13-methyl-19-(oxan-2-yl)-7,10,14-trioxa-5,19,20,23-tetraazatetracyclo[13.5.2.1^{2,6}.0^{18,21}]tricosa-1(20),2(23),3,5,15(22),16,18(21)-heptaene.

LCMS method F: [M+H]⁺=411.4, t_{R}=3.03 min

### Preparation of Example 45: (13R)-13-methyl-7,10,14-trioxa-5,19,20,23-tetraazatetra cyclo[13.5.2.1^{2,6}.0^{18,21}]tricosa-1(20),2(23),3, 5,15(22),16,18(21)-heptaene

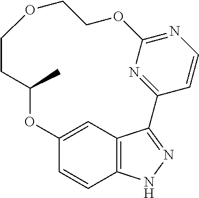 [SMILES: C[C@@H]1CCOCCOc2nccc(n2)-c2n[nH]c3ccc(cc23)O1]

To a solution of (13R)-13-methyl-19-(oxan-2-yl)-7,10,14-trioxa-5,19,20,23-tetraazatetracyclo [13.5.2.1^{2,6}.0^{18,21}]tricosa-1(20),2(23),3,5,15(22),16,18(21)-heptaene (100 mg, 0.244 mmol) in methanol (4.5 mL) and water (0.5 mL) was added p-toluenesulfonic acid monohydrate (232 mg, 1.2 mmol). The reaction mixture was stirred at 65° C. for 16 h. The solvent was evaporated under reduced pressure and the residue was dissolved in ethyl acetate. A saturated aqueous NaHCO₃ solution was added. The aqueous layer was extracted with ethyl acetate. The combined organic layers were washed with brine, dried over anhydrous magnesium sulfate, filtered and evaporated under reduced pressure. The residue was purified by silica gel column chromatography using cyclohexane/ethyl acetate 100/0 to 60/40 as eluent to afford (13R)-13-methyl-7,10,14-trioxa-5,19,20,23-tetraazatetracyclo[13.5.2.1^{2,6}.0^{18,21}]tricosa-1(20),2(23),3, 5, 15(22),16,18(21)-heptaene as a powder.

LCMS method F: [M+H]⁺=327.3, t_{R}=2.29 min

LCMS method G: [M+H]⁺=327.2, t_{R}=2.27 min

¹H NMR (400 MHz, d6-DMSO) δ 13.70 (1H, s), 8.60 (1H, d, J=5.1 Hz), 8.16-8.15 (1H, m), 7.76 (1H, d, J=5.1 Hz), 7.57-7.54 (1H, m), 7.03 (1H, dd, J=2.5, 8.9 Hz), 5.01-4.90 (1H, m), 4.75-4.63 (1H, m), 4.40-4.27 (1H, m), 4.23-4.13 (1H, m), 3.71-3.65 (3H, m), 2.42-2.33 (1H, m), 1.40 (3H, d, J=6.1 Hz), 1.24 (1H, s) ppm.

### Example 46: (8S,13S)-8,13-dimethyl-7,10,14-trioxa-5,19,20,23-tetraazatetracyclo [13.5.2.1^{2,6}.0^{18,21}]tricosa-1(20),2(23),3,5,15(22),16,18(21)-heptaene

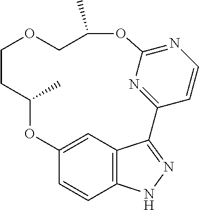 [SMILES: C[C@H]1CCOC[C@H](C)Oc2nccc(n2)-c2n[nH]c3ccc(cc23)O1]

Example 46 is prepared according to the synthesis route described in general Scheme C.

### Preparation of Intermediate 251: [[(3R)-3-allyloxybutoxy]-diphenyl-methyl]benzene

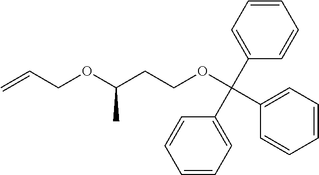 [SMILES: C=CCO[C@H](C)CCOC(c1ccccc1)(c1ccccc1)c1ccccc1]

A solution of (2R)-4-trityloxybutan-2-ol (intermediate 126) (3 g, 9.02 mmol) in dry DMF (80 mL) was degassed N₂ for 15 min. The reaction mixture was cooled to 0° C. and sodium hydride (60% dispersion in mineral oil) (433 mg, 10.82 mmol) was added portionwise. The reaction mixture was stirred at 0° C. for 10 min and a solution of allyl bromide (1.16 mL, 13.54 mmol) in dry DMF (20 mL) was added dropwise at 0° C. The reaction mixture was warmed up to RT and stirred for 16 h. The reaction mixture was cooled to 0° C. and quenched by water. The aqueous layer was extracted with ethyl acetate. The combined organic layers were washed with brine, dried over anhydrous magnesium sulfate, filtered and evaporated under reduced pressure. The residue was purified by silica gel column chromatography using cyclohexane/ethyl acetate 100/0 to 90/10 as eluent afford [[(3R)-3-allyloxybutoxy]-diphenyl-methyl]benzene as a colorless oil.

LCMS method F: [M+Na]⁺=395.2, t_{R}=3.62 min

### Preparation of Intermediate 252: (3R)-3-allyloxybutan-1-ol

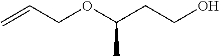

To [[(3R)-3-allyloxybutoxy]-diphenyl-methyl]benzene (3 g, 8.06 mmol) was added HCl (4M in 1,4-dioxane) (20 mL, 80.6 mmol). The reaction mixture was stirred at 20° C. for 1 h. The solvent was evaporated under reduced pressure and the residue was purified by silica gel column chromatography using cyclohexane/ethyl acetate 100/0 to 80/20 as eluent to afford (3R)-3-allyloxybutan-1-ol as a colorless oil.

¹H NMR (400 MHz, CDCl₃), 5.99-5.88 (1H, m), 5.32-5.26 (1H, m), 5.19 (1H, ddt, J=1.3, 4.5, 5.2 Hz), 4.15-4.10 (1H, m), 3.97-3.91 (1H, m), 3.86-3.72 (3H, m), 2.72 (1H, s), 1.80-1.75 (2H, m), 1.23-1.21 (3H, m) ppm.

### Preparation of Intermediate 253: [(3R)-3-allyloxybutyl] 4-methylbenzenesulfonate

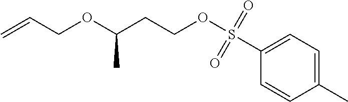 [SMILES: C=CCO[C@H](C)CCOS(=O)(=O)c1ccc(C)cc1]

To a solution of (3R)-3-allyloxybutan-1-ol (900 mg, 6.92 mmol) in pyridine (3.5 mL) at 0° C. was added dropwise p-toluenesulfonyl chloride (1.455 g, 7.61 mmol). The reaction mixture was stirred at RT for 16 h. The solvent was evaporated under reduced pressure and the residue was dissolved in ethyl acetate. The organic layer was washed with a saturated aqueous NaHCO₃ solution and brine, dried over anhydrous magnesium sulfate, filtered and evaporated under reduced pressure to afford [(3R)-3-allyloxybutyl] 4-methylbenzenesulfonate as a colorless oil which solidified upon standing. The product was used in the next step without further purification.

LCMS method F: [M+Na]⁺=307.2, t_{R}=2.75 min

### Preparation of Intermediate 254: 2-[(1S)-2-[(3R)-3-allyloxybutoxy]-1-methyl-ethoxy] tetrahydropyran

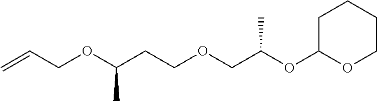 [SMILES: C=CCO[C@H](C)CCOC[C@H](C)OC1CCCCO1]

(2S)-2-tetrahydropyran-2-yloxypropan-1-ol was made according to the same procedures as for (2R)-2-tetrahydropyran-2-yloxypropan-1-ol intermediate 131, starting from (2S)-2-hydroxypropanoate.

To a suspension of sodium hydride (60% dispersion in mineral oil) (127 mg, 3.18 mmol) in DMF (15 mL) at 0° C. was added (2S)-2-tetrahydropyran-2-yloxypropan-1-ol (509 mg, 3.18 mmol). The reaction mixture was stirred for 30 min at 0° C. and a solution of [(3R)-3-allyloxybutyl] 4-methylbenzenesulfonate (820 mg, 2.9 mmol) in DMF (14 mL) was added dropwise. The reaction mixture was stirred at 0° C. for 5 min then warmed up to RT and stirred overnight. The reaction mixture was quenched with a saturated aqueous ammonium chloride solution and poured in ethyl acetate. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford 2-[(1S)-2-[(3R)-3-allyloxybutoxy]-1-methyl-ethoxy]tetrahydropyran as a colorless oil.

¹H NMR (400 MHz, CDCl₃), 5.99-5.89 (1H, m), 5.28 (1H, dq, J=1.7, 6.8 Hz), 5.19-5.14 (1H, m), 4.83-4.73 (1H, m), 4.09-4.03 (1H, m), 4.00-3.91 (3H, m), 3.67-3.49 (4H, m), 3.40 (2H, tt, J=7.8, 8.4 Hz), 1.88-1.70 (4H, m), 1.63-1.50 (4H, m), 1.23 (3H, d, J=6.4 Hz), 1.19 (3H, d, J=6.0 Hz) ppm.

### Preparation of Intermediate 255: (2S)-1-[(3R)-3-allyloxybutoxy]propan-2-ol

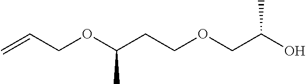

To 2-[(1S)-2-[(3R)-3-allyloxybutoxy]-1-methyl-ethoxy]tetrahydropyran (350 mg, 1.29 mmol) was added TFA (1 mL, 12.9 mmol). The reaction mixture was stirred at 20° C. for 6 h. The solvent was evaporated under reduced pressure and the residue was purified by silica gel column chromatography using cyclohexane/(ethyl acetate/ethanol (3/1)) 100/0 to 70/30 as eluent. to afford (2S)-1-[(3R)-3-allyloxybutoxy]propan-2-ol as a yellow oil.

¹H NMR (400 MHz, CDCl₃), 5.98-5.88 (1H, m), 5.32-5.25 (2H, m), 5.19-5.15 (1H, m), 4.09-4.03 (1H, m), 3.94-3.88 (1H, m), 3.59-3.54 (5H, m), 1.83-1.65 (2H, m), 1.45 (1H, s), 1.37 (3H, d, J=6.5 Hz), 1.18 (3H, d, J=6.3 Hz) ppm.

### Preparation of Intermediate 256: 3-[2-[(1S)-2-[(3R)-3-allyloxybutoxy]-1-methyl-ethoxy]pyrimidin-4-yl]-1-tetrahydropyran-2-yl-indazol-5-ol

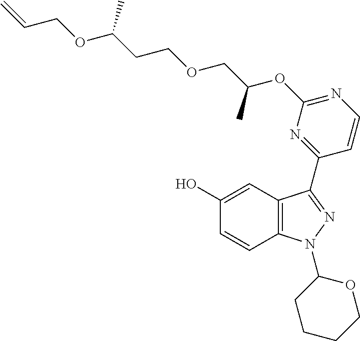 [SMILES: C=CCO[C@H](C)CCOC[C@H](C)Oc1nccc(-c2nn(C3CCCCO3)c3ccc(O)cc23)n1]

To a suspension of sodium hydride (60% dispersion in mineral oil) (33 mg, 0.83 mmol) in DMF (3.5 mL) at 0° C. was added (2S)-1-[(3R)-3-allyloxybutoxy]propan-2-ol (130 mg, 0.69 mmol). The reaction mixture was stirred at 0° C. for 30 min and a solution of tert-butyl-dimethyl-[3-(2-methylsulfonylpyrimidin-4-yl)-1-tetrahydropyran-2-yl-indazol-5-yl]oxy-silane (intermediate 63) (337 mg, 0.69 mmol) in DMF (3.5 mL) was added dropwise at 0° C. The reaction mixture was stirred at 0° C. for 5 min then warmed up to RT and stirred for 16 h. The reaction mixture was quenched with a saturated aqueous ammonium chloride solution and poured in ethyl acetate. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent to afford 3-[2-[(1S)-2-[(3R)-3-allyloxybutoxy]-1-methyl-ethoxy]pyrimidin-4-yl]-1-tetrahydropyran-2-yl-indazol-5-ol as a yellow gum.

LCMS method F: [M+H]⁺=483.3, t_{R}=2.92 min

### Preparation of Intermediate 258: 3-[2-[(1S)-2-[(3R)-3-hydroxybutoxy]-1-methyl-ethoxy]pyrimidin-4-yl]-1-tetrahydropyran-2-yl-indazol-5-ol

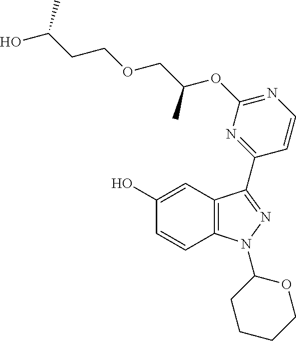 [SMILES: C[C@@H](O)CCOC[C@H](C)Oc1nccc(-c2nn(C3CCCCO3)c3ccc(O)cc23)n1]

To a solution of 3-[2-[(1S)-2-[(3R)-3-allyloxybutoxy]-1-methyl-ethoxy]pyrimidin-4-yl]-1-tetrahydropyran-2-yl-indazol-5-ol (160 mg, 0.33 mmol) in methanol (3.3 mL) was added tetrakis(triphenylphosphine)palladium(0) (19 mg, 0.017 mmol). The mixture was stirred at 20° C. for 5 min and 1,3-dimethylbarbituric acid (103 mg, 0.66 mmol) was added. The reaction mixture was stirred at 60° C. for 1 h. The solvent was evaporated under reduced pressure and the residue was purified by silica gel column chromatography using cyclohexane/(ethyl acetate/ethanol (3/1)) 100/0 to 50/50 as eluent to afford 3-[2-[(1S)-2-[(3R)-3-hydroxybutoxy]-1-methyl-ethoxy]pyrimidin-4-yl]-1-tetrahydropyran-2-yl-indazol-5-ol as a brown powder.

LCMS method F: [M+H]⁺=443.5, t_{R}=2.36 min

### Preparation of Intermediate 259: (8S,13S)-8,13-dimethyl-19-(oxan-2-yl)-7,10,14-trioxa-5,19,20,23-tetraazatetracyclo[13.5.2.1^{2,5}.0^{18,21}]tricosa-1(20),2(23),3,5,15(22), 16, 18(21)-heptaene

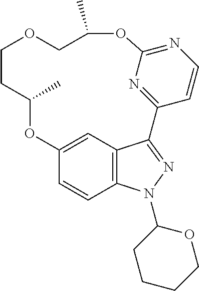 [SMILES: C[C@H]1CCOC[C@H](C)Oc2nccc(n2)-c2nn(C3CCCCO3)c3ccc(cc23)O1]

To a solution of diisopropyl azodicarboxylate (160 μL, 0.81 mmol) in THF (8 mL) at 60° C. was added a solution of triphenylphosphine (212 mg, 0.81 mmol) in toluene (10 mL) and a solution of 3-[2-[(1S)-2-[(3R)-3-hydroxybutoxy]-1-methyl-ethoxy]pyrimidin-4-yl]-1-tetrahydropyran-2-yl-indazol-5-ol (120 mg, 0.27 mmol) in THF (9 mL). The reaction mixture was stirred at 60° C. for 1 h. The solvent was evaporated under reduced pressure. The residue was partitioned between ethyl acetate and water. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford (8S,13S)-8,13-dimethyl-19-(oxan-2-yl)-7,10,14-trioxa-5,19,20,23-tetraazatetracyclo[13.5.2.1^{2,6}.0^{18,21}] tricosa-1(20),2(23),3,5,15(22),16,18(21)-heptaene as a white powder.

LCMS method F: [M+H]⁺=425.5, t_{R}=3.05 min

### Preparation of Example 46: (8S,13S)-8,13-dimethyl-7,10,14-trioxa-5,19,20,23-tetra azatetracyclo[13.5.2.1^{2,6}.0^{18,21}]tricosa-1(20),2(23),3,5,15(22),16,18(21)-heptaene

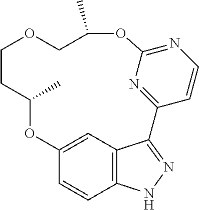 [SMILES: C[C@H]1CCOC[C@H](C)Oc2nccc(n2)-c2n[nH]c3ccc(cc23)O1]

To a solution of (8S,13S)-8,13-dimethyl-19-(oxan-2-yl)-7,10,14-trioxa-5,19,20,23-tetraaza tetracyclo[13.5.2.1^{2,6}.0^{18,21}]tricosa-1(20),2(23),3,5,15(22),16,18(21)-heptaene (240 mg, 0.2 mmol) in methanol (17.5 mL) and water (2.5 mL) was added p-toluenesulfonic acid monohydrate (190 mg, 1 mmol). The reaction mixture was stirred at 65° C. for 48 h. The solvent was evaporated under reduced pressure and the residue was neutralized by addition of a saturated aqueous NaHCO₃ solution. The aqueous layer was extracted with ethyl acetate. The combined organic layers were washed with brine, dried over anhydrous sodium sulfate, filtered and evaporated under reduced pressure. The residue was purified by preparative HPLC to afford (8S,13S)-8,13-dimethyl-7,10,14-trioxa-5,19,20,23-tetraazatetracyclo[13.5.2.1^{2,6}.0^{18,21}]tricosa-1(20),2(23),3,5,15(22),16,18(21)-heptaene as a solid.

LCMS method F: [M+H]⁺=341.2, t_{R}=2.38 min

LCMS method G: [M+H]⁺=341.2, t_{R}=2.32 min

¹H NMR (400 MHz, d6-DMSO) δ 13.66 (1H, s), 8.61 (1H, d, J=5.1 Hz), 8.36 (1H, d, J=2.3 Hz), 7.67 (1H, d, J=5.1 Hz), 7.54 (1H, d, J=8.7 Hz), 7.06 (1H, dd, J=2.5, 8.9 Hz), 5.28-5.23 (1H, m), 4.38-4.34 (1H, m), 4.05 (1H, dd, J=4.2, 9.9 Hz), 3.89-3.83 (1H, m), 3.74 (1H, dd, J=7.4, 9.9 Hz), 3.62-3.56 (1H, m), 2.21-2.16 (1H, m), 1.79-1.72 (1H, m), 1.48 (3H, d, J=6.6 Hz), 1.35 (3H, d, J=6.3 Hz) ppm.

### Example 47: (8S,13S)-8,13-dimethyl-7,10,14-trioxa-4,19,20,23-tetraazatetracyclo[13.5.2.1^{2,6}.0^{18,21}]tricosa-1(20),2(23),3,5,15(22),16,18(21)-heptaene

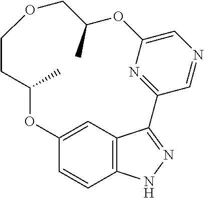 [SMILES: C[C@H]1CCOC[C@H](C)Oc2cncc(n2)-c2n[nH]c3ccc(cc23)O1]

Example 47 is prepared according to the synthesis route described in general Scheme D.

### Preparation of Intermediate 260: 2-[(1S)-2-[(3R)-3-benzyloxybutoxy]-1-methyl-ethoxy]-6-chloro-pyrazine

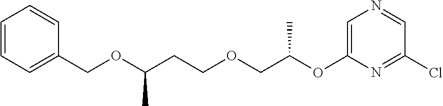 [SMILES: C[C@H](CCOC[C@H](C)Oc1cncc(Cl)n1)OCc1ccccc1]

(2S)-1-[(3R)-3-benzyloxybutoxy]propan-2-ol was prepared according to the same synthesis procedures as (2R)-1-[(3R)-3-benzyloxybutoxy]propan-2-ol intermediate 133 starting from (3R)-butane-1,3-diol and methyl (2S)-2-hydroxypropanoate.

To a solution of (2S)-1-[(3R)-3-benzyloxybutoxy]propan-2-ol (400 mg, 1.681 mmol) in dry THF (12 mL) at 0° C. was added sodium hydride (60% dispersion in mineral oil) (87 mg, 2.185 mmol). The reaction mixture was stirred at the 0° C. for 30 min then 2,6-dichloropyrazine (250 mg, 1.681 mmol) was added. The reaction mixture was stirred at 0° C. for 48 h then RT for 16 h. A saturated aqueous ammonium chloride solution was added and the aqueous layer was extracted with ethyl acetate. The combined organic layers were washed with brine, dried over anhydrous sodium sulfate, filtered and evaporated under reduced pressure to afford 2-[(1S)-2-[(3R)-3-benzyloxybutoxy]-1-methyl-ethoxy]-6-chloro-pyrazine as a pale yellow oil which was used in the next step without further purification.

LCMS method F: [M+H]⁺=351.2, t_{R}=3.19 min

### Preparation of Intermediate 261: [3-[6-[(1S)-2-[(3R)-3-benzyloxybutoxy]-1-methyl-ethoxy]pyrazin-2-yl]-1-tetrahydropyran-2-yl-indazol-5-yl]oxy-tert-butyl-dimethyl-silane

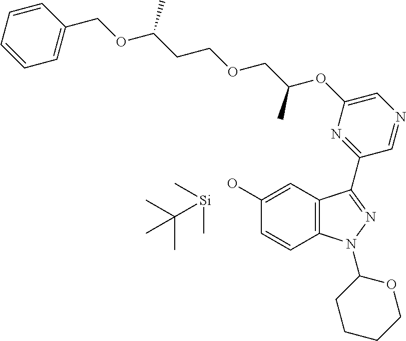

To a degassed solution of 2-[(1S)-2-[(3R)-3-benzyloxybutoxy]-1-methyl-ethoxy]-6-chloro-pyrazine (577 mg, 1.649 mmol), tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (906 mg, 1.979 mmol), potassium phosphate tribasic (1.05 g, 4.947 mmol) in dioxane (23 mL) and water (2.3 mL) was added tetrakis(triphenylphosphine)palladium(0) (95 mg, 0.082 mmol). The reaction mixture was stirred at 100° C. for 16 h. The reaction mixture was cooled to RT and diluted with water. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford [3-[6-[(1S)-2-[(3R)-3-benzyloxybutoxy]-1-methyl-ethoxy]pyrazin-2-yl]-1-tetrahydropyran-2-yl-indazol-5-yl]oxy-tert-butyl-dimethyl-silane as a pale yellow oil.

LCMS method I: [M+H]⁺=647.4, t_{R}=3.93 min

### Preparation of Intermediate 262: (2R)-4-[(2S)-2-[6-[5-[tert-butyl(dimethyl)silyl]oxy-1-tetrahydropyran-2-yl-indazol-3-yl]pyrazin-2-yl]oxypropoxy]butan-2-ol

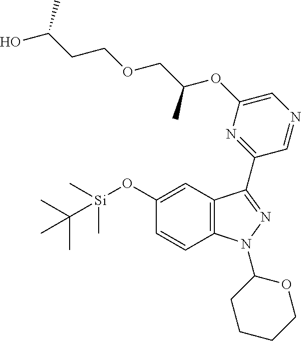 [SMILES: C[C@@H](O)CCOC[C@H](C)Oc1cncc(-c2nn(C3CCCCO3)c3ccc(O[Si](C)(C)C(C)(C)C)cc23)n1]

To a solution of [3-[6-[(1S)-2-[(3R)-3-benzyloxybutoxy]-1-methyl-ethoxy]pyrazin-2-yl]-1-tetrahydropyran-2-yl-indazol-5-yl]oxy-tert-butyl-dimethyl-silane (714 mg, 1.105 mmol) in dichloromethane (22 mL) and water (1.1 mL) at RT was added 2,3-dichloro-5,6-dicyano-p-benzoquinone (627 mg, 2.763 mmol). The reaction mixture was stirred at RT for 3 h then 40° C. for 2.5 h. The reaction mixture was quenched by saturated aqueous Na₂CO₃ solution and dichloromethane was added. The aqueous layer was extracted with dichloromethane and the combined organic layers were washed with water and brine, dried over anhydrous sodium sulfate, filtered and evaporated under reduced pressure. The residue was purified by silica gel column chromatography using cyclohexane/ethyl acetate 100/0 to 70/30 as eluent to afford (2R)-4-[(2S)-2-[6-[5-[tert-butyl(dimethyl)silyl]oxy-1-tetrahydropyran-2-yl-indazol-3-yl] pyrazin-2-yl]oxypropoxy]butan-2-ol as an orange oil.

LCMS method M: [M+H]⁺=557.3, t_{R}=4.46-4.56 min

### Preparation of Intermediate 263: [(1R)-3-[(2S)-2-[6-[5-[tert-butyl(dimethyl)silyl]oxy-1-tetrahydropyran-2-yl-indazol-3-yl]pyrazin-2-yl]oxypropoxy]-1-methyl-propyl] methanesulfonate

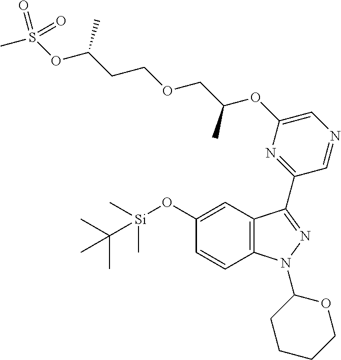 [SMILES: C[C@H](CCOC[C@H](C)Oc1cncc(-c2nn(C3CCCCO3)c3ccc(O[Si](C)(C)C(C)(C)C)cc23)n1)OS(C)(=O)=O]

To solution of (2R)-4-[(2S)-2-[6-[5-[tert-butyl(dimethyl)silyl]oxy-1-tetrahydropyran-2-yl-indazol-3-yl]pyrazin-2-yl]oxypropoxy]butan-2-ol (330 mg, 0.594 mmol) and triethylamine (165 μL, 1.188 mmol) in dry dichloromethane (13 mL) at 0° C. was added methanesulfonyl chloride (69 μL, 0.891 mmol). The reaction mixture was stirred at RT for 5 h. The reaction mixture was quenched with brine and extracted with dichloromethane. The combined organic layers were dried over anhydrous sodium sulfate, filtered and evaporated under reduced pressure to afford [(1R)-3-[(2S)-2-[6-[5-[tert-butyl(dimethyl)silyl]oxy-1-tetrahydropyran-2-yl-indazol-3-yl]pyrazin-2-yl]oxypropoxy]-1-methyl-propyl]methanesulfonate as an orange oil which was used in the next step without further purification.

LCMS method F: [M+H]⁺=635.3, t_{R}=5.82-5.88 min

### Preparation of Intermediate 264: (8S,13S)-8,13-dimethyl-19-(oxan-2-yl)-7,10,14-trioxa-4,19,20,23-tetraazatetracyclo[13.5.2.1^{2,6}.0^{18,21}]tricosa-1(20),2(23),3,5,15(22), 16,18(21)-heptaene

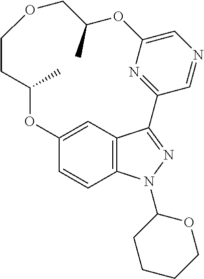 [SMILES: C[C@H]1CCOC[C@H](C)Oc2cncc(n2)-c2nn(C3CCCCO3)c3ccc(cc23)O1]

To a solution of sodium hydride (60% dispersion in mineral oil) (71 mg, 1.782 mmol) in dry DMF (100 mL) was added dropwise a solution of [(1R)-3-[(2S)-2-[6-[5-[tert-butyl(dimethyl) silyl]oxy-1-tetrahydropyran-2-yl-indazol-3-yl]pyrazin-2-yl]oxypropoxy]-1-methyl-propyl] methanesulfonate (377 mg, 0.594 mmol) in dry DMF (35 mL). The reaction mixture was stirred at RT for 16 h. The reaction mixture was quenched with water, concentrated under reduced pressure and diluted with water and ethyl acetate. The aqueous layer was extracted with ethyl acetate. The combined organic layers were washed with water then brine, dried over anhydrous sodium sulfate, filtered and evaporated under reduced pressure. The residue was purified by silica gel column chromatography using cyclohexane/ethyl acetate 100/0 to 70/30 as eluent to afford (8S,13S)-8,13-dimethyl-19-(oxan-2-yl)-7,10,14-trioxa-4,19,20,23-tetraazatetracyclo [13.5.2.1^{2,6}.0^{18,21}]tricosa-1(20),2(23),3,5,15(22),16,18(21)-heptaene as a white solid.

LCMS method F: [M+H]⁺=425.2, t_{R}=3.27 min

### Preparation of Example 47: (8S,13S)-8,13-dimethyl-7,10,14-trioxa-4,19,20,23-tetra azatetracyclo[13.5.2.1^{2,6}.0^{18,21}]tricosa-1(20),2(23),3,5,15(22),16,18(21)-heptaene

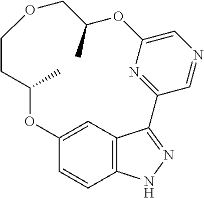 [SMILES: C[C@H]1CCOC[C@H](C)Oc2cncc(n2)-c2n[nH]c3ccc(cc23)O1]

To a solution of (8S,13S)-8,13-dimethyl-19-(oxan-2-yl)-7,10,14-trioxa-4,19,20,23-tetraaza tetracyclo[13.5.2.1^{2,6}.0^{18,21}]tricosa-1(20),2(23),3,5,15(22),16,18(21)-heptaene (120 mg, 0.283 mmol) in methanol (21 mL) and water (3 mL) at RT was added p-toluenesulfonic acid monohydrate (269 mg, 1.415 mmol). The reaction mixture was stirred at 65° C. for 16 h. The reaction mixture was concentrated under reduced pressure and a saturated aqueous Na₂CO₃ solution and ethyl acetate were added. The aqueous layer was extracted with ethyl acetate. The combined organic layers were washed with water and brine, dried over anhydrous sodium sulfate, filtered and evaporated under reduced pressure. The residue was triturated with diisopropyl ether, filtered, washed with diisopropyl ether and dried to afford (8S,13S)-8,13-dimethyl-7,10,14-trioxa-4,19,20,23-tetraazatetracyclo[13.5.2.1^{2,6}.0^{18,21}]tricosa-1(20),2(23),3, 5,15(22),16,18(21)-heptaene as a cream solid.

LCMS method F: [M+H]⁺=341.2, t_{R}=2.60 min

LCMS method G: [M+H]⁺=341.2, t_{R}=2.53 min

¹H NMR (400 MHz, d6-DMSO) δ 13.42 (1H, s), 8.85 (1H, m), 8.27 (1H, m), 8.19 (1H, s), 7.53-7.51 (1H, m), 7.05-7.02 (1H, dd, J=2.3, 9.0 Hz), 5.24-5.18 (1H, m), 4.44-4.39 (1H, m), 4.05-4.00 (1H, m), 3.85-3.79 (1H, m), 3.77-3.72 (1H, m), 3.63-3.56 (1H, m), 2.28-2.20 (1H, m), 1.76-1.67 (1H, m), 1.46 (3H, d, J=6.6 Hz), 1.34 (3H, d, J=6.3 Hz) ppm.

### Example 48: (12S)-12-methyl-9,13-dioxa-3-thia-18,19,22-triazatetracyclo[12.5.2.1^{2,5}.0^{17,20}]docosa-1(19),2(22),4,14(21),15,17(20)-hexaene

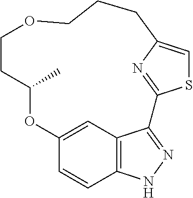 [SMILES: C[C@H]1CCOCCCc2csc(n2)-c2n[nH]c3ccc(cc23)O1]

Example 48 is prepared according to the synthesis route described in general Scheme D.

### Preparation of Intermediate 264 A: ethyl (Z/E)-3-(2-bromothiazol-4-yl)acrylate

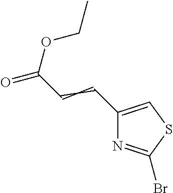

A solution of 2-bromo-4-formylthiazole (4.7 g, 24.475 mmol) and (carbethoxymethylene) triphenylphosphorane (10.232 g, 29.370 mmol) in toluene (73 mL) under nitrogen atmosphere was stirred at 100° C. for 2 h. The solvent was removed under reduced pressure and the residue was purified by silica gel column chromatography using heptane/ethyl acetate 100/0 to 95/5 as eluent to afford ethyl (Z/E)-3-(2-bromothiazol-4-yl)acrylate as a white solid.

LCMS method B: [M+H]⁺=263.8, t_{R}=0.848 min

### Preparation of Intermediate 265: 3-(2-bromothiazol-4-yl)propan-1-ol

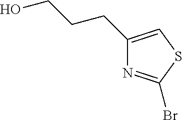

To a solution of ethyl (Z/E)-3-(2-bromothiazol-4-yl)acrylate (6.72 g, 25.636 mmol) in ethanol (128 mL) and THF (128 mL) at 0° C. was added sodium borohydride (3.879 g, 102.544 mmol) and calcium dichloride (5.691 g, 51.272 mmol). The reaction mixture was stirred at RT for 16 h. The reaction mixture was diluted with dichloromethane and saturated aqueous NaHCO₃ solution. The phases were separated and the aqueous layer was extracted with dichloromethane.

The combined organic layers were dried over anhydrous magnesium sulfate, filtered and evaporated under reduced pressure. The residue was purified by silica gel column chromatography using heptane/ethyl acetate 100/0 to 80/20 as eluent to afford 3-(2-bromothiazol-4-yl)propan-1-ol as a colorless oil.

LCMS method B: [M+H]⁺=221.9, t_{R}=0.421 min

### Preparation of Intermediate 268: (R)-4-(3-(3-(benzyloxy)butoxy)propyl)-2-bromo thiazole

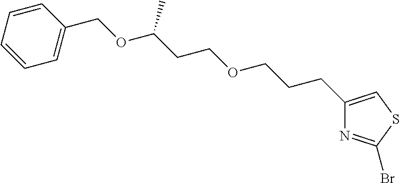 [SMILES: C[C@H](CCOCCCc1csc(Br)n1)OCc1ccccc1]

To a suspension of 3-(2-bromothiazol-4-yl)propan-1-ol (1.6 g, 7.204 mmol) and potassium iodide (1.196 g, 7.204 mmol) in dry DMF (18 mL) at 0° C. under nitrogen atmosphere was added sodium hydride (60% dispersion in mineral oil) (576 mg, 14.408 mmol). The reaction mixture was stirred at 0° C. for 30 min then (R)-(((4-bromobutan-2-yl)oxy)methyl)benzene (intermediate 166) (2.627 g, 10.806 mmol) in dry DMF (18 mL) was added. The reaction mixture was stirred at RT for 5 h. The reaction mixture was diluted with dichloromethane and water. The phases were separated and the aqueous layer was extracted with dichloromethane. The combined organic layers were washed with brine, dried over anhydrous magnesium sulfate, filtered and evaporated under reduced pressure. The residue was purified by silica gel column chromatography using heptane/ethyl acetate 100/0 to 93/7 as eluent to afford (R)-4-(3-(3-(benzyloxy)butoxy)propyl)-2-bromothiazole as a colorless oil.

LCMS method E: [M+H]⁺=384.0, t_{R}=4.49 min

### Preparation of Intermediate 269: [3-[4-[3-[(3R)-3-benzyloxybutoxy]propyl]thiazol-2-yl]-1-tetrahydropyran-2-yl-indazol-5-yl]oxy-tert-butyl-dimethyl-silane

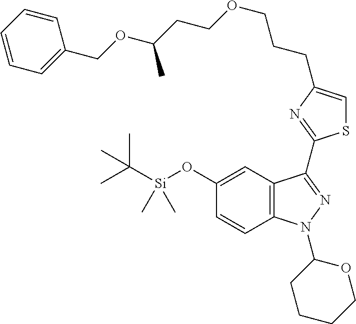 [SMILES: C[C@H](CCOCCCc1csc(-c2nn(C3CCCCO3)c3ccc(O[Si](C)(C)C(C)(C)C)cc23)n1)OCc1ccccc1]

To a degassed solution of tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (1 g, 2.18 mmol), 4-[3-[(3R)-3-benzyloxybutoxy]propyl]-2-bromo-thiazole (1.07 g, 2.78 mmol) and potassium phosphate tribasic (1.386 g, 6.54 mmol) in dioxane (22 mL) and water (0.5 mL) were added tetrakis(triphenylphosphine)palladium(0) (127 mg, 0.11 mmol) and 2-dicyclohexylphosphino-2′,4′,6′-triisopropylbiphenyl (105 mg, 0.22 mmol). The reaction mixture was stirred at 130° C. for 3 h. The reaction mixture was filtered through a pad of Celite and washed with ethyl acetate.

The filtrate was diluted with water and extracted with ethyl acetate. The combined organic layers were washed with water then brine, dried over anhydrous magnesium sulfate, filtered and evaporated under reduced pressure. The residue was purified by silica gel column chromatography using cyclohexane/ethyl acetate 100/0 to 90/10 as eluent to afford [3-[4-[3-[(3R)-3-benzyloxybutoxy]propyl]thiazol-2-yl]-1-tetrahydropyran-2-yl-indazol-5-yl]oxy-tert-butyl-dimethyl-silane as a yellow oil.

LCMS method J: [M+H]⁺=636.3, t_{R}=6.66 min

### Preparation of Intermediate 270: (2R)-4-[3-[2-[5-[tert-butyl(dimethyl)silyl]oxy-1-tetrahydropyran-2-yl-indazol-3-yl]thiazol-4-yl]propoxy]butan-2-ol

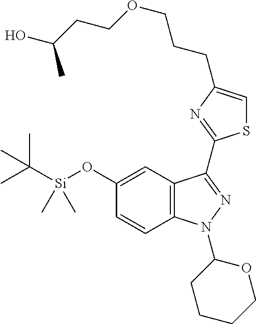 [SMILES: C[C@@H](O)CCOCCCc1csc(-c2nn(C3CCCCO3)c3ccc(O[Si](C)(C)C(C)(C)C)cc23)n1]

To a solution of [3-[4-[3-[(3R)-3-benzyloxybutoxy]propyl]thiazol-2-yl]-1-tetrahydropyran-2-yl-indazol-5-yl]oxy-tert-butyl-dimethyl-silane (1.632 g, 2.57 mmol) in ethyl acetate (25 mL) at RT was added palladium hydroxide on carbon (360 mg, 20% wt loading). The reaction mixture was stirred under hydrogen atmosphere at RT overnight. Additional palladium hydroxide on carbon (0.376 g) was added and the reaction mixture was stirred at 40° C. for 8 h. Additional hydroxide on carbon (0.376 g) was added and the reaction mixture was stirred at 50° C. for 16 h. The reaction mixture was heated up to 70° C. for 96 h. Additional palladium hydroxide on carbon (0.376 g) was added and the reaction mixture was stirred at 80° C. for 24 h. The reaction mixture was filtered, washed with ethyl acetate and the filtrate was concentrated under reduced pressure. The residue was purified by silica gel column chromatography using dichloromethane/methanol 100/0 to 95:5 as eluent to afford (2R)-4-[3-[2-[5-[tert-butyl (dimethyl)silyl]oxy-1-tetrahydropyran-2-yl-indazol-3-yl]thiazol-4-yl]propoxy]butan-2-ol as a yellow oil.

LCMS method J: [M+H]⁺=546.3, t_{R}=5.84 min

### Preparation of Intermediate 271: [(1R)-3-[3-[2-[5-[tert-butyl(dimethyl)silyl]oxy-1-tetrahydropyran-2-yl-indazol-3-yl]thiazol-4-yl]propoxy]-1-methyl-propyl]methane sulfonate

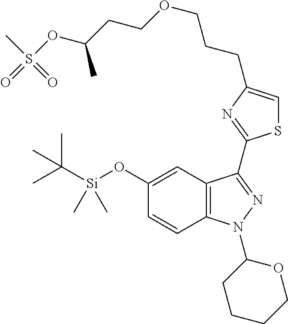 [SMILES: C[C@H](CCOCCCc1csc(-c2nn(C3CCCCO3)c3ccc(O[Si](C)(C)C(C)(C)C)cc23)n1)OS(C)(=O)=O]

To a stirred suspension of (2R)-4-[3-[2-[5-[tert-butyl(dimethyl)silyl]oxy-1-tetrahydropyran-2-yl-indazol-3-yl]thiazol-4-yl]propoxy]butan-2-ol (230 mg, 0.42 mmol) in dichloromethane (12 mL) at RT under argon atmosphere was added triethylamine (348 μL, 2.52 mmol) followed by methanesulfonyl chloride (97 μL, 1.26 mmol). The reaction mixture was stirred at RT for 1 h. The reaction mixture was washed with a saturated aqueous NaHCO₃ solution and brine, dried over anhydrous magnesium sulfate, filtered and evaporated under reduced pressure to afford [(1R)-3-[3-[2-[5-[tert-butyl(dimethyl)silyl]oxy-1-tetrahydropyran-2-yl-indazol-3-yl]thiazol-4-yl]propoxy]-1-methyl-propyl] methanesulfonate as a yellow oil which was used in the next step without further purification.

LCMS method J: [M+H]⁺=624.3, t_{R}=5.99 min

### Preparation of Intermediate 272: [(1R)-3-[3-[2-(5-hydroxy-1-tetrahydropyran-2-yl-indazol-3-yl)thiazol-4-yl]propoxy]-1-methyl-propyl] methanesulfonate

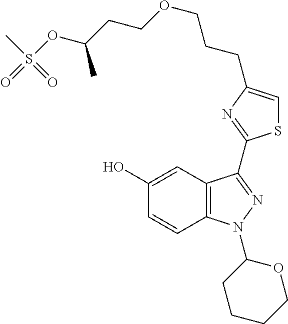 [SMILES: C[C@H](CCOCCCc1csc(-c2nn(C3CCCCO3)c3ccc(O)cc23)n1)OS(C)(=O)=O]

To a solution of [(1R)-3-[3-[2-[5-[tert-butyl(dimethyl)silyl]oxy-1-tetrahydropyran-2-yl-indazol-3-yl]thiazol-4-yl]propoxy]-1-methyl-propyl] methanesulfonate (205 mg, 0.32 mmol) in THF (3.5 mL) at RT was added TBAF (1M solution in THF) (360 μL, 0.36 mmol). The reaction mixture was stirred at RT for 2 h. The reaction mixture was diluted with a saturated aqueous ammonium chloride solution and extracted with ethyl acetate. The combined organic layers were washed with brine, dried over anhydrous magnesium sulfate, filtered and evaporated under reduced pressure. The residue was purified by silica gel column chromatography using cyclohexane/ethyl acetate 100/0 to 60/40 as eluent to afford [(1R)-3-[3-[2-(5-hydroxy-1-tetrahydropyran-2-yl-indazol-3-yl)thiazol-4-yl]propoxy]-1-methyl-propyl] methanesulfonate as a colorless oil.

LCMS method F: [M+H]⁺=510.2, t_{R}=2.81 min

### Preparation of Intermediate 273: (12S)-12-methyl-18-(oxan-2-yl)-9,13-dioxa-3-thia-18,19,22-triazatetracyclo[12.5.2.1^{2,5}.0^{17,20}]docosa-1(19),2(22),4,14(21),15,17(20)-hexaene

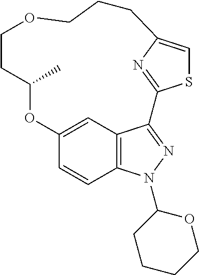 [SMILES: C[C@H]1CCOCCCc2csc(n2)-c2nn(C3CCCCO3)c3ccc(cc23)O1]

To a suspension of cesium carbonate (29 mg, 0.088 mmol) in anhydrous DMF (15 mL) at 80° C. was added dropwise [(1R)-3-[3-[2-(5-hydroxy-1-tetrahydropyran-2-yl-indazol-3-yl)thiazol-4-yl]propoxy]-1-methyl-propyl] methanesulfonate (15 mg, 0.029 mmol) in DMF (5 mL). The reaction mixture was stirred at 80° C. for 1 h. The reaction was allowed to cool down to RT, filtered, washed with ethyl acetate and the solvent was removed under reduced pressure. The crude product was purified by preparative TLC using dichloromethane/MeOH: (95/5) to afford (12S)-12-methyl-18-(oxan-2-yl)-9,13-dioxa-3-thia-18,19,22-triazatetracyclo[12.5.2.1^{2,5}.0^{17,20}] docosa-1(19),2(22),4,14(21),15,17(20)-hexaene as a colorless oil.

LCMS method J: [M+H]⁺=414.2, t_{R}=5.20 min

### Preparation of Example 48: (12S)-12-methyl-9,13-dioxa-3-thia-18,19,22-triazatetra cyclo[12.5.2.1^{2,5}.0^{17,20}]docosa-1(19),2(22),4,14(21),15,17(20)-hexaene

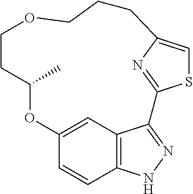 [SMILES: C[C@H]1CCOCCCc2csc(n2)-c2n[nH]c3ccc(cc23)O1]

To a solution of (12S)-12-methyl-18-(oxan-2-yl)-9,13-dioxa-3-thia-18,19,22-triazatetracyclo [12.5.2.1^{2,5}.0^{17,20}]docosa-1(19),2(22),4,14(21),15,17(20)-hexaene (15 mg, 0.036 mmol) in methanol (2.1 mL) and water (0.25 mL) was added p-toluenesulfonic acid monohydrate (35 mg, 0.18 mmol). The reaction mixture was stirred at 65° C. for 5 h. The reaction mixture was neutralized by addition of a saturated aqueous NaHCO₃ solution and the solvent was removed under reduced pressure. The residue was diluted with ethyl acetate and the aqueous layer was extracted with ethyl acetate. The combined organic layers were washed with brine, dried over anhydrous magnesium sulfate, filtered and evaporated under reduced pressure. The residue was purified by preparative-TLC eluting with cyclohexane/(ethyl acetate/ethanol (3/1)) 50/50 to afford (12S)-12-methyl-9,13-dioxa-3-thia-18,19,22-triazatetracyclo[12.5.2.1^{2,5}.0^{17,20}]docosa-1(19),2(22),4,14(21),15,17(20)-hexaene as a solid.

LCMS method F: [M+H]⁺=330.2, t_{R}=2.70 min

LCMS method G: [M+H]⁺=330.2, t_{R}=2.67 min

¹H NMR (400 MHz, CDCl₃) δ 13.42 (1H, s), 8.34 (1H, d, J=2.3 Hz), 7.41-7.38 (1H, m), 7.11 (1H, dd, J=2.5, 8.9 Hz), 6.91 (1H, d, =1.1 Hz), 4.89-4.80 (1H, m), 4.60-4.53 (1H, m), 4.13-3.91 (1H, bs), 3.85 (1H, dt, J=9.8, 2.8 Hz), 3.76-3.65 (2H, m), 3.53-3.46 (1H, m), 3.13-3.06 (1H, m), 2.77-2.58 (2H, m), 2.42-2.28 (1H, m), 2.23-2.14 (1H, m), 1.47 (3H, d, J=6.4 Hz) ppm.

### Example 49: (8R,13S)-4-[(3R)-3-methoxypyrrolidin-1-yl]-8,13-dimethyl-7,10,14-trioxa-5,19,20,23-tetraazatetracyclo[13.5.2.1^{2,6}.0^{18,21}]tricosa-1(20),2(23),3,5, 15(22),16,18(21)-heptaene

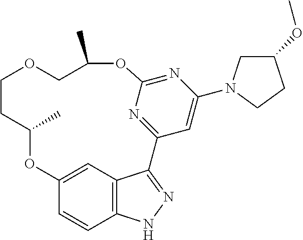 [SMILES: CO[C@@H]1CCN(c2cc3nc(n2)O[C@H](C)COCC[C@H](C)Oc2ccc4[nH]nc-3c4c2)C1]

Example 49 is prepared according to the synthesis route described in general Scheme C, following the same synthesis procedures as for Example 44. A Mitsunobu reaction is used for the macrocyclization step.

### Preparation of Intermediate 274: (8R,13S)-4-[(3R)-3-methoxypyrrolidin-1-yl]-8,13-dimethyl-19-(oxan-2-yl)-7,10,14-trioxa-5,19,20,23-tetraazatetracyclo[13.5.2.1^{2,6}.0^{18,21}]tricosa-1(20),2(23),3,5,15(22),16,18(21)-heptaene

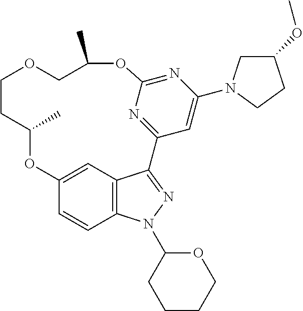 [SMILES: CO[C@@H]1CCN(c2cc3nc(n2)O[C@H](C)COCC[C@H](C)Oc2ccc4c(c2)c-3nn4C2CCCCO2)C1]

To a solution of diisopropyl azodicarboxylate (220 μL, 1.11 mmol) in THF (8 mL) at 60° C. was added a solution of triphenylphosphine (291 mg, 1.11 mmol) in toluene (20 mL) and a solution of 3-[2-[(1R)-2-[(3R)-3-hydroxybutoxy]-1-methyl-ethoxy]-6-[(3R)-3-methoxypyrrolidin-1-yl]pyrimidin-4-yl]-1-tetrahydropyran-2-yl-indazol-5-ol (200 mg, 0.37 mmol) in THF (9 mL).

The reaction mixture was stirred at 60° C. for 15 min. The solvent was removed under reduced pressure. The residue was purified by silica gel column chromatography using cyclohexane/(ethyl acetate/ethanol (3:1) 100/0 to 60/40 as eluent to afford (8R,13S)-4-[(3R)-3-methoxypyrrolidin-1-yl]-8,13-dimethyl-19-(oxan-2-yl)-7,10,14-trioxa-5,19,20,23-tetraazatetra cyclo[13.5.2.1^{2,6}.0^{18,21}]tricosa-1(20),2(23),3,5,15(22),16,18(21)-heptaene as a colorless oil.

LCMS method F: [M+H]⁺=524.4, t_{R}=3.38 min

### Preparation of Example 49: (8R,13S)-4-[(3R)-3-methoxypyrrolidin-1-yl]-8,13-dimethyl-7,10,14-trioxa-5,19,20,23-tetraazatetracyclo[13.5.2.1^{2,6}.0^{18,21}]tricosa-1(20), 2(23),3,5,15(22),16,18(21)-heptaene

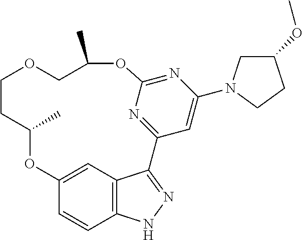 [SMILES: CO[C@@H]1CCN(c2cc3nc(n2)O[C@H](C)COCC[C@H](C)Oc2ccc4[nH]nc-3c4c2)C1]

To a solution of (8R,13S)-4-[(3R)-3-methoxypyrrolidin-1-yl]-8,13-dimethyl-19-(oxan-2-yl)-7,10,14-trioxa-5,19,20,23-tetraazatetracyclo[13.5.2.1^{2,6}.0^{18,21}]tricosa-1(20),2(23),3,5,15(22), 16,18(21)-heptaene (220 mg, 0.13 mmol) in methanol (11.5 mL) and water (1.5 mL) was added p-toluenesulfonic acid monohydrate (124 mg, 0.65 mmol). The reaction mixture was stirred at 60° C. for 48 h. The solvent was evaporated under reduced pressure and the residue was neutralized by addition of a saturated aqueous NaHCO₃ solution. The mixture was diluted with ethyl acetate. The aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent afford (8R,13S)-4-[(3R)-3-methoxypyrrolidin-1-yl]-8,13-dimethyl-7,10,14-trioxa-5,19,20,23-tetraazatetracyclo[13.5.2.1^{2,6}.0^{18,21}]tricosa-1(20),2(23),3,5,15(22),16,18(21)-heptaene as a solid.

LCMS method J: [M+H]⁺=440.5, t_{R}=3.89 min

LCMS method N: [M+H]⁺=440.5, t_{R}=3.93 min

¹H NMR (400 MHz, d6-DMSO) δ 13.38 (1H, s), 8.06 (1H, d, J=2.3 Hz), 7.49 (1H, d, J=8.9 Hz), 6.99 (1H, dd, J=2.3, 8.9 Hz), 6.84 (1H, s), 5.15-5.08 (1H, m), 4.67-4.59 (1H, m), 4.25 (1H, dd, J=3.3, 7.3 Hz), 4.08-4.08 (1H, m), 3.79-3.73 (1H, m), 3.65 (1H, s), 3.60-3.55 (3H, m), 3.46-3.39 (2H, m), 3.28 (3H, s), 3.27 (1H, dd, J=7.7, 9.1 Hz), 2.38-2.32 (1H, m), 2.06 (2H, s), 1.43 (3H, d, J=6.3 Hz), 1.37 (3H, d, J=6.1 Hz) ppm.

### Example 50: (13R)-13-methyl-8,11,14-trioxa-4,5,19,20-tetraazatetracyclo[13.5.2.1^{2,5}.0^{18,21}]tricosa-1(20),2(23),3,15(22),16,18(21)-hexaene

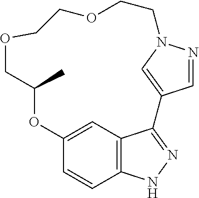 [SMILES: C[C@@H]1COCCOCCn2cc(cn2)-c2n[nH]c3ccc(cc23)O1]

Example 50 is prepared according to the synthesis route described in general Scheme D, following the same synthesis procedures as for Example 24.

### Preparation of Intermediate 275: (13R)-13-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene

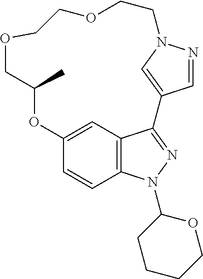 [SMILES: C[C@@H]1COCCOCCn2cc(cn2)-c2nn(C3CCCCO3)c3ccc(cc23)O1]

To a suspension of cesium carbonate (1.267 g, 3.90 mmol) in anhydrous DMF (70 mL) at 60° C. was added dropwise [(1S)-2-[2-[2-[4-[5-[tert-butyl(dimethyl)silyl]oxy-1-tetrahydropyran-2-yl-indazol-3-yl]pyrazol-1-yl]ethoxy]ethoxy]-1-methyl-ethyl] methanesulfonate (809 mg, 1.3 mmol) in DMF (30 mL). The reaction mixture was stirred at 60° C. for 16 h. The reaction mixture was filtered over a pad of Celite and washed with ethyl acetate. The filtrate was concentrated under reduced pressure. The residue was purified by silica gel column chromatography using cyclohexane/ethyl acetate 100/0 to 30/70 as eluent. to afford (13R)-13-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene as an orange solid.

LCMS method J: [M+H]⁺=413.2, t_{R}=3.69 min

### Preparation of Example 50: (13R)-13-methyl-8,11,14-trioxa-4,5,19,20-tetraazatetra cyclo[13.5.2.1^{2,5}.0^{18,21}]tricosa-1(20),2(23),3,15(22),16,18(21)-hexaene

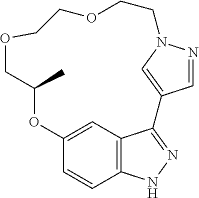 [SMILES: C[C@@H]1COCCOCCn2cc(cn2)-c2n[nH]c3ccc(cc23)O1]

To a solution of (13R)-13-methyl-19-(oxan-2-yl)-8,11,14-trioxa-4,5,19,20-tetraazatetracyclo [13.5.2.1^{2,5}.0^{18,21}]tricosa-1(20),2(23),3,15(22),16,18(21)-hexaene (300 mg, 0.73 mmol) in dichloromethane (4.3 mL) at RT was added TFA (1.08 mL, 14.6 mmol). The reaction mixture was stirred at RT for 16 h. The solvent was evaporated under reduced pressure and the residue was diluted with saturated NaHCO₃ solution and extracted twice with ethyl acetate. The combined organic layers were dried over anhydrous sodium sulfate, filtered and evaporated under reduced pressure. The residue was purified by silica gel column chromatography using dichloromethane/methanol 100/ to 97/3 as eluent to afford pure (13R)-13-methyl-8,11,14-trioxa-4,5,19,20-tetraazatetracyclo[13.5.2.1^{2,5}.0^{18,21}]tricosa-1(20),2(23),3,15(22),16,18(21)-hexaene as foam.

LCMS method F: [M+H]⁺=329.2, t_{R}=1.98 min

LCMS method G: [M+H]⁺=329.2, t_{R}=1.98 min

¹H NMR (400 MHz, CDCl₃) δ 8.55 (1H, s), 8.03 (1H, s), 7.96 (1H, d, J=2.4 Hz), 7.30 (1H, d, J=8.8 Hz), 7.05 (1H, dd, J=2.4, 8.8 Hz), 4.50-4.45 (3H, m), 4.04-3.96 (1H, m), 3.86 (1H, dd, J=5.2, 10.4 Hz), 3.80-3.76 (4H, m), 3.66-3.61 (1H, m), 3.55 (1H, dd, J=3.2, 10.8 Hz), 1.43 (3H, d, J=6.6 Hz) ppm.

### Example 51: (9S,13S)-9,13-dimethyl-7,10,14-trioxa-4,19,20,23-tetraazatetracyclo[13.5.2.1^{2,6}.0^{18,21}]tricosa-1(20),2(23),3,5,15(22),16,18(21)-heptaene

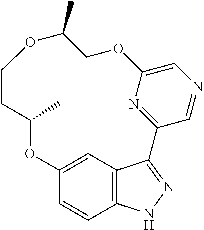 [SMILES: C[C@H]1COc2cncc(n2)-c2n[nH]c3ccc(cc23)O[C@@H](C)CCO1]

Example 51 is prepared according to the synthesis route described in general Scheme D.

### Preparation of Intermediate 276: (2S)-1-tetrahydropyran-2-yloxypropan-2-ol

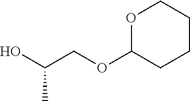

To a solution of (S)-(−)-1,2-propanediol (3 g, 39.484 mmol) in dry dichloromethane (60 mL) at RT was added 3,4-dihydro-2H-pyran (3.73 mL, 40.866 mmol) and pyridinium p-toluenesulfonate (119 mg, 0.474 mmol). The reaction mixture was stirred at RT for 16 h. A saturated aqueous NaHCO₃ solution was added and the mixture was extracted with dichloromethane. The combined organic layers were washed with brine, dried over anhydrous sodium sulfate, filtered and evaporated under reduced pressure. The residue was purified by silica gel column chromatography using cyclohexane/ethyl acetate 100/0 to 80/20 to afford (2S)-1-tetrahydropyran-2-yloxypropan-2-ol as a colorless liquid.

¹H NMR (400 MHz, DMSO) 4.56 (2H, m), 3.79-3.69 (2H, m), 3.51-3.36 (2H, m), 3.26-3.14 (1H, m), 1.79-1.55 (2H, m), 1.52-1.37 (4H, m), 1.06-1.02 (3H, m) ppm.

### Preparation of Intermediate 277: [(3R)-3-benzyloxybutyl]methanesulfonate

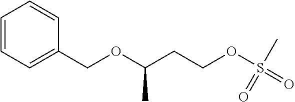 [SMILES: C[C@H](CCOS(C)(=O)=O)OCc1ccccc1]

To solution of (3R)-3-benzyloxybutan-1-ol (intermediate 128) (4.04 g, 22.44 mmol) and triethylamine (6.3 mL, 44.88 mmol) in dry dichloromethane (150 mL) at 0° C. was added dropwise methanesulfonyl chloride (2.6 mL, 33.66 mmol). The reaction mixture was stirred at RT for 16 h. The reaction mixture was quenched by water and the aqueous layer was extracted with dichloromethane. The combined organic layers were washed with brine, dried over anhydrous sodium sulfate, filtered and evaporated under reduced pressure to afford [(3R)-3-benzyloxybutyl]methanesulfonate as a yellow oil which was used in the next step without further purification.

LCMS method F: [M+H]⁺=259.1, t_{R}=2.44 min

### Preparation of Intermediate 278: 2-[(2S)-2-[(3R)-3-benzyloxybutoxy]propoxy]tetra hydropyran

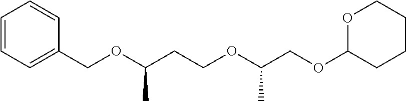 [SMILES: C[C@H](CCO[C@@H](C)COC1CCCCO1)OCc1ccccc1]

To a solution of (2S)-1-tetrahydropyran-2-yloxypropan-2-ol (intermediate 276) (2.52 g, 15.75 mmol) in dry DMF (100 mL) at 0° C. was added portionwise sodium hydride (60% dispersion in mineral oil) (945 mg, 23.634 mmol). The reaction mixture was stirred at 0° C. for 45 min and a solution of [(3R)-3-benzyloxybutyl] methanesulfonate (intermediate 277) (5.79 g, 22.442 mmol) in dry DMF (40 mL) was added dropwise. The reaction mixture was allowed to warmed up to RT and was stirred at 60° C. for 17 h. The reaction mixture was cooled to RT and quenched with water. ethyl acetate was added and the aqueous layer was extracted with ethyl acetate. The combined organic layers were washed with water and brine, dried over anhydrous sodium sulfate, filtered and evaporated under reduced pressure. The residue was purified by silica gel column chromatography using cyclohexane/ethyl acetate 100/0 to 80/20 as eluent. to afford 2-[(2S)-2-[(3R)-3-benzyloxybutoxy]propoxy]tetrahydropyran as a colorless oil.

LCMS method F: [M+Na]⁺=345.2, t_{R}=3.08 min

### Preparation of Intermediate 279: (2S)-2-[(3R)-3-benzyloxybutoxy]propan-1-ol

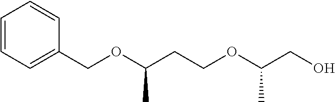 [SMILES: C[C@H](CCO[C@@H](C)CO)OCc1ccccc1]

To a solution of 2-[(2S)-2-[(3R)-3-benzyloxybutoxy]propoxy]tetrahydropyran (3.03 g, 9.41 mmol) in methanol (111 mL) and water (22.2 mL) was added p-toluenesulfonic acid monohydrate (8.95 g, 47.06 mmol) and the reaction mixture was stirred at 65° C. for 16 h. The reaction mixture was concentrated under reduced pressure and the residue was neutralized by addition of a saturated aqueous NaHCO₃ solution. The aqueous layer was extracted with ethyl acetate. The combined organic layers were washed with brine, dried over anhydrous sodium sulfate, filtered and evaporated under reduced pressure to afford (2S)-2-[(3R)-3-benzyloxybutoxy]propan-1-ol as a colorless oil which was used in the next step without further purification.

LCMS method J: [M+H]⁺=239.3, t_{R}=2.96 min

### Preparation of Intermediate 280: 2-[(2S)-2-[(3R)-3-benzyloxybutoxy]propoxy]-6-chloro-pyrazine

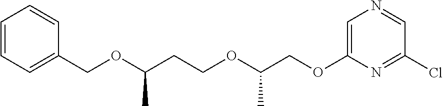 [SMILES: C[C@H](CCO[C@@H](C)COc1cncc(Cl)n1)OCc1ccccc1]

To a solution of (2S)-2-[(3R)-3-benzyloxybutoxy]propan-1-ol (400 mg, 1.681 mmol) in dry THF (12 mL) at 0° C. was added sodium hydride (60% dispersion in mineral oil) (87 mg, 2.18 mmol). The reaction mixture was stirred at 0° C. for 30 min then 2,6-dichloropyrazine (250 mg, 1.68 mmol) was added. The reaction mixture was stirred at 0° C. for 2 h then RT for 14 h. A saturated aqeous ammonium chloride solution was added and the aqueous layer was extracted with ethyl acetate. The combined organic layers were washed with brine, dried over anhydrous sodium sulfate, filtered and evaporated under reduced pressure to afford 2-[(2S)-2-[(3R)-3-benzyloxybutoxy]propoxy]-6-chloro-pyrazine as a yellow liquid. The product was used in the next step without further purification.

LCMS method L: [M+H]⁺=351, t_{R}=3.15 min

### Preparation of Intermediate 281: [3-[6-[(2S)-2-[(3R)-3-benzyloxybutoxy]propoxy] pyrazin-2-yl]-1-tetrahydropyran-2-yl-indazol-5-yl]oxy-tert-butyl-dimethyl-silane

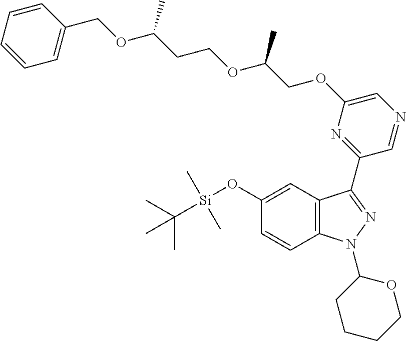 [SMILES: C[C@H](CCO[C@@H](C)COc1cncc(-c2nn(C3CCCCO3)c3ccc(O[Si](C)(C)C(C)(C)C)cc23)n1)OCc1ccccc1]

To a degassed solution of 2-[(2S)-2-[(3R)-3-benzyloxybutoxy]propoxy]-6-chloro-pyrazine (587 mg, 1.67 mmol), tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (921 mg, 2.01 mmol), potassium phosphate tribasic (1.06 g, 5.03 mmol) in dioxane (23 mL) and water (2.3 mL) was added tetrakis(triphenylphosphine)palladium(0) (97 mg, 0.084 mmol). The reaction mixture was stirred at 100° C. for 16 h then cooled to RT and diluted with water. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford [3-[6-[(2S)-2-[(3R)-3-benzyloxybutoxy]propoxy]pyrazin-2-yl]-1-tetrahydropyran-2-yl-indazol-5-yl]oxy-tert-butyl-dimethyl-silane as a pale yellow oil.

LCMS method M: [M+H]⁺=647.5, t_{R}=6.13 min

### Preparation of Intermediate 282: (2R)-4-[(1S)-2-[6-[5-[tert-butyl(dimethyl)silyl]oxy-1-tetrahydropyran-2-yl-indazol-3-yl]pyrazin-2-yl]oxy-1-methyl-ethoxy]butan-2-ol

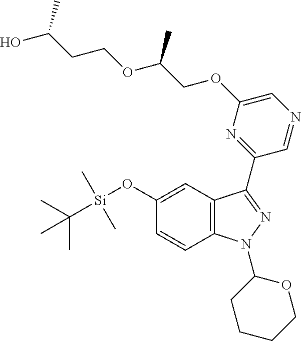 [SMILES: C[C@@H](O)CCO[C@@H](C)COc1cncc(-c2nn(C3CCCCO3)c3ccc(O[Si](C)(C)C(C)(C)C)cc23)n1]

To a solution of [3-[6-[(2S)-2-[(3R)-3-benzyloxybutoxy]propoxy]pyrazin-2-yl]-1-tetrahydro pyran-2-yl-indazol-5-yl]oxy-tert-butyl-dimethyl-silane (834 mg, 1.37 mmol) in dichloro methane (25.7 mL) in pH 7 phosphate buffer (1.3 mL) was added 2,3-dichloro-5,6-dicyano-p-benzoquinone (733 mg, 3.22 mmol) at RT. The reaction mixture was stirred at RT for 18 h. The reaction was quenched with a saturated aqueous Na₂CO₃ solution and ethyl acetate was added. The aqueous layer was extracted with ethyl acetate and the combined organic layers were washed with water and brine, dried over anhydrous sodium sulfate, filtered and evaporated under reduced pressure. The residue was purified by silica gel column chromatography using cyclohexane/ethyl acetate 100/0 to 60/40 as eluent to afford (2R)-4-[(1S)-2-[6-[5-[tert-butyl(dimethyl)silyl]oxy-1-tetrahydropyran-2-yl-indazol-3-yl]pyrazin-2-yl]oxy-1-methyl-ethoxy]butan-2-ol as an orange oil.

LCMS method L: [M+H]⁺=557.5, t_{R}=3.72 min

### Preparation of Intermediate 283: [(1R)-3-[(1S)-2-[6-[5-[tert-butyl(dimethyl)silyl]oxy-1-tetrahydropyran-2-yl-indazol-3-yl]pyrazin-2-yl]oxy-1-methyl-ethoxy]-1-methyl-propyl]methanesulfonate

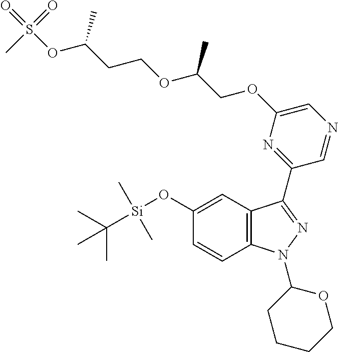 [SMILES: C[C@H](CCO[C@@H](C)COc1cncc(-c2nn(C3CCCCO3)c3ccc(O[Si](C)(C)C(C)(C)C)cc23)n1)OS(C)(=O)=O]

To a solution of (2R)-4-[(1S)-2-[6-[5-[tert-butyl(dimethyl)silyl]oxy-1-tetrahydropyran-2-yl-indazol-3-yl]pyrazin-2-yl]oxy-1-methyl-ethoxy]butan-2-ol (310 mg, 0.558 mmol) and triethylamine (156 μL, 1.116 mmol) in dry dichloromethane (12 mL) at 0° C. was added methanesulfonyl chloride (65 μL, 0.837 mmol). The reaction mixture was stirred at RT for 16 h. Additional methanesulfonyl chloride (10 μL, 0.112 mmol, 0.2 eq) was added and the reaction mixture was stirred at RT for 48 h. Additional triethylamine (156 μL, 1.116 mmol) and methanesulfonyl chloride (65 μL, 0.837 mmol) were added. The reaction mixture was stirred at RT for 5 h. The reaction mixture was quenched with brine and extracted with dichloromethane. The combined organic layers were dried over anhydrous sodium sulfate, filtered and concentrated under reduced pressure to afford [(1R)-3-[(1S)-2-[6-[5-[tert-butyl(dimethyl)silyl]oxy-1-tetrahydropyran-2-yl-indazol-3-yl]pyrazin-2-yl]oxy-1-methyl-ethoxy]-1-methyl-propyl]methanesulfonate as an orange oil which was used in the next step without further purification.

LCMS method J: [M+H]⁺=635.4, t_{R}=5.87 min

### Preparation of Intermediate 284: (9S,13S)-9,13-dimethyl-19-(oxan-2-yl)-7,10,14-trioxa-4,19,20,23-tetraazatetracyclo[13.5.2.1^{2,6}.0^{18,21}]tricosa-1(20),2(23),3, 5,15(22),16, 18(21)-heptaene

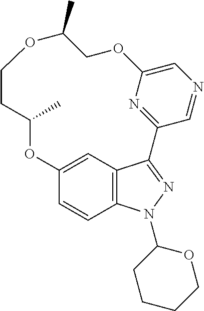 [SMILES: C[C@H]1COc2cncc(n2)-c2nn(C3CCCCO3)c3ccc(cc23)O[C@@H](C)CCO1]

To a solution of sodium hydride (60% dispersion in mineral oil) (67 mg, 1.67 mmol) in dry DMF (100 mL) was added dropwise a solution of [(1R)-3-[(1S)-2-[6-[5-[tert-butyl(dimethyl)silyl]oxy-1-tetrahydropyran-2-yl-indazol-3-yl]pyrazin-2-yl]oxy-1-methyl-ethoxy]-1-methyl-propyl]methanesulfonate (354 mg, 0.558 mmol) in dry DMF (30 mL). The reaction mixture was stirred at RT for 16 h. Additional sodium hydride (60% dispersion in mineral oil) (22 mg, 0.558 mmol) was added and the reaction mixture was stirred at RT for 30 min. Additional sodium hydride (60% dispersion in mineral oil) (44 mg, 1.11 mmol) was added and the reaction mixture was stirred at RT for 1.5 h. The reaction mixture was quenched with water, concentrated under reduced pressure and diluted with water and ethyl acetate. The aqueous layer was extracted with ethyl acetate. The combined organic layers were washed with water and brine, dried over anhydrous sodium sulfate, filtered and evaporated under reduced pressure. The residue was purified by silica gel column chromatography using cyclohexane/ethyl acetate 100/0 to 70/30 as eluent. to afford (9S,13S)-9,13-dimethyl-19-(oxan-2-yl)-7,10,14-trioxa-4,19,20,23-tetraazatetracyclo[13.5.2.1^{2,6}.0^{18,21}]tricosa-1(20),2(23),3, 5, 15(22),16,18(21)-heptaene as a white solid.

LCMS method F: [M+H]⁺=425.3, t_{R}=3.20 min

### Preparation of Example 51: (9S,13S)-9,13-dimethyl-7,10,14-trioxa-4,19,20,23-tetraazatetracyclo[13.5.2.1^{2,6}.0^{18,21}]tricosa-1(20),2(23),3,5,15(22),16,18(21)-heptaene

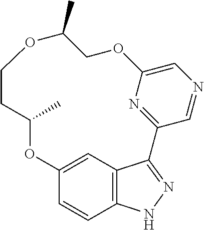 [SMILES: C[C@H]1COc2cncc(n2)-c2n[nH]c3ccc(cc23)O[C@@H](C)CCO1]

To a solution of (9S,13S)-9,13-dimethyl-19-(oxan-2-yl)-7,10,14-trioxa-4,19,20,23-tetraaza tetracyclo[13.5.2.1^{2,6}.0^{18,21}]tricosa-1(20),2(23),3,5,15(22),16,18(21)-heptaene (96 mg, 0.226 mmol) in methanol (17 mL) and water (2.4 mL) at RT was added p-toluenesulfonic acid monohydrate (215 mg, 1.13 mmol). The reaction mixture was stirred at 65° C. for 16 h. The reaction mixture was concentrated under reduced pressure and a saturated aqueous Na₂CO₃ solution and ethyl acetate were added. The aqueous layer was extracted with ethyl acetate. The combined organic layers were washed with water and brine, dried over anhydrous sodium sulfate, filtered and evaporated under reduced pressure. The resulting solid was recrystallized from acetonitrile to afford (9S,13S)-9,13-dimethyl-7,10,14-trioxa-4,19,20,23-tetraazatetra cyclo[13.5.2.1^{2,6}.0^{18,21}]tricosa-1(20),2(23),3,5,15(22),16,18(21)-heptaene as a solid.

LCMS method J: [M+H]⁺=341, t_{R}=3.56 min

LCMS method N: [M+H]⁺=341, t_{R}=3.54 min

¹H NMR (400 MHz, d6-DMSO) 13.35 (1H, s), 8.82 (1H, s), 8.58 (1H, m), 8.20 (1H, s), 7.50-7.47 (1H, d, J=9.1 Hz), 6.99-6.96 (1H, dd, J=2.5, 9.0 Hz), 5.53-5.50 (1H, d, J=12.4 Hz), 4.66-4.60 (1H, m), 4.16-4.12 (1H, dd, J=3.2, 12.1 Hz), 3.92-3.87 (2H, m), 3.24-3.17 (1H, m), 2.22-2.14 (1H, m), 1.48-1.40 (4H, m), 1.29 (3H, d, J=6.4 Hz) ppm.

### Example 52: (7R,12S)-7,12-dimethyl-9,13-dioxa-4,5,18,19-tetraazatetracyclo[12.5.2.1^{2,5}.0^{17,20}]docosa-1(19),2(22),3,14(21),15,17(20)-hexaene

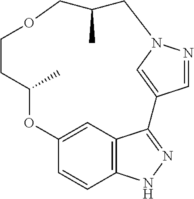 [SMILES: C[C@H]1COCC[C@H](C)Oc2ccc3[nH]nc(c3c2)-c2cnn(c2)C1]

Example 52 is prepared according to the synthesis route described in general Scheme D.

### Preparation of Intermediate 285: [(2S)-3-hydroxy-2-methyl-propyl] acetate

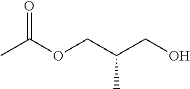

To a solution of 2-methylpropane-1,3-diol (6 mL, 67.9 mmol) in chloroform (44 mL) was added vinyl acetate (25 mL, 272 mmol) and amano lipase from pseudomonas fluorescen (1.1 g). The reaction mixture was stirred at RT for 28 h. The mixture was filtered and the filtrate was concentrated under reduced pressure. The residue was purified by silica gel column chromatography using cyclohexane/ethyl acetate 100/0 to 50/50 as eluent to afford [(2S)-3-hydroxy-2-methyl-propyl] acetate as a pale yellow liquid.

¹H NMR (400 MHz, CDCl₃): 4.08 (2H, m), 3.53 (2H, m), 2.25 (1H, m), 2.08 (3H, s), 1.99 (1H, m), 0.96 (3H, d, J=6.8 Hz) ppm.

### Preparation of Intermediate 286: (2R)-3-[(3R)-3-benzyloxybutoxy]-2-methyl-propan-1-ol

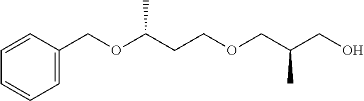 [SMILES: C[C@H](CO)COCC[C@@H](C)OCc1ccccc1]

To a solution of [(2S)-3-hydroxy-2-methyl-propyl] acetate (1.97 g, 14.9 mmol) in dry DMF (87 mL) at 0° C. was dropwise added sodium hydride (60% dispersion in mineral oil) (1.79 g, 44.7 mmol). The reaction mixture was stirred at 0° C. for 1 h. A solution of [(1R)-3-bromo-1-methyl-propoxy]methylbenzene (intermediate 166) (4.01 g, 16.5 mmol) in dry DMF (10 mL) was added dropwise and the reaction mixture was stirred at RT for 48 h. Water was added at 0° C. followed by 1 M aqueous HCl solution to adjust the pH to ˜ 7. The mixture was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford (2R)-3-[(3R)-3-benzyloxybutoxy]-2-methyl-propan-1-ol as a pale yellow liquid.

¹H NMR (400 MHz, CDCl₃): 7.37-7.27 (5H, m), 4.63-4.44 (2H, m), 3.84-3.45 (5H, m), 3.36 (1H, m), 2.77 (1H, m), 2.04 (1H, m), 1.89-1.72 (2H, m), 1.29-1.24 (3H, m), 0.96-0.86 (3H, m) ppm.

### Preparation of Intermediate 287: [(2S)-3-[(3R)-3-benzyloxybutoxy]-2-methyl-propyl] methanesulfonate

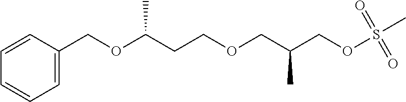 [SMILES: C[C@@H](COCC[C@@H](C)OCc1ccccc1)COS(C)(=O)=O]

To a solution of (2R)-3-[(3R)-3-benzyloxybutoxy]-2-methyl-propan-1-ol (1.2 g, 4.76 mmol) and triethylamine (0.99 mL, 7.14 mmol) in dichloromethane (20 mL) at 0° C. was added dropwise methanesulfonyl chloride (0.4 mL, 5.24 mmol). The reaction mixture was stirred at RT for 6 h then diluted with water and dichloromethane. The aqeous layer was extracted with dichloromethane. The combined organic layers were washed with a saturated ammonium chloride solution and brine, dried over anhydrous sodium sulfate, filtered and evaporated under reduced pressure to afford [(2S)-3-[(3R)-3-benzyloxybutoxy]-2-methyl-propyl] methane sulfonate as an orange oil which was used in the next step without further purification.

¹H NMR (400 MHz, CDCl₃): 7.35-7.30 (5H, m), 4.61-4.45 (2H, dd, J=61.8, 11.53 Hz), 4.18 (2H, m), 3.7 (1H, m), 3.54 (2H, m), 3.32 (2H, m), 2.98 (2H, s), 2.15 (1H, m), 1.84-1.74 (2H, m), 1.28-1.24 (3H, m), 1.00-0.95 (3H, m) ppm.

### Preparation of Intermediate 288: 1-[(2R)-3-[(3R)-3-benzyloxybutoxy]-2-methyl-propyl]-4-bromo-pyrazole

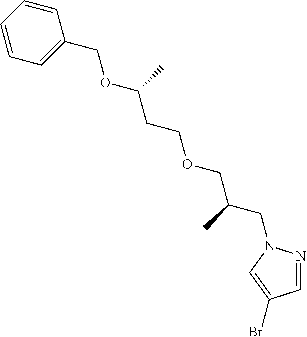 [SMILES: C[C@@H](COCC[C@@H](C)OCc1ccccc1)Cn1cc(Br)cn1]

To a solution of [(2S)-3-[(3R)-3-benzyloxybutoxy]-2-methyl-propyl] methanesulfonate (1.5 g, 4.55 mmol) in dry acetonitrile (30 mL) was added 4-bromo-1H-pyrazole (603 mg, 4.14 mmol) and cesium carbonate (2.02 g, 6.21 mmol) at RT. The reaction mixture was stirred at 85° C. overnight. The reaction mixture diluted with water and ethyl acetate. The aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford 1-[(2R)-3-[(3R)-3-benzyloxybutoxy]-2-methyl-propyl]-4-bromo-pyrazole as a colorless oil.

LCMS method F: [M+H]⁺=381.1-383.1, t_{R}=3.14 min

### Preparation of Intermediate 289: [3-[1-[(2R)-3-[(3R)-3-benzyloxybutoxy]-2-methyl-propyl]pyrazol-4-yl]-1-tetrahydropyran-2-yl-indazol-5-yl]oxy-tert-butyl-dimethyl-silane

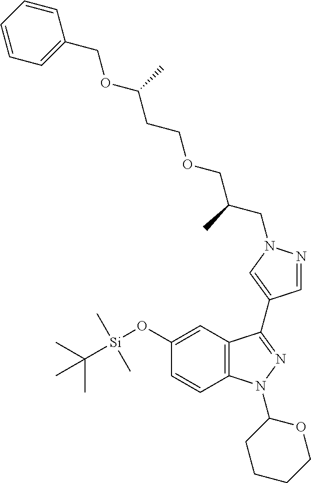 [SMILES: C[C@@H](COCC[C@@H](C)OCc1ccccc1)Cn1cc(-c2nn(C3CCCCO3)c3ccc(O[Si](C)(C)C(C)(C)C)cc23)cn1]

To a degassed suspension of 1-[(2R)-3-[(3R)-3-benzyloxybutoxy]-2-methyl-propyl]-4-bromo-pyrazole (1.35 g, 3.55 mmol) tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (2.28 g, 4.97 mmol), potassium phosphate tribasic (2.26 g, 10.65 mmol) and XPhos (169 mg, 0.36 mmol) in dioxane (40 mL) and water (4.4 mL) was added tetrakis(triphenylphosphine)palladium(0) (205 mg, 0.18 mmol). The reaction mixture was stirred at 90° C. overnight The reaction mixture was filtered through a pad of Celite and washed with ethyl acetate. Water was added to the filtrate and the aqueous layer was extracted with ethyl acetate. The combined organic layer was washed with brine, dried over anhydrous sodium sulfate, filtered and evaporated under reduced pressure. The residue was purified by silica gel column chromatography using cyclohexane/ethyl acetate 100/0 to 60/40 as eluent to afford [3-[1-[(2R)-3-[(3R)-3-benzyloxy butoxy]-2-methyl-propyl]pyrazol-4-yl]-1-tetrahydropyran-2-yl-indazol-5-yl]oxy-tert-butyl-dimethyl-silane as an orange/red oil.

LCMS method F: [M+H]⁺=633.4, t_{R}=3.94 min

### Preparation of Intermediate 290: (2R)-4-[(2R)-3-[4-[5-[tert-butyl(dimethyl)silyl]oxy-1-tetrahydropyran-2-yl-indazol-3-yl]pyrazol-1-yl]-2-methyl-propoxy]butan-2-ol

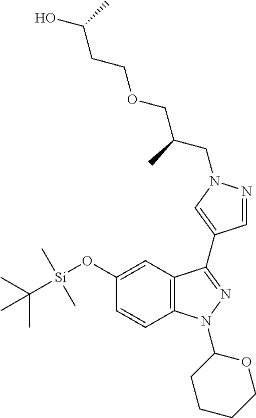 [SMILES: C[C@@H](COCC[C@@H](C)O)Cn1cc(-c2nn(C3CCCCO3)c3ccc(O[Si](C)(C)C(C)(C)C)cc23)cn1]

To a solution of [3-[1-[(2R)-3-[(3R)-3-benzyloxybutoxy]-2-methyl-propyl]pyrazol-4-yl]-1-tetrahydropyran-2-yl-indazol-5-yl]oxy-tert-butyl-dimethyl-silane (1.06 g, 1.67 mmol) in ethyl acetate (25 mL) at RT was added Pd(OH)₂/C (106 mg). The reaction mixture was heated under hydrogen atmosphere at 60° C. for 24 h. The reaction mixture was filtered and washed with ethyl acetate. The solvent was removed under reduced pressure to afford (2R)-4-[(2R)-3-[4-[5-[tert-butyl(dimethyl)silyl]oxy-1-tetrahydropyran-2-yl-indazol-3-yl]pyrazol-1-yl]-2-methyl-propoxy]butan-2-ol as a clear brown oil which was used in the next step without further purification.

LCMS method F: [M+H]⁺=543.3, t_{R}=3.47 min

### Preparation of Intermediate 291: [(1R)-3-[(2R)-3-[4-[5-[tert-butyl(dimethyl)silyl] oxy-1-tetrahydropyran-2-yl-indazol-3-yl]pyrazol-1-yl]-2-methyl-propoxy]-1-methyl-propyl]methanesulfonate

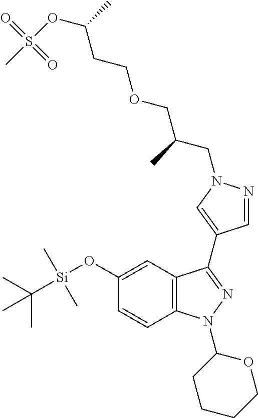 [SMILES: C[C@@H](COCC[C@@H](C)OS(C)(=O)=O)Cn1cc(-c2nn(C3CCCCO3)c3ccc(O[Si](C)(C)C(C)(C)C)cc23)cn1]

To a solution of (2R)-4-[(2R)-3-[4-[5-[tert-butyl(dimethyl)silyl]oxy-1-tetrahydropyran-2-yl-indazol-3-yl]pyrazol-1-yl]-2-methyl-propoxy]butan-2-ol (853 mg, 1.5 mmol) and triethylamine (420 μL, 3 mmol) in dichloromethane (20 mL) at 0° C., was added dropwise methanesulfonyl chloride (150 □L, 1.95 mmol). The reaction mixture was stirred at RT for 3 h. The reaction mixture was diluted with water and extracted with dichloromethane. The combined organic layers were washed with brine, dried over anhydrous sodium sulfate, filtered and evaporated under reduced pressure to afford [(1R)-3-[(2R)-3-[4-[5-[tert-butyl(dimethyl)silyl]oxy-1-tetrahydropyran-2-yl-indazol-3-yl]pyrazol-1-yl]-2-methyl-propoxy]-1-methyl-propyl] methanesulfonate as a pale brown oil which was used in the next step without further purification.

LCMS method F: [M+H]⁺=621.3, t_{R}=3.55 min

### Preparation of Intermediate 292: (7R,12S)-7,12-dimethyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene

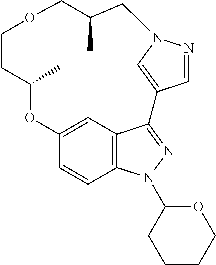 [SMILES: C[C@H]1COCC[C@H](C)Oc2ccc3c(c2)c(nn3C2CCCCO2)-c2cnn(c2)C1]

To a suspension of cesium carbonate (1.46 g, 4.5 mmol) in anhydrous DMF (500 mL) at 80° C. was added dropwise [(1R)-3-[(2R)-3-[4-[5-[tert-butyl(dimethyl)silyl]oxy-1-tetrahydropyran-2-yl-indazol-3-yl]pyrazol-1-yl]-2-methyl-propoxy]-1-methyl-propyl] methanesulfonate (1.04 g, 1.5 mmol) in DMF (500 mL). After addition the resulting reaction mixture was stirred at 80° C. for 3 h. The reaction mixture was allowed to cool down to RT, filtered and washed with ethyl acetate. The filtrate was concentrated under reduced pressure. The residue was purified by silica gel column chromatography using dichloromethane/methanol 100/0 to 95/5 as eluent. The resulting oil was purified by preparative TLC with dichloromethane/methanol 95/5 as eluent to afford (7R,12S)-7,12-dimethyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene as an orange solid.

LCMS method F: [M+H]⁺=411.5, t_{R}=2.87 min

### Preparation of Example 52: (7R,12S)-7,12-dimethyl-9,13-dioxa-4,5,18,19-tetraaza tetracyclo[12.5.2.1^{2,5}.0^{17,20}]docosa-1(19),2(22),3,14(21),15,17(20)-hexaene

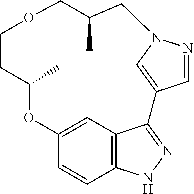 [SMILES: C[C@H]1COCC[C@H](C)Oc2ccc3[nH]nc(c3c2)-c2cnn(c2)C1]

To a solution of (7R,12S)-7,12-dimethyl-18-(oxan-2-yl)-9,13-dioxa-4,5,18,19-tetraazatetra cyclo[12.5.2.1^{2,5}.0^{17,20}]docosa-1(19),2(22),3,14(21),15,17(20)-hexaene (37 mg, 0.09 mmol) in methanol (1.5 mL) and water (0.25 mL) was added p-toluenesulfonic acid monohydrate (86 mg, 0.45 mmol) and the reaction mixture was stirred at 65° C. overnight. The reaction mixture was diluted with dichloromethane and saturated aqueous NaHCO₃ solution. The aqueous layer was extracted with dichloromethane. The combined organic layers were washed with brine, dried over anhydrous sodium sulfate, filtered and evaporated under reduced pressure. The residue was purified by preparative TLC using dichloromethane/methanol 90/10 as eluent. The resulting solid was triturated with diisopropylether, filtered and dried to afford (7R,12S)-7,12-dimethyl-9,13-dioxa-4,5,18,19-tetraazatetracyclo[12.5.2.1^{2,5}.0^{17,20}]docosa-1(19),2(22),3, 14(21),15,17(20)-hexaene as a cream solid.

LCMS method F: [M+H]+=327.3, tR=2.30 min

LCMS method G: [M+H]+=327.3, tR=2.29 min 1H NMR (400 MHz, MeOD) 8.57 (1H, dd, J=0.6, 12.3 Hz), 7.74 (1H, m), 7.49 (1H, dd, J=2.5, 4.2 Hz), 7.40 (1H, dd, J=3.6, 8.9 Hz), 7.01 (1H, td, J=2.4, 9.0 Hz), 4.5 (2H, m), 4.32 (1H, m), 3.83-3.49 (4H, m), 2.63 (1H, m), 2.29 (1H, m), 1.54 (1H, m), 1.46 (3H, dd, J=1.5, 6 Hz), 1.13 (2H, t, J=6.8 Hz), 0.93 (1H, d, J=7.2 Hz) ppm.

### Example 53: (8R,13S)-8,13-dimethyl-7,11,14-trioxa-5,19,20,23-tetraazatetracyclo[13.5.2.1^{2,6}.0^{18,21}]tricosa-1(20),2(23),3,5,15(22),16,18(21)-heptaene

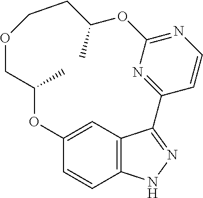 [SMILES: C[C@@H]1CCOC[C@H](C)Oc2ccc3[nH]nc(c3c2)-c2ccnc(n2)O1]

Example 53 is prepared according to the synthesis route described in general Scheme C.

### Preparation of Intermediate 293: 2-[(1R)-3-[(2R)-2-benzyloxy propoxy]-1-methyl-propoxy]tetrahydropyran

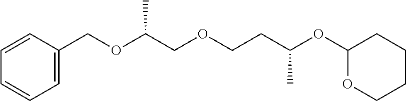 [SMILES: C[C@H](COCC[C@@H](C)OC1CCCCO1)OCc1ccccc1]

To a solution of (3R)-3-tetrahydropyran-2-yloxybutan-1-ol (intermediate 236) (1.9 g, 10.9 mmol) in DMF (45 mL) at 0° C. was added portionwise sodium hydride (60% dispersion in mineral oil) (523 mg, 13.08 mmol). The reaction mixture was stirred at 0° C. for 5 min and a solution of [(2R)-2-benzyloxypropyl]-4-methylbenzenesulfonate (intermediate 88) (4.2 g, 12 mmol) in DMF (15 mL) was added dropwise. The reaction mixture was stirred at RT for 10 min and at 70° C. overnight. Additional sodium (60% dispersion in mineral oil) (262 mg, 5.45 mmol) was added at RT and the reaction mixture was stirred at 70° C. for 5 h. Water was added dropwise and the aqueous layer was extracted with ethyl acetate. The combined organic layers were washed with brine, dried over anhydrous magnesium sulfate, filtered and evaporated under reduced pressure. The residue was purified by silica gel column chromatography using cyclohexane/ethyl acetate 100/0 to 70/30 as eluent to afford 2-[(1R)-3-[(2R)-2-benzyloxy propoxy]-1-methyl-propoxy]tetrahydropyran as a colorless oil.

LCMS method F: [M+Na]⁺=345.2, t_{R}=3.07 min

### Preparation of Intermediate 294: (2R)-4-[(2R)-2-benzyloxypropoxy]butan-2-ol

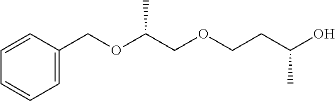 [SMILES: C[C@H](COCC[C@@H](C)O)OCc1ccccc1]

To a solution of 2-[(1R)-3-[(2R)-2-benzyloxypropoxy]-1-methyl-propoxy]tetrahydropyran (1.04 g, 3.23 mmol) in methanol (27 mL) and water (7 mL) was added p-toluenesulfonic acid monohydrate (1.84 g, 9.69 mmol). The reaction mixture was stirred at 65° C. overnight. The solvent was evaporated under reduced pressure. The residue was diluted with a saturated NaHCO₃ aqueous solution and ethyl acetate. The aqueous layer was extracted with ethyl acetate. The combined organic layers were washed with brine, dried over anhydrous magnesium sulfate, filtered and evaporated under reduced pressure to afford (2R)-4-[(2R)-2-benzyloxy propoxy]butan-2-ol as a colorless oil which was used in the next step without further purification.

¹H NMR (400 MHz, DMSO) 7.35-7.31 (4H, m), 7.29-7.24 (1H, m), 4.54 (2H, d, J=2.4 Hz), 4.37 (2H, d, J=4.8 Hz) 3.75-3.67 (1H, m), 3.67-3.60 (1H, m), 3.52-3.39 (3H, m), 3.32 (1H, dd, J=4.6. 10.1 Hz), 1.56 (1H, q, J=6.6 Hz), 1.10 (3H, t, J=6.3 Hz), 1.06 (3H, t, J=6.1 Hz) ppm.

### Preparation of Intermediate 295: [3-[2-[(1R)-3-[(2R)-2-benzyloxypropoxy]-1-methyl-propoxy]pyrimidin-4-yl]-1-tetrahydropyran-2-yl-indazol-5-yl]oxy-tert-butyl-dimethyl-silane

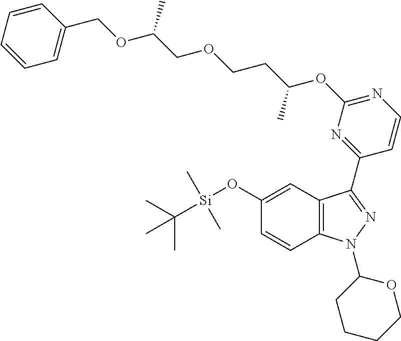 [SMILES: C[C@H](COCC[C@@H](C)Oc1nccc(-c2nn(C3CCCCO3)c3ccc(O[Si](C)(C)C(C)(C)C)cc23)n1)OCc1ccccc1]

To a solution of (2R)-4-[(2R)-2-benzyloxypropoxy]butan-2-ol (390 mg, 1.64 mmol) in THF (10 mL) at 0° C. was added sodium hydride (60% dispersion in mineral oil) (67 mg, 1.76 mmol) and the reaction mixture was stirred at RT for 10 min. The reaction mixture was cooled to 0° C. and a solution of tert-butyl-dimethyl-[3-(2-methylsulfonylpyrimidin-4-yl)-1-tetrahydropyran-2-yl-indazol-5-yl]oxy-silane (intermediate 63) (800 mg, 1.64 mmol) in THF (5 mL) was added dropwise. The reaction mixture was stirred at 0° C. for 5 min and RT for 1.5 h. A saturated aqueous ammonium chloride solution was added and the mixture was poured in ethyl acetate. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 70/30 as eluent to afford [3-[2-[(1R)-3-[(2R)-2-benzyloxypropoxy]-1-methyl-propoxy]pyrimidin-4-yl]-1-tetrahydropyran-2-yl-indazol-5-yl]oxy-tert-butyl-dimethyl-silane as a colorless oil.

¹H NMR (400 MHz, DMSO) 8.60 (1H, dd, J=0.4, 5.2 Hz), 7.95 (1H, d, J=2.4 Hz), 7.64 (1H, d, J=9.2 Hz), 7.69 (1H, dd, J=0.8, 5.2 Hz), 7.34-7.29 (1H, m), 7.25-7.18 (4H, m), 7.10 (1H, dd, J=2.3, 9.1 Hz), 5.98-5.94 (1H, m), 5.47-5.40 (1H, m), 4.42 (2H, d, J=2.0 Hz), 3.95-3.87 (1H, m), 3.82-3.74 (1H, m), 3.60-3.52 (3H, m), 3.45-3.40 (1H, m), 3.2-3.22 (1H, m), 2.49-2.39 (1H, m), 2.08-1.90 (4H, m), 1.83-1.72 (1H, m), 1.66-1.57 (2H, m), 1.41-1.38 (6H, m), 0.99-0.98 (9H, m), 0.21-0.20 (6H, m) ppm.

### Preparation of Intermediate 296: (2R)-1-[(3R)-3-[4-[5-[tert-butyl(dimethyl)silyl]oxy-1-tetrahydropyran-2-yl-indazol-3-yl]pyrimidin-2-yl]oxybutoxy]propan-2-ol

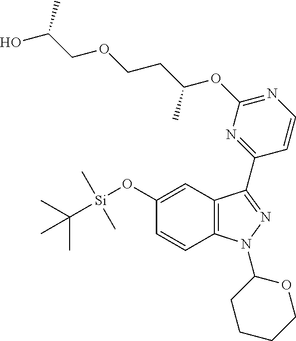 [SMILES: C[C@H](CCOC[C@@H](C)O)Oc1nccc(-c2nn(C3CCCCO3)c3ccc(O[Si](C)(C)C(C)(C)C)cc23)n1]

To a solution of [3-[2-[(1R)-3-[(2R)-2-benzyloxypropoxy]-1-methyl-propoxy]pyrimidin-4-yl]-1-tetrahydropyran-2-yl-indazol-5-yl]oxy-tert-butyl-dimethyl-silane (385 mg, 0.6 mmol) in ethanol (5 mL) under nitrogen atmosphere was added palladium hydroxide (126 mg, 0.9 mmol). The reaction mixture was stirred at 60° C. for 72 h under hydrogen atmosphere. The reaction mixture was filtered and the filtrate was concentrated under reduced pressure. The residue was purified by silica gel column chromatography using cyclohexane/ethyl acetate 50/50 as eluent to afford (2R)-1-[(3R)-3-[4-[5-[tert-butyl(dimethyl)silyl]oxy-1-tetrahydro pyran-2-yl-indazol-3-yl]pyrimidin-2-yl]oxybutoxy]propan-2-ol as a colorless oil.

LCMS method F: [M+H]⁺=557.5, t_{R}=3.63 min

### Preparation of Intermediate 297: 3-[2-[(1R)-3-[(2R)-2-hydroxypropoxy]-1-methyl-propoxy]pyrimidin-4-yl]-1-tetrahydropyran-2-yl-indazol-5-ol

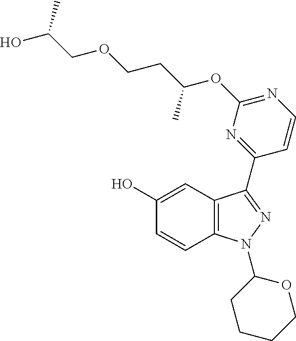 [SMILES: C[C@H](CCOC[C@@H](C)O)Oc1nccc(-c2nn(C3CCCCO3)c3ccc(O)cc23)n1]

To stirred solution of (2R)-1-[(3R)-3-[4-[5-[tert-butyl(dimethyl)silyl]oxy-1-tetrahydropyran-2-yl-indazol-3-yl]pyrimidin-2-yl]oxybutoxy]propan-2-ol (170 mg, 0.31 mmol) in THF (3 mL) at 0° C. was added TBAF (1M solution in THF) (310 μL, 0.31 mmol). The reaction mixture was stirred at RT for 2 h. Ice water was added and the mixture was stirred for 15 min. ethyl acetate (20 mL) was added and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 0/100 as eluent to afford 3-[2-[(1R)-3-[(2R)-2-hydroxypropoxy]-1-methyl-propoxy]pyrimidin-4-yl]-1-tetrahydropyran-2-yl-indazol-5-ol as a colorless oil.

LCMS method F: [M+H]⁺=443.3, t_{R}=2.41 min

### Preparation of Intermediate 298: (8R,13S)-8,13-dimethyl-19-(oxan-2-yl)-7,11,14-trioxa-5,19,20,23-tetraazatetracyclo[13.5.2.1^{2,6}.0^{18,21}]tricosa-1(20),2(23),3,5,15(22), 16,18(21)-heptaene

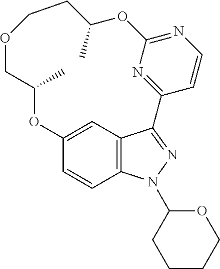 [SMILES: C[C@@H]1CCOC[C@H](C)Oc2ccc3c(c2)c(nn3C2CCCCO2)-c2ccnc(n2)O1]

To a solution of diisopropyl azodicarboxylate (204 μL, 1.04 mmol) in THF (11 mL) at 60° C. was added a solution of triphenylphosphine (272 mg, 1.04 mmol) in toluene (12 mL) and a solution of 3-[2-[(1R)-3-[(2R)-2-hydroxypropoxy]-1-methyl-propoxy]pyrimidin-4-yl]-1-tetra hydropyran-2-yl-indazol-5-ol (153 mg, 0.35 mmol) in THF (12 mL). The reaction mixture was stirred at 60° C. for 3 h. The solvent was evaporated under reduced pressure. The residue was partitioned between ethyl acetate and water. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent to afford (8R,13S)-8,13-dimethyl-19-(oxan-2-yl)-7,11,14-trioxa-5,19,20,23-tetraazatetracyclo[13.5.2.1^{2,6}.0^{18,21}] tricosa-1(20),2(23),3,5,15(22),16,18(21)-heptaene as a yellow oil.

LCMS method F: [M+H]⁺=425.4, t_{R}=3.16 min

### Preparation of Example 53: (8R,13S)-8,13-dimethyl-7,11,14-trioxa-5,19,20,23-tetraazatetracyclo[13.5.2.1^{2,6}.0^{18,21}]tricosa-1(20),2(23),3,5,15(22),16,18(21)-heptaene

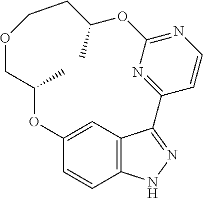 [SMILES: C[C@@H]1CCOC[C@H](C)Oc2ccc3[nH]nc(c3c2)-c2ccnc(n2)O1]

To a solution of (8R,13S)-8,13-dimethyl-19-(oxan-2-yl)-7,11,14-trioxa-5,19,20,23-tetraaza tetracyclo[13.5.2.1^{2,6}.0^{18,21}]tricosa-1(20),2(23),3,5,15(22),16,18(21)-heptaene (187 mg, 0.44 mmol) in methanol (4 mL) and water (0.7 mL) was added p-toluenesulfonic acid monohydrate (419 mg, 2.2 mmol). The reaction mixture was stirred at 80° C. for 18 h. The solvent was removed under reduced pressure. The residue was partitioned between dichloromethane and a saturated aqueous NaHCO₃ solution. The aqueous layer was extracted with dichloromethane. The organic layers were washed with brine, dried over anhydrous sodium sulfate, filtered and evaporated under reduced pressure. The residue was purified by silica gel column chromatography using cyclohexane/ethyl acetate 50/50 as eluent. The resulting solid was triturated with diisopropylether, filtered and dried to afford (8R,13S)-8,13-dimethyl-7,11,14-trioxa-5,19,20,23-tetraazatetracyclo[13.5.2.1^{2,6}.0^{18,21}]tricosa-1(20),2(23),3,5,15(22),16, 18(21)-heptaene as a solid.

LCMS method F: [M+H]+=341.3, tR=2.42 min

LCMS method G: [M+H]+=341.3, tR=2.43 min 1H NMR (400 MHz, d6-DMSO) 13.60 (1H, s), 8.87 (1H, d, J=2.1 Hz), 8.57 (1H, d, J=5.1 Hz), 7.75 (1H, d, J=5.1 Hz), 7.50-7.47 (1H, m), 7.04 (1H, dd, J=2.3, 8.9 Hz), 5.72-5.65 (1H, m), 4.44-4.38 (1H, m), 3.73 (1H, dd, J=7.3, 10.0 Hz), 3.65-3.51 (3H, m), 2.5 (1H, m, under Me of DMSO confirmed by COSY) 1.47-1.44 (4H, m), 1.34-1.31 (3H, m) ppm.

### Example 54: (12S)-12-methyl-8,11,14-trioxa-4,5,19,20-tetraazatetracyclo[13.5.2.1^{2,5}.0^{18,21}]tricosa-1(20),2(23),3,15(22),16,18(21)-hexaene

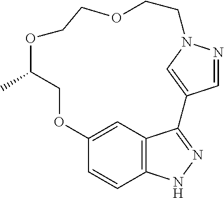 [SMILES: C[C@H]1COc2ccc3[nH]nc(c3c2)-c2cnn(c2)CCOCCO1]

Example 54 is prepared according to the synthesis route described in general Scheme D.

### Preparation of Intermediate 299: 2-(((S)-1-(benzyloxy)propan-2-yl)oxy)tetrahydro-2H-pyran

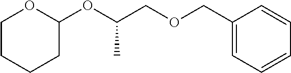 [SMILES: C[C@@H](COCc1ccccc1)OC1CCCCO1]

(2S)-2-tetrahydropyran-2-yloxypropan-1-ol was prepared according to the same procedure as for (2R)-2-tetrahydropyran-2-yloxypropan-1-ol (intermediate 131), starting from methyl (2S)-2-hydroxypropanoate.

To a solution of (2S)-2-tetrahydropyran-2-yloxypropan-1-ol (24.1 g, 150.42 mmol) in dry DMF (225 mL) at 0° C. was added dropwise sodium hydride (60% dispersion in mineral oil) (9.02 g, 225.64 mmol) and the reaction mixture was stirred at 0° C. for 1 h. A solution of benzyl bromide (35.73 mL, 300.85 mmol) was added dropwise and the reaction mixture was stirred at RT for 3 h. The reaction mixture was quenched by water and the aqueous layer was extracted with ethyl acetate. The combined organic layers were washed with brine, dried over anhydrous magnesium sulfate, filtered and evaporated under reduced pressure. The residue was purified by silica gel column chromatography using heptane/ethyl acetate 100/0 to 80/20 as eluent to afford 2-(((S)-1-(benzyloxy)propan-2-yl)oxy)tetrahydro-2H-pyran as a colorless oil.

GCMS method A [M−H]⁻=249.1, t_{R}=9.595 min

### Preparation of Intermediate 300: (2S)-1-(benzyloxy)propan-2-ol

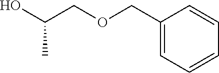

To a solution of 2-(((S)-1-(benzyloxy)propan-2-yl)oxy)tetrahydro-2H-pyran (35.65 g, 142.40 mmol) in methanol (182 mL) at 0° C. was added p-toluenesulfonic acid monohydrate (1.35 g, 7.12 mmol) and the reaction mixture was stirred at RT for 18 h. The reaction mixture was cooled at 0° C. and a saturated aqueous NaHCO₃ solution was added. The solvent was evaporated under reduced pressure. The residue was partitioned between diethyl ether and water. The phases were separated and the organic layer was dried over anhydrous magnesium sulfate, filtered and evaporated under reduced pressure. The residue was purified by silica gel column chromatography using heptane/ethyl acetate 100/0 to 75/25 as eluent to afford (S)-1-(benzyloxy)propan-2-ol as a colorless oil.

GCMS method A [M+H]⁺=166.1, t_{R}=6.709 min

### Preparation of Intermediate 301: 2-(2-(4-bromo-1H-pyrazol-1-yl)ethoxy)ethyl-4-methylbenzenesulfonate

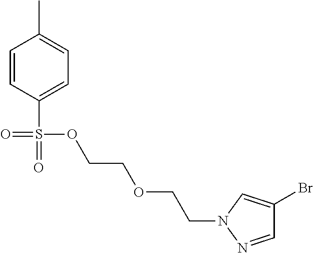 [SMILES: Cc1ccc(S(=O)(=O)OCCOCCn2cc(Br)cn2)cc1]

To a solution of sodium hydroxide (493 mg, 12.31 mmol) and 2-[2-(4-bromopyrazol-1-yl)ethoxy]ethanol (intermediate 147) (1.93 g, 8.21 mmol) in water (2.5 mL) and THF (19.5 mL) at 0° C. was added dropwise p-toluenesulfonyl chloride (1.878 g, 9.852 mmol) in THF (2.5 mL). The reaction mixture was stirred at 0° C. for 3 h. The reaction mixture was poured into ice-water and extracted with dichloromethane. The combined organic layers were dried over anhydrous magnesium sulfate, filtered and evaporated under reduced pressure. The residue was purified by silica gel column chromatography using heptane/ethyl acetate 100/0 to 50/50 as eluent to afford 2-(2-(4-bromo-1H-pyrazol-1-yl)ethoxy)ethyl 4-methylbenzenesulfonate as a colorless oil.

LCMS method B: [M+H]⁺=388.8-390.9, t_{R}=0.892 min

### Preparation of Intermediate 302: 1-[2-[2-[(1S)-2-benzyloxy-1-methyl-ethoxy]ethoxy] ethyl]-4-bromo-pyrazole

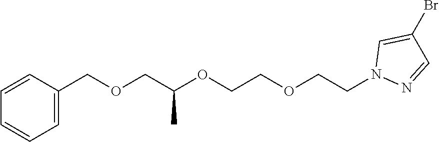 [SMILES: C[C@@H](COCc1ccccc1)OCCOCCn1cc(Br)cn1]

A mixture of (S)-1-(benzyloxy)propan-2-ol (990 mg, 5.956 mmol), 2-(2-(4-bromo-1H-pyrazol-1-yl)ethoxy)ethyl 4-methylbenzenesulfonate (2.66 g, 6.84 mmol) and potassium hydroxide (1.12 g, 20.01 mmol) in THF (18 mL) was stirred at 80° C. for 16 h. The reaction mixture was diluted with dichloromethane, water was added and the aqueous layer was extracted with dichloromethane. The combined organic layers were dried over anhydrous magnesium sulfate, filtered and evaporated under reduced pressure. The residue was purified by silica gel column chromatography using heptane/ethyl acetate 100/0 to 85/15 as eluent to afford 1-[2-[2-[(1S)-2-benzyloxy-1-methyl-ethoxy]ethoxy]ethyl]-4-bromo-pyrazole as a colorless oil.

LCMS method E: [M+H]⁺=382.9-384.9, t_{R}=3.733 min

### Preparation of Intermediate 303: [3-[1-[2-[2-[(1S)-2-benzyloxy-1-methyl-ethoxy]ethoxy]ethyl]pyrazol-4-yl]-1-tetrahydropyran-2-yl-indazol-5-yl]oxy-tert-butyl-dimethyl-silane

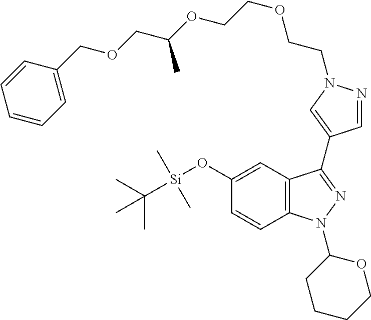 [SMILES: C[C@@H](COCc1ccccc1)OCCOCCn1cc(-c2nn(C3CCCCO3)c3ccc(O[Si](C)(C)C(C)(C)C)cc23)cn1]

To a degassed solution of 1-[2-[2-[(1S)-2-benzyloxy-1-methyl-ethoxy]ethoxy]ethyl]-4-bromo-pyrazole (940 mg, 2.45 mmol), tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (1.346 g, 2.94 mmol) and potassium phosphate tribasic (1.558 g, 7.35 mmol) in dioxane (35 mL) and water (2 mL) was added tetrakis(triphenylphosphine)palladium(0) (138 mg, 0.12 mmol) and 2-dicyclohexylphosphino-2′,4′,6′-triisopropylbiphenyl (114 mg, 0.24 mmol). The reaction mixture was stirred at 140° C. for 3 h. The reaction mixture was filtered over a pad Celite and washed with ethyl acetate. The filtrate was diluted with water and extracted with ethyl acetate. The combined organic layers were washed with water and brine, dried over anhydrous magnesium sulfate, filtered and evaporated under reduced pressure. The residue was purified by silica gel column chromatography using cyclohexane/ethyl acetate 99/1 to 40/60 as eluent to afford [3-[1-[2-[2-[(1S)-2-benzyloxy-1-methyl-ethoxy]ethoxy]ethyl]pyrazol-4-yl]-1-tetra hydropyran-2-yl-indazol-5-yl]oxy-tert-butyl-dimethyl-silane as a pink oil.

LCMS method F: [M+H]⁺=635.4, t_{R}=3.71 min

### Preparation of Intermediate 304: (2S)-2-[2-[2-[4-[5-[tert-butyl(dimethyl)silyl]oxy-1-tetrahydropyran-2-yl-indazol-3-yl]pyrazol-1-yl]ethoxy]ethoxy]propan-1-ol

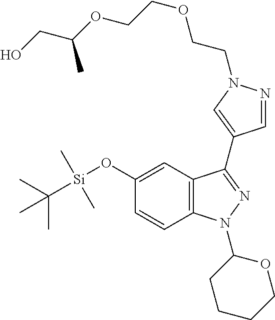 [SMILES: C[C@@H](CO)OCCOCCn1cc(-c2nn(C3CCCCO3)c3ccc(O[Si](C)(C)C(C)(C)C)cc23)cn1]

To a solution of [3-[1-[2-[2-[(1S)-2-benzyloxy-1-methyl-ethoxy]ethoxy]ethyl]pyrazol-4-yl]-1-tetrahydropyran-2-yl-indazol-5-yl]oxy-tert-butyl-dimethyl-silane (1.308 g, 2.06 mmol) in ethanol (30 mL) at RT was added palladium hydroxide on carbon (150 mg). The reaction mixture was stirred under hydrogen atmosphere at RT overnight. The reaction mixture filtered and the filtrate was concentrated under reduced pressure. The residue was purified by silica column chromatography using cyclohexane/ethyl acetate 100/0 to 20/80 as eluent to afford (2S)-2-[2-[2-[4-[5-[tert-butyl(dimethyl)silyl]oxy-1-tetrahydropyran-2-yl-indazol-3-yl] pyrazol-1-yl]ethoxy]ethoxy]propan-1-ol as a colorless oil.

LCMS method F: [M+H]⁺=545.4, t_{R}=3.19 min

### Preparation of Intermediate 305: [(2S)-2-[2-[2-[4-[5-[tert-butyl(dimethyl)silyl]oxy-1-tetrahydropyran-2-yl-indazol-3-yl]pyrazol-1-yl]ethoxy]ethoxy]propyl]methane sulfonate

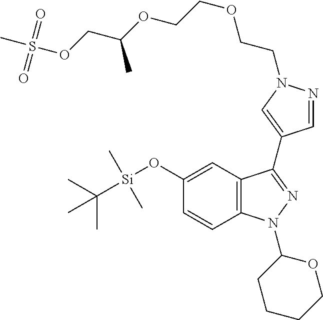 [SMILES: C[C@@H](COS(C)(=O)=O)OCCOCCn1cc(-c2nn(C3CCCCO3)c3ccc(O[Si](C)(C)C(C)(C)C)cc23)cn1]

To a solution of (2S)-2-[2-[2-[4-[5-[tert-butyl(dimethyl)silyl]oxy-1-tetrahydropyran-2-yl-indazol-3-yl]pyrazol-1-yl]ethoxy]ethoxy]propan-1-ol (815 mg, 1.5 mmol) and triethylamine (420 μL, 3 mmol) in dichloromethane (18 mL) at 0° C. was added dropwise methanesulfonyl chloride (150 μL, 1.95 mmol). The reaction mixture was stirred at RT for 2 h. The reaction mixture was diluted with water and extracted with dichloromethane. The combined organic layers were washed with brine, dried over anhydrous magnesium sulfate, filtered and evaporated under reduced pressure to afford [(2S)-2-[2-[2-[4-[5-[tert-butyl(dimethyl)silyl]oxy-1-tetrahydropyran-2-yl-indazol-3-yl]pyrazol-1-yl]ethoxy]ethoxy]propyl]methanesulfonate as a colorless oil which was used in the next step without further purification.

LCMS method F: [M+H]⁺=623.5, t_{R}=3.34 min

### Preparation of Intermediate 306: (12S)-12-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene

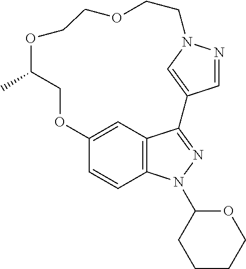 [SMILES: C[C@H]1COc2ccc3c(c2)c(nn3C2CCCCO2)-c2cnn(c2)CCOCCO1]

To a suspension of cesium carbonate (1.384 g, 4.26 mmol) in anhydrous DMF (80 mL) at 70° C. was added dropwise [(2S)-2-[2-[2-[4-[5-[tert-butyl(dimethyl)silyl]oxy-1-tetrahydropyran-2-yl-indazol-3-yl]pyrazol-1-yl]ethoxy]ethoxy]propyl] methanesulfonate (885 mg, 1.42 mmol) in DMF (30 mL). The reaction mixture was stirred at 70° C. for 16 h. The reaction mixture was allowed to cool down to RT, filtered over a pad of Celite and washed with ethyl acetate. The filtrate was concentrated under reduced pressure. The residue was purified by silica gel column chromatography using cyclohexane/ethyl acetate 100/0 to 30/70 as eluent to afford (12S)-12-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene as a colorless oil.

LCMS method F: [M+H]⁺=413.3, t_{R}=2.51 min

### Preparation of Example 54: (12S)-12-methyl-8,11,14-trioxa-4,5,19,20-tetraazatetra cyclo[13.5.2.1^{2,5}.0^{18,21}]tricosa-1(20),2(23),3,15(22),16,18(21)-hexaene

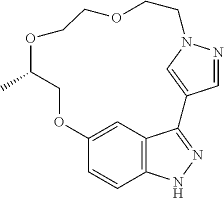 [SMILES: C[C@H]1COc2ccc3[nH]nc(c3c2)-c2cnn(c2)CCOCCO1]

To a solution of (12S)-12-methyl-19-(oxan-2-yl)-8,11,14-trioxa-4,5,19,20-tetraazatetracyclo [13.5.2.1^{2,5}.0^{18,21}]tricosa-1(20),2(23),3,15(22),16,18(21)-hexaene (511 mg, 1.24 mmol) in dichloromethane (7.5 mL) at RT was added TFA (1.85 mL, 24.8 mmol). The reaction mixture was stirred at RT for 48 h. The reaction mixture was concentrated under reduced pressure, diluted with saturated NaHCO₃ solution and extracted with ethyl acetate. The combined organic layers were dried over anhydrous sodium sulfate, filtered and evaporated under reduced pressure. The residue was purified by silica gel column chromatography using dichloromethane/methanol 100/0 to 97/3 as eluent to afford pure (12S)-12-methyl-8,11,14-trioxa-4,5,19,20-tetraazatetracyclo[13.5.2.1^{2,5}.0^{18,21}]tricosa-1(20),2(23),3,15(22),16,18(21)-hexaene as foam.

LCMS method F: [M+H]+=329.2, t_{R}=2.01 min

LCMS method G: [M+H]+=329.2, t_{R}=1.91 min 1H NMR (400 MHz, CDCl₃) 8.55 (1H, d, J=0.8 Hz), 8.01 (1H, d, J=0.8 Hz), 7.95 (1H, d, J=2.0 Hz), 7.35 (1H, dd, J=8.0, 0.8 Hz), 7.11 (1H, dd, J=2.3, 8.9 Hz), 4.50-4.47 (2H, m), 4.37-4.33 (2H, m), 3.92-3.81 (4H, m), 3.79-3.66 (2H, m), 3.65-3.57 (1H, m), 1.35 (3H, d, J=6.4 Hz) ppm.

### Example 55: (8R)-8-methyl-7,10,14-trioxa-5,19,20,23-tetraazatetracyclo[13.5.2.1^{2,6}.0^{18,21}] tricosa-1(20),2(23),3,5,15(22),16,18(21)heptaene

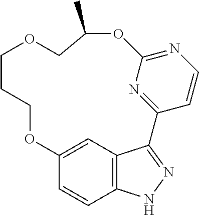 [SMILES: C[C@@H]1COCCCOc2ccc3[nH]nc(c3c2)-c2ccnc(n2)O1]

Example 55 is prepared according to the synthesis route described in general Scheme G.

### Preparation of Intermediate 307: 1-tetrahydropyran-2-ylindazol-5-ol

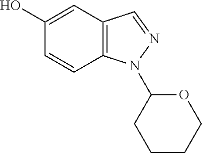 [SMILES: Oc1ccc2c(cnn2C2CCCCO2)c1]

To a solution of tert-butyl-dimethyl-(1-tetrahydropyran-2-ylindazol-5-yl)oxy-silane (intermediate 60) (40 g, 120.29 mmol) in THF (450 mL) at 0° C. was added dropwise TBAF (1M solution in THF) (157 mL, 156.38 mmol). The reaction mixture was stirred at RT for 16 h. The reaction mixture was diluted with ethyl acetate and washed with a saturated aqueous NaHCO₃ solution. The organic layer was dried over anhydrous magnesium sulfate, filtered and evaporated under reduced pressure. The residue was purified on silica gel column chromatography using heptane/ethyl acetate 100/0 to 80/20 as eluent to afford 1-tetrahydropyran-2-ylindazol-5-ol as an orange solid.

LCMS method B: [M+H]⁺=219.1, t_{R}=0.528 min

### Preparation of Intermediate 308: 5-[3-[(2S)-2-benzyloxypropoxy]propoxy]-1-tetra hydropyran-2-yl-indazole

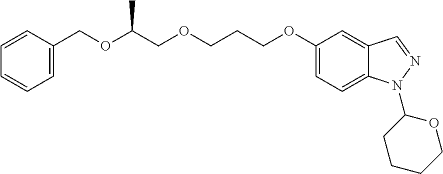 [SMILES: C[C@@H](COCCCOc1ccc2c(cnn2C2CCCCO2)c1)OCc1ccccc1]

To a solution of (S)-3-(2-(benzyloxy)propoxy)propan-1-ol (intermediate 68) (1.24 g, 5.52 mmol) in dry THF (35 mL) was sequentially added 1-tetrahydropyran-2-ylindazol-5-ol (1.448 g, 6.63 mmol) and triphenylphosphine (2.175 g, 8.29 mmol). The reaction mixture was stirred at RT for 30 min. DIAD (1.633 mL, 8.29 mmol) was added dropwise and the reaction mixture was stirred at 90° C. for 15 h. The reaction mixture was diluted with ethyl acetate and washed with brine. The organic layer was dried over anhydrous magnesium sulfate, filtered and evaporated under reduced pressure. The residue was purified by silica gel column chromatography using heptane/ethyl acetate 100/0 to 70/30 as eluent to afford 5-[3-[(2S)-2-benzyloxypropoxy]propoxy]-1-tetrahydropyran-2-yl-indazole as a yellow oil.

LCMS method B: [M+H]⁺=425.1, t_{R}=1.218 min

### Preparation of Intermediate 309: (2S)-1-[3-(1-tetrahydropyran-2-ylindazol-5-yl) oxypropoxy]propan-2-ol

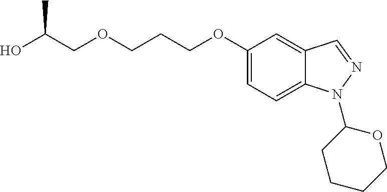 [SMILES: C[C@H](O)COCCCOc1ccc2c(cnn2C2CCCCO2)c1]

To a solution of 5-[3-[(2S)-2-benzyloxypropoxy]propoxy]-1-tetrahydropyran-2-yl-indazole (1.92 g, 4.53 mmol) in ethyl acetate (62 mL) was added palladium hydroxide on carbon (385 mg, 20% w/w). The reaction mixture was stirred under the hydrogen atmosphere at RT for 15 h. Additional palladium hydroxide on carbon (385 mg, 20% w/w) was added and reaction mixture was stirred under the hydrogen atmosphere at RT for 15 h. The reaction mixture was filtered over a pad of Celite, washed with ethyl acetate and the solvent was removed under reduced pressure. The residue was purified by silica gel column chromatography using heptane/ethyl acetate 100/0 to 40/60 as eluent to afford (2S)-1-[3-(1-tetrahydropyran-2-ylindazol-5-yl)oxypropoxy]propan-2-ol as a colorless oil.

LCMS method B: [M+H]⁺=335.0, t_{R}=0.784 min

### Preparation of Intermediate 310: tert-butyl-dimethyl-[(1S)-1-methyl-2-[3-(1-tetra hydropyran-2-ylindazol-5-yl)oxypropoxy]ethoxy]silane

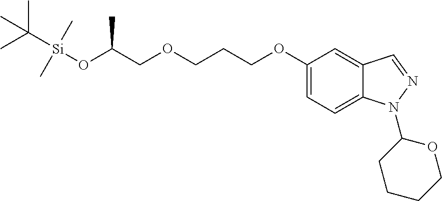 [SMILES: C[C@@H](COCCCOc1ccc2c(cnn2C2CCCCO2)c1)O[Si](C)(C)C(C)(C)C]

To a solution of (2S)-1-[3-(1-tetrahydropyran-2-ylindazol-5-yl)oxypropoxy]propan-2-ol (1.3 g, 3.88 mmol) in dichloromethane (12 mL) was added imidazole (397 mg, 5.83 mmol) and tert-butyldimethylchlorosilane (879 mg, 5.83 mmol). The reaction mixture was stirred at RT for 63 h. The reaction mixture was diluted with ethyl acetate and washed with brine. The organic layer was dried over anhydrous magnesium sulfate, filtered and evaporated under reduced pressure. The residue was purified by silica gel column chromatography using heptane/ethyl acetate 100/0 to 90/10 as eluent. to afford tert-butyl-dimethyl-[(1S)-1-methyl-2-[3-(1-tetrahydropyran-2-ylindazol-5-yl)oxypropoxy]ethoxy]silane as a colorless oil.

LCMS method B: [M+H]⁺=449.1, t_{R}=1.495 min

### Preparation of Intermediate 311: Mixture of tert-butyl-dimethyl-[(1S)-1-methyl-2-[3-[1-tetrahydropyran-2-yl-3-(4,4,5,5-tetramethyl-1,3,2-dioxaborolan-2-yl)indazol-5-yl]oxypropoxy]ethoxy]silane and [5-[3-[(2S)-2-[tert-butyl(dimethyl) silyl]oxypropoxy] propoxy]-1-tetrahydropyran-2-yl-indazol-3-yl]boronic acid

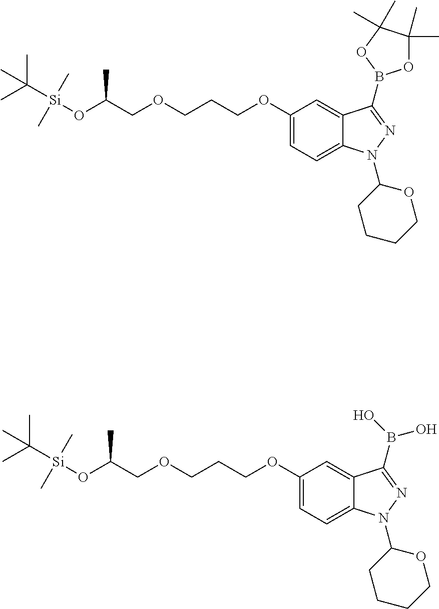

To a solution of tert-butyl-dimethyl-[(1S)-1-methyl-2-[3-(1-tetrahydropyran-2-ylindazol-5-yl) oxypropoxy]ethoxy] silane (1.36 g, 3.03 mmol) in dry MTBE (15 mL) under N₂ in a pressure flask. was added bis(pinacolato)diboron (1.155 g, 4.54 mmol), 4,4′-di-tert-butyl-2,2′-bipyridine (8 mg, 0.03 mmol) and 1,5-cyclooctadiene)(methoxy)iridium(I) dimer (40 mg, 0.061 mmol). The reaction mixture was stirred at 90° C. for 15 h. The reaction mixture was cooled to RT and filtered through a pad of Celite, washed with ethyl acetate and the solvent was removed under reduced pressure to afford tert-butyl-dimethyl-[(1S)-1-methyl-2-[3-[1-tetrahydropyran-2-yl-3-(4,4,5,5-tetramethyl-1,3,2-dioxaborolan-2-yl)indazol-5-yl]oxypropoxy]ethoxy]silane and [5-[3-[(2S)-2-[tert-butyl(dimethyl)silyl]oxypropoxy]propoxy]-1-tetrahydropyran-2-yl-indazol-3-yl]boronic acid as a yellow oil which was used in the next step without further purification.

LCMS method B: [M+H]⁺=493.1, t_{R}=1.358 min

### Preparation of Intermediate 312: tert-butyl-dimethyl-[(1S)-1-methyl-2-[3-[3-(2-methylsulfanylpyrimidin-4-yl)-1-tetrahydropyran-2-yl-indazol-5-yl]oxypropoxy] ethoxy]silane

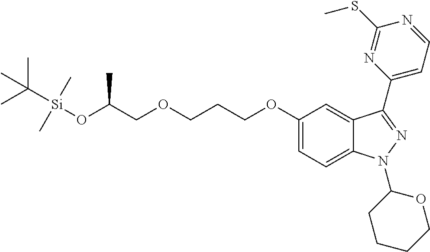 [SMILES: CSc1nccc(-c2nn(C3CCCCO3)c3ccc(OCCCOC[C@H](C)O[Si](C)(C)C(C)(C)C)cc23)n1]

To a solution of a mixture 5-(3-((S)-2-((tert-butyldimethylsilyl)oxy)propoxy)propoxy)-1-(tetrahydro-2H-pyran-2-yl)-3-(4,4,5,5-tetramethyl-1,3,2-dioxaborolan-2-yl)-1H-indazole and [5-[3-[(2S)-2-[tert-butyl(dimethyl)silyl]oxypropoxy]propoxy]-1-tetrahydropyran-2-yl-indazol-3-yl]boronic acid (crude, 3.031 mmol) in 1,4-dioxane (28.3 mL) and water (1.7 mL) under N₂ in a pressure flask was added 4-chloro-2-methylsulfanylpyrimidine (353 mg, 3.031 mmol), potassium phosphate tribasic (1.93 g, 9.09 mmol), XPhos (144 mg, 0.303 mmol) and tetrakis(triphenylphosphine)palladium(0) (176 mg, 0.152 mmol) were added. The reaction mixture was stirred at 90° C. for 2 h. The reaction mixture was filtered through a pad of Celite and washed with ethyl acetate. The organic layer was washed with water and brine, dried over. anhydrous magnesium sulfate, filtered and evaporated under reduced pressure. The residue was purified by silica gel column chromatography using heptane/ethyl acetate 100/0 to 90/10 as eluent to afford tert-butyl-dimethyl-[(1S)-1-methyl-2-[3-[3-(2-methylsulfanylpyrimidin-4-yl)-1-tetrahydropyran-2-yl-indazol-5-yl]oxypropoxy]ethoxy]silane as a yellow oil.

LCMS method B: [M+H]⁺=573.0, t_{R}=1.670 min

### Preparation of Intermediate 313: (2S)-1-[3-[3-(2-methylsulfanylpyrimidin-4-yl)-1-tetrahydropyran-2-yl-indazol-5-yl]oxypropoxy]propan-2-ol

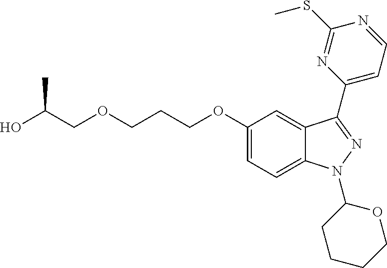 [SMILES: CSc1nccc(-c2nn(C3CCCCO3)c3ccc(OCCCOC[C@H](C)O)cc23)n1]

To a solution of tert-butyl-dimethyl-[(1S)-1-methyl-2-[3-[3-(2-methylsulfanylpyrimidin-4-yl)-1-tetrahydropyran-2-yl-indazol-5-yl]oxypropoxy]ethoxy]silane (1.4 g, 2.44 mmol) in THF (15 mL) at 0° C. was added TBAF (1M solution in THF) (3.66 mL, 3.66 mmol) and the reaction mixture was stirred at RT for 15 h. The reaction mixture was diluted with ethyl acetate and washed with a saturated aqueous NaHCO₃ solution and brine. The organic layer was dried over anhydrous magnesium sulfate, filtered and evaporated reduced pressure. The residue was purified by silica gel column chromatography using heptane/ethyl acetate 100/0 to 20/80 as eluent to afford (2S)-1-[3-[3-(2-methylsulfanylpyrimidin-4-yl)-1-tetrahydropyran-2-yl-indazol-5-yl]oxypropoxy]propan-2-ol as a white solid.

LCMS method B: [M+H]⁺=459.0, t_{R}=1.087 min

### Preparation of Intermediate 314: (2S)-1-[3-[3-(2-methylsulfonylpyrimidin-4-yl)-1-tetrahydropyran-2-yl-indazol-5-yl]oxypropoxy]propan-2-ol

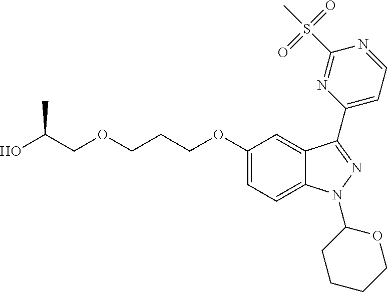 [SMILES: C[C@H](O)COCCCOc1ccc2c(c1)c(-c1ccnc(S(C)(=O)=O)n1)nn2C1CCCCO1]

To a solution of (2S)-1-[3-[3-(2-methylsulfanylpyrimidin-4-yl)-1-tetrahydropyran-2-yl-indazol-5-yl]oxypropoxy]propan-2-ol (978 mg, 2.13 mmol) in dichloromethane (22 mL) at 0° C. was added 3-chloroperbenzoic acid (1.22 g, 5.33 mmol) and the reaction mixture was stirred at RT for 2 h. The reaction mixture was diluted with dichloromethane and washed with a saturated aqueous NaHCO₃ solution. The aqueous layer was extracted with dichloromethane and the combined organic layers were washed with brine, dried over anhydrous magnesium sulfate, filtered and evaporated under reduced pressure. The residue was purified by silica gel column chromatography using heptane/ethyl acetate 100/0 to 20/80 as eluent to afford (2S)-1-[3-[3-(2-methylsulfonylpyrimidin-4-yl)-1-tetrahydropyran-2-yl-indazol-5-yl]oxypropoxy]propan-2-ol as a yellow solid.

LCMS method B: [M+H]⁺=491.0, t_{R}=0.886 min

### Preparation of Intermediate 315: (8R)-8-methyl-19-(oxan-2-yl)-7,10,14-trioxa-5,19,20,23-tetraazatetracyclo[13.5.2.1^{2,6}.0^{18,21}]tricosa-1(20),2(23),3,5,15(22),16, 18(21)-heptaene

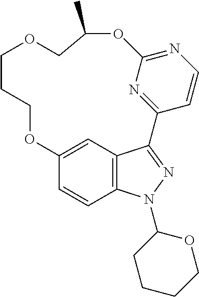 [SMILES: C[C@@H]1COCCCOc2ccc3c(c2)c(nn3C2CCCCO2)-c2ccnc(n2)O1]

A solution of (2S)-1-[3-[3-(2-methylsulfonylpyrimidin-4-yl)-1-tetrahydropyran-2-yl-indazol-5-yl]oxypropoxy]propan-2-ol (751 mg, 1.531 mmol) in dry THF (77 mL) was added dropwise under nitrogen atmosphere to a stirred solution of lithium bis(trimethylsilyl)amide (1M solution in THF) (2.29 mL, 2.29 mmol) in dry THF (77 mL). The reaction mixture was stirred at RT for 1 h. The reaction mixture was cooled to 0° C., diluted with ethyl acetate, quenched with a saturated aqueous ammonium chloride solution and washed with brine. The organic layer was dried over anhydrous magnesium sulfate, filtered and evaporated under reduced pressure. The residue was purified by silica gel column chromatography using heptane/ethyl acetate 100/0 to 85/15 as eluent to afford (8R)-8-methyl-19-(oxan-2-yl)-7,10,14-trioxa-5,19,20,23-tetraaza tetracyclo[13.5.2.1^{2,6}.0^{18,21}]tricosa-1(20),2(23),3,5,15(22),16,18(21)-heptaene as a white solid.

LCMS method B: [M+H]⁺=411.0, t_{R}=1.178 min

### Preparation of Example 55: (8R)-8-methyl-7,10,14-trioxa-5,19,20,23-tetraazatetra cyclo[13.5.2.1^{2,6}.0^{18,21}]tricosa-1(20),2(23),3, 5,15(22),16,18(21)heptaene

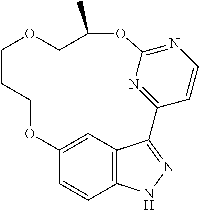 [SMILES: C[C@@H]1COCCCOc2ccc3[nH]nc(c3c2)-c2ccnc(n2)O1]

Hydrogen chloride (4M solution in dioxane) (12 mL) was added to (8R)-8-methyl-19-(oxan-2-yl)-7,10,14-trioxa-5,19,20,23-tetraazatetracyclo[13.5.2.1^{2,6}.0^{18,21}]tricosa-1(20),2(23),3, 5, 15(22),16,18(21)-heptaene (466 mg, 1.135 mmol). The reaction mixture was stirred at RT for 15 h. The solvent was removed under reduced pressure and the residue was basified to pH8 using a saturated aqueous NaHCO₃ solution. The aqueous layer was extracted with dichloromethane. The combined organic layers were dried over anhydrous magnesium sulfate, filtered and evaporated under reduced pressure. The residue was purified by silica gel column chromatography using heptane/ethyl acetate 100/0 to 50/50 as eluent to afford (8R)-8-methyl-7,10,14-trioxa-5,19,20,23-tetraazatetracyclo[13.5.2.1^{2,6}.0^{18,21}]tricosa-1(20),2(23),3,5,15(22), 16,18(21)heptaene as a solid.

LCMS method E: [M+H]+=327.1, t_{R}=3.219 min

LCMS method D: [M+H]+=327.0, t_{R}=2.532 min 1H NMR (400 MHz, d6-DMSO) 13.66 (s, 1H), 8.58 (d, J=5.2 Hz, 1H), 8.03 (d, J=2.4 Hz, 1H), 7.75 (d, J=5.1 Hz, 1H), 7.54 (d, J=9.0 Hz, 1H), 7.04 (dd, J=9.0, 2.4 Hz, 1H), 5.18 (dqd, J=9.8, 6.3, 3.4 Hz, 1H), 4.46 (ddd, J=12.1, 8.3, 5.9 Hz, 1H), 4.37-4.18 (m, 2H), 3.72 (ddd, J=11.8, 9.3, 2.9 Hz, 1H), 3.55 (ddd, J=11.0, 5.8, 3.3 Hz, 1H), 3.32-3.27 (m, 1H), 2.42-2.22 (m, 1H), 1.68 (ddtd, J=14.0, 8.3, 5.6, 2.9 Hz, 1H), 1.47 (d, J=6.3 Hz, 3H) ppm.

### Example 56: (13R)-13-methyl-7,10,14-trioxa-19,20,23-triazatetracyclo[13.5.2.1^{2,6}.0^{18,21}] tricosa-1(20),2(23),3,5,15(22),16,18(21)-heptaene

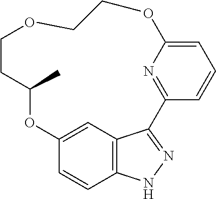 [SMILES: C[C@@H]1CCOCCOc2cccc(n2)-c2n[nH]c3ccc(cc23)O1]

Example 56 is prepared according to the synthesis route described in general Scheme D, following the same synthesis procedures as for Example 28.

### Preparation of Intermediate 316: (13R)-13-methyl-19-(oxan-2-yl)-7,10,14-trioxa-19,20,23-triazatetracyclo[13.5.2.1^{2,6}.0^{18,21}]tricosa-1(20),2(23),3,5,15(22),16,18(21)-heptaene

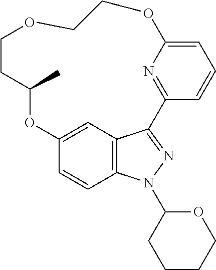 [SMILES: C[C@@H]1CCOCCOc2cccc(n2)-c2nn(C3CCCCO3)c3ccc(cc23)O1]

To a suspension of sodium hydride (60% dispersion in mineral oil) (29 mg, 0.73 mmol) in anhydrous DMF (75 mL) at RT was added dropwise [(1S)-3-[2-[[6-[5-[tert-butyl(dimethyl) silyl]oxy-1-tetrahydropyran-2-yl-indazol-3-yl]-2-pyridyl]oxy]ethoxy]-1-methyl-propyl] methanesulfonate (150 mg, 0.24 mmol) in DMF (75 mL). The reaction mixture was stirred at RT overnight. The solvent was removed under reduced pressure, the residue was dissolved in ethyl acetate and water and the organic layer was washed with a saturated aqueous ammonium chloride solution and water. The organic layer was dried over anhydrous sodium sulfate, filtered and evaporated under reduced pressure to afford (13R)-13-methyl-19-(oxan-2-yl)-7,10,14-trioxa-19,20,23-triazatetracyclo[13.5.2.1^{2,6}.0^{18,21}]tricosa-1(20),2(23),3,5,15(22),16,18(21)-heptaene as a pink solid which was used in next step without further purification.

LCMS method F: [M+H]⁺=410.3, t_{R}=3.47 min

### Preparation of Example 56: (13R)-13-methyl-7,10,14-trioxa-19,20,23-triazatetra cyclo[13.5.2.1^{2,6}.0^{18,21}]tricosa-1(20),2(23),3,5,15(22),16,18(21)-heptaene

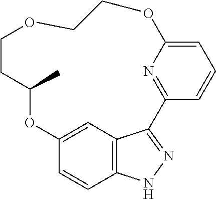 [SMILES: C[C@@H]1CCOCCOc2cccc(n2)-c2n[nH]c3ccc(cc23)O1]

To a solution of (13R)-13-methyl-19-(oxan-2-yl)-7,10,14-trioxa-19,20,23-triazatetracyclo [13.5.2.1^{2,6}.0^{18,21}]tricosa-1(20),2(23),3,5,15(22),16,18(21)-heptaene (100 mg, 0.24 mmol) in methanol (10 mL) and water (1.5 mL) was added p-toluenesulfonic acid monohydrate (232 mg, 1.22 mmol) and the reaction mixture was stirred at 65° C. for 2 h. The reaction mixture was concentrated under reduced pressure and the residue was neutralized by addition of a saturated aqueous NaHCO₃ solution. The residue was diluted with ethyl acetate and the phases were separated. The aqueous layer was extracted with ethyl acetate. The combined organic layers were washed with a saturated aqueous NaHCO₃ solution, water and brine, dried over anhydrous sodium sulfate, filtered and evaporated under reduced pressure. The residue was purified by silica gel column chromatography using cyclohexane/ethyl acetate 100/0 to 70/30 as eluent. The resulting oil was crystallized in methanol to afford (13R)-13-methyl-7,10,14-trioxa-19,20,23-triazatetracyclo[13.5.2.1^{2,6}.0^{18,21}]tricosa-1(20),2(23),3,5,15(22),16,18(21)-heptaene as a powder.

LCMS method F: [M+H]⁺=326.3, t_{R}=2.74 min

LCMS method G: [M+H]⁺=326.3, t_{R}=2.59 min

1H NMR (400 MHz, d6-DMSO) 13.24 (1H, s), 8.15 (1H, d, J=2.1 Hz), 7.80-7.77 (2H, m), 7.49-7.46 (1H, m), 6.99 (1H, dd, J=2.3, 8.9 Hz), 6.75-6.72 (1H, m), 5.06-4.99 (1H, m), 4.69-4.62 (1H, m), 4.26-4.14 (2H, m), 3.75-3.60 (3H, m), 2.41-2.34 (1H, m), 1.40-1.37 (4H, m) ppm.

### Example 57: (12S)-12-methyl-9,13-dioxa-3,5,18,19-tetraazatetracyclo [12.5.2.1^{2,5}.0^{17,20}] docosa-1(19),2(22),3,14(21),15,17(20)-hexaene

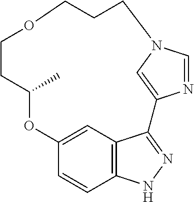 [SMILES: C[C@H]1CCOCCCn2cnc(c2)-c2n[nH]c3ccc(cc23)O1]

Example 57 is prepared according to the synthesis route described in general Scheme D.

### Preparation of Intermediate 317: 3-(4-bromoimidazol-1-yl)propoxy-tert-butyl-dimethyl-silane

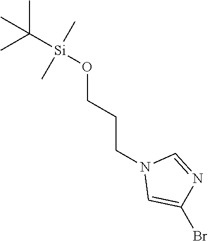 [SMILES: CC(C)(C)[Si](C)(C)OCCCn1cnc(Br)c1]

A mixture of 4-bromo-1H-imidazole (14 g, 95.25 mmol), (3-bromopropoxy)(tert-butyl) dimethylsilane (22.13 ml, 95.25 mmol), potassium carbonate (26.33 g, 190.51 mmol) and potassium iodide (18.97 g, 114.31 mmol) in acetone (285 mL) was stirred at 60° C. for 16 h. Additional (3-bromopropoxy)(tert-butyl)dimethylsilane (11.06 ml, 47.62 mmol), potassium carbonate (13.16 g, 95.25 mmol) and potassium iodide (9.48 g, 57.15 mmol) were added and the mixture was stirred at 60° C. for 16 h. The reaction mixture was diluted with dichloromethane and filtered. The filtrate was diluted with dichloromethane, washed with water and extracted with dichloromethane. The combined organic layers were dried over anhydrous magnesium sulfate, filtered and evaporated under reduced pressure. The residue was purified by silica gel column chromatography using heptane/ethyl acetate 100/0 to 75/25 as eluent to afford 3-(4-bromoimidazol-1-yl)propoxy-tert-butyl-dimethyl-silane as a yellow oil.

LCMS method B: [M+H]⁺=319.0-321.0, t_{R}=1.123 min

### Preparation of Intermediate 318: 3-(4-bromoimidazol-1-yl)propan-1-ol

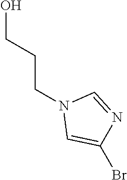

Hydrogen chloride (4M in dioxane) (143 mL) was added to 3-(4-bromoimidazol-1-yl)propoxy-tert-butyl-dimethyl-silane (15.3 g, 47.91 mmol) at 0° C. and the reaction mixture was stirred at RT for 1.5 h. The solvent was removed under reduced pressure. The mixture was diluted with dichloromethane, basified with a saturated aqueous NaHCO₃ solution and extracted with dichloromethane and chloroform/isopropyl alcohol (3:1). The combined organic layers were dried over anhydrous magnesium sulfate, filtered and evaporated under reduced pressure. The residue was purified by silica gel column chromatography using heptane/ethyl acetate 100/0 to 80/20 as eluent to afford 3-(4-bromoimidazol-1-yl)propan-1-ol as a beige solid.

LCMS method B: [M+H]⁺=205.0-207.0, t_{R}=0.160 min

### Preparation of Intermediate 319: 1-[3-[(3R)-3-benzyloxybutoxy]propyl]-4-bromo-imidazole

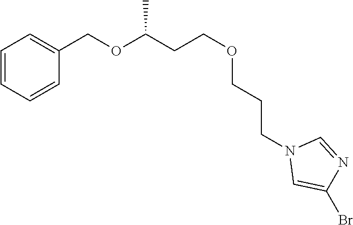 [SMILES: C[C@H](CCOCCCn1cnc(Br)c1)OCc1ccccc1]

3-(4-bromoimidazol-1-yl)propan-1-ol (3 g, 14.63 mmol) and potassium iodide (2.42 g, 14.63 mmol) in dry DMF (36.5 mL) at 0° C. under nitrogen atmosphere was added sodium hydride (60% dispersion in mineral oil) (1.17 g, 29.26 mmol). The reaction mixture was stirred at 0° C. for 30 min then [(1R)-3-bromo-1-methyl-propoxy]methylbenzene (intermediate 166) (5.33 g, 21.94 mmol) in dry DMF (36.5 mL) under nitrogen atmosphere was added. The reaction mixture was stirred at RT for 5 h. The reaction mixture was diluted with dichloromethane, water was added and the aqueous layer was extracted with dichloromethane. The combined organic layers were washed with brine and extracted with dichloromethane. The combined organic layers were dried over anhydrous magnesium sulfate, filtered and evaporated under reduced pressure. The residue was purified by silica gel column chromatography using heptane/ethyl acetate 100/0 to 70/30 as eluent to afford 1-[3-[(3R)-3-benzyloxybutoxy]propyl]-4-bromo-imidazole as a yellow oil.

LCMS method E: [M+H]⁺=367.0-369.0, t_{R}=3.511 min

### Preparation of Intermediate 320: [3-[1-[3-[(3R)-3-benzyloxybutoxy]propyl]imidazol-4-yl]-1-tetrahydropyran-2-yl-indazol-5-yl]oxy-tert-butyl-dimethyl-silane

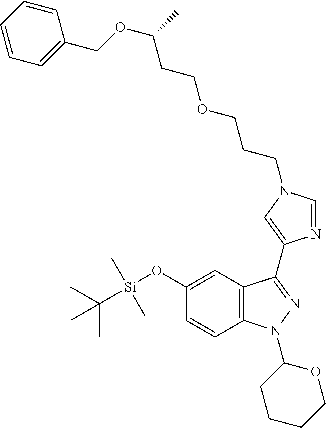 [SMILES: C[C@H](CCOCCCn1cnc(-c2nn(C3CCCCO3)c3ccc(O[Si](C)(C)C(C)(C)C)cc23)c1)OCc1ccccc1]

To a degassed solution of 1-[3-[(3R)-3-benzyloxybutoxy]propyl]-4-bromo-imidazole (1.18 g, 3.22 mmol), tert-butyl-dimethyl-[1-tetrahydropyran-2-yl-3-(4,4,5,5-tetramethyl-1,3,2-dioxa borolan-2-yl)indazol-5-yl]oxy-silane (2.22 g, 4.83 mmol), potassium phosphate tribasic (2.05 g, 9.66 mmol) in dioxane (37 mL) and water (4 mL) were added XPhos (153 mg, 0.32 mmol) and tetrakis(triphenylphosphine)palladium(0) (186 mg, 0.16 mmol). The reaction mixture was stirred at 90° C. for 4 h. The mixture was degassed and additional tert-butyl-dimethyl-[1-tetrahydropyran-2-yl-3-(4,4,5,5-tetramethyl-1,3,2-dioxaborolan-2-yl)indazol-5-yl]oxy-silane (294 mg, 0.64 mmol), XPhos (30 mg, 0.06 mmol) and tetrakis(triphenylphosphine)palladium(0) (37 mg, 0.03 mmol) were added. The reaction mixture was stirred at 90° C. overnight. The reaction mixture was filtered and washed with ethyl acetate. Water was added to the filtrate and the aqueous layer was extracted with ethyl acetate. The combined organic layers were washed with brine, dried over anhydrous sodium sulfate and evaporated under reduced pressure. The residue was purified by silica gel column chromatography using cyclohexane/ethyl acetate 100/0 to 60/40 as eluent to afford [3-[1-[3-[(3R)-3-benzyloxybutoxy]propyl]imidazol-4-yl]-1-tetrahydropyran-2-yl-indazol-5-yl]oxy-tert-butyl-dimethyl-silane as a pink oil.

LCMS method F: [M+H]⁺=619.4, t_{R}=2.95 min

### Preparation of Intermediate 321: (2R)-4-[3-[4-[5-[tert-butyl(dimethyl)silyl]oxy-1-tetrahydropyran-2-yl-indazol-3-yl]imidazol-1-yl]propoxy]butan-2-ol

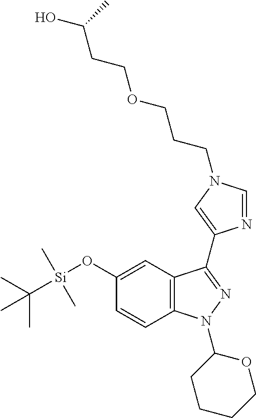 [SMILES: C[C@@H](O)CCOCCCn1cnc(-c2nn(C3CCCCO3)c3ccc(O[Si](C)(C)C(C)(C)C)cc23)c1]

To a solution of [3-[1-[3-[(3R)-3-benzyloxybutoxy]propyl]imidazol-4-yl]-1-tetrahydropyran-2-yl-indazol-5-yl]oxy-tert-butyl-dimethyl-silane (393 mg, 0.64 mmol) in dichloromethane (12.7 mL) and phosphate buffer (pH 7) (0.65 mL) at RT was added 2,3-dichloro-5,6-dicyano-p-benzoquinone (289 mg, 1.27 mmol). The reaction was stirred at RT overnight then 40° C. for 7 h. Additional 2,3-dichloro-5,6-dicyano-p-benzoquinone (144 mg, 0.64 mmol) was added and the reaction mixture was stirred at 40° C. for 48 h. Additional 2,3-dichloro-5,6-dicyano-p-benzoquinone (DDQ) (289 mg, 1.27 mmol) was added and the mixture was stirred at 40° C. for 4 h. The reaction mixture was cooled to RT and a saturated aqueous Na₂CO₃ solution and dichloromethane were added. The aqueous layer was extracted with dichloromethane and the combined organic layers were washed with water and brine, dried over anhydrous sodium sulfate, filtered and evaporated under reduced pressure. The residue was purified by silica gel column chromatography using dichloromethane/methanol 100/0 to 95/5 as eluent to afford (2R)-4-[3-[4-[5-[tert-butyl(dimethyl)silyl]oxy-1-tetrahydropyran-2-yl-indazol-3-yl]imidazol-1-yl]propoxy]butan-2-ol as a brown oil.

LCMS method F: [M+H]⁺=529.3, t_{R}=2.45 min

### Preparation of Intermediate 322: [(1R)-3-[3-[4-(5-hydroxy-1-tetrahydropyran-2-yl-indazol-3-yl)imidazol-1-yl]propoxy]-1-methyl-propyl] methanesulfonate

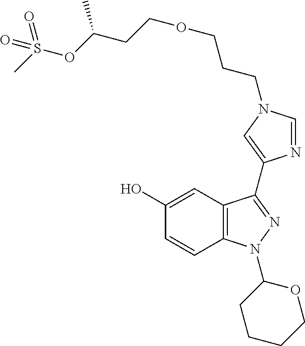 [SMILES: C[C@H](CCOCCCn1cnc(-c2nn(C3CCCCO3)c3ccc(O)cc23)c1)OS(C)(=O)=O]

To a solution of (2R)-4-[3-[4-[5-[tert-butyl(dimethyl)silyl]oxy-1-tetrahydropyran-2-yl-indazol-3-yl]imidazol-1-yl]propoxy]butan-2-ol (156 mg, 0.3 mmol) and triethylamine (84 μL, 0.6 mmol) in dichloromethane (3.8 mL) at 0° C. was added dropwise methanesulfonyl chloride (30 μL, 0.39 mmol). The reaction mixture was stirred at RT for 4 h. additional methanesulfonyl chloride (20 μL, 0.26 mmol) was added and the reaction mixture was stirred at RT for 2 h. Additional triethylamine (84 μL, 0.6 mmol) and methanesulfonyl chloride (30 μL, 0.39 mmol) were added and the mixture was stirred at RT overnight. The mixture was diluted with water and extracted with dichloromethane. The combined organic layers were washed with brine, dried over anhydrous sodium sulfate, filtered and evaporated under reduced pressure to afford [(1R)-3-[3-[4-(5-hydroxy-1-tetrahydropyran-2-yl-indazol-3-yl)imidazol-1-yl]propoxy]-1-methyl-propyl]methanesulfonate as a brown paste which was used in the next step without further purification.

LCMS method F: [M+H]⁺=493.4, t_{R}=1.59 min

### Preparation of Intermediate 323: (12S)-12-methyl-18-(oxan-2-yl)-9,13-dioxa-3,5,18,19-tetraazatetracyclo[12.5.2.1^{2,5}.0^{17,20}]docosa-1(19),2(22),3,14(21),15,17(20)-hexaene

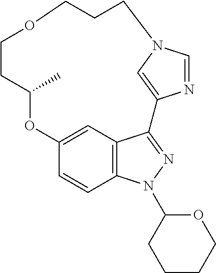 [SMILES: C[C@H]1CCOCCCn2cnc(c2)-c2nn(C3CCCCO3)c3ccc(cc23)O1]

To a degassed suspension of sodium hydride (60% dispersion in mineral oil) (62 mg, 1.56 mmol) in dry DMF (93 mL) at RT was added dropwise, [(1R)-3-[3-[4-(5-hydroxy-1-tetrahydropyran-2-yl-indazol-3-yl)imidazol-1-yl]propoxy]-1-methyl-propyl] methanesulfonate (126 mg, 0.26 mmol) in DMF (93 mL). The reaction mixture was stirred at RT overnight. The reaction mixture was quenched with water and was concentrated under reduced pressure. The residue was dissolved in ethyl acetate and diluted with water. The aqueous layer was extracted with ethyl acetate. The combined organic layers were washed with brine, dried over anhydrous sodium sulfate, filtered and evaporated under reduced pressure. The residue was purified by silica gel column chromatography using dichloromethane/methanol 100/0 to 95/5 as eluent to afford (12S)-12-methyl-18-(oxan-2-yl)-9,13-dioxa-3,5,18,19-tetraazatetracyclo[12.5.2.1^{2,5}.0^{17,20}]docosa-1(19),2(22),3,14(21),15,17(20)-hexaene as a yellow solid.

LCMS method F: [M+H]⁺=397.2, t_{R}=1.91 min

### Preparation of Example 57: (12S)-12-methyl-9,13-dioxa-3,5,18,19-tetraazatetracyclo [12.5.2.1^{2,5}.0^{17,20}]docosa-1(19),2(22),3,14(21),15,17(20)-hexaene

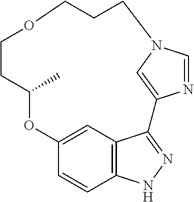 [SMILES: C[C@H]1CCOCCCn2cnc(c2)-c2n[nH]c3ccc(cc23)O1]

To a solution of (12S)-12-methyl-18-(oxan-2-yl)-9,13-dioxa-3,5,18,19-tetraazatetracyclo [12.5.2.1^{2,5}.0^{17,20}]docosa-1(19),2(22),3,14(21),15,17(20)-hexaene (11 mg, 0.028 mmol) in methanol (470 μL) and water (80 μL) was added p-toluenesulfonic acid monohydrate (27 mg, 0.14 mmol). The reaction mixture was stirred at 65° C. overnight. The reaction mixture was diluted with dichloromethane and a saturated aqueous NaHCO₃ solution. The aqueous layer was extracted with dichloromethane. The combined organic layers were washed brine, dried over anhydrous sodium sulfate, filtered and evaporated under reduced pressure. The residue was triturated with diethyl ether, filtered and dried to afford (12S)-12-methyl-9,13-dioxa-3,5,18,19-tetraazatetracyclo[12.5.2.1^{2,5}.0^{17,20}]docosa-1(19),2(22),3,14(21),15,17(20)-hexaene as a cream solid.

LCMS method F: [M+H]⁺=313, t_{R}=1.49 min

LCMS method G: [M+H]⁺=313, t_{R}=2.04 min 1H NMR (400 MHz, d6-DMSO) 12.67 (1H, s), 8.05 (1H, d, J=1.1 Hz), 7.70 (1H, d, J=1.1 Hz), 7.50 (1H, d, J=2 Hz), 7.37 (1H, d, J=8.5 Hz), 6.91 (1H, dd, J=2.3, 8.9 Hz), 4.50 (1H, m), 4.37 (1H, dd, J=7.5, 14.3 Hz), 4.09 (1H, m), 3.69 (3H, m), 3.54 (1H, t, J=10.39 Hz), 3.32 (1H, m), 2.20 (1H, m), 1.95 (1H, m), 1.5 (1H, m), 1.41 (3H, d, J=6 Hz) ppm.

### Example 58: (9R,13S)-9,13-dimethyl-7,10,14-trioxa-4,19,20,23-tetraazatetracyclo[13.5.2.1^{2,6}.0^{18,21}]tricosa-1(20),2(23),3,5,15(22),16,18(21)-heptaene

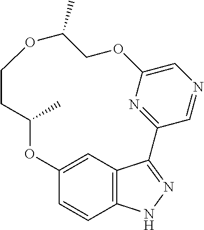 [SMILES: C[C@@H]1COc2cncc(n2)-c2n[nH]c3ccc(cc23)O[C@@H](C)CCO1]

Example 58 is prepared according to the synthesis route described in general Scheme D, following the same synthesis procedures as for Example 51.

### Preparation of Intermediate 324: (9R,13S)-9,13-dimethyl-19-(oxan-2-yl)-7,10,14-trioxa-4,19,20,23-tetraazatetracyclo[13.5.2.1^{2,6}.0^{18,21}]tricosa-1(20),2(23),3,5,15(22), 16,18(21)-heptaene

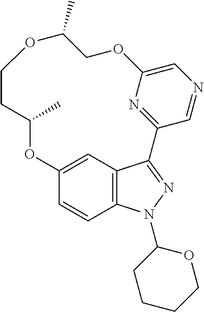 [SMILES: C[C@@H]1COc2cncc(n2)-c2nn(C3CCCCO3)c3ccc(cc23)O[C@@H](C)CCO1]

To a suspension of sodium hydride (60% dispersion in mineral oil) (22 mg, 0.54 mmol) in anhydrous DMF (27 mL) at RT was added dropwise [(1R)-3-[(1R)-2-[6-[5-[tert-butyl (dimethyl)silyl]oxy-1-tetrahydropyran-2-yl-indazol-3-yl]pyrazin-2-yl]oxy-1-methyl-ethoxy]-1-methyl-propyl] methanesulfonate (114 mg, 0.18 mmol) in DMF (27 mL). The reaction mixture was stirred at RT over 72 h. The reaction mixture was quenched with water and concentrated under reduced pressure. The residue was diluted with brine and extracted with ethyl acetate. The combined organic layers were dried over anhydrous sodium sulfate, filtered and evaporated under reduced pressure. The crude product was purified by silica gel column chromatography using cyclohexane/ethyl acetate 100/0 to 80/20 as eluent to afford (9R,13S)-9,13-dimethyl-19-(oxan-2-yl)-7,10,14-trioxa-4,19,20,23-tetraazatetracyclo[13.5.2.1^{2,6}.0^{18,21}] tricosa-1(20),2(23),3,5,15(22),16,18(21)-heptaene as a white solid.

LCMS method F: [M+H]⁺=425.3, t_{R}=3.22 min

### Preparation of Example 58: (9R,13S)-9,13-dimethyl-7,10,14-trioxa-4,19,20,23-tetra azatetracyclo[13.5.2.1^{2,6}.0^{18,21}]tricosa-1(20),2(23),3,5,15(22),16,18(21)-heptaene

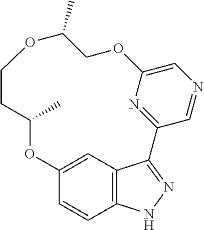 [SMILES: C[C@@H]1COc2cncc(n2)-c2n[nH]c3ccc(cc23)O[C@@H](C)CCO1]

To a solution of (9R,13S)-9,13-dimethyl-19-(oxan-2-yl)-7,10,14-trioxa-4,19,20,23-tetraaza tetracyclo[13.5.2.1^{2,6}.0^{18,21}]tricosa-1(20),2(23),3,5,15(22),16,18(21)-heptaene (47 mg, 0.11 mmol) in methanol (7 mL) and water (1 mL) was added p-toluenesulfonic acid monohydrate (105 mg, 0.55 mmol). The reaction mixture was stirred at 80° C. overnight. The reaction mixture was concentrated under reduced pressure and the residue was neutralized by addition of a saturated aqueous NaHCO₃ solution. Ethyl acetate was added, the layers were separated and the aqueous layer was extracted with ethyl acetate. The combined organic layers were washed with brine, dried over anhydrous sodium sulfate, filtered and evaporated under reduced pressure. The residue was recrystallized from acetonitrile, filtered and dried to afford (9R,13S)-9,13-dimethyl-7,10,14-trioxa-4,19,20,23-tetraazatetracyclo[13.5.2.1^{2,6}.0^{18,21}]tricosa-1(20), 2(23),3,5,15(22),16,18(21)-heptaene as a solid.

LCMS method F: [M+H]⁺=341.3, t_{R}=2.51 min

LCMS method G: [M+H]⁺=341.3, t_{R}=2.49 min 1H NMR (400 MHz, d6-DMSO) 13.35 (1H, s), 8.82 (1H, d, J=0.6 Hz), 8.58 (1H, d, J=2.3 Hz), 8.20 (1H, s), 7.50-7.47 (1H, m), 6.98 (1H, dd, J=2.4, 9.0 Hz), 5.53-5.49 (1H, m), 4.65-4.59 (1H, m), 4.14 (1H, dd, J=3.1, 12.4 Hz), 3.91-3.87 (2H, m), 3.20 (1H, t, J=10.8 Hz), 2.22-2.14 (1H, m), 1.45-1.42 (4H, m), 1.29 (3H, d, J=6.3 Hz) ppm.

### Example 59: (9S,13S)-9,13-dimethyl-7,10,14-trioxa-5,19,20,23-tetraazatetracyclo [13.5.2.1^{2,6}.0^{18,21}]tricosa-1(20),2(23),3,5,15(22),16,18(21)-heptaene

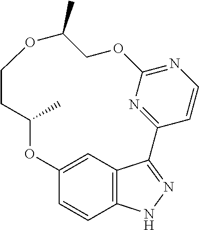 [SMILES: C[C@H]1COc2nccc(n2)-c2n[nH]c3ccc(cc23)O[C@@H](C)CCO1]

Example 59 is prepared according to the synthesis route described in general Scheme C.

### Preparation of Intermediate 325: [3-[2-[(2S)-2-[(3R)-3-benzyloxybutoxy]propoxy] pyrimidin-4-yl]-1-tetrahydropyran-2-yl-indazol-5-yl]oxy-tert-butyl-dimethyl-silane

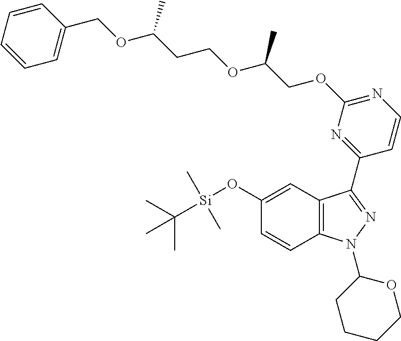 [SMILES: C[C@H](CCO[C@@H](C)COc1nccc(-c2nn(C3CCCCO3)c3ccc(O[Si](C)(C)C(C)(C)C)cc23)n1)OCc1ccccc1]

To a solution of (2S)-2-[(3R)-3-benzyloxybutoxy]propan-1-ol (intermediate 279) (400 mg, 1.681 mmol) in dry THF (10 mL) at 0° C. was added portionwise sodium hydride (60% dispersion mineral in oil) (81 mg, 2.017 mmol). The reaction mixture was stirred at 0° C. for 15 min and a solution of tert-butyl-dimethyl-[3-(2-methylsulfonylpyrimidin-4-yl)-1-tetrahydropyran-2-yl-indazol-5-yl]oxy-silane (intermediate 63) (820 mg, 1.681 mmol) in dry THF (7 mL) was added dropwise. The reaction mixture was stirred at 0° C. for 10 min then allowed to RT and stirred for 16 h. A saturated aqueous ammonium chloride solution was added and the mixture was diluted with ethyl acetate. The phases were separated and the aqueous layer was extracted with ethyl acetate. The combined organic layers were washed with water and brine, dried over anhydrous sodium sulfate, filtered and evaporated under reduced pressure. The residue was purified by silica gel column chromatography using cyclohexane/ethyl acetate 100/0 to 60/40 as eluent to afford [3-[2-[(2S)-2-[(3R)-3-benzyloxybutoxy]propoxy]pyrimidin-4-yl]-1-tetrahydropyran-2-yl-indazol-5-yl]oxy-tert-butyl-dimethyl-silane as a yellow oil.

LCMS method M: [M+H]⁺=647.4, t_{R}=5.88 min

### Preparation of Intermediate 326: (2R)-4-[(1S)-2-[4-[5-[tert-butyl(dimethyl)silyl]oxy-1-tetrahydropyran-2-yl-indazol-3-yl]pyrimidin-2-yl]oxy-1-methyl-ethoxy]butan-2-ol

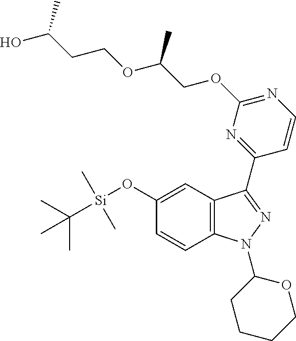 [SMILES: C[C@@H](O)CCO[C@@H](C)COc1nccc(-c2nn(C3CCCCO3)c3ccc(O[Si](C)(C)C(C)(C)C)cc23)n1]

To a solution of [3-[2-[(2S)-2-[(3R)-3-benzyloxybutoxy]propoxy]pyrimidin-4-yl]-1-tetrahydro pyran-2-yl-indazol-5-yl]oxy-tert-butyl-dimethyl-silane (650 mg, 1.006 mmol) in dichloro methane (20 mL) and pH 7 phosphate buffer (1 mL) at RT was added 2,3-dichloro-5,6-dicyano-p-benzoquinone (571 mg, 2.515 mmol). The reaction mixture was stirred at RT for 16 h. The reaction mixture was quenched with a saturated aqueous Na₂CO₃ solution and ethyl acetate was added. The phases were separated and the aqueous layer was extracted with ethyl acetate. The combined organic layers were washed with water and brine, dried over anhydrous sodium sulfate, filtered and evaporated under reduced pressure. The residue was purified by silica gel column chromatography using cyclohexane/ethyl acetate 100/0 to 50/50 as eluent to afford (2R)-4-[(1S)-2-[4-[5-[tert-butyl(dimethyl)silyl]oxy-1-tetrahydropyran-2-yl-indazol-3-yl]pyrimidin-2-yl]oxy-1-methyl-ethoxy]butan-2-ol as a yellow oil.

LCMS method F: [M+H]⁺=557.4, t_{R}=3.62 min

### Preparation of Intermediate 327: [(1R)-3-[(1S)-2-[4-[5-[tert-butyl(dimethyl)silyl]oxy-1-tetrahydropyran-2-yl-indazol-3-yl]pyrimidin-2-yl]oxy-1-methyl-ethoxy]-1-methyl-propyl]methanesulfonate

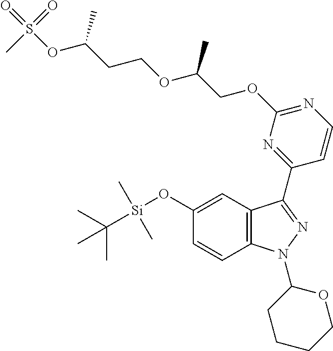 [SMILES: C[C@H](CCO[C@@H](C)COc1nccc(-c2nn(C3CCCCO3)c3ccc(O[Si](C)(C)C(C)(C)C)cc23)n1)OS(C)(=O)=O]

To a solution of (2R)-4-[(1S)-2-[4-[5-[tert-butyl(dimethyl)silyl]oxy-1-tetrahydropyran-2-yl-indazol-3-yl]pyrimidin-2-yl]oxy-1-methyl-ethoxy]butan-2-ol (370 mg, 0.665 mmol) and triethylamine (185 μL, 1.330 mmol) in dry dichloromethane (15 mL) at 0° C. was added methanesulfonyl chloride (77 μL, 0.998 mmol). The reaction mixture was stirred at RT for 4 h. Additional methanesulfonyl chloride (26 μL, 0.333 mmol) was added at RT. The reaction was stirred at RT for 16 h. Additional triethylamine (46 μL, 0.333 mmol) and methanesulfonyl chloride (26 μL, 0.333 mmol) were added at RT. The reaction was stirred at RT for 24 h. Additional triethylamine (556 μL, 3.990 mmol) and methanesulfonyl chloride (154 μL, 1.995 mmol) were added at RT. The reaction was stirred at RT for 2 h. The reaction mixture was quenched with brine and extracted with dichloromethane. The combined organic layers were dried over anhydrous sodium sulfate, filtered and evaporated under reduced pressure to afford [(1R)-3-[(1S)-2-[4-[5-[tert-butyl(dimethyl)silyl]oxy-1-tetrahydropyran-2-yl-indazol-3-yl] pyrimidin-2-yl]oxy-1-methyl-ethoxy]-1-methyl-propyl]methanesulfonate as an orange oil which was used in the next step without further purification.

LCMS method F: [M+H]⁺=635.4, t_{R}=3.70 min

### Preparation of Intermediate 328: (9S,13S)-9,13-dimethyl-19-(oxan-2-yl)-7,10,14-trioxa-5,19,20,23-tetraazatetracyclo[13.5.2.1^{2,6}.0^{18,21}]tricosa-1(20),2(23),3,5, 15(22),16,18(21)-heptaene

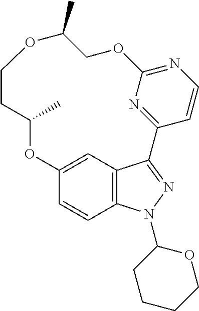 [SMILES: C[C@H]1COc2nccc(n2)-c2nn(C3CCCCO3)c3ccc(cc23)O[C@@H](C)CCO1]

To a solution of sodium hydride (60% dispersion in mineral oil) (78 mg, 1.941 mmol) in dry DMF (100 mL) at RT was added dropwise a solution of [(1R)-3-[(1S)-2-[4-[5-[tert-butyl (dimethyl)silyl]oxy-1-tetrahydropyran-2-yl-indazol-3-yl]pyrimidin-2-yl]oxy-1-methyl-ethoxy]-1-methyl-propyl] methanesulfonate (410 mg, 0.647 mmol) in dry DMF (51 mL). The reaction mixture was stirred at RT for 16 h. Additional sodium hydride (60% dispersion in mineral oil) (52 mg, 1.294 mmol) was added at RT and the reaction mixture was stirred at RT for 2 h. The reaction mixture was quenched with water and the solvent was removed under reduced pressure. The resulting solid was triturated with water, filtered and washed with water. The residue was purified by silica gel column chromatography using cyclohexane/ethyl acetate 100/0 to 70/30 as eluent to afford (9S,13S)-9,13-dimethyl-19-(oxan-2-yl)-7,10,14-trioxa-5,19, 20,23-tetraazatetracyclo[13.5.2.1^{2,6}.0^{18,21}]tricosa-1(20),2(23),3,5,15(22),16,18(21)-heptaene as a white solid.

LCMS method J: [M+H]⁺=425.5, t_{R}=4.41 min

### Preparation of Example 59: (9S,13S)-9,13-dimethyl-7,10,14-trioxa-5,19,20,23-tetraazatetracyclo[13.5.2.1^{2,6}.0^{18,21}]tricosa-1(20),2(23),3,5,15(22),16,18(21)-heptaene

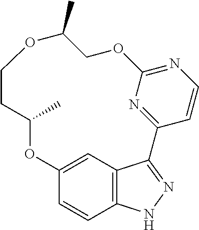 [SMILES: C[C@H]1COc2nccc(n2)-c2n[nH]c3ccc(cc23)O[C@@H](C)CCO1]

To a solution of (9S,13S)-9,13-dimethyl-19-(oxan-2-yl)-7,10,14-trioxa-5,19,20,23-tetraaza tetracyclo[13.5.2.1^{2,6}.0^{18,21}]tricosa-1(20),2(23),3,5,15(22),16,18(21)-heptaene (172 mg, 0.406 mmol) in methanol (30 mL) and water (4.3 mL) at RT was added p-toluenesulfonic acid monohydrate (386 mg, 2.030 mmol). The reaction mixture was stirred at 65° C. for 16 h. Additional p-toluenesulfonic acid monohydrate (154 mg, 0.812 mmol) was added and the reaction mixture was stirred at 70° C. for 16 h. Additional p-toluenesulfonic acid monohydrate (232 mg, 1.218 mmol) was added and the reaction mixture was stirred 80° C. for 16 h. The reaction mixture was concentrated under reduced pressure and a saturated aqueous Na₂CO₃ solution and ethyl acetate were added. The phases were separated and the aqueous layer was extracted with ethyl acetate. The combined organic layers were washed with water and brine, dried over anhydrous sodium sulfate, filtered and evaporated under reduced pressure. The residue was purified by silica gel column chromatography using cyclohexane/(ethyl acetate/ethanol (3-1)) 100/0 to 80/20 as eluent. The resulting product was triturated with acetonitrile, filtered and dried to afford (9S,13S)-9,13-dimethyl-7,10,14-trioxa-5,19,20,23-tetraazatetra cyclo[13.5.2.1^{2,6}.0^{18,21}] tricosa-1(20),2(23),3,5,15(22),16,18(21)-heptaene as a solid.

LCMS method J: [M+H]+=341.2, t_{R}=3.26 min

LCMS method N: [M+H]+=341.2, t_{R}=3.22 min

1H NMR (400 MHz, d6-DMSO) 13.53 (1H, s), 8.65 (1H, m), 8.58 (1H, d, J=5.1 Hz), 7.64 (1H, d, J=5.0 Hz), 7.52-7.49 (1H, m), 7.01-6.97 (1H, dd, J=2.4, 8.9 Hz), 5.52 (1H, d, J=12.3 Hz), 4.65-4.60 (1H, m), 4.20-4.15 (1H, dd, J=3.3, 12.5 Hz), 3.93-3.85 (2H, m), 3.24-3.17 (1H, m), 2.21-2.12 (1H, m), 1.48-1.40 (4H, m), 1.30 (3H, d, J=6.4 Hz) ppm.

### Example 60: (9R)-9-methyl-8,11,14-trioxa-4,5,19,20-tetraazatetracyclo[13.5.2.1^{2,5}.0^{18,21}] tricosa-1(20),2(23),3,15(22),16,18(21)-hexaene

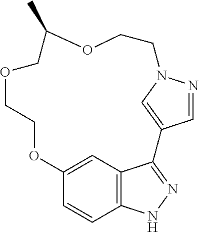 [SMILES: C[C@@H]1COCCOc2ccc3[nH]nc(c3c2)-c2cnn(c2)CCO1]

Example 60 is prepared according to the synthesis route described in general Scheme D.

### Preparation of Intermediate 329: ethyl (2R)-2-((tetrahydro-2H-pyran-2-yl)oxy) propanoate

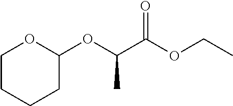

To a solution of ethyl (R)-2-hydroxypropanoate (25 g, 211.63 mmol) in dichloromethane (650 mL) was added 10-camphor sulfonic acid (4.91 g, 21.16 mmol) and 3,4-dihydro-2H-pyran (38.7 mL, 423.26 mmol) The reaction mixture was stirred at RT for 5 h. The reaction mixture was diluted with dichloromethane and washed with brine. The organic layer was dried over anhydrous magnesium sulfate, filtered and evaporated under reduced pressure. The residue was purified by silica gel column chromatography using heptane/ethyl acetate 100/0 to 80/10 as eluent to afford ethyl (2R)-2-((tetrahydro-2H-pyran-2-yl)oxy)propanoate as a colorless oil.

LCMS method B: [M+Na]⁺=225.0, t_{R}=1.103 min

### Preparation of Intermediate 330: ((2R)-2-((tetrahydro-2H-pyran-2-yl)oxy)propan-1-ol

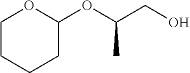

To a solution of ethyl (2R)-2-((tetrahydro-2H-pyran-2-yl)oxy)propanoate (31.2 g, 154.26 mmol) in ethanol (250 mL) and THF (250 mL) was added calcium chloride (17.12 g, 154.26 mmol) and sodium borohydride (11.67 g, 308.53 mmol). The reaction mixture was stirred at RT for 16 h. The reaction mixture was diluted with ethyl acetate, cooled at 0° C. and water was added. The suspension was filtered over a pad of Celite, washed with ethyl acetate and chloroform/isopropyl alcohol mixture. The combined organic layers were dried over anhydrous magnesium sulfate, filtered and evaporated under reduced pressure. The residue was purified by silica gel column chromatography using heptane/ethyl acetate 100/0 to 80/20 as eluent to afford ((2R)-2-((tetrahydro-2H-pyran-2-yl)oxy)propan-1-ol as a yellowish oil.

GCMS method A [M−H]⁻=not detected, t_{R}=6.4 min

### Preparation of Intermediate 331: 2-(((R)-1-(benzyloxy) propan-2-yl)oxy)tetrahydro-2H-pyran

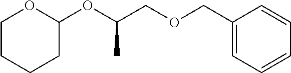 [SMILES: C[C@H](COCc1ccccc1)OC1CCCCO1]

To a solution of (2R)-2-((tetrahydro-2H-pyran-2-yl)oxy)propan-1-ol (15.9 g, 99.24 mmol) in dry DMF (225 mL) at 0° C. was added dropwise sodium hydride (60% dispersion in mineral oil) (5.95 g, 148.87 mmol). The reaction mixture was stirred at 0° C. for 1 h. A solution of benzyl bromide (23.57 mL, 198.49 mmol) was added dropwise and the reaction mixture was stirred at RT for 3 h. Water was added and the aqueous layer was extracted with ethyl acetate. The combined organic layers were washed with brine, dried over anhydrous magnesium sulfate, filtered and evaporated under reduced pressure. The residue was purified by silica gel column chromatography using heptane/ethyl acetate 100/0 to 80/20 as eluent to afford 2-(((R)-1-(benzyloxy)propan-2-yl)oxy)tetrahydro-2H-pyran as a colorless oil.

GCMS method A [M−H]⁻=248.9, t_{R}=9.57 min

### Preparation of Intermediate 332: (R)-1-(benzyloxy)propan-2-ol

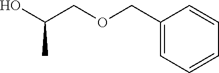

To a solution of 2-(((R)-1-(benzyloxy)propan-2-yl)oxy)tetrahydro-2H-pyran (20.3 g, 81.09 mmol) in methanol (245 mL) was added p-toluenesulfonic acid monohydrate (771 mg, 4.05 mmol) and the reaction mixture was stirred at RT for 63 h. The reaction mixture was cooled at 0° C. and a saturated aqueous NaHCO₃ solution was added. The solvent was evaporated reduced pressure and methanol was added and evaporated under reduced pressure. The residue was dissolved in diethyl ether and the organic layer was washed with brine. The organic layer was dried over anhydrous magnesium sulfate, filtered and evaporated under reduced pressure. The residue was purified by silica gel column chromatography using heptane/ethyl acetate 100/0 to 80/20 as eluent to afford (R)-1-(benzyloxy)propan-2-ol as a colorless oil.

LCMS method B: [M+H]⁺=167.1, t_{R}=0.537 min

### Preparation of Intermediate 333: 2-(2-(((R)-1-(benzyloxy)propan-2-yl)oxy)ethoxy) tetrahydro-2H-pyran

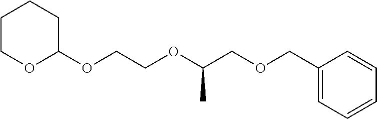 [SMILES: C[C@H](COCc1ccccc1)OCCOC1CCCCO1]

To a solution of (R)-1-(benzyloxy)propan-2-ol (8.6 g, 51.73 mmol) in THF (156 mL) was added potassium hydroxide (11.61 g, 206.95 mmol) and 2-(2-bromoethoxy)tetrahydro-2H-pyran (15.67 mL, 103.47 mmol). The reaction mixture was stirred at 80° C. for 15 h. The reaction mixture was diluted with dichloromethane and washed with brine. The organic layer was dried over anhydrous magnesium sulfate, filtered and evaporated under reduced pressure. The residue was purified by silica gel column chromatography using heptane/ethyl acetate 100/0 to 90/10 as eluent to afford 2-(2-(((R)-1-(benzyloxy)propan-2-yl)oxy)ethoxy)tetrahydro-2H-pyran as a colorless oil.

LCMS method B: [M+NH₄]⁺=312.1, t_{R}=0.979 min

### Preparation of Intermediate 334: (R)-2-((1-(benzyloxy)propan-2-yl)oxy)ethan-1-ol

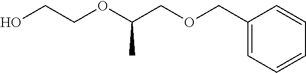

To a solution of 2-(2-(((R)-1-(benzyloxy)propan-2-yl)oxy)ethoxy)tetrahydro-2H-pyran (3.7 g, 12.56 mmol) in methanol (40 mL) was added p-toluenesulfonic acid monohydrate (239 mg, 1.25 mmol). The reaction mixture was stirred at 60° C. for 15 h. The solvent was evaporated under reduced pressure and the residue was diluted with dichloromethane. The organic layer was washed with a saturated aqueous NaHCO₃ solution and brine. The organic layer was dried over anhydrous magnesium sulfate, filtered and evaporated under reduced pressure. The residue was purified by silica gel column chromatography using heptane/ethyl acetate 100/0 to 50/50 as eluent to afford (R)-2-((1-(benzyloxy)propan-2-yl)oxy)ethan-1-ol as a colorless oil.

LCMS method B: [M+H]⁺=211.0, t_{R}=0.573 min

### Preparation of Intermediate 335: (R)-2-((1-(Benzyloxy)propan-2-yl)oxy)ethyl methanesulfonate

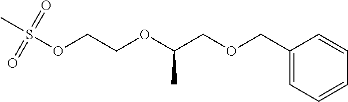 [SMILES: C[C@H](COCc1ccccc1)OCCOS(C)(=O)=O]

To a solution of (R)-2-((1-(benzyloxy)propan-2-yl)oxy)ethan-1-ol (2.015 g, 9.582 mmol) in dichloromethane (100 mL) at 0° C. under nitrogen atmosphere was added triethylamine (2 mL, 14.37 mmol) and methanesulfonyl chloride (964 μL, 12.45 mmol). The reaction mixture was stirred for 1 h from 0° C. to RT. The reaction mixture was diluted with dichloromethane and washed with a saturated aqueous NaHCO₃ solution. The combined organic layers were washed with brine, dried over anhydrous magnesium sulfate, filtered and evaporated under reduced pressure to afford (R)-2-((1-(Benzyloxy)propan-2-yl)oxy)ethyl methanesulfonate as a colorless oil which was used without further purification in the next step.

LCMS method B: [M+H]⁺=289.1, t_{R}=0.762 min

### Preparation of Intermediate 336: (R)-1-(2-((1-(benzyloxy)propan-2-yl)oxy)ethyl)-4-bromo-1H-pyrazole

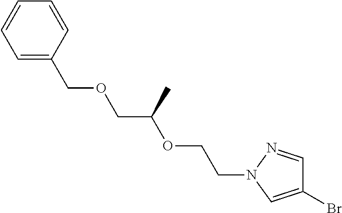 [SMILES: C[C@H](COCc1ccccc1)OCCn1cc(Br)cn1]

To a solution of (R)-2-((1-(benzyloxy)propan-2-yl)oxy)ethyl methanesulfonate (2.784 g, 9.582 mmol) in acetonitrile (50 mL) was added cesium carbonate (6.24 g, 19.16 mmol) and the reaction mixture was stirred at RT for 10 min. 4-Bromopyrazole (1.54 g, 10.54 mmol) was added and the reaction mixture was stirred at 90° C. for 15 h. The reaction mixture was diluted with ethyl acetate and washed with brine. The organic layer was dried over anhydrous magnesium sulfate, filtered and evaporated under reduced pressure. The residue was purified by silica gel column chromatography using heptane/ethyl acetate 100/0 to 70/30 as eluent to afford (R)-1-(2-((1-(benzyloxy)propan-2-yl)oxy)ethyl)-4-bromo-1H-pyrazole as a colorless oil.

LCMS method B: [M+H]⁺=339.0-340.9, t_{R}=0.990 min

### Preparation of Intermediate 337: (R)-2-(2-(4-bromo-1H-pyrazol-1-yl)ethoxy)propan-1-ol

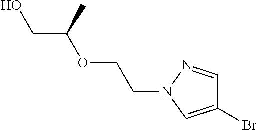

To a solution of (R)-1-(2-((1-(benzyloxy)propan-2-yl)oxy)ethyl)-4-bromo-1H-pyrazole (3 g, 8.84 mmol) in ethanol (62 mL) was added aqueous hydrochloric acid solution (44.0 mL) and the reaction mixture was stirred at 80° C. for 15 h. The reaction mixture was concentrated under reduced pressure and the residue was diluted with ethyl acetate and washed with a saturated aqueous NaHCO₃ solution and brine. The organic layer was dried over anhydrous magnesium sulfate, filtered and evaporated under reduced pressure. The residue was purified by silica gel column chromatography using heptane/ethyl acetate 100/0 to 80/20 as eluent to afford (R)-2-(2-(4-bromo-1H-pyrazol-1-yl)ethoxy)propan-1-ol as a colorless oil.

LCMS method B: [M+H]⁺=249.0-250.9, t_{R}=0.437 min

### Preparation of Intermediate 338: 1-[2-[(1R)-2-(2-benzyloxyethoxy)-1-methyl-ethoxy] ethyl]-4-bromo-pyrazole

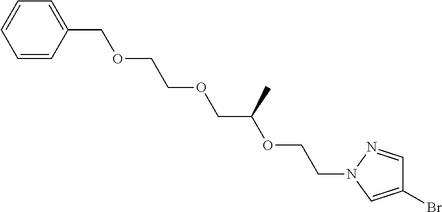 [SMILES: C[C@H](COCCOCc1ccccc1)OCCn1cc(Br)cn1]

To a solution of (R)-2-(2-(4-bromo-1H-pyrazol-1-yl)ethoxy)propan-1-ol (1.415 g, 5.68 mmol) in THF (20 ml) was added potassium hydroxide (1.275 g, 22.72 mmol) and benzyl 2-bromoethyl ether (1.797 mL, 11.36 mmol). The reaction mixture was stirred at 80° C. for 15 h. The reaction mixture was diluted with dichloromethane and washed with brine. The organic layer was dried over anhydrous magnesium sulfate, filtered and evaporated under reduced pressure. The residue was purified by silica gel column chromatography using heptane/ethyl acetate 100/0 to 80/20 as eluent to afford 1-[2-[(1R)-2-(2-benzyloxyethoxy)-1-methyl-ethoxy]ethyl]-4-bromo-pyrazole as a colorless oil.

LCMS method B: [M+H]⁺=383.0, t_{R}=0.995 min

### Preparation of Intermediate 339: [3-[1-[2-[(1R)-2-(2-benzyloxyethoxy)-1-methyl-ethoxy]ethyl]pyrazol-4-yl]-1-tetrahydropyran-2-yl-indazol-5-yl]oxy-tert-butyl-dimethyl-silane

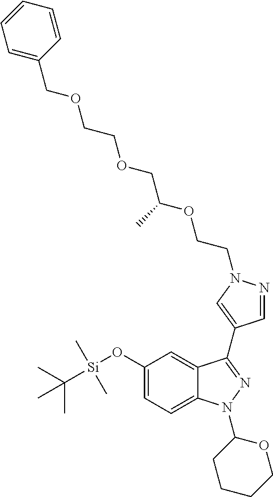 [SMILES: C[C@H](COCCOCc1ccccc1)OCCn1cc(-c2nn(C3CCCCO3)c3ccc(O[Si](C)(C)C(C)(C)C)cc23)cn1]

To a solution of tert-butyl-dimethyl-[1-tetrahydropyran-2-yl-3-(4,4,5,5-tetramethyl-1,3,2-dioxaborolan-2-yl)indazol-5-yl]oxy-silane intermediate 61 (1.3 g, 2.85 mmol) in dioxane (10 mL) and water (1 ml) at RT was added 1-[2-[(1R)-2-(2-benzyloxyethoxy)-1-methyl-ethoxy]ethyl]-4-bromo-pyrazole (990 mg, 2.59 mmol) and potassium phosphate tribasic (1.65 g, 7.77 mmol). The reaction mixture was purged with argon for 15 min then XPhos (123 mg, 0.26 mmol) and tetrakis(triphenylphosphine)palladium(0) (90 mg, 0.08 mmol) were added. The reaction mixture was stirred at 80° C. for 45 min under microwave irradiations. The solvent was evaporated under reduced pressure. The residue was partitioned between ethyl acetate and water. The phases were separated and the aqueous layer was extracted with ethyl acetate. The organic layer was washed with brine, dried over anhydrous sodium sulfate, filtered and evaporated under reduced pressure. The residue was purified by silica gel column chromatography using cyclohexane/ethyl acetate 50/50 as eluent to afford [3-[1-[2-[(1R)-2-(2-benzyloxyethoxy)-1-methyl-ethoxy]ethyl]pyrazol-4-yl]-1-tetrahydropyran-2-yl-indazol-5-yl]oxy-tert-butyl-dimethyl-silane as a brown oil.

LCMS method F: [M+H]⁺=635.6, t_{R}=3.75 min

### Preparation of Intermediate 340: 2-[(2R)-2-[2-[4-[5-[tert-butyl(dimethyl)silyl]oxy-1-tetrahydropyran-2-yl-indazol-3-yl]pyrazol-1-yl]ethoxy]propoxy]ethanol

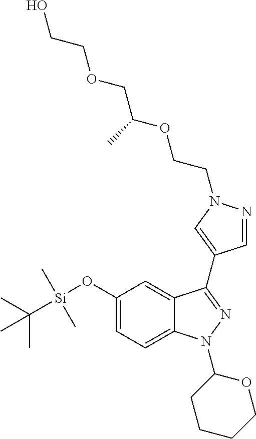 [SMILES: C[C@H](COCCO)OCCn1cc(-c2nn(C3CCCCO3)c3ccc(O[Si](C)(C)C(C)(C)C)cc23)cn1]

To a solution of [3-[1-[2-[(1R)-2-(2-benzyloxyethoxy)-1-methyl-ethoxy]ethyl]pyrazol-4-yl]-1-tetrahydropyran-2-yl-indazol-5-yl]oxy-tert-butyl-dimethyl-silane (790 mg, 1.25 mmol) in ethyl acetate (10 ml) under N₂ was added palladium on carbon (13 mg, 0.12 mmol). The reaction mixture was stirred at 60° C. for 24 h under hydrogen atmosphere. The reaction mixture was filtered over a pad of Celite and the filtrate was evaporated reduced pressure to afford 2-[(2R)-2-[2-[4-[5-[tert-butyl(dimethyl)silyl]oxy-1-tetrahydropyran-2-yl-indazol-3-yl]pyrazol-1-yl]ethoxy]propoxy]ethanol as a colorless oil.

LCMS method F: [M+H]⁺=545.5, t_{R}=3.21 min

### Preparation of Intermediate 341: 2-[(2R)-2-[2-[4-[5-[tert-butyl(dimethyl)silyl]oxy-1-tetrahydropyran-2-yl-indazol-3-yl]pyrazol-1-yl]ethoxy]propoxy]ethylmethane sulfonate

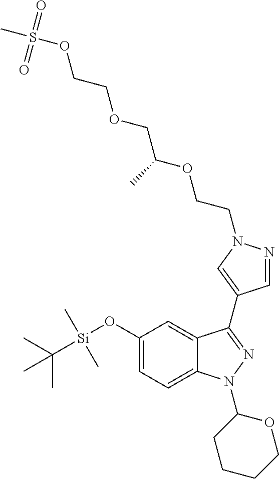 [SMILES: C[C@H](COCCOS(C)(=O)=O)OCCn1cc(-c2nn(C3CCCCO3)c3ccc(O[Si](C)(C)C(C)(C)C)cc23)cn1]

To a solution of 2-[(2R)-2-[2-[4-[5-[tert-butyl(dimethyl)silyl]oxy-1-tetrahydropyran-2-yl-indazol-3-yl]pyrazol-1-yl]ethoxy]propoxy]ethanol (626 mg, 1.15 mmol) and triethylamine (238 μL, 1.72 mmol) in dichloromethane (10 mL) at 0° C. was added methanesulfonyl chloride (97 μL, 1.27 mmol). The reaction mixture was stirred at 0° C. for 5 min then allowed to warm up to RT and stirred for 4 h. Additional triethylamine (238 μL, 1.72 mmol) and methanesulfonyl chloride (97 μL, 1.27 mmol) were added at 0° C. The reaction mixture was stirred at 0° C. for 5 min then allowed to warm up to RT and stirred for 12 h. A saturated aqueous ammonium chloride solution was added. The phases were separated and the aqueous layer was extracted with dichloromethane. The combined organic layers were washed with a saturated aqueous NaHCO₃ solution and brine, dried over anhydrous anhydrous sodium sulfate, filtered and evaporated under reduced pressure to afford 2-[(2R)-2-[2-[4-[5-[tert-butyl(dimethyl)silyl]oxy-1-tetrahydropyran-2-yl-indazol-3-yl]pyrazol-1-yl]ethoxy]propoxy]ethyl methanesulfonate as a colorless oil which was used in the next step without further purification.

LCMS method F: [M+H]⁺=623.5, t_{R}=3.36 min

### Preparation of Intermediate 342: (9R)-9-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene

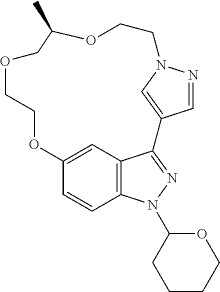 [SMILES: C[C@@H]1COCCOc2ccc3c(c2)c(nn3C2CCCCO2)-c2cnn(c2)CCO1]

To a suspension of cesium carbonate (839 mg, 2.57 mmol) in dry DMF (200 mL) at 80° C. was added dropwise 2-[(2R)-2-[2-[4-[5-[tert-butyl(dimethyl)silyl]oxy-1-tetrahydropyran-2-yl-indazol-3-yl]pyrazol-1-yl]ethoxy]propoxy]ethyl methanesulfonate (400 mg, 0.64 mmol) in dry DMF (200 mL). The reaction mixture was stirred at 80° C. for 1 h. The reaction mixture was filtered on a pad of Celite and washed with ethyl acetate. The filtrate was concentrated under reduced pressure. The residue was purified by silica gel column chromatography using cyclohexane/ethyl acetate 100/0 to 60/40 as eluent to afford (9R)-9-methyl-19-(oxan-2-yl)-8,11,14-trioxa-4,5,19,20-tetraazatetracyclo[13.5.2.1^{2,5}.0^{18,21}]tricosa-1(20),2(23),3,15(22), 16,18(21)-hexaene as a yellow oil.

LCMS method F: [M+H]⁺=413.4, t_{R}=2.50 min

### Preparation of Example 60: (9R)-9-methyl-8,11,14-trioxa-4,5,19,20-tetraazatetra cyclo[13.5.2.1^{2,5}.0^{18,21}]tricosa-1(20),2(23),3,15(22),16,18(21)-hexaene

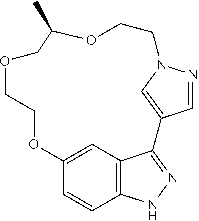 [SMILES: C[C@@H]1COCCOc2ccc3[nH]nc(c3c2)-c2cnn(c2)CCO1]

To a solution of (9R)-9-methyl-19-(oxan-2-yl)-8,11,14-trioxa-4,5,19,20-tetraazatetracyclo [13.5.2.1^{2,5}.0^{18,21}]tricosa-1(20),2(23),3,15(22),16,18(21)-hexaene (170 mg, 0.41 mmol) in methanol (4 mL) and water (0.6 mL) was added p-toluenesulfonic acid monohydrate (392 mg, 2.06 mmol). The reaction mixture was stirred at 80° C. for 6 h. The solvent was evaporated under reduced pressure. The residue was partitioned between dichloromethane and a saturated aqueous NaHCO₃ solution. The phases were separated and the aqueous layer was extracted with dichloromethane. The combined organic layers were washed with brine, dried over anhydrous sodium sulfate, filtered and evaporated under reduced pressure. The resulting oil was triturated with diethyl ether, filtered and dried to afford (9R)-9-methyl-8,11,14-trioxa-4,5,19,20-tetraazatetracyclo[13.5.2.1^{2,5}.0^{18,21}]tricosa-1(20),2(23),3,15(22),16,18(21)-hexaene as a solid.

LCMS method F: [M+H]⁺=329.3, t_{R}=1.93 min

LCMS method G: [M+H]⁺=329.3, t_{R}=1.92 min

1H NMR (400 MHz, CDCl₃) 8.62 (1H, s), 8.07-8.02 (2H, m), 7.37-7.33 (1H, m), 7.09 (1H, dd, J=2.3, 8.9 Hz), 4.52-4.43 (4H, m), 4.06-4.00 (1H, m), 3.91-3.71 (4H, m), 3.60-3.56 (2H, m), 1.14 (3H, d, J=6.3 Hz) ppm.

### Example 61: (9S)-9-methyl-8,11,14-trioxa-4,5,19,20-tetraazatetracyclo[13.5.2.1^{2,5}.0^{18,21}] tricosa-1(20),2(23),3,15(22),16,18(21)-hexaene

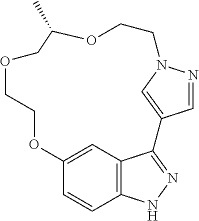 [SMILES: C[C@H]1COCCOc2ccc3[nH]nc(c3c2)-c2cnn(c2)CCO1]

Example 61 is prepared according to the synthesis route described in general Scheme D, following the same synthesis procedures as for Example 60.

### Preparation of Intermediate 343: (9S)-9-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene

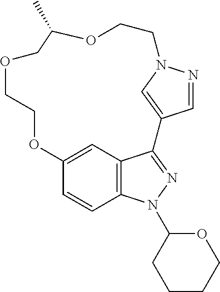 [SMILES: C[C@H]1COCCOc2ccc3c(c2)c(nn3C2CCCCO2)-c2cnn(c2)CCO1]

To a suspension of cesium carbonate (780 mg, 2.4 mmol) in anhydrous DMF (255 mL) at 80° C. was added dropwise 2-[(2S)-2-[2-[4-[5-[tert-butyl(dimethyl)silyl]oxy-1-tetrahydropyran-2-yl-indazol-3-yl]pyrazol-1-yl]ethoxy]propoxy]ethylmethanesulfonate (500 mg, 0.76 mmol) in DMF (255 mL). The reaction mixture was stirred at 80° C. for 1 h. The reaction mixture was filtered and washed with ethyl acetate The residue was diluted with water. The phases were separated and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent to afford (9S)-9-methyl-19-(oxan-2-yl)-8,11,14-trioxa-4,5,19,20-tetraazatetracyclo[13.5.2.1^{2,5}.0^{18,21}]tricosa-1(20),2(23),3,15(22), 16,18(21)-hexaene as a colorless oil.

LCMS method F: [M+H]⁺=413.3, t_{R}=2.56 min

### Preparation of Example 61: (9S)-9-methyl-8,11,14-trioxa-4,5,19,20-tetraazatetra cyclo[13.5.2.1^{2,5}.0^{18,21}]tricosa-1(20),2(23),3,15(22),16,18(21)-hexaene

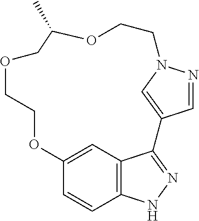 [SMILES: C[C@H]1COCCOc2ccc3[nH]nc(c3c2)-c2cnn(c2)CCO1]

To a solution of (9S)-9-methyl-19-(oxan-2-yl)-8,11,14-trioxa-4,5,19,20-tetraazatetracyclo [13.5.2.1^{2,5}.0^{18,21}]tricosa-1(20),2(23),3,15(22),16,18(21)-hexaene (144 mg, 0.35 mmol) in methanol (6 mL) and water (1 mL) was added p-toluenesulfonic acid monohydrate (334 mg, 1.76 mmol). The reaction mixture was stirred at 65° C. overnight. The reaction mixture was diluted with dichloromethane and saturated aqueous NaHCO₃ solution. The aqueous layer was extracted with dichloromethane. The combined organic layers were washed brine, dried over anhydrous sodium sulfate, filtered and evaporated under reduced pressure. The resulting solid was triturated with acetonitrile, filtered and dried to afford (9S)-9-methyl-8,11,14-trioxa-4,5,19,20-tetraazatetracyclo[13.5.2.1^{2,5}.0^{18,21}]tricosa-1(20),2(23),3,15(22),16,18(21)-hexaene as a solid.

LCMS method F: [M+H]⁺=329.2, t_{R}=1.97 min

LCMS method G: [M+H]⁺=329.2, t_{R}=1.90 min 1H NMR (400 MHz, d6-DMSO) 12.73 (1H, s), 8.53 (1H, s), 7.88 (1H, d, J=1.7 Hz), 7.81 (1H, s), 7.39 (1H, d, J=8.8 Hz), 6.99 (1H, dd, J=2.4, 8.8 Hz), 4.36 (4H, m), 3.93 (1H, m), 3.79 (3H, m), 3.67 (1H, m), 3.53 (2H, m), 1.07 (3H, d, J=6.3 Hz) ppm.

### Example 62: (13S)-13-methyl-7,10,14-trioxa-4-thia-19,20,23-triazatetracyclo[13.5.2.1^{2,5}.0^{18,21}]tricosa-1(20),2,5(23),15(22),16,18(21)-hexaene

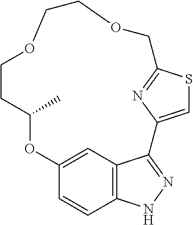 [SMILES: C[C@H]1CCOCCOCc2nc(cs2)-c2n[nH]c3ccc(cc23)O1]

Example 62 is prepared according to the synthesis route described in general Scheme D.

### Preparation of Intermediate 344: 2-[2-[(3R)-3-benzyloxybutoxy]ethoxy]tetra hydropyran

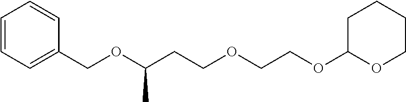 [SMILES: C[C@H](CCOCCOC1CCCCO1)OCc1ccccc1]

To a suspension of sodium hydride (60% dispersion in mineral oil) (1.087 g, 29.9 mmol) in DMF (45 mL) at 0° C. was added dropwise a solution of 2-tetrahydropyran-2-yloxyethanol (3.97 g, 27.18 mmol) in DMF (50 mL). The reaction mixture was stirred at 0° C. for 15 min. A solution of [(3R)-3-benzyloxybutyl]-4-methylbenzenesulfonate (intermediate 129) (10 g, 29.9 mmol) in DMF (50 mL) was added dropwise and the reaction mixture was stirred at 70° C. for 2 h. The reaction mixture was cooled to RT, quenched with water and poured into ethyl acetate. The phases were separated and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate using 100/0 to 70/30 as eluent to afford 2-[2-[(3R)-3-benzyloxybutoxy]ethoxy]tetrahydropyran as a colorless oil.

LCMS method F: [M+Na]⁺=331.2, t_{R}=2.87 min

### Preparation of Intermediate 345: 2-[(3R)-3-benzyloxybutoxy] ethanol

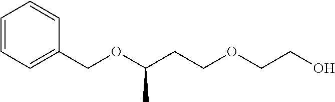 [SMILES: C[C@H](CCOCCO)OCc1ccccc1]

To a solution of 2-[2-[(3R)-3-benzyloxybutoxy]ethoxy]tetrahydropyran (3.51 g, 12.52 mmol) in methanol (100 mL) and water (20 mL) was added p-toluenesulfonic acid monohydrate (4.76 g, 25.05 mmol). The reaction mixture was stirred at 50° C. for 16 h. The solvent was removed under reduced pressure and the residue was diluted with ethyl acetate. The phases were separated and the aqueous layer was extracted with ethyl acetate. The combined organic layers were washed with a saturated aqueous NaHCO₃ solution and brine, dried over anhydrous sodium sulfate, filtered and evaporated under reduced pressure to afford 2-[(3R)-3-benzyloxybutoxy] ethanol as a colorless oil which was used in the next step without further purification.

LCMS method F: [M+Na]⁺=247.3, t_{R}=1.98 min

### Preparation of Intermediate 346: 2-[(3R)-3-benzyloxybutoxy]ethyl 4-methylbenzene sulfonate

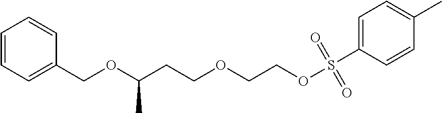 [SMILES: Cc1ccc(S(=O)(=O)OCCOCC[C@@H](C)OCc2ccccc2)cc1]

To a solution of 2-[(3R)-3-benzyloxybutoxy]ethanol (600 mg, 2.67 mmol) and triethylamine (746 μL, 5.35 mmol) in dichloromethane (15 mL) at 0° C. was added portionwise p-toluenesulfonyl chloride (663 mg, 3.48 mmol). The reaction mixture was stirred at RT overnight. The residue was diluted with saturated NaHCO₃ solution and extracted with dichloromethane. The combined organic layers were washed with brine, dried over anhydrous sodium sulfate, filtered and evaporated under reduced pressure to afford 2-[(3R)-3-benzyloxybutoxy]ethyl 4-methylbenzenesulfonate as a yellow oil which was used in the next step without further purification.

LCMS method F: [M+Na]⁺=401.3, t_{R}=2.52 min

### Preparation of Intermediate 347: (4-bromothiazol-2-yl)methanol

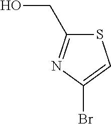

To a solution of 4-bromothiazole-2-carbaldehyde (2.88 g, 15 mmol) in methanol (30 mL) at 0° C. was added portionwise sodium borohydride (570 mg, 15 mmol). The reaction mixture was stirred at RT for 2 h. The reaction mixture was diluted with ethyl acetate and washed with water and brine. The organic layer was dried over anhydrous sodium sulfate, filtered and evaporated under reduced pressure. The residue was purified by silica gel column chromatography using cyclohexane/ethyl acetate 100/0 to 50/50 as eluent to afford (4-bromothiazol-2-yl)methanol as a yellow oil.

LCMS method F: [M+H]⁺=194.1-196.1, t_{R}=1.25 min

### Preparation of Intermediate 348: 2-[2-[(3R)-3-benzyloxybutoxy]ethoxymethyl]-4-bromo-thiazole

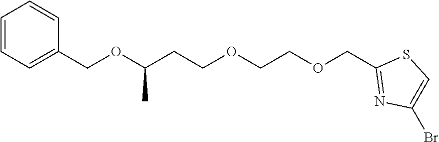 [SMILES: C[C@H](CCOCCOCc1nc(Br)cs1)OCc1ccccc1]

To a solution of (4-bromothiazol-2-yl)methanol (470 mg, 2.42 mmol) in anhydrous acetonitrile (15 mL) at RT was added cesium carbonate (3.947 g, 12.11 mmol) and 2-[(3R)-3-benzyloxy butoxy]ethyl 4-methylbenzenesulfonate (intermediate 346) (1.009 g, 2.66 mmol). The reaction mixture was stirred at 70° C. in a sealed tube for 20 h. The reaction mixture was concentrated under reduced pressure, diluted with brine and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 80/20 as eluent. to afford 2-[2-[(3R)-3-benzyloxybutoxy]ethoxymethyl]-4-bromo-thiazole as a colorless oil.

LCMS method F: [M+H]⁺=400.1-402.1, t_{R}=2.98 min

### Preparation of Intermediate 349: [3-[2-[2-[(3R)-3-benzyloxybutoxy]ethoxymethyl] thiazol-4-yl]-1-tetrahydropyran-2-yl-indazol-5-yl]oxy-tert-butyl-dimethyl-silane

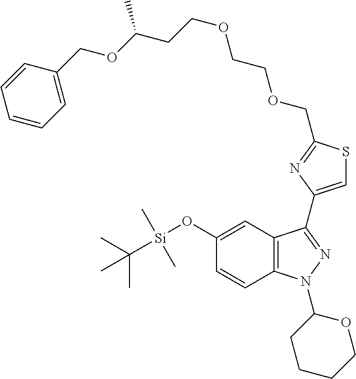 [SMILES: C[C@H](CCOCCOCc1nc(-c2nn(C3CCCCO3)c3ccc(O[Si](C)(C)C(C)(C)C)cc23)cs1)OCc1ccccc1]

To a solution of tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (852 mg, 1.86 mmol) in dioxane (12 mL) and water (1.2 mL) at RT was added 2-[2-[(3R)-3-benzyloxybutoxy]ethoxymethyl]-4-bromo-thiazole (620 mg, 1.55 mmol), potassium phosphate tribasic (986 mg, 4.65 mmol), XPhos (74 mg, 0.15 mmol) and tetrakis(triphenylphosphine)palladium(0) (90 mg, 0.08 mmol). The reaction mixture was stirred under microwave irradiations at 90° C. for 1.5 h. The reaction mixture was diluted with brine and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 80/20 as eluent. to afford [3-[2-[2-[(3R)-3-benzyloxybutoxy]ethoxymethyl]thiazol-4-yl]-1-tetrahydropyran-2-yl-indazol-5-yl]oxy-tert-butyl-dimethyl-silane as a brown oil.

LCMS method I: [M+H]⁺=652.5, t_{R}=3.70 min

### Preparation of Intermediate 350: (2R)-4-[2-[[4-[5-[tert-butyl(dimethyl)silyl]oxy-1-tetrahydropyran-2-yl-indazol-3-yl]thiazol-2-yl]methoxy]ethoxy]butan-2-ol

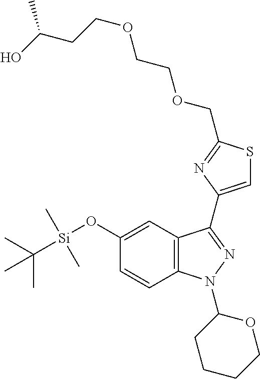 [SMILES: C[C@@H](O)CCOCCOCc1nc(-c2nn(C3CCCCO3)c3ccc(O[Si](C)(C)C(C)(C)C)cc23)cs1]

To a solution of [3-[2-[2-[(3R)-3-benzyloxybutoxy]ethoxymethyl]thiazol-4-yl]-1-tetrahydro pyran-2-yl-indazol-5-yl]oxy-tert-butyl-dimethyl-silane (955 mg, 1.46 mmol) in dichloromethane (29 mL) and pH 7 phosphate buffer (1.49 mL) at RT was added 2,3-dichloro-5,6-dicyano-p-benzoquinone (831 mg, 3.66 mmol). The reaction mixture was stirred at RT for 6 h. The reaction mixture was diluted with a saturated aqueous NaHCO₃ solution and extracted with ethyl acetate. The combined organic layers were washed with brine, dried over anhydrous sodium sulfate, filtered and evaporated under reduced pressure. The residue was purified by silica gel column chromatography using dichloromethane/ethyl acetate 100/0 to 60/40 as eluent to afford (2R)-4-[2-[[4-[5-[tert-butyl(dimethyl)silyl]oxy-1-tetrahydropyran-2-yl-indazol-3-yl]thiazol-2-yl]methoxy]ethoxy]butan-2-ol as a colorless oil.

LCMS method F: [M+H]⁺=562.4, t_{R}=3.53 min

### Preparation of Intermediate 351: [(1R)-3-[2-[[4-[5-[tert-butyl(dimethyl)silyl]oxy-1-tetrahydropyran-2-yl-indazol-3-yl]thiazol-2-yl]methoxy]ethoxy]-1-methyl-propyl] methanesulfonate

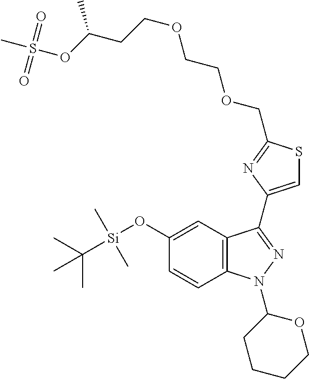 [SMILES: C[C@H](CCOCCOCc1nc(-c2nn(C3CCCCO3)c3ccc(O[Si](C)(C)C(C)(C)C)cc23)cs1)OS(C)(=O)=O]

To a solution of (2R)-4-[2-[[4-[5-[tert-butyl(dimethyl)silyl]oxy-1-tetrahydropyran-2-yl-indazol-3-yl]thiazol-2-yl]methoxy]ethoxy]butan-2-ol (460 g, 0.82 mmol) and triethylamine (228 μL, 1.64 mmol) in dichloromethane (8 mL) at 0° C. was added methanesulfonyl chloride (95 μL, 1.23 mmol). The reaction mixture was stirred at RT overnight. The reaction mixture was diluted with brine and extracted with dichloromethane. The combined organic layers were dried over anhydrous sodium sulfate, filtered and evaporated under reduced pressure to afford [(1R)-3-[2-[[4-[5-[tert-butyl(dimethyl)silyl]oxy-1-tetrahydropyran-2-yl-indazol-3-yl]thiazol-2-yl]methoxy]ethoxy]-1-methyl-propyl] methanesulfonate as a yellow oil which was used in the next step without further purification.

LCMS method I: [M+H]⁺=640.5, t_{R}=3.13 min

### Preparation of Intermediate 352: (13S)-13-methyl-19-(oxan-2-yl)-7,10,14-trioxa-4-thia-19,20,23-triazatetracyclo[13.5.2.1^{2,5}.0^{18,21}]tricosa-1(20),2,5(23),15(22),16, 18(21)-hexaene

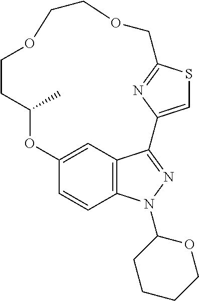 [SMILES: C[C@H]1CCOCCOCc2nc(cs2)-c2nn(C3CCCCO3)c3ccc(cc23)O1]

To a suspension of cesium carbonate (1.018 g, 3.13 mmol) in anhydrous DMF (195 mL) at 80° C. was added dropwise [(1R)-3-[2-[[4-[5-[tert-butyl(dimethyl)silyl]oxy-1-tetrahydropyran-2-yl-indazol-3-yl]thiazol-2-yl]methoxy]ethoxy]-1-methyl-propyl] methanesulfonate (500 mg, 0.78 mmol) in DMF (195 mL). The reaction mixture was stirred at 80° C. for 3 h. The reaction mixture was concentrated under reduced pressure, diluted with brine and extracted with ethyl acetate. The combined organic layers were dried over anhydrous sodium sulfate, filtered and evaporated under reduced pressure. The residue was purified by silica column chromatography using cyclohexane/(ethyl acetate/ethanol (3/1)) 100/0 to 90/10 as eluent to afford (13S)-13-methyl-19-(oxan-2-yl)-7,10,14-trioxa-4-thia-19,20,23-triazatetracyclo[13.5.2.1^{2,5}.0^{18,21}] tricosa-1(20),2,5(23),15(22),16,18(21)-hexaene as a colorless oil.

LCMS method F: [M+H]⁺=430.4, t_{R}=2.95 min

### Preparation of Example 62: (13S)-13-methyl-7,10,14-trioxa-4-thia-19,20,23-triazatetracyclo[13.5.2.1^{2,5}.0^{18,21}]tricosa-1 (20),2,5(23),15(22),16,18(21)-hexaene

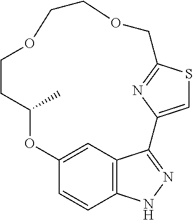 [SMILES: C[C@H]1CCOCCOCc2nc(cs2)-c2n[nH]c3ccc(cc23)O1]

To a solution of (13S)-13-methyl-19-(oxan-2-yl)-7,10,14-trioxa-4-thia-19,20,23-triazatetracyclo [13.5.2.1^{2,5}.0^{18,21}]tricosa-1(20),2,5(23),15(22),16,18(21)-hexaene (250 mg, 0.58 mmol) in methanol (7 mL) and water (1 mL) was added p-toluenesulfonic acid monohydrate (554 mg, 2.91 mmol). The reaction mixture was stirred at 65° C. overnight. The reaction mixture was concentrated under reduced pressure and the residue was neutralized by addition of a saturated aqueous NaHCO₃ solution. The mixture was diluted with ethyl acetate. The phases were evaporated and the aqueous phase was extracted with ethyl acetate. The combined organic layers were washed with brine, dried over sodium sulfate, filtered and evaporated under reduced pressure. The resulting product was recrystallized from acetonitrile, filtered and dried to afford (13S)-13-methyl-7,10,14-trioxa-4-thia-19,20,23-triazatetra cyclo[13.5.2.1^{2,5}.0^{18,21}]tricosa-1(20),2,5(23),15(22),16,18(21)-hexaene as a solid.

LCMS method F: [M+H]⁺=346.3, t_{R}=2.24 min

LCMS method G: [M+H]⁺=346.3, t_{R}=2.23 min 1H NMR (400 MHz, d6-DMSO) 13.03 (1H, s), 8.06 (1H, d, J=2.3 Hz), 7.92 (1H, s), 7.44-7.41 (1H, m), 6.95 (1H, dd, J=2.3, 8.9 Hz), 4.99-4.87 (2H, m), 4.67-4.61 (1H, m), 3.86-3.71 (2H, m), 3.69-3.55 (3H, m), 3.53-3.45 (1H, m), 2.16-2.08 (1H, m), 1.56-1.48 (1H, m), 1.37-1.34 (3H, m) ppm.

### Example 63: 8,11,14-trioxa-4,5,19,20-tetraazatetracyclo[13.5.2.1^{2,5}.0^{18,21}]tricosa-1(20), 2(23),3,15(22),16,18(21)-hexaene

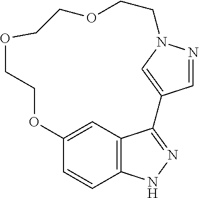 [SMILES: c1cc2[nH]nc3c2cc1OCCOCCOCCn1cc-3cn1]

Example 63 is prepared according to the synthesis route described in general Scheme B.

### Preparation of Intermediate 353: 1-[2-(2-benzyloxyethoxy)ethyl]-4-(4,4,5,5-tetramethyl-1,3,2-dioxaborolan-2-yl)pyrazole

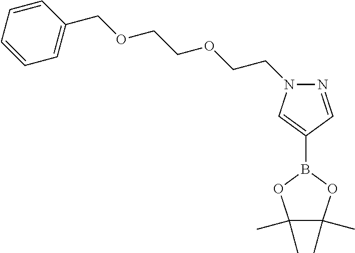 [SMILES: CC1(C)OB(c2cnn(CCOCCOCc3ccccc3)c2)OC1(C)C]

To a solution of 4-(4,4,5,5-tetramethyl-1,3,2-dioxaborolan-2-yl)-1H-pyrazole (986 mg, 5.08 mmol) in anhydrous DMF (20 mL) at RT was added potassium carbonate (1.05 g, 7.62 mmol) and 2-(2-benzyloxyethoxy)ethyl methanesulfonate (1.53 g, 5.58 mmol). The reaction mixture was stirred at 80° C. overnight. The reaction mixture was filtered and the filtrate was concentrated under reduced pressure. The residue was diluted with brine and extracted with ethyl acetate. The combined organic layers were dried over anhydrous sodium sulfate, filtered and evaporated under reduced to afford 1-[2-(2-benzyloxyethoxy)ethyl]-4-(4,4,5,5-tetramethyl-1,3,2-dioxaborolan-2-yl)pyrazole as a colorless oil.

LCMS method F: [M+H]⁺=373.3, t_{R}=2.69 min

### Preparation of Intermediate 354: 2-[2-[4-(4,4,5,5-tetramethyl-1,3,2-dioxaborolan-2-yl)pyrazol-1-yl]ethoxy]ethanol

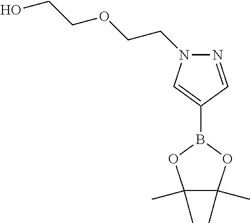 [SMILES: CC1(C)OB(c2cnn(CCOCCO)c2)OC1(C)C]

To a solution of 1-[2-(2-benzyloxyethoxy)ethyl]-4-(4,4,5,5-tetramethyl-1,3,2-dioxaborolan-2-yl)pyrazole (1.48 g, 3.98 mmol) in ethanol (40 ml) under N₂ was added palladium hydroxide (56 mg, 0.398 mmol). The reaction mixture was stirred at 60° C. for 24 h under hydrogen atmosphere. The reaction mixture was filtered over a pad of Celite and the filtrate was evaporated under reduced pressure to afford 2-[2-[4-(4,4,5,5-tetramethyl-1,3,2-dioxaborolan-2-yl)pyrazol-1-yl]ethoxy]ethanol as a colorless oil.

LCMS method F: [M+H]⁺=283.2, t_{R}=1.84 min

### Preparation of Intermediate 355: 1-[2-[2-(2-benzyloxyethoxy)ethoxy]ethyl]-4-(4,4,5,5-tetramethyl-1,3,2-dioxaborolan-2-yl)pyrazole

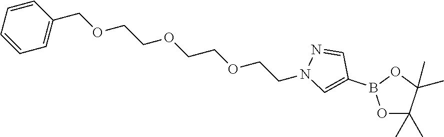 [SMILES: CC1(C)OB(c2cnn(CCOCCOCCOCc3ccccc3)c2)OC1(C)C]

To a solution of 2-[2-[4-(4,4,5,5-tetramethyl-1,3,2-dioxaborolan-2-yl)pyrazol-1-yl]ethoxy] ethanol (250 mg, 0.88 mmol) in DMF (5 mL) was added portionwise sodium hydride (60% dispersion in mineral oil) (88 mg, 1.32 mmol). The reaction mixture was stirred at RT for 30 min and 2-bromoethoxymethylbenzene (208 mg, 0.97 mmol) was added. The reaction mixture was stirred at 55° C. for 6 h and the solvent was evaporated under reduced pressure, diluted with water and extracted with ethyl acetate. The combined organic layers were washed with water, dried over anhydrous magnesium sulfate, filtered and evaporated under reduced pressure. The residue was purified by silica gel column chromatography using cyclohexane/ethyl acetate 100/0 to 70/30 as eluent to afford 1-[2-[2-(2-benzyloxyethoxy)ethoxy]ethyl]-4-(4,4,5,5-tetramethyl-1,3,2-dioxaborolan-2-yl)pyrazole as a colorless oil.

LCMS method F: [M+H]⁺=417.3, t_{R}=2.68 min

### Preparation of Intermediate 356: [3-[1-[2-[2-(2-benzyloxyethoxy)ethoxy]ethyl] pyrazol-4-yl]-1-tetrahydropyran-2-yl-indazol-5-yl]oxy-tert-butyl-dimethyl-silane

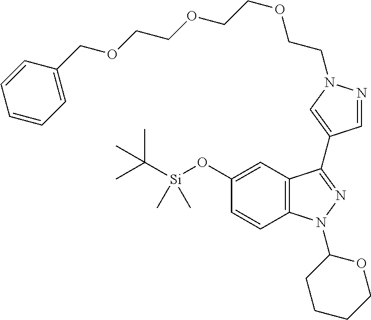 [SMILES: CC(C)(C)[Si](C)(C)Oc1ccc2c(c1)c(-c1cnn(CCOCCOCCOCc3ccccc3)c1)nn2C1CCCCO1]

To a degassed solution of 1-[2-[2-(2-benzyloxyethoxy)ethoxy]ethyl]-4-(4,4,5,5-tetramethyl-1,3,2-dioxaborolan-2-yl)pyrazole (178 mg, 0.42 mmol), 5-((tert-butyldimethylsilyl)oxy)-3-iodo-1-(tetrahydro-2H-pyran-2-yl)-1H-indazole (intermediate 15) (233 mg, 0.51 mmol) and potassium phosphate tribasic (267 mg, 1.26 mmol) in dioxane (6 mL) and water (0.3 mL) was added tetrakis(triphenylphosphine)palladium(0) (24 mg, 0.021 mmol) and 2-dicyclohexylphosphino-2′,4′,6′-triisopropylbiphenyl (20 mg, 0.042 mmol). The reaction mixture was stirred at 100° C. for 3 h. The reaction mixture was filtered through a pad of Celite and washed with ethyl acetate. The filtrate was diluted with water and extracted with ethyl acetate. The combined organic layers were washed with water, brine, dried over anhydrous magnesium sulfate, filtered and evaporated under reduced pressure. The residue was purified by silica gel column chromatography using cyclohexane/ethyl acetate 99/1 to 40/60 as eluent to afford [3-[1-[2-[2-(2-benzyloxyethoxy)ethoxy]ethyl]pyrazol-4-yl]-1-tetrahydropyran-2-yl-indazol-5-yl]oxy-tert-butyl-dimethyl-silane as a colorless oil.

LCMS method F: [M+H]⁺=621.4, t_{R}=3.63 min

### Preparation of Intermediate 357: 2-[2-[2-[4-[5-[tert-butyl(dimethyl)silyl]oxy-1-tetrahydropyran-2-yl-indazol-3-yl]pyrazol-1-yl]ethoxy]ethoxy]ethanol

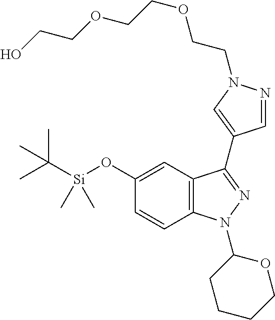 [SMILES: CC(C)(C)[Si](C)(C)Oc1ccc2c(c1)c(-c1cnn(CCOCCOCCO)c1)nn2C1CCCCO1]

To a solution of [3-[1-[2-[2-(2-benzyloxyethoxy)ethoxy]ethyl]pyrazol-4-yl]-1-tetrahydro pyran-2-yl-indazol-5-yl]oxy-tert-butyl-dimethyl-silane (154 mg, 0.25 mmol) in ethanol (3.6 mL) at RT was added palladium hydroxide on carbon (20 mg). The reaction mixture was stirred under hydrogen atmosphere at RT for 6 h. The reaction mixture filtered and the filtrate was concentrated under reduced pressure. The residue was purified by silica gel column chromatography using dichloromethane/methanol 100/0 to 90/10 as eluent to afford 2-[2-[2-[4-[5-[tert-butyl(dimethyl)silyl]oxy-1-tetrahydropyran-2-yl-indazol-3-yl]pyrazol-1-yl]ethoxy] ethoxy]ethanol as a colorless oil.

LCMS method F: [M+H]⁺=531.4, t_{R}=3.10 min

### Preparation of Intermediate 358: 2-[2-[2-[4-[5-[tert-butyl(dimethyl)silyl]oxy-1-tetra hydropyran-2-yl-indazol-3-yl]pyrazol-1-yl]ethoxy]ethoxy]ethyl methanesulfonate

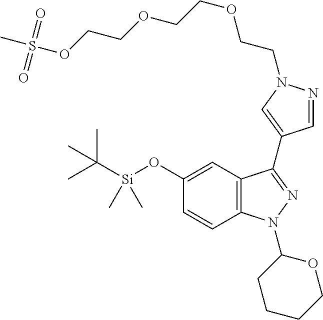 [SMILES: CC(C)(C)[Si](C)(C)Oc1ccc2c(c1)c(-c1cnn(CCOCCOCCOS(C)(=O)=O)c1)nn2C1CCCCO1]

To a solution of 2-[2-[2-[4-[5-[tert-butyl(dimethyl)silyl]oxy-1-tetrahydropyran-2-yl-indazol-3-yl]pyrazol-1-yl]ethoxy]ethoxy]ethanol (90 mg, 0.17 mmol) and triethylamine (47 μL, 0.34 mmol) in dichloromethane (2 mL) at 0° C. was added dropwise methanesulfonyl chloride (17 μL, 0.22 mmol). The reaction mixture was stirred at RT for 2 h. The reaction mixture was diluted with water and extracted with dichloromethane. The combined organic layers were washed with brine, dried over anhydrous magnesium sulfate, filtered and evaporated under reduced pressure to afford 2-[2-[2-[4-[5-[tert-butyl(dimethyl)silyl]oxy-1-tetrahydropyran-2-yl-indazol-3-yl]pyrazol-1-yl]ethoxy]ethoxy]ethyl methanesulfonate as a colorless oil which was used in the next step without further purification.

LCMS method F: [M+H]⁺=609.3, t_{R}=3.27 min

### Preparation of Intermediate 359: 19-(oxan-2-yl)-8,11,14-trioxa-4,5,19,20-tetraaza tetracyclo[13.5.2.1^{2,5}.0^{18,21}]tricosa-1(20),2(23),3,15(22),16,18(21)-hexaene

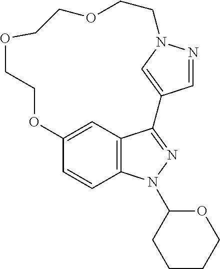 [SMILES: c1cc2c3cc1OCCOCCOCCn1cc(cn1)-c3nn2C1CCCCO1]

To a suspension of cesium carbonate (165 mg, 0.51 mmol) in anhydrous DMF (10 mL) at 70° C. was added dropwise 2-[2-[2-[4-[5-[tert-butyl(dimethyl)silyl]oxy-1-tetrahydropyran-2-yl-indazol-3-yl]pyrazol-1-yl]ethoxy]ethoxy]ethyl methanesulfonate (103 mg, 0.17 mmol) in DMF (4 mL). The reaction mixture was stirred at 70° C. for 6 h. The reaction mixture was filtered over a pad of Celite and washed with ethyl acetate. The filtrate was concentrated under reduced pressure. The residue was purified by silica gel column chromatography using cyclohexane/ethyl acetate 100/0 to 30/70 as an eluent to afford 19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene as a white foam.

LCMS method F: [M+H]⁺=399.3, t_{R}=2.41 min

### Preparation of Example 63: 8,11,14-trioxa-4,5,19,20-tetraazatetracyclo[13.5.2.1^{2,5}.0^{18,21}]tricosa-1(20),2(23),3,15(22),16,18(21)-hexaene

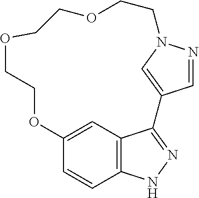 [SMILES: c1cc2[nH]nc3c2cc1OCCOCCOCCn1cc-3cn1]

To a solution of 19-(oxan-2-yl)-8,11,14-trioxa-4,5,19,20-tetraazatetracyclo[13.5.2.1^{2,5}.0^{18,21}] tricosa-1(20),2(23),3,15(22),16,18(21)-hexaene (62 mg, 0.16 mmol) in dichloromethane (1 mL) at RT was added TFA (240 μL, 3.20 mmol). The reaction mixture was stirred at RT for 6 h. The reaction mixture was concentrated under reduced pressure, diluted with a saturated aqueous NaHCO₃ solution and extracted with ethyl acetate. The combined organic layers were dried over anhydrous sodium sulfate, filtered and evaporated under reduced pressure. The residue was purified by silica gel column chromatography using dichloromethane/methanol 100/0 to 97/3 as eluent to afford 8,11,14-trioxa-4,5,19,20-tetraazatetracyclo[13.5.2.1^{2,5}.0^{18,21}] tricosa-1(20),2(23),3,15(22),16,18(21)-hexaene as a solid.

LCMS method F: [M+H]⁺=315.3, t_{R}=1.80 min

LCMS method G: [M+H]⁺=315.3, t_{R}=1.82 min

1H NMR (400 MHz, d6-DMSO) 12.73 (1H, s), 8.48 (1H, d, J=0.6 Hz), 7.92 (1H, d, J=2.3 Hz), 7.81 (1H, d, J=0.6 Hz), 7.38 (1H, d, J=8.4 Hz), 7.00 (1H, dd, J=2.3, 8.9 Hz), 4.41-4.32 (4H, m), 3.82-3.76 (4H, m), 3.68 (4H, s) ppm.

### Example 64: (6S)-6-methyl-8,11,14-trioxa-4,5,19,20-tetraazatetracyclo[13.5.2.1^{2,5}.0^{18,21}] tricosa-1(20),2(23),3,15(22),16,18(21)-hexaene

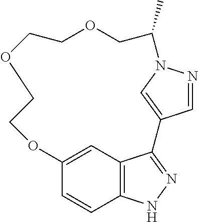 [SMILES: C[C@H]1COCCOCCOc2ccc3[nH]nc(c3c2)-c2cnn1c2]

Example 64 is prepared according to the synthesis route described in general Scheme D.

### Preparation of Intermediate 360: [(1R)-2-benzyloxy-1-methyl-ethyl]methane sulfonate

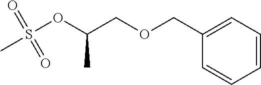 [SMILES: C[C@H](COCc1ccccc1)OS(C)(=O)=O]

To a solution of (2R)-1-benzyloxypropan-2-ol (350 mg, 2.10 mmol) in dichloromethane (12.5 mL) at 0° C. was added triethylamine (0.38 mL, 2.74 mmol) and methanesulfonyl chloride (0.18 mL, 2.31 mmol). The reaction mixture was stirred at 0° C. for 10 min and at RT for 5 h. Water was added and the organic layer was washed with a saturated aqueous NaHCO₃ solution and saturated aqueous ammonium chloride solution. The organic layer was dried over anhydrous sodium sulfate, filtered and evaporated under reduced pressure to afford [(1R)-2-benzyloxy-1-methyl-ethyl]methanesulfonate as a yellow oil which was used in the next step without further purification.

LCMS method F: [M+H]⁺=245.2, t_{R}=2.27 min

### Preparation of Intermediate 361: 1-[(1S)-2-benzyloxy-1-methyl-ethyl]-4-bromo-pyrazole

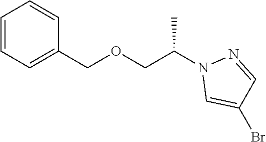 [SMILES: C[C@@H](COCc1ccccc1)n1cc(Br)cn1]

To a solution of 4-bromo-1H-pyrazole (230 mg, 1.57 mmol) in acetonitrile (7 mL) was added cesium carbonate (665 mg, 2.04 mmol) and [(1R)-2-benzyloxy-1-methyl-ethyl]methane sulfonate (421 mg, 1.72 mmol). The reaction mixture was stirred at 85° C. for 2 h. The reaction mixture was cooled to RT and water and ethyl acetate were added. The aqueous layer was extracted with ethyl acetate and the combined organic layers were dried over anhydrous sodium sulfate, filtered and evaporated under reduced pressure to afford 1-[(1S)-2-benzyloxy-1-methyl-ethyl]-4-bromo-pyrazole as a yellow oil which was used in the next step without further purification.

LCMS method F: [M+H]⁺=295.1-297.1, t_{R}=2.77 min

### Preparation of Intermediate 362: (2S)-2-(4-bromopyrazol-1-yl)propan-1-ol

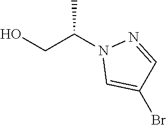

To a solution of 1-[(1S)-2-benzyloxy-1-methyl-ethyl]-4-bromo-pyrazole (462 mg, 1.57 mmol) in ethanol (10 mL) was added concentrated aqueous HCl solution (37% w.) (7.70 mL). The resulting mixture was stirred at 80° C. for 72 h. The reaction mixture was cooled to RT and the solvent was removed under reduced pressure. The residue was diluted with a saturated aqueous NaHCO₃ solution and ethyl acetate. The aqueous layer was extracted with ethyl acetate and the combined organic layers were dried over anhydrous sodium sulfate, filtered and evaporated under reduced pressure. The residue was purified by silica gel column chromatography using cyclohexane/ethyl acetate 100/0 to 60/40 as eluent to afford (2S)-2-(4-bromopyrazol-1-yl)propan-1-ol as a colorless oil.

LCMS method F: [M+H]⁺=205.0-207.0, t_{R}=1.57 min

### Preparation of Intermediate 363: 1-[(1S)-2-[2-(2-benzyloxyethoxy)ethoxy]-1-methyl-ethyl]-4-bromo-pyrazole

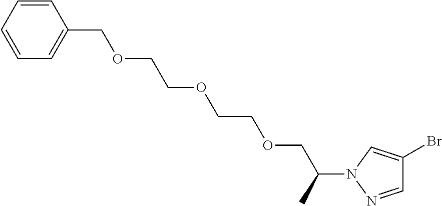 [SMILES: C[C@@H](COCCOCCOCc1ccccc1)n1cc(Br)cn1]

To a solution of (2S)-2-(4-bromopyrazol-1-yl)propan-1-ol (200 mg, 0.98 mmol) in dry DMF (2.5 mL) was added sodium hydride (60% dispersion in mineral oil) (58 mg, 1.46 mmol). The reaction mixture was stirred at RT for 10 min and a solution of 2-(2-benzyloxyethoxy)ethyl methanesulfonate (intermediate 145) (400 mg, 1.46 mmol) in dry DMF (1.5 mL) was added dropwise. The reaction mixture was stirred at RT for 5 h. The reaction mixture was diluted with water and ethyl acetate was added. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 60/40 as eluent to afford 1-[(1S)-2-[2-(2-benzyloxyethoxy)ethoxy]-1-methyl-ethyl]-4-bromo-pyrazole as a pale yellow oil.

LCMS method F: [M+H]⁺=383.2-385.2, t_{R}=2.74 min

### Preparation of Intermediate 364: [3-[1-[(1S)-2-[2-(2-benzyloxyethoxy)ethoxy]-1-methyl-ethyl]pyrazol-4-yl]-1-tetrahydropyran-2-yl-indazol-5-yl]oxy-tert-butyl-dimethyl-silane

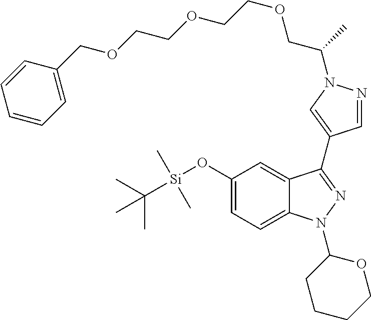 [SMILES: C[C@@H](COCCOCCOCc1ccccc1)n1cc(-c2nn(C3CCCCO3)c3ccc(O[Si](C)(C)C(C)(C)C)cc23)cn1]

To a suspension of 1-[(1S)-2-[2-(2-benzyloxyethoxy)ethoxy]-1-methyl-ethyl]-4-bromo-pyrazole (295 mg, 0.77 mmol) in dioxane (5 mL) and water (0.25 mL) was added tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (459 mg, 1 mmol) and potassium phosphate tribasic (490 mg, 2.31 mmol). The reaction mixture was purged by with argon for 10 min then tetrakis(triphenylphosphine)palladium(0) (44 mg, 0.04 mmol) and Xphos (36 mg, 0.08 mmol) were added. The reaction mixture was stirred at 90° C. for 4 h. The reaction mixture was cooled to RT and filtered over a pad a Celite. The filtrate was diluted with water and ethyl acetate. The phases were separated and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 40/60 as eluent to afford [3-[1-[(1S)-2-[2-(2-benzyloxyethoxy)ethoxy]-1-methyl-ethyl]pyrazol-4-yl]-1-tetrahydropyran-2-yl-indazol-5-yl]oxy-tert-butyl-dimethyl-silane as a pink oil.

LCMS method F: [M+H]⁺=635.5, t_{R}=3.70 min

### Preparation of Intermediate 365: 2-[2-[(2S)-2-[4-[5-[tert-butyl(dimethyl)silyl]oxy-1-tetrahydro pyran-2-yl-indazol-3-yl]pyrazol-1-yl]propoxy]ethoxy]ethanol

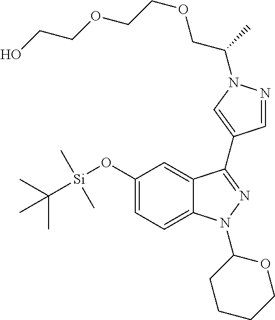 [SMILES: C[C@@H](COCCOCCO)n1cc(-c2nn(C3CCCCO3)c3ccc(O[Si](C)(C)C(C)(C)C)cc23)cn1]

To a suspension of [3-[1-[(1S)-2-[2-(2-benzyloxyethoxy)ethoxy]-1-methyl-ethyl]pyrazol-4-yl]-1-tetrahydropyran-2-yl-indazol-5-yl]oxy-tert-butyl-dimethyl-silane (260 mg, 0.41 mmol) in ethanol (3.4 mL) under argon was added palladium on charcoal 10% (26 mg). The reaction mixture was stirred under hydrogen atmosphere at RT overnight. The mixture was filtered over a pad of Celite and washed with ethanol and ethyl acetate. The filtrate were evaporated under reduced pressure to afford 2-[2-[(2S)-2-[4-[5-[tert-butyl(dimethyl)silyl]oxy-1-tetrahydro pyran-2-yl-indazol-3-yl]pyrazol-1-yl]propoxy]ethoxy]ethanol as a yellow oil which was used in the next step without further purification.

LCMS method F: [M+H]⁺=545.5, t_{R}=3.21 min

### Preparation of Intermediate 366: 2-[2-[(2S)-2-[4-[5-[tert-butyl(dimethyl)silyl]oxy-1-tetrahydropyran-2-yl-indazol-3-yl]pyrazol-1-yl]propoxy]ethoxy]ethylmethane sulfonate

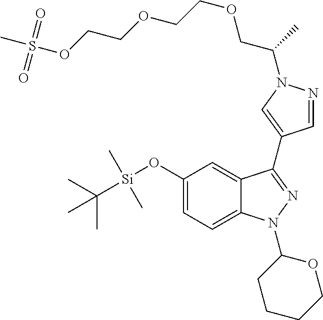 [SMILES: C[C@@H](COCCOCCOS(C)(=O)=O)n1cc(-c2nn(C3CCCCO3)c3ccc(O[Si](C)(C)C(C)(C)C)cc23)cn1]

To a solution of 2-[2-[(2S)-2-[4-[5-[tert-butyl(dimethyl)silyl]oxy-1-tetrahydropyran-2-yl-indazol-3-yl]pyrazol-1-yl]propoxy]ethoxy]ethanol (190 mg, 0.35 mmol) in dichloromethane (2 mL) at 0° C. was added triethylamine (98 μL, 0.70 mmol) and methanesulfonyl chloride (35 μL, 0.46 mmol). The reaction mixture was stirred at 0° C. for 10 min then at RT for 4 h. Water was added and the organic layer was washed with a saturated aqueous NaHCO₃ solution then saturated aqueous NH₄Cl solution. The organic layer was dried over anhydrous sodium sulfate, filtered and the evaporated under reduced pressure to afford 2-[2-[(2S)-2-[4-[5-[tert-butyl(dimethyl)silyl]oxy-1-tetrahydropyran-2-yl-indazol-3-yl]pyrazol-1-yl]propoxy]ethoxy] ethylmethanesulfonate as an orange oil which was used in the next step without further purification.

LCMS method F: [M+H]⁺=623.5, t_{R}=3.38 min

### Preparation of Intermediate 367: (6S)-6-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene

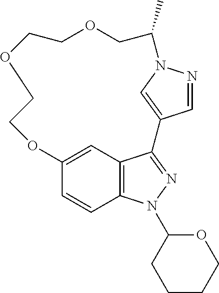 [SMILES: C[C@H]1COCCOCCOc2ccc3c(c2)c(nn3C2CCCCO2)-c2cnn1c2]

To a solution of cesium carbonate (340 mg, 1.04 mmol) in dry DMF (20 mL) at 60° C. was added dropwise a solution of 2-[2-[(2S)-2-[4-[5-[tert-butyl(dimethyl)silyl]oxy-1-tetrahydropyran-2-yl-indazol-3-yl]pyrazol-1-yl]propoxy]ethoxy]ethyl methanesulfonate (217 mg, 0.35 mmol) in dry DMF (8 mL). The reaction mixture was stirred at 60° C. for 6 h. The mixture was cooled to RT, filtered over a pad of Celite and washed with ethyl acetate. The solvent was evaporated under reduced pressure. The residue was purified by silica gel column chromatography using cyclohexane/ethyl acetate 100/0 to 0/100 as eluent. to afford (6S)-6-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16, 18(21)-hexaene as a colorless oil.

LCMS method F: [M+H]⁺=413.4, t_{R}=2.51 min

### Preparation of Example 64: (6S)-6-methyl-8,11,14-trioxa-4,5,19,20-tetraazatetra cyclo[13.5.2.1^{2,5}.0^{18,21}]tricosa-1(20),2(23),3,15(22),16,18(21)-hexaene

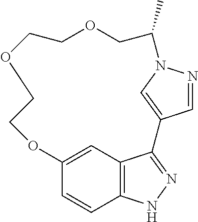 [SMILES: C[C@H]1COCCOCCOc2ccc3[nH]nc(c3c2)-c2cnn1c2]

To a solution of (6S)-6-methyl-19-(oxan-2-yl)-8,11,14-trioxa-4,5,19,20-tetraazatetracyclo [13.5.2.1^{2,5}.0^{18,21}]tricosa-1(20),2(23),3,15(22),16,18(21)-hexaene (72 mg, 0.17 mmol) in methanol (3.3 mL) and water (0.5 mL) was added p-toluenesulfonic acid monohydrate (165 mg, 0.87 mmol). The reaction mixture was stirred at 65° C. for 4 h. The reaction mixture was cooled to RT and quenched with a saturated aqueous NaHCO₃ solution until pH basic. ethyl acetate was added and the phases were separated. The aqueous layer was extracted with ethyl acetate and the combined organic layers were washed with brine, dried over anhydrous sodium sulfate, filtered and evaporated under reduced pressure. The residue was purified by silica gel column chromatography using dichloromethane/methanol 100/0 to 96/4 as eluent to afford (6S)-6-methyl-8,11,14-trioxa-4,5,19,20-tetraazatetracyclo[13.5.2.1^{2,5}.0^{18,21}]tricosa-1(20), 2(23),3,15(22),16,18(21)-hexaene as a solid.

LCMS method F: [M+H]⁺=329.3, t_{R}=1.93 min

LCMS method G: [M+H]⁺=329.3, t_{R}=1.93 min 1H NMR (400 MHz, d6-DMSO) 12.72 (1H, s), 8.47 (1H, s), 7.91-7.89 (1H, m), 7.81 (1H, s), 7.38 (1H, d, J=8.9 Hz), 6.99 (1H, dd, J=2.3, 8.9 Hz), 4.66-4.59 (1H, m), 4.40-4.26 (2H, m), 3.81-3.61 (8H, m), 1.52 (3H, d, J=7.0 Hz) ppm.

### Example 65: (6R)-6-methyl-8,11,14-trioxa-4,5,19,20-tetraazatetracyclo[13.5.2.1^{2,5}.0^{18,21}] tricosa-1(20),2(23),3,15(22),16,18(21)-hexaene

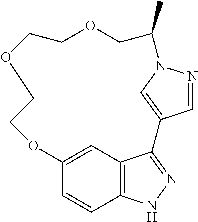 [SMILES: C[C@@H]1COCCOCCOc2ccc3[nH]nc(c3c2)-c2cnn1c2]

Example 65 is prepared according to the synthesis route described in general Scheme D, following the same synthesis procedures as for Example 64.

### Preparation of Intermediate 368: (6R)-6-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene

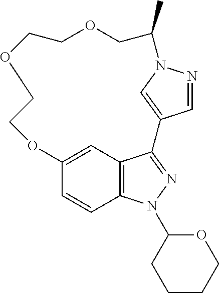 [SMILES: C[C@@H]1COCCOCCOc2ccc3c(c2)c(nn3C2CCCCO2)-c2cnn1c2]

To a solution of cesium carbonate (537 mg, 1.65 mmol) in dry DMF (30 mL) at 60° C. was added dropwise a solution of 2-[2-[(2R)-2-[4-[5-[tert-butyl(dimethyl)silyl]oxy-1-tetrahydropyran-2-yl-indazol-3-yl]pyrazol-1-yl]propoxy]ethoxy]ethyl methanesulfonate (343 mg, 0.55 mmol) in dry DMF (12 mL). The reaction mixture was stirred at 60° C. for 6 h. The mixture was cooled to RT, filtered over a pad of Celite and washed with ethyl acetate. The solvent was evaporated under reduced pressure. The residue was purified by silica gel column chromatography using cyclohexane/ethyl acetate 100/0 to 0/100 as eluent. to afford (6R)-6-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16, 18(21)-hexaene as a colorless oil.

LCMS method F: [M+H]⁺=413.4, t_{R}=2.51 min

### Preparation of Example 65: (6R)-6-methyl-8,11,14-trioxa-4,5,19,20-tetraazatetra cyclo[13.5.2.1^{2,5}.0^{18,21}]tricosa-1(20),2(23),3,15(22),16,18(21)-hexaene

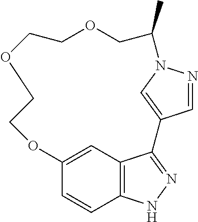 [SMILES: C[C@@H]1COCCOCCOc2ccc3[nH]nc(c3c2)-c2cnn1c2]

To a solution of (6R)-6-methyl-19-(oxan-2-yl)-8,11,14-trioxa-4,5,19,20-tetraazatetracyclo [13.5.2.1^{2,5}.0^{18,21}] tricosa-1(20),2(23),3,15(22),16,18(21)-hexaene (100 mg, 0.24 mmol) in methanol (4.5 mL) and water (0.6 mL) was added p-toluenesulfinic acid monohydrate (228 mg, 1.20 mmol). The reaction mixture was stirred at 65° C. for 4 h. The reaction mixture was cooled to RT and quenched with a saturated aqueous NaHCO₃ solution until pH basic. ethyl acetate was added and the phases were separated. The aqueous layer was extracted with ethyl acetate and the combined organic layers were washed with brine, dried over anhydrous sodium sulfate, filtered and evaporated under reduced pressure. The residue was purified by silica gel column chromatography using dichloromethane/methanol 100/0 to 96/4 as eluent to afford (6R)-6-methyl-8,11,14-trioxa-4,5,19,20-tetraazatetracyclo[13.5.2.1^{2,5}.0^{18,21}]tricosa-1(20),2(23),3, 15(22),16,18(21)-hexaene as a solid.

LCMS method F: [M+H]⁺=329.3, t_{R}=1.93 min

LCMS method G: [M+H]⁺=329.3, t_{R}=1.93 min

1H NMR (400 MHz, d6-DMSO) 12.72 (1H, s), 8.48-8.47 (1H, m), 7.90 (1H, d, J=2.1 Hz), 7.81-7.80 (1H, m), 7.38 (1H, d, J=8.9 Hz), 6.99 (1H, dd, J=2.3, 8.9 Hz), 4.65-4.59 (1H, m), 4.40-4.26 (2H, m), 3.81-3.62 (8H, m), 1.53-1.50 (3H, m) ppm.

### Example 66: (13S)-13-methyl-7,10,14-trioxa-23-thia-4,19,20-triazatetracyclo[13.5.2.1^{2,5}.0^{18,21}]tricosa-1(20),2,4,15(22),16,18(21)-hexaene

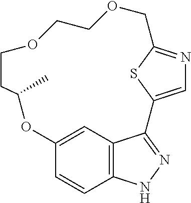 [SMILES: C[C@H]1CCOCCOCc2ncc(s2)-c2n[nH]c3ccc(cc23)O1]

Example 66 is prepared according to the synthesis route described in general Scheme D.

### Preparation of Intermediate 369: 2-[2-[(3R)-3-benzyloxybutoxy] ethoxymethyl]-5-bromo-thiazole

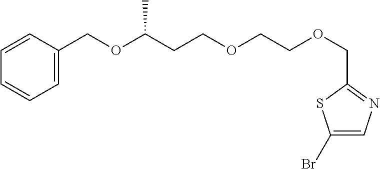 [SMILES: C[C@H](CCOCCOCc1ncc(Br)s1)OCc1ccccc1]

To a suspension of (5-bromothiazol-2-yl)methanol (722 mg, 3.72 mmol) and cesium carbonate (6.06 g, 18.6 mmol) in dry acetonitrile (10 mL) was added a solution of 2-[(3R)-3-benzyloxybutoxy]ethyl 4-methylbenzenesulfonate (intermediate 346) (1.68 g, 4.46 mmol) in dry acetonitrile (14 mL). The reaction mixture was stirred at 70° C. for 19 h. The reaction mixture was filtered and the filtrate was diluted with brine and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 80/20 as eluent to afford 2-[2-[(3R)-3-benzyloxybutoxy] ethoxymethyl]-5-bromo-thiazole as a yellow oil.

LCMS method F: [M+H]⁺=400.1-402.2, t_{R}=3.02 min

### Preparation of Intermediate 370: [3-[2-[2-[(3R)-3-benzyloxybutoxy]ethoxymethyl] thiazol-5-yl]-1-tetrahydropyran-2-yl-indazol-5-yl]oxy-tert-butyl-dimethyl-silane

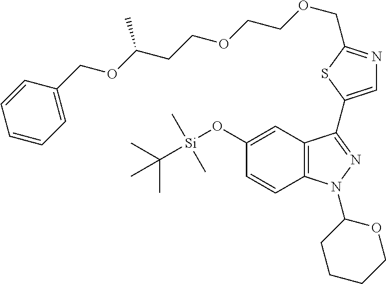 [SMILES: C[C@H](CCOCCOCc1ncc(-c2nn(C3CCCCO3)c3ccc(O[Si](C)(C)C(C)(C)C)cc23)s1)OCc1ccccc1]

To a degassed suspension of 2-[2-[(3R)-3-benzyloxybutoxy]ethoxymethyl]-5-bromo-thiazole (720 mg, 1.8 mmol), tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (990 mg, 2.16 mmol), potassium phosphate tribasic (1.146 g, 5.4 mmol), and XPhos (86 mg, 0.18 mmol) in dioxane (13 mL) and water (1.3 mL), was added tetrakis(triphenylphosphine)palladium(0) (104 mg, 0.09 mmol). The reaction mixture was stirred under microwave irradiations at 90° C. for 2 h. The reaction was cooled to RT then filtered. The filtrate was diluted with brine and extracted with ethyl acetate. The combined organic layers were dried over anhydrous sodium sulfate, filtered and evaporated under reduced pressure. The residue was purified by silica gel column chromatography using with cyclohexane/ethyl acetate 100/0 to 80/20 as eluent to afford [3-[2-[2-[(3R)-3-benzyloxybutoxy]ethoxymethyl]thiazol-5-yl]-1-tetrahydropyran-2-yl-indazol-5-yl]oxy-tert-butyl-dimethyl-silane as a pale yellow oil.

LCMS method F: [M+H]⁺=652.5, t_{R}=3.95 min

### Preparation of Intermediate 371: (2R)-4-[2-[[5-[5-[tert-butyl(dimethyl)silyl]oxy-1-tetrahydropyran-2-yl-indazol-3-yl]thiazol-2-yl]methoxy]ethoxy]butan-2-ol

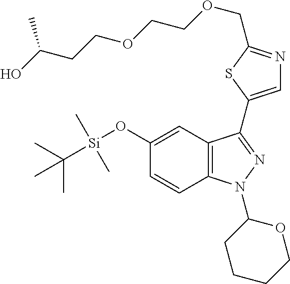 [SMILES: C[C@@H](O)CCOCCOCc1ncc(-c2nn(C3CCCCO3)c3ccc(O[Si](C)(C)C(C)(C)C)cc23)s1]

To a solution of [3-[2-[2-[(3R)-3-benzyloxybutoxy]ethoxymethyl]thiazol-5-yl]-1-tetrahydro pyran-2-yl-indazol-5-yl]oxy-tert-butyl-dimethyl-silane (290 mg, 0.445 mmol) in dichloromethane (8.9 mL) and a pH 7 phosphate buffer (0.45 mL) at RT was added 2,3-dichloro-5,6-dicyano-p-benzoquinone (253 mg, 1.11 mmol). The reaction mixture was stirred at RT for 18 h. The reaction mixture was diluted with a saturated NaHCO₃ solution and extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 40/60 as eluent to afford (2R)-4-[2-[[5-[5-[tert-butyl(dimethyl)silyl]oxy-1-tetrahydropyran-2-yl-indazol-3-yl]thiazol-2-yl]methoxy]ethoxy]butan-2-ol as a pale yellow oil.

LCMS method F: [M+H]⁺=562.4, t_{R}=3.48 min

### Preparation of Intermediate 372: [(1R)-3-[2-[[5-[5-[tert-butyl(dimethyl)silyl]oxy-1-tetrahydropyran-2-yl-indazol-3-yl]thiazol-2-yl]methoxy]ethoxy]-1-methyl-propyl] methanesulfonate

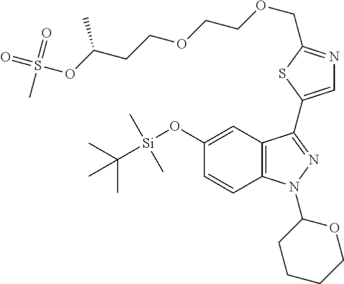 [SMILES: C[C@H](CCOCCOCc1ncc(-c2nn(C3CCCCO3)c3ccc(O[Si](C)(C)C(C)(C)C)cc23)s1)OS(C)(=O)=O]

To a cooled solution at 0° C. of (2R)-4-[2-[[5-[5-[tert-butyl(dimethyl)silyl]oxy-1-tetrahydro pyran-2-yl-indazol-3-yl]thiazol-2-yl]methoxy]ethoxy]butan-2-ol (110 mg, 0.196 mmol) and triethylamine (68 μL, 0.490 mmol) in dichloromethane (3 mL) was added methanesulfonyl chloride (30 μL, 0.392 mmol). The reaction mixture was stirred at RT for 16 h. Additional methanesulfonyl chloride (30 μL, 0.392 mmol), triethylamine (68 μL, 0.490 mmol) and dichloromethane (1 mL) were added and the reaction mixture was stirred at RT for 16 h. Additional methanesulfonyl chloride (12 μL, 0.152 mmol), triethylamine (85 μL, 0.606 mmol) and dichloromethane (4 mL) were added and the reaction mixture was stirred at RT for 16 h. Additional methanesulfonyl chloride (12 μL, 0.152 mmol) and triethylamine (85 μL, 0.606 mmol) were added and the reaction mixture was stirred at RT for 3 h. The reaction mixture was diluted with brine and extracted with dichloromethane. The combined organic layers were dried over anhydrous sodium sulfate, filtered and evaporated under reduced pressure to afford [(1R)-3-[2-[[5-[5-[tert-butyl(dimethyl)silyl]oxy-1-tetrahydropyran-2-yl-indazol-3-yl]thiazol-2-yl]methoxy]ethoxy]-1-methyl-propyl] methanesulfonate as a pale yellow oil which was used in the next step without further purification.

LCMS method F: [M+H]⁺=640.4, t_{R}=3.58 min

### Preparation of Intermediate 373: (13S)-13-methyl-19-(oxan-2-yl)-7,10,14-trioxa-23-thia-4,19,20-triazatetracyclo[13.5.2.1^{2,5}.0^{18,21}]tricosa-1(20),2,4,15(22),16,18(21)-hexaene

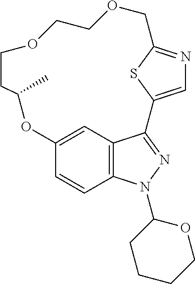 [SMILES: C[C@H]1CCOCCOCc2ncc(s2)-c2nn(C3CCCCO3)c3ccc(cc23)O1]

To a suspension of cesium carbonate (318 mg, 0.976 mmol) in anhydrous DMF (100 mL) at 80° C. was added dropwise a solution of [(1R)-3-[2-[[5-[5-[tert-butyl(dimethyl)silyl]oxy-1-tetrahydropyran-2-yl-indazol-3-yl]thiazol-2-yl]methoxy]ethoxy]-1-methyl-propyl]methane sulfonate (156 mg, 0.244 mmol) in anhydrous DMF (100 mL). The reaction mixture was stirred at 80° C. for 4 h. The solvent was evaporated under reduced pressure and the residue was diluted with brine and extracted with ethyl acetate. The combined organic layers were dried over anhydrous sodium sulfate, filtered and evaporated under reduced pressure. The residue was purified by silica gel column chromatography using cyclohexane/(ethyl acetate/ethanol (3/1)) 100/0 to 80/20 as eluent to afford (13S)-13-methyl-19-(oxan-2-yl)-7,10,14-trioxa-23-thia-4,19,20-triazatetracyclo[13.5.2.1^{2,5}.0^{18,21}]tricosa-1(20),2,4,15(22),16,18(21)-hexaene as a colorless oil.

LCMS method F: [M+H]⁺=430.4, t_{R}=2.90 min

### Preparation of Example 66: (13S)-13-methyl-7,10,14-trioxa-23-thia-4,19,20-triaza tetracyclo[13.5.2.1^{2,5}.0^{18,21}]tricosa-1(20),2,4,15(22),16,18(21)-hexaene

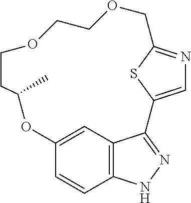 [SMILES: C[C@H]1CCOCCOCc2ncc(s2)-c2n[nH]c3ccc(cc23)O1]

To a solution of (13S)-13-methyl-19-(oxan-2-yl)-7,10,14-trioxa-23-thia-4,19,20-triazatetra cyclo[13.5.2.1^{2,5}.0^{18,21}]tricosa-1(20),2,4,15(22),16,18(21)-hexaene (19 mg, 0.044 mmol) in methanol (2.6 mL) and water (0.35 mL) at RT was added p-toluenesulfonic acid monohydrate (42 mg, 0.22 mmol). The reaction mixture was stirred at 65° C. for 16 h. The reaction mixture was concentrated under reduced pressure and the residue was neutralized by slow addition of saturated aqueous NaHCO₃ solution. The reaction mixture was diluted with ethyl acetate and the phases were separated. The aqueous phase was extracted with ethyl acetate. The combined organic layers were washed with brine, dried over anhydrous sodium sulfate, filtered and evaporated reduced pressure. The residue was purified by preparative TLC eluting with dichloromethane/methanol 95/5 to afford (13S)-13-methyl-7,10,14-trioxa-23-thia-4,19,20-triazatetracyclo[13.5.2.1^{2,5}.0^{18,21}]tricosa-1(20),2,4,15(22),16,18(21)-hexaene as a solid.

LCMS method F: [M+H]⁺=346, t_{R}=2.25 min

LCMS method G: [M+H]⁺=346, t_{R}=2.23 min

1H NMR (400 MHz, MeOD) 8.00 (1H, s), 7.65 (1H, d, J=2.5 Hz), 7.48-7.45 (1H, m), 7.07-7.04 (1H, dd, J=2.3, 9.1 Hz), 5.08-5.04 (1H, d, J=15.6 Hz), 4.73-4.69 (1H, d, J=15.5 Hz), 4.63-4.55 (1H, m), 3.89-3.79 (2H, m), 3.76-3.70 (3H, m), 3.59-3.53 (1H, m), 2.68-2.61 (1H, m), 1.61-1.54 (1H, m), 1.45 (3H, d, J=5.7 Hz) ppm.

### Example 67: (12R)-12-methyl-8,11,14-trioxa-4,5,19,20-tetraazatetracyclo[13.5.2.1^{2,5}.0^{18,21}]tricosa-1(20),2(23),3,15(22),16,18(21)-hexaene

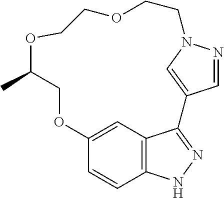 [SMILES: C[C@@H]1COc2ccc3[nH]nc(c3c2)-c2cnn(c2)CCOCCO1]

Example 67 is prepared according to the synthesis route described in general Scheme D, following the same synthesis procedures as for Example 54.

### Preparation of Intermediate 374: (12R)-12-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene

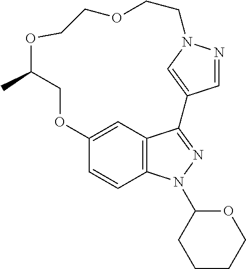 [SMILES: C[C@@H]1COc2ccc3c(c2)c(nn3C2CCCCO2)-c2cnn(c2)CCOCCO1]

To a suspension of cesium carbonate (502 mg, 1.54 mmol) in dry DMF (40 mL) at 60° C. was added dropwise a solution of [(2R)-2-[2-[2-[4-[5-[tert-butyl(dimethyl)silyl]oxy-1-tetrahydro pyran-2-yl-indazol-3-yl]pyrazol-1-yl]ethoxy]ethoxy]propyl] methanesulfonate (400 mg, 0.514 mmol) in dry DMF (16 mL). The reaction mixture was stirred at 60° C. for 72 h. The solvent was evaporated under reduced pressure and the residue was purified by silica gel column chromatography using cyclohexane/(ethyl acetate/ethanol (3-1)) 90/10 to 50/50 as eluent to afford (12R)-12-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene as a colorless oil.

LCMS method F: [M+H]⁺=413.3, t_{R}=2.49 min

### Preparation of Example 67: (12R)-12-methyl-8,11,14-trioxa-4,5,19,20-tetraaza tetracyclo[13.5.2.1^{2,5}.0^{18,21}]tricosa-1(20),2(23),3,15(22),16,18(21)-hexaene

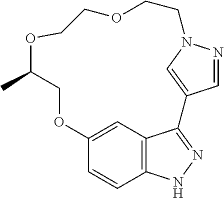 [SMILES: C[C@@H]1COc2ccc3[nH]nc(c3c2)-c2cnn(c2)CCOCCO1]

To a solution of (12R)-12-methyl-19-(oxan-2-yl)-8,11,14-trioxa-4,5,19,20-tetraaza tetracyclo [13.5.2.1^{2,5}.0^{18,21}]tricosa-1(20),2(23),3,15(22),16,18(21)-hexaene (40 mg, 0.097 mmol) in methanol (3.5 mL) and water (0.5 mL) was added p-toluenesulfonic acid monohydrate (55 mg, 0.36 mmol). The reaction mixture was stirred at RT overnight. The solvent was removed under reduced pressure. The residue was diluted with a saturated aqueous potassium carbonate solution and extracted with dichloromethane. The organic layer was washed with brine, dried over anhydrous sodium sulfate, filtered and evaporated under reduced pressure. The resulting solid was triturated with diethyl ether, filtered and dried to afford (12R)-12-methyl-8,11,14-trioxa-4,5,19,20-tetraazatetracyclo[13.5.2.1^{2,5}.0^{18,21}]tricosa-1(20),2(23),3,15(22),16,18(21)-hexaene as a powder.

LCMS method F: [M+H]⁺=329.3, t_{R}=1.93 min

LCMS method G: [M+H]⁺=329.3, t_{R}=1.93 min

1H NMR (400 MHz, CDCl₃) 8.55-8.54 (1H, m), 8.02 (1H, s), 7.95 (1H, d, J=2.1 Hz), 7.35 (1H, d, J=19.4 Hz), 7.11 (1H, dd, J=2.3, 8.9 Hz), 4.50-4.46 (2H, m), 4.36-4.33 (2H, m), 3.91-3.82 (4H, m), 3.78-3.58 (4H, m), 1.37-1.34 (3H, m) ppm.

### Example 68: 9,12,15-trioxa-4,20,21-triazatetracyclo[14.5.2.1^{2,6}.0^{19,22}]tetracosa-1(21), 2(24),3,5,16(23),17,19(22)-heptaene

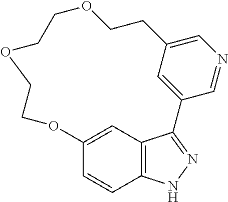 [SMILES: c1ncc2cc1CCOCCOCCOc1ccc3[nH]nc-2c3c1]

Example 68 is prepared according to the synthesis route described in general Scheme D.

### Preparation of Intermediate 375: 3-(2-{2-[2-(benzyloxy)ethoxy]ethoxy}ethyl)-5-bromopyridine

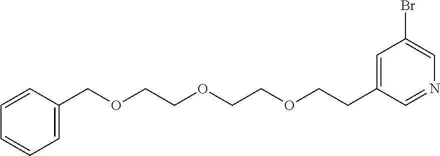 [SMILES: Brc1cncc(CCOCCOCCOCc2ccccc2)c1]

To a solution of 2-(5-bromopyridin-3-yl)ethan-1-ol (300 mg, 1.48 mmol) in DMF (5 mL) at 0° C. was added sodium hydride (60% dispersion in mineral oil) (89 mg, 2.23 mmol) and 2-(2-benzyloxyethoxy)ethyl methanesulfonate (intermediate 145) (1.222 g, 4.45 mmol) in DMF (3 mL). The reaction mixture was stirred at RT for 16 h. The reaction mixture was diluted with ethyl acetate and washed with water then brine. The organic layer was dried over anhydrous sodium sulfate, filtered and evaporated reduced pressure. The residue was purified by silica gel column chromatography using cyclohexane/ethyl acetate 100/0 to 70/30 as eluent to afford 3-(2-{2-[2-(benzyloxy)ethoxy]ethoxy}ethyl)-5-bromopyridine as a light yellow oil.

LCMS method F: [M+H]⁺=380.3-382.3, t_{R}=2.58 min

### Preparation of Intermediate 376: [3-[5-[2-[2-(2-benzyloxyethoxy)ethoxy]ethyl]-3-pyridyl]-1-tetrahydropyran-2-yl-indazol-5-yl]oxy-tert-butyl-dimethyl-silane

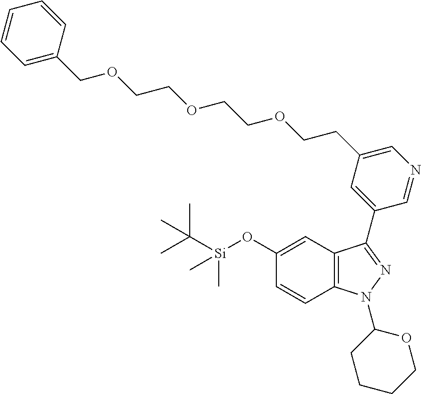 [SMILES: CC(C)(C)[Si](C)(C)Oc1ccc2c(c1)c(-c1cncc(CCOCCOCCOCc3ccccc3)c1)nn2C1CCCCO1]

To a suspension of 3-[2-[2-(2-benzyloxyethoxy)ethoxy]ethyl]-5-bromo-pyridine (477 mg, 1.25 mmol) in dioxane (2.2 mL) and water (0.3 mL) was added tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (748 mg, 1.63 mmol), and potassium phosphate tribasic (799 mg, 3.76 mmol). The reaction mixture was purged argon for 15 min then tetrakis(triphenylphosphine) palladium(0) (72 mg, 0.06 mmol) and Xphos (60 mg, 0.13 mmol) were added. The reaction mixture was heated under microwave irradiations at 90° C. for 1 h. The reaction mixture was poured into water and extracted with ethyl acetate. The combined organic layers were washed with brine, dried over anhydrous sodium sulfate and evaporated under reduced pressure. The residue was purified by silica gel column chromatography using cyclohexane/ethyl acetate 80/20 as eluent to afford [3-[5-[2-[2-[(2R)-2-benzyloxypropoxy]ethoxy]ethyl]-3-pyridyl]-1-tetrahydropyran-2-yl-indazol-5-yl]oxy-tert-butyl-dimethyl-silane was afford as a light yellow oil.

LCMS method F: [M+H]⁺=632.6, t_{R}=3.55 min

### Preparation of Intermediate 377: 2-[2-[2-[5-[5-[tert-butyl(dimethyl)silyl]oxy-1-tetrahydropyran-2-yl-indazol-3-yl]-3-pyridyl]ethoxy]ethoxy]ethanol

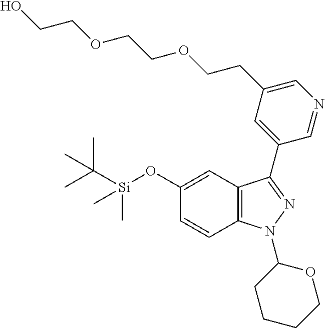 [SMILES: CC(C)(C)[Si](C)(C)Oc1ccc2c(c1)c(-c1cncc(CCOCCOCCO)c1)nn2C1CCCCO1]

To a solution of [3-[5-[2-[2-[(2R)-2-benzyloxypropoxy]ethoxy]ethyl]-3-pyridyl]-1-tetrahydro pyran-2-yl-indazol-5-yl]oxy-tert-butyl-dimethyl-silane (365 mg, 0.58 mmol) in ethanol (5 mL) under argon was added palladium on carbon (10% Wt) (37 mg, 0.34 mmol). The reaction mixture was stirred under hydrogen atmosphere at 50° C. for 16 h. The reaction mixture was filtered and the solvent was removed under reduced pressure to afford 2-[2-[2-[5-[5-[tert-butyl(dimethyl)silyl]oxy-1-tetrahydropyran-2-yl-indazol-3-yl]-3-pyridyl]ethoxy]ethoxy] ethanol as a light yellow oil which was used in the next step without further purification.

LCMS method F: [M+H]⁺=542.5, t_{R}=2.82 min

### Preparation of Intermediate 378: [(1R)-2-[2-[2-[5-[5-[tert-butyl(dimethyl)silyl]oxy-1-tetrahydropyran-2-yl-indazol-3-yl]-3-pyridyl]ethoxy]ethoxy]-1-methyl-ethyl]methane sulfonate

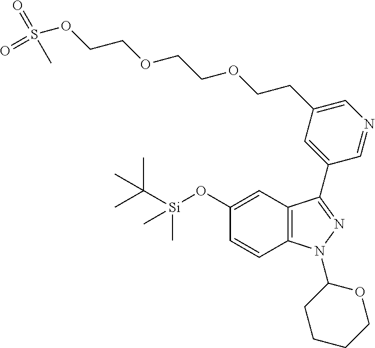 [SMILES: CC(C)(C)[Si](C)(C)Oc1ccc2c(c1)c(-c1cncc(CCOCCOCCOS(C)(=O)=O)c1)nn2C1CCCCO1]

To a suspension 2-[2-[2-[5-[5-[tert-butyl(dimethyl)silyl]oxy-1-tetrahydropyran-2-yl-indazol-3-yl]-3-pyridyl]ethoxy]ethoxy]ethanol (298 mg, 0.55 mmol) in dichloromethane (3 mL) at 0° C. was added triethylamine (115 μL, 0.83 mmol) and methanesulfonyl chloride (51 μL, 0.66 mmol). The reaction mixture was stirred at RT for 1 h then diluted with water. The aqueous layer was extracted with dichloromethane and the combined organic layers were washed with a saturated aqueous ammonium chloride solution, water and brine, dried over anhydrous sodium sulfate, filtered and evaporated under reduced pressure to afford 2-[2-[2-[5-[5-[tert-butyl(dimethyl)silyl]oxy-1-tetrahydropyran-2-yl-indazol-3-yl]-3-pyridyl]ethoxy]ethoxy]ethyl methanesulfonate as a light yellow oil which was used in the next step without further purification.

LCMS method F: [M+H]⁺=620.5, t_{R}=3.08 min

### Preparation of Intermediate 379: 20-(oxan-2-yl)-9,12,15-trioxa-4,20,21-triazatetra cyclo[14.5.2.1^{2,6}.0^{19,22}]tetracosa-1(21),2(24),3,5,16(23),17,19(22)-heptaene

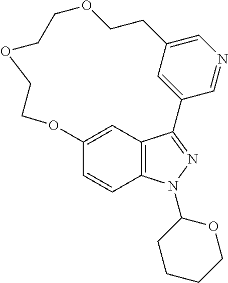 [SMILES: c1ncc2cc1CCOCCOCCOc1ccc3c(c1)c-2nn3C1CCCCO1]

To a suspension of cesium carbonate (694 mg, 2.13 mmol) in anhydrous DMF (70 mL) at 80° C. was added dropwise [(1R)-2-[2-[2-[5-[5-[tert-butyl(dimethyl)silyl]oxy-1-tetrahydropyran-2-yl-indazol-3-yl]-3-pyridyl]ethoxy]ethoxy]-1-methyl-ethyl] methanesulfonate (440 mg, 0.71 mmol) in DMF (60 mL). The reaction mixture was stirred at 80° C. for 30 min. The reaction mixture was filtered and the filtrate was evaporated under reduced pressure. The residue was purified by silica gel column chromatography using cyclohexane/ethyl acetate 50/50 as eluent to afford 20-(oxan-2-yl)-9,12,15-trioxa-4,20,21-triazatetracyclo[14.5.2.1^{2,6}.0^{19,22}]tetracosa-1(21),2(24),3,5,16(23),17,19(22)-heptaene as a yellow oil.

LCMS method F: [M+H]⁺=410.4, t_{R}=1.91 min

### Preparation of Example 68: 9,12,15-trioxa-4,20,21-triazatetracyclo [14.5.2.1^{2,6}.0^{19,22}] tetracosa-1(21),2(24),3,5,16(23),17,19(22)-heptaene

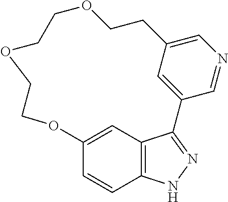 [SMILES: c1ncc2cc1CCOCCOCCOc1ccc3[nH]nc-2c3c1]

To a solution of 20-(oxan-2-yl)-9,12,15-trioxa-4,20,21-triazatetracyclo[14.5.2.1^{2,6}.0^{19,22}] tetracosa-1(21),2(24),3,5,16(23),17,19(22)-heptaene (135 mg, 0.33 mmol) in methanol (6.3 mL) and water (0.9 mL) was added p-toluenesulfonic acid monohydrate (314 mg, 1.65 mmol). The reaction mixture was stirred at 80° C. for 16 h. The reaction mixture was diluted with ethyl acetate and a saturated aqueous NaHCO₃ solution. The aqueous layer was extracted with ethyl acetate. The combined organic layers were washed with brine, dried over anhydrous sodium sulfate, filtered and evaporated under reduced pressure. The resulting solid was recrystallized from acetonitrile, filtered and dried to afford 9,12,15-trioxa-4,20,21-triazatetracyclo [14.5.2.1^{2,6}.0^{19,22}]tetracosa-1(21),2(24),3,5,16(23),17,19(22)-heptaene as a solid.

LCMS method F: [M+H]⁺=326.2, t_{R}=1.54 min

LCMS method G: [M+H]⁺=326.3, t_{R}=1.99 min

1H NMR (400 MHz, d6-DMSO) 13.24 (1H, m), 8.93 (1H, d, J=2.1 Hz), 8.42 (1H, m), 8.25 (1H, s), 7.89 (1H, m), 7.49 (1H, m), 7.06 (1H, dd, J=2.1, 8.9 Hz), 4.29-4.25 (2H, m), 3.80-3.71 (4H, m), 3.64 (4H, s), 2.98 (2H, t, J=4.9 Hz) ppm.

### Example 69: (14S)-14-methyl-9,12,15-trioxa-4,20,21-triazatetracyclo[14.5.2.1^{2,6}.0^{19,22}] tetracosa-1(21),2(24),3,5,16(23),17,19(22)-heptaene

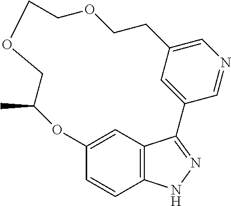 [SMILES: C[C@H]1COCCOCCc2cncc(c2)-c2n[nH]c3ccc(cc23)O1]

Example 69 is prepared according to the synthesis route described in general Scheme D.

### Preparation of Intermediate 380: 3-[2-[2-[(2R)-2-benzyloxypropoxy]ethoxy]ethyl]-5-bromo-pyridine

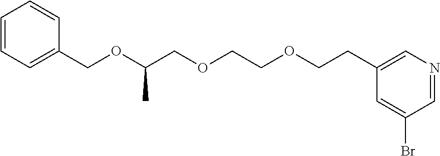 [SMILES: C[C@H](COCCOCCc1cncc(Br)c1)OCc1ccccc1]

To a solution of 2-(5-bromo-3-pyridyl)ethanol (200 mg, 0.99 mmol) in DMF (5 mL) at 0° C. was added sodium hydride (60% dispersion in mineral oil (59 mg, 1.48 mmol) and 2-[(2R)-2-benzyloxypropoxy]ethyl methanesulfonate (intermediate 387) (856 mg, 2.97 mmol) in DMF (3 mL). The reaction mixture was stirred at RT for 16 h. The mixture was diluted with ethyl acetate, washed with water and brine. The organic layer was dried over anhydrous sodium sulfate, filtered and evaporated under reduced pressure. The residue was purified by silica gel column chromatography using cyclohexane/ethyl acetate 100/0 to 80/20 as eluent to afford 3-[2-[2-[(2R)-2-benzyloxypropoxy]ethoxy]ethyl]-5-bromo-pyridine as a colorless oil.

LCMS method F: [M+H]⁺=394.3-396.3, t_{R}=2.77 min

### Preparation of Intermediate 381: [3-[5-[2-[2-[(2R)-2-benzyloxypropoxy]ethoxy] ethyl]-3-pyridyl]-1-tetrahydropyran-2-yl-indazol-5-yl]oxy-tert-butyl-dimethyl-silane

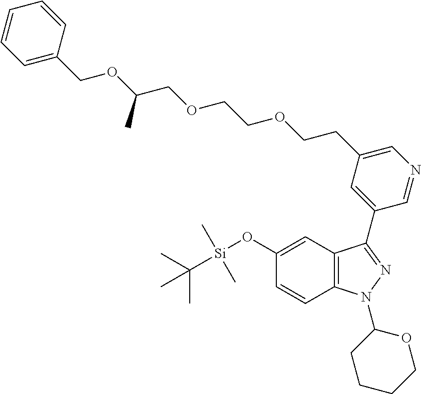 [SMILES: C[C@H](COCCOCCc1cncc(-c2nn(C3CCCCO3)c3ccc(O[Si](C)(C)C(C)(C)C)cc23)c1)OCc1ccccc1]

To a suspension of 3-[2-[2-[(2R)-2-benzyloxypropoxy]ethoxy]ethyl]-5-bromo-pyridine (256 mg, 0.65 mmol) in dioxane (3.2 mL) water (0.3 mL) was added tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (387 mg, 0.84 mmol) and potassium phosphate tribasic (413 mg, 1.95 mmol). The reaction mixture was purged with argon for 15 min then tetrakis(triphenylphosphine) palladium(0) (37 mg, 0.03 mmol) and Xphos (31 mg, 0.06 mmol) were added. The reaction mixtures was stirred under microwave irradiations at 90° C. for 1 h. The reaction mixture was poured into water and extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 80/20 as eluent to afford [3-[5-[2-[2-[(2R)-2-benzyloxypropoxy] ethoxy]ethyl]-3-pyridyl]-1-tetrahydropyran-2-yl-indazol-5-yl]oxy-tert-butyl-dimethyl-silane as a light yellow oil.

LCMS method F: [M+H]⁺=646.6, t_{R}=3.67 min

### Preparation of Intermediate 382: (2R)-1-[2-[2-[5-[5-[tert-butyl(dimethyl)silyl]oxy-1-tetrahydropyran-2-yl-indazol-3-yl]-3-pyridyl]ethoxy]ethoxy]propan-2-ol

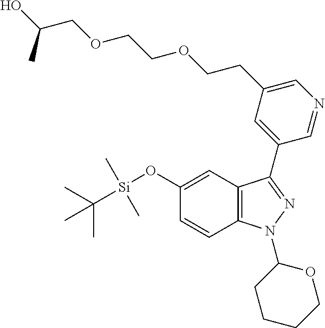 [SMILES: C[C@@H](O)COCCOCCc1cncc(-c2nn(C3CCCCO3)c3ccc(O[Si](C)(C)C(C)(C)C)cc23)c1]

To a solution of [3-[5-[2-[2-[(2R)-2-benzyloxypropoxy]ethoxy]ethyl]-3-pyridyl]-1-tetrahydro pyran-2-yl-indazol-5-yl]oxy-tert-butyl-dimethyl-silane (290 mg, 0.45 mmol) in ethanol (5 mL) under argon was added palladium on carbon (29 mg, 0.27 mmol). The reaction mixture was stirred under hydrogen atmosphere at RT for 120 h. The reaction mixture was filtered and the solvent was removed under reduced pressure to afford (2R)-1-[2-[2-[5-[5-[tert-butyl(dimethyl) silyl]oxy-1-tetrahydropyran-2-yl-indazol-3-yl]-3-pyridyl]ethoxy]ethoxy]propan-2-ol as a light yellow oil which was used in the next step without further purification.

LCMS method F: [M+H]⁺=556.5, t_{R}=2.94 min

### Preparation of Intermediate 383: [(1R)-2-[2-[2-[5-[5-[tert-butyl(dimethyl)silyl]oxy-1-tetrahydropyran-2-yl-indazol-3-yl]-3-pyridyl]ethoxy]ethoxy]-1-methyl-ethyl] methanesulfonate

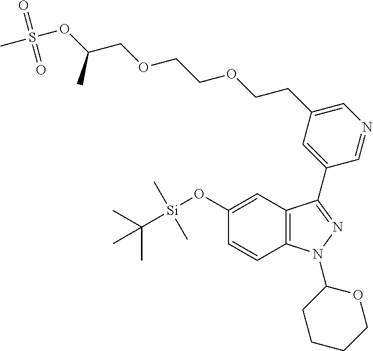 [SMILES: C[C@H](COCCOCCc1cncc(-c2nn(C3CCCCO3)c3ccc(O[Si](C)(C)C(C)(C)C)cc23)c1)OS(C)(=O)=O]

To a suspension (2R)-1-[2-[2-[5-[5-[tert-butyl(dimethyl)silyl]oxy-1-tetrahydropyran-2-yl-indazol-3-yl]-3-pyridyl]ethoxy]ethoxy]propan-2-ol (232 mg, 0.42 mmol) in dichloromethane (20 mL) at 0° C. was added triethylamine (87 μL, 0.63 mmol) and methanesulfonyl chloride (39 μL, 0.50 mmol). The reaction mixture was stirred at RT for 1 h. The reaction mixture was diluted with water and the aqueous layer was extracted with dichloromethane. The combined organic layers were washed with a saturated aqueous ammonium chloride solution, water and brine, dried over anhydrous sodium sulfate, filtered and evaporated under reduced pressure to afford [(1R)-2-[2-[2-[5-[5-[tert-butyl(dimethyl)silyl]oxy-1-tetrahydropyran-2-yl-indazol-3-yl]-3-pyridyl]ethoxy]ethoxy]-1-methyl-ethyl] methanesulfonate as a light yellow oil which was was used in the next step without further purification.

LCMS method F: [M+H]⁺=634.5, t_{R}=3.20 min

### Preparation of Intermediate 384: (14S)-14-methyl-20-(oxan-2-yl)-9,12,15-trioxa-4,20,21-triazatetracyclo[14.5.2.1^{2,6}.0^{19,22}]tetracosa-1(21),2(24),3,5,16(23),17,19(22)-heptaene

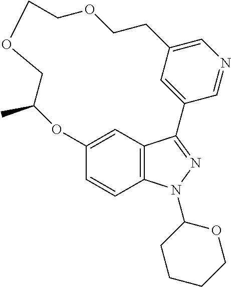 [SMILES: C[C@H]1COCCOCCc2cncc(c2)-c2nn(C3CCCCO3)c3ccc(cc23)O1]

To a suspension of cesium carbonate (523 mg, 1.60 mmol) in anhydrous DMF (5 mL) at 80° C. was added dropwise [(1R)-2-[2-[2-[5-[5-[tert-butyl(dimethyl)silyl]oxy-1-tetrahydropyran-2-yl-indazol-3-yl]-3-pyridyl]ethoxy]ethoxy]-1-methyl-ethyl]methanesulfonate (339 mg, 0.53 mmol) in DMF (5 mL). The reaction mixture was stirred at 80° C. for 30 min. The reaction mixture was filtered and the solvent was evaporated under reduced pressure. The residue was purified by silica gel column chromatography using cyclohexane/ethyl acetate 50/50 as eluent to afford (14S)-14-methyl-20-(oxan-2-yl)-9,12,15-trioxa-4,20,21-triazatetracyclo[14.5.2.1^{2,6}.0^{19,22}]tetracosa-1(21),2(24),3,5,16(23),17,19(22)-heptaene as a yellow foam.

LCMS method F: [M+H]⁺=424.4, t_{R}=2.09 min

### Preparation of Example 69: (14S)-14-methyl-9,12,15-trioxa-4,20,21-triazatetracyclo [14.5.2.1^{2,6}.0^{19,22}]tetracosa-1(21),2(24),3,5,16(23),17,19(22)-heptaene

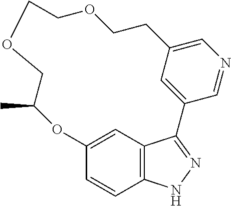 [SMILES: C[C@H]1COCCOCCc2cncc(c2)-c2n[nH]c3ccc(cc23)O1]

To a solution of (14S)-14-methyl-20-(oxan-2-yl)-9,12,15-trioxa-4,20,21-triazatetracyclo [14.5.2.1^{2,6}.0^{19,22}]tetracosa-1(21),2(24),3,5,16(23),17,19(22)-heptaene (28 mg, 0.07 mmol) in methanol (1.3 mL) and water (0.2 mL) was added p-toluenesulfonic acid monohydrate (63 mg, 0.33 mmol). The reaction mixture was stirred at 80° C. for 16 h. The reaction mixture was diluted with ethyl acetate and a saturated aqueous NaHCO₃ solution. The aqueous layer was extracted with ethyl acetate and the combined organic layers were washed with brine, dried over anhydrous sodium sulfate, filtered and evaporated under reduced pressure. The resulting solid was recrystallized from acetonitrile, filtered and dried to afford (14S)-14-methyl-9,12,15-trioxa-4,20,21-triazatetracyclo[14.5.2.1^{2,6}.0^{19,22}]tetracosa-1(21),2(24),3,5,16(23),17,19(22)-heptaene as a solid.

LCMS method F: [M+H]⁺=340.3, t^{R}=1.63 min

LCMS method G: [M+H]⁺=340.3, t^{R}=2.10 min

1H NMR (400 MHz, d6-DMSO) 13.25-13.22 (1H, m), 8.94 (1H, d, J=2.1 Hz), 8.42 (1H, d, J=1.9 Hz), 8.27 (1H, t, J=2.0 Hz), 7.86 (1H, d, J=1.9 Hz), 7.50-7.47 (1H, m), 7.05 (1H, dd, J=2.1, 8.9 Hz), 4.34-4.29 (1H, m), 3.81-3.53 (6H, m), 3.01-2.95 (2H, m), 1.29-1.23 (5H, m) ppm.

### Example 70: (10R)-10-methyl-8,11,14-trioxa-4,5,19,20-tetraazatetracyclo[13.5.2.1^{2,5}.0^{18,21}]tricosa-1(20),2(23),3,15(22),16,18(21)-hexaene

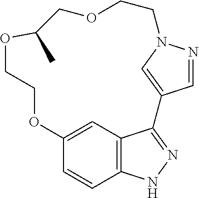 [SMILES: C[C@@H]1COCCn2cc(cn2)-c2n[nH]c3ccc(cc23)OCCO1]

Example 70 is prepared according to the synthesis route described in general Scheme D.

### Preparation of Intermediate 385: 2-[2-[(2R)-2-benzyloxypropoxy]ethoxy]tetra hydropyran

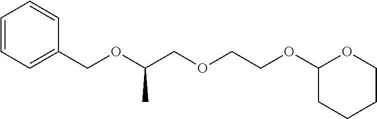 [SMILES: C[C@H](COCCOC1CCCCO1)OCc1ccccc1]

To a solution of (2R)-2-benzyloxypropan-1-ol (intermediate 87) (2 g, 12.04 mmol) in DMF (70 mL) was added portionwise sodium hydride (60% dispersion in mineral oil) (2.408 g, 36.12 mmol). The reaction mixture was stirred at RT for 30 min then a solution of 2-(2-bromo ethoxy)tetrahydropyran (4 mL, 26.50 mmol) in DMF (5 mL) was added. The reaction mixture was stirred at 55° C. for 6 h. The reaction mixture was concentrated then diluted with water. The aqueous layer was extracted with ethyl acetate. The combined organic layers were washed with water, 10% aqueous lithium chloride solution and brine, dried over anhydrous magnesium sulfate, filtered and evaporated under reduced pressure. The residue was purified by silica gel column chromatography using cyclohexane/ethyl acetate 100/0 to 70/30 as eluent to afford 2-[2-[(2R)-2-benzyloxypropoxy]ethoxy]tetrahydropyran as a colorless oil.

LCMS method F: [M+H]⁺=317.2, t_{R}=2.75 min

### Preparation of Intermediate 386: 2-[(2R)-2-benzyloxypropoxy]ethanol

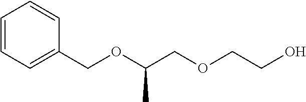

To a solution of 2-[2-[(2R)-2-benzyloxypropoxy]ethoxy]tetrahydropyran (2.202 g, 7.49 mmol) in methanol (35 mL) was added p-toluenesulfonic acid monohydrate (143 mg, 0.75 mmol). The reaction mixture was stirred at RT for 2 h. Water was added and the aqueous layer was extracted with diethylether. The combined organic layers were washed with water, a saturated aqueous NaHCO₃ solution, water and brine, dried over anhydrous magnesium sulfate, filtered and evaporated under reduced pressure to afford 2-[(2R)-2-benzyloxypropoxy]ethanol as a colorless oil which was used in the next step without any further purification.

LCMS method F: [M+H]⁺=211.2, t_{R}=1.95 min

### Preparation of Intermediate 387: 2-[(2R)-2-benzyloxypropoxy]ethyl methane sulfonate

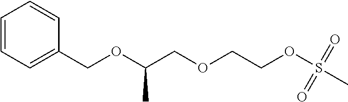 [SMILES: C[C@H](COCCOS(C)(=O)=O)OCc1ccccc1]

To a solution of 2-[(2R)-2-benzyloxypropoxy]ethanol (1.363 g, 6.49 mmol) and triethylamine (1.80 mL, 12.98 mmol) in dichloromethane (60 mL) at 0° C. was added dropwise methanesulfonyl chloride (650 μL, 8.43 mmol). The reaction mixture was stirred at RT for 2 h. The reaction mixture was diluted with water and extracted with dichloromethane. The combined organic layers were washed with brine, dried over anhydrous magnesium sulfate, filtered and evaporated under reduced pressure to afford 2-[(2R)-2-benzyloxypropoxy]ethyl methanesulfonate as a colorless oil which was used in the next step without further purification.

¹H NMR (400 MHz, CDCl₃) 7.39-7.26 (5H, m), 4.65 (1H, J_{AB}=11.0 Hz), 4.57 (1H, J_{AB}=11.0 Hz), 4.40-4.38 (2H, m), 3.79-3.73 (3H, m), 3.60-3.50 (2H, m), 3.02 (3H, s), 1.22 (3H, d, J=6.0 Hz) ppm.

### Preparation of Intermediate 388: 1-[2-[(2R)-2-benzyloxypropoxy]ethyl]-4-bromo-pyrazole

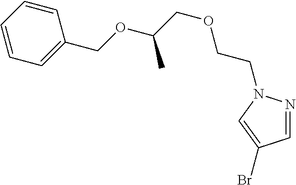 [SMILES: C[C@H](COCCn1cc(Br)cn1)OCc1ccccc1]

A suspension of 4-bromo-1H-pyrazole (1.041 g, 7.14 mmol), 2-[(2R)-2-benzyloxypropoxy] ethyl methanesulfonate (1.87 g, 6.49 mmol), and cesium carbonate (2.739 g, 8.43 mmol) in acetonitrile (28 mL) was stirred at 85° C. for 16 h. The reaction mixture was cooled to RT, filtered over a pad of Celite and washed with ethyl acetate. The filtrate was evaporated under reduced pressure to afford 1-[2-[(2R)-2-benzyloxypropoxy]ethyl]-4-bromo-pyrazole as a yellow oil which was used in the next step without further purification.

LCMS method F: [M+H]⁺=339.2-341.2, t_{R}=2.74 min

### Preparation of Intermediate 389: (2R)-1-[2-(4-bromopyrazol-1-yl)ethoxy]propan-2-ol

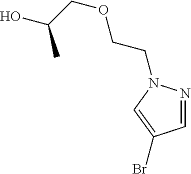

To a solution of 1-[2-[(2R)-2-benzyloxypropoxy]ethyl]-4-bromo-pyrazole (2.2 g, 6.49 mmol) in ethanol (43 mL) was added concentrated hydrochloric acid 37% aqueous solution (32 mL) and the reaction mixture was stirred at 80° C. for 24 h. The reaction mixture was cooled to RT and the solvent was removed under reduced pressure. The residue was dissolved in a saturated aqueous NaHCO₃ solution and extracted with ethyl acetate. The combined organic layers were washed with water and brine, dried over anhydrous magnesium sulfate, filtered and evaporated under reduced pressure. The residue was purified by silica gel column chromatography using dichloromethane/ethyl acetate 100/0 to 80/20 as eluent to afford (2R)-1-[2-(4-bromopyrazol-1-yl)ethoxy]propan-2-ol as a colorless oil.

LCMS method F: [M+H]⁺=249.1-251.1, t_{R}=1.68 min

### Preparation of Intermediate 390: 2-benzyloxyethyl 4-methylbenzenesulfonate

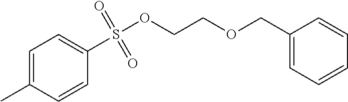 [SMILES: Cc1ccc(S(=O)(=O)OCCOCc2ccccc2)cc1]

To a solution of 2-benzyloxyethanol (1.824 g, 12 mmol) and triethylamine (2.5 mL, 18 mmol) in dichloromethane (38 mL), was added p-toluenesulfonyl chloride (4.56 g, 24 mmol). The reaction mixture was stirred at RT for 16 h. The reaction mixture was washed with a saturated aqueous NaHCO₃ solution, a saturated aqueous ammonium chloride solution and brine. The organic layer was dried over anhydrous magnesium sulfate, filtered and evaporated under reduced pressure. The residue was purified by silica gel column chromatography using cyclohexane/ethyl acetate 99/1 to 80/20 as an eluent to afford 2-benzyloxyethyl 4-methylbenzenesulfonate as a colorless oil.

LCMS method F: [M+H]⁺=307.1, t_{R}=2.83 min

### Preparation of Intermediate 391: 1-[2-[(2R)-2-(2-benzyloxyethoxy)propoxy]ethyl]-4-bromo-pyrazole

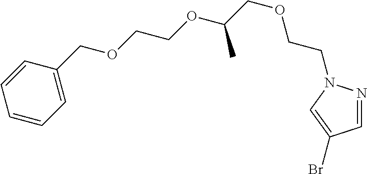 [SMILES: C[C@H](COCCn1cc(Br)cn1)OCCOCc1ccccc1]

To a solution of (2R)-1-[2-(4-bromopyrazol-1-yl)ethoxy]propan-2-ol (340 mg, 1.36 mmol) in THF (10 mL), in a sealed tube under argon was added 2-benzyloxyethyl 4-methylbenzene sulfonate (918 mg, 3 mmol) and potassium hydroxide (266 mg, 4.76 mmol). The reaction mixture stirred at 80° C. for 16 h. The reaction mixture was diluted with dichloromethane and water and the aqueous layer was extracted with dichloromethane. The combined organic layers were washed with brine, dried over anhydrous magnesium sulfate, filtered and evaporated under reduced pressure. The residue was purified by silica gel column chromatography using cyclohexane/ethyl acetate 85/158 as eluent to afford 1-[2-[(2R)-2-(2-benzyloxyethoxy) propoxy]ethyl]-4-bromo-pyrazole as a colorless oil.

LCMS method F: [M+H]⁺=383.2-385.2, t_{R}=2.74 min

### Preparation of Intermediate 392: [3-[1-[2-[(2R)-2-(2-benzyloxyethoxy)propoxy] ethyl]pyrazol-4-yl]-1-tetrahydropyran-2-yl-indazol-5-yl]oxy-tert-butyl-dimethyl-silane

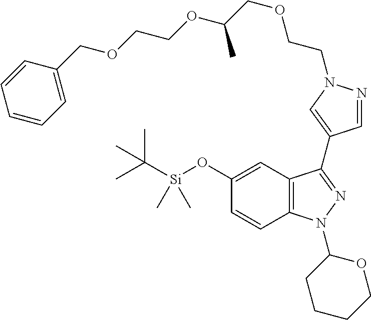 [SMILES: C[C@H](COCCn1cc(-c2nn(C3CCCCO3)c3ccc(O[Si](C)(C)C(C)(C)C)cc23)cn1)OCCOCc1ccccc1]

To a degassed solution of 1-[2-[(2R)-2-(2-benzyloxyethoxy)propoxy]ethyl]-4-bromo-pyrazole (322 mg, 0.84 mmol), tert-butyl-dimethyl-[1-tetrahydropyran-2-yl-3-(4,4,5,5-tetramethyl-1,3,2-dioxaborolan-2-yl)indazol-5-yl]oxy-silane (458 mg, 1 mmol) and potassium phosphate tribasic (534 mg, 2.52 mmol) in dioxane (7 mL) and water (0.3 mL) was added tetrakis(triphenylphosphine)palladium(0) (49 mg, 0.042 mmol) and 2-dicyclohexylphosphino-2′,4′,6′-triisopropylbiphenyl (40 mg, 0.084 mmol). The reaction mixture was stirred at 135° C. for 1 h. The reaction mixture was filtered through a pad of Celite and washed with ethyl acetate. The filtrate was diluted with water and extracted with ethyl acetate. The combined organic layers were washed with water, brine, dried over anhydrous magnesium sulfate, filtered and evaporated under reduced pressure. The residue was purified by silica gel column chromatography using dichloromethane/methanol from 100/0 to 98/2 as eluent to afford [3-[1-[2-[(2R)-2-(2-benzyloxyethoxy)propoxy]ethyl]pyrazol-4-yl]-1-tetrahydropyran-2-yl-indazol-5-yl]oxy-tert-butyl-dimethyl-silane as a yellow oil.

LCMS method F: [M+H]⁺=635.5, t_{R}=3.69 min

### Preparation of Intermediate 393: 2-[(1R)-2-[2-[4-[5-[tert-butyl(dimethyl)silyl]oxy-1-tetrahydropyran-2-yl-indazol-3-yl]pyrazol-1-yl]ethoxy]-1-methyl-ethoxy]ethanol

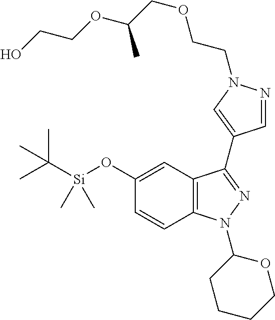 [SMILES: C[C@H](COCCn1cc(-c2nn(C3CCCCO3)c3ccc(O[Si](C)(C)C(C)(C)C)cc23)cn1)OCCO]

To a solution of [3-[1-[2-[(2R)-2-(2-benzyloxyethoxy)propoxy]ethyl]pyrazol-4-yl]-1-tetra hydropyran-2-yl-indazol-5-yl]oxy-tert-butyl-dimethyl-silane (340 mg, 0.54 mmol) in ethanol (8 mL) at RT was added palladium hydroxide on carbon (40 mg). The reaction mixture was stirred under hydrogen atmosphere at RT overnight. The reaction mixture filtered and the filtrate was evaporated under reduced pressure. The residue was purified by silica gel column chromatography using cyclohexane/ethyl acetate 100/0 to 20/80 as eluent to afford 2-[(1R)-2-[2-[4-[5-[tert-butyl(dimethyl)silyl]oxy-1-tetrahydropyran-2-yl-indazol-3-yl]pyrazol-1-yl]ethoxy]-1-methyl-ethoxy]ethanol as a colorless oil.

LCMS method F: [M+H]⁺=545.4, t_{R}=3.20 min

### Preparation of Intermediate 394: 2-[(1R)-2-[2-[4-[5-[tert-butyl(dimethyl)silyl]oxy-1-tetrahydropyran-2-yl-indazol-3-yl]pyrazol-1-yl]ethoxy]-1-methyl-ethoxy]ethyl methanesulfonate

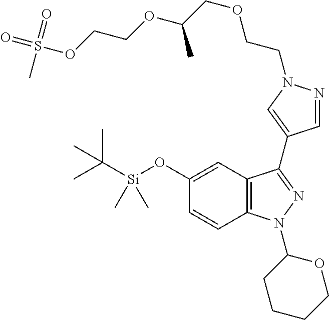 [SMILES: C[C@H](COCCn1cc(-c2nn(C3CCCCO3)c3ccc(O[Si](C)(C)C(C)(C)C)cc23)cn1)OCCOS(C)(=O)=O]

To a solution of 2-[(1R)-2-[2-[4-[5-[tert-butyl(dimethyl)silyl]oxy-1-tetrahydropyran-2-yl-indazol-3-yl]pyrazol-1-yl]ethoxy]-1-methyl-ethoxy]ethanol (242 mg, 0.45 mmol) and triethylamine (120 μL, 0.90 mmol) in dichloromethane (5 mL) at 0° C. was added dropwise methanesulfonyl chloride (100 μL, 0.58 mmol). The reaction mixture was stirred at RT for 2 h.

The mixture was diluted with water and extracted with dichloromethane. The combined organic layers were washed with brine, dried over anhydrous magnesium sulfate, filtered and evaporated under reduced pressure to afford 2-[(1R)-2-[2-[4-[5-[tert-butyl(dimethyl)silyl]oxy-1-tetrahydropyran-2-yl-indazol-3-yl]pyrazol-1-yl]ethoxy]-1-methyl-ethoxy]ethylmethane sulfonate as a colorless oil which was used in the next step without further purification.

LCMS method F: [M+H]⁺=623.4, t_{R}=3.34 min

### Preparation of Intermediate 395: (10R)-10-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene

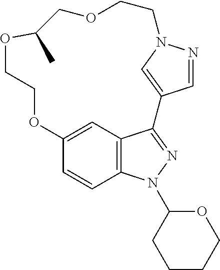 [SMILES: C[C@@H]1COCCn2cc(cn2)-c2nn(C3CCCCO3)c3ccc(cc23)OCCO1]

To a suspension of cesium carbonate (390 mg, 1.20 mmol) in anhydrous DMF (20 mL) at 60° C. was added dropwise 2-[(1R)-2-[2-[4-[5-[tert-butyl(dimethyl)silyl]oxy-1-tetrahydropyran-2-yl-indazol-3-yl]pyrazol-1-yl]ethoxy]-1-methyl-ethoxy]ethylmethanesulfonate (249 mg, 0.40 mmol) in DMF (10 mL). The reaction mixture was stirred at 60° C. for 16 h. The reaction mixture was cooled to RT, filtered over a pad of Celite and washed with ethyl acetate. The filtrate was evaporated under reduced pressure. The residue was purified by silica gel column chromatography using dichloromethane/methanol 100/0 to 95/5 as eluent to afford (10R)-10-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene as a colorless oil.

LCMS method F: [M+H]⁺=413.3, t_{R}=2.54 min

### Preparation of Example 70: (10R)-10-methyl-8,11,14-trioxa-4,5,19,20-tetraazatetra cyclo[13.5.2.1^{2,5}.0^{18,21}]tricosa-1(20),2(23),3,15(22),16,18(21)-hexaene

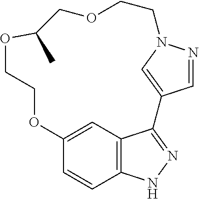 [SMILES: C[C@@H]1COCCn2cc(cn2)-c2n[nH]c3ccc(cc23)OCCO1]

To a solution of (10R)-10-methyl-19-(oxan-2-yl)-8,11,14-trioxa-4,5,19,20-tetraazatetracyclo [13.5.2.1^{2,5}.0^{18,21}]tricosa-1(20),2(23),3,15(22),16,18(21)-hexaene (102 mg, 0.25 mmol) in methanol (4.3 mL) and water (0.7 mL) was added p-toluenesulfonic acid monohydrate (237 mg, 1.25 mmol). The reaction mixture was stirred at 65° C. for 6 h. The reaction mixture was diluted with dichloromethane and with a saturated aqueous NaHCO₃ solution. The aqueous layer was extracted with dichloromethane. The combined organic layers were washed with brine, dried over anhydrous sodium sulfate, filtered and evaporated under reduced pressure. The residue was crystallized with dichloromethane, filtered and dried to afford (10R)-10-methyl-8,11,14-trioxa-4,5,19,20-tetraazatetracyclo[13.5.2.1^{2,5}.0^{18,21}]tricosa-1(20),2(23),3, 15(22),16,18(21)-hexaene as a solid.

LCMS method F: [M+H]⁺=329.2, t_{R}=1.99 min

LCMS method G: [M+H]⁺=329.3, t_{R}=2.01 min

1H NMR (400 MHz, d6-DMSO) 12.74 (1H, s), 8.49 (1H, s), 7.82-7.79 (2H, m), 7.41-7.37 (1H, m), 6.99 (1H, dd, J=2.2, 8.8 Hz), 4.41-4.30 (4H, m), 3.99-3.92 (1H, m), 3.82-3.70 (3H, m), 3.67-3.60 (2H, m), 3.49 (1H, dd, J=5.5, 10.1 Hz), 1.16 (3H, d, J=6.3 Hz) ppm.

### Example 71: (13R)-13-methyl-8,11,14-trioxa-4,5,19,20,23-pentaazatetracyclo[13.5.2.1^{2,5}.0^{18,21}]tricosa-1(20),2(23),3,15(22),16,18(21)-hexaene

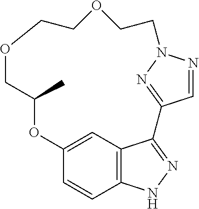 [SMILES: C[C@@H]1COCCOCCn2ncc(n2)-c2n[nH]c3ccc(cc23)O1]

Example 71 is prepared according to the synthesis route described in general Scheme D.

### Preparation of Intermediate 396: 4,5-dibromo-2-(2-tetrahydropyran-2-yloxyethyl) triazole

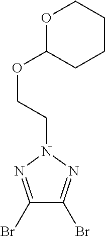 [SMILES: Brc1nn(CCOC2CCCCO2)nc1Br]

To a stirred solution of 4,5-dibromo-2H-triazole (3.4 g, 15 mmol) in DMF (100 mL) at −10° C. was added potassium carbonate (4.14 g, 30 mmol) and the suspension was stirred for 15 min. A solution of 2-(2-bromoethoxy)tetrahydropyran (3.44 g, 16.5 mmol) in DMF (10 mL) was added dropwise. The reaction mixture was stirred at RT overnight. The reaction mixture was diluted with water and extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 90/10 to 80/20 as eluent to afford 4,5-dibromo-2-(2-tetrahydropyran-2-yloxyethyl)triazole as a colorless liquid.

LCMS method F: [M+H]⁺=354.1-356.1-358.1, t_{R}=2.69 min

### Preparation of Intermediate 397: 4-bromo-2-(2-tetrahydropyran-2-yloxyethyl) triazole

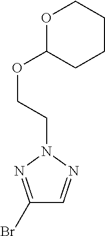 [SMILES: Brc1cnn(CCOC2CCCCO2)n1]

To a solution of 4,5-dibromo-2-(2-tetrahydropyran-2-yloxyethyl) triazole (1.9 g, 5.35 mmol) in THF (50 mL) at −20° C. was added dropwise iPrMgCl (2M solution in THF) (3.2 mL, 6.42 mmol). The reaction mixture was stirred at RT overnight. The reaction mixture was quenched with a saturated aqueous ammonium chloride solution and extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 90/10 as eluent to afford 4-bromo-2-(2-tetrahydropyran-2-yloxyethyl)triazole as a colorless liquid.

LCMS method F: [M+H]⁺=276.1-278.1, t_{R}=2.34 min

### Preparation of Intermediate 398: 2-(4-bromotriazol-2-yl)ethanol

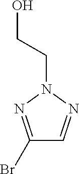

To a solution of 4-bromo-2-(2-tetrahydropyran-2-yloxyethyl) triazole (1.66 g, 6 mmol) in a mixture of methanol (71 mL) and water (10 mL) was added p-toluenesulfonic acid monohydrate (3.42 g, 18 mmol). The reaction mixture was stirred at RT overnight. The solvent was partially removed under reduced pressure and the solution was basified with a saturated aqueous NaHCO₃ solution and extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 70/30 as eluent to afford 2-(4-bromotriazol-2-yl)ethanol as a colorless liquid.

LCMS method F: [M+H]⁺=192.1-194.1, t_{R}=1.21 min

### Preparation of Intermediate 399: 2-[2-[(2S)-2-benzyloxypropoxy]ethoxy]tetra hydropyran

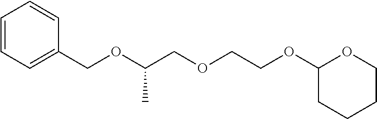 [SMILES: C[C@@H](COCCOC1CCCCO1)OCc1ccccc1]

To a suspension of sodium hydride (60% dispersion in mineral oil) (930 mg, 23.17 mmol) in anhydrous DMF (10 mL) at 0° C. was added dropwise 2-(tetrahydro-2H-pyran-2-yloxy)ethanol (2.71 g, 18.54 mmol) in DMF (5 mL). After stirring for 20 min at 0° C., (2S)-2-(benzyloxy)propyl-4-methylbenzene-1-sulfonate (intermediate 105) (4.95 g, 15.45 mmol) in DMF (5 mL) was added dropwise. The reaction mixture was stirred at 70° C. for 1.5 h. The reaction mixture was quenched by addition of water and concentrated under reduced pressure. The residue was diluted with brine and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 90/10 as eluent to afford 2-[2-[(2S)-2-benzyloxypropoxy]ethoxy]tetrahydropyran as a colorless oil.

LCMS method F: [M+Na]⁺=317.3, t_{R}=2.77 min

### Preparation of Intermediate 400: 2-[(2S)-2-benzyloxypropoxy]ethanol

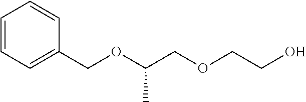

To a solution of 2-[2-[(2S)-2-benzyloxypropoxy]ethoxy]tetrahydropyran (3 g, 10.19 mmol) in methanol (35 mL) and water (5 mL) was added p-toluenesulfonic acid monohydrate (9.69 g, 50.95 mmol). The reaction mixture was stirred at RT for 96 h. The reaction mixture was concentrated under reduced pressure and the residue was neutralized by slow addition of a saturated aqueous NaHCO₃ solution. Ethyl acetate was added, the phases were separated and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 70/30 as eluent to afford 2-[(2S)-2-benzyloxypropoxy]ethanol as a colorless oil.

LCMS method F: [M+H]⁺=211.2, t_{R}=1.96 min

### Preparation of Intermediate 401: 2-[(2S)-2-benzyloxypropoxy]ethyl 4-methylbenzene sulfonate

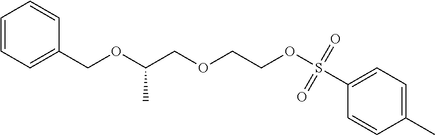 [SMILES: Cc1ccc(S(=O)(=O)OCCOC[C@H](C)OCc2ccccc2)cc1]

To a solution of 2-[(2S)-2-benzyloxypropoxy]ethanol (631 mg, 3 mmol) and triethylamine (836 μL, 6 mmol) in dichloromethane (10 mL) was added portionwise at 0° C. p-toluenesulfonyl chloride (744 mg, 3.9 mmol). The reaction mixture was stirred at RT overnight. The reaction mixture was diluted with a saturated aqueous ammonium chloride solution and extracted with dichloromethane. The combined organic layers were washed with brine, dried over anhydrous sodium sulfate, filtered and evaporated under reduced pressure. The residue was purified by silica gel column chromatography using cyclohexane/ethyl acetate 100/0 to 70/30 as eluent to afford 2-[(2S)-2-benzyloxypropoxy]ethyl 4-methylbenzenesulfonate as a yellow oil.

LCMS method F: [M+H]⁺=365.2, t_{R}=2.94 min

### Preparation of Intermediate 402: 2-[2-[2-[(2S)-2-benzyloxypropoxy]ethoxy]ethyl]-4-bromo-triazole

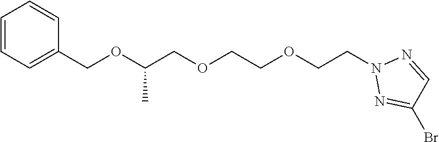 [SMILES: C[C@@H](COCCOCCn1ncc(Br)n1)OCc1ccccc1]

To a suspension of sodium hydride (60% dispersion in mineral oil) (125 mg, 3.13 mmol) in anhydrous DMF (6 mL) at 0° C. was added dropwise 2-(4-bromotriazol-2-yl)ethanol (400 mg, 2.08 mmol) in DMF (1 mL). After stirring for 20 min at 0° C., 2-[(2S)-2-benzyloxypropoxy]ethyl 4-methylbenzenesulfonate (911 mg, 2.50 mmol) in DMF (1 mL) was added dropwise. The reaction mixture was stirred at 70° C. for 3 h. The reaction mixture was quenched by addition of water and was concentrated under reduced pressure. The residue was diluted with brine and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 90/10 as eluent. to afford 2-[2-[2-[(2S)-2-benzyloxypropoxy]ethoxy]ethyl]-4-bromo-triazole as a colorless oil.

LCMS method F: [M+H]⁺=384.2-386.2, t_{R}=2.81 min

### Preparation of Intermediate 403: [3-[2-[2-[2-[(2S)-2-benzyloxypropoxy]ethoxy]ethyl] triazol-4-yl]-1-tetrahydropyran-2-yl-indazol-5-yl]oxy-tert-butyl-dimethyl-silane

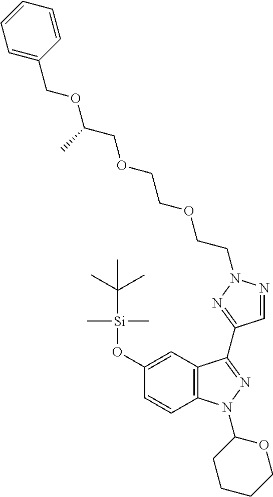 [SMILES: C[C@@H](COCCOCCn1ncc(-c2nn(C3CCCCO3)c3ccc(O[Si](C)(C)C(C)(C)C)cc23)n1)OCc1ccccc1]

To a solution of tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (730 mg, 1.59 mmol) in dioxane (10 mL) and water (1 mL) at RT was added 2-[2-[2-[(2S)-2-benzyloxypropoxy]ethoxy]ethyl]-4-bromo-triazole (510 mg, 1.33 mmol), potassium phosphate tribasic (845 mg, 3.98 mmol), XPhos (63 mg, 0.13 mmol) and tetrakis(triphenylphosphine)palladium(0) (77 mg, 0.07 mmol). The reaction mixture was stirred at 90° C. under microwave irradiations for 1.5 h. The reaction mixture was diluted with brine and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 80/20 as eluent to afford [3-[2-[2-[2-[(2S)-2-benzyloxypropoxy]ethoxy]ethyl]triazol-4-yl]-1-tetrahydropyran-2-yl-indazol-5-yl]oxy-tert-butyl-dimethyl-silane as a yellow oil.

LCMS method F: [M+H]⁺=636.4, t_{R}=3.82 min

### Preparation of Intermediate 404: (2S)-1-[2-[2-[4-[5-[tert-butyl(dimethyl)silyl]oxy-1-tetrahydropyran-2-yl-indazol-3-yl]triazol-2-yl]ethoxy]ethoxy]propan-2-ol

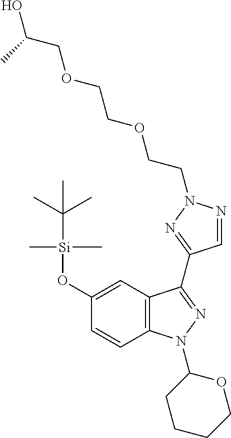 [SMILES: C[C@H](O)COCCOCCn1ncc(-c2nn(C3CCCCO3)c3ccc(O[Si](C)(C)C(C)(C)C)cc23)n1]

To a solution of [3-[2-[2-[2-[(2S)-2-benzyloxypropoxy]ethoxy]ethyl]triazol-4-yl]-1-tetrahydro pyran-2-yl-indazol-5-yl]oxy-tert-butyl-dimethyl-silane (730 mg, 1.15 mmol) in dichloro methane (23 mL) and pH 7 phosphate buffer (1.17 mL) at RT was added 2,3-dichloro-5,6-dicyano-p-benzoquinone (651 mg, 2.87 mmol). The reaction mixture was stirred at RT for 24 h. The reaction mixture was diluted with a saturated aqueous NaHCO₃ solution and extracted with ethyl acetate. The combined organic layers were washed with brine, dried over anhydrous sodium sulfate, filtered and evaporated under reduced pressure. The residue was purified by silica gel column chromatography using dichloromethane/ethyl acetate 100/0 to 40/60 as eluent to afford (2S)-1-[2-[2-[4-[5-[tert-butyl(dimethyl)silyl]oxy-1-tetrahydropyran-2-yl-indazol-3-yl]triazol-2-yl]ethoxy]ethoxy]propan-2-ol as a colorless oil.

LCMS method F: [M+H]⁺=546.4, t_{R}=3.35 min

### Preparation of Intermediate 405: [(1S)-2-[2-[2-[4-[5-[tert-butyl(dimethyl)silyl]oxy-1-tetrahydropyran-2-yl-indazol-3-yl]triazol-2-yl]ethoxy]ethoxy]-1-methyl-ethyl] methanesulfonate

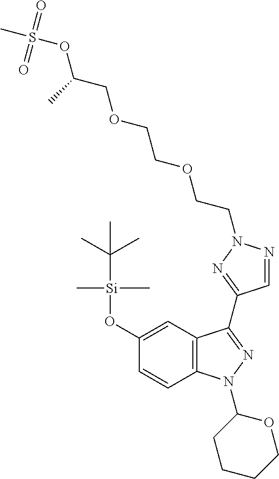 [SMILES: C[C@@H](COCCOCCn1ncc(-c2nn(C3CCCCO3)c3ccc(O[Si](C)(C)C(C)(C)C)cc23)n1)OS(C)(=O)=O]

To a solution of (2S)-1-[2-[2-[4-[5-[tert-butyl(dimethyl)silyl]oxy-1-tetrahydropyran-2-yl-indazol-3-yl]triazol-2-yl]ethoxy]ethoxy]propan-2-ol (340 mg, 0.62 mmol) and triethylamine (174 μL, 1.25 mmol) in dichloromethane (8 mL) at 0° C. was added methanesulfonyl chloride (72 μL, 0.93 mmol). The reaction mixture was stirred at RT for 3 h. The reaction mixture was diluted with brine and extracted with dichloromethane. The combined organic layers were dried over anhydrous sodium sulfate, filtered and evaporated under reduced pressure to afford [(1S)-2-[2-[2-[4-[5-[tert-butyl(dimethyl)silyl]oxy-1-tetrahydropyran-2-yl-indazol-3-yl]triazol-2-yl] ethoxy]ethoxy]-1-methyl-ethyl]methanesulfonate as a yellow oil which was used in the next step without further purification.

LCMS method F: [M+H]⁺=624.5, t_{R}=3.50 min

### Preparation of Intermediate 406: (13R)-13-methyl-19-(oxan-2-yl)-8,11,14-trioxa-4,5,19,20,23-pentaazatetracyclo[13.5.2.1^{2,5}.0^{18,21}]tricosa-1(20),2(23),3,15(22),16, 18(21)-hexaene

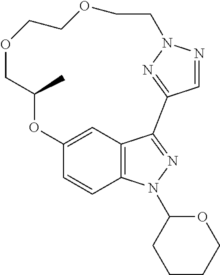 [SMILES: C[C@@H]1COCCOCCn2ncc(n2)-c2nn(C3CCCCO3)c3ccc(cc23)O1]

To a suspension of cesium carbonate (606 mg, 1.86 mmol) in anhydrous DMF (105 mL) at 60° C. was added dropwise [(1S)-2-[2-[2-[4-[5-[tert-butyl(dimethyl)silyl]oxy-1-tetrahydro pyran-2-yl-indazol-3-yl]triazol-2-yl]ethoxy]ethoxy]-1-methyl-ethyl]methanesulfonate (387 mg, 0.62 mmol) in DMF (105 mL). The reaction mixture was stirred at 60° C. overnight. The reaction mixture was concentrated under reduced pressure, diluted with brine and extracted with ethyl acetate. The combined organic layers were dried over anhydrous sodium sulfate, filtered and evaporated under reduced pressure. The residue was purified by silica gel column chromatography using dichloromethane/ethyl acetate 100/0 to 70/30 as eluent to afford (13R)-13-methyl-19-(oxan-2-yl)-8,11,14-trioxa-4,5,19,20,23-pentaazatetracyclo[13.5.2.1^{2,5}.0^{18,21}] tricosa-1(20),2(23),3,15(22),16,18(21)-hexaene as a white solid.

LCMS method F: [M+H]⁺=414.4, t_{R}=2.60 min

### Preparation of Example 71: (13R)-13-methyl-8,11,14-trioxa-4,5,19,20,23-pentaazatetracyclo[13.5.2.1^{2,5}.0^{18,21}]tricosa-1(20),2(23),3,15(22),16,18(21)-hexaene

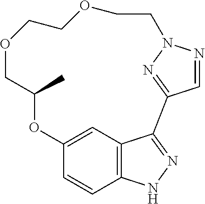 [SMILES: C[C@@H]1COCCOCCn2ncc(n2)-c2n[nH]c3ccc(cc23)O1]

To a solution of (13R)-13-methyl-19-(oxan-2-yl)-8,11,14-trioxa-4,5,19,20,23-pentaazatetra cyclo[13.5.2.1^{2,5}.0^{18,21}]tricosa-1(20),2(23),3,15(22),16,18(21)-hexaene (170 mg, 0.41 mmol) in methanol (14 mL) and water (2 mL) was added p-toluenesulfonic acid monohydrate (391 mg, 2.06 mmol). The reaction mixture was stirred at 60° C. overnight. The reaction mixture was concentrated under reduced pressure and the residue was neutralized by addition of a saturated aqueous NaHCO₃ solution. The mixture was diluted with ethyl acetate. The phases were separated and the aqueous layer was extracted with ethyl acetate. The combined organic layers were washed with brine, dried over anhydrous sodium sulfate, filtered and evaporated under reduced pressure. The residue was recrystallized from acetonitrile, filtered and dried under reduced pressure to afford (13R)-13-methyl-8,11,14-trioxa-4,5,19,20,23-pentaazatetracyclo [13.5.2.1^{2,5}.0^{18,21}]tricosa-1(20),2(23),3,15(22),16,18(21)-hexaene as a solid.

LCMS method F: [M+H]⁺=330.3, t_{R}=1.95 min

LCMS method G: [M+H]⁺=330.3, t_{R}=1.94 min

1H NMR (400 MHz, d6-DMSO) 13.08 (1H, s), 8.21 (1H, d, J=2.1 Hz), 8.08 (1H, s), 7.44-7.42 (1H, m), 7.01 (1H, dd, J=2.5, 8.9 Hz), 4.73-4.68 (2H, m), 4.34-4.29 (1H, m), 4.16-4.10 (1H, m), 3.87-3.59 (5H, m), 3.56-3.42 (2H, m), 1.34 (3H, d, J=6.5 Hz) ppm.

### Example 72: (13S)-13-methyl-8,11,14-trioxa-5,19,20-triazatetracyclo[13.5.2.1^{2,5}.0^{18,21}] tricosa-1(20),2(23),3,15(22),16,18(21)-hexaene-4-carbonitrile

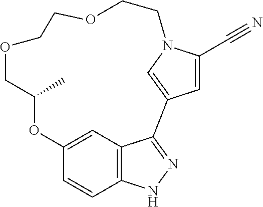 [SMILES: C[C@H]1COCCOCCn2cc(cc2C#N)-c2n[nH]c3ccc(cc23)O1]

Example 72 is prepared according to the synthesis route described in general Scheme D.

### Preparation of Intermediate 407: 4-bromo-1-(2-((tert-butyldimethylsilyl)oxy)ethyl)-pyrrole-2-carbonitrile

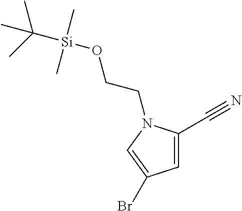 [SMILES: CC(C)(C)[Si](C)(C)OCCn1cc(Br)cc1C#N]

To a solution of 4-bromo-1H-pyrrole-2-carbonitrile (2.02 g, 11.81 mmol) in DMA (50 mL) was added cesium carbonate (5.77 g, 17.72 mmol) and 2-bromoethoxy-tert-butyl dimethylsilane (3.02 mL, 14.17 mmol). The reaction mixture was stirred at RT for 15 h. The reaction mixture was diluted with ethyl acetate and washed with brine. The organic layer was dried over anhydrous magnesium sulfate, filtered and evaporated under reduced pressure. The residue was purified by silica gel column chromatography using heptane/ethyl acetate 100/0 to 90/10 as eluent to afford 4-bromo-1-(2-((tert-butyldimethylsilyl)oxy)ethyl)-pyrrole-2-carbonitrile as a colorless oil.

LCMS method B: [M+H]⁺=328.8, t_{R}=1.293 min

### Preparation of Intermediate 408: 4-bromo-1-(2-hydroxyethyl)-pyrrole-2-carbonitrile

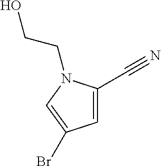 [SMILES: N#Cc1cc(Br)cn1CCO]

To a solution of 4-bromo-1-(2-((tert-butyldimethylsilyl)oxy)ethyl)-pyrrole-2-carbonitrile (3.89 g, 11.81 mmol) in THF (60 mL) at 0° C. was added TBAF (1M solution in THF) (17.71 mL, 17.71 mmol). The reaction mixture was stirred at RT for 15 h. The reaction mixture was diluted with ethyl acetate and washed with a saturated aqueous NaHCO₃ solution and brine. The organic layer was dried over anhydrous magnesium sulfate, filtered and evaporated under reduced pressure. The residue was purified by silica gel column chromatography using heptane/ethyl acetate 100/0 to 50/50/as eluent to afford 4-bromo-1-(2-hydroxyethyl)-pyrrole-2-carbonitrile as a colorless oil.

LCMS method B: [M+H]⁺=214.9, t_{R}=0.473 min

### Preparation of Intermediate 409: 4-bromo-1-[2-(2-tetrahydropyran-2-yloxyethoxy) ethyl]pyrrole-2-carbonitrile

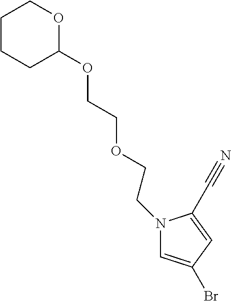 [SMILES: N#Cc1cc(Br)cn1CCOCCOC1CCCCO1]

To a solution of 4-bromo-1-(2-hydroxyethyl)pyrrole-2-carbonitrile (600 mg, 2.79 mmol) in DMF (18 mL) was added sodium hydride (60% dispersion in mineral oil) (558 mg, 8.37 mmol) in one portion. The reaction mixture was stirred at RT for 30 min then 2-(2-bromoethoxy) tetrahydropyran (920 μL, 6.14 mmol) was added dropwise. The reaction mixture was stirred at RT for 2 h. The reaction mixture was concentrated under reduced pressure and diluted with water and ethyl acetate. The phases were separated and the combined organic layers were washed with water, 10% aqueous lithium chloride solution and brine, dried over anhydrous magnesium sulfate, filtered and evaporated under reduced pressure. The residue was purified by silica gel column chromatography using cyclohexane/ethyl acetate from 100/0 to 70/30 as eluent to afford 4-bromo-1-[2-(2-tetrahydropyran-2-yloxyethoxy)ethyl]pyrrole-2-carbonitrile as a colorless oil.

LCMS method F: [M+Na]⁺=365.1-367.1, t_{R}=2.67 min

### Preparation of Intermediate 410: 4-bromo-1-[2-(2-hydroxyethoxy)ethyl]pyrrole-2-carbonitrile

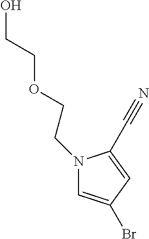 [SMILES: N#Cc1cc(Br)cn1CCOCCO]

To a solution of 4-bromo-1-[2-(2-tetrahydropyran-2-yloxyethoxy)ethyl]pyrrole-2-carbonitrile (654 mg, 1.91 mmol) in methanol (9 mL) was added p-toluenesulfonic acid monohydrate (36 mg, 0.19 mmol). The reaction mixture was stirred at RT for 2 h. Water was added and the mixture was extracted with diethylether. The combined organic layers were washed with water, saturated aqueous NaHCO₃ solution, water and brine, dried over anhydrous magnesium sulfate, filtered and evaporated under reduced pressure to afford 4-bromo-1-[2-(2-hydroxyethoxy)ethyl]pyrrole-2-carbonitrile as a colorless oil which was used in the next step without further purification.

LCMS method F: [M+H]⁺=257.1-259.1, t_{R}=1.96 min

### Preparation of Intermediate 411: 2-[2-(4-bromo-2-cyano-pyrrol-1-yl)ethoxy]ethyl 4-methylbenzenesulfonate

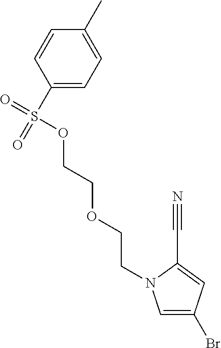 [SMILES: Cc1ccc(S(=O)(=O)OCCOCCn2cc(Br)cc2C#N)cc1]

To a solution of 4-bromo-1-[2-(2-hydroxyethoxy)ethyl]pyrrole-2-carbonitrile (250 mg, 0.96 mmol) and triethylamine (200 μL, 1.44 mmol) in dichloromethane (3 mL) was added p-toluenesulfonyl chloride (365 mg, 1.92 mmol) in one portion. The reaction mixture was stirred at RT for 16 h. The reaction mixture was washed with a saturated aqueous NaHCO₃ solution. The organic layer was dried over anhydrous magnesium sulfate, filtered and evaporated under reduced pressure to afford 2-[2-(4-bromo-2-cyano-pyrrol-1-yl)ethoxy]ethyl 4-methylbenzene sulfonate as a colorless oil which was used in the next step without further purification.

LCMS method F: [M+H]⁺=413.1-415.1, t_{R}=2.84 min

### Preparation of Intermediate 412: 1-[2-[2-[(2R)-2-benzyloxypropoxy]ethoxy]ethyl]-4-bromo-pyrrole-2-carbonitrile

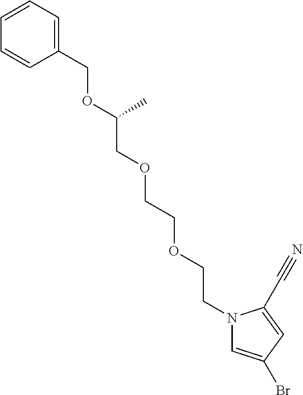 [SMILES: C[C@H](COCCOCCn1cc(Br)cc1C#N)OCc1ccccc1]

To a stirring solution of (2R)-2-benzyloxypropan-1-ol (intermediate 87) (318 mg, 1.92 mmol) in DMF (10 mL) was added sodium hydride (60% dispersion in mineral oil) (192 mg, 2.88 mmol) in one portion. The reaction mixture was stirred at RT for 30 min and a solution of 2-[2-(4-bromo-2-cyano-pyrrol-1-yl)ethoxy]ethyl-4-methylbenzenesulfonate (396 mg, 0.96 mmol) in DMF (5 mL) was added. The reaction mixture was stirred at RT for 2 h. The reaction mixture was poured into a saturated aqueous solution of ammonium chloride at 0° C. and the organic layer was extracted with ethyl acetate. The combined organic layers were washed with water, 10% aqueous lithium chloride solution and brine, dried over anhydrous magnesium sulfate, filtered and evaporated under reduced pressure. The residue was purified by silica gel column chromatography using cyclohexane/ethyl acetate from 100/0 to 80/20 as eluent to afford 1-[2-[2-[(2R)-2-benzyloxypropoxy]ethoxy]ethyl]-4-bromo-pyrrole-2-carbonitrile as a slightly yellow oil.

LCMS method F: [M+H]⁺=407.1-409.1, t_{R}=3.02 min

### Preparation of Intermediate 413: 1-[2-[2-[(2R)-2-benzyloxypropoxy]ethoxy]ethyl]-4-[5-[tert-butyl(dimethyl)silyl]oxy-1-tetrahydropyran-2-yl-indazol-3-yl]pyrrole-2-carbonitrile

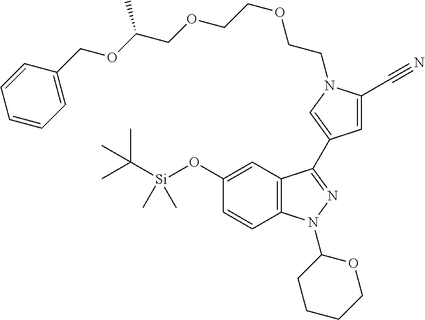 [SMILES: C[C@H](COCCOCCn1cc(-c2nn(C3CCCCO3)c3ccc(O[Si](C)(C)C(C)(C)C)cc23)cc1C#N)OCc1ccccc1]

To a degassed solution of 1-[2-[2-[(2R)-2-benzyloxypropoxy]ethoxy]ethyl]-4-bromo-pyrrole-2-carbonitrile (230 mg, 0.57 mmol), tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (311 mg, 0.68 mmol) and potassium phosphate tribasic (362 mg, 1.71 mmol) in dioxane (5 mL) and water (0.25 mL) was added tetrakis(triphenylphosphine)palladium(0) (32 mg, 0.028 mmol) and 2-dicyclohexylphosphino-2′,4′,6′-triisopropylbiphenyl (27 mg, 0.057 mmol). The reaction mixture was stirred at 110° C. for 1 h. The reaction mixture was filtered through a pad of Celite and washed with ethyl acetate. The filtrate was diluted with water and extracted with ethyl acetate. The combined organic layers were washed with water, brine, dried over anhydrous magnesium sulfate, filtered and evaporated under reduced pressure. The residue was purified by silica gel column chromatography using dichloromethane/methanol 100/0 to 98/2 as eluent to afford 1-[2-[2-[(2R)-2-benzyloxypropoxy]ethoxy]ethyl]-4-[5-[tert-butyl(dimethyl)silyl] oxy-1-tetrahydropyran-2-yl-indazol-3-yl]pyrrole-2-carbonitrile as a colorless oil.

LCMS method F: [M+H]⁺=659.5, t_{R}=3.88 min

### Preparation of Intermediate 414: 4-[5-[tert-butyl(dimethyl)silyl]oxy-1-tetrahydropyran-2-yl-indazol-3-yl]-1-[2-[2-[(2R)-2-hydroxypropoxy]ethoxy]ethyl] pyrrole-2-carbonitrile

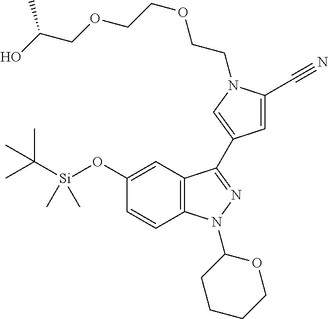 [SMILES: C[C@@H](O)COCCOCCn1cc(-c2nn(C3CCCCO3)c3ccc(O[Si](C)(C)C(C)(C)C)cc23)cc1C#N]

To a solution of 1-[2-[2-[(2R)-2-benzyloxypropoxy]ethoxy]ethyl]-4-[5-[tert-butyl(dimethyl) silyl]oxy-1-tetrahydropyran-2-yl-indazol-3-yl]pyrrole-2-carbonitrile (300 mg, 0.45 mmol) in ethyl acetate (7.2 mL) at RT was added palladium on carbon 10 wt. % (40 mg). The reaction mixture was stirred under hydrogen atmosphere at RT for 40 h. The reaction mixture was filtered and the filtrate was concentrated under reduced pressure. The residue was purified by silica gel column chromatography using cyclohexane/ethyl acetate, 100/0 to 50/50 as eluent to afford 4-[5-[tert-butyl(dimethyl)silyl]oxy-1-tetrahydropyran-2-yl-indazol-3-yl]-1-[2-[2-[(2R)-2-hydroxypropoxy]ethoxy]ethyl]pyrrole-2-carbonitrile as a colorless oil.

LCMS method F: [M+H]⁺=569.4, t_{R}=3.43 min

### Preparation of Intermediate 415: [(1R)-2-[2-[2-[4-[5-[tert-butyl(dimethyl)silyl]oxy-1-tetrahydropyran-2-yl-indazol-3-yl]-2-cyano-pyrrol-1-yl]ethoxy]ethoxy]-1-methyl-ethyl]methanesulfonate

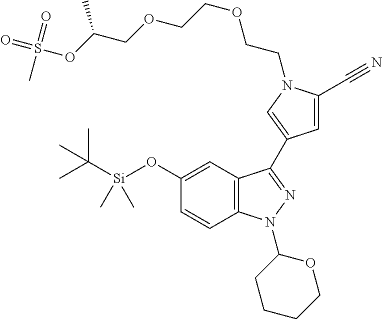 [SMILES: C[C@H](COCCOCCn1cc(-c2nn(C3CCCCO3)c3ccc(O[Si](C)(C)C(C)(C)C)cc23)cc1C#N)OS(C)(=O)=O]

To a solution of 4-[5-[tert-butyl(dimethyl)silyl]oxy-1-tetrahydropyran-2-yl-indazol-3-yl]-1-[2-[2-[(2R)-2-hydroxypropoxy]ethoxy]ethyl]pyrrole-2-carbonitrile (120 mg, 0.21 mmol) and triethylamine (58 μL, 0.42 mmol) in dichloromethane (2 mL) at 0° C., was added dropwise methanesulfonyl chloride (20 μL, 0.27 mmol). The reaction mixture was stirred at RT for 2 h. The mixture was diluted with water and extracted with dichloromethane. The combined organic layers were washed with brine, dried over anhydrous magnesium sulfate, filtered and evaporated under reduced pressure to afford [(1R)-2-[2-[2-[4-[5-[tert-butyl(dimethyl)silyl]oxy-1-tetrahydropyran-2-yl-indazol-3-yl]-2-cyano-pyrrol-1-yl]ethoxy]ethoxy]-1-methyl-ethyl] methanesulfonate as a colorless oil which was used in the next step without further purification.

LCMS method F: [M+H]⁺=647.4, t_{R}=3.55 min

### Preparation of Intermediate 416: (13S)-13-methyl-19-(oxan-2-yl)-8,11,14-trioxa-5,19,20-triazatetracyclo[13.5.2.1^{2,5}.0^{18,21}]tricosa-1(20),2(23),3,15(22),16,18(21)-hexaene-4-carbonitrile

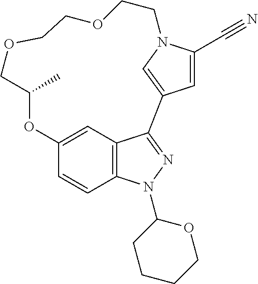 [SMILES: C[C@H]1COCCOCCn2cc(cc2C#N)-c2nn(C3CCCCO3)c3ccc(cc23)O1]

To a suspension of cesium carbonate (205 mg, 0.63 mmol) in anhydrous DMF (10 mL) at 60° C. was added dropwise [(1R)-2-[2-[2-[4-[5-[tert-butyl(dimethyl)silyl]oxy-1-tetrahydropyran-2-yl-indazol-3-yl]-2-cyano-pyrrol-1-yl]ethoxy]ethoxy]-1-methyl-ethyl] methanesulfonate (135 mg, 0.21 mmol) in DMF (5 mL). The reaction mixture was stirred at 65° C. for 16 h. The reaction mixture was cooled to RT, filtered over a pad of Celite and washed with ethyl acetate. The filtrate was concentrated under reduced pressure. The residue was purified by silica gel column chromatography using the eluent dichloromethane/methanol 100/0 to 95/5 as eluent to afford (13S)-13-methyl-19-(oxan-2-yl)-8,11,14-trioxa-5,19,20-triazatetracyclo[13.5.2.1^{2,5}.0^{18,21}] tricosa-1(20),2(23),3,15(22),16,18(21)-hexaene-4-carbonitrile as a white solid.

LCMS method F: [M+H]⁺=437.4, t_{R}=2.96 min

### Preparation of Example 72: (13S)-13-methyl-8,11,14-trioxa-5,19,20-triazatetracyclo [13.5.2.1^{2,5}.0^{18,21}]tricosa-1(20),2(23),3,15(22),16,18(21)-hexaene-4-carbonitrile

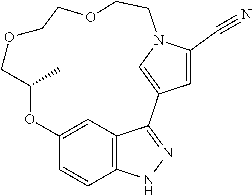 [SMILES: C[C@H]1COCCOCCn2cc(cc2C#N)-c2n[nH]c3ccc(cc23)O1]

To a solution of (13S)-13-methyl-19-(oxan-2-yl)-8,11,14-trioxa-5,19,20-triazatetracyclo [13.5.2.1^{2,5}.0^{18,21}]tricosa-1(20),2(23),3,15(22),16,18(21)-hexaene-4-carbonitrile (87 mg, 0.20 mmol) in methanol (3.4 mL) and water (0.5 mL) was added p-toluenesulfonic acid monohydrate (190 mg, 1.00 mmol). The reaction mixture was stirred at 65° C. for 6 h. The reaction mixture was diluted with dichloromethane and with a saturated aqueous NaHCO₃ solution. The aqueous layer was extracted with dichloromethane. The combined organic layers were washed with brine, dried over anhydrous sodium sulfate, filtered and evaporated under reduced pressure. The residue was purified by preparative TLC on silica gel using dichloromethane/methanol 95/5 as eluent to afford (13S)-13-methyl-8,11,14-trioxa-5,19,20-triazatetracyclo[13.5.2.1^{2,5}.0^{18,21}] tricosa-1(20),2(23),3,15(22),16,18(21)-hexaene-4-carbonitrile as a solid.

LCMS method F: [M+H]⁺=353.3, t_{R}=2.32 min

LCMS method G: [M+H]⁺=353.3, t_{R}=2.31 min

1H NMR (400 MHz, CDCl₃) 8.04 (1H, s), (1H, d, J=1.7 Hz), 7.98 (1H, d, J=2.1 Hz), 7.35 (1H, d, J=8.9 Hz), 7.32 (1H, d, J=1.7 Hz), 7.11 (1H, dd, J=2.3, 8.9 Hz), 4.52-4.45 (1H, m), 4.37-4.33 (2H, m), 4.06-4.01 (1H, m), 3.89-3.66 (6H, m), 3.58 (1H, dd, J=2.7, 10.2 Hz), 1.44 (3H, d, J=6.6 Hz) ppm.

### Example 73: 8,11,14-trioxa-4,5,19,20,22-pentaazatetracyclo[13.5.2.1^{2,5}.0^{18,21}]tricosa-1(20),2(23),3,15(22), 16,18(21)-hexaene

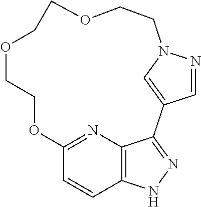 [SMILES: c1nn2cc1-c1n[nH]c3ccc(nc13)OCCOCCOCC2]

Example 73 is prepared according to the synthesis route described in general Scheme A.

### Preparation of Intermediate 417: 5-methoxy-1H-pyrazolo[4,3-b]pyridine

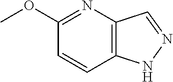

To a solution of 6-methoxy-2-methyl-pyridin-3-amine (4 g, 28.95 mmol) in acetic acid (40 mL) at 0° C. was added dropwise a solution of sodium nitrite (2.99 g, 43.42 mmol) in water (8 mL). The reaction mixture was stirred at 0° C. for 1 h. The pH of the mixture was adjusted from 3 to 7 at 0° C. with a 32% aqueous sodium hydroxide solution. Ethyl acetate was added and the resulting precipitate was filtered. The filtrate was extracted with ethyl acetate and the combined organic layers were washed with brine, dried over anhydrous sodium sulfate, filtered and evaporated under reduced pressure to afford 5-methoxy-1H-pyrazolo[4,3-b]pyridine as a brown solid which was used in the next step without further purification.

LCMS method F: [M+H]⁺=150.1, t_{R}=1.44 min

### Preparation of Intermediate 418: 3-iodo-5-methoxy-1H-pyrazolo[4,3-b]pyridine

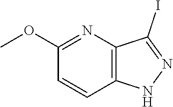

To a solution of 5-methoxy-1H-pyrazolo[4,3-b]pyridine (3.56 g, 23.89 mmol) in acetonitrile (48 mL) was added N-iodosuccinimide (5.38 g, 23.89 mmol). The reaction mixture was stirred at RT for 2.5 h. The reaction mixture was diluted with a saturated sodium thiosulfate solution and ethyl acetate. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 60/40 as eluent. to afford 3-iodo-5-methoxy-1H-pyrazolo [4,3-b]pyridine as an orange solid.

LCMS method F: [M+H]⁺=276.0, t_{R}=2.10 min

### Preparation of Intermediate 419: 3-iodo-5-methoxy-1-tetrahydropyran-2-yl-pyrazolo [4,3-b]pyridine

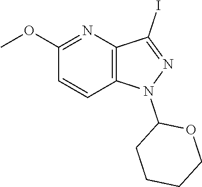 [SMILES: COc1ccc2c(n1)c(I)nn2C1CCCCO1]

To a solution of 3-iodo-5-methoxy-1H-pyrazolo[4,3-b]pyridine (1.31 g, 4.76 mmol) in dichloromethane (8.6 mL) and few drops of THF were added p-toluenesulfonic acid monohydrate (452 mg, 2.38 mmol) and 3,4-dihydro-2H-pyran (870 μL, 9.52 mmol). The reaction mixture was stirred at RT for 2 h. The reaction mixture was diluted with a saturated aqueous NaHCO₃ solution and dichloromethane. The aqueous layer was extracted with dichloromethane and the combined organic layers were washed with brine, dried over anhydrous sodium sulfate, filtered and evaporated under reduced pressure to afford 3-iodo-5-methoxy-1-tetrahydropyran-2-yl-pyrazolo[4,3-b]pyridine as an yellow/orange oil which was used in the next step without further purification.

LCMS method F: [M+H]⁺=360.1, t_{R}=2.82 min

### Preparation of Intermediate 420: 3-iodo-1-tetrahydropyran-2-yl-pyrazolo[4,3-b] pyridin-5-ol

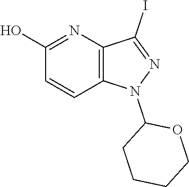 [SMILES: Oc1ccc2c(n1)c(I)nn2C1CCCCO1]

To a degassed solution of 3-iodo-5-methoxy-1-tetrahydropyran-2-yl-pyrazolo[4,3-b]pyridine (1.94 g, 4.42 mmol) in acetonitrile (3 mL) was added sodium iodide (1.98 g, 13.26 mmol). Trimethylsilyl chloride (1.2 mL, 9.54 mmol) was added at 0° C. The reaction mixture was stirred at 80° C. for 1 h. The reaction mixture was diluted with water and ethyl acetate. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/(ethyl acetate/ethanol 3/1) 100/0 to 30/70 as eluent to afford 3-iodo-1-tetrahydropyran-2-yl-pyrazolo [4,3-b]pyridin-5-ol as a yellow oil.

LCMS method F: [M+H]⁺=346.0, t_{R}=1.85 min

### Preparation of Intermediate 421: 2-[2-[2-(2-benzyloxyethoxy)ethoxy]ethoxy]tetra hydropyran

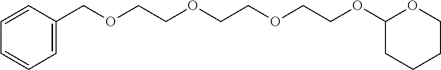 [SMILES: c1ccc(COCCOCCOCCOC2CCCCO2)cc1]

To a solution of 2-(2-benzyloxyethoxy)ethanol (3.4 g, 17.3 mmol) in DMF (50 mL) was added portionwise sodium hydride (60% dispersion in mineral oil (2.08 g, 52 mmol). The reaction mixture was stirred at RT for 30 min. A solution of 2-(2-bromoethoxy)tetrahydropyran (7.9 mL, 52 mmol) in DMF (50 mL) was added. The reaction mixture was stirred at 55° C. for 12 h. The reaction mixture was concentrated and diluted with water and ethyl acetate. The combined organic layers were washed with water, 10% lithium chloride aqueous solution and brine, dried over anhydrous magnesium sulfate, filtered and evaporated under reduced pressure. The residue was purified by silica gel column chromatography using cyclohexane/ethyl acetate 100/0 to 50/50 as eluent to afford 2-[2-[2-(2-benzyloxyethoxy)ethoxy]ethoxy]tetrahydropyran as a yellow oil.

LCMS method F: [M+H]⁺=347.3, t_{R}=2.46 min

### Preparation of Intermediate 422: 2-[2-(2-benzyloxyethoxy)ethoxy]ethanol

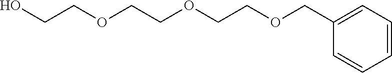 [SMILES: OCCOCCOCCOCc1ccccc1]

To a solution of 2-[2-[2-(2-benzyloxyethoxy)ethoxy]ethoxy]tetrahydropyran (4.64 g, 14.3 mmol) in methanol (80 mL) was added p-toluenesulfonic acid monohydrate (266 mg, 1.4 mmol). The reaction mixture was stirred at RT for 1 h. The solvent was partially removed under reduced pressure. The reaction was quenched with a saturated aqueous NaHCO₃ solution until basic pH then ethyl acetate was added. The aqueous layer was extracted with ethyl acetate and the combined organic layers were washed with brine, dried over anhydrous sodium sulfate, filtered and evaporated under reduced pressure to afford 2-[2-(2-benzyloxyethoxy) ethoxy]ethanol as a yellow oil which was used in the next step without further purification.

LCMS method F: [M+H]⁺=241.2, t_{R}=1.86 min

### Preparation of Intermediate 423: 2-[2-(2-benzyloxyethoxy)ethoxy]ethylmethane sulfonate

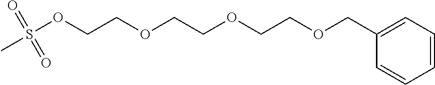 [SMILES: CS(=O)(=O)OCCOCCOCCOCc1ccccc1]

To a solution of 2-[2-(2-benzyloxyethoxy)ethoxy]ethanol (360 mg, 1.5 mmol) in dichloromethane (9 mL) at 0° C. was added triethylamine (420 μL, 3 mmol) and methane sulfonyl chloride (150 μL, 1.95 mmol). The reaction mixture was stirred at RT overnight. Water and dichloromethane were added. The organic layer was washed with a saturated aqueous NaHCO₃ solution and aqueous ammonium chloride solution. The organic layer was dried over anhydrous sodium sulfate, filtered and evaporated under reduced pressure to afford 2-[2-(2-benzyloxyethoxy)ethoxy]ethyl methanesulfonate as an yellow liquid which was used in the next step without further purification.

LCMS method F: [M+H]⁺=319, t_{R}=2.24 min

### Preparation of Intermediate 424: 5-[2-[2-(2-benzyloxyethoxy)ethoxy]ethoxy]-3-iodo-1-tetrahydropyran-2-yl-pyrazolo[4,3-b]pyridine

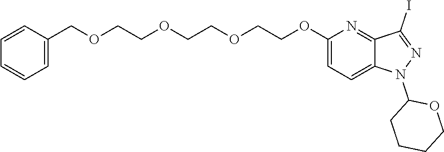 [SMILES: Ic1nn(C2CCCCO2)c2ccc(OCCOCCOCCOCc3ccccc3)nc12]

To a solution of 3-iodo-1-tetrahydropyran-2-yl-pyrazolo[4,3-b]pyridin-5-ol (intermediate 420) (363 mg, 1.05 mmol) in dry acetonitrile (7 mL) was added cesium carbonate (682 mg, 2.1 mmol) and 2-[2-(2-benzyloxyethoxy)ethoxy]ethyl methanesulfonate (370 mg, 1.16 mmol) in acetonitrile (2 mL). The reaction mixture was stirred at 75° C. for 3 h. The reaction was diluted with water and ethyl acetate. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford 5-[2-[2-(2-benzyloxyethoxy)ethoxy]ethoxy]-3-iodo-1-tetrahydropyran-2-yl-pyrazolo[4,3-b]pyridine as a pale yellow oil.

LCMS method F: [M+H]⁺=568.3, t_{R}=3.14 min

### Preparation of Intermediate 425: trimethyl-[2-[[4-(4,4,5,5-tetramethyl-1,3,2-dioxa borolan-2-yl)pyrazol-1-yl]methoxy]ethyl]silane and [1-(2-trimethylsilylethoxymethyl) pyrazol-4-yl]boronic acid

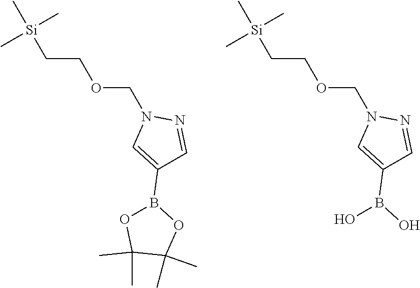

To a solution of 4-(4,4,5,5-tetramethyl-1,3,2-dioxaborolan-2-yl)-1H-pyrazole (3 g, 15.46 mmol) in NMP (23 mL) at RT was added potassium carbonate (4.27 g, 30.92 mmol) and 2-(chloromethoxy)ethyl-trimethyl-silane (2.87 mL, 16.24 mmol). The reaction mixture was stirred at RT overnight. Additional 2-(trimethylsilyl)ethoxymethyl chloride (140 μL, 0.77 mmol) was added and the reaction mixture was stirred at RT for 24 h. The reaction mixture was diluted with ethyl acetate and filtered. The filtrate was washed with water and brine. The organic layer was dried over anhydrous sodium sulfate, filtered and evaporated under reduced pressure to afford a mixture of trimethyl-[2-[[4-(4,4,5,5-tetramethyl-1,3,2-dioxaborolan-2-yl) pyrazol-1-yl]methoxy]ethyl]silane and [1-(2-trimethylsilylethoxymethyl)pyrazol-4-yl]boronic acid as a colorless viscous liquid which was used in the next step without further purification.

LCMS method F: [M+H]⁺=325.3, t_{R}=3.07 min and [M+H]⁺=243.2, t_{R}=2.10 min

### Preparation of Intermediate 426: 2-[[4-[5-[2-[2-(2-benzyloxyethoxy)ethoxy]ethoxy]-1-tetrahydropyran-2-yl-pyrazolo[4,3-b]pyridin-3-yl]pyrazol-1-yl]methoxy]ethyl-trimethyl-silane

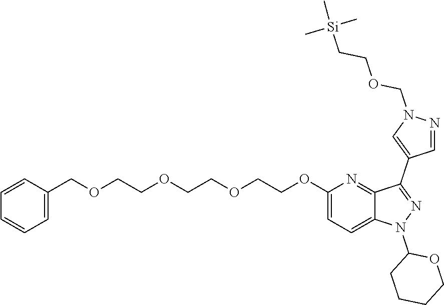 [SMILES: C[Si](C)(C)CCOCn1cc(-c2nn(C3CCCCO3)c3ccc(OCCOCCOCCOCc4ccccc4)nc23)cn1]

To a degassed solution of 5-[2-[2-(2-benzyloxyethoxy)ethoxy]ethoxy]-3-iodo-1-tetrahydro pyran-2-yl-pyrazolo[4,3-b]pyridine (311 mg, 0.55 mmol) in dioxane (3.1 mL) and water (1.1 mL) at RT was added a mixture of trimethyl-[2-[[4-(4,4,5,5-tetramethyl-1,3,2-dioxaborolan-2-yl)pyrazol-1-yl]methoxy]ethyl]silane and [1-(2-trimethylsilylethoxymethyl)pyrazol-4-yl] boronic acid (267 mg, 0.83 mmol), potassium phosphate tribasic (350 mg, 1.65 mmol), XPhos (26 mg, 0.055 mmol) and tetrakis(triphenylphosphine)palladium(0) (32 mg, 0.028 mmol). The reaction mixture was stirred at 100° C. under microwave irradiations for 1 h. The reaction mixture was diluted with water and extracted with ethyl acetate. The combined organic layers were washed with brine, dried over anhydrous sodium sulfate and evaporated under reduced pressure. The residue was purified by silica gel column chromatography using cyclohexane/ethyl acetate 100/0 to 60/40 as eluent to afford 2-[[4-[5-[2-[2-(2-benzyloxyethoxy)ethoxy]ethoxy]-1-tetrahydropyran-2-yl-pyrazolo[4,3-b]pyridin-3-yl]pyrazol-1-yl]methoxy]ethyl-trimethyl-silane as a pale yellow oil.

LCMS method F: [M+H]⁺=638.5, t_{R}=3.47 min

### Preparation of Intermediate 427: 2-[2-[2-[1-tetrahydropyran-2-yl-3-[1-(2-trimethyl silylethoxymethyl)pyrazol-4-yl]pyrazolo[4,3-b]pyridin-5-yl]oxyethoxy]ethoxy] ethanol

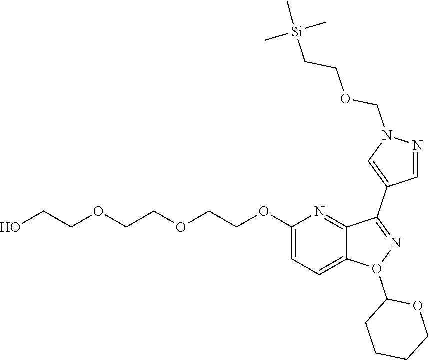

To a solution of 2-[[4-[5-[2-[2-(2-benzyloxyethoxy)ethoxy]ethoxy]-1-tetrahydropyran-2-yl-pyrazolo[4,3-b]pyridin-3-yl]pyrazol-1-yl]methoxy]ethyl-trimethyl-silane (245 mg, 0.38 mmol) in ethyl acetate (8 mL) at RT was added palladium hydroxide on carbon (24 mg). The reaction mixture was stirred under hydrogen atmosphere at RT for 24 h. The reaction mixture was filtered and the filtrate was concentrated under reduced pressure to afford 2-[2-[2-[1-tetrahydro pyran-2-yl-3-[1-(2-trimethylsilylethoxymethyl)pyrazol-4-yl]pyrazolo[4,3-b]pyridin-5-yl]oxy ethoxy]ethoxy]ethanol as a colorless viscous oil which was used in the next step without further purification.

LCMS method F: [M+H]⁺=548.4, t_{R}=2.85 min

### Preparation of Intermediate 428: 2-[2-[2-[3-(1H-pyrazol-4-yl)-1-tetrahydropyran-2-yl-pyrazolo[4,3-b]pyridin-5-yl]oxyethoxy]ethoxy]ethanol

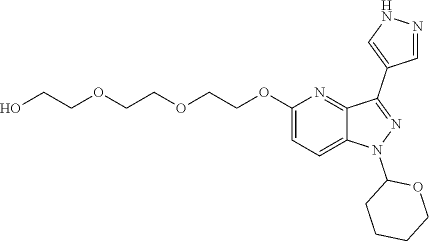 [SMILES: OCCOCCOCCOc1ccc2c(n1)c(-c1cn[nH]c1)nn2C1CCCCO1]

To a solution of 2-[2-[2-[1-tetrahydropyran-2-yl-3-[1-(2-trimethylsilylethoxymethyl)pyrazol-4-yl]pyrazolo[4,3-b]pyridin-5-yl]oxyethoxy]ethoxy]ethanol (201 mg, 0.37 mmol) in THF (4 mL) at RT was added TBAF (1M solution in THF) (740 μL, 0.74 mmol). The reaction mixture was stirred at 60° C. for 27 h. Additional TBAF (1M solution in THF) (100 μL, 0.1 mmol) was added and the reaction mixture was stirred at 60° C. for 1 h. Additional TBAF (1M solution in THF) (370 μL, 0.37 mmol) was added and the mixture was stirred at 60° C. overnight. The reaction mixture was quenched with a saturated ammonium chloride solution and extracted with ethyl acetate. The combined organic layers was washed with brine, dried over anhydrous sodium sulfate and evaporated under reduced pressure to give 2-[2-[2-[3-(1H-pyrazol-4-yl)-1-tetrahydropyran-2-yl-pyrazolo[4,3-b]pyridin-5-yl]oxyethoxy]ethoxy]ethanol as an orange viscous oil which was used in the next step without further purification.

LCMS method F: [M+H]⁺=418.2, t_{R}=2.01 min

### Preparation of Intermediate 429: 2-[2-[2-[3-(1H-pyrazol-4-yl)-1-tetrahydropyran-2-yl-pyrazolo[4,3-b]pyridin-5-yl]oxyethoxy]ethoxy]ethyl 4-methylbenzenesulfonate

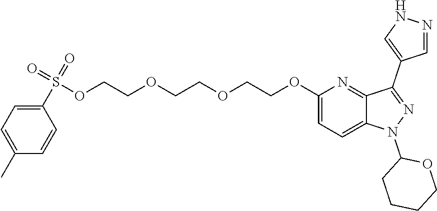 [SMILES: Cc1ccc(S(=O)(=O)OCCOCCOCCOc2ccc3c(n2)c(-c2cn[nH]c2)nn3C2CCCCO2)cc1]

To a solution of 2-[2-[2-[3-(1H-pyrazol-4-yl)-1-tetrahydropyran-2-yl-pyrazolo[4,3-b]pyridin-5-yl]oxyethoxy]ethoxy]ethanol (190 mg, 0.37 mmol) in pyridine (2 mL) at −5° C. was added dropwise p-toluenesulfonyl chloride (78 mg, 0.41 mmol) in pyridine (1 mL). The reaction mixture was slowly warmed to RT and stirred overnight. Additional p-toluenesulfonyl chloride (21 mg, 0.11 mmol) in pyridine (0.8 mL) was added and the reaction mixture was stirred at RT for 4 h. Additional p-toluenesulfonyl chloride (25 mg, 0.13 mmol) in pyridine (1 mL) was added and the reaction mixture was stirred at RT for 3 h. Additional p-toluenesulfonyl chloride (21 mg, 0.11 mmol) in pyridine (0.8 mL) was added and the reaction mixture was stirred at RT overnight. Additional p-toluenesulfonyl chloride (14 mg, 0.074 mmol) was added and the reaction mixture was stirred at RT for 2 h. Additional p-toluenesulfonyl chloride (21 mg, 0.11 mmol) was added and the mixture was stirred at RT for 2 h. The reaction mixture was diluted with a saturated NaHCO₃ solution and extracted with ethyl acetate. The combined organic layers were washed with brine, dried over anhydrous sodium sulfate and evaporated under reduced pressure to afford 2-[2-[2-[3-(1H-pyrazol-4-yl)-1-tetrahydropyran-2-yl-pyrazolo[4,3-b]pyridin-5-yl]oxyethoxy]ethoxy] ethyl 4-methylbenzenesulfonate as a brown paste which was used in the next step without further purification.

LCMS method F: [M+H]⁺=572.3, t_{R}=2.74 min

### Preparation of Intermediate 430: 19-(oxan-2-yl)-8,11,14-trioxa-4,5,19,20,22-pentaaza tetracyclo[13.5.2.1^{2,5}.0^{18,21}]tricosa-1(20),2(23),3,15(22),16,18(21)-hexaene

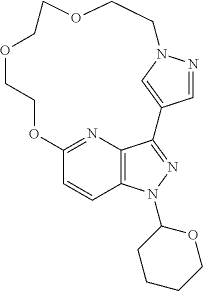 [SMILES: c1nn2cc1-c1nn(C3CCCCO3)c3ccc(nc13)OCCOCCOCC2]

To a solution of sodium hydride (60% dispersion in mineral oil) (40 mg, 0.99 mmol) in dry DMF (140 mL) at RT was added dropwise 2-[2-[2-[3-(1H-pyrazol-4-yl)-1-tetrahydropyran-2-yl-pyrazolo[4,3-b]pyridin-5-yl]oxyethoxy]ethoxy]ethyl 4-methylbenzenesulfonate (188 mg, 0.33 mmol) in dry DMF (140 mL). The reaction mixture was stirred at RT overnight. The reaction mixture was diluted with dry DMF (100 mL) and additional sodium hydride (60% dispersion in mineral oil) (132 mg, 3.3 mmol) was added. The reaction mixture was stirred at RT overnight. The reaction mixture was quenched with methanol and the solvent was removed under reduced pressure. The residue was purified by silica gel column chromatography using dichloromethane/methanol 100/0 to 95/5 as eluent. The resulting oil was purified by preparative TLC eluting with dichloromethane/methanol 95/5 as eluent to afford 19-(oxan-2-yl)-8,11,14-trioxa-4,5,19,20,22-pentaazatetracyclo[13.5.2.1^{2,5}.0^{18,21}]tricosa-1(20),2(23),3,15(22),16, 18(21)-hexaene as a white solid.

LCMS method F: [M+H]⁺=400.4, t_{R}=2.35 min

### Preparation of Example 73: 8,11,14-trioxa-4,5,19,20,22-pentaazatetracyclo[13.5.2.1^{2,5}.0^{18,21}]tricosa-1(20),2(23),3,15(22), 16,18(21)-hexaene

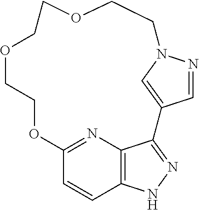 [SMILES: c1nn2cc1-c1n[nH]c3ccc(nc13)OCCOCCOCC2]

To a solution of 19-(oxan-2-yl)-8,11,14-trioxa-4,5,19,20,22-pentaazatetracyclo[13.5.2.1^{2,5}.0^{18,21}]tricosa-1(20),2(23),3,15(22),16,18(21)-hexaene (26 mg, 0.065 mmol) in methanol (1.11 mL) and water (0.19 mL) was added p-toluenesulfonic acid monohydrate (62 mg, 0.326 mmol). The reaction mixture was stirred at 65° C. overnight. The reaction mixture was diluted with dichloromethane and saturated aqueous NaHCO₃ solution. The aqueous layer was extracted with dichloromethane. The combined organic layers were washed with brine, dried over anhydrous sodium sulfate, filtered and evaporated under reduced pressure. The residue was triturated in diisopropyl ether, filtered, washed with diisopropyl ether and dried to afford 8,11,14-trioxa-4,5,19,20,22-pentaazatetracyclo[13.5.2.1^{2,5}.0^{18,21}]tricosa-1(20),2(23),3,15(22), 16,18(21)-hexaene as a powder.

LCMS method F: [M+H]⁺=316.3, t_{R}=1.77 min

LCMS method G: [M+H]⁺=316.2, t_{R}=1.77 min

1H NMR (400 MHz, d6-DMSO) 12.92 (1H, s), 8.94 (1H, s), 7.93 (1H, d, J=9 Hz), 7.88 (1H, s), 6.86 (1H, d, J=9 Hz), 4.57 (2H, t, J=6.6 Hz), 4.38 (2H, m), 3.87 (2H, t, J=6.7 Hz), 3.81 (2H, m), 3.64 (4H, m) ppm.

### Example 74: 9,13-dioxa-4,5,18,19,22-pentaazatetracyclo[12.5.2.1^{2,5}.0^{17,20}]docosa-1(19), 2(22),3,14(21),15,17(20)-hexaene

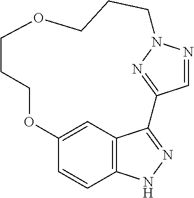 [SMILES: c1cc2[nH]nc3c2cc1OCCCOCCCn1ncc-3n1]

Example 74 is prepared according to the synthesis route described in general Scheme D.

### Preparation of Intermediate 431: 3-benzyloxypropyl 4-methylbenzenesulfonate

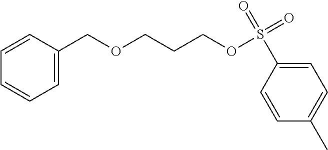 [SMILES: Cc1ccc(S(=O)(=O)OCCCOCc2ccccc2)cc1]

To a solution of 3-benzyloxy-1-propanol (900 μL, 5.68 mmol) and triethylamine (1.6 mL, 11.37 mmol) in dry dichloromethane (16.5 mL) at 0° C. was added p-toluenesulfonyl chloride (1.62 g, 8.53 mmol). The reaction mixture was stirred at 0° C. for 10 min then RT for 16 h. The reaction mixture was quenched with a saturated aqueous NaHCO₃ solution and extracted with dichloromethane. The combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford 3-benzyloxypropyl 4-methylbenzenesulfonate as a pale yellow oil.

LCMS method F: [M+H]⁺=321.2, t_{R}=2.95 min

### Preparation of Intermediate 432: tert-butyl-[3-(4,5-dibromotriazol-2-yl)propoxy]-dimethyl-silane

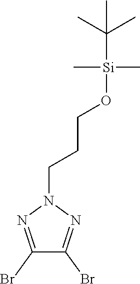 [SMILES: CC(C)(C)[Si](C)(C)OCCCn1nc(Br)c(Br)n1]

To a solution of 4,5-dibromo-2H-triazole (2.983 g, 13.15 mmol) in dry DMF (100 mL) at −10° C. was added potassium carbonate (3.635 g, 26.3 mmol). The suspension was stirred at −10° C. for 20 min then (3-bromopropoxy)-tert-butyldimethylsilane (3.38 mL, 14.58 mmol) was added dropwise. The reaction mixture was allowed to warm up to RT and stirred for 16 h. The reaction mixture was concentrated under reduced pressure, diluted with water and extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 90/10 as eluent to afford tert-butyl-[3-(4,5-dibromotriazol-2-yl)propoxy]-dimethyl-silane as a colorless liquid.

LCMS method F: [M+H]⁺=398.2-400.1-402.1, t_{R}=3.69 min

### Preparation of Intermediate 433: 3-(4-bromotriazol-2-yl)propoxy-tert-butyl-dimethyl-silane

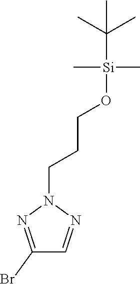 [SMILES: CC(C)(C)[Si](C)(C)OCCCn1ncc(Br)n1]

To a solution of tert-butyl-[3-(4,5-dibromotriazol-2-yl)propoxy]-dimethyl-silane (3.2 g, 8.02 mmol) in anhydrous THF (77 mL) at −20° C. under argon was added dropwise isopropyl magnesium chloride lithium chloride complex (1.3M solution in THF) (6.8 mL, 8.82 mmol). The reaction mixture was allowed warm up to RT and stirred at RT for 16 h. The reaction mixture was cooled to −20° C. and additional isopropylmagnesium chloride lithium chloride complex (1.3M solution in THF) (2.5 mL, 3.20 mmol) was added. The reaction was stirred at RT for 6 h. The reaction mixture was quenched by addition of saturated aqueous ammonium chloride solution and extracted with ethyl acetate. The combined organic layers were washed with water and brine, dried over anhydrous sodium sulfate, filtered and evaporated under reduced pressure. The residue was purified by silica gel column chromatography using cyclohexane/ethyl acetate 100/0 to 90/10 as eluent to afford 3-(4-bromotriazol-2-yl)propoxy-tert-butyl-dimethyl-silane as a yellow liquid.

LCMS method F: [M+H]⁺=320.1-322.1, t_{R}=3.48 min

### Preparation of Intermediate 434: 3-(4-bromotriazol-2-yl)propan-1-ol

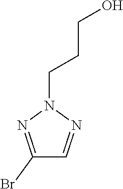

To a solution of 3-(4-bromotriazol-2-yl)propoxy-tert-butyl-dimethyl-silane (1.91 g, 5.96 mmol) in anhydrous THF (18 mL) at RT was added TBAF (1M solution in THF) (6.6 mL, 6.56 mmol). The reaction mixture was stirred at RT for 6 h. The reaction mixture was poured into ice water and stirred for 10 min. The aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 60/40 as eluent to afford 3-(4-bromotriazol-2-yl)propan-1-ol as a colorless oil.

LCMS method F: [M+H]⁺=206.1-208.1, t_{R}=1.52 min

### Preparation of Intermediate 435: 2-[3-(3-benzyloxypropoxy)propyl]-4-bromo-triazole

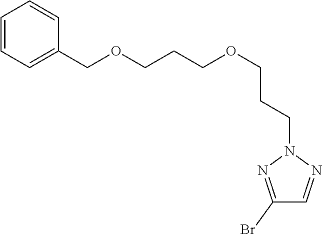 [SMILES: Brc1cnn(CCCOCCCOCc2ccccc2)n1]

To a solution of 3-(4-bromotriazol-2-yl)propan-1-ol (482 mg, 2.340 mmol) in anhydrous DMF (12 mL) at 0° C. was added sodium hydride (60% dispersion in mineral oil) (187 mg, 4.68 mmol). The reaction mixture was stirred at 0° C. for 30 min. A solution of 3-benzyloxypropyl 4-methylbenzenesulfonate (1.335 g, 4.172 mmol) in dry DMF (8 mL) was added dropwise and the reaction mixture was stirred at 65° C. overnight. The reaction mixture was quenched by addition of water and concentrated under reduced pressure. The residue was diluted with a brine and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 90/10 as eluent to afford 2-[3-(3-benzyloxypropoxy)propyl]-4-bromo-triazole as a colorless oil.

LCMS method F: [M+H]⁺=354.1-356.1, t_{R}=2.92 min

### Preparation of Intermediate 436: [3-[2-[3-(3-benzyloxypropoxy)propyl]triazol-4-yl]-1-tetrahydropyran-2-yl-indazol-5-yl]oxy-tert-butyl-dimethyl-silane

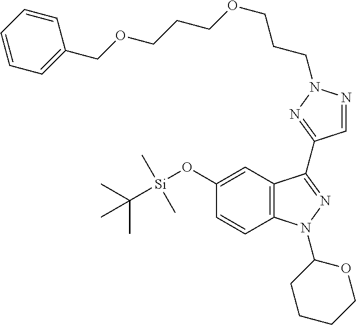 [SMILES: CC(C)(C)[Si](C)(C)Oc1ccc2c(c1)c(-c1cnn(CCCOCCCOCc3ccccc3)n1)nn2C1CCCCO1]

To a degassed suspension of 2-[3-(3-benzyloxypropoxy)propyl]-4-bromo-triazole (803 mg, 2.26 mmol), tert-butyl-dimethyl-[1-tetrahydropyran-2-yl-3-(4,4,5,5-tetramethyl-1,3,2-dioxa borolan-2-yl)indazol-5-yl]oxy-silane (intermediate 61) (1.24 g, 2.72 mmol), potassium phosphate tribasic (1.44 g, 6.80 mmol) and XPhos (108 mg, 0.227 mmol) in dioxane (24 mL) and water (2.4 mL) was added tetrakis(triphenylphosphine)palladium(0) (131 mg, 0.113 mmol). The reaction mixture was stirred at 90° C. under microwave irradiations for 2 h. The reaction mixture was filtered. The filtrate was diluted with brine and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 80/20 as eluent to afford [3-[2-[3-(3-benzyloxypropoxy)propyl]triazol-4-yl]-1-tetrahydropyran-2-yl-indazol-5-yl]oxy-tert-butyl-dimethyl-silane as an orange oil.

LCMS method F: [M+H]⁺=606.4, t_{R}=3.99 min

### Preparation of Intermediate 437: 3-[3-[4-[5-[tert-butyl(dimethyl)silyl]oxy-1-tetra hydropyran-2-yl-indazol-3-yl]triazol-2-yl]propoxy]propan-1-ol

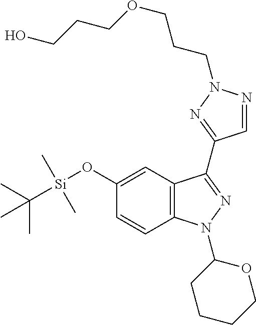 [SMILES: CC(C)(C)[Si](C)(C)Oc1ccc2c(c1)c(-c1cnn(CCCOCCCO)n1)nn2C1CCCCO1]

To a solution of [3-[2-[3-(3-benzyloxypropoxy)propyl]triazol-4-yl]-1-tetrahydropyran-2-yl-indazol-5-yl]oxy-tert-butyl-dimethyl-silane (1.057 g, 1.74 mmol) in ethanol (40 mL) at RT was added palladium 10% on carbon (106 mg). The reaction mixture was stirred under hydrogen atmosphere at RT for 16 h. The reaction mixture was filtered, washed with ethanol and the solvent was removed under reduced pressure. The residue was purified by silica gel column chromatography using cyclohexane/ethyl acetate 100/0 to 60/40 as eluent to afford 3-[3-[4-[5-[tert-butyl(dimethyl)silyl]oxy-1-tetrahydropyran-2-yl-indazol-3-yl]triazol-2-yl]propoxy] propan-1-ol as a colorless oil.

LCMS method F: [M+H]⁺=516.3, t_{R}=3.40 min

### Preparation of Intermediate 438: 3-[3-[4-[5-[tert-butyl(dimethyl)silyl]oxy-1-tetra hydropyran-2-yl-indazol-3-yl]triazol-2-yl]propoxy]propyl methanesulfonate

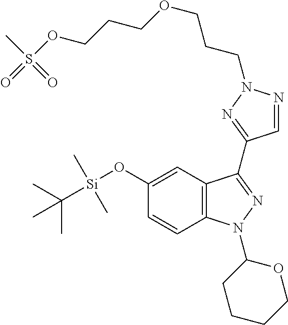 [SMILES: CC(C)(C)[Si](C)(C)Oc1ccc2c(c1)c(-c1cnn(CCCOCCCOS(C)(=O)=O)n1)nn2C1CCCCO1]

To a solution of 3-[3-[4-[5-[tert-butyl(dimethyl)silyl]oxy-1-tetrahydropyran-2-yl-indazol-3-yl]triazol-2-yl]propoxy]propan-1-ol (737 mg, 1.431 mmol) and triethylamine (400 μL, 2.862 mmol) in dry dichloromethane (33 mL) at 0° C. was added methanesulfonyl chloride (166 μL, 2.147 mmol). The reaction mixture was stirred at RT for 16 h. The reaction mixture was quenched with brine and extracted with dichloromethane. The combined organic layers were dried over anhydrous sodium sulfate, filtered and evaporated under reduced pressure to afford 3-[3-[4-[5-[tert-butyl(dimethyl)silyl]oxy-1-tetrahydropyran-2-yl-indazol-3-yl]triazol-2-yl] propoxy]propyl methanesulfonate as a pale orange oil which was used in the next step without further purification.

LCMS method F: [M+H]⁺=594.4, t_{R}=3.56 min

### Preparation of Intermediate 439: 18-(oxan-2-yl)-9,13-dioxa-4,5,18,19,22-pentaaza tetracyclo[12.5.2.1^{2,5}.0^{17,20}]docosa-1(19),2(22),3,14(21),15,17(20)-hexaene

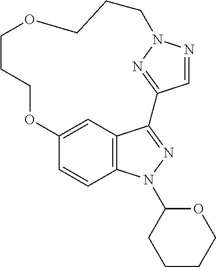 [SMILES: c1cc2c3cc1OCCCOCCCn1ncc(n1)-c3nn2C1CCCCO1]

To a suspension of cesium carbonate (1.865 g, 5.724 mmol) in dry DMF (500 mL) at 85° C. was added dropwise a solution of 3-[3-[4-[5-[tert-butyl(dimethyl)silyl]oxy-1-tetrahydropyran-2-yl-indazol-3-yl]triazol-2-yl]propoxy]propyl methanesulfonate (871 mg, 1.431 mmol) in dry DMF (500 mL). The reaction mixture was heated to 85° C. for 6 h. The reaction mixture was concentrated under reduced pressure then diluted with brine and ethyl acetate. The aqueous layer was extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 70/30 as eluent to afford 18-(oxan-2-yl)-9,13-dioxa-4,5,18,19,22-pentaazatetracyclo[12.5.2.1^{2,5}.0^{17,20}]docosa-1(19),2(22),3,14(21),15,17(20)-hexaene as a colorless oil.

LCMS method F: [M+H]⁺=384.4, t_{R}=2.70 min

### Preparation of Example 74: 9,13-dioxa-4,5,18,19,22-pentaazatetracyclo[12.5.2.1^{2,5}.0^{17,20}]docosa-1(19),2(22),3,14(21),15,17(20)-hexaene

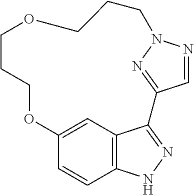 [SMILES: c1cc2[nH]nc3c2cc1OCCCOCCCn1ncc-3n1]

To a solution of 18-(oxan-2-yl)-9,13-dioxa-4,5,18,19,22-pentaazatetracyclo [12.5.2.1^{2,5}.0^{17,20}] docosa-1(19),2(22),3,14(21),15,17(20)-hexaene (422 mg, 1.102 mmol) in methanol (54 mL) and water (7.3 mL) at RT was added p-toluenesulfonic acid monohydrate (1.048 g, 5.51 mmol). The reaction mixture was stirred at 65° C. for 16 h. The reaction mixture was concentrated under reduced pressure and the residue was neutralized by addition of a saturated aqueous NaHCO₃ solution. The residue was diluted with ethyl acetate. The phases were separated and the aqueous phase was extracted with ethyl acetate. The combined organic layers were washed with brine, dried over sodium sulfate, filtered and evaporated under reduced pressure. The residue was triturated with diisopropyl ether, filtered, washed with diisopropyl ether and dried to afford 9,13-dioxa-4,5,18,19,22-pentaazatetracyclo[12.5.2.1^{2,5}.0^{17,20}]docosa-1(19),2(22),3,14(21),15, 17(20)-hexaene as a powder.

LCMS method F: [M+H]⁺=300, t_{R}=2.02 min

LCMS method G: [M+H]⁺=300, t_{R}=2.01 min

1H NMR (400 MHz, d6-DMSO) 13.05 (1H, s), 8.11 (1H, s), 8.08 (1H, d, J=2.3 Hz), 7.47-7.44 (1H, d, J=8.9 Hz), 7.01-6.98 (1H, dd, J=2.5, 8.9 Hz), 4.58-4.53 (2H, m), 4.38-4.32 (2H, t, J=7.6 Hz), 3.83-3.78 (2H, t, J=7.1 Hz), 3.63-3.59 (2H, m), 2.38-2.30 (2H, m), 2.08-2.01 (2H, m) ppm.

### Example 75: (13R)-13-methyl-7,10,14-trioxa-23-thia-4,19,20-triazatetracyclo[13.5.2.1^{2,5}.0^{18,21}]tricosa-1(20),2,4,15(22),16,18(21)-hexaene

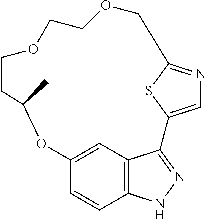 [SMILES: C[C@@H]1CCOCCOCC2=NC=C(S2)c2n[nH]c3ccc(cc23)O1]

Example 75 is prepared according to the synthesis route described in general Scheme D, following the same synthesis procedures as for Example 66.

### Preparation of Intermediate 440: (13R)-13-methyl-19-(oxan-2-yl)-7,10,14-trioxa-23-thia-4,19,20-triazatetracyclo[13.5.2.1^{2,5}.0^{18,21}]tricosa-1(20),2,4,15(22),16,18(21)-hexaene

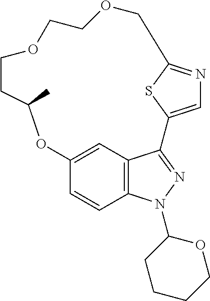 [SMILES: C[C@@H]1CCOCCOCC2=NC=C(S2)c2nn(C3CCCCO3)c3ccc(cc23)O1]

To a suspension of cesium carbonate (742 mg, 2.276 mmol) in anhydrous DMF (230 mL) at 80° C. was added dropwise a solution of [(1S)-3-[2-[[5-[5-[tert-butyl(dimethyl)silyl]oxy-1-tetrahydropyran-2-yl-indazol-3-yl]thiazol-2-yl]methoxy]ethoxy]-1-methyl-propyl]methane sulfonate (390 mg, 0.569 mmol) in anhydrous DMF (230 mL). The reaction mixture was stirred at 80° C. for 16 h. The reaction mixture was concentrated under reduced pressure, diluted with brine and extracted with ethyl acetate. The combined organic layers were dried over anhydrous sodium sulfate, filtered and evaporated under reduced pressure. The residue was purified by silica gel column chromatography using dichloromethane/ethyl acetate 100/0 to 70/30 as eluent to afford (13R)-13-methyl-19-(oxan-2-yl)-7,10,14-trioxa-23-thia-4,19,20-triazatetracyclo [13.5.2.1^{2,5}.0^{18,21}]tricosa-1(20),2,4,15(22),16,18(21)-hexaene as a colorless oil.

LCMS method F: [M+H]⁺=430.2, t_{R}=2.93 min

### Preparation of Example 75: (13R)-13-methyl-7,10,14-trioxa-23-thia-4,19,20-triaza tetracyclo[13.5.2.1^{2,5}.0^{18,21}]tricosa-1(20),2,4,15(22),16,18(21)-hexaene

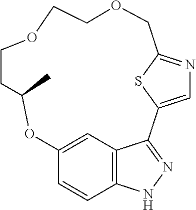 [SMILES: C[C@@H]1CCOCCOCC2=NC=C(S2)c2n[nH]c3ccc(cc23)O1]

To a solution of (13R)-13-methyl-19-(oxan-2-yl)-7,10,14-trioxa-23-thia-4,19,20-triazatetra cyclo[13.5.2.1^{2,5}.0^{18,21}]tricosa-1(20),2,4,15(22),16,18(21)-hexaene (57 mg, 0.133 mmol) in methanol (7.9 mL) and water (1.1 mL) at RT was added p-toluenesulfonic acid monohydrate (126 mg, 0.665 mmol). The reaction mixture was stirred at 65° C. for 16 h. The reaction mixture was concentrated under reduced pressure and the residue was neutralized by addition of a saturated aqueous NaHCO₃ solution. ethyl acetate was added, the phases were separated and the aqueous layer was extracted ethyl acetate. The combined organic layers were washed with brine, dried over anhydrous sodium sulfate, filtered and evaporated under reduced pressure. The residue was purified by preparative TLC eluting with cyclohexane/(ethyl acetate/ethanol) (3-1) 50/50 as eluent. The resulting oil was triturated with diisopropyl ether to afford (13R)-13-methyl-7,10,14-trioxa-23-thia-4,19,20-triazatetracyclo[13.5.2.1^{2,5}.0^{18,21}]tricosa-1(20),2,4, 15(22),16,18(21)-hexaene as a powder.

LCMS method F: [M+H]⁺=346, t_{R}=2.25 min

LCMS method G: [M+H]⁺=346, t_{R}=2.24 min

1H NMR (400 MHz, MeOD) 8.00 (1H, s), 7.65 (1H, d, J=2.3 Hz), 7.48-7.45 (1H, d, J=9.0 Hz), 7.07-7.04 (1H, dd, J=2.4, 9.0 Hz), 5.08-5.04 (1H, d, J=15.6 Hz), 4.73-4.69 (1H, d, J=15.8 Hz), 4.62-4.55 (1H, m), 3.89-3.69 (5H, m), 3.60-3.52 (1H, m), 2.69-2.60 (1H, m), 1.62-1.53 (1H, m), 1.45 (3H, d, J=5.9 Hz) ppm.

### Example 76: (13R)-4,13-dimethyl-8,11,14-trioxa-4,19,20,23-tetraazatetracyclo[13.5.2.1^{2,5}.0^{18,21}]tricosa-1(20),2,5(23),15(22),16,18(21)-hexaene

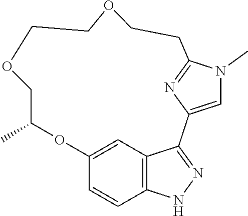

Example 76 is prepared according to the synthesis route described in general Scheme D.

### Preparation of Intermediate 441: tert-butyl-[2-(1H-imidazol-2-yl)ethoxy]-dimethyl-silane

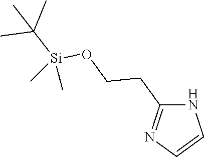

To a solution of 2-(1H-imidazol-2-yl)ethanol (1.2 g, 10.7 mmol) in DMF (11.5 mL) at RT was added imidazole (1.09 g, 16.05 mmol) followed by the addition of tert-butyldimethylsilyl chloride (1.61 g, 10.7 mmol). The reaction mixture was stirred at RT overnight. The mixture was poured in water and extracted with ethyl acetate. The combined organic layers were washed with 10% aqueous lithium chloride solution, water, brine then dried over anhydrous sodium sulfate, filtered and evaporated under reduced pressure to afford tert-butyl-[2-(1H-imidazol-2-yl)ethoxy]-dimethyl-silane as a yellow oil which was used in the next step without any further purification.

LCMS method F: [M+H]⁺=227.3, t_{R}=1.64 min

### Preparation of Intermediate 442: tert-butyl-[2-(4,5-dibromo-1H-imidazol-2-yl) ethoxy]-dimethyl-silane

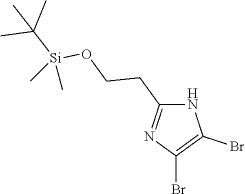 [SMILES: CC(C)(C)[Si](C)(C)OCCc1nc(Br)c(Br)[nH]1]

To a suspension of tert-butyl-[2-(1H-imidazol-2-yl)ethoxy]-dimethyl-silane (2.379 g, 10.52 mmol) in THF (350 mL) at RT was added N-bromosuccinimide (1.955 g, 11.05 mmol). The mixture was stirred at RT for 30 min. The reaction mixture was diluted with ethyl acetate, washed with water and brine, dried over anhydrous magnesium sulfate, filtered and the solvent was removed under reduced pressure. The residue was purified by silica gel column chromatography using cyclohexane/ethyl acetate 100/0 to 95/5 as eluent to afford tert-butyl-[2-(4,5-dibromo-1H-imidazol-2-yl)ethoxy]-dimethyl-silane as a colorless oil.

LCMS method F: [M+H]⁺=383.1-385.1-387.0, t_{R}=3.00 min

### Preparation of Intermediate 443: tert-butyl-[2-(4,5-dibromo-1-methyl-imidazol-2-yl)ethoxy]-dimethyl-silane

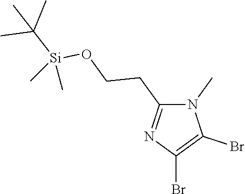 [SMILES: Cn1c(CCO[Si](C)(C)C(C)(C)C)nc(Br)c1Br]

To a solution of tert-butyl-[2-(4,5-dibromo-1H-imidazol-2-yl)ethoxy]-dimethyl-silane (928 mg, 2.42 mmol) in DMF (3.9 mL) at RT was added potassium carbonate (734 mg, 5.32 mmol) followed by iodomethane (180 μL, 2.9 mmol). The reaction mixture was stirred at RT overnight. The reaction mixture was diluted with dichloromethane, washed with water and brine, dried over anhydrous sodium sulfate, filtered and the solvent was removed under reduced pressure to afford tert-butyl-[2-(4,5-dibromo-1-methyl-imidazol-2-yl)ethoxy]-dimethyl-silane as a cream solid which was used in the next step without any further purification.

LCMS method F: [M+H]⁺=397.0-399.1-401.1, t_{R}=3.25 min

### Preparation of Intermediate 444: 2-(4-bromo-1-methyl-imidazol-2-yl)ethoxy-tert-butyl-dimethyl-silane

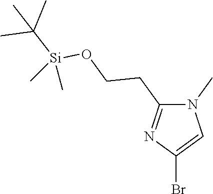 [SMILES: Cn1cc(Br)nc1CCO[Si](C)(C)C(C)(C)C]

To a degassed solution of tert-butyl-[2-(4,5-dibromo-1-methyl-imidazol-2-yl)ethoxy]-dimethyl-silane (975 mg, 2.42 mmol) in dry THF (38 mL) at −78° C. was added n-BuLi (2.5 M solution in hexane) (1.32 mL, 3.3 mmol) and the reaction mixture was stirred at −78° C. for 30 min. Additional n-BuLi (2.5 M solution in hexane) (100 μL, 0.24 mmol) was added and the reaction mixture was stirred at −78° C. for 3 h. Additional n-BuLi (2.5 M solution in hexane) (100 μL, 0.24 mmol) was added and the mixture was stirred at −78° C. for 45 min. The reaction mixture was poured in a saturated aqueous ammonium chloride solution at 0° C. The aqueous layer was extracted with ethyl acetate. The combined organic layers were washed with brine, dried over anhydrous sodium sulfate, filtered and evaporated under reduced pressure to afford 2-(4-bromo-1-methyl-imidazol-2-yl)ethoxy-tert-butyl-dimethyl-silane as a brown oil which was used in the next step without further purification.

LCMS method F: [M+H]⁺=319.2-321.2, t_{R}=2.60 min

### Preparation of Intermediate 445: 2-(4-bromo-1-methyl-imidazol-2-yl)ethanol

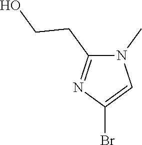

To a solution of 2-(4-bromo-1-methyl-imidazol-2-yl)ethoxy-tert-butyl-dimethyl-silane (617 mg, 1.93 mmol) in THF (8 mL) at RT was added dropwise TBAF (1 M solution in THF) (2.13 mL, 2.13 mmol). The reaction mixture was stirred at RT overnight. The reaction mixture was poured into ice water. The aqueous layer was neutralized to pH 7 and extracted with ethyl acetate. The combined organic layers were dried over anhydrous sodium sulfate, filtered and evaporated under reduced pressure. The residue was purified by silica gel column chromatography using dichloromethane/methanol 100/0 to 95/5 as eluent to afford 2-(4-bromo-1-methyl-imidazol-2-yl)ethanol as a cream solid.

¹H NMR (400 MHz, CDCl₃): 6.81 (1H, s), 4.06 (2H, m), 3.5 (1H, m), 2.84 (2H, t, J=5.5 Hz), 1.57 (3H, s) ppm.

### Preparation of Intermediate 446: 2-[2-[2-[(2S)-2-benzyloxypropoxy]ethoxy]ethyl]-4-bromo-1-methyl-imidazole

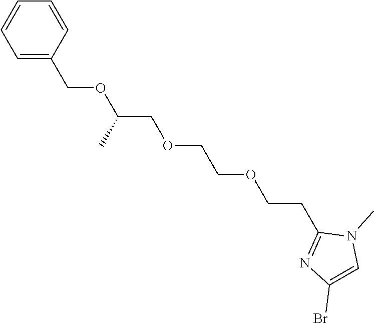 [SMILES: C[C@@H](COCCOCCc1nc(Br)cn1C)OCc1ccccc1]

To a solution of 2-(4-bromo-1-methyl-imidazol-2-yl)ethanol (207 mg, 1.01 mmol) in dry DMF (3 mL) was added sodium hydride (60% dispersion in mineral in oil) (48 mg, 1.2 mmol). The reaction mixture was stirred at RT for 20 min and a solution of 2-[(2S)-2-benzyloxypropoxy]ethylmethanesulfonate (obtained using the same process as for intermediate 401) (346 mg, 1.2 mmol) in dry DMF (1.6 mL) was added. The reaction mixture was stirred at RT overnight then quenched with water and extracted with ethyl acetate. The combined organic layers were washed with brine, dried over anhydrous sodium sulfate, filtered and evaporated under reduced pressure to afford 2-[2-[2-[(2S)-2-benzyloxypropoxy]ethoxy]ethyl]-4-bromo-1-methyl-imidazole as a pale yellow oil which was used in the next step without further purification.

LCMS method F: [M+H]⁺=397.2-399.2, t_{R}=2.18 min

### Preparation of Intermediate 447: [3-[2-[2-[2-[(2S)-2-benzyloxypropoxy]ethoxy] ethyl]-1-methyl-imidazol-4-yl]-1-tetrahydropyran-2-yl-indazol-5-yl]oxy-tert-butyl-dimethyl-silane

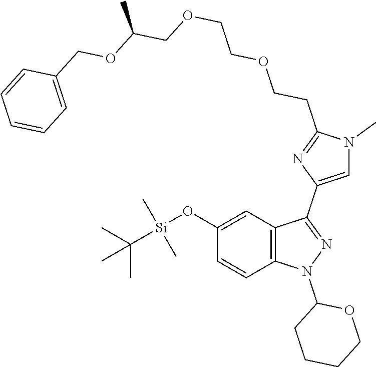 [SMILES: C[C@@H](COCCOCCc1nc(-c2nn(C3CCCCO3)c3ccc(O[Si](C)(C)C(C)(C)C)cc23)cn1C)OCc1ccccc1]

To a degassed suspension of 2-[2-[2-[(2S)-2-benzyloxypropoxy]ethoxy]ethyl]-4-bromo-1-methyl-imidazole (401 mg, 1.01 mmol), tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (647 mg, 1.42 mmol), tripotassium phosphate (642 mg, 3.03 mmol) and XPhos (48 mg, 0.101 mmol) in dioxane (8.5 mL) and water (0.4 mL) was added tetrakis(triphenylphosphine)palladium(0) (58 mg, 0.051 mmol). The reaction mixture was stirred at 140° C. for 1.5 h. The reaction mixture was diluted with water and extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 0/100 as eluent to afford [3-[2-[2-[2-[(2S)-2-benzyloxypropoxy]ethoxy]ethyl]-1-methyl-imidazol-4-yl]-1-tetrahydropyran-2-yl-indazol-5-yl]oxy-tert-butyl-dimethyl-silane as a pale brown viscous oil.

LCMS method F: [M+H]⁺=649.6, t_{R}=2.80 min

### Preparation of Intermediate 448: (2S)-1-[2-[2-[4-[5-[tert-butyl(dimethyl)silyl]oxy-1-tetrahydropyran-2-yl-indazol-3-yl]-1-methyl-imidazol-2-yl]ethoxy]ethoxy]propan-2-ol

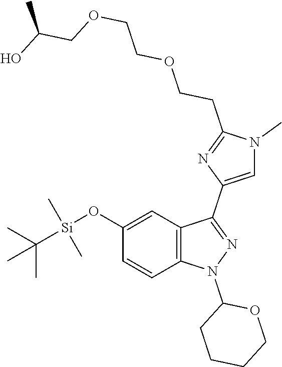 [SMILES: C[C@H](O)COCCOCCc1nc(-c2nn(C3CCCCO3)c3ccc(O[Si](C)(C)C(C)(C)C)cc23)cn1C]

To a solution of [3-[2-[2-[2-[(2S)-2-benzyloxypropoxy]ethoxy]ethyl]-1-methyl-imidazol-4-yl]-1-tetrahydropyran-2-yl-indazol-5-yl]oxy-tert-butyl-dimethyl-silane (183 mg, 0.28 mmol) in ethanol (6 mL) at RT was added palladium hydroxide on carbon (18 mg). The reaction mixture was stirred under hydrogen atmosphere at RT for 20 h. The reaction mixture was stirred at 60° C. for 48 h. The reaction mixture was filtered and the filtrate was concentrated and dried under reduced pressure to afford (2S)-1-[2-[2-[4-[5-[tert-butyl(dimethyl)silyl]oxy-1-tetrahydro pyran-2-yl-indazol-3-yl]-1-methyl-imidazol-2-yl]ethoxy]ethoxy]propan-2-ol as a brown oil.

LCMS method F: [M+H]⁺=559.4, t_{R}=2.45 min

### Preparation of Intermediate 449: [(1S)-2-[2-[2-[4-[5-[tert-butyl(dimethyl)silyl]oxy-1-tetrahydropyran-2-yl-indazol-3-yl]-1-methyl-imidazol-2-yl]ethoxy]ethoxy]-1-methyl-ethyl] methanesulfonate

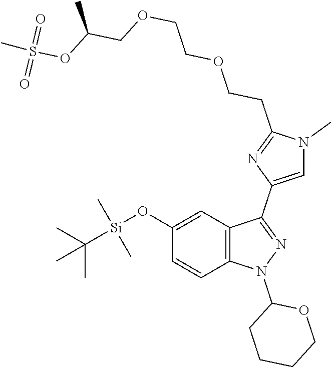

To a solution of (2S)-1-[2-[2-[4-[5-[tert-butyl(dimethyl)silyl]oxy-1-tetrahydropyran-2-yl-indazol-3-yl]-1-methyl-imidazol-2-yl]ethoxy]ethoxy]propan-2-ol (163 mg, 0.28 mmol) in dichloromethane (3.5 mL) at 0° C. was added triethylamine (79 μL, 0.56 mol) and methanesulfonyl chloride (26 μL, 0.34 mmol). The reaction mixture was stirred at RT overnight. Additional triethylamine (79 μL, 0.56 mol) and methanesulfonyl chloride (26 μL, 0.34 mmol) were added and the reaction mixture was stirred at RT for 3 h. The reaction mixture was diluted with water and extracted with dichloromethane. The combined organic layers were washed with a saturated aqueous ammonium chloride solution and brine, dried over anhydrous sodium sulfate, filtered and evaporated under reduced pressure to afford [(1S)-2-[2-[2-[4-[5-[tert-butyl(dimethyl)silyl]oxy-1-tetrahydropyran-2-yl-indazol-3-yl]-1-methyl-imidazol-2-yl] ethoxy]ethoxy]-1-methyl-ethyl] methanesulfonate as a pale brown oil which was used in the next step without further purification.

LCMS method F: [M+H]⁺=637.4, t_{R}=2.58 min

### Preparation of Intermediate 450: (13R)-4,13-dimethyl-19-(oxan-2-yl)-8,11,14-trioxa-4,19,20,23-tetraazatetracyclo[13.5.2.1^{2,5}.0^{18,21}]tricosa-1(20),2,5(23),15(22),16,18(21)-hexaene

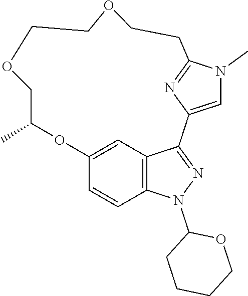

To a suspension of cesium carbonate (225 mg, 0.69 mmol) in anhydrous DMF (20 mL) at 60° C. was added dropwise [(1S)-2-[2-[2-[4-[5-[tert-butyl(dimethyl)silyl]oxy-1-tetrahydropyran-2-yl-indazol-3-yl]-1-methyl-imidazol-2-yl]ethoxy]ethoxy]-1-methyl-ethyl] methanesulfonate (147 mg, 0.23 mmol) in DMF (20 mL). The reaction mixture was stirred at 60° C. for 2 h. The reaction mixture was filtered and the solvent was removed under reduced pressure. The residue was purified by silica gel column chromatography using cyclohexane/(ethyl acetate/ethanol (3/1)) 100/0 to 80/20 as eluent to afford (13R)-4,13-dimethyl-19-(oxan-2-yl)-8,11,14-trioxa-4,19,20, 23-tetraazatetracyclo[13.5.2.1^{2,5}.0^{18,21}]tricosa-1(20),2,5(23),15(22),16,18(21)-hexaene as a white/cream solid.

LCMS method F: [M+H]⁺=427.4, t_{R}=1.64 min

### Preparation of Example 76: (13R)-4,13-dimethyl-8,11,14-trioxa-4,19,20,23-tetraazatetracyclo[13.5.2.1^{2,5}.0^{18,21}]tricosa-1(20),2,5(23),15(22),16,18(21)-hexaene

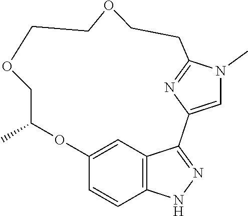

To a solution of (13R)-4,13-dimethyl-19-(oxan-2-yl)-8,11,14-trioxa-4,19,20,23-tetraazatetra cyclo[13.5.2.1^{2,5}.0^{18,21}]tricosa-1(20),2,5(23),15(22),16,18(21)-hexaene (59 mg, 0.14 mmol) in methanol (2.4 mL) and water (0.4 mL) was added p-toluenesulfonic acid monohydrate (132 mg, 0.69 mmol) and the reaction mixture was stirred at 65° C. overnight. The reaction mixture was diluted with dichloromethane and saturated aqueous NaHCO₃ solution. The aqueous layer was extracted with dichloromethane. The combined organic layers were washed with brine, dried over anhydrous sodium sulfate, filtered and evaporated under reduced pressure. The residue was triturated with diisopropyl ether, filtered, washed with diisopropyl ether and dried to afford (13R)-4,13-dimethyl-8,11,14-trioxa-4,19,20,23-tetraazatetracyclo[13.5.2.1^{2,5}.0^{18,21}] tricosa-1(20),2,5(23),15(22),16,18(21)-hexaene as a solid.

LCMS method F: [M+H]⁺=343.3, t_{R}=1.31 min

LCMS method G: [M+H]⁺=343.3, t_{R}=1.74 min

1H NMR (400 MHz, CDCl₃) 9.65 (1H, s), 8.42 (1H, m), 7.29 (2H, m), 7.07 (1H, dd, J=2.5, 8.9 Hz), 4.47 (1H, m), 4.20 (2H, m), 4.02 (1H, m), 3.89 (1H, dd, J=5.4, 9.6 Hz), 3.83 (1H, m), 3.77 (1H, m), 3.71 (1H, m), 3.64 (3H, s), 3.48 (1H, dd, J=4.6, 9.7 Hz), 3.09 (1H, m), 2.95 (1H, m), 1.41 (3H, d, J=6.6 Hz) ppm.

### Example 77: (7R)-7-methyl-8,11,14-trioxa-4,5,19,20-tetraazatetracyclo[13.5.2.1^{2,5}.0^{18,21}] tricosa-1(20),2(23),3,15(22),16,18(21)-hexaene

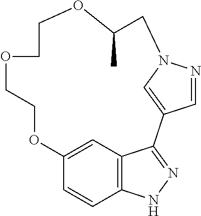 [SMILES: C[C@@H]1Cn2cc(cn2)-c2n[nH]c3ccc(cc23)OCCOCCO1]

Example 77 is prepared according to the synthesis route described in general Scheme D.

### Preparation of Intermediate 451: (2R)-4-trityloxybutan-2-ol

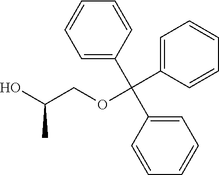 [SMILES: C[C@@H](O)COC(c1ccccc1)(c1ccccc1)c1ccccc1]

To a solution of (2R)-propane-1,2-diol (962 μL, 13.14 mmol) in dichloromethane (30 mL) at 0° C. was added triethylamine (2.381 mL, 17.09 mmol) followed by dropwise addition of trityl chloride (3.664 g, 13.14 mmol) in dichloromethane (10 mL). The reaction mixture was stirred at RT overnight. Water was added and the aqueous layer was extracted with dichloromethane. The organic layer was washed with brine, dried over anhydrous sodium sulfate, filtered and evaporated under reduced pressure to afford (2R)-4-trityloxybutan-2-ol as a colorless oil.

LCMS method F: [M+Na]⁺=341.2, t_{R}=3.01 min

### Preparation of Intermediate 452: [[(2R)-2-[2-(2-benzyloxyethoxy)ethoxy]propoxy]-diphenyl-methyl]benzene

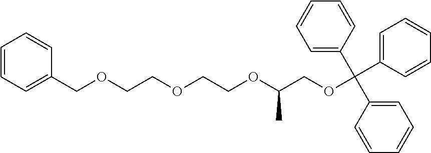 [SMILES: C[C@H](COC(c1ccccc1)(c1ccccc1)c1ccccc1)OCCOCCOCc1ccccc1]

To a solution of (2R)-4-trityloxybutan-2-ol (1.2 g, 3.77 mmol) in DMF (15 mL) at 0° C. was added sodium hydride (60% dispersion in mineral oil) (226 mg, 5.65 mmol) and 2-[2-(benzyloxy)ethoxy]ethyl methanesulfonate (3.102 g, 11.31 mmol) in DMF (3 mL). The reaction mixture was stirred at RT for 16 h. The mixture was diluted with ethyl acetate, washed with water and brine. The organic layer was dried over anhydrous sodium sulfate, filtered and evaporated under reduced pressure to afford [[(2R)-2-[2-(2-benzyloxyethoxy)ethoxy]propoxy]-diphenyl-methyl]benzene as a light yellow oil which was used in the next step without further purification.

LCMS method F: [M+Na]⁺=519.3, t_{R}=3.63 min

### Preparation of Intermediate 453: (2R)-2-{2-[2-(benzyloxy)ethoxy]ethoxy}propan-1-ol

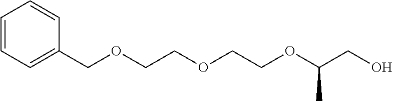 [SMILES: C[C@H](CO)OCCOCCOCc1ccccc1]

To a solution of [[(2R)-2-[2-(2-benzyloxyethoxy)ethoxy]propoxy]-diphenyl-methyl]benzene (1.872 g, 3.77 mmol) in a mixture of dichloromethane/methanol (4 mL) was added p-toluenesulfonic acid monohydrate (72 mg, 0.38 mmol). The reaction mixture was stirred at RT overnight. The solvent was removed under reduced pressure. The residue was purified by silica gel column chromatography using cyclohexane/ethyl acetate 100/0 to 90/10 as eluent to afford (2R)-2-{2-[2-(benzyloxy)ethoxy]ethoxy}propan-1-ol as a colorless oil.

LCMS method F: [M+H]⁺=255.3, t_{R}=1.89 min

### Preparation of Intermediate 454: [(2R)-2-[2-(2-benzyloxyethoxy)ethoxy]propyl] 4-methylbenzenesulfonate

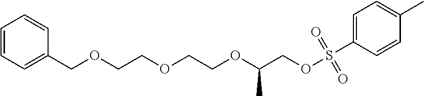 [SMILES: Cc1ccc(S(=O)(=O)OC[C@@H](C)OCCOCCOCc2ccccc2)cc1]

To a suspension (2R)-2-{2-[2-(benzyloxy)ethoxy]ethoxy}propan-1-ol (300 mg, 1.18 mmol) in pyridine (6 mL) at 0° C. was added p-toluenesulfonyl chloride (270 mg, 1.42 mmol). The reaction mixture was stirred at RT overnight. Ethyl acetate was added and the organic layer washed with water and brine. The organic layer was dried over anhydrous sodium sulfate, filtered and evaporated under reduced pressure to afford [(2R)-2-[2-(2-benzyloxyethoxy) ethoxy]propyl]4-methylbenzenesulfonate as a light yellow oil which was used in the next step without further purification.

LCMS method F: [M+H]⁺=409.4, t_{R}=2.88 min

### Preparation of Intermediate 455: 1-[(2R)-2-{2-[2-(benzyloxy)ethoxy]ethoxy}propyl]-4-bromo-1H-pyrazole

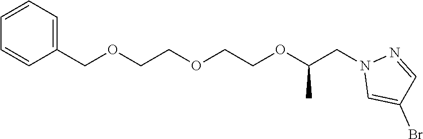 [SMILES: C[C@H](Cn1cc(Br)cn1)OCCOCCOCc1ccccc1]

To a solution of 4-bromo-1H-pyrazole (193 mg, 1.31 mmol) in acetonitrile (8 mL) were added cesium carbonate (556 mg, 1.71 mmol) and [(2R)-2-[2-(2-benzyloxyethoxy)ethoxy]propyl] 4-methyl benzenesulfonate (480 mg, 1.44 mmol). The reaction mixture was stirred at 85° C. for 4 h. Water and ethyl acetate were added, the phases were separated and the aqueous layer was extracted with ethyl acetate. The combined organic layers were dried over anhydrous sodium sulfate, filtered and evaporated under reduced pressure. The residue was purified by silica gel column chromatography using cyclohexane/ethyl acetate 80/20 as eluent to afford 1-[(2R)-2-{2-[2-(benzyloxy)ethoxy]ethoxy}propyl]-4-bromo-1H-pyrazole as a colorless oil.

LCMS method F: [M+H]⁺=383.3-385.3, t_{R}=2.67 min

### Preparation of Intermediate 456: [3-[1-[(2R)-2-[2-(2-benzyloxyethoxy)ethoxy] propyl]pyrazol-4-yl]-1-tetrahydropyran-2-yl-indazol-5-yl]oxy-tert-butyl-dimethyl-silane

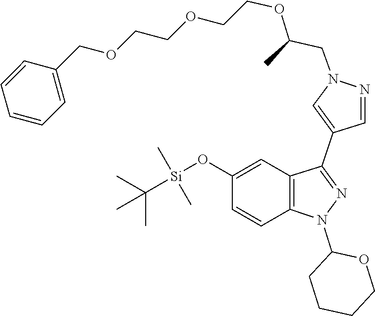 [SMILES: C[C@H](Cn1cc(-c2nn(C3CCCCO3)c3ccc(O[Si](C)(C)C(C)(C)C)cc23)cn1)OCCOCCOCc1ccccc1]

To a suspension of 1-[(2R)-2-{2-[2-(benzyloxy)ethoxy]ethoxy}propyl]-4-bromo-1H-pyrazole (187 mg, 0.49 mmol) in dioxane (2.5 mL) and water (0.3 mL) was added tert-butyl-dimethyl-[1-tetrahydropyran-2-yl-3-(4,4,5,5-tetramethyl-1,3,2-dioxaborolan-2-yl)indazol-5-yl]oxy-silane (291 mg, 0.63 mmol), and potassium phosphate tribasic (311 mg, 1.46 mmol). The reaction mixture was purged with argon for 15 min then tetrakis(triphenyl phosphine)palladium(0) (28 mg, 0.02 mmol) and Xphos (23 mg, 0.05 mmol) were added. The reaction mixture was stirred at 90° C. under microwave irradiations for 1.5 h. The reaction mixture was poured into water and extracted with ethyl acetate. The combined organic layers were washed with brine, dried over anhydrous sodium sulfate and evaporated under reduced pressure. The residue was purified by silica gel column chromatography using cyclohexane/ethyl acetate 80/20 as eluent to afford [3-[1-[(2R)-2-[2-(2-benzyloxyethoxy) ethoxy]propyl]pyrazol-4-yl]-1-tetrahydropyran-2-yl-indazol-5-yl]oxy-tert-butyl-dimethyl-silane as a light brown oil.

LCMS method F: [M+H]⁺=635.5, t_{R}=3.74 min

### Preparation of Intermediate 457: 2-[2-[(1R)-2-[4-[5-[tert-butyl(dimethyl)silyl]oxy-1-tetrahydropyran-2-yl-indazol-3-yl]pyrazol-1-yl]-1-methyl-ethoxy]ethoxy]ethanol

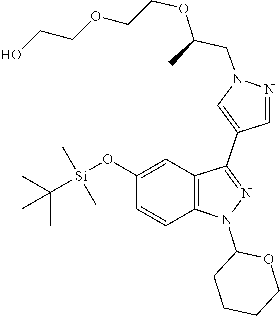 [SMILES: C[C@H](Cn1cc(-c2nn(C3CCCCO3)c3ccc(O[Si](C)(C)C(C)(C)C)cc23)cn1)OCCOCCO]

To a solution of [3-[1-[(2R)-2-[2-(2-benzyloxyethoxy)ethoxy]propyl]pyrazol-4-yl]-1-tetra hydropyran-2-yl-indazol-5-yl]oxy-tert-butyl-dimethyl-silane (116 mg, 0.18 mmol) in ethanol (5 mL) under argon was added palladium on carbon 10% Wt (12 mg, 0.11 mmol). The reaction mixture was stirred under hydrogen atmosphere at RT for 96 h. The reaction mixture was filtered and the solvent was removed under reduced pressure to afford 2-[2-[(1R)-2-[4-[5-[tert-butyl(dimethyl)silyl]oxy-1-tetrahydropyran-2-yl-indazol-3-yl]pyrazol-1-yl]-1-methyl-ethoxy] ethoxy]ethanol as a light yellow oil which was used in the next step without further purification.

LCMS method F: [M+H]⁺=545.4, t_{R}=3.19 min

### Preparation of Intermediate 458: 2-[2-[(1R)-2-[4-[5-[tert-butyl(dimethyl)silyl]oxy-1-tetrahydropyran-2-yl-indazol-3-yl]pyrazol-1-yl]-1-methyl-ethoxy]ethoxy]ethyl methanesulfonate

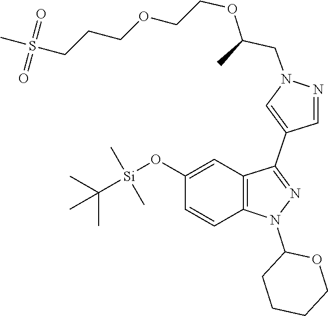 [SMILES: C[C@H](Cn1cc(-c2nn(C3CCCCO3)c3ccc(O[Si](C)(C)C(C)(C)C)cc23)cn1)OCCOCCCS(C)(=O)=O]

To a suspension 2-[2-[(1R)-2-[4-[5-[tert-butyl(dimethyl)silyl]oxy-1-tetrahydropyran-2-yl-indazol-3-yl]pyrazol-1-yl]-1-methyl-ethoxy]ethoxy]ethanol (100 mg, 0.18 mmol) in dichloromethane (2 mL) at 0° C. was added triethylamine (38 μL, 0.28 mmol) and methanesulfonyl chloride (16 μL, 0.20 mmol). The reaction mixture was stirred at RT for 1 h. The reaction mixture was diluted with water, the phases were separated and the aqueous layer was extracted with dichloromethane. The combined organic layers were washed with a saturated aqueous ammonium chloride solution, water, brine, dried over anhydrous sodium sulfate, filtered and evaporated under reduced pressure to afford 2-[2-[(1R)-2-[4-[5-[tert-butyl(dimethyl)silyl]oxy-1-tetrahydropyran-2-yl-indazol-3-yl]pyrazol-1-yl]-1-methyl-ethoxy] ethoxy]ethyl methanesulfonate as a light yellow oil which was used in the next step without further purification.

LCMS method F: [M+H]⁺=623.5, t_{R}=3.36 min

### Preparation of Intermediate 459: (7R)-7-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene

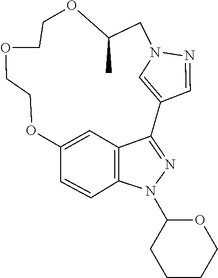 [SMILES: C[C@@H]1Cn2cc(cn2)-c2nn(C3CCCCO3)c3ccc(cc23)OCCOCCO1]

To a suspension of cesium carbonate (157 mg, 0.48 mmol) in anhydrous DMF (4 mL) at 80° C. was added dropwise 2-[2-[(1R)-2-[4-[5-[tert-butyl(dimethyl)silyl]oxy-1-tetrahydropyran-2-yl-indazol-3-yl]pyrazol-1-yl]-1-methyl-ethoxy]ethoxy]ethylmethanesulfonate (100 mg, 0.16 mmol) in DMF (2 mL). The reaction mixture was stirred at 80° C. for 30 min. The reaction mixture was filtered and concentrated under reduced pressure. The residue was purified by silica gel column chromatography using cyclohexane/ethyl acetate 50/50 as eluent to afford (7R)-7-methyl-19-(oxan-2-yl)-8,11,14-trioxa-4,5,19,20-tetraazatetracyclo[13.5.2.1^{2,5}.0^{18,21}] tricosa-1(20),2(23),3,15(22),16,18(21)-hexaene as a yellow oil.

LCMS method F: [M+H]⁺=413.4, t_{R}=2.48 min

### Preparation of Example 77: (7R)-7-methyl-8,11,14-trioxa-4,5,19,20-tetraazatetra cyclo[13.5.2.1^{2,5}.0^{18,21}]tricosa-1(20),2(23),3,15(22),16,18(21)-hexaene

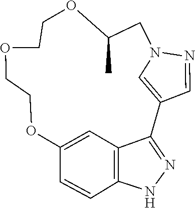 [SMILES: C[C@@H]1Cn2cc(cn2)-c2n[nH]c3ccc(cc23)OCCOCCO1]

To a solution of (7R)-7-methyl-19-(oxan-2-yl)-8,11,14-trioxa-4,5,19,20-tetraazatetracyclo [13.5.2.1^{2,5}.0^{18,21}]tricosa-1(20),2(23),3,15(22),16,18(21)-hexaene (27 mg, 0.07 mmol) in methanol (1.3 mL) and water (0.2 mL) was added p-toluenesulfonic acid monohydrate (62 mg, 0.33 mmol) and the reaction mixture was stirred at 80° C. for 16 h. The reaction mixture was dissolved in ethyl acetate and a saturated aqueous NaHCO₃ solution was added. The aqueous layer was extracted with ethyl acetate. The combined organic layers were washed with brine, dried over anhydrous sodium sulfate, filtered and evaporated under reduced pressure. the resulting product was recrystallized from acetonitrile, filtered and dried under reduced pressure to afford (7R)-7-methyl-8,11,14-trioxa-4,5,19,20-tetraazatetracyclo[13.5.2.1^{2,5}.0^{18,21}]tricosa-1(20),2(23),3,15(22),16,18(21)-hexaene as a solid.

LCMS method F: [M+H]⁺=329.3, t_{R}=1.91 min

LCMS method G: [M+H]⁺=329.3, t_{R}=1.91 min

1H NMR (400 MHz, CDCl₃) 8.57 (1H, s), 8.23-8.21 (1H, m), 8.05-8.03 (1H, m), 7.35 (1H, s), 7.12-7.08 (1H, m), 4.51-4.38 (3H, m), 4.26-4.19 (2H, m), 3.91-3.83 (2H, m), 3.77-3.56 (4H, m), 1.32-1.26 (4H, m) ppm.

### Example 78: (13,13-difluoro-8,11,14-trioxa-4,5,19,20-tetraazatetracyclo[13.5.2.1^{2,5}.0^{18,21}] tricosa-1(20),2(23),3,15(22),16,18(21)-hexaene

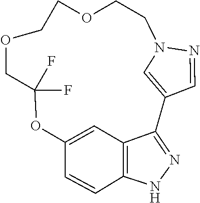 [SMILES: FC1(F)COCCOCCn2cc(cn2)-c2n[nH]c3ccc(cc23)O1]

Example 78 is prepared according to the synthesis route described in general Scheme F.

### Preparation of Intermediate 460: 2,2-difluoro-2-[1-tetrahydropyran-2-yl-3-[1-(2-trimethylsilylethoxymethyl) pyrazol-4-yl]indazol-5-yl]oxy-acetic acid

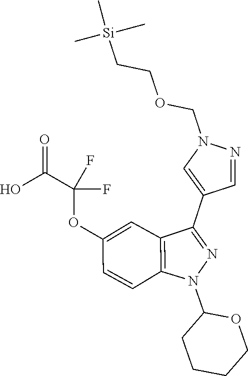 [SMILES: C[Si](C)(C)CCOCn1cc(-c2nn(C3CCCCO3)c3ccc(OC(F)(F)C(=O)O)cc23)cn1]

To a degassed solution of 1-tetrahydropyran-2-yl-3-[1-(2-trimethylsilylethoxymethyl)pyrazol-4-yl]indazol-5-ol (intermediate 229) (1.06 g, 2.56 mmol) in THF (18 mL) at 0° C. was added sodium hydride (60% dispersion in mineral oil) (188 mg, 2.82 mmol). After 15 min, tert-butyl 2-bromo-2,2-difluoro-acetate (709 mg, 3.07 mmol) was added and the reaction mixture was stirred at 55° C. overnight. The reaction mixture was quenched with water, extracted with ethyl acetate. The combined organic layers were washed with a saturated aqueous NaHCO₃ solution and brine then dried over anhydrous sodium sulfate, filtered and evaporated under reduced pressure to afford 2,2-difluoro-2-[1-tetrahydropyran-2-yl-3-[1-(2-trimethylsilylethoxymethyl) pyrazol-4-yl]indazol-5-yl]oxy-acetic acid as a brown paste which was used in the next step without further purification.

LCMS method F: [M+H]⁺=509.3, t_{R}=3.45 min

### Preparation of Intermediate 461: 2,2-difluoro-2-[1-tetrahydropyran-2-yl-3-[1-(2-trimethylsilylethoxymethyl)pyrazol-4-yl]indazol-5-yl]oxy-ethanol

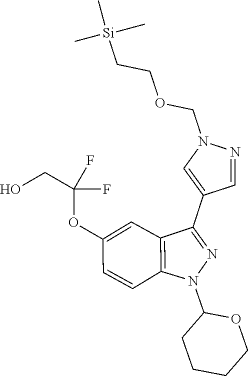 [SMILES: C[Si](C)(C)CCOCn1cc(-c2nn(C3CCCCO3)c3ccc(OC(F)(F)CO)cc23)cn1]

To a degassed solution of 2,2-difluoro-2-[1-tetrahydropyran-2-yl-3-[1-(2-trimethylsilylethoxy methyl)pyrazol-4-yl]indazol-5-yl]oxy-acetic acid (1.12 g, 2.2 mmol) in THF (15 mL) at 0° C. was added borane dimethylsulfide (1 M solution in Me-THF) (4.4 mL, 4.4 mmol). The reaction mixture was stirred at RT for 24 h. Additional borane dimethylsulfide (1 M solution in Me-THF) (4.4 mL, 4.4 mmol) was added at 0° C. and the reaction was stirred at RT overnight. The reaction mixture was quenched with methanol at 0° C. then water and ethyl acetate were added. The aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 60/40 as eluent to afford 2,2-difluoro-2-[1-tetrahydropyran-2-yl-3-[1-(2-trimethylsilylethoxymethyl)pyrazol-4-yl]indazol-5-yl]oxy-ethanol as a colorless viscous oil.

LCMS method J: [M+H]⁺=495.4, t_{R}=4.54 min

### Preparation of Intermediate 462: 2-[[4-[5-[2-[2-(2-benzyloxyethoxy)ethoxy]-1,1-difluoro-ethoxy]-1-tetrahydropyran-2-yl-indazol-3-yl]pyrazol-1-yl]methoxy]ethyl-trimethyl-silane

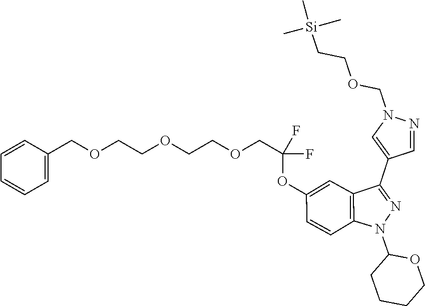 [SMILES: C[Si](C)(C)CCOCn1cc(-c2nn(C3CCCCO3)c3ccc(OC(F)(F)COCCOCCOCc4ccccc4)cc23)cn1]

To a degassed solution of 2,2-difluoro-2-[1-tetrahydropyran-2-yl-3-[1-(2-trimethylsilylethoxy methyl)pyrazol-4-yl]indazol-5-yl]oxy-ethanol (434 mg, 0.88 mmol) and 2-(2-benzyloxy ethoxy)ethyl methanesulfonate (intermediate 145) (321 mg, 1.1 mmol) in dry DMF (9 mL) at 0° C. was added sodium hydride (60% dispersion in mineral oil) (70 mg, 1.76 mmol). The reaction was stirred at RT overnight. The reaction mixture was quenched with water and extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 60/40 as eluent to afford 2-[[4-[5-[2-[2-(2-benzyloxyethoxy)ethoxy]-1,1-difluoro-ethoxy]-1-tetra hydropyran-2-yl-indazol-3-yl]pyrazol-1-yl]methoxy]ethyl-trimethyl-silane as a colorless oil.

LCMS method F: [M+H]⁺=673.6, t_{R}=3.55 min

### Preparation of Intermediate 463: 2-[2-[2,2-difluoro-2-[1-tetrahydropyran-2-yl-3-[1-(2-trimethylsilylethoxymethyl)pyrazol-4-yl]indazol-5-yl]oxy-ethoxy]ethoxy]ethanol

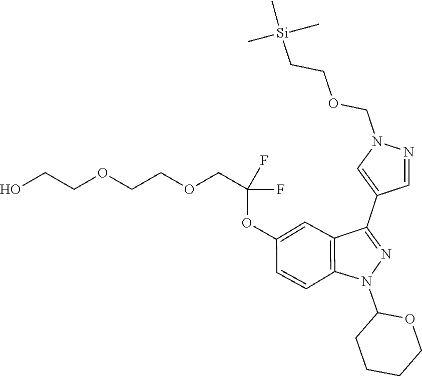 [SMILES: C[Si](C)(C)CCOCn1cc(-c2nn(C3CCCCO3)c3ccc(OC(F)(F)COCCOCCO)cc23)cn1]

To a solution of 2-[[4-[5-[2-[2-(2-benzyloxyethoxy)ethoxy]-1,1-difluoro-ethoxy]-1-tetrahydro pyran-2-yl-indazol-3-yl]pyrazol-1-yl]methoxy]ethyl-trimethyl-silane (500 mg, 0.74 mmol) in ethanol (11 mL) at RT was added palladium on carbon (50 mg). The reaction mixture was stirred under hydrogen atmosphere at RT for 5 h. The reaction mixture filtered and the solvent was removed under reduced pressure to afford 2-[2-[2,2-difluoro-2-[1-tetrahydropyran-2-yl-3-[1-(2-trimethylsilylethoxymethyl)pyrazol-4-yl]indazol-5-yl]oxy-ethoxy]ethoxy]ethanol as a colorless oil which was used in the next step without further purification.

LCMS method F: [M+H]⁺=583.4, t_{R}=3.01 min

### Preparation of Intermediate 464: 2-[2-[2,2-difluoro-2-[3-(1H-pyrazol-4-yl)-1-tetrahydropyran-2-yl-indazol-5-yl]oxy-ethoxy]ethoxy]ethanol

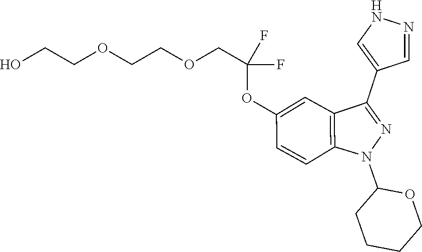 [SMILES: OCCOCCOCC(F)(F)Oc1ccc2c(c1)c(-c1cn[nH]c1)nn2C1CCCCO1]

To a solution of 2-[2-[2,2-difluoro-2-[1-tetrahydropyran-2-yl-3-[1-(2-trimethylsilylethoxy methyl)pyrazol-4-yl]indazol-5-yl]oxy-ethoxy]ethoxy]ethanol (347 mg, 0.6 mmol) in THF (7 mL) at RT was added TBAF (1M solution in THF) (1.5 mL, 1.5 mmol). The reaction mixture was stirred at 60° C. for 48 h. The reaction mixture was quenched with a saturated aqueous ammonium chloride solution and extracted with ethyl acetate. The combined organic layers were washed with brine, dried over anhydrous sodium sulfate, filtered and evaporated under reduced pressure to afford 2-[2-[2,2-difluoro-2-[3-(1H-pyrazol-4-yl)-1-tetrahydropyran-2-yl-indazol-5-yl]oxy-ethoxy]ethoxy]ethanol as a brown oil which was used in the next step without further purification.

LCMS method F: [M+H]⁺=453.4, t_{R}=2.08 min

### Preparation of Intermediate 465: 2-[2-[2,2-difluoro-2-[3-(1H-pyrazol-4-yl)-1-tetra hydropyran-2-yl-indazol-5-yl]oxy-ethoxy]ethoxy]ethyl4-methylbenzenesulfonate

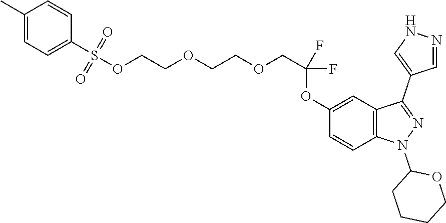 [SMILES: Cc1ccc(S(=O)(=O)OCCOCCOCC(F)(F)Oc2ccc3c(c2)c(-c2cn[nH]c2)nn3C2CCCCO2)cc1]

To a solution of 2-[2-[2,2-difluoro-2-[3-(1H-pyrazol-4-yl)-1-tetrahydropyran-2-yl-indazol-5-yl]oxy-ethoxy]ethoxy]ethanol (299 mg, 0.55 mmol), triethylamine (100 □L, 0.83 mmol) and DMAP (3 mg, 0.028 mmol) in dichloromethane (5.5 mL) at 0° C. was added portionwise p-toluenesulfonyl chloride (94 mg, 0.5 mmol). The reaction mixture was stirred at RT overnight. Additional triethylamine (15 μL, 0.11 mmol) and p-toluenesulfonyl chloride (11 mg, 0.055 mmol) were added. The reaction mixture was stirred at RT for 4 h. The reaction mixture was diluted with a saturated aqueous ammonium chloride solution and extracted with dichloromethane. The combined organic layers were washed with brine, dried over anhydrous sodium sulfate, filtered and evaporated under reduced pressure to afford 2-[2-[2,2-difluoro-2-[3-(1H-pyrazol-4-yl)-1-tetrahydropyran-2-yl-indazol-5-yl]oxy-ethoxy]ethoxy]ethyl4-methyl benzene sulfonate as a brown oil which was used in the next step without further purification.

LCMS method F: [M+H]⁺=607.4, t_{R}=2.88 min

### Preparation of Intermediate 466: 13,13-difluoro-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene

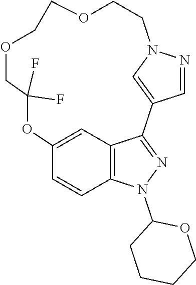 [SMILES: FC1(F)COCCOCCn2cc(cn2)-c2nn(C3CCCCO3)c3ccc(cc23)O1]

To a degassed solution of sodium hydride (60% dispersion in mineral oil) (220 mg, 5.5 mmol) in DMF (90 mL) at 60° C. was added dropwise 2-[2-[2,2-difluoro-2-[3-(1H-pyrazol-4-yl)-1-tetrahydropyran-2-yl-indazol-5-yl]oxy-ethoxy]ethoxy]ethyl-4-methylbenzenesulfonate (404 mg, 0.55 mmol) in DMF (90 mL). The reaction mixture was stirred at 60° C. overnight. The reaction mixture was quenched with methanol and the solvent was removed under reduced pressure. The residue was purified by silica gel column chromatography using dichloromethane/methanol 100/0 to 97/3 as eluent to afford 13,13-difluoro-19-(oxan-2-yl)-8,11,14-trioxa-4,5,19,20-tetraazatetracyclo[13.5.2.1^{2,5}.0^{18,21}]tricosa-1(20),2(23),3,15(22),16, 18(21)-hexaene as a pale white solid.

LCMS method F: [M+H]⁺=435.4, t_{R}=2.61 min

### Preparation of Example 78: 13,13-difluoro-8,11,14-trioxa-4,5,19,20-tetraazatetra cyclo[13.5.2.1^{2,5}.0^{18,21}]tricosa-1(20),2(23),3, 15(22),16,18(21)-hexaene

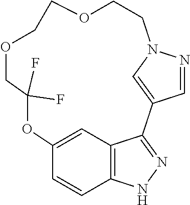 [SMILES: FC1(F)COCCOCCn2cc(cn2)-c2n[nH]c3ccc(cc23)O1]

To a solution of 13,13-difluoro-19-(oxan-2-yl)-8,11,14-trioxa-4,5,19,20-tetraazatetracyclo [13.5.2.1^{2,5}.0^{18,21}]tricosa-1(20),2(23),3,15(22),16,18(21)-hexaene (26 mg, 0.059 mmol) in methanol (1 mL) and water (0.17 mL) was added p-toluenesulfonic acid monohydrate (57 mg, 0.3 mmol). The reaction mixture was stirred at 65° C. for 23 h. The reaction mixture was diluted with dichloromethane and a saturated aqueous NaHCO₃ solution. The aqueous layer was extracted with dichloromethane. The combined organic layers were washed brine, dried over anhydrous sodium sulfate, filtered and evaporated under reduced pressure. The residue was triturated with diethyl ether, filtered and washed with diethyl ether and dried to afford 13,13-difluoro-8,11,14-trioxa-4,5,19,20-tetraazatetracyclo[13.5.2.1^{2,5}.0^{18,21}]tricosa-1(20),2(23),3, 15(22),16,18(21)-hexaene as a powder.

LCMS method F: [M+H]⁺=351.3, t_{R}=2.06 min

LCMS method G: [M+H]⁺=351.3, t_{R}=2.05 min

1H NMR (400 MHz, CDCl₃) 8.66 (1H, s), 8.26 (1H, d, J=1.9 Hz), 8.13 (1H, s), 7.57 (1H, d, J=9 Hz), 7.37 (1H, dd, J=2.2, 9 Hz), 4.54 (2H, m), 3.87 (8H, m) ppm.

### Example 79: (13S)-4,13-dimethyl-7,10,14-trioxa-4,19,20,23-tetraazatetracyclo[13.5.2.1^{2,5}.0^{18,21}]tricosa-1(20),2,5(23),15(22),16,18(21)-hexaene

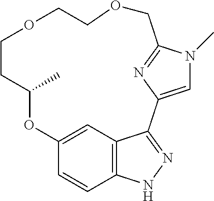 [SMILES: C[C@H]1CCOCCOCc2nc(cn2C)-c2n[nH]c3ccc(cc23)O1]

Example 79 is prepared according to the synthesis route described in general Scheme D.

### Preparation of Intermediate 467: (4-bromo-1-methyl-imidazol-2-yl)methanol

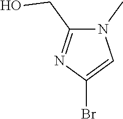

To a suspension of methyl 4-bromo-1-methyl-imidazole-2-carboxylate (730 mg, 3.35 mmol) in methanol (30 mL) at 0° C. was added portionwise sodium borohydride (279 mg, 7.37 mmol). The reaction mixture was stirred at 0° C. for 2 h. Additional sodium borohydride (279 mg, 7.37 mmol) was added at 0° C. and the reaction mixture was stirred at RT for 3 h. Water was added and methanol was removed under reduced pressure. The aqueous layer was extracted with ethyl acetate and the organic layer was washed with brine, dried over anhydrous sodium sulfate, filtered and evaporated under reduced pressure to afford (4-bromo-1-methyl-imidazol-2-yl)methanol as a white solid which was used in the next step without further purification.

LCMS method H: [M+H]⁺=191.1-193.1, t_{R}=0.86 min

### Preparation of Intermediate 468: 2-[2-[(3R)-3-benzyloxybutoxy]ethoxymethyl]-4-bromo-1-methyl-imidazole

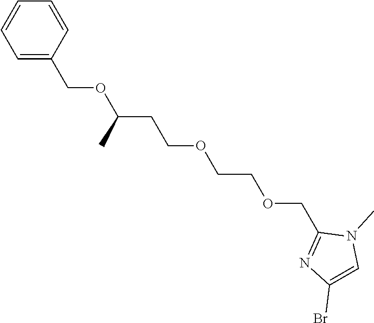 [SMILES: C[C@H](CCOCCOCc1nc(Br)cn1C)OCc1ccccc1]

To a solution of (4-bromo-1-methyl-imidazol-2-yl)methanol (286 mg, 1.95 mmol) in dry acetonitrile (15 mL) at RT was added cesium carbonate (3.18 g, 9.75 mmol) and 2-[(3R)-3-benzyloxybutoxy]ethyl-4-methylbenzene sulfonate (intermediate 346) (720 mg, 2.15 mmol). The reaction was stirred to 70° C. for 120 h in a sealed tube. The reaction mixture was concentrated under reduced pressure. The residue was dissolved with water and ethyl acetate. The phases were separated and the aqueous layer was extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 50/50 as eluent to afford 2-[2-[(3R)-3-benzyloxybutoxy] ethoxymethyl]-4-bromo-1-methyl-imidazole as a colorless oil.

LCMS method F: [M+H]⁺=397.1-399.1, t_{R}=2.54 min

### Preparation of Intermediate 469: [3-[2-[2-[(3R)-3-benzyloxybutoxy]ethoxymethyl]-1-methyl-imidazol-4-yl]-1-tetrahydropyran-2-yl-indazol-5-yl]oxy-tert-butyl-dimethyl-silane

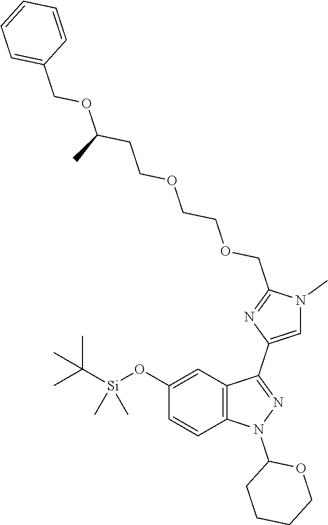 [SMILES: C[C@H](CCOCCOCc1nc(-c2nn(C3CCCCO3)c3ccc(O[Si](C)(C)C(C)(C)C)cc23)cn1C)OCc1ccccc1]

To a degassed solution of tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (774 mg, 1.69 mmol), 2-[2-[(3R)-3-benzyloxybutoxy]ethoxymethyl]-4-bromo-1-methyl-imidazole (536 mg, 1.35 mmol) and potassium phosphate tribasic (859 mg, 4.05 mmol) in dioxane (20 mL) and water (1 mL) was added tetrakis(triphenylphosphine)palladium(0) (78 mg, 0.0675 mmol). The reaction mixture was stirred at 100° C. for 2 h. The solvent was removed under reduced pressure and water and ethyl acetate were added. The aqueous layer was extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 50/50 as eluent to afford [3-[2-[2-[(3R)-3-benzyloxybutoxy]ethoxymethyl]-1-methyl-imidazol-4-yl]-1-tetrahydropyran-2-yl-indazol-5-yl]oxy-tert-butyl-dimethyl-silane as a yellow oil.

LCMS method F: [M+H]⁺=649.5, t_{R}=3.04 min

### Preparation of Intermediate 470: (2R)-4-[2-[[4-[5-[tert-butyl(dimethyl)silyl]oxy-1-tetrahydropyran-2-yl-indazol-3-yl]-1-methyl-imidazol-2-yl]methoxy]ethoxy]butan-2-ol

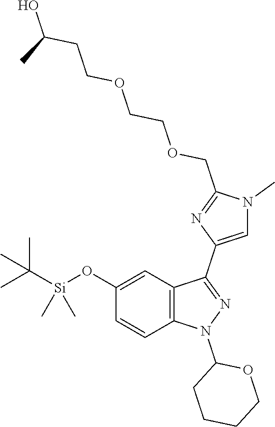 [SMILES: C[C@@H](O)CCOCCOCc1nc(-c2nn(C3CCCCO3)c3ccc(O[Si](C)(C)C(C)(C)C)cc23)cn1C]

To a solution of [3-[2-[2-[(3R)-3-benzyloxybutoxy]ethoxymethyl]-1-methyl-imidazol-4-yl]-1-tetrahydropyran-2-yl-indazol-5-yl]oxy-tert-butyl-dimethyl-silane (408 mg, 0.629 mmol) in ethanol (20 mL) at RT was added palladium hydroxide on carbon (40 mg). The reaction mixture was stirred under hydrogen atmosphere at 65° C. for 16 h. The reaction mixture was filtered and the filtrate was evaporated under reduced pressure to afford (2R)-4-[2-[[4-[5-[tert-butyl(dimethyl)silyl]oxy-1-tetrahydropyran-2-yl-indazol-3-yl]-1-methyl-imidazol-2-yl] methoxy]ethoxy]butan-2-ol as a yellow oil which was used in the next step without further purification.

LCMS method F: [M+H]⁺=559.4, t_{R}=2.54 min

### Preparation of Intermediate 471: [(JR)-3-[2-[[4-[5-[tert-butyl(dimethyl)silyl]oxy-1-tetrahydropyran-2-yl-indazol-3-yl]-1]-methyl-imidazol-2-yl]methoxy]ethoxyl-1-methyl-propyl] methanesulfonate

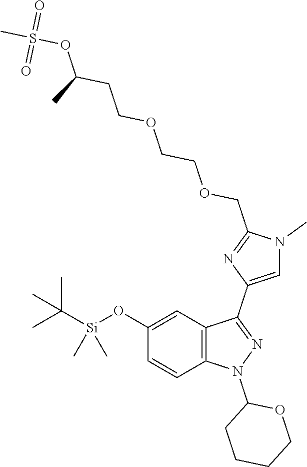

To a solution of (2R)-4-[2-[[4-[5-[tert-butyl(dimethyl)silyl]oxy-1-tetrahydropyran-2-yl-indazol-3-yl]-1-methyl-imidazol-2-yl]methoxy]ethoxy]butan-2-ol (330 mg, 0.59 mmol) in dichloromethane (20 mL) at 0° C. was added triethylamine (164 μL, 1.18 mmol) and methanesulfonyl chloride (55 μL, 0.709 mmol). The reaction mixture was stirred at RT overnight. Additional triethylamine (164 μL, 1.18 mol) and methanesulfonyl chloride (55 μL, 0.709 mmol) were added and the reaction mixture was stirred at RT for 3 h. The reaction mixture was diluted with water and extracted with dichloromethane. The combined organic layers were washed with a saturated aqueous ammonium chloride solution and brine, dried over anhydrous sodium sulfate, filtered and evaporated under reduced pressure to provide the [(1R)-3-[2-[[4-[5-[tert-butyl(dimethyl)silyl]oxy-1-tetrahydropyran-2-yl-indazol-3-yl]-1-methyl-imidazol-2-yl]methoxy] ethoxy]-1-methyl-propyl] methanesulfonate as a colorless oil which was used in the next step without further purification.

LCMS method F: [M+H]⁺=637.5, t_{R}=2.65 min

### Preparation of Intermediate 472: (13S)-4,13-dimethyl-19-(oxan-2-yl)-7,10,14-trioxa-4,19,20,23-tetraazatetracyclo[13.5.2.1^{2,5}.0^{18,21}]tricosa-1(20),2,5(23),15(22),16,18(21)-hexaene

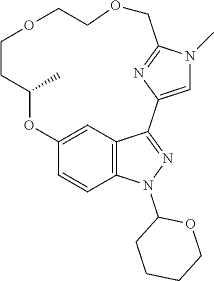 [SMILES: C[C@H]1CCOCCOCc2nc(cn2C)-c2nn(C3CCCCO3)c3ccc(cc23)O1]

To a mixture of sodium hydride (60% dispersion in mineral oil) (66 mg, 1.65 mmol) in DMF (140 mL) was added a solution of [(1R)-3-[2-[[4-[5-[tert-butyl(dimethyl)silyl]oxy-1-tetrahydro pyran-2-yl-indazol-3-yl]-1-methyl-imidazol-2-yl]methoxy]ethoxy]-1-methyl-propyl]methane sulfonate (350 mg, 0.55 mmol) in DMF (140 mL). The reaction mixture was stirred at RT overnight. The solvent was removed under reduced pressure and the residue was dissolved in ethyl acetate and washed with water. The phases were separated and the organic layer was dried over anhydrous sodium sulfate, filtered and evaporated under reduced pressure to afford (13S)-4,13-dimethyl-19-(oxan-2-yl)-7,10,14-trioxa-4,19,20,23-tetraazatetracyclo[13.5.2.1^{2,5}.0^{18,21}] tricosa-1(20),2,5(23),15(22),16,18(21)-hexaene as a yellow oil which was used in the next step without further purification.

LCMS method F: [M+H]⁺=427.3, t_{R}=1.83 min

### Preparation of Example 79: (13S)-4,13-dimethyl-7,10,14-trioxa-4,19,20,23-tetraaza tetracyclo[13.5.2.1^{2,5}.0^{18,21}]tricosa-1(20),2,5(23),15(22),16,18(21)-hexaene

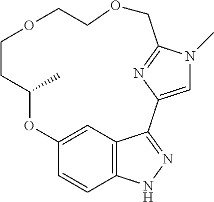 [SMILES: C[C@H]1CCOCCOCc2nc(cn2C)-c2n[nH]c3ccc(cc23)O1]

To a solution of (13S)-4,13-dimethyl-19-(oxan-2-yl)-7,10,14-trioxa-4,19,20,23-tetraazatetra cyclo[13.5.2.1^{2,5}.0^{18,21}]tricosa-1(20),2,5(23),15(22),16,18(21)-hexaene (178 mg, 0.42 mmol) in methanol (5 mL) and water (0.5 mL) was added p-toluenesulfonic acid monohydrate (397 mg, 2.09 mmol). The reaction mixture was stirred at 65° C. for 2 h. The reaction mixture was concentrated under reduced pressure and the residue was neutralized by addition of a saturated aqueous NaHCO₃ solution. The residue was diluted with ethyl acetate. The phases were separated and the aqueous phase was extracted with ethyl acetate. The combined organic layers were washed with a saturated aqueous NaHCO₃ solution, water and brine, dried over anhydrous sodium sulfate, filtered and evaporated under reduced pressure. The residue was purified by silica gel column chromatography using cyclohexane/ethyl acetate 100/0 to 50/50 as eluent.

The resulting product was triturated with diethyl ether, filtered and dried to afford (13S)-4,13-dimethyl-7,10,14-trioxa-4,19,20,23-tetraazatetracyclo[13.5.2.1^{2,5}.0^{18,21}]tricosa-1(20),2,5(23),15(22),16,18(21)-hexaene as a cream solid.

LCMS method F: [M+H]⁺=343.3, t_{R}=1.41 min

LCMS method G: [M+H]⁺=343.3, t_{R}=1.89 min

1H NMR (400 MHz, CDCl₃) 8.17-8.12 (1H, m), 7.28 (2H, s), 7.04-7.00 (1H, m), 4.86 (1H, d, J=15.8 Hz), 4.76 (2H, d, J=14.6 Hz), 4.20-4.15 (1H, m), 3.88-3.78 (3H, m), 3.68-3.66 (4H, m), 3.50 (2H, s), 2.49-2.44 (1H, m), 1.681-1.501 (1H, m), 1.44-1.40 (3H, m) ppm.

### Example 80: (13S)-13-methyl-8,11,14-trioxa-5,19,20,23-tetraazatetracyclo [13.5.2.1^{2,5}.0^{18,21}]tricosa-1(20),2(23),3,15(22),16,18(21)-hexaene

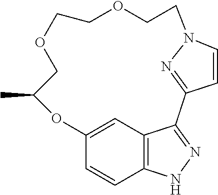 [SMILES: C[C@H]1COCCOCCn2ccc(n2)-c2n[nH]c3ccc(cc23)O1]

Example 80 is prepared according to the synthesis route described in general Scheme D.

### Preparation of Intermediate 473: 2-(3-bromopyrazol-1-yl)ethanol

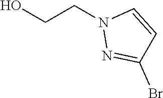

To a solution of ethyl 2-(3-bromopyrazol-1-yl)acetate (500 mg, 2.15 mmol) in methanol (8 mL) was added sodium borohydride (243 mg, 6.44 mmol). The reaction mixture was stirred at RT for 30 min. Water and ethyl acetate were added and the aqueous layer was extracted with ethyl acetate. The combined organic layers were dried over anhydrous sodium sulfate, filtered and evaporated under reduced pressure. The residue was purified on silica gel column chromatography using cyclohexane/ethyl acetate 100/0 to 35/65 as eluent to afford 2-(3-bromopyrazol-1-yl)ethanol as a colorless oil.

LCMS method F: [M+H]⁺=191.0-193.0, t_{R}=1.27 min

### Preparation of Intermediate 474: 1-[2-[2-[(2R)-2-benzyloxypropoxy]ethoxy]ethyl]-3-bromo-pyrazole

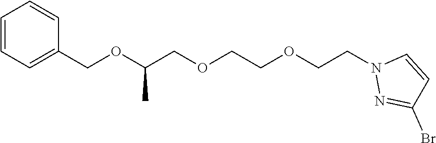 [SMILES: C[C@H](COCCOCCn1ccc(Br)n1)OCc1ccccc1]

To a solution of 2-(3-bromopyrazol-1-yl)ethanol (200 mg, 1.05 mmol) in dry DMF (3.2 mL) was added sodium hydride (60% dispersion in mineral oil) (55 mg, 1.37 mmol). The reaction mixture was stirred at RT for 20 min then 2-[(2R)-2-benzyloxypropoxy]ethyl methanesulfonate (intermediate 387) (515 mg, 1.79 mmol) in dry DMF (1.5 mL) was added. The reaction mixture was stirred at RT for 3 h then at 55° C. for 4 h. Additional sodium hydride (60% dispersion in mineral oil) (55 mg, 1.37 mmol) was added and the reaction was stirred at 55° C. overnight. Water and ethyl acetate were added. The aqueous layer was extracted with ethyl acetate and the combined organic layers were dried over anhydrous sodium sulfate, filtered and evaporated under reduced pressure. The residue was purified by silica gel column chromatography using cyclohexane/ethyl acetate 100/0 to 70/30 as eluent to afford 1-[2-[2-[(2R)-2-benzyloxy propoxy]ethoxy]ethyl]-3-bromo-pyrazole as a colorless oil.

LCMS method F: [M+H]⁺=383.3-385.3, t_{R}=2.70 min

### Preparation of Intermediate 475: [3-[1-[2-[2-[(2R)-2-benzyloxypropoxy]ethoxy] ethyl]pyrazol-3-yl]-1-tetrahydropyran-2-yl-indazol-5-yl]oxy-tert-butyl-dimethyl-silane

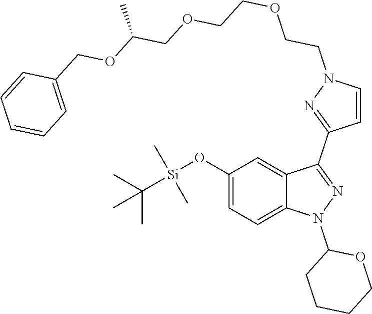 [SMILES: C[C@H](COCCOCCn1ccc(-c2nn(C3CCCCO3)c3ccc(O[Si](C)(C)C(C)(C)C)cc23)n1)OCc1ccccc1]

To a suspension of 1-[2-[2-[(2R)-2-benzyloxypropoxy]ethoxy]ethyl]-3-bromo-pyrazole (204 mg, 0.53 mmol) in dioxane (2.8 mL) and water (0.14 mL) was added tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (291 mg, 0.64 mmol) and potassium phosphate tribasic (337 mg, 1.59 mmol). The mixture was degassed purged with argon for 10 min then tetrakis(triphenylphosphine) palladium(0) (31 mg, 0.03 mmol) and Xphos (25 mg, 0.05 mmol) were added. The reaction mixture was stirred at 90° C. for 3 h. The reaction mixture was cooled to RT and was diluted with water and ethyl acetate. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 70/30 as eluent to afford [3-[1-[2-[2-[(2R)-2-benzyloxypropoxy]ethoxy]ethyl]pyrazol-3-yl]-1-tetrahydropyran-2-yl-indazol-5-yl]oxy-tert-butyl-dimethyl-silane as a pale yellow oil.

LCMS method F: [M+H]⁺=635.7, t_{R}=3.85 min

### Preparation of Intermediate 476: (2R)-1-[2-[2-[3-[5-[tert-butyl(dimethyl)silyl]oxy-1-tetrahydropyran-2-yl-indazol-3-yl]pyrazol-1-yl]ethoxy]ethoxy]propan-2-ol

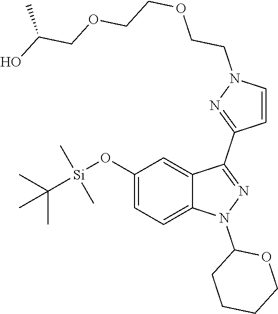 [SMILES: C[C@@H](O)COCCOCCn1ccc(-c2nn(C3CCCCO3)c3ccc(O[Si](C)(C)C(C)(C)C)cc23)n1]

To a suspension of [3-[1-[2-[2-[(2R)-2-benzyloxypropoxy]ethoxy]ethyl]pyrazol-3-yl]-1-tetrahydropyran-2-yl-indazol-5-yl]oxy-tert-butyl-dimethyl-silane (258 mg, 0.41 mmol) in ethanol (3.4 mL) under argon was added palladium on charcoal 10% (25 mg). The reaction mixture was stirred under hydrogen atmosphere at RT for 2 h. The reaction mixture was filtered over a pad of Celite and washed with ethanol and ethyl acetate. The filtrate was evaporated under reduced pressure to afford (2R)-1-[2-[2-[3-[5-[tert-butyl(dimethyl)silyl]oxy-1-tetrahydro pyran-2-yl-indazol-3-yl]pyrazol-1-yl]ethoxy]ethoxy]propan-2-ol as a pale brown oil which was used in the next step without further purification.

LCMS method F: [M+H]⁺=545.5, t_{R}=3.31 min

### Preparation of Intermediate 477: [(1R)-2-[2-[2-[3-[5-[tert-butyl(dimethyl)silyl]oxy-1-tetrahydropyran-2-yl-indazol-3-yl]pyrazol-1-yl]ethoxy]ethoxy]-1-methyl-ethyl] methanesulfonate

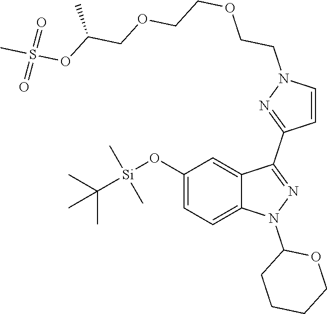 [SMILES: C[C@H](COCCOCCn1ccc(-c2nn(C3CCCCO3)c3ccc(O[Si](C)(C)C(C)(C)C)cc23)n1)OS(C)(=O)=O]

To a solution of (2R)-1-[2-[2-[3-[5-[tert-butyl(dimethyl)silyl]oxy-1-tetrahydropyran-2-yl-indazol-3-yl]pyrazol-1-yl]ethoxy]ethoxy]propan-2-ol (220 mg, 0.40 mmol) in dichloromethane (2.3 mL) at 0° C. was added triethylamine (0.11 mL, 0.80 mmol) and methanesulfonyl chloride (0.04 mL, 0.52 mmol). The mixture was stirred at 0° C. for 10 min and at RT for 5 h. Water was added. The organic layer was washed with a saturated aqueous NaHCO₃ solution and saturated ammonium chloride solution. The organic layer was dried over anhydrous sodium sulfate, filtered and evaporated under reduced pressure. The residue was purified by silica gel column chromatography using cyclohexane/(ethyl acetate/ethanol (3:1)) 100/0 to 50/50 as eluent to afford [(1R)-2-[2-[2-[3-[5-[tert-butyl(dimethyl)silyl]oxy-1-tetrahydropyran-2-yl-indazol-3-yl]pyrazol-1-yl]ethoxy]ethoxy]-1-methyl-ethyl]methanesulfonate as an orange oil.

LCMS method F: [M+H]⁺=623.5, t_{R}=3.47 min

### Preparation of Intermediate 478: (13S)-13-methyl-19-(oxan-2-yl)-8,11,14-trioxa-5,19,20,23-tetraazatetracyclo[13.5.2.1^{2,5}.0^{18,21}]tricosa-1(20),2(23),3,15(22),16,18(21)-hexane

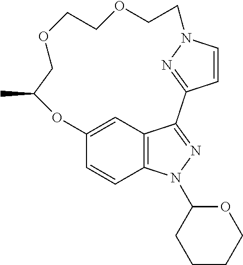 [SMILES: C[C@H]1COCCOCCn2ccc(n2)-c2nn(C3CCCCO3)c3ccc(cc23)O1]

To a solution of cesium carbonate (98 mg, 0.30 mmol) in dry DMF (5.7 mL) at 60° C. was added dropwise a solution of [(1R)-2-[2-[2-[3-[5-[tert-butyl(dimethyl)silyl]oxy-1-tetrahydropyran-2-yl-indazol-3-yl]pyrazol-1-yl]ethoxy]ethoxy]-1-methyl-ethyl] methanesulfonate (60 mg, 0.10 mmol) in dry DMF (2.3 mL). The reaction mixture was stirred at 60° C. for 2 h. The reaction mixture was cooled to RT then filtered through a pad of Celite and washed with ethyl acetate. The solvents were removed under reduced pressure. The residue was purified on silica gel column chromatography using cyclohexane/(ethyl acetate/ethanol (3:1)) 100/0 to 60/40 as eluent to afford (13S)-13-methyl-19-(oxan-2-yl)-8,11,14-trioxa-5,19,20,23-tetraazatetracyclo [13.5.2.1^{2,5}.0^{18,21}]tricosa-1(20),2(23),3,15(22),16,18(21)-hexaene as a colorless oil.

LCMS method F: [M+H]⁺=413.4, t_{R}=2.51 min

### Preparation of Example 80: (13S)-13-methyl-8,11,14-trioxa-5,19,20,23-tetraazatetra cyclo[13.5.2.1^{2,5}.0^{18,21}]tricosa-1(20),2(23),3,15(22),16,18(21)-hexaene

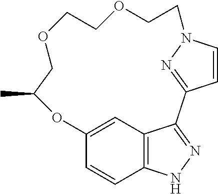 [SMILES: C[C@H]1COCCOCCn2ccc(n2)-c2n[nH]c3ccc(cc23)O1]

To a solution of (13S)-13-methyl-19-(oxan-2-yl)-8,11,14-trioxa-5,19,20,23-tetraazatetracyclo [13.5.2.1^{2,5}.0^{18,21}]tricosa-1(20),2(23),3,15(22),16,18(21)-hexaene (20 mg, 0.048 mmol) in methanol (1 mL) and water (0.15 mL) was added p-toluenesulfonic acid monohydrate (45 mg, 0.24 mmol). The reaction mixture was stirred at 65° C. for 2 h. The reaction mixture was cooled to RT and quenched with a saturated aqueous NaHCO₃ solution until pH basic. ethyl acetate was added, the phases were separated and the aqueous layer was extracted with ethyl acetate. The combined organic layers were dried over anhydrous sodium sulfate, filtered and evaporated under reduced pressure. The residue was purified on silica gel column chromatography using dichloromethane/methanol 100/0 to 97/3 as eluent to afford (13S)-13-methyl-8,11,14-trioxa-5,19,20,23-tetraazatetracyclo[13.5.2.1^{2,5}.0^{18,21}]tricosa-1(20),2(23),3,15(22),16,18(21)-hexaene as a solid.

LCMS method F: [M+H]⁺=329.3, t_{R}=1.87 min

LCMS method G: [M+H]⁺=329.3, t_{R}=1.86 min

1H NMR (400 MHz, CDCl₃) 8.46 (1H, d, J=2.3 Hz), 7.47 (1H, d, J=2.2 Hz), 7.33 (1H, dd, J=0.55 Hz, J=8.9 Hz), 7.08 (1H, dd, J=2.4 Hz, J=8.9 Hz), 6.81 (1H, d, J=2.1 Hz), 4.56-4.34 (4H, m), 3.98-3.60 (6H, m), 3.50 (1H, dd, J=4.2, 9.7 Hz), 1.44 (3H, d, J=6.6 Hz) ppm.

### Example 81: (13S)-13-methyl-8,11,14-trioxa-4,5,19,20,23-pentaazatetracyclo[13.5.2.1^{2,5}.0^{18,21}]tricosa-1(20),2(23),3,15(22),16,18(21)-hexaene

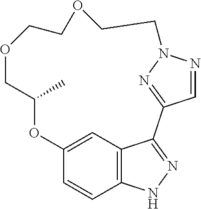 [SMILES: C[C@H]1COCCOCCn2ncc(n2)-c2n[nH]c3ccc(cc23)O1]

Example 81 is prepared according to the synthesis route described in general Scheme D, following the same synthesis procedures as for Example 71.

### Preparation of Intermediate 479: (13S)-13-methyl-19-(oxan-2-yl)-8,11,14-trioxa-4,5,19,20,23-pentaazatetracyclo[13.5.2.1^{2,5}.0^{18,21}]tricosa-1(20),2(23),3,15(22),16, 18(21)-hexaene

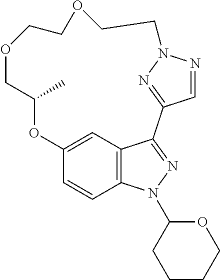 [SMILES: C[C@H]1COCCOCCn2ncc(n2)-c2nn(C3CCCCO3)c3ccc(cc23)O1]

To a suspension of cesium carbonate (511 mg, 1.57 mmol) in anhydrous DMF (130 mL) at 60° C. was added dropwise [(1R)-2-[2-[2-[4-[5-[tert-butyl(dimethyl)silyl]oxy-1-tetrahydro pyran-2-yl-indazol-3-yl]triazol-2-yl]ethoxy]ethoxy]-1-methyl-ethyl]methanesulfonate (326 mg, 0.52 mmol) in DMF (130 mL). The reaction mixture was stirred at 60° C. for 24 h. The reaction mixture was concentrated under reduced pressure, diluted with brine and extracted with ethyl acetate. The combined organic layers were dried over anhydrous sodium sulfate, filtered and evaporated under reduced pressure. The residue was purified by silica gel column chromatography using dichloromethane/ethyl acetate 100/0 to 70/30 as eluent to afford (13S)-13-methyl-19-(oxan-2-yl)-8,11,14-trioxa-4,5,19,20,23-pentaazatetracyclo[13.5.2.1^{2,5}.0^{18,21}] tricosa-1(20),2(23),3,15(22),16,18(21)-hexaene as a white solid.

LCMS method F: [M+H]⁺=414.4, t_{R}=2.60 min

### Preparation of Example 81: (13S)-13-methyl-8,11,14-trioxa-4,5,19,20,23-pentaaza tetracyclo[13.5.2.1¹. 0′-]tricosa-1(20),2(23),3,15(22),16,18(21)-hexaene

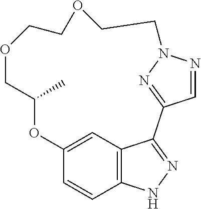 [SMILES: C[C@H]1COCCOCCn2ncc(n2)-c2n[nH]c3ccc(cc23)O1]

To a solution of (13S)-13-methyl-19-(oxan-2-yl)-8,11,14-trioxa-4,5,19,20,23-pentaazatetra cyclo[13.5.2.1^{2,5}.0^{18,21}]tricosa-1(20),2(23),3,15(22),16,18(21)-hexaene (60 mg, 0.15 mmol) in methanol (7 mL) and water (1 mL) was added p-toluenesulfonic acid monohydrate (138 mg, 0.73 mmol). The reaction mixture was stirred at 70° C. for 24 h. The reaction mixture was concentrated under reduced pressure and the residue was neutralized by addition of a saturated aqueous NaHCO₃ solution. The residue was diluted with ethyl acetate, the phases were separated and the aqueous layer was extracted with ethyl acetate. The combined organic layers were washed with brine, dried over anhydrous sodium sulfate, filtered and evaporated under reduced pressure. The residue was recrystallized from acetonitrile, filtered and dried to afford (13S)-13-methyl-8,11,14-trioxa-4,5,19,20,23-pentaazatetracyclo[13.5.2.1^{2,5}.0^{18,21}]tricosa-1(20),2(23),3,15(22),16,18(21)-hexaene as a solid.

LCMS method F: [M+H]⁺=330.3, t_{R}=1.95 min

LCMS method G: [M+H]⁺=330.3, t_{R}=1.93 min

1H NMR (400 MHz, d6-DMSO) 13.08 (1H, s), 8.21 (1H, d, J=2.3 Hz), 8.08 (1H, s), 7.44-7.42 (1H, m), 7.01 (1H, dd, J=2.4, 8.8 Hz), 4.73-4.68 (2H, m), 4.34-4.29 (1H, m), 4.16-4.10 (1H, m), 3.84-3.62 (5H, m), 3.56-3.42 (2H, m), 1.36-1.33 (3H, m) ppm.

### Example 82: (13R)-13-methyl-8,11,14-trioxa-5,19,20-triazatetracyclo[13.5.2.1^{2,5}.0^{18,21}] tricosa-1(20),2(23),3,15(22),16,18(21)-hexaene-4-carbonitrile

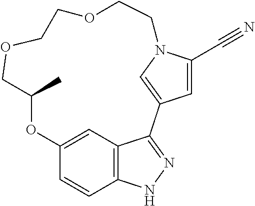 [SMILES: C[C@@H]1COCCOCCn2cc(cc2C#N)-c2n[nH]c3ccc(cc23)O1]

Example 82 is prepared according to the synthesis route described in general Scheme D, following the same synthesis procedures as for Example 72.

### Preparation of Intermediate 480: (13R)-13-methyl-19-(oxan-2-yl)-8,11,14-trioxa-5,19,20-triazatetracyclo[13.5.2.1^{2,5}.0^{18,21}]tricosa-1(20),2(23),3,15(22),16,18(21)-hexaene-4-carbonitrile

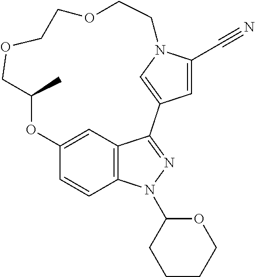 [SMILES: C[C@@H]1COCCOCCn2cc(cc2C#N)-c2nn(C3CCCCO3)c3ccc(cc23)O1]

To a suspension of cesium carbonate (127 mg, 0.39 mmol) in anhydrous DMF (7 mL) at 65° C. was added dropwise [(1S)-2-[2-[2-[4-[5-[tert-butyl(dimethyl)silyl]oxy-1-tetrahydropyran-2-yl-indazol-3-yl]-2-cyano-pyrrol-1-yl]ethoxy]ethoxy]-1-methyl-ethyl] methanesulfonate (83 mg, 0.13 mmol) in DMF (3.5 mL). The reaction mixture was stirred at 65° C. for 16 h. The reaction mixture was cooled to RT, filtered over a pad of Celite and washed with ethyl acetate. The filtrate was concentrated under reduced pressure. The residue was triturated with a mixture of dichloromethane/methanol (9/1), filtered, washed with dichloromethane/methanol (9/1) and dried to afford (13R)-13-methyl-19-(oxan-2-yl)-8,11,14-trioxa-5,19,20-triazatetracyclo[13.5.2.1^{2,5}.0^{18,21}]tricosa-1(20),2(23),3,15(22),16,18(21)-hexaene-4-carbonitrile as a white solid.

LCMS method F: [M+H]⁺=437.2, t_{R}=2.95 min

### Preparation of Example 82: (13R)-13-methyl-8,11,14-trioxa-5,19,20-triazatetracyclo [13.5.2.1^{2,5}.0^{18,21}]tricosa-1(20),2(23),3,15(22),16,18(21)-hexaene-4-carbonitrile

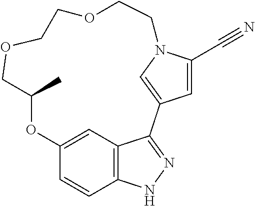 [SMILES: C[C@@H]1COCCOCCn2cc(cc2C#N)-c2n[nH]c3ccc(cc23)O1]

To a solution of (13R)-13-methyl-19-(oxan-2-yl)-8,11,14-trioxa-5,19,20-triazatetracyclo [13.5.2.1^{2,5}.0^{18,21}]tricosa-1(20),2(23),3,15(22),16,18(21)-hexaene-4-carbonitrile (56 mg, 0.13 mmol) in methanol (2.20 mL) and water (0.36 mL) was added p-toluenesulfonic acid monohydrate (123 mg, 0.65 mmol) and the reaction mixture was stirred at 65° C. for 16 h. The reaction mixture was diluted with dichloromethane and saturated aqueous NaHCO₃ solution. The aqueous layer was extracted with dichloromethane. The combined organic layers were washed with brine, dried over anhydrous sodium sulfate, filtered and evaporated under reduced pressure. The residue was purified on preparative TLC on silica gel eluting with dichloromethane/methanol 95/5 as an eluent to afford (13R)-13-methyl-8,11,14-trioxa-5,19,20-triazatetracyclo[13.5.2.1^{2,5}.0^{18,21}]tricosa-1(20),2(23),3,15(22),16,18(21)-hexaene-4-carbonitrile as a solid.

LCMS method F: [M+H]⁺=353.2, t_{R}=2.38 min

LCMS method G: [M+H]⁺=353.3, t_{R}=2.31 min

1H NMR (400 MHz, d6-DMSO) 12.81 (1H, br. s), 7.99 (1H, d, J=1.7 Hz), 7.84 (1H, d, J=1.9 Hz), 7.39 (1H, d, J=8.9 Hz), 7.30 (1H, d, J=1.6 Hz), 6.98 (1H, dd, J=2.1, 8.9 Hz), 4.36-4.24 (3H, m), 3.96-3.90 (1H, m), 3.77-3.68 (5H, m), 3.65-3.59 (1H, m), 3.55-3.50 (1H, m), 1.33 (3H, d, J=6.5 Hz) ppm.

### Example 83: (13R)-13-methyl-8,11,14-trioxa-5,19,20,23-tetraazatetracyclo[13.5.2.1^{2,5}.0^{18,21}]tricosa-1(20),2(23),3,15(22),16,18(21)-hexaene

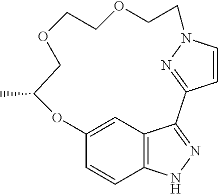 [SMILES: C[C@@H]1COCCOCCn2ccc(n2)-c2n[nH]c3ccc(cc23)O1]

Example 83 is prepared according to the synthesis route described in general Scheme D, following the same synthesis procedures as for Example 80.

### Preparation of Intermediate 481: (13R)-13-methyl-19-(oxan-2-yl)-8,11,14-trioxa-5,19,20,23-tetraazatetracyclo[13.5.2.1^{2,5}.0^{18,21}]tricosa-1(20),2(23),3,15(22),16,18(21)-hexaene

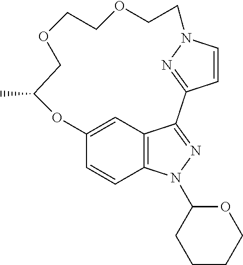 [SMILES: C[C@@H]1COCCOCCn2ccc(n2)-c2nn(C3CCCCO3)c3ccc(cc23)O1]

To a solution of cesium carbonate (262 mg, 0.80 mmol) in dry DMF (15 mL) at 60° C. was added dropwise a solution of [(1S)-2-[2-[2-[3-[5-[tert-butyl(dimethyl)silyl]oxy-1-tetrahydropyran-2-yl-indazol-3-yl]pyrazol-1-yl]ethoxy]ethoxy]-1-methyl-ethyl]methanesulfonate (167 mg, 0.27 mmol) in dry DMF (5 mL). The reaction mixture was stirred at 60° C. for 4 h. The reaction mixture was cooled to RT, filtered over a pad of Celite and washed with ethyl acetate. The solvents were removed under reduced pressure. The residue was purified by silica gel column chromatography using cyclohexane/(ethyl acetate/ethanol (3:1)) 100/0 to 60/40 as eluent. to afford (13R)-13-methyl-19-(oxan-2-yl)-8,11,14-trioxa-5,19,20,23-tetraazatetracyclo[13.5.2.1^{2,5}.0^{18,21}]tricosa-1(20),2(23),3,15(22),16,18(21)-hexaene as a colorless oil.

LCMS method F: [M+H]⁺=413.4, t_{R}=2.51 min

### Preparation of Example 83: (13R)-13-methyl-8,11,14-trioxa-5,19,20,23-tetraazatetra cyclo[13.5.2.1^{2,5}.0^{18,21}]tricosa-1(20),2(23),3,15(22),16,18(21)-hexaene

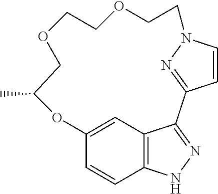 [SMILES: C[C@@H]1COCCOCCn2ccc(n2)-c2n[nH]c3ccc(cc23)O1]

To a solution of (13R)-13-methyl-19-(oxan-2-yl)-8,11,14-trioxa-5,19,20,23-tetraazatetracyclo [13.5.2.1^{2,5}.0^{18,21}]tricosa-1(20),2(23),3,15(22),16,18(21)-hexaene (62 mg, 0.15 mmol) in methanol (4.4 mL) and water (0.7 mL) was added p-toluenesulfonic acid monohydrate (143 mg, 0.75 mmol). The reaction mixture was stirred at 65° C. for 2 h. The reaction mixture was cooled to RT and was quenched with a saturated aqueous NaHCO₃ solution until pH basic. ethyl acetate was added, the phases were separated and the aqueous layer was extracted with ethyl acetate. The combined organic layers were washed with brine, dried over anhydrous sodium sulfate, filtered and evaporated under reduced pressure. The residue was purified by silica gel column chromatography using dichloromethane/methanol 100/0 to 97/3 as eluent to afford (13R)-13-methyl-8,11,14-trioxa-5,19,20,23-tetraazatetracyclo[13.5.2.1^{2,5}.0^{18,21}]tricosa-1(20),2(23),3,15(22),16,18(21)-hexaene as a solid.

LCMS method F: [M+H]⁺=329.3, t_{R}=1.87 min

LCMS method G: [M+H]⁺=329.3, t_{R}=1.86 min

1H NMR (400 MHz, d6-DMSO) 12.80 (1H, s), 8.26 (1H, d, J=2.3 Hz), 7.78 (1H, d, J=2.3 Hz), 7.39-7.36 (1H, m), 6.97 (1H, dd, J=2.5, 8.9 Hz), 6.59 (1H, d, J=2.3 Hz), 4.45-4.27 (3H, m), 4.22-4.16 (1H, m), 3.79-3.49 (6H, m), 3.41 (1H, dd, J=4.2, 9.9 Hz), 1.35 (3H, d, J=6.5 Hz) ppm.

### Example 84: 7,11,14-trioxa-23-thia-4,19,20-triazatetracyclo[13.5.2.1^{2,5}.0^{18,21}]tricosa-1(20), 2,4,15(22), 16,18(21)-hexaene

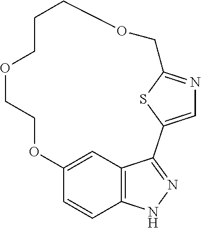 [SMILES: c1cc2[nH]nc3c2cc1OCCOCCCOCc1ncc-3s1]

Example 84 is prepared according to the synthesis route described in general Scheme D.

### Preparation of Intermediate 482: 3-(2-benzyloxyethoxy)propan-1-ol

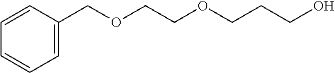 [SMILES: OCCCOCCOCc1ccccc1]

To a solution of propane-1,3-diol (2.07 g, 27.22 mmol) in dry DMF (30 mL) at 0° C. was added sodium hydride (60% dispersion in mineral oil) (1.63 g, 40.83 mmol). The reaction mixture was stirred at 0° C. for 10 min then a solution of 2-benzyloxyethyl 4-methylbenzenesulfonate (intermediate 390) (10 g, 32.67 mmol) in dry DMF (30 mL) was added dropwise. The reaction mixture was allowed to warm up to RT and was stirred at 70° C. overnight. The reaction mixture was cooled to RT and quenched with water. ethyl acetate was added, the phases were separated and the aqueous layer was extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 70/30 as eluent. to afford 3-(2-benzyloxyethoxy)propan-1-ol as a yellow oil.

LCMS method F: [M+H]⁺=211.3, t_{R}=1.87 min

### Preparation of Intermediate 483: 3-(2-benzyloxyethoxy)propyl-4-methylbenzene sulfonate

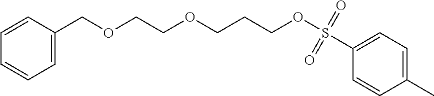 [SMILES: Cc1ccc(S(=O)(=O)OCCCOCCOCc2ccccc2)cc1]

To a cooled solution of 3-(2-benzyloxyethoxy)propan-1-ol (2 g, 9.52 mmol) and triethylamine (2.65 mL, 19.04 mmol) in dry dichloromethane (64 mL) was added p-toluenesulfonyl chloride (1.9 g, 10 mmol). The reaction mixture was stirred at 0° C. for 10 min then at RT for 16 h. The reaction mixture was quenched with a saturated aqueous NaHCO₃ solution and extracted with dichloromethane. The combined organic layers were washed with brine, dried over anhydrous sodium sulfate, filtered and evaporated under reduced pressure. The residue was purified by silica gel column chromatography using cyclohexane/ethyl acetate 100/0 to 70/30 as eluent to afford 3-(2-benzyloxyethoxy)propyl-4-methylbenzenesulfonate as a colorless oil.

LCMS method F: [M+H]⁺=365.3, t_{R}=2.88 min

### Preparation of Intermediate 484: 2-[3-(2-benzyloxyethoxy)propoxymethyl]-5-bromo-thiazole

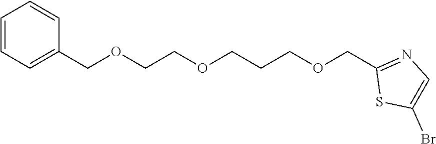 [SMILES: Brc1cnc(COCCCOCCOCc2ccccc2)s1]

To a solution of (5-bromothiazol-2-yl)methanol (1.02 g, 5.33 mmol) in acetonitrile (40 mL) was added cesium carbonate (8.68 g, 26.65 mmol) and 3-(2-benzyloxyethoxy)propyl 4-methylbenzenesulfonate (2.33 g, 6.4 mmol). The reaction mixture was stirred at 70° C. for 48 h. The solvent was evaporated under reduced pressure then water and ethyl acetate were added. The phases were separated and the aqueous layer was extracted with ethyl acetate. The organic layer was dried over anhydrous sodium sulfate, filtered and evaporated under reduced pressure. The residue was purified by silica gel chromatography using cyclohexane/ethyl acetate 100/0 to 70/30 as eluent to afford 2-[3-(2-benzyloxyethoxy)propoxymethyl]-5-bromo-thiazole as an colorless oil.

LCMS method F: [M+H]⁺=386.1-388.1, t_{R}=2.93 min

### Preparation of Intermediate 485: [3-[2-[3-(2-benzyloxyethoxy)propoxymethyl] thiazol-5-yl]-1-tetrahydropyran-2-yl-indazol-5-yl]oxy-tert-butyl-dimethyl-silane

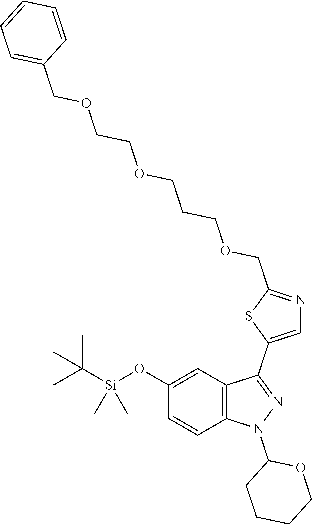 [SMILES: CC(C)(C)[Si](C)(C)Oc1ccc2c(c1)c(-c1cnc(COCCCOCCOCc3ccccc3)s1)nn2C1CCCCO1]

To a degassed solution of tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (977 mg, 2.13 mmol), 2-[3-(2-benzyloxyethoxy)propoxymethyl]-5-bromo-thiazole (822 mg, 2.13 mmol) and potassium phosphate tribasic (1.35 g, 6.39 mmol) in dioxane (3 mL) and water (150 μL) was added tetrakis(triphenylphosphine)palladium(0) (123 mg, 0.106 mmol). The reaction mixture was stirred at 100° C. for 3 h. The solvent was removed under reduced pressure and the residue was diluted with water and ethyl acetate. The phases were separated and the aqueous layer was extracted with ethyl acetate. The organic layers were dried over anhydrous sodium sulfate, filtered and evaporated under reduced pressure. The residue was purified by silica gel column chromatography using cyclohexane/ethyl acetate 100/0 to 50/50 as eluent to afford [3-[2-[3-(2-benzyloxyethoxy)propoxymethyl]thiazol-5-yl]-1-tetrahydropyran-2-yl-indazol-5-yl]oxy-tert-butyl-dimethyl-silane as a orange oil.

LCMS method F: [M+H]⁺=638.4, t_{R}=3.88 min

### Preparation of Intermediate 486: 2-[3-[[5-[5-[tert-butyl(dimethyl)silyl]oxy-1-tetra hydropyran-2-yl-indazol-3-yl]thiazol-2-yl]methoxy]propoxy]ethanol

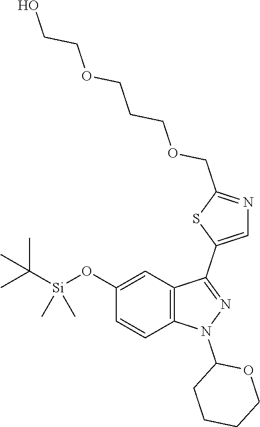 [SMILES: CC(C)(C)[Si](C)(C)Oc1ccc2c(c1)c(-c1cnc(COCCCOCCO)s1)nn2C1CCCCO1]

To a solution of [3-[2-[3-(2-benzyloxyethoxy)propoxymethyl]thiazol-5-yl]-1-tetrahydropyran-2-yl-indazol-5-yl]oxy-tert-butyl-dimethyl-silane (505 mg, 0.793 mmol) in dichloromethane (20 mL) and a pH 7 phosphate buffer (1 mL) at RT was added 2,3-dichloro-5,6-dicyano-p-benzoquinone (450 mg, 1.98 mmol). The reaction mixture was stirred at RT for 19 h. The reaction mixture was diluted with a saturated aqueous NaHCO₃ solution and extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent to afford 2-[3-[[5-[5-[tert-butyl(dimethyl)silyl]oxy-1-tetrahydropyran-2-yl-indazol-3-yl]thiazol-2-yl]methoxy]propoxy]ethanol as an orange oil.

LCMS method F: [M+H]⁺=548.4, t_{R}=3.44 min

### Preparation of Intermediate 487: 2-[3-[[5-[5-[tert-butyl(dimethyl)silyl]oxy-1-tetra hydropyran-2-yl-indazol-3-yl]thiazol-2-yl]methoxy]propoxy]ethyl methanesulfonate

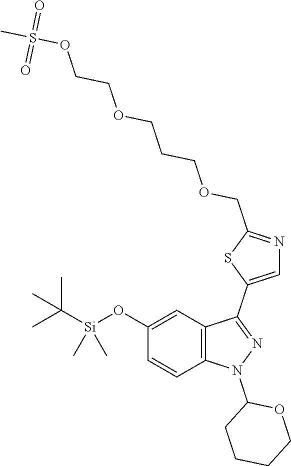 [SMILES: CC(C)(C)[Si](C)(C)Oc1ccc2c(c1)c(-c1cnc(COCCCOCCOS(C)(=O)=O)s1)nn2C1CCCCO1]

To a solution of 2-[3-[[5-[5-[tert-butyl(dimethyl)silyl]oxy-1-tetrahydropyran-2-yl-indazol-3-yl]thiazol-2-yl]methoxy]propoxy]ethanol (240 mg, 0.438 mmol) in dichloromethane (20 mL) at 0° C. was added triethylamine (122 μL, 0.876 mmol) and methanesulfonyl chloride (40 μL, 0.526 mmol). The reaction mixture was stirred at RT overnight. The reaction mixture was diluted with water and extracted with dichloromethane. The combined organic layers were washed with a saturated aqueous ammonium chloride solution and brine, dried over anhydrous sodium sulfate, filtered and evaporated under reduced pressure to afford 2-[3-[[5-[5-[tert-butyl(dimethyl)silyl]oxy-1-tetrahydropyran-2-yl-indazol-3-yl]thiazol-2-yl]methoxy]propoxy] ethylmethanesulfonate as an orange oil which was used in the next step without further purification.

LCMS method F: [M+H]⁺=626.3, t_{R}=3.53 min

### Preparation of Intermediate 488: (13S)-4,13-dimethyl-19-(oxan-2-yl)-7,10,14-trioxa-4,19,20,23-tetraazatetracyclo[13.5.2.1^{2,5}.0^{18,21}]tricosa-1(20),2,5(23),15(22),16,18(21)-hexane

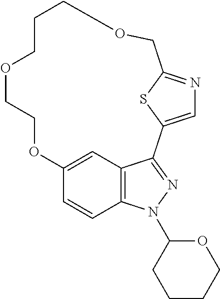 [SMILES: c1cc2c3cc1OCCOCCCOCc1ncc(s1)-c3nn2C1CCCCO1]

To a suspension of cesium carbonate (476 mg, 0.96 mmol) in anhydrous DMF (80 mL) at 60° C. was added dropwise 2-[3-[[5-[5-[tert-butyl(dimethyl)silyl]oxy-1-tetrahydropyran-2-yl-indazol-3-yl]thiazol-2-yl]methoxy]propoxy]ethyl methanesulfonate (200 mg, 0.32 mmol) in DMF (80 mL). The reaction mixture was stirred at 60° C. overnight. The solvent was removed under reduced pressure and the residue was diluted with ethyl acetate and water. The aqueous layer was extracted with ethyl acetate. The organic layers were dried over anhydrous sodium sulfate, filtered and evaporated under reduced pressure. The residue was purified by silica gel column chromatography using cyclohexane/ethyl acetate 100/0 to 50/50 as eluent to afford (13S)-4,13-dimethyl-19-(oxan-2-yl)-7,10,14-trioxa-4,19,20,23-tetraazatetracyclo[13.5.2.1^{2,5}.0^{18,21}]tricosa-1(20),2,5(23),15(22),16,18(21)-hexaene as a yellow oil.

LCMS method F: [M+H]⁺=416.3, t_{R}=2.77 min

### Preparation of Example 84: 7,11,14-trioxa-23-thia-4,19,20-triazatetracyclo[13.5.2.1^{2,5}.0^{18,21}]tricosa-1(20),2,4,15(22), 16,18(21)-hexaene

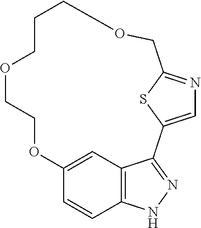 [SMILES: c1cc2[nH]nc3c2cc1OCCOCCCOCc1ncc-3s1]

To a solution of (13S)-4,13-dimethyl-19-(oxan-2-yl)-7,10,14-trioxa-4,19,20,23-tetraazatetra cyclo[13.5.2.1^{2,5}.0^{18,21}]tricosa-1(20),2,5(23),15(22),16,18(21)-hexaene (30 mg, 0.07 mmol) in methanol (5 mL) and water (0.5 mL) was added p-toluenesulfonic acid monohydrate (69 mg, 0.36 mmol) and the reaction mixture was stirred at 65° C. for 2 h. The reaction mixture was concentrated under reduced pressure and the residue was neutralized by addition of saturated aqueous NaHCO₃ solution. Ethyl acetate was added, the phases were separated and the aqueous layer was extracted with ethyl acetate. The combined organic layers was washed with a saturated aqueous NaHCO₃ solution, water and brine then dried over anhydrous sodium sulfate, filtered and evaporated under reduced pressure. The residue was purified by preparative TLC eluting with cyclohexane/ethyl acetate 50/50 as eluent to afford 7,11,14-trioxa-23-thia-4,19,20-triazatetracyclo[13.5.2.1^{2,5}.0^{18,21}]tricosa-1(20),2,4,15(22), 16,18(21)-hexaene as a cream solid.

LCMS method F: [M+H]⁺=332.3, t_{R}=2.11 min

LCMS method G: [M+H]⁺=332.3, t_{R}=2.09 min

1H NMR (400 MHz, d6-DMSO) 13.29 (1H, s), 7.98 (1H, s), 7.56-7.49 (2H, m), 7.04 (1H, dd, J=2.4, 9.0 Hz), 4.90 (2H, s), 4.28 (2H, t, J=5.3 Hz), 3.82-3.65 (6H, m), 1.91-1.85 (2H, m) ppm.

### Example 85: (12S)-12-methyl-8,11,14-trioxa-5,19,20-triazatetracyclo[13.5.2.1^{2,5}.0^{18,21}] tricosa-1(20),2(23),3,15(22),16,18(21)-hexaene-4-carbonitrile

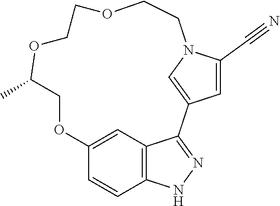 [SMILES: C[C@H]1COc2ccc3[nH]nc(c3c2)-c2cc(C#N)n(c2)CCOCCO1]

Example 85 is prepared according to the synthesis route described in general Scheme D.

### Preparation of Intermediate 489: 2-[2-[(1S)-2-benzyloxy-1-methyl-ethoxy]ethoxy] tetrahydropyran

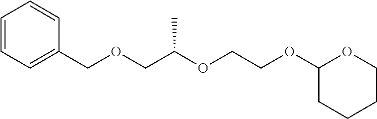 [SMILES: C[C@@H](COCc1ccccc1)OCCOC1CCCCO1]

To a solution of (2S)-1-benzyloxypropan-2-ol intermediate 300 (1.5 g, 9 mmol) in DMF (38 mL) at 0° C. was added sodium hydride (60% dispersion in mineral oil) (900 mg, 13.5 mmol). The reaction mixture was stirred at RT for 1 h then 2-(2-bromoethoxy)tetrahydropyran (4 mL, 27 mmol) was added. The reaction mixture was stirred at RT overnight. The reaction mixture was cooled to 0° C. and additional sodium hydride (60% dispersion in mineral oil) (300 mg, 4.5 mmol) was added. The reaction mixture was stirred at RT for 4 h. The reaction mixture was quenched with water. The phases were separated and the product was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 90/10 as eluent to afford 2-[2-[(1S)-2-benzyloxy-1-methyl-ethoxy]ethoxy]tetrahydropyran (1.76 g, 5.99 mmol) as a pale yellow liquid.

LCMS method F: [M+Na]⁺=317.3, t_{R}=2.71 min

### Preparation of Intermediate 490: 2-[(1S)-2-benzyloxy-1-methyl-ethoxy]ethanol

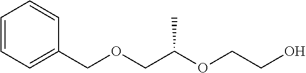

To a solution of 2-[2-[(1S)-2-benzyloxy-1-methyl-ethoxy]ethoxy]tetrahydropyran (1.76 g, 5.99 mmol) in methanol (33 mL) was added p-toluenesulfonic acid monohydrate (114 mg, 0.599 mmol). The reaction mixture was stirred at RT overnight. Methanol was partially removed under reduced pressure and the reaction was quenched with a saturated aqueous NaHCO₃ solution until neutral pH. ethyl acetate was added, the phases were separated and the aqueous layer was extracted with ethyl acetate. The combined organic layers were washed with brine, dried over anhydrous sodium sulfate, filtered and evaporated under reduced pressure to afford 2-[(1S)-2-benzyloxy-1-methyl-ethoxy]ethanol as a pale yellow liquid which was used in the next step without further purification.

LCMS method F: [M+H]⁺=211.2, t_{R}=1.98 min

### Preparation of Intermediate 491: 2-[(1S)-2-benzyloxy-1-methyl-ethoxy]ethylmethane sulfonate

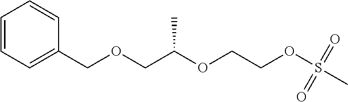 [SMILES: C[C@@H](COCc1ccccc1)OCCOS(C)(=O)=O]

To a solution of 2-[(1S)-2-benzyloxy-1-methyl-ethoxy]ethanol (861 mg, 4.10 mmol) in dichloromethane (25 mL) at 0° C. were added triethylamine (1.14 mL, 8.20 mmol) and methane sulfonyl chloride (0.41 mL, 5.3 mmol). The reaction mixture was stirred at RT for 1 h30. Water and dichloromethane were added and the organic layer was washed with a saturated aqueous solution of NaHCO₃ and with a saturated aqueous solution of NH₄Cl. The organic layer was dried over anhydrous sodium sulfate, filtered and the solvent was removed under reduced pressure to give 2-[(1S)-2-benzyloxy-1-methyl-ethoxy]ethylmethanesulfonate as a yellow liquid. The product was used in the next step without further purification.

LCMS method F: [M+H]⁺=289, t_{R}=2.40 min

### Preparation of Intermediate 492: 1-[2-[2-[(1S)-2-benzyloxy-1-methyl-ethoxy]ethoxy] ethyl]-4-bromo-pyrrole-2-carbonitrile

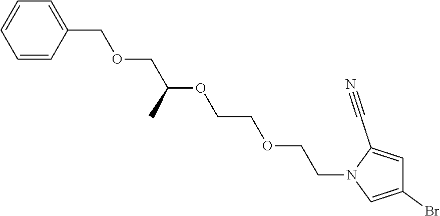 [SMILES: C[C@@H](COCc1ccccc1)OCCOCCn1cc(Br)cc1C#N]

To a solution of 4-bromo-1-(2-hydroxyethyl)pyrrole-2-carbonitrile (intermediate 408) (632 mg, 2.94 mmol) in dry DMF (10 mL) was added sodium hydride (60% dispersion in mineral oil) (353 mg, 8.82 mmol). The reaction mixture was stirred at RT for 30 min then a solution of 2-[(1S)-2-benzyloxy-1-methyl-ethoxy]ethyl methanesulfonate (1.17 g, 4.06 mmol) in dry DMF (6 mL) was added. The reaction mixture was stirred at RT overnight. The reaction mixture was quenched with water and extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford 1-[2-[2-[(1S)-2-benzyloxy-1-methyl-ethoxy]ethoxy]ethyl]-4-bromo-pyrrole-2-carbonitrile as a yellow liquid.

LCMS method F: [M+H]⁺=407-409, t_{R}=3.02 min

### Preparation of Intermediate 493: 1-[2-[2-[(1S)-2-benzyloxy-1-methyl-ethoxy]ethoxy] ethyl]-4-[5-[tert-butyl(dimethyl)silyl]oxy-1-tetrahydropyran-2-yl-indazol-3-yl] pyrrole-2-carbonitrile

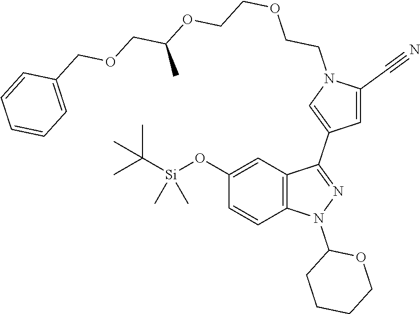 [SMILES: C[C@@H](COCc1ccccc1)OCCOCCn1cc(-c2nn(C3CCCCO3)c3ccc(O[Si](C)(C)C(C)(C)C)cc23)cc1C#N]

To a degassed solution of 1-[2-[2-[(1S)-2-benzyloxy-1-methyl-ethoxy]ethoxy]ethyl]-4-bromo-pyrrole-2-carbonitrile (400 mg, 0.98 mmol) in dioxane (8.6 mL) and water (0.4 mL) was added tert-butyl-dimethyl-[1-tetrahydropyran-2-yl-3-(4,4,5,5-tetramethyl-1,3,2-dioxaborolan-2-yl) indazol-5-yl]oxy-silane (538 mg, 1.18 mmol) and potassium phosphate tribasic (623 mg, 2.94 mmol) then tetrakis(triphenylphosphine)palladium(0) (57 mg, 0.049 mmol) and 2-dicyclohexyl phosphino-2′,4′,6′-triisopropylbiphenyl (47 mg, 0.098 mmol). The reaction mixture was stirred at 110° C. for 2 h. The reaction mixture was filtered through a pad of Celite and washed with ethyl acetate. The filtrate was diluted with water and extracted with ethyl acetate. The organic layer was washed with brine, dried over anhydrous sodium sulfate, filtered and evaporated under reduced pressure. The residue was purified by silica gel column chromatography using cyclohexane/ethyl acetate 100/0 to 80/20 as an eluent to afford 1-[2-[2-[(1S)-2-benzyloxy-1-methyl-ethoxy]ethoxy]ethyl]-4-[5-[tert-butyl(dimethyl)silyl]oxy-1-tetrahydropyran-2-yl-indazol-3-yl]pyrrole-2-carbonitrile as an orange paste.

LCMS method F: [M+H]⁺=659.5, t_{R}=3.81 min

### Preparation of Intermediate 494: 4-[5-[tert-butyl(dimethyl)silyl]oxy-1-tetrahydro pyran-2-yl-indazol-3-yl]-1-[2-[2-[(1S)-2-hydroxy-1-methyl-ethoxy]ethoxy]ethyl] pyrrole-2-carbonitrile

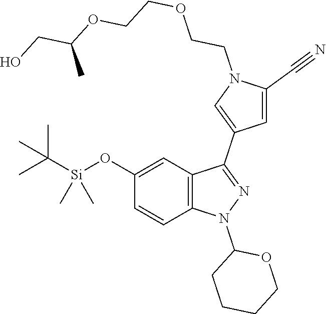 [SMILES: C[C@@H](CO)OCCOCCn1cc(-c2nn(C3CCCCO3)c3ccc(O[Si](C)(C)C(C)(C)C)cc23)cc1C#N]

To a solution of 1-[2-[2-[(1S)-2-benzyloxy-1-methyl-ethoxy]ethoxy]ethyl]-4-[5-[tert-butyl (dimethyl)silyl]oxy-1-tetrahydropyran-2-yl-indazol-3-yl]pyrrole-2-carbonitrile (330 mg, 0.5 mmol) in ethyl acetate (9 mL) at RT was added palladium on carbon 10 wt. % (33 mg). The reaction mixture was stirred under hydrogen atmosphere at RT overnight. Additional palladium on carbon 10 wt. % (33 mg) was added and the reaction mixture was stirred under hydrogen atmosphere at RT for 216 h. The reaction mixture was filtered and the filtrate was removed under reduced pressure. The residue was purified by silica gel column chromatography using cyclohexane/ethyl acetate 100/0 to 50/50 as eluent to afford 4-[5-[tert-butyl(dimethyl) silyl]oxy-1-tetrahydropyran-2-yl-indazol-3-yl]-1-[2-[2-[(1S)-2-hydroxy-1-methyl-ethoxy] ethoxy]ethyl]pyrrole-2-carbonitrile (170 mg, 0.3 mmol) as a yellow oil.

LCMS method F: [M+H]⁺=569.4, t_{R}=3.41 min

### Preparation of Intermediate 495: [(2S)-2-[2-[2-[4-[5-[tert-butyl(dimethyl)silyl]oxy-1-tetrahydropyran-2-yl-indazol-3-yl]-2-cyano-pyrrol-1-yl]ethoxy]ethoxy]propyl] methanesulfonate

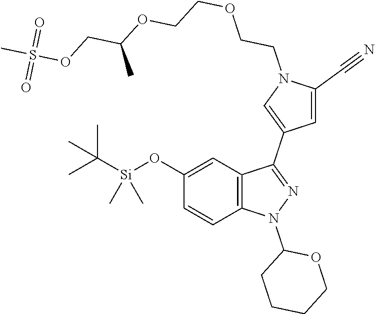 [SMILES: C[C@@H](COS(C)(=O)=O)OCCOCCn1cc(-c2nn(C3CCCCO3)c3ccc(O[Si](C)(C)C(C)(C)C)cc23)cc1C#N]

To a solution of 4-[5-[tert-butyl(dimethyl)silyl]oxy-1-tetrahydropyran-2-yl-indazol-3-yl]-1-[2-[2-[(1S)-2-hydroxy-1-methyl-ethoxy]ethoxy]ethyl]pyrrole-2-carbonitrile (170 mg, 0.3 mmol) in dichloromethane (3.8 mL) at 0° C. was added triethylamine (0.083 mL, 0.6 mol) and methanesulfonyl chloride (0.028 mL, 0.36 mmol). The reaction mixture was stirred at RT for 24 h. The reaction mixture was diluted with water and extracted with dichloromethane. The combined organic layers were washed with a saturated aqueous ammonium chloride solution and brine, dried over anhydrous sodium sulfate, filtered and evaporated under reduced pressure to afford [(2S)-2-[2-[2-[4-[5-[tert-butyl(dimethyl)silyl]oxy-1-tetrahydropyran-2-yl-indazol-3-yl]-2-cyano-pyrrol-1-yl]ethoxy]ethoxy]propyl]methanesulfonate (174 mg, 0.27 mmol) as a yellow viscous oil which was used in the next step without further purification.

LCMS method F: [M+H]⁺=647.3, t_{R}=3.50 min

### Preparation of Intermediate 496: (12S)-12-methyl-19-(oxan-2-yl)-8,11,14-trioxa-5,19,20-triazatetracyclo[13.5.2.1^{2,5}.0^{18,21}]tricosa-1(20),2(23),3,15(22),16,18(21)-hexaene-4-carbonitrile

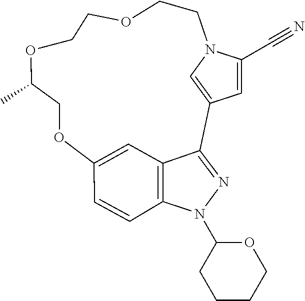 [SMILES: C[C@H]1COc2ccc3c(c2)c(nn3C2CCCCO2)-c2cc(C#N)n(c2)CCOCCO1]

To a suspension of cesium carbonate (263 mg, 0.81 mmol) in anhydrous DMF (12 mL) at 65° C. was added dropwise over a period of 30 min, [(2S)-2-[2-[2-[4-[5-[tert-butyl(dimethyl)silyl]oxy-1-tetrahydropyran-2-yl-indazol-3-yl]-2-cyano-pyrrol-1-yl]ethoxy]ethoxy]propyl]methane sulfonate (174 mg, 0.27 mmol) in DMF (12 mL). The reaction mixture was stirred at 65° C. for 1 h30. The reaction mixture was allowed to cool down to RT, filtered and washed with ethyl acetate. The solvent of the filtrate was removed under reduced pressure. The crude product was purified by column (Macherey Nagel, 15 g) chromatography with cyclohexane/ethyl acetate (100/0 to 70/30) as eluent. The desired fractions were combined and the solvent was removed under reduced pressure to afford (12S)-12-methyl-19-(oxan-2-yl)-8,11,14-trioxa-5,19,20-triaza tetracyclo[13.5.2.1^{2,5}.0^{18,21}]tricosa-1(20),2(23),3,15(22),16,18(21)-hexaene-4-carbonitrile as a white solid.

LCMS method F: [M+H]⁺=437, t_{R}=2.94 min

### Preparation of Example 85: (12S)-12-methyl-8,11,14-trioxa-5,19,20-triazatetracyclo [13.5.2.1^{2,5}.0^{18,21}]tricosa-1(20),2(23),3,15(22),16,18(21)-hexaene-4-carbonitrile

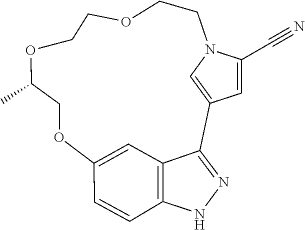 [SMILES: C[C@H]1COc2ccc3[nH]nc(c3c2)-c2cc(C#N)n(c2)CCOCCO1]

To a solution of (12S)-12-methyl-19-(oxan-2-yl)-8,11,14-trioxa-5,19,20-triazatetracyclo [13.5.2.1^{2,5}.0^{18,21}]tricosa-1(20),2(23),3,15(22),16,18(21)-hexaene-4-carbonitrile (95 mg, 0.22 mmol) in methanol (3.7 mL) and water (0.6 mL) was added p-toluenesulfonic acid monohydrate (207 mg, 1.08 mmol). The reaction mixture was stirred at RT overnight. Additional p-toluenesulfonic acid monohydrate (103 mg, 0.54 mmol) was added and the reaction mixture was stirred at RT for 5 h. The reaction mixture was diluted with dichloromethane and a saturated aqueous NaHCO₃ solution. The phases were separated and the aqueous layer was extracted with dichloromethane. The combined organic layers were washed with brine, dried over anhydrous sodium sulfate, filtered and evaporated under reduced pressure. The residue was purified by preparative TLC eluting with cyclohexane/ethyl acetate 50/50 as eluent to afford (12S)-12-methyl-8,11,14-trioxa-5,19,20-triazatetracyclo[13.5.2.1^{2,5}.0^{18,21}]tricosa-1(20),2(23),3, 15(22),16,18(21)-hexaene-4-carbonitrile as a solid.

LCMS method F: [M+H]⁺=353.3, t_{R}=2.30 min

LCMS method G: [M+H]⁺=353.3, t_{R}=2.29 min

1H NMR (400 MHz, CDCl₃) 8.04 (1H, d, J=1.7 Hz), 7.92 (1H, d, J=2 Hz), 7.37 (1H, d, J=8.8 Hz), 7.32 (1H, d, J=1.8 Hz), 7.12 (1H, dd, J=9.1, 2.3 Hz), 4.34 (4H, m), 3.88 (4H, m), 3.79 (1H, m), 3.7 (1H, m), 3.62 (1H, m), 1.36 (3H, d, J=6.3 Hz) ppm.

### Example 86: (12R)-12-methyl-8,11,14-trioxa-5,19,20-triazatetracyclo[13.5.2.1^{2,6}.0^{18,21}] tricosa-1(20),2(23),3,15(22),16,18(21)-hexaene-4-carbonitrile

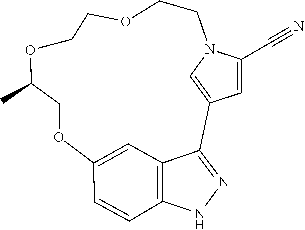 [SMILES: C[C@@H]1COc2ccc3[nH]nc(c3c2)-c2cc(C#N)n(c2)CCOCCO1]

Example 86 is prepared according to the synthesis route described in general Scheme D, following the same synthesis procedures as for Example 85.

### Preparation of Intermediate 497: (12R)-12-methyl-19-(oxan-2-yl)-8,11,14-trioxa-5,19,20-triazatetracyclo[13.5.2.1^{2,5}.0^{18,21}]tricosa-1(20),2(23),3,15(22),16,18(21)-hexaene-4-carbonitrile

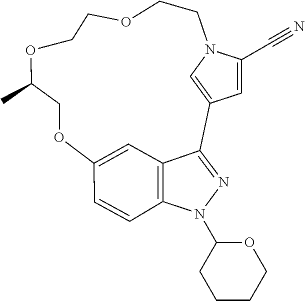 [SMILES: C[C@@H]1COc2ccc3c(c2)c(nn3C2CCCCO2)-c2cc(C#N)n(c2)CCOCCO1]

To a suspension of cesium carbonate (263 mg, 0.81 mmol) in anhydrous DMF (12 mL) at 65° C. was added dropwise [(2R)-2-[2-[2-[4-[5-[tert-butyl(dimethyl)silyl]oxy-1-tetrahydropyran-2-yl-indazol-3-yl]-2-cyano-pyrrol-1-yl]ethoxy]ethoxy]propyl] methanesulfonate (173 mg, 0.27 mmol) in DMF (12 mL). The reaction mixture was stirred at 65° C. for 2 h. The reaction mixture was allowed to cool to RT, filtered and washed with ethyl acetate. The filtrate was removed under reduced pressure. The residue was purified by silica gel column chromatography using cyclohexane/ethyl acetate 100/0 to 70/30 as eluent to afford (12R)-12-methyl-19-(oxan-2-yl)-8,11,14-trioxa-5,19,20-triazatetracyclo[13.5.2.1^{2,5}.0^{18,21}]tricosa-1(20),2(23),3,15(22),16, 18(21)-hexaene-4-carbonitrile as a white solid.

LCMS method F: [M+H]⁺=437.2, t_{R}=2.94 min

### Preparation of Example 86: (12R)-12-methyl-8,11,14-trioxa-5,19,20-triazatetracyclo [13.5.2.1^{2,5}.0^{18,21}]tricosa-1(20),2(23),3,15(22),16,18(21)-hexaene-4-carbonitrile

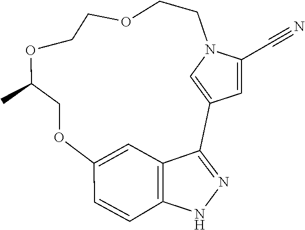 [SMILES: C[C@@H]1COc2ccc3[nH]nc(c3c2)-c2cc(C#N)n(c2)CCOCCO1]

To a solution of (12R)-12-methyl-19-(oxan-2-yl)-8,11,14-trioxa-5,19,20-triazatetracyclo [13.5.2.1^{2,5}.0^{18,21}]tricosa-1(20),2(23),3,15(22),16,18(21)-hexaene-4-carbonitrile (58 mg, 0.13 mmol) in methanol (2.2 mL) and water (0.4 mL) was added p-toluenesulfonic acid monohydrate (126 mg, 0.66 mmol). The reaction mixture was stirred at RT overnight. Additional p-toluenesulfonic acid monohydrate (103 mg, 0.54 mmol) was added and the reaction mixture was stirred at RT for 5 h. The reaction mixture was diluted with dichloromethane and saturated aqueous NaHCO₃ solution. The aqueous layer was extracted with dichloromethane. The combined organic layers were washed with brine, dried over anhydrous sodium sulfate, filtered and evaporated under reduced pressure. The residue was purified by preparative TLC prep eluting with cyclohexane/ethyl acetate 50/50. The resulting solid was triturated with diethyl ether, filtered, washed with diethyl ether and dried to afford (12R)-12-methyl-8,11,14-trioxa-5,19,20-triazatetracyclo[13.5.2.1^{2,5}.0^{18,21}]tricosa-1(20),2(23),3,15(22),16,18(21)-hexaene-4-carbonitrile as a solid.

LCMS method F: [M+H]⁺=353.3, t_{R}=2.30 min

LCMS method G: [M+H]⁺=353.3, t_{R}=2.29 min

1H NMR (400 MHz, CDCl₃) 8.04 (1H, d, J=1.7 Hz), 7.92 (1H, d, J=2 Hz), 7.37 (1H, d, J=9.1 Hz), 7.32 (1H, d, J=1.6 Hz), 7.12 (1H, dd, J=9, 2.2 Hz), 4.34 (4H, m), 3.88 (4H, m), 3.79 (1H, m), 3.7 (1H, m), 3.62 (1H, m), 1.36 (3H, d, J=6.1 Hz) ppm.

### Example 87: (6R,12S)-6,12-dimethyl-8,11,14-trioxa-4,5,19,20-tetraazatetracyclo[13.5.2.1^{2,5}.0^{18,21}]tricosa-1(20),2(23),3,15(22),16,18(21)-hexaene

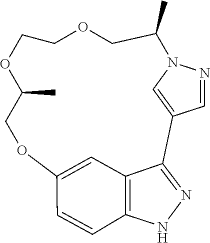 [SMILES: C[C@@H]1COCCO[C@@H](C)COc2ccc3[nH]nc(c3c2)-c2cnn1c2]

Example 87 is prepared according to the synthesis route described in general Scheme D.

### Preparation of Intermediate 498: [(1S)-2-benzyloxy-1-methyl-ethyl]methane sulfonate

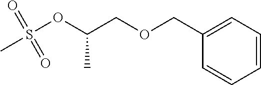 [SMILES: C[C@@H](COCc1ccccc1)OS(C)(=O)=O]

To a solution of (2S)-1-benzyloxypropan-2-ol (intermediate 300) (1 g, 6.02 mmol) in dichloromethane (35 mL) at 0° C. was added triethylamine (1.26 mL, 9.03 mmol) and methanesulfonyl chloride (0.56 mL, 7.22 mmol). The reaction mixture was stirred at 0° C. for 10 min then at RT overnight. Water was added and the phases were separated. The organic layer was washed with a saturated aqueous NaHCO₃ solution and a saturated ammonium chloride solution. The organic layer was dried over anhydrous sodium sulfate, filtered and evaporated under reduced pressure to afford [(1S)-2-benzyloxy-1-methyl-ethyl]methane sulfonate as a pale yellow oil which was used in the next step without further purification.

LCMS method F: [M+H]⁺=245.2, t_{R}=2.28 min

### Preparation of Intermediate 499: 1-[(1R)-2-benzyloxy-1-methyl-ethyl]-4-bromo-pyrazole

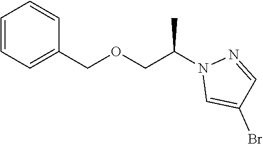 [SMILES: C[C@H](COCc1ccccc1)n1cc(Br)cn1]

To a solution of 4-bromo-1H-pyrazole (707 mg, 4.81 mmol) in ACN (20 mL) were added cesium carbonate (2.037 g, 6.25 mmol) and [(1S)-2-benzyloxy-1-methyl-ethyl]methane sulfonate (1.410 g, 5.77 mmol). The reaction mixture was stirred at 85° C. for 4 h. The reaction was cooled to RT. Water and ethyl acetate were added. The aqueous layer was extracted with ethyl acetate (2×20 mL) and the combined organic layers were dried over anhydrous sodium sulfate, filtered and the solvent was removed under reduced pressure to give 1-[(1R)-2-benzyloxy-1-methyl-ethyl]-4-bromo-pyrazole as a yellow oil. The product was used in the next step without further purification.

LCMS method F: [M+H]⁺=295.0-297.0, t_{R}=2.75 min

### Preparation of Intermediate 500: (2R)-2-(4-bromopyrazol-1-yl)propan-1-ol

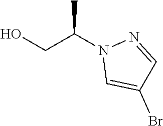

To a solution of 1-[(1R)-2-benzyloxy-1-methyl-ethyl]-4-bromo-pyrazole (1.419 g, 4.81 mmol) in ethanol (30 mL) was added aqueous HCl 37% w. (24 mL) and the resulting mixture was stirred at 80° C. 72 h. The reaction mixture was cooled to RT and the solvent was removed under reduced pressure. The residue was diluted with a saturated aqueous NaHCO₃ solution and ethyl acetate. The aqueous layer was extracted with ethyl acetate and the combined organic layers were dried over anhydrous sodium sulfate, filtered and evaporated under reduced pressure. The residue was purified on silica gel column chromatography using cyclohexane/ethyl acetate 100/0 to 70/30 as eluent to afford (2R)-2-(4-bromopyrazol-1-yl)propan-1-ol as a yellow oil.

LCMS method F: [M+H]⁺=205.1-207.1, t_{R}=1.48 min

### Preparation of Intermediate 501: 1-[(1R)-2-[2-[(1S)-2-benzyloxy-1-methyl-ethoxy] ethoxy]-1-methyl-ethyl]-4-bromo-pyrazole

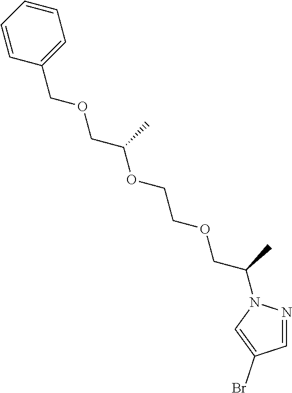 [SMILES: C[C@H](COCCO[C@@H](C)COCc1ccccc1)n1cc(Br)cn1]

To a solution of (2R)-2-(4-bromopyrazol-1-yl)propan-1-ol (250 mg, 1.22 mmol) in dry DMF (3 mL) was added sodium hydride (60% dispersion in mineral oil) (73 mg, 1.83 mmol). The reaction mixture was stirred at RT for 10 min and a solution of 2-[(1S)-2-benzyloxy-1-methyl-ethoxy]ethylmethanesulfonate (intermediate 491) (457 mg, 1.59 mmol) in dry DMF (2 mL) was added. The reaction mixture was stirred at RT for 8 h. Additional sodium hydride (60% dispersion in mineral oil) (73 mg, 1.83 mmol) was added and the resulting mixture was stirred at RT for 72 h. The reaction mixture was quenched with water and ethyl acetate was added. The phases were separated and the aqueous layer was extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 70/30 as eluent to afford 1-[(1R)-2-[2-[(1S)-2-benzyloxy-1-methyl-ethoxy]ethoxy]-1-methyl-ethyl]-4-bromo-pyrazole as a colorless oil.

LCMS method F: [M+H]⁺=397.3-399.3, t_{R}=2.87 min

### Preparation of Intermediate 502: [3-[1-[(1R)-2-[2-[(1S)-2-benzyloxy-1-methyl-ethoxy]ethoxy]-1-methyl-ethyl]pyrazol-4-yl]-1-tetrahydropyran-2-yl-indazol-5-yl] oxy-tert-butyl-dimethyl-silane

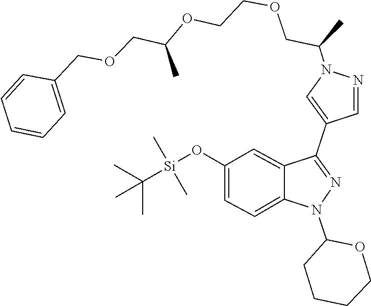 [SMILES: C[C@H](COCCO[C@@H](C)COCc1ccccc1)n1cc(-c2nn(C3CCCCO3)c3ccc(O[Si](C)(C)C(C)(C)C)cc23)cn1]

To a suspension of 1-[(1R)-2-[2-[(1S)-2-benzyloxy-1-methyl-ethoxy]ethoxy]-1-methyl-ethyl]-4-bromo-pyrazole (450 mg, 1.13 mmol) in dioxane (6.2 mL) and water (0.3 mL) was added tert-butyl-dimethyl-[1-tetrahydropyran-2-yl-3-(4,4,5,5-tetramethyl-1,3,2-dioxaborolan-2-yl) indazol-5-yl]oxy-silane (intermediate 61) (673 mg, 1.47 mmol) and potassium phosphate tribasic (720 mg, 3.39 mmol). The reaction mixture was purged with argon for 10 min then tetrakis(triphenylphosphine)palladium(0) (65 mg, 0.06 mmol) and Xphos (54 mg, 0.11 mmol) were added. The reaction mixture was stirred at 90° C. for 4 h. The reaction mixture was cooled to RT and diluted with water and ethyl acetate. The phases were separated then the aqueous layer extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 70/30 as eluent to afford [3-[1-[(1R)-2-[2-[(1S)-2-benzyloxy-1-methyl-ethoxy]ethoxy]-1-methyl-ethyl]pyrazol-4-yl]-1-tetrahydropyran-2-yl-indazol-5-yl]oxy-tert-butyl-dimethyl-silane as a pink oil.

LCMS method F: [M+H]⁺=649.0, t_{R}=3.84 min

### Preparation of Intermediate 503: (2S)-2-[2-[(2R)-2-[4-[5-[tert-butyl(dimethyl)silyl] oxy-1-tetrahydropyran-2-yl-indazol-3-yl]pyrazol-1-yl]propoxy]ethoxy]propan-1-ol

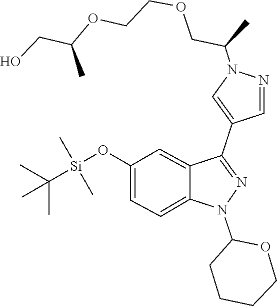 [SMILES: C[C@H](COCCO[C@@H](C)CO)n1cc(-c2nn(C3CCCCO3)c3ccc(O[Si](C)(C)C(C)(C)C)cc23)cn1]

To a suspension of [3-[1-[(1R)-2-[2-[(1S)-2-benzyloxy-1-methyl-ethoxy]ethoxy]-1-methyl-ethyl]pyrazol-4-yl]-1-tetrahydropyran-2-yl-indazol-5-yl]oxy-tert-butyl-dimethyl-silane (350 mg, 0.54 mmol) in ethanol (4.5 mL) under argon was added palladium on charcoal 10% (35 mg). The reaction mixture was stirred under hydrogen atmosphere at RT overnight. The reaction mixture was filtered over a pad of Celite and washed with ethanol and ethyl acetate. The filtrate was removed under reduced pressure to give (2S)-2-[2-[(2R)-2-[4-[5-[tert-butyl(dimethyl)silyl]oxy-1-tetrahydropyran-2-yl-indazol-3-yl]pyrazol-1-yl]propoxy]ethoxy] propan-1-ol as a brown oil which was used in the next step without further purification.

LCMS method F: [M+H]⁺=559.5, t_{R}=3.31 min

### Preparation of Intermediate 504: [(2S)-2-[2-[(2R)-2-[4-[5-[tert-butyl(dimethyl)silyl] oxy-1-tetrahydropyran-2-yl-indazol-3-yl]pyrazol-1-yl]propoxy]ethoxy]propyl] methanesulfonate

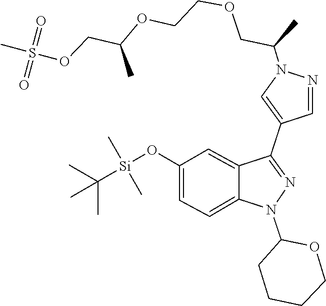 [SMILES: C[C@H](COCCO[C@@H](C)COS(C)(=O)=O)n1cc(-c2nn(C3CCCCO3)c3ccc(O[Si](C)(C)C(C)(C)C)cc23)cn1]

To a solution of (2S)-2-[2-[(2R)-2-[4-[5-[tert-butyl(dimethyl)silyl]oxy-1-tetrahydropyran-2-yl-indazol-3-yl]pyrazol-1-yl]propoxy]ethoxy]propan-1-ol (237 mg, 0.42 mmol) in dichloro methane (2.5 mL) at 0° C. was added triethylamine (0.12 mL, 0.84 mmol) and methanesulfonyl chloride (0.04 mL, 0.55 mmol). The reaction mixture was stirred at 0° C. for 10 min then RT for 1 h. Water and dichloromethane were added. The phases were separated and the organic layer was washed with a saturated aqueous NaHCO₃ solution and saturated ammonium chloride solution. The organic layer was dried over anhydrous sodium sulfate, filtered and evaporated under reduced pressure to afford [(2S)-2-[2-[(2R)-2-[4-[5-[tert-butyl(dimethyl)silyl]oxy-1-tetra hydropyran-2-yl-indazol-3-yl]pyrazol-1-yl]propoxy]ethoxy]propyl]methanesulfonate as an orange oil which was used in the next step without further purification.

LCMS method F: [M+H]⁺=637.5, t_{R}=3.45 min

### Preparation of Intermediate 505: (6R,12S)-6,12-dimethyl-19-(oxan-2-yl)-8,11,14-trioxa-4,5,19,20-tetraazatetracyclo[13.5.2.1^{2,5}.0^{18,21}]tricosa-1(20),2(23),3,15(22),16, 18(21)-hexaene

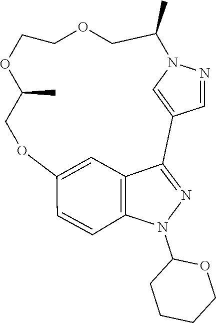 [SMILES: C[C@@H]1COCCO[C@@H](C)COc2ccc3c(c2)c(nn3C2CCCCO2)-c2cnn1c2]

To a solution of cesium carbonate (414 mg, 1.27 mmol) in dry DMF (8 mL) at 60° C. was added dropwise a solution of [(2S)-2-[2-[(2R)-2-[4-[5-[tert-butyl(dimethyl)silyl]oxy-1-tetrahydro pyran-2-yl-indazol-3-yl]pyrazol-1-yl]propoxy]ethoxy]propyl] methanesulfonate (270 mg, 0.42 mmol) in dry DMF (3 mL). The reaction mixture was stirred at 60° C. for 4 h. The reaction mixture was cooled to RT, filtered over a pad of Celite and washed with ethyl acetate. The filtrate was removed under reduced pressure. The residue was purified by silica gel column chromatography using cyclohexane/(ethyl acetate/ethanol (3/1)) 100/0 to 60/40 as eluent. to afford (6R,12S)-6,12-dimethyl-19-(oxan-2-yl)-8,11,14-trioxa-4,5,19,20-tetraazatetracyclo [13.5.2.1^{2,5}.0^{18,21}]tricosa-1(20),2(23),3,15(22),16,18(21)-hexaene as a white solid.

LCMS method F: [M+H]⁺=427.4, t_{R}=2.66 min

### Preparation of Example 87: (6R,12S)-6,12-dimethyl-8,11,14-trioxa-4,5,19,20-tetraazatetracyclo[13.5.2.1^{2,5}.0^{18,21}]tricosa-1(20),2(23),3,15(22),16,18(21)-hexaene

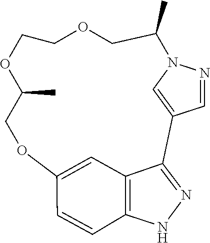 [SMILES: C[C@@H]1COCCO[C@@H](C)COc2ccc3[nH]nc(c3c2)-c2cnn1c2]

To a solution of (6R,12S)-6,12-dimethyl-19-(oxan-2-yl)-8,11,14-trioxa-4,5,19,20-tetraaza tetra cyclo[13.5.2.1^{2,5}.0^{18,21}]tricosa-1(20),2(23),3,15(22),16,18(21)-hexaene (63 mg, 0.15 mmol) in methanol (4.4 mL) and water (0.7 mL) was added p-toluenesulfonic acid monohydrate (140 mg, 0.74 mmol). The reaction mixture was stirred at 65° C. for 2 h. The reaction mixture was cooled to RT and quenched with a saturated aqueous NaHCO₃ solution until pH basic. Ethyl acetate was added and the phases were separated. The aqueous layer was extracted with ethyl acetate and the combined organic layers were washed with brine, dried over anhydrous sodium sulfate, filtered and evaporated under reduced pressure. The residue was purified by silica gel column chromatography using dichloromethane/methanol 100/0 to 97/3 as eluent to afford (6R,12S)-6,12-dimethyl-8,11,14-trioxa-4,5,19,20-tetraazatetracyclo[13.5.2.1^{2,5}.0^{18,21}]tricosa-1(20),2(23),3,15(22),16,18(21)-hexaene as a solid.

LCMS method F: [M+H]⁺=343.3, t_{R}=2.07 min

LCMS method G: [M+H]⁺=343.3, t_{R}=2.06 min

1H NMR (400 MHz, d6-DMSO) 12.73 (1H, s), 8.47-8.46 (1H, m), 7.80-7.78 (2H, m), 7.39-7.36 (1H, m), 7.01 (1H, dd, J=2.3, 8.9 Hz), 4.65-4.59 (1H, m), 4.29-4.17 (2H, m), 3.85-3.59 (7H, m), 1.51 (3H, d, J=6.8 Hz), 1.21 (3H, d, J=6.4 Hz) ppm.

### Example 88: (13R)-17-fluoro-13-methyl-8,11,14-trioxa-4,5,19,20-tetraazatetracyclo [13.5.2.1^{2,5}.0^{18,21}]tricosa-1(20),2(23),3,15(22),16,18(21)-hexaene

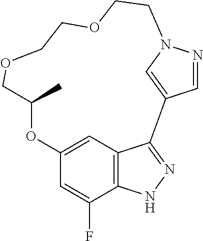 [SMILES: C[C@@H]1COCCOCCn2cc(cn2)-c2n[nH]c3c(F)cc(cc23)O1]

Example 88 is prepared according to the synthesis route described in general Scheme B.

### Preparation of Intermediate 506: 2-[2-[2-[(2S)-2-benzyloxypropoxy]ethoxy]ethoxy] tetrahydropyran

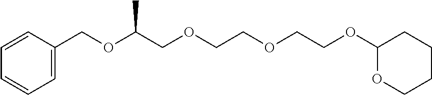 [SMILES: C[C@@H](COCCOCCOC1CCCCO1)OCc1ccccc1]

2-[(2S)-2-benzyloxypropoxy]ethyl methanesulfonate was prepared according to the same procedure as for 2-[(2R)-2-benzyloxypropoxy]ethylmethanesulfonate (intermediate 387), starting from methyl (2S)-2-benzyloxypropanoate.

To a solution of 2-tetrahydropyran-2-yloxyethanol (700 μL, 5.16 mmol) in dry DMF (12 mL) at 0° C. was added sodium hydride (60% dispersion in mineral oil) (275 mg, 6.88 mmol). The mixture was stirred at RT for 10 min and a solution of 2-[(2S)-2-benzyloxypropoxy]ethyl methanesulfonate (1.24 g, 3.44 mmol) in dry DMF (2 mL) was added. The reaction mixture was stirred at RT overnight. Additional 2-tetrahydropyran-2-yloxyethanol (0.14 mL, 1.03 mmol) and sodium hydride (60% dispersion in mineral oil) (41 mg, 1.03 mmol) were added and the reaction mixture was stirred at RT for 4 h. The reaction mixture was quenched with water and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 80/20 as eluent to afford 2-[2-[2-[(2S)-2-benzyloxypropoxy]ethoxy]ethoxy]tetrahydropyran (1.13 g, 3.34 mmol) as a pale yellow liquid.

LCMS method F: [M+Na]⁺=361.2, t_{R}=2.70 min

### Preparation of Intermediate 507: 2-[2-[(2S)-2-benzyloxypropoxy]ethoxy]ethanol

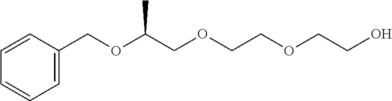 [SMILES: C[C@@H](COCCOCCO)OCc1ccccc1]

To a solution of 2-[2-[2-[(2S)-2-benzyloxypropoxy]ethoxy]ethoxy]tetrahydropyran (1.13 g, 3.34 mmol) in methanol (14 mL) was added p-toluenesulfonic acid monohydrate (63 mg, 0.334 mmol). The reaction mixture was stirred at RT overnight. Methanol was partially removed under reduced pressure and the reaction mixture was quenched with a saturated aqueous NaHCO₃ solution until pH neutral. Ethyl acetate was added, the phases were separated and the aqueous layer was extracted with ethyl acetate. The combined organic layers were washed with brine, dried over anhydrous sodium sulfate, filtered and evaporated under reduced pressure to afford 2-[2-[(2S)-2-benzyloxypropoxy]ethoxy]ethanol as a pale yellow liquid which was used in the next step without further purification.

LCMS method F: [M+H]⁺=255.3, t_{R}=1.92 min

### Preparation of Intermediate 508: 2-[2-[(2S)-2-benzyloxypropoxy]ethoxy]ethyl methanesulfonate

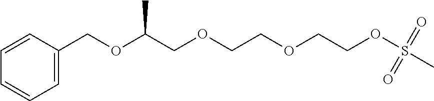 [SMILES: C[C@@H](COCCOCCOS(C)(=O)=O)OCc1ccccc1]

To a suspension 2-[2-[(2S)-2-benzyloxypropoxy]ethoxy]ethanol (690 mg, 2.72 mmol) in dichloromethane (12 mL) at 0° C. was added triethylamine (0.57 mL, 4.08 mmol) and methanesulfonyl chloride (0.25 mL, 3.26 mmol). The reaction mixture was stirred at RT for 3 h. The mixture was diluted with water and dichloromethane. The phases were separated and the organic layer was washed with a saturated aqueous NaHCO₃ solution and saturated aqueous ammonium chloride solution, dried over anhydrous sodium sulfate, filtered and evaporated under reduced pressure to afford 2-[2-[(2S)-2-benzyloxypropoxy]ethoxy]ethyl methane sulfonate as a yellow liquid which was used in the next step without further purification.

LCMS method F: [M+H]⁺=333.1, t_{R}=2.38 min

### Preparation of Intermediate 509: 1-[2-[2-[(2S)-2-benzyloxypropoxy]ethoxy]ethyl]-4-(4,4,5,5-tetramethyl-1,3,2-dioxaborolan-2-yl)pyrazole

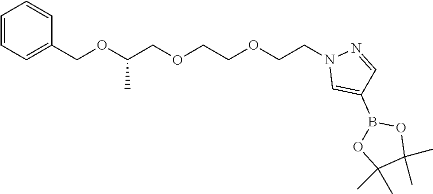 [SMILES: C[C@@H](COCCOCCn1cc(B2OC(C)(C)C(C)(C)O2)cn1)OCc1ccccc1]

To a solution of 2-[2-[(2S)-2-benzyloxypropoxy]ethoxy]ethyl methanesulfonate (873 mg, 2.63 mmol) and 4-(4,4,5,5-tetramethyl-1,3,2-dioxaborolan-2-yl)-1H-pyrazole (392 mg, 2.02 mmol) in acetonitrile (20 mL) at RT was added cesium carbonate (1.07 g, 3.3 mmol). The reaction mixture was stirred at 85° C. overnight. The reaction mixture was filtrated and the filtrate was removed under reduced pressure. The residue was dissolved in ethyl acetate and water was added. The phases were separated and the aqueous layer was extracted with ethyl acetate. The combined organic layers were washed with brine, dried over anhydrous sodium sulfate, filtered and evaporated under reduced pressure to afford 1-[2-[2-[(2S)-2-benzyloxypropoxy]ethoxy] ethyl]-4-(4,4,5,5-tetramethyl-1,3,2-dioxaborolan-2-yl)pyrazole as a yellow oil which was used in the next step without further purification.

LCMS method F: [M+H]⁺=431.3, t_{R}=2.81 min

### Preparation of Intermediate 510: tert-butyl-[(7-fluoro-1H-indazol-5-yl)oxy]-dimethyl-silane

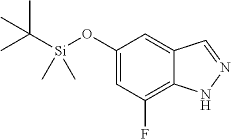 [SMILES: CC(C)(C)[Si](C)(C)Oc1cc(F)c2[nH]ncc2c1]

To a solution of 7-fluoro-1H-indazol-5-ol (4.85 g, 31.93 mmol) in dichloromethane (100 mL) was added imidazole (2.609 g, 38.32 mmol) and tert-butyldimethylchlorosilane (5.29 g, 35.13 mmol). The reaction mixture was stirred at RT for 16 h. The reaction mixture was filtered on a pad of Celite and was washed with dichloromethane. The filtrate was removed under reduced pressure to afford tert-butyl-[(7-fluoro-1H-indazol-5-yl)oxy]-dimethyl-silane which was used in the next step without further purification.

LCMS method B: [M+H]⁺=267.0, t_{R}=1.153 min

### Preparation of Intermediate 511: tert-butyl-[(7-fluoro-3-iodo-1H-indazol-5-yl)oxy]-dimethyl-silane

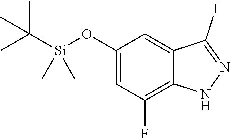 [SMILES: CC(C)(C)[Si](C)(C)Oc1cc(F)c2[nH]nc(I)c2c1]

To a solution of tert-butyl-[(7-fluoro-1H-indazol-5-yl)oxy]-dimethyl-silane (6 g, 22.52 mmol) in DMF (15 mL) was added 1-iodopyrrolidine-2,5-dione (7.601 g, 33.785 mmol) in DMF (30 mL). The reaction mixture was stirred at RT for 1 h. 10% sodium thiosulfate solution was added at 0° C. and the mixture was extracted with ethyl acetate. The combined organic layers were dried over anhydrous magnesium sulfate, filtered and evaporated under reduced pressure. The residue was purified by silica gel column chromatography using as eluent heptane/ethyl acetate 90/10 as eluent to afford of tert-butyl-[(7-fluoro-3-iodo-1H-indazol-5-yl)oxy]-dimethyl-silane as a sticky transparent gum.

LCMS method B: [M+H]⁺=392.9, t_{R}=1.316 min

### Preparation of Intermediate 512: tert-butyl-(7-fluoro-3-iodo-1-tetrahydropyran-2-yl-indazol-5-yl)oxy-dimethyl-silane

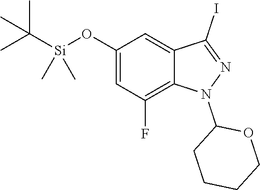 [SMILES: CC(C)(C)[Si](C)(C)Oc1cc(F)c2c(c1)c(I)nn2C1CCCCO1]

To a solution of tert-butyl-[(7-fluoro-3-iodo-1H-indazol-5-yl)oxy]-dimethyl-silane (5.34 g, 13.61 mmol) in dichloromethane (54 mL) was added 4-methylbenzenesulfonic acid monohydrate (518 mg, 2.72 mmol) and 3,4-dihydro-2H-pyran (3.73 ml, 40.83 mmol). The reaction mixture was stirred at RT for 16 h. The reaction mixture was diluted with dichloromethane and washed with a saturated aqueous NaHCO₃ solution and brine. The organic layer was dried over anhydrous magnesium sulfate, filtered and evaporated under reduced pressure. The residue was purified by silica gel column chromatography using heptane/ethyl acetate 100/0 to 90/10 as eluent to afford tert-butyl-(7-fluoro-3-iodo-1-tetrahydropyran-2-yl-indazol-5-yl)oxy-dimethyl-silane as a colorless oil.

LCMS method B: [M−THP]⁺=392.9, t_{R}=1.577 min

### Preparation of Intermediate 514: [3-[1-[2-[2-[(2S)-2-benzyloxypropoxy]ethoxy]ethyl] pyrazol-4-yl]-7-fluoro-1-tetrahydropyran-2-yl-indazol-5-yl]oxy-tert-butyl-dimethyl-silane

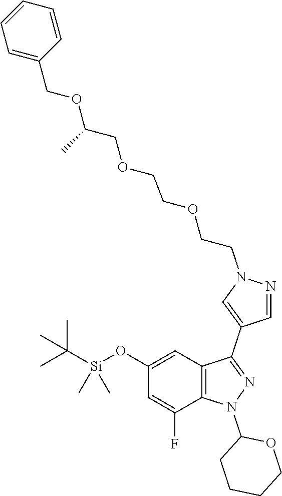 [SMILES: C[C@@H](COCCOCCn1cc(-c2nn(C3CCCCO3)c3c(F)cc(O[Si](C)(C)C(C)(C)C)cc23)cn1)OCc1ccccc1]

To a degassed solution of tert-butyl-(7-fluoro-3-iodo-1-tetrahydropyran-2-yl-indazol-5-yl)oxy-dimethyl-silane (553 mg, 1.16 mmol), 1-[2-[2-[(2S)-2-benzyloxypropoxy]ethoxy]ethyl]-4-(4,4,5,5-tetramethyl-1,3,2-dioxaborolan-2-yl)pyrazole (intermediate 509) (956 mg, 1.95 mmol) and potassium phosphate tribasic (737 mg, 3.48 mmol) in dioxane (32 mL) and water (1.5 mL) were added tetrakis(triphenylphosphine)palladium(0) (67 mg, 0.058 mmol) and 2-dicyclohexylphosphino-2′,4′,6′-triisopropylbiphenyl (55 mg, 0.116 mmol). The reaction was stirred at 110° C. overnight. The reaction mixture was filtered over a pad of Celite and the filtrate was diluted with water and ethyl acetate. The aqueous layer was extracted with ethyl acetate. The combined organic layers were washed with brine, dried over anhydrous sodium sulfate and evaporated under reduced pressure. The residue was purified by silica gel column chromatography using cyclohexane/ethyl acetate 100/0 to 60/40 as eluent to afford [3-[1-[2-[2-[(2S)-2-benzyloxypropoxy]ethoxy]ethyl]pyrazol-4-yl]-7-fluoro-1-tetrahydropyran-2-yl-indazol-5-yl]oxy-tert-butyl-dimethyl-silane as a colorless viscous oil.

LCMS method J: [M+H]⁺=653.4, t_{R}=6.00 min

### Preparation of Intermediate 515: (2S)-1-[2-[2-[4-[5-[tert-butyl(dimethyl)silyl]oxy-7-fluoro-1-tetrahydropyran-2-yl-indazol-3-yl]pyrazol-1-yl]ethoxy]ethoxy]propan-2-ol

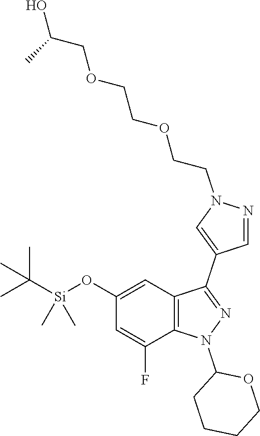 [SMILES: C[C@H](O)COCCOCCn1cc(-c2nn(C3CCCCO3)c3c(F)cc(O[Si](C)(C)C(C)(C)C)cc23)cn1]

To a solution of [3-[1-[2-[2-[(2S)-2-benzyloxypropoxy]ethoxy]ethyl]pyrazol-4-yl]-7-fluoro-1-tetrahydropyran-2-yl-indazol-5-yl]oxy-tert-butyl-dimethyl-silane (326 mg, 0.5 mmol) in ethyl acetate (9 mL) at RT was added palladium on carbon 10 wt. % (33 mg). The reaction mixture was stirred under hydrogen atmosphere at RT for 72 h. The reaction mixture filtered and the filtrate was concentrated under reduced pressure. The residue was purified by silica gel column chromatography using cyclohexane/ethyl acetate 100/0 to 0/100 as eluent to afford (2S)-1-[2-[2-[4-[5-[tert-butyl(dimethyl)silyl]oxy-7-fluoro-1-tetrahydropyran-2-yl-indazol-3-yl]pyrazol-1-yl]ethoxy]ethoxy]propan-2-ol as a colorless oil.

LCMS method F: [M+H]⁺=563.3, t_{R}=3.34 min

### Preparation of Intermediate 516: [(1S)-2-[2-[2-[4-[5-[tert-butyl(dimethyl)silyl]oxy-7-fluoro-1-tetrahydropyran-2-yl-indazol-3-yl]pyrazol-1-yl]ethoxy]ethoxy]-1-methyl-ethyl]methanesulfonate

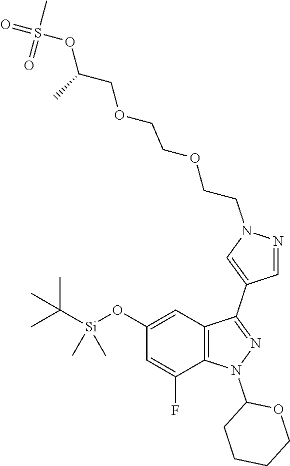 [SMILES: C[C@@H](COCCOCCn1cc(-c2nn(C3CCCCO3)c3c(F)cc(O[Si](C)(C)C(C)(C)C)cc23)cn1)OS(C)(=O)=O]

To a solution of (2S)-1-[2-[2-[4-[5-[tert-butyl(dimethyl)silyl]oxy-7-fluoro-1-tetrahydropyran-2-yl-indazol-3-yl]pyrazol-1-yl]ethoxy]ethoxy]propan-2-ol (124 mg, 0.22 mmol) in dichloro methane (2.8 mL) at 0° C. was added triethylamine (0.06 mL, 0.44 mmol) and methanesulfonyl chloride (0.02 mL, 0.26 mmol). The reaction mixture was stirred at RT overnight. Additional triethylamine (0.012 mL, 0.088 mmol) and methanesulfonyl chloride (0.003 mL, 0.044 mmol) were added and the reaction mixture was stirred at RT for 4 h. Additional triethylamine (0.012 mL, 0.088 mmol) and methanesulfonyl chloride (0.003 mL, 0.044 mmol) were added and the reaction mixture was stirred at RT for 1 h. The reaction mixture was diluted with water and extracted with dichloromethane. The combined organic layers were washed with a saturated ammonium chloride solution and brine, dried over anhydrous sodium sulfate, filtered and evaporated under reduced pressure to afford [(1S)-2-[2-[2-[4-[5-[tert-butyl(dimethyl)silyl]oxy-7-fluoro-1-tetrahydropyran-2-yl-indazol-3-yl]pyrazol-1-yl]ethoxy]ethoxy]-1-methyl-ethyl] methanesulfonate as an orange oil which was used in the next step without further purification.

LCMS method F: [M+H]⁺=641.4, t_{R}=3.51 min

### Preparation of Intermediate 517: (13R)-17-fluoro-13-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16, 18(21)-hexaene

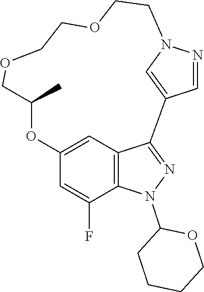 [SMILES: C[C@@H]1COCCOCCn2cc(cn2)-c2nn(C3CCCCO3)c3c(F)cc(cc23)O1]

To a suspension of cesium carbonate (190 mg, 0.585 mmol) in anhydrous DMF (9 mL) at 65° C. was added dropwise [(1S)-2-[2-[2-[4-[5-[tert-butyl(dimethyl)silyl]oxy-7-fluoro-1-tetrahydro pyran-2-yl-indazol-3-yl]pyrazol-1-yl]ethoxy]ethoxy]-1-methyl-ethyl]methanesulfonate (125 mg, 0.195 mmol) in DMF (9 mL). The reaction mixture was stirred at 65° C. for 1 h. The reaction mixture was allowed to cool to RT, filtered and washed with ethyl acetate. The filtrate was removed under reduced pressure. The residue was purified by silica gel column chromatography using dichloromethane/methanol 100/0 to 98/2 as eluent to afford (13R)-17-fluoro-13-methyl-19-(oxan-2-yl)-8,11,14-trioxa-4,5,19,20-tetraazatetracyclo [13.5.2.1^{2,5}.0^{18,21}] tricosa-1(20),2(23),3,15(22),16,18(21)-hexaene as a white solid.

LCMS method F: [M+H]⁺=431.4, t_{R}=2.79 min

### Preparation of Example 88: (13R)-17-fluoro-13-methyl-8,11,14-trioxa-4,5,19,20-tetraazatetracyclo[13.5.2.1^{2,5}. 0^{18,21}]tricosa-1(20),2(23),3,15(22),16,18(21)-hexaene

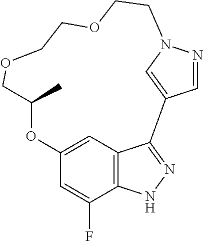 [SMILES: C[C@@H]1COCCOCCn2cc(cn2)-c2n[nH]c3c(F)cc(cc23)O1]

To a solution of (13R)-17-fluoro-13-methyl-19-(oxan-2-yl)-8,11,14-trioxa-4,5,19,20-tetraaza tetracyclo[13.5.2.1^{2,5}.0^{18,21}]tricosa-1(20),2(23),3,15(22),16,18(21)-hexaene (25 mg, 0.058 mmol) in methanol (1 mL) and water (0.17 mL) was added p-toluenesulfonic acid monohydrate (55 mg, 0.29 mmol). The reaction mixture was stirred at 65° C. overnight. The reaction mixture was diluted with dichloromethane and saturated aqueous NaHCO₃ solution. The aqueous layer was extracted with dichloromethane. The combined organic layers were washed with brine, dried over anhydrous sodium sulfate, filtered and evaporated under reduced pressure. The residue was triturated with diethyl ether, filtered and washed with diethyl ether and dried to afford (13R)-17-fluoro-13-methyl-8,11,14-trioxa-4,5,19,20-tetraazatetracyclo[13.5.2.1^{2,5}.0^{18,21}] tricosa-1(20),2(23),3,15(22),16,18(21)-hexaene as a powder.

LCMS method F: [M+H]⁺=347.3, t_{R}=2.13 min

LCMS method G: [M+H]⁺=347.3, t_{R}=2.13 min

1H NMR (400 MHz, CDCl₃) 8.53 (1H, d, J=0.6 Hz), 8.01 (1H, d, J=0.7 Hz), 7.79 (1H, d, J=1.7 Hz), 6.83 (1H, dd, J=1.9, 11.8 Hz), 4.49 (3H, m), 4.03 (1H, m), 3.87 (1H, dd, J=10.2, 3.7 Hz), 3.73 (5H, m), 3.58 (1H, dd, J=10.2, 2.4 Hz), 1.44 (3H, d, J=6.8 Hz) ppm.

### Example 89: (6S,12S)-6,12-dimethyl-8,11,14-trioxa-4,5,19,20-tetraazatetracyclo [13.5.2.1^{2,5}.0^{18,21}]tricosa-1(20),2(23),3,15(22),16,18(21)-hexaene

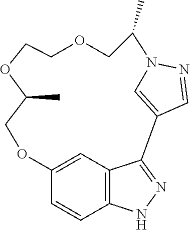 [SMILES: C[C@H]1COc2ccc3[nH]nc(c3c2)-c2cnn(c2)[C@@H](C)COCCO1]

Example 89 is prepared according to the synthesis route described in general Scheme D, following the same synthesis procedures as for Example 87.

### Preparation of Intermediate 518: (6S,12S)-6,12-dimethyl-19-(oxan-2-yl)-8,11,14-trioxa-4,5,19,20-tetraazatetracyclo[13.5.2.1^{2,5}.0^{18,21}]tricosa-1(20),2(23),3,15(22), 16,18(21)-hexaene

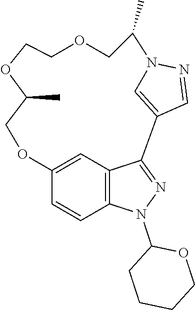 [SMILES: C[C@H]1COc2ccc3c(c2)c(nn3C2CCCCO2)-c2cnn(c2)[C@@H](C)COCCO1]

To a solution of cesium carbonate (539 mg, 1.66 mmol) in dry DMF (40 mL) at 60° C. was added dropwise a solution of [(2S)-2-[2-[(2S)-2-[4-[5-[tert-butyl(dimethyl)silyl]oxy-1-tetrahydro pyran-2-yl-indazol-3-yl]pyrazol-1-yl]propoxy]ethoxy]propyl] methanesulfonate (352 mg, 0.55 mmol) in dry DMF (10 mL). The reaction mixture was stirred at 60° C. for 4 h. The mixture was cooled to RT, filtered over a pad of Celite and washed with ethyl acetate. The filtrate was evaporated under reduced pressure. The residue was purified by silica gel column chromatography using cyclohexane/ethyl acetate 100/0 to 50/50 as eluent to afford (6S,12S)-6,12-dimethyl-19-(oxan-2-yl)-8,11,14-trioxa-4,5,19,20-tetraazatetracyclo[13.5.2.1^{2,5}.0^{18,21}] tricosa-1(20),2(23),3,15(22),16,18(21)-hexaene as a white solid.

LCMS method F: [M+H]⁺=427.2, t_{R}=2.69 min

### Preparation of Example 89: (6S,12S)-6,12-dimethyl-8,11,14-trioxa-4,5,19,20-tetraazatetracyclo[13.5.2.1^{2,5}.0^{18,21}]tricosa-1(20),2(23),3,15(22),16,18(21)-hexaene

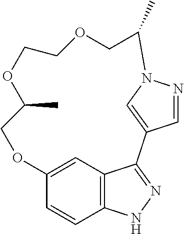 [SMILES: C[C@H]1COc2ccc3[nH]nc(c3c2)-c2cnn(c2)[C@@H](C)COCCO1]

To a solution of (6S,12S)-6,12-dimethyl-19-(oxan-2-yl)-8,11,14-trioxa-4,5,19,20-tetraazatetra cyclo[13.5.2.1^{2,5}.0^{18,21}]tricosa-1(20),2(23),3,15(22),16,18(21)-hexaene (136 mg, 0.32 mmol) in methanol (4 mL) and water (0.6 mL) was added p-toluenesulfonic acid monohydrate (303 mg, 1.60 mmol). The reaction mixture was stirred at 65° C. for 2 h. The reaction mixture was cooled to RT and quenched with a saturated aqueous NaHCO₃ solution until pH basic. Ethyl acetate was added and the phases were separated. The aqueous layer was extracted with ethyl acetate and the combined organic layers were washed with brine, dried over anhydrous sodium sulfate, filtered and evaporated under reduced pressure. The residue was purified by silica gel column chromatography using dichloromethane/methanol 100/0 to 90/10 as eluent to afford (6S,12S)-6,12-dimethyl-8,11,14-trioxa-4,5,19,20-tetraazatetracyclo[13.5.2.1^{2,5}.0^{18,21}]tricosa-1(20), 2(23),3,15(22),16,18(21)-hexaene as a foam.

LCMS method F: [M+H]⁺=343.2, t_{R}=2.13 min

LCMS method G: [M+H]⁺=343.2, t_{R}=2.14 min

1H NMR (400 MHz, d6-DMSO) 12.73 (1H, s), 8.45 (1H, s), 7.83-7.79 (2H, m), 7.39-7.36 (1H, m), 7.01 (1H, dd, J=2.2, 8.8 Hz), 4.65-4.58 (1H, m), 4.30 (1H, dd, J=4.4, 13.5 Hz), 4.19 (1H, dd, J=1.4, 13.2 Hz), 3.85-3.53 (7H, m), 1.51 (3H, d, J=6.8 Hz), 1.20 (3H, d, J=6.3 Hz) ppm.

### Example 90: (12R)-12-methyl-9,13-dioxa-4,5,18,19,22-pentaazatetracyclo[12.5.2.1^{2,5}.0^{17,20}]docosa-1(19),2(22),3,14(21),15,17(20)-hexaene

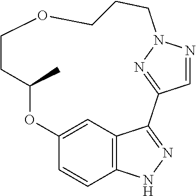 [SMILES: C[C@@H]1CCOCCCn2ncc(n2)-c2n[nH]c3ccc(cc23)O1]

Example 90 is prepared according to the synthesis route described in general Scheme D.

### Preparation of Intermediate 519: 2-[3-[(3S)-3-benzyloxybutoxy]propyl]-4-bromo-triazole

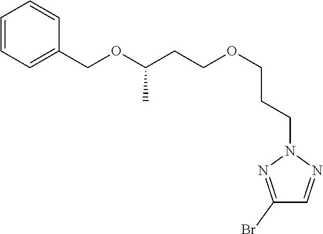 [SMILES: C[C@@H](CCOCCCn1ncc(Br)n1)OCc1ccccc1]

[(3S)-3-benzyloxybutyl] 4-methylbenzenesulfonate was prepared according to the same procedures as [(3R)-3-benzyloxybutyl] 4-methylbenzenesulfonate (intermediate 129) starting from (3S)-butane-1,3-diol.

To a solution of 3-(4-bromotriazol-2-yl)propan-1-ol (intermediate 434) (1.17 g, 5.70 mmol) in anhydrous DMF (30 mL) at 0° C. was added sodium hydride (60% dispersion in mineral oil) (457 mg, 11.418 mmol). The reaction mixture was stirred at 0° C. for 20 min and a solution of [(3S)-3-benzyloxybutyl] 4-methylbenzenesulfonate (2.88 g, 8.62 mmol) in dry DMF (20 mL) was added dropwise. The reaction mixture was stirred at 65° C. The reaction mixture was quenched by addition of water and the mixture was concentrated under reduced pressure. The residue was diluted with brine and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 90/10 as eluent to afford 2-[3-[(3S)-3-benzyloxybutoxy]propyl]-4-bromo-triazole as a colorless oil.

LCMS method F: [M+H]⁺=368.1-370.1, t_{R}=3.04 min

### Preparation of Intermediate 520: [3-[2-[3-[(3S)-3-benzyloxybutoxy]propyl]triazol-4-yl]-1-tetrahydropyran-2-yl-indazol-5-yl]oxy-tert-butyl-dimethyl-silane

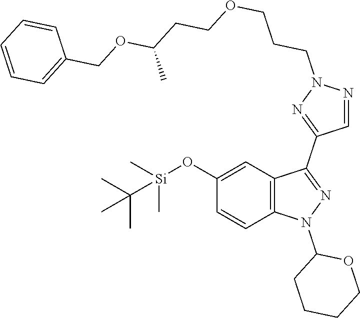 [SMILES: C[C@@H](CCOCCCn1ncc(-c2nn(C3CCCCO3)c3ccc(O[Si](C)(C)C(C)(C)C)cc23)n1)OCc1ccccc1]

To a degassed suspension of 2-[3-[(3S)-3-benzyloxybutoxy]propyl]-4-bromo-triazole (1.99 g, 5.62 mmol), tert-butyl-dimethyl-[1-tetrahydropyran-2-yl-3-(4,4,5,5-tetramethyl-1,3,2-dioxa borolan-2-yl)indazol-5-yl]oxy-silane (intermediate 61) (3.35 g, 7.31 mmol), potassium phosphate tribasic (3.58 g, 16.87 mmol) and XPhos (268 mg, 0.562 mmol) in dioxane (60 mL) and water (6 mL), was added tetrakis(triphenylphosphine)palladium(0) (325 mg, 0.281 mmol). The reaction mixture was stirred at 110° C. for 3 h and at RT for 14 h. The reaction mixture was filtered. Brine was added and the aqueous layer was extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 80/20 as eluent to afford [3-[2-[3-[(3S)-3-benzyloxybutoxy] propyl]triazol-4-yl]-1-tetrahydropyran-2-yl-indazol-5-yl]oxy-tert-butyl-dimethyl-silane as an orange oil.

LCMS method F: [M+H]⁺=620.4, t_{R}=3.97 min

### Preparation of Intermediate 521: (2S)-4-[3-[4-[5-[tert-butyl(dimethyl)silyl]oxy-1-tetrahydropyran-2-yl-indazol-3-yl]triazol-2-yl]propoxy]butan-2-ol

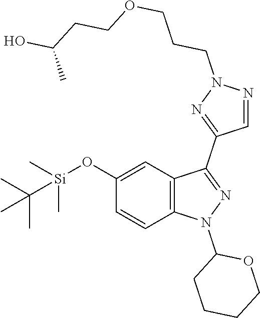 [SMILES: C[C@H](O)CCOCCCn1ncc(-c2nn(C3CCCCO3)c3ccc(O[Si](C)(C)C(C)(C)C)cc23)n1]

To a solution of [3-[2-[3-[(3S)-3-benzyloxybutoxy]propyl]triazol-4-yl]-1-tetrahydropyran-2-yl-indazol-5-yl]oxy-tert-butyl-dimethyl-silane (2.54 g, 4.11 mmol) in ethyl acetate (94 mL) at RT was added palladium 10% on carbon (255 mg). The reaction mixture was stirred under hydrogen atmosphere at RT for 16 h and at 50° C. for 5 h. The reaction mixture was filtered and washed with ethyl acetate. The solvent was removed under reduced pressure. The residue was purified by silica gel column chromatography using cyclohexane/ethyl acetate 100/0 to 60/40 as eluent to afford (2S)-4-[3-[4-[5-[tert-butyl(dimethyl)silyl]oxy-1-tetrahydropyran-2-yl-indazol-3-yl]triazol-2-yl]propoxy]butan-2-ol as a colorless oil.

LCMS method F: [M+H]⁺=530.3, t_{R}=3.49 min

### Preparation of Intermediate 522: [(1S)-3-[3-[4-[5-[tert-butyl(dimethyl)silyl]oxy-1-tetrahydropyran-2-yl-indazol-3-yl]triazol-2-yl]propoxy]-1-methyl-propyl]methane sulfonate

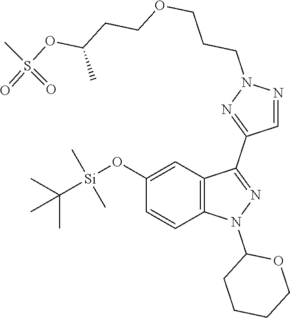 [SMILES: C[C@@H](CCOCCCn1ncc(-c2nn(C3CCCCO3)c3ccc(O[Si](C)(C)C(C)(C)C)cc23)n1)OS(C)(=O)=O]

To a solution of (2S)-4-[3-[4-[5-[tert-butyl(dimethyl)silyl]oxy-1-tetrahydropyran-2-yl-indazol-3-yl]triazol-2-yl]propoxy]butan-2-ol (1.92 g, 3.63 mmol) and triethylamine (1.01 mL, 7.27 mmol) in dry dichloromethane (84 mL) at 0° C. was added methanesulfonyl chloride (422 μL, 5.453 mmol). The reaction mixture was stirred at RT for 3 h. The reaction mixture was quenched with brine and extracted with dichloromethane. The combined organic layers were dried over anhydrous sodium sulfate, filtered and evaporated under reduced pressure to afford [(1S)-3-[3-[4-[5-[tert-butyl(dimethyl)silyl]oxy-1-tetrahydropyran-2-yl-indazol-3-yl]triazol-2-yl]propoxy]-1-methyl-propyl] methanesulfonate as a pale yellow oil which was used in the next step without further purification.

LCMS method F: [M+H]⁺=608.3, t_{R}=3.58 min

### Preparation of Intermediate 523: (12R)-12-methyl-18-(oxan-2-yl)-9,13-dioxa-4,5,18,19,22-pentaazatetracyclo[12.5.2.1^{2,5}.0^{17,20}]docosa-1(19),2(22),3,14(21),15, 17(20)-hexaene

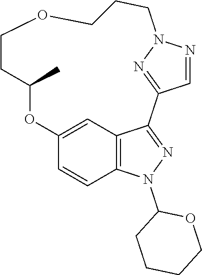 [SMILES: C[C@@H]1CCOCCCn2ncc(n2)-c2nn(C3CCCCO3)c3ccc(cc23)O1]

To a suspension of cesium carbonate (4.4 g, 13.51 mmol) in dry DMF (1.4 L) at 85° C. was added dropwise [(1S)-3-[3-[4-[5-[tert-butyl(dimethyl)silyl]oxy-1-tetrahydropyran-2-yl-indazol-3-yl]triazol-2-yl]propoxy]-1-methyl-propyl] methanesulfonate (2.004 g, 3.379 mmol) in dry DMF (1 L). The reaction mixture was stirred at 85° C. for 3 h. The reaction mixture was concentrated under reduced pressure. The residue was diluted with brine and ethyl acetate. The aqueous layer was extracted with ethyl acetate. The combined organic layers were dried over anhydrous sodium sulfate, filtered and evaporated under reduced pressure. The residue was purified by silica gel column chromatography using cyclohexane/(diisopropyl ether/ethyl acetate (95/5)) 100/0 to 0/100 as eluent to afford (12R)-12-methyl-18-(oxan-2-yl)-9,13-dioxa-4,5,18,19,22-pentaazatetracyclo[12.5.2.1^{2,5}.0^{17,20}]docosa-1(19),2(22),3,14(21),15,17(20)-hexaene as a colorless oil.

LCMS method F: [M+H]⁺=398.2, t_{R}=2.90 min

### Preparation of Example 90: (12R)-12-methyl-9,13-dioxa-4,5,18,19,22-pentaazatetra cyclo[12.5.2.1^{2,5}.0^{17,20}]docosa-1(19),2(22),3,14(21),15,17(20)-hexaene

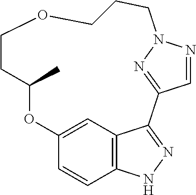 [SMILES: C[C@@H]1CCOCCCn2ncc(n2)-c2n[nH]c3ccc(cc23)O1]

To a solution of afford (12R)-12-methyl-18-(oxan-2-yl)-9,13-dioxa-4,5,18,19,22-pentaazatetra cyclo[12.5.2.1^{2,5}.0^{17,20}]docosa-1(19),2(22),3,14(21),15,17(20)-hexaene (81 mg, 0.204 mmol) in methanol (10 mL) and water (1.3 mL) was added p-toluenesulfonic acid monohydrate (194 mg, 1.02 mmol) at RT. The reaction mixture was stirred at 65° C. for 16 h. The reaction mixture was concentrated under reduced pressure and the residue was neutralized by addition of saturated aqueous NaHCO₃ solution. The mixture was diluted with ethyl acetate and the aqueous layer was extracted with ethyl acetate. The combined organic layers were washed with water and brine, dried over anhydrous sodium sulfate, filtered and evaporated under reduced pressure.

The residue was triturated with diethyl ether, filtered and dried to afford (12R)-12-methyl-9,13-dioxa-4,5,18,19,22-pentaazatetracyclo[12.5.2.1^{2,5}.0^{17,20}]docosa-1(19),2(22),3,14(21),15, 17(20)-hexaene as a solid.

LCMS method F: [M+H]⁺=314.2, t_{R}=2.24 min

LCMS method G: [M+H]⁺=314.3, t_{R}=2.25 min

¹H NMR (400 MHz, DMSO) 13.04 (1H, s), 8.10 (1H, s), 8.09-8.08 (1H, m), 7.46-7.42 (1H, d, J=9.0 Hz), 6.99-6.96 (1H, dd, J=2.5, 9.0 Hz), 4.72-4.59 (2H, m), 4.48-4.40 (1H, m), 4.12-4.06 (1H, m), 3.78-3.71 (1H, m), 3.60-3.49 (2H, m), 2.48-2.43 (1H, m), 2.37-2.24 (2H, m), 1.44-1.37 (4H, m) ppm.

### Example 91: 13-methyl-4-(morpholin-4-yl)-7,10-dioxa-13,19,20-triazatetracyclo [13.5.2.1^{2,6}.0^{18,21}]tricosa-1(20),2,4,6(23),15,17,21-heptaene

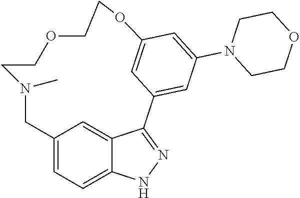 [SMILES: CN1CCOCCOc2cc(cc(N3CCOCC3)c2)-c2n[nH]c3ccc(cc23)C1]

Example 91 is prepared according to the synthesis route described in general Scheme H.

### Preparation of Intermediate 524: tert-butyl N-[2-(2-hydroxyethoxy)ethyl]carbamate

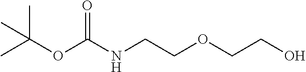

To a solution of 2-(2-aminoethoxy)ethanol (10 mL, 95.11 mmol) in dichloromethane (475 mL) at 0° C. was added Tert-butoxycarbonyl tert-butyl carbonate (24.03 mL, 104.62 mmol). The reaction mixture was stirred at RT for 22 h. The solvent was removed under reduced pressure and the residue was purified by silica gel column chromatography using dichloromethane/methanol 100/0 to 90/10 as eluent to afford tert-butyl N-[2-(2-hydroxyethoxy)ethyl]carbamate a colorless oil.

LCMS method B: [M−Boc]⁺=106.1, t_{R}=0.407 min

### Preparation of Intermediate 525: 2-[2-(tert-butoxycarbonylamino)ethoxy]ethyl 4-methylbenzenesulfonate

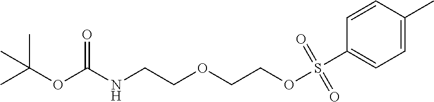 [SMILES: Cc1ccc(S(=O)(=O)OCCOCCNC(=O)OC(C)(C)C)cc1]

To a solution of tert-butyl N-[2-(2-hydroxyethoxy)ethyl]carbamate (12.7 g, 61.87 mmol) in dichloromethane (310 mL) at 0° C. was added triethylamine (12.9 mL, 92.8 mmol) and p-toluenesulfonyl chloride (14.15 g, 74.24 mmol). The mixture was stirred at RT for 20 h. Water was added to the reaction mixture and the aqueous layer was extracted with dichloromethane. The combined organic layers were dried over anhydrous magnesium sulfate, filtered and evaporated under reduced pressure. The residue was purified by silica gel column chromatography using heptane/ethyl acetate 100/0 to 40/60 as eluent to afford 2-[2-(tert-butoxycarbonylamino)ethoxy]ethyl 4-methylbenzenesulfonate as a pale yellow oil.

LCMS method B: [M−Boc]⁺=260.1, t_{R}=0.828 min

### Preparation of Intermediate 526: 3-chloro-5-morpholino-phenol

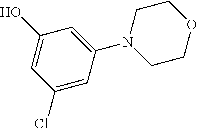 [SMILES: Oc1cc(Cl)cc(N2CCOCC2)c1]

To a mixture of 3-bromo-5-chlorophenol (10 g, 48.20 mmol), morpholine (4.43 mL, 50.61 mmol) and cesium carbonate (62.8 g, 192.8 mmol) in DMF (192 mL) under nitrogen atmosphere was added Pd₂(dba)₃ (4.41 g, 4.82 mmol) and XPhos (4.6 g, 9.64 mmol). The reaction mixture was stirred at 100° C. for 20 h. The reaction mixture was diluted with ethyl acetate, water was added and the aqueous layer was extracted with ethyl acetate. The combined organic layers were washed with brine, dried over anhydrous magnesium sulfate, filtered and evaporated under reduced pressure. The residue was purified by silica gel column chromatography using heptanet/ethyl acetate 100:0 to 65:35 as eluent to afford 3-chloro-5-morpholino-phenol as a brown solid.

LCMS method B: [M+H]⁺=214.1-216.1, t_{R}=0.528 min

### Preparation of Intermediate 527: 3-morpholino-5-(4,4,5,5-tetramethyl-1,3,2-dioxa borolan-2-yl)phenol

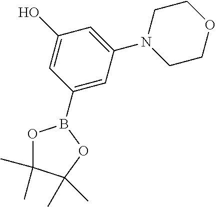 [SMILES: CC1(C)OB(c2cc(O)cc(N3CCOCC3)c2)OC1(C)C]

A mixture of 3-chloro-5-morpholinophenol 1.408 g 6.59 mmol), bis(pinacolato)diboron (1.84 g, 7.24 mmol), tricyclohexylphosphane (185 mg, 0.659 mmol), Pd₂(dba)₃ (181 mg, 0.198 mmol) and potassium acetate (970 mg, 7.02 mmol) in DME (10 mL) under nitrogen atmosphere. The reaction mixture was stirred at 150° C. under microwave irradiations for 1.5 h. The reaction mixture was diluted with ethyl acetate, filtered over a pad of Celite and the solvent was removed under reduced pressure. The residue was purified by silica gel column chromatography using heptane/ethyl acetate 100:0 to 60:40 as eluent to afford 3-morpholino-5-(4,4,5,5-tetramethyl-1,3,2-dioxaborolan-2-yl)phenol as an orange oil.

LCMS method B: [M+H]⁺=306.2, t_{R}=0.689 min

### Preparation of Intermediate 528: ethyl 1H-indazole-5-carboxylate

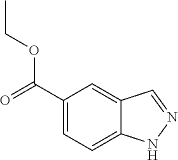

To a solution of 1H-indazole-5-carboxylic acid (15 g, 92.51 mmol) in ethanol (278 mL) was added sulfuric acid (4.93 mL, 92.51 mmol) and the reaction mixture was stirred at 70° C. for 16 h. The reaction mixture was concentrated under reduced pressure and the residue was diluted with ethyl acetate. The resulting precipitate was triturated with ethyl acetate, filtered and dried under reduced pressure to afford ethyl 1H-indazole-5-carboxylate as a pale yellow solid which was used without further purification in the next step.

LCMS method B: [M+H]⁺=191.1, t_{R}=0.532 min

### Preparation of Intermediate 529: ethyl 3-iodo-1H-indazole-5-carboxylate

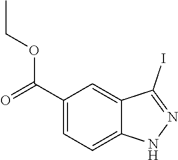

N-Iodosuccinimide (21.86 g, 97.14 mmol) was added to a solution of ethyl 1H-indazole-5-carboxylate (15 g, 92.51 mmol) in DMF (278 mL). The reaction mixture was stirred at RT for 3 h then quenched with a saturated aqueous NaHCO₃ solution and extracted with dichloromethane. The organic layer was washed with brine, filtered and evaporated under reduced pressure. The residue was triturated with heptane, filtered and dried to afford ethyl 3-iodo-1H-indazole-5-carboxylate as a pale brown solid which was used without further purification in the next step.

LCMS method B: [M+H]⁺=317.0, t_{R}=0.743 min

### Preparation of Intermediate 530: ethyl 3-iodo-1-tetrahydropyran-2-yl-indazole-5-carboxylate

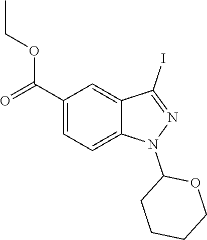 [SMILES: CCOC(=O)c1ccc2c(c1)c(I)nn2C1CCCCO1]

To a solution of 3-iodo-1H-indazole-5-carboxylate (18.1 g, 57.26 mmol) in dichloromethane (172 mL) was added ethyl 4-methylbenzenesulfonic acid hydrate (1.09 g, 5.73 mmol) and 3,4-dihydro-2H-pyran (15.71 mL, 171.78 mmol). The reaction mixture was stirred at RT for 2 h. The reaction mixture was quenched with a saturated aqueous NaHCO₃ solution and the aqueous layer was extracted with dichloromethane. The combined organic layers were dried over anhydrous magnesium sulfate, filtered and evaporated under reduced pressure. The residue was purified by silica gel column chromatography using heptane/ethyl acetate 100/0 to 90/10 as eluent to afford ethyl 3-iodo-1-tetrahydropyran-2-yl-indazole-5-carboxylate a pale yellow solid.

LCMS method B: [M+H]⁺=401.0, t_{R}=1.07 min

### Preparation of Intermediate 531: ethyl 3-(3-hydroxy-5-morpholinophenyl)-1-(tetrahydro-2H-pyran-2-yl)-1H-indazole-5-carboxylate

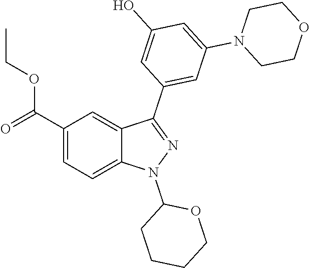 [SMILES: CCOC(=O)c1ccc2c(c1)c(-c1cc(O)cc(N3CCOCC3)c1)nn2C1CCCCO1]

To a mixture of ethyl 3-iodo-1-tetrahydropyran-2-yl-indazole-5-carboxylate (2 g, 4.997 mmol), 3-morpholino-5-(4,4,5,5-tetramethyl-1,3,2-dioxaborolan-2-yl)phenol (intermediate 527) (1.98 g, 6.49 mmol) and triethylamine (2.050 ml, 14.991 mmol) in dioxane (37.5 mL) and water (12.5 mL) under nitrogen atmosphere was added tetrakis(triphenylphosphine)palladium(0) (289 mg, 0.250 mmol) and XPhos (238 mg, 0.500 mmol). The reaction mixture was stirred at 110° C. for 16 h. The reaction mixture was diluted with ethyl acetate, washed with water and extracted with ethyl acetate. The combined organic layers were dried over anhydrous magnesium sulfate, filtered and evaporated under reduced pressure. The residue was purified by silica gel column chromatography using heptane/ethyl acetate 100/0 to 50/50 as eluent to afford ethyl 3-(3-hydroxy-5-morpholinophenyl)-1-(tetrahydro-2H-pyran-2-yl)-1H-indazole-5-carboxylate as a pale white solid.

LCMS method B: [M+H]⁺=452.2, t_{R}=0.957 min

### Preparation of Intermediate 532: ethyl 3-[3-[2-[2-(tert-butoxycarbonylamino)ethoxy] ethoxy]-5-morpholino-phenyl]-1-tetrahydropyran-2-yl-indazole-5-carboxylate

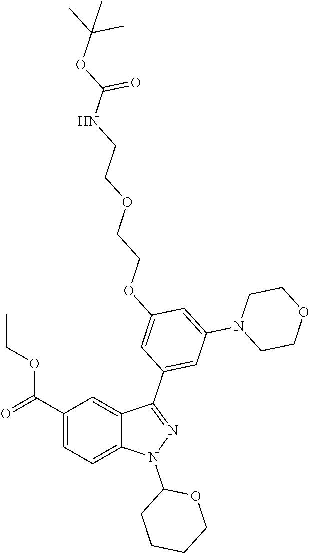 [SMILES: CCOC(=O)c1ccc2c(c1)c(-c1cc(OCCOCCNC(=O)OC(C)(C)C)cc(N3CCOCC3)c1)nn2C1CCCCO1]

A mixture of ethyl 3-(3-hydroxy-5-morpholinophenyl)-1-(tetrahydro-2H-pyran-2-yl)-1H-indazole-5-carboxylate (2 g, 4.42 mmol), 2-(2-((tert-butoxycarbonyl)amino)ethoxy)ethyl 4-methylbenzenesulfonate (intermediate 525) (2.38 g, 6.64 mmol) and cesium carbonate (2.88 g, 8.85 mmol) in DMF (33 mL) was stirred at 80° C. for 16 h. The reaction mixture was diluted with ethyl acetate and washed with water. The organic layer was dried over anhydrous magnesium sulfate, filtered and evaporated under reduced pressure. The residue was purified by silica gel column chromatography using heptane/ethyl acetate 100/0 to 0/100 as eluent to afford ethyl 3-[3-[2-[2-(tert-butoxycarbonylamino)ethoxy]ethoxy]-5-morpholino-phenyl]-1-tetrahydropyran-2-yl-indazole-5-carboxylate as a yellowish oil.

LCMS method B: [M+H]⁺=639.3, t_{R}=1.213 min

### Preparation of Intermediate 533: 3-[3-[2-[2-(tert-butoxycarbonylamino)ethoxy] ethoxy]-5-morpholino-phenyl]-1-tetrahydropyran-2-yl-indazole-5-carboxylic acid

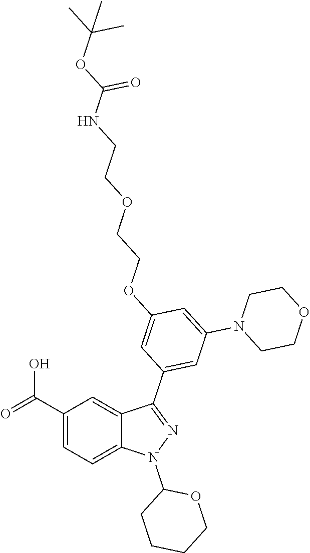 [SMILES: CC(C)(C)OC(=O)NCCOCCOc1cc(-c2nn(C3CCCCO3)c3ccc(C(=O)O)cc23)cc(N2CCOCC2)c1]

To a solution of 3-[3-[2-[2-(tert-butoxycarbonylamino)ethoxy]ethoxy]-5-morpholino-phenyl]-1-tetrahydropyran-2-yl-indazole-5-carboxylate (950 mg, 1.48 mmol) in THF (4 mL) and water (1 mL) was added lithium hydroxide monohydrate (305 mg, 7.43 mmol). The reaction mixture was stirred at 60° C. for 18 h. The reaction mixture was acidified with HCl 1N to pH 6 and extracted with ethyl acetate. The organic layer was dried over anhydrous magnesium sulfate, filtered and evaporated under reduced pressure. The residue was purified by silica gel column chromatography using dichloromethane/methanol 100/0 to 90/10 as eluent to afford 3-[3-[2-[2-(tert-butoxycarbonylamino)ethoxy]ethoxy]-5-morpholino-phenyl]-1-tetrahydropyran-2-yl-indazole-5-carboxylic acid as a yellow foam.

LCMS method B: [M+H]⁺=611.3, t_{R}=1.944 min

### Preparation of Intermediate 534: 3-[3-[2-(2-aminoethoxy)ethoxy]-5-morpholino-phenyl]-1H-indazole-5-carboxylic acid hydrochloride

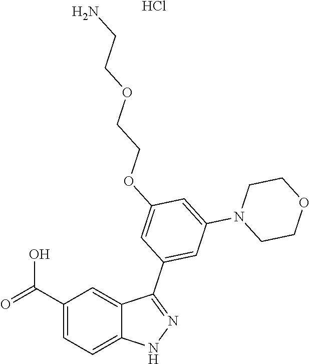 [SMILES: Cl ; NCCOCCOc1cc(-c2n[nH]c3ccc(C(=O)O)cc23)cc(N2CCOCC2)c1]

3-[3-[2-[2-(tert-butoxycarbonylamino)ethoxy]ethoxy]-5-morpholino-phenyl]-1-tetrahydro pyran-2-yl-indazole-5-carboxylic acid (780 mg, 1.277 mmol) in hydrogen chloride solution (4M in dioxane) (12 ml) was stirred at RT for 16 h. The reaction mixture was concentrated under reduced pressure and co-evaporated with heptane to afford 3-[3-[2-(2-aminoethoxy)ethoxy]-5-morpholino-phenyl]-1H-indazole-5-carboxylic acid hydrochloride as a cream solid which was used without further purification.

LCMS method B: [M+H]⁺=427.2, t_{R}=0.392 min

### Preparation of Intermediate 535: 4-(morpholin-4-yl)-7,10-dioxa-13,19,20-triazatetra cyclo[13.5.2.1^{2,6}.0^{18,21}]tricosa-1(20),2,4,6(23),15,17,21-heptaen-14-one

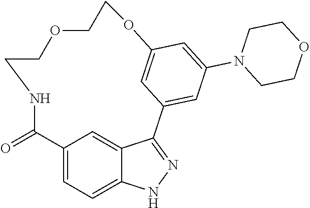 [SMILES: O=C1NCCOCCOc2cc(cc(N3CCOCC3)c2)-c2n[nH]c3ccc1cc23]

To a solution of HBTU (2.39 g, 6.31 mmol) in DMA (440 mL) was added DIPEA (7.5 mL, 42.12 mmol) then 3-[3-[2-(2-aminoethoxy)ethoxy]-5-morpholino-phenyl]-1H-indazole-5-carboxylic acid hydrochloride (975 mg, 2.106 mmol) in DMA (190 mL) was added dropwise. The reaction mixture was stirred at RT for 10 min. Ammonia 30% in water (6 mL) was added and the mixture was stirred at RT for 30 min. The solvent was evaporated under reduced pressure. The residue was dissolved in ethyl acetate and washed with a saturated aqueous NaHCO₃ solution. The aqueous layer was extracted with ethyl acetate. The combined organic layers were dried over anhydrous magnesium sulfate, filtered and evaporated under reduced pressure. The residue was purified by silica gel column chromatography using dichloromethane/methanol 100/0 to 97/3 as eluent to afford 4-(morpholin-4-yl)-7,10-dioxa-13,19,20-triazatetracyclo[13.5.2.1^{2,6}.0^{18,21}]tricosa-1(20),2,4,6(23),15,17,21-heptaen-14-one as a cream solid.

LCMS method B: [M+H]⁺=409.2, t_{R}=0.592 min

### Preparation of Intermediate 536: tert-butyl 4-(morpholin-4-yl)-14-oxo-7,10-dioxa-13,19,20-triazatetracyclo[13.5.2.1^{2,6}.0^{18,21}]tricosa-1(20),2,4,6(23),15,17,21-heptaene-19-carboxylate

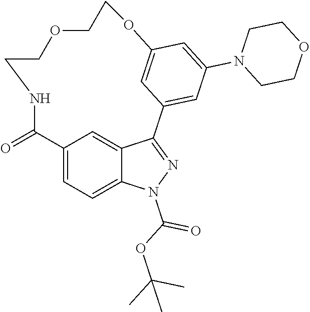 [SMILES: CC(C)(C)OC(=O)n1nc2c3cc(ccc31)C(=O)NCCOCCOc1cc-2cc(N2CCOCC2)c1]

To a solution of 4-(morpholin-4-yl)-7,10-dioxa-13,19,20-triazatetracyclo[13.5.2.1^{2,6}.0^{18,21}] tricosa-1(20),2,4,6(23),15,17,21-heptaen-14-one (180 mg, 0.441 mmol) in dichloromethane (1 mL) and DMA (1 mL) was added 4-(dimethylamino)pyridine (11 mg, 0.088 mmol) and di-tert-butyl dicarbonate (106 mg, 0.485 mmol). The reaction mixture was stirred at RT for 2 h. The reaction mixture was concentrated under reduced pressure and the residue was purified by silica gel column chromatography using dichloromethane/methanol 100/0 to 99/1 as eluent to afford tert-butyl-4-(morpholin-4-yl)-14-oxo-7,10-dioxa-13,19,20-triazatetracyclo[13.5.2.1^{2,6}.0^{18,21}] tricosa-1(20),2,4,6(23),15,17,21-heptaene-19-carboxylate as a white solid.

LCMS method B: [M+H]⁺=509.2, t_{R}=0.984 min

### Preparation of Intermediate 537: tert-butyl13-methyl-4-(morpholin-4-yl)-14-oxo-7,10-dioxa-13,19,20-triazatetracyclo[13.5.2.1², 0^{18,21}]tricosa-1(20),2,4,6(23),15,17, 21-heptaene-19-carboxylate

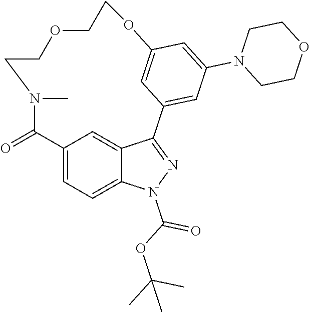 [SMILES: CN1CCOCCOc2cc(cc(N3CCOCC3)c2)-c2nn(C(=O)OC(C)(C)C)c3ccc(cc23)C1=O]

To a solution of tert-butyl 4-(morpholin-4-yl)-14-oxo-7,10-dioxa-13,19,20-triazatetracyclo [13.5.2.1^{2,6}.0^{18,21}]tricosa-1(20),2,4,6(23),15,17,21-heptaene-19-carboxylate (272 mg, 0.535 mmol) in dry DMF (13 mL) at 0° C. was added sodium hydride (60% dispersion in mineral oil) (26 mg, 0.669 mmol) under nitrogen atmosphere, The reaction mixture was stirred at RT for 30 min and iodomethane (37 □L, 0.589 mmol) was added. The reaction mixture was stirred at RT for 30 min. Water was added and the aqueous layer was extracted with ethyl acetate. The combined organic layers were dried over anhydrous magnesium sulfate, filtered and evaporated under reduced pressure. The residue was purified by silica gel chromatography using dichloromethane/methanol 100/0 to 95/5 as eluent to afford tert-butyl13-methyl-4-(morpholin-4-yl)-14-oxo-7,10-dioxa-13,19,20-triazatetracyclo[13.5.2.1^{2,6}.0^{18,21}]tricosa-1(20),2,4,6(23), 15,17,21-heptaene-19-carboxylate as a beige solid.

LCMS method C: [M+H]⁺=523.4, t_{R}=4.01 min

### Preparation of Example 91: 13-methyl-4-(morpholin-4-yl)-7,10-dioxa-13,19,20-triazatetracyclo[13.5.2.1^{2,6}.0^{18,21}]tricosa-1(20),2,4,6(23),15,17,21-heptaene

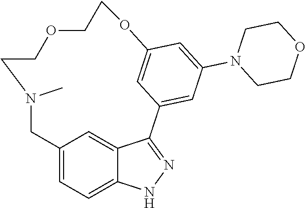 [SMILES: CN1CCOCCOc2cc(cc(N3CCOCC3)c2)-c2n[nH]c3ccc(cc23)C1]

To a stirred mixture of tert-butyl13-methyl-4-(morpholin-4-yl)-14-oxo-7,10-dioxa-13,19,20-triazatetracyclo[13.5.2.1^{2,6}.0^{18,21}]tricosa-1(20),2,4,6(23),15,17,21-heptaene-19-carboxylate (11 mg, 0.026 mmol) in dioxane (0.5 mL) under nitrogen atmosphere was added a solution of titanium tetrachloride (1M in dichloromethane) (26 μl, 0.026 mmol). The mixture was stirred at RT for 5 min then lithium aluminum hydride (3 mg, 0.078 mmol) was added. The reaction mixture was stirred at RT for 3 h. The reaction mixture was cooled to 0° C. and quenched with a 1M NaOH solution. The mixture was diluted with ethyl acetate and the aqueous layer was extracted with ethyl acetate. The combined organic layers were dried over anhydrous magnesium sulfate, filtered and evaporated under reduced pressure. The residue was purified by silica gel column chromatography using dichloromethane/methanol 100/0 to 99/1 as eluent to afford 13-methyl-4-(morpholin-4-yl)-7,10-dioxa-13,19,20-triazatetracyclo[13.5.2.1^{2,6}.0^{18,21}] tricosa-1(20),2,4,6(23),15,17,21-heptaene as a foam.

LCMS method E: [M+H]⁺=409.1, t_{R}=1.835 min

LCMS method D: [M+H]⁺=409.1, t_{R}=3.871 min

1H NMR (300 MHz, d6-DMSO) 13.00 (s, J=3.7 Hz, 1H), 8.47 (s, 1H), 7.65 (s, 1H), 7.47 (d, J=8.6 Hz, 1H), 7.14 (d, J=6.4 Hz, 2H), 6.49 (s, 1H), 4.32 (t, J=3.7 Hz, 2H), 3.81-3.71 (m, J=4.7 Hz, 6H), 3.69 (s, 2H), 3.61 (brt, J=4.0 Hz, 2H), 3.14 (t, J=4.5 Hz, 4H), 2.60 (brs, 2H), 2.42 (m, 3H) ppm.

### Example 92: (12S)-12-methyl-9,13-dioxa-4,5,18,19,22-pentaazatetracyclo[12.5.2.1^{2,5}.0^{17,20}]docosa-1(19),2(22),3,14(21),15,17(20)-hexaene

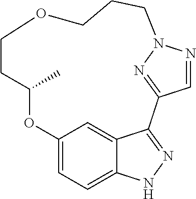 [SMILES: C[C@H]1CCOCCCn2ncc(n2)-c2n[nH]c3ccc(cc23)O1]

Example 92 is prepared according to the synthesis route described in general Scheme D.

### Preparation of Intermediate 538: 2-[3-[(3R)-3-benzyloxybutoxy]propyl]-4-bromo-triazole

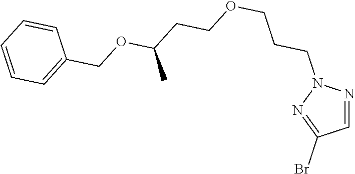 [SMILES: C[C@H](CCOCCCn1ncc(Br)n1)OCc1ccccc1]

To a solution of 3-(4-bromotriazol-2-yl)propan-1-ol (intermediate 434) (951 mg, 4.61 mmol) in anhydrous DMF (25 mL) at 0° C. was added sodium hydride (60% dispersion in mineral oil) (369 mg, 9.234 mmol). The reaction mixture was stirred at 0° C. for 20 min then a solution of [(3R)-3-benzyloxybutyl]-4-methylbenzenesulfonate (intermediate 129) (2.27 g, 6.79 mmol) in dry DMF (15 mL) was added dropwise and the resulting mixture was stirred at 65° C. for 24 h then RT for 72 h. The reaction mixture was quenched by addition of water then concentrated under reduced pressure. The residue was diluted with a saturated aqueous sodium chloride solution and extracted with ethyl acetate. The combined organic layers were dried over anhydrous sodium sulfate, filtered and evaporated under reduced pressure. The residue was purified by silica gel column chromatography eluting with cyclohexane/ethyl acetate 100/0 to 90/10 as eluent to afford 2-[3-[(3R)-3-benzyloxybutoxy]propyl]-4-bromo-triazole as a colorless oil.

LCMS method F: [M+H]⁺=368.1-370.1, t_{R}=3.04 min

### Preparation of Intermediate 539: [3-[2-[3-[(3R)-3-benzyloxybutoxy]propyl]triazol-4-yl]-1-tetrahydropyran-2-yl-indazol-5-yl]oxy-tert-butyl-dimethyl-silane

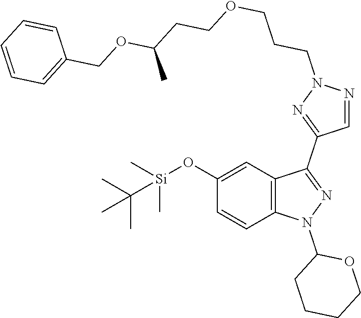 [SMILES: C[C@H](CCOCCCn1ncc(-c2nn(C3CCCCO3)c3ccc(O[Si](C)(C)C(C)(C)C)cc23)n1)OCc1ccccc1]

To a degassed suspension of 2-[3-[(3R)-3-benzyloxybutoxy]propyl]-4-bromo-triazole (1.25 g, 3.39 mmol), tert-butyl-dimethyl-[1-tetrahydropyran-2-yl-3-(4,4,5,5-tetramethyl-1,3,2-dioxa borolan-2-yl) indazol-5-yl]oxy-silane (intermediate 61) (2.02 g, 4.41 mmol), potassium phosphate tribasic (2.16 g, 10.19 mmol), and XPhos (162 mg, 0.34 mmol) in dioxane (36 mL) and water (3.6 mL), was added tetrakis(triphenylphosphine)palladium(0) (197 mg, 0.170 mmol). The reaction mixture was stirred at 110° C. for 3 h then RT for 14 h. The reaction was filtered and the filtrate was diluted with a saturated aqueous sodium chloride solution and extracted with ethyl acetate. The combined organic layer was dried over anhydrous sodium sulfate, filtered and evaporated under reduced pressure. The residue was purified by silica gel column chromatography using cyclohexane/ethyl acetate 100/0 to 80/20 as eluent to afford [3-[2-[3-[(3R)-3-benzyloxybutoxy]propyl]triazol-4-yl]-1-tetrahydropyran-2-yl-indazol-5-yl]oxy-tert-butyl-dimethyl-silane as a yellow oil.

LCMS method J: [M+H]⁺=620.4, t_{R}=6.39 min

### Preparation of Intermediate 540: (2R)-4-[3-[4-[5-[tert-butyl(dimethyl)silyl]oxy-1-tetrahydropyran-2-yl-indazol-3-yl]triazol-2-yl]propoxy]butan-2-ol

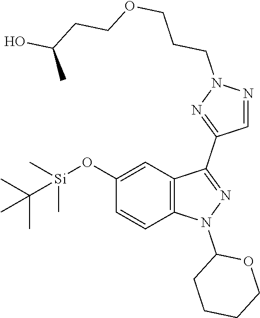 [SMILES: C[C@@H](O)CCOCCCn1ncc(-c2nn(C3CCCCO3)c3ccc(O[Si](C)(C)C(C)(C)C)cc23)n1]

To a solution of [3-[2-[3-[(3R)-3-benzyloxybutoxy]propyl]triazol-4-yl]-1-tetrahydropyran-2-yl-indazol-5-yl]oxy-tert-butyl-dimethyl-silane (1.569 g, 2.535 mmol) in ethyl acetate (70 mL) at RT was added palladium 10% on carbon (157 mg). The reaction mixture was stirred under hydrogen atmosphere at 50° C. for 20 h. The reaction mixture was filtered then washed with ethyl acetate. The reaction mixture was concentrated under reduced pressure. The residue was purified by silica gel column chromatography eluting with cyclohexane/ethyl acetate 100/0 to 60/40 as eluent to give (2R)-4-[3-[4-[5-[tert-butyl(dimethyl)silyl]oxy-1-tetrahydropyran-2-yl-indazol-3-yl]triazol-2-yl]propoxy]butan-2-ol as a very pale yellow oil.

LCMS method F: [M+H]⁺=530.4, t_{R}=3.53 min

### Preparation of Intermediate 541: [(1R)-3-[3-[4-[5-[tert-butyl(dimethyl)silyl]oxy-1-tetrahydropyran-2-yl-indazol-3-yl]triazol-2-yl]propoxy]-1-methyl-propyl]methane sulfonate

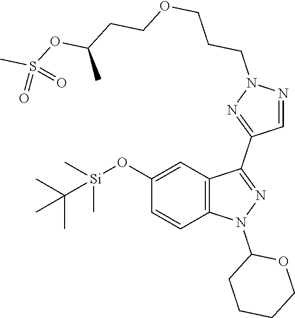 [SMILES: C[C@H](CCOCCCn1ncc(-c2nn(C3CCCCO3)c3ccc(O[Si](C)(C)C(C)(C)C)cc23)n1)OS(C)(=O)=O]

To a solution of (2R)-4-[3-[4-[5-[tert-butyl(dimethyl)silyl]oxy-1-tetrahydropyran-2-yl-indazol-3-yl]triazol-2-yl]propoxy]butan-2-ol (1.179 g, 2.229 mmol) and triethylamine (0.62 mL, 4.458 mmol) in dry dichloromethane (50 mL) at 0° C. was added methanesulfonyl chloride (259 μL, 3.344 mmol). The reaction mixture was stirred at RT for 16 h. The reaction was quenched with a saturated aqueous sodium chloride solution and extracted with dichloromethane. The combined organic layers were dried over anhydrous sodium sulfate, filtered and evaporated under reduced pressure to afford [(1R)-3-[3-[4-[5-[tert-butyl(dimethyl)silyl]oxy-1-tetrahydropyran-2-yl-indazol-3-yl]triazol-2-yl]propoxy]-1-methyl-propyl]methanesulfonate a pale pink oil which was used in the next step without further purification.

LCMS method F: [M+H]⁺=608.3, t_{R}=3.58 min

### Preparation of Intermediate 542: (12S)-12-methyl-18-(oxan-2-yl)-9,13-dioxa-4,5,18, 19,22-pentaazatetracyclo[12.5.2.1^{2,5}.0^{17,20}]docosa-1(19),2(22),3,14(21),15,17(20)-hexaene

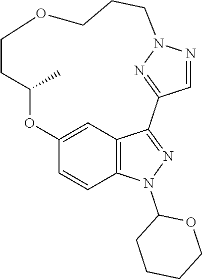 [SMILES: C[C@H]1CCOCCCn2ncc(n2)-c2nn(C3CCCCO3)c3ccc(cc23)O1]

To a suspension of cesium carbonate (2.74 g, 8.41 mmol) in dry DMF (800 mL) at 85° C. was added dropwise [(1R)-3-[3-[4-[5-[tert-butyl(dimethyl)silyl]oxy-1-tetrahydropyran-2-yl-indazol-3-yl]triazol-2-yl] propoxy]-1-methyl-propyl] methanesulfonate (1.27 g, 2.10 mmol) in dry DMF (850 mL). The reaction mixture was heated to 85° C. for 72 h. The reaction mixture was concentrated under reduced pressure then diluted with a saturated aqueous sodium chloride solution and ethyl acetate. The phases were separated and the aqueous layer was extracted with ethyl acetate. The combined organic layers were dried over anhydrous sodium sulfate, filtered and evaporated under reduced pressure. The residue was purified by silica gel column chromatography using cyclohexane/(ethyl acetate/diisopropyl ether (3-1)), 100/0 to 70/30 as eluent to afford (12S)-12-methyl-18-(oxan-2-yl)-9,13-dioxa-4,5,18,19,22-pentaazatetracyclo [12.5.2.1^{2,6}.0^{17,20}]docosa-1(19),2(22),3,14(21),15,17(20)-hexaene as a white sticky solid.

LCMS method F: [M+H]⁺=398.4, t_{R}=2.88 min

### Preparation of Example 92: (12S)-12-methyl-9,13-dioxa-4,5,18,19,22-pentaazatetra cyclo[12.5.2.1^{2,5}.0^{17,20}]docosa-1(19),2(22),3,14(21),15,17(20)-hexaene

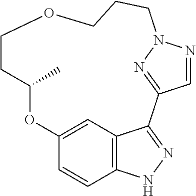 [SMILES: C[C@H]1CCOCCCn2ncc(n2)-c2n[nH]c3ccc(cc23)O1]

To a solution of (12S)-12-methyl-18-(oxan-2-yl)-9,13-dioxa-4,5,18,19,22-pentaazatetracyclo [12.5.2.1^{2,5}.0^{17,20}]docosa-1(19),2(22),3,14(21),15,17(20)-hexaene (216 mg, 0.544 mmol) in methanol (27 mL) and water (3.5 mL) at RT was added p-toluenesulfonic acid monohydrate (517 mg, 2.720 mmol). The reaction mixture was heated to 65° C. for 16 h. The reaction mixture was concentrated under reduced pressure and the residue was neutralized by slow addition of saturated aqueous NaHCO₃ solution then diluted with ethyl acetate. The phases were separated and the aqueous layer was extracted with ethyl acetate. The combined organic layers were washed with water then brine, dried over anhydrous sodium sulfate, filtered and evaporated under reduced pressure. The residue was recrystallized from diisopropylethyl ether to afford (12S)-12-methyl-9,13-dioxa-4,5,18,19,22-pentaazatetracyclo[12.5.2.1^{2,5}.0^{17,20}]docosa-1(19), 2(22),3,14(21),15,17(20)-hexaene as a powder.

LCMS method F: [M+H]⁺=314.3, t_{R}=2.19 min

LCMS method G: [M+H]⁺=314.3, t_{R}=2.18 min

1H NMR (400 MHz, d6-DMSO) 13.04 (1H, s), 8.10 (1H, s), 8.08 (1H, m), 7.47-7.43 (1H, d, J=8.76 Hz), 6.99-9.96 (1H, dd, J=2.5, 8.9 Hz), 4.72-4.59 (2H, m), 4.47-4.40 (1H, m), 4.12-4.06 (1H, m), 3.78-3.71 (1H, m), 3.60-3.49 (2H, m), 2.49-2.43 (1H, m), 2.40-2.23 (2H, m), 1.45-1.36 (4H, m) ppm.

### Example 93: (6S,13R)-6,13-dimethyl-8,11,14-trioxa-4,5,19,20-tetraazatetracyclo[13.5.2.1^{2,5}.0^{18,21}]tricosa-1(20),2(23),3,15(22),16,18(21)-hexaene

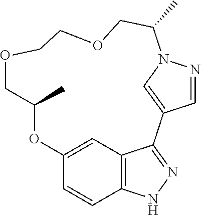 [SMILES: C[C@@H]1COCCOC[C@H](C)n2cc(cn2)-c2n[nH]c3ccc(cc23)O1]

Example 93 is prepared according to the synthesis route described in general Scheme D.

### Preparation of Intermediate 543: 2-[2-[(2S)-2-benzyloxypropoxy]ethoxy]tetrahydro pyran

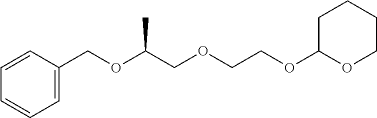 [SMILES: C[C@@H](COCCOC1CCCCO1)OCc1ccccc1]

To a solution of (2S)-2-benzyloxypropan-1-ol (intermediate 104) (3.45 g, 20.78 mmol) in DMF (88 mL) was added portionwise sodium hydride (60% dispersion in mineral oil) (2.08 g, 31.17 mmol) at 0° C. The reaction mixture was stirred at RT for 30 min then 2-(2-bromoethoxy)tetrahydropyran (9.4 mL, 62.35 mmol) was added. The reaction mixture was heated at 55° C. overnight. Additional sodium hydride (60% dispersion in mineral oil) (1.04 g, 15.59 mmol) and 2-(2-bromoethoxy)tetrahydropyran (4.7 mL, 31.18 mmol) were added at RT. The reaction mixture was heated at 55° C. for 5 h. The reaction mixture was quenched with water, then partially concentrated under reduced pressure. Ethyl acetate and water were added, the phases were separated and the aqueous layer was extracted with ethyl acetate. The organic layer was washed with water, 10% aqueous lithium chloride solution and brine, dried over anhydrous sodium sulfate, filtered and evaporated under reduced pressure. The residue was purified by silica gel column chromatography using cyclohexane/ethyl acetate 100/0 to 80/20 as eluent to afford 2-[2-[(2S)-2-benzyloxypropoxy]ethoxy]tetrahydropyran as a pale yellow liquid.

¹H NMR (400 MHz, CDCl₃) δ 7.40-7.27 (m, 5H), 4.69-4.65 (m, 3H), 3.93-3.87 (m, 2H), 3.81-3.73 (m, 1H), 3.71-3.68 (m, 2H), 3.66-3.59 (m, 2H), 3.56-3.49 (m, 2H), 1.90-1.81 (m, 1H), 1.77-1.70 (m, 1H), 1.67-1.51 (m, 4H), 1.22 (dd, J=1.3, 6.4 Hz, 3H) ppm.

### Preparation of Intermediate 544: 2-[(2S)-2-benzyloxypropoxy]ethanol

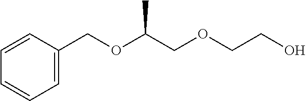

To a solution of 2-[2-[(2S)-2-benzyloxypropoxy]ethoxy]tetrahydropyran (1.13 g, 3.84 mmol) in methanol (80 mL) was added p-toluenesulfonic acid monohydrate (73 mg, 0.38 mmol). The reaction mixture was stirred at RT for 1 h. Methanol was partially evaporated under reduced pressure and the reaction was quenched with a saturated aqueous NaHCO₃ solution. Ethyl acetate was added, the layers were separated and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent to afford 2-[(2S)-2-benzyloxypropoxy]ethanol as colorless oil.

¹H NMR (400 MHz, CDCl₃) 7.38-7.30 (m, 5H), 4.67 (d, J=11.7 Hz, 1H), 4.60 (d, J=11.7 Hz, 1H), 3.79-3.73 (m, 3H), 3.64-3.51 (m, 4H), 2.00 (s, 1H), 1.23 (d, J=6.7 Hz, 3H) ppm.

### Preparation of Intermediate 545: 2-[(2S)-2-benzyloxypropoxy]ethyl methane sulfonate

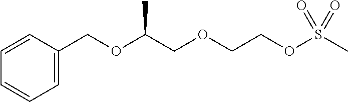 [SMILES: C[C@@H](COCCOS(C)(=O)=O)OCc1ccccc1]

To a solution of 2-[(2S)-2-benzyloxypropoxy]ethanol (350 mg, 1.67 mmol) in dichlormethane (8 mL) at 0° C. was added triethylamine (0.46 mL, 3.34 mmol) and methanesulfonyl chloride (0.16 mL, 2.13 mmol). The reaction mixture was stirred at RT for 1.5. Water and dichloro methane were added and the layers were separated. The organic layer was washed with a saturated aqueous NaHCO₃ then a saturated ammonium chloride solution. The organic layer was dried over anhydrous sodium sulfate, filtered and evaporated under reduced pressure to afford 2-[(2S)-2-benzyloxypropoxy]ethylmethanesulfonate as a yellow oil which was used in the next step without further purification.

LCMS method F: [M+H]⁺=289.1, t_{R}=2.36 min

### Preparation of Intermediate 546: 1-[(1S)-2-[2-[(2S)-2-benzyloxypropoxy]ethoxy]-1-methyl-ethyl]-4-bromo-pyrazole

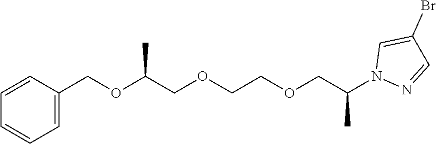 [SMILES: C[C@@H](COCCOC[C@H](C)n1cc(Br)cn1)OCc1ccccc1]

To a solution of 2-[(2S)-2-benzyloxypropoxy]ethylmethanesulfonate (250 mg, 1.22 mmol) in dry DMF (3 mL) was added sodium hydride (60% dispersion in mineral oil) (73 mg, 1.83 mmol). The reaction mixture was stirred at RT for 10 min and a solution of 2-[(2S)-2-(benzyloxy)propoxy]ethyl methanesulfonate (457 mg, 1.59 mmol) in dry DMF (2 mL) was added. The reaction mixture was stirred at RT for 4 h. Additional sodium hydride (60% dispersion in mineral oil) (73 mg, 1.83 mmol) was added and the resulting mixture was stirred at RT overnight. The reaction was quenched with water and ethyl acetate was added. The layers were separated and the aqueous layer was extracted with ethyl acetate. The combined organic layers were dried over anhydrous sodium sulfate, filtered and evaporated under reduced pressure. The residue was purified on silica gel column chromatography using cyclohexane/ethyl acetate 100/0 to 70/30 as eluent. to afford 1-[(1S)-2-[2-[(2S)-2-benzyloxy propoxy]ethoxy]-1-methyl-ethyl]-4-bromo-pyrazole as a colorless oil.

LCMS method F: [M+H]⁺=397.3-399.3, t_{R}=2.87 min

### Preparation of Intermediate 547: [3-[1-[(1S)-2-[2-[(2S)-2-benzyloxypropoxy]ethoxy]-1-methyl-ethyl]pyrazol-4-yl]-1-tetrahydropyran-2-yl-indazol-5-yl]oxy-tert-butyl-dimethyl-silane

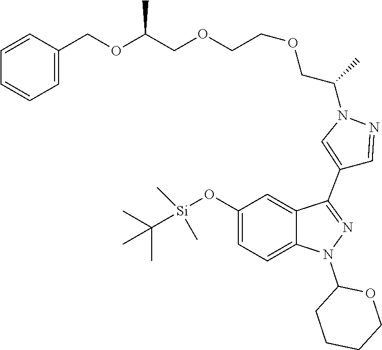 [SMILES: C[C@@H](COCCOC[C@H](C)n1cc(-c2nn(C3CCCCO3)c3ccc(O[Si](C)(C)C(C)(C)C)cc23)cn1)OCc1ccccc1]

To a suspension of 1-[(1S)-2-[2-[(2S)-2-benzyloxypropoxy]ethoxy]-1-methyl-ethyl]-4-bromo-pyrazole (152 mg, 0.38 mmol) in dioxane (2.3 mL) and water (0.2 mL) was added tert-butyl-dimethyl-[1-tetrahydropyran-2-yl-3-(4,4,5,5-tetramethyl-1,3,2-dioxaborolan-2-yl)indazol-5-yl]oxy-silane (228 mg, 0.50 mmol) and potassium phosphate tribasic (242 mg, 1.14 mmol). The reaction was degassed by purged with argon for 10 min then tetrakis(triphenylphosphine) palladium(0) (22 mg, 0.02 mmol) and Xphos (18 mg, 0.04 mmol) were added. The resulting mixture was stirred at 110° C. for 12 h. The mixture was cooled to RT and the reaction mixture was diluted with water and ethyl acetate. The layers were separated and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 60/40 as eluent to afford [3-[1-[(1S)-2-[2-[(2S)-2-benzyloxypropoxy]ethoxy]-1-methyl-ethyl] pyrazol-4-yl]-1-tetrahydropyran-2-yl-indazol-5-yl]oxy-tert-butyl-dimethyl-silane as a yellow oil.

LCMS method F: [M+H]⁺=649.4, t_{R}=3.77 min

### Preparation of Intermediate 548: (2S)-1-[2-[(2S)-2-[4-[5-[tert-butyl(dimethyl) silyl]oxy-1-tetrahydropyran-2-yl-indazol-3-yl]pyrazol-1-yl]propoxy]ethoxy]propan-2-ol

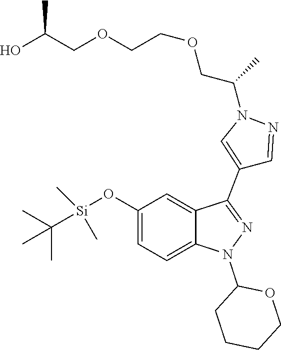 [SMILES: C[C@H](O)COCCOC[C@H](C)n1cc(-c2nn(C3CCCCO3)c3ccc(O[Si](C)(C)C(C)(C)C)cc23)cn1]

To a suspension of [3-[1-[(1S)-2-[2-[(2S)-2-benzyloxypropoxy]ethoxy]-1-methyl-ethyl] pyrazol-4-yl]-1-tetrahydropyran-2-yl-indazol-5-yl]oxy-tert-butyl-dimethyl-silane (143 mg, 0.22 mmol) in ethanol (2 mL) under argon was added palladium on charcoal 10% (2 mg). The reaction mixture was stirred under hydrogen atmosphere for 3 h. The mixture was filtered on a pad of Celite and washed with ethanol and ethyl acetate. The filtrate was evaporated under reduced pressure to afford (2S)-1-[2-[(2S)-2-[4-[5-[tert-butyl(dimethyl)silyl]oxy-1-tetrahydro pyran-2-yl-indazol-3-yl]pyrazol-1-yl]propoxy]ethoxy]propan-2-ol as a pale brown oil. This product was used in the next step without further purification.

LCMS method F: [M+H]⁺=559.5, t_{R}=3.31 min

### Preparation of Intermediate 549: [(1S)-2-[2-[(2S)-2-[4-[5-[tert-butyl(dimethyl)silyl] oxy-1-tetrahydropyran-2-yl-indazol-3-yl]pyrazol-1-yl]propoxy]ethoxy]-1-methyl-ethyl]methanesulfonate

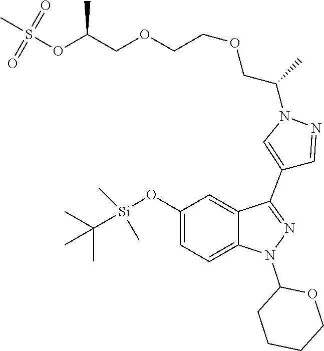 [SMILES: C[C@@H](COCCOC[C@H](C)n1cc(-c2nn(C3CCCCO3)c3ccc(O[Si](C)(C)C(C)(C)C)cc23)cn1)OS(C)(=O)=O]

To a solution of (2S)-1-[2-[(2S)-2-[4-[5-[tert-butyl(dimethyl)silyl]oxy-1-tetrahydropyran-2-yl-indazol-3-yl]pyrazol-1-yl]propoxy]ethoxy]propan-2-ol (114 mg, 0.20 mmol) in dichloro methane (2 mL) at 0° C. was added triethylamine (55 μL, 0.4 mmol) and methanesulfonyl chloride (20 μL, 0.26 mmol). The reaction mixture was stirred at 0° C. for 10 min then warmed to RT for 12 h. Water and dichloromethane were added and the layers were separated. The organic layer was washed with a saturated aqueous NaHCO₃ solution and with a saturated ammonium chloride solution. The organic layer was dried over anhydrous sodium sulfate, filtered and evaporated under reduced pressure to afford [(1S)-2-[2-[(2S)-2-[4-[5-[tert-butyl (dimethyl)silyl]oxy-1-tetrahydropyran-2-yl-indazol-3-yl]pyrazol-1-yl]propoxy]ethoxy]-1-methyl-ethyl]methanesulfonate as a yellow oil which was used in the next step without further purification.

LCMS method F: [M+H]⁺=637.5, t_{R}=3.46 min

### Preparation of Intermediate 550: (6S,13R)-6,13-dimethyl-19-(oxan-2-yl)-8,11,14-trioxa-4,5,19,20-tetraazatetracyclo[13.5.2.1^{2,5}.0^{18,21}]tricosa-1(20),2(23),3,15(22), 16,18(21)-hexaene

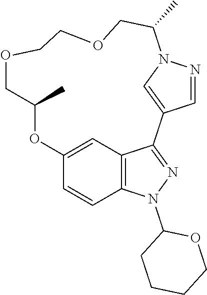 [SMILES: C[C@@H]1COCCOC[C@H](C)n2cc(cn2)-c2nn(C3CCCCO3)c3ccc(cc23)O1]

To a solution of cesium carbonate (187 mg, 5.75 mmol) in dry DMF (15 mL) at 60° C. was added dropwise a solution of [(1S)-2-[2-[(2S)-2-[4-[5-[tert-butyl(dimethyl)silyl]oxy-1-tetrahydro pyran-2-yl-indazol-3-yl]pyrazol-1-yl]propoxy]ethoxy]-1-methyl-ethyl] methanesulfonate (122 mg, 0.19 mmol) in dry DMF (5 mL). The reaction mixture was stirred at 60° C. for 1 h. The mixture was cooled to RT, filtered through a pad of Celite and washed with ethyl acetate. The organic layer was dried over anhydrous sodium sulfate, filtered and evaporated under reduced pressure. The residue was purified by silica gel column chromatography using diisopropyl ether/ethyl acetate 100/0 to 70/30 as eluent to afford (6S,13R)-6,13-dimethyl-19-(oxan-2-yl)-8,11,14-trioxa-4,5,19,20-tetraazatetracyclo[13.5.2.1^{2,5}.0^{18,21}]tricosa-1(20),2(23),3,15(22), 16,18(21)-hexaene as a colorless oil.

LCMS method F: [M+H]⁺=427.5, t_{R}=2.68 min

### Preparation of Example 93: (6S,13R)-6,13-dimethyl-8,11,14-trioxa-4,5,19,20-tetra azatetracyclo[13.5.2.1^{2,5}.0^{18,21}]tricosa-1(20),2(23),3,15(22),16,18(21)-hexaene

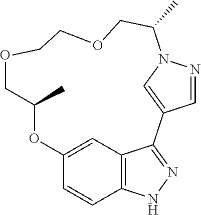 [SMILES: C[C@@H]1COCCOC[C@H](C)n2cc(cn2)-c2n[nH]c3ccc(cc23)O1]

To a solution of (6S,13R)-6,13-dimethyl-19-(oxan-2-yl)-8,11,14-trioxa-4,5,19,20-tetraazatetra cyclo[13.5.2.1^{2,5}.0^{18,21}]tricosa-1(20),2(23),3,15(22),16,18(21)-hexaene (27 mg, 0.063 mmol) in methanol (1.1 mL) and water (0.2 mL) was added p-toluenesulfonic acid monohydrate (60 mg, 0.317 mmol) and the reaction mixture was stirred at 65° C. for 16 h. The reaction mixture was diluted with dichloromethane and a saturated aqueous NaHCO₃ solution. The phases were separated and the aqueous layer was extracted with dichloromethane. The combined organic layer was washed brine, dried over anhydrous magnesium sulfate, filtered and evaporated under reduced pressure. The residue was purified by preparative TLC eluting with cyclohexane/ethyl acetate 40/60 to afford (6S,13R)-6,13-dimethyl-8,11,14-trioxa-4,5,19,20-tetraazatetracyclo [13.5.2.1^{2,5}.0^{18,21}]tricosa-1(20),2(23),3,15(22),16,18(21)-hexaene as a solid.

LCMS method F: [M+H]⁺=343.3, t_{R}=2.09 min

LCMS method G: [M+H]⁺=343.3, t_{R}=2.09 min

¹H NMR (400 MHz, CDCl₃) 8.48 (1H, s), 8.02 (1H, s), 7.92 (1H, d, J=2.3 Hz), 7.34 (1H, dd, J=8.8, 0.8 Hz), 7.09 (1H, dd, J=8.8, 2.0 Hz), 4.72-4.64 (1H, m), 4.54-4.43 (1H, m), 3.92 (1H, dd, J=10.4, 2.4 Hz), 3.88-3.83 (1H, m), 3.95-3.55 (6H, m), 1.65 (3H, d, J=6.6 Hz), 1.45 (3H, d, J=6.6 Hz) ppm.

### Example 94: (6R,12R)-6,12-dimethyl-9,13-dioxa-4,5,18,19,22-pentaazatetracyclo[12.5.2.1^{2,5}.0^{17,20}]docosa-1(19),2(22),3,14(21),15,17(20)-hexaene

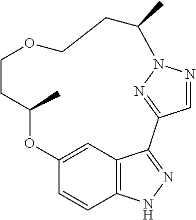 [SMILES: C[C@@H]1CCOCC[C@@H](C)n2ncc(n2)-c2n[nH]c3ccc(cc23)O1]

Example 94 is prepared according to the synthesis route described in general Scheme D.

### Preparation of Intermediate 551: (2S)-4-[tert-butyl(diphenyl)silyl]oxybutan-2-ol

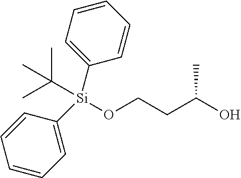 [SMILES: C[C@H](O)CCO[Si](c1ccccc1)(c1ccccc1)C(C)(C)C]

To a solution of (3S)-butane-1,3-diol (1.80 g, 20 mmol) and imidazole (2.72 g, 40 mmol) in THF (100 mL) was added tert-butyl(chloro)diphenylsilane (5.19 mL, 20 mmol). The reaction mixture was stirred at RT for 16 h. Solvents were evaporated under reduced pressure and the residue was partitioned between ethyl acetate and saturated aqueous ammonium chloride solution. The aqueous layer was extracted with ethyl acetate and the combined organic layers were washed with brine, dried over anhydrous anhydrous sodium sulfate, filtered and concentrated under reduced pressure. The residue was purified by silica gel column chromatography using cyclohexane/ethyl acetate 100/0 to 80/20 as eluent to afford (2S)-4-[tert-butyl(diphenyl)silyl]oxybutan-2-ol as a colorless oil.

LCMS method F: [M+H]⁺=329.2, t_{R}=3.33 min

### Preparation of Intermediate 552: tert-butyl-[(3R)-3-(4,5-dibromotriazol-2-yl) butoxy]-diphenyl-silane

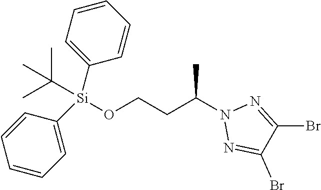 [SMILES: C[C@H](CCO[Si](c1ccccc1)(c1ccccc1)C(C)(C)C)n1nc(Br)c(Br)n1]

To a solution of 4,5-dibromo-2H-triazole (3.57 g, 15.73 mmol), (2S)-4-[tert-butyl(diphenyl) silyl]oxybutan-2-ol (5.17 g, 15.73 mmol) and triphenylphosphine (6.19 g, 23.60 mmol) in THF (50 mL) at 0° C. was added dropwise a solution of DIAD (4.63 mL, 23.60 mmol) in THF (25 mL). The reaction mixture was stirred at 0° C. for 5 min then RT for 3 h. Ethyl acetate and water were added and phases were separated. The aqueous layer was extracted with ethyl acetate. The combined organic layers were washed with saturated NaHCO₃ solution then with brine, dried over anhydrous sodium sulfate, filtered and evaporated under reduced pressure. The residue was purified by silica gel column chromatography using cyclohexane/ethyl acetate 100/0 to 90/10 as eluent to afford tert-butyl-[(3R)-3-(4,5-dibromotriazol-2-yl)butoxy]-diphenyl-silane as a colorless oil.

LCMS method M: [M+H]⁺=4.91, t_{R}=538.1 min

### Preparation of Intermediate 553: [(3R)-3-(4-bromotriazol-2-yl)butoxy]-tert-butyl-diphenyl-silane

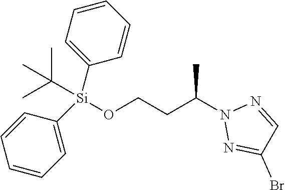 [SMILES: C[C@H](CCO[Si](c1ccccc1)(c1ccccc1)C(C)(C)C)n1ncc(Br)n1]

To a solution of tert-butyl-[(3R)-3-(4,5-dibromotriazol-2-yl)butoxy]-diphenyl-silane (7.50 g, 13.95 mmol) in THF (70 mL) at 0° C. was added dropwise isopropylmagnesium chloride lithium chloride complex solution (1.3 M solution in THF) (12.9 mL, 16.74 mmol). The reaction mixture was stirred at RT for 2 h. Saturated aqueous ammonium chloride solution and ethyl acetate were added and the phases were separated. The aqueous layer was extracted with ethyl acetate and the combined organic layers were washed with brine, dried over anhydrous sodium sulfate, filtered and evaporated under reduced pressure to afford [(3R)-3-(4-bromotriazol-2-yl)butoxy]-tert-butyl-diphenyl-silane as a pale yellow oil which was used in the next step without further purification.

LCMS method M: [M−Ph+H]⁺=380.3-382.2, t_{R}=5.16 min

### Preparation of Intermediate 554: (3R)-3-(4-bromotriazol-2-yl)butan-1-ol

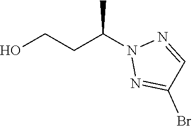

To a solution of [(3R)-3-(4-bromotriazol-2-yl)butoxy]-tert-butyl-diphenyl-silane (6.21 g, 13.55 mmol) in THF (70 mL) at 0° C. was added dropwise TBAF (1.0 M solution in THF) (16.3 mL, 16.26 mmol). The reaction mixture was stirred at RT for 16 h. Saturated aqueous ammonium chloride solution and ethyl acetate were added and the phases were separated. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent to afford (3R)-3-(4-bromotriazol-2-yl)butan-1-ol as a pale yellow oil.

LCMS method F: [M+H]⁺=220.1-222.1, t_{R}=1.63 min

### Preparation of Intermediate 555: 2-[(1R)-3-[(3S)-3-benzyloxybutoxy]-1-methyl-propyl]-4-bromo-triazole

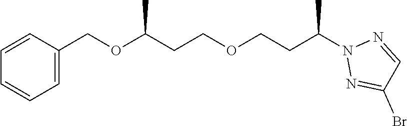 [SMILES: C[C@H](CCOCC[C@H](C)OCc1ccccc1)n1ncc(Br)n1]

To a 0° C. suspension of sodium hydride (60% dispersion in mineral oil) (527 mg, 13.177 mmol) in DMF (15 mL) was added dropwise a solution of (3R)-3-(4-bromotriazol-2-yl)butan-1-ol (1.45 g, 6.589 mmol) in DMF (25 mL). The reaction mixture was stirred at 0° C. for 5 min then to RT for 15 min. The reaction mixture was cooled to 0° C. and a solution of [(3S)-3-benzyloxybutyl] 4-methylbenzenesulfonate (3.30 g, 9.88 mmol) in DMF (25 mL) was added.

The resulting mixture was stirred at 0° C. for 5 min then to 65° C. for 3 h. The reaction mixture was cooled to RT and quenched with saturated aqueous ammonium chloride solution and diluted with ethyl acetate. The phases were separated and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 70/30 as eluent to afford 2-[(1R)-3-[(3S)-3-benzyloxybutoxy]-1-methyl-propyl]-4-bromo-triazole as a colorless oil.

LCMS method F: [M+H]⁺=382.3-384.3, t_{R}=3.18 min

### Preparation of Intermediate 556: [3-[2-[(1R)-3-[(3S)-3-benzyloxybutoxy]-1-methyl-propyl]triazol-4-yl]-1-tetrahydropyran-2-yl-indazol-5-yl]oxy-tert-butyl-dimethyl-silane

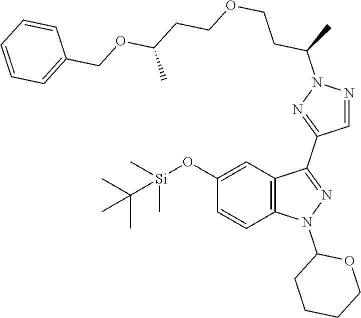 [SMILES: C[C@H](CCOCC[C@H](C)OCc1ccccc1)n1ncc(-c2nn(C3CCCCO3)c3ccc(O[Si](C)(C)C(C)(C)C)cc23)n1]

To a suspension of tert-butyl-dimethyl-[1-tetrahydropyran-2-yl-3-(4,4,5,5-tetramethyl-1,3,2-dioxaborolan-2-yl)indazol-5-yl]oxy-silane (1.30 g, 2.84 mmol), 2-[(1R)-3-[(3S)-3-benzyloxy butoxy]-1-methyl-propyl]-4-bromo-triazole (724 mg, 1.89 mmol), XPhos (90 mg, 0.189 mmol) and potassium phosphate tribasic (1.206 g, 5.68 mmol) in dioxane (20 mL) and water (1 mL) was added tetrakis(triphenylphosphine)-palladium(0) (110 mg, 0.095 mmol). The reaction mixture was stirred at 90° C. for 2 h. The reaction mixture was cooled to RT and partitioned between ethyl acetate and water. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 70/30 as eluent to afford [3-[2-[(1R)-3-[(3S)-3-benzyloxybutoxy]-1-methyl-propyl]triazol-4-yl]-1-tetrahydropyran-2-yl-indazol-5-yl]oxy-tert-butyl-dimethyl-silane as a pale yellow oil.

LCMS method M: [M+H]⁺=634.4, t_{R}=5.93 min

### Preparation of Intermediate 557: (2S)-4-[(3R)-3-[4-[5-[tert-butyl(dimethyl)silyl]oxy-1-tetrahydropyran-2-yl-indazol-3-yl]triazol-2-yl]butoxy] butan-2-ol

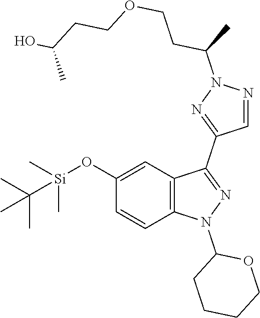 [SMILES: C[C@H](O)CCOCC[C@@H](C)n1ncc(-c2nn(C3CCCCO3)c3ccc(O[Si](C)(C)C(C)(C)C)cc23)n1]

To a solution of [3-[2-[(1R)-3-[(3S)-3-benzyloxybutoxy]-1-methyl-propyl]triazol-4-yl]-1-tetra hydropyran-2-yl-indazol-5-yl]oxy-tert-butyl-dimethyl-silane (577 mg, 0.910 mmol) in ethyl acetate (10 mL) under argon was added Pd(OH)₂/C (58 mg, 5% wt). The reaction mixture was stirred under hydrogen atmosphere for 20 h then heated to 50° C. for 24 h. The reaction mixture was cooled to RT and filtered over a Celite pad, washed with ethanol. The solvents were evaporated under reduced pressure to afford (2S)-4-[(3R)-3-[4-[5-[tert-butyl(dimethyl)silyl] oxy-1-tetrahydropyran-2-yl-indazol-3-yl]triazol-2-yl]butoxy] butan-2-ol as a pale grey oil which was used in the next step without further purification.

LCMS method M: [M+H]⁺=544.3, t_{R}=4.33 min

### Preparation of Intermediate 558: [(1S)-3-[(3R)-3-[4-[5-[tert-butyl(dimethyl)silyl]oxy-1-tetrahydropyran-2-yl-indazol-3-yl]triazol-2-yl]butoxy]-1-methyl-propyl] methanesulfonate

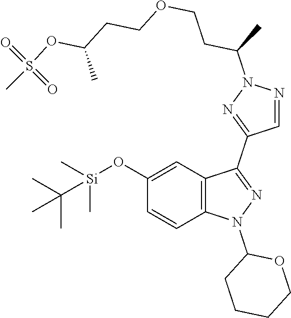 [SMILES: C[C@H](CCOCC[C@H](C)OS(C)(=O)=O)n1ncc(-c2nn(C3CCCCO3)c3ccc(O[Si](C)(C)C(C)(C)C)cc23)n1]

To a solution of (2S)-4-[(3R)-3-[4-[5-[tert-butyl(dimethyl)silyl]oxy-1-tetrahydropyran-2-yl-indazol-3-yl]triazol-2-yl]butoxy]butan-2-ol (439 mg, 0.807 mmol) and triethylamine (224 μL, 1.615 mmol) in dichloromethane (6 mL) at 0° C. was added a methanesulfonyl chloride (94 μL, 1.21 mmol) in dichloromethane (2.0 mL). The reaction mixture was stirred at RT for 3 h. Saturated aqueous ammonium chloride and dichloromethane were added and the phases were separated. The aqueous layer was extracted with dichloromethane and the combined organic extracts were washed with brine, dried over anhydrous sodium sulfate, filtered and evaporated under reduced pressure to afford [(1S)-3-[(3R)-3-[4-[5-[tert-butyl(dimethyl)silyl]oxy-1-tetrahydropyran-2-yl-indazol-3-yl]triazol-2-yl]butoxy]-1-methyl-propyl] methanesulfonate as a pale yellow oil which was used in the next step without further purification.

LCMS method M: [M+H]⁺=622.5, t_{R}=4.80 min

### Preparation of Intermediate 559: (6R,12R)-6,12-dimethyl-18-(oxan-2-yl)-9,13-dioxa-4,5,18,19,22-pentaazatetracyclo[12.5.2.1^{2,5}.0^{17,20}]docosa-1(19),2(22),3,14(21),15, 17(20)-hexaene

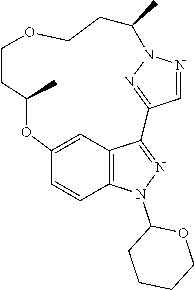 [SMILES: C[C@@H]1CCOCC[C@@H](C)n2ncc(n2)-c2nn(C3CCCCO3)c3ccc(cc23)O1]

To a suspension of cesium carbonate (530 mg, 1.628 mmol) in DMF (300 mL) at 85° C. was added dropwise [(1S)-3-[(3R)-3-[4-[5-[tert-butyl(dimethyl)silyl]oxy-1-tetrahydropyran-2-yl-indazol-3-yl]triazol-2-yl]butoxy]-1-methyl-propyl] methanesulfonate (549 mg, 0.807 mmol) in DMF (500 mL). The reaction mixture was stirred at 85° C. for 16 h. The reaction mixture was cooled to RT, filtered and concentrated under reduced pressure. The residue was partitioned between ethyl acetate and water. The aqueous layer was extracted with ethyl acetate and the combined organic extracts were washed with brine, dried over anhydrous sodium sulfate, filtered and evaporated under reduced pressure. The residue was purified by silica gel column chromatography using cyclohexane/(ethyl acetate/diisopropyl ether (3/1)) 100/0 to 70/30) as eluent to afford (6R,12R)-6,12-dimethyl-18-(oxan-2-yl)-9,13-dioxa-4,5,18,19,22-pentaazatetra cyclo[12.5.2.1^{2,5}.0^{17,20}]docosa-1(19),2(22),3,14(21),15,17(20)-hexaene as a white solid.

LCMS method F: [M+H]⁺=412.4, t_{R}=3.14 min

### Preparation of Example 94: (6R,12R)-6,12-dimethyl-9,13-dioxa-4,5,18,19,22-penta azatetracyclo[12.5.2.1^{2,5}.0^{17,20}]docosa-1(19),2(22),3,14(21),15,17(20)-hexaene

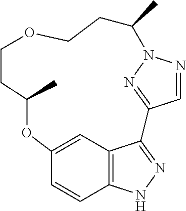 [SMILES: C[C@@H]1CCOCC[C@@H](C)n2ncc(n2)-c2n[nH]c3ccc(cc23)O1]

To a solution of (6R,12R)-6,12-dimethyl-18-(oxan-2-yl)-9,13-dioxa-4,5,18,19,22-pentaaza tetracyclo[12.5.2.1^{2,5}.0^{17,20}]docosa-1(19),2(22),3,14(21),15,17(20)-hexaene (65 mg, 0.158 mmol) in methanol (6.66 mL) and water (1.33 mL) was added p-toluenesulfonic acid monohydrate (150 mg, 0.790 mmol). The reaction mixture was stirred at 50° C. for 16 h. Solvents were concentrated under reduced pressure and the residue was partitioned between ethyl acetate and saturated aqueous NaHCO₃ solution. The aqueous layer was extracted with ethyl acetate and the combined organic layers were washed with brine, dried over anhydrous sodium sulfate, filtered and evaporated under reduced pressure. The residue was triturated with diisopropyl ether to afford (6R,12R)-6,12-dimethyl-9,13-dioxa-4,5,18,19,22-pentaazatetracyclo[12.5.2.1^{2,5}.0^{17,20}]docosa-1(19),2(22),3,14(21),15,17(20)-hexaene as a solid.

LCMS method J: [M+H]⁺=328.3, t_{R}=3.40 min

LCMS method N: [M+H]⁺=328.3, t_{R}=3.35 min

¹H NMR (400 MHz, d6 DMSO) δ 13.05 (s, 1H), 8.12 (s, 1H), 7.98 (d, J=2.3 Hz, 1H), 7.44 (d, J=9.1 Hz, 1H), 6.97 (dd, J=2.3, 9.1 Hz, 1H), 4.70-4.61 (m, 2H), 4.03-3.97 (m, 1H), 3.76-3.70 (m, 1H), 3.58-3.48 (m, 2H), 2.65-2.54 (m, 1H), 2.46-2.39 (m, 1H), 2.32-2.23 (m, 1H), 1.63 (d, J=6.8 Hz, 3H), 1.39 (d, J=6.0 Hz, 4H) ppm.

### Example 95: (13R)-6-methoxy-13-methyl-8,11,14-trioxa-4,5,19,20-tetraazatetracyclo [13.5.2.1^{2,5}.0^{18,21}]tricosa-1(20),2(23),3,15(22),16,18(21)-hexaene

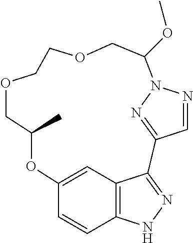 [SMILES: COC1COCCOC[C@@H](C)Oc2ccc3[nH]nc(c3c2)-c2cnn1n2]

Example 95 is prepared according to the synthesis route described in general Scheme D.

### Preparation of Intermediate 560: 2-(4-bromopyrazol-1-yl)-2-fluoro-acetic acid

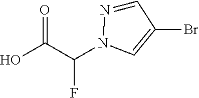 [SMILES: O=C(O)C(F)n1cc(Br)cn1]

To a solution of 4-bromo-1H-pyrazole (700 mg, 4.79 mmol) in DMF (35 mL) at 0° C. was added sodium hydride (60% dispersion in mineral oil) (421 mg, 10.54 mmol). The reaction mixture was stirred for 10 min at 0° C. then ethyl ethyl 2-bromo-2-fluoro-acetate (0.62 mL, 5.27 mmol) was added. The reaction mixture was stirred at RT for 2 h. The reaction mixture was quenched with and pH was adjusted at pH 1 with 1M hydrochloric acid solution. The aqueous layer was extracted with ethyl acetate. The organic layer was washed with water, brine, dried over anhydrous sodium sulfate, filtered and evaporated under reduced pressure to afford 2-(4-bromopyrazol-1-yl)-2-fluoro-acetic acid as a cream solid.

LCMS method F: [M+H]⁺=223.0-225.0, t_{R}=1.60 min

### Preparation of Intermediate 561: 2-(4-bromopyrazol-1-yl)-2-fluoro-ethanol

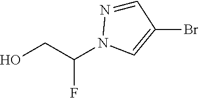

To a solution of 2-(4-bromopyrazol-1-yl)-2-fluoro-acetic acid (825 mg, 3.7 mmol) in THF (25 mL) was added borane dimethylsulfide (1 M solution in Me-THF) (7.4 mL, 7.4 mmol) at 0° C. The reaction mixture was stirred at RT overnight. The reaction mixture was quenched with methanol at 0° C., then water and ethyl acetate were added. The layers were separated and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent. to afford 2-(4-bromopyrazol-1-yl)-2-fluoro-ethanol as a colorless oil.

LCMS method F: [M+H]⁺=209.0-211.0, t_{R}=1.55 min

### Preparation of Intermediate 562: 2-[(2S)-2-benzyloxypropoxy]ethyl methane sulfonate

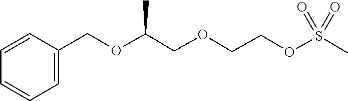 [SMILES: C[C@@H](COCCOS(C)(=O)=O)OCc1ccccc1]

To a solution of 2-[(2S)-2-benzyloxypropoxy]ethanol (460 mg, 2.19 mmol) in dichloromethane (10 mL) at 0° C. was added triethylamine (0.61 mL, 4.38 mmol) and methanesulfonyl chloride (0.22 mL, 2.85 mmol). The reaction mixture was stirred at RT for 2 h. Water and dichloromethane was added and the phases were separated. The organic layer was washed with a saturated aqueous NaHCO₃ solution and saturated ammonium chloride solution. The organic layer was dried over anhydrous sodium sulfate, filtered and evaporated under reduced pressure to afford 2-[(2S)-2-benzyloxypropoxy]ethyl methanesulfonate as a yellow liquid.

LCMS method F: [M+H]⁺=289.2, t_{R}=2.30 min

### Preparation of Intermediate 563: 1-[2-[2-[(2S)-2-benzyloxypropoxy]ethoxy]-1-fluoro-ethyl]-4-bromo-pyrazole

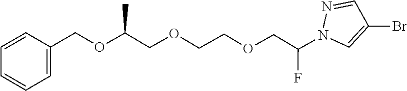 [SMILES: C[C@@H](COCCOCC(F)n1cc(Br)cn1)OCc1ccccc1]

To a solution of 2-(4-bromopyrazol-1-yl)-2-fluoro-ethanol (386 mg, 1.85 mmol) in dry DMF (4 mL) at 0° C. was added sodium hydride (60% dispersion in mineral oil) (111 mg, 2.78 mmol). The reaction mixture was stirred at RT for 10 min then, a solution of 2-[(2S)-2-benzyloxypropoxy]ethyl methanesulfonate (622 mg, 2.16 mmol) in dry DMF (4 mL) was added. The reaction mixture was stirred at RT overnight. The reaction mixture was quenched with water and extracted with ethyl acetate. The combined organic layers were washed with water and brine, dried over anhydrous sodium sulfate, filtered and evaporated under reduced pressure. The residue was purified by silica gel column chromatography using cyclohexane/ethyl acetate 100/0 to 80/20 as eluent to afford 1-[2-[2-[(2S)-2-benzyloxypropoxy] ethoxy]-1-fluoro-ethyl]-4-bromo-pyrazole as a colorless oil.

LCMS method F: [M+Na]⁺=423.2-425.2, t_{R}=2.88 min

### Preparation of Intermediate 564: [3-[1-[2-[2-[(2S)-2-benzyloxypropoxy]ethoxy]-1-fluoro-ethyl]pyrazol-4-yl]-1-tetrahydropyran-2-yl-indazol-5-yl]oxy-tert-butyl-dimethyl-silane

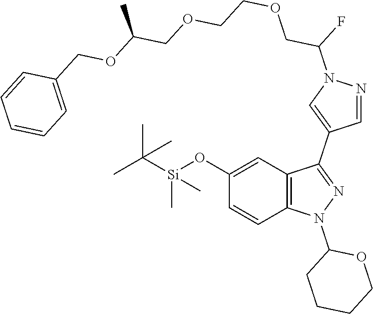 [SMILES: C[C@@H](COCCOCC(F)n1cc(-c2nn(C3CCCCO3)c3ccc(O[Si](C)(C)C(C)(C)C)cc23)cn1)OCc1ccccc1]

To a suspension of 1-[2-[2-[(2S)-2-benzyloxypropoxy]ethoxy]-1-fluoro-ethyl]-4-bromo-pyrazole (364 mg, 0.91 mmol) in dioxane (5.25 mL) and water (0.25 mL) was added tert-butyl-dimethyl-[1-tetrahydropyran-2-yl-3-(4,4,5,5-tetramethyl-1,3,2-dioxaborolan-2-yl)indazol-5-yl]oxy-silane (583 mg, 1.27 mmol) and potassium phosphate tribasic (579 mg, 2.73 mmol). The reaction mixture was purged under argon for 10 min and tetrakis(triphenylphosphine) palladium(0) (53 mg, 0.046 mmol) and Xphos (43 mg, 0.091 mmol) were added. The reaction mixture was heated under microwave irradiations for 2 h. The mixture was cooled to RT and filtered through a pad of Celite. The filtrate was diluted with water and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford [3-[1-[2-[2-[(2S)-2-benzyloxypropoxy]ethoxy]-1-fluoro-ethyl]pyrazol-4-yl]-1-tetrahydropyran-2-yl-indazol-5-yl]oxy-tert-butyl-dimethyl-silane as an orange oil.

LCMS method F: [M+H]⁺=653.4, t_{R}=3.76 min

### Preparation of Intermediate 565: (2S)-1-[2-[2-[4-[5-[tert-butyl(dimethyl)silyl]oxy-1-tetrahydropyran-2-yl-indazol-3-yl]pyrazol-1-yl]-2-fluoro-ethoxy]ethoxy]propan-2-ol

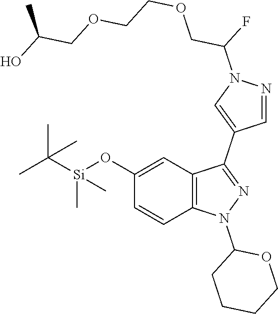 [SMILES: C[C@H](O)COCCOCC(F)n1cc(-c2nn(C3CCCCO3)c3ccc(O[Si](C)(C)C(C)(C)C)cc23)cn1]

To a solution of [3-[1-[2-[2-[(2S)-2-benzyloxypropoxy]ethoxy]-1-fluoro-ethyl]pyrazol-4-yl]-1-tetrahydropyran-2-yl-indazol-5-yl]oxy-tert-butyl-dimethyl-silane (491 mg, 0.75 mmol) in ethyl acetate (14 mL) at RT was added palladium on carbon 10 wt. % (50 mg). The reaction mixture was stirred under hydrogen atmosphere at RT overnight. The reaction mixture was filtered and the filtrate was concentrated under reduced pressure to afford (2S)-1-[2-[2-[4-[5-[tert-butyl(dimethyl)silyl]oxy-1-tetrahydropyran-2-yl-indazol-3-yl]pyrazol-1-yl]-2-fluoro-ethoxy]ethoxy]propan-2-ol as a pale black oil.

LCMS method F: [M+H]⁺=563.4, t_{R}=3.37 min

### Preparation of Intermediate 566: [(1S)-2-[2-[2-[4-[5-[tert-butyl(dimethyl)silyl]oxy-1-tetrahydropyran-2-yl-indazol-3-yl]pyrazol-1-yl]-2-fluoro-ethoxy]ethoxy]-1-methyl-ethyl]methanesulfonate

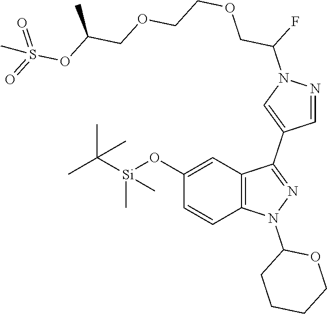 [SMILES: C[C@@H](COCCOCC(F)n1cc(-c2nn(C3CCCCO3)c3ccc(O[Si](C)(C)C(C)(C)C)cc23)cn1)OS(C)(=O)=O]

To a solution of (2S)-1-[2-[2-[4-[5-[tert-butyl(dimethyl)silyl]oxy-1-tetrahydropyran-2-yl-indazol-3-yl]pyrazol-1-yl]-2-fluoro-ethoxy]ethoxy]propan-2-ol (365 mg, 0.65 mmol) in dichloromethane (8 mL) were added triethylamine (0.18 mL, 1.3 mmol) and methanesulfonyl chloride (0.06 mL, 0.78 mmol). The reaction mixture was stirred at room temperature for 7 h. Additional triethylamine (0.09 mL, 0.65 mol) and methanesulfonyl chloride (0.03 mL, 0.39 mmol) were added and the reaction was stirred at RT overnight. The reaction mixture was diluted with water and extracted with dichloromethane. The combined organic layers were washed with a saturated ammonium chloride solution then with brine, dried over anhydrous sodium sulfate, filtered and evaporated under reduced pressure to afford [(1S)-2-[2-[2-[4-[5-[tert-butyl(dimethyl)silyl]oxy-1-tetrahydropyran-2-yl-indazol-3-yl]pyrazol-1-yl]-2-fluoro-ethoxy]ethoxy]-1-methyl-ethyl] methanesulfonate as a brown viscous oil.

LCMS method F: [M+H]⁺=641.5, t_{R}=3.52 min

### Preparation of Intermediate 567: (13R)-6-fluoro-13-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16, 18(21)-hexaene

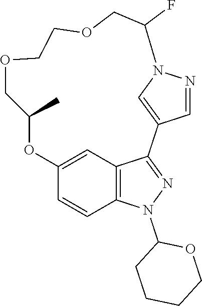 [SMILES: C[C@@H]1COCCOCC(F)n2cc(cn2)-c2nn(C3CCCCO3)c3ccc(cc23)O1]

To a suspension of cesium carbonate (585 mg, 1.8 mmol) in DMF (200 mL) at 85° C. was added dropwise [(1S)-2-[2-[2-[4-[5-[tert-butyl(dimethyl)silyl]oxy-1-tetrahydropyran-2-yl-indazol-3-yl]pyrazol-1-yl]-2-fluoro-ethoxy]ethoxy]-1-methyl-ethyl] methanesulfonate (385 mg, 0.6 mmol) in DMF (200 mL). The reaction mixture was stirred at 85° C. overnight. The reaction mixture was concentrated under reduced pressure and the resulting residue was diluted with water and extracted with ethyl acetate. The combined organic layers were washed with brine, dried over anhydrous sodium sulfate, filtered and evaporated under reduced pressure. The residue was purified by silica gel column chromatography using cyclohexane/(ethyl acetate/ethanol (3/1)) 100/0 to 80/20 as eluent to afford (13R)-6-fluoro-13-methyl-19-(oxan-2-yl)-8,11,14-trioxa-4,5,19,20-tetraazatetracyclo[13.5.2.1^{2,5}.0^{18,21}]tricosa-1(20),2(23),3, 15(22),16,18(21)-hexaene as a yellow viscous oil.

LCMS method F: [M+H]⁺=431.4, t_{R}=2.75/2.79 min

### Preparation of Example 95: (13R)-6-methoxy-13-methyl-8,11,14-trioxa-4,5,19,20-tetraazatetracyclo[13.5.2.1^{2,5}.0^{18,21}]tricosa-1(20),2(23),3,15(22),16,18(21)-hexaene

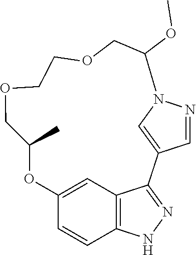 [SMILES: COC1COCCOC[C@@H](C)Oc2ccc3[nH]nc(c3c2)-c2cnn1c2]

To a solution of (13R)-6-fluoro-13-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene (126 mg, 0.29 mmol) in methanol (4.9 mL) and water (0.86 mL) was added p-toluenesulfonic acid monohydrate (278 mg, 1.47 mmol) and the reaction mixture was stirred at 65° C. for 5 h. The reaction mixture was diluted with dichloromethane and saturated aqueous NaHCO₃ solution. The phases were separated and the aqueous layer was extracted with dichloromethane. The combined organic layers were washed with brine, dried over anhydrous sodium sulfate, filtered and evaporated under reduced pressure. The residue was purified by silica gel column chromatography using dichloromethane/methanol 100/0 to 95/5 as eluent. The resulting solid was triturated in diisopropyl, filtered and dried to afford (13R)-6-methoxy-13-methyl-8,11,14-trioxa-4,5,19,20-tetraazatetracyclo[13.5.2.1^{2,5}.0^{18,21}]tricosa-1(20),2(23),3,15(22),16,18(21)-hexaene as a solid.

LCMS method F: [M+H]⁺=359.3, t_{R}=2.05 min

LCMS method G: [M+H]⁺=359.3, t_{R}=2.01 min

¹H NMR (400 MHz, CDCl₃) δ 8.92 (0.6H, s), 8.71 (0.4H, s), 8.19 (0.6H, d, J=1.8 Hz), 8.14 (0.6H, s), 8.04 (0.4H, s), 7.79 (0.4H, d, J=2.1 Hz), 7.38 (1H, m), 7.11 (1H, m), 5.52 (0.4H, m), 5.49 (0.6H, m), 4.59 (0.6H, m), 4.33 (0.4H, m), 4.26 (0.6H, dd, J=2.2, 10.9 Hz), 4.07 (0.4H, m), 4.01-3.52 (8H, m), 3.43 (1.7H, s), 3.33 (1.3H, s), 1.47 (1.3H, d, J=6.9 Hz), 1.43 (1.7H, d, J=6.9 Hz) ppm.

### Example 96: 8,11,14-trioxa-4,5,16,19,20-pentaazatetracyclo[13.5.2.1^{2,5}.0^{18,21}]tricosa-1(20),2(23),3,15(22),16,18(21)-hexaene

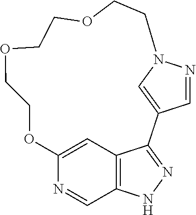 [SMILES: c1nn2cc1-c1n[nH]c3cnc(cc13)OCCOCCOCC2]

Example 96 is prepared according to the synthesis route described below.

### Preparation of Intermediate 568: 2-[2-[2-(2-benzyloxyethoxy)ethoxy]ethoxy]tetra hydropyran

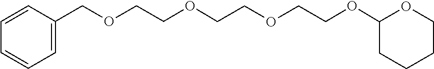 [SMILES: c1ccc(COCCOCCOCCOC2CCCCO2)cc1]

To a stirring solution of 2-(2-benzyloxyethoxy)ethanol (5 g, 25.5 mmol) in DMF (50 mL) was added portionwise sodium hydride (60% dispersion in mineral oil) (3.06 g, 76.5 mmol). The reaction mixture was stirred at RT for 30 min then 2-(2-bromoethoxy)tetrahydropyran (11.6 mL, 76.5 mmol) in DMF (50 mL) was added. The reaction mixture was stirred at 55° C. for 2 h. The reaction mixture was concentrated then diluted with water and ethyl acetate. The organic layer was washed with water, 10% aqueous lithium chloride solution and brine, dried over anhydrous magnesium sulfate, filtered and evaporated under reduced pressure. The residue was purified by silica gel column chromatography using cyclohexane/ethyl acetate 100/0 to 50/50 as eluent to afford 2-[2-[2-(2-benzyloxyethoxy)ethoxy]ethoxy]tetrahydropyran as a yellow oil.

¹H NMR (400 MHz, DMSO) 7.38-7.27 (m, 5H), 4.58 (t, J=3.2 Hz, 1H), 4.50 (s, 2H), 3.77-3.68 (m, 2H), 3.58-3.53 (m, 6H), 3.51-3.39 (m, 3H), 1.73-1.57 (m, 3H), 1.47-1.42 (m, 6H) ppm.

### Preparation of Intermediate 569: 2-[2-[2-(2-benzyloxyethoxy)ethoxy]ethoxy]tetra hydropyran

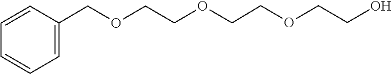 [SMILES: OCCOCCOCCOCc1ccccc1]

To a solution of 2-[2-[2-(2-benzyloxyethoxy)ethoxy]ethoxy]tetrahydropyran (11 g, 33.9 mmol) in methanol (80 mL) was added p-toluenesulfonic acid monohydrate (484 mg, 2.55 mmol). The reaction mixture was stirred at RT for 1 h. Methanol was partially evaporated under reduced pressure. The reaction was quenched with a saturated aqueous NaHCO₃ solution until pH basic and ethyl acetate was added. The layers were separated and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 0/100 as eluent to afford 2-[2-[2-(2-benzyloxyethoxy)ethoxy]ethoxy]tetrahydropyran as yellow oil.

LCMS method F: [M+H]⁺=241.2, t_{R}=1.81 min

### Preparation of Intermediate 570: 2-[2-[2-(2-benzyloxyethoxy)ethoxy]ethoxy]-4-methyl-5-nitro-pyridine

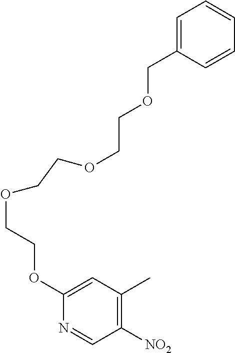 [SMILES: Cc1cc(OCCOCCOCCOCc2ccccc2)ncc1[N+](=O)[O-]]

To a mixture of 2-chloro-4-methyl-5-nitro-pyridine (1.14 g, 6.61 mmol) and 2-[2-(2-benzyloxyethoxy)ethoxy]ethanol (1.9 g, 7.93 mmol) in dry toluene (3.3 mL) under argon was added cesium carbonate (3.02 g, 9.25 mmol), Pd(dba)₂ (76 mg, 0.13 mmol) and rac-BINAP (247 mg, 0.40 mmol). The reaction mixture was stirred at 110° C. for 12 h. The reaction mixture was filtered over a pad of Celite, washed with water and ethyl acetate. The phases were separated and the aqueous layer was extracted with ethyl acetate. The organic layer was washed with brine, dried over anhydrous sodium sulfate, filtered and evaporated under reduced pressure. The residue was purified by silica gel column chromatography using cyclohexane/ethyl acetate 50/50 as eluent to afford 2-[2-[2-(2-benzyloxyethoxy)ethoxy] ethoxy]-4-methyl-5-nitro-pyridine as a red oil.

LCMS method F: [M+H]⁺=377.3, t_{R}=2.83 min

### Preparation of Intermediate 571: 6-[2-[2-(2-benzyloxyethoxy)ethoxy]ethoxy]-4-methyl-pyridin-3-amine

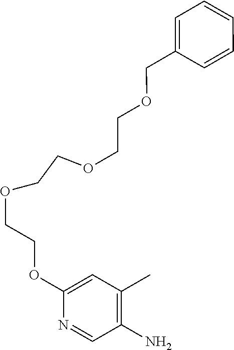 [SMILES: Cc1cc(OCCOCCOCCOCc2ccccc2)ncc1N]

To a solution of 2-[2-[2-(2-benzyloxyethoxy)ethoxy]ethoxy]-4-methyl-5-nitro-pyridine (2.26 g, 6.01 mmol) in ethanol (48 mL) and water (12 mL) was added ammonium chloride (3.18 g, 60.1 mmol) and iron (3.36 g, 60.1 mmol) under argon. The reaction mixture was stirred at 70° C. for 2 h. The reaction mixture was evaporated under reduced pressure. Dichloromethane and water were added and filtered under hydrophobic column. The phases were separated and the aqueous layer was extracted with dichloromethane, dried over anhydrous sodium sulfate, filtered and evaporated under reduced pressure. The residue was purified by silica gel column chromatography using dichloromethane/methanol 95/5 as eluent to afford 6-[2-[2-(2-benzyloxyethoxy)ethoxy]ethoxy]-4-methyl-pyridin-3-amine as a red oil.

LCMS method F: [M+H]⁺=347.3, t_{R}=1.66 min

### Preparation of Intermediate 572: N-[6-[2-[2-(2-benzyloxyethoxy)ethoxy]ethoxy]-4-methyl-3-pyridyl]acetamide

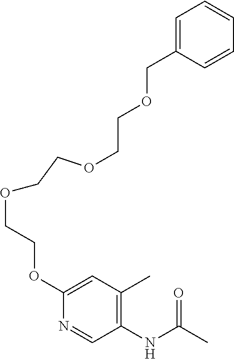 [SMILES: CC(=O)Nc1cnc(OCCOCCOCCOCc2ccccc2)cc1C]

To a solution of -[2-[2-(2-benzyloxyethoxy)ethoxy]ethoxy]-4-methyl-pyridin-3-amine (1.31 g, 3.79 mmol) in dichloromethane (7 mL) at 0° C. was added triethylamine (1.57 ml, 11.37 mmol) then acetic anhydride (640 μL, 6.81 mmol). The reaction mixture was stirred at RT for 3 h. A saturated aqueous NaHCO₃ solution and dichloromethane were added. The organic phases were separated and the aqueous was extracted with dichloromethane. The organic layer was washed with brine, dried over anhydrous sodium sulfate, filtered and evaporated under reduced pressure to afford N-[6-[2-[2-(2-benzyloxyethoxy)ethoxy]ethoxy]-4-methyl-3-pyridyl]acetamide as a brown oil.

LCMS method F: [M+H]⁺=389.3, t_{R}=2.14 min

### Preparation of Intermediate 573: 1-[5-[2-[2-(2-benzyloxyethoxy)ethoxy]ethoxy] pyrazolo[3,4-c]pyridin-1-yl]ethanone

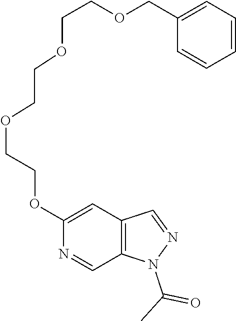 [SMILES: CC(=O)n1ncc2cc(OCCOCCOCCOCc3ccccc3)ncc21]

To stirred solution of N-[6-[2-[2-(2-benzyloxyethoxy)ethoxy]ethoxy]-4-methyl-3-pyridyl] acetamide (1.64 g, 4.23 mmol), potassium acetate (623 mg, 6.36 mmol) and acetic anhydride (1.84 mL, 19.5 mmol) in toluene (20 mL) at 75° C. was stirred for 10 min. Tert-butyl nitrite (1.57 mL, 6.36 mmol) was added and the reaction mixture was heated at 75° C. for 4 h. The reaction mixture was evaporated under reduced pressure and ethyl acetate and water was added. The phases were separated and the aqueous layer was extracted with ethyl acetate. The organic layer was washed with brine, dried over anhydrous sodium sulfate, filtered and evaporated under reduced pressure to afford 1-[5-[2-[2-(2-benzyloxyethoxy)ethoxy]ethoxy]pyrazolo[3,4-c]pyridin-1-yl]ethanone as a brown oil.

LCMS method F: [M+H]⁺=400.2, t_{R}=2.63 min

### Preparation of Intermediate 574: 5-[2-[2-(2-benzyloxyethoxy)ethoxy]ethoxy]-1H-pyrazolo[3,4-c]pyridine

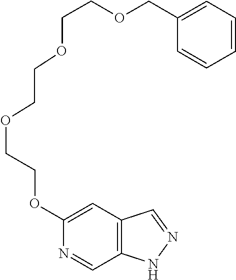 [SMILES: c1ccc(COCCOCCOCCOc2cc3cn[nH]c3cn2)cc1]

To a stirred suspension of 1-[5-[2-[2-(2-benzyloxyethoxy)ethoxy]ethoxy]pyrazolo[3,4-c] pyridin-1-yl]ethanone (2.34 g, 5.86 mmol) in methanol (30 mL) was added ammonia (4M solution in methanol) (10.3 mL, 41.05 mmol). The reaction mixture was stirred at RT for 2 h. The reaction mixture was evaporated under reduced pressure. The residue was purified by silica gel column chromatography using cyclohexane/ethyl acetate 100/0 to 0/100 as eluent to afford 5-[2-[2-(2-benzyloxyethoxy)ethoxy]ethoxy]-1H-pyrazolo[3,4-c]pyridine as an orange oil.

LCMS method F: [M+H]⁺=358.3, t_{R}=2.10 min

### Preparation of Intermediate 575: 5-[2-[2-(2-benzyloxyethoxy)ethoxy]ethoxy]-3-iodo-1H-pyrazolo[3,4-c]pyridine

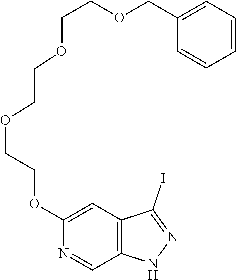 [SMILES: Ic1n[nH]c2cnc(OCCOCCOCCOCc3ccccc3)cc12]

To a solution of 5-[2-[2-(2-benzyloxyethoxy)ethoxy]ethoxy]-1H-pyrazolo[3,4-c]pyridine (569 mg, 1.59 mmol) in acetonitrile (3 mL) was added N-iodosuccinimide (430 mg, 1.91 mmol). The reaction mixture was stirred under microwave irradiations at 100° C. for 30 min. The reaction mixture was partially evaporated under reduced pressure and the residue was diluted with water and ethyl acetate. The phases were separated. The organic layer was washed with a saturated sodium thiosulfate solution. The aqueous layer was extracted with ethyl acetate and the combined organic layers were dried over anhydrous sodium sulfate, filtered and evaporated under reduced pressure to afford 5-[2-[2-(2-benzyloxyethoxy)ethoxy]ethoxy]-3-iodo-1H-pyrazolo[3,4-c]pyridine as a orange oil.

LCMS method F: [M+H]⁺=484.2, t_{R}=2.50 min

### Preparation of Intermediate 576: 5-[2-[2-(2-benzyloxyethoxy)ethoxy]ethoxy]-3-iodo-1-tetrahydropyran-2-yl-pyrazolo[3,4-c]pyridine

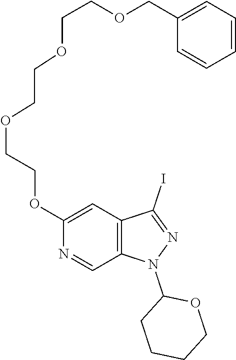 [SMILES: Ic1nn(C2CCCCO2)c2cnc(OCCOCCOCCOCc3ccccc3)cc12]

To a solution of 5-[2-[2-(2-benzyloxyethoxy)ethoxy]ethoxy]-3-iodo-1H-pyrazolo[3,4-c] pyridine (910 mg, 1.88 mmol) in dichloromethane (10 mL) was added p-toluenesulfonic acid monohydrate (179 mg, 0.94 mmol) and then 3,4-dihydro-2H-pyran (0.31 mL, 3.4 mmol). The reaction mixture was stirred at RT for 1 h then heated to 50° C. for 1 h. Methanesulfonic acid (0.036 mL, 0.56 mmol) was added and the reaction mixture and stirred at 50° C. for 2 h. The reaction mixture was diluted with a saturated NaHCO₃ solution of and dichloromethane. The phases were separated and the aqueous layer was extracted with dichloromethane. The combined organic layers were dried over anhydrous sodium sulfate, filtered and evaporated under reduced pressure. The residue was purified by silica gel column chromatography using cyclohexane/ethyl acetate 100/0 to 50/50 as eluent to afford 5-[2-[2-(2-benzyloxyethoxy) ethoxy]ethoxy]-3-iodo-1-tetrahydropyran-2-yl-pyrazolo[3,4-c]pyridine a pale yellow oil.

LCMS method F: [M+H]⁺=568.2, t_{R}=3.11 min

### Preparation of Intermediate 577: 2-[[4-[5-[2-[2-(2-benzyloxyethoxy)ethoxy]ethoxy]-1-tetrahydropyran-2-yl-pyrazolo[3,4-c]pyridin-3-yl]pyrazol-1-yl]methoxy]ethyl-trimethyl-silane

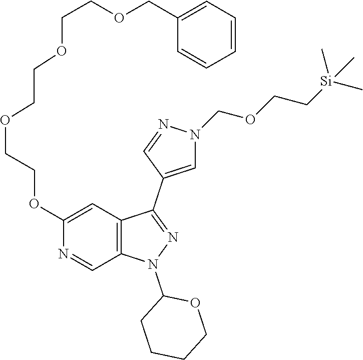 [SMILES: C[Si](C)(C)CCOCn1cc(-c2nn(C3CCCCO3)c3cnc(OCCOCCOCCOCc4ccccc4)cc23)cn1]

To a suspension of 5-[2-[2-(2-benzyloxyethoxy)ethoxy]ethoxy]-3-iodo-1-tetrahydropyran-2-yl-pyrazolo[3,4-c]pyridine (592 mg, 1.04 mmol) in dioxane (10 mL) and water (1 mL) was added trimethyl-[2-[[4-(4,4,5,5-tetramethyl-1,3,2-dioxaborolan-2-yl)pyrazol-1-yl]methoxy] ethyl]silane (440 mg, 1.36 mmol) and potassium phosphate tribasic (661 mg, 3.12 mmol). The reaction mixture was purged with argon for 15 min then tetrakis(triphenylphosphine) palladium(0) (60 mg, 0.05 mmol) and Xphos (50 mg, 0.10 mmol) were added. The reaction mixture was stirred at 100° C. for 12 h. The reaction mixture was evaporated under reduced pressure then ethyl acetate and water were added. The phases were separated and the aqueous layer was extracted with ethyl acetate. The organic layer was washed with brine, dried over anhydrous sodium sulfate, filtered and evaporated under reduced pressure. The residue was purified by silica gel column chromatography using cyclohexane/ethyl acetate 50/50 as eluent to afford 2-[[4-[5-[2-[2-(2-benzyloxyethoxy)ethoxy]ethoxy]-1-tetrahydropyran-2-yl-pyrazolo [3,4-c]pyridin-3-yl]pyrazol-1-yl]methoxy]ethyl-trimethyl-silane as a yellow oil.

LCMS method I: [M+H]⁺=638.3, t_{R}=2.78 min

### Preparation of Intermediate 578: 2-[2-[2-[1-tetrahydropyran-2-yl-3-[1-(2-trimethyl silylethoxymethyl)pyrazol-4-yl]pyrazolo[3,4-c]pyridin-5-yl]oxyethoxy]ethoxy]ethanol

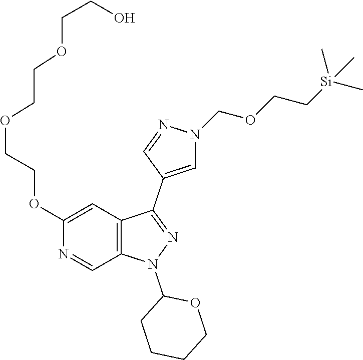 [SMILES: C[Si](C)(C)CCOCn1cc(-c2nn(C3CCCCO3)c3cnc(OCCOCCOCCO)cc23)cn1]

A solution of 2-[[4-[5-[2-[2-(2-benzyloxyethoxy)ethoxy]ethoxy]-1-tetrahydropyran-2-yl-pyrazolo[3,4-c]pyridin-3-yl]pyrazol-1-yl]methoxy]ethyl-trimethyl-silane (700 mg, 1.1 mmol) in EtOAc (10 mL) was purged with argon, then palladium hydroxide (13 mg, 0.11 mmol). The reaction mixture was stirred under hydrogen atmosphere at 60° C. for 24 hours. The stirring was stopped and the reaction mixture was filtered over celite and the filtrate was evaporated in vacuo to afford 2-[2-[2-[1-tetrahydropyran-2-yl-3-[1-(2-trimethylsilylethoxymethyl)pyrazol-4-yl] pyrazolo[3,4-c]pyridin-5-yl]oxyethoxy]ethoxy]ethanol as a yellow oil.

LCMS method F: [M+H]⁺=548.4, t_{R}=2.80 min

### Preparation of Intermediate 579: 2-[2-[2-[3-(1H-pyrazol-4-yl)-1-tetrahydropyran-2-yl-pyrazolo[3,4-c]pyridin-5-yl]oxyethoxy]ethoxy]ethanol

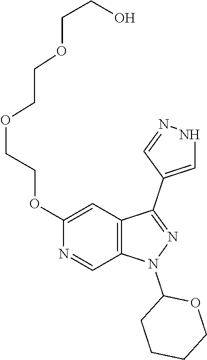 [SMILES: OCCOCCOCCOc1cc2c(-c3cn[nH]c3)nn(C3CCCCO3)c2cn1]

To a solution of 2-[2-[2-[1-tetrahydropyran-2-yl-3-[1-(2-trimethylsilylethoxymethyl)pyrazol-4-yl]pyrazolo[3,4-c]pyridin-5-yl]oxyethoxy]ethoxy]ethanol (510 mg, 0.93 mmol) in THF (4 mL) at RT was added TBAF (1 M solution in THF) (1.86 mL, 1.86 mmol). The reaction mixture was stirred at 60° C. for 12 h. TBAF (1 M solution in THF) (1.86 mL, 1.86 mmol) was added and the reaction mixture was stirred at RT for 48 h. Additional TBAF (1 M solution in THF) (0.9 mL, 0.9 mmol) was added and the reaction mixture was stirred at 60° C. for 4 h. The reaction mixture was quenched with saturated ammonium chloride solution and extracted with ethyl acetate. The combined organic layer was washed with brine, dried over anhydrous sodium sulfate and evaporated under reduced pressure. The residue was purified by silica gel column chromatography using dichloromethane/methanol 90/10 as eluent to afford 2-[2-[2-[3-(1H-pyrazol-4-yl)-1-tetrahydropyran-2-yl-pyrazolo[3,4-c]pyridin-5-yl]oxyethoxy]ethoxy]ethanol as a yellow oil.

LCMS method F: [M+H]⁺=418.2, t_{R}=1.86 min

### Preparation of Intermediate 580: 19-(oxan-2-yl)-8,11,14-trioxa-4,5,16,19,20-penta azatetracyclo[13.5.2.1^{2,5}.0^{18,21}]tricosa-1(20),2(23),3,15(22),16,18(21)-hexaene

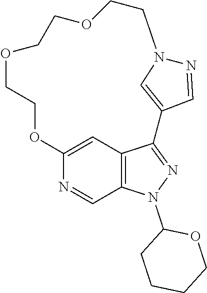 [SMILES: c1nn2cc1-c1nn(C3CCCCO3)c3cnc(cc13)OCCOCCOCC2]

To a solution of 2-[2-[2-[3-(1H-pyrazol-4-yl)-1-tetrahydropyran-2-yl-pyrazolo[3,4-c]pyridin-5-yl]oxyethoxy]ethoxy]ethanol (20 mg, 0.048 mmol) and triethylamine (27 μL, 0.192 mmol) in dichloromethane (1.4 mL) at 0° C. was added methanesulfonyl chloride (11 μL, 0.144 mmol). The reaction mixture was stirred at RT overnight. The reaction mixture was diluted with water and extracted with dichloromethane. The combined organic layer was dried over anhydrous magnesium sulfate, filtered and evaporated under reduced pressure. The residue was purified by preparative TLC eluting with diisopropylether/ethyl acetate/ethanol 6/3/1 to afford 19-(oxan-2-yl)-8,11,14-trioxa-4,5,16,19,20-pentaazatetracyclo[13.5.2.1^{2,5}.0^{18,21}]tricosa-1(20), 2(23),3,15(22),16,18(21)-hexaene as a white solid.

LCMS method F: [M+H]⁺=400.1, t_{R}=2.05 min

### Preparation of Example 96: 8,11,14-trioxa-4,5,16,19,20-pentaazatetracyclo[13.5.2.1^{2,5}.0^{18,21}]tricosa-1(20),2(23),3,15(22),16,18(21)-hexaene

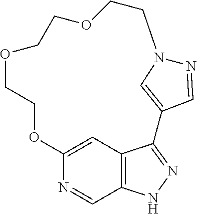 [SMILES: c1nn2cc1-c1n[nH]c3cnc(cc13)OCCOCCOCC2]

To a solution of 19-(oxan-2-yl)-8,11,14-trioxa-4,5,16,19,20-pentaazatetracyclo[13.5.2.1^{2,5}.0^{18,21}]tricosa-1(20),2(23),3,15(22),16,18(21)-hexaene (8 mg, 0.020 mmol) in methanol (350 μL) and water (50 μL) was added p-toluenesulfonic acid monohydrate (19 mg, 0.100 mmol) and the reaction mixture was stirred at 65° C. for 6 h. The reaction mixture was diluted with dichloromethane and saturated aqueous NaHCO₃ solution. The phases were separated and the aqueous layer was extracted with dichloromethane. The combined organic layer was washed brine, dried over anhydrous sodium sulfate, filtered and evaporated under reduced pressure. The residue was purified by preparative TLC using dichloromethane/methanol 95/5 as an eluent to afford 8,11,14-trioxa-4,5,16,19,20-pentaazatetracyclo[13.5.2.1^{2,5}.0^{18,21}]tricosa-1(20),2(23), 3,15(22),16,18(21)-hexaene as a solid.

LCMS method F: [M+H]⁺=316.3, t_{R}=1.44 min

LCMS method G: [M+H]⁺=316.3, t_{R}=1.48 min

¹H NMR (400 MHz, CDCl₃) 10.27 (1H, br. s), 8.65 (1H, d, J=1.2 Hz), 8.61 (1H, d, J=0.5 Hz), 8.04 (1H, d, J=1.2 Hz), 8.03 (1H, d, J=0.5 Hz), 4.58-4.54 (2H, m), 4.51-4.47 (2H, m), 3.98-3.94 (2H, m), 3.89-3.85 (2H, m), 3.77-3.71 (4H, m) ppm.

### Example 97: (8S,12R)-8,12-dimethyl-9,13-dioxa-4,5,18,19-tetraazatetracyclo[12.5.2.1^{2,5}.0^{17,20}]docosa-1(19),2(22)_,314(21),15,17(20)-hexaene

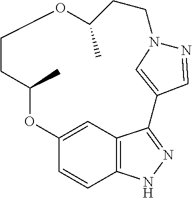 [SMILES: C[C@@H]1CCO[C@@H](C)CCn2cc(cn2)-c2n[nH]c3ccc(cc23)O1]

Example 97 is prepared according to the synthesis route described in general Scheme E.

### Preparation of Intermediate 581: methyl (3S)-3-tetrahydropyran-2-yloxybutanoate

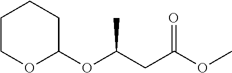

To a solution of (S)-methyl 3-hydroxybutanoate (4.72 g, 40 mmol) and DHP (5.47 mL, 60 mmol) in diethyl ether (40 mL) at 0° C. was added p-toluenesulfonic acid monohydrate (761 mg, 4 mmol). The resulting reaction mixture was stirred at 0° for 4 h. The residue was diluted with saturated aqueous NaHCO₃ solution and extracted with diethyl ether. The combined organic layers were dried over anhydrous sodium sulfate, filtered and evaporated under reduced pressure. The residue was purified by silica gel column chromatography using cyclohexane/ethyl acetate 100/0 to 95/5 as eluent to afford methyl (3S)-3-tetrahydropyran-2-yloxybutanoate as a colorless liquid.

¹H NMR (400 MHz, CDCl₃) δ 4.76-4.71 (t, J=3.0 Hz, OH), 4.31-4.17 (m, 1H), 3.96-3.83 (m, 1H), 3.71-3.69 (m, 3H), 3.57-3.52 (m, 1H), 3.51-3.47 (m, 1H), 2.71-2.60 (dd, J=6.9, 15.1 Hz, 1H), 2.46-2.42 (dd, J=6.0, 15.6 Hz, 1H), 1.90-1.49 (m, 7H), 1.32-1.22 (m, 3H) ppm.

### Preparation of Intermediate 582: (3S)-3-tetrahydropyran-2-yloxybutan-1-ol

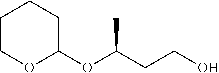

To a solution of LiAlH₄ (1M solution in THF) (32.84 mL, 32.84 mmol) at 0° C. was added dropwise methyl (3S)-3-tetrahydropyran-2-yloxybutanoate (6.64 g, 32.84 mmol) in anhydrous THF (66 mL). The reaction mixture was stirred at 0° C. for 1 h. The reaction mixture was quenched at 0° C. by addition of water (1.24 mL) in 10 mL of THF, 15% aqueous sodium hydroxide solution (1.24 mL) and water (3.72 mL). After stirring at RT for 30 min, the precipitate was filtered through a pad of Celite and washed with ertgyl acetate. The filtrate was concentrated under reduced pressure and the residue was purified by silica gel column chromatography using cyclohexane/ethyl acetate 100/0 to 50/50 as eluent to afford (3S)-3-tetrahydropyran-2-yloxybutan-1-ol as a colorless liquid.

¹H NMR (400 MHz, CDCl₃) δ 4.74-4.59 (m, 1H), 4.17-4.04 (m, 1H), 4.01-3.92 (m, 2H), 3.91-3.80 (m, 1H), 3.77-3.68 (m, 1H), 3.56-3.50 (m, 1H), 1.86-1.50 (m, 8H), 1.33-1.19 (m, 3H) ppm.

### Preparation of Intermediate 583: 2-[(1S)-3-benzyloxy-1-methyl-propoxy]tetrahydro pyran

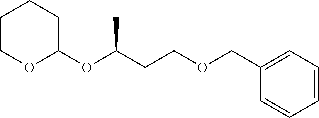 [SMILES: C[C@@H](CCOCc1ccccc1)OC1CCCCO1]

To a suspension of sodium hydride (60% dispersion in mineral oil) (780 mg, 19.5 mmol) in anhydrous DMF (5 mL) at 0° C. was added dropwise (3S)-3-tetrahydropyran-2-yloxybutan-1-ol (2.61 g, 15 mmol) in anhydrous DMF (5 mL). The reaction mixture was stirred at 0° C. foe 20 min then benzyl bromide (2.68 mL, 22.5 mmol) in DMF (5 mL) was added dropwise. The resulting reaction mixture was stirred at RT overnight. The reaction mixture was quenched by addition of water then concentrated under reduced pressure. The residue was diluted with saturated aqueous sodium chloride solution and extracted with ethyl acetate. The combined organic layers were dried over anhydrous sodium sulfate, filtered and concentrated under reduced pressure. The residue was purified by silica gel column chromatography using cyclohexane/ethyl acetate 100/0 to 90/10 as eluent to afford 2-[(1S)-3-benzyloxy-1-methyl-propoxy]tetrahydropyran as a colorless oil.

LCMS method F: [M+Na]⁺=287.2, t_{R}=2.89 min

### Preparation of Intermediate 584: (2S)-4-benzyloxybutan-2-ol

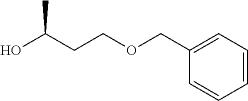

To a solution of 2-[(1S)-3-benzyloxy-1-methyl-propoxy]tetrahydropyran (3.86 g, 14.6 mmol) in methanol (35 mL) and water (5 mL) was added p-toluenesulfonic acid monohydrate (13.89 g, 73.02 mmol). The reaction mixture was stirred at RT overnight then at 60° C. for 24 h. The reaction mixture was concentrated under reduced pressure and the residue was neutralized by addition of saturated aqueous NaHCO₃ solution. The residue was diluted with ethyl acetate. The phases were separated and the aqueous phase was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate using 100/0 to 70/30 as eluent to afford (2S)-4-benzyloxybutan-2-ol as a colorless oil.

LCMS method F: [M+H]⁺=181.2, t_{R}=2.00 min

### Preparation of Intermediate 585: [(3S)-3-tetrahydropyran-2-yloxybutyl]-4-methyl benzenesulfonate

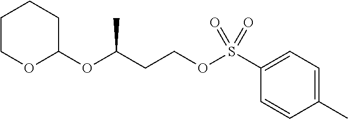 [SMILES: Cc1ccc(S(=O)(=O)OCC[C@H](C)OC2CCCCO2)cc1]

To a solution of (3S)-3-tetrahydropyran-2-yloxybutan-1-ol (871 mg, 5.00 mmol), triethylamine (1.394 mL, 10 mmol) and DMAP (31 mg, 0.25 mmol) in dichloromethane (10 mL) was added portionwise at 0° C. p-toluenesulfonyl chloride (1.24 g, 6.5 mmol). The reaction mixture was stirred at RT overnight. The reaction mixture was diluted with saturated ammonium chloride solution and extracted with dichloromethane. The combined organic layers were washed with sodium chloride solution, dried over anhydrous sodium sulfate, filtered and concentrated under reduced pressure. The residue was purified by silica gel column chromatography using cyclohexane/ethyl acetate 100/0 to 90/10 as eluent to afford [(3S)-3-tetrahydropyran-2-yloxy butyl]-4-methylbenzenesulfonate as a colorless oil.

LCMS method F: [M+H]⁺=not detected, t_{R}=2.83 min

### Preparation of Intermediate 586: 2-[(1S)-3-[(1S)-3-benzyloxy-1-methyl-propoxy]-1-methyl-propoxy]tetrahydropyran

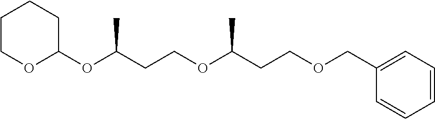 [SMILES: C[C@@H](CCOCc1ccccc1)OCC[C@H](C)OC1CCCCO1]

To a suspension of sodium hydride (60% dispersion in mineral oil) (450 mg, 11.25 mmol) in anhydrous DMF (5 mL) at 0° C. was added dropwise (2S)-4-benzyloxybutan-2-ol (1.352 g, 7.5 mmol) in DMF (5 mL). After 30 min at RT, [(3S)-3-tetrahydropyran-2-yloxybutyl]-4-methyl benzenesulfonate (1.642 g, 5 mmol) in DMF (5 mL) was added dropwise. The reaction mixture was stirred at 60° C. for 1 h. The reaction mixture was quenched by addition of water then concentrated under reduced pressure. The residue was diluted with saturated sodium chloride solution and extracted with ethyl acetate. The combined organic layers were dried over anhydrous sodium sulfate, filtered and concentrated under reduced pressure. The residue was purified by silica gel column chromatography using cyclohexane/ethyl acetate 100/0 to 90/10 as eluent to afford 2-[(1S)-3-[(1S)-3-benzyloxy-1-methyl-propoxy]-1-methyl-propoxy]tetra hydropyran as a colorless oil.

LCMS method F: [M+Na]⁺=359.2, t_{R}=3.14 min

### Preparation of Intermediate 587: (2S)-4-[(1S)-3-benzyloxy-1-methyl-propoxy]butan-2-ol

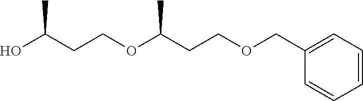 [SMILES: C[C@H](O)CCO[C@@H](C)CCOCc1ccccc1]

To a solution of 2-[(1S)-3-[(1S)-3-benzyloxy-1-methyl-propoxy]-1-methyl-propoxy]tetra hydropyran (880 mg, 2.62 mmol) in methanol (35 mL) and water (5 mL) was added p-toluenesulfonic acid monohydrate (2.487 g, 13.08 mmol). The reaction mixture was stirred at RT overnight then at 60° C. for 24 h. The reaction mixture was concentrated under reduced pressure and the residue was neutralized by addition of saturated aqueous NaHCO₃ solution. The residue was diluted with ethyl acetate. The phases were separated and the aqueous phase was extracted with ethyl acetate. The combined organic layers were washed with brine, dried over sodium sulfate, filtered and evaporated under reduced pressure. The residue was purified by silica gel column chromatography using cyclohexane/ethyl acetate 100/0 to 70/30 as eluent to afford (2S)-4-[(1S)-3-benzyloxy-1-methyl-propoxy]butan-2-ol as a colorless oil.

LCMS method F: [M+H]⁺=253.2, t_{R}=2.35 min

### Preparation of Intermediate 588: [(1S)-3-[(1S)-3-benzyloxy-1-methyl-propoxy]-1-methyl-propyl]methanesulfonate

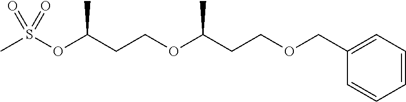 [SMILES: C[C@@H](CCOCc1ccccc1)OCC[C@H](C)OS(C)(=O)=O]

To a solution of (2S)-4-[(1S)-3-benzyloxy-1-methyl-propoxy]butan-2-ol (635 mg, 2.52 mmol) and triethylamine (702 μL, 5.03 mmol) in dichloromethane (5 mL) at 0° C. was added methanesulfonyl chloride (292 μL, 3.78 mmol). The reaction mixture was stirred at RT for 2 h. The reaction mixture was diluted with water and extracted with dichloromethane. The combined organic layers were washed with sodium chloride solution, dried over anhydrous sodium sulfate, filtered and concentrated under reduced pressure to afford [(1S)-3-[(1S)-3-benzyloxy-1-methyl-propoxy]-1-methyl-propyl]methanesulfonate as a yellow oil which was used in the next step without further purification.

LCMS method F: [M+H]⁺=331.2, t_{R}=2.70 min

### Preparation of Intermediate 589: 2-[[4-[5-[(1R)-3-[(1S)-3-benzyloxy-1-methyl-propoxy]-1-methyl-propoxy]-1-tetrahydropyran-2-yl-indazol-3-yl]pyrazol-1-yl] methoxy]ethyl-trimethyl-silane

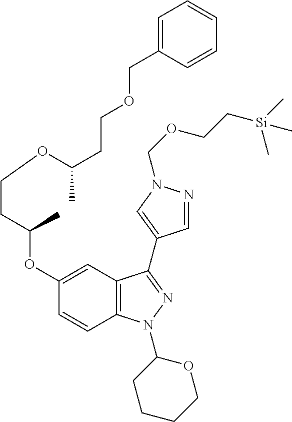 [SMILES: C[C@H](CCO[C@@H](C)CCOCc1ccccc1)Oc1ccc2c(c1)c(-c1cnn(COCC[Si](C)(C)C)c1)nn2C1CCCCO1]

To a solution of 1-tetrahydropyran-2-yl-3-[1-(2-trimethylsilylethoxymethyl)pyrazol-4-yl] indazol-5-ol (950 mg, 2.29 mmol) in DMF (20 mL) was added cesium carbonate (1.12 g, 3.44 mmol) and [(1S)-3-[(1S)-3-benzyloxy-1-methyl-propoxy]-1-methyl-propyl] methanesulfonate (833 mg, 2.52 mmol). The reaction mixture was stirred at 70° C. overnight. The reaction mixture was filtered then concentrated under reduced pressure. The residue was dissolved in ethyl acetate and water was added. The phases were separated and the aqueous phase was extracted with ethyl acetate. The combined organic layers were washed with brine, dried over sodium sulfate, filtered and evaporated under reduced pressure. The residue was purified by silica column chromatography using dichloromethane/ethyl acetate 100/0 to 50/50 as eluent to afford 2-[[4-[5-[(1R)-3-[(1S)-3-benzyloxy-1-methyl-propoxy]-1-methyl-propoxy]-1-tetrahydro pyran-2-yl-indazol-3-yl]pyrazol-1-yl]methoxy]ethyl-trimethyl-silane as a yellow oil.

LCMS method F: [M+H]⁺=649.4, t_{R}=3.76 min

### Preparation of Intermediate 590: (3S)-3-[(3R)-3-[1-tetrahydropyran-2-yl-3-[1-(2-trimethylsilylethoxymethyl) pyrazol-4-yl]indazol-5-yl]oxybutoxy]butan-1-ol

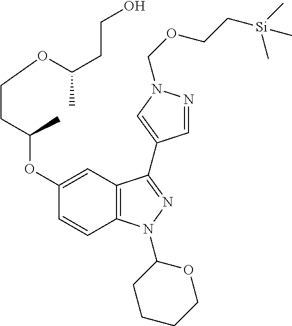 [SMILES: C[C@H](CCO[C@@H](C)CCO)Oc1ccc2c(c1)c(-c1cnn(COCC[Si](C)(C)C)c1)nn2C1CCCCO1]

To a solution of 2-[[4-[5-[(1R)-3-[(1S)-3-benzyloxy-1-methyl-propoxy]-1-methyl-propoxy]-1-tetrahydropyran-2-yl-indazol-3-yl]pyrazol-1-yl]methoxy]ethyl-trimethyl-silane (1.32 g, 2.03 mmol) in ethanol (10 mL) at RT was added palladium hydroxide on carbon (132 mg). The reaction mixture was stirred under hydrogen atmosphere at 50° C. for 36 h. The reaction mixture was filtered and the filtrate was concentrated under reduced pressure. The residue was purified by silica gel column chromatography using dichloromethane/ethyl acetate 100/0 to 20/80 as eluent to afford (3S)-3-[(3R)-3-[1-tetrahydropyran-2-yl-3-[1-(2-trimethylsilylethoxymethyl) pyrazol-4-yl]indazol-5-yl]oxybutoxy]butan-1-ol as a colorless oil.

LCMS method F: [M+H]⁺=559.3, t_{R}=3.22 min

### Preparation of Intermediate 591: (3S)-3-[(3R)-3-[3-(1H-pyrazol-4-yl)-1-tetrahydro pyran-2-yl-indazol-5-yl]oxybutoxy]butan-1-ol

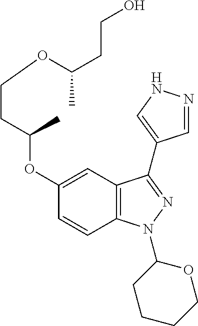 [SMILES: C[C@H](CCO[C@@H](C)CCO)Oc1ccc2c(c1)c(-c1cn[nH]c1)nn2C1CCCCO1]

To a solution of (3S)-3-[(3R)-3-[1-tetrahydropyran-2-yl-3-[1-(2-trimethylsilylethoxymethyl) pyrazol-4-yl]indazol-5-yl]oxybutoxy]butan-1-ol (970 mg, 1.74 mmol) in THF (10 mL) at RT was added TBAF (1 M solution in THF) (3.47 mL, 3.47 mmol). The reaction mixture was stirred at 60° C. overnight. The reaction mixture was quenched with saturated ammonium chloride solution and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 50/50 as eluent to afford (3S)-3-[(3R)-3-[3-(1H-pyrazol-4-yl)-1-tetrahydropyran-2-yl-indazol-5-yl] oxybutoxy]butan-1-ol as a colorless oil.

LCMS method F: [M+H]⁺=429.2, t_{R}=2.35 min

### Preparation of Intermediate 592: [(3S)-3-[(3R)-3-[3-(1-methylsulfonylpyrazol-4-yl)-1-tetrahydropyran-2-yl-indazol-5-yl]oxybutoxy]butyl] methanesulfonate

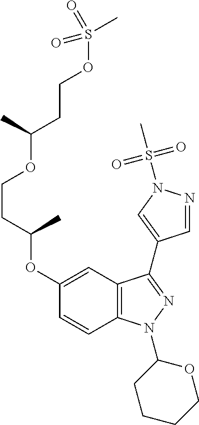 [SMILES: C[C@H](CCO[C@@H](C)CCOS(C)(=O)=O)Oc1ccc2c(c1)c(-c1cnn(S(C)(=O)=O)c1)nn2C1CCCCO1]

To a solution of (3S)-3-[(3R)-3-[3-(1H-pyrazol-4-yl)-1-tetrahydropyran-2-yl-indazol-5-yl] oxybutoxy]butan-1-ol (210 mg, 0.49 mmol) and triethylamine (273 μL, 1.96 mmol) in dichloromethane (5 mL) at 0° C. was added methanesulfonyl chloride (114 μL, 1.47 mmol). The reaction mixture was stirred at RT overnight. The reaction mixture was diluted with brine and extracted with dichloromethane. The combined organic layers were dried over anhydrous sodium sulfate, filtered and evaporated under reduced pressure to afford [(3S)-3-[(3R)-3-[3-(1-methylsulfonylpyrazol-4-yl)-1-tetrahydropyran-2-yl-indazol-5-yl]oxybutoxy]butyl]methane sulfonate as a yellow oil which was used in the next step without further purification.

LCMS method F: [M+H]⁺=585.2, t_{R}=2.88 min

### Preparation of Intermediate 593: (8S,12R)-8,12-dimethyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene

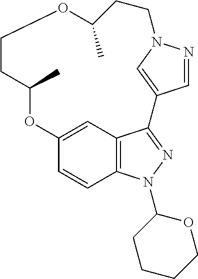 [SMILES: C[C@@H]1CCO[C@@H](C)CCn2cc(cn2)-c2nn(C3CCCCO3)c3ccc(cc23)O1]

To a suspension of cesium carbonate (797 mg, 2.45 mmol) in acetonitrile (125 mL) at 80° C. was added dropwise [(3S)-3-[(3R)-3-[3-(1-methylsulfonylpyrazol-4-yl)-1-tetrahydropyran-2-yl-indazol-5-yl]oxy butoxy]butyl]methanesulfonate (286 mg, 0.49 mmol) in acetonitrile (120 mL). The reaction mixture was stirred at 80° C. overnight. The reaction mixture was concentrated in vacuo then diluted with ethyl acetate and water. The phases were separated and the aqueous phase was extracted with ethyl acetate. The combined organic layers were washed with brine, dried over sodium sulfate, filtered and evaporated under reduced pressure. The residue was purified by silica gel column chromatography cyclohexane/(ethyl acetate/ethanol 3/1)) 100/0 to 80/20 as eluent to afford (8S,12R)-8,12-dimethyl-18-(oxan-2-yl)-9,13-dioxa-4,5,18,19-tetraazatetracyclo[12.5.2.1^{2,5}.0^{17,20}]docosa-1(19),2(22),3,14(21),15,17(20)-hexaene as a colorless oil.

LCMS method F: [M+H]⁺=411.2, t_{R}=2.84 min

### Preparation of Example 97: (8S,12R)-8,12-dimethyl-9,13-dioxa-4,5,18,19-tetraaza tetracyclo[12.5.2.1^{2,5}.0^{17,20}]docosa-1(19),2(22),3,14(21),15,17(20)-hexaene

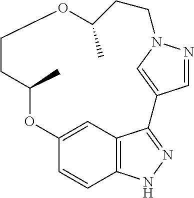 [SMILES: C[C@@H]1CCO[C@@H](C)CCn2cc(cn2)-c2n[nH]c3ccc(cc23)O1]

To a solution of (8S,12R)-8,12-dimethyl-18-(oxan-2-yl)-9,13-dioxa-4,5,18,19-tetraazatetra cyclo[12.5.2.1^{2,5}.0^{17,20}]docosa-1(19),2(22),3,14(21),15,17(20)-hexaene (190 mg, 0.46 mmol) in methanol (7 mL) and water (1 mL) was added p-toluenesulfonic acid monohydrate (440 mg, 2.31 mmol). The reaction mixture was stirred at 65° C. overnight. The reaction mixture was concentrated under vacuo and the residue was neutralized by addition of saturated aqueous NaHCO₃ solution. The residue was diluted with ethyl acetate. The phases were separated and the aqueous phase was extracted with ethyl acetate. The combined organic layers were washed with brine, dried over sodium sulfate, filtered and evaporated under reduced pressure. The residue was recrystallized with acetonitrile, filtered and dried to afford (8S,12R)-8,12-dimethyl-9,13-dioxa-4,5,18,19-tetraazatetracyclo[12.5.2.1^{2,5}.0^{17,20}]docosa-1(19),2(22),3,14(21),15, 17(20)-hexaene as a solid.

LCMS method F: [M+H]⁺=327.3, t_{R}=2.28 min

LCMS method G: [M+H]⁺=327.3, t_{R}=2.24 min

¹H NMR (400 MHz, DMSO) 12.67 (1H, s), 8.57 (1H, s), 7.66-7.65 (1H, m), 7.39-7.36 (2H, m), 6.92 (1H, dd, J=2.3, 8.9 Hz), 4.49-4.39 (2H, m), 4.32-4.24 (1H, m), 3.75-3.54 (3H, m), 2.51-2.42 (1H, m), 2.22-2.14 (1H, m), 2.04-1.95 (1H, m), 1.42-1.39 (4H, m), 1.22-1.20 (3H, m) ppm.

### Example 98: (8R,12R)-8,12-dimethyl-9,13-dioxa-4,5,18,19-tetraazatetracyclo[12.5.2.1^{2,5}.0^{17,20}]docosa-1(19),2(22),3,14(21),15,17(20)-hexaene

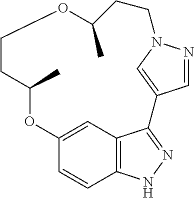 [SMILES: C[C@@H]1CCn2cc(cn2)-c2n[nH]c3ccc(cc23)O[C@H](C)CCO1]

Example 98 is prepared according to the synthesis route described in general Scheme E following the same synthetic procedures as example 97, starting with (R)-methyl 3-hydroxybutanoate. To a solution of (8R,12R)-8,12-dimethyl-18-(oxan-2-yl)-9,13-dioxa-4,5,18,19-tetraazatetra cyclo[12.5.2.1^{2,5}.0^{17,20}]docosa-1(19),2(22),3,14(21),15,17(20)-hexaene (65 mg, 0.16 mmol) in methanol (7 mL) and water (1 mL) was added p-toluenesulfonic acid monohydrate (151 mg, 0.79 mmol). The reaction mixture was stirred at 65° C. overnight. The reaction mixture was concentrated under reduced pressure and the residue was neutralized by slow of saturated aqueous NaHCO₃ solution. The residue was diluted with ethyl acetate. The phases were separated and the aqueous phase was extracted with ethyl acetate. The combined organic layers were washed with brine, dried over sodium sulfate, filtered and evaporated under reduced pressure. The residue was purified by silica gel chromatography using dichloro methane/methanol 100/0 to 94/6 as eluent. The resulting oil was crystallized with acetonitrile, filtered and dried to afford (8R,12R)-8,12-dimethyl-9,13-dioxa-4,5,18,19-tetraazatetracyclo [12.5.2.1^{2,5}.0^{17,20}]docosa-1(19),2(22),3,14(21),15,17(20)-hexaene as a solid.

LCMS method F: [M+H]⁺=327.3, t_{R}=2.29 min

LCMS method G: [M+H]⁺=327.3, t_{R}=2.25 min

¹H NMR (400 MHz, DMSO) 12.66-12.64 (1H, m), 8.66 (1H, s), 7.67-7.66 (1H, m), 7.46 (1H, d, J=2.3 Hz), 7.40-7.37 (1H, m), 6.92 (1H, dd, J=2.5, 8.9 Hz), 4.57-4.41 (2H, m), 4.31-4.24 (1H, m), 3.87-3.83 (2H, m), 3.39-3.28 (1H, m), 2.38-2.21 (3H, m), 1.56-1.48 (1H, m), 1.41 (3H, d, J=6.1 Hz), 1.23-1.21 (3H, m) ppm.

### Example 99: (12S,13R)-12,13-dimethyl-8,11,14-trioxa-4,5,19,20-tetraazatetracyclo [13.5.2.1^{2,5}.0^{18,21}]tricosa-1(20),2(23),3,15(22),16,18(21)-hexaene

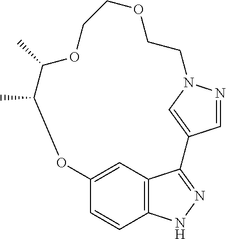 [SMILES: C[C@@H]1OCCOCCn2cc(cn2)-c2n[nH]c3ccc(cc23)O[C@@H]1C]

Example 99 is prepared according to the synthesis route described in general Scheme D.

### Preparation of Intermediate 594: (2S,3S)-3-(benzyloxy)butan-2-ol

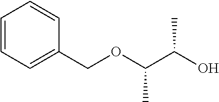

To a solution of (2S,3S)-butane-2,3-diol (510 mg, 5.66 mmol) in DMF (25 mL) at 0° C. was added portionwise sodium hydride (60% dispersion in mineral oil) (226 mg, 5.66 mmol) and the reaction mixture was stirred for 20 min. Then benzyl bromide (404 μL, 3.40 mmol) was added dropwise and the suspension was stirred at RT for 16 h. Water was added and the aqueous phase was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 75/25 as eluent to afford (2S,3S)-3-(benzyloxy)butan-2-ol as a colorless oil.

LCMS method F: [M+H]⁺=not detected, t_{R}=2.01 min

### Preparation of Intermediate 595: 2-[2-(4-bromo-1H-pyrazol-1-yl)ethoxy]ethyl

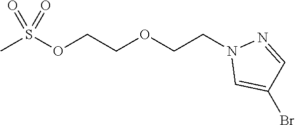 [SMILES: CS(=O)(=O)OCCOCCn1cc(Br)cn1]

To a suspension 2-[2-(4-bromo-1H-pyrazol-1-yl)ethoxy]ethan-1-ol (1.262 g, 5.37 mmol) in dichloromethane (27 mL) at 0° C. was added triethylamine (1.12 mL, 8.05 mmol) and methanesulfonyl chloride (496 μL, 6.44 mmol). The reaction mixture was stirred at RT for 1 h. The reaction mixture was diluted with water, the layers were separated and the aqueous layer was extracted with dichloromethane. The combined organic layers were washed with saturated aqueous ammonium chloride solution, water and brine, dried over anhydrous sodium sulfate, filtered and evaporated under reduced pressure to afford 2-[2-(4-bromo-1H-pyrazol-1-yl) ethoxy]ethylmethanesulfonate as a light yellow oil which was used without further purification LCMS method F: [M+H]⁺=313.0-315.0, t_{R}=1.89 min

### Preparation of Intermediate 596: 1-[2-[2-[(1S,2S)-2-benzyloxy-1-methyl-propoxy] ethoxy]ethyl]-4-bromo-pyrazole

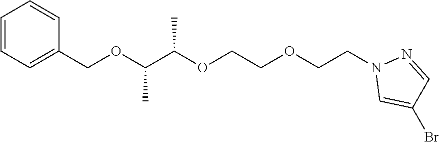 [SMILES: C[C@H](OCCOCCn1cc(Br)cn1)[C@H](C)OCc1ccccc1]

To a solution of (2S,3S)-3-(benzyloxy)butan-2-ol (261 mg, 1.45 mmol) in DMF (4.25 mL) at 0° C. was added sodium hydride (60% dispersion in mineral oil) (87 mg, 2.17 mmol) and 2-[2-(4-bromo-1H-pyrazol-1-yl)ethoxy]ethyl methanesulfonate (680 mg, 2.17 mmol) in DMF (3 mL). The reaction mixture was stirred at RT for 48 h. The reaction mixture was diluted with ethyl acetate, washed with water then brine. The organic layers were dried over anhydrous sodium sulfate, filtered and dried under reduced pressure. The residue was purified by silica gel chromatography using cyclohexane/ethyl acetate 70/30 as eluent to afford 1-[2-[2-[(1S,2S)-2-benzyloxy-1-methyl-propoxy]ethoxy]ethyl]-4-bromo-pyrazole as a light yellow oil.

LCMS method F: [M+H]⁺=397.1-399.1, t_{R}=2.88 min

### Preparation of Intermediate 597: [3-[1-[2-[2-[(1S,2S)-2-benzyloxy-1-methyl-propoxy]ethoxy]ethyl]pyrazol-4-yl]-1-tetrahydropyran-2-yl-indazol-5-yl]oxy-tert-butyl-dimethyl-silane

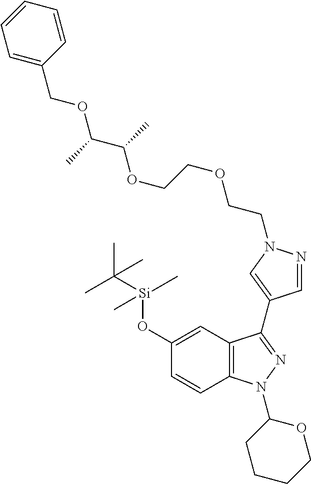 [SMILES: C[C@H](OCCOCCn1cc(-c2nn(C3CCCCO3)c3ccc(O[Si](C)(C)C(C)(C)C)cc23)cn1)[C@H](C)OCc1ccccc1]

To a suspension of 1-[2-[2-[(1S,2S)-2-benzyloxy-1-methyl-propoxy]ethoxy]ethyl]-4-bromo-pyrazole (428 mg, 1.08 mmol) in dioxane (4.5 mL) and water (0.5 mL) was added tert-butyl-dimethyl-[1-tetrahydropyran-2-yl-3-(4,4,5,5-tetramethyl-1,3,2-dioxaborolan-2-yl)indazol-5-yl]oxy-silane (642 mg, 1.40 mmol), and potassium phosphate tribasic (686 mg, 3.23 mmol). The reaction mixture was purged with argon for 15 min then tetrakis(triphenylphosphine) palladium(0) (62 mg, 0.05 mmol) and Xphos (51 mg, 0.11 mmol) were added. The reaction mixture was heated under microwave irradiations at 90° C. for 1.5 h. The reaction mixture was poured into water then extracted with ethyl acetate. The organic layers were washed with brine, dried over anhydrous sodium sulfate, filtered and evaporated under reduced pressure. The residue was purified by silica gel chromatography using cyclohexane/ethyl acetate 70/30 as eluent to afford [3-[1-[2-[2-[(1S,2S)-2-benzyloxy-1-methyl-propoxy]ethoxy]ethyl]pyrazol-4-yl]-1-tetrahydropyran-2-yl-indazol-5-yl]oxy-tert-butyl-dimethyl-silane as a brown oil.

LCMS method F: [M+H]⁺=649.4, t_{R}=3.77 min

### Preparation of Intermediate 598: (2S,3S)-3-[2-[2-[4-[5-[tert-butyl(dimethyl)silyl]oxy-1-tetrahydropyran-2-yl-indazol-3-yl]pyrazol-1-yl]ethoxy]ethoxy]butan-2-ol

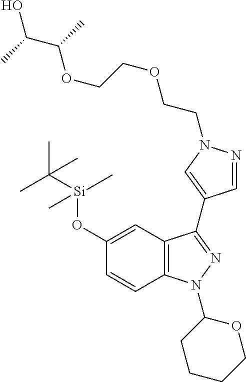 [SMILES: C[C@H](O)[C@H](C)OCCOCCn1cc(-c2nn(C3CCCCO3)c3ccc(O[Si](C)(C)C(C)(C)C)cc23)cn1]

To a solution of [3-[1-[2-[2-[(1S,2S)-2-benzyloxy-1-methyl-propoxy]ethoxy]ethyl]pyrazol-4-yl]-1-tetrahydropyran-2-yl-indazol-5-yl]oxy-tert-butyl-dimethyl-silane (430 mg, 0.66 mmol) in ethanol (5 mL) under argon was added palladium on carbon 10% wt (43 mg, 0.40 mmol). The reaction mixture was stirred under hydrogen atmosphere at RT for 16 h. The reaction mixture was filtered and the solvent was evaporated under reduced pressure to afford (2S,3S)-3-[2-[2-[4-[5-[tert-butyl(dimethyl)silyl]oxy-1-tetrahydropyran-2-yl-indazol-3-yl]pyrazol-1-yl]ethoxy] ethoxy]butan-2-ol as a light yellow oil.

LCMS method F: [M+H]⁺=559.5, t_{R}=3.32 min

### Preparation of Intermediate 599: [(1S,2S)-2-[2-[2-[4-[5-[tert-butyl(dimethyl)silyl] oxy-1-tetrahydropyran-2-yl-indazol-3-yl]pyrazol-1-yl]ethoxy]ethoxy]-1-methyl-propyl]methanesulfonate

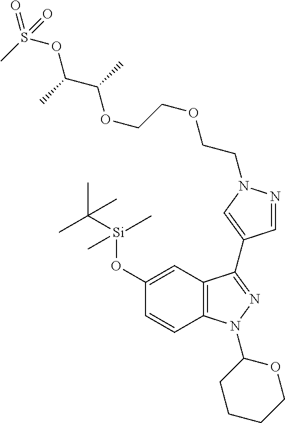 [SMILES: C[C@H](OCCOCCn1cc(-c2nn(C3CCCCO3)c3ccc(O[Si](C)(C)C(C)(C)C)cc23)cn1)[C@H](C)OS(C)(=O)=O]

To a suspension (2S,3S)-3-[2-[2-[4-[5-[tert-butyl(dimethyl)silyl]oxy-1-tetrahydropyran-2-yl-indazol-3-yl]pyrazol-1-yl]ethoxy]ethoxy]butan-2-ol (405 mg, 0.72 mmol) in dichloromethane (4 mL) at 0° C. was added triethylamine (151 μL, 1.09 mmol) and methanesulfonyl chloride (67 μL, 0.87 mmol). The reaction mixture was stirred at RT for 1 h. The reaction mixture was diluted with water. The layers were separated and the aqueous layer was extracted with dichloro methane. The combined organic layers were washed with brine, dried over anhydrous sodium sulfate, filtered and evaporated under reduced pressure to afford [(1S,2S)-2-[2-[2-[4-[5-[tert-butyl(dimethyl)silyl]oxy-1-tetrahydropyran-2-yl-indazol-3-yl]pyrazol-1-yl]ethoxy]ethoxy]-1-methyl-propyl]methanesulfonate as a light yellow oil which was used in the next step without further purification.

LCMS method F: [M+H]⁺=637.5, t_{R}=3.42 min

### Preparation of Intermediate 600: (12S,13R)-12,13-dimethyl-19-(oxan-2-yl)-8,11,14-trioxa-4,5,19,20-tetraazatetracyclo[13.5.2.1^{2,5}.0^{18,21}]tricosa-1(20),2(23),3,15(22),16, 18(21)-hexaene

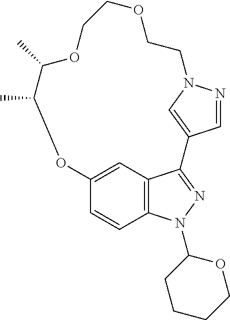 [SMILES: C[C@@H]1OCCOCCn2cc(cn2)-c2nn(C3CCCCO3)c3ccc(cc23)O[C@@H]1C]

To a suspension of cesium carbonate (737 mg, 2.26 mmol) in anhydrous DMF (20 mL) at 80° C. was added dropwise [(1S,2S)-2-[2-[2-[4-[5-[tert-butyl(dimethyl)silyl]oxy-1-tetrahydropyran-2-yl-indazol-3-yl]pyrazol-1-yl]ethoxy]ethoxy]-1-methyl-propyl]methanesulfonate (480 mg, 0.75 mmol) in DMF (10 mL). The reaction mixture was stirred at 80° C. for 30 min. The reaction mixture was filtered and concentrated under pressure. The residue was purified by silica gel column chromatography using cyclohexane/ethyl acetate 50/50 as eluent to afford (12S,13R)-12,13-dimethyl-19-(oxan-2-yl)-8,11,14-trioxa-4,5,19,20-tetraazatetracyclo[13.5.2.1^{2,5}.0^{18,21}] tricosa-1(20),2(23),3,15(22),16,18(21)-hexaene as a yellow oil.

LCMS method F: [M+H]⁺=427.5, t_{R}=2.69 min

### Preparation of Example 99: (12S,13R)-12,13-dimethyl-8,11,14-trioxa-4,5,19,20-tetraazatetracyclo[13.5.2.1^{2,5}.0^{18,21}]tricosa-1(20),2(23),3,15(22),16,18(21)-hexaene

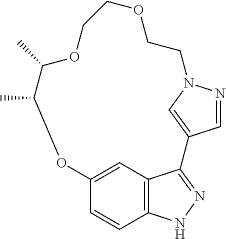 [SMILES: C[C@@H]1OCCOCCn2cc(cn2)-c2n[nH]c3ccc(cc23)O[C@@H]1C]

To a solution of ((12S,13R)-12,13-dimethyl-19-(oxan-2-yl)-8,11,14-trioxa-4,5,19,20-tetraaza tetracyclo[13.5.2.1^{2,5}.0^{18,21}]tricosa-1(20),2(23),3,15(22),16,18(21)-hexaene (18 mg, 0.04 mmol) in methanol (1.3 mL) and water (0.2 mL) was added p-toluenesulfonic acid monohydrate (40 mg, 0.21 mmol). The reaction mixture was stirred at 80° C. for 16 h. The reaction mixture was dissolved in ethyl acetate then a saturated aqueous NaHCO₃ solution was added. The phases were separated and the aqueous layer was extracted with ethyl acetate. The combined organic layers were washed with brine, dried over sodium sulfate, filtered and evaporated under reduced pressure. The resulting solid was recrystallized with acetonitrile, filtered and dried to afford (12S,13R)-12,13-dimethyl-8,11,14-trioxa-4,5,19,20-tetraazatetracyclo[13.5.2.1^{2,5}.0^{18,21}] tricosa-1(20),2(23),3,15(22),16,18(21)-hexaene as a solid.

LCMS method F: [M+H]⁺=343.3, t_{R}=2.10 min

LCMS method G: [M+H]⁺=343.3, t_{R}=2.11 min

1H NMR (400 MHz, CDCl₃) 8.56 (1H, s), 8.11-8.00 (3H, m), 7.32 (1H, d, J=9.0 Hz), 7.12-7.09 (1H, m), 4.73-4.68 (1H, m), 4.61-4.54 (1H, m), 4.50-4.45 (2H, m), 3.99-3.56 (6H, m), 1.36 (3H, d, J=6.6 Hz), 1.28 (3H, d, J=6.7 Hz) ppm.

### Example 100: (12R,13S)-12,13-dimethyl-8,11,14-trioxa-4,5,19,20-tetraazatetra cyclo [13.5.2.1^{2,5}.0^{18,21}]tricosa-1(20),2(23),3,15(22),16,18(21)-hexaene

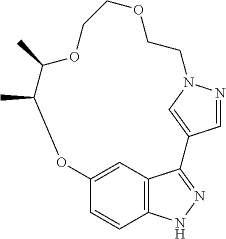 [SMILES: C[C@@H]1Oc2ccc3[nH]nc(c3c2)-c2cnn(c2)CCOCCO[C@@H]1C]

Example 100 is prepared according to the synthesis route described in general Scheme D following the same procedures as Example 99 but starting from (2R,3R)-3-(benzyloxy)butan-2-ol.

### Preparation of Intermediate 601: (12R,13S)-12,13-dimethyl-19-(oxan-2-yl)-8,11,14-trioxa-4,5,19,20-tetraazatetracyclo[13.5.2.1^{2,5}.0^{18,21}]tricosa-1(20),2(23),3,15(22), 16,18(21)-hexaene

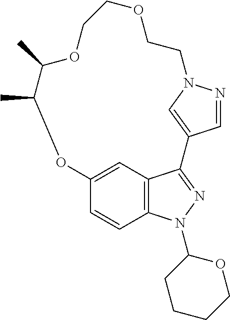 [SMILES: C[C@@H]1Oc2ccc3c(c2)c(nn3C2CCCCO2)-c2cnn(c2)CCOCCO[C@@H]1C]

To a suspension of cesium carbonate (579 mg, 1.78 mmol) in anhydrous DMF (40 mL) at 80° C. was added dropwise [(1R,2R)-2-[2-[2-[4-[5-[tert-butyl(dimethyl)silyl]oxy-1-tetrahydropyran-2-yl-indazol-3-yl]pyrazol-1-yl]ethoxy]ethoxy]-1-methyl-propyl]methanesulfonate (377 mg, 0.59 mmol) in DMF (30 mL). The reaction mixture was stirred at 80° C. for 30 min. The reaction mixture was filtered and concentrated under reduced pressure. The residue was purified by silica column chromatography using cyclohexane/ethyl acetate 50/50 as eluent to afford (12R,13S)-12,13-dimethyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene as a yellow oil.

LCMS method F: [M+H]⁺=427.4, t_{R}=2.69 min

### Preparation of Example 100: (12R,13S)-12,13-dimethyl-8,11,14-trioxa-4,5,19,20-tetraazatetra cyclo[13.5.2.1^{2,5}.0^{18,21}]tricosa-1(20),2(23),3,15(22),16,18(21)-hexaene

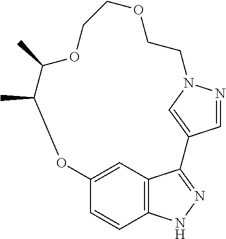 [SMILES: C[C@@H]1Oc2ccc3[nH]nc(c3c2)-c2cnn(c2)CCOCCO[C@@H]1C]

To a solution of (12R,13S)-12,13-dimethyl-19-(oxan-2-yl)-8,11,14-trioxa-4,5,19,20-tetraaza tetracyclo[13.5.2.1^{2,5}.0^{18,21}]tricosa-1(20),2(23),3,15(22),16,18(21)-hexaene (10 g, 0.02 mmol) in methanol (1.3 mL) and water (0.2 mL) was added p-toluenesulfonic acid monohydrate (22 mg, 0.12 mmol) and the reaction mixture was stirred at 80° C. for 16 h. The reaction mixture was dissolved in ethyl acetate and a saturated aqueous NaHCO₃ solution was added. The phases were separated and the aqueous layer was extracted with ethyl acetate. The combined organic layers were was washed with brine, dried over anhydrous sodium sulfate, filtered and evaporated under reduced pressure. The resulting solid was then recrystallized with acetonitrile, filtered and dried to afford (12R,13S)-12,13-dimethyl-8,11,14-trioxa-4,5,19,20-tetraazatetra cyclo[13.5.2.1^{2,5}.0^{18,21}]tricosa-1(20),2(23),3,15(22),16,18(21)-hexaene as a solid.

LCMS method F: [M+H]⁺=343.3, t_{R}=2.10 min

LCMS method G: [M+H]⁺=343.3, t_{R}=2.09 min

1H NMR (400 MHz, CDCl₃) 8.58 (1H, s), 8.12-8.01 (3H, m), 7.32 (1H, d, J=8.7 Hz), 7.11 (1H, dd, J=2.3, 8.9 Hz), 4.71 (1H, m), 4.6 (1H, m), 4.48 (2H, dd, m), 3.78-3.56 (6H, m), 1.39-1.35 (3H, m), 1.30-1.26 (3H, m) ppm.

### Example 101: (6S,13S)-6,13-dimethyl-8,11,14-trioxa-4,5,19,20-tetraazatetracyclo[13.5.2.1^{2,5}.0^{18,21}]tricosa-1(20),2(23),3,15(22),16,18(21)-hexaene

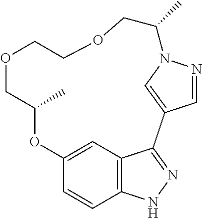 [SMILES: C[C@H]1COCCOC[C@H](C)n2cc(cn2)-c2n[nH]c3ccc(cc23)O1]

Example 101 is prepared according to the synthesis route described in general Scheme D.

### Preparation of Intermediate 602: (2R)-1-trityloxypropan-2-ol

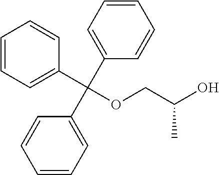 [SMILES: C[C@@H](O)COC(c1ccccc1)(c1ccccc1)c1ccccc1]

To a solution of (2R)-propane-1,2-diol (962 μL, 13.14 mmol) in dichloromethane (30 mL) at 0° C. was added triethylamine (2.381 mL, 17.09 mmol) followed by dropwise addition of trityl chloride (3.664 g, 13.14 mmol) in dichloromethane (10 mL). The reaction mixture was stirred at RT overnight. Water was added to the suspension and the phases were separated. The aqueous layer was extracted with dichloromethane and the combined organic layers were washed with brine, dried over anhydrous sodium sulfate, filtered and evaporated under reduced pressure to afford (2R)-1-trityloxypropan-2-ol was obtained as a colorless oil.

LCMS method F: [M+Na]⁺=341.2, t_{R}=3.01 min

### Preparation of Intermediate 603: [(1R)-1-methyl-2-trityloxy-ethyl]methanesulfonate

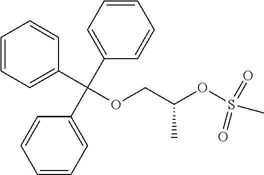 [SMILES: C[C@H](COC(c1ccccc1)(c1ccccc1)c1ccccc1)OS(C)(=O)=O]

To a solution of (2R)-1-trityloxypropan-2-ol (1 g, 3.14 mmol) in dichloromethane (15 mL) at 0° C. was added triethylamine (869 μL, 6.28 mmol) and methanesulfonyl chloride (310 μL, 4.09 mmol). The reaction mixture was stirred at RT for 1.5 h. Water and dichloromethane were added and the phases were separated. The organic layer was washed with a saturated aqueous NaHCO₃ solution then with saturated aqueous ammonium chloride solution. The organic layer was dried over anhydrous sodium sulfate, filtered and evaporated under reduced pressure to afford [(1R)-1-methyl-2-trityloxy-ethyl] methanesulfonate as a yellow oil which as in the next step without further purification.

LCMS method F: [M+H]⁺=not detected, t_{R}=3.13 min

### Preparation of Intermediate 604: 4-bromo-1-[(1S)-1-methyl-2-trityloxy-ethyl] pyrazole

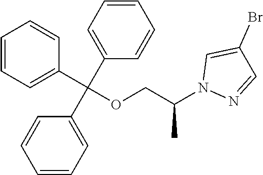 [SMILES: C[C@@H](COC(c1ccccc1)(c1ccccc1)c1ccccc1)n1cc(Br)cn1]

To a solution of 4-bromo-1H-pyrazole (438 mg, 2.98 mmol) in acetonitrile (20 mL) was added cesium carbonate (1.26 g, 3.87 mmol) and [(1R)-1-methyl-2-trityloxy-ethyl] methanesulfonate (1.42 g, 3.58 mmol). The reaction mixture was stirred at 85° C. for 4 h. The reaction mixture was cooled to RT and water and ethyl acetate were added. The layers were separated and the aqueous layer was extracted with ethyl acetate. The combined organic layers were dried over anhydrous sodium sulfate, filtered and evaporated under reduced pressure to afford 4-bromo-1-[(1S)-1-methyl-2-trityloxy-ethyl]pyrazole as a colorless oil which was used in the next step without further purification.

LCMS method F: [M+H]⁺=not detected, t_{R}=3.45 min

### Preparation of Intermediate 605: (2S)-2-(4-bromopyrazol-1-yl)propan-1-ol

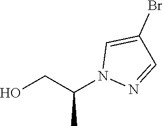

To a solution of 4-bromo-1-[(1S)-1-methyl-2-trityloxy-ethyl]pyrazole (1.43 g, 3.21 mmol) in methanol (2 mL) was added p-toluenesulfonic acid (61 mg, 0.32 mmol). The reaction mixture was stirred at RT overnight. The solvent was evaporated under reduced pressure. The residue was purified by silica gel column chromatography using cyclohexane/ethyl acetate 100/0 to 70/30 as eluent to afford (2S)-2-(4-bromopyrazol-1-yl)propan-1-ol as a colorless oil.

LCMS method F: [M+H]⁺=205.1-207.1, t_{R}=1.48 min

### Preparation of Intermediate 606: 1-[(1S)-2-[2-[(2R)-2-benzyloxypropoxy]ethoxy]-1-methyl-ethyl]-4-bromo-pyrazole

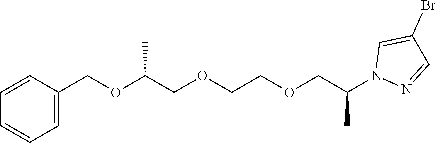 [SMILES: C[C@H](COCCOC[C@H](C)n1cc(Br)cn1)OCc1ccccc1]

To a solution of (2S)-2-(4-bromopyrazol-1-yl)propan-1-ol (115 mg, 0.56 mmol) in dry DMF (3 mL) was added sodium hydride (60% dispersion in mineral oil) (34 mg, 0.84 mmol). The reaction mixture was stirred at RT for 10 min then a solution of 2-[(2R)-2-(benzyloxy) propoxy]ethylmethanesulfonate (intermediate 387) (210 mg, 0.73 mmol) in dry DMF (2 mL) was added. The reaction mixture was stirred at RT for 4 h. The reaction was quenched with water and ethyl acetate was added. The phases were separated and the aqueous layer was extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 70/30 as eluent to afford 1-[(1S)-2-[2-[(2R)-2-benzyloxy propoxy]ethoxy]-1-methyl-ethyl]-4-bromo-pyrazole as a colorless oil.

LCMS method J: [M+H]⁺=397.2-399.2, t_{R}=4.19 min

### Preparation of Intermediate 607: [3-[1-[(1S)-2-[2-[(2R)-2-benzyloxypropoxy] ethoxy]-1-methyl-ethyl]pyrazol-4-yl]-1-tetrahydropyran-2-yl-indazol-5-yl]oxy-tert-butyl-dimethyl-silane

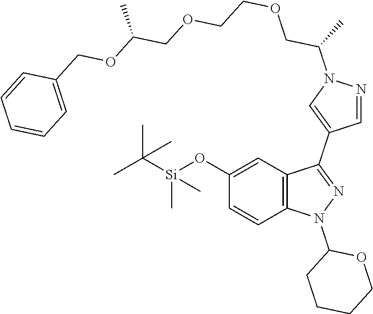 [SMILES: C[C@H](COCCOC[C@H](C)n1cc(-c2nn(C3CCCCO3)c3ccc(O[Si](C)(C)C(C)(C)C)cc23)cn1)OCc1ccccc1]

To a degassed solution of 1-[(1S)-2-[2-[(2R)-2-benzyloxypropoxy]ethoxy]-1-methyl-ethyl]-4-bromo-pyrazole (159 mg, 0.40 mmol), tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (220 mg, 0.48 mmol) and potassium phosphate tribasic (254 mg, 1.20 mmol) in dioxane (3.5 mL) and water (0.15 mL) was added tetrakis(triphenylphosphine)palladium(0) (23 mg, 0.02 mmol) and 2-dicyclohexylphosphino-2′,4′,6′-triisopropylbiphenyl (19 mg, 0.04 mmol). The reaction mixture was stirred at 135° C. for 1 h. The reaction mixture was filtered through a pad of Celite and washed with ethyl acetate. The filtrate was diluted with water and extracted with ethyl acetate. The combined organic layers were washed with water and brine, dried over anhydrous magnesium sulfate, filtered and evaporated under reduced pressure. The residue was purified by silica gel column chromatography using cyclohexane/ethyl acetate 100/0 to 70/30 as eluent to afford [3-[1-[(1S)-2-[2-[(2R)-2-benzyloxypropoxy]ethoxy]-1-methyl-ethyl]pyrazol-4-yl]-1-tetrahydropyran-2-yl-indazol-5-yl]oxy-tert-butyl-dimethyl-silane as a colorless oil.

LCMS method J: [M+H]⁺=649.4, t_{R}=5.92 min

### Preparation of Intermediate 608: (2R)-1-[2-[(2S)-2-[4-[5-[tert-butyl(dimethyl)silyl] oxy-1-tetrahydropyran-2-yl-indazol-3-yl]pyrazol-1-yl]propoxy]ethoxy]propan-2-ol

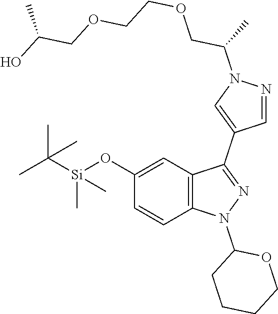 [SMILES: C[C@@H](O)COCCOC[C@H](C)n1cc(-c2nn(C3CCCCO3)c3ccc(O[Si](C)(C)C(C)(C)C)cc23)cn1]

To a solution of [3-[1-[(1S)-2-[2-[(2R)-2-benzyloxypropoxy]ethoxy]-1-methyl-ethyl]pyrazol-4-yl]-1-tetrahydropyran-2-yl-indazol-5-yl]oxy-tert-butyl-dimethyl-silane (200 mg, 0.31 mmol) in ethanol (4 mL) at RT was added palladium on carbon 10 wt. % loading (10 mg). The reaction mixture was stirred under hydrogen atmosphere at RT overnight. The reaction mixture was filtered and the filtrate was concentrated under reduced pressure. The residue was purified by silica gel column chromatography using cyclohexane/ethyl acetate 100/0 to 50/50 as eluent to afford (2R)-1-[2-[(2S)-2-[4-[5-[tert-butyl(dimethyl)silyl]oxy-1-tetrahydropyran-2-yl-indazol-3-yl]pyrazol-1-yl]propoxy]ethoxy]propan-2-ol as a colorless oil.

LCMS method F: [M+H]⁺=559.3, t_{R}=3.28 min

### Preparation of Intermediate 609: [(1R)-2-[2-[(2S)-2-[4-[5-[tert-butyl(dimethyl)silyl] oxy-1-tetrahydropyran-2-yl-indazol-3-yl]pyrazol-1-yl]propoxy]ethoxy]-1-methyl-ethyl] methanesulfonate

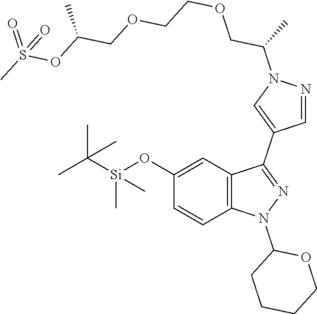 [SMILES: C[C@H](COCCOC[C@H](C)n1cc(-c2nn(C3CCCCO3)c3ccc(O[Si](C)(C)C(C)(C)C)cc23)cn1)OS(C)(=O)=O]

To a solution of (2R)-1-[2-[(2S)-2-[4-[5-[tert-butyl(dimethyl)silyl]oxy-1-tetrahydropyran-2-yl-indazol-3-yl]pyrazol-1-yl]propoxy]ethoxy]propan-2-ol (67 mg, 0.12 mmol) in dichloro methane (1 mL) at 0° C. was added triethylamine (33 μL, 0.24 mmol) and methanesulfonyl chloride (12 μL, 0.15 mmol). The reaction mixture was stirred at 0° C. for 10 min then warmed to RT and stirred for 12 h. The reaction mixture was quenched with a saturated aqueous ammonium chloride solution then extracted with dichloromethane. The combined organic layers were washed with brine, dried over anhydrous magnesium sulfate, filtered and evaporated under reduced pressure to afford [(1R)-2-[2-[(2S)-2-[4-[5-[tert-butyl(dimethyl) silyl]oxy-1-tetrahydropyran-2-yl-indazol-3-yl]pyrazol-1-yl]propoxy]ethoxy]-1-methyl-ethyl] methanesulfonate as a colorless oil which was used in the next step without further purification.

LCMS method F: [M+H]⁺=637.3, t_{R}=3.42 min

### Preparation of Intermediate 610: (6S,13S)-6,13-dimethyl-19-(oxan-2-yl)-8,11,14-trioxa-4,5,19,20-tetraazatetracyclo[13.5.2.1^{2,5}.0^{18,21}]tricosa-1(20),2(23),3,15(22),16, 18(21)-hexaene

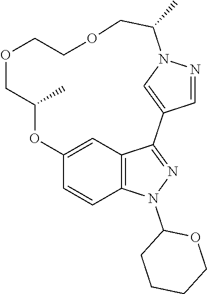 [SMILES: C[C@H]1COCCOC[C@H](C)n2cc(cn2)-c2nn(C3CCCCO3)c3ccc(cc23)O1]

To a suspension of cesium carbonate (84 mg, 0.258 mmol) in anhydrous DMF (4 mL) at 65° C. was added dropwise [(1R)-2-[2-[(2S)-2-[4-[5-[tert-butyl(dimethyl)silyl]oxy-1-tetrahydro pyran-2-yl-indazol-3-yl]pyrazol-1-yl]propoxy]ethoxy]-1-methyl-ethyl]methanesulfonate (55 mg, 0.086 mmol) in DMF (2 mL). The resulting suspension was stirred at 65° C. for 8 h. The reaction mixture was cooled down to RT, filtered over a pad of Celite and washed with ethyl acetate. The filtrate was concentrated under reduced pressure. The residue was purified by preparative TLC eluting with diisopropylether/ethyl acetate/ethanol 6/3/1 to afford (6S,13S)-6,13-dimethyl-19-(oxan-2-yl)-8,11,14-trioxa-4,5,19,20-tetraazatetracyclo[13.5.2.1^{2,5}.0^{18,21}] tricosa-1(20),2(23),3,15(22),16,18(21)-hexaene as a white solid.

LCMS method F: [M+H]⁺=427.2, t_{R}=2.74 min

### Preparation of Example 101: (6S,13S)-6,13-dimethyl-8,11,14-trioxa-4,5,19,20-tetraazatetracyclo[13.5.2.1^{2,5}.0^{18,21}]tricosa-1(20),2(23),3,15(22),16,18(21)-hexaene

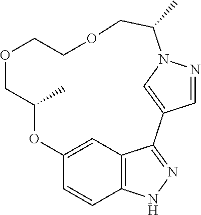 [SMILES: C[C@H]1COCCOC[C@H](C)n2cc(cn2)-c2n[nH]c3ccc(cc23)O1]

To a solution of (6S,13 S)-6,13-dimethyl-19-(oxan-2-yl)-8,11,14-trioxa-4,5,19,20-tetraazatetra cyclo[13.5.2.1^{2,5}.0^{18,21}]tricosa-1(20),2(23),3,15(22),16,18(21)-hexaene (19 mg, 0.045 mmol) in methanol (700 μL) and water (100 μL) was added p-toluenesulfonic acid monohydrate (41 mg, 0.22 mmol). The reaction mixture was stirred at 65° C. for 6 h. The reaction mixture was diluted with dichloromethane and saturated aqueous NaHCO₃ solution. The phases were separated and the aqueous layer was extracted with dichloromethane. The combined organic layers were washed brine, dried over anhydrous magnesium sulfate, filtered and evaporated under reduced pressure. The residue was purified by preparative TLC eluting with dichloromethane/methanol 95/5 to afford (6S,13S)-6,13-dimethyl-8,11,14-trioxa-4,5,19,20-tetraazatetracyclo[13.5.2.1^{2,5}.0^{18,21}]tricosa-1(20),2(23),3,15(22),16,18(21)-hexaene as a solid.

LCMS method F: [M+H]⁺=343.3, t_{R}=2.16 min

LCMS method G: [M+H]⁺=343.3, t_{R}=2.12 min

¹H NMR (400 MHz, CDCl₃) 8.61 (1H, d, J=0.8 Hz), 8.03 (1H, d, J=2.4 Hz), 8.02 (1H, d, J=0.8 Hz), 7.33 (1H, dd, J=8.8, 0.8 Hz), 7.09 (1H, dd, J=8.8, 2.4 Hz), 4.71-4.63 (1H, m), 4.55-4.47 (1H, m), 3.92-3.55 (8H, m), 1.70 (3H, d, J=7.2 Hz), 1.44 (3H, d, J=6.8 Hz) ppm.

### Example 102: 8,13-dioxa-4,5,18,19-tetraazatetracyclo[12.5.2.1^{2,5}.0^{17,20}]docosa-1(19), 2(22),3,14(21),15,17(20)-hexaene

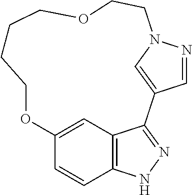 [SMILES: c1cc2[nH]nc3c2cc1OCCCCOCCn1cc-3cn1]

Example 102 is prepared according to the synthesis route described in general scheme D.

### Preparation of Intermediate 611: 2-[4-(benzyloxy)butoxy]ethylmethanesulfonate

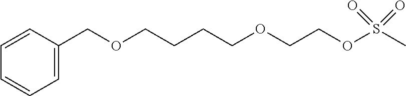 [SMILES: CS(=O)(=O)OCCOCCCCOCc1ccccc1]

To a solution of 2-[4-(benzyloxy)butoxy]ethan-1-ol (970 mg, 4.32 mmol) in dichloromethane (15 mL) at 0° C. was added triethylamine (1.2 mL, 8.64 mmol) then methanesulfonyl chloride (0.435 mL, 5.62 mmol). The reaction mixture was stirred at RT for 1 h. Water was added, the phases were separated and the organic layer was dried over anhydrous sodium sulfate, filtered and concentrated under reduced pressure. The residue was purified by silica gel column chromatography using cyclohexane/ethyl acetate 100/0 to 60/40 as eluent to afford 2-[4-(benzyloxy)butoxy]ethylmethanesulfonate as a colorless oil.

¹H NMR (400 MHz, CDCl₃) 7.37-7.35 (5H, m), 4.52 (2H, s), 4.39-4.36 (2H, m), 3.72-3.69 (2H, m), 3.54-3.50 (4H, m), 3.06 (3H, s), 1.72-1.69 (4H, m) ppm.

### Preparation of Intermediate 612: 1-{2-[4-(benzyloxy)butoxy]ethyl}-4-bromo-1H-pyrazole

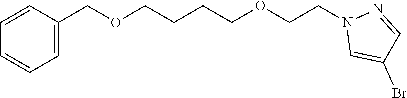 [SMILES: Brc1cnn(CCOCCCCOCc2ccccc2)c1]

To a solution of 2-[4-(benzyloxy)butoxy]ethylmethanesulfonate (1.21 g, 4 mmol) in acetonitrile (40 mL) was added 4-bromo-1H-pyrazole (647 mg, 4.4 mmol). The reaction mixture was stirred at 80° C. for 2 h then at 50° C. overnight. The reaction mixture was filtered and the solvent was evaporated under vacuo. The residue was purified by silica gel column chromatography using cyclohexane/ethyl acetate 100/0 to 60/40 as eluent to afford 1-{2-[4-(benzyloxy)butoxy]ethyl}-4-bromo-1H-pyrazole as a colorless oil.

LCMS method F: [M+H]⁺=353.2-355.2, t_{R}=2.86 min

### Preparation of Intermediate 613: 3-(1-{2-[4-(benzyloxy)butoxy]ethyl}-1H-pyrazol-4-yl)-5-[(tert-butyldimethylsilyl) oxy]-1-(oxan-2-yl)-1H-indazole

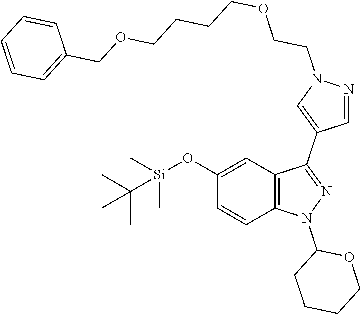 [SMILES: CC(C)(C)[Si](C)(C)Oc1ccc2c(c1)c(-c1cnn(CCOCCCCOCc3ccccc3)c1)nn2C1CCCCO1]

To a degassed solution of 1-{2-[4-(benzyloxy)butoxy]ethyl}-4-bromo-1H-pyrazole (780 mg, 2.21 mmol), tert-butyl-dimethyl-[1-tetrahydropyran-2-yl-3-(4,4,5,5-tetramethyl-1,3,2-dioxa borolan-2-yl)indazol-5-yl]oxy-silane (intermediate 61) (1.21 g, 2.65 mmol) and potassium phosphate tribasic (1.40 g, 6.63 mmol) in dioxane (4 mL) and water (0.2 mL) was added tetrakis(triphenylphosphine)palladium(0) (128 mg, 0.111 mmol) and 2-dicyclohexyl phosphino-2′,4′,6′-triisopropylbiphenyl (105 mg, 0.221 mmol). The reaction mixture was stirred at 90° C. for 2 h. The reaction mixture was cooled to RT, diluted with water and extracted with ethyl acetate. The organic layer was washed with water and brine, dried over anhydrous sodium sulfate, filtered and evaporated under reduced pressure. The residue was purified by silica gel column chromatography using cyclohexane/ethyl acetate 100/0 to 60/40 as eluent to afford 3-(1-{2-[4-(benzyloxy)butoxy]ethyl}-1H-pyrazol-4-yl)-5-[(tert-butyldimethylsilyl) oxy]-1-(oxan-2-yl)-1H-indazole as a pink oil.

LCMS method M: [M+H]⁺=605.5, t_{R}=5.09 min

### Preparation of Intermediate 614: 4-[2-(4-{5-[(tert-butyldimethylsilyl)oxy]-1-(oxan-2-yl)-1H-indazol-3-yl}-1H-pyrazol-1-yl)ethoxy]butan-1-ol

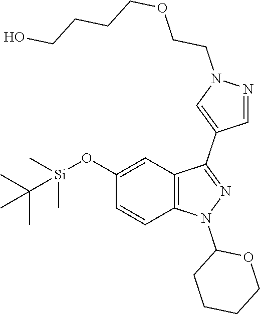 [SMILES: CC(C)(C)[Si](C)(C)Oc1ccc2c(c1)c(-c1cnn(CCOCCCCO)c1)nn2C1CCCCO1]

To a solution of 3-(1-{2-[4-(benzyloxy)butoxy]ethyl}-1H-pyrazol-4-yl)-5-[(tert-butyldimethylsilyl)oxy]-1-(oxan-2-yl)-1H-indazole (1.217 g, 2.012 mmol) in ethanol (20 mL) under argon was added palladium on charcoal 10% (122 mg). The reaction mixture was stirred under hydrogen atmosphere overnight. The reaction mixture was filtered and washed with ethanol. The filtrate was evaporated under reduced pressure to afford 4-[2-(4-{5-[(tert-butyldimethylsilyl)oxy]-1-(oxan-2-yl)-1H-indazol-3-yl}-1H-pyrazol-1-yl)ethoxy]butan-1-ol as a pale brown oil which was used in the next step without further purification.

LCMS method M: [M+H]⁺=515.5, t_{R}=3.23 min

### Preparation of Intermediate 615: 4-[2-(4-{5-[(tert-butyldimethylsilyl)oxy]-1-(oxan-2-yl)-1H-indazol-3-yl}-1H-pyrazol-1-yl)ethoxy]butyl methanesulfonate

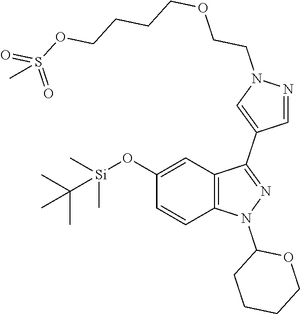 [SMILES: CC(C)(C)[Si](C)(C)Oc1ccc2c(c1)c(-c1cnn(CCOCCCCOS(C)(=O)=O)c1)nn2C1CCCCO1]

To a solution of 4-[2-(4-{5-[(tert-butyldimethylsilyl)oxy]-1-(oxan-2-yl)-1H-indazol-3-yl}-1H-pyrazol-1-yl)ethoxy]butan-1-ol (882 mg, 1.71 mmol) in dichloromethane (15 mL) at 0° C. was added triethylamine (477 μL, 3.42 mmol) then methanesulfonyl chloride (172 μL, 2.22 mmol). The reaction mixture was stirred at RT for 1 h. The reaction mixture was washed with a saturated aqueous NaHCO₃ solution and brine, dried over anhydrous sodium sulfate, filtered and evaporated under reduced pressure to give 4-[2-(4-{5-[(tert-butyldimethylsilyl)oxy]-1-(oxan-2-yl)-1H-indazol-3-yl}-1H-pyrazol-1-yl)ethoxy]butylmethanesulfonate as a green oil.

LCMS method M: [M+H]⁺=593.5, t_{R}=3.81 min

### Preparation of Intermediate 616: 18-(oxan-2-yl)-8,13-dioxa-4,5,18,19-tetraazatetra cyclo[12.5.2.1^{2,5}.0^{17,20}]docosa-1(19),2(22),3,14(21),15,17(20)-hexaene

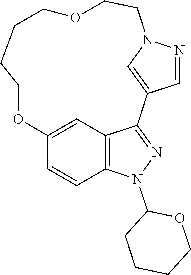 [SMILES: c1cc2c3cc1OCCCCOCCn1cc(cn1)-c3nn2C1CCCCO1]

To a suspension of cesium carbonate (1.32 g, 4.05 mmol) in anhydrous DMF (250 mL) at 80° C. was added dropwise 4-[2-(4-{5-[(tert-butyldimethylsilyl)oxy]-1-(oxan-2-yl)-1H-indazol-3-yl}-1H-pyrazol-1-yl)ethoxy]butylmethanesulfonate (800 mg, 1.32 mmol) in DMF (250 mL). The reaction mixture was stirred at 80° C. for 1 h. The residue was partitioned between ethyl acetate and water. The organic layer was dried over anhydrous sodium sulfate, filtered and evaporated under reduced pressure. The residue was purified by silica gel column chromatography using cyclohexane/(ethyl acetate/ethanol (3/1)) 100/0 to 50/50 as eluent to afford 18-(oxan-2-yl)-8,13-dioxa-4,5,18,19-tetraazatetracyclo[12.5.2.1^{2,5}.0^{17,20}]docosa-1(19), 2(22),3,14(21),15,17(20)-hexaene as a white solid.

LCMS method F: [M+H]⁺=383.4, t_{R}=2.57 min

### Preparation of Example 102: 8,13-dioxa-4,5,18,19-tetraazatetracyclo[12.5.2.1^{2,5}.0^{17,20}]docosa-1(19),2(22),3,14(21),15,17(20)-hexaene

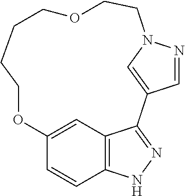 [SMILES: c1cc2[nH]nc3c2cc1OCCCCOCCn1cc-3cn1]

To a solution of 18-(oxan-2-yl)-8,13-dioxa-4,5,18,19-tetraazatetracyclo [12.5.2.1^{2,5}.0^{17,20}] docosa-1(19),2(22),3,14(21),15,17(20)-hexaene (193 mg, 0.505 mmol) in methanol (7.7 mL) and water (1.3 mL) was added p-toluenesulfonic acid monohydrate (479 mg, 2.52 mmol). The reaction mixture was stirred at 65° C. overnight. The reaction mixture was diluted with ethyl acetate and a saturated aqueous NaHCO₃ solution. The phases were separated and the aqueous layer was extracted by ethyl acetate. The combined organic layers were washed brine, dried over anhydrous sodium sulfate, filtered and evaporated under reduced pressure. The residue was purified by silica gel column chromatography using dichloromethane/methanol 100/0 to 95/5 as eluent. the resulting product was triturated with water, filtered and dried to afford 8,13-dioxa-4,5,18,19-tetraazatetracyclo[12.5.2.1^{2,5}.0^{17,20}]docosa-1(19),2(22),3,14(21),15,17(20)-hexaene as a solid.

LCMS method F: [M+H]⁺=299.3, t_{R}=2.01 min

LCMS method G: [M+H]⁺=299.3, t_{R}=1.94 min

¹H NMR (400 MHz, DMSO) 12.74 (1H, s), 8.33 (1H, s), 7.67 (1H, s), 7.41-7.37 (1H, m), 7.19 (1H, d, J=2.1 Hz), 6.94 (1H, dd, J=2.5, 8.9 Hz), 4.42 (2H, dd, J=3.5, 5.2 Hz), 4.22-4.15 (2H, m), 3.60 (2H, t, J=5.2 Hz), 3.71 (2H, t, J=4.4 Hz), 2.10-2.01 (2H, m), 1.76-1.68 (2H, m) ppm.

### Example 103: (6S,12R)-6,12-dimethyl-9,13-dioxa-4,5,18,19,22-pentaazatetracyclo[12.5.2.1^{2,5}.0^{17,20}]docosa-1(19),2(22),3,14(21),15,17(20)-hexaene

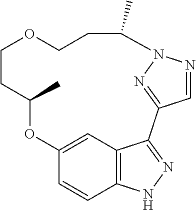 [SMILES: C[C@@H]1CCOCC[C@H](C)n2ncc(n2)-c2n[nH]c3ccc(cc23)O1]

Example 103 is prepared according to the synthesis route described in general scheme D.

### Preparation of Intermediate 617: tert-butyl-[(3S)-3-(4,5-dibromotriazol-2-yl)butoxy]-diphenyl-silane

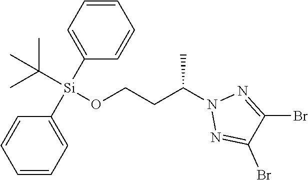 [SMILES: C[C@@H](CCO[Si](c1ccccc1)(c1ccccc1)C(C)(C)C)n1nc(Br)c(Br)n1]

To a solution of 4,5-dibromo-2H-triazole (4.67 g, 20.58 mmol), (2R)-4-[tert-butyl(diphenyl) silyl]oxybutan-2-ol (6.76 g, 20.58 mmol) and triphenylphosphine (8.1 g, 30.87 mmol) in THF (200 mL) at 0° C. was added dropwise a solution of DIAD (6.06 mL, 30.87 mmol) in THF (100 mL). The reaction mixture was stirred at 0° C. for 5 min then RT for 3 h. Ethyl acetate and water were added, the phases were separated and the aqueous layer was extracted with ethyl acetate. The combined organic layers were washed with saturated aqueous NaHCO₃ solution and brine, dried over anhydrous sodium sulfate, filtered and evaporated under reduced pressure. The residue was purified by silica gel column chromatography using cyclohexane/ethyl acetate 100/0 as eluent to afford tert-butyl-[(3S)-3-(4,5-dibromotriazol-2-yl)butoxy]-diphenyl-silane as a colourless oil.

LCMS method M: [M+H]⁺=457.9-460.0-461.9, t_{R}=5.57 min

### Preparation of Intermediate 618: [(3S)-3-(4-bromotriazol-2-yl)butoxy]-tert-butyl-diphenyl-silane

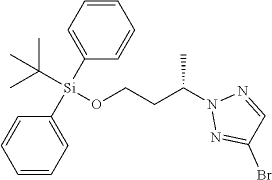 [SMILES: C[C@@H](CCO[Si](c1ccccc1)(c1ccccc1)C(C)(C)C)n1ncc(Br)n1]

A solution of tert-butyl-[(3S)-3-(4,5-dibromotriazol-2-yl)butoxy]-diphenyl-silane (3 g, 5.6 mmol) in diethyl ether (100 mL) at −78° C. was added dropwise n-butyllithium (2.5 M solution in hexane) (2.69 mL, 6.73 mmol). The reaction mixture was warmed to RT and stirred for 2 h. The reaction mixture was cooled to −78° C. and additional n-butyllithium (2.5 M solution in hexane) (1 mL, 2.5 mmol) was added dropwise. The reaction mixture was stirred at RT for 2 h. The reaction mixture was quenched by addition of saturated ammonium chloride solution. The layers were separated and the organic layer was washed with water, dried over anhydrous sodium sulfate, filtered and evaporated under reduced pressure to afford [(3S)-3-(4-bromotriazol-2-yl)butoxy]-tert-butyl-diphenyl-silane as a yellow oil.

LCMS method M: [M−Ph+H]⁺=380.1-382.1, t_{R}=5.12 min

### Preparation of Intermediate 619: (3S)-3-(4-bromotriazol-2-yl)butan-1-ol

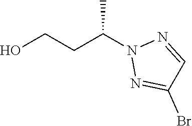

To a solution of [(3S)-3-(4-bromotriazol-2-yl)butoxy]-tert-butyl-diphenyl-silane (2.7 g, 5.89 mmol) in THF (70 mL) at 0° C. was added dropwise tetrabutylammonium fluoride (1.0 M solution in THF) (7.07 mL, 7.07 mmol). The reaction mixture was stirred at RT overnight. Water and ethyl acetate were added, the phases were separated and the aqueous layer was extracted with ethyl acetate. The combined organic phases were washed with brine, dried over anhydrous sodium sulfate, filtered and evaporated under reduced pressure. The residue was purified by silica gel column chromatography using cyclohexane/ethyl acetate 100/0 to 70/30 as eluent to afford (3S)-3-(4-bromotriazol-2-yl)butan-1-ol as a colorless oil.

LCMS method F: [M+H]⁺=220.0-222.0, t_{R}=1.75 min

### Preparation of Intermediate 620: 2-[(1S)-3-[(3S)-3-benzyloxybutoxy]-1-methyl-propyl]-4-bromo-triazole

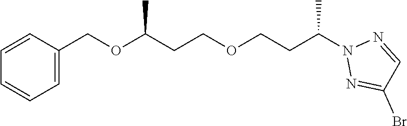 [SMILES: C[C@@H](CCOCC[C@H](C)n1ncc(Br)n1)OCc1ccccc1]

To a suspension of sodium hydride (60% dispersion in mineral oil) (146 mg, 3.64 mmol) in DMF (5 mL) at 0° C. was added dropwise (3S)-3-(4-bromotriazol-2-yl)butan-1-ol (400 mg, 1.82 mmol) in DMF (5 mL). The reaction mixture was stirred at 0° C. for 5 min then at RT for 15 min. The reaction mixture was cooled to 0° C. and a solution of [(3S)-3-benzyloxybutyl] 4-methylbenzenesulfonate (911 mg, 2.74 mmol) in DMF (5 mL) was added. The reaction mixture was stirred at 0° C. for 5 min then at 65° C. overnight. The solvent was evaporated under reduced pressure and the residue was partitioned between ethyl acetate and water. The phases were separated and the organic layer was washed with saturated aqueous NaHCO₃ solution, 1N aqueous hydrochloric acid solution, water and brine. The organic layer was dried over anhydrous sodium sulfate, filtered and evaporated under reduced pressure. The residue was purified by silica gel column chromatography using cyclohexane/ethyl acetate 100/0 to 50/50 as eluent to afford 2-[(1S)-3-[(3S)-3-benzyloxybutoxy]-1-methyl-propyl]-4-bromo-triazole as a colorless oil

LCMS method F: [M+H]⁺=382.1-384.1, t_{R}=3.19 min

### Preparation of Intermediate 621: [3-[2-[(1S)-3-[(3S)-3-benzyloxybutoxy]-1-methyl-propyl]triazol-4-yl]-1-tetrahydropyran-2-yl-indazol-5-yl]oxy-tert-butyl-dimethyl-silane

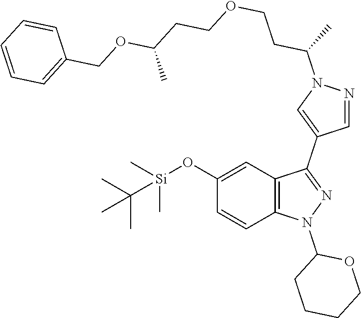 [SMILES: C[C@@H](CCOCC[C@H](C)n1cc(-c2nn(C3CCCCO3)c3ccc(O[Si](C)(C)C(C)(C)C)cc23)cn1)OCc1ccccc1]

To a suspension of tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (777 mg, 1.69 mmol), 2-[(1S)-3-[(3S)-3-benzyloxybutoxy]-1-methyl-propyl]-4-bromo-triazole (590 mg, 1.54 mmol), XPhos (73 mg, 0.154 mmol) and potassium phosphate tribasic (981 mg, 4.62 mmol) in dioxane (19.25 mL) and water (0.75 mL) was added tetrakis(triphenylphosphine)palladium(0) (89 mg, 0.077 mmol). The reaction mixture was stirred at 90° C. for 2 h. The reaction mixture was cooled to RT and partitioned between ethyl acetate and water. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 70/30 as eluent to afford [3-[2-[(1S)-3-[(3S)-3-benzyloxybutoxy]-1-methyl-propyl]triazol-4-yl]-1-tetrahydropyran-2-yl-indazol-5-yl]oxy-tert-butyl-dimethyl-silane as an orange oil.

LCMS method M: [M+H]⁺=634.4, t_{R}=5.86 min

### Preparation of Intermediate 622: (2S)-4-[(3S)-3-[4-[5-[tert-butyl(dimethyl)silyl]oxy-1-tetrahydropyran-2-yl-indazol-3-yl]triazol-2-yl]butoxy]butan-2-ol

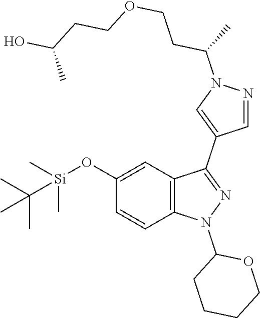 [SMILES: C[C@H](O)CCOCC[C@H](C)n1cc(-c2nn(C3CCCCO3)c3ccc(O[Si](C)(C)C(C)(C)C)cc23)cn1]

To a solution of [3-[2-[(1S)-3-[(3S)-3-benzyloxybutoxy]-1-methyl-propyl]triazol-4-yl]-1-tetra hydropyran-2-yl-indazol-5-yl]oxy-tert-butyl-dimethyl-silane (650 mg, 1.026 mmol) in ethanol (20 mL) at RT was added palladium on carbon 10% (65 mg). The reaction mixture was stirred under hydrogen atmosphere at 50° C. for 2 h. The reaction mixture filtered and the filtrate was concentrated under reduced pressure. The residue was purified by silica gel column chromatography using cyclohexane/ethyl acetate 100/0 to 70/30 as eluent to afford (2S)-4-[(3S)-3-[4-[5-[tert-butyl(dimethyl)silyl]oxy-1-tetrahydropyran-2-yl-indazol-3-yl]triazol-2-yl] butoxy]butan-2-ol as a colorless oil.

LCMS method M: [M+H]⁺=544.5, t_{R}=4.62 min

### Preparation of Intermediate 623: [(1S)-3-[(3S)-3-[4-[5-[tert-butyl(dimethyl)silyl]oxy-1-tetrahydropyran-2-yl-indazol-3-yl]triazol-2-yl]butoxy]-1-methyl-propyl]methane sulfonate

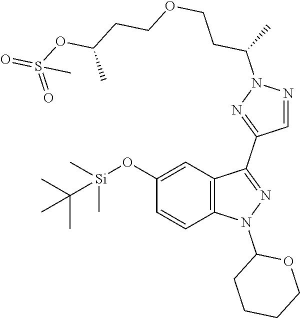 [SMILES: C[C@@H](CCOCC[C@H](C)n1ncc(-c2nn(C3CCCCO3)c3ccc(O[Si](C)(C)C(C)(C)C)cc23)n1)OS(C)(=O)=O]

To a solution of (2S)-4-[(3S)-3-[4-[5-[tert-butyl(dimethyl)silyl]oxy-1-tetrahydropyran-2-yl-indazol-3-yl]triazol-2-yl]butoxy]butan-2-ol (450 mg, 0.829 mmol) in dichloromethane (50 mL) at 0° C. was added triethylamine (231 μL, 1.66 mmol) and methanesulfonyl chloride (76 μL, 0.994 mmol). The reaction mixture was stirred at RT overnight. The reaction mixture was diluted with water and extracted with dichloromethane. The combined organic layers were washed with a saturated aqueous ammonium chloride solution then brine, dried over anhydrous sodium sulfate, filtered and concentrated under reduced pressure to afford [(1S)-3-[(3S)-3-[4-[5-[tert-butyl(dimethyl)silyl]oxy-1-tetrahydropyran-2-yl-indazol-3-yl]triazol-2-yl]butoxy]-1-methyl-propyl]methanesulfonate as an colorless oil which was used in the next step without further purification.

LCMS method F: [M+H]⁺=622.3, t_{R}=3.68 min

### Preparation of Intermediate 624: (6S,12R)-6,12-dimethyl-18-(oxan-2-yl)-9,13-dioxa-4,5,18,19,22-pentaazatetracyclo[12.5.2.1^{2,5}.0^{17,20}]docosa-1(19),2(22),3,14(21),15, 17(20)-hexaene

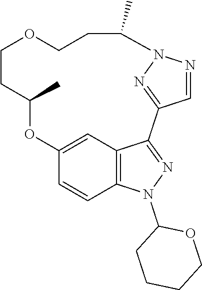 [SMILES: C[C@@H]1CCOCC[C@H](C)n2ncc(n2)-c2nn(C3CCCCO3)c3ccc(cc23)O1]

To a suspension of cesium carbonate (771 mg, 2.37 mmol) in DMF (250 mL) at 85° C. was added dropwise a solution of [(1S)-3-[(3S)-3-[4-[5-[tert-butyl(dimethyl)silyl]oxy-1-tetrahydro pyran-2-yl-indazol-3-yl]triazol-2-yl]butoxy]-1-methyl-propyl]methanesulfonate (368 mg, 0.592 mmol) in DMF (350 mL). The reaction mixture was stirred at 85° C. for 16 h. The reaction mixture was cooled to RT, filtered and concentrated under reduced pressure. The residue was purified by silica gel column chromatography using cyclohexane/ethyl acetate 100/0 to 70/30 as eluent to afford (6S,12R)-6,12-dimethyl-18-(oxan-2-yl)-9,13-dioxa-4,5,18,19,22-pentaazatetracyclo[12.5.2.1^{2,5}.0^{17,20}]docosa-1(19),2(22),3,14(21),15,17(20)-hexaene as a white gummy solid.

LCMS method F: [M+H]⁺=412.4, t_{R}=3.16 min

### Preparation of Example 103: (6S,12R)-6,12-dimethyl-9,13-dioxa-4,5,18,19,22-pentaazatetracyclo[12.5.2.1^{2,5}.0^{17,20}] docosa-1(19),2(22),3,14(21),15,17(20)-hexaene

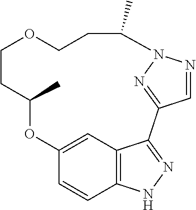 [SMILES: C[C@@H]1CCOCC[C@H](C)n2ncc(n2)-c2n[nH]c3ccc(cc23)O1]

To a solution of (6S,12R)-6,12-dimethyl-18-(oxan-2-yl)-9,13-dioxa-4,5,18,19,22-pentaazatetra cyclo[12.5.2.1^{2,5}.0^{17,20}]docosa-1(19),2(22),3,14(21),15,17(20)-hexaene (85 mg, 0.206 mmol) in methanol (6.66 mL) and water (1.33 mL) was added p-toluenesulfonic acid monohydrate (196 mg, 1.03 mmol). The reaction mixture was stirred at 50° C. for 16 h. Solvents were concentrated under reduced pressure and the residue was partitioned between ethyl acetate and saturated aqueous NaHCO₃ solution. The aqueous layer was extracted with ethyl acetate and the combined organic layers were washed with brine, dried over anhydrous sodium sulfate, filtered and evaporated under reduced pressure. The residue was purified by silica gel column chromatography using cyclohexane/ethyl acetate 100/0 to 70/30 as eluent. The resulting product was triturated with diisopropylether, filtered and dried to afford (6S,12R)-6,12-dimethyl-9,13-dioxa-4,5,18,19,22-pentaazatetracyclo[12.5.2.1^{2,5}.0^{17,20}]docosa-1(19),2(22),3, 14(21),15,17(20)-hexaene as a solid.

LCMS method F: [M+H]⁺=328.1, t_{R}=2.47 min

LCMS method G: [M+H]⁺=328.3, t_{R}=2.36 min

¹H NMR (400 MHz, DMSO) 13.04 (1H, s), 8.12-8.07 (2H, m), 7.45-7.42 (1H, m), 6.97 (1H, dd, J=2.5, 8.9 Hz), 5.02-4.96 (1H, m), 4.67-4.61 (1H, m), 4.02-3.96 (1H, m), 3.78-3.73 (1H, m), 3.62-3.52 (1H, m), 3.44 (1H, q, J=7.3 Hz), 2.43-2.36 (2H, m), 2.20-2.11 (1H, m), 1.56 (3H, d, J=7.0 Hz), 1.40-1.37 (4H, m) ppm.

### Example 104: 10,13-dioxa-4,5,18,19-tetraazatetracyclo[12.5.2.1^{2,5}.0^{17,20}]docosa-1(19), 2(22),3,14(21),15,17(20)-hexaene

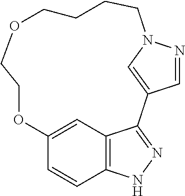 [SMILES: c1cc2[nH]nc3c2cc1OCCOCCCCn1cc-3cn1]

Example 104 is prepared according to the synthesis route described in general scheme D.

### Preparation of Intermediate 625: 2-(4-bromobutoxy)ethoxymethylbenzene

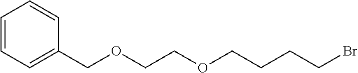 [SMILES: BrCCCCOCCOCc1ccccc1]

To a solution of 2-benzyloxyethanol (4.46 g, 29.3 mmol) in toluene (35 mL) and water (35 mL) was added sodium hydroxide (9.37 g, 234.4 mmol), tetrabutyl ammonium hydrogen sulfate (9.94 g, 29.3 mmol) and 1,4-dibromobutane (9.48 g, 43.95 mmol). The reaction mixture was stirred RT overnight. 1N aqueous hydrochloric acid solution was added and the solution was extracted with ethyl acetate. The combined organic layers were dried over anhydrous sodium sulfate, filtered and evaporated under reduced pressure. The residue was purified by silica gel column chromatography using cyclohexane/ethyl acetate 100/0 60/40 as eluent to afford 2-(4-bromobutoxy)ethoxymethylbenzene as a light yellow oil.

LCMS method F: [M+H]⁺=287.1-289.1, t_{R}=2.88 min

### Preparation of Intermediate 626: 1-[4-(2-benzyloxyethoxy)butyl]-4-bromo-pyrazole

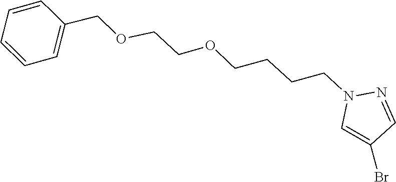 [SMILES: Brc1cnn(CCCCOCCOCc2ccccc2)c1]

To a solution of 4-bromo-1H-pyrazole (2.21 g, 15.07 mmol) in acetonitrile (75 mL) was added cesium carbonate (6.38 mg, 19.6 mmol) and 2-(4-bromobutoxy)ethoxymethylbenzene (4.33 g, 15.07 mmol). The reaction mixture was stirred at 85° C. for 4 h. The reaction mixture was cooled to RT, water and ethyl acetate were added and the layers were separated. The aqueous layer was extracted with ethyl acetate and the combined organic layers were dried over anhydrous sodium sulfate, filtered and evaporated under reduced pressure. The residue was purified by silica gel column chromatography using cyclohexane/ethyl acetate 100/0 to 50/50 as eluent to afford 1-[4-(2-benzyloxyethoxy)butyl]-4-bromo-pyrazole as a colorless liquid.

LCMS method F: [M+H]⁺=353.1-355.1, t_{R}=2.78 min

### Preparation of Intermediate 627: 3-[1-[4-(2-benzyloxyethoxy)butyl]pyrazol-4-yl]-1-tetrahydropyran-2-yl-indazol-5-yl]oxy-tert-butyl-dimethyl-silane

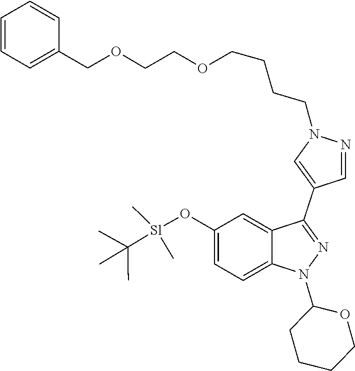

To a suspension of 1-[4-(2-benzyloxyethoxy)butyl]-4-bromo-pyrazole (2.97 g, 8.4 mmol) in dioxane (62 mL) and water (3 mL) was added tert-butyl-dimethyl-[1-tetrahydropyran-2-yl-3-(4,4,5,5-tetramethyl-1,3,2-dioxaborolan-2-yl)indazol-5-yl]oxy-silane (intermediate 61) (5.01 g, 10.92 mmol) and potassium phosphate tribasic (5.35 g, 25.22 mmol). The reaction mixture was purged with argon for 10 min then tetrakis(triphenylphosphine)palladium(0) (485 mg, 0.42 mmol) and Xphos (400 mg, 0.84 mmol) were added. The resulting mixture was stirred at 90° C. overnight. The mixture was cooled to RT and the reaction was diluted with water and ethyl acetate. The phases were separated and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 60/40 as eluent to afford 3-[1-[4-(2-benzyloxyethoxy)butyl]pyrazol-4-yl]-1-tetrahydropyran-2-yl-indazol-5-yl]oxy-tert-butyl-dimethyl-silane (1.92 mg, 3.17 mmol) as a dark orange oil.

LCMS method F: [M+H]⁺=605.3, t_{R}=3.73 min

### Preparation of Intermediate 628: 2-[4-[4-[5-[tert-butyl(dimethyl)silyl]oxy-1-tetra hydropyran-2-yl-indazol-3-yl]pyrazol-1-yl]butoxy]ethanol

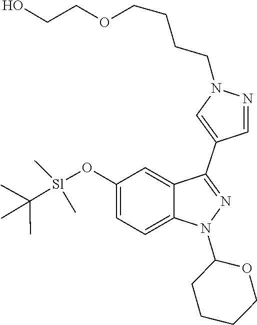

To a suspension of [3-[1-[4-(2-benzyloxyethoxy)butyl]pyrazol-4-yl]-1-tetrahydropyran-2-yl-indazol-5-yl]oxy-tert-butyl-dimethyl-silane (1.92 g, 3.17 mmol) in ethanol (40 mL) was added palladium on charcoal 10% (192 mg). The reaction mixture was stirred under hydrogen atmosphere overnight. The reaction mixture was filtered and washed with ethanol. The filtrate was evaporated under reduced pressure. The residue was purified by silica gel column chromatography using dichloromethane/methanol 100/0 to 90/10 as eluent to afford 2-[4-[4-[5-[tert-butyl(dimethyl)silyl]oxy-1-tetrahydropyran-2-yl-indazol-3-yl]pyrazol-1-yl]butoxy] ethanol (1.32 g, 2.56 mmol) as a brown oil.

LCMS method F: [M+H]⁺=515.3, t_{R}=3.23 min

### Preparation of Intermediate 629: 2-[4-[4-[5-[tert-butyl(dimethyl)silyl]oxy-1-tetra hydropyran-2-yl-indazol-3-yl]pyrazol-1-yl]butoxy]ethyl methanesulfonate

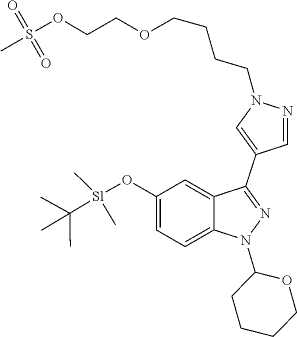

To a solution of 2-[4-[4-[5-[tert-butyl(dimethyl)silyl]oxy-1-tetrahydropyran-2-yl-indazol-3-yl]pyrazol-1-yl]butoxy]ethanol (1.32 g, 2.56 mmol) in dichloromethane (25 mL) at 0° C. was added triethylamine (713 μL, 5.12 mmol) and methanesulfonyl chloride (257 μL, 3.33 mmol). The reaction mixture was stirred at 0° C. for 10 min then RT for 1 h. Water and dichloromethane were added and the phases were separated.

The organic layer was dried over anhydrous sodium sulfate, filtered and evaporated under reduced pressure to afford 2-[4-[4-[5-[tert-butyl(dimethyl)silyl]oxy-1-tetrahydropyran-2-yl-indazol-3-yl]pyrazol-1-yl]butoxy]ethylmethanesulfonate as a colorless oil which was used without further purification.

LCMS method F: [M+H]⁺=593.3, t_{R}=3.38 min

### Preparation of Intermediate 630: 18-(oxan-2-yl)-10,13-dioxa-4,5,18,19-tetraazatetra cyclo[12.5.2.1^{2,5}.0^{17,20}]docosa-1(19),2(22),3,14(21),15,17(20)-hexaene

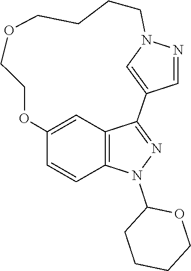 [SMILES: c1cc2c3cc1OCCOCCCCn1cc(cn1)-c3nn2C1CCCCO1]

To a suspension of cesium carbonate (2.50 g, 7.68 mmol) in anhydrous DMF (500 mL) at 80° C. was added dropwise 2-[4-[4-[5-[tert-butyl(dimethyl)silyl]oxy-1-tetrahydropyran-2-yl-indazol-3-yl]pyrazol-1-yl]butoxy]ethyl methanesulfonate (1.67 g, 2.56 mmol) in DMF (500 mL). The reaction mixture was stirred at 80° C. for 1 h. The residue was partitioned between ethyl acetate and water. The phases were separated. The organic layer dried over anhydrous sodium sulfate, filtered and evaporated under reduced pressure. The residue was purified by silica gel column chromatography using cyclohexane/(ethyl acetate/ethanol (3/1)) as eluent to afford 18-(oxan-2-yl)-10,13-dioxa-4,5,18,19-tetraazatetracyclo[12.5.2.1^{2,5}.0^{17,20}]docosa-1(19),2(22),3,14(21), 15,17(20)-hexaene as a white solid.

LCMS method F: [M+H]⁺=383.2, t_{R}=2.60 min

### Preparation of Example 104: 10,13-dioxa-4,5,18,19-tetraazatetracyclo[12.5.2.1^{2,5}.0^{17,20}]docosa-1(19),2(22),3,14(21),15,17(20)-hexaene

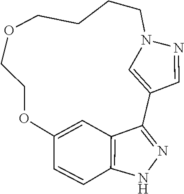 [SMILES: c1cc2[nH]nc3c2cc1OCCOCCCCn1cc-3cn1]

To a solution of 18-(oxan-2-yl)-10,13-dioxa-4,5,18,19-tetraazatetracyclo[12.5.2.1^{2,5}.0^{17,20}] docosa-1(19),2(22),3,14(21),15,17(20)-hexaene (140 mg, 0.366 mmol) in methanol (5.5 mL) and water (1 mL) was added p-toluenesulfonic acid monohydrate (347 mg, 1.83 mmol). The reaction mixture was stirred at 65° C. overnight. The reaction mixture was diluted with ethyl acetate and a saturated aqueous NaHCO₃ solution. The phases were separated and the aqueous layer was extracted with ethyl acetate. The combined organic layer was washed with brine, dried over anhydrous sodium sulfate, filtered and evaporated under reduced pressure. The residue was purified by silica gel column chromatography using dichloromethane/methanol 100/0 to 90/10 as eluent. The resulting solid was triturated with diethyl ether, filtered and dried to afford 10,13-dioxa-4,5,18,19-tetraazatetracyclo[12.5.2.1^{2,5}.0^{17,20}]docosa-1(19),2(22),3, 14(21),15,17(20)-hexaene as a solid.

LCMS method F: [M+H]⁺=299.3, t_{R}=1.98 min

LCMS method G: [M+H]⁺=299.3, t_{R}=2.02 min

¹H NMR (400 MHz, DMSO) 12.70 (1H, s), 8.47 (1H, s), 8.13 (1H, m), 7.66 (1H, s), 7.37-7.33 (1H, m), 6.97 (1H, dd, J=2.4, 8.8 Hz), 4.40-4.36 (2H, m), 4.27-4.22 (2H, m), 3.77-3.74 (2H, m), 3.50 (2H, m), 2.21-2.13 (2H, m), 1.39-1.30 (2H, m) ppm.

### Examples 105 and 106: (6R*,13R)-6-fluoro-13-methyl-8,11,14-trioxa-4,5,19,20-tetraaza tetracyclo[13.5.2.1^{2,5}.0^{18,21}]tricosa-1(20),2(23),3,15(22),16,18(21)-hexaene and (6S*,13R)-6-fluoro-13-methyl-8,11,14-trioxa-4,5,19,20-tetraazatetra cyclo[13.5.2.1^{2,5}.0^{18,21}]tricosa-1(20),2(23),3,15(22),16,18(21)-hexaene

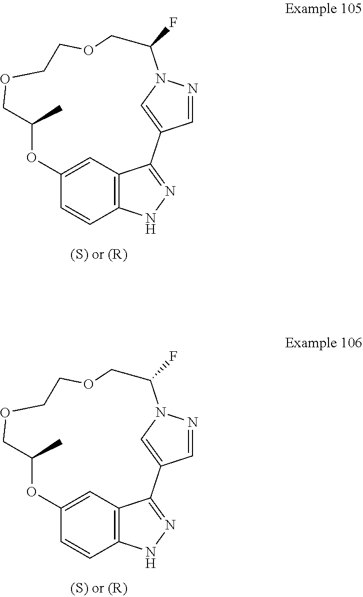 [SMILES: C[C@@H]1COCCOC[C@@H](F)n2cc(cn2)-c2n[nH]c3ccc(cc23)O1 ; C[C@@H]1COCCOC[C@H](F)n2cc(cn2)-c2n[nH]c3ccc(cc23)O1]

Example 105 and example 106 are prepared according to the synthesis route described in general scheme D.

### Preparation of Intermediate 631: 2-(4-bromopyrazol-1-yl)-2-fluoro-acetic acid

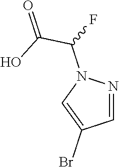 [SMILES: O=C(O)C(F)n1cc(Br)cn1]

To a solution of 4-bromo-1H-pyrazole (1.02 g, 7 mmol) in DMF (50 mL) at 0° C. was added sodium hydride (60% dispersion in mineral oil) (616 mg, 15.4 mmol). The reaction mixture was stirred at 0° C. for 10 min then ethyl 2-bromo-2-fluoro-acetate (0.91 mL, 7.7 mmol) was added. The reaction mixture was stirred at RT for 1 h. The reaction mixture was quenched with water then extracted with ethyl acetate. The combined organic layers were washed with a 1N aqueous NaOH solution. The pH of the combined aqueous layers was adjusted at pH 1 with a 1N aqueous HCl solution. The aqueous layer was extracted with ethyl acetate. The organic layer was washed with water, brine, dried over anhydrous sodium sulfate, filtered and evaporated to afford 2-(4-bromopyrazol-1-yl)-2-fluoro-acetic acid as a pale yellow oil which crystallized. The crude product was used in the next step without any further purification.

LCMS method F: [M+H]⁺=223.0-225.0, t_{R}=1.62 min

### Preparation of Intermediate 632: 2-(4-bromopyrazol-1-yl)-2-fluoro-ethanol

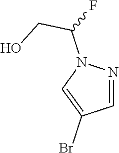

To a degassed solution of 2-(4-bromopyrazol-1-yl)-2-fluoro-acetic acid (1.53 g, 6.86 mmol) in THF (46 mL) was added 1 M borane dimethyl sulfide solution in Me-THF (8.1 mL, 8.1 mmol) at 0° C. The reaction mixture was stirred at room temperature overnight. The reaction mixture was quenched with methanol at 0° C., then water and ethyl acetate were added. The aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford 2-(4-bromopyrazol-1-yl)-2-fluoro-ethanol as a colorless oil.

LCMS method F: [M+H]⁺=209.1-211.1, t_{R}=1.49 min

### Preparation of Intermediate 633: 1-[2-[2-[(2S)-2-benzyloxypropoxy]ethoxy]-1-fluoro-ethyl]-4-bromo-pyrazole

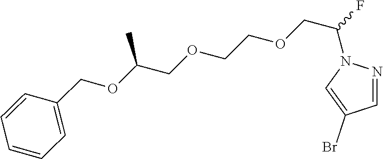 [SMILES: C[C@@H](COCCOCC(F)n1cc(Br)cn1)OCc1ccccc1]

To a degassed solution of 2-(4-bromopyrazol-1-yl)-2-fluoro-ethanol (678 mg, 3.24 mmol) in dry DMF (6 mL) at 0° C. was added sodium hydride (60% dispersion in mineral oil) (151 mg, 3.78 mmol). The reaction mixture was stirred at RT for 10 min. A solution of 2-[(2S)-2-benzyloxypropoxy]ethyl methanesulfonate (the (S)-enantiomer of intermediate 387) (785 mg, 2.52 mmol) in dry DMF (5 mL) was added and the reaction mixture was stirred at RT overnight. The reaction mixture was quenched with water and extracted with ethyl acetate. The combined organic layers were washed with water, brine, dried over anhydrous sodium sulfate, filtered and evaporated under reduced pressure. The residue was purified by silica gel column chromatography using cyclohexane/ethyl acetate 100/0 to 80/20 as eluent to afford 1-[2-[2-[(2S)-2-benzyloxypropoxy]ethoxy]-1-fluoro-ethyl]-4-bromo-pyrazole as a colorless oil.

LCMS method F: [M+H]⁺=401.1-403.1, t_{R}=2.89 min

### Preparation of Intermediate 634: [3-[1-[2-[2-[(2S)-2-benzyloxypropoxy]ethoxy]-1-fluoro-ethyl]pyrazol-4-yl]-1-tetrahydropyran-2-yl-indazol-5-yl]oxy-tert-butyl-dimethyl-silane

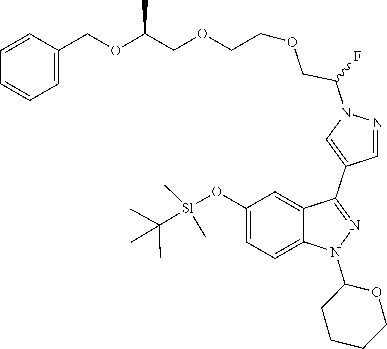

To a suspension of 1-[2-[2-[(2S)-2-benzyloxypropoxy]ethoxy]-1-fluoro-ethyl]-4-bromo-pyrazole (209 mg, 0.52 mmol) in dioxane (3 mL) and water (0.14 mL) was added tert-butyl-dimethyl-[1-tetrahydropyran-2-yl-3-(4,4,5,5-tetramethyl-1,3,2-dioxaborolan-2-yl)indazol-5-yl]oxy-silane intermediate 61 (334 mg, 0.73 mmol) and potassium phosphate tribasic (330 mg, 1.56 mmol). The reaction mixture was degassed by with argon for 10 min then tetrakis(triphenylphosphine)palladium(0) (30 mg, 0.026 mmol) and Xphos (25 mg, 0.052 mmol) were added. The reaction mixture was heated at 90° C. under microwave irradiation for 2 h. The mixture was cooled to RT and filtered through a Celite pad. The filtrate was diluted with water and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 80/20 as eluent to afford [3-[1-[2-[2-[(2S)-2-benzyloxypropoxy]ethoxy]-1-fluoro-ethyl]pyrazol-4-yl]-1-tetrahydropyran-2-yl-indazol-5-yl]oxy-tert-butyl-dimethyl-silane as a pale brown oil.

LCMS method F: [M+H]⁺=654.1, t_{R}=3.94 min

### Preparation of Intermediate 635: (2S)-1-[2-[2-[4-[5-[tert-butyl(dimethyl)silyl]oxy-1-tetrahydropyran-2-yl-indazol-3-yl]pyrazol-1-yl]-2-fluoro-ethoxy]ethoxy]propan-2-ol

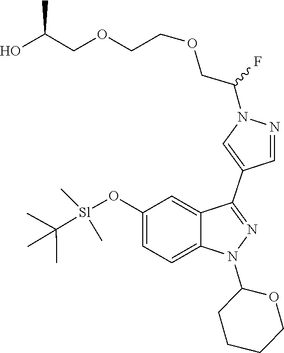

To a solution of [3-[1-[2-[2-[(2S)-2-benzyloxypropoxy]ethoxy]-1-fluoro-ethyl]pyrazol-4-yl]-1-tetrahydropyran-2-yl-indazol-5-yl]oxy-tert-butyl-dimethyl-silane (211 mg, 0.32 mmol) in ethyl acetate (6 mL) at RT was added palladium on carbon 10 wt. % loading (21 mg). The reaction mixture was stirred under hydrogen atmosphere at RT overnight. The reaction mixture was filtered and the filtrate was concentrated under reduced pressure. The residue was purified by silica gel column chromatography using cyclohexane/ethyl acetate 100/0 to 50/50 as eluent to afford (2S)-1-[2-[2-[4-[5-[tert-butyl(dimethyl)silyl]oxy-1-tetrahydropyran-2-yl-indazol-3-yl]pyrazol-1-yl]-2-fluoro-ethoxy]ethoxy]propan-2-ol as a colorless oil.

LCMS method F: [M+H]⁺=563.5, t_{R}=3.43 min

### Preparation of Intermediate 636: [(1S)-2-[2-[2-[4-[5-[tert-butyl(dimethyl)silyl]oxy-1-tetrahydropyran-2-yl-indazol-3-yl]pyrazol-1-yl]-2-fluoro-ethoxy]ethoxy]-1-methyl-ethyl]methanesulfonate

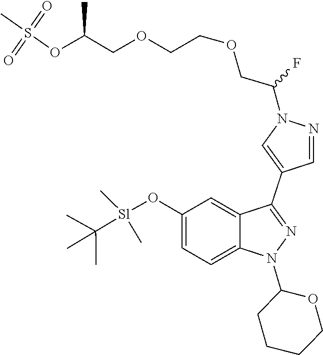

To a solution of (2S)-1-[2-[2-[4-[5-[tert-butyl(dimethyl)silyl]oxy-1-tetrahydropyran-2-yl-indazol-3-yl]pyrazol-1-yl]-2-fluoro-ethoxy]ethoxy]propan-2-ol (139 mg, 0.25 mmol) in dichloromethane (3 mL) was added triethylamine (70 μL, 0.5 mol) and methanesulfonyl chloride (23 μL, 0.3 mmol). The reaction mixture was stirred at RT overnight. Additional triethylamine (31 μL, 0.22 mol) and methanesulfonyl chloride (8 μL, 0.1 mmol) were added and the reaction was stirred at RT for 2 h. The reaction mixture was diluted with water and extracted with dichloromethane. The combined organic layers were washed with a saturated aqueous NH₄Cl solution and brine, dried over anhydrous sodium sulfate, filtered and evaporated under reduced pressure to afford [(1S)-2-[2-[2-[4-[5-[tert-butyl(dimethyl)silyl]oxy-1-tetrahydropyran-2-yl-indazol-3-yl]pyrazol-1-yl]-2-fluoro-ethoxy]ethoxy]-1-methyl-ethyl] methanesulfonate as a colorless oil.

LCMS method F: [M+H]⁺=641.4, t_{R}=3.58 min

### Preparation of Intermediate 637: (13R)-6-fluoro-13-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16, 18(21)-hexaene

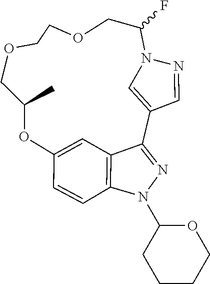 [SMILES: C[C@@H]1COCCOCC(F)n2cc(cn2)-c2nn(C3CCCCO3)c3ccc(cc23)O1]

To a suspension of cesium carbonate (244 mg, 0.75 mmol) in DMF (85 mL) at 85° C. was added dropwise during 2 h, [(1S)-2-[2-[2-[4-[5-[tert-butyl(dimethyl)silyl]oxy-1-tetrahydropyran-2-yl-indazol-3-yl]pyrazol-1-yl]-2-fluoro-ethoxy]ethoxy]-1-methyl-ethyl] methanesulfonate (173 mg, 0.25 mmol) in DMF (85 mL). After addition the reaction mixture was stirred at 85° C. overnight. The reaction mixture was concentrated under reduced pressure and the resulting residue was diluted with water and extracted with ethyl acetate. The combined organic layers were washed with brine, dried over anhydrous sodium sulfate, filtered and evaporated under reduced pressure. The residue was purified by silica gel column chromatography using cyclohexane/(ethyl acetate/ethanol 3/1) 100/0 to 80/20 as eluent to afford (13R)-6-fluoro-13-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene as a red/orange oil.

LCMS method F: [M+H]⁺=431.2, t_{R}=2.78 min

### Preparation of Example 105 and Example 106: (6R*,13R)-6-fluoro-13-methyl-8,11,14-trioxa-4,5,19,20-tetraazatetracyclo[13.5.2.1^{2,5}.0^{18,21}]tricosa-1(20),2(23),3, 15(22),16,18(21)-hexaene and (6S*,13R)-6-fluoro-13-methyl-8,11,14-trioxa-4,5,19, 20-tetraazatetracyclo[13.5.2.1^{2,5}.0^{18,21}]tricosa-1(20),2(23),3,15(22),16,18(21)-hexaene

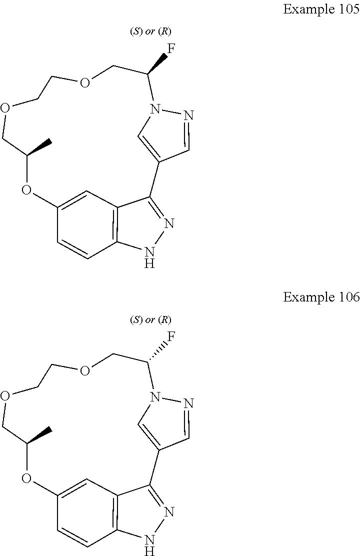 [SMILES: C[C@@H]1COCCOC[C@@H](F)n2cc(cn2)-c2n[nH]c3ccc(cc23)O1 ; C[C@@H]1COCCOC[C@H](F)n2cc(cn2)-c2n[nH]c3ccc(cc23)O1]

To a solution of (13R)-6-fluoro-13-methyl-19-(oxan-2-yl)-8,11,14-trioxa-4,5,19,20-tetraazatetracyclo[13.5.2.1^{2,5}.0^{18,21}]tricosa-1(20),2(23),3,15(22),16,18(21)-hexaene (60 mg, 0.14 mmol) in dioxane (3.5 mL) was added 4M HCl in dioxane (0.35 mL, 1.4 mmol). The reaction mixture was stirred at RT for 48 h. Additional 4M HCl in dioxane (0.18 mL, 0.7 mmol) was added and the reaction mixture was stirred at RT for 24 h. Additional 4M HCl in dioxane (0.18 mL, 0.7 mmol) was added and the reaction mixture was stirred for 4 h. The reaction mixture was poured into a saturated aqueous NaHCO₃ solution and extracted with dichloromethane. The combined organic layers were washed with brine, dried over anhydrous sodium sulfate, filtered and evaporated under reduced pressure. The residue was purified by preparative TLC using ethyl acetate 100% as eluent. The two diastereoisomers were separated to afford:
(6R or S,13R)-6-fluoro-13-methyl-8,11,14-trioxa-4,5,19,20-tetraazatetracyclo [13.5.2.1^{2,5}.0^{18,21}]tricosa-1(20),2(23),3,15(22),16,18(21)-hexaene example 105 as a white solid (Isomer 1)*.

LCMS method F: [M+H]⁺=347.2, t_{R}=2.22 min

LCMS method G: [M+H]⁺=347.3, t_{R}=2.13 min

¹H NMR (400 MHz, CDCl₃): 9.01 (1H, s), 8.18 (1H, s), 7.73 (1H, s), 7.40 (1H, d, J=9 Hz), 7.11 (1H, dd, J=2.2, 9.0 Hz), 6.51-6.39 (1H, m), 4.36 (1H, m), 4.29-4.13 (2H, m), 4.04-3.96 (2H, m), 3.92-3.82 (2H, m), 3.75-3.72 (1H, m), 3.55 (1H, dd, J=4.0, 9.9 Hz), 1.51 (3H, d, J=7 Hz) ppm. One labile proton was not observed.
(6R or S,13R)-6-fluoro-13-methyl-8,11,14-trioxa-4,5,19,20-tetraazatetracyclo [13.5.2.1^{2,5}.0^{18,21}] tricosa-1(20),2(23),3,15(22),16,18(21)-hexaene example 106 as a white solid (Isomer 2)*.

LCMS method F: [M+H]⁺=347.3, t_{R}=2.18 min

LCMS method G: [M+H]⁺=347.3, t_{R}=2.10 min

¹H NMR (400 MHz, CDCl₃): 9.11 (1H, s), 8.22 (1H, s), 8.06 (1H, d, J=2.3 Hz), 7.38 (1H, d, J=9.1 Hz), 7.13 (1H, dd, J=2.3, 8.9 Hz), 6.49-6.37 (1H, m), 4.57 (1H, m), 4.38 (1H, m), 3.98-3.83 (5H, m), 3.67 (1H, m), 3.59 (1H, dd, J=10.2, 2.1 Hz), 1.46 (3H, d, J=7 Hz) ppm. *The absolute configuration of the fluoride substituent in position 6 is unknown.

### Example 107: (12S)-12-methyl(9,9,10,10-²H₄)-8,11,14-trioxa-4,5,19,20-tetraazatetracyclo [13.5.2.1^{2,5}.0^{18,21}]tricosa-1(20),2(23),3,15,17,21-hexaene

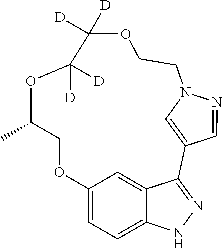 [SMILES: [2H]C1([2H])OCCn2cc(cn2)-c2n[nH]c3ccc(cc23)OC[C@H](C)OC1([2H])[2H]]

Example 107 is prepared according to the synthesis route described in general scheme D, following the reaction procedures described for example 54, using 4-bromo-1H-pyrazole, (2S)-1-benzyloxypropan-2-ol intermediate 300, ethylene-d4 glycol, 2-(2-bromoethoxy)tetrahydro pyran and tert-butyl-dimethyl-[1-tetrahydropyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=333.1, t_{R}=2.03 min

LCMS method G: [M+H]⁺=333.2, t_{R}=2.01 min

¹H NMR (400 MHz, CDCl₃): 8.56 (1H, d, J=0.8 Hz), 8.01 (1H, d, J=0.8 Hz), 7.96 (1H, m), 7.35 (1H, dd, J=0.6, 8.9 Hz), 7.11 (1H, dd, J=2.5, 8.9 Hz), 4.48 (2H, t, J=4.6 Hz), 4.37 (1H, AB syst, J_{AB1}=13.5 Hz, J_{AB2}=3.5 Hz), 4.33 (1H, AB syst, J_{AB1}=13.5 Hz, J_{AB2}=3.5 Hz), 3.86 (3H, m), 1.35 (3H, d, J=6.5 Hz) ppm.

### Example 108: 8,14-dioxa-4,5,19,20-tetraazatetracyclo[13.5.2.1^{2,5}.0^{18,21}]tricosa-1(20),2(23), 3,15(22),16,18(21)-hexaene

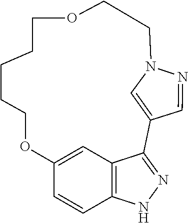 [SMILES: c1cc2[nH]nc3c2cc1OCCCCCOCCn1cc-3cn1]

Example 108 is prepared according to the synthesis route described in general scheme D, following the reaction procedures described for example 102 and using 5-(benzyloxy)pentan-1-ol, 2-(4-bromo-1H-pyrazol-1-yl)ethan-1-ol and tert-butyl-dimethyl-[1-tetrahydropyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=313.1, t_{R}=2.25 min

LCMS method G: [M+H]⁺=313.2, t_{R}=2.17 min

¹H NMR (400 MHz, DMSO) 12.77 (1H, s), 8.36 (1H, s), 7.78 (1H, s), 7.43-7.40 (1H, m), 7.13 (1H, d, J=2.3 Hz), 6.97 (1H, dd, J=2.3, 8.9 Hz), 4.39-4.35 (2H, m), 4.19 (2H, t, J=7.4 Hz), 3.83-3.79 (2H, m), 3.57-3.52 (2H, m), 1.89-1.68 (4H, m), 1.62-1.56 (2H, m) ppm.

### Example 109: (6R)-11,11-difluoro-6-methyl-9,13-dioxa-4,5,18,19-tetraazatetracyclo [12.5.2.1^{2,5}.0^{17,20}]docosa-1(19),2(22),3,14(21),15,17(20)-hexaene

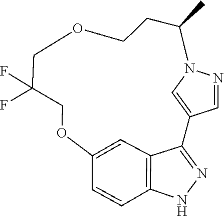 [SMILES: C[C@@H]1CCOCC(F)(F)COc2ccc3[nH]nc(c3c2)-c2cnn1c2]

Example 109 is prepared according to the synthesis route described in general scheme E, following the reaction procedures described for example 42, using (3S)-3-benzyloxybutan-1-ol, 2,2-difluoropropane-1,3-diol and 1-tetrahydropyran-2-yl-3-[1-(2-trimethylsilylethoxy methyl)pyrazol-4-yl]indazol-5-ol intermediate 229 as starting materials.

LCMS method F: [M+H]⁺=349.3, t_{R}=2.14 min

LCMS method G: [M+H]⁺=349.3, t_{R}=2.13 min

¹H NMR (400 MHz, DMSO) 12.79 (1H, s), 8.55 (1H, s), 7.68 (1H, s), 7.43-7.39 (2H, m), 7.05 (1H, dd, J=2.5, 8.9 Hz), 4.70-4.52 (3H, m), 3.83-3.68 (2H, m), 3.43 (1H, m), 3.34-3.33 (1H, m), 2.35-2.30 (1H, m), 2.21-2.14 (1H, m), 1.55 (3H, d, J=6.6 Hz) ppm.

### Example 110: (12S)-12-methyl(6,6,7,7-2H4)-8,11,14-trioxa-4,5,19,20-tetraazatetracyclo [13.5.2.1^{2,5}.0^{18,21}]tricosa-1(20),2(23),3,15(22),16,18(21)-hexaene

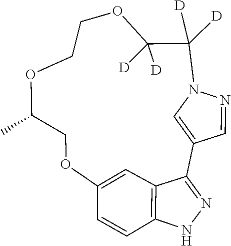 [SMILES: [2H]C1([2H])OCCO[C@@H](C)COc2ccc3[nH]nc(c3c2)-c2cnn(c2)C1([2H])[2H]]

Example 110 is prepared according to the synthesis route described in general scheme D, following the reaction procedures described for example 54, using (2S)-1-benzyloxypropan-2-ol intermediate 300, ethylene-d4 glycol, 2-(2-bromoethoxy)tetrahydropyran, 4-bromo-1H-pyrazole and tert-butyl-dimethyl-[1-tetrahydropyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=333.3, t_{R}=1.98 min

LCMS method G: [M+H]⁺=333.2, t_{R}=2.00 min

¹H NMR (400 MHz, CDCl₃): 8.56 (1H, m), 8.01 (1H, d, J=0.8 Hz), 7.96 (1H, d, J=2.4 Hz), 7.35 (1H, dd, J=0.4, 9.2 Hz), 7.12 (1H, dd, J=2.3, 8.9 Hz), 4.49 (1H, t, J=4.5 Hz), 4.37 (1H, AB syst, J_{AB}=3.6, 13.2 Hz), 4.32 (1H, AB syst, J_{AB}=3.6, 13.2 Hz), 3.92-3.84 (2H, m), 3.76-3.59 (2H, m), 1.36 (3H, d, J=6.3 Hz) ppm.

### Example 111: 11,11-difluoro-9,13-dioxa-4,5,18,19-tetraazatetracyclo[12.5.2.1^{2,5}.0^{17,20}] docosa-1(19),2(22),3,14(21),15,17(20)-hexaene

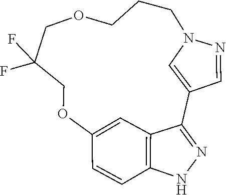 [SMILES: FC1(F)COCCCn2cc(cn2)-c2n[nH]c3ccc(cc23)OC1]

Example 111 is prepared according to the synthesis route described in general scheme E, following the reaction procedures described for example 42, using 2,2-difluoropropane-1,3-diol, 3-benzyloxypropyl 4-methylbenzenesulfonate intermediate 431 and 1-tetrahydropyran-2-yl-3-[1-(2-trimethylsilylethoxymethyl)pyrazol-4-yl]indazol-5-ol intermediate 229 as starting materials.

LCMS method F: [M+H]⁺=335.3, t_{R}=2.00 min

LCMS method G: [M+H]⁺=335.2, t_{R}=2.00 min

¹H NMR (400 MHz, CDCl₃) 8.24 (1H, s), 7.88 (1H, s), 7.56-7.54 (1H, m), 7.42-7.36 (1H, m), 7.15 (1H, dd, J=2.3, 8.9 Hz), 4.59-4.51 (2H, t, J=11.6 Hz), 4.50-4.45 (2H, t, J=5.4 Hz), 3.82 (2H, t, J=11.7 Hz), 3.68 (2H, t, J=5.2 Hz), 2.37-2.30 (2H, m) ppm.

### Example 112: (12S)-12-methyl-11,14-dioxa-4,5,19,20-tetraazatetracyclo[13.5.2.1^{2,5}.0^{18,21}] tricosa-1(20),2(23),3,15(22),16,18(21)-hexaene

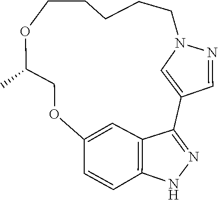 [SMILES: C[C@H]1COc2ccc3[nH]nc(c3c2)-c2cnn(c2)CCCCCO1]

Example 112 is prepared according to the synthesis route described in general scheme D, following the reaction procedures described for example 102, using (S)-(+)-1-benzyloxy-2-propanol, methyl 5-bromopentanoate, 4-bromo-1H-pyrazole and tert-butyl-dimethyl-[1-tetrahydropyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=327.1, t_{R}=2.30 min

LCMS method G: [M+H]⁺=327.2, t_{R}=2.26 min

¹H NMR (400 MHz, CDCl₃) 7.99 (1H, s), 7.93 (1H, s), 7.75 (1H, d, J=1.7 Hz), 7.36 (1H, m), 7.12 (1H, dd, J=2.3, 8.9 Hz), 4.41-4.29 (3H, m), 4.20 (1H, dd, J=2.7, 13.1 Hz), 3.84-3.74 (2H, m), 3.38-3.33 (1H, m), 1.99-1.90 (2H, m), 1.79-1.54 (4H, m), 1.32-1.29 (3H, m) ppm.

### Example 113: (12R,13R)-12,13-dimethyl-8,11,14-trioxa-4,5,19,20-tetraazatetracyclo [13.5.2.1^{2,5}.0^{18,21}]tricosa-1(20),2(23),3,15(22),16,18(21)-hexaene

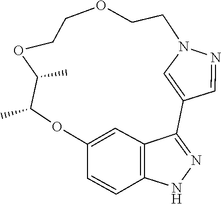 [SMILES: C[C@H]1OCCOCCn2cc(cn2)-c2n[nH]c3ccc(cc23)O[C@@H]1C]

Example 113 is prepared according to the synthesis route described in general scheme E, using 2-(2-benzyloxyethoxy)ethyl methanesulfonate intermediate 145, (2R,3R)-butane-2,3-diol and 1-tetrahydropyran-2-yl-3-[1-(2-trimethylsilylethoxymethyl)pyrazol-4-yl]indazol-5-ol intermediate 229 as starting materials.

LCMS method F: [M+H]⁺=343.3, t_{R}=2.08 min

LCMS method G: [M+H]⁺=343.3, t_{R}=2.08 min

¹H NMR (400 MHz, CDCl₃) 8.48 (1H, d, J=0.8 Hz), 8.03 (1H, d, J=0.5 Hz), 7.52 (1H, d, J=2.3 Hz), 7.42 (1H, dd, J=8.8, 0.42 Hz), 7.11 (1H, dd, J=2.3, 9.1 Hz), 4.55-4.44 (3H, m), 4.01-3.96 (2H, m), 3.90-3.82 (2H, m), 3.79-3.74 (1H, m), 3.64-3.60 (1H, m), 3.59-3.54 (1H, m), 1.39 (3H, d, J=6.4 Hz), 1.23 (3H, d, J=6.2 Hz) ppm.

### Example 114: (12S,13S)-12,13-dimethyl-8,11,14-trioxa-4,5,19,20-tetraazatetracyclo [13.5.2.1^{2,5}.0^{18,21}]tricosa-1(20),2(23),3,15(22),16,18(21)-hexaene

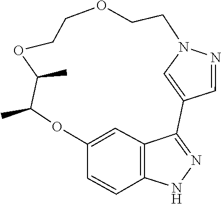 [SMILES: C[C@@H]1OCCOCCn2cc(cn2)-c2n[nH]c3ccc(cc23)O[C@H]1C]

Example 114 is prepared according to the synthesis route described in general scheme E using 2-(2-benzyloxyethoxy)ethyl methanesulfonate intermediate 145, (2S,3S)-butane-2,3-diol and 1-tetrahydropyran-2-yl-3-[1-(2-trimethylsilylethoxymethyl)pyrazol-4-yl]indazol-5-ol intermediate 229 as starting materials.

LCMS method F: [M+H]⁺=343.3, t_{R}=2.08 min

LCMS method G: [M+H]⁺=343.3, t_{R}=2.07 min

¹H NMR (400 MHz, CDCl₃) 8.46 (1H, s), 7.99 (1H, s), 7.52 (1H, d, J=2.5 Hz), 7.38 (1H, d, J=8.9 Hz), 7.08 (1H, dd, J=2.3, 8.7 Hz), 4.55-4.44 (3H, m), 4.01-3.96 (2H, m), 3.90-3.82 (2H, m), 3.78-3.74 (1H, m), 3.64-3.60 (1H, m), 3.54-3.48 (1H, m), 1.38 (3H, d, J=6.4 Hz), 1.23 (3H, d, J=6.5 Hz) ppm.

### Example 115: 8,13-dioxa-4,5,18,19,22-pentaazatetracyclo[12.5.2.1^{2,5}.0^{17,20}]docosa-1(19),2(22),3,14(21),15,17(20)-hexaene

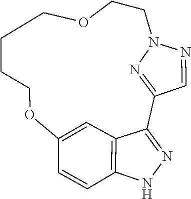 [SMILES: c1cc2[nH]nc3c2cc1OCCCCOCCn1ncc-3n1]

Example 115 is prepared according to the synthesis route described in general scheme D, using 4-benzyloxybutan-1-ol, 2-(4-bromotriazol-2-yl)ethanol intermediate 398 and tert-butyl-dimethyl-[1-tetrahydropyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=300.3, t_{R}=2.03 min

LCMS method G: [M+H]⁺=300.3, t_{R}=2.02 min

¹H NMR (400 MHz, CDCl₃) 8.32 (1H, d, J=2.3 Hz), 8.06 (1H, s), 7.38 (1H, dd, J=8.8, 0.6 Hz), 7.08 (1H, dd, J=2.6, 9.0 Hz), 4.78 (2H, m), 4.31 (2H, m), 4.08 (2H, m), 3.76 (2H, t, J=5.5 Hz), 2.25 (2H, m), 1.85 (2H, m) ppm.

### Example 116: (6R,10S)-6,10-dimethyl-9,13-dioxa-4,5,18,19-tetraazatetracyclo[12.5.2.1^{2,5}.0^{17,20}]docosa-1(19),2(22),3,14(21),15,17(20)-hexaene

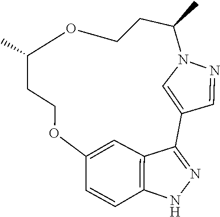 [SMILES: C[C@@H]1CCO[C@@H](C)CCOc2ccc3[nH]nc(c3c2)-c2cnn1c2]

Example 116 is prepared according to the synthesis route described in general scheme D, using 4-bromo-1H-pyrazole, ethyl (S)-3-hydroxybutanoate and tert-butyl-dimethyl-[1-tetrahydro pyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=327.2, t_{R}=2.25 min

LCMS method G: [M+H]⁺=327.2, t_{R}=2.27 min

¹H NMR (400 MHz, DMSO) 12.68 (1H, s), 8.55 (1H, s), 7.68-7.67 (1H, s), 7.46 (1H, dd, J=1.0, 2.0 Hz), 7.38 (1H, dd, J=0.6, 8.9 Hz), 6.93 (1H, dd, J=2.4, 9.0 Hz), 4.64-4.53 (1H, m), 4.36-4.29 (2H, m), 3.66-3.52 (2H, m), 3.42 (1H, d, J=22.4 Hz), 2.19 (1H, d, J=39.3 Hz), 2.13-2.09 (1H, m), 1.96-1.93 (2H, m), 1.56-1.53 (3H, m), 1.17-1.09 (3H, m) ppm.

### Example 117: (6R,8R)-6,8-dimethyl-9,13-dioxa-4,5,18,19-tetraazatetracyclo[12.5.2.1^{2,5}.0^{17,20}] docosa-1(19),2(22),3,14(21),15,17(20)-hexaene

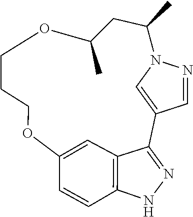 [SMILES: C[C@@H]1C[C@@H](C)n2cc(cn2)-c2n[nH]c3ccc(cc23)OCCCO1]

Example 117 is prepared according to the synthesis route described in general scheme E, using 1,3-propanediol, (2R,4S)-4-benzyloxypentan-2-ol and 1-tetrahydropyran-2-yl-3-[1-(2-trimethylsilylethoxymethyl)pyrazol-4-yl]indazol-5-ol intermediate 229 as starting materials.

LCMS method F: [M+H]⁺=327.3, t_{R}=2.20 min

LCMS method G: [M+H]⁺=327.3, t_{R}=2.20 min

¹H NMR (400 MHz, DMSO) 12.65 (1H, s), 8.58 (1H, s), 7.68 (1H, s), 7.55 (1H, d, J=2.1 Hz), 7.40-7.36 (1H, m), 6.93 (1H, dd, J=2.3, 8.9 Hz), 4.70-4.63 (1H, m), 4.38-4.21 (2H, m), 3.79-3.73 (1H, m), 3.64-3.58 (1H, m), 3.54-3.43 (1H, m), 2.44-2.36 (1H, m), 2.16-2.09 (2H, m), 1.99-1.92 (1H, m), 1.48 (3H, d, J=6.6 Hz), 1.13 (3H, d, J=6.3 Hz) ppm.

### Example 118: 7,7-difluoro-9,13-dioxa-4,5,18,19-tetraazatetracyclo[12.5.2.1^{2,5}.0^{17,20}] docosa-1(19),2(22),3,14(21),15,17(20)-hexaene

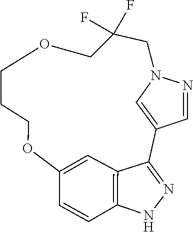 [SMILES: FC1(F)COCCCOc2ccc3[nH]nc(c3c2)-c2cnn(c2)C1]

Example 118 is prepared according to the synthesis route described in general scheme D, using 4-bromo-1H-pyrazole, 3-benzyloxypropyl 4-methylbenzenesulfonate intermediate 431, 2,2-difluoropropane-1,3-diol and tert-butyl-dimethyl-[1-tetrahydropyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=335.1, t_{R}=2.07 min

LCMS method G: [M+H]⁺=335.1, t_{R}=1.93 min

¹H NMR (400 MHz, DMSO) 12.79 (1H, s), 8.46 (1H, s), 7.75 (1H, s), 7.45-7.37 (2H, m), 6.98-6.94 (1H, dd, J=2.3, 8.8 Hz), 4.95-4.88 (2H, t, J=12.0 Hz), 4.31-4.25 (2H, t, J=7.8 Hz), 3.75-3.67 (4H, m), 2.12-2.04 (2H, m) ppm.

### Example 119: (13S)-4,13-dimethyl-7,11,14-trioxa-4,19,20,23-tetraazatetracyclo [13.5.2.1^{2,5}.0^{18,21}]tricosa-1(20),2,5(23),15(22),16,18(21)-hexaene

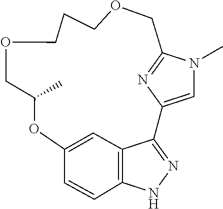 [SMILES: C[C@H]1COCCCOCc2nc(cn2C)-c2n[nH]c3ccc(cc23)O1]

Example 119 is prepared according to the synthesis route described in general scheme D, using 3-bromopropoxy-tert-butyl-dimethyl-silane, methyl (R)-(+)-lactate, methyl 4-bromo-1-methyl-imidazole-2-carboxylate, and tert-butyl-dimethyl-[1-tetrahydropyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=343.3, t_{R}=1.52 min

LCMS method G: [M+H]⁺=343.2, t_{R}=1.96 min

¹H NMR (400 MHz, CDCl₃) 9.81-9.48 (1H, m), 8.34 (1H, d, J=2.1 Hz), 7.34 (1H, s), 7.33 (1H, dd, J=0.7, 8.9 Hz), 7.09-7.06 (1H, m), 4.80 (1H, d, J=14.2 Hz), 4.69-4.65 (1H, m), 4.47 (1H, dd, J=4.6, 10.6 Hz), 4.38-4.30 (1H, m), 3.95-3.83 (2H, m), 3.74-3.73 (5H, m), 3.46-3.39 (1H, m), 2.09-2.05 (1H, m), 1.98-1.88 (1H, m), 1.43 (3H, d, J=6.5 Hz) ppm.

### Example 120: (12R)-7,7-difluoro-12-methyl-9,13-dioxa-4,5,18,19-tetraazatetracyclo [12.5.2.1^{2,5}.0^{17,20}]docosa-1(19),2(22),3,14(21),15,17(20)-hexaene

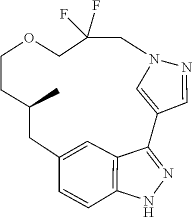 [SMILES: C[C@@H]1CCOCC(F)(F)Cn2cc(cn2)-c2n[nH]c3ccc(cc23)C1]

Example 120 is prepared according to the synthesis route described in general scheme D, using 4-bromo-1H-pyrazole, [(3S)-3-benzyloxybutyl] 4-methylbenzenesulfonate, 2,2-difluoropropane-1,3-diol and tert-butyl-dimethyl-[1-tetrahydropyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=349.3, t_{R}=3.05 min

LCMS method G: [M+H]⁺=349.3, t_{R}=3.03 min

¹H NMR (400 MHz, DMSO) 12.78 (s, 1H), 8.43 (s, 1H), 7.74 (s, 1H), 7.41 (d, J=8.1 Hz, 1H), 7.34 (s, 1H), 6.94 (dd, J=2.6, 8.9 Hz, 1H), 4.99 (dt, J=6.0, 15.9 Hz, 1H), 4.89-4.79 (m, 1H), 3.87-3.54 (m, 5H), 2.42 (t, J=13.0 Hz, 1H), 1.41 (d, J=5.2 Hz, 3H), 1.09 (t, J=5.8 Hz, 1H) ppm.

### Example 121: (8E)-11,14-dioxa-4,5,19,20-tetraazatetracyclo[13.5.2.1^{2,5}.0^{18,21}]tricosa-1(20),2(23),3,8,15(22),16,18(21)-heptaene

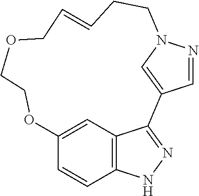 [SMILES: C1=C/COCCOc2ccc3[nH]nc(c3c2)-c2cnn(c2)CC/1]

Example 121 is prepared according to the synthesis route described below.

### Preparation of Intermediate 638: 2-allyloxyethyl methanesulfonate

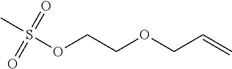

To a solution of 2-allyloxyethanol (2.09 mL, 19.60 mmol) in DCM (50 mL) at 0° C. was added triethylamine (9.54 mL, 68.60 mmol) and methanesulfonyl chloride (1.96 mL, 25.48 mmol). The reaction mixture was stirred at RT overnight. The reaction mixture was successively washed by saturated aqueous NaHCO₃ solution, saturated aqueous ammonium chloride solution and brine. The organic layer was dried over anhydrous sodium sulfate, filtered and evaporated under reduced pressure to afford 2-allyloxyethyl methanesulfonate 638 as a yellow oil which was used in the next step without further purification.

¹H NMR (400 MHz, CDCl₃) 5.95-5.85 (1H, m), 5.33-5.20 (2H, m), 4.40-4.38 (2H, m), 4.06-4.04 (2H, m), 3.73-3.71 (2H, m), 3.07-3.07 (3H, m) ppm.

### Preparation of Intermediate 639: [3-(1-allylpyrazol-4-yl)-1-tetrahydropyran-2-yl-indazol-5-yl]oxy-tert-butyl-dimethyl-silane

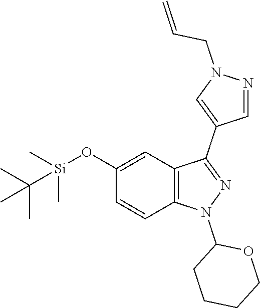 [SMILES: C=CCn1cc(-c2nn(C3CCCCO3)c3ccc(O[Si](C)(C)C(C)(C)C)cc23)cn1]

To a degassed solution of 1-allyl-4-bromo-pyrazole (467 mg, 2.5 mmol), tert-butyl-dimethyl-[1-tetrahydropyran-2-yl-3-(4,4,5,5-tetramethyl-1,3,2-dioxaborolan-2-yl)indazol-5-yl]oxy-silane (1.49 g, 3.25 mmol), potassium phosphate tribasic (1.59 g, 7.5 mmol), in dioxane (13.5 mL) and water (0.7 mL) was added tetrakis(triphenylphosphine)palladium(0) (144 mg, 0.125 mmol) and 2-dicyclohexylphosphino-2′,4′,6′-triisopropylbiphenyl (119 mg, 0.25 mmol). The reaction mixture was stirred at 110° C. overnight. The mixture was cooled to RT, diluted with water and extracted with ethyl acetate. The organic layer was washed with water, brine, dried over anhydrous sodium sulfate, filtered and evaporated under reduced pressure. The residue was purified by silica gel column chromatography using cyclohexane/ethyl acetate 100/0 to 80/20 as eluent to afford [3-(1-allylpyrazol-4-yl)-1-tetrahydropyran-2-yl-indazol-5-yl]oxy-tert-butyl-dimethyl-silane as a brown oil.

LCMS method F: [M+H]⁺=439.2, t_{R}=3.52 min

### Preparation of Intermediate 640: 3-(1-allylpyrazol-4-yl)-1-tetrahydropyran-2-yl-indazol-5-ol

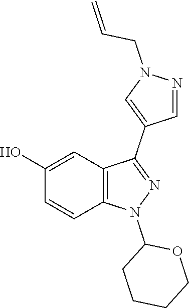 [SMILES: C=CCn1cc(-c2nn(C3CCCCO3)c3ccc(O)cc23)cn1]

To a solution of [3-(1-allylpyrazol-4-yl)-1-tetrahydropyran-2-yl-indazol-5-yl]oxy-tert-butyl-dimethyl-silane intermediate 639 (240 mg, 0.547 mmol) in THF (3 mL) was added TBAF (1M solution in THF) (711 μL, 0.711 mmol). The reaction mixture was stirred at RT for 2 h. The reaction mixture was quenched with saturated ammonium chloride solution and extracted with ethyl acetate. The combined organic layers were dried over anhydrous sodium sulfate, filtered and evaporated under reduced pressure. The residue was purified by silica gel column chromatography using cyclohexane/ethyl acetate 100/0 to 50/50 as eluent to afford 3-(1-allylpyrazol-4-yl)-1-tetrahydropyran-2-yl-indazol-5-ol as a white solid.

LCMS method F: [M+H]⁺=325.1, t_{R}=2.28 min

### Preparation of Intermediate 641: 5-(2-allyloxyethoxy)-3-(1-allylpyrazol-4-yl)-1-tetrahydropyran-2-yl-indazole

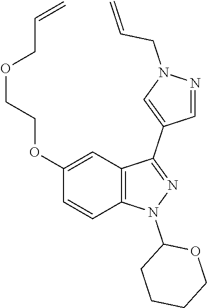 [SMILES: C=CCOCCOc1ccc2c(c1)c(-c1cnn(CC=C)c1)nn2C1CCCCO1]

To a solution of 3-(1-allylpyrazol-4-yl)-1-tetrahydropyran-2-yl-indazol-5-ol (100 mg, 0.308 mmol) in DMF (2 mL) was added cesium carbonate (150 mg, 0.462 mmol) and 2-allyloxyethyl methanesulfonate (61 mg, 0.339 mmol). The reaction mixture was stirred at 70° C. for 4 h. The reaction mixture was filtered then concentrated under reduced pressure. The crude product was dissolved in ethyl acetate and water was added. The phases were separated and the aqueous phase was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 50/50 as eluent to afford 5-(2-allyloxyethoxy)-3-(1-allylpyrazol-4-yl)-1-tetrahydropyran-2-yl-indazole as a brown oil.

LCMS method F: [M+H]⁺=409.4, t_{R}=2.78 min

### Preparation of Intermediate 642: (8E)-19-(oxan-2-yl)-11,14-dioxa-4,5,19,20-tetraaza tetracyclo[13.5.2.1^{2,5}.0^{18,21}]tricosa-1(20),2(23),3,8,15(22),16,18(21)-heptaene

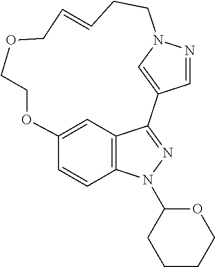 [SMILES: C1=C/COCCOc2ccc3c(c2)c(nn3C2CCCCO2)-c2cnn(c2)CC/1]

To a solution of 5-(2-allyloxyethoxy)-3-(1-allylpyrazol-4-yl)-1-tetrahydropyran-2-yl-indazole (120 mg, 0.293 mmol) in dry DCE (50 mL) at room temperature under argon was added Grubbs catalyst second Generation (37 mg, 0.044 mmol). The reaction mixture was stirred at 85° C. for 24 h. The reaction mixture was cooled to RT and concentrated under reduced pressure. The residue was purified by silica column chromatography using cyclohexane/ethyl acetate 100/0 to 40/60 as eluent to afford (8E)-19-(oxan-2-yl)-11,14-dioxa-4,5,19,20-tetraazatetracyclo [13.5.2.1^{2,5}.0^{18,21}]tricosa-1(20),2(23),3,8,15(22),16,18(21)-heptaene as a brown solid.

LCMS method F: [M+H]⁺=395.1, t_{R}=2.68 min

### Preparation of Example 121: (8E)-11,14-dioxa-4,5,19,20-tetraazatetracyclo [13.5.2.1^{2,5}.0^{18,21}] tricosa-1(20),2(23),3,8,15(22),16,18(21)-heptaene

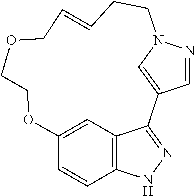 [SMILES: C1=C/COCCOc2ccc3[nH]nc(c3c2)-c2cnn(c2)CC/1]

To a solution of (8E)-19-(oxan-2-yl)-11,14-dioxa-4,5,19,20-tetraazatetracyclo [13.5.2.1^{2,5}.0^{18,21}]tricosa-1(20),2(23),3,8,15(22),16,18(21)-heptaene (50 mg, 0.126 mmol) in methanol (1.85 mL) and water (0.35 mL) was added p-toluenesulfonic acid monohydrate (120 mg, 0.663 mmol). The reaction mixture was stirred at 65° C. overnight. The reaction mixture was diluted with ethyl acetate and a saturated aqueous sodium hydrogen carbonate solution. After separation, the aqueous layer was extracted with ethyl acetate. The combined organic layers were washed with brine, dried over anhydrous sodium sulfate, filtered and evaporated under reduced pressure. The residue was purified by silica gel column chromatography using dichloromethane/methanol 100/0 to 90/10 as eluent. The appropriate fractions were evaporated under reduced pressure and the resulting product was triturated with cyclohexane, filtered and dried to afford (8E)-11,14-dioxa-4,5,19,20-tetraazatetracyclo[13.5.2.1^{2,5}.0^{18,21}]tricosa-1(20),2(23),3,8,15(22),16,18(21)-heptaene as an off-white solid.

LCMS method F: [M+H]⁺=311.1, t_{R}=2.10 min

LCMS method G: [M+H]⁺=311.2, t_{R}=1.89 min

¹H NMR (400 MHz, DMSO) 12.74 (1H, s), 8.13 (1H, s), 7.97 (1H, d, J=1.9 Hz), 7.73 (1H, s), 7.38-7.34 (1H, m), 6.99 (1H, dd, J=2.3, 8.9 Hz), 6.01-5.93 (1H, m), 5.71-5.63 (1H, m), 4.38-4.33 (2H, m), 4.32-4.29 (2H, m), 3.95 (2H, d, J=6.3 Hz), 3.77-3.73 (2H, m), 2.49-2.46 (2H, m) ppm.

### Example 122: (12S)-12-methyl-8,13-dioxa-4,5,18,19,22-pentaazatetracyclo[12.5.2.1^{2,5}.0^{17,20}]docosa-1(19),2(22),3,14(21),15,17(20)-hexaene

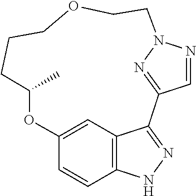 [SMILES: C[C@H]1CCCOCCn2ncc(n2)-c2n[nH]c3ccc(cc23)O1]

Example 122 is prepared according to the synthesis route described in general scheme D, using (2S)-5-oxotetrahydrofuran-2-carboxylic acid, 2-(4-bromotriazol-2-yl)ethanol intermediate 398 and tert-butyl-dimethyl-[1-tetrahydropyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=314.3, t_{R}=2.20 min

LCMS method G: [M+H]⁺=314.3, t_{R}=2.18 min

¹H NMR (400 MHz, CDCl₃) 8.26 (1H, d, J=2.5 Hz), 8.05 (1H, s), 7.37 (1H, d, J=8.8 Hz), 7.08 (1H, dd, J=2.5, 8.9 Hz), 4.80-4.76 (2H, m), 4.58-4.49 (1H, m), 4.17-4.11 (1H, m), 4.02-3.97 (1H, m), 3.92-3.86 (1H, m), 3.63-3.58 (1H, m), 2.82-2.74 (1H, m), 2.12-2.03 (1H, m), 1.77-1.67 (1H, m), 1.49-1.43 (1H, m), 1.46 (3H, d, J=6.1 Hz) ppm.

### Example 123: (6R,10R)-6,10-dimethyl-9,13-dioxa-4,5,18,19-tetraazatetracyclo[12.5.2.1^{2,5}.0^{17,20}]docosa-1(19),2(22),3,14(21),15,17(20)-hexaene

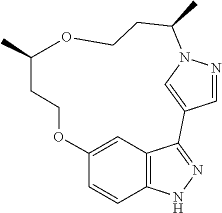 [SMILES: C[C@@H]1CCOc2ccc3[nH]nc(c3c2)-c2cnn(c2)[C@H](C)CCO1]

Example 123 is prepared according to the synthesis route described in general scheme E, using 1-tetrahydropyran-2-yl-3-[1-(2-trimethylsilylethoxymethyl)pyrazol-4-yl]indazol-5-ol intermediate 229, (2R)-4-trityloxybutan-2-ol intermediate 126 and [(3S)-3-benzyloxybutyl] 4-methylbenzene sulfonate as starting materials.

LCMS method F: [M+H]⁺=327.3, t_{R}=2.20 min

LCMS method G: [M+H]⁺=327.3, t_{R}=2.20 min

¹H NMR (400 MHz, DMSO) 12.69 (1H, s), 8.63 (1H, s), 7.66-7.65 (1H, m), 7.42-7.37 (2H, m), 6.93 (1H, dd, J=2.4, 8.8 Hz), 4.52-4.36 (2H, m), 4.32-4.23 (1H, m), 3.81-3.74 (1H, m), 3.62-3.51 (1H, m), 3.34-3.33 (1H, m), 2.34-2.21 (2H, m), 2.06-1.97 (1H, m), 1.89-1.80 (1H, m), 1.53-1.49 (3H, m), 1.19-1.16 (3H, m) ppm.

### Example 124: (12R)-12-methyl-8,13-dioxa-4,5,18,19,22-pentaazatetracyclo [12.5.2.1^{2,5}.0^{17,20}]docosa-1(19),2(22),3,14(21),15,17(20)-hexaene

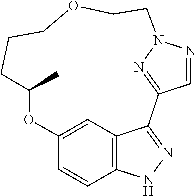 [SMILES: C[C@@H]1CCCOCCn2ncc(n2)-c2n[nH]c3ccc(cc23)O1]

Example 124 is prepared according to the synthesis route described in general scheme D, using (2R)-5-oxotetrahydrofuran-2-carboxylic acid, 2-(4-bromotriazol-2-yl)ethanol intermediate 398 and tert-butyl-dimethyl-[1-tetrahydropyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=314.2, t_{R}=2.24 min

LCMS method G: [M+H]⁺=314.3, t_{R}=2.25 min

¹H NMR (400 MHz, CDCl₃) 8.26 (1H, d, J=2.5 Hz), 8.09 (1H, s), 7.39 (1H, d, J=8.9 Hz), 7.09 (1H, dd, J=2.5, 8.9 Hz), 4.80-4.76 (2H, m), 4.58-4.49 (1H, m), 4.17-4.11 (1H, m), 4.03-3.97 (1H, m), 3.92-3.86 (1H, m), 3.63-3.58 (1H, m), 2.82-2.73 (1H, m), 2.10-2.03 (1H, m), 1.77-1.66 (1H, m), 1.50-1.41 (4H, m) ppm.

### Example 125: (12R)-12-methyl-9,13-dioxa-2,4,18,19,22-pentaazatetracyclo[12.5.2.1^{2,5}.0^{17,20}]docosa-1(19),3,5(22),14(21),15,17(20)-hexaene

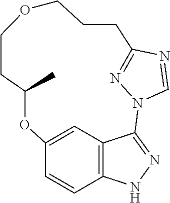 [SMILES: C[C@@H]1CCOCCCc2ncn(n2)-c2n[nH]c3ccc(cc23)O1]

Example 125 is prepared according to the synthesis route described below.

### Preparation of Intermediate 643: ethyl 3-(1-trityl-1,2,4-triazol-3-yl)propanoate

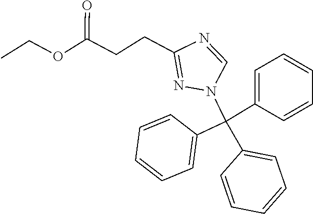 [SMILES: CCOC(=O)CCc1ncn(C(c2ccccc2)(c2ccccc2)c2ccccc2)n1]

To a solution of ethyl 3-(1H-1,2,4-triazol-3-yl)propanoate hydrochloride (1.5 g, 7.32 mmol) in DMF (30 mL) was added DIPEA (2.54 mL, 14.64 mmol) then trityl chloride (2.03 g, 7.32 mmol) and the reaction mixture was stirred at RT for 4 h. The reaction mixture was quenched with a saturated aqueous ammonium chloride solution and extracted with ethyl acetate. The combined organic layers were washed with brine, dried over anhydrous sodium sulfate, filtered and concentrated under reduced pressure to afford a mixture of two regioisomers ethyl 3-(1-trityl-1,2,4-triazol-3-yl)propanoate or ethyl 3-(4-trityl-1,2,4-triazol-3-yl)propanoate or ethyl 3-(2-trityl-1,2,4-triazol-3-yl)propanoate as a yellow viscous oil.

LCMS method F: [M+H]⁺=412.1, t_{R}=2.98 and 3.06 min

### Preparation of Intermediate 644: 3-(1-trityl-1,2,4-triazol-3-yl)propan-1-ol

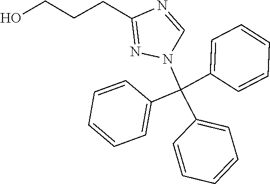 [SMILES: OCCCc1ncn(C(c2ccccc2)(c2ccccc2)c2ccccc2)n1]

To a mixture of two regioisomers ethyl 3-(1-trityl-1,2,4-triazol-3-yl)propanoate or ethyl 3-(4-trityl-1,2,4-triazol-3-yl)propanoate or ethyl 3-(2-trityl-1,2,4-triazol-3-yl)propanoate (2.82 g, 6.86 mmol) in THF (40 mL) under nitrogen atmosphere at 0° C. was added lithium aluminium hydride (1 M in THF) (8.9 mL, 8.9 mmol). The reaction mixture was allowed to warm up to RT and stirred for 1 h. The reaction mixture was slowly quenched at 0° C. with water, 15% Aqueous NaOH solution and water. The reaction mixture was filtered and the filtrate was evaporated under reduced pressure. The residue was purified by silica gel column chromatography using cyclohexane/ethyl acetate 100/0 to 0/100 as eluent. Just one regioisomer was recovered and evaporated to afford 3-(1-trityl-1,2,4-triazol-3-yl)propan-1-ol as a white solid.

LCMS method J: [M+H]⁺=370.1, t_{R}=3.84 min

### Preparation of Intermediate 645: 3-[3-[(3S)-3-benzyloxybutoxy]propyl]-1-trityl-1,2,4-triazole

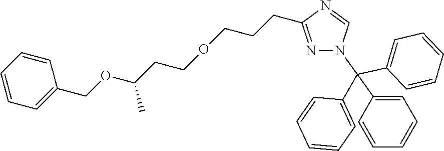 [SMILES: C[C@@H](CCOCCCc1ncn(C(c2ccccc2)(c2ccccc2)c2ccccc2)n1)OCc1ccccc1]

To a solution of 3-(1-trityl-1,2,4-triazol-3-yl)propan-1-ol (811 mg, 2.2 mmol) in dry DMF (8 mL) at 0° C. was added sodium hydride (60% dispersion in mineral oil) (132 mg, 3.3 mmol). The reaction mixture was stirred at RT for 30 min and a solution of [(3S)-3-benzyloxybutyl] 4-methylbenzenesulfonate intermediate 129 (883 mg, 2.64 mmol) in dry DMF (4 mL) was added. The reaction mixture was stirred at RT for 48 h. The reaction mixture was heated at 70° C. for 3 h then quenched with water and extracted with ethyl acetate. The combined organic layers were washed with brine, dried over anhydrous sodium sulfate, filtered and evaporated. The residue was purified by silica gel column chromatography using cyclohexane/ethyl acetate 100/0 to 70/30 as eluent to afford 3-[3-[(3S)-3-benzyloxybutoxy]propyl]-1-trityl-1,2,4-triazole as a colorless oil.

LCMS method F: [M+H]⁺=532.2, t_{R}=3.55 min

### Preparation of Intermediate 646: 3-[3-[(3S)-3-benzyloxybutoxy]propyl]-1H-1,2,4-triazole

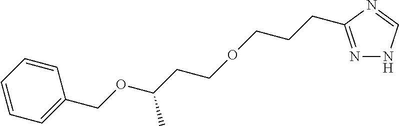 [SMILES: C[C@@H](CCOCCCc1nc[nH]n1)OCc1ccccc1]

To a solution of 3-[3-[(3S)-3-benzyloxybutoxy]propyl]-1-trityl-1,2,4-triazole (933 mg, 1.76 mmol) in methanol (9 mL) was added p-toluenesulfonic acid monohydrate (34 mg, 0.18 mmol) and the reaction mixture was stirred at RT overnight. The mixture was heated at 40° C. for 30 h. The mixture was diluted with ethyl acetate and a saturated aqueous NaHCO₃ solution. The layers were separated. The aqueous layer was extracted with ethyl acetate and the combined organic layers were washed with brine, dried over anhydrous sodium sulfate, filtered and evaporated under reduced pressure to give a crude mixture of 3-[3-[(3S)-3-benzyloxybutoxy]propyl]-1H-1,2,4-triazole and 3-[3-[(3S)-3-benzyloxybutoxy]propyl]-1-trityl-1,2,4-triazole as a colorless oil. The crude product was relaunched with the same conditions. To a mixture of 3-[3-[(3S)-3-benzyloxybutoxy]propyl]-1H-1,2,4-triazole and 3-[3-[(3S)-3-benzyloxybutoxy]propyl]-1-trityl-1,2,4-triazole (853 mg, 1.76 mmol) in methanol (8 mL) was added p-toluenesulfonic acid monohydrate (30 mg, 0.16 mmol) and the reaction mixture was heated at 40° C. overnight. The mixture was diluted with ethyl acetate and a saturated aqueous NaHCO₃ solution. The layers were separated. The aqueous layer was extracted with ethyl acetate and the combined organic layers were washed with brine, dried over anhydrous sodium sulfate, filtered and evaporated under reduced pressure. The residue was purified by silica gel column chromatography using dichloromethane/methanol 100/0 to 96/4 as eluent to afford 3-[3-[(3S)-3-benzyloxybutoxy]propyl]-1H-1,2,4-triazole as a colorless oil.

LCMS method F: [M+H]⁺=290.2, t_{R}=2.09 min

### Preparation of Intermediate 647: [3-[3-[3-[(3S)-3-benzyloxybutoxy]propyl]-1,2,4-triazol-1-yl]-1-tetrahydropyran-2-yl-indazol-5-yl]oxy-tert-butyl-dimethyl-silane

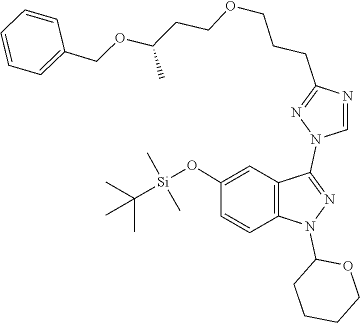 [SMILES: C[C@@H](CCOCCCc1ncn(-c2nn(C3CCCCO3)c3ccc(O[Si](C)(C)C(C)(C)C)cc23)n1)OCc1ccccc1]

To a solution of 3-[3-[(3S)-3-benzyloxybutoxy]propyl]-1H-1,2,4-triazole (282 mg, 0.98 mmol) in dichloromethane (3.7 mL) was added tert-butyl-dimethyl-[1-tetrahydropyran-2-yl-3-(4,4,5,5-tetramethyl-1,3,2-dioxaborolan-2-yl)indazol-5-yl]oxy-silane intermediate 61 (718 mg, 1.57 mmol), triethylamine (0.41 mL, 2.94 mmol) and Cu(OAc)₂ (71 mg, 0.39 mmol). The reaction mixture was stirred under oxygen atmosphere at 35° C. for 144 h. The reaction mixture was diluted with a saturated solution of ammonium chloride and extracted with dichloromethane. The combined organic layers were washed with a saturated aqueous NaHCO₃ solution and brine, dried over anhydrous sodium sulfate, filtered and evaporated under reduced pressure. The residue was purified by silica gel column chromatography using cyclohexane/ethyl acetate 100/0 to 80/20 as eluent to afford [3-[3-[3-[(3S)-3-benzyloxybutoxy]propyl]-1,2,4-triazol-1-yl]-1-tetrahydropyran-2-yl-indazol-5-yl]oxy-tert-butyl-dimethyl-silane as a yellow oil.

LCMS method F: [M+H]⁺=620.3, t_{R}=3.95 min

### Preparation of Intermediate 648: (2S)-4-[3-[1-[5-[tert-butyl(dimethyl)silyl]oxy-1-tetrahydropyran-2-yl-indazol-3-yl]-1,2,4-triazol-3-yl]propoxy]butan-2-ol

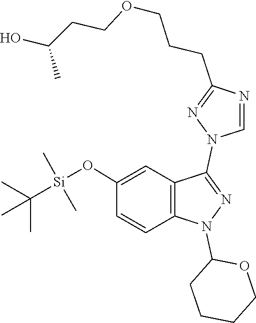 [SMILES: C[C@H](O)CCOCCCc1ncn(-c2nn(C3CCCCO3)c3ccc(O[Si](C)(C)C(C)(C)C)cc23)n1]

To a solution of [3-[3-[3-[(3S)-3-benzyloxybutoxy]propyl]-1,2,4-triazol-1-yl]-1-tetrahydro pyran-2-yl-indazol-5-yl]oxy-tert-butyl-dimethyl-silane (220 mg, 0.36 mmol) in ethyl acetate (8 mL) at RT was added palladium on carbon 10 wt % loading (22 mg). The reaction mixture was stirred under dihydrogen atmosphere at RT overnight. The reaction was heated at 50° C. for 6 h. The reaction mixture was filtered on a Celite pad and the filtrate was concentrated under reduced pressure. The residue was purified by silica gel column chromatography using cyclohexane/ethyl acetate 100/0 to 60/40 as eluent to afford (2S)-4-[3-[1-[5-[tert-butyl(dimethyl)silyl]oxy-1-tetrahydropyran-2-yl-indazol-3-yl]-1,2,4-triazol-3-yl] propoxy]butan-2-ol as a colorless oil.

LCMS method F: [M+H]⁺=530.3, t_{R}=3.40 min

### Preparation of Intermediate 649: [(1S)-3-[3-[1-[5-[tert-butyl(dimethyl)silyl]oxy-1-tetrahydropyran-2-yl-indazol-3-yl]-1,2,4-triazol-3-yl]propoxy]-1-methyl-propyl] methanesulfonate

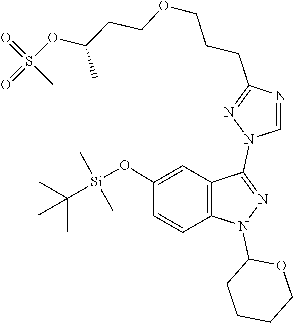 [SMILES: C[C@@H](CCOCCCc1ncn(-c2nn(C3CCCCO3)c3ccc(O[Si](C)(C)C(C)(C)C)cc23)n1)OS(C)(=O)=O]

To a solution of (2S)-4-[3-[1-[5-[tert-butyl(dimethyl)silyl]oxy-1-tetrahydropyran-2-yl-indazol-3-yl]-1,2,4-triazol-3-yl]propoxy]butan-2-ol (74 mg, 0.14 mmol) in dichloromethane (1.6 mL) was added triethylamine (39 μL, 0.28 mmol) and methanesulfonyl chloride (12 μL, 0.15 mmol). The reaction mixture was stirred at RT for 6 h. Additional triethylamine (20 μL, 0.14 mmol) and methanesulfonyl chloride (6 μL, 0.75 mmol) were added and the reaction mixture was stirred at RT overnight. The mixture was diluted with water and extracted with dichloromethane. The combined organic layers were washed with a saturated aqueous ammonium chloride solution and brine, dried over anhydrous sodium sulfate, filtered and evaporated under reduced pressure to afford [(1S)-3-[3-[1-[5-[tert-butyl(dimethyl)silyl]oxy-1-tetrahydropyran-2-yl-indazol-3-yl]-1,2,4-triazol-3-yl]propoxy]-1-methyl-propyl]methane sulfonate as a yellow viscous oil. The crude product was used in the next step without further purification.

LCMS method F: [M+H]⁺=608.2, t_{R}=3.52 min

### Preparation of Intermediate 650: (12R)-12-methyl-18-(oxan-2-yl)-9,13-dioxa-2,4,18,19,22-pentaazatetracyclo[12.5.2.1^{2,5}.0^{17,20}]docosa-1(19),3,5(22),14(21), 15,17(20)-hexaene

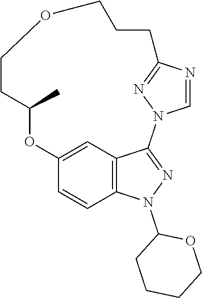 [SMILES: C[C@@H]1CCOCCCc2ncn(n2)-c2nn(C3CCCCO3)c3ccc(cc23)O1]

To a suspension of cesium carbonate (137 mg, 0.42 mmol) in anhydrous DMF (50 mL) at 85° C. was added dropwise during 1 h, [(1S)-3-[3-[1-[5-[tert-butyl(dimethyl)silyl]oxy-1-tetrahydro pyran-2-yl-indazol-3-yl]-1,2,4-triazol-3-yl]propoxy]-1-methyl-propyl] methanesulfonate (86 mg, 0.14 mmol) in DMF (50 mL). After addition the resulting reaction mixture was stirred at 85° C. overnight. The reaction mixture was concentrated under reduced pressure then water and ethyl acetate were added. After separation, the aqueous layer was extracted with ethyl acetate. The combined organic layers were washed with brine, dried over anhydrous sodium sulfate, filtered and evaporated under reduced pressure. The residue was purified by preparative TLC using dichloromethane/methanol 95/5 as eluent to afford (12R)-12-methyl-18-(oxan-2-yl)-9,13-dioxa-2,4,18,19,22-pentaazatetracyclo[12.5.2.1^{2,5}.0^{17,20}]docosa-1(19),3,5(22),14(21), 15,17(20)-hexaene as a light orange solid.

LCMS method F: [M+H]⁺=398.1, t_{R}=2.85 min

### Preparation of Example 125: (12R)-12-methyl-9,13-dioxa-2,4,18,19,22-pentaaza tetracyclo[12.5.2.1^{2,5}.0^{17,20}]docosa-1(19),3,5(22),14(21),15,17(20)-hexaene

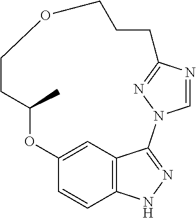 [SMILES: C[C@@H]1CCOCCCc2ncn(n2)-c2n[nH]c3ccc(cc23)O1]

To a solution of (12R)-12-methyl-18-(oxan-2-yl)-9,13-dioxa-2,4,18,19,22-pentaazatetracyclo [12.5.2.1^{2,5}.0^{17,20}]docosa-1(19),3,5(22),14(21),15,17(20)-hexaene (27 mg, 0.068 mmol) in methanol (1.1 mL) and water (0.2 mL) was added p-toluenesulfonic acid monohydrate (65 mg, 0.34 mmol). The reaction mixture was stirred at 65° C. overnight. Additional p-toluenesulfonic acid monohydrate (65 mg, 0.34 mmol) was added and the reaction mixture was heated at 65° C. for 22 h. The reaction mixture was diluted with dichloromethane and with a saturated aqueous NaHCO₃ solution. After separation, the aqueous layer was extracted with dichloromethane. The combined organic layers were washed with brine, dried over anhydrous sodium sulfate, filtered and evaporated under reduced pressure. The residue was purified by preparative TLC using dichloromethane/(methanol/ammonia 95/5) 90/10 as eluent to afford a mixture with the two regioisomers. The mixture was triturated in diisopropyl ether, filtered and rinsed with diisopropyl ether, to afford only the right regioisomer (12R)-12-methyl-9,13-dioxa-2,4,18,19,22-pentaazatetracyclo[12.5.2.1^{2,5}.0^{17,20}]docosa-1(19),3,5(22),14(21),15,17(20)-hexaene as a cream solid.

LCMS method F: [M+H]⁺=314.3, t_{R}=2.13 min

LCMS method G: [M+H]⁺=314.3, t_{R}=2.12 min

¹H NMR (400 MHz, CDCl₃) 9.61 (1H, d, J=0.8 Hz), 8.76 (1H, s), 8.17 (1H, d, J=2.3 Hz), 7.35 (1H, dd, J=9, 0.5 Hz), 7.13 (1H, dd, J=2.5, 9.1 Hz), 4.86-4.78 (1H, m), 4.23-4.17 (1H, m), 3.83-3.76 (1H, m), 3.67-3.63 (1H, m), 3.59-3.53 (1H, m), 3.18-3.10 (1H, m), 2.85-2.76 (1H, m), 2.65-2.56 (1H, m), 2.50-2.40 (1H, m), 2.35-2.26 (1H, m), 1.51-1.43 (1H, m), 1.48 (3H, d, J=6 Hz) ppm.

### Example 126: (10R,12R)-10,12-dimethyl-9,13-dioxa-4,5,18,19-tetraazatetracyclo [12.5.2.1^{2,5}.0^{17,20}]docosa-1(19),2(22),3,14(21),15,17(20)-hexaene

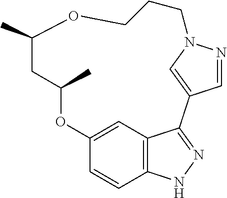 [SMILES: C[C@@H]1C[C@@H](C)Oc2ccc3[nH]nc(c3c2)-c2cnn(c2)CCCO1]

Example 126 is prepared according to the synthesis route described in general scheme E, using (2S,4S)-pentane-2,4-diol, 1,3-propanediol and 1-tetrahydropyran-2-yl-3-[1-(2-trimethylsilylethoxymethyl)pyrazol-4-yl]indazol-5-ol intermediate 229 as starting materials.

LCMS method F: [M+H]⁺=327.3, t_{R}=2.21 min

LCMS method G: [M+H]⁺=327.3, t_{R}=2.20 min

¹H NMR (400 MHz, DMSO) 12.71 (1H, s), 8.58 (1H, s), 7.65 (1H, s), 7.38-7.35 (1H, m), 7.29 (1H, d, J=1.9 Hz), 6.92 (1H, dd, J=2.5, 8.9 Hz), 4.69 (1H, q, J=5.9 Hz), 4.42-4.35 (1H, m), 4.22-4.14 (1H, m), 3.82-3.76 (1H, m), 3.68-3.60 (1H, m), 2.96-2.90 (1H, m), 2.32-2.27 (2H, m), 2.15-2.09 (1H, m), 1.65-1.58 (1H, m), 1.38-1.35 (3H, m), 1.18-1.15 (3H, m) ppm.

### Example 127: (8R,12R)-8,12-dimethyl-9,13-dioxa-4,5,18,19,22-pentaazatetracyclo [12.5.2.1^{2,5}.0^{17,20}]docosa-1(19),2(22),3,14(21),15,17(20)-hexaene

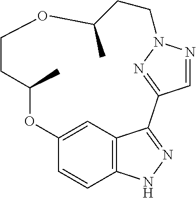 [SMILES: C[C@@H]1CCn2ncc(n2)-c2n[nH]c3ccc(cc23)O[C@H](C)CCO1]

Example 127 is prepared according to the synthesis route described in general scheme E, using methyl (3S)-3-hydroxybutanoate, (3R)-3-tetrahydropyran-2-yloxybutan-1-ol intermediate 236, 4,5-dibromo-2H-triazole and tert-butyl-dimethyl-[1-tetrahydropyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=328.3, t_{R}=2.36 min

LCMS method G: [M+H]⁺=328.3, t_{R}=2.33 min

¹H NMR (400 MHz, DMSO) 12.98 (1H, s), 8.25 (1H, d, J=2.5 Hz), 8.05-8.04 (1H, m), 7.43-7.40 (1H, m), 6.95 (1H, dd, J=2.5, 8.9 Hz), 4.70-4.47 (3H, m), 4.03-3.97 (1H, m), 3.85-3.80 (1H, m), 3.34-3.27 (1H, m), 2.49-2.38 (1H, m), 2.33-2.23 (2H, m), 1.60-1.52 (1H, m), 1.40-1.37 (3H, m), 1.18-1.15 (3H, m) ppm.

### Example 128: (6R,12R)-6,12-dimethyl-8,13-dioxa-4,5,18,19-tetraazatetracyclo[12.5.2.1^{2,5}.0^{17,20}]docosa-1(19),2(22),3,14(21),15,17(20)-hexaene

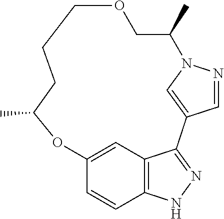 [SMILES: C[C@@H]1CCCOC[C@@H](C)n2cc(cn2)-c2n[nH]c3ccc(cc23)O1]

Example 128 is prepared according to the synthesis route described in general scheme E, using (2S)-2-benzyloxypropan-1-ol intermediate 66, (2R)-5-oxotetrahydrofuran-2-carboxylic acid and 1-tetrahydropyran-2-yl-3-[1-(2-trimethylsilylethoxymethyl)pyrazol-4-yl]indazol-5-ol intermediate 229 as starting materials.

LCMS method F: [M+H]⁺=327.3, t_{R}=2.28 min

LCMS method G: [M+H]⁺=327.3, t_{R}=2.27 min

¹H NMR (400 MHz, CDCl₃) 8.37 (1H, d, J=0.6 Hz), 7.89 (1H, d, J=0.8 Hz), 7.40 (1H, d, J=9.5 Hz), 7.27 (1H, d, J=2.4 Hz), 7.08 (1H, dd, J=2.4, 9.0 Hz), 4.70-4.65 (1H, m), 4.38-4.32 (1H, m), 3.77-3.68 (2H, m), 3.57-3.46 (2H, m), 2.69-2.64 (1H, m), 2.09-2.02 (1H, m), 1.75 (3H, d, J=6.9 Hz), 1.72-1.65 (1H, m), 1.53-1.45 (1H, m), 1.43 (3H, d, J=6 Hz) ppm.

### Example 129: (13S)-13-methyl-9,12,15-trioxa-4,5,20,21-tetraazatetracyclo[14.5.2.1^{2,5}.0^{19,22}]tetracosa-1(21),2(24),3,16(23),17,19(22)-hexaene

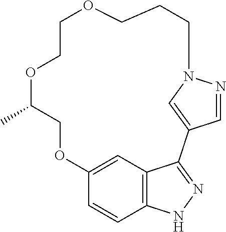 [SMILES: C[C@H]1COc2ccc3[nH]nc(c3c2)-c2cnn(c2)CCCOCCO1]

Example 129 is prepared according to the synthesis route described in general scheme E, using ethylene glycol, 3-bromopropoxymethylbenzene, (2S)-1-trityloxypropan-2-ol intermediate 102 and 1-tetrahydropyran-2-yl-3-[1-(2-trimethylsilylethoxymethyl)pyrazol-4-yl]indazol-5-ol intermediate 229 as starting materials.

LCMS method F: [M+H]⁺=343.3, t_{R}=2.01 min

LCMS method G: [M+H]⁺=343.3, t_{R}=2.00 min

¹H NMR (400 MHz, CDCl₃) 10.88-10.05 (1H, brs), 8.33-8.31 (1H, m), 8.16 (1H, s), 8.04 (1H, d, J=2.1 Hz), 7.32 (1H, d, J=8.9 Hz), 7.08 (1H, dd, J=2.3, 8.9 Hz), 4.54-4.41 (2H, m), 4.31-4.28 (2H, m), 4.00-3.90 (2H, m), 3.59-3.51 (3H, m), 3.31-3.24 (1H, m), 3.04-2.96 (1H, m), 2.17-2.09 (2H, m), 1.33-1.30 (3H, m) ppm.

### Example 130: (12R)-12-methyl-10,13-dioxa-4,5,18,19,22-pentaazatetracyclo[12.5.2.1^{2,5}.0^{17,20}] docosa-1(19),2(22),3,14(21),15,17(20)-hexaene

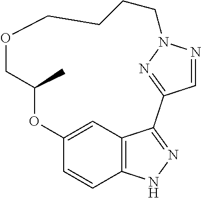 [SMILES: C[C@@H]1COCCCCn2ncc(n2)-c2n[nH]c3ccc(cc23)O1]

Example 130 is prepared according to the synthesis route described in general scheme D, using (2S)-2-(benzyloxy)propan-1-ol intermediate 66, 4,5-dibromo-2H-triazole, 2-(4-bromobutoxy)oxane and tert-butyl-dimethyl-[1-tetrahydropyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=314.3, t_{R}=2.31 min

LCMS method G: [M+H]⁺=314.3, t_{R}=2.29 min

¹H NMR (400 MHz, DMSO) 13.02 (1H, s), 8.49 (1H, d, J=2.3 Hz), 8.10-8.09 (1H, m), 7.43-7.39 (1H, m), 6.99 (1H, dd, J=2.5, 8.9 Hz), 4.63 (1H, ddd, J=2.7, 5.4, 14.1 Hz), 4.49-4.39 (2H, m), 3.83 (1H, dd, J=5.2, 10.3 Hz), 3.72-3.64 (1H, m), 3.56-3.42 (2H, m), 2.71-2.61 (1H, m), 1.99-1.83 (2H, m), 1.66-1.57 (1H, m), 1.39-1.35 (3H, m) ppm.

### Example 131: (14R)-14-methyl-8,11,15-trioxa-4,5,20,21-tetraazatetracyclo[14.5.2.1^{2,5}.0^{19,22}]tetracosa-1(21),2(24),3,16(23),17,19(22)-hexaene

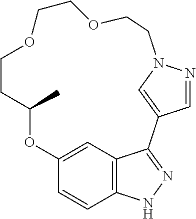 [SMILES: C[C@@H]1CCOCCOCCn2cc(cn2)-c2n[nH]c3ccc(cc23)O1]

Example 131 is prepared according to the synthesis route described in general scheme E, using 2-(2-benzyloxyethoxy)ethanol, methyl (3S)-3-hydroxybutanoate, 1-tetrahydropyran-2-yl-3-[1-(2-trimethylsilylethoxymethyl)pyrazol-4-yl]indazol-5-ol intermediate 229 as starting material.

LCMS method F: [M+H]⁺=343.3, t_{R}=2.11 min

LCMS method G: [M+H]⁺=343.3, t_{R}=2.10 min

¹H NMR (400 MHz, CDCl₃) 11.09-10.32 (1H, brs), 8.38-8.36 (1H, m), 8.10-8.08 (1H, m), 7.42 (1H, d, J=2.1 Hz), 7.34-7.30 (1H, m), 7.02 (1H, dd, J=2.1, 8.9 Hz), 4.84-4.76 (1H, m), 4.52-4.47 (2H, m), 3.96-3.59 (7H, m), 3.56-3.48 (1H, m), 2.46-2.36 (1H, m), 1.69-1.52 (1H, m), 1.42 (3H, d, J=6.1 Hz) ppm.

### Example 132: (6R,8S)-6,8-dimethyl-9,13-dioxa-4,5,18,19-tetraazatetracyclo[12.5.2.1^{2,5}.0^{17,20}]docosa-1(19),2(22),3,14(21),15,17(20)-hexaene

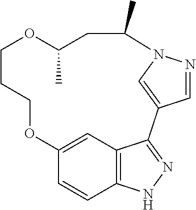 [SMILES: C[C@@H]1C[C@H](C)OCCCOc2ccc3[nH]nc(c3c2)-c2cnn1c2]

Example 132 is prepared according to the synthesis route described in general scheme E, using (2S,4S)-pentane-2,4-diol, 1,3-propanediol, 1-tetrahydropyran-2-yl-3-[1-(2-trimethylsilyl ethoxymethyl)pyrazol-4-yl]indazol-5-ol intermediate 229 as starting material.

LCMS method F: [M+H]⁺=327.3, t_{R}=2.12 min

LCMS method G: [M+H]⁺=327.3, t_{R}=2.10 min

¹H NMR (400 MHz, DMSO) 12.72 (1H, s), 8.54 (1H, s), 7.59 (1H, s), 7.40-7.37 (1H, m), 7.18 (1H, d, J=2.1 Hz), 6.94 (1H, dd, J=2.4, 9.0 Hz), 4.44 (2H, s), 4.24 (1H, dd, J=1.4, 6.4 Hz), 3.64-3.58 (1H, m), 3.41-3.35 (1H, m), 3.18-3.09 (1H, m), 2.48-2.35 (1H, m), 2.20-2.08 (1H, m), 1.91-1.84 (1H, m), 1.82-1.71 (1H, m), 1.53 (3H, d, J=6.8 Hz), 1.15-1.03 (3H, m) ppm.

### Example 133: (14R)-14-methyl-8,11,15-trioxa-4,5,20,21,24-pentaazatetracyclo[14.5.2.1^{2,5}.0^{19,22}]tetracosa-1(21),2(24),3,16(23),17,19(22)-hexaene

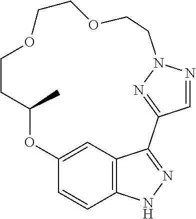 [SMILES: C[C@@H]1CCOCCOCCn2ncc(n2)-c2n[nH]c3ccc(cc23)O1]

Example 133 is prepared according to the synthesis route described in general scheme E, using 2-(2-benzyloxyethoxy)ethanol, methyl (3S)-3-hydroxybutanoate 4,5-dibromo-2H-triazole, 2-(4-bromobutoxy)oxane and tert-butyl-dimethyl-[1-tetrahydropyran-2-yl-3-(4,4,5,5-tetramethyl-1,3,2-dioxaborolan-2-yl)indazol-5-yl]oxy-silane intermediate 61 as starting materials.

LCMS method F: [M+H]⁺=344.3, t_{R}=2.10 min

LCMS method G: [M+H]⁺=344.3, t_{R}=2.08 min

¹H NMR (400 MHz, CDCl₃) 10.5-9.5 (1H, brs), 8.11 (1H, s), 7.95 (1H, d, J=2.3 Hz), 7.39-7.36 (1H, in), 7.08 (1H, dd, J=2.4, 9.0 Hz), 4.98-4.88 (2H, in), 4.70-4.64 (1H, in), 4.15-4.01 (3H, m), 3.75-3.65 (2H, m), 3.63-3.51 (3H, m), 2.29-2.20 (1H, m), 1.71-1.58 (1H, m), 1.41 (3H, d, J=6.3 Hz) ppm.

### Example 134: 7,14-dioxa-10,19,20-triazatetracyclo[13.5.2.1^{2,6}.0^{18,21}]tricosa-1(20),2(23), 3,5,15(22),16,18(21)-heptaen-9-one

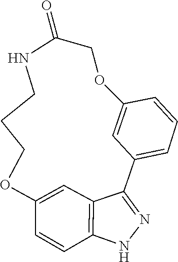 [SMILES: O=C1COc2cccc(c2)-c2n[nH]c3ccc(cc23)OCCCN1]

Example 134 is prepared according to the synthesis route described in general scheme I.

### Preparation of Intermediate 651: tert-butyl N-[3-(3-iodo-1-tetrahydropyran-2-yl-indazol-5-yl) oxypropyl]carbamate

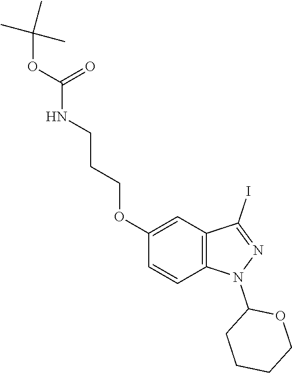 [SMILES: CC(C)(C)OC(=O)NCCCOc1ccc2c(c1)c(I)nn2C1CCCCO1]

A solution of 3-iodo-1-tetrahydropyran-2-yl-indazol-5-ol intermediate 16 (970 mg, 2.82 mmol), cesium carbonate (1.83 g, 5.55 mmol) and tert-butyl N-(3-bromopropyl)carbamate (2.01 g, 8.45 mmol) in DMF (11.6 mL) was heated at 50° C. for 10 h. Water was added and the reaction mixture was extracted with ethyl acetate, washed with water and brine, dried over anhydrous sodium sulfate, filtered and evaporated under reduced pressure. The residue was purified by silica gel column chromatography using cyclohexane/(ethyl acetate/ethanol 3/1) 100/0 to 60/40 as eluent to afford tert-butyl N-[3-(3-iodo-1-tetrahydropyran-2-yl-indazol-5-yl)oxypropyl]carbamate as a colorless oil.

LCMS method F: [M+H]⁺=502.1, t_{R}=3.20 min

### Preparation of Intermediate 652: tert-butyl N-[3-[3-(3-hydroxyphenyl)-1-tetrahydropyran-2-yl-indazol-5-yl]oxypropyl]carbamate

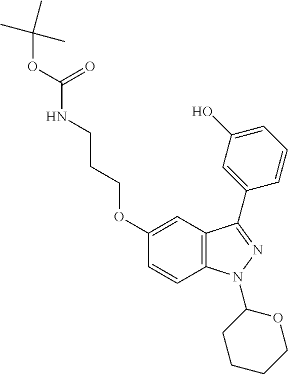 [SMILES: CC(C)(C)OC(=O)NCCCOc1ccc2c(c1)c(-c1cccc(O)c1)nn2C1CCCCO1]

To a degassed solution of tert-butyl N-[3-(3-iodo-1-tetrahydropyran-2-yl-indazol-5-yl)oxypropyl]carbamate (1.01 g, 2.02 mmol), 3-(4,4,5,5-tetramethyl-1,3,2-dioxaborolan-2-yl)phenol (667 mg, 3.03 mmol), tripotassium phosphate (1.28 g, 6.06 mmol) and XPhos (95 mg, 0.20 mmol) in dioxane (13 mL) and water (5.5 mL) was added tetrakis(triphenylphosphine)palladium(0) (116 mg, 0.10 mmol). The reaction mixture was stirred under microwave irradiations at 100° C. for 3 h. Additional 3-(4,4,5,5-tetramethyl-1,3,2-dioxaborolan-2-yl)phenol (222 mg, 1.01 mmol) and tetrakis(triphenylphosphine) palladium(0) (46 mg, 0.04 mmol) were added. The reaction mixture was stirred under microwave irradiations at 100° C. for 1 h. The reaction mixture was filtered through a Celite pad and washed with ethyl acetate. The filtrate was diluted with water and extracted with ethyl acetate. The combined organic layers were washed with water and brine, dried over anhydrous sodium sulfate and concentrated under reduced pressure. The residue was purified by silica gel column chromatography using dichloromethane/(ethyl acetate/ethanol 3/1) 70/30 as eluent to afford tert-butyl N-[3-[3-(3-hydroxyphenyl)-1-tetrahydropyran-2-yl-indazol-5-yl]oxypropyl] carbamate as an orange solid.

LCMS method F: [M+H]⁺=468.3, t_{R}=3.05 min

### Preparation of Intermediate 653: tert-butyl 2-[3-[5-[3-(tert-butoxycarbonyl amino)propoxy]-1-tetrahydropyran-2-yl-indazol-3-yl]phenoxy]acetate

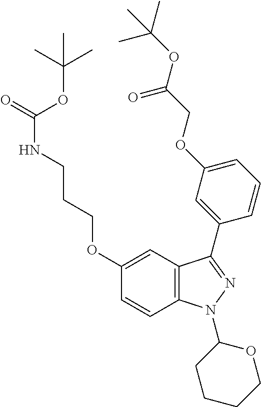 [SMILES: CC(C)(C)OC(=O)COc1cccc(-c2nn(C3CCCCO3)c3ccc(OCCCNC(=O)OC(C)(C)C)cc23)c1]

To a solution of tert-butyl N-[3-[3-(3-hydroxyphenyl)-1-tetrahydropyran-2-yl-indazol-5-yl]oxypropyl] carbamate (943 mg, 2.02 mmol) in acetonitrile (150 mL) at RT was added cesium carbonate (1.31 g, 4.04 mmol) and tert-butyl 2-bromoacetate (0.33 mL, 2.22 mmol). The reaction mixture was stirred at RT for 16 h. Additional tert-butyl 2-bromoacetate (0.15 mL, 1.01 mmol) was added. The reaction mixture was stirred at RT for 1 h. The reaction mixture was concentrated under reduced pressure. The residue was diluted with water and extracted with ethyl acetate. The combined organic layers were washed with water and brine, dried over anhydrous sodium sulfate and concentrated under reduced pressure. The residue was purified by silica gel column chromatography using cyclohexane/(ethyl acetate/ethanol 3/1) 100/0 to 60/40 as eluent to afford tert-butyl 2-[3-[5-[3-(tert-butoxycarbonylamino)propoxy]-1-tetrahydropyran-2-yl-indazol-3-yl]phenoxy]acetate as a pale yellow oil.

LCMS method F: [M+H]⁺=582.4, t_{R}=3.45 min

### Preparation of Intermediate 654: 2-[3-[5-(3-aminopropoxy)-1H-indazol-3-yl] phenoxy]acetic acid hydrochloride

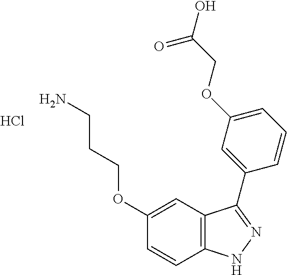 [SMILES: Cl ; NCCCOc1ccc2[nH]nc(-c3cccc(OCC(=O)O)c3)c2c1]

To a solution of tert-butyl 2-[3-[5-[3-(tert-butoxycarbonylamino)propoxy]-1-tetrahydropyran-2-yl-indazol-3-yl]phenoxy]acetate (1.11 g, 1.92 mmol) in dioxane (23 mL) was added 4M HCl in dioxane (9.6 mL, 38.4 mmol). The reaction mixture was heated at 60° C. for 16 h. The reaction mixture was evaporated to dryness under reduced pressure and the resulting solid was triturated with pentane, filtered and dried to afford 2-[3-[5-(3-aminopropoxy)-1H-indazol-3-yl]phenoxy]acetic acid hydrochloride as a cream solid.

The product was used in the next step without more purification.

LCMS method F: [M+H]⁺=342.2, t_{R}=1.43 min

### Preparation of Example 134: 7,14-dioxa-10,19,20-triazatetracyclo[13.5.2.1^{2,6}.0^{18,21}] tricosa-1(20),2(23),3,5,15(22),16,18(21)-heptaen-9-one

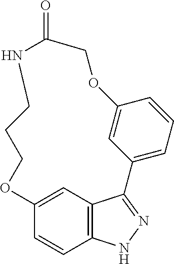 [SMILES: O=C1COc2cccc(c2)-c2n[nH]c3ccc(cc23)OCCCN1]

A solution of 2-[3-[5-(3-aminopropoxy)-1H-indazol-3-yl]phenoxy]acetic acid·hydrochloride (803 mg, 2.35 mmol) and DIPEA (3 mL) in DMF (400 mL) at RT was added dropwise over 1 h to a mixture of PyBOP (1.83 g, 3.53 mmol) in DMF (150 mL) and DIPEA (3 mL). The reaction mixture was stirred at RT for 16 h. The reaction mixture was evaporated under reduced pressure. The residue was diluted with dichloromethane and a saturated aqueous NaHCO₃ solution. The layers were separated and the aqueous layer was extracted with dichloromethane. The combined organic layers were washed with water and brine, dried over anhydrous sodium sulfate and evaporated under reduced pressure. The residue was purified by silica gel column chromatography using dichloromethane/methanol 98/2 to 92/8 as eluent. The resulting solid was solubilized in ethyl acetate and 1N aqueous HCl solution was added. The organic layer was washed with 1N aqueous HCl solution and then with a saturated aqueous NaHCO₃, water and brine. The organic layer was dried over anhydrous sodium sulfate, filtered and evaporated under reduced to afford 7,14-dioxa-10,19,20-triazatetracyclo[13.5.2.1^{2,6}.0^{18,21}]tricosa-1(20),2(23), 3,5,15(22),16,18(21)-heptaen-9-one as a white powder.

LCMS method F: [M+H]⁺=324.2, t_{R}=1.79 min

LCMS method G: [M+H]⁺=324.2, t_{R}=1.76 min

¹H NMR (400 MHz, MeOD) 7.64-7.62 (1H, m), 7.48-7.45 (3H, m), 7.42 (1H, t), 7.07-7.02 (2H, m), 4.78 (2H, s), 4.39 (2H, t), 3.41 (2H, t), 2.19-2.11 (2H, m), 1.31 (1H, s) ppm.

### Example 135: 7,14-dioxa-10,19,20-triazatetracyclo[13.5.2.1^{2,6}.0^{18,21}]tricosa-1(20),2(23),3, 5,15(22),16,18(21)-heptaene

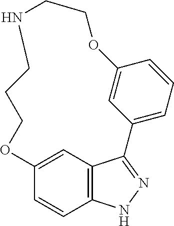 [SMILES: c1cc2cc(c1)-c1n[nH]c3ccc(cc13)OCCCNCCO2]

A solution of 7,14-dioxa-10,19,20-triazatetracyclo[13.5.2.1^{2,6}.0^{18,21}]tricosa-1(20),2(23),3,5, 15(22),16,18(21)-heptaen-9-one example 134 (41 mg, 0.127 mmol) with borane dimethylsulfide complex solution 2M in THF (4 mL) was stirred at RT for 16 h. The mixture was carefully quenched by adding methanol and stirred at RT for 4 h. The solvent was evaporated under reduced pressure. 4N HCl in dioxane was added and the mixture was stirred at RT for 2 h. The mixture was diluted with dichloromethane and a saturated aqueous NaHCO₃ solution. The phases were separated and the aqueous layer was extracted with dichloroethane. The combined organic layers were dried over anhydrous magnesium sulfate, filtered and evaporated under reduced pressure. The residue was purified by silica gel column chromatography using dichloromethane/methanol 100/0 to 95/5 as eluent The resulting oil was triturated with diisopropyl ether and concentrated under reduced pressure to afford 7,14-dioxa-10,19,20-triazatetracyclo[13.5.2.1^{2,6}.0^{18,21}]tricosa-1(20),2(23),3,5,15(22),16,18(21)-heptaene as a white solid.

LCMS method F: [M+H]⁺=310.3, t_{R}=1.29 min

LCMS method G: [M+H]⁺=310.2, t_{R}=2.46 min

¹H NMR (400 MHz, MeOD) 8.01 (2H, m), 7.56 (1H, d), 7.45 (1H, d), 7.37 (1H, t), 7.07-7.04 (1H, m), 6.94-6.91 (1H, m), 4.47-4.42 (4H, m), 3.10 (2H, t), 2.83 (2H, t), 2.08-2.03 (2H, m), 1.31 (1H, s) ppm.

The Examples compounds in Table 1A were made using a similar synthesis pathway as for Examples 134 and 135 starting from appropriate substrates, or according to the general scheme as indicated in Table 1.

TABLE 1A

| Example | IUPAC Name | |
|---|---|---|
| number | Structure | LCMS and NMR analysis |
| Example | 7,14-dioxa-4,10,19,20-tetraazatetracyclo[13.5.2.1^{2,6}.0^{18,21}]tricosa- | |
| 136 | 1(20),2,4,6(23),15,17,21-heptaen-9-one | |
| | 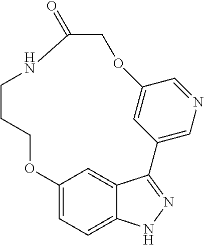 [SMILES: O=C1COc2cncc(c2)-c2n[nH]c3ccc(cc23)OCCCN1] | LCMS method E: [M + H]⁺ = 325.1, t_{R} = 1.60 min LCMS method D: [M + H]⁺ = 325.1, t_{R} = 2.67 min ¹H NMR (300 MHz, DMSO) 13.30 (s, 1H), 8.76 (s, 1H), 8.57 (s, 1H), 8.28 (s, 1H), 7.71 (s, 1H), 7.52 (d, J = 9.0 Hz, 1H), 7.31 (s, 1H), 7.01 (d, J = 9.0 Hz, 1H), 4.86 (s, 2H), 4.36 (m, 2H), 3.24 (m, 2H), 1.99 (m, 2H) ppm. |
| Example | 7,14-dioxa-4,10,19,20-tetraazatetracyclo[13.5.2.1^{2,6}.0^{18,21}]tricosa- | |
| 137 | 1(20),2,4,6(23),15,17,21-heptaene | |
| | 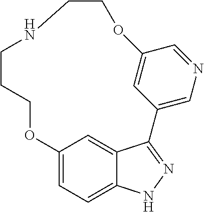 [SMILES: c1cc2[nH]nc3c2cc1OCCCNCCOc1cncc-3c1] | LCMS method E: [M + H]⁺ = 311.1, t_{R} = 1.18 min LCMS method D: [M + H]⁺ = 311.0, t_{R} = 0.87 min ¹H NMR (300 MHz, DMSO) 13.21 (s, 1H), 8.72 (s, 1H), 8.49 (s, 1H), 8.19 (s, 1H), 8.10 (s, 1H), 7.49 (d, J = 9.0 Hz, 1H), 7.00 (d, J = 8.9 Hz, 1H), 4.39 (m,, 4H), 2.97 (s, 2H), 2.63 (m, 2H), 1.93 (m, 2H) ppm. |
| Example | 5-fluoro-7,14-dioxa-10,19,20-triazatetracyclo[13.5.2.1^{2,6}.0^{18,21}]tricosa- | |
| 138 | 1(20),2,4,6(23),15,17,21-heptaen-9-one | |
| | 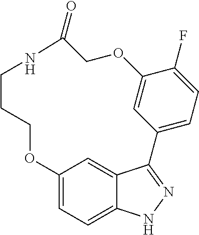 [SMILES: O=C1COc2cc(ccc2F)-c2n[nH]c3ccc(cc23)OCCCN1] | LCMS method E: [M + H]⁺ = 342.1, t_{R} = 2.32 min LCMS method D: [M + H]⁺ = 342.0, t_{R} = 2.63 min ¹H NMR (300 MHz, DMSO) 13.12 (s, 1H), 8.58 (t, J = 5.5 Hz, 1H), 7.61-7.54 (m, 1H), 7.48 (d, J = 9.16 Hz, 1H), 7.46 (d, J = 9.09 Hz, 1H), 7.35 (dd, J = 8.5 Hz, J = 11.38 Hz, 1H), 7.30 (s, 1H), 6.99 (d, J = 8.8 Hz, 1H), 4.87 (s, 2H), 4.35 (t, J = 8.30 Hz, 2H), 3.28-3.21 (m, 2H), 2.06-1.94 (m, 2H) ppm. |
| Example | 5-fluoro-7,14-dioxa-10,19,20-triazatetracyclo[13.5.2.1^{2,6}.0^{18,21}]tricosa- | |
| 139 | 1(20),2,4,6(23),15,17,21-heptaene | |
| | 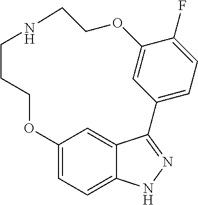 [SMILES: Fc1ccc2cc1OCCNCCCOc1ccc3[nH]nc-2c3c1] | LCMS method E: [M + H]⁺ = 328.1, t_{R} = 1.71 min LCMS method D: [M + H]⁺ = 328.1, t_{R} = 3.55 min ¹H NMR (300 MHz, DMSO) 13.04 (s, 1H), 8.21 (d, J = 7.7 Hz, 1H), 8.04 (s, 1H), 7.59-7.51 (m, 1H), 7.45 (d, J = 9.0 Hz, 1H), 7.29 (dd, J = 11.3, 8.6 Hz, 1H), 6.98 (d, J = 7.6 Hz, 1H), 4.43 (t, J = 5.2 Hz, 2H), 4.38 (t, J = 7.78 Hz, 2H), 3.02-2.94 (m, 2H), 2.71-2.62 (m, 2H), 2.00-1.87 (m, 2H) ppm. |
| Example | 5-methyl-7,14-dioxa-10,19,20-triazatetracyclo[13.5.2.1^{2,6}.0^{18,21}] tricosa- | |
| 140 | 1(20),2,4,6(23),15,17,21-heptaen-9-one | |
| | 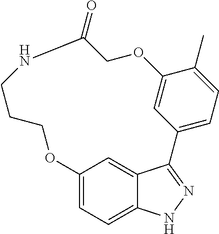 [SMILES: Cc1ccc2cc1OCC(=O)NCCCOc1ccc3[nH]nc-2c3c1] | LCMS method E: [M + H]⁺ = 338.1, t_{R} = 2.44 min ¹H NMR (300 MHz, DMSO) 13.04 (s, 1H), 8.57 (t, J = 5.5 Hz, 1H), 7.53 (d, J = 7.8 Hz, 1H), 7.46 (d, J = 9.0 Hz, 1H), 7.35 (s, 1H), 7.28 (s, 1H), 7.25 (d, J = 7.9 Hz, 1H), 6.97 (dd, J = 9.1, 1.1 Hz, 1H), 4.78 (s, 2H), 4.38-4.33 (m, 2H), 3.25 (m, 2H), 2.24 (s, 3H), 2.07-1.90 (brs, 2H) ppm. |
| Example | 5-methyl-7,14-dioxa-10,19,20-triazatetracyclo[13.5.2.1^{2,6}.0^{18,21}] tricosa- | |
| 141 | 1(20),2,4,6(23),15,17,21-heptaene | |
| | 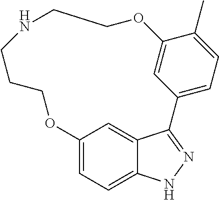 [SMILES: Cc1ccc2cc1OCCNCCCOc1ccc3[nH]nc-2c3c1] | LCMS method E: [M + H]⁺ = 324.1, t_{R} = 1.80 min ¹H NMR (300 MHz, DMSO) 12.98 (s, 1H), 8.01 (s, 1H), 7.96 (s, 1H), 7.48 (d, J = 7.8 Hz, 1H), 7.44 (d, J = 9.0 Hz, 1H), 7.21 (d, J = 7.8 Hz, 1H), 6.97 (d, J = 9.0 Hz, 1H), 4.46-4.30 (m, 4H), 3.07-2.91 (m, 2H), 2.75-2.61 (m, 2H), 2.19 (s, 3H), 2.01-1.82 (m, 2H) ppm. |
| Example | 4-chloro-7,14-dioxa-10,19,20-triazatetracyclo[13.5.2.1^{2,6}.0^{18,21}]tricosa- | |
| 142 | 1(20),2,4,6(23),15,17,21-heptaen-9-one | |
| | 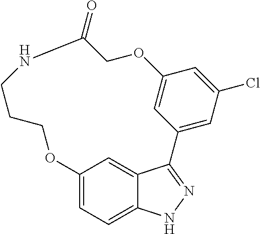 [SMILES: O=C1COc2cc(Cl)cc(c2)-c2n[nH]c3ccc(cc23)OCCCN1] | LCMS method B: [M + H]⁺ = 358.1, t_{R} = 2.66 min ¹H NMR (300 MHz, DMSO) 13.28 (s, 1H), 8.55 (t, J = 5.7 Hz, 1H), 7.60 (s, 1H), 7.50 (d, J = 9.0 Hz, 1H), 7.30 (d, J = 5.4 Hz, 2H), 7.02 (s, 1H), 7.00 (d, J = 7.1 Hz, 1H), 4.81 (s, 2H), 4.35 (t, J = 9.1 Hz, 2H), 3.28-3.18 (m, 2H), 2.16-1.76 (m, 2H) ppm. |
| Example | 4-chloro-7,14-dioxa-10,19,20-triazatetracyclo[13.5.2.1^{2,6}.0^{18,21}]tricosa- | |
| 143 | 1(20),2,4,6(23),15,17,21-heptaene | |
| | 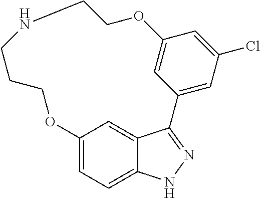 [SMILES: Clc1cc2cc(c1)-c1n[nH]c3ccc(cc13)OCCCNCCO2] | LCMS method E: [M + H]⁺ = 344.1, t_{R} = 1.99 min ¹H NMR (400 MHz, DMSO) 13.20 (s, 1H), 8.07 (d, J = 1.7 Hz, 1H), 8.02 (br s, 1H), 7.56 (dd, J = 1.7, 1.3 Hz, 1H), 7.47 (d, J = 9.0 Hz, 1H), 6.99 (dd, J = 9.0, 2.1 Hz, 1H), 6.92 (dd, J = 1.05, 2.1 Hz, 1H), 4.46-4.32 (m, 4H), 3.02-2.90 (m, 2H), 2.72-2.59 (m, 2H), 1.98-1.84 (m, 2H) ppm. |
| Example | 4-methyl-7,14-dioxa-10,19,20-triazatetracyclo[13.5.2.1^{2,6}.0^{18,21}]tricosa- | |
| 144 | 1(20),2(23),3,5,15,17,21-heptaen-9-one | |
| | 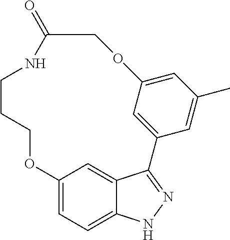 | LCMS method B: [M + H]⁺ = 338.1, t_{R} = 2.79 min LCMS method C: [M + H]⁺ = 338.1, t_{R} = 2.53 min ¹H NMR (300 MHz, DMSO) 13.10 (s, 1H), 8.55 (t, J = 5.40 Hz, 1H), 7.47 (d, J = 9.6 Hz, 2H), 7.34 (s, 1H), 7.14 (s, 1H), 6.98 (d, J = 9.6 Hz, 1H), 6.75 (s, 1H), 4.72 (s, 2H), 4.34 (t, J = 7.91 Hz, 2H), 3.27-3.17 (t, J = 5.41 Hz, 2H), 2.33 (s, 3H), 2.03-1.89 (m, J = 5.81 Hz,2H) ppm. |
| Example | 4-methyl-7,14-dioxa-10,19,20-triazatetracyclo[13.5.2.1^{2,6}.0^{18,21}]tricosa- | |
| 145 | 1(20),2(23),3,5,15,17,21-heptaene | |
| | 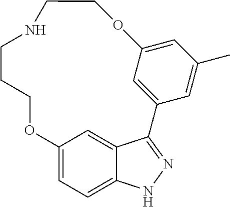 | LCMS method C: [M + H]⁺ = 324.2, t_{R} = 1.92 min LCMS method D: [M + H]⁺ = 324.2, t_{R} = 3.47 min ¹H NMR (400 MHz, DMSO) 13.02 (s, 1H), 8.00 (s, 1H), 7.80 (s, 1H), 7.44 (d, J = 9.0 Hz, 1H), 7.39 (s, 1H), 6.97 (dd, J = 9.0, 2.1 Hz, 1H), 6.68 (s, 1H), 4.38 (t, 2H), 4.33 (t, J = 5.9 Hz, 2H), 2.96 (t, J = 5.2 Hz, 2H), 2.67 (t, J = 3.5 Hz, 2H), 2.32 (s, 3H), 2.20 (s, 1H), 1.97-1.85 (m, 2H) ppm. |
| Example | 4-(oxan-4-yl)-7,14-dioxa-10,19,20-triazatetracyclo[13.5.2.1^{2,6}.0^{18,21}]tricosa- | |
| 146 | 1(20),2(23),3,5,15,17,21-heptaen-9-one | |
| | 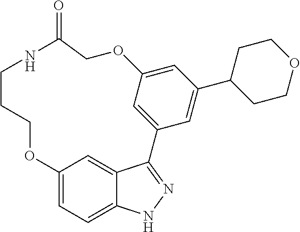 [SMILES: O=C1COc2cc(cc(C3CCOCC3)c2)-c2n[nH]c3ccc(cc23)OCCCN1] | LCMS method E: [M + H]⁺ = 408.2, t_{R} = 2.41 min LCMS method D: [M + H]⁺ = 408.2, t_{R} = 2.95 min ¹H NMR (300 MHz, DMSO) 13.10 (s, 1H), 8.56 (t, J = 5.4 Hz, 1H), 7.51 (s, 1H), 7.47 (d, J = 9.1 Hz, 1H), 7.35 (s, 1H), 7.18 (s, 1H), 6.98 (d, J = 9.1 Hz, 1H), 6.82 (s, 1H), 4.73 (s, 2H), 4.34 (t, J = 7.31Hz , 2H), 4.02-3.92 (m, 2H), 3.51-3.40 (m, 2H), 3.30-3.18 (m, 2H), 2.90-2.75 (m, 1H), 2.05-1.91 (m, 2H), 1.81- 1.63 (m, 4H) ppm. |
| Example | 4-(oxan-4-yl)-7,14-dioxa-10,19,20-triazatetracyclo[13.5.2.1^{2,6}.0^{18,21}]tricosa- | |
| 147 | 1(20),2(23),3,5,15,17,21-heptaene | |
| | 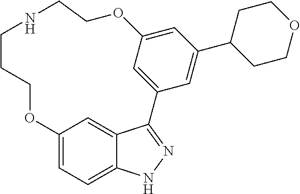 [SMILES: c1cc2[nH]nc3c2cc1OCCCNCCOc1cc-3cc(C2CCOCC2)c1] | LCMS method E: [M + H]⁺ = 394.2, t_{R} = 1.83 min LCMS method D: [M + H]⁺ = 394.2, t_{R} = 3.87 min ¹H NMR (400 MHz, DMSO) 13.03 (s, 1H), 8.02 (d, J = 1.7 Hz, 1H), 7.85 (s, 1H), 7.46 (s, 1H), 7.44 (d, J = 8.05 Hz, 1H), 6.97 (dd, J = 9.0, 2.1 Hz, 1H), 6.74 (t, J = 1.7 Hz, 1H), 4.42-4.37 (m, 2H), 4.37-4.32 (m, 2H), 4.00-3.91 (m, 2H), 3.44 (td, J = 11.3, 2.8 Hz, 2H), 3.02-2.91 (m, 2H), 2.79 (tt, J = 9.1, 4.4 Hz, 1H), 2.71-2.62 (m, 2H), 2.23 (br s, J = 25.9 Hz, 1H), 1.97-1.86 (m, 2H), 1.77-1.65 (m, 4H) ppm. |
| Example | 4-(oxan-3-yl)-7,14-dioxa-10,19,20-triazatetracyclo[13.5.2.1^{2,6}.0^{18,21}]tricosa- | |
| 148 | 1(20),2(23),3,5,15,17,21-heptaen-9-one | |
| | 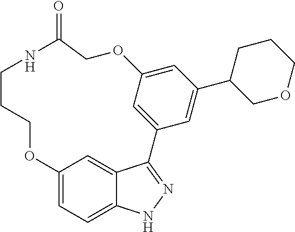 [SMILES: O=C1COc2cc(cc(C3CCCOC3)c2)-c2n[nH]c3ccc(cc23)OCCCN1] | LCMS method E: [M + H]⁺ = 408.1, t_{R} = 2.51 min ¹H NMR (400 MHz, DMSO) 13.11 (s, 1H), 8.56 (t, J = 6.0 Hz, 1H), 7.51 (s, 1H), 7.48 (d, J = 9.0 Hz, 1H), 7.35 (d, J = 1.6 Hz, 1H), 7.20 (brs, 1H), 6.99 (dd, J = 9.0, 2.1 Hz, 1H), 6.85 (t, J = 1.9 Hz, 1H), 4.74 (s, 2H), 4.40-4.28 (m, 2H), 3.93-3.82 (m, 2H), 3.46-3.36 (m, 2H), 3.24 (m, 2H), 2.88-2.78 (m, 1H), 2.04-1.93 (m, 3H), 1.85-1.73 (m, 1H), 1.72-1.63 (m, 2H) ppm. |
| Example | 4-(oxan-3-yl)-7,14-dioxa-10,19,20-triazatetracyclo[13.5.2.1^{2,6}.0^{18,21}]tricosa- | |
| 149 | 1(20),2(23),3,5,15,17,21-heptaene | |
| | 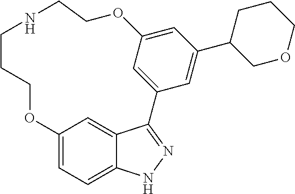 [SMILES: c1cc2[nH]nc3c2cc1OCCCNCCOc1cc-3cc(C2CCCOC2)c1] | LCMS method E: [M + H]⁺ = 394.1, t_{R} = 1.88 min LCMS method D: [M + H]⁺ = 394.2, t_{R} = 3.87 min ¹H NMR (400 MHz, DMSO) 13.03 (s, 1H), 8.02 (d, J = 1.4 Hz, 1H), 7.87 (t, J = 1.9 Hz, 1H), 7.46 (d, J = 3.4 Hz, 1H), 7.44 (d, J = 5.5 Hz, 1H), 6.97 (dd, J = 9.0, 2.1 Hz, 1H), 6.76 (t, J = 1.9 Hz, 1H), 4.39 (dd, J = 14.1, 6.6 Hz, 2H), 4.34 (t, J = 5.9 Hz, 2H), 3.91-3.82 (m, 2H), 3.44-3.35 (m, 2H), 2.96 (t, J = 5.8 Hz, 2H), 2.84-2.74 (m, 1H), 2.66 (dd, J = 6.2, 3.7 Hz, 2H), 2.26 (brs, 1H), 2.00-1.95 (m, 1H), 1.95-1.86 (m, 2H), 1.82-1.70 (m, 1H), 1.70-1.61 (m, 2H) ppm. |
| Example | (11S) or (11R)-11-(methoxymethyl)-7,14-dioxa-10,19,20-triazatetra | |
| 150 | cyclo[13.5.2.1^{2,6}.0^{18,21}]tricosa-1(20),2(23),3,5,15,17,21-heptaen-9-one | |
| | 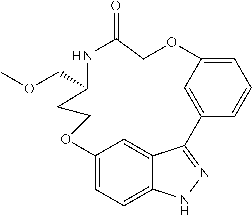 [SMILES: COC[C@@H]1CCOc2ccc3[nH]nc(c3c2)-c2cccc(c2)OCC(=O)N1] | Absolute stereochemistry unknown LCMS method E: [M + H]⁺ = 368.2, t_{R} = 2.37 min LCMS method D: [M + H]⁺ = 368.0, t_{R} = 2.80 min ¹H NMR (400 MHz, DMSO) 13.12 (s, 1H), 8.46 (d, J = 8.3 Hz, 1H), 7.57 (d, J = 7.7 Hz, 1H), 7.49 (d, J = 9.0 Hz, 1H), 7.38 (t, J = 7.9 Hz, 1H), 7.32 (d, J = 2.9 Hz, 2H), 7.00 (dd, J = 9.0, 2.1 H Hz, 1H), 6.93 (dd, J = 8.1, 1.7 Hz, 1H), 4.75 (s, 2H), 4.35 (t, J = 7.9 Hz, 2H), 4.21-4.08 (m, 1H), 3.47-3.35 (m, 2H), 3.30 (s, 3H), 2.18-2.06 (m, 1H), 1.90 (m, 1H) ppm. |
| Example | (11S) or (11R)-11-(methoxymethyl)-7,14-dioxa-10,19,20-triazatetracyclo | |
| 151 | [13.5.2.1^{2,6}.0^{18,21}]tricosa-1(20),2(23),3,5,15,17,21-heptaene | |
| | 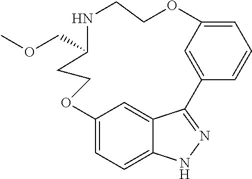 [SMILES: COC[C@@H]1CCOc2ccc3[nH]nc(c3c2)-c2cccc(c2)OCCN1] | Absolute stereochemistry unknown LCMS method E: [M + H]⁺ = 354.1, t_{R} = 1.94 min LCMS method D: [M + H]⁺ = 354.1, t_{R} = 3.50 min ¹H NMR (400 MHz, DMSO) 13.04 (s, 1H), 8.02 (s, 1H), 7.98 (s, 1H), 7.55 (d, J = 7.7 Hz, 1H), 7.46 (d, J = 9.0 Hz, 1H), 7.34 (t, J = 7.9 Hz, 1H), 6.97 (dd, J = 9.0, 2.1 Hz, 1H), 6.85 (dd, J = 8.1, 1.6 Hz, 1H), 4.52-4.37 (m, 3H), 4.36-4.26 (m, 1H), 3.36 (d, J = 5.5 Hz, 2H), 3.26 (s, 3H), 2.88-2.79 (m, 1H), 2.79-2.70 (m, 1H), 2.03-1.92 (m, 1H), 1.92-1.78 (m, 2H) ppm. |
| Example | (11R) or (11S)-11-(methoxymethyl)-7,14-dioxa-10,19,20-triazatetracyclo | |
| 152 | [13.5.2.1^{2,6}.0^{18,21}]tricosa-1(20),2(23),3,5,15,17,21-heptaen-9-one | |
| | 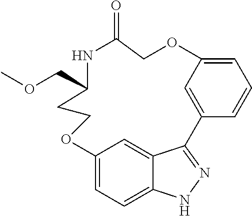 [SMILES: COC[C@H]1CCOc2ccc3[nH]nc(c3c2)-c2cccc(c2)OCC(=O)N1] | Absolute stereochemistry unknown LCMS method E: [M + H]⁺ = 368.1, t_{R} = 2.37 min ¹H NMR (400 MHz, DMSO) 13.14 (s, 1H), 8.47 (d, J = 8.3 Hz, 1H), 7.58 (d, J = 7.7 Hz, 1H), 7.50 (d, J = 8.9 Hz, 1H), 7.38 (t, J = 7.9 Hz, 1H), 7.34-7.30 (m, 2H), 6.99 (dd, J = 9.0, 2.1 Hz, 1H), 6.93 (dd, J = 8.2, 1.6 Hz, 1H), 4.76 (s, 2H), 4.35 (t, J = 7.9 Hz, 2H), 4.21-4.12 (m, 1H), 3.41 (m, 2H), 3.30 (s, 3H), 2.19- 2.06 (m, 1H), 1.97-1.84 (m, 1H) ppm. |
| Example | (11R) or (11S)-11-(methoxymethyl)-7,14-dioxa-10,19,20-triazatetracyclo | |
| 153 | [13.5.2.1^{2,6}.0^{18,21}]tricosa-1(20),2(23),3,5,15,17,21-heptaene | |
| | 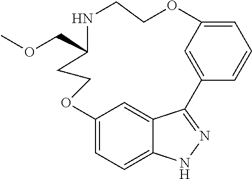 [SMILES: COC[C@H]1CCOc2ccc3[nH]nc(c3c2)-c2cccc(c2)OCCN1] | Absolute stereochemistry unknown LCMS method E: [M + H]⁺ = 354.1, t_{R} = 1.93 min LCMS method D: [M + H]⁺ = 354.1, t_{R} = 3.50 min ¹H NMR (400 MHz, DMSO) 13.04 (s, 1H), 8.02 (s, 1H), 7.98 (s, 1H), 7.55 (d, J = 7.7 Hz, 1H), 7.46 (d, J = 9.0 Hz, 1H), 7.34 (t, J = 7.9 Hz, 1H), 6.97 (dd, J = 9.0, 2.1 Hz, 1H), 6.85 (dd, J = 8.2, 1.6 Hz, 1H), 4.55-4.37 (m, 3H), 4.35-4.25 (m, 1H), 3.36 (dd, J = 5.4, 1.2 Hz, 2H), 3.26 (s, 3H), 2.89-2.79 (m, 1H), 2.75 (dd, J = 7.7, 5.5 Hz, 1H), 2.02-1.92 (m, 1H), 1.91-1.71 (m, 2H) ppm. |
| Example | 11-(fluoromethyl)-7,14-dioxa-10,19,20-triazatetracyclo [13.5.2.1^{2,6}.0^{18,21}]tricosa- | |
| 154 | 1(20),2,4,6(23),15,17,21-heptaen-9-one | |
| | 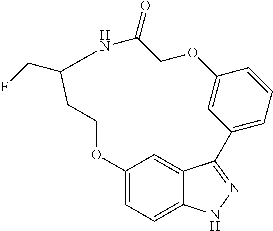 [SMILES: O=C1COc2cccc(c2)-c2n[nH]c3ccc(cc23)OCCC(CF)N1] | LCMS method E: [M + H]⁺ = 356.1, t_{R} = 2.40 min ¹H NMR (400 MHz, DMSO) 13.13 (s, 1H), 8.68 (d, J = 8.2 Hz, 1H), 7.57 (d, J = 7.7 Hz, 1H), 7.51 (d, J = 9.0 Hz, 1H), 7.39 (t, J = 7.9 Hz, 1H), 7.32 (s, 2H), 7.02 (dd, J = 9.0, 2.2 Hz, 1H), 6.95 (ddd, J = 8.2, 2.5, 0.9 Hz, 1H), 4.80 (s, 2H), 4.61-4.56 (m, 1H), 4.50-4.45 (m, 1H), 4.37 (t, J = 7.7 Hz, 2H), 4.32-4.20 (m, 1H), 2.20-2.06 (m, 1H), 2.02-1.87 (m, 1H) ppm. |
| Example | 11-(fluoromethyl)-7,14-dioxa-10,19,20-triazatetracyclo [13.5.2.1^{2,6}.0^{18,21}]tricosa- | |
| 155 | 1(20),2,4,6(23),15,17,21-heptaene | |
| | 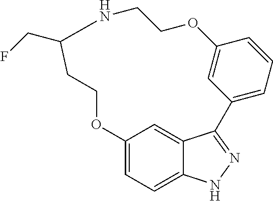 [SMILES: FCC1CCOc2ccc3[nH]nc(c3c2)-c2cccc(c2)OCCN1] | LCMS method E: [M + H]⁺ = 342.1, t_{R} = 1.82 min LCMS method D: [M + H]⁺ = 342.1, t_{R} = 3.56 min ¹H NMR (400 MHz, DMSO) 13.05 (s, 1H), 8.03 (d, J = 14.9 Hz, 1H), 7.56 (d, J = 7.7 Hz, 1H), 7.46 (d, J = 9.0 Hz, 1H), 7.34 (t, J = 7.9 Hz, 1H), 6.98 (dd, J = 9.0, 2.1 Hz, 1H), 6.85 (dd, J = 8.1, 1.6 Hz, 1H), 4.55-4.36 (m, 6H), 4.31 (ddd, J = 12.5, 5.7, 3.7 Hz, 1H), 3.41-3.33 (m, 1H), 2.93-2.77 (m, 2H), 2.28-2.17 (m, 1H), 1.95-1.76 (m, 2H) ppm. |
| Example | 7′,14′-dioxa-4′,10′,19′,20′-tetraazaspiro[cyclopropane-1,8′-tetracyclo | |
| 156 | [13.5.2.1^{2,6}.0^{18,21}]tricosane]-1′(20′),2′(23′),3′,5′,15′,17′,21′-heptaen-9′-one | |
| | 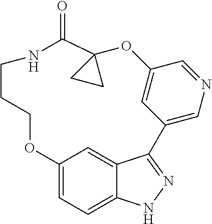 [SMILES: O=C1NCCCOc2ccc3[nH]nc(c3c2)-c2cncc(c2)OC12CC2] | LCMS method E: [M + H]⁺ = 351.1, t_{R} = 2.00 min LCMS method D: [M + H]⁺ = 351.1, t_{R} = 2.80 min ¹H NMR (400 MHz, DMSO) 13.27 (s, 1H), 8.69 (d, J = 1.6 Hz, 1H), 8.27 (t, J = 5.5 Hz, 1H), 8.23 (d, J = 2.7 Hz, 1H), 7.85 (dd, J = 2.6, 1.8 Hz, 1H), 7.52 (d, J = 9.0 Hz, 1H), 7.31 (d, J = 1.9 Hz, 1H), 7.00 (dd, J = 9.0, 2.2 Hz, 1H), 4.27 (t, J = 8.7 Hz, 2H), 3.24-3.13 (m, 2H), 2.05-1.93 (m, J = 8.5, 5.4 Hz, 2H), 1.57 (t, J = 6.4 Hz, 2H), 1.28-1.22 (m, 2H) ppm. |
| Example | 7′,14′-dioxa-4′,10′,19′,20′-tetraazaspiro[cyclopropane-1,8′-tetracyclo | |
| 157 | [13.5.2.1^{2,6}.0^{18,21}]tricosane]-1′(20′),2′(23′),3′,5′,15′,17′,21′-heptaene | |
| | 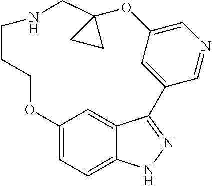 [SMILES: c1cc2[nH]nc3c2cc1OCCCNCC1(CC1)Oc1cncc-3c1] | LCMS method E: [M + H]⁺ = 337.1, t_{R} = 1.39 min LCMS method D: [M + H]⁺ = 337.1, t_{R} = 3.46 min ¹H NMR (400 MHz, DMSO) 13.21 (s, 1H), 8.74 (d, J = 1.6 Hz, 1H), 8.71-8.66 (m, 1H), 8.21 (d, J = 1.7 Hz, 1H), 8.08 (d, J = 2.6 Hz, 1H), 7.50 (d, J = 9.0 Hz, 1H), 7.01 (dd, J = 9.0, 2.1 Hz, 1H), 4.37 (t, J = 7.5 Hz, 2H), 3.11-3.00 (m, 2H), 2.59 (s, 2H), 2.33 (brs, J = 1.8 Hz, 1H), 1.97-1.86 (m, J = 5.0 Hz, 2H), 1.06 (q, J = 5.6 Hz, 2H), 0.89 (q, J = 5.4 Hz, 2H) ppm. |
| Example | 13-methyl-7,14-dioxa-4,10,19,20-tetraazatetracyclo[13.5.2.1^{2,6}.0^{18,21}]tricosa- | |
| 158 | 1(20),2,4,6(23),15,17,21-heptaen-9-one | |
| | 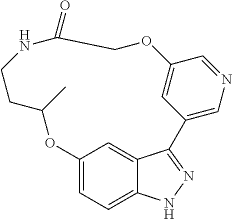 [SMILES: CC1CCNC(=O)COc2cncc(c2)-c2n[nH]c3ccc(cc23)O1] | LCMS method C: [M + H]⁺ = 339.1, t_{R} = 1.97 min LCMS method D: [M + H]⁺ = 339.0, t_{R} = 2.95 min ¹H NMR (300 MHz, DMSO) 13.29 (s, 1H), 8.75 (s, 1H), 8.60 (t, J = 5.9 Hz, 1H), 8.28 (d, J = 2.7 Hz, 1H), 7.68 (s, 1H), 7.51 (d, J = 9.0 Hz, 1H), 7.27 (s, 1H), 7.00 (d, 1H), 4.88 (s, 2H), 4.64-4.52 (m, J = 10.2, 6.4 Hz, 1H), 3.79-3.63 (m, 1H), 3.00- 2.83 (m, J = 13.5 Hz, 1H), 2.28 (t, J = 13.1 Hz, 1H), 1.54-1.45 (m, 1H), 1.43 (d, J = 5.9 Hz, 3H) ppm. |
| Example | 13-methyl-7,14-dioxa-4,10,19,20-tetraazatetracyclo[13.5.2.1^{2,6}.0^{18,21}]tricosa- | |
| 159 | 1(20),2,4,6(23),15,17,21-heptaene | |
| | 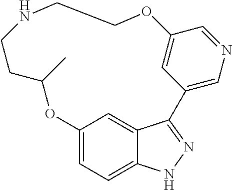 [SMILES: CC1CCNCCOc2cncc(c2)-c2n[nH]c3ccc(cc23)O1] | LCMS method E: [M + H]⁺ = 325.2, t_{R} = 1.43 min LCMS method D: [M + H]⁺ = 325.2, t_{R} = 3.55 min ¹H NMR (300 MHz, DMSO) 13.18 (s, 1H), 8.71 (s, 1H), 8.56 (s, 1H), 8.19 (d, J = 2.5 Hz, 1H), 8.13 (s, 1H), 7.48 (d, J = 9.0 Hz, 1H), 6.98 (dd, J = 9.0, 1.9 Hz, 1H), 4.69-4.56 (m, J = 13.5, 6.5 Hz, 1H), 4.50-4.32 (m, 2H), 3.06 (s, 1H), 2.91-2.79 (m, J = 11.4, 4.8 Hz, 2H), 2.15 (t, J = 12.1 Hz, 1H), 1.50 ( m, J = 13.3, 9.3, 4.4 Hz, 1H), 1.37 (d, J = 6.0 Hz, 3H) ppm. |
| Example | 8,10-dimethyl-7,14-dioxa-4,10,19,20-tetraazatetracyclo [13.5.2.1^{2,6}.0^{18,21}]tricosa- | |
| 160 | 1(20),2,4,6(23),15,17,21-heptaen-9-one | |
| | 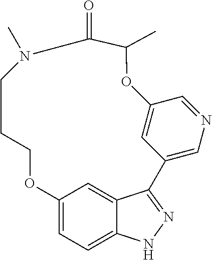 | LCMS method E: [M + H]⁺ = 353.2, t_{R} = 1.96 min LCMS method D: [M + H]⁺ = 353.2, t_{R} = 3.90 min ¹H NMR (300 MHz, DMSO) 13.25 (s, J = 36.1 Hz, 1H), 8.62 (s, J = 27.7 Hz, 1H), 8.25 (d, J = 2.7 Hz, 1H), 7.57 (t, J = 2.2 Hz, 1H), 7.51 (d, J = 9.0 Hz, 1H), 7.19 (d, J = 2.3 Hz, 1H), 7.01 (dd, J = 9.0, 2.1 Hz, 1H), 5.38 (q, J = 6.5 Hz, 1H), 4.44 (td, J = 11.8, 5.9 Hz, 1H), 4.27 (td, J = 12.1, 3.9 Hz, 1H), 4.09-4.00 (m, 1H), 3.15 (s, 3H), 2.76-2.67 (m, J = 8.5, 4.5 Hz, 2H), 1.86-1.72 (m, J = 15.5, 8.8 Hz, 1H), 1.56 (d, J = 6.6 Hz, 3H) ppm. |
| Example | 8,10-dimethyl-7,14-dioxa-4,10,19,20-tetraazatetracyclo [13.5.2.1^{2,6}.0^{18,21}]tricosa- | |
| 161 | 1(20),2,4,6(23),15,17,21-heptaene | |
| | 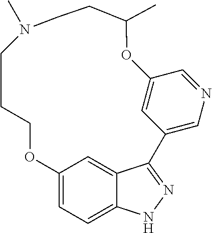 | LCMS method C: [M + H]⁺ = 339.1, t_{R} = 1.56 min LCMS method D: [M + H]⁺ = 339.2, t_{R} = 3.91 min ¹H NMR (300 MHz, DMSO) 13.25 (s, 1H), 8.78 (s, 1H), 8.47 (s, 1H), 8.17 (s, 1H), 7.90 (s, 1H), 7.52 (d, J = 9.0 Hz, 1H), 7.02 (d, J = 9.0, 2.0 Hz, 1H), 4.83 (q, J = 6.4, 3.2 Hz, 1H), 4.45-4.20 (m, 2H), 2.85 (dd, J = 13.8, 7.1 Hz, 1H), 2.65 (dd, J = 13.8, 3.0 Hz, 1H), 2.10 (s, 3H), 1.91-1.79 (m, J = 7.5, 4.4 Hz, 2H), 1.33 (d, J = 6.2 Hz, 3H) ppm. |
| Example | (13R)-13-methyl-7,14-dioxa-4,10,19,20-tetraazatetracyclo | |
| 162 | [13.5.2.1^{2,6}.0^{18,21}]tricosa-1(20),2,4,6(23),15(22),16,18(21)-heptaen-9-one | |
| | 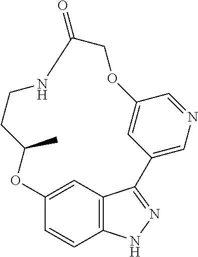 [SMILES: C[C@@H]1CCNC(=O)COc2cncc(c2)-c2n[nH]c3ccc(cc23)O1] | LCMS method E: [M + H]⁺ = 339.1, t_{R} = 1.81 min LCMS method D: [M + H]⁺ = 339.1, t_{R} = 2.63 min ¹H NMR (400 MHz, DMSO) 13.29 (s, 1H), 8.76 (d, J = 1.4 Hz, 1H), 8.61 (dd, J = 7.3, 4.8 Hz, 1H), 8.28 (t, J = 2.9 Hz, 1H), 7.69 (t, J = 2.2 Hz, 1H), 7.52 (d, J = 9.0 Hz, 1H), 7.28 (d, J = 2.2 Hz, 1H), 7.00 (dt, J = 5.8, 2.9 Hz, 1H), 4.85 (s, 2H), 4.65- 4.53 (m, 1H), 3.78-3.67 (m, 1H), 2.99-2.88 (m, 1H), 2.35-2.24 (m, 1H), 1.54-1.46 (m, 1H), 1.44 (d, J = 6.0 Hz, 3H) ppm. |
| Example | (13R)-13-methyl-7,14-dioxa-4,10,19,20-tetraazatetracyclo | |
| 163 | [13.5.2.1^{2,6}.0^{18,21}]tricosa-1(20),2,4,6(23),15(22),16,18(21)-heptaene | |
| | 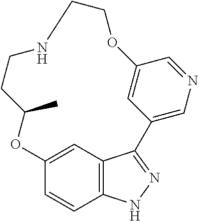 [SMILES: C[C@@H]1CCNCCOc2cncc(c2)-c2n[nH]c3ccc(cc23)O1] | LCMS method E: [M + H]⁺ = 325.1, t_{R} = 1.38 min LCMS method D: [M + H]⁺ = 325.1, t_{R} = 3.32 min ¹H NMR (400 MHz, DMSO) 13.18 (s, 1H), 8.71 (d, J = 1.5 Hz, 1H), 8.58-8.56 (m, 1H), 8.18 (d, J = 2.7 Hz, 1H), 8.14 (d, J = 1.6 Hz, 1H), 7.48 (d, J = 9.0 Hz, 1H), 6.98 (dd, J = 9.0, 2.1 Hz, 1H), 4.71-4.58 (m, 1H), 4.49-4.35 (m, 3H), 3.12-3.02 (m, 1H), 2.92-2.79 (m, 2H), 2.15 (t, J = 12.1 Hz, 1H), 1.49 (ddd, J = 13.3, 9.3, 4.4 Hz, 1H), 1.37 (d, J = 6.0 Hz, 3H) ppm. |
| Example | (7S,9S)-15-oxa-11,20,21-triazapentacyclo[14.5.2.1^{2,6}.0^{7,9}.0^{19,22}] tetracosa- | |
| 164 | 1(21),2(24),3,5,16(23),17,19(22)-heptaen-10-one | |
| | 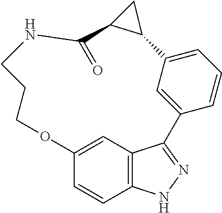 [SMILES: O=C1NCCCOc2ccc3[nH]nc(c3c2)-c2cccc(c2)[C@H]2C[C@H]12] | LCMS method F: [M + H]⁺ = 334.1, t_{R} = 2.04 min LCMS method G: [M + H]⁺ = 334.1, t_{R} = 1.96 min ¹H NMR (400 MHz, DMSO) 13.18 (s, 1H), 8.71 (d, J = 1.5 Hz, 1H), 8.58-8.56 (m, 1H), 8.18 (d, J = 2.7 Hz, 1H), 8.14 (d, J = 1.6 Hz, 1H), 7.48 (d, J = 9.0 Hz, 1H), 6.98 (dd, J = 9.0, 2.1 Hz, 1H), 4.71-4.58 (m, 1H), 4.49-4.35 (m, 3H), 3.12-3.02 (m, 1H), 2.92-2.79 (m, 2H), 2.15 (t, J = 12.1 Hz, 1H), 1.49 (ddd, J = 13.3, 9.3, 4.4 Hz, 1H), 1.37 (d, J = 6.0 Hz, 3H) ppm. |
| Example 165 | 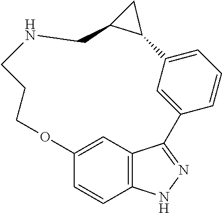 [SMILES: c1cc2cc(c1)[C@H]1C[C@@H]1CNCCCOc1ccc3[nH]nc-2c3c1] | LCMS method F: [M + H]⁺ = 320.1, t_{R} = 1.46 min LCMS method G: [M + H]⁺ = 320.2, t_{R} = 2.34 min ¹H NMR (400 MHz, DMSO) 13.01 (1H, s), 8.31 (1H, s), 7.66- 7.63 (2H, m), 7.48 (1H, d, J = 8.8 Hz), 7.36 (1H, t, J = 7.7 Hz), 7.05 (1H, d, J = 7.6 Hz), 6.98 (1H, dd, J = 2.4, 7.6 Hz), 4.36- 4.27 (2H, m), 3.08 (1H, dd, J = 3.9, 11.7 Hz), 2.76-2.67 (3H, m), 2.15-1.97 (4H, m), 1.03-0.95 (2H, m) ppm. |
| Example | 14-oxa-10,19,20-triazatetracyclo[13.5.2.1^{2,6}.0^{18,21}]tricosa- | |
| 166 | 1(20),2(23),3,5,15(22),16,18(21)-heptaen-9-one | |
| | 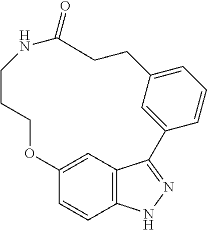 [SMILES: O=C1CCc2cccc(c2)-c2n[nH]c3ccc(cc23)OCCCN1] | LCMS method F: [M + H]⁺ = 322.1, t_{R} = 1.93 min LCMS method G: [M + H]⁺ = 322.1, t_{R} = 1.98 min ¹H NMR (400 MHz, DMSO) 13.06 (1H, s), 8.33 (1H, t, J = 5.3 Hz), 7.72-7.70 (1H, m), 7.60 (1H, s), 7.49-7.46 (1H, m), 7.40- 7.35 (1H, m), 7.25-7.17 (2H, m), 6.98 (1H, dd, J = 2.3, 8.9 Hz), 4.32-4.26 (2H, m), 3.22-3.19 (2H, m), 3.06-3.02 (2H, m), 2.61-2.57 (2H, m), 2.01-1.94 (2H, m) ppm. |
| Example | 14-oxa-10,19,20-triazatetracyclo[13.5.2.1^{2,6}.0^{18,21}]tricosa- | |
| 167 | 1(20),2(23),3,5,15(22),16,18(21)-heptaene | |
| | 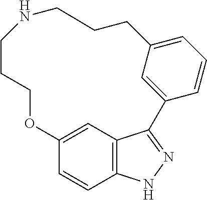 [SMILES: c1cc2cc(c1)-c1n[nH]c3ccc(cc13)OCCCNCCC2] | LCMS method F: [M + H]⁺ = 308.2, t_{R} = 1.33 min LCMS method G: [M + H]⁺ = 308.2, t_{R} = 2.37 min ¹H NMR (400 MHz, DMSO) 13.02-13.00 (1H, m), 8.09 (1H, s), 7.76-7.70 (2H, m), 7.48-7.38 (2H, m), 7.20-7.16 (1H, m), 6.99 (1H, dd, J = 1.9, 9.1 Hz), 4.39 (2H, t, J = 7.0 Hz), 2.86-2.81 (2H, m), 2.74-2.65 (4H, m), 1.95-1.89 (4H, m) ppm. |
| Example | (11S,12S)-11,12-dimethyl-7,14-dioxa-10,19,20-triazatetracyclo | |
| 168 | [13.5.2.1^{2,6}.0^{18,21}]tricosa-1(20),2(23),3,5,15(22),16,18(21)-heptaen-9-one | |
| | 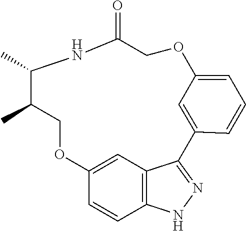 [SMILES: C[C@@H]1COc2ccc3[nH]nc(c3c2)-c2cccc(c2)OCC(=O)N[C@H]1C] | LCMS method E: [M + H]⁺ = 352.1, t_{R} = 2.72 min LCMS method D: [M + H]⁺ = 352.1, t_{R} = 3.04 min ¹H NMR (400 MHz, DMSO) 13.08 (s, 1H), 8.33 (d, J = 7.5 Hz, 1H), 7.48 (d, J = 8.9 Hz, 1H), 7.45 (d, J = 7.6 Hz, 1H), 7.39 (d, J = 7.9 Hz, 1H), 7.36 (s, 1H), 7.31 (s, 1H), 7.06 (dd, J = 8.9, 1.5 Hz, 1H), 6.93 (dd, J = 8.0, 1.3 Hz, 1H), 4.69 (q, J = 16.2 Hz, 2H), 4.32 (dd, J = 12.0, 3.6 Hz, 1H), 3.88 (dd, J = 11.9, 7.6 Hz, 1H), 3.76-3.61 (m, 1H), 2.21-2.05 (m, 1H), 1.18 (d, J = 6.7 Hz, 3H), 0.97 (d, J = 6.8 Hz, 3H) ppm. |
| Example | (11S,12S)-11,12-dimethyl-7,14-dioxa-10,19,20-triazatetracyclo | |
| 169 | [13.5.2.1^{2,6}.0^{18,21}]tricosa-1(20),2(23),3,5,15(22),16,18(21)-heptaene | |
| | 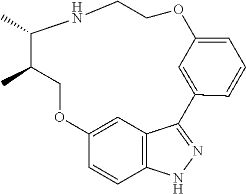 [SMILES: C[C@@H]1COc2ccc3[nH]nc(c3c2)-c2cccc(c2)OCCN[C@H]1C] | LCMS method E: [M + H]⁺ = 338.2, t_{R} = 1.84 min LCMS method D: [M + H]⁺ = 338.1, t_{R} = 3.78 min ¹H NMR (400 MHz, DMSO) 13.00 (s, 1H), 8.16 (s, 1H), 8.07 (s, 1H), 7.54 (d, J = 7.7 Hz, 1H), 7.44 (d, J = 9.0 Hz, 1H), 7.32 (t, J = 7.9 Hz, 1H), 7.00 (dd, J = 9.0, 2.1 Hz, 1H), 6.83 (dd, J = 8.1, 1.6 Hz, 1H), 4.76 (d, J = 11.8 Hz, 1H), 4.60-4.48 (m, 1H), 4.34-4.24 (m, 1H), 3.86-3.75 (m, 1H), 3.41-3.33 (m, 1H), 2.73- 2.59 (m, 1H), 2.41-2.30 (m, 1H), 2.04-1.92 (m, 1H), 1.05 (d, J = 6.4 Hz, 3H), 0.91 (d, J = 6.6 Hz, 3H) ppm. |
| Example | 12-methoxy-7,14-dioxa-10,19,20-triazatetracyclo[13.5.2.1^{2,6}.0^{18,21}]tricosa- | |
| 170 | 1(20),2(23),3,5,15,17,21-heptaen-9-one | |
| | 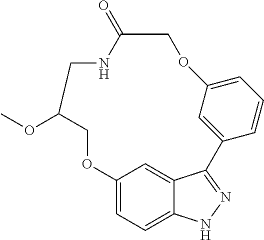 [SMILES: COC1CNC(=O)COc2cccc(c2)-c2n[nH]c3ccc(cc23)OC1] | LCMS method C: [M + H]⁺ = 354.1, t_{R} = 2.33 min LCMS method D: [M + H]⁺ = 354.1, t_{R} = 2.62 min ¹H NMR (400 MHz, DMSO) 13.15 (s, 1H), 8.60 (t, J = 6.1 Hz, 1H), 7.60 (d, J = 7.6 Hz, 1H), 7.52 (d, J = 9.0 Hz, 1H), 7.39 (t, J = 7.9 Hz, 1H), 7.29 (brt, 2H), 7.05 (dd, J = 9.0, 1.9 Hz, 1H), 6.95 (dd, J = 8.2, 1.6 Hz, 1H), 4.77 (s, 2H), 4.55 (d, J = 13.5 Hz, 1H), 4.13 (dd, J = 13.5, 8.8 Hz, 1H), 3.93-3.83 (m, 1H), 3.76-3.67 (m, 1H), 3.35 (s, 3H), 2.76 (td, J = 13.4, 5.4 Hz, 1H) ppm. |
| Example | 12-methoxy-7,14-dioxa-10,19,20-triazatetracyclo[13.5.2.1^{2,6}.0^{18,21}]tricosa- | |
| 171 | 1(20),2(23),3,5,15,17,21-heptaene | |
| | 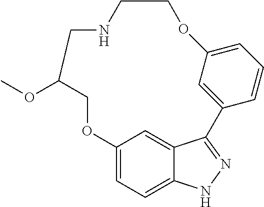 [SMILES: COC1CNCCOc2cccc(c2)-c2n[nH]c3ccc(cc23)OC1] | LCMS method E: [M + H]⁺ = 340.1, t_{R} = 1.67 min LCMS method D: [M + H]⁺ = 340.1, t_{R} = 3.32 min ¹H NMR (400 MHz, DMSO) 13.05 (s, 1H), 8.07 (s, 2H), 7.56 (d, J = 7.7 Hz, 1H), 7.47 (d, J = 9.0 Hz, 1H), 7.33 (t, J = 7.9 Hz, 1H), 7.02 (dd, J = 9.0, 2.1 Hz, 1H), 6.86 (dt, J = 8.0, 4.0 Hz, 1H), 4.66 (d, J = 13.2 Hz, 1H), 4.40 (ddd, J = 11.9, 7.5, 4.1 Hz, 1H), 4.32 (dt, J = 12.7, 4.6 Hz, 1H), 4.10 (dd, J = 13.3, 7.3 Hz, 1H), 3.82 (ddd, J = 10.2, 7.2, 2.9 Hz, 1H), 3.30 (s, 3H), 3.23- 3.13 (m, 1H), 2.96-2.87 (m, 1H), 2.87-2.77 (m, 1H), 2.40 (dd, J = 19.8, 10.8 Hz, 1H) ppm. |
| Example | (13R)-13-methyl-4-(morpholin-4-yl)-7,14-dioxa-10,19,20-triazatetracyclo | |
| 172 | [13.5.2.1^{2,6}.0^{18,21}]tricosa-1(20),2(23),3,5,15,17,21-heptaen-9-one | |
| | 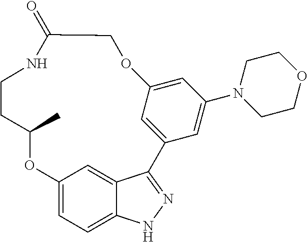 [SMILES: C[C@@H]1CCNC(=O)COc2cc(cc(N3CCOCC3)c2)-c2n[nH]c3ccc(cc23)O1] | LCMS method C: [M + H]⁺ = 423.1, t_{R} = 2.51 min LCMS method D: [M + H]⁺ = 423.2, t_{R} = 2.93 min ¹H NMR (400 MHz, DMSO) 13.03 (s, 1H), 8.58-8.47 (m, 1H), 7.45 (d, J = 9.0 Hz, 1H), 7.32 (s, 1H), 7.19 (s, 1H), 6.95 (dd, J = 8.9, 1.5 Hz, 1H), 6.83 (s, 1H), 6.50 (s, 1H), 4.68 (s, 2H), 4.55 (dd, J = 13.4, 6.6 Hz, 1H), 3.79-3.67 (m, 5H), 3.19-3.12 (m, 4H), 2.97-2.86 (m, 1H), 2.23 (t, J = 13.1 Hz, 1H), 1.49 (t, J = 14.1 Hz, 1H), 1.41 (d, J = 5.9 Hz, 3H) ppm. |
| Example | (13R)-13-methyl-4-(morpholin-4-yl)-7,14-dioxa-10,19,20-triazatetracyclo | |
| 173 | [13.5.2.1^{2,6}.0^{18,21}]tricosa-1(20),2(23),3,5,15,17,21-heptaene | |
| | 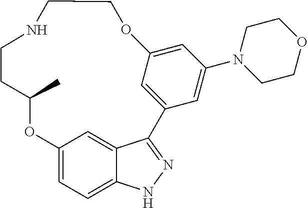 [SMILES: C[C@@H]1CCNCCOc2cc(cc(N3CCOCC3)c2)-c2n[nH]c3ccc(cc23)O1] | LCMS method C: [M + H]⁺ = 409.1, t_{R} = 1.95 min LCMS method D: [M + H]⁺ = 409.2, t_{R} = 3.44 min ¹H NMR (400 MHz, DMSO) d 12.95 (s, 1H), 8.04 (s, 1H), 7.56 (s, 1H), 7.42 (d, J = 8.9 Hz, 1H), 7.14 (d, J = 1.2 Hz, 1H), 6.94 (dd, J = 9.0, 2.0 Hz, 1H), 6.44 (t, J = 2.0 Hz, 1H), 4.69-4.60 (m, 1H), 4.38-4.26 (m, 2H), 3.78-3.68 (m, 4H), 3.18-3.10 (m, 4H), 3.09-3.03 (m, J = 10.6, 6.0 Hz, 1H), 2.87-2.78 (m, 2H), 2.63- 2.55 (m, J = 14.3, 6.8 Hz, 1H), 2.24-2.08 (m, J = 24.3, 12.7 Hz, 2H), 1.51-1.42 (m, 1H), 1.35 (d, J = 6.1 Hz, 3H) ppm. |
| Example | (13S)-13-methyl-4-(morpholin-4-yl)-7,14-dioxa-10,19,20-triazatetracyclo | |
| 174 | [13.5.2.1^{2,6}.0^{18,21}]tricosa-1(20),2(23),3,5,15,17,21-heptaen-9-one | |
| | 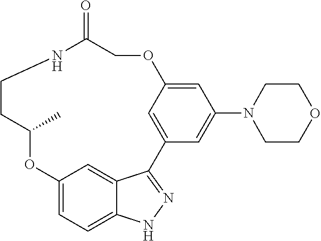 [SMILES: C[C@H]1CCNC(=O)COc2cc(cc(N3CCOCC3)c2)-c2n[nH]c3ccc(cc23)O1] | LCMS method E: [M + H]⁺ = 423.1, t_{R} = 2.39 min LCMS method D: [M + H]⁺ = 423.1, t_{R} = 2.91 min ¹H NMR (400 MHz, DMSO) 13.04 (s, 1H), 8.62-8.47 (m, 1H), 7.46 (d, J = 9.0 Hz, 1H), 7.32 (brs, 1H), 7.21-7.17 (m, 1H), 6.95 (dd, J = 8.9, 2.0 Hz, 1H), 6.82 (s, 1H), 6.50 (t, J = 2.1 Hz, 1H), 4.68 (s, 2H), 4.60-4.49 (m, 1H), 3.79-3.72 (m, 4H), 3.72-3.64 (m, 1H), 3.19-3.10 (m, 4H), 2.98-2.85 (m, 1H), 2.23 (t, J = 12.8 Hz, 1H), 1.48 (td, J = 11.7, 1.9 Hz, 1H), 1.41 (d, J = 6.0 Hz, 3H) ppm. |
| Example | (13S)-13-methyl-4-(morpholin-4-yl)-7,14-dioxa-10,19,20-triazatetracyclo | |
| 175 | [13.5.2.1^{2,6}.0^{18,21}]tricosa-1(20),2(23),3,5,15,17,21-heptaene | |
| | 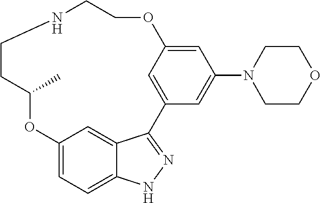 [SMILES: C[C@H]1CCNCCOc2cc(cc(N3CCOCC3)c2)-c2n[nH]c3ccc(cc23)O1] | LCMS method E: [M + H]⁺ = 409.1, t_{R} = 1.78 min LCMS method D: [M + H]⁺ = 409.2, t_{R} = 3.52 min ¹H NMR (400 MHz, DMSO) 12.95 (s, 1H), 8.04 (s, 1H), 7.56 (s, 1H), 7.43 (d, J = 8.9 Hz, 1H), 7.14 (s, 1H), 6.95 (dd, J = 8.9, 2.1 Hz, 1H), 6.44 (s, 1H), 4.70-4.57 (m, 1H), 4.37-4.23 (m, 2H), 3.78-3.64 (m, 4H), 3.18-3.00 (m, 4H), 2.81 (brt, 2H), 2.58 (dd, J = 14.2, 7.9 Hz, 1H), 2.27-2.09 (m, J = 22.9, 11.0 Hz, 2H), 1.53-1.42 (m, 1H), 1.37 (d, J = 21.9 Hz, 3H) ppm. |
| Example | 8-methyl-4-(morpholin-4-yl)-7,14-dioxa-10,19,20-triazatetracyclo | |
| 176 | [13.5.2.1^{2,6}.0^{18,21}]tricosa-1(20),2,4,6(23),15,17,21-heptaen-9-one | |
| | 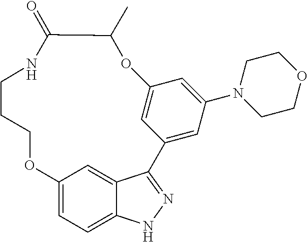 [SMILES: CC1Oc2cc(cc(N3CCOCC3)c2)-c2n[nH]c3ccc(cc23)OCCCNC1=O] | LCMS method C: [M + H]⁺ = 423.1, t_{R} = 2.41 min LCMS method D: [M + H]⁺ = 423.1, t_{R} = 2.80 min ¹H NMR (400 MHz, DMSO) 13.03 (s, 1H), 8.71-8.45 (m, J = 6.6, 4.2 Hz, 1H), 7.46 (d, J = 9.0 Hz, 1H), 7.37 (s, 1H), 7.21 (s, 1H), 6.97 (dd, J = 9.0, 2.0 Hz, 1H), 6.82 (s, 1H), 6.48 (s, 1H), 4.79 (q, J = 6.6 Hz, 1H), 4.34 (t, J = 8.1 Hz, 2H), 3.83 - 3.65 (m, 5H), 3.19-3.10 (m, 4H), 2.77 (t, J = 13.0 Hz, 1H), 2.19- 2.04 (m, 1H), 1.88-1.72 (m, 1H), 1.50 (d, J = 6.6 Hz, 3H) ppm. |
| Example | (8R) or (8S)-8-methyl-4-(morpholin-4-yl)-7,14-dioxa-10,19,20- | |
| 177 | triazatetracyclo[13.5.2.1^{2,6}.0^{18,21}]tricosa-1(20),2,4,6(23),15,17,21-heptaene | |
| | 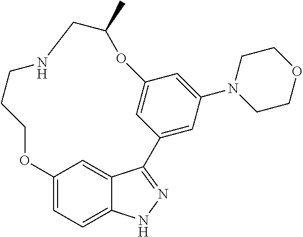 [SMILES: C[C@@H]1CNCCCOc2ccc3[nH]nc(c3c2)-c2cc(cc(N3CCOCC3)c2)O1] | Absolute stereochemistry unknown LCMS method E: [M + H]⁺ = 409.2, t_{R} = 1.76 min LCMS method D: [M + H]⁺ = 409.2, t_{R} = 3.49 min ¹H NMR (400 MHz, DMSO) 12.99 (s, 1H), 7.90 (s, 1H), 7.44 (d, J = 8.9 Hz, 1H), 7.38 (s, 1H), 7.14 (s, 1H), 6.97 (dd, J = 9.0, 1.9 Hz, 1H), 6.42 (t, J = 2.0 Hz, 1H), 4.56-4.42 (m, 2H), 4.37- 4.20 (m, J = 12.1, 8.8, 6.3 Hz, 1H), 3.79-3.66 (m, 4H), 3.23- 3.16 (m, 1H), 3.17-3.10 (m, 4H), 2.78-2.70 (m, 1H), 2.17-2.04 (m, J = 13.6, 8.0, 3.5 Hz, 2H), 1.83-1.66 (m, J = 10.6, 4.6 Hz, 1H), 1.43-1.36 (m, J = 6.2 Hz, 3H) ppm. |
| Example | (8R) or (8S)-8-methyl-4-(morpholin-4-yl)-7,14-dioxa-10,19,20- | |
| 178 | triazatetracyclo[13.5.2.1^{2,6}.0^{18,21}]tricosa-1(20),2,4,6(23),15,17,21-heptaene | |
| | 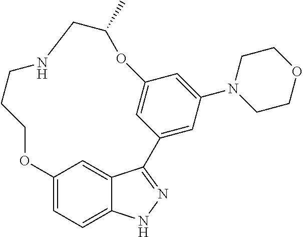 [SMILES: C[C@H]1CNCCCOc2ccc3[nH]nc(c3c2)-c2cc(cc(N3CCOCC3)c2)O1] | Absolute stereochemistry unknown LCMS method E: [M + H]⁺ = 409.2, t_{R} = 1.75 min LCMS method D: [M + H]⁺ = 409.2, t_{R} = 3.47 min ¹H NMR (400 MHz, DMSO) 12.99 (s, 1H), 7.90 (s, 1H), 7.44 (d, J = 8.9 Hz, 1H), 7.38 (s, 1H), 7.14 (s, 1H), 6.97 (dd, J = 8.9, 1.9 Hz, 1H), 6.42 (t, J = 1.8 Hz, 1H), 4.57-4.41 (m, 2H), 4.37- 4.22 (m, 1H), 3.77-3.68 (m, 4H), 3.21-3.17 (m, J = 2.5 Hz, 1H), 3.16-3.09 (m, 4H), 2.82-2.70 (m, J = 8.4, 6.5 Hz, 1H), 2.17- 2.04 (m, J = 15.5, 6.7 Hz, 2H), 1.81-1.67 (m, J = 13.7, 6.4 Hz, 1H), 1.40 (d, J = 6.2 Hz, 3H) ppm. |
| Example | (12R)-12-methyl-13-oxa-4,5,9,18,19-pentaazatetracyclo [12.5.2.1^{2,5}.0^{17,20}] docosa- | |
| 179 | 1(19),2(22),3,14(21),15,17(20)-hexaen-8-one | |
| | 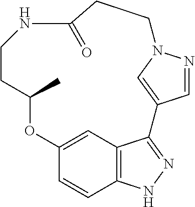 | LCMS method F: [M + H]⁺ = 326.3, t_{R} = 1.61 min LCMS method G: [M + H]⁺ = 326.3, t_{R} = 1.61 min ¹H NMR (400 MHz, DMSO) 12.77 (1H, s), 8.56-8.53 (1H, m), 7.97 (1H, s), 7.67 (1H, s), 7.41-7.38 (1H, d), 7.01 (1H, m), 6.93- 6.91 (1H, dd), 4.70-4.63 (1H, m), 4.53-4.49 (1H, m), 4.39-4.33 (1H, m), 3.90-3.84 (1H, m), 2.87-2.78 (1H, m), 2.74-2.70 (2H, m), 2.30-2.25 (1H, m), 1.46-1.38 (1H, m), 1.41 (3H, d) ppm. |
| Example | (12R)-12-methyl-13-oxa-4,5,9,18,19-pentaazatetracyclo [12.5.2.1^{2,5}.0^{17,20}]docosa- | |
| 180 | 1(19),2(22),3,14(21),15,17(20)-hexaene | |
| | 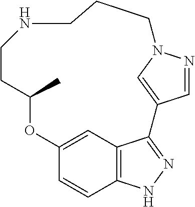 | LCMS method F: [M + H]⁺ = 312.3, t_{R} = 1.00 min LCMS method G: [M + H]⁺ = 312.3, t_{R} = 1.93 min ¹H NMR (400 MHz, MeOD) 12.77 (1H, s), 8.72 (1H, s), 7.77 (1H, m), 7.50 (1H, m), 7.40-7.38 (1H, d), 7.02-7.00 (1H, dd), 4.62-4.55 (1H, m), 4.52-4.46 (1H, m), 4.40-4.34 (1H, m), 2.85- 2.77 (3H, m), 2.70-2.64 (1H, m), 2.35-2.28 (1H, m), 2.17-2.10 (2H, m), 1.56-1.48 (1H, m), 1.43 (3H, d) ppm. |
| Example | 13-oxa-4,5,9,18,19,22-hexaazatetracyclo[12.5.2.1^{2,5}.0^{17,20}] docosa- | |
| 181 | 1(19),2(22),3,14(21),15,17(20)-hexaen-8-one | |
| | 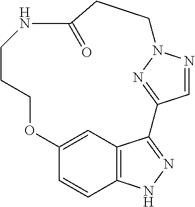 [SMILES: O=C1CCCn2ncc(n2)-c2n[nH]c3ccc(cc23)OCCCN1] | LCMS method F: [M + H]⁺ = 313.3, t_{R} = 1.48 min LCMS method G: [M + H]⁺ = 313.3, t_{R} = 1.49 min ¹H NMR (400 MHz, DMSO) 13.06 (1H, s), 8.55-8.50 (1H, m), 8.13 (1H, s), 7.84 (1H, m), 7.46-7.42 (1H, d, J = 9.0 Hz), 6.99- 6.95 (1H, dd, J = 2.5, 9.0 Hz), 4.76-4.71 (2H, m), 4.36-4.29 (2H, m), 3.29-3.18 (2H, m), 2.93-2.87 (2H, m), 2.01-1.91 (2H, m) ppm. |
| Example | 13-oxa-4,5,9,18,19,22-hexaazatetracyclo[12.5.2.1^{2,5}.0^{17,20}] docosa- | |
| 182 | 1(19),2(22),3,14(21),15,17(20)-hexaene | |
| | 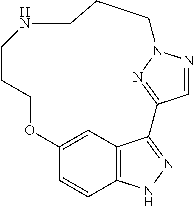 [SMILES: c1cc2[nH]nc3c2cc1OCCCNCCCCn1ncc-3n1] | LCMS method F: [M + H]⁺ = 299.3, t_{R} = 1.07 min LCMS method G: [M + H]⁺ = 299.3, t_{R} = 1.74 min ¹H NMR (400 MHz, DMSO) 13.04 (1H, s), 8.12-8.05 (2H, m), 7.49-7.41 (1H, d, J-9.1 Hz), 7.02-6.95 (1H, d, J = 9.1 Hz), 4.62- 4.54 (2H, m), 4.43-4.35 (2H, m), 3.01-2.91 (2H, m), 2.79-2.70 (2H, m), 2.22-2.12 (2H, m), 1.97-1.88 (2H, m) ppm. |
| Example | (11S,12S)-12-fluoro-11-methyl-7,14-dioxa-10,19,20-triazatetracyclo | |
| 183 | [13.5.2.1^{2,6}.0^{18,21}]tricosa-1(20),2,4,6(23),15,17,21-heptaen-9-one | |
| | 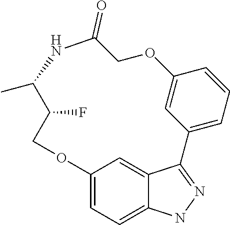 [SMILES: C[C@@H]1NC(=O)COc2cccc(c2)-c2n[nH]c3ccc(cc23)OC[C@H]1F] | LCMS method E: [M + H]⁺ = 356.1, t_{R} = 2.63 min LCMS method D: [M + H]⁺ = 356.1, t_{R} = 2.97 min ¹H NMR (400 MHz, DMSO) 13.12 (s, 1H), 8.43 (d, J = 8.2 Hz, 1H), 7.51 (d, J = 9.0 Hz, 1H), 7.47-7.37 (m, 2H), 7.29 (dd, J = 2.7, 1.4 Hz, 1H), 7.24 (d, J = 1.9 Hz, 1H), 7.07 (dd, J = 9.0, 2.2 Hz, 1H), 6.96 (ddd, J = 8.0, 2.5, 1.2 Hz, 1H), 4.86-4.49 (m, 4H), 4.45-4.30 (m, 1H), 4.02 (dd, J = 13.8, 7.6 Hz, 1H), 1.25 (dd, J = 6.8, 4.2 Hz, 3H) ppm. |
| Example | (11S,12S)-12-fluoro-11-methyl-7,14-dioxa-10,19,20- | |
| 184 | triazatetracyclo[13.5.2.1^{2,6}.0^{18,21}]tricosa-1(20),2,4,6(23),15,17,21-heptaene | |
| | 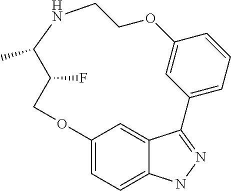 [SMILES: C[C@@H]1NCCOc2cccc(c2)-c2n[nH]c3ccc(cc23)OC[C@H]1F] | LCMS method E: [M + H]⁺ = 342.1, t_{R} = 1.90 min LCMS method D: [M + H]⁺ = 342.1, t_{R} = 3.75 min ¹H NMR (400 MHz, DMSO) 13.05 (s, 1H), 8.14-.11 (m, 2H), 7.57 (d, J = 7.7 Hz, 1H), 7.49 (d, J = 9.0 Hz, 1H), 7.32 (t, J = 7.9 Hz, 1H), 7.05 (dd, J = 9.0, 2.2 Hz, 1H), 6.87-6.82 (m, 1H), 4.94-4.62 (m, 2H), 4.57-4.45 (m, 1H), 4.41-4.23 (m, 2H), 3.46- 3.34 (m, 1H), 2.85-2.73 (m, 1H), 2.73-2.62 (m, 1H), 2.17 (td, J = 10.7, 5.0 Hz, 1H), 1.16 (dd, J = 6.3, 3.3 Hz, 3H) ppm. |
| Example | 4-(trifluoromethyl)-7,14-dioxa-10,19,20-triazatetracyclo [13.5.2.1^{2,6}.0^{18,21}]tricosa- | |
| 185 | 1(20),2,4,6(23),15,17,21-heptaen-9-one | |
| | 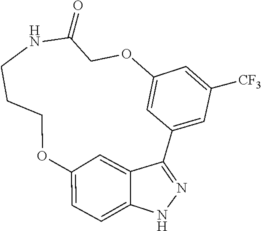 [SMILES: O=C1COc2cc(cc(C(F)(F)F)c2)-c2n[nH]c3ccc(cc23)OCCCN1] | LCMS method E: [M + H]⁺ = 392.1, t_{R} = 2.90 min ¹H NMR (400 MHz, DMSO) 13.35 (s, 1H), 8.57 (t, J = 5.9 Hz, 1H), 7.87 (s, 1H), 7.59 (s, 1H), 7.52 (d, J = 9.0 Hz, 1H), 7.33 (d, J = 1.8 Hz, 1H), 7.24 (s, 1H), 7.02 (dd, J = 9.0, 2.1 Hz, 1H), 4.89 (s, 2H), 4.36 (t, J = 8.2 Hz,, 2H), 3.31-3.17 (m, 2H), 2.11- 1.90 (m, 2H) ppm. |
| Example | 4-(trifluoromethyl)-7,14-dioxa-10,19,20-triazatetracyclo [13.5.2.1^{2,6}.0^{18,21}]tricosa- | |
| 186 | 1(20),2,4,6(23),15,17,21-heptaene | |
| | 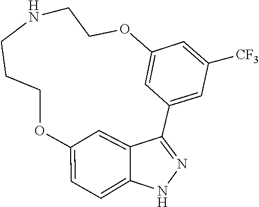 [SMILES: FC(F)(F)c1cc2cc(c1)-c1n[nH]c3ccc(cc13)OCCCNCCO2] | LCMS method E: [M + H]⁺ = 378.1, t_{R} = 2.06 min ¹H NMR (400 MHz, DMSO) 13.26 (s, 1H), 8.34 (s, 1H), 8.12 (d, J = 1.7 Hz, 1H), 7.83 (s, 1H), 7.49 (d, J = 9.0 Hz, 1H), 7.15 (s, 1H), 7.01 (dd, J = 9.0, 2.1 Hz, 1H), 4.45 (t, J = 5.4 Hz, 2H), 4.40 (t, J = 7.27 Hz, 2H) 3.05-2.92 (m, 2H), 2.71-2.60 (m, 2H), 2.03-1.79 (m, 2H) ppm. |
| Example | 16-methyl-7,14-dioxa-4,10,19,20-tetraazatetracyclo[13.5.2.1^{2,6}.0^{18,21}]tricosa- | |
| 187 | 1(20),2(23),3,5,15(22),16,18(21)-heptaen-9-one | |
| | 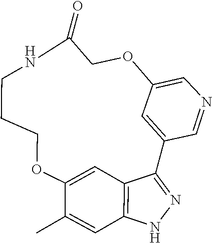 [SMILES: Cc1cc2[nH]nc3c2cc1OCCCNC(=O)COc1cncc-3c1] | LCMS method E: [M + H]⁺ = 339.1, t_{R} = 1.87 min ¹H NMR (300 MHz, DMSO) 13.15 (s, 1H), 8.76 (s, 1H), 8.59 (s, 1H), 8.26 (s, 1H), 7.68 (s, 1H), 7.40 (s, 1H), 7.26 (s, 1H), 4.85 (s, 2H), 4.40 (t, J = 7.4 Hz, 2H), 3.24 (s, 2H), 2.28 (s, 3H), 2.00 (s, 2H) ppm. |
| Example | 16-methyl-7,14-dioxa-4,10,19,20-tetraazatetracyclo[13.5.2.1^{2,6}.0^{18,21}]tricosa- | |
| 188 | 1(20),2(23),3,5,15(22),16,18(21)-heptaene | |
| | 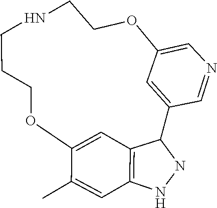 [SMILES: Cc1cc2c3cc1OCCCNCCOc1cncc(c1)C3NN2] | LCMS method E: [M + H]⁺ = 325.0, t_{R} = 2.42 min LCMS method D: [M + H]⁺ = 325.0, t_{R} = 3.64 min ¹H NMR (300 MHz, DMSO) 13.06 (s, 1H), 8.72 (s, 1H), 8.49 (s, 1H), 8.18 (s, 1H), 8.04 (s, 1H), 7.36 (s, 1H), 4.42 (t, J = 4.9 Hz, 4H), 2.97 (brs, 2H), 2.73 (s, 1H), 2.64 (brs, 2H), 2.28 (s, 3H), 1.94 (brs, 2H) ppm. |
| Example | 10-methyl-7,14-dioxa-5,10,19,20-tetraazatetracyclo[13.5.2.1^{2,6}.0^{18,21}]tricosa- | |
| 189 | 1(20),2(23),3,5,15(22),16,18(21)-heptaen-9-one | |
| | 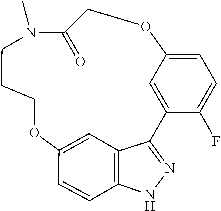 [SMILES: CN1CCCOc2ccc3[nH]nc(c3c2)-c2cc(ccc2F)OCC1=O] | LCMS method E: [M + H]⁺ = 356.0, t_{R} = 2.42 min LCMS method D: [M + H]⁺ = 356.0, t_{R} = 3.09 min ¹H NMR (300 MHz, DMSO) 13.16 (s, 1H), 7.49 (d, J = 8.9 Hz, 1H), 7.32 (dd, J = 5.1, 3.0 Hz, 1H), 7.28-7.15 (m, 2H), 7.04- 6.88 (m, 2H), 5.15 (d, J = 16.6 Hz, 1H), 4.97 (d, J = 16.1 Hz, 1H), 4.48-4.30 (m, 1H), 4.30-4.13 (m, 1H), 4.10-3.93 (m, 1H), 3.00 (s, 3H), 2.74 (t, J = 12.0 Hz, 1H), 2.39 (brt, 1H), 1.71 (brt, J = 14.2 Hz, 2H) ppm. |
| Example | 10-methyl-7-oxa-4,10,14,19,20-pentaazatetracyclo[13.5.2.1^{2,6}.0^{18,21}]tricosa- | |
| 190 | 1(20),2,4,6(23),15,17,21-heptaen-9-one | |
| | 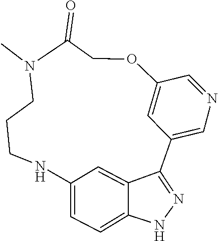 [SMILES: CN1CCCNc2ccc3[nH]nc(c3c2)-c2cncc(c2)OCC1=O] | LCMS method E: [M + H]⁺ = 338.1, t_{R} = 1.34 min LCMS method D: [M + H]⁺ = 338.1, t_{R} = 2.50 min ¹H NMR (300 MHz, DMSO) 12.92 (s, 1H), 8.64 (s, 1H), 8.21 (s, 1H), 7.75 (s, 1H), 7.32 (d, J = 8.8 Hz, 1H), 6.80 (d, J = 9.2 Hz, 1H), 6.77 (s, 1H), 5.84 (t, 1H), 5.27 (d, J = 16.5 Hz, 1H), 5.06 (d, J = 16.6 Hz, 1H), 3.99 (d, J = 13.4 Hz, 1H), 3.24-3.14 (m, 2H), 3.12 (s, 3H), 2.70 (t, J = 12.1 Hz, 1H), 2.39-2.24 (m, 1H), 1.42 (dd, J = 16.3, 9.6 Hz, 2H) ppm. |
| Example | 10-methyl-9-oxo-7,14-dioxa-10,19,20-triazatetracyclo [13.5.2.1^{2,6}.0^{18,21}]tricosa- | |
| 191 | 1(20),2,4,6(23),15,17,21-heptaene-4-carbonitrile | |
| | 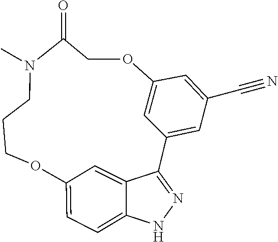 [SMILES: CN1CCCOc2ccc3[nH]nc(c3c2)-c2cc(C#N)cc(c2)OCC1=O] | LCMS method E: [M + H]⁺ = 363.1, t_{R} = 1.68 min ¹H NMR (300 MHz, DMSO) 7.76 (d, J = 10.6 Hz, 2H), 7.50 (d, J = 8.9 Hz, 1H), 7.38 (s, 1H), 7.25 (s, 1H), 7.00 (d, J = 8.6 Hz, 1H), 5.33 (d, J = 16.5 Hz, 1H), 5.10 (d, J = 16.6 Hz, 1H), 4.45-4.20 (m, 2H), 4.02 (d, J = 13.1 Hz, 2H), 3.07 (s, 3H), 1.72 (brs, 2H) ppm. |
| Example | 7,13-dioxa-10,18,19-triazatetracyclo[12.5.2.1^{2,6}.0^{17,20}]docosa- | |
| 192 | 1(19),2(22),3,5,14(21),15,17(20)-heptaen-9-one | |
| | 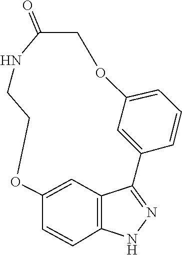 [SMILES: O=C1COc2cccc(c2)-c2n[nH]c3ccc(cc23)OCCN1] | LCMS method F: [M + H]⁺ = 310.1, t_{R} = 1.74 min LCMS method G: [M + H]⁺ = 310.2, t_{R} = 1.71 min ¹H NMR (400 MHz, MeOD) 7.52-7.40 (4H, m), 7.33 (1H, m), 7.10-7.05 (2H, m), 4.70-4.69 (2H, s), 3.67-3.62 (2H, m), 1.33- 1.30 (3H, m) ppm. |
| Example | 7,14-dioxa-4,10,19,20,23-pentaazatetracyclo[13.5.2.1^{2,6}.0^{18,21}]tricosa- | |
| 193 | 1(20),2(23),3,5,15(22),16,18(21)-heptaen-9-one | |
| | 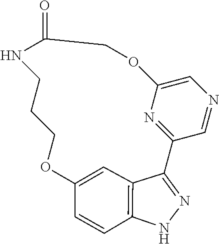 [SMILES: O=C1COc2cncc(n2)-c2n[nH]c3ccc(cc23)OCCCN1] | LCMS method F: [M + H]⁺ = 326.1, t_{R} = 1.70 min ¹H NMR (400 MHz, DMSO) 13.47 (1H, s), 8.96 (1H, s), 8.50 (1H, t, J = 6.0 Hz), 8.29 (1H, s), 7.67 (1H, d, J = 1.9 Hz), 7.54- 7.50 (1H, m), 7.01 (1H, dd, J = 2.2, 9.0 Hz), 4.97-4.95 (2H, m), 4.32 (2H, t, J = 7.8 Hz), 3.24-3.17 (2H, m), 1.97-1.94 (2H, m) ppm. |
| Example | 10-methyl-7,14-dioxa-3,10,19,20-tetraazatetracyclo[13.5.2.1^{2,6}.0^{18,21}]tricosa- | |
| 194 | 1(20),2(23),3,5,15(22),16,18(21)-heptaen-9-one | |
| | 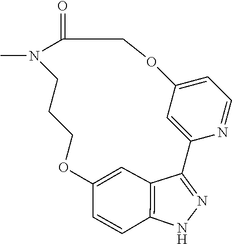 [SMILES: CN1CCCOc2ccc3[nH]nc(c3c2)-c2cc(ccn2)OCC1=O] | LCMS method C: [M + H]⁺ = 339.1, t_{R} = 1.61 min LCMS method D: [M + H]⁺ = 339.1, t_{R} = 3.10 min ¹H NMR (300 MHz, DMSO) 13.88 (s, 1H), 8.53 (d, J = 6.2 Hz, 1H), 7.80 (s, 1H), 7.61 (d, J = 9.0 Hz, 1H), 7.29 (s, J = 6.2 Hz, 1H), 7.22 (s, 1H), 7.08 (d, J = 8.8 Hz, 1H), 5.60-5.30 (m, 2H), 4.50-4.25 (m, 2H), 4.00 (d, J = 14.0 Hz, 1H), 3.08 (s, 3H), 2.93- 2.66 (m, 2H), 1.82-1.60 (m, J = 15.8 Hz, 1H) ppm. |
| Example | 4-fluoro-7,14-dioxa-10,19,20-triazatetracyclo[13.5.2.1^{2,6}.0^{18,21}]tricosa- | |
| 195 | 1(20),2(23),3,5,15(22),16,18(21)-heptaen-9-one | |
| | 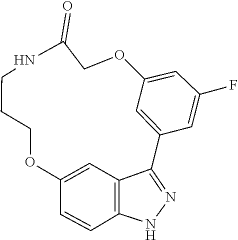 [SMILES: O=C1COc2cc(F)cc(c2)-c2n[nH]c3ccc(cc23)COCCCN1] | LCMS method F: [M + H]⁺ = 342.1, t_{R} = 1.99 min LCMS method G: [M + H]⁺ = 342.2, t_{R} = 1.77 min ¹H NMR (400 MHz, MeOD) 7.48 (1H, d), 7.43 (1H, m), 7,38 (1H, m), 7.28 (1H, m), 7.06 (1H, dd, J = 2.4, 9.0 Hz), 6.79-6.75 (1H, m), 4.82 (2H, s), 4.39 (2H, m), 3.41 (2H, m), 2.19-2.12 (2H, m) ppm. |
| Example | 7,14-dioxa-5,10,19,20,23-pentaazatetracyclo[13.5.2.1^{2,6}.0^{18,21}]tricosa- | |
| 196 | 1(20),2(23),3,5,15(22),16,18(21)-heptaen-9-one | |
| | 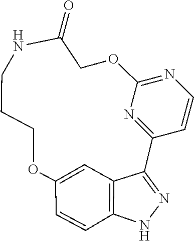 [SMILES: O=C1COc2nccc(n2)-c2n[nH]c3ccc(cc23)OCCCN1] | LCMS method F: [M + H]⁺ = 326.1, t_{R} = 1.65 min ¹H NMR (400 MHz, DMSO) 13.68-13.65 (1H, m), 8.61 (1H, d, J = 5.1 Hz), 8.46 (1H, t, J-5.9 Hz), 7.79 (1H, d, J = 5.1 Hz), 7.69 (1H, d, J = 2.1 Hz), 7.55-7.52 (1H, m), 7.02 (1H, dd, J = 2.4, 9.0 Hz), 4.96-4.94 (2H, m), 4.32 (2H, t, J = 8.0 Hz), 3.23-3.16 (2H, m), 1.91 (2H, s) ppm. |
| Example | 8,8,10-trimethyl-7,14-dioxa-4,10,19,20-tetraazatetracyclo | |
| 197 | [13.5.2.1^{2,6}.0^{18,21}]tricosa-1(20),2(23),3,5,15(22),16,18(21)-heptaen-9-one | |
| | 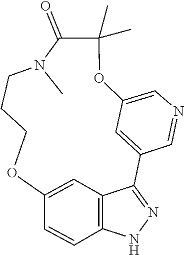 [SMILES: CN1CCCOc2ccc3[nH]nc(c3c2)-c2cncc(c2)OC(C)(C)C1=O] | LCMS method E: [M + H]⁺ = 367.2, t_{R} = 2.46 min LCMS method D: [M + H]⁺ = 367.2, t_{R} = 4.18 min ¹H NMR (300 MHz, DMSO) 13.32 (s, 1H), 8.74 (d, J = 1.4 Hz, 1H), 8.29 (d, J = 2.7 Hz, 1H), 7.63 (s, 1H), 7.53 (d, J = 9.0 Hz, 1H), 7.10 (s, 1H), 7.01 (dd, J = 9.0, 2.1 Hz, 1H), 4.53-4.28 (m, 2H), 4.05-3.90 (m, 1H), 3.40 (s, 3H), 2.86-2.68 (m, 1H), 2.28- 2.11 (m, 1H), 2.03-1.88 (m, 1H), 1.68 (s, 3H), 1.41 (s, 3H) ppm. |
| Example | 11-methyl-7,15-dioxa-4,11,20,21-tetraazatetracyclo [14.5.2.1^{2,6}.0^{19,22}] tetracosa- | |
| 198 | 1(21),2,4,6(24),16,18,22-heptaen-10-one | |
| | 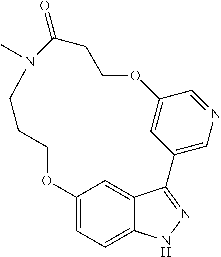 [SMILES: CN1CCCOc2ccc3[nH]nc(c3c2)-c2cncc(c2)OCCC1=O] | LCMS method E: [M + H]⁺ = 353.2, t_{R} = 1.96 min LCMS method D: [M + H]⁺ = 353.1, t_{R} = 4.09 min ¹H NMR (300 MHz, DMSO, 100° C.) 12.96 (s, 1H), 8.62 (brs, 1H), 8.26 (d, J = 2.8 Hz, 1H), 7.76 (brs, 1H), 7.51 (d, J = 8.9 Hz, 1H), 7.28 (d, J = 2.0 Hz, 1H), 7.03 (dd, J = 9.0, 2.3 Hz, 1H), 4.66-4.52 (m, 3H), 4.21-4.17 (m, 3H), 3.46 (dd, J = 13.0, 6.1 Hz, 2H), 2.83 (m, 3H), 2.10 (brs, 2H) ppm. |
| Example | 7,13-dioxa-4,10,18,19-tetraazatetracyclo[12.5.2.1^{2,6}.0^{17,20}] docosa- | |
| 199 | 1(19),2(22),3,5,14(21),15,17(20)-heptaen-9-one | |
| | 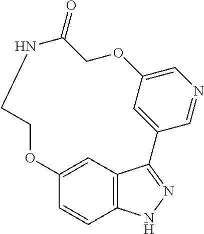 [SMILES: O=C1COc2cncc(c2)-c2n[nH]c3ccc(cc23)OCCN1] | LCMS method F: [M + H]⁺ = 311.2, t_{R} = 1.22 min LCMS method G: [M + H]⁺ = 311.2, t_{R} = 1.29 min ¹H NMR (400 MHz, MeOD) 8.59 (1H, d, J = 1.5 Hz), 8.39 (1H, d, J-2.8 Hz), 7.77 (1H, dd, J = 1.5, 2.8 Hz), 7.49-7.46 (1H, m), 7.40 (1H, d, J = 2.5 Hz), 7.11 (1H, dd, J-2.4, 9.0 Hz), 4.80 (2H, s), 3.68-3.66 (2H, m), 1.34-1.30 (2H, m) ppm. |
| Example | 3-fluoro-10-methyl-7,14-dioxa-4,10,19,20-tetraazatetracyclo | |
| 200 | [13.5.2.1^{2,6}.0^{18,21}]tricosa-1(20),2,4,6(23),15(22),16,18(21)-heptaen-9-one | |
| | 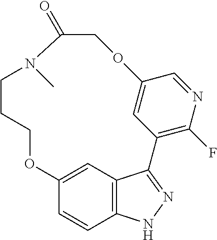 [SMILES: CN1CCCOc2ccc3[nH]nc(c3c2)-c2cc(cnc2F)OCC1=O] | LCMS method E: [M + H]⁺ = 357.1, t_{R} = 2.21 min LCMS method D: [M + H]⁺ = 357.1, t_{R} = 2.98 min ¹H NMR (300 MHz, DMSO) 13.04 (s, IH), 8.02-7.98 (m, IH), 7.90-7.88 (m, 1H), 7.51 (d, J = 9.0 Hz, 1H), 7.20 (d, J = 1.4 Hz, 1H), 7.01 (dd, J = 9.0, 2.2 Hz, 1H), 5.29-5.08 (m, 2H), 4.37- 4.24 (m, 2H), 3.99-3.95 (m, 1H), 3.01 (s, 3H), 2.78-2.73 (m, 1H), 2.50-2.35 (m, 1H), 1.74 (m, 1H) ppm. |
| Example | 8,8-dimethyl-7,14-dioxa-4,10,19,20-tetraazatetracyclo [13.5.2.1^{2,6}.0^{18,21}]tricosa- | |
| 201 | 1(20),2(23),3,5,15,17,21-heptaen-9-one | |
| | 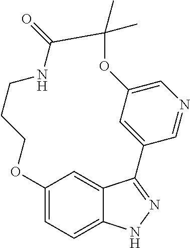 [SMILES: CC1(C)Oc2cncc(c2)-c2n[nH]c3ccc(cc23)OCCCNC1=O] | LCMS method E: [M + H]⁺ = 353.2, t_{R} = 1.88 min LCMS method D: [M + H]⁺ = 353.2, t_{R} = 3.13 min ¹H NMR (400 MHz, DMSO) 13.31 (s, 1H), 8.80 (s, 1H), 8.37 (t, J = 5.4 Hz, 1H), 8.21 (d, J = 2.5 Hz, 1H), 7.76 (s, 1H), 7.53 (d, J = 9.0 Hz, 1H), 7.22 (d, J = 1.6 Hz, 1H), 7.01 (dd, J = 9.0, 1.9 Hz, 1H), 4.32 (t, J = 8.5 Hz, 2H), 3.22 (q, J = 6.2 Hz, 2H), 2.05 (m, 2H), 1.61 (s, 6H) ppm. |
| Example | 12,12-difluoro-7,14-dioxa-4,10,19,20-tetraazatetracyclo [13.5.2.1^{2,6}.0^{18,21}]tricosa- | |
| 202 | 1(20),2,4,6(23),15,17,21-heptaen-9-one | |
| | 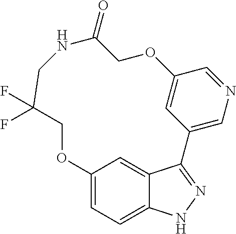 [SMILES: O=C1COc2cncc(c2)-c2n[nH]c3ccc(cc23)OCC(F)(F)CN1] | LCMS method C: [M + H]⁺ = 361.2, t_{R} = 1.99 min LCMS method D: [M + H]⁺ = 361.1, t_{R} = 2.61 min ¹H NMR (400 MHz, DMSO) 13.09 (s, 1H), 8.55-8.52 (m, 1H), 7.46-7.43 (m, 1H), 7.33 (m, 1H), 7.12 (s, 1H), 6.97-6.94 (m, 1H), 6.74 (s, 1H), 4.70 (s, 2H), 4.35-4.29 (m, 2H), 3.21 (brs, 2H) ppm. |
| Example | 4-bromo-7,14-dioxa-10,19,20-triazatetracyclo[13.5.2.1^{2,6}.0^{18,21}]tricosa- | |
| 203 | 1(20),2(23),3,5,15,17,21-heptaen-9-one | |
| | 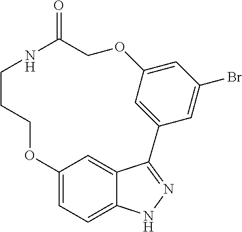 [SMILES: O=C1COc2cc(Br)cc(c2)-c2n[nH]c3ccc(cc23)OCCCN1] | LCMS method E: [M + H]⁺ = 402.0-404.0, t_{R} = 2.65 min LCMS method D: [M + H]⁺ = 402.0-404.0, t_{R} = 3.49 min ¹H NMR (300 MHz, DMSO) 13.30 (s, 1H), 8.56 (t, J = 5.8 Hz, 1H), 7.73 (s, 1H), 7.50 (d, J = 9.0 Hz, 1H), 7.32 (s, 1H), 7.31 (s, 1H), 7.15 (s, 1H), 7.00 (d, J = 9.1 Hz, 1H), 4.81 (s, 2H), 4.35 (t, J = 8.0 Hz, 2H), 3.29-3.19 (m, 2H), 2.06-1.89 (m, J = 5.3 Hz, 2H) ppm. |
| Example | 4-(piperidin-1-yl)-7,14-dioxa-10,19,20-triazatetracyclo [13.5.2.1^{2,6}.0^{18,21}]tricosa- | |
| 204 | 1(20),2(23),3,5,15,17,21-heptaen-9-one | |
| | 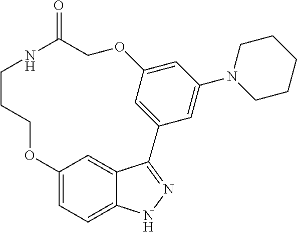 [SMILES: O=C1COc2cc(cc(N3CCCCC3)c2)-c2n[nH]c3ccc(cc23)OCCCN1] | LCMS method E: [M + H]⁺ = 407.2, t_{R} = 1.80 min LCMS method O: [M + H]⁺ = 407.2, t_{R} = 4.30 min ¹H NMR (300 MHz, DMSO) 13.02 (s, 1H), 8.54 (t, J = 5.8 Hz, 1H), 7.46 (d, J = 9.1 Hz, 1H), 7.35 (s, 1H), 7.19 (s, 1H), 6.97 (d, J = 8.8 Hz, 1H), 6.77 (s, 1H), 6.46 (s, 1H), 4.67 (s, 2H), 4.40- 4.26 (m, 2H), 3.27-3.15 (m, 6H), 2.05-1.90 (m, 2H), 1.71-1.51 (m, 6H) ppm. |
| Example | 4-(pyrrolidin-1-yl)-7,14-dioxa-10,19,20-triazatetracyclo [13.5.2.1^{2,6}.0^{18,21}]tricosa- | |
| 205 | 1(20),2(23),3,5,15,17,21-heptaen-9-one | |
| | 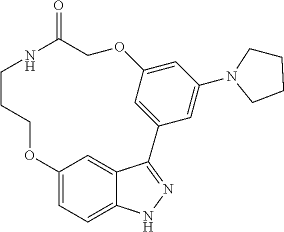 [SMILES: O=C1COc2cc(cc(N3CCCC3)c2)-c2n[nH]c3ccc(cc23)OCCCN1] | LCMS method E: [M + H]⁺ = 393.2, t_{R} = 2.62 min LCMS method D: [M + H]⁺ = 393.2, t_{R} = 3.12 min ¹H NMR (400 MHz, DMSO) 12.98 (s, 1H), 8.53 (t, J = 5.9 Hz, 1H), 7.46 (d, J = 9.0 Hz, 1H), 7.36 (s, 1H), 6.96 (dd, J = 9.0, 2.0 Hz, 1H), 6.85 (s, 1H), 6.66 (s, 1H), 6.11 (s, 1H), 4.65 (s, 2H), 4.41-4.28 (m, 2H), 3.27 (dd, J = 10.0, 4.7 Hz, 4H), 3.22 (t, J = 5.3 Hz, 2H), 2.02-1.93 (m, 6H) ppm. |
| Example | 4-(3,3-difluoropyrrolidin-1-yl)-7,14-dioxa-10,19,20-triazatetracyclo | |
| 206 | [13.5.2.1^{2,6}.0^{18,21}]tricosa-1(20),2(23),3,5,15,17,21-heptaen-9-one | |
| | 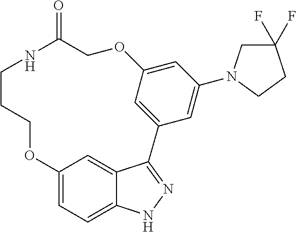 [SMILES: O=C1COc2cc(cc(N3CCC(F)(F)C3)c2)-c2n[nH]c3ccc(cc23)OCCCN1] | LCMS method E: [M + H]⁺ = 429.2, t_{R} = 2.79 min LCMS method D: [M + H]⁺ = 429.1, t_{R} = 3.09 min ¹H NMR (400 MHz, DMSO) 13.06 (s, 1H), 8.57 (t, J = 6.0 Hz, 1H), 7.47 (d, J = 9.0 Hz, 1H), 7.36 (s, 1H), 6.97 (dd, J = 9.0, 2.1 Hz, 1H), 6.88 (d, J = 1.1 Hz, 1H), 6.77 (s, 1H), 6.21 (t, J = 2.1 Hz, 1H), 4.69 (s, 2H), 4.34 (dd, J (H-F) = 13.6, 5.4 Hz, 2H), 3.74 (t, J (H-F) = 13.4 Hz, 2H), 3.53 (t, J = 7.1 Hz, 2H), 3.24 (dt, J = 10.5, 4.1 Hz, 2H), 2.63-2.52 (m, 2H), 2.02-1.93 (m, 2H) ppm. |
| Example | 4-(morpholin-4-yl)-7,14-dioxa-10,19,20-triazatetracyclo [13.5.2.1^{2,6}.0^{18,21}]tricosa- | |
| 207 | 1(20),2(23),3,5,15,17,21-heptaen-9-one | |
| | 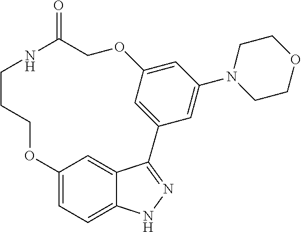 [SMILES: O=C1COc2cc(cc(N3CCOCC3)c2)-c2n[nH]c3ccc(cc23)OCCCN1] | LCMS method E: [M + H]⁺ = 409.1, t_{R} = 2.17 min LCMS method D: [M + H]⁺ = 409.2, t_{R} = 2.59 min ¹H NMR (400 MHz, DMSO) 13.06 (s, 1H), 8.55 (t, J = 5.9 Hz, 1H), 7.47 (d, J = 9.0 Hz, 1H), 7.35 (s, 1H), 7.21 (s, 1H), 6.97 (dd, J = 9.0, 2.0 Hz, 1H), 6.85 (s, 1H), 6.51 (s, 1H), 4.70 (s, 2H), 4.34 (t, J = 7.7 Hz, 2H), 3.76 (t, J = 5.3 Hz, 4H), 3.26-3.20 (m, 2H), 3.16 (t, J = 4.8 Hz, 4H), 2.05-1.90 (m, 2H) ppm. |
| Example | 4-(3,6-dihydro-2H-pyran-4-yl)-7,14-dioxa-10,19,20-triazatetracyclo | |
| 208 | [13.5.2.1^{2,6}.0^{18,21}]tricosa-1(20),2(23),3,5,15,17,21-heptaen-9-one | |
| | 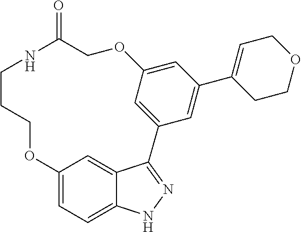 [SMILES: O=C1COc2cc(C3=CCOCC3)cc(c2)-c2n[nH]c3ccc(cc23)OCCCN1] | LCMS method E: [M + H]⁺ = 406.2, t_{R} = 2.45 min LCMS method D: [M + H]⁺ = 406.1, t_{R} = 2.84 min ¹H NMR (300 MHz, DMSO) 13.13 (s, 1H), 8.56 (t, J = 5.8 Hz, 1H), 7.69 (s, 1H), 7.48 (d, J = 9.0 Hz, 1H), 7.35 (s, 1H), 7.25 (s, 1H), 6.99 (s, 1H), 6.97 (s, 1H), 6.32 (s, 1H), 4.76 (s, 2H), 4.39-4.30 (t, J = 7.86 Hz, 2H), 4.28-4.20 (m, 2H), 3.85 (t, J = 5.3 Hz, 2H), 3.27-3.19 (m, 2H), 2.05-1.91 (m, 2H) ppm. |
| Example | 4-[4-(cyclopropylmethyl)piperazin-1-yl]-7,14-dioxa-10,19,20-triazatetracyclo | |
| 209 | [13.5.2.1^{2,6}.0^{18,21}]tricosa-1(20),2(23),3,5,15,17,21-heptaen-9-one | |
| | 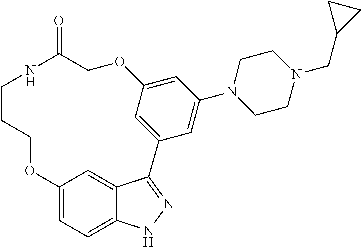 [SMILES: O=C1COc2cc(cc(N3CCN(CC4CC4)CC3)c2)-c2n[nH]c3ccc(cc23)OCCCN1] | LCMS method C: [M + H]⁺ = 462.3, t_{R} = 1.83 min LCMS method D: [M + H]⁺ = 462.2, t_{R} = 2.72 min ¹H NMR (400 MHz, DMSO) 13.04 (s, 1H), 8.54 (t, J = 6.0 Hz, 1H), 7.46 (d, J = 9.0 Hz, 1H), 7.34 (s, 1H), 7.19 (s, 1H), 6.97 (dd, J = 9.0, 2.1 Hz, 1H), 6.81 (s, 1H), 6.49 (s, 1H), 4.68 (s, 2H), 4.38-4.24 (m, 2H), 3.21 (brs, 7H), 2.59 (brs, 4H), 2.24 (brs, 2H), 1.97 (brs, 2H), 0.49 (d, J = 7.0 Hz, 2H), 0.11 (s, 2H) ppm. |
| Example | 4-(5,6-dihydro-2H-pyran-3-yl)-7,14-dioxa-10,19,20-triazatetracyclo | |
| 210 | [13.5.2.1^{2,6}.0^{18,21}]tricosa-1(20),2(23),3,5,15,17,21-heptaen-9-one | |
| | 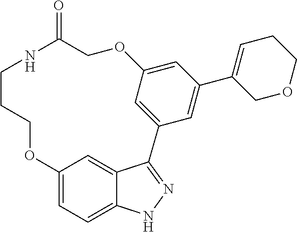 [SMILES: O=C1COc2cc(C3=CCCOC3)cc(c2)-c2n[nH]c3ccc(cc23)OCCCN1] | LCMS method E: [M + H]⁺ = 406.2, t_{R} = 2.51 min LCMS method D: [M + H]⁺ = 406.1, t_{R} = 2.89 min ¹H NMR (400 MHz, DMSO) 13.13 (s, 1H), 8.54 (t, J = 6.0 Hz, 1H), 7.58 (t, J = 1.4 Hz, 1H), 7.47 (d, J = 9.0 Hz, 1H), 7.33 (d, J = 1.8 Hz, 1H), 7.26-7.23 (m, 1H), 6.98 (dd, J = 9.0, 2.1 Hz, 1H), 6.90 (t, J = 2.0 Hz, 1H), 6.37-6.31 (m, 1H), 4.75 (s, 2H), 4.44 (q, J = 2.3 Hz, 2H), 4.38-4.29 (m, 2H), 3.75 (t, J = 5.5 Hz, 2H), 3.26-3.18 (m, 2H), 2.30-2.23 (m, 2H), 2.03-1.92 (m, 2H) ppm. |
| Example | 4-(2,5-dihydrofuran-3-yl)-7,14-dioxa-10,19,20-triazatetracyclo | |
| 211 | [13.5.2.1^{2,6}.0^{18,21}]tricosa-1(20),2(23),3,5,15,17,21-heptaen-9-one | |
| | 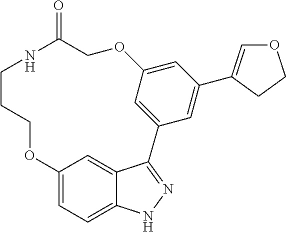 [SMILES: O=C1COc2cc(C3=COCC3)cc(c2)-c2n[nH]c3ccc(cc23)OCCCN1] | LCMS method C: [M + H]⁺ = 392.1, t_{R} = 2.52 min LCMS method D: [M + H]⁺ = 392.2, t_{R} = 2.93 min ¹H NMR (400 MHz, DMSO) 13.15 (s, 1H), 8.55 (t, J = 5.6 Hz, 1H), 7.61 (s, 1H), 7.49 (d, J = 9.0 Hz, 1H), 7.34 (s, 1H), 7.28 (s, 1H), 7.02 (s, 1H), 6.98 (d, J = 1.6 Hz, 1H), 6.58 (t, J = 2 Hz, 1H), 4.94 (brs, 2H), 4.77 (s, 2H), 4.75 (d, 2H), 4.35 (t, 2H), 3.23 (brs, 2H), 1.99 (brs, 2H) ppm. |
| Example | 4-(piperazin-1-yl)-7,14-dioxa-10,19,20-triazatetracyclo [13.5.2.1^{2,6}.0^{18,21}]tricosa- | |
| 212 | 1(20),2(23),3,5,15,17,21-heptaen-9-one | |
| | 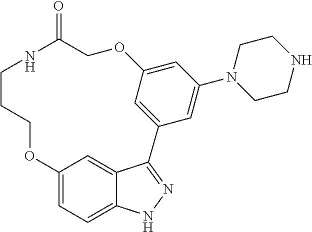 [SMILES: O=C1COc2cc(cc(N3CCNCC3)c2)-c2n[nH]c3ccc(cc23)OCCCN1] | LCMS method E: [M + H]⁺ = 408.1, t_{R} = 1.51 min ¹H NMR (400 MHz, DMSO) 13.06 (s, 1H), 8.55 (t, J = 6.0 Hz, 1H), 7.47 (d, J = 9.0 Hz, 1H), 7.35 (d, J = 1.9 Hz, 1H), 7.19 (d, J = 1.2 Hz, 1H), 6.97 (dd, J = 9.0, 2.1 Hz, 1H), 6.81 (s, 1H), 6.47 (t, J = 2.1 Hz, 1H), 4.69 (s, 2H), 4.34 (t, J = 7.37 Hz, 2H), 3.28-3.17 (m, 2H), 3.14-3.05 (m, 4H), 2.89-2.80 (m, 4H), 2.06- 1.88 (m, 2H) ppm. |
| Example | 4-(furan-3-yl)-7,14-dioxa-10,19,20-triazatetracyclo[13.5.2.1^{2,6}.0^{18,21}]tricosa- | |
| 213 | 1(20),2(23),3,5,15,17,21-heptaen-9-one | |
| | 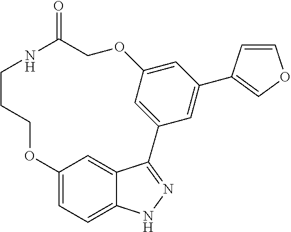 [SMILES: O=C1COc2cc(-c3ccoc3)cc(c2)-c2n[nH]c3ccc(cc23)OCCCN1] | LCMS method C: [M + H]⁺ = 390.1, t_{R} = 2.84 min ¹H NMR (400 MHz, DMSO) 13.15 (s, 1H), 8.56 (t, J = 5.9 Hz, 1H), 8.28 (s, 1H), 7.80 (s, 1H), 7.76 (t, J = 1.5 Hz, 1H), 7.49 (d, J = 9.0 Hz, 1H), 7.36 (s, 1H), 7.24 (s, 1H), 7.20 (t, 1H), 7.01 (s, 1H), 6.98 (d, J = 2.0 Hz, 1H), 4.78 (s, 2H), 4.35 (t, 2H), 3.24 (brs, 2H), 1.98 (brs, 2H) ppm. |
| Example | 4-(oxolan-3-yl)-7,14-dioxa-10,19,20-triazatetracyclo[13.5.2.1^{2,6}.0^{18,21}]tricosa- | |
| 214 | 1(20),2(23),3,5,15,17,21-heptaen-9-one | |
| | 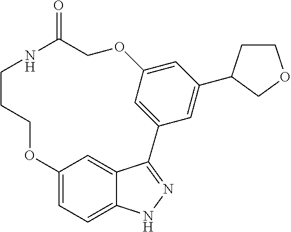 [SMILES: O=C1COc2cc(cc(C3CCOC3)c2)-c2n[nH]c3ccc(cc23)OCCCN1] | LCMS method C: [M + H]⁺ = 394.2, t_{R} = 2.44 min LCMS method D: [M + H]⁺ = 394.1, t_{R} = 2.78 min ¹H NMR (400 MHz, DMSO) 13.11 (s, 1H), 8.55 (t, J = 6.0 Hz, 1H), 7.53 (s, 1H), 7.48 (d, J = 9.0 Hz, 1H), 7.34 (s, 1H), 7.19 (s, 1H), 6.98 (dd, J = 9.0, 2.1 Hz, 1H), 6.85 (t, J = 1.8 Hz, 1H), 4.73 (s, 2H), 4.39-4.30 (m, 2H), 4.06 (t, J = 7.8 Hz, 1H), 3.97 (td, J = 8.3, 4.5 Hz, 1H), 3.81 (q, J = 15.6, 7.7 Hz, 1H), 3.61 (t, 1H), 3.48-3.38 (m, J = 15.4, 7.7 Hz, 2H), 3.23 (brs, 2H), 2.41- 2.28 (m, 1H), 2.04-1.90 (m, J = 12.2, 7.9 Hz, 2H) ppm. |
| Example | (11R,12R)-11,12-dimethyl-7,14-dioxa-10,19,20-triazatetracyclo | |
| 215 | [13.5.2.1^{2,6}.0^{18,21}]tricosa-1(20),2,4,6(23),15(22),16,18(21)-heptaen-9-one | |
| | 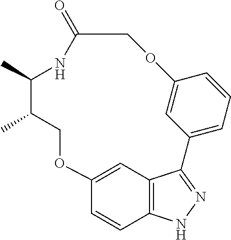 [SMILES: C[C@H]1COc2ccc3[nH]nc(c3c2)-c2cccc(c2)OCC(=O)N[C@@H]1C] | LCMS method E: [M + H]⁺ = 352.0, t_{R} = 2.59 min LCMS method D: [M + H]⁺ = 352.1, t_{R} = 2.93 min ¹H NMR (400 MHz, DMSO) 13.12 (s, J = 876.1 Hz, 1H), 8.49 (d, J = 7.9 Hz, 1H), 7.65 (d, J = 7.7 Hz, 1H), 7.48 (d, J = 9.0 Hz, 1H), 7.39 (s, 1H), 7.36 (d, J = 8.0 Hz, 1H), 7.32 (s, 1H), 7.02 (dd, J = 9.0, 2.0 Hz, 1H), 6.92 (dd, J = 8.2, 1.6 Hz, 1H), 4.76 (q, J = 16.0 Hz, 2H), 4.57 (d, J = 11.4 Hz, 1H), 4.06-3.89 (m, 2H), 2.61-2.51 (m, 2H), 1.06 (d, J = 7.3 Hz, 3H), 1.01 (d, J = 6.8 Hz, 3H) ppm. |
| Example | 4-[(3R,4R)-3,4-difluoropyrrolidin-1-yl]-7,14-dioxa-10,19,20-triazatetracyclo | |
| 216 | [13.5.2.1^{2,6}.0^{18,21}]tricosa-1(20),2(23),3,5,15,17,21-heptaen-9-one | |
| | 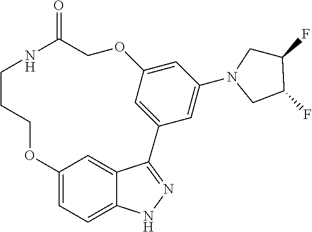 [SMILES: O=C1COc2cc(cc(N3C[C@@H](F)[C@H](F)C3)c2)-c2n[nH]c3ccc(cc23)OCCCN1] | LCMS method E: [M + H]⁺ = 429.1, t_{R} = 2.61 min LCMS method D: [M + H]⁺ = 429.1, t_{R} = 2.98 min ¹H NMR (400 MHz, DMSO) 13.04 (s, 1H), 8.53 (t, J = 6.0 Hz, 1H), 7.47 (d, J = 9.0 Hz, 1H), 7.36 (d, J = 1.6 Hz, 1H), 6.97 (dd, J = 9.0, 2.1 Hz, 1H), 6.88 (d, J = 1.0 Hz, 1H), 6.74 (s, 1H), 6.21 (t, J = 2.1 Hz, 1H), 5.58-5.51 (m, 1H), 5.46-5.39 (m, 1H), 4.69 (s, 2H), 4.33 (t, J = 7.9 Hz, 2H), 3.80-3.73 (m, 1H), 3.71-3.64 (m, 2H), 3.58 (dd, J = 23.3, 11.0 Hz, 1H), 3.27-3.18 (m, 2H), 1.97 (q, J = 10.1 Hz, 2H) ppm. |
| Example | 4-(4-cyclopropylpiperazin-1-yl)-7,14-dioxa-10,19,20-triazatetracyclo | |
| 217 | [13.5.2.1^{2,6}.0^{18,21}]tricosa-1(20),2(23),3,5,15,17,21-heptaen-9-one | |
| | 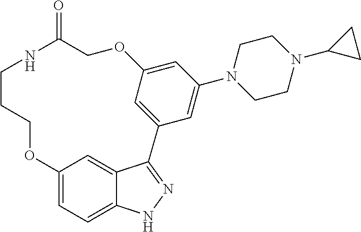 [SMILES: O=C1COc2cc(cc(N3CCN(C4CC4)CC3)c2)-c2n[nH]c3ccc(cc23)OCCCN1] | LCMS method E: [M + H]⁺ = 448.2, t_{R} = 1.60 min LCMS method D: [M + H]⁺ = 448.3, t_{R} = 2.94 min ¹H NMR (400 MHz, DMSO) 11.66 (s, 1H), 8.55 (t, J = 5.7 Hz, 1H), 7.47 (d, J = 9.0 Hz, 1H), 7.34 (d, J = 1.9 Hz, 1H), 7.19 (d, J = 1.3 Hz, 1H), 6.96 (dd, J = 9.0, 2.2 Hz, 1H), 6.80 (s, 1H), 6.47 (t, J = 2.1 Hz, 1H), 4.68 (s, 2H), 4.33 (t, J = 8.36 Hz, 2H), 3.23 (s, 2H), 3.18-3.10 (m, 4H), 2.74-2.64 (m, 4H), 2.03-1.92 (m, 2H), 1.70-1.62 (m, 1H), 0.48-0.41 (m, 2H), 0.38-0.31 (m, 2H) ppm. |
| Example | 4-(oxan-2-yl)-7,14-dioxa-10,19,20-triazatetracyclo[13.5.2.1^{2,6}.0^{18,21}]tricosa- | |
| 218 | 1(20),2(23),3,5,15,17,21-heptaen-9-one | |
| | 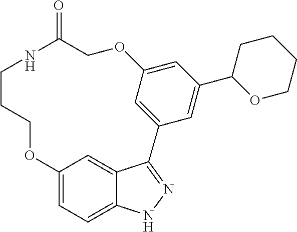 [SMILES: O=C1COc2cc(cc(C3CCCCO3)c2)-c2n[nH]c3ccc(cc23)OCCCN1] | LCMS method E: [M + H]⁺ = 408.1, t_{R} = 2.68 min LCMS method D: [M + H]⁺ = 408.1, t_{R} = 3.06 min ¹H NMR (400 MHz, DMSO) 8.57 (t, J = 5.8 Hz, 1H), 7.62 (s, 1H), 7.49 (d, J = 9.0 Hz, 1H), 7.35 (d, J = 2.0 Hz, 1H), 7.22 (s, 1H), 6.99 (dd, J = 9.0, 2.2 Hz, 1H), 6.86 (s, 1H), 4.75 (s, 2H), 4.41-4.30 (m, 3H), 4.06 (d, J = 11.0 Hz, 1H), 3.61-3.51 (m, 1H), 3.27-3.20 (m, 2H), 2.05-1.94 (m, 2H), 1.92-1.81 (m, 2H), 1.73- 1.53 (m, 3H), 1.51-1.38 (m, 1H) ppm. |
| Example | 4-fluoro-14-oxa-10,19,20-triazatetracyclo[13.5.2.1^{2,6}.0^{18,21}]tricosa- | |
| 219 | 1(20),2(23),3,5,15(22),16,18(21)-heptaen-9-one | |
| | 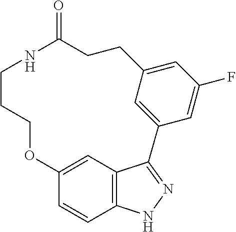 [SMILES: O=C1CCc2cc(F)cc(c2)-c2n[nH]c3ccc(cc23)OCCCN1] | LCMS method F: [M + H]⁺ = 340.2, t_{R} = 2.05 min LCMS method G: [M + H]⁺ = 340.2, t_{R} = 2.06 min ¹H NMR (400 MHz, DMSO) 13.20 (1H, s), 8.35 (1H, t, J = 5.5 Hz), 7.51-7.43 (3H, m), 7.23 (1H, d, J = 1.9 Hz), 7.09-6.98 (2H, m), 4.33-4.28 (2H, m), 3.21-3.19 (2H, m), 3.08-3.03 (2H, m), 2.63-2.59 (2H, m), 2.01-1.97 (2H, m) ppm. |
| Example | 14-oxa-4,10,19,20-tetraazatetracyclo[13.5.2.1^{2,6}.0^{18,21}]tricosa- | |
| 220 | 1(20),2(23),3,5,15(22),16,18(21)-heptaen-9-one | |
| | 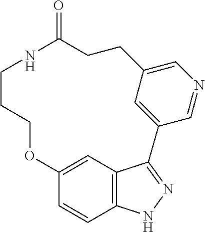 [SMILES: O=C1CCc2cncc(c2)-c2n[nH]c3ccc(cc23)OCCCN1] | LCMS method F: [M + H]⁺ = 323.2, t_{R} = 1.25 min LCMS method G: [M + H]⁺ = 323.2, t_{R} = 1.66 min ¹H NMR (400 MHz, DMSO) 13.26 (1H, s), 8.91 (1H, d, J = 2.3 Hz), 8.44-8.36 (2H, m), 7.95-7.93 (1H, m), 7.54-7.51 (1H, m), 7.18 (1H, d, J = 1.9 Hz), 7.01 (1H, dd, J = 2.2, 9.0 Hz), 4.30 (2H, dd, J = 7.7, 8.8 Hz), 3.20 (2H, s), 3.07-3.04 (2H, m), 2.68-2.60 (2H, m), 2.00-1.91 (2H, m) ppm. |
| Example | 11-methyl-7,14-dioxa-11,19,20-triazatetracyclo[13.5.2.1^{2,6}.0^{18,21}] tricosa- | |
| 221 | 1(20),2(23),3,5,15,17,21-heptaen-10-one | |
| | 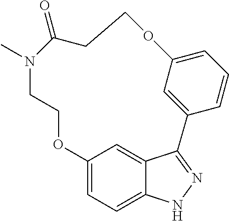 [SMILES: CN1CCOc2ccc3[nH]nc(c3c2)-c2cccc(c2)OCCC1=O] | LCMS method E: [M + H]⁺ = 338.1, t_{R} = 2.66 min LCMS method D: [M + H]⁺ = 338.1, t_{R} = 3.08 min ¹H NMR (400 MHz, DMSO, 100° C.) 12.82 (s, 1H), 7.62-7.44 (m, 2H), 7.36 (dd, J = 31.7, 23.9 Hz, 3H), 7.06 (d, J = 8.7 Hz, 1H), 6.94-6.85 (m, 1H), 4.60 (t, J = 6.7 Hz, 2H), 4.37 (t, J = 5.3 Hz, 2H), 3.75 (s, 2H), 2.94 (s, 3H), 2.09 (s, 2H) ppm. |
| Example | 8,14-dioxa-4,5,11,19,20,23-hexaazatetracyclo[13.5.2.1^{2,5}.0^{18,21}]tricosa- | |
| 222 | 1(20),2(23),3,15(22),16,18(21)-hexaen-10-one | |
| | 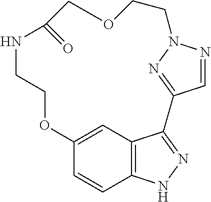 [SMILES: O=C1COCCn2ncc(n2)-c2n[nH]c3ccc(cc23)OCCN1] | LCMS method F: [M + H]⁺ = 329.2, t_{R} = 1.52 min LCMS method G: [M + H]⁺ = 329.2, t_{R} = 1.49 min ¹H NMR (400 MHz, DMSO) 13.15 (1H, s), 8.35-8.31 (1H, m), 8.12 (1H, s), 7.79 (1H, d, J = 2.3 Hz), 7.48-7.45 (1H, m), 7.04 (1H, dd, J = 2.4, 8.8 Hz), 4.81-4.77 (2H, m), 4.30 (2H, dd, J = 3.5, 4.8 Hz), 4.03 (2H, s), 4.01-3.98 (2H, m), 3.46 (2H, dd, J = 5.6, 8.8 Hz) ppm. |
| Example | (12R)-12-methyl-13-oxa-4,5,9,18,19,22-hexaazatetracyclo | |
| 223 | [12.5.2.1^{2,5}.0^{17,20}]docosa-1(19),2(22),3,14(21),15,17(20)-hexaen-8-one | |
| | 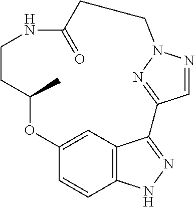 | LCMS method F: [M + H]⁺ = 327.3, t_{R} = 1.65 min LCMS method G: [M + H]⁺ = 327.3, t_{R} = 1.65 min ¹H NMR (400 MHz, DMSO) 13.05 (1H, s), 8.53-8.47 (1H, m), 8.12 (1H, s), 7.82-7.79 (1H, m), 7.45-7.41 (1H, d, J = 9.0 Hz), 6.97-6.93 (1H, dd, J = 2.5, 9.0 Hz), 4.86-4.77 (1H, m), 4.67-4.55 (2H, m), 3.89-3.80 (1H, m), 3.09-2.99 (1H, m), 2.86-2.71 (2H, m), 2.37-2.27 (1H, m), 1.45-1.41 (3H, d, J-6.12 Hz), 1.41-1.34 (1H, m) ppm. |
| Example | (12R)-12-methyl-13-oxa-4,5,9,18,19,22-hexaazatetracyclo | |
| 224 | [12.5.2.1^{2,5}.0^{17,20}]docosa-1(19),2(22),3,14(21),15,17(20)-hexaen-8-one | |
| | 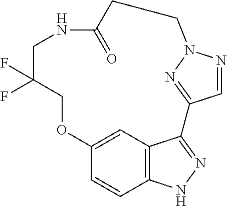 [SMILES: O=C1CCCn2ncc(n2)-c2n[nH]c3ccc(cc23)OCC(F)(F)CN1] | LCMS method F: [M + H]⁺ = 349.1, t_{R} = 1.78 min LCMS method G: [M + H]⁺ = 349.3, t_{R} = 1.63 min ¹H NMR (400 MHz, DMSO) 13.05 (1H, s), 8.53-8.47 (1H, m), 8.12 (1H, s), 7.82-7.79 (1H, m), 7.45-7.41 (1H, d, J = 9.0 Hz), 6.97-6.93 (1H, dd, J = 2.5, 9.0 Hz), 4.86-4.77 (1H, m), 4.67-4.55 (2H, m), 3.89-3.80 (1H, m), 3.09-2.99 (1H, m), 2.86-2.71 (2H, m), 2.37-2.27 (1H, m), 1.45-1.41 (3H, d, J = 6.12 Hz), 1.41-1.34 (1H, m) ppm. |
| Example | (11S)-11-fluoro-13-oxa-4,5,9,18,19,22-hexaazatetracyclo [12.5.2.1^{2,5}.0^{17,20}]docosa- | |
| 225 | 1(19),2(22),3,14(21),15,17(20)-hexaen-8-one | |
| | 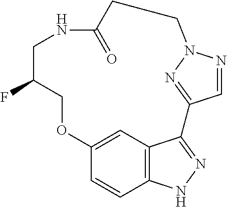 [SMILES: O=C1CCCn2ncc(n2)-c2n[nH]c3ccc(cc23)OC[C@@H](F)CN1] | LCMS method F: [M + H]⁺ = 331.2, t_{R} = 1.59 min LCMS method G: [M + H]⁺ = 331.3, t_{R} = 1.59 min ¹H NMR (400 MHz, DMSO) 13.15 (1H, s), 8.60 (1H, t, J-6.0 Hz), 8.14 (1H, s), 7.68 (1H, d, J = 2.3 Hz), 7.51 (1H, d, J = 9 Hz), 7.07 (1H, dd, J = 2.5, 9.1 Hz), 5.2-5.02 (1H, m), 4.81 (1H, m), 4.70-4.63 (2H, m), 4.37 (1H, m), 3.89 (1H, m), 3.06-2.98 (2H, m), 2.83 (1H, m) ppm. |
| Example | (12S)-12-methyl-13-oxa-4,5,9,18,19,22-hexaazatetracyclo [12.5.2.1^{2,5}.0^{17,20}]docosa- | |
| 226 | 1(19),2(22),3,14(21),15,17(20)-hexaen-8-one | |
| | 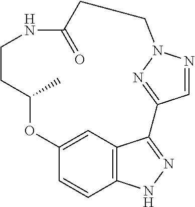 | LCMS method F: [M + H]⁺ = 327.3, t_{R} = 1.65 min LCMS method G: [M + H]⁺ = 327.3, t_{R} = 1.65 min ¹H NMR (400 MHz, DMSO) 13.04 (1H, s), 8.53-8.47 (1H, m), 8.12 (1H, s), 7.82-7.79 (1H, m), 7.45-7.41 (1H, d, J-9.0 Hz), 6.98-6.93 (1H, dd, J = 2.4, 8.9 Hz), 4.86-4.77 (1H, m), 4.67-4.55 (2H, m), 3.90-3.79 (1H, m), 3.09-2.99 (1H, m), 2.87-2.71 (2H, m), 2.37-2.27 (1H, m), 1.46-1.41 (3H, d), 1.40-1.33 (1H, m) ppm. |
| Example | (11R)-11-fluoro-13-oxa-4,5,9,18,19,22-hexaazatetracyclo [12.5.2.1^{2,5}.0^{17,20}]docosa- | |
| 227 | 1(19),2(22),3,14(21),15,17(20)-hexaen-8-one | |
| | 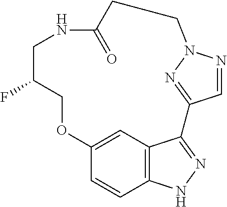 [SMILES: O=C1CCCn2ncc(n2)-c2n[nH]c3ccc(cc23)OC[C@H](F)CN1] | LCMS method F: [M + H]⁺ = 331.2, t_{R} = 1.59 min LCMS method G: [M + H]⁺ = 331.3, t_{R} = 1.59 min ¹H NMR (400 MHz, DMSO) 13.15 (1H, s), 8.59 (1H, t, J = 6.4 Hz), 8.14 (1H, s), 7.67 (1H, d, J-2.1 Hz), 7.51 (1H, d, J = 8.9 Hz), 7.07 (1H, dd, J = 2.6, 9.0 Hz), 5.19-5.02 (1H, m), 4.85 4.78 (1H, m), 4.69-4.54 (2H, m), 4.43-4.31 (1H, m), 3.90 (1H, m), 3.06-2.98 (2H, m), 2.86-2.81 (1H, m) ppm. |
| Example | (11R,12S)-11,12-dimethyl-7,14-dioxa-10,19,20-triazatetracyclo | |
| 228 | [13.5.2.1^{2,6}.0^{18,21}]tricosa-1(20),2,4,6(23),15(22),16,18(21)-heptaene | |
| | 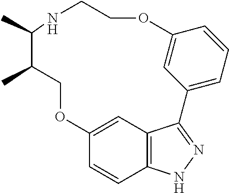 [SMILES: C[C@@H]1COc2ccc3[nH]nc(c3c2)-c2cccc(c2)OCCN[C@@H]1C] | LCMS method E: [M + H]⁺ = 338.1, t_{R} = 1.81 min LCMS method D: [M + H]⁺ = 338.1, t_{R} = 3.75 min ¹H NMR (400 MHz, DMSO) 13.04 (s, 1H), 7.91 (s, 1H), 7.81 (d, J = 1.7 Hz, 1H), 7.52 (d, J = 7.7 Hz, 1H), 7.46 (d, J = 9.0 Hz, 1H), 7.34 (t, J = 7.9 Hz, 1H), 7.00 (dd, J = 9.0, 2.2 Hz, 1H), 6.88-6.80 (m, 1H), 4.69 (d, J = 12.0 Hz, 1H), 4.43-4.25 (m, 2H), 3.90-3.80 (m, 1H), 3.15-3.05 (m, 1H), 3.02-2.90 (m, 1H), 2.76- 2.64 (m, 1H), 2.38-2.28 (m, 1H), 2.22-2.09 (m, 1H), 1.02-0.91 (m, 6H) ppm. |
| Example | (8R or 8S,13S)-8,13-dimethyl-7,14-dioxa-4,10,19,20-tetraazatetracyclo | |
| 229 | [13.5.2.1^{2,6}.0^{18,21}]tricosa-1(20),2,4,6(23),15(22),16,18(21)-heptaene | |
| | 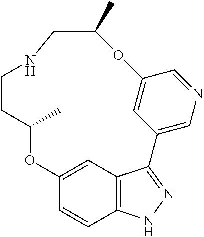 [SMILES: C[C@@H]1CNCC[C@H](C)Oc2ccc3[nH]nc(c3c2)-c2cncc(c2)O1] | Absolute stereochemistry unknown LCMS method E: [M + H]⁺ = 339.1, t_{R} = 1.50 min LCMS method D: [M + H]⁺ = 339.1, t_{R} = 3.56 min ¹H NMR (400 MHz, CDCl₃) 10.37 (s, 1H), 8.89 (d, J = 1.6 Hz, 1H), 8.48-8.39 (m, 1H), 8.30 (d, J = 2.7 Hz, 1H), 8.15 (d, J = 2.1 Hz, 1H), 7.40 (d, J = 9.0 Hz, 1H), 7.07 (dd, J = 9.0, 2.2 Hz, 1H), 4.70 (ddd, J = 13.5, 8.2, 5.0 Hz, 2H), 3.26 (dd, J = 12.5, 7.2 Hz, 1H), 3.13 (ddd, J = 12.3, 5.4, 2.3 Hz, 1H), 2.72 (dd, J = 12.5, 3.6 Hz, 1H), 2.58-2.45 (m, 1H), 2.21 (ddt, J = 14.0, 11.2, 2.7 Hz, 1H), 1.62-1.53 (m, 1H), 1.46 (s, 3H), 1.45 (s, 3H) ppm. |
| Example | (8S or 8R,13S)-8,13-dimethyl-7,14-dioxa-4,10,19,20-tetraazatetracyclo | |
| 230 | [13.5.2.1^{2,6}.0^{18,21}]tricosa-1(20),2,4,6(23),15(22),16,18(21)-heptaene | |
| | 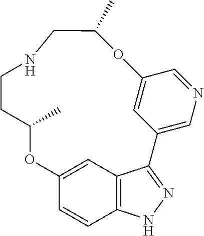 [SMILES: C[C@H]1CCNC[C@H](C)Oc2cncc(c2)-c2n[nH]c3ccc(cc23)O1] | Absolute stereochemistry unknown LCMS method E: [M + H]⁺ = 339.1, t_{R} = 1.45 min LCMS method D: [M + H]⁺ = 339.1, t_{R} = 3.11 min ¹H NMR (400 MHz, CDCl₃) 10.20 (s, 1H), 8.80 (d, J = 1.7 Hz, 1H), 8.59-8.53 (m, 1H), 8.32 (d, J = 2.7 Hz, 1H), 7.80 (d, J = 2.1 Hz, 1H), 7.41 (d, J = 9.0 Hz, 1H), 7.06 (dd, J = 9.0, 2.3 Hz, 1H), 4.46 (dq, J = 12.2, 6.1 Hz, 1H), 4.36 (pd, J = 6.6, 2.5 Hz, 1H), 3.19 (dd, J = 13.2, 7.6 Hz, 1H), 2.93-2.78 (m, 2H), 2.73 (ddd, J = 12.4, 9.9, 2.2 Hz, 1H), 2.27-2.14 (m, 1H), 1.81-1.69 (m, 2H), 1.43 (s, 3H), 1.42 (s, 3H) ppm. |
| Example | (11R,12R)-12-fluoro-11-methyl-7,14-dioxa-10,19,20-triazatetracyclo | |
| 231 | [13.5.2.1^{2,6}.0^{18,21}]tricosa-1(20),2,4,6(23),15(22),16,18(21)-heptaene | |
| | 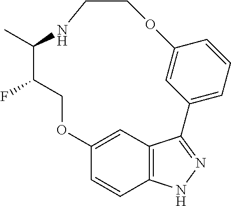 [SMILES: C[C@H]1NCCOc2cccc(c2)-c2n[nH]c3ccc(cc23)OC[C@@H]1F] | LCMS method C: [M + H]⁺ = 342.2, t_{R} = 1.98 min ¹H NMR (400 MHz, DMSO) 13.05 (s, 1H), 8.13 (d, J = 11.2 Hz, 2H), 7.57 (d, J = 7.6 Hz, 1H), 7.49 (d, J = 9.0 Hz, 1H), 7.32 (t, J = 7.9 Hz, 1H), 7.05 (dd, J = 9.0, 2.0 Hz, 1H), 6.85 (dd, J = 8.1, 1.7 Hz, 1H), 4.85-4.76 (m, 2H), 4.58-4.47 (m, 1H), 4.42- 4.24 (m, 2H), 3.40 (t, J = 10.8 Hz, 1H), 2.87-2.72 (m, 1H), 2.72- 2.60 (m, 1H), 2.25-2.02 (m, 1H), 1.16 (dd, J = 6.2, 3.3 Hz, 3H) ppm. |
| Example | (13S)-13-methyl-7,14-dioxa-4,10,19,20-tetraazatetracyclo | |
| 232 | [13.5.2.1^{2,6}.0^{18,21}]tricosa-1(20),2,4,6(23),15(22),16,18(21)-heptaene | |
| | 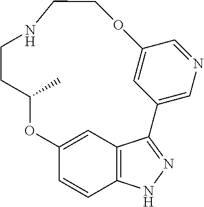 [SMILES: C[C@H]1CCNCCOc2cncc(c2)-c2n[nH]c3ccc(cc23)O1] | LCMS method E: [M + H]⁺ = 325.1, t_{R} = 1.39 min LCMS method D: [M + H]⁺ = 325.1, t_{R} = 3.26 min ¹H NMR (400 MHz, DMSO) 13.18 (s, 1H), 8.70 (s, 1H), 8.57 (s, 1H), 8.18-8.14 (m, 2H), 7.48 (d, J = 9.0 Hz, 1H), 6.98 (dd, J = 9.0, 2.1 Hz, 1H), 4.68-4.58 (m, 1H), 4.50-4.34 (m, 2H), 3.12- 3.02 (m, J = 15.4, 6.6 Hz, 1H), 2.91-2.80 (m, 2H), 2.45 (m 1H), 2.15 (t, 1H), 1.57-1.44 (m, 1H), 1.38 (d, J = 6.1 Hz, 3H) ppm. |
| Example | (12S)-12-methyl-13-oxa-4,5,9,18,19-pentaazatetracyclo [12.5.2.1^{2,5}.0^{17,20}] docosa- | |
| 233 | 1(19),2(22),3,14(21),15,17(20)-hexaene | |
| | 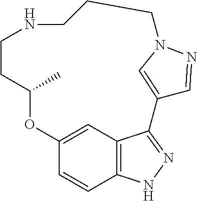 [SMILES: C[C@H]1CCNCCCn2cc(cn2)-c2n[nH]c3ccc(cc23)O1] | LCMS method J: [M + H]⁺ = 312.3, t_{R} = 1.89 min LCMS method G: [M + H]⁺ = 312.3, t_{R} = 1.93 min ¹H NMR (400 MHz, DMSO) 12.64 (1H, s), 8.88 (1H, s), 7.66 (1H, s), 7.61 (1H, m), 7.37-7.35 (1H, d), 6.92-6.90 (1H, dd, J = 2.4, 8.8 Hz), 4.63-4.56 (1H, m), 4.43-4.36 (1H, m), 4.30-4.23 (1H, m), 2.74-2.60 (3H, m), 2.22-2.06 (2H, m), 2.05-1.96 (2H, m), 1.46-1.39 (1H, m), 1.36 (3H, d) ppm. |
| Example | (7S or 7R,13R)-7,13-dimethyl-8,14-dioxa-4,11,19,20-tetraazatetracyclo | |
| 234 | [13.5.2.1^{2,6}.0^{18,21}]tricosa-1(20),2(23),3,5,15(22),16,18(21)-heptaene | |
| | 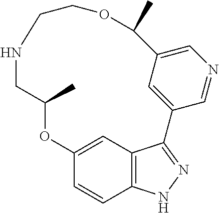 [SMILES: C[C@@H]1CNCCO[C@@H](C)c2cncc(c2)-c2n[nH]c3ccc(cc23)O1] | Absolute stereochemistry unknown LCMS method H: [M + H]⁺ = 339.3, t_{R} = 1.40 min LCMS method G: [M + H]⁺ = 339.3, t_{R} = 2.03 min ¹H NMR (400 MHz, MeOD) : 8.99 (1H, s), 8.64 (1H, t, J = 1.8 Hz), 8.52 (1H, m), 7.60 (1H, m), 7.57 (1H, d, J = 9 Hz), 7.18 (1H, dd, J-9 Hz, J = 2.2 Hz), 4.55 (1H, m), 4.03 (1H, m), 3.92 (1H, m), 3.73 (1H, m), 3.58 (1H, m), 3.17 (1H, m), 1.67 (d, J = 6.5 Hz, 3H), 1.56 (d, J = 5.9 Hz, 3H) ppm. |
| Example | (7R or 7S,13R)-7,13-dimethyl-8,14-dioxa-4,11,19,20-tetraazatetracyclo | |
| 235 | [13.5.2.1^{2,6}.0^{18,21}]tricosa-1(20),2(23),3,5,15(22),16,18(21)-heptaene | |
| | 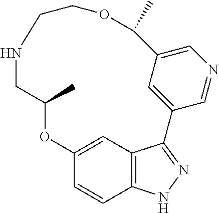 [SMILES: C[C@@H]1CNCCO[C@H](C)c2cncc(c2)-c2n[nH]c3ccc(cc23)O1] | Absolute stereochemistry unknown LCMS method F: [M + H]⁺ = 339.2, t_{R} = 0.99 min LCMS method G: [M + H]⁺ = 339.2, t_{R} = 2.27 min ¹H NMR (400 MHz, MeOD) 9.35 (1H, m), 9.14 (1H, m), 8.80 (1H, m), 7.63 (1H, d, J-9 Hz), 7.56 (1H, m), 7.19 (1H, m), 5.09 (1H, m), 4.85 (1H, m), 4.25 (1H, d, J = 10.4 Hz), 3.95 (1H, t, J = 10 Hz), 3.88 (1H, m), 3.72 (1H, m), 3.41 (1H, m), 3.34 (1H, m), 1.67 (3H, d, J = 3.1 Hz), 1.62 (3H, d, J = 3.4 Hz) ppm. |
| Example | 4-(morpholin-4-yl)-7,14-dioxa-11,19,20-triazatetracyclo [13.5.2.1^{2,6}.0^{18,21}]tricosa- | |
| 236 | 1(20),2,4,6(23),15,17,21-heptaene | |
| | 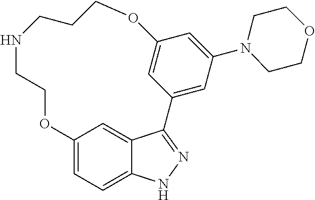 [SMILES: c1cc2[nH]nc3c2cc1OCCNCCCOc1cc-3cc(N2CCOCC2)c1] | LCMS method E: [M + H]⁺ = 395.1, t_{R} = 1.59 min LCMS method D: [M + H]⁺ = 395.1, t_{R} = 3.33 min ¹H NMR (300 MHz, DMSO) 12.99 (s, 1H), 8.39 (s, 1H), 7.43 (d, J = 9.0 Hz, 1H), 7.35 (s, 1H), 7.21 (s, 1H), 6.99 (d, J = 8.6 Hz, 1H), 6.42 (s, 1H), 4.36 (bb, 2H), 4.24 (bb, 2H), 3.75 (bb,4 H), 3.29 (bb, 1H), 3.14 (bb, 4H), 2.96 (bb, 2H), 2.67 (bb, 2H), 1.94 (bb, 2H) ppm. |
| Example | 7,14-dioxa-10,19,20,23-tetraazatetracyclo[13.5.2.1^{2,6}.0^{18,21}]tricosa- | |
| 237 | 1(20),2,4,6(23),15,17,21-heptaene | |
| | 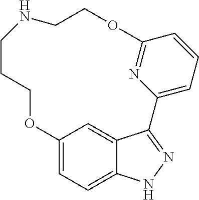 [SMILES: c1cc2nc(c1)-c1n[nH]c3ccc(cc13)OCCCNCCO2] | LCMS method E: [M + H]⁺ = 311.1, t_{R} = 1.68 min LCMS method D: [M + H]⁺ = 311.1, t_{R} = 3.35 min ¹H NMR (300 MHz, DMSO) 13.22 (s, 1H), 8.09 (s, 1H), 7.84- 7.69 (m, 2H), 7.48 (d, J = 9.0 Hz, 1H), 6.98 (d, J = 9.1 Hz, 1H), 6.70 (d, J = 7.6 Hz, 1H), 4.45 (dt, J = 7.8 Hz, 4H), 3.07 (t, J = 9.1 Hz, 2H), 2.78 (t, J = 5.4 Hz, 2H), 2.12 (s, 1H), 1.98-1.81 (m, 2H) ppm. |
| Example | 7,14-dioxa-4,11,19,20-tetraazatetracyclo[13.5.2.1^{2,6}.0^{18,21}]tricosa- | |
| 238 | 1(20),2,4,6(23),15,17,21-heptaene | |
| | 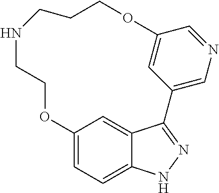 [SMILES: c1ncc2cc1OCCCNCCOc1ccc3[nH]nc-2c3c1] | LCMS method E: [M + H]⁺ = 311.1, t_{R} = 1.02 min LCMS method D: [M + H]⁺ = 311.1, t_{R} = 0.90 min ¹H NMR (400 MHz, DMSO) 13.25 (s, 1H), 8.81 (d, J = 1.5 Hz, 1H), 8.55 (d, J = 2.1 Hz, 1H), 8.28-8.21 (m, 1H), 8.18 (d, J = 2.7 Hz, 1H), 7.48 (d, J = 9.0 Hz, 1H), 7.03 (dd, J = 9.0, 2.2 Hz, 1H), 4.45 (t, J = 7.7 Hz, 2H), 4.27 (t, J = 4.9 Hz, 2H), 2.98 (t, J = 4.9 Hz, 2H), 2.67 (t, J = 5.7 Hz, 2H), 1.95 (dt, 2H) ppm. |
| Example | 10-methyl-4-(propan-2-yloxy)-7,14-dioxa-10,19,20-triazatetracyclo | |
| 239 | [13.5.2.1^{2,6}.0^{18,21}]tricosa-1(20),2,4,6(23),15,17,21-heptaene | |
| | 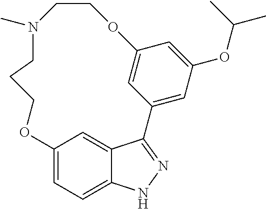 [SMILES: CC(C)Oc1cc2cc(c1)-c1n[nH]c3ccc(cc13)OCCCN(C)CCO2] | LCMS method C: [M + H]⁺ = 382.1, t_{R} = 2.14 min LCMS method D: [M + H]⁺ = 382.1, t_{R} = 4.17 min ¹H NMR (400 MHz, DMSO) 13.07 (s, 1H), 7.68 (s, 1H), 7.47 (d, J = 9.0 Hz, 1H), 7.39 (s, 1H), 7.07 (d, J = 1.3 Hz, 1H), 6.99 (dd, J = 9.0, 2.1 Hz, 1H), 6.40 (t, J = 2.2 Hz, 1H), 4.70-4.57 (m, J = 12.0, 6.0 Hz, 1H), 4.45-4.27 (m, 4H), 2.84-2.73 (m, 2H), 2.38-2.28 (m, 2H), 2.20 (s, 3H), 1.94 (s, 2H), 1.29 (d, J = 6.0 Hz, 6H) ppm. |
| Example | 11-methyl-7,14-dioxa-10,19,20,23-tetraazatetracyclo[13.5.2.1^{2,6}.0^{18,21}]tricosa- | |
| 240 | 1(20),2,4,6(23),15,17,21-heptaene | |
| | 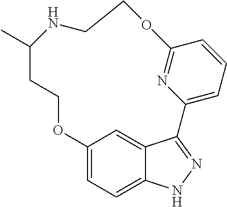 [SMILES: CC1CCOc2ccc3[nH]nc(c3c2)-c2cccc(n2)OCCN1] | LCMS method E: [M + H]⁺ = 325.1, t_{R} = 1.78 min LCMS method D: [M + H]⁺ = 325.1, t_{R} = 3.22 min ¹H NMR (400 MHz, DMSO) 13.22 (s, 1H), 8.11 (d, J = 2.2 Hz, 1H), 7.81-7.74 (m, 2H), 7.48 (d, J = 9.0 Hz, 1H), 6.98 (dd, J = 9.0, 2.4 Hz, 1H), 6.70 (dd, J = 7.5, 1.4 Hz, 1H), 4.51-4.40 (m, 4H), 3.12-3.04 (m, 2H), 3.02-2.95 (m, 1H), 1.95-1.83 (m, 2H), 1.82-1.74 (m, 1H), 1.09 (d, J = 6.6 Hz, 3H) ppm. |
| Example | 11,11-difluoro-13-oxa-4,5,9,18,19,22-hexaazatetracyclo [12.5.2.1^{2,5}.0^{17,20}] docosa- | |
| 241 | 1(19),2(22),3,14(21),15,17(20)-hexaen-8-one | |
| | 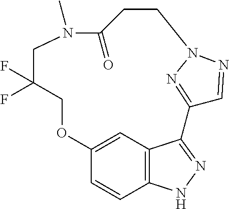 [SMILES: CN1CC(F)(F)COc2ccc3[nH]nc(c3c2)-c2cnn(n2)CCC1=O] | LCMS method F: [M + H]⁺ = 363.3, t_{R} = 1.80 min LCMS method G: [M + H]⁺ = 363.3, t_{R} = 1.77 min ¹H NMR (400 MHz, DMSO) 13.12 (1H, s), 8.74 (1H, t, J = 6.0 Hz), 8.14 (1H, s), 7.95 (1H, s), 7.47-7.44 (1H, m), 7.10 (1H, dd, J = 2.6, 9.0 Hz), 4.79-4.74 (4H, m),3.82-3.6 (2H, m), 2.96-2.94 (2H, m) ppm. |
| Example | (6S,12R)-6,12-dimethyl-18-(oxan-2-yl)-13-oxa-4,5,9,18,19,22-hexaazatetra | |
| 242 | cyclo[12.5.2.1^{2,5}.0^{17,20}]docosa-1(19),2(22),3,14(21), 15,17(20)-hexaen-8-one | |
| | 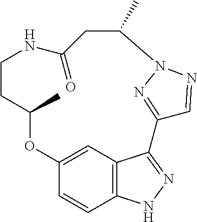 [SMILES: C[C@@H]1CCNC(=O)C[C@H](C)n2ncc(n2)-c2n[nH]c3ccc(cc23)O1] | LCMS method F: [M + H]⁺ = 341.2, t_{R} = 1.90 min LCMS method G: [M + H]⁺ = 341.3, t_{R} = 1.77 min ¹H NMR (400 MHz, DMSO) 13.04 (1H, s), 8.27-8.24 (1H, m), 8.13 (1H, s), 7.74 (1H, s), 7.45-7.42 (1H, m), 6.95 (1H, dd, J = 2.0, 8.6 Hz), 5.02-4.96 (1H, m), 4.61-4.53 (1H, m), 3.54-3.44 (2H, m), 3.08-2.99 (2H, m), 2.74 (1H, dd, J = 6.5, 16.8 Hz), 2.19-2.08 (1H, m), 1.86-1.82 (3H, m), 1.41 (3H, m) ppm. |
| Example | (6S,12R)-6,9,12-trimethyl-18-(oxan-2-yl)-13-oxa-4,5,9,18,19,22-hexaazatetra | |
| 243 | cyclo [12.5.2.1^{2,5}.0^{17,20}]docosa-1(19),2(22),3,14(21), 15,17(20)-hexaen-8-one | |
| | 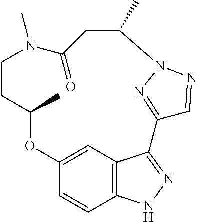 [SMILES: C[C@@H]1CCN(C)C(=O)C[C@H](C)n2ncc(n2)-c2n[nH]c3ccc(cc23)O1] | LCMS method F: [M + H]⁺ = 355.2, t_{R} = 2.07 min LCMS method G: [M + H]⁺ = 355.3, t_{R} = 2.01 min ¹H NMR (400 MHz, DMSO) 13.07-13.00 (1H, m), 8.12-8.08 (1H, m), 7.47-7.43 (2H, m), 6.98-6.93 (1H, m), 5.13-5.06 (1H, m), 4.53-4.46 (1H, m), 4.12-4.02 (1H, m), 2.90-2.88 (3H, m), 2.69-2.64 (1H, m), 1.94-1.88 (1H, m), 1.78 (3H, d, J = 6.8 Hz), 1.71-1.65 (1H, m), 1.56-1.48 (2H, m), 1.39 (3H, d, J = 5.9 Hz) ppm. |
| Example | (6S,12R)-6,9,12-trimethyl-13-oxa-4,5,9,18,19,22-hexaazatetracyclo | |
| 244 | [12.5.2.1^{2,5}.0^{17,20}]docosa-1(19),2(22),3,14(21),15,17(20)-hexaene | |
| | 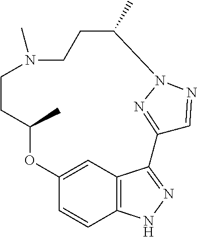 [SMILES: C[C@@H]1CCN(C)CC[C@H](C)n2ncc(n2)-c2n[nH]c3ccc(cc23)O1] | LCMS method F: [M + H]⁺ = 341.3, t_{R} = 1.29 min LCMS method G: [M + H]⁺ = 341.3, t_{R} = 2.445 min ¹H NMR (400 MHz, DMSO) 10.15 (1H, s), 8.14-8.07 (2H, m), 7.38-7.35 (1H, m), 7.07 (1H, dd, J = 2.5, 8.9 Hz), 5.05-4.97 (1H, m), 4.83-4.75 (1H, m), 3.74-3.65 (1H, m), 2.71-2.44 (4H, m), 2.36-2.32 (1H, m), 2.31 (3H, s), 1.65 (3H, d, J = 7.0 Hz), 1.46-1.44 (3H, m) ppm. |

### Example 245: 12R)-12-methyl-9,13-dioxa-4,5,15,18,19-pentaazatetracyclo[12.5.2.1^{2,5}. 0^{17,20}]docosa-1(19),2(22),3,14(21),15,17(20)-hexaene

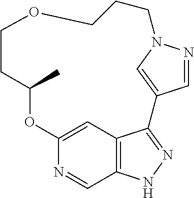 [SMILES: C[C@@H]1CCOCCCn2cc(cn2)-c2n[nH]c3cnc(cc23)O1]

Example 245 is prepared according to the synthesis route described below.

### Preparation of Intermediate 655: 2-[(1R)-3-(3-benzyloxypropoxy)-1-methyl-propoxy]-4-methyl-5-nitro-pyridine

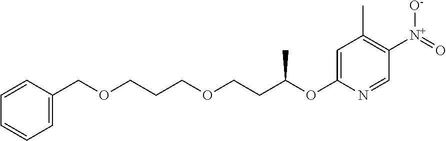 [SMILES: Cc1cc(O[C@H](C)CCOCCCOCc2ccccc2)ncc1[N+](=O)[O-]]

To a solution of 2-bromo-4-methyl-5-nitro-pyridine (4.40 g, 20.3 mmol) and (2R)-4-(3-benzyloxypropoxy)butan-2-ol (3.22 g, 13.53 mmol) in dry toluene (100 mL), purged with argon, was added bis(dibenzylideneacetone)palladium(0) (156 mg, 0.270 mmol), rac-BINAP (506 mg, 0.812 mmol) and cesium carbonate (6.173 g, 18.945 mmol). The reaction mixture was stirred at 110° C. After 18 hours, additional bis(dibenzylideneacetone)palladium(0) (78 mg, 0.135 mmol), rac-BINAP (253 mg, 0.406 mmol), cesium carbonate (3.086 g, 9.472 mmol) were added. The reaction mixture was stirred at 110° C. for 18 h. The reaction mixture was diluted with water and ethyl acetate. The phases were separated and the aqueous layer was extracted with ethyl acetate. The combined organic layers were washed with brine, dried over anhydrous sodium sulfate, filtered and evaporated under reduced pressure. The residue was purified by silica gel column chromatography using cyclohexane/ethyl acetate 100/0 to 90/10 as eluent to afford 2-[(1R)-3-(3-benzyloxypropoxy)-1-methyl-propoxy]-4-methyl-5-nitro-pyridine as a pale yellow oil.

LCMS method F: [M+H]⁺=375.2, t_{R}=3.24 min

### Preparation of Intermediate 656: 6-[(1R)-3-(3-benzyloxypropoxy)-1-methyl-propoxy]-4-methyl-pyridin-3-amine

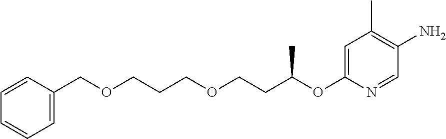 [SMILES: Cc1cc(O[C@H](C)CCOCCCOCc2ccccc2)ncc1N]

To a solution of 2-[(1R)-3-(3-benzyloxypropoxy)-1-methyl-propoxy]-4-methyl-5-nitro-pyridine (3.38 g, 9.02 mmol) in ethanol (72 mL) and water (18 mL) was added ammonium chloride (4.74 g, 90.27 mmol) and iron (5.05 g, 90.27 mmol) under argon atmosphere. The reaction mixture was stirred at 80° for 18 h. The reaction mixture was filtered over a Celite pad and evaporated under reduced pressure. Ethyl acetate and saturated aqueous sodium bicarbonate solution were added and the aqueous layer was extracted with ethyl acetate. The combined organic layers were dried over anhydrous sodium sulfate, filtered and evaporated under reduced pressure to afford 6-[(1R)-3-(3-benzyloxypropoxy)-1-methyl-propoxy]-4-methyl-pyridin-3-amine which was used in the next step without further purification.

LCMS method F: [M+H]⁺=345.2, t_{R}=2.00 min

### Preparation of Intermediate 657: N-[6-[(1R)-3-(3-benzyloxypropoxy)-1-methyl-propoxy]-4-methyl-3-pyridyl]acetamide

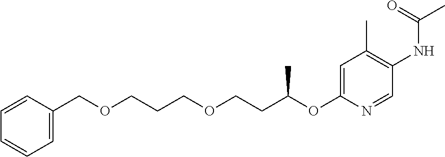 [SMILES: CC(=O)Nc1cnc(O[C@H](C)CCOCCCOCc2ccccc2)cc1C]

To a solution of 6-[(1R)-3-(3-benzyloxypropoxy)-1-methyl-propoxy]-4-methyl-pyridin-3-amine (2.99 g, 8.70 mmol) in dichloromethane (30 mL) was added triethylamine (3.6 mL, 26.11 mmol). The reaction mixture was cooled to 0° C. and anhydride acetic (1.5 mL, 15.66 mmol) was added. The reaction mixture was stirred at RT for 2 h. Saturated aqueous bicarbonate solution and dichloromethane were added. After separation, the aqueous layer was extracted with dichloromethane. The combined organic layer was washed with brine, dried over anhydrous sodium sulfate, filtered and evaporated under reduced pressure. The residue was purified by silica gel column chromatography using cyclohexane/ethyl acetate 100/0 to 0/100 as eluent to afford N-[6-[(1R)-3-(3-benzyloxypropoxy)-1-methyl-propoxy]-4-methyl-3-pyridyl]acetamide as an orange oil.

LCMS method F: [M+H]⁺=387.2, t_{R}=2.59 min

### Preparation of Intermediate 658: Mixture of 1-[5-[(1R)-3-(3-benzyloxypropoxy)-1-methyl-propoxy]pyrazolo[3,4-c]pyridin-1-yl]ethanone and 1-[5-[(1R)-3-(3-benzyloxypropoxy)-1-methyl-propoxy]pyrazolo[3,4-c]pyridin-2-yl]ethanone

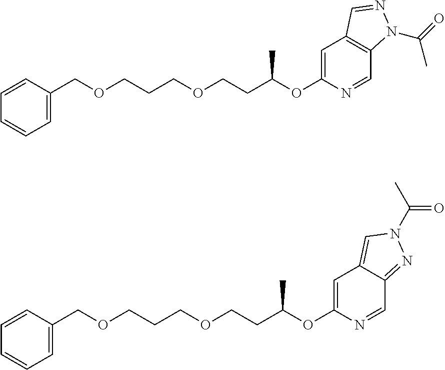 [SMILES: CC(=O)n1ncc2cc(O[C@H](C)CCOCCCOCc3ccccc3)ncc21 ; CC(=O)n1cc2cc(O[C@H](C)CCOCCCOCc3ccccc3)ncc2n1]

To a solution of N-[6-[(1R)-3-(3-benzyloxypropoxy)-1-methyl-propoxy]-4-methyl-3-pyridyl]acetamide (2.81 g, 7.27 mmol), potassium acetate (1.07 g, 10.91 mmol) and anhydride acetic (1.4 mL, 14.55 mmol) in dry toluene (55 mL) at 80° C. was added dropwise tert-butyl nitrite (5.2 mL, 43.65 mmol). The reaction mixture was stirred at 80° C. for 18 h. Ethyl acetate and saturated aqueous sodium bicarbonate solution were added and phases were separated. The aqueous layer was extracted with ethyl acetate and the combined organic layer was washed with brine, dried over anhydrous sodium sulfate, filtered and evaporated under reduced pressure to afford a mixture of 1-[5-[(1R)-3-(3-benzyloxypropoxy)-1-methyl-propoxy]pyrazolo[3,4-c]pyridin-1-yl]ethanone and 1-[5-[(1R)-3-(3-benzyloxypropoxy)-1-methyl-propoxy]pyrazolo [3,4-c]pyridin-2-yl]ethanone as an orange oil which was engaged in next step without further purification.

LCMS method F: [M+H]⁺=398.2, t_{R}=3.03 min and [M+H]⁺=398.2, t_{R}=3.15 min

### Preparation of Intermediate 659: 5-[(1R)-3-(3-benzyloxypropoxy)-1-methyl-propoxy]-1H-pyrazolo[3,4-c]pyridine

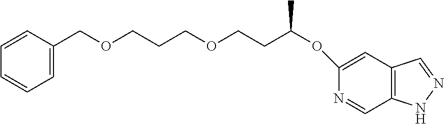 [SMILES: C[C@H](CCOCCCOCc1ccccc1)Oc1cc2cn[nH]c2cn1]

To a mixture of 1-[5-[(1R)-3-(3-benzyloxypropoxy)-1-methyl-propoxy]pyrazolo[3,4-c]pyridin-1-yl]ethenone and 1-[5-[(1R)-3-(3-benzyloxypropoxy)-1-methyl-propoxy]pyrazolo [3,4-c]pyridin-2-yl]ethanone (2.83 g, 7.12 mmol) in methanol (50 ml) was added ammonia (4M solution in methanol) (12.5 mL, 49.89 mmol). The reaction mixture was stirred at RT for 2 h. The reaction mixture was evaporated under reduced. The residue was purified by silica gel column chromatography using cyclohexane/ethyl acetate 100/0 to 60/40 as eluent to afford 5-[(1R)-3-(3-benzyloxypropoxy)-1-methyl-propoxy]-1H-pyrazolo[3,4-c]pyridine as an orange oil.

LCMS method F: [M+H]⁺=356.3, t_{R}=2.53 min

### Preparation of Intermediate 660: 5-[(1R)-3-(3-benzyloxypropoxy)-1-methyl-propoxy]-3-iodo-1H-pyrazolo[3,4-c]pyridine

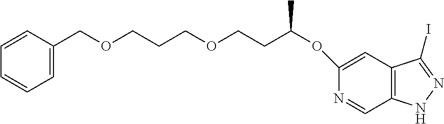 [SMILES: C[C@H](CCOCCCOCc1ccccc1)Oc1cc2c(I)n[nH]c2cn1]

To a solution of 5-[(1R)-3-(3-benzyloxypropoxy)-1-methyl-propoxy]-3-iodo-1H-pyrazolo[3,4-c]pyridine (1.82 g, 5.12 mmol) in acetone (70 mL) was added N-Iodosuccinimide (1.26 g, 5.63 mmol). The reaction mixture was stirred at RT for 18 h. The reaction mixture was quenched with saturated aqueous sodium bicarbonate solution and extracted with ethyl acetate. The combined organic layer was washed with brine, dried over anhydrous sodium sulfate, filtered and concentrated under reduced pressure to afford 5-[(1R)-3-(3-benzyloxypropoxy)-1-methyl-propoxy]-3-iodo-1H-pyrazolo[3,4-c]pyridine as an orange oil which was used in the next step without further purification.

LCMS method F: [M+H]⁺=482.0, t_{R}=3.07 min

### Preparation of Intermediate 661: 5-[(1R)-3-(3-benzyloxypropoxy)-1-methyl-propoxy]-3-iodo-1-tetrahydropyran-2-yl-pyrazolo[3,4-c]pyridine

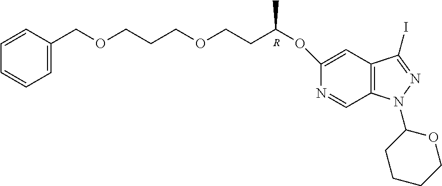

To a solution of 5-[(1R)-3-(3-benzyloxypropoxy)-1-methyl-propoxy]-3-iodo-1H-pyrazolo[3,4-c]pyridine (2.50 g, 5.12 mmol) in THF (70 mL) was added p-toluenesulfonic acid monohydrate (88 mg, 0.513 mmol) and 3,4-dihydro-2H-pyran (1.9 mL, 20.5 mmol). The reaction mixture was stirred at 60° C. for 96 h. Additional p-toluenesulfonic acid monohydrate (44 mg, 0.257 mmol) and 3,4-dihydro-2H-pyran (0.94 mL, 10.252 mmol) were added and the reaction was stirred at 60° C. for 18 h. Additional p-toluenesulfonic acid monohydrate (44 mg, 0.257 mmol) and 3,4-dihydro-2H-pyran (0.94 mL, 10.252 mmol) were added and the reaction was stirred at 60° C. for 18 h. The reaction mixture was diluted with saturated aqueous sodium bicarbonate solution and ethyl acetate. The phases were separated and the aqueous layer was extracted with ethyl acetate. The combined organic layer was washed with brine, dried over anhydrous sodium sulfate, filtered and evaporated under reduced pressure. The residue was purified on silica gel column chromatography using cyclohexane/ethyl acetate 100/0 to 80/20 as eluent to afford 5-[(1R)-3-(3-benzyloxypropoxy)-1-methyl-propoxy]-3-iodo-1-tetrahydropyran-2-yl-pyrazolo[3,4-c]pyridine as a yellow oil.

LCMS method F: [M+H]⁺=566.3, t_{R}=3.57 min

### Preparation of Intermediate 662: 2-[[4-[5-[(1R)-3-(3-benzyloxypropoxy)-1-methyl-propoxy]-1-tetrahydropyran-2-yl-pyrazolo[3,4-c]pyridin-3-yl]pyrazol-1-yl]methoxy] ethyl-trimethyl-silane

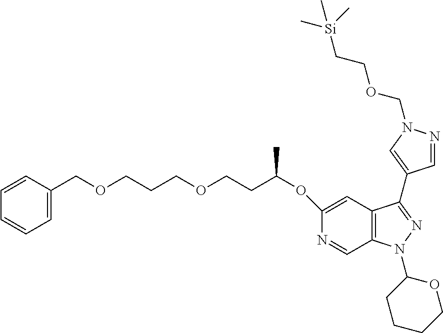 [SMILES: C[C@H](CCOCCCOCc1ccccc1)Oc1cc2c(-c3cnn(COCC[Si](C)(C)C)c3)nn(C3CCCCO3)c2cn1]

To a suspension of 5-[(1R)-3-(3-benzyloxypropoxy)-1-methyl-propoxy]-3-iodo-1-tetrahydro pyran-2-yl-pyrazolo[3,4-c]pyridine (2.01 g, 3.55 mmol) in dioxane (35 mL) and water (3.5 mL) was added trimethyl-[2-[[4-(4,4,5,5-tetramethyl-1,3,2-dioxaborolan-2-yl)pyrazol-1-yl] methoxy]ethyl]silane (1.5 g, 4.62 mmol), and potassium phosphate tribasic (2.26 g, 10.67 mmol). The reaction mixture was purged with argon for 15 min then tetrakis(triphenylphosphine)palladium(0) (206 mg, 0.178 mmol) and Xphos (170 mg, 0.356 mmol) were added. The reaction mixture was stirred at 100° C. for 18 h. The reaction mixture was evaporated under reduced pressure. Ethyl acetate and water were added. After separation, the aqueous layer was extracted with ethyl acetate and the combined organic layer was washed with brine, dried over sodium sulfate, filtered and evaporated under reduced pressure. The residue was purified by silica gel column chromatography using cyclohexane/ethyl acetate 100/0 to 60/40 as eluent to afford 2-[[4-[5-[(1R)-3-(3-benzyloxypropoxy)-1-methyl-propoxy]-1-tetrahydropyran-2-yl-pyrazolo[3,4-c]pyridin-3-yl]pyrazol-1-yl]methoxy]ethyl-trimethyl-silane as a yellow oil.

LCMS method F: [M+H]⁺=636.5, t_{R}=3.73 min

### Preparation of Intermediate 663: 3-[(3R)-3-[1-tetrahydropyran-2-yl-3-[1-(2-trimethylsilylethoxymethyl)pyrazol-4-yl]pyrazolo[3,4-c]pyridin-5-yl]oxybutoxy] propan-1-ol

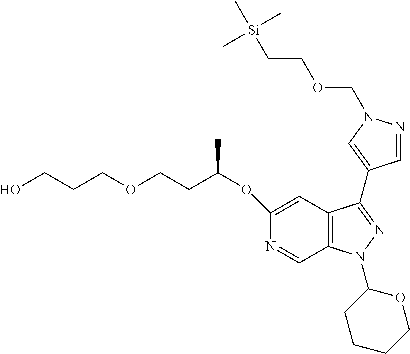 [SMILES: C[C@H](CCOCCCO)Oc1cc2c(-c3cnn(COCC[Si](C)(C)C)c3)nn(C3CCCCO3)c2cn1]

To a solution of 2-[[4-[5-[(1R)-3-(3-benzyloxypropoxy)-1-methyl-propoxy]-1-tetrahydro pyran-2-yl-pyrazolo[3,4-c]pyridin-3-yl]pyrazol-1-yl]methoxy]ethyl-trimethyl-silane (1.10 g, 1.73 mmol) in acetonitrile (240 mL) was added 2,3-dichloro-5,6-dicyano-p-benzoquinone (591 mg, 2.60 mmol). The reaction mixture was stirred at RT under photoirradiation (390 nm) for 4 h. The reaction mixture was quenched with saturated aqueous sodium bicarbonate solution and extracted with ethyl acetate. The organic layers were combined and washed with brine, dried over anhydrous sodium sulfate and evaporated under reduced pressure. The residue was purified by silica gel column chromatography using cyclohexane/ethyl acetate 100/0 to 30/70 as eluent to afford 3-[(3R)-3-[1-tetrahydropyran-2-yl-3-[1-(2-trimethylsilylethoxymethyl) pyrazol-4-yl]pyrazolo[3,4-c]pyridin-5-yl]oxybutoxy] propan-1-ol as an orange oil.

LCMS method F: [M+H]⁺=546.5, t_{R}=3.06 min

### Preparation of Intermediate 664: 3-[(3R)-3-[3-(1H-pyrazol-4-yl)-1-tetrahydropyran-2-yl-pyrazolo[3,4-c]pyridin-5-yl]oxybutoxy]propan-1-ol

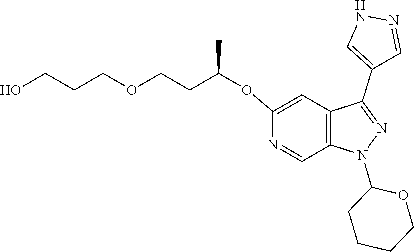 [SMILES: C[C@H](CCOCCCO)Oc1cc2c(-c3cn[nH]c3)nn(C3CCCCO3)c2cn1]

To a solution of (3R)-3-[(3R)-3-[1-tetrahydropyran-2-yl-3-[2-(2-trimethylsilyl ethoxymethyl) triazol-4-yl]indazol-5-yl]oxybutoxy]butan-1-ol (690 mg, 1.264 mmol) in TH (9 mL) at RT was added tetrabutyl ammonium fluoride (1M solution in THF) (2.52 mL, 2.52 mmol). The reaction mixture was stirred at 60° C. for 24 h. The reaction mixture was quenched with saturated ammonium chloride solution and extracted with ethyl acetate. The combined organic laver was dried over anhydrous sodium sulfate and concentrated under reduced pressure. The residue was purified by silica gel column chromatography using cyclohexane/ethyl acetate 100/0 to 0/100 as eluent to afford 3-[(3R)-3-[3-(1H-pyrazol-4-yl)-1-tetrahydropyran-2-yl-pyrazolo[3,4-c]pyridin-5-yl]oxybutoxy]propan-1-ol as a colorless oil.

LCMS method F: [M+H]⁺=416.3, t_{R}=2.06 min

### Preparation of Intermediate 665: (12R)-12-methyl-18-(oxan-2-yl)-9,13-dioxa-4,5, 15,18,19-pentaazatetracyclo[12.5.2.1^{2,5}.0^{17,20}]docosa-1(19),2(22),3,14(21),15,17(20)-hexaene

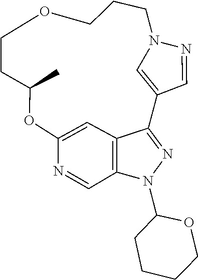 [SMILES: C[C@@H]1CCOCCCn2cc(cn2)-c2nn(C3CCCCO3)c3cnc(cc23)O1]

To a suspension of cesium carbonate (704 mg, 2.160 mmol) in anhydrous acetonitrile (492 mL) at 80° C. was added dropwise 3-[(3R)-3-[3-(1-methylsulfonylpyrazol-4-yl)-1-tetrahydropyran-2-yl-pyrazolo[3,4-c]pyridin-5-yl]oxybutoxy]propyl methanesulfonate (247 mg, 0.432 mmol) in anhydrous acetonitrile (492 mL). After addition the reaction mixture was stirred at 80° C. for 18 h. The reaction mixture was filtered and concentrated under reduced pressure. The residue was diluted with ethyl acetate and water. The phases were separated and the aqueous phase was extracted with ethyl acetate. The combined organic layer was washed with brine, dried over anhydrous sodium sulfate, filtered and evaporated under reduced pressure. The residue was purified by silica gel column chromatography using cyclohexane/(ethyl acetate/ethanol 3/1) 100/0 to 90/10 as eluent to afford (12R)-12-methyl-18-(oxan-2-yl)-9,13-dioxa-4,5,15,18,19-pentaazatetracyclo[12.5.2.1^{2,5}.0^{17,20}]docosa-1(19),2(22),3,14(21),15,17(20)-hexaene as an opaque solid.

LCMS method F: [M+H]⁺=398.3, t_{R}=2.33 min

### Preparation of Example 245: (12R)-12-methyl-9,13-dioxa-4,5,15,18,19-pentaaza tetracyclo[12.5.2.1^{2,5}.0^{17,20}]docosa-1(19),2(22),3,14(21),15,17(20)-hexaene

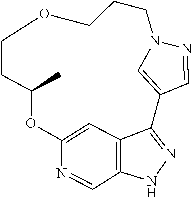 [SMILES: C[C@@H]1CCOCCCn2cc(cn2)-c2n[nH]c3cnc(cc23)O1]

To a solution of (12R)-12-methyl-18-(oxan-2-yl)-9,13-dioxa-4,5,15,18,19-pentaazatetracyclo [12.5.2.1^{2,5}.0^{17,20}]docosa-1(19),2(22),3,14(21),15,17(20)-hexaene (102 mg, 0.257 mmol) in methanol (5.6 mL) and water (0.8 mL) was added p-toluenesulfonic acid monohydrate (244 mg, 1.285 mmol). The reaction mixture was stirred at 65° C. for 18 h. The reaction mixture was diluted with ethyl acetate and saturated aqueous sodium hydrogen carbonate solution. After separation, the aqueous layer was extracted with ethyl acetate. The combined organic layer was washed brine, dried over anhydrous sodium sulfate, filtered and evaporated under reduced pressure. The residue was triturated with acetonitrile. The resulting solid was filtered and dried to afford (12R)-12-methyl-9,13-dioxa-4,5,15,18,19-pentaazatetracyclo[12.5.2.1^{2,5}.0^{17,20}] docosa-1(19),2(22),3,14(21),15,17(20)-hexaene as a white solid.

LCMS method F: [M+H]⁺=314.3, t_{R}=1.71 min

LCMS method G: [M+H]⁺=314.3, t_{R}=1.76 min

¹H NMR (400 MHz, DMSO) 13.14 (1H, bs), 8.69 (1H, s), 8.63 (1H, d, J=0.85 Hz), 7.71 (1H, d, J=0.58 Hz), 7.35 (1H, s), 4.57-4.40 (2H, m), 4.37-4.27 (1H, m), 3.78-3.70 (1H, m), 3.70-3.61 (2H, m), 3.58-3.49 (1H, m), 2.43-2.31 (1H, m), 2.28-2.05 (2H, m), 1.62-1.52 (1H, m), 1.44 (3H, d, J=6.07 Hz) ppm.

### Example 246: 12S)-12-methyl-8,11,14-trioxa-4,5,16,19,20-pentaazatetracyclo[13.5.2.1^{2,5}. 0^{18,21}]tricosa-1(20),2(23),3,15(22),16,18(21)-hexaene

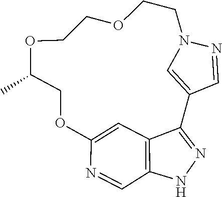 [SMILES: C[C@H]1COc2cc3c(n[nH]c3cn2)-c2cnn(c2)CCOCCO1]

Example 246 is prepared according to the synthesis route described below.

### Preparation of Intermediate 666: [[(2S)-2-[2-(2-benzyloxyethoxy)ethoxy]propoxy]-diphenyl-methyl]benzene

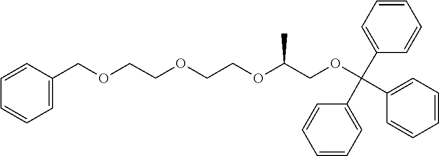 [SMILES: C[C@@H](COC(c1ccccc1)(c1ccccc1)c1ccccc1)OCCOCCOCc1ccccc1]

To a suspension of sodium hydride (60% dispersion in mineral oil) (2.47 g, 61.8 mmol) in DMF (30 mL) at 0° C. was added dropwise a solution of (2S)-1-trityloxypropan-2-ol (13.11 g, 41.2 mmol) in DMF (50 mL). The reaction mixture was stirred at RT for 15 min then cooled to 0° C. and a solution of 2-(2-benzyloxyethoxy)ethyl methanesulfonate intermediate 145 (7.53 g, 27.46 mmol) in DMF (30 mL) was added. The resulting solution was stirred at 0° C. for 5 min then brought up to RT and stirred for 15 h. The reaction mixture was quenched with saturated aqueous NH₄Cl solution. Ethyl acetate was added and phases were separated. The aqueous layer was extracted with ethyl acetate. The combined organic extracts were washed with brine, dried over anhydrous sodium sulfate, filtered and concentrated under reduced pressure. The residue was purified by silica gel column chromatography using cyclohexane/ethyl acetate 100/0 to 70/30 as eluent to afford [[(2S)-2-[2-(2-benzyloxyethoxy)ethoxy]propoxy]-diphenyl-methyl]benzene as a yellow oil.

¹H NMR (400 MHz, CDCl₃) 7.36-7.20 (20H, m), 4.57 (2H, s), 3.77-3.59 (9H, m), 3.25-3.18 (1H, m), 3.04-2.97 (1H, m), 1.18 (3H, d, J=6.39 Hz) ppm.

### Preparation of Intermediate 667: (2S)-2-[2-(2-benzyloxyethoxy)ethoxy]propan-1-ol

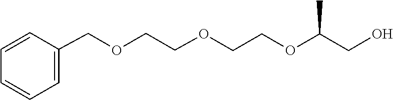 [SMILES: C[C@@H](CO)OCCOCCOCc1ccccc1]

To a solution of [[(2S)-2-[2-(2-benzyloxyethoxy)ethoxy]propoxy]-diphenyl-methyl]benzene (17.36 g, 27.46 mmol) in methanol (150 mL) was added p-toluenesulfonic acid monohydrate (665 mg, 3.50 mmol). The reaction mixture was stirred at RT for 15 h. The solvent was evaporated under reduced pressure and the residue was partitioned between ethyl acetate and saturated aqueous sodium bicarbonate solution. The aqueous layer was extracted with ethyl acetate and the combined organic extracts were washed with brine, dried over anhydrous sodium sulfate, filtered and concentrated under reduced pressure. The residue was purified by silica gel column chromatography using cyclohexane/ethyl acetate 100/0 to 50/50 as eluent to afford (2S)-2-[2-(2-benzyloxyethoxy)ethoxy]propan-1-ol as a colorless oil.

LCMS method F: [M+H]⁺=255.1, t_{R}=2.00 min

### Preparation of Intermediate 668: 2-[(2S)-2-[2-(2-benzyloxyethoxy)ethoxy]propoxy]-4-methyl-5-nitro-pyridine

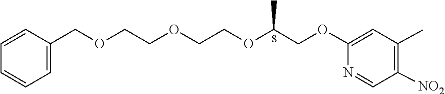

To a solution of 2-chloro-4-methyl-5-nitro-pyridine (1.00 g, 5.75 mmol) and ((2S)-2-[2-(2-benzyloxyethoxy) ethoxy]propan-1-ol (1.21 g, 4.79 mmol) in dry toluene (50 mL) was added cesium carbonate (2.19 g, 6.71 mmol), Pd₂(dba)₃ (58 mg, 0.10 mmol) and rac-BINAP (180 mg, 0.29 mmol). The reaction mixture was purged with argon then stirred at 110° C. for 16 h. Additional Pd₂(dba)₃ (29 mg, 0.050 mmol), rac-BINAP (90 mg, 0.145 mmol), cesium carbonate (1.095 g, 3.33 mmol) were added and the reaction mixture was stirred at 110° C. for 18 h. The reaction mixture was washed with water and ethyl acetate. The phases were separated and the aqueous layer was extracted with ethyl acetate. The organic layer was washed with brine, dried over anhydrous sodium sulfate, filtered and evaporated under reduced pressure. The residue was purified by silica gel column chromatography using cyclohexane/ethyl acetate 100/0 to 80/20 as eluent to afford 2-[(2S)-2-[2-(2-benzyloxyethoxy)ethoxy]propoxy]-4-methyl-5-nitro-pyridine as an orange oil.

LCMS method F: [M+H]⁺=391.1, t_{R}=2.97 min

### Preparation of Intermediate 669: 6-[(2S)-2-[2-(2-benzyloxyethoxy)ethoxy]propoxy]-4-methyl-pyridin-3-amine

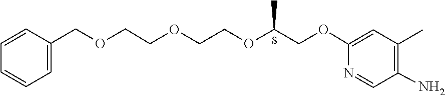

To a solution of 2-[(2S)-2-[2-(2-benzyloxyethoxy)ethoxy]propoxy]-4-methyl-5-nitro-pyridine (2.30 g, 5.91 mmol) in ethanol (48 mL) and water (12 mL) was added ammonium chloride (3.13 g, 59.11 mmol) and iron (3.31 g, 59.11 mmol) under argon atmosphere. The reaction mixture was stirred at 70° C. for 4 h then at 50° C. for 18 h. Additional iron (1.65 g, 29.56 mmol) and ammonium chloride (1.56 g, 29.56 mmol) were added and the reaction mixture was stirred at 70° C. for 6 h. The reaction mixture was filtered over a Celite pad and evaporated under reduced pressure. The oily residue was portioned between ethyl acetate and saturated aqueous sodium bicarbonate solution. The phases were separated and the aqueous layer was extracted with ethyl acetate. The organic phase was dried over anhydrous sodium sulfate, filtered and evaporated under reduced pressure to afford 6-[(2S)-2-[2-(2-benzyloxyethoxy)ethoxy]propoxy]-4-methyl-pyridin-3-amine as an orange oil which was used in the next step without further purification.

LCMS method F: [M+H]⁺=361.3, t_{R}=1.85 min

### Preparation of Intermediate 670: N-[6-[(2S)-2-[2-(2-benzyloxyethoxy) ethoxy]propoxy]-4-methyl-3-pyridyl]acetamide

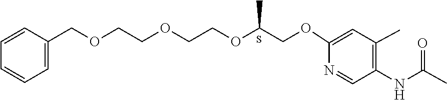

To a solution of 6-[(2S)-2-[2-(2-benzyloxyethoxy)ethoxy]propoxy]-4-methyl-pyridin-3-amine (2.06 g, 5.73 mmol) and triethylamine (2.38 mL, 17.21 mmol) in dichloromethane (20 mL) at 0° C. was added anhydride acetic (975 μL, 10.32 mmol). The reaction mixture was stirred at RT for 2 h. Saturated aqueous sodium bicarbonate solution and dichloromethane were added and the phases were separated. The aqueous layer was extracted with dichloromethane. The combined organic layers were washed with brine, dried over anhydrous sodium sulfate, filtered and evaporated under reduced pressure. The residue was filtered over silica gel pad using ethyl acetate as eluent to afford N-[6-[(2S)-2-[2-(2-benzyloxyethoxy)ethoxy]propoxy]-4-methyl-3-pyridyl]acetamide as an orange oil.

LCMS method F: [M+H]⁺=403.2, t_{R}=2.35 min

### Preparation of Intermediate 671: Mixture of 1-[5-[(2S)-2-[2-(2-benzyloxyethoxy) ethoxy]propoxy]pyrazolo[3,4-c]pyridin-1-yl]ethenone and 1-[5-[(2S)-2-[2-(2-benzyloxyethoxy)ethoxy]propoxy]pyrazolo[3,4-c]pyridin-2-yl]ethanone

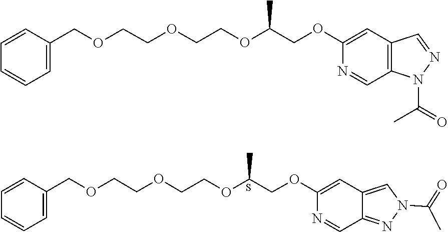

To a solution of N-[6-[(2S)-2-[2-(2-benzyloxyethoxy)ethoxy]propoxy]-4-methyl-3-pyridyl] acetamide (2.15 g, 5.34 mmol), potassium acetate (786 mg, 8.016 mmol) and anhydride acetic (1.01 mL, 10.68 mmol) in dry toluene (40 mL) at 80° C. was added dropwise tert-butyl nitrite (3.80 mL, 32.06 mmol). The reaction mixture was stirred at 80° C. for 18 h. Ethyl acetate and saturated aqueous sodium bicarbonate solution were added. The phases were separated and the aqueous layer was extracted with ethyl acetate. The combined organic layer was washed with brine, dried over anhydrous sodium sulfate, filtered and evaporated under reduced pressure to afford a mixture of 1-[5-[(2S)-2-[2-(2-benzyloxyethoxy)ethoxy]propoxy]pyrazolo[3,4-c]pyridin-1-yl] ethanone and 1-[5-[(2S)-2-[2-(2-benzyloxyethoxy)ethoxy]propoxy]pyrazolo [3,4-c]pyridin-2-yl]ethanone as an orange oil.

LCMS method F: [M+H]⁺=414.1, t_{R}=2.74 min and [M+H]⁺=414.1, t_{R}=2.86 min

### Preparation of Intermediate 672: 5-[(2S)-2-[2-(2-benzyloxyethoxy)ethoxy]propoxy]-1H-pyrazolo[3,4-c]pyridine

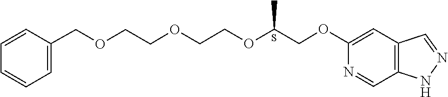

To a mixture of 1-[5-[(2S)-2-[2-(2-benzyloxyethoxy)ethoxy]propoxy]pyrazolo[3,4-c]pyridin-1-yl]ethenone and 1-[5-[(2S)-2-[2-(2-benzyloxyethoxy)ethoxy]propoxy]pyrazolo[3,4-c] pyridin-2-yl]ethanone (1.95 g, 4.72 mmol) in methanol (35 ml) was added ammonia solution (4M in methanol) (8.3 mL, 33.06 mmol). The reaction mixture was stirred at RT for 2 h. The reaction mixture was evaporated to dryness and the residue was purified by silica gel column chromatography using cyclohexane/ethyl acetate 100/0 to 40/60 as eluent to afford 5-[(2S)-2-[2-(2-benzyloxyethoxy)ethoxy]propoxy]-1H-pyrazolo[3,4-c]pyridine as an orange oil.

LCMS method F: [M+H]⁺=372.2, t_{R}=2.28 min

### Preparation of Intermediate 673: 5-[(2S)-2-[2-(2-benzyloxyethoxy)ethoxy]propoxy]-3-iodo-1H-pyrazolo[3,4-c]pyridine

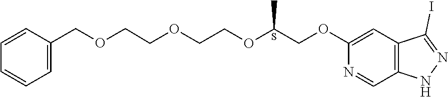

To a solution of 5-[(2S)-2-[2-(2-benzyloxyethoxy)ethoxy]propoxy]-1H-pyrazolo[3,4-c]pyridine (1.36 g, 3.37 mmol) in acetone (50 mL) was added portionwise N-iodosuccinimide (910 mg, 4.04 mmol). The reaction mixture was stirred at RT for 18 h. The reaction mixture was quenched with saturated aqueous sodium bicarbonate solution. The phases were separated and the aqueous layer was extracted with ethyl acetate. The combined organic layer was washed with brine, dried over anhydrous sodium sulfate, filtered and concentrated under reduced pressure to afford 5-[(2S)-2-[2-(2-benzyloxyethoxy)ethoxy]propoxy]-3-iodo-1H-pyrazolo[3,4-c]pyridine as a an orange oil.

LCMS method F: [M+H]⁺=498.1, t_{R}=2.78 min

### Preparation of Intermediate 674: 5-[(2S)-2-[2-(2-benzyloxyethoxy)ethoxy]propoxy]-3-iodo-1-tetrahydropyran-2-yl-pyrazolo[3,4-c]pyridine

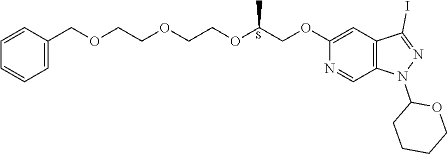

To a solution of 5-[(2S)-2-[2-(2-benzyloxyethoxy)ethoxy]propoxy]-3-iodo-1H-pyrazolo[3,4-c]pyridine (1.74 g, 3.50 mmol) in THF (50 mL) was added p-toluenesulfonic acid monohydrate (60 mg, 0.35 mmol) and 3,4-dihydro-2H-pyran (1.3 mL, 14.0 mmol). The reaction mixture was stirred at 60° C. for 72 h. Additional p-toluenesulfonic acid monohydrate (30 mg, 0.175 mmol) and 3,4-dihydro-2H-pyran (0.64 mL, 7.0 mmol) were added and the reaction mixture was stirred at 60° C. for 5 h. The reaction mixture was diluted with saturated aqueous sodium bicarbonate solution and ethyl acetate. The phases were separated. The aqueous layer was extracted with ethyl acetate and the combined organic layer was washed with brine, dried over anhydrous sodium sulfate, filtered and evaporated under reduced pressure. The residue was purified by silica gel column chromatography using cyclohexane/ethyl acetate 100/0 to 70/30 as eluent to afford 5-[(2S)-2-[2-(2-benzyloxyethoxy)ethoxy]propoxy]-3-iodo-1-tetrahydropyran-2-yl-pyrazolo[3,4-c]pyridine as a pale yellow oil.

LCMS method F: [M+H]⁺=582.2, t_{R}=3.24 min

### Preparation of Intermediate 675: 2-[[4-[5-[(2S)-2-[2-(2-benzyloxyethoxy) ethoxy] propoxy]-1-tetrahydropyran-2-yl-pyrazolo[3,4-c]pyridin-3-yl]pyrazol-1-yl]methoxy] ethyl-trimethyl-silane

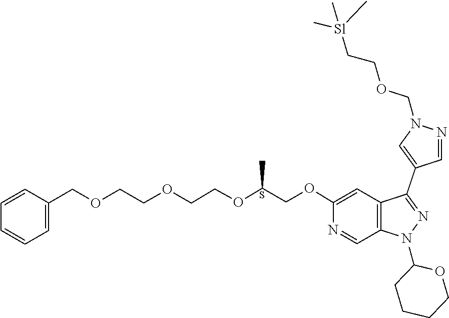

To a suspension of 5-[(2S)-2-[2-(2-benzyloxyethoxy)ethoxy]propoxy]-3-iodo-1-tetrahydro pyran-2-yl-pyrazolo[3,4-c]pyridine (1.51 g, 2.61 mmol) in dioxane (25 mL) and water (2.5 mL) was added trimethyl-[2-[[4-(4,4,5,5-tetramethyl-1,3,2-dioxaborolan-2-yl)pyrazol-1-yl]methoxy]ethyl]silane (1.10 g, 3.39 mmol) and potassium phosphate tribasic (1.66 g, 7.83 mmol). The reaction mixture was purged with argon for 15 min then tetrakis(triphenylphosphine)palladium(0) (151 mg, 0.131 mmol) and Xphos (124 mg, 0.261 mmol) were added. The reaction mixture was stirred at 100° C. for 18 h. The reaction mixture was evaporated to dryness. Ethyl acetate and water were added. After separation, the aqueous layer was extracted with ethyl acetate. The combined organic layer was washed with brine, dried over anhydrous sodium sulfate, filtered and evaporated under reduced pressure. The residue was purified by silica gel column chromatography using cyclohexane/ethyl acetate 100/0 to 60/40 as eluent to afford 2-[[4-[5-[(2S)-2-[2-(2-benzyloxyethoxy)ethoxy]propoxy]-1-tetrahydropyran-2-yl-pyrazolo[3,4-c]pyridin-3-yl]pyrazol-1-yl]methoxy]ethyl-trimethyl-silane as a yellow oil.

LCMS method F: [M+H]⁺=652.6, t_{R}=3.51 min

### Preparation of Intermediate 676: 2-[2-[(1S)-1-methyl-2-[1-tetrahydropyran-2-yl-3-[1-(2-trimethylsilylethoxymethyl)pyrazol-4-yl]pyrazolo[3,4-c]pyridin-5-yl]oxy-ethoxy]ethoxy]ethanol

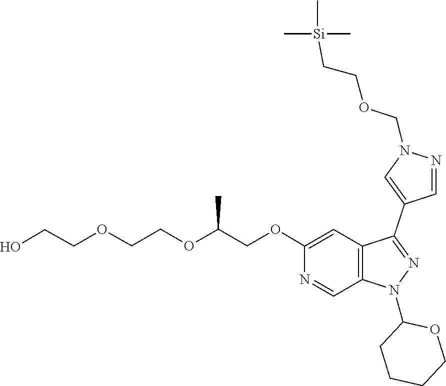 [SMILES: C[C@@H](COc1cc2c(-c3cnn(COCC[Si](C)(C)C)c3)nn(C3CCCCO3)c2cn1)OCCOCCO]

To a solution of 2-[[4-[5-[(2S)-2-[2-(2-benzyloxyethoxy)ethoxy]propoxy]-1-tetrahydropyran-2-yl-pyrazolo[3,4-c]pyridin-3-yl]pyrazol-1-yl]methoxy]ethyl-trimethyl-silane (132 mg, 0.202 mmol) in acetonitrile (40 mL) was added 2,3-dichloro-5,6-dicyano-p-benzoquinone (69 mg, 0.303 mmol). The reaction mixture was stirred at RT under photoirradiation (390 nm) for 4 h. The reaction mixture was quenched with saturated aqueous sodium bicarbonate solution and extracted with ethyl acetate. The organic layers were combined and washed with brine, dried over anhydrous sodium sulfate, filtered and evaporated under reduced pressure. The residue was purified by silica gel column chromatography using cyclohexane/ethyl acetate 100/0 to 20/80 as eluent to afford 2-[2-[(1S)-1-methyl-2-[1-tetrahydropyran-2-yl-3-[1-(2-trimethylsilylethoxymethyl)pyrazol-4-yl]pyrazolo[3,4-c]pyridin-5-yl]oxy-ethoxy]ethoxy] ethanol as a red oil.

LCMS method F: [M+H]⁺=562.4, t_{R}=2.94 min

### Preparation of Intermediate 677: 2-[2-[(1S)-1-methyl-2-[3-(1H-pyrazol-4-yl)-1-tetrahydropyran-2-yl-pyrazolo[3,4-c]pyridin-5-yl]oxy-ethoxy]ethoxy]ethanol

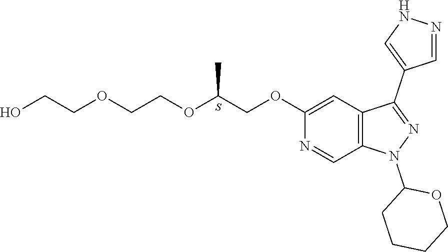

To a solution of 2-[2-[(1S)-1-methyl-2-[1-tetrahydropyran-2-yl-3-[1-(2-trimethylsilylethoxy methyl)pyrazol-4-yl]pyrazolo[3,4-c]pyridin-5-yl]oxy-ethoxy]ethoxy] ethanol (148 mg, 0.263 mmol) in THF (3 mL) at RT was added tetrabutylammonium fluoride (1M solution in THF) (527 μL, 0.527 mmol). The reaction mixture was stirred at 60° C. for 18 h. Additional tetrabutylammonium fluoride (1M solution in THF) (527 μL, 0.527 mmol) was added and the reaction mixture was stirred at 60° C. for 18 h. The reaction mixture was quenched with saturated ammonium chloride solution and extracted with ethyl acetate. The combined organic layer was dried over anhydrous sodium sulfate and concentrated under reduced pressure. The residue was purified by silica gel column chromatography using cyclohexane/(ethyl acetate/ethanol 3/1) 100/0 to 90/10 as eluent to afford 2-[2-[(1S)-1-methyl-2-[3-(1H-pyrazol-4-yl)-1-tetrahydropyran-2-yl-pyrazolo[3,4-c]pyridin-5-yl]oxy-ethoxy] ethoxy]ethanol as an orange oil.

LCMS method F: [M+H]⁺=432.3, t_{R}=1.99 min

### Preparation of Intermediate 678: 2-[2-[(1S)-1-methyl-2-[3-(1-methylsulfonylpyrazol-4-yl)-1-tetrahydropyran-2-yl-pyrazolo[3,4-c]pyridin-5-yl]oxy-ethoxy]ethoxy]ethyl methanesulfonate

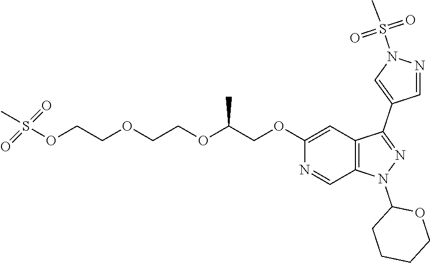 [SMILES: C[C@@H](COc1cc2c(-c3cnn(S(C)(=O)=O)c3)nn(C3CCCCO3)c2cn1)OCCOCCOS(C)(=O)=O]

To a solution of 2-[2-[(1S)-1-methyl-2-[3-(1H-pyrazol-4-yl)-1-tetrahydropyran-2-yl-pyrazolo[3,4-c]pyridin-5-yl]oxy-ethoxy]ethoxy]ethanol (49 mg, 0.113 mmol) and triethylamine (63 μL, 0.451 mmol) in dichloromethane (2 mL) at 0° C. was added methanesulfonyl chloride (26 μL, 0.338 mmol). The reaction mixture was stirred at RT for 18 h. The reaction mixture was diluted with water and extracted with dichloromethane. The combined organic layer was washed with brine, dried over anhydrous sodium sulfate and concentrated under reduced pressure to afford 2-[2-[(1S)-1-methyl-2-[3-(1-methylsulfonylpyrazol-4-yl)-1-tetrahydropyran-2-yl-pyrazolo[3,4-c]pyridin-5-yl]oxy-ethoxy] ethoxy]ethyl methanesulfonate as a yellow oil, which was used in the next step without further purification.

LCMS method F: [M+H]⁺=588.2, t_{R}=2.57 min

### Preparation of Intermediate 679: (12S)-12-methyl-19-(oxan-2-yl)-8,11,14-trioxa-4,5, 16,19,20-pentaazatetracyclo[13.5.2.1^{2,5}.0^{18,21}]tricosa-1(20),2(23),3,15(22),16,18(21)-hexane

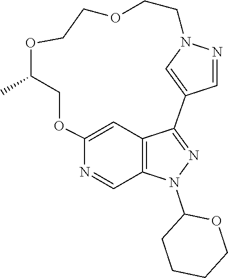 [SMILES: C[C@H]1COc2cc3c(nn(C4CCCCO4)c3cn2)-c2cnn(c2)CCOCCO1]

To a suspension of cesium carbonate (150 mg, 0.46 mmol) in anhydrous acetonitrile (50 mL) at 80° C. was added dropwise 2-[2-[(1S)-1-methyl-2-[3-(1-methylsulfonylpyrazol-4-yl)-1-tetrahydropyran-2-yl-pyrazolo[3,4-c]pyridin-5-yl]oxy-ethoxy]ethoxy]ethyl methanesulfonate (54 mg, 0.092 mmol) in anhydrous acetonitrile (50 mL). The reaction mixture was stirred at 80° C. for 18 h. The reaction mixture was filtered and concentrated under reduced pressure. The residue was diluted with ethyl acetate and water. after separation, the aqueous phase was extracted with ethyl acetate. The combined organic layer was washed with brine, dried over anhydrous sodium sulfate, filtered and evaporated under reduced pressure. The residue was purified by silica gel column chromatography using cyclohexane/(ethyl acetate/ethanol 3/1) 100/0 to 90/10 as eluent to afford (12S)-12-methyl-19-(oxan-2-yl)-8,11,14-trioxa-4,5,16,19,20-pentaazatetracyclo[13.5.2.1^{2,5}.0^{18,21}]tricosa-1(20),2(23),3,15(22),16,18(21)-hexaene as an opaque solid.

LCMS method F: [M+H]⁺=414.4, t_{R}=2.08 min Preparation of Example 246: (12S)-12-methyl-8,11,14-trioxa-4,5,16,19,20-pentaazatetracyclo[13.5.2.1^{2,5}.0^{18,21}]tricosa-1(20),2(23),3,15(22),16,18(21)-hexaene

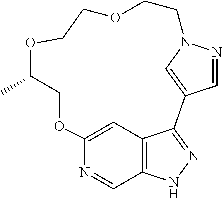 [SMILES: C[C@H]1COc2cc3c(n[nH]c3cn2)-c2cnn(c2)CCOCCO1]

To a solution of (12S)-12-methyl-19-(oxan-2-yl)-8,11,14-trioxa-4,5,16,19,20-pentaazatetra cyclo[13.5.2.1^{2,5}.0^{18,21}]tricosa-1(20),2(23),3,15(22),16,18(21)-hexaene (22 mg, 0.053 mmol) in methanol (1.5 mL) and water (0.2 mL) was added p-toluenesulfonic acid monohydrate (51 mg, 0.266 mmol). The reaction mixture was stirred at 65° C. for 18 h. The reaction mixture was diluted with ethyl acetate and a saturated aqueous sodium hydrogen carbonate solution. After separation, the aqueous layer was extracted with ethyl acetate. The combined organic layers were washed brine, dried over anhydrous sodium sulfate, filtered and evaporated under reduced pressure. The residue was triturated with acetonitrile and the resulting solid was filtered and dried to afford (12S)-12-methyl-8,11,14-trioxa-4,5,16,19,20-pentaazatetracyclo[13.5.2.1^{2,5}.0^{18,21}]tricosa-1(20),2(23),3,15(22),16,18(21)-hexaene as a white solid.

LCMS method F: [M+H]⁺=330.3, t_{R}=1.55 min

LCMS method G: [M+H]⁺=330.3, t_{R}=1.61 min

¹H NMR (400 MHz, DMSO) 13.22 (1H, bs), 8.62 (1H, s), 8.50 (1H, s), 7.83 (1H, s), 7.77 (1H, s), 4.33-4.25 (4H, m), 3.90-3.78 (3H, m), 3.77-3.57 (4H, m), 1.22 (3H, d, J=6.45 Hz) ppm.

Table 1 provides the example number, the IUPAC name and the general scheme according to which the compounds have been made.

TABLE 1

| Example | | General |
|---|---|---|
| number | IUPAC Name | scheme |
| Example 1 | 4-(morpholin-4-yl)-7,10-dioxa-13,18,19- | Scheme A |
| | triazatetracyclo[12.5.2.1^{2,6}.0^{17,20}]docosa- | |
| | 1(19),2(22),3,5,14(21),15,17(20)-heptaene | |
| Example 2 | 7,10,14-trioxa-4,19,20- | Scheme A |
| | triazatetracyclo[13.5.2.1^{2,6}.0^{18,21}]tricosa- | |
| | 1(20),2(23),3,5,15(22),16,18(21)-heptaene | |
| Example 3 | 4-(morpholin-4-yl)-7,11,14-trioxa-19,20- | Scheme A |
| | diazatetracyclo[13.5.2.1^{2,6}.0^{18,21}]tricosa- | |
| | 1(20),2(23),3,5,15(22),16,18(21)-heptaene | |
| Example 4 | 7,10-dioxa-4,14,19,20- | Scheme A |
| | tetraazatetracyclo[13.5.2.1^{2,6}.0^{18,21}]tricosa- | |
| | 1(20),2(23),3,5,15(22),16,18(21)-heptaene | |
| Example 5 | 9,13-dioxa-4,5,18,19- | Scheme B |
| | tetraazatetracyclo[12.5.2.1^{2,5}.0^{17,20}]docosa- | |
| | 1(19),2(22),3,14(21),15,17(20)-hexaene | |
| Example 6 | 10,14-dioxa-4,5,19,20- | Scheme B |
| | tetraazatetracyclo[13.5.2.1^{2,5}.0^{18,21}]tricosa- | |
| | 1(20),2(23),3,15(22),16,18(21)-hexaene | |
| Example 7 | (13R)-13-methyl-7,11,14-trioxa-5,19,20,23- | Scheme C |
| | tetraazatetracyclo[13.5.2.1^{2,6}.0^{18,21}]tricosa- | |
| | 1(20),2(23),3,5,15(22),16,18(21)-heptaene | |
| Example 8 | (6R)-6-methyl-9,13-dioxa-4,5,18,19- | Scheme B |
| | tetraazatetracyclo[12.5.2.1^{2,5}.0^{17,20}]docosa- | |
| | 1(19),2(22),3,14(21),15,17(20)-hexaene | |
| Example 9 | (7S,13S)-7,13-dimethyl-8,11,14-trioxa-4,19,20- | Scheme D |
| | triazatetracyclo[13.5.2.1^{2,6}.0^{18,21}]tricosa- | |
| | 1(20),2(23),3,5,15(22),16,18(21)-heptaene | |
| Example 10 | (7R,13S)-7,13-dimethyl-8,11,14-trioxa-4,19,20- | Scheme D |
| | triazatetracyclo[13.5.2.1^{2,6}.0^{18,21}]tricosa- | |
| | 1(20),2(23),3,5,15(22),16,18(21)-heptaene | |
| Example 11 | (8S,13R)-8,13-dimethyl-7,11,14-trioxa-4,19,20- | Scheme D |
| | triazatetracyclo[13.5.2.1^{2,6}.0^{18,21}]tricosa- | |
| | 1(20),2(23),3,5,15(22),16,18(21)-heptaene | |
| Example 12 | (8S,13R)-8,13-dimethyl-7,11,14-trioxa-4,19,20- | Scheme D |
| | triazatetracyclo[13.5.2.1^{2,6}.0^{18,21}]tricosa- | |
| | 1(20),2(23),3,5,15(22),16,18(21)-heptaene | |
| Example 13 | (7R,13R)-7,13-dimethyl-8,11,14-trioxa-4,19,20- | Scheme D |
| | triazatetracyclo[13.5.2.1^{2,6}.0^{18,21}]tricosa- | |
| | 1(20),2(23),3,5,15(22),16,18(21)-heptaene | |
| | hydrochloride | |
| Example 14 | (7S,13R)-7,13-dimethyl-8,11,14-trioxa-4,19,20- | Scheme D |
| | triazatetracyclo[13.5.2.1^{2,6}.0^{18,21}]tricosa- | |
| | 1(20),2(23),3,5,15(22),16,18(21)-heptaene | |
| | hydrochloride | |
| Example 15 | (6S)-6-methyl-9,13-dioxa-4,5,18,19- | Scheme B |
| | tetraazatetracyclo[12.5.2.1^{2,5}.0^{17,20}]docosa- | |
| | 1(19),2(22),3,14(21),15,17(20)-hexaene | |
| Example 16 | (13S)-13-methyl-7,10,14-trioxa-5,19,20,23- | Scheme C |
| | tetraazatetracyclo[13.5.2.1^{2,6}.0^{18,21}]tricosa- | |
| | 1(20),2(23),3,5,15(22),16,18(21)-heptaene | |
| Example 17 | (8R,13R)-8,13-dimethyl-7,11,14-trioxa-4,19,20- | Scheme D |
| | triazatetracyclo[13.5.2.1^{2,6}.0^{18,21}]tricosa- | |
| | 1(20),2(23),3,5,15(22),16,18(21)-heptaene | |
| Example 18 | (8R,13S)-8,13-dimethyl-7,11,14-trioxa-4,19,20- | Scheme D |
| | triazatetracyclo[13.5.2.1^{2,6}.0^{18,21}]tricosa- | |
| | 1(20),2(23),3,5,15(22),16,18(21)-heptaene | |
| Example 19 | (13S)-13-methyl-4-(pyrrolidin-1-yl)-7,11,14-trioxa- | Scheme E |
| | 5,19,20,23-tetraazatetracyclo[13.5.2.1^{2,6}.0^{18,21}]tricosa- | |
| | 1(20),2(23),3,5,15(22),16,18(21)heptaene | |
| Example 20 | (8S,13S)-8,13-dimethyl-7,10,14-trioxa-4,19,20- | Scheme D |
| | triazatetracyclo[13.5.2.1^{2,6}.0^{18,21}]tricosa- | |
| | 1(20),2(23),3,5,15(22),16,18(21)-heptaene | |
| Example 21 | (8R,13S)-8,13-dimethyl-7,10,14-trioxa-4,19,20- | Scheme D |
| | triazatetracyclo[13.5.2.1^{2,6}.0^{18,21}]tricosa- | |
| | 1(20),2(23),3,5,15(22),16,18(21)-heptaene | |
| Example 22 | (8S,13R)-8,13-dimethyl-7,10,14-trioxa-4,19,20- | Scheme D |
| | triazatetracyclo[13.5.2.1^{2,6}.0^{18,21}]tricosa- | |
| | 1(20),2(23),3,5,15(22),16,18(21)-heptaene | |
| Example 23 | (8R,13R)-8,13-dimethyl-7,10,14-trioxa-4,19,20- | Scheme D |
| | triazatetracyclo[13.5.2.1^{2,6}.0^{18,21}]tricosa- | |
| | 1(20),2(23),3,5,15(22),16,18(21)-heptaene | |
| Example 24 | (13S)-13-methyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 25 | (6R,12S)-6,12-dimethyl-9,13-dioxa-4,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 26 | (13S)-13-methyl-7,11,14-trioxa-5,19,20,23- | Scheme C |
| | tetraazatetracyclo[13.5.2.1^{2,6}.0^{18,21}]tricosa- | |
| | 1(20),2(23),3,5,15(22),16,18(21)-heptaene | |
| Example 27 | (13S)-4-(3-methoxyazetidin-1-yl)-13-methyl-7,11,14- | Scheme E |
| | trioxa-5,19,20,23- | |
| | tetraazatetracyclo[13.5.2.1^{2,6}.0^{18,21}]tricosa- | |
| | 1(20),2(23),3,5,15(22),16,18(21)heptaene | |
| Example 28 | (13S)-13-methyl-7,10,14-trioxa-19,20,23- | Scheme D |
| | triazatetracyclo[13.5.2.1^{2,6}.0^{18,21}]tricosa- | |
| | 1(20),2(23),3,5,15(22),16,18(21)-heptaene | |
| Example 29 | (13S)-4,13-dimethyl-7,11,14-trioxa-5,19,20,23- | Scheme A |
| | tetraazatetracyclo[13.5.2.1^{2,6}.0^{18,21}]tricosa- | |
| | 1(20),2(23),3,5,15(22),16,18(21)-heptaene | |
| Example 30 | (13S)-13-methyl-7,10-dioxa-5,14,19,20,23- | Scheme E |
| | pentaazatetracyclo[13.5.2.1^{2,6}.0^{18,21}]tricosa- | |
| | 1(20),2(23),3,5,15(22),16,18(21)-heptaene | |
| Example 31 | 7,7-dimethyl-9,13-dioxa-4,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 32 | (12S)-12-methyl-9,13-dioxa-4,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 33 | (13S)-13-methyl-7,10,14-trioxa-19,20,23- | Scheme D |
| | triazatetracyclo[13.5.2.1^{2,6}.0^{18,21}]tricosa- | |
| | 1(20),2(23),3,5,15(22),16,18(21)-heptaene | |
| Example 34 | (13S)-4-(3-methoxyazetidin-1-yl)-13-methyl-7,10- | Scheme E |
| | dioxa-5,14,19,20,23- | |
| | pentaazatetracyclo[13.5.2.1^{2,6}.0^{18,21}]tricosa- | |
| | 1(20),2,4,6(23),15(22),16,18(21)-heptaene | |
| Example 35 | (6R,12R)-6,12-dimethyl-9,13-dioxa-4,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 36 | (6S,12R)-6,12-dimethyl-9,13-dioxa-4,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 37 | (6S)-6-methyl-9,13-dioxa-4,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 38 | (6S,12S)-6,12-dimethyl-9,13-dioxa-4,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 39 | (13S)-13-methyl-4-(pyrrolidin-1-yl)-7,10-dioxa- | Scheme E |
| | 5,14,19,20,23-pentaazatetracyclo[13.5.2.1^{2,6}.0^{18,21}] | |
| | tricosa-1(20),2(23),3,5,15(22),16,18(21)-heptaene | |
| Example 40 | (13S)-4,13-dimethyl-7,10-dioxa-5,14,19,20,23- | Scheme E |
| | pentaazatetracyclo[13.5.2.1^{2,6}.0^{18,21}]tricosa- | |
| | 1(20),2(23),3,5,15(22),16,18(21)-heptaene | |
| Example 41 | (13R)-13-methyl-7,10,14-trioxa-4,19,20,23- | Scheme D |
| | tetraazatetracyclo[13.5.2.1^{2,6}.0^{18,21}]tricosa- | |
| | 1(20),2(23),3,5,15(22),16,18(21)-heptaene | |
| Example 42 | (12R)-12-methyl-9,13-dioxa-4,5,18,19- | Scheme F |
| | tetraazatetracyclo[12.5.2.1^{2,5}.0^{17,20}]docosa- | |
| | 1(19),2(22),3,14(21),15,17(20)-hexaene | |
| Example 43 | (13S)-13-methyl-10,14-dioxa-19,20- | Scheme D |
| | diazatetracyclo[13.5.2.1^{2,6}.0^{18,21}]tricosa- | |
| | 1(20),2(23),3,5,15(22),16,18(21)-heptaene | |
| Example 44 | (8R,13S)-4-(3-methoxyazetidin-1-yl)-8,13-dimethyl- | Scheme C |
| | 7,10,14-trioxa-5,19,20,23- | |
| | tetraazatetracyclo[13.5.2.1^{2,6}.0^{18,21}]tricosa- | |
| | 1(20),2(23),3,5,15(22),16,18(21)-heptaene | |
| Example 45 | (13R)-13-methyl-7,10,14-trioxa-5,19,20,23- | Scheme C |
| | tetraazatetracyclo[13.5.2.1^{2,6}.0^{18,21}]tricosa- | |
| | 1(20),2(23),3,5,15(22),16,18(21)-heptaene | |
| Example 46 | (8S,13S)-8,13-dimethyl-7,10,14-trioxa-5,19,20,23- | Scheme C |
| | tetraazatetracyclo[13.5.2.1^{2,6}.0^{18,21}]tricosa- | |
| | 1(20),2(23),3,5,15(22),16,18(21)-heptaene | |
| Example 47 | (8S,13S)-8,13-dimethyl-7,10,14-trioxa-4,19,20,23- | Scheme D |
| | tetraazatetracyclo[13.5.2.1^{2,6}.0^{18,21}]tricosa- | |
| | 1(20),2(23),3,5,15(22),16,18(21)-heptaene | |
| Example 48 | (12S)-12-methyl-9,13-dioxa-3-thia-18,19,22- | Scheme D |
| | triazatetracyclo[12.5.2.1^{2,5}.0^{17,20}]docosa- | |
| | 1(19),2(22),4,14(21),15,17(20)-hexaene | |
| Example 49 | (8R,13S)-4-[(3R)-3-methoxypyrrolidin-1-yl]-8,13- | Scheme C |
| | dimethyl-7,10,14-trioxa-5,19,20,23- | |
| | tetraazatetracyclo[13.5.2.1^{2,6}.0^{18,21}]tricosa- | |
| | 1(20),2(23),3,5,15(22),16,18(21)-heptaene | |
| Example 50 | (13R)-13-methyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 51 | (9S,13S)-9,13-dimethyl-7,10,14-trioxa-4,19,20,23- | Scheme D |
| | tetraazatetracyclo[13.5.2.1^{2,6}.0^{18,21}]tricosa- | |
| | 1(20),2(23),3,5,15(22),16,18(21)-heptaene | |
| Example 52 | (7R,12S)-7,12-dimethyl-9,13-dioxa-4,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 53 | (8R,13S)-8,13-dimethyl-7,11,14-trioxa-5,19,20,23- | Scheme C |
| | tetraazatetracyclo[13.5.2.1^{2,6}.0^{18,21}] tricosa- | |
| | 1(20),2(23),3,5,15(22),16,18(21)-heptaene | |
| Example 54 | (12S)-12-methyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 55 | (8R)-8-methyl-7,10,14-trioxa-5,19,20,23- | Scheme G |
| | tetraazatetracyclo[13.5.2.1^{2,6}.0^{18,21}]tricosa- | |
| | 1(20),2(23),3,5,15(22),16,18(21)heptaene | |
| Example 56 | (13R)-13-methyl-7,10,14-trioxa-19,20,23- | Scheme D |
| | triazatetracyclo[13.5.2.1^{2,6}.0^{18,21}]tricosa- | |
| | 1(20),2(23),3,5,15(22),16,18(21)-heptaene | |
| Example 57 | (12S)-12-methyl-9,13-dioxa-3,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 58 | (9R,13S)-9,13-dimethyl-7,10,14-trioxa-4,19,20,23- | Scheme D |
| | tetraazatetracyclo[13.5.2.1^{2,6}.0^{18,21}]tricosa- | |
| | 1(20),2(23),3,5,15(22),16,18(21)-heptaene | |
| Example 59 | (9S,13S)-9,13-dimethyl-7,10,14-trioxa-5,19,20,23- | Scheme C |
| | tetraazatetracyclo[13.5.2.1^{2,6}.0^{18,21}]tricosa- | |
| | 1(20),2(23),3,5,15(22),16,18(21)-heptaene | |
| Example 60 | (9R)-9-methyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 61 | (9S)-9-methyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 62 | (13S)-13-methyl-7,10,14-trioxa-4-thia-19,20,23- | Scheme D |
| | triazatetracyclo[13.5.2.1^{2,5}.0^{18,21}]tricosa- | |
| | 1(20),2,5(23),15(22),16,18(21)-hexaene | |
| Example 63 | 8,11,14-trioxa-4,5,19,20- | Scheme B |
| | tetraazatetracyclo[13.5.2.1^{2,5}.0^{18,21}]tricosa- | |
| | 1(20),2(23),3,15(22),16,18(21)-hexaene | |
| Example 64 | (6S)-6-methyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 65 | (6R)-6-methyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 66 | (13S)-13-methyl-7,10,14-trioxa-23-thia-4,19,20- | Scheme D |
| | triazatetracyclo[13.5.2.1^{2,5}.0^{18,21}]tricosa- | |
| | 1(20),2,4,15(22),16,18(21)-hexaene | |
| Example 67 | (12R)-12-methyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 68 | 9,12,15-trioxa-4,20,21- | Scheme D |
| | triazatetracyclo[14.5.2.1^{2,6}.0^{19,22}]tetracosa- | |
| | 1(21),2(24),3,5,16(23),17,19(22)-heptaene | |
| Example 69 | (14S)-14-methyl-9,12,15-trioxa-4,20,21- | Scheme D |
| | triazatetracyclo[14.5.2.1^{2,6}.0^{19,22}]tetracosa- | |
| | 1(21),2(24),3,5,16(23),17,19(22)-heptaene | |
| Example 70 | (10R)-10-methyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 71 | (13R)-13-methyl-8,11,14-trioxa-4,5,19,20,23- | Scheme D |
| | pentaazatetracyclo[13.5.2.1^{2,5}.0^{18,21}]tricosa- | |
| | 1(20),2(23),3,15(22),16,18(21)-hexaene | |
| Example 72 | (13S)-13-methyl-8,11,14-trioxa-5,19,20-triaza | Scheme D |
| | tetracyclo[13.5.2.1^{2,5}.0^{18,21}]tricosa-1(20),2(23), | |
| | 3,15(22),16,18(21)-hexaene-4-carbonitrile | |
| Example 73 | 8,11,14-trioxa-4,5,19,20,22- | Scheme A |
| | pentaazatetracyclo[13.5.2.1^{2,5}.0^{18,21}]tricosa- | |
| | 1(20),2(23),3,15(22),16,18(21)-hexaene | |
| Example 74 | 9,13-dioxa-4,5,18,19,22- | Scheme D |
| | pentaazatetracyclo[12.5.2.1^{2,5}.0^{17,20}]docosa- | |
| | 1(19),2(22),3,14(21),15,17(20)-hexaene | |
| Example 75 | (13R)-13-methyl-7,10,14-trioxa-23-thia-4,19,20- | Scheme D |
| | triazatetracyclo[13.5.2.1^{2,5}.0^{18,21}]tricosa- | |
| | 1(20),2,4,15(22),16,18(21)-hexaene | |
| Example 76 | 13R)-4,13-dimethyl-8,11,14-trioxa-4,19,20,23- | Scheme D |
| | tetraazatetracyclo[13.5.2.1^{2,5}.0^{18,21}]tricosa- | |
| | 1(20),2,5(23),15(22),16,18(21)-hexaene | |
| Example 77 | (7R)-7-methyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 78 | (13,13-difluoro-8,11,14-trioxa-4,5,19,20- | Scheme F |
| | tetraazatetracyclo[13.5.2.1^{2,5}.0^{18,21}]tricosa- | |
| | 1(20),2(23),3,15(22),16,18(21)-hexaene | |
| Example 79 | (13S)-4,13-dimethyl-7,10,14-trioxa-4,19,20,23- | Scheme D |
| | tetraazatetracyclo[13.5.2.1^{2,5}.0^{18,21}]tricosa- | |
| | 1(20),2,5(23),15(22),16,18(21)-hexaene | |
| Example 80 | (13S)-13-methyl-8,11,14-trioxa-5,19,20,23- | Scheme D |
| | tetraazatetracyclo[13.5.2.1^{2,5}.0^{18,21}]tricosa- | |
| | 1(20),2(23),3,15(22),16,18(21)-hexaene | |
| Example 81 | (13S)-13-methyl-8,11,14-trioxa-4,5,19,20,23- | Scheme D |
| | pentaazatetracyclo[13.5.2.1^{2,5}.0^{18,21}]tricosa- | |
| | 1(20),2(23),3,15(22),16,18(21)-hexaene | |
| Example 82 | (13R)-13-methyl-8,11,14-trioxa-5,19,20-triaza | Scheme D |
| | tetracyclo[13.5.2.1^{2,5}.0^{18,21}]tricosa-1(20),2(23), | |
| | 3,15(22),16,18(21)-hexaene-4-carbonitrile | |
| Example 83 | (13R)-13-methyl-8,11,14-trioxa-5,19,20,23- | Scheme D |
| | tetraazatetracyclo[13.5.2.1^{2,5}.0^{18,21}]tricosa- | |
| | 1(20),2(23),3,15(22),16,18(21)-hexaene | |
| Example 84 | (13S)-4,13-dimethyl-7,10,14-trioxa-4,19,20,23- | Scheme D |
| | tetraazatetracyclo[13.5.2.1^{2,5}.0^{18,21}]tricosa- | |
| | 1(20),2,5(23),15(22),16,18(21)-hexaene | |
| Example 85 | (12S)-12-methyl-8,11,14-trioxa-5,19,20-triaza | Scheme D |
| | tetracyclo[13.5.2.1^{2,5}.0^{18,21}]tricosa-1(20),2(23), | |
| | 3,15(22),16,18(21)-hexaene-4-carbonitrile | |
| Example 86 | (12R)-12-methyl-8,11,14-trioxa-5,19,20-triaza | Scheme D |
| | tetracyclo[13.5.2.1^{2,5}.0^{18,21}]tricosa-1(20),2(23), | |
| | 3,15(22),16,18(21)-hexaene-4-carbonitrile | |
| Example 87 | (6R,12S)-6,12-dimethyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 88 | (13R)-17-fluoro-13-methyl-8,11,14-trioxa-4,5,19,20- | Scheme B |
| | tetraazatetracyclo[13.5.2.1^{2,5}.0^{18,21}]tricosa- | |
| | 1(20),2(23),3,15(22),16,18(21)-hexaene | |
| Example 89 | (6S,12S)-6,12-dimethyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 90 | (12R)-12-methyl-9,13-dioxa-4,5,18,19,22- | Scheme D |
| | pentaazatetracyclo[12.5.2.1^{2,5}.0^{17,20}]docosa- | |
| | 1(19),2(22),3,14(21),15,17(20)-hexaene | |
| Example 91 | 13-methyl-4-(morpholin-4-yl)-7,10-dioxa-13,19,20- | Scheme H |
| | triazatetracyclo[13.5.2.1^{2,6}.0^{18,21}]tricosa- | |
| | 1(20),2,4,6(23),15,17,21-heptaene | |
| Example 92 | (12S)-12-methyl-9,13-dioxa-4,5,18,19,22- | Scheme D |
| | pentaazatetracyclo[12.5.2.1^{2,5}.0^{17,20}]docosa- | |
| | 1(19),2(22),3,14(21),15,17(20)-hexaene | |
| Example 93 | (6S,13R)-6,13-dimethyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 94 | (6R,12R)-6,12-dimethyl-9,13-dioxa-4,5,18,19,22- | Scheme D |
| | pentaazatetracyclo[12.5.2.1^{2,5}.0^{17,20}]docosa- | |
| | 1(19),2(22),3,14(21),15,17(20)-hexaene | |
| Example 95 | (13R)-6-methoxy-13-methyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 96 | 8,11,14-trioxa-4,5,16,19,20- | Synthesis |
| | pentaazatetracyclo[13.5.2.1^{2,5}.0^{18,21}]tricosa- | described |
| | 1(20),2(23),3,15(22),16,18(21)-hexaene | in detail |
| Example 97 | (8S,12R)-8,12-dimethyl-9,13-dioxa-4,5,18,19-tetraaza | Scheme E |
| | tetracyclo[12.5.2.1^{2,5}.0^{17,20}]docosa- | |
| | 1(19),2(22),3,14(21),15,17(20)-hexaene | |
| Example 98 | (8R,12R)-8,12-dimethyl-9,13-dioxa-4,5,18,19- | Scheme E |
| | tetraazatetracyclo[12.5.2.1²,⁵.0¹⁷,²⁰]docosa- | |
| | 1(19),2(22),3,14(21),15,17(20)-hexaene | |
| Example 99 | (12S,13R)-12,13-dimethyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 100 | (12R,13S)-12,13-dimethyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 101 | (6S,13S)-6,13-dimethyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 102 | 8,13-dioxa-4,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 103 | (6S,12R)-6,12-dimethyl-9,13-dioxa-4,5,18,19,22- | Scheme D |
| | pentaazatetracyclo[12.5.2.1^{2,5}.0^{17,20}]docosa- | |
| | 1(19),2(22),3,14(21),15,17(20)-hexaene | |
| Example 104 | 10,13-dioxa-4,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 105 | (6R*,13R)-6-fluoro-13-methyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 106 | (6S*,13R)-6-fluoro-13-methyl-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 107 | (12S)-12-methyl(9,9,10,10-²H₄)-8,11,14-trioxa- | Scheme D |
| | 4,5,19,20-tetraazatetracyclo[13.5.2.1^{2,5}.0^{18,21}]tricosa- | |
| | 1(20),2(23),3,15,17,21-hexaene | |
| Example 108 | 8,14-dioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 109 | (6R)-11,11-difluoro-6-methyl-9,13-dioxa-4,5,18,19- | Scheme E |
| | tetraazatetracyclo[12.5.2.1^{2,5}.0^{17,20}]docosa- | |
| | 1(19),2(22),3,14(21),15,17(20)-hexaene | |
| Example 110 | (12S)-12-methyl(6,6,7,7-2H4)-8,11,14-trioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 111 | 11,11-difluoro-9,13-dioxa-4,5,18,19- | Scheme E |
| | tetraazatetracyclo[12.5.2.1^{2,5}.0^{17,20}]docosa- | |
| | 1(19),2(22),3,14(21),15,17(20)-hexaene | |
| Example 112 | (12S)-12-methyl-11,14-dioxa-4,5,19,20- | Scheme D |
| | tetraazatetracyclo[13.5.2.1^{2,5}.0^{18,21}]tricosa- | |
| | 1(20),2(23),3,15(22),16,18(21)-hexaene | |
| Example 113 | (12R,13R)-12,13-dimethyl-8,11,14-trioxa-4,5,19,20- | Scheme E |
| | tetraazatetracyclo[13.5.2.1^{2,5}.0^{18,21}]tricosa- | |
| | 1(20),2(23),3,15(22),16,18(21)-hexaene | |
| Example 114 | (12S,13S)-12,13-dimethyl-8,11,14-trioxa-4,5,19,20- | Scheme E |
| | tetraazatetracyclo[13.5.2.1^{2,5}.0^{18,21}]tricosa- | |
| | 1(20),2(23),3,15(22),16,18(21)-hexaene | |
| Example 115 | 8,13-dioxa-4,5,18,19,22- | Scheme D |
| | pentaazatetracyclo[12.5.2.1^{2,5}.0^{17,20}]docosa- | |
| | 1(19),2(22),3,14(21),15,17(20)-hexaene | |
| Example 116 | (6R,10S)-6,10-dimethyl-9,13-dioxa-4,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 117 | (6R,8R)-6,8-dimethyl-9,13-dioxa-4,5,18,19- | Scheme E |
| | tetraazatetracyclo[12.5.2.1^{2,5}.0^{17,20}]docosa- | |
| | 1(19),2(22),3,14(21),15,17(20)-hexaene | |
| Example 118 | 7,7-difluoro-9,13-dioxa-4,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 119 | (13S)-4,13-dimethyl-7,11,14-trioxa-4,19,20,23- | Scheme D |
| | tetraazatetracyclo[13.5.2.1^{2,5}.0^{18,21}]tricosa- | |
| | 1(20),2,5(23),15(22),16,18(21)-hexaene | |
| Example 120 | (12R)-7,7-difluoro-12-methyl-9,13-dioxa-4,5,18,19- | Scheme D |
| | tetraazatetracyclo[12.5.2.1^{2,5}.0^{17,20}]docosa- | |
| | 1(19),2(22),3,14(21),15,17(20)-hexaene | |
| Example 121 | (8E)-11,14-dioxa-4,5,19,20- | Synthesis |
| | tetraazatetracyclo[13.5.2.1^{2,5}.0^{18,21}]tricosa- | described |
| | 1(20),2(23),3,8,15(22),16,18(21)-heptaene | in details |
| Example 122 | (12S)-12-methyl-8,13-dioxa-4,5,18,19,22- | Scheme D |
| | pentaazatetracyclo[12.5.2.1^{2,5}.0^{17,20}]docosa- | |
| | 1(19),2(22),3,14(21),15,17(20)-hexaene | |
| Example 123 | (6R,10R)-6,10-dimethyl-9,13-dioxa-4,5,18,19- | Scheme E |
| | tetraazatetracyclo[12.5.2.1^{2,5}.0^{17,20}]docosa- | |
| | 1(19),2(22),3,14(21),15,17(20)-hexaene | |
| Example 124 | (12R)-12-methyl-8,13-dioxa-4,5,18,19,22- | Scheme D |
| | pentaazatetracyclo[12.5.2.1^{2,5}.0^{17,20}]docosa- | |
| | 1(19),2(22),3,14(21),15,17(20)-hexaene | |
| Example 125 | (12R)-12-methyl-9,13-dioxa-2,4,18,19,22- | Synthesis |
| | pentaazatetracyclo[12.5.2.1^{2,5}.0^{17,20}]docosa- | described |
| | 1(19),3,5(22),14(21),15,17(20)-hexaene | in details |
| Example 126 | (10R,12R)-10,12-dimethyl-9,13-dioxa-4,5,18,19- | Scheme E |
| | tetraazatetracyclo[12.5.2.1^{2,5}.0^{17,20}]docosa- | |
| | 1(19),2(22),3,14(21),15,17(20)-hexaene | |
| Example 127 | (8R,12R)-8,12-dimethyl-9,13-dioxa-4,5,18,19,22- | Scheme E |
| | pentaazatetracyclo[12.5.2.1^{2,5}.0^{17,20}]docosa- | |
| | 1(19),2(22),3,14(21),15,17(20)-hexaene | |
| Example 128 | (6R,12R)-6,12-dimethyl-8,13-dioxa-4,5,18,19- | Scheme E |
| | tetraazatetracyclo[12.5.2.1^{2,5}.0^{17,20}]docosa- | |
| | 1(19),2(22),3,14(21),15,17(20)-hexaene | |
| Example 129 | (13S)-13-methyl-9,12,15-trioxa-4,5,20,21- | Scheme E |
| | tetraazatetracyclo[14.5.2.1^{2,5}.0^{19,22}]tetracosa- | |
| | 1(21),2(24),3,16(23),17,19(22)-hexaene | |
| Example 130 | (12R)-12-methyl-10,13-dioxa-4,5,18,19,22- | Scheme D |
| | pentaazatetracyclo[12.5.2.1^{2,5}.0^{17,20}]docosa- | |
| | 1(19),2(22),3,14(21),15,17(20)-hexaene | |
| Example 131 | (14R)-14-methyl-8,11,15-trioxa-4,5,20,21- | Scheme E |
| | tetraazatetracyclo[14.5.2.1^{2,5}.0^{19,22}]tetracosa- | |
| | 1(21),2(24),3,16(23),17,19(22)-hexaene | |
| Example 132 | (6R,8S)-6,8-dimethyl-9,13-dioxa-4,5,18,19- | Scheme E |
| | tetraazatetracyclo[12.5.2.1^{2,5}.0^{17,20}]docosa- | |
| | 1(19),2(22),3,14(21),15,17(20)-hexaene | |
| Example 133 | (14R)-14-methyl-8,11,15-trioxa-4,5,20,21,24- | Scheme E |
| | pentaazatetracyclo[14.5.2.1^{2,5}.0^{19,22}]tetracosa- | |
| | 1(21),2(24),3,16(23),17,19(22)-hexaene | |
| Example 134 | 7,14-dioxa-10,19,20- | Scheme I |
| | triazatetracyclo[13.5.2.1^{2,6}.0^{18,21}]tricosa- | |
| | 1(20),2(23),3,5,15(22),16,18(21)-heptaen-9-one | |
| Example 135 | 7,14-dioxa-10,19,20- | Scheme I |
| | triazatetracyclo[13.5.2.1^{2,6}.0^{18,21}]tricosa- | |
| | 1(20),2(23),3,5,15(22),16,18(21)-heptaene | |
| Example 136 | 7,14-dioxa-4,10,19,20- | Scheme I |
| | tetraazatetracyclo[13.5.2.1^{2,6}.0^{18,21}]tricosa- | |
| | 1(20),2,4,6(23),15,17,21-heptaen-9-one | |
| Example 137 | 7,14-dioxa-4,10,19,20- | Scheme I |
| | tetraazatetracyclo[13.5.2.1^{2,6}.0^{18,21}]tricosa- | |
| | 1(20),2,4,6(23),15,17,21-heptaene | |
| Example 138 | 5-fluoro-7,14-dioxa-10,19,20- | Scheme I |
| | triazatetracyclo[13.5.2.1^{2,6}.0^{18,21}]tricosa- | |
| | 1(20),2,4,6(23),15,17,21-heptaen-9-one | |
| Example 139 | 5-fluoro-7,14-dioxa-10,19,20- | Scheme I |
| | triazatetracyclo[13.5.2.1^{2,6}.0^{18,21}]tricosa- | |
| | 1(20),2,4,6(23),15,17,21-heptaene | |
| Example 140 | 5-methyl-7,14-dioxa-10,19,20- | Scheme I |
| | triazatetracyclo[13.5.2.1^{2,6}.0^{18,21}]tricosa- | |
| | 1(20),2,4,6(23),15,17,21-heptaen-9-one | |
| Example 141 | 5-methyl-7,14-dioxa-10,19,20- | Scheme I |
| | triazatetracyclo[13.5.2.1^{2,6}.0^{18,21}]tricosa- | |
| | 1(20),2,4,6(23),15,17,21-heptaene | |
| Example 142 | 4-chloro-7,14-dioxa-10,19,20- | Scheme I |
| | triazatetracyclo[13.5.2.1^{2,6}.0^{18,21}]tricosa- | |
| | 1(20),2,4,6(23),15,17,21-heptaen-9-one | |
| Example 143 | 4-chloro-7,14-dioxa-10,19,20- | Scheme I |
| | triazatetracyclo[13.5.2.1^{2,6}.0^{18,21}]tricosa- | |
| | 1(20),2,4,6(23),15,17,21-heptaene | |
| Example 144 | 4-methyl-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 145 | 4-methyl-7,14-dioxa-10,19,20- | Scheme I |
| | triazatetracyclo[13.5.2.1^{2,6}.0^{18,21}]tricosa- | |
| | 1(20),2(23),3,5,15,17,21-heptaene | |
| Example 146 | 4-(oxan-4-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 147 | 4-(oxan-4-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaene | |
| Example 148 | 4-(oxan-3-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 149 | 4-(oxan-3-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaene | |
| Example 150 | (11S)or(11R)-11-(methoxymethyl)-7,14-dioxa- | Scheme I |
| | 10,19,20-triazatetracyclo[13.5.2.1^{2,6}.0^{18,21}]tricosa- | |
| | 1(20),2(23),3,5,15,17,21-heptaen-9-one | |
| Example 151 | (11S)or(11R)-11-(methoxymethyl)-7,14-dioxa- | Scheme I |
| | 10,19,20-triazatetracyclo[13.5.2.1^{2,6}.0^{18,21}]tricosa- | |
| | 1(20),2(23),3,5,15,17,21-heptaene | |
| Example 152 | (11R)or(11S)-11-(methoxymethyl)-7,14-dioxa- | Scheme I |
| | 10,19,20-triazatetracyclo[13.5.2.1^{2,6}.0^{18,21}]tricosa- | |
| | 1(20),2(23),3,5,15,17,21-heptaen-9-one | |
| Example 153 | (11R)or(11S)-11-(methoxymethyl)-7,14-dioxa- | Scheme I |
| | 10,19,20-triazatetracyclo[13.5.2.1^{2,6}.0^{18,21}]tricosa- | |
| | 1(20),2(23),3,5,15,17,21-heptaene | |
| Example 154 | 11-(fluoromethyl)-7,14-dioxa-10,19,20- | Scheme I |
| | triazatetracyclo[13.5.2.1^{2,6}.0^{18,21}]tricosa- | |
| | 1(20),2,4,6(23),15,17,21-heptaen-9-one | |
| Example 155 | 11-(fluoromethyl)-7,14-dioxa-10,19,20- | Scheme I |
| | triazatetracyclo[13.5.2.1^{2,6}.0^{18,21}]tricosa- | |
| | 1(20),2,4,6(23),15,17,21-heptaene | |
| Example 156 | 7′,14′-dioxa-4′,10′,19′,20′-tetraazaspiro[cyclopropane- | Scheme I |
| | 1,8′-tetracyclo[13.5.2.1^{2,6}.0^{18,21}]tricosane]- | |
| | 1′(20′),2′(23′),3′,5′,15′,17′,21′-heptaen-9′-one | |
| Example 157 | 7′,14′-dioxa-4′,10′,19′,20′-tetraazaspiro[cyclopropane- | Scheme J |
| | 1,8′-tetracyclo[13.5.2.1^{2,6}.0^{18,21}]tricosane]- | |
| | 1′(20′),2′(23′),3′,5′,15′,17′,21′-heptaene | |
| Example 158 | 13-methyl-7,14-dioxa-4,10,19,20- | Scheme I |
| | tetraazatetracyclo[13.5.2.1^{2,6}.0^{18,21}]tricosa- | |
| | 1(20),2,4,6(23),15,17,21-heptaen-9-one | |
| Example 159 | 13-methyl-7,14-dioxa-4,10,19,20- | Scheme I |
| | tetraazatetracyclo[13.5.2.1^{2,6}.0^{18,21}]tricosa- | |
| | 1(20),2,4,6(23),15,17,21-heptaene | |
| Example 160 | 8,10-dimethyl-7,14-dioxa-4,10,19,20- | Scheme I |
| | tetraazatetracyclo[13.5.2.1^{2,6}.0^{18,21}]tricosa- | |
| | 1(20),2,4,6(23),15,17,21-heptaen-9-one | |
| Example 161 | 8,10-dimethyl-7,14-dioxa-4,10,19,20- | Scheme I |
| | tetraazatetracyclo[13.5.2.1^{2,6}.0^{18,21}]tricosa- | |
| | 1(20),2,4,6(23),15,17,21-heptaene | |
| Example 162 | (13R)-13-methyl-7,14-dioxa-4,10,19,20- | Scheme I |
| | tetraazatetracyclo[13.5.2.1^{2,6}.0^{18,21}]tricosa- | |
| | 1(20),2,4,6(23),15(22),16,18(21)-heptaen-9-one | |
| Example 163 | (13R)-13-methyl-7,14-dioxa-4,10,19,20- | Scheme I |
| | tetraazatetracyclo[13.5.2.1^{2,6}.0^{18,21}]tricosa- | |
| | 1(20),2,4,6(23),15(22),16,18(21)-heptaene | |
| Example 164 | (7S,9S)-15-oxa-11,20,21- | Scheme I |
| | triazapentacyclo[14.5.2.1^{2,6}.0^{7,9}.0^{19,22}]tetracosa- | |
| | 1(21),2(24),3,5,16(23),17,19(22)-heptaen-10-one | |
| Example 165 | (7S,9S)-15-oxa-11,20,21- | Scheme I |
| | triazapentacyclo[14.5.2.1^{2,6}.0^{7,9}.0^{19,22}]tetracosa- | |
| | 1(21),2(24),3,5,16(23),17,19(22)-heptaene | |
| Example 166 | 14-oxa-10,19,20- | Scheme I |
| | triazatetracyclo[13.5.2.1^{2,6}.0^{18,21}]tricosa- | |
| | 1(20),2(23),3,5,15(22),16,18(21)-heptaen-9-one | |
| Example 167 | 14-oxa-10,19,20- | Scheme I |
| | triazatetracyclo[13.5.2.1^{2,6}.0^{18,21}]tricosa- | |
| | 1(20),2(23),3,5,15(22),16,18(21)-heptaene | |
| Example 168 | (11S,12S)-11,12-dimethyl-7,14-dioxa-10,19,20- | Scheme I |
| | triazatetracyclo[13.5.2.1^{2,6}.0^{18,21}]tricosa- | |
| | 1(20),2(23),3,5,15(22),16,18(21)-heptaen-9-one | |
| Example 169 | (11S,12S)-11,12-dimethyl-7,14-dioxa-10,19,20- | Scheme I |
| | triazatetracyclo[13.5.2.1^{2,6}.0^{18,21}]tricosa- | |
| | 1(20),2(23),3,5,15(22),16,18(21)-heptaene | |
| Example 170 | 12-methoxy-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 171 | 12-methoxy-7,14-dioxa-10,19,20- | Scheme I |
| | triazatetracyclo[13.5.2.1^{2,6}.0^{18,21}]tricosa- | |
| | 1(20),2(23),3,5,15,17,21-heptaene | |
| Example 172 | (13R)-13-methyl-4-(morpholin-4-yl)-7,14-dioxa- | Scheme I |
| | 10,19,20-triazatetracyclo[13.5.2.1^{2,6}.0^{18,21}]tricosa- | |
| | 1(20),2(23),3,5,15,17,21-heptaen-9-one | |
| Example 173 | (13R)-13-methyl-4-(morpholin-4-yl)-7,14-dioxa- | Scheme I |
| | 10,19,20-triazatetracyclo[13.5.2.1^{2,6}.0^{18,21}]tricosa- | |
| | 1(20),2(23),3,5,15,17,21-heptaene | |
| Example 174 | (13S)-13-methyl-4-(morpholin-4-yl)-7,14-dioxa- | Scheme I |
| | 10,19,20-triazatetracyclo[13.5.2.1^{2,6}.0^{18,21}]tricosa- | |
| | 1(20),2(23),3,5,15,17,21-heptaen-9-one | |
| Example 175 | (13S)-13-methyl-4-(morpholin-4-yl)-7,14-dioxa- | Scheme I |
| | 10,19,20-triazatetracyclo[13.5.2.1^{2,6}.0^{18,21}]tricosa- | |
| | 1(20),2(23),3,5,15,17,21-heptaene | |
| Example 176 | 8-methyl-4-(morpholin-4-yl)-7,14-dioxa-10,19,20- | Scheme I |
| | triazatetracyclo[13.5.2.1^{2,6}.0^{18,21}]tricosa- | |
| | 1(20),2,4,6(23),15,17,21-heptaen-9-one | |
| Example 177 | (8R) or (8S)-8-methyl-4-(morpholin-4-yl)-7,14-dioxa- | Scheme I |
| | 10,19,20-triazatetracyclo[13.5.2.1^{2,6}.0^{18,21}]tricosa- | |
| | 1(20),2,4,6(23),15,17,21-heptaene | |
| Example 178 | (8S) or (8R)-8-methyl-4-(morpholin-4-yl)-7,14-dioxa- | Scheme I |
| | 10,19,20-triazatetracyclo[13.5.2.1^{2,6}.0^{18,21}]tricosa- | |
| | 1(20),2,4,6(23),15,17,21-heptaene | |
| Example 179 | (12R)-12-methyl-13-oxa-4,5,9,18,19- | Scheme I |
| | pentaazatetracyclo[12.5.2.1^{2,5}.0^{17,20}]docosa- | |
| | 1(19),2(22),3,14(21),15,17(20)-hexaen-8-one | |
| Example 180 | (12R)-12-methyl-13-oxa-4,5,9,18,19- | Scheme I |
| | pentaazatetracyclo[12.5.2.1^{2,5}.0^{17,20}]docosa- | |
| | 1(19),2(22),3,14(21),15,17(20)-hexaene | |
| Example 181 | 13-oxa-4,5,9,18,19,22- | Scheme I |
| | hexaazatetracyclo[12.5.2.1^{2,5}.0^{17,20}]docosa- | |
| | 1(19),2(22),3,14(21),15,17(20)-hexaen-8-one | |
| Example 182 | 13-oxa-4,5,9,18,19,22- | Scheme I |
| | hexaazatetracyclo[12.5.2.1^{2,5}.0^{17,20}]docosa- | |
| | 1(19),2(22),3,14(21),15,17(20)-hexaene | |
| Example 183 | (11S,12S)-12-fluoro-11-methyl-7,14-dioxa-10,19,20- | Scheme I |
| | triazatetracyclo[13.5.2.1^{2,6}.0^{18,21}]tricosa- | |
| | 1(20),2,4,6(23),15,17,21-heptaen-9-one | |
| Example 184 | (11S,12S)-12-fluoro-11-methyl-7,14-dioxa-10,19,20- | Scheme I |
| | triazatetracyclo[13.5.2.1^{2,6}.0^{18,21}]tricosa- | |
| | 1(20),2,4,6(23),15,17,21-heptaene | |
| Example 185 | 4-(trifluoromethyl)-7,14-dioxa-10,19,20- | Scheme I |
| | triazatetracyclo[13.5.2.1^{2,6}.0^{18,21}]tricosa- | |
| | 1(20),2,4,6(23),15,17,21-heptaen-9-one | |
| Example 186 | 4-(trifluoromethyl)-7,14-dioxa-10,19,20- | Scheme I |
| | triazatetracyclo[13.5.2.1^{2,6}.0^{18,21}]tricosa- | |
| | 1(20),2,4,6(23),15,17,21-heptaene | |
| Example 187 | 16-methyl-7,14-dioxa-4,10,19,20- | Scheme I |
| | tetraazatetracyclo[13.5.2.1^{2,6}.0^{18,21}]tricosa- | |
| | 1(20),2(23),3,5,15(22),16,18(21)-heptaen-9-one | |
| Example 188 | 16-methyl-7,14-dioxa-4,10,19,20- | Scheme I |
| | tetraazatetracyclo[13.5.2.1^{2,6}.0^{18,21}]tricosa- | |
| | 1(20),2(23),3,5,15(22),16,18(21)-heptaene | |
| Example 189 | 10-methyl-7,14-dioxa-5,10,19,20- | Scheme I |
| | tetraazatetracyclo[13.5.2.1^{2,6}.0^{18,21}]tricosa- | |
| | 1(20),2(23),3,5,15(22),16,18(21)-heptaen-9-one | |
| Example 190 | 10-methyl-7-oxa-4,10,14,19,20- | Scheme I |
| | pentaazatetracyclo[13.5.2.1^{2,6}.0^{18,21}]tricosa- | |
| | 1(20),2,4,6(23),15,17,21-heptaen-9-one | |
| Example 191 | 10-methyl-9-oxo-7,14-dioxa-10,19,20- | Scheme I |
| | triazatetracyclo[13.5.2.1^{2,6}.0^{18,21}]tricosa- | |
| | 1(20),2,4,6(23),15,17,21-heptaene-4-carbonitrile | |
| Example 192 | 7,13-dioxa-10,18,19- | Scheme I |
| | triazatetracyclo[12.5.2.1^{2,6}.0^{17,20}]docosa- | |
| | 1(19),2(22),3,5,14(21),15,17(20)-heptaen-9-one | |
| Example 193 | 7,14-dioxa-4,10,19,20,23- | Scheme I |
| | pentaazatetracyclo[13.5.2.1^{2,6}.0^{18,21}]tricosa- | |
| | 1(20),2(23),3,5,15(22),16,18(21)-heptaen-9-one | |
| Example 194 | 10-methyl-7,14-dioxa-3,10,19,20- | Scheme I |
| | tetraazatetracyclo[13.5.2.1^{2,6}.0^{18,21}]tricosa- | |
| | 1(20),2(23),3,5,15(22),16,18(21)-heptaen-9-one | |
| Example 195 | 4-fluoro-7,14-dioxa-10,19,20- | Scheme I |
| | triazatetracyclo[13.5.2.1^{2,6}.0^{18,21}]tricosa- | |
| | 1(20),2(23),3,5,15(22),16,18(21)-heptaen-9-one | |
| Example 196 | 7,14-dioxa-5,10,19,20,23- | Scheme I |
| | pentaazatetracyclo[13.5.2.1^{2,6}.0^{18,21}]tricosa- | |
| | 1(20),2(23),3,5,15(22),16,18(21)-heptaen-9-one | |
| Example 197 | 8,8,10-trimethyl-7,14-dioxa-4,10,19,20- | Scheme I |
| | tetraazatetracyclo[13.5.2.1^{2,6}.0^{18,21}]tricosa- | |
| | 1(20),2(23),3,5,15(22),16,18(21)-heptaen-9-one | |
| Example 198 | 11-methyl-7,15-dioxa-4,11,20,21- | Scheme I |
| | tetraazatetracyclo[14.5.2.1^{2,6}.0^{19,22}]tetracosa- | |
| | 1(21),2,4,6(24),16,18,22-heptaen-10-one | |
| Example 199 | 7,13-dioxa-4,10,18,19- | Scheme I |
| | tetraazatetracyclo[12.5.2.1^{2,6}.0^{17,20}]docosa- | |
| | 1(19),2(22),3,5,14(21),15,17(20)-heptaen-9-one | |
| Example 200 | 3-fluoro-10-methyl-7,14-dioxa-4,10,19,20- | Scheme I |
| | tetraazatetracyclo[13.5.2.1^{2,6}.0^{18,21}]tricosa- | |
| | 1(20),2,4,6(23),15(22),16,18(21)-heptaen-9-one | |
| Example 201 | 8,8-dimethyl-7,14-dioxa-4,10,19,20- | Scheme I |
| | tetraazatetracyclo[13.5.2.1^{2,6}.0^{18,21}]tricosa- | |
| | 1(20),2(23),3,5,15,17,21-heptaen-9-one | |
| Example 202 | 12,12-difluoro-7,14-dioxa-4,10,19,20- | Scheme I |
| | tetraazatetracyclo[13.5.2.1^{2,6}.0^{18,21}]tricosa- | |
| | 1(20),2,4,6(23),15,17,21-heptaen-9-one | |
| Example 203 | 4-bromo-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 204 | 4-(piperidin-1-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 205 | 4-(pyrrolidin-1-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 206 | 4-(3,3-difluoropyrrolidin-1-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 207 | 4-(morpholin-4-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 208 | 4-(3,6-dihydro-2H-pyran-4-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 209 | 4-[4-(cyclopropylmethyl)piperazin-1-yl]-7,14-dioxa- | Scheme I |
| | 10,19,20-triazatetracyclo[13.5.2.1^{2,6}.0^{18,21}]tricosa- | |
| | 1(20),2(23),3,5,15,17,21-heptaen-9-one | |
| Example 210 | 4-(5,6-dihydro-2H-pyran-3-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 211 | 4-(2,5-dihydrofuran-3-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 212 | 4-(piperazin-1-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 213 | 4-(furan-3-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 214 | 4-(oxolan-3-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 215 | (11R,12R)-11,12-dimethyl-7,14-dioxa-10,19,20- | Scheme I |
| | triazatetracyclo[13.5.2.1^{2,6}.0^{18,21}]tricosa- | |
| | 1(20),2,4,6(23),15(22),16,18(21)-heptaen-9-one | |
| Example 216 | 4-[(3R,4R)-3,4-difluoropyrrolidin-1-yl]-7,14-dioxa- | Scheme I |
| | 10,19,20-triazatetracyclo[13.5.2.1^{2,6}.0^{18,21}]tricosa- | |
| | 1(20),2(23),3,5,15,17,21-heptaen-9-one | |
| Example 217 | 4-(4-cyclopropylpiperazin-1-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 218 | 4-(oxan-2-yl)-7,14-dioxa-10,19,20- | Scheme I |
| | triazatetracyclo[13.5.2.1^{2,6}.0^{18,21}]tricosa- | |
| | 1(20),2(23),3,5,15,17,21-heptaen-9-one | |
| Example 219 | 4-fluoro-14-oxa-10,19,20- | Scheme I |
| | triazatetracyclo[13.5.2.1^{2,6}.0^{18,21}]tricosa- | |
| | 1(20),2(23),3,5,15(22),16,18(21)-heptaen-9-one | |
| Example 220 | 14-oxa-4,10,19,20- | Scheme I |
| | tetraazatetracyclo[13.5.2.1^{2,6}.0^{18,21}]tricosa- | |
| | 1(20),2(23),3,5,15(22),16,18(21)-heptaen-9-one | |
| Example 221 | 11-methyl-7,14-dioxa-11,19,20- | Scheme I |
| | triazatetracyclo[13.5.2.1^{2,6}.0^{18,21}]tricosa- | |
| | 1(20),2(23),3,5,15,17,21-heptaen-10-one | |
| Example 222 | 8,14-dioxa-4,5,11,19,20,23- | Scheme I |
| | hexaazatetracyclo[13.5.2.1^{2,5}.0^{18,21}]tricosa- | |
| | 1(20),2(23),3,15(22),16,18(21)-hexaen-10-one | |
| Example 223 | (12R)-12-methyl-13-oxa-4,5,9,18,19,22- | Scheme I |
| | hexaazatetracyclo[12.5.2.1^{2,5}.0^{17,20}]docosa- | |
| | 1(19),2(22),3,14(21),15,17(20)-hexaen-8-one | |
| Example 224 | (12R)-12-methyl-13-oxa-4,5,9,18,19,22- | Scheme I |
| | hexaazatetracyclo[12.5.2.1^{2,5}.0^{17,20}]docosa- | |
| | 1(19),2(22),3,14(21),15,17(20)-hexaen-8-one | |
| Example 225 | (11S)-11-fluoro-13-oxa-4,5,9,18,19,22- | Scheme I |
| | hexaazatetracyclo[12.5.2.1^{2,5}.0^{17,20}]docosa- | |
| | 1(19),2(22),3,14(21),15,17(20)-hexaen-8-one | |
| Example 226 | (12S)-12-methyl-13-oxa-4,5,9,18,19,22- | Scheme I |
| | hexaazatetracyclo[12.5.2.1^{2,5}.0^{17,20}]docosa- | |
| | 1(19),2(22),3,14(21),15,17(20)-hexaen-8-one | |
| Example 227 | (11R)-11-fluoro-13-oxa-4,5,9,18,19,22- | Scheme I |
| | hexaazatetracyclo[12.5.2.1^{2,5}.0^{17,20}]docosa- | |
| | 1(19),2(22),3,14(21),15,17(20)-hexaen-8-one | |
| Example 228 | (11R,12S)-11,12-dimethyl-7,14-dioxa-10,19,20- | Scheme I |
| | triazatetracyclo[13.5.2.1^{2,6}.0^{18,21}]tricosa- | |
| | 1(20),2,4,6(23),15(22),16,18(21)-heptaene | |
| Example 229 | (8R or 8S,13S)-8,13-dimethyl-7,14-dioxa-4,10,19,20- | Scheme I |
| | tetraazatetracyclo[13.5.2.1^{2,6}.0^{18,21}]tricosa- | |
| | 1(20),2,4,6(23),15(22),16,18(21)-heptaene | |
| Example 230 | (8S or 8R,13S)-8,13-dimethyl-7,14-dioxa-4,10,19,20- | Scheme I |
| | tetraazatetracyclo[13.5.2.1^{2,6}.0^{18,21}]tricosa- | |
| | 1(20),2,4,6(23),15(22),16,18(21)-heptaene | |
| Example 231 | (11R,12R)-12-fluoro-11-methyl-7,14-dioxa-10,19,20- | Scheme I |
| | triazatetracyclo[13.5.2.1^{2,6}.0^{18,21}]tricosa- | |
| | 1(20),2,4,6(23),15(22),16,18(21)-heptaene | |
| Example 232 | (13S)-13-methyl-7,14-dioxa-4,10,19,20- | Scheme I |
| | tetraazatetracyclo[13.5.2.1^{2,6}.0^{18,21}]tricosa- | |
| | 1(20),2,4,6(23),15(22),16,18(21)-heptaene | |
| Example 233 | (12S)-12-methyl-13-oxa-4,5,9,18,19- | Scheme I |
| | pentaazatetracyclo[12.5.2.1^{2,5}.0^{17,20}]docosa- | |
| | 1(19),2(22),3,14(21),15,17(20)-hexaene | |
| Example 234 | (7S or 7R,13R)-7,13-dimethyl-8,14-dioxa-4,11,19,20- | Scheme I |
| | tetraazatetracyclo[13.5.2.1^{2,6}.0^{18,21}]tricosa- | |
| | 1(20),2(23),3,5,15(22),16,18(21)-heptaene | |
| Example 235 | (7R or 7S,13R)-7,13-dimethyl-8,14-dioxa-4,11,19,20- | Scheme I |
| | tetraazatetracyclo[13.5.2.1^{2,6}.0^{18,21}]tricosa- | |
| | 1(20),2(23),3,5,15(22),16,18(21)-heptaene | |
| Example 236 | 4-(morpholin-4-yl)-7,14-dioxa-11,19,20- | Scheme J |
| | triazatetracyclo[13.5.2.1^{2,6}.0^{18,21}]tricosa- | |
| | 1(20),2,4,6(23),15,17,21-heptaene | |
| Example 237 | 7,14-dioxa-10,19,20,23- | Scheme J |
| | tetraazatetracyclo[13.5.2.1^{2,6}.0^{18,21}]tricosa- | |
| | 1(20),2,4,6(23),15,17,21-heptaene | |
| Example 238 | 7,14-dioxa-4,11,19,20- | Scheme J |
| | tetraazatetracyclo[13.5.2.1^{2,6}.0^{18,21}]tricosa- | |
| | 1(20),2,4,6(23),15,17,21-heptaene | |
| Example 239 | 10-methyl-4-(propan-2-yloxy)-7,14-dioxa-10,19,20- | Scheme A |
| | triazatetracyclo[13.5.2.1^{2,6}.0^{18,21}]tricosa- | |
| | 1(20),2,4,6(23),15,17,21-heptaene | |
| Example 240 | 11-methyl-7,14-dioxa-10,19,20,23- | Scheme C |
| | tetraazatetracyclo[13.5.2.1^{2,6}.0^{18,21}]tricosa- | |
| | 1(20),2,4,6(23),15,17,21-heptaene | |
| Example 241 | 11,11-difluoro-13-oxa-4,5,9,18,19,22- | Scheme K |
| | hexaazatetracyclo[12.5.2.1^{2,5}.0^{17,20}]docosa- | |
| | 1(19),2(22),3,14(21),15,17(20)-hexaen-8-one | |
| Example 242 | (6S,12R)-6,12-dimethyl-18-(oxan-2-yl)-13-oxa- | Scheme K |
| | 4,5,9,18,19,22-hexaazatetracyclo[12.5.2.1^{2,5}.0^{17,20}] | |
| | docosa-1(19),2(22),3,14(21),15,17(20)-hexaen-8-one | |
| Example 243 | (6S,12R)-6,9,12-trimethyl-18-(oxan-2-yl)-13-oxa- | Scheme K |
| | 4,5,9,18,19,22-hexaazatetracyclo[12.5.2.1^{2,5}.0^{17,20}] | |
| | docosa-1(19),2(22),3,14(21),15,17(20)-hexaen-8-one | |
| Example 244 | (6S,12R)-6,9,12-trimethyl-13-oxa-4,5,9,18,19,22- | Scheme K |
| | hexaazatetracyclo[12.5.2.1^{2,5}.0^{17,20}]docosa- | |
| | 1(19),2(22),3,14(21),15,17(20)-hexaene | |
| Example 245 | (12R)-12-methyl-9,13-dioxa-4,5,15,18,19- | Synthesis |
| | pentaazatetracyclo[12.5.2.1^{2,5}.0^{17,20}]docosa- | described |
| | 1(19),2(22),3,14(21),15,17(20)-hexaene | in details |
| Example 246 | (12S)-12-methyl-8,11,14-trioxa-4,5,16,19,20- | Synthesis |
| | pentaazatetracyclo[13.5.2.1^{2,5}.0^{18,21}]tricosa- | described |
| | 1(20),2(23),3,15(22),16,18(21)-hexaene | in details |

### Pharmacological Study

### Example A: LRRK2 Kinase Activity Assay

The inhibition of LRRK2 kinase was assessed using human recombinant LRRK2 protein in an in vitro peptide-based kinase assay.

### Protocol

LRRK2 Kinase reactions were carried out in 384-well white polystyrene plates in a final volume of 6 μl using ADP-Glo™ Kinase Assay kit (Promega Corp.). Compound and substrates (LRRKtide peptide and ATP) in assay buffer were first dispensed in wells. Kinase reaction was then started by the addition of human recombinant LRRK2 protein. After 1 h-incubation at 37° C., the enzymatic reactions was stopped by the addition of 6 μl of ADP-Glo Reagent-1 and an additional 40-minutes incubation at 23° C. (residual ATP depletion). A final 30-minutes incubation after 12 μL reagent-2 addition (ADP to ATP conversion and luciferin/luciferase reaction) was performed before luminescent signal acquisition (EnVision™ multimode plate reader—PerkinElmer, Inc.). Data from 10 individual concentrations of tested compounds (N=2) were fitted (XLfit™—ID Business Solutions Ltd) to deliver IC₅₀s (compound concentration leading to 50% inhibition of reference enzymatic activity).

### Compounds

The compounds are dissolved to 5 mM in DMSO. When needed, solutions are sonicated in a bath sonicator.

Table 2 provides the pIC₅₀ values of the compounds according to the invention, obtained using the above mentioned kinase assay. Activities are represented as +++, ++ and +, having the following meanings:
«+++» means an IC50<10 nM
«++» means an IC50<100 nM
«+» means an IC50<1 μM
«−» means an IC50>1 μM

TABLE 2

| | IC50 | | IC50 | | IC50 |
|---|---|---|---|---|---|
| Example | LRRK2 wt | Example | LRRK2 wt | Example | LRRK2 wt |
| number | ADP-Glo | number | ADP-Glo | number | ADP-Glo |
| Example 1 | +++ | Example 2 | +++ | Example 3 | +++ |
| Example 4 | +++ | Example 5 | +++ | Example 6 | +++ |
| Example 7 | +++ | Example 8 | +++ | Example 9 | ++ |
| Example 10 | ++ | Example 11 | ++ | Example 12 | ++ |
| Example 13 | ++ | Example 14 | ++ | Example 15 | +++ |
| Example 16 | +++ | Example 17 | +++ | Example 18 | +++ |
| Example 19 | +++ | Example 20 | ++ | Example 21 | +++ |
| Example 22 | +++ | Example 23 | +++ | Example 24 | +++ |
| Example 25 | ++ | Example 26 | +++ | Example 27 | +++ |
| Example 28 | +++ | Example 29 | +++ | Example 30 | ++ |
| Example 31 | +++ | Example 32 | +++ | Example 33 | ++ |
| Example 34 | +++ | Example 35 | +++ | Example 36 | +++ |
| Example 37 | +++ | Example 38 | +++ | Example 39 | +++ |
| Example 40 | +++ | Example 41 | +++ | Example 42 | +++ |
| Example 43 | +++ | Example 44 | +++ | Example 45 | +++ |
| Example 46 | ++ | Example 47 | ++ | Example 48 | +++ |
| Example 49 | +++ | Example 50 | +++ | Example 51 | ++ |
| Example 52 | +++ | Example 53 | +++ | Example 54 | +++ |
| Example 55 | +++ | Example 56 | +++ | Example 57 | + |
| Example 58 | ++ | Example 59 | ++ | Example 60 | ++ |
| Example 61 | ++ | Example 62 | ++ | Example 63 | +++ |
| Example 64 | +++ | Example 65 | +++ | Example 66 | +++ |
| Example 67 | +++ | Example 68 | ++ | Example 69 | ++ |
| Example 70 | ++ | Example 71 | ++ | Example 72 | +++ |
| Example 73 | ++ | Example 74 | +++ | Example 75 | +++ |
| Example 76 | − | Example 77 | ++ | Example 78 | +++ |
| Example 79 | ++ | Example 80 | + | Example 81 | ++ |
| Example 82 | ++ | Example 83 | + | Example 84 | +++ |
| Example 85 | +++ | Example 86 | +++ | Example 87 | +++ |
| Example 88 | +++ | Example 89 | +++ | Example 90 | +++ |
| Example 91 | ++ | Example 92 | +++ | Example 93 | ++ |
| Example 94 | +++ | Example 95 | ++ | Example 96 | ++ |
| Example 97 | +++ | Example 98 | +++ | Example 99 | +++ |
| Example 100 | ++ | Example 101 | +++ | Example 102 | +++ |
| Example 103 | +++ | Example 104 | +++ | Example 105 | +++ |
| Example 106 | ++ | Example 107 | +++ | Example 108 | +++ |
| Example 109 | ++ | Example 110 | +++ | Example 111 | ++ |
| Example 112 | +++ | Example 113 | +++ | Example 114 | +++ |
| Example 115 | +++ | Example 116 | ++ | Example 117 | ++ |
| Example 118 | +++ | Example 119 | + | Example 120 | +++ |
| Example 121 | +++ | Example 122 | +++ | Example 123 | +++ |
| Example 124 | +++ | Example 125 | +++ | Example 126 | +++ |
| Example 127 | +++ | Example 128 | +++ | Example 129 | ++ |
| Example 130 | ++ | Example 131 | +++ | Example 132 | ++ |
| Example 133 | ++ | Example 134 | ++ | Example 135 | +++ |
| Example 136 | +++ | Example 137 | +++ | Example 138 | ++ |
| Example 139 | +++ | Example 140 | +++ | Example 141 | +++ |
| Example 142 | +++ | Example 143 | +++ | Example 144 | +++ |
| Example 145 | +++ | Example 146 | +++ | Example 147 | +++ |
| Example 148 | +++ | Example 149 | +++ | Example 150 | ++ |
| Example 151 | ++ | Example 152 | ++ | Example 153 | ++ |
| Example 154 | ++ | Example 155 | ++ | Example 156 | ++ |
| Example 157 | +++ | Example 158 | ++ | Example 159 | +++ |
| Example 160 | ++ | Example 161 | ++ | Example 162 | ++ |
| Example 163 | +++ | Example 164 | ++ | Example 165 | + |
| Example 166 | +++ | Example 167 | +++ | Example 168 | ++ |
| Example 169 | ++ | Example 170 | ++ | Example 171 | ++ |
| Example 172 | +++ | Example 173 | +++ | Example 174 | +++ |
| Example 175 | +++ | Example 176 | +++ | Example 177 | +++ |
| Example 178 | +++ | Example 179 | +++ | Example 180 | ++ |
| Example 181 | +++ | Example 182 | +++ | Example 183 | ++ |
| Example 184 | ++ | Example 185 | +++ | Example 186 | +++ |
| Example 187 | ++ | Example 188 | +++ | Example 189 | + |
| Example 190 | ++ | Example 191 | ++ | Example 192 | + |
| Example 193 | +++ | Example 194 | + | Example 195 | +++ |
| Example 196 | +++ | Example 197 | ++ | Example 198 | + |
| Example 199 | ++ | Example 200 | + | Example 201 | +++ |
| Example 202 | +++ | Example 203 | +++ | Example 204 | +++ |
| Example 205 | +++ | Example 206 | +++ | Example 207 | +++ |
| Example 208 | +++ | Example 209 | +++ | Example 210 | +++ |
| Example 211 | +++ | Example 212 | +++ | Example 213 | +++ |
| Example 214 | +++ | Example 215 | ++ | Example 216 | +++ |
| Example 217 | +++ | Example 218 | +++ | Example 219 | +++ |
| Example 220 | +++ | Example 221 | ++ | Example 222 | + |
| Example 223 | +++ | Example 224 | +++ | Example 225 | +++ |
| Example 226 | +++ | Example 227 | +++ | Example 228 | +++ |
| Example 229 | +++ | Example 230 | ++ | Example 231 | ++ |
| Example 232 | +++ | Example 233 | +++ | Example 234 | + |
| Example 235 | ++ | Example 236 | +++ | Example 237 | +++ |
| Example 238 | +++ | Example 239 | ++ | Example 240 | +++ |
| Example 241 | ++ | Example 242 | +++ | Example 243 | ++ |
| Example 244 | ++ | Example 245 | +++ | Example 246 | ++ |

### Example B: Pharmaceutical Composition: Tablets

| | |
|---|---|
| 1000 tablets containing a dose of 5 mg of a | 5 g |
| compound selected from Examples 1 to 246 | |
| Wheat starch | 20 g |
| Maize starch | 20 g |
| Lactose | 30 g |
| Magnesium stearate | 2 g |
| Silica | 1 g |
| Hydroxypropylcellulose | 2 g |

1-77. (canceled)

78. A compound of formula (I): 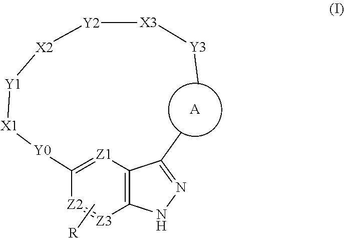
wherein:
R represents a hydrogen atom, a halogen atom or an alkyl group,
Z1, Z2, Z3, each independently represent a carbon or a nitrogen atom, wherein the 6-membered cycle containing Z1, Z2 and 73 may have 0, 1 or 2 nitrogen atoms,
—X— represents —O—, —S—, —S(O)—, S(O)₂— or —N(Ra)—, wherein Ra represents a hydrogen atom or an alkyl group,
—X2- represents —O—, —S—, —S(O), S(O)₂— or —N(Ra)—, wherein Ra represents a hydrogen atom or an alkyl group,
—X3- represents —O—, —S—, —S(O)—, S(O)₂—, —N(Ra)— or a bond, wherein Ra represents a hydrogen atom or an alkyl group,
+Y1- represents a bond or a linear or branched (C₁-C₃) alkanediyl group optionally substituted with one or more substituents, which may be identical or different, selected from a halogen atom, a polyhalogenoalkyl group, an alkoxy group, a hydroxy group, an amino group, an alkylamino group, a dialkylamino group and a cyano group,
Y1- and —Y2-, which may be identical or different, each represent a linear or branched (C₂-C₆) alkanediyl group, a linear or branched (C₂-C₆) alkenediyl group, or a linear or branched (C₃-C₆) cycloalkanediyl group, which groups may be optionally substituted:
by one or more halogen atoms,
or by one substituent selected from a polyhalogenoalkyl group, an alkoxy group, a hydroxy group, an oxo group, an amino group, an alkylamino group, a dialkylamino group and a cyano group,
wherein the carbon atom of the such defined —Y0-, —Y1- or —Y2- groups which is in an alpha position of a heteroatom —X1-, —X2- or —X3-, may not be substituted with an oxygen or a nitrogen heteroatom, unless it is an oxo group,
—Y3- represents a bond, a linear or branched (C₁-C₆) alkanediyl group, a linear or branched (C₂-C₆) alkenediyl group, or a linear or branched (C₃-C₆) cycloalkanediyl group, which groups may be optionally substituted by one or more substituents, which may be identical or different, selected from a halogen atom, a polyhalogenoalkyl group, an alkoxy group, a hydroxy group, an amino group, an alkylamino group, a dialkylamino group and a cyano group,
wherein the carbon atom of the such defined —Y3- group which is in an alpha position to a heteroatom —X3- or A1 when A1 represents a nitrogen atom, may not be substituted with an oxygen or a nitrogen heteroatom,
A represents
an aromatic or partially hydrogenated cyclic group of the formula (a): 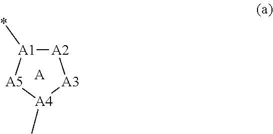
wherein
A1, A4 each independently represent a carbon atom or a nitrogen atom,
A2, A3, A5 each independently represent a carbon atom, an oxygen atom, a sulfur atom or a nitrogen atom,
wherein, when A1 represents a nitrogen atom, it may not be linked to a heteroatom of the —Y0-X1-Y1-X2-Y2-X3-Y3- chain, and
wherein * means that the bond is linked to Y3,
or an aromatic or partially hydrogenated cyclic group of the formula (b): 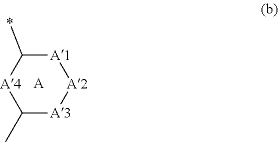
wherein
A′1, A′2, A′3, A′4 each independently represent a carbon atom or a nitrogen atom,
wherein * means that the bond is linked to Y3,
and wherein
the cyclic group A such defined may be optionally substituted by one or more substituents, which may be identical or different, selected from a halogen atom, an alkyl group, an alkoxy group, a hydroxy group, an oxo group, an alkoxyalkyl group, an alkoxyalkoxy group, a polyhalogenoalkyl group, a polyhalogenoalkoxy group, a heterocycloalkyl group, a heterocycloalkylalkyl group, an (alkoxyalkyl)(alkyl)amino group, an amino group, an alkylamino group, a dialkylamino group, a cycloalkyl group, a (heterocycloalkyl)(alkyl)amino group, a dialkylaminoalkyl group, a heterocycloalkylalkoxy group, a cyano group and a cyanoalkyl group,
wherein the heterocycloalkyl and cycloalkyl group may be optionally substituted by one or more substituents selected from an alkyl group, a halogen atom, a polyhalogenoalkyl group, a polyhalogenoalkoxy group, an alkoxy group, an alkoxyalkyl group, a hydroxy group, a cyano group and an oxo group,
its enantiomers, diastereoisomers, tautomers, racemic mixtures, hydrate, solvates, N-oxide, isotopes, deuterated derivatives and addition salts thereof with a pharmaceutically acceptable acid or base.

79. The compound according to claim 78, wherein R represents a hydrogen atom.

80. The compound according to claim 78, wherein R represents a halogen atom.

81. The compound according to claim 78, wherein R represents a methyl group.

82. The compound according to claim 78, wherein Z1, Z2 and Z3 simultaneously represent a carbon atom.

83. The compound according to claim 78, wherein —X1- represents —O—.

84. The compound according to claim 78, wherein —X1- represents —NH—.

85. The compound according to claim 78, wherein —X2- represents —O—.

86. The compound according to claim 78, wherein —X2- represents —NH— or —N(Me)-.

87. The compound according to claim 78, wherein —X3- represents —O—.

88. The compound according to claim 78, wherein —X3- represents a bond.

89. The compound according to claim 78, wherein —Y0- represents a bond.

90. The compound according to claim 78, wherein —Y1- represents a linear or branched alkanediyl group having 2, 3, 4 or 5 carbon atoms.

91. The compound according to claim 90, wherein —Y1- represents —(CH₂)₂—, —CF₂—CH₂—, —CH(CH₂)—CH₂—, —CH₂—CH(CH)—, —CH(CH₃)—CH(CH₃)—, —(CH₂)₃—, —(CH₂)₄—, —CH(CH₃)—(CH₂)₂—,—(CH₂)₂—CH(CH₃)—, —CH₂—CF₂—CH₂—, —CH₂—CHF—CH₂— or —CH(CH₃)—CH₂—CH(CH₃)—.

92. The compound according to claim 78, wherein —Y2- represents a linear or branched alkanediyl group having 2, 3, 4 or 5 carbon atoms.

93. The compound according to claim 92, wherein —Y2- represents —(CH₂)₂—, —(CD₂)₂-, —(CH₂)₃—, —(CD₂)₃-, —CH(CH₃)—CH₂—, —CH₂—CH(CH₃)—, —CH₂—CH(CH₃)—CH₂—, —CH(CH₃)—(CH₂)₂—, —(CH₂)₂—CH(CH₃)—, —CH₂—CF₂—CH₂— or —(CH₂)₄—.

94. The compound according to claim 92, wherein —Y2- represents *—C(O)—(CH₁₂)₂—, *—C(O)—CH₂—CH(CH₃)—, *—C(O)—CH(CH₃) or *—C(O)—CH₂—, wherein * means the link to —X2-.

95. The compound according to claim 78, wherein —Y3- represents a bond.

96. The compound according to claim 78, wherein —Y3- represents a linear or branched alkanediyl group having 1, 2 or 3 carbon atoms.

97. The compound according to claim 9, wherein —Y3- represents —CH₂—, —(CH₂)₂—, —(CD₂)₂-, —CH(CH₃)—, —CH₂—CH(CH₃)—, —CH₂—CHF—, —CHF—CH₂— or —CH(CH₃)—CH—.

98. The compound according to claim 78, wherein A represents a group of formula (b): 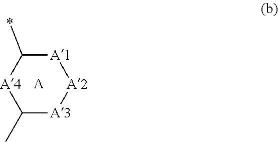
wherein A′1, A′2, A′3, * and A′4 are as defined in claim 78.

99. The compound of formula (I) according to claim 98, wherein A represents 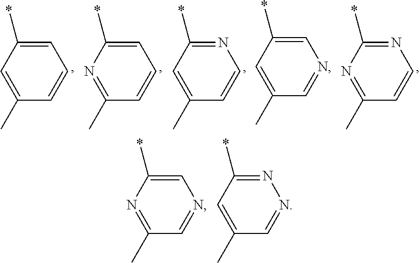
which groups may be unsubstituted or substituted.

100. The compound according to claim 98, wherein A represents a phenyl or a pyridinyl group.

101. The compound according to claim 78, wherein A represents a group of formula (a): 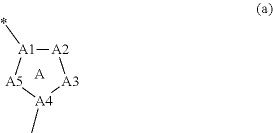
wherein A1, A2, A3, A4, and A5 are as defined in claim 78.

102. The compound according to claim 101, wherein A represents 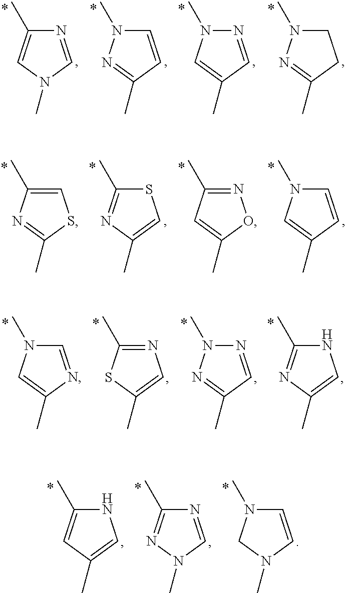
which groups may be unsubstituted or substituted.

103. The compound according to claim 101, wherein A represents a triazolyl or a pyrazolyl group.

104. The compound according to claim 78, which is a compound of formula (I-a): 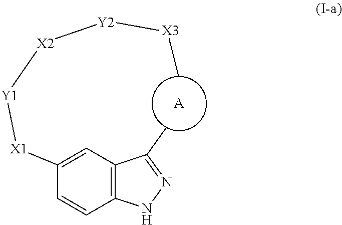
wherein X1, X2, X3, Y1 Y2 and A are as defined in claim 78.

105. The compound according to claim 78, which is a compound of formula (I-b): 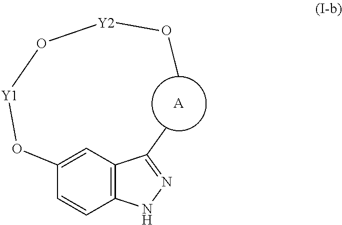
wherein Y1, Y2 and A are as defined in claim 78.

106. The compound according to claim 78, which is a compound of formula (I-ba): 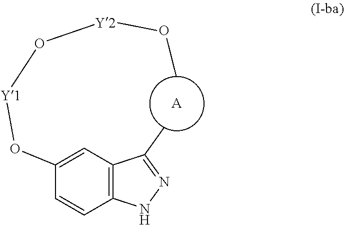
wherein A is as defined in claim 78, and Y′1 and Y′2, which may be identical or different, each represent a linear or branched (C₂-C₆) alkanediyl group which is unsubstituted or substituted with one or more groups selected from halogen atoms and alkoxy groups.

107. The compound according to claim 78, which is a compound of formula (I-b1): 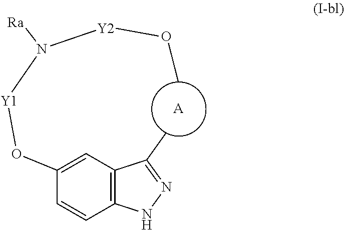
wherein Y1, Y2, Ra and A are as defined in claim 78.

108. The compound according to claim 78, which is a compound of formula (I-b1a): 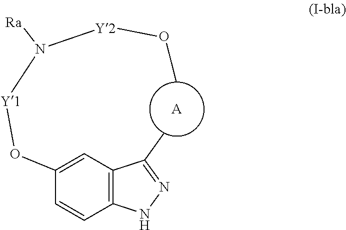
wherein A and Ra are as defined in claim 78, and Y′1 and Y′2, which may be identical or different, each represent a linear or branched (C₂-C₆) alkanediyl group which is unsubstituted or substituted with one or more groups selected from halogen atoms and alkoxy groups.

109. The compound according to claim 78, which is a compound of formula (I-b1b): 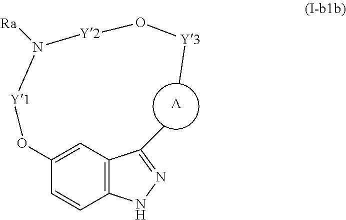
wherein A and Ra are as defined in claim 78, Y′1 and Y′2, which may be identical or different, each represent a linear or branched (C₂-C₆) alkanediyl group which is unsubstituted or substituted with one or more groups selected from halogen atoms and alkoxy groups, and Y′3 represents a linear or branched (C₁-C₆) alkanediyl group which is unsubstituted or substituted with one or more groups selected from halogen atoms and alkoxy groups.

110. The compound according to claim 78, which is a compound of formula (I-b1c): 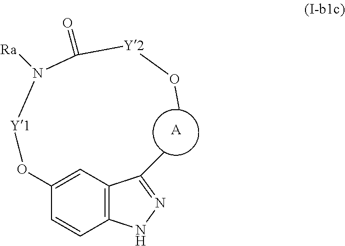
wherein A and Ra are as defined in claim 78, Y′1 represents a linear or branched (C₂-C₆) alkanediyl group which is unsubstituted or substituted with one or more groups selected from halogen atoms and alkoxy groups, and Y″2 represents a linear or branched (C₁-C₅) alkanediyl group which is unsubstituted or substituted with one or more groups selected from halogen atoms and alkoxy groups.

111. The compound according to claim 78, which is a compound of formula (I-b1d): 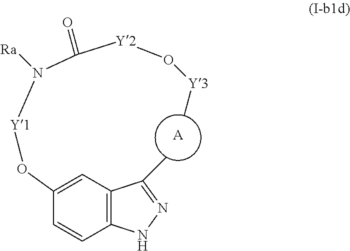
wherein A and R a are as defined in claim 78, Y′1 represents a linear or branched (C₂-C₆) selected from halogen atoms and alkoxy groups, Y″2 represents a linear or branched (C₁-C₅) alkanediyl group which is unsubstituted or substituted with one or more groups selected from halogen atoms and alkoxy groups, and Y′3 represents a linear or branched (C₁-C₆) alkanediyl group which is unsubstituted or substituted with one or more groups selected from halogen atoms and alkoxy groups.

112. The compound according to claim 78, which is a compound of formula (I-c): 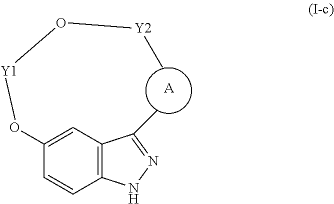
wherein Y1, Y2 and A are as defined in claim 78.

113. The compound according to claim 78, which is a compound of formula (I-ca): 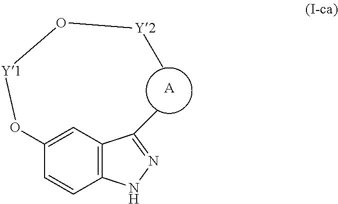
wherein A is as defined in claim 78, and Y′1 and Y′2, which may be identical or different, each represent a linear or branched (C₂-C₆) alkanediyl group which is unsubstituted or substituted with one or more groups selected from halogen atoms and alkoxy groups.

114. The compound according to claim 78, which is a compound of formula (I-c1): 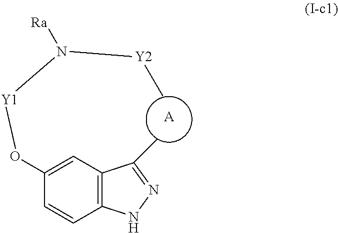
wherein Y1, Y2, Ra and A are as defined in claim 78.

115. The compound according to claim 78, which is a compound of formula (I-c1a): 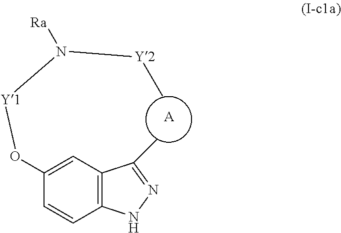
wherein A and Ra are as defined in claim 78, and Y′1 and Y′2, which may be identical or different, each represent a linear or branched (C₂-C₆)alkanediyl group which is, unsubstituted or substituted with one or more groups selected from halogen atoms and alkoxy groups.

116. The compound according to claim 78, which is a compound of formula (I-c1b): 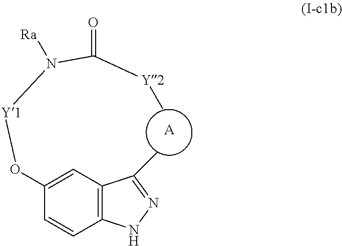
wherein A and Ra are as defined in claim 78, Y′1 represents a linear or branched (C₂-C₆) alkanediyl group which is unsubstituted or substituted with one or more groups selected from halogen atoms and alkoxy groups, and Y″2 represents a linear or branched (C₁-C₅) alkanediyl group which is unsubstituted or substituted with one or more groups selected from halogen atoms and alkoxy groups.

117. The compound according to claim 78, which is compound of formula (I-d): 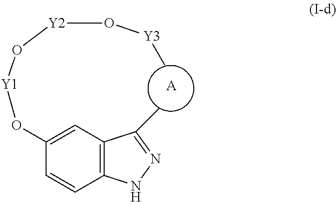
wherein Y1, Y2, Y3 and A are as defined in claim 78.

118. The compound according to claim 78, which is compound of formula (I-da): 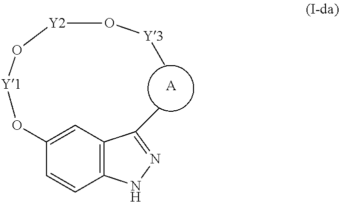
wherein A is as defined in claim 78, Y′1 and Y′2, which may be identical or different, each represent a linear or branched (C₂-C₆) alkanediyl group which is unsubstituted or substituted with one or more groups selected from halogen atoms and alkoxy groups, and Y′3 represents linear or branched (C₁-C₆) alkanediyl group which is unsubstituted or substituted with one or more groups selected from halogen atoms and alkoxy groups.

119. The compound according to claim 78, which is a compound of formula (I-e): 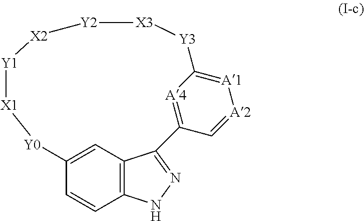
wherein X1, X2, X3, Y0, Y1, Y2, Y3, A′1, A′2 and A′4 are as defined in claim 78.

120. The compound according to claim 78, which is compound of formula (I-f): 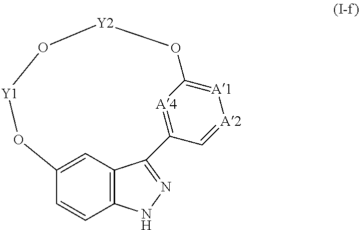
wherein Y1, Y2, A1, A′2 and A′4 are as defined in claim 78.

121. The compound according to claim 78, which is a compound of formula (I-fa): 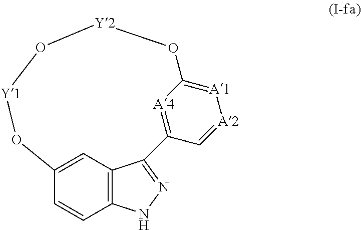
wherein A′ 1, A′2 and A′4 are as defined in claim 78, and Y′1 and Y′2, which may be identical or different, each represent a linear or branched (C₂-C₆) alkanediyl group which is unsubstituted or substituted with one or more groups selected from halogen atoms and alkoxy groups.

122. The compound according to claim 78, which is a compound of formula (I-fb): 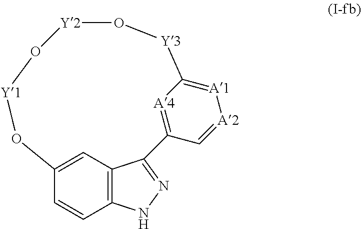
wherein A′1, A′2 and A′4 are as defined in claim 78, and Y′1 and Y′2, which may be identical or different, each represent a linear or branched (C₂-C₆) alkanediyl group which is unsubstituted or substituted with one or more groups selected from halogen atoms and alkoxy groups, and Y′3 represents a linear or branched (C₁-C₆) alkanediyl group which is unsubstituted or substituted with one or more groups selected from halogen atoms and alkoxy groups.

123. The compound according to claim 78, which is a compound of formula (I-f1): 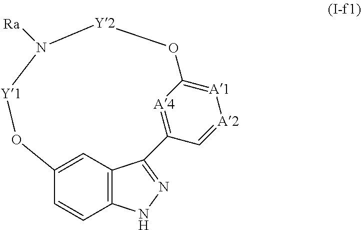
wherein Ra, Y1, Y2, A′1, A′2 and A′4 are as defined in claim 78.

124. The compound according to claim 78, which is a compound of formula (I-f1a): 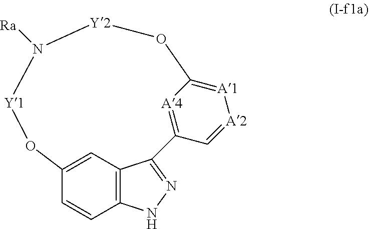
wherein Ra, A′1, A′2 and A′4 are as defined in claim 78, and Y′1 and Y′2, which may be identical or different, each represent a linear or branched (C₂-C₆) alkanediyl group which is unsubstituted or substituted with one or more groups selected from halogen atoms and alkoxy groups.

125. The compound according to claim 78, which is a compound of formula (I-f1b): 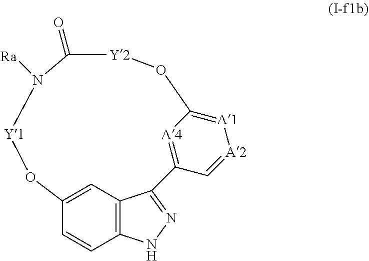
wherein Ra, A′1, A′2 and A′4 are as defined in claim 78, Y′1 represents a linear or branched (C₂-C₆) alkanediyl group which is unsubstituted or substituted with one or more groups selected from halogen atoms and alkoxy groups, and Y″2 represents a linear or branched (C₁-C₅) alkanediyl group which is unsubstituted or substituted with one or more groups selected from halogen atoms and alkoxy groups.

126. The compound according to claim 78, which is compound of formula (I-f1c): 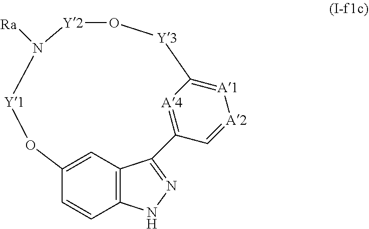
wherein Ra, A′1, A′2 and A′4 are as defined in claim 78, Y′1 and Y′2, which may be identical or different, each represent a linear or branched (C₂-C₆) alkanediyl group which is unsubstituted or substituted with one or more groups selected from halogen atoms and alkoxy groups, and Y′3 represents a linear or branched (C₁-C₆) alkanediyl group which is unsubstituted or substituted with one or more groups selected from halogen atoms and alkoxy groups.

127. The compound according to claim 78, which is a compound of formula (I-f1d): 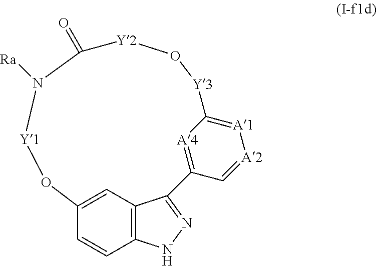
wherein Ra, A′1, A′2 and A′4 are as defined in claim 78, Y′1 represents a linear or branched (C₂-C₆) alkanediyl group which is unsubstituted or substituted with one or more groups selected from halogen atoms and alkoxy groups, Y″2 represents a linear or branched (C₁-C₅) alkanediyl group which is unsubstituted or substituted with one or more groups selected from halogen atoms and alkoxy groups, and Y′3 represents a linear or branched (C₁-C₆) alkanediyl group which is unsubstituted or substituted with one or more groups selected from halogen atoms and alkoxy groups.

128. The compound according to claim 78, which is a compound of formula (I-g): 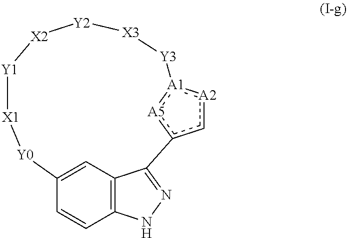
wherein X1, X2, X3, Y0, Y1, Y2, Y3, A1, A2, A5 are as defined in claim 78, and the dotted lines mean that the bond may be single or double.

129. The compound according to claim 78, which is a compound of formula (I-h): 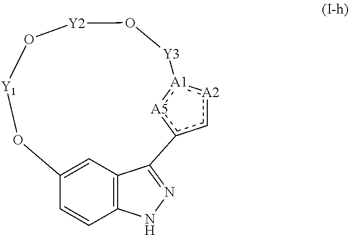
wherein Y1, Y2, Y3, A1, A2, A5 are as defined in claim 78, and the dotted lines mean that the bond may be single or double.

130. The compound according to claim 78, which is compound of formula (I-ha): 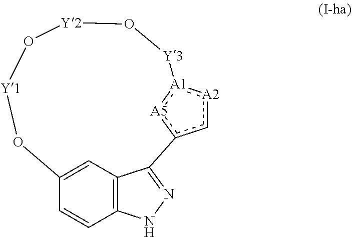
wherein A1, A2, A5 are as defined in claim 78, the dotted lines mean that the bond may be single or double, Y′1 and Y′2, which may be identical or different, each represent a linear or branched (C₂-C₆) alkanediyl group which is unsubstituted or substituted with one or more groups selected from halogen atoms and alkoxy groups, and Y′3 represents a linear or branched (C₁-C₆)alkanediyl group which is unsubstituted or substituted with one or more groups selected from halogen atoms and alkoxy groups.

131. The compound according to claim 78, which is compound of formula (I-h1): 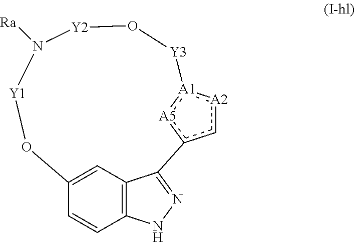
wherein Y1, Y2, Y3, Ra, A1, A2, A5 are as defined in claim 78, and the dotted lines mean that the bond may be single or double.

132. The compound according to claim 78, which is compound of formula (I-h1a): 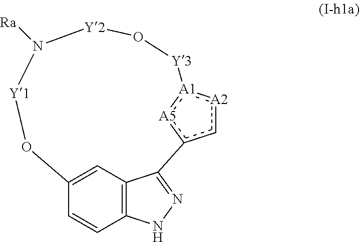
wherein Ra, A1, A2, A5 are as defined in claim 78, the dotted lines mean that the bond may be single or double, Y′1 and Y′2, which may be identical or different, each represent a linear or branched (C₂-C₆) alkanediyl group which is unsubstituted or substituted with one or more groups selected from halogen atoms and alkoxy groups, and Y′3 represents a linear or branched (C₁-C₆) alkanediyl group which is unsubstituted or substituted with one or more groups selected from halogen atoms and alkoxy groups.

133. The compound according to claim 78, which is a compound of formula (I-h1b): 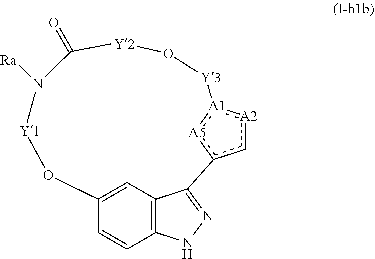
wherein Ra, A1, A2, A5 are as defined in claim 78, the dotted lines mean that the bond may be single or double, Y′1 represents a linear or branched (C₂-C₆) alkanediyl group which is unsubstituted or substituted with one or more groups selected from halogen atoms and alkoxy groups, Y″2 represents a linear or branched (C₁-C₅) alkanediyl group which is unsubstituted or substituted with one or more groups selected from halogen atoms and alkoxy groups, and Y′3 represents a linear or branched (C₁-C₆) alkanediyl group which is unsubstituted or substituted with one or more groups selected from halogen atoms and alkoxy groups.

134. The compound according to claim 78, which is compound of formula (I-i): 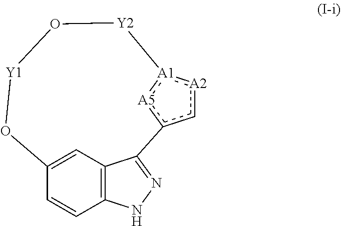
wherein Y1, Y2, A1, A2, A5 are as defined in claim 78, and the dotted lines mean that the bond may be single or double.

135. The compound according to claim 78, which is a compound of formula (I-ia): 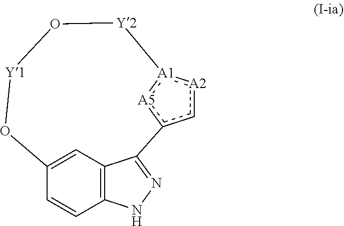
wherein A1, A2, A5 are as defined in claim 78, the dotted lines mean that the bond may be single or double, and Y′1 and Y′2, which may be identical or different, each represent a linear or branched (C₂-C₆) alkanediyl group which is unsubstituted or substituted with one or more groups selected from halogen atoms and alkoxy groups.

136. The compound according to claim 78, which is a compound of formula (I-i1): 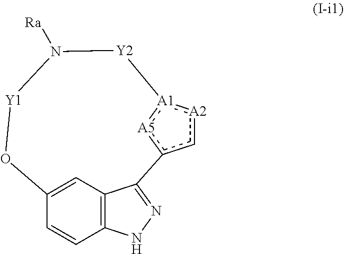
wherein Y1, Y2, Ra, A1, A2, A5 are as defined in claim 78, and the dotted lines mean that the bond may be single or double.

137. The compound according to claim 78, which is a compound of formula (I-i1a): 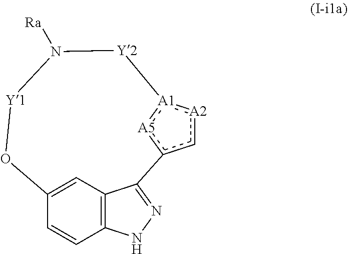
wherein Ra, A1, A2, A5 are as defined in claim 78, the dotted lines mean that the bond may be single or double, and Y′1 and Y′2, which may be identical or different, each represent a linear or branched (C₂-C₆) alkanediyl group which is unsubstituted or substituted with one or more groups selected from halogen atoms and alkoxy groups.

138. The compound according to claim 78, which is a compound of formula (I-i1b): 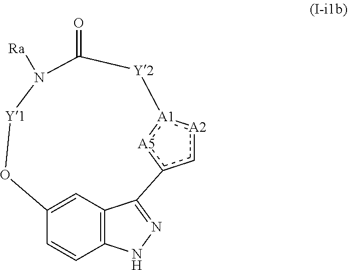
wherein Ra, A1, A2, A5 are as defined in claim 78, (the dotted lines mean that the bond may be single or double, Y′1 represents a linear or branched (C₂-C₆) alkanediyl group which is unsubstituted or substituted with one or more groups selected from halogen atoms and alkoxy groups, and Y″2 represents a linear or branched (C₁-C₅) alkanediyl group which is unsubstituted or substituted with one or more groups selected from halogen atoms and alkoxy groups.

139. The compound according to claim 78, wherein the —Y0-X1-Y1-X2-Y2-X3-Y3- chain represents a group selected from the following, to be read in the same direction from left to right:
—O—(CH₂)₂—O—(CH₂)₂—O—(CH₂)₂—,
—O—CH₂—CH(Me)-O—(CH₂)₂—O—(CH₂)₂—,
—O—CH(Me)-CH₂—O—(CH₂)₂—O—(CH₂)₂—,
—O—(CH₂)₂—O—(CH₂)₂—O—CH(Me)-CH₂—,
—O—(CH₂)₂—O—(CH₂)₂—O—CH₂—CH(Me)-,
—O—CH₂—CH(Me)-O—(CH₂)₂—O—(CH₂)₂—CH(Me)-,
—O—(CH₂)₂—O—CH(Me)-CH₂—O—(CH₂)₂—,
—O—CH(Me)-CH₂—O—(CH₂)₂—O—CH₂—CH(Me)-,
—O—CH(Me)-CH₂—O—(CH₂)₂—O—CH₂—CH(F)—,
—O—CH(Me)-CH(Me)-O—(CH₂)₂—O—(CH₂)₂—,
—O—CF₂—CH₂—O—(CH₂)₂—O—(CH₂)₂—,
—O—(CH₂)₃—O—(CH₂)₃—,
—O—CH₂—CF₂—CH₂—O—(CH₂)₃—,
—O—CH(Me)-(CH₂)₂—O—(CH₂)—,
—O—CH(Me)-CH₂—CH(Me)-O—(CH₂)₃—,
—O—(CH₂)₃—O—CH(Me)-CH₂—CH(Me)-,
—O—CH(Me)-(CH₂)₂—O—(CD₂)₃-,
—O—CH(Me)-(CH₂)₂—O—(CH₂)₂—CH(Me)-,
—O—CH(Me)-(CH₂)₂—O—CH(Me)-(CH₂)₂,
—O—CH(Me)-(CH₂)₂—O—CH₂—CH(Me)-CH₂—,
—O—(CH₂)₃—O—CH(Me)-(CH₂)₂—,
—O—(CH₂)₃—O—CH₂—CH(Me)₂-CH₂—,
—O—(CH₂)₃—O—(CH₂)₂—CH(Me)-,
—O—CH(Me)-CH₂—CH(Me)-O—CH(Me)-(CH₂)₂—,
—O—CH(Me)-CH₂—O—(CH₂)₄—,
—O(CH₂)₃—O—(CH₂)₂—O—,
—O—CH(Me)-(CH₂)₂—O—CH₂—CH(Me)O—,
—O—CH(Me)-(CH₂)₂—O—CH(Me)-CH₂—O—,
—O—CH(Me)-(CH₂)₂—O—(CH₂)₂—O—,
—O—(CH₂)₃—O—CH₂—CH(Me)-O—,
—O—(CH₂)₂—O—(CH₂)₃—O—,
—O—CH(Me)CH₂—O—(CH₂)₃—O—,
—O—CH(Me)-CH₂—O—(CH₂)₂—CH(Me)-O—,
—O—(CH₂)₃—O—(CH₂)₄—O—,
—CH(Me)-(CH₂)₂—O(CH₂)₂—O—CH₂—,
—O—CH(Me)-CH₂—O—(CH₂)₂—O—CH(Me)-,
—O—(CH₂)₂—O—(CH₂)₃—O—CH₂— and
—O—CH(Me)-(CH₂)₂O—(CH₂)₂—O—(CH₂)₂—.

140. The compound according to claim 78, wherein the —Y0-X1-Y1-X2-Y2-X3-Y3- chain represents a group selected from the following, to be read in the same direction from left to right:
—O—(CH₂)₃—NHC(O)—CH₂—O—,
—O—(CH₂)₃—N(Me)C(O)—CH₂—O—,
—O—(CH₂)₂—NHC(O)—CH₂—O—,
—OCH(Me)-(CH₂)₂—NHC(O)—CH₂—O—,
—O—CH₂—CF₂—CH₂—NHC(O)CH₂—O—,
—O—CH₂—(CH(Me))₂—NHC(O)CH₂—O—,
—O—(CH₂)₃—NHC(O)—CH(Me)-O—,
—O—CH₂—CHF—CH(Me)-NHC(O)—CH₂—O—,
—O—(CH₂)₃—NHC(O)—(CH₂)₂—,
—O—CH(Me)-(CH₂)₂—NHC(O)—(CH₂)₂—,
—O—CH₂—CF₂—CH₂—NHC(O)—(CH₂)₂—,
—O—CH₂—CHF—CH₂—NHC(O)—(CH₂)₂— and
—O—CH(Me)-(CH₂)₂—NHC(O)—CH₂—CH(Me)-.

141. The compound according to claim 78, wherein the —Y0-X1-Y1-X2-Y2-X3-Y3- chain represents a group selected from the following, to be read in the same direction from left to right:
—O(CH₂)₃—NH—(CH₂)₂—O—,
—O—(CH₂)₂—NH—(CH₂)₃—O—,
—O—(CH₂)₃—NH—CH₂—CH(Me)-O—,
—O—CH(Me)-(CH₂)₂—NH—(CH₂)₂—O—,
—O—CH(Me)-CH₂—NH—(CH₂)₃—O—,
—O—CH(Me)-CH₂—NH—(CH₂)₂—CH(Me)O—,
—O—CH(Me)-(CH₂)₂—NH—CH₂—CH(Me)-O—,
—O—CH₂—(CH(Me))₂—NH—(CH₂)₂—O—,
—O—CH₂—CHF—CH(Me)-NH—(CH₂)₂—O—,
—O—(CH₂)₂—CH(Me)-NH—(CH₂)₂—O—,
—O—(CH₂)₃—NH—(CH₂)₃—,
—O—CH(Me)-(CH₂)₂—NH—(CH₂)₃—,
—O—CH(Me)-(CH₂)₂—NH—(CH₂)₂—CH(Me)- and
—O—CH(Me)-(CH₂)₂—N(Me)-CH₂—CH(Me)-.

142. The compound according to claim 78, which is selected from:
4-(morpholin-4-yl)-7,10-dioxa-13,18,19-triazatetracyclo[12.5.2.1^{2,6}.0^{17,20}]docosa-1(19), 2(22),3,5,14(21),15,17(20)-heptaene;
7,10,14-trioxa-4,19,20-triazatetracyclo[13.5.2.1^{2,6}.0^{18,21}]tricosa-(20),2(23),3,5,15(22), 16,18(21)-heptaene;
4-(morpholin-4-yl)-7,11,14-trioxa-19,20-diazatetracyclo[13.5.2.1^{2,6}.0^{18,21}]tricosa-1(20), 2(23),3,5,15(22),16,18(21)-heptaene;
7,10-dioxa-4,14,19,20-tetraazatetracyclo[13.5.2.1^{2,6}.0^{18,21}]tricosa-1(20),2(23),3,5,15(22), 16,18(21)-heptaene;
9,13-dioxa-4,5,18,19-tetraazatetracyclo[12.5.2.1^{2,5}.0^{17,20}]docosa-1(19),2(22),3,14(21),15, 17(20)-hexaene;
10,14-dioxa-4,5,19,20-tetraazatetracyclo[13.5.2.1^{2,5}.0^{18,21}]tricosa-1(20),2(23),3,15(22),16, 18(21)-hexaene;
(13R)-13-methyl-7,11,14-trioxa-5,19,20,23-tetraazatetracyclo[13.5.2.1^{2,6}.0^{18,21}]tricosa-1(20),2(23),3,5,15(22),16,18(21)-heptaene;
(6R)-6-methyl-9,13-dioxa-4,5,18,19-tetraazatetracyclo[12.5.2.1^{2,5}.0^{17,20}]docosa-1(19), 2(22),3,14(21),15,17(20)-hexaene;
(7S,13S)-7,13-dimethyl-8,11,14-trioxa-4,19,20-triazatetracyclo[13.5.2.1^{2,6}.0^{18,21}]tricosa-1(20),2(23),3,5,15(22),16,18(21)-heptaene;
(7R,13S)-7,13-demethyl-8,11,14-trioxa-4,19,20-triazatetracyclo[13.5.2.1^{2,6}.0^{18,21}]tricosa-1(20),2(23),3,5,15(22),16,18(21)-heptaene;
(8S,13R)-8,13-dimethyl-7,11,14-trioxa-4,19,20-triazatetracyclo[13.5.2.1^{2,6}.0^{18,21}]tricosa-1(20),2(23),3,5,15(22),16,18(21)-heptaene;
(8S,13R)-8,13-dimethyl-7,11,14-trioxa-4,19,20-triazatetracyclo[13.5.2.1^{2,6}.0^{18,21}]tricosa-1(20),2(23),3,5,15(22),16,18(21)-heptaene;
(7R,13R)-7,13-demethyl-8,11,14-trioxa-4,19,20-triazatetracyclo[13.5.2.1^{2,6}.0^{18,21}]tricosa-1(20),2(23),3,5,15(22),16,18(21)-heptaene hydrochloride;
(7S,13R)-7,13-dimethyl-8,11,14-trioxa-4,19,20-triazatetracyclo[13.5.2.1^{2,6}.0^{18,21}]tricosa-1(20),2(23),3,5,15(22),16,18(21)-heptaene hydochloride;
(6S)-6-methyl-9,13-dioxa-4,5,18,19-tetraazatetracyclo[12.5.2.1^{2,5}.0^{17,20}]docosa-1(19), 2(22),3,14(21),15,17(20)-hexaene;
(13S)-13-methyl-7,10,14-trioxa-5,19,20,23-tetraazatetracyclo[13.5.2.1^{2,6}.0^{18,21}]tricosa-1(20),2(23),3,5,15(22),16,18(21)-heptaene,
(8R,13R)-8,13-dimethyl-7,11,14-trioxa-4,19,20-triazatetracyclo[13.5.2.1^{2,6}.0^{18,21}]tricosa-1(20),2(23),3,5,15(22),16,18(21)-heptaene;
(8R,13S)-8,13-dimethyl-7,11,14-trioxa-4,19,20-triazatetracyclo[13.5.2.1^{2,6}.0^{18,21}]tricosa-1(20),2(23),3,5,15(22),16,18(21)-heptaene;
(13S)-13-methyl-4-(pyrrolidin-1-yl)-7,11,14-trioxa-5,19,20,23-tetraazatetracyclo [13.5.2.1^{2,6}.0^{18,21}]tricosa-1(20),2(23),3,5,15(22),16,18(21)heptaene;
(8S,13S)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene;
(8R,13S)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene;
(8S,13R)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene;
(8R,13R)-8,13-dimethyl-7,10,14-trioxa-4,19,20-triazatetracyclo[13.5.2.1^{2,6}.0^{18,21}]tricosa-1(20),2(23),3,5,15(22),16,18(21)-heptaene;
(13S)-13-methyl-8,11,14-trioxa-4,5,19,20-tetraazatetracyclo[13.5.2.1^{2,5}.0^{18,21}]tricosa-1(20),2(23),3,15(22),16,18(21)-hexaene;
(6R,12S)-6,12-dimethyl-9,13-dioxa-4,5,18,19-tetraazatetracyclo[12.5.2.1^{2,5}.0^{17,20}]docosa-1(19),2(22),3,14(21),15,17(20)-hexaene;
(13S)-13-methyl-7,11,14-trioxa-5,19,20,23-tetraazatetracyclo[13.5.2.1^{2,5}.0^{18,21}]tricosa-1(20),2(23),3,5,15(22),16,18(21)-heptene;
(13S)-4-(3-methoxyazetidin-1-yl)-13-methyl-7,11,14-trioxa-5,19,20,23-tetraazatetracyclo [13.5.2.1^{2,6}.0^{18,21}]tricosa-1(20),2(23),3,5,15(22),16,18(21)heptaene;
(13S)-13-methyl-7,10,14-trioxa-19,20,23-triazatetracyclo[13.5.2.1^{2,6}.0^{18,21}]tricosa-1(20), 2(23),3,5,15(22),16,18(21)-heptaene;
(13S)-4,13-dimethyl-7,11,14-trioxa-5,19,20,23-tetraazatetracyclo[13.5.2.1^{2,6}.0^{18,21}]tricosa-1(20),2(23),3,5,15(22),16,18(21)-heptaene;
(13S)-13-methyl-7,10-dioxa-5,14,19,20,23-pentaazatetracyclo[13.5.2.1^{2,6}.0^{18,21}]tricosa-1(20),2(23),3,5,15(22),16,18(21)-heptaene;
7,7-dimethyl-9,13-dioxa-4,5,18,19-tetraazatetracyclo[12.5.2.1^{2,5}.0^{17,20}]docosa-1(19),2(22), 3,14(21),15,17(20)-hexaene;
(12S)-12-methyl-9,13-dioxa-4,5,18,19-tetraazatetracyclo[12.5.2.1^{2,5}.0^{17,20}]docosa-1(19) 2(22),3,14(21),15,17(20)-hexaene;
(13S)-13-methyl-7,10,14-trioxa-19,20,23-triazatetracyclo[13.5.2.1^{2,6}.0^{18,21}]tricosa-1(20), 2(23),3,5,15(22),16,18(21)-heptaene;
(13S)-4-(3-methoxyazetidin-1-yl)-13-methyl-7,10-dioxa-5,14,19,20,23-pentaazatetracyclo [13.5.2.1^{2,6}.0^{18,21}]tricosa-1(20),2,4,6(23),15(22),16,18(21)-heptaene;
(6R,12R)-6,12-dimethyl-9,13-dioxa-4,5,18,19-tetraazatetracyclo[12.5.2.1^{2,5}.0^{17,20}]docosa-1(19),2(22),3,14(21),15,17(20)-hexaene;
(6S,12R)-6,12-dimethyl-9,13-dioxa-4,3,18,19-tetraazatetracyclo[12.5.2.1^{2,5}.0^{17,20}]docosa-1(19),2(22),3,14(21),15,17(20)-hexaene;
(6S)-6-methyl-9,13-dioxa-4,3,18,19-tetraazatetracyclo[12.5.2.1^{2,5}.0^{17,20}]docosa-1(19), 2(22),3,14(21),15,17(20)-hexaene;
(6S,12S)-6,12-dimethyl-9,13-dioxa-4,5,18,19-tetraazatetracyclo[12.5.2.1^{2,5}.0^{17,20}]docosa-1(19),2(22),3,14(21),15,17(20)-hexaene;
(13S)-13-methyl-4-(pyrrolidin-1-yl)-7,10-dioxa-5,14,19,20,23-pentaazatetracyclo [13.5.2.1^{2,6}.0^{18,21}]tricosa-(20),2(23),33,15(22),16,18(21)-heptaene;
(13S)-4,13-dimethyl-7,10-dioxa-5,14,19,20,23-pentaazatetracyclo[13.5.2.1^{2,6}.0^{18,21}] tricosa-1(20),2(23),3,5,15(22),16,18(21)-heptaene;
(13R)-13-methyl-7,10,14-trioxa-4,19,20,23-tetraazatetracyclo[13.5.2.1^{2,6}.0^{18,21}]tricosa-1(20),2(23),3,5,15(22),16,18(21)-heptaene;
(12R)-12-methyl-9,13-dioxa-4,5,18,19-tetraazatetracyclo[12.5.2.1^{2,5}.0^{17,20}]docosa-1(19), 2(22),3,14(21),15,17(20)-hexaene;
(13S)-13-methyl-10,14-dioxa-19,20-diazatetracyclo[13.5.2.1^{2,6}.0^{18,21}]tricosa-1(20),2(23), 3,5,15(22),16,18(21)-heptane;
(8R,13S)-4-(3-methoxyazetidin-1-yl)-8,13-dimethyl-7,10,14-trioxa-5,19,20,23-tetraaza tetracyclo[13.5.2.1^{2,6}.0^{18,21}]tricosa-1(20),2(23),3,5,15(22),16,18(21)-heptane;
(13R)-13-methyl-7,10,14-trioxa-5,19,20,23-tetraazatetracyclo[13.5.2.1^{2,6}.0^{18,21}]tricosa-1(20),2(23),3,5,15(22),16,18(21)-heptaene;
(8S,13S)-8,13-dimethyl-7,10,14-trioxa-5,19,20,23-tetraazatetracyclo[13.5.2.1^{2,6}.0^{18,21}] tricosa-1(20),2(23),3,5,15(22),16,18(21)-heptaene;
(8S,13S)-8,13-dimethyl-7,10,14-trioxa-4,19,20,23-tetraazatetracyclo[13.5.2.1^{2,6}.0^{18,21}] tricosa-(20),2(23),3,5,15(22),16,18(21)-heptaene;
(12S)-12-methyl-9,13-dioxa-3-thia-18,19,22-triazatetracyclo[12.5.2.1^{2,5}.0^{17,20}]docosa-1(19),2(22),4,14(21),15,17(20)-hexaene;
(8R,13S)-4-[(3R)-3-methoxypyrrolidin-1-yl]-8,13-dimethyl-7,10,14-trioxa-5,19,20,23-tetraazatetracyclo[13.5.2.1^{2,6}.0^{18,21}]tricosa-1(20),2(23),3,5,15(22),16,18(21)-heptaene;
(13R)-13-methyl-8,11,14-trioxa-4,5,19,20-tetraazatetracyclo[13.5.2.1^{2,5}.0^{18,21}]tricosa-1(20),2(23),23,15(22),16,18(21)-hexaene;
(9S,13S)-9,13-dimethyl-7,10,14-trioxa-4,19,20,23-tetraazatetracyclo[13.5.2.1^{2,6}.0^{18,21}] tricosa-1(20),2(23),3,5,13(22),16,18(21)-heptane;
(7R,12S)-7,12-dimethyl-9,13-dioxa-4,5,18,194-tetraazatetracyclo[12.5.2.1^{2,5}.0^{17,20}]docosa-1(19),2(22),3,14(21),15,17(20)-hexaene;
(8R,13S)-8,13-dimethyl-7,11,14-trioxa-5,19,20,23-tetraazatetracyclo[13.5.2.1^{2,6}.0^{18,21}] tricosa-1(20),2(23),3,5,15(22),16,18(21)-heptene;
(12S)-12-methyl-8,11,14-trioxa-4,5,19,20-tetraazatetracyclo[13.5.2.1^{2,5}.0^{18,21}]tricosa-1(20),2(23),3,15(22),16,18(21)-hexaene;
(8R)-8-methyl-7,10,14-trioxa-5,19,20,23-tetraazatetracyclo[13.5.2.1^{2,6}.0^{18,21}]tricosa-1(20), 2(23),3,5,13(22),16,18(21)heptaene;
(13R)-13-methyl-7,10,14-trioxa-19,20,23-triazatetracyclo[13.5.2.1^{2,6}.0^{18,21}]tricosa-1(20), 2(23),3,5,15(22),16,18(21)-heptaene;
(12S)-12-methyl-9,13-dioxa-3,5,18,19-tetraazatetracyclo[12.5.2.1^{2,5}.0^{17,20}]docosa-1(19), 2(22),3,14(21),15,17(20)-hexaene;
(9R,13S)-9,13-dimethyl-7,10,14-trioxa-4,19,20,23-triazatetracyclo[13.5.2.1^{2,6}.0^{18,21}] tricosa-1(20),2(23),3,5,15(22),16,18(21)-heptaene;
(9S,13S)-9,13-dimethyl-7,10,14-trioxa-5,19,20,23-tetraazatetracyclo[13.5.2.1^{2,6}.0^{18,21}] tricosa-1(20),2(23),3,5,15(22),16,18(21)-heptaene;
(9R)-9-methyl-8,11,14-trioxa-4,5,19,20-tetraazatetracyclo[13.5.2.1^{2,5}.0^{18,21}]tricosa-1(20), 2(23),3,15(22),16,18(21)-hexaene;
(9S)-9-methyl-8,11,14-trioxa-4,5,19,20-tetraazatetracyclo[13.5.2.1^{2,5}.0^{18,21}]tricosa-1(20), 2(23),3,15(22),16,18(21)-hexaene;
(13S)-13-methyl-7,10,14-trioxa-4-thia-19,20,23-triazatetracyclo[13.5.2.1^{2,5}.0^{18,21}]tricosa-1(20),2,5(23),15(22),16,18(21)-hexaene;
8,11,14-trioxa-4,5,19,20-tetraazatetracyclo[13.5.2.1^{2,5}.0^{18,21}]tricosa-1(20),2(23),3,15(22), 16,18(21)-hexaene;
(6S)-6-methyl-8,11,14-trioxa-4,5,19,20-tetraazatetracyclo[13.5.2.^{12,5}.0^{18,21}]tricosa-1(20), 2(23),3,15(22),16,18(21)-hexaene;
(6R)-6-methyl-8,11,14-trioxa-4,5,19,20-triazatetracyclo[13.5.2.1^{2,5}.0^{18,21}]tricosa-1(20), 2(23),3,15(22),16,18(21)-hexaene;
(13S)-13-methyl-7,10,14-trioxa-23-thia-4,19,20-triazatetracyclo[13.5.2.1^{2,5}.0^{18,21}]tricosa-1(20),2,4,15(22),16,18(21)-hexaene;
(12R)-12-methyl-8,11,14-trioxa-4,5,19,20-tetraazatetracyclo[13.5.2.1^{2,5}.0^{18,21}]tricosa-1(20),2(23),3,15(22),16,18(21)-hexaene;
9,12,15-trioxa-4,20,21-triazatetracyclo[14.5.2.1^{2,6}.0^{19,22}]tetracosa-1(21),2(24),3,5,16(23), 17,19(22)-heptaene;
(14S)-14-methyl-9,12,15-trioxa-4,20,21-triazatetracyclo[14.5.2.1^{2,6}.0^{19,22}]tricosa-1(21), 2(24),3,5,16(23),17,19(22)-heptaene;
(10R)-10-methyl-8,11,14-trioxa-4,5,19,20-tetraazatetracyclo[13.5.2.1^{2,5}.0^{18,21}]tricosa-1(20),2(23),3,15(22),16,18(21)-hexaene,
(13R)-13-methyl-8,11,14-trioxa-4,5,19,20,23-pentaazatetracyclo[13.5.2.1^{2,5}.0^{18,21}]tricosa-1(20),2(23),3,15(22),16,18(21)-hexaene;
(13S)-13-methyl-8,11,14-trioxa-5,19,20-triazatetracyclo[13.5.2.1^{2,5}.0^{18,21}]tricosa-1(20),2(23),3,15(22),16,18(21)-hexaene-4-carbonitrile;
8,11,14-trioxa-4,5,19,20,22-pentaazatetracyclo[13.5.2.1^{2,5}.0^{18,21}]tricosa-1(20),2(23),3,15(22),16,18(21)-hexaene;
9,13-dioxa-4,5,18,19,22-pentaazatetracyclo[12.5.2.1^{2,5}.0^{17,20}]docosa-1(19),2(22),3,14(21), 15,17(20)-hexaene;
(13R)-13-methyl-7,10,14-trioxa-23-thia-4,19,20-triazatetracyclo[13.5.2.1^{2,5}.0^{18,21}]tricosa-1(20),2,4,15(22),16,18(21)-hexaene;
(13R)-4,13-dimethyl-8,11,14-trioxa-4,19,20,23-tetraazatetracyclo[13.5.2.1^{2,5}.0^{18,21}]tricosa-1(20),2,5(23),15(22),16,18(21)-hexaene;
(7R)-7-methyl-8,11,14-trioxa-4,5,19,20-tetraazatetracyclo[13.5.2.1^{2,5}.0^{18,21}]tricosa-1(20), 2(23),3,15(22),16,18(21)-hexaene;
(13,13-difluoro-8,11,14-trioxa-4,5,19,20-tetraazatetracyclo[13.5.2.1^{2,5}.0^{18,21}]tricosa-1(20), 2(23),3,15(22),16,18(21)-hexaene;
(13S)-4,13-dimethyl-7,10,14-trioxa-4,19,20,23-tetraazatetracyclo[13.5.2.1^{2,5}.0^{18,21}]tricosa-1(20),2,5(23),15(22),16,18(21)-hexaene;
(13S)-13-methyl-8,11,14-trioxa-5,19,20,23-tetraazatetracyclo[13.5.2.1^{2,5}.0^{18,21}]tricosa-1(20),2(23),3,15(22),16,18(21)-hexaene;
(13S)-13-methyl-8,11,14-trioxa-4,5,19,20,23-pentaazatetracyclo[13.5.2.1^{2,5}.0^{18,21}]tricosa-1(20),2(23),3,15(22),16,18(21)-hexaene;
(13R)-13-methyl-8,11,14-trioxa-5,19,20-triazatetracyclo[13.5.2.1^{2,5}.0^{18,21}]tricosa-1(20), 2(23),3,15(22),16,18(21)-hexaene-4-carbonitrile;
(13R)-13-methyl-8,11,14-trioxa-5,19,20,23-tetraazatetracyclo[13.5.2.1^{2,5}.0^{18,21}]tricosa-1(20),2(23),3,15(22),16,18(21)-hexaene;
(13S)-4,13-dimethyl-7,10,14-trioxa-4,19,20,23-tetraazatetracyclo[13.5.2.1^{2,5}.0^{18,21}]tricosa-1(20),2,5(23),15(22),16,18(21)-hexaone;
(12S)-12-methyl-8,11,14-trioxa-5,19,20-triazatetracyclo[13.5.2.1^{2,5}.0^{18,21}]tricosa-1(20), 2(23),3,15(22),16,18(21)-hexaene-4-carbonitrile;
(12R)-12-methyl-8,11,14-trioxa-5,19,20-triazatetracyclo[13.5.2.1^{2,5}.0^{18,21}]tricosa-1(20), 2(23),3,15(22),16,18(21)-hexaene-4-carbonitrile;
(6R,12S)-6,12-dimethyl-8,11,14-trioxa-4,5,19,20-tetraazatetracyclo[13.5.2.1^{2,5}.0^{18,21}] tricosa-1(20),2(23),3,15(22),16,18(21)-hexaene;
(13R)-17-fluoro-13-methyl-8,11,14-trioxa-4,5,19,20-tetraazatetracyclo[13.5.2.1^{2,5}.0^{18,21}] tricosa-1(20),2(23),3,15(22),16,18(21)-hexaene;
(6S,12S)-6,12-dimethyl-8,11,14-trioxa-4,5,19,20-tetraazatetracyclo[13.5.2.1^{2,5}.0^{18,21}] tricosa-1(20),2(23),3,15(22),16,18(21)-hexaene;
(12R)-12-methyl-9,13-dioxa-4,5,18,19,22-pentaazatetracyclo[12.5.2.1^{2,5}.0^{17,20}]docosa-1(19),2(22),3,14(21),15,17(20)-hexaene;
13-methyl-4-(morpholin-4-yl)-7,10-dioxa-13,19,20-triazatetracyclo[13.5.2.1^{2,5}.0^{18,21}] tricosa-1(20),2,4,6(23),15,17,21-heptaene;
(12S)-12-methyl-9,13-dioxa-4,5,18,19,22-pentaazatetracyclo[12.5.2.1^{2,5}.0^{17,20}]docosa-1(19),2(22),3,14(21),1S,17(20)-hexaene;
(6S,13R)-6,13-dimethyl-8,11,14-trioxa-4,5,19,20-tetraazatetracyclo[13.5.2.1^{2,5}.0^{18,21}] tricosa-1(20),2(23),3,15(22),16,18(21)-hexaene;
(6R,12R)-6,12-dimethyl-9,13-dioxa-4,5,18,19,22-pentaazatetracyclo[12.5.2.1^{2,5}.0^{17,20}] docosa-1(19),2(22),3,14(21),15,17(20)-hexaene;
(13R)-6-methoxy-13-methyl-8,11,14-trioxa-4,5,19,20-tetraazatetracyclo[13.5.2.1^{2,5}.0^{18,21}] tricosa-(20),2(23),3,15(22),16,18(21)-hexaene;
8,11,14-trioxa-4,5,16,19,20-pentaazatetracyclo[13.5.2.1^{2,5}.0^{18,21}]tricosa-(20),2(23),3, 15(22),16,18(21)-hexaene;
(8S,12R)-8,12-dimethyl-9,13-dioxa-4,5,18,19-tetraaza tetracyclo[12.5.2.1^{2,5}.0^{17,20}]docosa-1(19),2(22),3,14(21),15,17(20)hexaene;
(8R,12R)-8,12-dimethyl-9,13-dioxo-4,5,18,19-tetraazatetracyclo[12.5.2.1^{2,5}.0^{17,20}]docosa-1(19),2(22),3,14(21),15,17(20)-hexaene;
(12S,13R)-12,13-dimethyl-8,11,14-trioxa-4,5,19,2-tetraazatetracyclo[13.5.2.1^{2,5}.0^{18,21}] tricosa-1(20),2(23),3,15(22),16,18(21)-hexaene;
(12R,13S)-12,13-dimethyl-8,11,14-trioxa-4,5,19,20-tetraazatetracyclo[13.5.2.1^{2,5}.0^{18,21}] tricosa-1(20),2(23),3,15(22),16,18(21)-hexaene;
(6S,13S)-6,13-dimethyl-8,11,14-trioxa-4,5,19,20-tetraazatetracyclo[13.5.2.1^{2,5}.0^{18,21}] tricosa-1(20),2(23),3,15(22),16,18(21)-hexaene;
8,13-dioxa-4,5,18,19-tetraazatetracyclo[12.5.2.1^{2,5}.0^{17,20}]docosa-1(19),2(22),3,14(21),15, 17(20)-hexaen;
(6S,12R)-6,12-dimethyl-9,13-dioxa-4,5,18,19,22-pentaazatetracyclo[12.5.2.1^{2,5}.0^{17,20}] docosa-1(19),2(22),3,14(21),15,17(20)-hexaene;
10,13-dioxa-4,5,18,19-tetraazatetracyclo[12.5.2.1^{2,5}.0^{17,20}]docosa-1(19),2(22),3,14(21), 15,17(20)hexaene;
(6R or 6S,13R)-6-fluoro-13-methyl-8,11,14-trioxa-4,5,19,20-tetraazatetracyclo[13.5.2.1^{2,5}.0^{18,21}]tricosa-1(20),2(23),3,15(22),16,18(21)-hexaene;
(6S or 6R,13R)-6-fluoro-13-methyl-8,11,4-trioxa-4,5,19,20-tetraazatetracyclo[13.5.2.1^{2,5}.0^{18,21}] tricosa-1(20),2(23),3,15(22),16,18(21)-hexaene;
(12S)-12-methyl(9,9,10,10-²H₄)-8,11,14-trioxa-4,5,19,20-tetraazatetracyclo[13.5.2.1^{2,5}.0^{18,21}] tricosa-1(20),2(23),3,15,17,21-hexaene;
8,14-dioxa-4,5,19,20-tetraazatetracyclo[13.5.2.1^{2,5}.0^{18,21}]tricosa-1(20),2(23),3,15(22), 16,18(21)-hexaene;
(6R)-11,11-difluoro-6-methyl-9,13-dioxa-4,5,18,19-tetraazatetracyclo[12.5.2.1^{2,5}.0^{17,20}] docosa-1(19),2(22),3,14(21),15,17(20)-hexaene;
(12S)-12-methyl(6,6,7,7-2H4)-8,11,14-trioxa-4,5,19,20-tetraazatetracyclo[13.5.2.1^{2,5}.0^{18,21}]tricosa-1(20),2(23),3,15(22),16,18(21)-hexaene;
11,11-difluoro-9,13-dioxa-4,5,18,19-tetraazatetracyclo[12.5.2.1^{2,5}.0^{17,20}]docosa-1(19),2(22),3,14(21),15,17(20)-hexaene;
(12S)-12-methyl-11,14-dioxa-4,5,19,20-tetraazatetracyclo[13.5.2.1^{2,5}.0^{18,21}]tricosa-1(20),2(23),3,15(22),16,18(21)-hexaene;
(12R,13R)-12,13-dimethyl-8,11,14-trioxa-4,5,19,20-tetraazatetracyclo[13.5.2.1^{2,5}.0^{18,21}] tricosa-1(20),2(23),3,15(22),16,18(21)-hexaene;
(12S,13S)-12,13-dimethyl-8,11,14-trioxa-4,5,19,20-tetraazatetracyclo[13.5.2.1^{2,5}.0^{18,21}] tricosa-1(20),2(23),3,15(22),16,18(21)-hexaene;
8,13-dioxa-4,5,18,19,22-pentaazatetracyclo[12.5.2.1^{2,5}.0^{17,20}]docosa-1(19),2(22),3,14(21),15,17(20)-hexaene;
(6R,10S)-6,10-dimethyl-9,13-dioxa-4,5,18,19-tetraazatetracyclo[12.5.2.1^{2,5}.0^{17,20}]docosa-1(19),2(22),3,14(21),15,17(20)-hexaene;
(6R,8R)-6,8-dimethyl-9,13-dioxa-4,5,18,19-tetraazatetracyclo[12.5.2.1^{2,5}.0^{17,20}]docosa-1(19),2(22),3,14(21),15,17(20)-hexaene;
7,7-difluoro-9,13-dioxa-4,5,18,19-tetraazatetracyclo[12.5.2.1^{2,5}.0^{17,20}]docosa-1(19),2(22),3,14(21),15,17(20)-hexaene;
(13S)-4,13-dimethyl-7,11,14-trioxa-4,19,20,23-tetraazatetracyclo[13.5.2.1^{2,5}.0^{18,21}]tricosa-1(20),62,5(23),15(22),16,18(21)-hexaene;
(12R)-7,7-difluoro-12-methyl-9,13-dioxa-4,5,18,19-tetraazatetracyclo[12.5.2.1^{2,5}.0^{17,20}] docosa-1(19),2(22),3,14(21),15,17(20)-hexaene;
(8E)-11,14-dioxa-4,5,19,20-tetraazatetracyclo[13.5.2.1^{2,5}.0^{18,21}]tricosa-1(20),2(23),3,8,15(22),16,18(21)-heptaene;
(12S)-12-methyl-8,13-dioxa-4,5,18,19,22-pentaazatetracyclo[12.5.2.1^{2,5}.0^{17,20}]docosa-1(19),2(22),3,14(21),5,17(20)-hexaene;
(6R,10R)-6,10-dimethyl-9,13-dioxa-4,5,18,19-tetraazatetracyclo[12.5.2.1^{2,5}.0^{17,20}]docosa-1(19),2(22),3,14(21),15,17(20)-hexaene;
(12R)-12-methyl-8,13-dioxa-4,5,18,19,22-pentaazatetracyclo[12.5.2.1^{2,5}.0^{17,20}]docosa-1(19),2(22),3,14(21),15,17(20)-hexaene;
(12R)-12-methyl-9,13-dioxa-2,4,18,19,22-pentaazatetracyclo[12.5.2.1^{2,5}.0^{17,20}]docosa-1(19),3,5(22),14(21),15,17(20)-hexaene;
(10R,12R)-10,12-dimethyl-9,13-dioxa-4,5,18,19-tetraazatetracyclo[12.5.2.1^{2,5}.0^{17,20}]docosa-1(19),2(22),3,14(21),15,17(20)-hexaene;
(8R,12R)-8,12-dimethyl-9,13-dioxa-4,5,18,19,22-pentaazatetracyclo[12.5.2.1^{2,5}.0^{17,20}]docosa-1(19),2(22),3,14(21),15,17(20)-hexaene;
(6R,12R)-6,12-dimethyl-8,13-dioxa-4,5,18,19-tetraazatetracyclo[12.5.2.1^{2,5}.0^{17,20}]docosa-1(19),2(22),3,14(21),15,17(20)-hexaene;
(13S)-13-methyl-9,12,15-trioxa-4,5,20,21-tetraazatetracyclo[14.5.2.1^{2,5}.0^{19,22}]tetracosa-1(21),2(24),3,16(23),17,19(22)-hexaene;
(12R)-12-methyl-10,13-dioxa-4,5,18,19,22-pentaazatetracyclo[12.5.2.1^{2,5}.0^{17,20}]docosa-1(19),2(22),3,14(21),15,17(20)-hexaene;
(14R)-14-methyl-8,11,15-trioxa-4,5,20,21-tetraazatetracyclo[14.5.2.1^{2,5}.0^{19,22}]tetracosa-1(21),2(24),3,16(23),17,19(22)-hexaene;
(6R,8S)-6,8-dimethyl-9,13-dioxa-4,5,18,19-tetraazatetracyclo[12.5.2.1^{2,5}.0^{17,20}]docosa-1(19),2(22),3,14(21),15,17(20)-hexaene;
(14R)-14-methyl-8,11,15-trioxa-4,5,20,21,24-pentaazatetracyclo[14.5.2.1^{2,5}.0^{19,22}]tetracosa-1(21),2(24),3,16(23),17,19(22)-hexaene;
7,14-dioxa-10,19,20-triazatetracyclo[13.5.2.1^{2,6}.0^{18,21}]tricosa-1(20),2(23),3,5,15(22), 16,18(21)heptaen-9-one;
7,14-dioxa-10,19,20-triazatetracyclo[13.5.2.1^{2,6}.0^{18,21}]tricosa-1(20),2(23),3,5,15(22) 16,18(21)-heptaene;
7,14-dioxa-4,10,19,20-tetraazatetracyclo[13.5.2.1^{2,6}.0^{18,21}]tricosa-1(20),2,4,6(23),15, 17,21-heptaen-9-one;
7,14-dioxa-4,10,19,20-tetraazatetracyclo[13.5.2.1^{2,6}.0^{18,21}]tricosa-1(20),2,4,6(23),15, 17,21-heptaene;
5-fluoro-7,14-dioxa-10,19,20-triazatetracyclo[13.5.2.1^{2,6}.0^{18,21}]tricosa-(20),2,4,6(23), 15,17,21-heptaen-9-one;
5-fluoro-7,14-dioxa-10,19,20-triazatetracyclo[13.5.2.1^{2,6}.0^{18,21}]tricosa-1(20),2,4,6(23), 15,17,21-heptaene;
5-methyl-7,14-dioxa-10,19,20-triazatetracyclo[13.5.2.1^{2,6}.0^{18,21}]tricosa-1(20),2,4,6(23),15, 17,21-heptaen-9-one;
5-methyl-7,14-diox-10,19,20-triazatetracyclo[13.5.2.1^{2,6}.0^{18,21}]tricosa-1(20),2,4,6(23),15, 17,21-heptaene;
4-chloro-7,14-dioxa-10,19,20-triazatetracyclo[13.5.2.1^{2,6}.0^{18,21}]tricosa-(20),2,4,6(23),15, 17,21-heptaen-9-one;
4-chloro-7,14-dioxa-10,19,20-triazatetracyclo[13.5.2.1^{2,6}.0^{18,21}]tricosa-1(20),2,4,6(23),15, 17,21-heptaene;
4-methyl-7,14-dioxa-10,19,20-triazatetracyclo[13.5.2.1^{2,6}.0^{18,21}]tricosa-1(20),2(23),3,5,13, 17,21-heptaen-9-one;
4-methyl-7,14-dioxa-10,19,20-triazatetracyclo[13.5.2.1^{2,6}.0^{18,21}]tricosa-1(20),2(23),3,15, 17,21-heptaene;
4-(oxan-4-yl)-7,14-dioxa-10,19,20-triazatetracyclo[13.5.2.1^{2,5}.0^{18,21}]tricosa-1(20),2(23), 3,5,15,17,21-heptan-9-one;
4-(oxan-4-yl)-7,14-dioxa-10,19,20-triazatetracyclo[13.5.2.1^{2,6}.0^{18,21}]tricosa-1(20),2(23), 3,5,15,17,21-heptaene;
4-(oxan-3-yl)-7,14-dioxa-10,19,20-triazatetracyclo[13.5.2.1^{2,6}.0^{18,21}]tricosa-1(20),2(23), 3,5,15,17,21-haptaen-9-one;
4-(oxan-3-yl)-7,14-dioxa-10,19,20-triazatetracyclo[13.5.2.1^{2,6}.0^{18,21}]tricosa-1(20),2(23), 3,5,15,17,21-heptaene;
(11S) or (11R)-11-(methoxymethyl)-7,14-dioxa-10,19,20-triazatetracyclo[13.5.2.1^{2,6}.0^{18,21}]tricosa-1(20),2(23),3,5,15,17,21-heptaen-9-on;
(11S) or (11R)-1-(methoxymethyl)-7,14-dioxa-10,19,20-triazatetracyclo[13.5.2.1^{2,6}.0^{18,21}]tricosa-1(20),2(23),3,5,15,17,21-heptene;
(11R) or (11S)-11-(methoxymethyl)-7,14-dioxa-10,19,20-triazatetracyclo[13.5.2.1^{2,6}.0^{18,21}]tricosa-1(20),2(23),3,15,17,21-heptaen-9-one;
(11R) or (11S)-11-(methoxymethyl)-7,14-dioxa-10,19,20-triazatetracyclo[13.5.2.1^{2,6}.0^{18,21}]tricosa-1(20),2(23),3,5,15,17,21-heptane;
11-(fluoromethyl)-7,14-dioxa-10,19,20-triazatetracyclo[13.5.2.1^{2,6}.0^{18,21}]tricosa-1(20),2,4,6(23),15,17,21-heptaen-9-one;
11-(fluoromethyl)-7,14-dioxa-10,19,20-triazatetracyclo[13.5.2.1^{2,6}.0^{18,21}]tricosa-1(20),2,4,6(23),15,17,21-heptaene;
7′,14′-dioxa-4′,10′,19′,20′-tetraazaspiro[cyclopropane-1,8′-tetracyclo[13.5.2.1^{2,6}.0^{18,21}] tricosane]-1′(20′),2′(23′),3′,5′,15′,17,21′-heptaen-9′-one;
7,14′-dioxa-4′,10′,19′,20′-tetraazaspiro[cyclopropane-1,8′-tetracyclo[13.5.2.1^{2,6}.0^{18,21}] tricosane]-1′(20′),2′(23′),3′,5,15′,17,21′-heptaene;
13-methyl-7,14-dioxa-4,10,19,20-tetraazatetracyclo[13.5.2.1^{2,6}.0^{18,21}]tricosa-1(20),2,4, 6(23),15,17,21-heptaen-9-one;
13-methyl-7,14-dioxa-4,10,19,20-tetraazatetracyclo[13.5.2.1^{2,6}.0^{18,21}]tricosa-1(20),2,4, 6(23),15,17,21-heptaene,
8,10-dimethyl-7,14-dioxa-4,10,19,2-tetraazatetracyclo[13.5.2.1^{2,6}.0^{18,21}]tricosa-1(20),2,4, 6(23),15,17,21-heptaen-9-one;
8,10-dimethyl-7,14-dioxa-4,10,19,20-tetraazatetracyclo[13.5.2.1^{2,6}.0^{18,21}]tricosa-1(20),2,4, 6(23),15,17,21-heptaene;
(13R)-13-methyl-7,14-dioxa-4,10,19,20-tetraazatetracyclo[13.5.2.1^{2,6}.0^{18,21}]tricosa-1(20),24,6(23),15(22),16,18(21)-heptaen-9-one;
(13R)-13-methyl-7,14-dioxa-4,10,19,20-tetraazatetracyclo[13.5.2.1^{2,6}.0^{18,21}]tricosa-1(20),2,4,6(23),15(22),16,18(21)-heptaene;
(7S,9S)-15-oxa-11,20,21-triazatetracyclo[14.5.2.1^{2,6}.0^{7,9}.0^{19,22}]tetracosa-1(21),2(24),3, 5,16(23),17,19(22)-heptaen-10-one;
(7S,9S)-15-oxa-11,20,21-triazapentacyclo[14.5.2.1^{2,6}.0^{7,9}.0^{19,22}]tetracosa-1(21),2(24),3,5, 16(23),17,19(22)-heptaene;
14-oxa-10,19,20-triazatetracyclo[13.5.2.1^{2,6}.0^{18,21}]tricosa-1(20),2(23),3,5,15(22),16, 18(21)-heptaen-9-one;
14-oxa-10,19,20-triazatetracyclo[13.5.2.1^{2,6}.0^{18,21}]tricosa-1(20),2(23),3,5,15(22),16, 18(21)-heptaene;
(11S,12S)-11,12-dimethyl-7,14-dioxa-10,19,20-triazatetracyclo[13.5.2.1^{2,6}.0^{18,21}]tricosa-1(20),2(23),3,5,15(22),16,18(21)-heptaen-9-one;
(11S,12S)-11,12-dimethyl-7,14-dioxa-10,19,20-triazatetracyclo[13.5.2.1^{2,6}.0^{18,21}]tricosa-1(20),2(23),3,5,15(22),16,18(21)-heptaene;
12-methoxy-7,14-dioxa-10,19,20-triazatetracyclo[13.5.2.1^{2,6}.0^{18,21}]tricosa-1(20),2(23),3,5, 15,17,21-heptaen-9-one;
12-methoxy-7,14-dioxa-10,19,20-triazatetracyclo[13.5.2.1^{2,6}.0^{18,21}]tricosa-1(20),2(23),3,5, 15,17,21-heptaene;
(13R)-13-methyl-4-(morpholin-4-yl)-7,14-dioxa-10,19,20-triazatetracyclo[13.5.2.1^{2,6}.0^{18,21}]tricosa-1(20),2(23),3,5,15,17,21-heptaen-9-one;
(13R)-13-methyl-4-(morpholin-4-yl)-7,14-dioxa-10,19,20-triazatetracyclo[13.5.2.1^{2,6}.0^{18,21}]tricosa-1(20),2(23),3,5,15,17,21-heptaene;
(13S)-13-methyl-4-(morpholin-4-yl)-7,14-dioxa-10,19,20-triazatetracyclo[13.5.2.1^{2,6}.0^{18,21}]tricosa-1(20),2(23),3,5,15,17,21-heptaen-9-one;
(13S)-13-methyl-4-(morpholin-4-yl)-7,14-dioxa-10,19,20-triazatetracyclo[13.5.2.1^{2,6}.0^{18,21}]tricosa-1(20),2(23),3,5,15,17,21-heptaene;
8-methyl-4-(morpholin-4-yl)-7,14-dioxa-10,19,20-triazatetracyclo[13.5.2.1^{2,6}.0^{18,21}] tricosa-1(20),2,4,6(23),15,17,21-heptaen-9-one;
(8R) or (8S)-8-methy-4-(morpholin-4-yl)-7,14-dioxa-10,19,20-triazatetracyclo[13.5.2.1^{2,6}.0^{18,21}]tricosa-1(20),2,4,6(23),15,17,214-heptaene;
(8S) or (8R)-8-methyl-4-(morpholin-4-yl)-7,14-dioxa-10,19,20-triazatetracyclo[13.5.2.1^{2,6}.0^{18,21}]tricosa-1(20),2,4,6(23),15,17,21-heptaene;
(12R)-12-methyl-13-oxa-4,5,9,18,19-pentaazatetracyclo[12.5.2.1^{2,5}.0^{17,20}]docosa-1(19), 2(22),3,14(21),15,17(20)-hexaen-8-one;
(12R)-12-methyl-13-oxa-4,5,9,18,19-pentaazatetracyclo[12.5.2.1^{2,5}.0^{17,20}]docosa-1(19),2(22),3,14(21),15,17(20)-hexaene;
13-oxa-4,5,9,18,19,22-hexaazatetracyclo[12.5.2.1^{2,5}.0^{17,20}]docosa-1(19),2(22),3,14(21),15, 17(20)-hexaen-4-one;
13-oxa-4,5,9,18,19,22-hexaazatetracyclo[12.5.2.1^{2,5}.0^{17,20}]docosa-1(19),2(22),3,14(21),15, 17(20)-hexaene;
(11S,12S)-12-fluoro-11-methyl-7,14-dioxa-10,19,20-triazatetracyclo[13.5.2.1^{2,6}.0^{18,21}] tricosa-1(20),2,4,6(23),15,17,21-heptaen-9-one;
(11S,12S)-12-fluoro-11-methyl-7,14-dioxa-10,19,20-triazatetracyclo[13.5.2.1^{2,6}.0^{18,21}] tricosa-1(20),2,4,6(23),15,17,21-heptaene;
4-(trifluoromethyl)-7,14-dioxa-10,19,20-triazatetracyclo[13.5.2.1^{2,6}.0^{18,21}]tricosa-1(20),2,4,6(23),15,17,21-heptaen-9-one;
4-(trifluoromethyl)-7,14-dioxa-10,19,20-triazatetracyclo[13.5.2.1^{2,6}.0^{18,21}]tricosa-1(20),2,4,6(23),15,17,21-heptaene;
16-methyl-7,14-dioxa-4,10,19,20-tetraazatetracyclo[13.5.2.1^{2,6}.0^{18,21}]tricosa-1(20),2(23), 3,5,15(22),16,18(21)-heptaen-9-one;
16-methyl-7,14-dioxa-4,10,19,20-tetraazatetracyclo[13.5.2.1^{2,6}.0^{18,21}]tricosa-1(20),2(23), 3,5,15(22),16,18(21)-heptaene;
10-methyl-7,14-dioxa-5,10,19,20-tetraazatetracyclo[13.5.2.1^{2,6}.0^{18,21}]tricosa-1(20),2(23), 3,5,15(22),16,18(21)-heptaen-9-one;
10-methyl-7-oxa-4,10,14,19,20-pentaazatetracyclo[13.5.2.1^{2,6}.0^{18,21}]tricosa-1(20),2,4, 6(23),15,17,21-heptaen-9-one;
10-methyl-9-oxo-7,14-dioxa-10,19,20-triazatetracyclo[13.5.2.1^{2,6}.0^{18,21}]tricosa-1(20),2,4,6(23),15,17,21-heptaene-4-carbonitrile;
7,13-dioxa-10,18,19-triazatetracyclo[12.5.2.1^{2,6}.0^{17,20}]docosa-1(19),2(22),3,5,14(21), 15,17(20)-heptaen-9-one;
7,14-dioxa-4,10,19,20,23-pentaazatetracyclo[13.5.2.1^{2,6}.0^{18,21}]tricosa-1(20),2(23),3,5, 15(22),16,18(21)-heptaen-9-one;
10-methyl-7,14-dioxa-3,10,19,20-tetraazatetracyclo[13.5.2.1^{2,6}.0^{18,21}]tricosa-1(20),2(23),3, 5,15(22),16,18(21)-heptaen-9-one;
4-fluoro-7,14-dioxa-10,19,20-triazatetracyclo[13.5.2.1^{2,6}.0^{18,21}]tricosa-1(20),2(23),3,5, 15(22),16,18(21)-heptaen-9-one;
7,14-dioxa-5,10,19,20,23-pentaazatetracyclo[13.5.2.1^{2,6}.0^{18,21}]tricosa-1(20),2(23),3,5, 15(22),16,18(21)-heptaen-9-one;
8,8,10-trimethyl-7,14-dioxa-4,10,19,20-tetraazatetracyclo[13.5.2.1^{2,6}.0^{18,21}]tricosa-1(20),2(23),3,5,15(22),16,18(21)-heptaen-9-one;
11-methyl-7,15-dioxa-4,11,20,21-tetraazatetracyclo[14.5.2.1^{2,6}.0^{19,22}]tricosa-1(21),2,4, 6(24),16,18,22-heptaen-10-one;
7,13-dioxa-4,10,18,19-tetraazatetracyclo[12.5.2.1^{2,6}.0^{17,20}]docosa-1(19),2(22),3,5,14(21), 15,17(20)-heptaen-9-one;
3-fluoro-10-methyl-7,14-dioxa-4,10,19,20-tetraazatetracyclo[13.5.2.1^{2,6}.0^{18,21}]tricosa-1(20),2,4,6(23),15(22),16,18(21)-hepten-9-one;
8,8-dimethyl-7,14-dioxa-4,10,19,20-tetraazatetracyclo[13.5.2.1^{2,6}.0^{18,21}]tricosa-1(20),2(23),3,5,15,17,21-heptaen-9-one;
12,12-difluoro-7,14-dioxa-4,10,19,20-tetraazatetracyclo[13.5.2.1^{2,6}.0^{18,21}]tricosa-1(20),2,4,6(23),15,17,21-heptaen-9-one;
4-bromo-7,14-dioxa-10,19,20-triazatetracyclo[13.5.2.1^{2,6}.0^{18,21}]tricosa-1(20),2(23),35,15,17,21-heptaen-9-one;
4-(piperidin-1-yl)-7,14-dioxa-10,19,20-triazatetracyclo[13.5.2.1^{2,6}.0^{18,21}]tricosa-1(20),2(23),3,5,15,17,21-heptaen-9-one;
4-(pyrrolidin-1-yl)-7,14-dioxa-10,19,20-triazatetracyclo[13.5.2.1^{2,6}.0^{18,21}]tricosa-1(20),2(23),3,5,15,17,21-heptaen-9-one;
4-(3,3-difluoropyrrolidin-1-yl)-7,14-dioxa-10,19,20-triazatetracyclo[13.5.2.1^{2,6}.0^{18,21}] tricosa-1(20),2(23),3,5,15,17,21-heptaen-9-one;
4-(morpholin-4-yl)-7,14-dioxa-10,19,20-triazatetracyclo[13.5.2.1^{2,6}.0^{18,21}]tricosa-1(20),2(23),3,5,13,17,21-heptaen-9-one;
4-(3,6-dihydro-2H-pyran-4-yl)-7,14-dioxa-10,19,20-triazatetracyclo[13.5.2.1^{2,6}.0^{18,21}] tricosa-1(20),2(23),3,5,15,17,21-heptaen-9-one;
4-[4-(cyclopropylmethyl)piperazin-1-yl]-7,14-dioxa-10,19,20-triazatetracyclo[13.5.2.1^{2,6}.0^{18,21}]tricosa-1(20),2(23),3,5,15,17,21-heptaen-9-one;
4-(5,6-dihydro-2H-pyran-3-yl)-7,14-dioxa-10,19,20-triazatetracyclo[13.5.2.1^{2,6}.0^{18,21}] tricosa-1(20),2(23),3,5,15,17,21-heptaen-9-one;
4-(2,5-dihydrofuran-3-yl)-7,14-dioxa-10,19,20-triazatetracyclo[13.5.2.1^{2,6}.0^{18,21}]tricosa-1(20),2(23),3,5,15,17,21-heptaen-9-one;
4-(piperazin-1-yl)-7,14-dioxa-10,19,20-triazatetracyclo[13.5.2.1^{2,6}.0^{18,21}]tricosa-1(20),2(23),3,5,15,17,21-heptaen-9-one;
4-(furan-3-yl)-7,14-dioxa-10,19,20-triazatetracyclo[13.5.2.1^{2,6}.0^{18,21}]tricosa-1(20),2(23),33,15,17,21-heptaen-9-one;
4-(oxolan-3-yl)-7,14-dioxa-10,19,20-triazatetracyclo[13.5.2.1^{2,6}.0^{18,21}]tricosa-1(20),2(23),3,5,15,17,21-heptaen-9-one;
(11R,12R)-11,12-dimethyl-7,14-dioxa-10,19,20-triazatetracyclo[13.5.2.1^{2,6}.0^{18,21}]tricosa-1(20),2,4,6(23),15(22),16,18(21)-heptaen-9-one;
4-[(3R,4R)-3,4-difluoropyrrolidin-1-yl]-7,14-dioxa-10,19,20-triazatetracyclo[13.5.2.1^{2,6}.0^{18,21}]tricosa-1(20),2(23),3,5,15,17,21-heptaen-9-one;
4-(4-cyclopropylpiperazin-1-yl)-7,14-dioxa-10,19,20-triazatetracyclo[13.5.2.1^{2,6}.0^{18,21}] tricosa-1(20),2(23),3,5,15,17,21-heptaen-9-one;
4-(oxan-2-yl)-7,14-dioxa-10,19,20-triazatetracyclo[13.5.2.1^{2,6}.0^{18,21}]tricosa-1(20),2(23),3,5,15,17,21-heptaen-9-one;
4-fluoro-14-oxa-10,19,20-triazatetracyclo[13.5.2.1^{2,6}.0^{18,21}]tricosa-1(20),2(23),3,5,15(22), 16,18(21)-heptaen-9-one;
14-oxa-4,10,19,20-tetraazatetracyclo[13.5.2.1^{2,6}.0^{18,21}]tricosa-1(20),2(23),3,5,15(22), 16,18(21)heptaen-9-one;
11-methyl-7,14-dioxa-11,19,20-triazatetracyclo[13.5.2.1^{2,6}.0^{18,21}]tricosa-(20),2(23),3,5, 15,17,21-heptaen-10-one;
8,14-dioxa-4,5,11,19,20,23-hexaazatetracyclo[13.5.2.1^{2,5}.0^{18,21}]tricosa-1(20),2(23),3, 15(22),16,18(21)-hexaen-10-one;
(12R)-12-methyl-13-oxa-4,5,9,18,19,22-hexaazatetracyclo[12.5.2.1^{2,5}. 0^{17,20}]docosa-1(19),2(22),3,14(21),15,17(20)-hexaen-8-one;
(12R)-12-methyl-13-oxa-4,5,9,18,19,22-hexaazatetracyclo[12.5.2.1^{2,5}.0^{17,20}]docosa-1(19),2(22),3,14(21),15,17(20)-hexaen-8-one;
(11S)-1-fluoro-13-oxa-4,5,9,18,19,22-hexaazatetracyclo[12.5.2.1^{2,5}.0^{17,20}]docosa-1(19),2(22),3,14(21),15,17(20)-hexaene-8-one;
(12S)-12-methyl-13-oxa-4,5,9,18,19,22-hexaazatetracyclo[12.5.2.1^{2,5}.0^{17,20}]docosa-1(19),2(22),3,14(21),15,17(20)-hexaen-8-one;
(11R)-11-fluoro-13-oxa-4,5,9,18,19,22-hexaazatetracyclo[12.5.2.1^{2,5}.0^{17,20}]docosa-1(19),2(22),3,14(21),15,17(20)-hexaen-8-one;
(11R,12S)-11,12-dimethyl-7,14-dioxa-10,19,20-triazatetracyclo[13.5.2.1^{2,6}.0^{18,21}]tricosa-1(20),2,4,6(23),15(22),16,18(21)-heptaene;
(8R or 8S,13S)-8,13-dimethyl-7,14-dioxa-4,10,19,20-tetraazatetracyclo[13.5.2.1^{2,6}.0^{18,21}] tricosa-1(20),2,4,6(23),15(22),16,18(21)-heptaene;
(8S or 8R,13S)-8,13-dimethyl-7,14-dioxa-4,10,19,20-tetraazatetracyclo[13.5.2.1^{2,6}.0^{18,21}] tricosa-1(20),2,4,6(23),15(22),16,18(21)-heptaene;
(11R,12R)-12-fluoro-11-methyl-7,14-dioxa-10,19,20-triazatetracyclo[13.5.2.1^{2,6}.0^{18,21}] tricosa-1(20),2,4,6(23),15(22),16,18(21)-heptaene;
(13S)-13-methyl-7,14-oxa-4,10,19,20-tetraazatetracyclo[13.5.2.1^{2,6}.0^{18,21}]tricosa-1(20),24,6(23),15(22),16,18(21)-heptaene;
(12S)-12-methyl-13-oxa-4,5,9,18,19-pentaazatetracyclo[12.5.2.1^{2,5}.0^{17,20}]docosa-1(19),2(22),3,14(21),15,17(20)-hexaene;
(7S or 7R,13R)-7,13-dimethyl-8,14-dioxa-4,11,19,20-tetraazatetracyclo[13.5.2.1^{2,6}.0^{18,21}] tricosa-1(20),2(23),3,5,15(22),16,18(21)-heptaene;
(7R or 7S,13R)-7,13-dimethyl-8,14-dioxa-4,11,19,20-tetraazatetracyclo[13.5.2.1^{2,6}.0^{18,21}] tricosa-1(20),2(23),3,5,15(22),16,18(21)-heptaene;
4-(morpholin-4-yl)-7,14-dioxa-11,19,20-triazatetracyclo[13.5.2.1^{2,6}.0^{18,21}]tricosa-1(20),2,4,6(23),15,17,21-heptaene;
7,14-dioxa-10,19,20,23-tetraazatetracyclo[13.5.2.1^{2,6}.0^{18,21}]tricosa-1(20),2,4,6(23),15, 17,21-heptaene;
7,14-dioxa-4,11,19,20-tetraazatetracyclo[13.5.2.1^{2,6}.0^{18,21}]tricosa-1(20),2,4,6(23),15, 17,21-heptaene;
10-methyl-4-(propan-2-yloxy)-7,14-dioxa-10,19,20-triazatetracyclo[13.5.2.1^{2,6}.0^{18,21}] tricosa-1(20),2,4,6(23),15,17,21-heptaene;
11-methyl-7,14-dioxa-10,19,20,23-tetraazatetracyclo[13.5.2.1^{2,6}.0^{18,21}]tricosa-1(20),2,4,6(23),15,17,21-heptaene;
11,11-difluoro-13-oxa-4,5,9,18,19,22-hexaazatetracyclo[12.5.2.1^{2,5}.0^{17,20}]docosa-1(19),2(22),3,14(21),15,17(20)-hexaen-8-one;
(6S,12R)-6,12-dimethyl-18-(oxan-2-yl)-13-oxa-4,5,9,18,19,22-hexaazatetracyclo[12.5.2.1^{2,5}.0^{17,20}]docosa-1(19),2(22),3,14(21),15,17(20)-hexaen-8-one;
(6S,12R)-6,9,12-trimethyl-18-(oxan-2-yl)-13-oxa-4,5,9,18,19,22-hexaazatetracyclo [12.5.2.1^{2,5}.0^{17,20}]docosa-1(19),2(22),3,14(21),15,17(20)-hexaen-8-one;
(6S,12R)-6,9,12-methyl-13-oxa-4,5,9,18,19,22-hexaazatetracyclo[12.5.2.1^{2,5}.0^{17,20}] docosa-1(19),2(22),3,14(21),15,17(20)-hexaene;
(12R)-12-methyl-9,13-dioxa-4,5,15,18,19-pentaazatetracyclo[12.5.2.1^{2,5}.0^{17,20}]docosa-1(19),2(22),3,14(21),15,17(20)-hexaene and
(12S)-12-methyl-8,11,14-trioxa-4,5,16,19,20-pentaazatetracyclo[13.5.2.1^{2,5}.0^{18,21}]tricosa-1(20),2(23),3,15(22),16,18(21)-hexaene.

143. A pharmaceutical composition comprising the compound according to claim 78, or an addition salt thereof with a pharmaceutically acceptable acid or base, in combination with one or more pharmaceutically acceptable excipients.

144. A method of treating a condition requiring an inhibitor of LRRK2 kinase activity in a subject in need thereof, comprising administration of the compound according to claim 78, alone or in combination with one or more pharmaceutically acceptable excipients.

145. The method according to claim 144, wherein the condition is selected from neurological diseases, endosomal-lysosomal disorders, inflammatory diseases, bacterial, viral and parasitic infections, cardiovascular diseases, autoimmune diseases and cancers.

146. The method according to claim 145, wherein the neurological disease is selected from Parkinson's disease, Alzheimer's disease, amyotrophic lateral sclerosis (ALS), dementia, diabetic neuropathy, age related memory disfunction, mild cognitive impairment, argyrophilic grain disease, Pick's disease, epilepsy, tauopathies, including progressive supranuclear palsy and corticobasal degeneration, synucleinopathies, including multiple system atrophy, frontotemporal dementia, inherited frontotemporal dementia and parkinsonism linked to chromosome 17 (FTDP-17), withdrawal symptoms/relapse associated with drug addiction, L-Dopa induced dyskinesia, ischemic stroke, traumatic brain injury, spinal cord injury and multiple sclerosis.

147. The method according to claim 146, wherein the neurological disease is selected from Parkinson's disease and Alzheimer's disease.

148. The method according to claim 145, wherein the endosomal-lysosomal disorder is selected from Niemann-Pick Type A, B or C disease, Gaucher's disease, Krabbe's disease, Fabry's disease and disorders with mitochondrial deficits.

149. The method according to claim 145, wherein the inflammatory disease is selected from vasculitis, pulmonary diseases, including chronic obstructive pulmonary disease, idiopathic pulmonary fibrosis, inflammatory myopathics, and ankylosing spondylitis.

150. The method according to claim 145, wherein the autoimmune disease is selected from Crohn's disease, inflammatory bowel disease, rheumatoid arthritis, ulcerative colitis, lupus, autoimmune hemolytic anemia, pure red cell aplasia, idiopathic thrombocytopenic purpura, type I diabetes mellitus, obesity, Evans syndrome, bullous skin disorders, Sjogren's syndrome, Devic's disease and leprosy.

151. The method according to claim 145, wherein cancer is selected from thyroid cancer, renal cancer, breast cancer, hormone-related cancer, adeno-and squamous lung cancer, non-small-cell lung cancer, colon cancer, prostate cancers, skin cancers, leukemias and lymphomas.

152. The method according to claim 145, wherein cardiovascular disease is stroke.

153. The method according to claim 145, wherein the bacterial or viral infections are selected from leprosy, tuberculosis, SARS-CoV, MERS-CoV and SARS-CoV-2, HIV, West Nile virus and chikungunya virus.
